(19) United States
(12) Patent Application Publication
(10) Pub. No.: US 2026/0109702 A1
(43) Pub. Date: Apr. 23, 2026

(54) NOVEL GSPT PROTEIN DEGRADER AND APPLICATION THEREOF

(71) Applicant: GENOSCO Inc., Billerica, MA (US)

(72) Inventors: Wongil LEE, Bedford, MA (US); Jang Sik CHOI, Billerica, MA (US); Ravi DASHNAMOORTHY, Shrewsbury, MA (US); Vinay SAGAR, Woburn, MA (US); Travis CLARK, Londonderry, NH (US); Miyong YONG, Belmont, MA (US); Anu GUPTA, Billerica, MA (US); Jaekyoo LEE, N. Andover, MA (US); Jong Sung KOH, Belmont, MA (US); William G. DEVINE, Woburn, MA (US)

(21) Appl. No.: 19/362,312

(22) Filed: Oct. 17, 2025

Related U.S. Application Data
(60) Provisional application No. 63/708,323, filed on Oct. 17, 2024.

(51) Int. Cl. C07D 487/10; A61K 31/454; A61K 31/4545; A61K 31/496; A61K 31/497; A61K 31/501; A61K 31/506; A61K 31/517; A61K 31/519; A61K 31/5377; A61K 31/5386; A61K 31/551; A61P 35/00; C07B 59/00; C07D 401/14; C07D 405/14; C07D 413/14; C07D 471/04; C07D 487/04; C07D 487/08; C07D 498/10

(52) U.S. Cl. CPC C07D 487/10; A61K 31/454; A61K 31/4545; A61K 31/496; A61K 31/497; A61K 31/501; A61K 31/506; A61K 31/517; A61K 31/519; A61K 31/5377; A61K 31/5386; A61K 31/551; A61P 35/00; C07B 59/002; C07D 401/14; C07D 405/14; C07D 413/14; C07D 471/04; C07D 487/04; C07D 487/08; C07D 498/10

(57) ABSTRACT

The present disclosure relates to novel compounds that act as modulators of cereblon (CRBN). The compounds promote CRBN-mediated ubiquitination and proteasomal degradation of oncogenic proteins such as GSPT1 and MYC, thereby suppressing abnormal cell growth. Pharmaceutical compositions containing the compounds and methods of using the compositions for the treatment of cancers, particularly solid tumors, are also provided.

### CROSS-REFERENCE TO RELATED APPLICATION

The present application claims priority to U.S. Patent Application No. 63/708,323 filed Oct. 17, 2024, which is incorporated herein by reference.

### TECHNICAL FIELD

The present disclosure relates to novel compounds as modulators of cereblon (CRBN). It also relates to methods of preparing the compounds, compositions comprising the compounds and methods of using the compounds and compositions to treat abnormal cell growth in mammals, particularly in humans.

### BACKGROUND OF THE INVENTION

Targeted protein degradation is an emerging therapeutic approach that utilizes the proteasome, an intracellular protein degradation machinery for the elimination of certain disease-causing proteins. At its core are small molecules known as molecular glues, which facilitate chemically induced proximity in the cell between cellular enzymes such as Cyclophilin A or E3 ligases and the protein of interest (disease-causing or progressing protein), leading to polyubiquitination of the target protein. Upon ubiquitination, proteins become susceptible to proteolysis through proteosome degradation. It is now possible to eliminate undruggable pathogenic proteins with high selectivity using molecular glues, which offers potential therapeutic opportunities for the treatment of many diseases, previously considered “undruggable diseases”.

Additionally, molecular glues, which bind to proteins and change their function, can enable these drugs to make these diseases more susceptible to conventional therapies. Particularly, immunomodulatory imide drugs (IMiD) including Thalidomide, Lenalidomide, Pomalidomide and their derivatives bind to cereblon, an adapter protein that recruits E3 ligase machinery, and function as molecular glue degrader. The FDA has approved these IMiDs for treatment of multiple myeloma, myelodysplastic syndrome, relapsed/refractory lymphomas, including follicular, marginal zone, and mantle cell lymphomas. These IMiDs, however, are less effective against most solid cancers, which constitute majority of all cancers. Therefore, it is crucial to design and develop functionalized IMiDs with protein degradation capabilities to effectively treat most of the solid cancers.

GSPT1 (G1 to S phase transition 1) is a protein involved in the release of newly synthesized proteins from the translational machinery. GSPT1/2 levels are higher in certain types of cancer and are associated with increased protein production and malignant activity, so it is considered a promising target for cancer therapy.

MYC, a well-known oncogene, functions as a master regulator of genes controlling cellular growth, metabolism, and proliferation. As transcription factors, members of the MYC protein family, including c-Myc, N-Myc, and L-Myc, promote the synthesis of messenger RNAs that encode proteins involved in these fundamental processes. Among the MYC family members, c-Myc is highly expressed in majority of cancers, while N-Myc and L-Myc are less frequently elevated. Dysregulated MYC signaling contributes to the pathogenesis of numerous malignancies, including lung squamous cell carcinoma, lung adenocarcinoma, liver cancer, skin cancer, colon cancer, bladder cancer, prostate cancer, breast cancer, gastric cancer, pancreatic cancer, ovarian cancer, uterine cancer, endometrial cancer, acute myeloid leukemia, and diffuse large B cell lymphoma. Despite its central role in oncogenesis, MYC remains a challenging therapeutic target due to its intrinsically disordered structure and lack of druggable binding pockets. As a result, it is widely regarded as an “undruggable” oncogene, and the development of indirect strategies to suppress MYC activity has become an important focus of modern cancer research.

Recent findings indicate that MYC-addicted tumors depend on GSPT1 to sustain elevated levels required for rapid tumor growth. Therefore, therapeutic agents capable of degrading GSPT1 can effectively disrupt MYC-driven oncogenic processes and induce tumor cell death. However, existing GSPT1 degraders have achieved only weak reductions in MYC expression, highlighting the need for more potent degraders capable of effectively suppressing MYC levels across diverse tumor types.

Additionally, aberrant activation of the WNT signaling pathway, driven by transcription factors such as TCF4, is known to induce MYC expression. Thus, GSPT1 degraders that can concurrently downregulate WNT signaling may offer an indirect yet powerful strategy for reducing MYC activity and improving therapeutic outcomes in MYC-dependent cancers.

Despite significant progress in the field of targeted protein degradation, existing molecular-glue degraders and IMiD-based therapeutics remain limited in their ability to address oncogenic drivers in solid tumors. Current GSPT1 degraders demonstrate only modest efficacy in reducing MYC levels, and none have yet achieved broad and durable antitumor activity across MYC-dependent malignancies. Accordingly, there remains an urgent and unmet need for the development of novel cereblon-mediated molecular glues capable of efficiently degrading GSPT1 and other oncogenic substrates such as MYC, thereby providing a new and effective therapeutic approach for the treatment of cancers including solid cancers.

This BACKGROUND section serves to provide context for the present disclosure, offering insight into the challenges addressed by the disclosed invention. The statements made herein are intended to facilitate a clearer understanding and are not to be construed as admissions regarding prior art.

### SUMMARY OF THE INVENTION

It is, therefore, an objective of the present disclosure to advance the state of the art of cereblon modulators and provide novel modulators for use in different diseases, including different types of cancers.

In an aspect, the present invention provides a compound of Formula (I), or a tautomer, stereoisomer or a mixture of stereoisomers, pharmaceutically acceptable salt, hydrate, or polymorph, co-crystal, or prodrug derivative thereof.

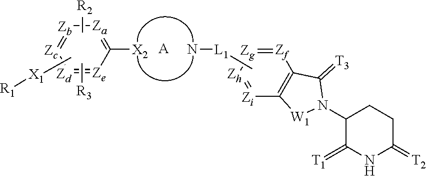

In another aspect, the present invention provides a pharmaceutical composition comprising the compound, salt, diastereomer, enantiomer, racemate, hydrate, solvate, prodrug of claim 1, or a combination thereof. In some embodiments, the composition may further comprise one or more additional therapeutic agents. In some embodiments, the additional therapeutic agent may be an anti-cancer agent.

In still another aspect, the present invention provides a method for treating, preventing, or ameliorating cancer, comprising administering to a subject a therapeutically effective amount of the pharmaceutical composition. In some embodiments, the cancer may be solid tumor or blood-borne cancer. In some embodiments, the cancer may be non-small cell lung cancer (e.g., squamous cell lung cancer), small cell lung cancer, hepatocellular carcinoma (HCC), breast cancer, and neuroendocrine cancer (e.g., neuroendocrine prostate cancer, such as castration-resistant neuroendocrine prostate cancer (NEPC) and lung neuroendocrine tumors (Lu-NETs)). In some embodiments, cancer may be leukemias (e.g. acute myelogenous leukemia (AML)) and myelomas (e.g. multiple myeloma (MM)). In some embodiments, the composition may degrade a target protein via cereblon, leading to direct degradation of GSPT levels, while indirectly reducing MYC protein levels in certain tumors.

### BRIEF DESCRIPTION OF THE DRAWINGS

FIG. 1A is a proteomic profiling analysis of LK2 (lung squamous cell cancer) cells treated with 100 nM compound 1-11 for 6 hours, showing that GSPT1 and GSPT2 are the most significantly reduced (degraded) proteins, each exhibiting with >1 Log 2 fold-change with P<0.001 and FDR<0.05 among more than 7,000 proteins.

FIG. 1B shows proteomic profiling of LK2 (lung squamous cell cancer) cells treated with 100 nM compound 2-4 for 6 hours, demonstrating that GSPT1 is the most significantly reduced (degraded) protein with >1 Log 2 fold-change, P<0.001, and FDR<0.05 among more than 7,000 proteins.

FIG. 1C shows proteomic profiling of H1155 (lung squamous cell cancer) cells treated with 100 nM compound 2-4 for 2 hours, showing that GSPT1 and GSPT2 are the most significantly reduced (degraded) protein with >1 Log 2 fold-change, P<0.001, and FDR<0.05 among more than 7,000 proteins.

FIG. 1D is a proteomic profiling analysis of HUH7 (hepatocellular cancer) cells treated with 100 nM compound 2-4 for 6 hours, showing that GSPT1 and GSPT2 are the topmost significantly reduced (degraded) protein with >1 Log 2 fold-change, P<0.001, and FDR<0.05 among more than 7,000 proteins.

FIG. 2A shows Western blot analysis of LK2 (lung squamous cell cancer) cells treated with compound 2-4 (0-300 nM) and harvested at 2, 8 or 18 hours, showing that deep GSPT1 and GSPT2 degradation commences between 2 and 8 hours at compound concentrations>10 nM, while more pronounced degradation is observed at 18 hours even at 1 nM. A reduction in c-Myc levels is observed at 8 hours at >100 nM with compound 2-4 and becomes more evident at 18 hours at ≥30 nM.

FIGS. 2B-2E show Western blot analysis of LK2 and H1155 (lung squamous cell cancer), HUH7 (hepatocellular cancer) and COLO201 (colon adenocarcinoma) cells treated with compounds 1-11 or 2-4 (0, 10, 30 & 100 nM) and harvested at 18 hours. The figures show that deep GSPT1 or GSPT2 degradation occurs in all tested cell lines beginning at 10 nM. In c-MYC-driven LK2 cells, compound 1-11 exhibits a stronger reduction in c-Myc protein levels compared to compound 2-4. In N-MYC-driven cancer cell lines (H1155, HUH7, and COLO201), both compounds 1-11 and 2-4 produce significant N-Myc reduction, with compound 1-11 showing greater potency at 10 nM. Neither compound reduces CRBN protein levels, and β-actin signals confirm equal protein loading.

FIG. 3A shows the results from LK2 (lung squamous cell cancer) cells stably transfected with a constitutively active c-MYC reporter-Luciferase construct, demonstrating that treatment with compound 1-11 for 16 hours results in 50% inhibition of reporter activity at 10 nM.

FIG. 3B shows that the results from LK2 (lung squamous cell cancer) cells stably transfected with a constitutively active c-MYC reporter-Luciferase construct, demonstrating that treatment with compound 2-4 for 16 hours results in 50% inhibition of reporter activity at >30 nM.

FIG. 3C demonstrates that, in LK2 (lung squamous cell cancer) cells stably transfected with a constitutively active TCF4/LEF reporter-Luciferase construct, treatment with compound 2-4 for 16 hours results in 50% inhibition of reporter activity at >10 nM.

FIG. 3D demonstrates that, in LK2 (lung squamous cell cancer), Hep3B (hepatacellular carcinoma) or SW948 (colorectal adenocarcinoma) cells stably transfected with a constitutively active TCF4/LEF reporter-Luciferase construct, treatment with compound 2-4 for 6 hours results in downregulation of reporter activity with 50% inhibition of reporter activity at >30 nM observed in Hep3B and SW948.

FIGS. 4A-4E illustrate the in vivo efficacy of the compounds of certain embodiments of the invention in tumor xenograft models.

### DETAILED DESCRIPTION OF THE INVENTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

### 1. Compounds

In one aspect, the present invention provides a compound represented by Formula (I), a pharmaceutically acceptable salt, diastereomer, enantiomer, racemate, hydrate, solvate, or prodrug thereof:

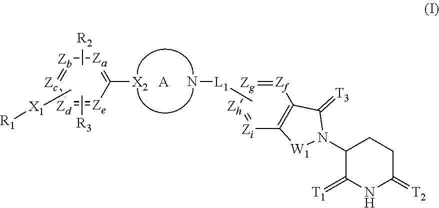

wherein:
L₁ is

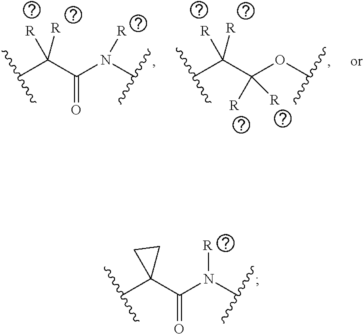

X₁ is a covalent bond, —O—, —S—, CHR₆ₐ, CR₆ₐR_{6b}, or NR₆ₐ;
X₂ is N, C, or CH;
Zₐ, Z_{b}, Z_{c}, Z_{d}, and Zₑ each are independently N or CR₅;
Z_{f}, Z_{g}, Zₕ, and Zᵢ each are independently N or CR₅;
T₁, T₂, and T₃ each are independently oxygen or sulfur;
W₁ is CH₂ or C(O);

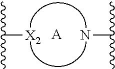

is 4-12 membered heterocyclic, 5-13 membered fused heterocycle, 5-13 membered bridged heterocycle, or 5-13 membered spiro heterocycle wherein 4-12 membered heterocyclic, 5-13 membered fused heterocycle, 5-13 membered bridged heterocycle, or 5-13 membered spiro heterocycle, each of which is optionally substituted with one or more groups independently selected from the group consisting of —C₁₋₃ alkyl, —C₁₋₃ haloalkyl, CD₃, -oxo, —C₀₋₃ NR₆ₐR_{6b}, halogen, and —C₀₋₃ OH;
R₁ is hydrogen, 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, or 3-11 membered heterocycle, wherein 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, or 3-11 membered heterocycle, which is optionally substituted with one or more groups independently selected from the group consisting of halogen, —CN, CD₃, —NO₂, —C(O)R₆ₐ, —NR₆ₐR_{6b}, —S(O)₂NR₆ₐR_{6b}, —NR₆ₐS(O)₂R_{6b}, C₁₋₆ haloalkoxy, —COOH, —CHO, furan, benzyloxy, —NR₆ₐC(O)R_{6b}, NR₆ₐC(O)NHR_{6b}, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, and C₁₋₁₅ heteroalkyl, wherein C₁₋₆ haloalkoxy, —COOH, —CHO, furan, benzyloxy, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, or C₁₋₁₅ heteroalkyl is optionally substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkyNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙO(CH₂)ₘ—NR₆ₐR_{6b}, —((CH₂)ₙO)ₘ—(CH₂)ₒ—NR₆ₐR_{6b}, —NR₆ₐC(O)(CH₂)ₙOH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR_{6b}, and —NR₆ₐ(CH₂)ₙNR_{6b}R_{6c};
R₂ and R₃ each are independently hydrogen, halogen, —CD₃, —CN, —NO₂, —S(O)₂NR₆ₐR_{6b}, —NR₆ₐS(O)₂R_{6b}, —C₁₋₆ haloalkoxy, —COOH, —CHO, —C(O)R₆ₐ, —NR₆ₐC(O)R_{6b}, —(CO)NR₆ₐR_{6b}, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl, wherein —C₁₋₆ haloalkoxy, —CHO, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl is optionally substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙ—O—(CH₂)ₘ—NR₆ₐR_{6b}, —((CH₂)ₙO)ₘ—(CH₂)ₒ—NR₆ₐR_{6b}, —NR₆ₐC(O)(CH₂)ₙCH₂OH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR₆ₐ, and —NR₆ₐ(CH₂)ₙNR_{6b}R_{6c};
R₄ₐ, R_{4b}, R₅ₐ, and R_{5b} each are independently hydrogen, halogen, —OH, —OCH₃, —CH₂CH₃, —CH₃, —CD₃, CN, —CF₃, —CHF₂, —CMeF₂, —CH₂CF₃, —CH₂CHF₂, each of which is optionally substituted with one or more groups independently selected from the group consisting of C₃₋₅ cycloalkyl, C₁₋₆ alkyl, C₁₋₆ alkylOH, C₁₋₆ alkylNH₂, and C₁₋₆ alkylNHR₆ₐ;
R₅ is hydrogen, halogen, —OH, —NH₂, —NHCH₃, C₁₋₃ alkyl, or C₁₋₃ alkoxy;
R₆ₐ, R_{6b}, and R_{6c} each are independently hydrogen, —CD₃, —S(O)₂R₇ₐ, aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl, wherein aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl is optionally substituted with one or more groups independently selected from the group consisting of —C₀₋₆ alkylNR₇ₐR_{7b}, —NR₇ₐC(O)(CH₂)ₙOH, —NR₇ₐC(O)(CH₂)ₙ—O—(CH₂)ₘ—OH, —NR₇ₐC(O)C₁₋₆ alkylNR_{7b}R_{7c}, —NR₇ₐC(O)C₁₋₆ alkylOR_{7b}, —NR₇ₐC(O)C₁₋₆ alkylSR_{7b}, —C(O)R₇ₐ, —C(O)C₀₋₆ alkylOR₇ₐ, —C(O)C₀₋₆ alkylNR₇ₐR_{7b}, —C(O)NR₇ₐR_{7b}, —C(O)NR₇ₐC₁₋₆ alkylNR_{7b}R_{7c}, —C(O)OC₁₋₆ alkylNR₇ₐR_{7b}, —C(O)OC₁₋₆ alkylOR₇ₐ, —(O(CH₂)ₙ)ₘOR₇a, —O(CH₂)ₙC₁₋₆ cycloalkylNR₇ₐR_{7b}, —(CH₂)ₙO(CH₂)ₘ—NR₇ₐR_{7b}, —((CH₂)ₙO)ₘ(CH₂)ₒ—NR₇ₐR₇ₙ, —NR₇ₐC(O)(CH₂)ₙOH, —(O(CH₂)ₙ)ₘSR₇ₐ, —(O(CH₂)ₙ)ₘNR₇ₐR_{7b}, —(NR₇ₐ(CH₂)ₙ)ₘOR_{7b}, —(NR₇ₐ(CH₂)ₙ)ₘSR_{7b}, and —NR₇ₐ(CH₂)ₙNR_{7b}R_{7c};
R₇ₐ, R_{7b}, and R_{7c} each are independently hydrogen, halogen, —C₀₋₃ CN, —C₀₋₃ C(O)NH₂, —C₀₋₃ C(O)NHCH₃, —C₀₋₃ C(O)N(CH₃)₂, —CD₃, benzyl, —C₃₋₆ cycloalkyl, 4-7 membered heterocyclic, C₁₋₆ alkyl, —C₀₋₆ alkylOH, —C₀₋₆ alkylOCH3, —C₀₋₆ alkylNH₂, —C₀₋₆ alkylNHCH3, —C₀₋₆ haloalkylNHCH₃, —C₀₋₆ alkylC(O)OH, —C₀₋₆ alkylC(O)OCH₃, —C₀₋₆ alkylSH, —C₁₋₆ alkoxy, —C₂₋₆ alkenyl, —C₂₋₆ alkynyl, —C₁₋₆ haloalkyl, —C₁₋₁₀ heteroalkyl, —C(O)C₁₋₆ OH, —C(O)C₁₋₆ NH₂, or —C(O)C₁₋₃ NHC₁₋₃ alkyl; and
n, m, and o each are independently an integer of 1, 2, 3, 4, or 5.

In some embodiments, Ring A may be selected from the group consisting of azetidine, pyrrolidine, piperidine, 1,2,3,6-tetrahydropyridine, piperazine, 3,6-diazabicyclo[3.1.1]heptane, piperazin-2-one, 1,4-diazepane, piperidine-2-one, 3-azaspiro[5.5]undecane, 3,9-diazaspiro[5.5]undecane, 2,6-diazaspiro[3.4]octane, and 2,6-diazaspiro[3.3]heptane. Preferably, it may be optionally substituted with one or two substituents independently selected from the group consisting of cyclopropyl, -Me, Oxo, and —CF₃.

In some embodiments, R₁ may be selected from the group consisting of hydrogen, 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, and 3-11 membered heterocycle. In some modified embodiments, it may be substituted with one or more groups independently selected from the group consisting of halogen, —CN, CD₃, —NO₂, —C(O)R₆ₐ, —NR₆ₐR_{6b}, —S(O)₂NR₆ₐR_{6b}, —NR₆ₐS(O)₂R_{6b}, C₁₋₆ haloalkoxy, —COOH, —CHO, furan, benzyloxy, —NR₆ₐC(O)R_{6b}, NR₆ₐC(O)NHR_{6b}, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, or C₁₋₁₅ heteroalkyl, wherein C₁₋₆ haloalkoxy, —COOH, —CHO, furan, benzyloxy, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, and C₁₋₁₅ heteroalkyl. In some other modified embodiments, C₁₋₆ haloalkoxy, —COOH, —CHO, furan, benzyloxy, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, or C₁₋₁₅ heteroalkyl may be substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylN_{Ra}R_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙO(CH₂)ₘ—NR₆ₐR_{6b}, —((CH₂)ₙO)ₘ—(CH₂)ₒ—NR₆ₐR_{6b}, —NR₆ₐC(O)(CH₂)ₙOH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR_{6b}, and —NR₆ₐ(CH₂)ₙNR_{6b}R_{6c}.

In some embodiments, R₂ and R₃ may be independently selected from the group consisting of hydrogen, halogen, —CD₃, —CN, —NO₂, —S(O)₂NR₆ₐR_{6b}, —NR₆ₐS(O)₂R_{6b}, —C₁₋₆ haloalkoxy, —COOH, —CHO, —C(O)R₆ₐ, —NR₆ₐC(O)R_{6b}, —(CO)NR₆ₐR_{6b}, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, and C₁₋₁₀ heteroalkyl. In some modified embodiments, C₁₋₆ haloalkoxy, —CHO, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl may be substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙ—O—(CH₂)ₘ—NR₆ₐR_{6b}, —((CH₂)ₙO)ₘ—(CH₂)ₒ—NR₆ₐR_{6b}, —NR₆ₐC(O)(CH₂)ₙCH₂OH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR₆ₐ, and —NR₆ₐ(CH₂)ₙNR_{6b}R_{6c}.

In some embodiments, R₄ₐ, R_{4b}, R₅ₐ, and R_{5b} in L may be independently selected from the group consisting of hydrogen, halogen, —OH, —OCH₃, —CH₂CH₃, —CH₃, —CD₃, CN, —CF₃, —CHF₂, —CMeF₂, —CH₂CF₃, and —CH₂CHF₂. They each may be optionally substituted with one or more groups independently selected from the group consisting of C₃₋₅ cycloalkyl, C₁₋₆ alkyl, C₁₋₆ alkylOH, C₁₋₆ alkylNH₂, and C₁₋₆ alkylNHR₆ₐ.

In some embodiments, R₅ may be selected from the group consisting of hydrogen, halogen, —OH, —NH₂, —NHCH₃, C₁₋₃ alkyl, and C₁₋₃ alkoxy.

In some embodiments, R₆ₐ, R_{6b}, and R_{6c} may be independently selected from the group consisting of hydrogen, —CD₃, —S(O)₂R₇ₐ, aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, and C₁₋₁₅ heteroalkyl. In some modified embodiments, they each may be optionally substituted with one or more groups independently selected from the group consisting of —NR₇ₐR_{7b}, —NR₇ₐC(O)(CH₂)ₙOH, —NR₇ₐC(O)(CH₂)ₙ—O—(CH₂)ₙ—OH, —NR₇ₐC(O)—C₁₋₆ alkylNH₂, -alkylNHR₇ₐ, —NR₇ₐC(O)—C₁₋₆ alkylOH, —NR₇ₐC(O)—C₁₋₆-alkylSH, —C(O)O—C₁₋₆ alkylNH₂, -alkylNHR₇ₐ—C(O)O—C₁₋₆-alkylOH, —(CH₂)ₙ—O—(CH₂)ₙ—NR₇ₐR_{7b}, —NR₇ₐC(O)(CH₂)ₙCH₂R_{7b}OH, —C(O)NR₇ₐ(CH)ₙNR₇ₐR_{7b}, —(O(CH₂)ₙ)ₘOH, —(O(CH₂)ₙ)ₘSH, —(O(CH₂)ₙ)ₘNR₇ₐR_{7b}, —(NR₇ₐ(CH₂)ₙ)ₘOH, —(NR₇ₐ(CH₂)ₙ)ₘSH, and —(NR₇ₐ(CH₂)ₙ)ₘNR₇ₐR_{7b}. In some other modified embodiments, aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl may optionally be substituted with one or more groups independently selected from the group consisting of —C₀₋₆ alkylNR₇ₐR_{7b}, —NR₇ₐC(O)(CH₂)ₙOH, —NR₇ₐC(O)(CH₂)ₙ—O—(CH₂)ₘ—OH, —NR₇ₐC(O)C₁₋₆ alkylNR_{7b}R_{7c}, —NR₇ₐC(O)C₁₋₆ alkylOR_{7b}, —NR₇ₐC(O)C₁₋₆ alkylSR_{7b}, —C(O)R₇ₐ, —C(O)C₀₋₆ alkylOR₇ₐ, —C(O)C₀₋₆ alkylNR₇ₐR_{7b}, —C(O)NR₇ₐR_{7b}, —C(O)NR₇ₐC₁₋₆ alkylNR_{7b}R_{7c}, —C(O)OC₁₋₆ alkylNR₇ₐR_{7b}, —C(O)OC₁₋₆ alkylOR₇ₐ, —(O(CH₂)ₙ)ₘOR₇a, —O(CH₂)ₙC₁₋₆ cycloalkylNR₇ₐR_{7b}, —(CH₂)ₙO(CH₂)ₘ—NR₇ₐR_{7b}, —((CH₂)ₙO)ₘ(CH₂)ₒ—NR₇ₐR_{7b}, —NR₇ₐC(O)(CH₂)ₙOH, —(O(CH₂)ₙ)ₘSR₇ₐ, —(O(CH₂)ₙ)ₘNR₇ₐR_{7b}, —(NR₇ₐ(CH₂)ₙ)ₘOR_{7b}, —(NR₇ₐ(CH₂)ₙ)ₘSR_{7b}, and —NR₇ₐ(CH₂)ₙNR_{7b}R_{7c}.

In some embodiments, R₇ₐ, R_{7b}, and R_{7c} may be independently selected from the group consisting of hydrogen, halogen, —C₀₋₃ CN, —C₀₋₃ C(O)NH₂, —C₀₋₃ C(O)NHCH₃, —C₀₋₃ C(O)N(CH₃)₂, —CD₃, benzyl, —C₃₋₆ cycloalkyl, 4-7 membered heterocyclic, C₁₋₆ alkyl, —C₀₋₆ alkylOH, —C₀₋₆ alkylOCH3, —C₀₋₆ alkylNH₂, —C₀₋₆ alkylNHCH3, —C₀₋₆ haloalkylNHCH₃, —C₀₋₆ alkylC(O)OH, —C₀₋₆ alkylC(O)OCH₃, —C₀₋₆ alkylSH, —C₁₋₆ alkoxy, —C₂₋₆ alkenyl, —C₂₋₆ alkynyl, —C₁₋₆ haloalkyl, —C₁₋₁₀ heteroalkyl, —C(O)C₁₋₆ OH, —C(O)C₁₋₆ NH₂, and —C(O)C₁₋₃ NHC₁₋₃ alkyl.

In some embodiments, n, m, and o may be independently selected from integers of 1, 2, and 3.

Non-limiting examples of the compounds of the present invention may include:
N-{2-[(S)-2,6-dioxo-3-piperidyl]-1-oxo-5-isoindolinyl}[3-(p-bromophenyl)-1-azetidinyl]acetamide
N-{2-[(S)-2,6-dioxo-3-piperidyl]-1-oxo-5-isoindolinyl}[4-(o-fluorophenyl)-1-piperazinyl]acetamide
N-{2-[(S)-2,6-dioxo-3-piperidyl]-1-oxo-5-isoindolinyl}[4-(o-methoxyphenyl)-1-piperazinyl]acetamide
N-{2-[(S)-2,6-dioxo-3-piperidyl]-1-oxo-5-isoindolinyl}{4-[2-methoxy-4-(2-pyridylamino)phenyl]-1-piperazinyl}acetamide
N-{2-[(S)-2,6-dioxo-3-piperidyl]-1-oxo-5-isoindolinyl}{4-[4-(2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(2-pyridylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}propionamide
N-{2-[(S)-2,6-dioxo-3-piperidyl]-1-oxo-5-isoindolinyl}2-{4-[2-methoxy-4-(2-pyridylamino)phenyl]-1-piperazinyl}propionamide
N-{2-[(S)-2,6-dioxo-3-piperidyl]-1-oxo-5-isoindolinyl}2-{4-[4-(2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[4-(6-chloro-4-pyrimidinylamino)-2-fluorophenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(6-chloro-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(2-pyrimidinylamino)phenyl]-1-piperazinyl}acetamide
N-{2-[(S)-2,6-dioxo-3-piperidyl]-1-oxo-5-isoindolinyl}{4-[4-amino-2-(trifluoromethyl)phenyl]-1-piperazinyl}acetamide
N-{2-[(S)-2,6-dioxo-3-piperidyl]-1-oxo-5-isoindolinyl}{4-[4-(4-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[p-(2-pyridylamino)phenyl]-1-azetidinyl}acetamide
N-{2-[(S)-2,6-dioxo-3-piperidyl]-1-oxo-5-isoindolinyl}2-{3-[p-(2-pyridylamino)phenyl]-1-azetidinyl}propionamide
N-{2-[(S)-2,6-dioxo-3-piperidyl]-1-oxo-5-isoindolinyl}{4-[2-methoxy-4-(2-pyrimidinylamino)phenyl]-1-piperazinyl}acetamide
N-{2-[(S)-2,6-dioxo-3-piperidyl]-1-oxo-5-isoindolinyl}{4-[2-chloro-4-(4-pyrimidinylamino)phenyl]-1-piperazinyl}acetamide
N-{2-[(S)-2,6-dioxo-3-piperidyl]-1-oxo-5-isoindolinyl}{4-[3-methoxy-4-(2-pyrimidinylamino)phenyl]-1-piperazinyl}acetamide
N-{2-[(S)-2,6-dioxo-3-piperidyl]-1-oxo-5-isoindolinyl}{4-[3-methoxy-4-(2-pyridylamino)phenyl]-1-piperazinyl}acetamide
N-{2-[(S)-2,6-dioxo-3-piperidyl]-1-oxo-5-isoindolinyl}{4-[2-fluoro-4-(2-pyridylamino)phenyl]-1-piperazinyl}acetamide
N-{2-[(S)-2,6-dioxo-3-piperidyl]-1-oxo-5-isoindolinyl}{4-[2-fluoro-4-(2-pyrimidinylamino)phenyl]-1-piperazinyl}acetamide
N-{2-[(S)-2,6-dioxo-3-piperidyl]-1-oxo-5-isoindolinyl}{4-[2-fluoro-4-(4-pyrimidinylamino)phenyl]-1-piperazinyl}acetamide
N-{2-[(S)-2,6-dioxo-3-piperidyl]-1-oxo-5-isoindolinyl}{4-[p-(2-pyridylamino)phenyl]-1-piperazinyl}acetamide
N-{2-[(S)-2,6-dioxo-3-piperidyl]-1-oxo-5-isoindolinyl}{4-[p-(2-pyrimidinylamino)phenyl]-1-piperazinyl}acetamide
N-{2-[(S)-2,6-dioxo-3-piperidyl]-1-oxo-5-isoindolinyl}{4-[4-(2-pyridylamino)-2-tolyl]-1-piperazinyl}acetamide
N-{2-[(S)-2,6-dioxo-3-piperidyl]-1-oxo-5-isoindolinyl}{4-[4-(2-pyrimidinylamino)-2-tolyl]-1-piperazinyl}acetamide
N-{2-[(S)-2,6-dioxo-3-piperidyl]-1-oxo-5-isoindolinyl}{4-[4-(6-chloro-4-pyrimidinylamino)-2-tolyl]-1-piperazinyl}acetamide
N-{2-[(S)-2,6-dioxo-3-piperidyl]-1-oxo-5-isoindolinyl}(S)-2-{4-[2-chloro-4-(2-pyridylamino)phenyl]-1-piperazinyl}propionamide
N-{2-[(S)-2,6-dioxo-3-piperidyl]-1-oxo-5-isoindolinyl}(R)-2-{4-[2-chloro-4-(2-pyridylamino)phenyl]-1-piperazinyl}propionamide
N-{2-[(S)-2,6-dioxo-3-piperidyl]-1-oxo-5-isoindolinyl}{4-[4-(4,6-dimethyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}2-{4-[2-chloro-4-(2-pyrimidinylamino)phenyl]-1-piperazinyl}propionamide
N-{2-[(S)-2,6-dioxo-3-piperidyl]-1-oxo-5-isoindolinyl}2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}2-{4-[4-(6-chloro-4-pyrimidinylamino)-2-tolyl]-1-piperazinyl}propionamide
N-{2-[(S)-2,6-dioxo-3-piperidyl]-1-oxo-5-isoindolinyl}{4-[2,6-difluoro-4-(2-pyrimidinylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}butyramide
N-{2-[(S)-2,6-dioxo-3-piperidyl]-1-oxo-5-isoindolinyl}{4-[2-chloro-4-(4,6-dimethyl-2-pyrimidinylamino)phenyl]-1-piperazinyl}acetamide
N-{2-[(S)-2,6-dioxo-3-piperidyl]-1-oxo-5-isoindolinyl}{4-[4-(4,6-dimethyl-2-pyrimidinylamino)-2-methoxyphenyl]-1-piperazinyl}acetamide
N-{2-[(S)-2,6-dioxo-3-piperidyl]-1-oxo-5-isoindolinyl}(R)-2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{4-[2-cyano-4-(4,6-dimethyl-2-pyrimidinylamino)phenyl]-1-piperazinyl}acetamide
N-{2-[(S)-2,6-dioxo-3-piperidyl]-1-oxo-5-isoindolinyl}{4-[4-(4-cyano-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(6-methyl-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}(R)-2-{4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{3-oxo-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{(R)-2-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-cyano-4-(2-pyrimidinylamino)phenyl]-1-piperazinyl}acetamide
N-{2-[(S)-2,6-dioxo-3-piperidyl]-1-oxo-5-isoindolinyl}(S)-2-{4-[2-cyano-4-(2-pyrimidinylamino)phenyl]-1-piperazinyl}propionamide
N-{2-[(S)-2,6-dioxo-3-piperidyl]-1-oxo-5-isoindolinyl}{(S)-2-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{(R)-3-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methoxy-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[4-(4-methoxy-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{(S)-3-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}[4-(4-amino-2-chlorophenyl)-1-piperazinyl]acetamide
N-{2-[(S)-2,6-dioxo-3-piperidyl]-1-oxo-5-isoindolinyl}{4-[4-(4,6-dimethyl-2-pyrimidinylamino)-2-tolyl]-1-piperazinyl}acetamide
N-{2-[(S)-2,6-dioxo-3-piperidyl]-1-oxo-5-isoindolinyl}{4-[4-(2,6-dimethyl-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[2-methoxy-4-(2-pyrimidinylamino)phenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyrimidinylamino)-2-methoxyphenyl]-1-piperazinyl}propionamide
N-{2-[(S)-2,6-dioxo-3-piperidyl]-1-oxo-5-isoindolinyl}{4-[4-(5-methyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methoxy-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1,4-diazepan-1-yl}acetamide
N-{2-[(S)-2,6-dioxo-3-piperidyl]-1-oxo-5-isoindolinyl}{4-[4-(methylamino)-2-(trifluoromethyl)phenyl]-1-piperazinyl}acetamide
N-{2-[(S)-2,6-dioxo-3-piperidyl]-1-oxo-5-isoindolinyl}{4-[4-(dimethylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(5-methyl-2-pyrimidinylamino)phenyl]-1-piperazinyl}acetamide
N-{2-[(S)-2,6-dioxo-3-piperidyl]-1-oxo-5-isoindolinyl}{4-[2-chloro-4-(methylamino)phenyl]-1-piperazinyl}acetamide
N-{2-[(S)-2,6-dioxo-3-piperidyl]-1-oxo-5-isoindolinyl}{4-[2-chloro-4-(dimethylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}-2-methylpropionamide
N-{2-[(S)-2,6-dioxo-3-piperidyl]-1-oxo-5-isoindolinyl}{3-[2-chloro-4-(2-pyridylamino)phenyl]-1-azetidinyl}acetamide
N-{2-[(S)-2,6-dioxo-3-piperidyl]-1-oxo-5-isoindolinyl}{3-[2-chloro-4-(4-methyl-2-pyridylamino)phenyl]-1-azetidinyl}acetamide
N-{2-[(S)-2,6-dioxo-3-piperidyl]-1-oxo-5-isoindolinyl}(S)-2-{4-[2-chloro-4-(4,6-dimethyl-2-pyrimidinylamino)phenyl]-1-piperazinyl}propionamide
N-{2-[(S)-2,6-dioxo-3-piperidyl]-1-oxo-5-isoindolinyl}{4-[2-chloro-4-(4-methyl-2-pyrimidinylamino)phenyl]-1-piperazinyl}acetamide
N-{2-[(S)-2,6-dioxo-3-piperidyl]-1-oxo-5-isoindolinyl}{4-[2-chloro-4-(2,6-dimethyl-4-pyrimidinylamino)phenyl]-1-piperazinyl}acetamide
N-{2-[(S)-2,6-dioxo-3-piperidyl]-1-oxo-5-isoindolinyl}{3-[4-(4-methyl-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[2-chloro-4-(2-pyrimidinylamino)phenyl]-1-azetidinyl}acetamide
N-{2-[(S)-2,6-dioxo-3-piperidyl]-1-oxo-5-isoindolinyl}{4-[2-chloro-4-(4-cyano-2-pyridylamino)phenyl]-1-piperazinyl}acetamide
N-{2-[(S)-2,6-dioxo-3-piperidyl]-1-oxo-5-isoindolinyl}{3-[4-(4,6-dimethyl-2-pyrimidinylamino)-2-(trifluoromethyl)phenyl]-3,6-diazabicyclo[3.1.1]hept-6-yl}acetamide
N-{2-[(S)-2,6-dioxo-3-piperidyl]-1-oxo-5-isoindolinyl}{(R)-3-methyl-4-[4-nitro-2-(trifluoromethyl)phenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-4,7-diaza-7-spiro[2.5]octyl}acetamide
N-{2-[(S)-2,6-dioxo-3-piperidyl]-1-oxo-5-isoindolinyl}{4-[4-(4-methyl-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(4-methyl-2-pyridylamino)phenyl]-1-piperazinyl}acetamide
N-{2-[(S)-2,6-dioxo-3-piperidyl]-1-oxo-5-isoindolinyl}{3-[4-(2-pyrimidinylamino)-2-(trifluoromethyl)phenyl]-3,6-diazabicyclo[3.1.1]hept-6-yl}acetamide
N-{2-[(S)-2,6-dioxo-3-piperidyl]-1-oxo-5-isoindolinyl}{(S)-4-[4-amino-2-(trifluoromethyl)phenyl]-3-methyl-1-piperazinyl}acetamide
N-{2-[(S)-2,6-dioxo-3-piperidyl]-1-oxo-5-isoindolinyl}{(R)-4-[4-(4,6-dimethyl-2-pyrimidinylamino)-2-(trifluoromethyl)phenyl]-3-methyl-1-piperazinyl}acetamide
N-{2-[(S)-2,6-dioxo-3-piperidyl]-1-oxo-5-isoindolinyl}{(R)-4-[4-(4-cyano-2-pyridylamino)-2-(trifluoromethyl)phenyl]-3-methyl-1-piperazinyl}acetamide
N-{2-[(S)-2,6-dioxo-3-piperidyl]-1-oxo-5-isoindolinyl}(R)-2-{4-[2-chloro-4-(4,6-dimethyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-cyano-2-pyridylamino)phenyl]-1-piperazinyl}propionamide
N-{2-[(S)-2,6-dioxo-3-piperidyl]-1-oxo-5-isoindolinyl}(R)-2-{4-[4-(2,6-dimethyl-4-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(5-methyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(4-methyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(4-methyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(R)-2-{3-[4-(2-pyrimidinylamino)-2-(trifluoromethyl)phenyl]-1-azetidinyl}propionamide
N-{2-[(S)-2,6-dioxo-3-piperidyl]-1-oxo-5-isoindolinyl}(R)-2-{3-[2-chloro-4-(2-pyrimidinylamino)phenyl]-1-azetidinyl}propionamide
N-{2-[(S)-2,6-dioxo-3-piperidyl]-1-oxo-5-isoindolinyl}{4-[4-(N-methyl-N-2-pyrimidinylamino)-2-chlorophenyl]-1-piperazinyl}acetamide
N-{2-[(S)-2,6-dioxo-3-piperidyl]-1-oxo-5-isoindolinyl}[4-(4-bromo-2-chlorophenyl)-1-piperazinyl]acetamide
N-{2-[(S)-2,6-dioxo-3-piperidyl]-1-oxo-5-isoindolinyl}(R)-2-{(R)-4-[4-(4,6-dimethyl-2-pyrimidinylamino)-2-(trifluoromethyl)phenyl]-3-methyl-1-piperazinyl}propionamide
N-{2-[(S)-2,6-dioxo-3-piperidyl]-1-oxo-5-isoindolinyl}(R)-2-{4-[2-chloro-4-(5-methyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-methyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(2,6-dimethyl-4-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-methyl-2-pyridylamino)phenyl]-1-piperazinyl}propionamide
N-{2-[(S)-2,6-dioxo-3-piperidyl]-1-oxo-5-isoindolinyl}{4-[2-chloro-4-(2-pyrimidinyloxy)phenyl]-1-piperazinyl}acetamide
N-{2-[(S)-2,6-dioxo-3-piperidyl]-1-oxo-5-isoindolinyl}[4-(2-chloro-4-cyanophenyl)-1-piperazinyl]acetamide
N-{2-[(S)-2,6-dioxo-3-piperidyl]-1-oxo-5-isoindolinyl}(R)-2-{(R)-3-methyl-4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{(R)-3-methyl-4-[4-(2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[4-(4,6-dimethyl-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-chloro-2-pyridylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[4-(N-methyl-N-2-pyrimidinylamino)-2-chlorophenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(4-chloro-2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[2-chloro-4-(6-methyl-4-pyrimidinylamino)phenyl]-1-piperazinyl}acetamide
N-{2-[(S)-2,6-dioxo-3-piperidyl]-1-oxo-5-isoindolinyl}[4-(2,4-dichlorophenyl)-1-piperazinyl]acetamide
N-{2-[(S)-2,6-dioxo-3-piperidyl]-1-oxo-5-isoindolinyl}{4-[2-chloro-4-(6-chloro-4-pyrimidinylamino)phenyl]-1-piperazinyl}acetamide
N-{2-[(S)-2,6-dioxo-3-piperidyl]-1-oxo-5-isoindolinyl}[4-(3,4-dichlorophenyl)-1-piperazinyl]acetamide
N-{2-[(S)-2,6-dioxo-3-piperidyl]-1-oxo-5-isoindolinyl}(R)-2-{4-[4-(2-pyrimidinylamino)-2-(trifluoromethyl)phenyl]-1-piperazinyl}butyramide
N-{2-[(S)-2,6-dioxo-3-piperidyl]-1-oxo-5-isoindolinyl}(S)-2-{3-[4-(2-pyridylamino)-2-(trifluoromethyl)phenyl]-1-azetidinyl}butyramide
N-{2-[(S)-2,6-dioxo-3-piperidyl]-1-oxo-5-isoindolinyl}(R)-2-{(R)-3-methyl-4-[4-(4-methyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[2-chloro-4-(4-methyl-2-pyridylamino)phenyl]-1-piperazinyl}butyramide
N-{2-[(S)-2,6-dioxo-3-piperidyl]-1-oxo-5-isoindolinyl}{3-[4-(N-methyl-N-4-pyrimidinylamino)-2-chlorophenyl]-1-azetidinyl}acetamide
N-{2-[(S)-2,6-dioxo-3-piperidyl]-1-oxo-5-isoindolinyl}(R)-2-{3-[4-(N-methyl-N-4-pyrimidinylamino)-2-chlorophenyl]-1-azetidinyl}propionamide
N-{2-[(S)-2,6-dioxo-3-piperidyl]-1-oxo-5-isoindolinyl}(R)-2-{4-[2-chloro-4-(4-methyl-2-pyridylamino)phenyl]-1-piperazinyl}butyramide
N-{2-[(S)-2,6-dioxo-3-piperidyl]-1-oxo-5-isoindolinyl}{3-[4-(N-methyl-N-2-pyrimidinylamino)-2-chlorophenyl]-1-azetidinyl}acetamide
N-{2-[(S)-2,6-dioxo-3-piperidyl]-1-oxo-5-isoindolinyl}(R)-2-{3-[4-(N-methyl-N-2-pyrimidinylamino)-2-chlorophenyl]-1-azetidinyl}propionamide
N-{2-[(S)-2,6-dioxo-3-piperidyl]-1-oxo-5-isoindolinyl}{4-[4-(1-azetidinyl)-2-(trifluoromethyl)phenyl]-1-piperazinyl}acetamide
N-{2-[(S)-2,6-dioxo-3-piperidyl]-1-oxo-5-isoindolinyl}{4-[2-(trifluoromethyl)-4-cumenyl]-1-piperazinyl}acetamide
N-{2-[(S)-2,6-dioxo-3-piperidyl]-1-oxo-5-isoindolinyl}{3-[4-(N-methyl-N-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}(R)-2-{3-[4-(N-methyl-N-2-pyrimidinylamino)-2-(trifluoromethyl)phenyl]-1-azetidinyl}propionamide
N-{2-[(S)-2,6-dioxo-3-piperidyl]-1-oxo-5-isoindolinyl}{4-[4-(4-isopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}(4-{4-[4-(1-pyrrolidinyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[2-chloro-4-(4-chloro-2-pyridylamino)phenyl]-1-piperazinyl}acetamide
N-{2-[(S)-2,6-dioxo-3-piperidyl]-1-oxo-5-isoindolinyl}{4-[4-(N-methyl-N-4-pyrimidinylamino)-2-chlorophenyl]-1-piperazinyl}acetamide
N-{2-[(S)-2,6-dioxo-3-piperidyl]-1-oxo-5-isoindolinyl}{4-[4-(5-isopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-morpholino-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[4-(N-methyl-N-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}(4-{4-[4-methyl-6-(trifluoromethyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[4-(4-cyclopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-chloro-2-(trifluoromethyl)phenyl]-1-piperazinyl}acetamide
N-{2-[(S)-2,6-dioxo-3-piperidyl]-1-oxo-5-isoindolinyl}(R)-2-{3-[4-(N-methyl-N-4-pyrimidinylamino)-2-(trifluoromethyl)phenyl]-1-azetidinyl}propionamide
N-{2-[(S)-2,6-dioxo-3-piperidyl]-1-oxo-5-isoindolinyl}(4-{4-[4-(tert-butyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[4-(N-methyl-N-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[2-chloro-4-(4-isopropyl-2-pyrimidinylamino)phenyl]-1-azetidinyl}acetamide
N-{2-[(S)-2,6-dioxo-3-piperidyl]-1-oxo-5-isoindolinyl}(3-{2-chloro-4-[4-(1-pyrrolidinyl)-2-pyrimidinylamino]phenyl}-1-azetidinyl)acetamide
N-{2-[(S)-2,6-dioxo-3-piperidyl]-1-oxo-5-isoindolinyl}{3-[2-chloro-4-(4-morpholino-2-pyrimidinylamino)phenyl]-1-azetidinyl}acetamide
N-{2-[(S)-2,6-dioxo-3-piperidyl]-1-oxo-5-isoindolinyl}(S)-2-{4-[4-(N-methyl-N-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{4-[4-(4,6-diaza-5-indanylamino)-2-(trifluoromethyl)phenyl]-1-piperazinyl}acetamide
N-{2-[(S)-2,6-dioxo-3-piperidyl]-1-oxo-5-isoindolinyl}{3-[2-chloro-4-(4-methyl-2-pyrimidinylamino)phenyl]-1-azetidinyl}acetamide
N-{2-[(S)-2,6-dioxo-3-piperidyl]-1-oxo-5-isoindolinyl}{3-[2-chloro-4-(4,6-dimethyl-2-pyrimidinylamino)phenyl]-1-azetidinyl}acetamide
N-[2-(2,6-dioxo-3-piperidyl)-1,3-dioxo-5-isoindolinyl]{3-[4-(N-methyl-N-2-pyrimidinylamino)-2-(trifluoromethyl)phenyl]-1-azetidinyl}acetamide
N-[2-(2,6-dioxo-3-piperidyl)-1,3-dioxo-5-isoindolinyl]{3-[4-(N-methyl-N-2-pyrimidinylamino)-2-chlorophenyl]-1-azetidinyl}acetamide
N-{2-[(S)-2,6-dioxo-3-piperidyl]-1-oxo-5-isoindolinyl}(S)-2-{4-[4-(N-methyl-N-4-pyrimidinylamino)-2-chlorophenyl]-1-piperazinyl}propionamide
N-{2-[(S)-2,6-dioxo-3-piperidyl]-1-oxo-5-isoindolinyl}(S)-2-{4-[4-(4,6-diaza-5-indanylamino)-2-(trifluoromethyl)phenyl]-1-piperazinyl}propionamide
N-{2-[(S)-2,6-dioxo-3-piperidyl]-1-oxo-5-isoindolinyl}(3-{4-[(6-chloro-4-pyrimidinyl)-N-methylamino]-2-(trifluoromethyl)phenyl}-1-azetidinyl)acetamide
N-{2-[(S)-2,6-dioxo-3-piperidyl]-1-oxo-5-isoindolinyl}(R)-2-(3-{4-[(6-chloro-4-pyrimidinyl)-N-methylamino]-2-(trifluoromethyl)phenyl}-1-azetidinyl)propionamide
N-{2-[(S)-2,6-dioxo-3-piperidyl]-1-oxo-5-isoindolinyl}2-{4-[4-(4,6-dimethyl-2-pyrimidinylamino)-2-(trifluoromethyl)phenyl]-1-piperazinyl}-2-methylpropionamide
N-{2-[(S)-2,6-dioxo-3-piperidyl]-1-oxo-5-isoindolinyl}(3-{4-[(4,6-dimethyl-2-pyrimidinyl)-N-methylamino]-2-(trifluoromethyl)phenyl}-1-azetidinyl)acetamide
N-{2-[(S)-2,6-dioxo-3-piperidyl]-1-oxo-5-isoindolinyl}{3-[4-chloro-2-(trifluoromethyl)phenyl]-1-azetidinyl}acetamide
N-{2-[(S)-2,6-dioxo-3-piperidyl]-1-oxo-5-isoindolinyl}2-{3-[2-chloro-4-(4,6-dimethyl-2-pyrimidinylamino)phenyl]-1-azetidinyl}-2-methylpropionamide
N-{2-[(S)-2,6-dioxo-3-piperidyl]-1-oxo-5-isoindolinyl}(4-{2-chloro-4-[(6-chloro-4-pyrimidinyl)-N-methylamino]phenyl}-1-piperazinyl)acetamide
N-{2-[(S)-2,6-dioxo-3-piperidyl]-1-oxo-5-isoindolinyl}(4-{4-[(4,6-dimethyl-2-pyrimidinyl)-N-methylamino]-2-(trifluoromethyl)phenyl}-1-piperazinyl)acetamide
N-{2-[(S)-2,6-dioxo-3-piperidyl]-1-oxo-5-isoindolinyl}(4-{4-[N-methyl(4-methyl-2-pyrimidinyl)amino]-2-(trifluoromethyl)phenyl}-1-piperazinyl)acetamide
N-{2-[(S)-2,6-dioxo-3-piperidyl]-1-oxo-5-isoindolinyl}(S)-2-(4-{4-[(4,6-dimethyl-2-pyrimidinyl)-N-methylamino]-2-(trifluoromethyl)phenyl}-1-piperazinyl)propionamide
N-{2-[(S)-2,6-dioxo-3-piperidyl]-1-oxo-5-isoindolinyl}(S)-2-(4-{4-[N-methyl(4-methyl-2-pyrimidinyl)amino]-2-(trifluoromethyl)phenyl}-1-piperazinyl)propionamide
N-{2-[(S)-2,6-dioxo-3-piperidyl]-1-oxo-5-isoindolinyl}[3-(2,4-dichlorophenyl)-1-azetidinyl]acetamide
N-{2-[(S)-2,6-dioxo-3-piperidyl]-1-oxo-5-isoindolinyl}{3-[2-chloro-4-(trifluoromethyl)phenyl]-1-azetidinyl}acetamide
N-{2-[(S)-2,6-dioxo-3-piperidyl]-1-oxo-5-isoindolinyl}(S)-2-{4-[4-(N-methyl-N-2-pyrimidinylamino)-2-(trifluoromethyl)phenyl]-1-piperazinyl}butyramide
N-{2-[(S)-2,6-dioxo-3-piperidyl]-1-oxo-5-isoindolinyl}(S)-2-(4-{4-[(4,6-dimethyl-2-pyrimidinyl)-N-methylamino]-2-(trifluoromethyl)phenyl}-1-piperazinyl)butyramide
N-{2-[(S)-2,6-dioxo-3-piperidyl]-1-oxo-5-isoindolinyl}{4-[4-(2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}1-{4-[4-(2-pyrimidinylamino)-2-(trifluoromethyl)phenyl]-1-piperazinyl}cyclopropanecarboxamide
2-[3-[4-[(6-chloropyrimidin-4-yl)amino]-2-(trifluoromethyl)phenyl]azetidin-1-yl]-N-[2-[(3S)-2,6-dioxo-3-piperidyl]-1-oxo-isoindolin-5-yl]acetamide
N-{2-[(S)-2,6-dioxo-3-piperidyl]-1-oxo-5-isoindolinyl}{3-[4-(2-methyl-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(4-methyl-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(6-methyl-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(4-chloro-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-methyl-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}1-{4-[4-(4,6-dimethyl-2-pyrimidinylamino)-2-(trifluoromethyl)phenyl]-1-piperazinyl}cyclopropanecarboxamide
N-{2-[(S)-2,6-dioxo-3-piperidyl]-1-oxo-5-isoindolinyl}(R)-2-{3-[4-(2-pyrimidinylamino)-2-(trifluoromethyl)phenyl]-1-azetidinyl}butyramide
N-{2-[(S)-2,6-dioxo-3-piperidyl]-1-oxo-5-isoindolinyl}{3-[4-(4-cyano-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(4,6-dimethyl-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-cyano-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-fluoro-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{4-[2,4-bis(trifluoromethyl)phenyl]-1-piperazinyl}acetamide
N-{2-[(S)-2,6-dioxo-3-piperidyl]-1-oxo-5-isoindolinyl}{3-[4-(6-fluoro-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-furyl)-2-(trifluoromethyl)phenyl]-1-azetidinyl}acetamide
N-{2-[(S)-2,6-dioxo-3-piperidyl]-1-oxo-5-isoindolinyl}{4-[4-(N-ethyl-N-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[4-(2-pyridyl)-2-(trifluoromethyl)phenyl]-1-azetidinyl}acetamide
N-{2-[(S)-2,6-dioxo-3-piperidyl]-1-oxo-5-isoindolinyl}(3-{4-[N-methyl(4-methyl-2-pyrimidinyl)amino]-2-(trifluoromethyl)phenyl}-1-azetidinyl)acetamide
N-{2-[(S)-2,6-dioxo-3-piperidyl]-1-oxo-5-isoindolinyl}(3-{4-[N-methyl(5-methyl-2-pyrimidinyl)amino]-2-(trifluoromethyl)phenyl}-1-azetidinyl)acetamide
N-{2-[(S)-2,6-dioxo-3-piperidyl]-1-oxo-5-isoindolinyl}{3-[4-(4,6-dimethyl-2-pyrimidinylamino)-2-(trifluoromethyl)phenyl]-1-pyrrolidinyl}acetamide
N-{2-[(S)-2,6-dioxo-3-piperidyl]-1-oxo-5-isoindolinyl}(4-{4-[N-methyl(6-methyl-4-pyrimidinyl)amino]-2-(trifluoromethyl)phenyl}-1-piperazinyl)acetamide
N-{2-[(S)-2,6-dioxo-3-piperidyl]-1-oxo-5-isoindolinyl}(4-{4-[(6-chloro-4-pyrimidinyl)-N-methylamino]-2-(trifluoromethyl)phenyl}-1-piperazinyl)acetamide
N-{2-[(S)-2,6-dioxo-3-piperidyl]-1-oxo-5-isoindolinyl}{3-[4-(2-pyrimidinylamino)-2-(trifluoromethyl)phenyl]-1-pyrrolidinyl}acetamide
N-{2-[(S)-2,6-dioxo-3-piperidyl]-1-oxo-5-isoindolinyl}(3-{4-[N-(²H₃)methyl-N-2-pyrimidinylamino]-2-(trifluoromethyl)phenyl}-1-azetidinyl)acetamide
N-{2-[(S)-2,6-dioxo-3-piperidyl]-1-oxo-5-isoindolinyl}1-{4-[4-(4-methyl-2-pyrimidinylamino)-2-(trifluoromethyl)phenyl]-1-piperazinyl}cyclopropanecarboxamide
N-{2-[(S)-2,6-dioxo-3-piperidyl]-1-oxo-5-isoindolinyl}[3-(4-bromo-2-trifluoromethoxyphenyl)-1-azetidinyl]acetamide
N-{2-[(S)-2,6-dioxo-3-piperidyl]-1-oxo-5-isoindolinyl}{3-[4-(2-pyrimidinylamino)-2-trifluoromethoxyphenyl]-1-azetidinyl}acetamide
N-{2-[(S)-2,6-dioxo-3-piperidyl]-1-oxo-5-isoindolinyl}{3-[4-(N-methyl-N-2-pyrimidinylamino)-2-trifluoromethoxyphenyl]-1-azetidinyl}acetamide
N-{2-[(S)-2,6-dioxo-3-piperidyl]-1-oxo-5-isoindolinyl}{4-[5-bromo-3-(trifluoromethyl)-2-pyridyl]-1-piperazinyl}acetamide
N-{2-[(S)-2,6-dioxo-3-piperidyl]-1-oxo-5-isoindolinyl}{4-[5-(N-methyl-N-2-pyrimidinylamino)-3-(trifluoromethyl)-2-pyridyl]-1-piperazinyl}acetamide
N-{2-[(S)-2,6-dioxo-3-piperidyl]-1-oxo-5-isoindolinyl}(4-{5-[(4-chloro-6-methyl-2-pyrimidinyl)-N-methylamino]-3-(trifluoromethyl)-2-pyridyl}-1-piperazinyl)acetamide
N-{2-[(S)-2,6-dioxo-3-piperidyl]-1-oxo-5-isoindolinyl}{4-[4-(2-pyrimidinylamino)-2-(trifluoromethyl)phenyl]-1,2,3,6-tetrahydro-1-pyridyl}acetamide
N-{2-[(S)-2,6-dioxo-3-piperidyl]-1-oxo-5-isoindolinyl}[3-(2-chloro-4-trifluoromethoxyphenyl)-1-azetidinyl]acetamide
N-{2-[(S)-2,6-dioxo-3-piperidyl]-1-oxo-5-isoindolinyl}{3-[4-fluoro-2-(trifluoromethyl)phenyl]-1-azetidinyl}acetamide
N-{2-[(S)-2,6-dioxo-3-piperidyl]-1-oxo-5-isoindolinyl}[3-(4-chloro-2-trifluoromethoxyphenyl)-1-azetidinyl]acetamide
N-{2-[(S)-2,6-dioxo-3-piperidyl]-1-oxo-5-isoindolinyl}{4-[4-(4-methyl-2-pyrimidinylamino)-2-(trifluoromethyl)phenyl]-1-piperidyl}acetamide
N-{2-[(S)-2,6-dioxo-3-piperidyl]-1-oxo-5-isoindolinyl}{6-[4-(2-pyrimidinylamino)-2-(trifluoromethyl)phenyl]-2,6-diaza-2-spiro[3.3]heptyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1-piperidyl}acetamide
N-{2-[(S)-2,6-dioxo-3-piperidyl]-1-oxo-5-isoindolinyl}{4-[3,5-bis(trifluoromethyl)-2-pyridyl]-1-piperazinyl}acetamide
N-{2-[(S)-2,6-dioxo-3-piperidyl]-1-oxo-5-isoindolinyl}1-(4-{4-[(4,6-dimethyl-2-pyrimidinyl)-N-(²H₃)methylamino]-2-(trifluoromethyl)phenyl}-1-piperazinyl)cyclopropanecarboxamide
N-{2-[(S)-2,6-dioxo-3-piperidyl]-1-oxo-5-isoindolinyl}[4-(2-chloro-4-trifluoromethoxyphenyl)-1-piperazinyl]acetamide
N-{2-[(S)-2,6-dioxo-3-piperidyl]-1-oxo-5-isoindolinyl}{3-[4-bromo-2-(trifluoromethyl)phenyl]-1-azetidinyl}acetamide
N-[2-(2,6-dioxo-3-piperidyl)-6-fluoro-1-oxo-5-isoindolinyl]{4-[4-(N-methyl-N-2-pyrimidinylamino)-2-(trifluoromethyl)phenyl]-1-piperazinyl}acetamide
N-[2-(2,6-dioxo-3-piperidyl)-6-fluoro-1-oxo-5-isoindolinyl]{4-[2,4-bis(trifluoromethyl)phenyl]-1-piperazinyl}acetamide
N-[2-(2,6-dioxo-3-piperidyl)-6-fluoro-1-oxo-5-isoindolinyl]{3-[2-chloro-4-(trifluoromethyl)phenyl]-1-azetidinyl}acetamide
N-{2-[(S)-2,6-dioxo-3-piperidyl]-1-oxo-5-isoindolinyl}(4-{5-[N-(²H₃)methyl-N-2-pyrimidinylamino]-3-(trifluoromethyl)-2-pyridyl}-1-piperazinyl)acetamide
N-{2-[(S)-2,6-dioxo-3-piperidyl]-1-oxo-5-isoindolinyl}{4-[4-(2-pyridyloxy)-2-(trifluoromethyl)phenyl]-1-piperazinyl}acetamide
N-{2-[(S)-2,6-dioxo-3-piperidyl]-1-oxo-5-isoindolinyl}{4-[4-(4,6-dimethyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{3-methyl-3-[4-(2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{4-[2-chloro-4-(4,6-dimethyl-2-pyrimidinyloxy)phenyl]-1-piperazinyl}acetamide
N-{2-[(S)-2,6-dioxo-3-piperidyl]-1-oxo-5-isoindolinyl}{4-[4-(4-methyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(4-methyl-2-pyrimidinyloxy)phenyl]-1-piperazinyl}acetamide
N-{2-[(S)-2,6-dioxo-3-piperidyl]-1-oxo-5-isoindolinyl}{4-[2-chloro-4-(6-methyl-4-pyrimidinyloxy)phenyl]-1-piperazinyl}acetamide
N-{2-[(S)-2,6-dioxo-3-piperidyl]-1-oxo-5-isoindolinyl}{4-[4-(6-methyl-4-pyrimidinyloxy)-2-(trifluoromethyl)phenyl]-1-piperazinyl}acetamide
N-{2-[(S)-2,6-dioxo-3-piperidyl]-6-fluoro-1-oxo-5-isoindolinyl}{4-[2,4-bis(trifluoromethyl)phenyl]-1-piperazinyl}acetamide
N-{2-[(S)-2,6-dioxo-3-piperidyl]-6-fluoro-1-oxo-5-isoindolinyl}{4-[2-chloro-4-(2-pyridylamino)phenyl]-1-piperazinyl}acetamide
N-{2-[(S)-2,6-dioxo-3-piperidyl]-6-fluoro-1-oxo-5-isoindolinyl}{4-[2-chloro-4-(4-methyl-2-pyrimidinyloxy)phenyl]-1-piperazinyl}acetamide
N-{2-[(S)-2,6-dioxo-3-piperidyl]-6-fluoro-1-oxo-5-isoindolinyl}{4-[2-chloro-4-(6-methyl-4-pyrimidinyloxy)phenyl]-1-piperazinyl}acetamide
N-{2-[(S)-2,6-dioxo-3-piperidyl]-6-fluoro-1-oxo-5-isoindolinyl}{4-[4-(4-methyl-2-pyrimidinyloxy)-2-(trifluoromethyl)phenyl]-1-piperazinyl}acetamide
N-[2-(2,6-dioxo-3-piperidyl)-4-fluoro-1-oxo-5-isoindolinyl]{4-[2-chloro-4-(2-pyridylamino)phenyl]-1-piperazinyl}acetamide
N-[2-(2,6-dioxo-3-piperidyl)-4-fluoro-1-oxo-5-isoindolinyl]{4-[2,4-bis(trifluoromethyl)phenyl]-1-piperazinyl}acetamide
N-[2-(2,6-dioxo-3-piperidyl)-4-fluoro-1-oxo-5-isoindolinyl]{4-[2-chloro-4-(6-methyl-4-pyrimidinyloxy)phenyl]-1-piperazinyl}acetamide
N-[2-(2,6-dioxo-3-piperidyl)-4-fluoro-1-oxo-5-isoindolinyl]{4-[2-chloro-4-(4-methyl-2-pyrimidinyloxy)phenyl]-1-piperazinyl}acetamide
N-[2-(2,6-dioxo-3-piperidyl)-4-fluoro-1-oxo-5-isoindolinyl]{4-[4-(4-methyl-2-pyrimidinyloxy)-2-(trifluoromethyl)phenyl]-1-piperazinyl}acetamide
N-{2-[(S)-2,6-dioxo-3-piperidyl]-6-fluoro-1-oxo-5-isoindolinyl}{3-[4-(2-pyrimidinyloxy)-2-(trifluoromethyl)phenyl]-1-azetidinyl}acetamide
N-{2-[(S)-2,6-dioxo-3-piperidyl]-6-fluoro-1-oxo-5-isoindolinyl}{3-[4-(6-methyl-4-pyrimidinyloxy)-2-(trifluoromethyl)phenyl]-1-azetidinyl}acetamide
N-{2-[(S)-2,6-dioxo-3-piperidyl]-6-fluoro-1-oxo-5-isoindolinyl}{3-[2-chloro-4-(6-methyl-4-pyrimidinyloxy)phenyl]-1-azetidinyl}acetamide
N-{2-[(S)-2,6-dioxo-3-piperidyl]-6-fluoro-1-oxo-5-isoindolinyl}{3-[2-chloro-4-(2-pyrimidinyloxy)phenyl]-1-azetidinyl}acetamide
N-{2-[(S)-2,6-dioxo-3-piperidyl]-1-oxo-5-isoindolinyl}{3-[2-chloro-4-(methylamino)phenyl]-1-azetidinyl}acetamide
N-{2-[(S)-2,6-dioxo-3-piperidyl]-1-oxo-5-isoindolinyl}{3-[4-(methylamino)-2-(trifluoromethyl)phenyl]-1-azetidinyl}acetamide
N-{2-[(R)-2,6-dioxo-3-piperidyl]-4-fluoro-1-oxo-5-isoindolinyl}{3-[2-chloro-4-(6-methyl-4-pyrimidinyloxy)phenyl]-1-azetidinyl}acetamide
N-{2-[(S)-2,6-dioxo-3-piperidyl]-4-fluoro-1-oxo-5-isoindolinyl}{3-[2-chloro-4-(2-pyrimidinyloxy)phenyl]-1-azetidinyl}acetamide
N-[2-(2,6-dioxo-3-piperidyl)-3-oxo-2,5-diaza-6-indanyl]{4-[2-chloro-4-(2-pyridylamino)phenyl]-1-piperazinyl}acetamide
N-[2-(2,6-dioxo-3-piperidyl)-1-oxo-2,4-diaza-5-indanyl]{4-[2-chloro-4-(2-pyridylamino)phenyl]-1-piperazinyl}acetamide
3-{5-[2-(4-{4-[5-(benzyloxy)-2-pyridylamino]-2-(trifluoromethyl)phenyl}-1-piperazinyl)ethoxy]-1-oxo-2-isoindolinyl}-2,6-piperidinedione
3-[5-(2-{4-[2,4-bis(trifluoromethyl)phenyl]-1-piperidyl}ethoxy)-1-oxo-2-isoindolinyl]-2,6-piperidinedione
3-[5-(2-{3-[2,4-bis(trifluoromethyl)phenyl]-1-azetidinyl}ethoxy)-1-oxo-2-isoindolinyl]-2,6-piperidinedione,

The present invention encompasses a pharmaceutically acceptable salt, diastereomer, enantiomer, racemate, hydrate, solvate, or prodrug of the above-listed compounds.

As used herein, the term “pharmaceutically acceptable” refers to a material, such as a carrier or diluent, which does not abrogate the biological activity, or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms include pharmaceutically acceptable acidic/anionic or basic/cationic salts (UK Journal of Pharmaceutical and Biosciences Vol. 2(4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include sodium, potassium, calcium, magnesium, diethanolamine, N-methyl-D-glucamine, L-lysine, L-arginine, ammonium, ethanolamine, piperazine and triethanolamine salts.

A pharmaceutically acceptable acid salt is formed by reaction of the free base form of a compound of Formula 1 with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid. A pharmaceutically acceptable acid addition salt of a compound of Formula 1 can comprise or be, for example, a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g., 2-naphthalenesulfonate) or hexanoate salt.

The free acid or free base forms of the compounds of the invention may be prepared by methods known to those of ordinary skill in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499; the entire teachings of which are incorporated herein by reference) from the corresponding base addition salt or acid addition salt form, respectively. For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Prodrug derivatives of the compounds of the invention may be prepared by methods known to those of ordinary skill in the art (e.g., for further details see Saulnier et al., Bioorg. Med. Chem. Letters, 1994, 4, 1985; Daniela Hartmann Jornada at. Al., Molecules 2016, 21, 42; the entire teachings of which are incorporated herein by reference). Protected derivatives of the compounds of the invention may be prepared by means known to those of ordinary skill in the art. A detailed description of techniques applicable to the creation of protecting groups and their removal can be found in T. W. Greene, “Green's Protective Groups in Organic Chemistry,” 4th edition, John Wiley and Sons, Inc., 2006, the entire teachings of which are incorporated herein by reference.

Compounds of the invention may be prepared as their individual stereoisomers by reaction of a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions,” John Wiley And Sons, Inc., 1981, the entire teachings of which are incorporated herein by reference.

As used herein, the term “solvate” refers to a complex of variable stoichiometry formed by a solute (in this invention, a compound of Formula 1 or a pharmaceutically acceptable salt thereof) and a solvent. Such solvents for the purpose of the invention may not interfere with the biological activity of the solute. Non-limiting examples of suitable solvents include water, acetone, methanol, ethanol, and acetic acid. Preferably the solvent used is a pharmaceutically acceptable solvent. Non-limiting examples of suitable pharmaceutically acceptable solvents include water, ethanol, and acetic acid.

Furthermore, the compounds of the invention may be prepared as crystalline forms. The crystalline forms may exist as polymorphs.

It should be noted that in view of the close relationship between the compound of the invention and their other forms, whenever a compound is referred to in this context herein, a corresponding salt, diastereomer, enantiomer, racemate, crystalline, polymorph, prodrug, hydrate, or solvate is also intended, if it is possible or appropriate under certain circumstances.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

The term “about” is used in this disclosure to allow for a degree of variability in a value or range, for example, within 10%, within 5%, or within 1% of a stated value or of a stated limit of a range.

The term “substantially” used in this disclosure to allow for a degree of variability in a value or range, for example, within 90%, within 95%, or within 99% of a stated value or of a stated limit of a range.

The term “alkyl,” whether used alone or as part of a larger moiety such as “arylalkyl,” “haloalkyl,” or “cycloalkyl,” refers to a straight or branched hydrocarbon radical having from one to six carbon atoms (unless otherwise specified) and includes, for example, methyl, ethyl, n-propyl, iso-propyl, n-butyl, sec-butyl, tert-butyl, n-pentyl, iso-pentyl, n-hexyl, —CF₃, —CH₂CF₃, and the like. An alkyl group may be unsubstituted or substituted with one or more suitable substituents.

The term “cycloalkyl” refers to a monocyclic or polycyclic hydrocarbon ring group having from 3 to 8 carbon atoms in the hydrocarbon ring (unless stated otherwise) and includes, for example, cyclopropyl, cycloheptyl, cyclooctyl, cyclodecyl, cyclobutyl, adamantyl, norpinanyl, decalinyl, norbornyl, cyclohexyl, cyclopentyl, and the like. A cycloalkyl group can be unsubstituted or substituted with one or more suitable substituents.

The term “hetero” refers to the replacement of at least one carbon atom member in a ring system with at least one heteroatom such as nitrogen, sulfur, sulfoxide, sulfone, and oxygen.

As used herein, the term “halo” or “halogen” includes fluoro (or fluorine), chloro (or chlorine), bromo (or bromine), and iodo (or iodine).

As used herein, the term “alkoxy” refers to the alkyl groups as defined above bound through oxygen, examples of which include methoxy, ethoxy, iso-propoxy, tert-butoxy, fluorinated C1-4 alkoxy, such as —OCF₃, —OCHF₂, and the like. In addition, the term also refers to polyethers such as —O—(CH₂)₂O—CH₃, and the like. An alkoxy group can be unsubstituted or substituted with one or more suitable substituents.

As used herein, the term “haloalkoxy” refers to an alkoxy group in which one or more hydrogen atoms on the alkyl moiety are replaced by halogen atoms such as fluorine, chlorine, bromine, or iodine. Non-limiting examples of haloalkoxy groups include fluoroalkoxy, chloroalkoxy, trifluoromethoxy, difluoromethoxy, trifluoroethoxy, perfluoropropoxy, and partially fluorinated propoxy groups. The term “C₁₋₆ haloalkoxy” refers to haloalkoxy groups having one to six carbon atoms, wherein one or more hydrogen atoms on the alkyl chain are replaced by halogen atoms. Non-limiting examples of C₁₋₆ haloalkoxy groups include —OCF₃, —OCH₂CF₃, and —OCHFCHCF₂.

The term “C₁₋₆ haloalkoxy-substituted alkyl” or “haloalkoxy-substituted moiety” as used herein is intended to encompass structures wherein a haloalkoxy group is attached through a carbon or heteroatom linkage, such as —O(CH₂)ₙC₁₋₆ cycloalkylNR₆ₐR_{6β}, unless otherwise specified. Accordingly, compounds containing an —O(CH₂)ₙ-cycloalkyl-NR— or equivalent motif are considered to fall within the scope of the definitions provided herein. As used herein, the substituent “—O(CH₂)ₙC₁₋₆ cycloalkylNR₆ₐR_{6β}” is intended to encompass linkages wherein an alkoxy moiety is connected to a cycloalkyl ring bearing a nitrogen-containing substituent. Such substituents may occur at R₁, R₂, or R₃ positions, depending on the embodiment, and are considered within the scope of the defined substituents including, but not limited to, haloalkoxy-substituted moieties.

As used herein, the term “aryl” refers to unsubstituted or substituted aromatic monocyclic or polycyclic groups and includes, for example, carbocyclic aromatic groups such as phenyl, naphthyl and the like, as well as heteroaromatic groups such as pyridyl, furanyl, thiophenyl, and the like. The term “aryl” also includes an aromatic ring (such as a phenyl or pyridyl ring) fused to a non-aromatic carbocyclic or heterocyclic ring. The term “aryl” may be interchangeably used with “aryl ring,” aromatic group,” and “aromatic ring.” Heteroaryl groups have 4 to 14 atoms in the heteroaromatic ring(s), 1 to 9 of which are independently selected from the group consisting of oxygen, sulfur and nitrogen. Heteroaryl groups have 1-3 heteroatoms in a 5-8 membered aromatic group. An aryl or heteroaryl can be a mono- or bicyclic aromatic group. Typical aryl and heteroaryl groups include, for example, phenyl, quinolinyl, indazoyl, indolyl, dihydrobenzodioxynyl, 3-chlorophenyl, 2,6-dibromophenyl, pyridyl, pyrimidinyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, pyrrole, pyrazole, imidazole, thiazole, and the like. An aryl or heteroaryl group can be unsubstituted or substituted with one or more suitable substituents.

As used herein, the term “hydroxyl” or “hydroxy” refers to —OH.

As used herein, the term “nitro” refers to —NO₂

As used herein, the term “thiol” or “thioly” refers to —SH.

As used herein, the term “cyano” or “nitrile” refers to —CN

As used herein, the term “amino” or “amine” refers to —NH₂.

As used herein, the term “alkylOH” refers to any hydroxyl derivative of alkyl radical. The term “alkylOH” includes any alkyl radical having one or more hydrogen atoms replaced by a hydroxy group.

As used herein, the term “alkylNH₂” refers to any amine (primary, secondary, or tertiary amine) derivative of alkyl radical. The term “alkylNH₂” includes any alkyl radical having one or more hydrogen atoms replaced by an amine (primary amine) or substituted amine (secondary or tertiary amine) group.

It is further understood that the abbreviations “C” and “N” are representative for all possible degrees of saturation, which typically do not result in radicals, nitrenes or carbenes, i.e. N includes —NH— and —N═, C includes —CH₂— and ═CH—. In addition, “C” as an atom in an aromatic or heteroaromatic ring which has a substituent R_{XX} at any suitable position, includes ═CH— as well as ═CR_{XX}—.

By “optional” or “optionally,” it means that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment, an optionally substituted group has 2 substituents. In another embodiment, an optionally substituted group has 3 substituents. In another embodiment, an optionally substituted group has 4 substituents. In another embodiment, an optionally substituted group has 5 substituents. For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bonded to a halogen atom, a hydroxyl group, or any other substituent described herein. Thus, the term “optionally substituted” means that a given chemical moiety has the potential to contain other functional groups, but it does not necessarily have any further functional groups.

The compounds of the present invention may be screened using global proteomics to access their degradation of the activity on at least one CRBN neo-substrate. Examples of such proteins include, but are not limited to, GSPT1 and GSPT2.

### 2. Compositions

In another aspect, the present invention provides a pharmaceutical composition comprising one or more components selected from the group consisting of a compound, a salt, a diastereomer, an enantiomer, a racemate, a hydrate, a solvate, and a prodrug in accordance with the present invention. In some embodiments, the composition may further comprise one or more additional components, which may be chemically or biologically active or inactive. Non-limiting examples of the additional components may include a pharmaceutically acceptable carrier, a stabilizer, a dispersing agent, a suspending agent, a thickening agent, a diluent, an excipient, and an adjuvant. In some embodiments, the composition may further comprise at least one additional active agent as a combination therapy.

As used herein, the term “composition” is intended to encompass a product comprising the compound, salt, diastereomer, enantiomer, racemate, hydrate, solvate, or a pharmaceutical combination thereof in the therapeutically effective amount. Also, the term should be interpreted to encompass any other product which results, directly or indirectly, from claimed compound, salt, diastereomer, enantiomer, racemate, hydrate, solvate, or a pharmaceutical combination thereof.

As used herein, the term “pharmaceutical composition” refers to a mixture of a therapeutically active component (ingredient) with one or more other components, which may be chemically or biologically active or inactive.

As used herein, the term “pharmaceutical combination” means a product that results from the mixing or combining of more than one active ingredient.

As used herein, the term “acceptable” with respect to a formulation, composition, or ingredient, as used herein, means having no persistent detrimental effect on the general health of the subject being treated.

As used herein, the term “carrier” refers to chemical compounds or agents that facilitate the incorporation of a compound described herein into cells or tissues.

As used herein, the term “diluent” refers to chemical compounds that are used to dilute a compound described herein prior to delivery. Diluents can also be used to stabilize compounds described herein.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein.

The compositions described herein may be in the form of solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or -free inhale solutions). Also, the compositions described herein may be formulated for sustained or slow release.

### 3. Methods of Using Compounds or Compositions

In still another aspect, the present invention provides methods of treating cancer, which comprises administering to a subject or patient in need a therapeutically effective amount of the composition described herein, wherein the composition degrades a target protein via cereblon, thereby decreasing GSPT1, MYC, or both.

In some embodiments, the present invention provides methods of treating or alleviating certain GSPT-mediated diseases or conditions by administering a subject or patient a therapeutically effective amount of the composition described herein. In some embodiments, the present invention provides methods of treating or alleviating certain GSPT-mediated diseases or disorders in which GSPT is known to play a role.

In some embodiments, the present invention provides methods of inhibiting the proliferation of tumor cells through degradation activities, including targeting GSPT, global proteomics, and xenograft, by administering a subject or patient a therapeutically effective amount of the composition described herein.

Also provided are pharmaceutical compositions formulated for administration by an appropriate route, containing effective concentrations of one or more of the compounds provided herein, or an enantiomer or a mixture of enantiomers thereof, or a pharmaceutically acceptable salt, solvate, hydrate, co-crystal, clathrate, or polymorph thereof, and optionally comprising at least one pharmaceutical carrier.

In some embodiments, the pharmaceutical compositions deliver amounts effective for the treatment of cancer, including solid tumors and blood-borne tumors associated with GSPT1, with particular emphasis on cancers where overexpression of GSPT1 is known to play pathogenic roles. These include, but are not limited to, solid cancers such as those of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastasis, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

In addition, the pharmaceutical compositions are effective for the treatment of blood-borne (liquid) or hematological cancers, including, but not limited to, leukemias, lymphomas, and myelomas, such as diffuse large B-cell lymphoma (DLBCL), B cell immunoblastic lymphoma, small non-cleaved cell lymphoma, human lymphotropic virus type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Also provided herein are combination therapies using one or more compounds or compositions provided herein, or an enantiomer or a mixture of enantiomers thereof, or a diastereomer or a mixture of diastereomers thereof, or a pharmaceutically acceptable salt, solvate, hydrate, co-crystal, clathrate, or polymorph thereof in combination with a therapy such as another pharmaceutical agent with activity against cancer or its symptoms. Examples of combination therapies within the scope of the methods include, but are not limited to, surgery, chemotherapy, radiation therapy, hormonal therapy, biological therapy, immunotherapy, and combinations thereof.

The compounds or compositions provided herein, or pharmaceutically acceptable derivatives thereof may be administered simultaneously with, before, or after administration of one or more of the therapies mentioned above. Pharmaceutical compositions containing a compound provided herein and one or more of the above agents are also provided.

In certain embodiments, the compounds of the present invention may be included in an antibody drug conjugate. Antibody-drug conjugates (ADCs) or degrader antibody conjugates (DACs) are highly targeted biopharmaceutical drugs that combine monoclonal antibodies, specific to surface antigens present on tumor cells, with highly potent anti-cancer agents linked via a chemical, cleavable, or non-cleavable linker.

In certain embodiments, provided herein are methods of treating, preventing or ameliorating cancer, including solid tumors and blood-borne tumors, or one or more symptoms or causes thereof. In certain embodiments, the solid tumor is lung cancer. The methods provided herein may encompass the treatment of various forms of lung cancer, including large cell lung cancer, squamous cell carcinoma, small-cell lung cancer, and adenocarcinoma.

In certain embodiments, the methods provided herein may encompass the prevention or management of disease progression in various forms of lung cancer, including large cell lung cancer, squamous cell carcinoma, small-cell lung cancer, and lung adenocarcinoma.

The methods provided herein may include treatment or prevention of metastasis of solid tumors, such as lung cancers, hepatocellular carcinoma cells, breast cancer, and colon cancers, that are driven by the MYC and WNT signaling pathway.

The methods provided herein may include the treatment, and management of leukemias that are relapsed, refractory or resistant.

In some embodiments, the methods provided herein may encompass the treatment and/or regression and/or management of solid tumors and blood cancers.

In practicing the methods, therapeutically effective amounts of the compounds or compositions may be administered to an individual exhibiting the symptoms of the disease or disorder to be treated. The amounts are sufficient to ameliorate or eliminate one or more symptoms of the disease or disorder.

In some embodiments, the present invention may provide a pharmaceutical pack or kit comprising one or more containers filled with one or more ingredients of the pharmaceutical compositions. Optionally, such container(s) may be accompanied by a notice in the form prescribed by a governmental agency regulating the manufacture, use, or sale of pharmaceuticals or biological products, reflecting the agency's approval for human administration. The pack or kit can also be labeled with information regarding the mode of administration, the sequence of drug administration (e.g., separately, sequentially, or concurrently), or other relevant details.

As used herein, the term “subject” or “patient” encompasses mammals and non-mammals. Examples of mammals include, but are not limited to, humans, chimpanzees, apes, monkeys, cattle, horses, sheep, goats, swine, rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or prodrugs thereof to a subject in need of treatment.

As used herein, the term “effective amount” or “therapeutically effective amount” refers to the sufficient amount of a compound described herein that, when administered, relieves to some extent one or more of the symptoms of the disease or condition being treated. The result may be a reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques such as a dose escalation study. By way of example only, a therapeutically effective amount of a compound of the invention may range from about 0.01 mg/kg/day to about 1000 mg/kg/day, from about 0.1 mg/kg/day to about 500 mg/kg/day, or from about 0.1 mg (×2)/kg/day to about 500 mg (×2)/kg/day.

In addition, the compounds or compositions described herein may be administered singly or in combination with one or more additional therapeutic agents. The methods of administration of such compounds and compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration, optic administration, or transdermal administration.

The compounds or compositions provided herein may be administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops, ear drops, or patches.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

### 4. Methods of Preparing Compounds

In a further aspect, the present invention provides methods of preparing the compounds, salts, diastereomers, enantiomers, racemates, hydrates, solvates, and prodrugs. In some embodiments, the compounds, salts, diastereomers, enantiomers, racemates, hydrates, solvates, or prodrugs may be prepared by methods including, but not limited to, one or more of the following methods:
a. optionally converting a compound of the invention into a pharmaceutically acceptable salt;
b. optionally converting a salt form of a compound of the invention to a non-salt form;
c. optionally converting an unoxidized form of a compound of the invention into a pharmaceutically acceptable N-oxide;
d. optionally resolving an individual isomer of a compound of the invention from a mixture of stereoisomers;
e. optionally converting a non-derivatized compound of the invention into a pharmaceutically acceptable prodrug derivative; and
f. optionally converting a prodrug derivative of a compound of the invention to its non-derivatized form.

Exemplary methods for preparing the compounds of the invention are described herein, including in the Examples, which will be described in detail below. Some embodiments of the invention provide processes for preparing the compounds of the present invention, as illustrated in General Schemes 1-31.

In certain embodiments, the invention provides methods for preparing compounds represented by Formula (I).

Representative compounds of Formula (I) are listed below.
N-{2-[(S)-2,6-dioxo-3-piperidyl]-1-oxo-5-isoindolinyl}[3-(p-bromophenyl)-1-azetidinyl]acetamide
N-{2-[(S)-2,6-dioxo-3-piperidyl]-1-oxo-5-isoindolinyl}[4-(o-fluorophenyl)-1-piperazinyl]acetamide
N-{2-[(S)-2,6-dioxo-3-piperidyl]-1-oxo-5-isoindolinyl}[4-(o-methoxyphenyl)-1-piperazinyl]acetamide
N-{2-[(S)-2,6-dioxo-3-piperidyl]-1-oxo-5-isoindolinyl}{4-[2-methoxy-4-(2-pyridylamino)phenyl]-1-piperazinyl}acetamide
N-{2-[(S)-2,6-dioxo-3-piperidyl]-1-oxo-5-isoindolinyl}{4-[4-(2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(2-pyridylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}propionamide
N-{2-[(S)-2,6-dioxo-3-piperidyl]-1-oxo-5-isoindolinyl}2-{4-[2-methoxy-4-(2-pyridylamino)phenyl]-1-piperazinyl}propionamide
N-{2-[(S)-2,6-dioxo-3-piperidyl]-1-oxo-5-isoindolinyl}2-{4-[4-(2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[4-(6-chloro-4-pyrimidinylamino)-2-fluorophenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(6-chloro-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(2-pyrimidinylamino)phenyl]-1-piperazinyl}acetamide
N-{2-[(S)-2,6-dioxo-3-piperidyl]-1-oxo-5-isoindolinyl}{4-[4-amino-2-(trifluoromethyl)phenyl]-1-piperazinyl}acetamide
N-{2-[(S)-2,6-dioxo-3-piperidyl]-1-oxo-5-isoindolinyl}{4-[4-(4-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[p-(2-pyridylamino)phenyl]-1-azetidinyl}acetamide
N-{2-[(S)-2,6-dioxo-3-piperidyl]-1-oxo-5-isoindolinyl}2-{3-[p-(2-pyridylamino)phenyl]-1-azetidinyl}propionamide
N-{2-[(S)-2,6-dioxo-3-piperidyl]-1-oxo-5-isoindolinyl}{4-[2-methoxy-4-(2-pyrimidinylamino)phenyl]-1-piperazinyl}acetamide
N-{2-[(S)-2,6-dioxo-3-piperidyl]-1-oxo-5-isoindolinyl}{4-[2-chloro-4-(4-pyrimidinylamino)phenyl]-1-piperazinyl}acetamide
N-{2-[(S)-2,6-dioxo-3-piperidyl]-1-oxo-5-isoindolinyl}{4-[3-methoxy-4-(2-pyrimidinylamino)phenyl]-1-piperazinyl}acetamide
N-{2-[(S)-2,6-dioxo-3-piperidyl]-1-oxo-5-isoindolinyl}{4-[3-methoxy-4-(2-pyridylamino)phenyl]-1-piperazinyl}acetamide
N-{2-[(S)-2,6-dioxo-3-piperidyl]-1-oxo-5-isoindolinyl}{4-[2-fluoro-4-(2-pyridylamino)phenyl]-1-piperazinyl}acetamide
N-{2-[(S)-2,6-dioxo-3-piperidyl]-1-oxo-5-isoindolinyl}{4-[2-fluoro-4-(2-pyrimidinylamino)phenyl]-1-piperazinyl}acetamide
N-{2-[(S)-2,6-dioxo-3-piperidyl]-1-oxo-5-isoindolinyl}{4-[2-fluoro-4-(4-pyrimidinylamino)phenyl]-1-piperazinyl}acetamide
N-{2-[(S)-2,6-dioxo-3-piperidyl]-1-oxo-5-isoindolinyl}{4-[p-(2-pyridylamino)phenyl]-1-piperazinyl}acetamide
N-{2-[(S)-2,6-dioxo-3-piperidyl]-1-oxo-5-isoindolinyl}{4-[p-(2-pyrimidinylamino)phenyl]-1-piperazinyl}acetamide
N-{2-[(S)-2,6-dioxo-3-piperidyl]-1-oxo-5-isoindolinyl}{4-[4-(2-pyridylamino)-2-tolyl]-1-piperazinyl}acetamide
N-{2-[(S)-2,6-dioxo-3-piperidyl]-1-oxo-5-isoindolinyl}{4-[4-(2-pyrimidinylamino)-2-tolyl]-1-piperazinyl}acetamide
N-{2-[(S)-2,6-dioxo-3-piperidyl]-1-oxo-5-isoindolinyl}{4-[4-(6-chloro-4-pyrimidinylamino)-2-tolyl]-1-piperazinyl}acetamide
N-{2-[(S)-2,6-dioxo-3-piperidyl]-1-oxo-5-isoindolinyl}(S)-2-{4-[2-chloro-4-(2-pyridylamino)phenyl]-1-piperazinyl}propionamide
N-{2-[(S)-2,6-dioxo-3-piperidyl]-1-oxo-5-isoindolinyl}(R)-2-{4-[2-chloro-4-(2-pyridylamino)phenyl]-1-piperazinyl}propionamide
N-{2-[(S)-2,6-dioxo-3-piperidyl]-1-oxo-5-isoindolinyl}{4-[4-(4,6-dimethyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}2-{4-[2-chloro-4-(2-pyrimidinylamino)phenyl]-1-piperazinyl}propionamide
N-{2-[(S)-2,6-dioxo-3-piperidyl]-1-oxo-5-isoindolinyl}2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}2-{4-[4-(6-chloro-4-pyrimidinylamino)-2-tolyl]-1-piperazinyl}propionamide
N-{2-[(S)-2,6-dioxo-3-piperidyl]-1-oxo-5-isoindolinyl}{4-[2,6-difluoro-4-(2-pyrimidinylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}butyramide
N-{2-[(S)-2,6-dioxo-3-piperidyl]-1-oxo-5-isoindolinyl}{4-[2-chloro-4-(4,6-dimethyl-2-pyrimidinylamino)phenyl]-1-piperazinyl}acetamide
N-{2-[(S)-2,6-dioxo-3-piperidyl]-1-oxo-5-isoindolinyl}{4-[4-(4,6-dimethyl-2-pyrimidinylamino)-2-methoxyphenyl]-1-piperazinyl}acetamide
N-{2-[(S)-2,6-dioxo-3-piperidyl]-1-oxo-5-isoindolinyl}(R)-2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{4-[2-cyano-4-(4,6-dimethyl-2-pyrimidinylamino)phenyl]-1-piperazinyl}acetamide
N-{2-[(S)-2,6-dioxo-3-piperidyl]-1-oxo-5-isoindolinyl}{4-[4-(4-cyano-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(6-methyl-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}(R)-2-{4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{3-oxo-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{(R)-2-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-cyano-4-(2-pyrimidinylamino)phenyl]-1-piperazinyl}acetamide
N-{2-[(S)-2,6-dioxo-3-piperidyl]-1-oxo-5-isoindolinyl}(S)-2-{4-[2-cyano-4-(2-pyrimidinylamino)phenyl]-1-piperazinyl}propionamide
N-{2-[(S)-2,6-dioxo-3-piperidyl]-1-oxo-5-isoindolinyl}{(S)-2-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{(R)-3-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methoxy-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[4-(4-methoxy-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{(S)-3-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}[4-(4-amino-2-chlorophenyl)-1-piperazinyl]acetamide
N-{2-[(S)-2,6-dioxo-3-piperidyl]-1-oxo-5-isoindolinyl}{4-[4-(4,6-dimethyl-2-pyrimidinylamino)-2-tolyl]-1-piperazinyl}acetamide
N-{2-[(S)-2,6-dioxo-3-piperidyl]-1-oxo-5-isoindolinyl}{4-[4-(2,6-dimethyl-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[2-methoxy-4-(2-pyrimidinylamino)phenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyrimidinylamino)-2-methoxyphenyl]-1-piperazinyl}propionamide
N-{2-[(S)-2,6-dioxo-3-piperidyl]-1-oxo-5-isoindolinyl}{4-[4-(5-methyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methoxy-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1,4-diazepan-1-yl}acetamide
N-{2-[(S)-2,6-dioxo-3-piperidyl]-1-oxo-5-isoindolinyl}{4-[4-(methylamino)-2-(trifluoromethyl)phenyl]-1-piperazinyl}acetamide
N-{2-[(S)-2,6-dioxo-3-piperidyl]-1-oxo-5-isoindolinyl}{4-[4-(dimethylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(5-methyl-2-pyrimidinylamino)phenyl]-1-piperazinyl}acetamide
N-{2-[(S)-2,6-dioxo-3-piperidyl]-1-oxo-5-isoindolinyl}{4-[2-chloro-4-(methylamino)phenyl]-1-piperazinyl}acetamide
N-{2-[(S)-2,6-dioxo-3-piperidyl]-1-oxo-5-isoindolinyl}{4-[2-chloro-4-(dimethylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}-2-methylpropionamide
N-{2-[(S)-2,6-dioxo-3-piperidyl]-1-oxo-5-isoindolinyl}{3-[2-chloro-4-(2-pyridylamino)phenyl]-1-azetidinyl}acetamide
N-{2-[(S)-2,6-dioxo-3-piperidyl]-1-oxo-5-isoindolinyl}{3-[2-chloro-4-(4-methyl-2-pyridylamino)phenyl]-1-azetidinyl}acetamide
N-{2-[(S)-2,6-dioxo-3-piperidyl]-1-oxo-5-isoindolinyl}(S)-2-{4-[2-chloro-4-(4,6-dimethyl-2-pyrimidinylamino)phenyl]-1-piperazinyl}propionamide
N-{2-[(S)-2,6-dioxo-3-piperidyl]-1-oxo-5-isoindolinyl}{4-[2-chloro-4-(4-methyl-2-pyrimidinylamino)phenyl]-1-piperazinyl}acetamide
N-{2-[(S)-2,6-dioxo-3-piperidyl]-1-oxo-5-isoindolinyl}{4-[2-chloro-4-(2,6-dimethyl-4-pyrimidinylamino)phenyl]-1-piperazinyl}acetamide
N-{2-[(S)-2,6-dioxo-3-piperidyl]-1-oxo-5-isoindolinyl}{3-[4-(4-methyl-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[2-chloro-4-(2-pyrimidinylamino)phenyl]-1-azetidinyl}acetamide
N-{2-[(S)-2,6-dioxo-3-piperidyl]-1-oxo-5-isoindolinyl}{4-[2-chloro-4-(4-cyano-2-pyridylamino)phenyl]-1-piperazinyl}acetamide
N-{2-[(S)-2,6-dioxo-3-piperidyl]-1-oxo-5-isoindolinyl}{3-[4-(4,6-dimethyl-2-pyrimidinylamino)-2-(trifluoromethyl)phenyl]-3,6-diazabicyclo[3.1.1]hept-6-yl}acetamide
N-{2-[(S)-2,6-dioxo-3-piperidyl]-1-oxo-5-isoindolinyl}{(R)-3-methyl-4-[4-nitro-2-(trifluoromethyl)phenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-4,7-diaza-7-spiro[2.5]octyl}acetamide
N-{2-[(S)-2,6-dioxo-3-piperidyl]-1-oxo-5-isoindolinyl}{4-[4-(4-methyl-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(4-methyl-2-pyridylamino)phenyl]-1-piperazinyl}acetamide
N-{2-[(S)-2,6-dioxo-3-piperidyl]-1-oxo-5-isoindolinyl}{3-[4-(2-pyrimidinylamino)-2-(trifluoromethyl)phenyl]-3,6-diazabicyclo[3.1.1]hept-6-yl}acetamide
N-{2-[(S)-2,6-dioxo-3-piperidyl]-1-oxo-5-isoindolinyl}{(S)-4-[4-amino-2-(trifluoromethyl)phenyl]-3-methyl-1-piperazinyl}acetamide
N-{2-[(S)-2,6-dioxo-3-piperidyl]-1-oxo-5-isoindolinyl}{(R)-4-[4-(4,6-dimethyl-2-pyrimidinylamino)-2-(trifluoromethyl)phenyl]-3-methyl-1-piperazinyl}acetamide
N-{2-[(S)-2,6-dioxo-3-piperidyl]-1-oxo-5-isoindolinyl}{(R)-4-[4-(4-cyano-2-pyridylamino)-2-(trifluoromethyl)phenyl]-3-methyl-1-piperazinyl}acetamide
N-{2-[(S)-2,6-dioxo-3-piperidyl]-1-oxo-5-isoindolinyl}(R)-2-{4-[2-chloro-4-(4,6-dimethyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-cyano-2-pyridylamino)phenyl]-1-piperazinyl}propionamide
N-{2-[(S)-2,6-dioxo-3-piperidyl]-1-oxo-5-isoindolinyl}(R)-2-{4-[4-(2,6-dimethyl-4-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(5-methyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(4-methyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(4-methyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(R)-2-{3-[4-(2-pyrimidinylamino)-2-(trifluoromethyl)phenyl]-1-azetidinyl}propionamide
N-{2-[(S)-2,6-dioxo-3-piperidyl]-1-oxo-5-isoindolinyl}(R)-2-{3-[2-chloro-4-(2-pyrimidinylamino)phenyl]-1-azetidinyl}propionamide
N-{2-[(S)-2,6-dioxo-3-piperidyl]-1-oxo-5-isoindolinyl}{4-[4-(N-methyl-N-2-pyrimidinylamino)-2-chlorophenyl]-1-piperazinyl}acetamide
N-{2-[(S)-2,6-dioxo-3-piperidyl]-1-oxo-5-isoindolinyl}[4-(4-bromo-2-chlorophenyl)-1-piperazinyl]acetamide
N-{2-[(S)-2,6-dioxo-3-piperidyl]-1-oxo-5-isoindolinyl}(R)-2-{(R)-4-[4-(4,6-dimethyl-2-pyrimidinylamino)-2-(trifluoromethyl)phenyl]-3-methyl-1-piperazinyl}propionamide
N-{2-[(S)-2,6-dioxo-3-piperidyl]-1-oxo-5-isoindolinyl}(R)-2-{4-[2-chloro-4-(5-methyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-methyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(2,6-dimethyl-4-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-methyl-2-pyridylamino)phenyl]-1-piperazinyl}propionamide
N-{2-[(S)-2,6-dioxo-3-piperidyl]-1-oxo-5-isoindolinyl}{4-[2-chloro-4-(2-pyrimidinyloxy)phenyl]-1-piperazinyl}acetamide
N-{2-[(S)-2,6-dioxo-3-piperidyl]-1-oxo-5-isoindolinyl}[4-(2-chloro-4-cyanophenyl)-1-piperazinyl]acetamide
N-{2-[(S)-2,6-dioxo-3-piperidyl]-1-oxo-5-isoindolinyl}(R)-2-{(R)-3-methyl-4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{(R)-3-methyl-4-[4-(2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[4-(4,6-dimethyl-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-chloro-2-pyridylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[4-(N-methyl-N-2-pyrimidinylamino)-2-chlorophenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(4-chloro-2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[2-chloro-4-(6-methyl-4-pyrimidinylamino)phenyl]-1-piperazinyl}acetamide
N-{2-[(S)-2,6-dioxo-3-piperidyl]-1-oxo-5-isoindolinyl}[4-(2,4-dichlorophenyl)-1-piperazinyl]acetamide
N-{2-[(S)-2,6-dioxo-3-piperidyl]-1-oxo-5-isoindolinyl}{4-[2-chloro-4-(6-chloro-4-pyrimidinylamino)phenyl]-1-piperazinyl}acetamide
N-{2-[(S)-2,6-dioxo-3-piperidyl]-1-oxo-5-isoindolinyl}[4-(3,4-dichlorophenyl)-1-piperazinyl]acetamide
N-{2-[(S)-2,6-dioxo-3-piperidyl]-1-oxo-5-isoindolinyl}(R)-2-{4-[4-(2-pyrimidinylamino)-2-(trifluoromethyl)phenyl]-1-piperazinyl}butyramide
N-{2-[(S)-2,6-dioxo-3-piperidyl]-1-oxo-5-isoindolinyl}(S)-2-{3-[4-(2-pyridylamino)-2-(trifluoromethyl)phenyl]-1-azetidinyl}butyramide
N-{2-[(S)-2,6-dioxo-3-piperidyl]-1-oxo-5-isoindolinyl}(R)-2-{(R)-3-methyl-4-[4-(4-methyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[2-chloro-4-(4-methyl-2-pyridylamino)phenyl]-1-piperazinyl}butyramide
N-{2-[(S)-2,6-dioxo-3-piperidyl]-1-oxo-5-isoindolinyl}{3-[4-(N-methyl-N-4-pyrimidinylamino)-2-chlorophenyl]-1-azetidinyl}acetamide
N-{2-[(S)-2,6-dioxo-3-piperidyl]-1-oxo-5-isoindolinyl}(R)-2-{3-[4-(N-methyl-N-4-pyrimidinylamino)-2-chlorophenyl]-1-azetidinyl}propionamide
N-{2-[(S)-2,6-dioxo-3-piperidyl]-1-oxo-5-isoindolinyl}(R)-2-{4-[2-chloro-4-(4-methyl-2-pyridylamino)phenyl]-1-piperazinyl}butyramide
N-{2-[(S)-2,6-dioxo-3-piperidyl]-1-oxo-5-isoindolinyl}{3-[4-(N-methyl-N-2-pyrimidinylamino)-2-chlorophenyl]-1-azetidinyl}acetamide
N-{2-[(S)-2,6-dioxo-3-piperidyl]-1-oxo-5-isoindolinyl}(R)-2-{3-[4-(N-methyl-N-2-pyrimidinylamino)-2-chlorophenyl]-1-azetidinyl}propionamide
N-{2-[(S)-2,6-dioxo-3-piperidyl]-1-oxo-5-isoindolinyl}{4-[4-(1-azetidinyl)-2-(trifluoromethyl)phenyl]-1-piperazinyl}acetamide
N-{2-[(S)-2,6-dioxo-3-piperidyl]-1-oxo-5-isoindolinyl}{4-[2-(trifluoromethyl)-4-cumenyl]-1-piperazinyl}acetamide
N-{2-[(S)-2,6-dioxo-3-piperidyl]-1-oxo-5-isoindolinyl}{3-[4-(N-methyl-N-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}(R)-2-{3-[4-(N-methyl-N-2-pyrimidinylamino)-2-(trifluoromethyl)phenyl]-1-azetidinyl}propionamide
N-{2-[(S)-2,6-dioxo-3-piperidyl]-1-oxo-5-isoindolinyl}{4-[4-(4-isopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}(4-{4-[4-(1-pyrrolidinyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[2-chloro-4-(4-chloro-2-pyridylamino)phenyl]-1-piperazinyl}acetamide
N-{2-[(S)-2,6-dioxo-3-piperidyl]-1-oxo-5-isoindolinyl}{4-[4-(N-methyl-N-4-pyrimidinylamino)-2-chlorophenyl]-1-piperazinyl}acetamide
N-{2-[(S)-2,6-dioxo-3-piperidyl]-1-oxo-5-isoindolinyl}{4-[4-(5-isopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-morpholino-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[4-(N-methyl-N-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}(4-{4-[4-methyl-6-(trifluoromethyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[4-(4-cyclopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-chloro-2-(trifluoromethyl)phenyl]-1-piperazinyl}acetamide
N-{2-[(S)-2,6-dioxo-3-piperidyl]-1-oxo-5-isoindolinyl}(R)-2-{3-[4-(N-methyl-N-4-pyrimidinylamino)-2-(trifluoromethyl)phenyl]-1-azetidinyl}propionamide
N-{2-[(S)-2,6-dioxo-3-piperidyl]-1-oxo-5-isoindolinyl}(4-{4-[4-(tert-butyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[4-(N-methyl-N-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[2-chloro-4-(4-isopropyl-2-pyrimidinylamino)phenyl]-1-azetidinyl}acetamide
N-{2-[(S)-2,6-dioxo-3-piperidyl]-1-oxo-5-isoindolinyl}(3-{2-chloro-4-[4-(1-pyrrolidinyl)-2-pyrimidinylamino]phenyl}-1-azetidinyl)acetamide
N-{2-[(S)-2,6-dioxo-3-piperidyl]-1-oxo-5-isoindolinyl}{3-[2-chloro-4-(4-morpholino-2-pyrimidinylamino)phenyl]-1-azetidinyl}acetamide
N-{2-[(S)-2,6-dioxo-3-piperidyl]-1-oxo-5-isoindolinyl}(S)-2-{4-[4-(N-methyl-N-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{4-[4-(4,6-diaza-5-indanylamino)-2-(trifluoromethyl)phenyl]-1-piperazinyl}acetamide
N-{2-[(S)-2,6-dioxo-3-piperidyl]-1-oxo-5-isoindolinyl}{3-[2-chloro-4-(4-methyl-2-pyrimidinylamino)phenyl]-1-azetidinyl}acetamide
N-{2-[(S)-2,6-dioxo-3-piperidyl]-1-oxo-5-isoindolinyl}{3-[2-chloro-4-(4,6-dimethyl-2-pyrimidinylamino)phenyl]-1-azetidinyl}acetamide
N-[2-(2,6-dioxo-3-piperidyl)-1,3-dioxo-5-isoindolinyl]{3-[4-(N-methyl-N-2-pyrimidinylamino)-2-(trifluoromethyl)phenyl]-1-azetidinyl}acetamide
N-[2-(2,6-dioxo-3-piperidyl)-1,3-dioxo-5-isoindolinyl]{3-[4-(N-methyl-N-2-pyrimidinylamino)-2-chlorophenyl]-1-azetidinyl}acetamide
N-{2-[(S)-2,6-dioxo-3-piperidyl]-1-oxo-5-isoindolinyl}(S)-2-{4-[4-(N-methyl-N-4-pyrimidinylamino)-2-chlorophenyl]-1-piperazinyl}propionamide
N-{2-[(S)-2,6-dioxo-3-piperidyl]-1-oxo-5-isoindolinyl}(S)-2-{4-[4-(4,6-diaza-5-indanylamino)-2-(trifluoromethyl)phenyl]-1-piperazinyl}propionamide
N-{2-[(S)-2,6-dioxo-3-piperidyl]-1-oxo-5-isoindolinyl}(3-{4-[(6-chloro-4-pyrimidinyl)-N-methylamino]-2-(trifluoromethyl)phenyl}-1-azetidinyl)acetamide
N-{2-[(S)-2,6-dioxo-3-piperidyl]-1-oxo-5-isoindolinyl}(R)-2-(3-{4-[(6-chloro-4-pyrimidinyl)-N-methylamino]-2-(trifluoromethyl)phenyl}-1-azetidinyl)propionamide
N-{2-[(S)-2,6-dioxo-3-piperidyl]-1-oxo-5-isoindolinyl}2-{4-[4-(4,6-dimethyl-2-pyrimidinylamino)-2-(trifluoromethyl)phenyl]-1-piperazinyl}-2-methylpropionamide
N-{2-[(S)-2,6-dioxo-3-piperidyl]-1-oxo-5-isoindolinyl}(3-{4-[(4,6-dimethyl-2-pyrimidinyl)-N-methylamino]-2-(trifluoromethyl)phenyl}-1-azetidinyl)acetamide
N-{2-[(S)-2,6-dioxo-3-piperidyl]-1-oxo-5-isoindolinyl}{3-[4-chloro-2-(trifluoromethyl)phenyl]-1-azetidinyl}acetamide
N-{2-[(S)-2,6-dioxo-3-piperidyl]-1-oxo-5-isoindolinyl}2-{3-[2-chloro-4-(4,6-dimethyl-2-pyrimidinylamino)phenyl]-1-azetidinyl}-2-methylpropionamide
N-{2-[(S)-2,6-dioxo-3-piperidyl]-1-oxo-5-isoindolinyl}(4-{2-chloro-4-[(6-chloro-4-pyrimidinyl)-N-methylamino]phenyl}-1-piperazinyl)acetamide
N-{2-[(S)-2,6-dioxo-3-piperidyl]-1-oxo-5-isoindolinyl}(4-{4-[(4,6-dimethyl-2-pyrimidinyl)-N-methylamino]-2-(trifluoromethyl)phenyl}-1-piperazinyl)acetamide
N-{2-[(S)-2,6-dioxo-3-piperidyl]-1-oxo-5-isoindolinyl}(4-{4-[N-methyl(4-methyl-2-pyrimidinyl)amino]-2-(trifluoromethyl)phenyl}-1-piperazinyl)acetamide
N-{2-[(S)-2,6-dioxo-3-piperidyl]-1-oxo-5-isoindolinyl}(S)-2-(4-{4-[(4,6-dimethyl-2-pyrimidinyl)-N-methylamino]-2-(trifluoromethyl)phenyl}-1-piperazinyl)propionamide
N-{2-[(S)-2,6-dioxo-3-piperidyl]-1-oxo-5-isoindolinyl}(S)-2-(4-{4-[N-methyl(4-methyl-2-pyrimidinyl)amino]-2-(trifluoromethyl)phenyl}-1-piperazinyl)propionamide
N-{2-[(S)-2,6-dioxo-3-piperidyl]-1-oxo-5-isoindolinyl}[3-(2,4-dichlorophenyl)-1-azetidinyl]acetamide
N-{2-[(S)-2,6-dioxo-3-piperidyl]-1-oxo-5-isoindolinyl}{3-[2-chloro-4-(trifluoromethyl)phenyl]-1-azetidinyl}acetamide
N-{2-[(S)-2,6-dioxo-3-piperidyl]-1-oxo-5-isoindolinyl}(S)-2-{4-[4-(N-methyl-N-2-pyrimidinylamino)-2-(trifluoromethyl)phenyl]-1-piperazinyl}butyramide
N-{2-[(S)-2,6-dioxo-3-piperidyl]-1-oxo-5-isoindolinyl}(S)-2-(4-{4-[(4,6-dimethyl-2-pyrimidinyl)-N-methylamino]-2-(trifluoromethyl)phenyl}-1-piperazinyl)butyramide
N-{2-[(S)-2,6-dioxo-3-piperidyl]-1-oxo-5-isoindolinyl}{4-[4-(2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}1-{4-[4-(2-pyrimidinylamino)-2-(trifluoromethyl)phenyl]-1-piperazinyl}cyclopropanecarboxamide
2-[3-[4-[(6-chloropyrimidin-4-yl)amino]-2-(trifluoromethyl)phenyl]azetidin-1-yl]-N-[2-[(3S)-2,6-dioxo-3-piperidyl]-1-oxo-isoindolin-5-yl]acetamide
N-{2-[(S)-2,6-dioxo-3-piperidyl]-1-oxo-5-isoindolinyl}{3-[4-(2-methyl-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(4-methyl-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(6-methyl-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(4-chloro-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-methyl-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}1-{4-[4-(4,6-dimethyl-2-pyrimidinylamino)-2-(trifluoromethyl)phenyl]-1-piperazinyl}cyclopropanecarboxamide
N-{2-[(S)-2,6-dioxo-3-piperidyl]-1-oxo-5-isoindolinyl}(R)-2-{3-[4-(2-pyrimidinylamino)-2-(trifluoromethyl)phenyl]-1-azetidinyl}butyramide
N-{2-[(S)-2,6-dioxo-3-piperidyl]-1-oxo-5-isoindolinyl}{3-[4-(4-cyano-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(4,6-dimethyl-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-cyano-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-fluoro-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{4-[2,4-bis(trifluoromethyl)phenyl]-1-piperazinyl}acetamide
N-{2-[(S)-2,6-dioxo-3-piperidyl]-1-oxo-5-isoindolinyl}{3-[4-(6-fluoro-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-furyl)-2-(trifluoromethyl)phenyl]-1-azetidinyl}acetamide
N-{2-[(S)-2,6-dioxo-3-piperidyl]-1-oxo-5-isoindolinyl}{4-[4-(N-ethyl-N-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[4-(2-pyridyl)-2-(trifluoromethyl)phenyl]-1-azetidinyl}acetamide
N-{2-[(S)-2,6-dioxo-3-piperidyl]-1-oxo-5-isoindolinyl}(3-{4-[N-methyl(4-methyl-2-pyrimidinyl)amino]-2-(trifluoromethyl)phenyl}-1-azetidinyl)acetamide
N-{2-[(S)-2,6-dioxo-3-piperidyl]-1-oxo-5-isoindolinyl}(3-{4-[N-methyl(5-methyl-2-pyrimidinyl)amino]-2-(trifluoromethyl)phenyl}-1-azetidinyl)acetamide
N-{2-[(S)-2,6-dioxo-3-piperidyl]-1-oxo-5-isoindolinyl}{3-[4-(4,6-dimethyl-2-pyrimidinylamino)-2-(trifluoromethyl)phenyl]-1-pyrrolidinyl}acetamide
N-{2-[(S)-2,6-dioxo-3-piperidyl]-1-oxo-5-isoindolinyl}(4-{4-[N-methyl(6-methyl-4-pyrimidinyl)amino]-2-(trifluoromethyl)phenyl}-1-piperazinyl)acetamide
N-{2-[(S)-2,6-dioxo-3-piperidyl]-1-oxo-5-isoindolinyl}(4-{4-[(6-chloro-4-pyrimidinyl)-N-methylamino]-2-(trifluoromethyl)phenyl}-1-piperazinyl)acetamide
N-{2-[(S)-2,6-dioxo-3-piperidyl]-1-oxo-5-isoindolinyl}{3-[4-(2-pyrimidinylamino)-2-(trifluoromethyl)phenyl]-1-pyrrolidinyl}acetamide
N-{2-[(S)-2,6-dioxo-3-piperidyl]-1-oxo-5-isoindolinyl}(3-{4-[N-(²H₃)methyl-N-2-pyrimidinylamino]-2-(trifluoromethyl)phenyl}-1-azetidinyl)acetamide
N-{2-[(S)-2,6-dioxo-3-piperidyl]-1-oxo-5-isoindolinyl}1-{4-[4-(4-methyl-2-pyrimidinylamino)-2-(trifluoromethyl)phenyl]-1-piperazinyl}cyclopropanecarboxamide
N-{2-[(S)-2,6-dioxo-3-piperidyl]-1-oxo-5-isoindolinyl}[3-(4-bromo-2-trifluoromethoxyphenyl)-1-azetidinyl]acetamide
N-{2-[(S)-2,6-dioxo-3-piperidyl]-1-oxo-5-isoindolinyl}{3-[4-(2-pyrimidinylamino)-2-trifluoromethoxyphenyl]-1-azetidinyl}acetamide
N-{2-[(S)-2,6-dioxo-3-piperidyl]-1-oxo-5-isoindolinyl}{3-[4-(N-methyl-N-2-pyrimidinylamino)-2-trifluoromethoxyphenyl]-1-azetidinyl}acetamide
N-{2-[(S)-2,6-dioxo-3-piperidyl]-1-oxo-5-isoindolinyl}{4-[5-bromo-3-(trifluoromethyl)-2-pyridyl]-1-piperazinyl}acetamide
N-{2-[(S)-2,6-dioxo-3-piperidyl]-1-oxo-5-isoindolinyl}{4-[5-(N-methyl-N-2-pyrimidinylamino)-3-(trifluoromethyl)-2-pyridyl]-1-piperazinyl}acetamide
N-{2-[(S)-2,6-dioxo-3-piperidyl]-1-oxo-5-isoindolinyl}(4-{5-[(4-chloro-6-methyl-2-pyrimidinyl)-N-methylamino]-3-(trifluoromethyl)-2-pyridyl}-1-piperazinyl)acetamide
N-{2-[(S)-2,6-dioxo-3-piperidyl]-1-oxo-5-isoindolinyl}{4-[4-(2-pyrimidinylamino)-2-(trifluoromethyl)phenyl]-1,2,3,6-tetrahydro-1-pyridyl}acetamide
N-{2-[(S)-2,6-dioxo-3-piperidyl]-1-oxo-5-isoindolinyl}[3-(2-chloro-4-trifluoromethoxyphenyl)-1-azetidinyl]acetamide
N-{2-[(S)-2,6-dioxo-3-piperidyl]-1-oxo-5-isoindolinyl}{3-[4-fluoro-2-(trifluoromethyl)phenyl]-1-azetidinyl}acetamide
N-{2-[(S)-2,6-dioxo-3-piperidyl]-1-oxo-5-isoindolinyl}[3-(4-chloro-2-trifluoromethoxyphenyl)-1-azetidinyl]acetamide
N-{2-[(S)-2,6-dioxo-3-piperidyl]-1-oxo-5-isoindolinyl}{4-[4-(4-methyl-2-pyrimidinylamino)-2-(trifluoromethyl)phenyl]-1-piperidyl}acetamide
N-{2-[(S)-2,6-dioxo-3-piperidyl]-1-oxo-5-isoindolinyl}{6-[4-(2-pyrimidinylamino)-2-(trifluoromethyl)phenyl]-2,6-diaza-2-spiro[3.3]heptyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1-piperidyl}acetamide
N-{2-[(S)-2,6-dioxo-3-piperidyl]-1-oxo-5-isoindolinyl}{4-[3,5-bis(trifluoromethyl)-2-pyridyl]-1-piperazinyl}acetamide
N-{2-[(S)-2,6-dioxo-3-piperidyl]-1-oxo-5-isoindolinyl}1-(4-{4-[(4,6-dimethyl-2-pyrimidinyl)-N-(²H₃)methylamino]-2-(trifluoromethyl)phenyl}-1-piperazinyl)cyclopropanecarboxamide
N-{2-[(S)-2,6-dioxo-3-piperidyl]-1-oxo-5-isoindolinyl}[4-(2-chloro-4-trifluoromethoxyphenyl)-1-piperazinyl]acetamide
N-{2-[(S)-2,6-dioxo-3-piperidyl]-1-oxo-5-isoindolinyl}{3-[4-bromo-2-(trifluoromethyl)phenyl]-1-azetidinyl}acetamide
N-[2-(2,6-dioxo-3-piperidyl)-6-fluoro-1-oxo-5-isoindolinyl]{4-[4-(N-methyl-N-2-pyrimidinylamino)-2-(trifluoromethyl)phenyl]-1-piperazinyl}acetamide
N-[2-(2,6-dioxo-3-piperidyl)-6-fluoro-1-oxo-5-isoindolinyl]{4-[2,4-bis(trifluoromethyl)phenyl]-1-piperazinyl}acetamide
N-[2-(2,6-dioxo-3-piperidyl)-6-fluoro-1-oxo-5-isoindolinyl]{3-[2-chloro-4-(trifluoromethyl)phenyl]-1-azetidinyl}acetamide
N-{2-[(S)-2,6-dioxo-3-piperidyl]-1-oxo-5-isoindolinyl}(4-{5-[N-(²H₃)methyl-N-2-pyrimidinylamino]-3-(trifluoromethyl)-2-pyridyl}-1-piperazinyl)acetamide
N-{2-[(S)-2,6-dioxo-3-piperidyl]-1-oxo-5-isoindolinyl}{4-[4-(2-pyridyloxy)-2-(trifluoromethyl)phenyl]-1-piperazinyl}acetamide
N-{2-[(S)-2,6-dioxo-3-piperidyl]-1-oxo-5-isoindolinyl}{4-[4-(4,6-dimethyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{3-methyl-3-[4-(2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{4-[2-chloro-4-(4,6-dimethyl-2-pyrimidinyloxy)phenyl]-1-piperazinyl}acetamide
N-{2-[(S)-2,6-dioxo-3-piperidyl]-1-oxo-5-isoindolinyl}{4-[4-(4-methyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(4-methyl-2-pyrimidinyloxy)phenyl]-1-piperazinyl}acetamide
N-{2-[(S)-2,6-dioxo-3-piperidyl]-1-oxo-5-isoindolinyl}{4-[2-chloro-4-(6-methyl-4-pyrimidinyloxy)phenyl]-1-piperazinyl}acetamide
N-{2-[(S)-2,6-dioxo-3-piperidyl]-1-oxo-5-isoindolinyl}{4-[4-(6-methyl-4-pyrimidinyloxy)-2-(trifluoromethyl)phenyl]-1-piperazinyl}acetamide
N-{2-[(S)-2,6-dioxo-3-piperidyl]-6-fluoro-1-oxo-5-isoindolinyl}{4-[2,4-bis(trifluoromethyl)phenyl]-1-piperazinyl}acetamide
N-{2-[(S)-2,6-dioxo-3-piperidyl]-6-fluoro-1-oxo-5-isoindolinyl}{4-[2-chloro-4-(2-pyridylamino)phenyl]-1-piperazinyl}acetamide
N-{2-[(S)-2,6-dioxo-3-piperidyl]-6-fluoro-1-oxo-5-isoindolinyl}{4-[2-chloro-4-(4-methyl-2-pyrimidinyloxy)phenyl]-1-piperazinyl}acetamide
N-{2-[(S)-2,6-dioxo-3-piperidyl]-6-fluoro-1-oxo-5-isoindolinyl}{4-[2-chloro-4-(6-methyl-4-pyrimidinyloxy)phenyl]-1-piperazinyl}acetamide
N-{2-[(S)-2,6-dioxo-3-piperidyl]-6-fluoro-1-oxo-5-isoindolinyl}{4-[4-(4-methyl-2-pyrimidinyloxy)-2-(trifluoromethyl)phenyl]-1-piperazinyl}acetamide
N-[2-(2,6-dioxo-3-piperidyl)-4-fluoro-1-oxo-5-isoindolinyl]{4-[2-chloro-4-(2-pyridylamino)phenyl]-1-piperazinyl}acetamide
N-[2-(2,6-dioxo-3-piperidyl)-4-fluoro-1-oxo-5-isoindolinyl]{4-[2,4-bis(trifluoromethyl)phenyl]-1-piperazinyl}acetamide
N-[2-(2,6-dioxo-3-piperidyl)-4-fluoro-1-oxo-5-isoindolinyl]{4-[2-chloro-4-(6-methyl-4-pyrimidinyloxy)phenyl]-1-piperazinyl}acetamide
N-[2-(2,6-dioxo-3-piperidyl)-4-fluoro-1-oxo-5-isoindolinyl]{4-[2-chloro-4-(4-methyl-2-pyrimidinyloxy)phenyl]-1-piperazinyl}acetamide
N-[2-(2,6-dioxo-3-piperidyl)-4-fluoro-1-oxo-5-isoindolinyl]{4-[4-(4-methyl-2-pyrimidinyloxy)-2-(trifluoromethyl)phenyl]-1-piperazinyl}acetamide
N-{2-[(S)-2,6-dioxo-3-piperidyl]-6-fluoro-1-oxo-5-isoindolinyl}{3-[4-(2-pyrimidinyloxy)-2-(trifluoromethyl)phenyl]-1-azetidinyl}acetamide
N-{2-[(S)-2,6-dioxo-3-piperidyl]-6-fluoro-1-oxo-5-isoindolinyl}{3-[4-(6-methyl-4-pyrimidinyloxy)-2-(trifluoromethyl)phenyl]-1-azetidinyl}acetamide
N-{2-[(S)-2,6-dioxo-3-piperidyl]-6-fluoro-1-oxo-5-isoindolinyl}{3-[2-chloro-4-(6-methyl-4-pyrimidinyloxy)phenyl]-1-azetidinyl}acetamide
N-{2-[(S)-2,6-dioxo-3-piperidyl]-6-fluoro-1-oxo-5-isoindolinyl}{3-[2-chloro-4-(2-pyrimidinyloxy)phenyl]-1-azetidinyl}acetamide
N-{2-[(S)-2,6-dioxo-3-piperidyl]-1-oxo-5-isoindolinyl}{3-[2-chloro-4-(methylamino)phenyl]-1-azetidinyl}acetamide
N-{2-[(S)-2,6-dioxo-3-piperidyl]-1-oxo-5-isoindolinyl}{3-[4-(methylamino)-2-(trifluoromethyl)phenyl]-1-azetidinyl}acetamide
N-{2-[(R)-2,6-dioxo-3-piperidyl]-4-fluoro-1-oxo-5-isoindolinyl}{3-[2-chloro-4-(6-methyl-4-pyrimidinyloxy)phenyl]-1-azetidinyl}acetamide
N-{2-[(S)-2,6-dioxo-3-piperidyl]-4-fluoro-1-oxo-5-isoindolinyl}{3-[2-chloro-4-(2-pyrimidinyloxy)phenyl]-1-azetidinyl}acetamide
N-[2-(2,6-dioxo-3-piperidyl)-3-oxo-2,5-diaza-6-indanyl]{4-[2-chloro-4-(2-pyridylamino)phenyl]-1-piperazinyl}acetamide
N-[2-(2,6-dioxo-3-piperidyl)-1-oxo-2,4-diaza-5-indanyl]{4-[2-chloro-4-(2-pyridylamino)phenyl]-1-piperazinyl}acetamide
3-{5-[2-(4-{4-[5-(benzyloxy)-2-pyridylamino]-2-(trifluoromethyl)phenyl}-1-piperazinyl)ethoxy]-1-oxo-2-isoindolinyl}-2,6-piperidinedione
3-[5-(2-{4-[2,4-bis(trifluoromethyl)phenyl]-1-piperidyl}ethoxy)-1-oxo-2-isoindolinyl]-2,6-piperidinedione
3-[5-(2-{3-[2,4-bis(trifluoromethyl)phenyl]-1-azetidinyl}ethoxy)-1-oxo-2-isoindolinyl]-2,6-piperidinedione
N-[2-(2,6-dioxo-3-piperidyl)-1-oxo-2,4-diaza-5-indanyl]{4-[4-(4-methyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{4-[4-bromo-2-(hydroxymethyl)phenyl]-1-piperazinyl}acetamide
3-(5-{2-[4-(4-{[2-(benzyloxy)ethyl]-N-2-pyridylamino}-2-chlorophenyl)-1-piperazinyl]ethoxy}-1-oxo-2-isoindolinyl)-2,6-piperidinedione
3-{5-[2-(4-{4-[4-(benzyloxy)-2-pyridylamino]-2-chlorophenyl}-1-piperazinyl)ethoxy]-1-oxo-2-isoindolinyl}-2,6-piperidinedione
N-{2-[(S)-2,6-dioxo-3-piperidyl]-1-oxo-5-isoindolinyl}[4-(4-{[5-(hydroxymethyl)-2-pyrimidinyl]-N-methylamino}-2-(trifluoromethyl)phenyl)-1-piperazinyl]acetamide
N-{2-[(S)-2,6-dioxo-3-piperidyl]-1-oxo-5-isoindolinyl}[4-(4-{[4-(hydroxymethyl)-2-pyrimidinyl]-N-methylamino}-2-(trifluoromethyl)phenyl)-1-piperazinyl]acetamide
3-[5-(2-{4-[2-chloro-4-(4-hydroxy-2-pyridylamino)phenyl]-1-piperazinyl}ethoxy)-1-oxo-2-isoindolinyl]-2,6-piperidinedione
3-{5-[2-(4-{2-chloro-4-[(2-hydroxyethyl)-N-2-pyridylamino]phenyl}-1-piperazinyl)ethoxy]-1-oxo-2-isoindolinyl}-2,6-piperidinedione
N-{2-[(S)-2,6-dioxo-3-piperidyl]-1-oxo-5-isoindolinyl}{3-[4-hydroxy-2-(trifluoromethyl)phenyl]-1-azetidinyl}acetamide
N-{2-[(S)-2,6-dioxo-3-piperidyl]-1-oxo-5-isoindolinyl}(4-{4-[2-(benzyloxy)ethoxy]-2-chlorophenyl}-1-piperazinyl)acetamide
N-{2-[(S)-2,6-dioxo-3-piperidyl]-1-oxo-5-isoindolinyl}{4-[2-chloro-4-(2-hydroxyethoxy)phenyl]-1-piperazinyl}acetamide
N-{2-[(S)-2,6-dioxo-3-piperidyl]-1-oxo-5-isoindolinyl}{4-[2-chloro-4-(1-cyanocyclopropyl)phenyl]-1-piperazinyl}acetamide
N-methyl-N-{4-[1-({N-2-[(S)-2,6-dioxo-3-piperidyl]-1-oxo-5-isoindolinylcarbamoyl}methyl)-3-azetidinyl]-3-(trifluoromethyl)phenyl}glycolamide
N-{4-[4-({N-2-[(S)-2,6-dioxo-3-piperidyl]-1-oxo-5-isoindolinylcarbamoyl}methyl)-1-piperazinyl]-3-(trifluoromethyl)phenyl}glycolamide
N-{2-[(S)-2,6-dioxo-3-piperidyl]-1-oxo-5-isoindolinyl}{4-[2-chloro-4-(hydroxymethyl)phenyl]-1-piperazinyl}acetamide
N-{2-[(S)-2,6-dioxo-3-piperidyl]-1-oxo-5-isoindolinyl}{4-[4-(N-methyl{5-[(methylamino)methyl]-2-pyrimidinyl}amino)-2-(trifluoromethyl)phenyl]-1-piperazinyl}acetamide
3-[5-(2-{4-[2-chloro-4-(5-{2-[2-(methylamino)ethoxy]ethyl}-2-pyridylamino)phenyl]-1-piperazinyl}ethoxy)-1-oxo-2-isoindolinyl]-2,6-piperidinedione
3-[5-(2-{4-[2-chloro-4-(5-{2-[2-(methylamino)ethoxy]ethyl}-2-pyridylamino)phenyl]-1-piperazinyl}ethoxy)-1-oxo-2-isoindolinyl]-2,6-piperidinedione
3-(5-{2-[4-(2-chloro-4-{5-[2-(methylamino)ethyl]-2-pyridylamino}phenyl)-1-piperazinyl]ethoxy}-1-oxo-2-isoindolinyl)-2,6-piperidinedione
N-{2-[(S)-2,6-dioxo-3-piperidyl]-1-oxo-5-isoindolinyl}{4-[2-bromo-4-(N-methyl-N-2-pyrimidinylamino)phenyl]-1-piperazinyl}acetamide
3-{[3-chloro-4-(4-{2-[2-(2,6-dioxo-3-piperidyl)-1-oxo-5-isoindolinyloxy]ethyl}-1-piperazinyl)phenyl]-N-2-pyridylamino}propiononitrile
N-{2-[(S)-2,6-dioxo-3-piperidyl]-1-oxo-5-isoindolinyl}(4-{4-[1-(aminomethyl)cyclopropyl]-2-chlorophenyl}-1-piperazinyl)acetamide
3-{5-[2-(4-{4-[(3-aminopropyl)-N-2-pyridylamino]-2-chlorophenyl}-1-piperazinyl)ethoxy]-1-oxo-2-isoindolinyl}-2,6-piperidinedione
N-{2-[(R)-2,6-dioxo-3-piperidyl]-1-oxo-5-isoindolinyl}{3-[2-chloro-4-(p-hydroxyphenylamino)phenyl]-1-azetidinyl}acetamide
N-{2-[(R)-2,6-dioxo-3-piperidyl]-1-oxo-5-isoindolinyl}{3-[2-chloro-4-(o-hydroxyphenylamino)phenyl]-1-azetidinyl}acetamide
N-{2-[(S)-2,6-dioxo-3-piperidyl]-1-oxo-5-isoindolinyl}{4-[2-chloro-4-(1-piperazinyl)phenyl]-1-piperazinyl}acetamide
N-{2-[(S)-2,6-dioxo-3-piperidyl]-1-oxo-5-isoindolinyl}{3-[2-chloro-4-(5-hydroxy-2-pyrimidinylamino)phenyl]-1-azetidinyl}acetamide
N-{2-[(S)-2,6-dioxo-3-piperidyl]-1-oxo-5-isoindolinyl}{3-[2-chloro-4-(5-hydroxy-2-pyridylamino)phenyl]-1-azetidinyl}acetamide
N-{2-[(S)-2,6-dioxo-3-piperidyl]-1-oxo-5-isoindolinyl}{3-[4-(5-formyl-2-pyridylamino)-2-(trifluoromethyl)phenyl]-1-azetidinyl}acetamide
6-[4-(1-{2-[2-(2,6-dioxo-3-piperidyl)-1-oxo-5-isoindolinyloxy]ethyl}-3-azetidinyl)-3-(trifluoromethyl)phenylamino]nicotinaldehyde
N-{2-[(S)-2,6-dioxo-3-piperidyl]-1-oxo-5-isoindolinyl}{3-[2-chloro-4-(m-hydroxyphenylamino)phenyl]-1-azetidinyl}acetamide
3-[5-(2-{4-[2-chloro-4-(4-{2-[2-(methylamino)ethoxy]ethyl}-2-pyridylamino)phenyl]-1-piperazinyl}ethoxy)-1-oxo-2-isoindolinyl]-2,6-piperidinedione
3-(5-{2-[4-(2-chloro-4-{[2-(methylamino)ethyl]-N-2-pyridylamino}phenyl)-1-piperazinyl]ethoxy}-1-oxo-2-isoindolinyl)-2,6-piperidinedione
3-[5-(2-{3-[2-chloro-4-(m-hydroxyphenylamino)phenyl]-1-azetidinyl}ethoxy)-1-oxo-2-isoindolinyl]-2,6-piperidinedione
3-[5-(2-{3-[2-chloro-4-(m-hydroxyphenylamino)phenyl]-1-azetidinyl}ethoxy)-1-oxo-2-isoindolinyl]-2,6-piperidinedione
3-[5-(2-{3-[2-chloro-4-(m-hydroxyphenylamino)phenyl]-1-azetidinyl}ethoxy)-1-oxo-2-isoindolinyl]-2,6-piperidinedione
3-[5-(2-{3-[4-(1,6-diaza-5,6,7,8-tetrahydro-2-naphthylamino)-2-(trifluoromethyl)phenyl]-1-azetidinyl}ethoxy)-1-oxo-2-isoindolinyl]-2,6-piperidinedione
3-[5-(2-{3-[2-chloro-4-(1,6-diaza-5,6,7,8-tetrahydro-2-naphthylamino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{3-[2-chloro-4-(1,6-diaza-5,6,7,8-tetrahydro-2-naphthylamino)phenyl]-1-azetidinyl}acetamide
N-[2-(2,6-dioxo-3-piperidyl)-1-oxo-5-isoindolinyl][3-(4-{5-[(methylamino)methyl]-2-pyridylamino}-2-(trifluoromethyl)phenyl)-1-azetidinyl]acetamide
N-{2-[(S)-2,6-dioxo-3-piperidyl]-1-oxo-5-isoindolinyl}{4-[2-chloro-4-(4-piperidylamino)phenyl]-1-piperazinyl}acetamide
N-{2-[(R)-2,6-dioxo-3-piperidyl]-1-oxo-5-isoindolinyl}{3-[4-(1,6-diaza-5,6,7,8-tetrahydro-2-naphthylamino)-2-(trifluoromethyl)phenyl]-1-azetidinyl}acetamide
3-(5-{2-[3-(4-{5-[(methylamino)methyl]-2-pyridylamino}-2-(trifluoromethyl)phenyl)-1-azetidinyl]ethoxy}-1-oxo-2-isoindolinyl)-2,6-piperidinedione
3-(5-{2-[3-(4-{5-[(methylamino)methyl]-2-pyrimidinylamino}-2-(trifluoromethyl)phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}[3-(2-chloro-4-{5-[(methylamino)methyl]-2-pyrimidinylamino}phenyl)-1-azetidinyl]acetamide
N-{2-[(R)-2,6-dioxo-3-piperidyl]-1-oxo-5-isoindolinyl}[3-(2-chloro-4-{m-[2-(methylamino)ethoxy]phenylamino}phenyl)-1-azetidinyl]acetamide
3-(5-{2-[3-(2-chloro-4-{5-[(methylamino)methyl]-2-pyrimidinylamino}phenyl)-1-azetidinyl]ethoxy}-1-oxo-2-isoindolinyl)-2,6-piperidinedione
N-{2-[(R)-2,6-dioxo-3-piperidyl]-1-oxo-5-isoindolinyl}[3-(4-{5-[(methylamino)methyl]-2-pyrimidinylamino}-2-(trifluoromethyl)phenyl)-1-azetidinyl]acetamide
3-(5-{2-[3-(2-chloro-4-{m-[(methylamino)methyl]phenylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}[3-(2-chloro-4-{p-[(methylamino)methyl]phenylamino}phenyl)-1-azetidinyl]acetamide
N-{2-[(S)-2,6-dioxo-3-piperidyl]-1-oxo-5-isoindolinyl}[3-(2-chloro-4-{m-[(methylamino)methyl]phenylamino}phenyl)-1-azetidinyl]acetamide
N-{2-[(S)-2,6-dioxo-3-piperidyl]-1-oxo-5-isoindolinyl}(4-{2-chloro-4-[5-(hydroxymethyl)-2-pyridylamino]phenyl}-1-piperazinyl)acetamide
3-(5-{2-[3-(2-chloro-4-{p-[(methylamino)methyl]phenylamino}phenyl)-1-azetidinyl]ethoxy}-1-oxo-2-isoindolinyl)-2,6-piperidinedione
3-{5-[2-(4-{2-chloro-4-[5-(hydroxymethyl)-2-pyridylamino]phenyl}-1-piperazinyl)ethoxy]-1-oxo-2-isoindolinyl}-2,6-piperidinedione 2-[(S)-2,6-dioxo-3-piperidyl]-6-fluoro-1-oxo-5-isoindolinyl (4-{2-chloro-4-[5-(hydroxymethyl)-2-pyridylamino]phenyl}-1-piperazinyl)acetate
N-{2-[(S)-2,6-dioxo-3-piperidyl]-1-oxo-5-isoindolinyl}(4-{2-chloro-4-[4-(hydroxymethyl)-2-pyridylamino]phenyl}-1-piperazinyl)acetamide
3-{5-[2-(4-{2-chloro-4-[4-(hydroxymethyl)-2-pyridylamino]phenyl}-1-piperazinyl)ethoxy]-1-oxo-2-isoindolinyl}-2,6-piperidinedione
2-[(S)-2,6-dioxo-3-piperidyl]-6-fluoro-1-oxo-5-isoindolinyl (4-{2-chloro-4-[4-(hydroxymethyl)-2-pyridylamino]phenyl}-1-piperazinyl)acetate
N-{2-[(S)-2,6-dioxo-3-piperidyl]-1-oxo-5-isoindolinyl}(4-{2-chloro-4-[6-(hydroxymethyl)-2-pyridylamino]phenyl}-1-piperazinyl)acetamide
3-{5-[2-(4-{2-chloro-4-[6-(hydroxymethyl)-2-pyridylamino]phenyl}-1-piperazinyl)ethoxy]-1-oxo-2-isoindolinyl}-2,6-piperidinedione
N-{2-[(S)-2,6-dioxo-3-piperidyl]-6-fluoro-1-oxo-5-isoindolinyl}(4-{2-chloro-4-[6-(hydroxymethyl)-2-pyridylamino]phenyl}-1-piperazinyl)acetamide
N-{2-[(S)-2,6-dioxo-3-piperidyl]-1-oxo-5-isoindolinyl}{3-[4-(2,4,6-triaza-5-indanylamino)-2-(trifluoromethyl)phenyl]-1-azetidinyl}acetamide
N-{2-[(S)-2,6-dioxo-3-piperidyl]-1-oxo-5-isoindolinyl}{3-[4-(2,4,6-triaza-5-indanylamino)-2-(trifluoromethyl)phenyl]-1-azetidinyl}acetamide
N-{2-[(S)-2,6-dioxo-3-piperidyl]-1-oxo-5-isoindolinyl}{3-[4-(3-hydroxy-2-pyridylamino)-2-(trifluoromethyl)phenyl]-1-azetidinyl}acetamide
N-{2-[(R)-2,6-dioxo-3-piperidyl]-1-oxo-5-isoindolinyl}{3-[4-(o-hydroxyphenylamino)-2-(trifluoromethyl)phenyl]-1-azetidinyl}acetamide
N-{2-[(S)-2,6-dioxo-3-piperidyl]-1-oxo-5-isoindolinyl}[3-(2-methoxy-4-{4-[(methylamino)methyl]-2-pyrimidinylamino}phenyl)-1-azetidinyl]acetamide
N-{2-[(S)-2,6-dioxo-3-piperidyl]-1-oxo-5-isoindolinyl}[4-(2-chloro-4-{6-[(methylamino)methyl]-2-pyridylamino}phenyl)-1-piperazinyl]acetamide
3-(5-{2-[4-(2-chloro-4-{6-[(methylamino)methyl]-2-pyridylamino}phenyl)-1-piperazinyl]ethoxy}-1-oxo-2-isoindolinyl)-2,6-piperidinedione
N-{2-[(S)-2,6-dioxo-3-piperidyl]-6-fluoro-1-oxo-5-isoindolinyl}[4-(2-chloro-4-{6-[(methylamino)methyl]-2-pyridylamino}phenyl)-1-piperazinyl]acetamide
N-[2-(2-{2-[3-chloro-4-(4-{2-[2-(2,6-dioxo-3-piperidyl)-1-oxo-5-isoindolinyloxy]ethyl}-1-piperazinyl)phenylamino]-4-pyridyl}ethoxy)ethyl]-N-methylglycolamide
N-[2-(m-{3-chloro-4-[1-({N-2-[(R)-2,6-dioxo-3-piperidyl]-1-oxo-5-isoindolinylcarbamoyl}methyl)-3-azetidinyl]phenylamino}phenoxy)ethyl]-N-methylglycolamide
N-(2-{[3-chloro-4-(4-{2-[2-(2,6-dioxo-3-piperidyl)-1-oxo-5-isoindolinyloxy]ethyl}-1-piperazinyl)phenyl]-N-2-pyridylamino}ethyl)-N-methylglycolamide
N-({2-[3-chloro-4-(1-{2-[2-(2,6-dioxo-3-piperidyl)-1-oxo-5-isoindolinyloxy]ethyl}-3-azetidinyl)phenylamino]-5-pyrimidinyl}methyl)-N-methylglycolamide
N-{2-[(S)-2,6-dioxo-3-piperidyl]-1-oxo-5-isoindolinyl}[4-(2-chloro-4-{5-[2-(methylamino)ethoxy]-2-pyridylamino}phenyl)-1-piperazinyl]acetamide
N-{2-[(S)-2,6-dioxo-3-piperidyl]-1-oxo-5-isoindolinyl}[4-(2-chloro-4-{5-[2-(methylamino)ethoxy]-2-pyridylamino}phenyl)-1-piperazinyl]acetamide
3-(5-{2-[4-(2-chloro-4-{5-[2-(methylamino)ethoxy]-2-pyridylamino}phenyl)-1-piperazinyl]ethoxy}-1-oxo-2-isoindolinyl)-2,6-piperidinedione
N-{2-[(S)-2,6-dioxo-3-piperidyl]-6-fluoro-1-oxo-5-isoindolinyl}[4-(2-chloro-4-{5-[2-(methylamino)ethoxy]-2-pyridylamino}phenyl)-1-piperazinyl]acetamide
N-{2-[(R)-2,6-dioxo-3-piperidyl]-1-oxo-5-isoindolinyl}{4-[4-(N-methyl{4-[(methylamino)methyl]-2-pyrimidinyl}amino)-2-(trifluoromethyl)phenyl]-1-piperazinyl}acetamide
N-methyl-N-({6-[4-(1-{[N-2-(2,6-dioxo-3-piperidyl)-1-oxo-5-isoindolinylcarbamoyl]methyl}-3-azetidinyl)-3-(trifluoromethyl)phenylamino]-3-pyridyl}methyl)glycolamide
N-{2-[(R)-2,6-dioxo-3-piperidyl]-1-oxo-5-isoindolinyl}{3-[4-(6-glycoloyl-1,6-diaza-5,6,7,8-tetrahydro-2-naphthylamino)-2-(trifluoromethyl)phenyl]-1-azetidinyl}acetamide
3-(5-{2-[3-(4-{m-[2-(methylamino)ethyl]phenylamino}-2-(trifluoromethyl)phenyl)-1-azetidinyl]ethoxy}-1-oxo-2-isoindolinyl)-2,6-piperidinedione
3-[5-(2-{3-[4-(6-glycoloyl-1,6-diaza-5,6,7,8-tetrahydro-2-naphthylamino)-2-(trifluoromethyl)phenyl]-1-azetidinyl}ethoxy)-1-oxo-2-isoindolinyl]-2,6-piperidinedione
N-[2-(2-{6-[3-chloro-4-(4-{2-[2-(2,6-dioxo-3-piperidyl)-1-oxo-5-isoindolinyloxy]ethyl}-1-piperazinyl)phenylamino]-3-pyridyl}ethoxy)ethyl]-N-methylglycolamide
N-{2-[(S)-2,6-dioxo-3-piperidyl]-1-oxo-5-isoindolinyl}[3-(4-{m-[2-(methylamino)ethyl]phenylamino}-2-(trifluoromethyl)phenyl)-1-azetidinyl]acetamide
N-{2-[(S)-2,6-dioxo-3-piperidyl]-1-oxo-5-isoindolinyl}[4-(2-chloro-4-{4-[2-(methylamino)ethoxy]-2-pyridylamino}phenyl)-1-piperazinyl]acetamide
3-(5-{2-[4-(2-chloro-4-{4-[2-(methylamino)ethoxy]-2-pyridylamino}phenyl)-1-piperazinyl]ethoxy}-1-oxo-2-isoindolinyl)-2,6-piperidinedione
N-{2-[(S)-2,6-dioxo-3-piperidyl]-6-fluoro-1-oxo-5-isoindolinyl}[4-(2-chloro-4-{4-[2-(methylamino)ethoxy]-2-pyridylamino}phenyl)-1-piperazinyl]acetamide
N-{2-[(S)-2,6-dioxo-3-piperidyl]-1-oxo-5-isoindolinyl}[3-(2-chloro-4-{m-[2-(methylamino)ethyl]phenylamino}phenyl)-1-azetidinyl]acetamide
N-{2-[(S)-2,6-dioxo-3-piperidyl]-1-oxo-5-isoindolinyl}[3-(2-chloro-4-{m-[2-(methylamino)ethyl]phenylamino}phenyl)-1-azetidinyl]acetamide
N-(2-{6-[3-chloro-4-(4-{2-[2-(2,6-dioxo-3-piperidyl)-1-oxo-5-isoindolinyloxy]ethyl}-1-piperazinyl)phenylamino]-3-pyridyl}ethyl)-N-methylglycolamide
N-{2-[(S)-2,6-dioxo-3-piperidyl]-1-oxo-5-isoindolinyl}{4-[2-chloro-4-(4-glycoloyl-1-piperazinyl)phenyl]-1-piperazinyl}acetamide
N-[(6-{3-chloro-4-[4-({N-2-[(S)-2,6-dioxo-3-piperidyl]-1-oxo-5-isoindolinylcarbamoyl}methyl)-1-piperazinyl]phenylamino}-2-pyridyl)methyl]-N-methylglycolamide
3-(5-{2-[4-(2-chloro-4-{4-[2-(methylamino)ethyl]-2-pyridylamino}phenyl)-1-piperazinyl]ethoxy}-1-oxo-2-isoindolinyl)-2,6-piperidinedione
N-{2-[(S)-2,6-dioxo-3-piperidyl]-1-oxo-5-isoindolinyl}[3-(2-chloro-4-{o-[2-(methylamino)ethyl]phenylamino}phenyl)-1-azetidinyl]acetamide
N-{2-[(S)-2,6-dioxo-3-piperidyl]-1-oxo-5-isoindolinyl}{3-[2-chloro-4-(4-isoindolinylamino)phenyl]-1-azetidinyl}acetamide
N-methyl-N-{[2-(N-methyl{4-[4-({N-2-[(R)-2,6-dioxo-3-piperidyl]-1-oxo-5-isoindolinylcarbamoyl}methyl)-1-piperazinyl]-3-(trifluoromethyl)phenyl}amino)-5-pyrimidinyl]methyl}glycolamide
N-methyl-N-{[2-(N-methyl{4-[4-({N-2-[(S)-2,6-dioxo-3-piperidyl]-1-oxo-5-isoindolinylcarbamoyl}methyl)-1-piperazinyl]-3-(trifluoromethyl)phenyl}amino)-5-pyrimidinyl]methyl}(methylamino)acetamide
N-methyl-N-{[2-(N-methyl{4-[4-({N-2-[(R)-2,6-dioxo-3-piperidyl]-1-oxo-5-isoindolinylcarbamoyl}methyl)-1-piperazinyl]-3-(trifluoromethyl)phenyl}amino)-5-pyrimidinyl]methyl}(R)-2-aminopropionamide
N-{2-[(S)-2,6-dioxo-3-piperidyl]-1-oxo-5-isoindolinyl}{3-[2-chloro-4-(5-isoindolinylamino)phenyl]-1-azetidinyl}acetamide
N-methyl-N-{[2-(N-methyl{4-[4-({N-2-[(R)-2,6-dioxo-3-piperidyl]-1-oxo-5-isoindolinylcarbamoyl}methyl)-1-piperazinyl]-3-(trifluoromethyl)phenyl}amino)-5-pyrimidinyl]methyl}aminoacetamide
N-{2-[(S)-2,6-dioxo-3-piperidyl]-1-oxo-5-isoindolinyl}[3-(2-chloro-4-{o-[(methylamino)methyl]phenylamino}phenyl)-1-azetidinyl]acetamide
N-[2-(6-{3-chloro-4-[4-({N-2-[(S)-2,6-dioxo-3-piperidyl]-1-oxo-5-isoindolinylcarbamoyl}methyl)-1-piperazinyl]phenylamino}-3-pyridyloxy)ethyl]-N-methylglycolamide
N-{2-[(S)-2,6-dioxo-3-piperidyl]-1-oxo-5-isoindolinyl}{4-[2-chloro-4-(N-methyl{m-[2-(methylamino)ethoxy]phenyl}amino)phenyl]-1-piperazinyl}acetamide
N-{2-[(S)-2,6-dioxo-3-piperidyl]-1-oxo-5-isoindolinyl}{4-[2-chloro-4-(N-methyl{m-[2-(methylamino)ethoxy]phenyl}amino)phenyl]-1-piperazinyl}acetamide
N-{2-[(S)-2,6-dioxo-3-piperidyl]-1-oxo-5-isoindolinyl}{4-[2-chloro-4-(N-methyl{m-[2-(methylamino)ethoxy]phenyl}amino)phenyl]-1-piperazinyl}acetamide
N-{2-[(S)-2,6-dioxo-3-piperidyl]-1-oxo-5-isoindolinyl}{4-[2-chloro-4-(N-methyl{m-[2-(methylamino)ethoxy]phenyl}amino)phenyl]-1-piperazinyl}acetamide
3-[5-(2-{4-[2-chloro-4-(N-methyl{m-[2-(methylamino)ethoxy]phenyl}amino)phenyl]-1-piperazinyl}ethoxy)-1-oxo-2-isoindolinyl]-2,6-piperidinedione
N-{2-[(S)-2,6-dioxo-3-piperidyl]-1-oxo-5-isoindolinyl}[3-(3-chloro-4′-hydroxy-4-biphenylyl)-1-azetidinyl]acetamide
N-{2-[(S)-2,6-dioxo-3-piperidyl]-1-oxo-5-isoindolinyl}[3-(4-{p-[2-(methylamino)ethoxy]phenylamino}-2-(trifluoromethyl)phenyl)-1-azetidinyl]acetamide
N-{2-[(S)-2,6-dioxo-3-piperidyl]-1-oxo-5-isoindolinyl}{3-[4-(2,4-diaza-5-indanylamino)-2-(trifluoromethyl)phenyl]-1-azetidinyl}acetamide
N-{2-[(S)-2,6-dioxo-3-piperidyl]-1-oxo-5-isoindolinyl}{3-[4-(2,4-diaza-6-indanylamino)-2-(trifluoromethyl)phenyl]-1-azetidinyl}acetamide
N-{2-[(R)-2,6-dioxo-3-piperidyl]-1-oxo-5-isoindolinyl}[3-(4-{o-[2-(methylamino)ethoxy]phenylamino}-2-(trifluoromethyl)phenyl)-1-azetidinyl]acetamide
N-{2-[(S)-2,6-dioxo-3-piperidyl]-1-oxo-5-isoindolinyl}[3-(4-{m-[2-(methylamino)ethoxy]phenylamino}-2-(trifluoromethyl)phenyl)-1-azetidinyl]acetamide
N-{2-[m-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethyl}-N-methylglycolamide
N-{2-[m-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethyl}-N-methylglycolamide
N-{2-[m-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethyl}-N-methylglycolamide
N-{2-[(S)-2,6-dioxo-3-piperidyl]-1-oxo-5-isoindolinyl}{4-[4-(cyclohexylamino)-2-(trifluoromethyl)phenyl]-1-piperazinyl}acetamide
3-[1-oxo-5-(2-{3-[4-(2,4,6-triaza-5-indanylamino)-2-(trifluoromethyl)phenyl]-1-azetidinyl}ethoxy)-2-isoindolinyl]-2,6-piperidinedione
N-{2-[(S)-2,6-dioxo-3-piperidyl]-1-oxo-5-isoindolinyl}{3-[4-(2-glycoloyl-2,4,6-triaza-5-indanylamino)-2-(trifluoromethyl)phenyl]-1-azetidinyl}acetamide
N-methyl-N-[2-(p-{4-[1-({N-2-[(S)-2,6-dioxo-3-piperidyl]-1-oxo-5-isoindolinylcarbamoyl}methyl)-3-azetidinyl]-3-(trifluoromethyl)phenylamino}phenoxy)ethyl]glycolamide
6-{4-[1-({N-2-[(S)-2,6-dioxo-3-piperidyl]-1-oxo-5-isoindolinylcarbamoyl}methyl)-3-azetidinyl]-3-(trifluoromethyl)phenylamino}-2-pyridinecarboxylic acid
N-{2-[(S)-2,6-dioxo-3-piperidyl]-1-oxo-5-isoindolinyl}{3-[2-chloro-4-(2-glycoloyl-5-isoindolinylamino)phenyl]-1-azetidinyl}acetamide
N-{2-[(R)-2,6-dioxo-3-piperidyl]-1-oxo-5-isoindolinyl}{3-[4-(p-{2-[2-(methylamino)ethoxy]ethyl}phenylamino)-2-(trifluoromethyl)phenyl]-1-azetidinyl}acetamide
tert-butyl 6-{4-[1-({N-2-[(S)-2,6-dioxo-3-piperidyl]-1-oxo-5-isoindolinylcarbamoyl}methyl)-3-azetidinyl]-3-(trifluoromethyl)phenylamino}-2-pyridinecarboxylate
3-[5-(2-{3-[2-chloro-4-(5-isoindolinylamino)phenyl]-1-azetidinyl}ethoxy)-1-oxo-2-isoindolinyl]-2,6-piperidinedione
3-[5-(2-{4-[2-chloro-4-(2,4,6-triaza-5-indanylamino)phenyl]-1-piperazinyl}ethoxy)-1-oxo-2-isoindolinyl]-2,6-piperidinedione
N-{2-[(S)-2,6-dioxo-3-piperidyl]-1-oxo-5-isoindolinyl}{4-[2-chloro-4-(2,4,6-triaza-5-indanylamino)phenyl]-1-piperazinyl}acetamide
3-(5-{2-[3-(4-{p-[2-(methylamino)ethoxy]phenylamino}-2-(trifluoromethyl)phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}{4-[2-(hydroxymethyl)-4-(N-methyl-N-2-pyrimidinylamino)phenyl]-1-piperazinyl}acetamide
N-{2-[(S)-2,6-dioxo-3-piperidyl]-1-oxo-5-isoindolinyl}{4-[2-(hydroxymethyl)-4-(N-methyl-N-2-pyrimidinylamino)phenyl]-1-piperazinyl}acetamide
N-{2-[(R)-2,6-dioxo-3-piperidyl]-6-fluoro-1-oxo-5-isoindolinyl}{4-[2-chloro-4-(2-pyridylamino)phenyl]-1-piperazinyl}acetamide
N-{2-[(S)-2,6-dioxo-3-piperidyl]-6-fluoro-1-oxo-5-isoindolinyl}{4-[2-chloro-4-(2-pyridylamino)phenyl]-1-piperazinyl}acetamide
N-{2-[(S)-2,6-dioxo-3-piperidyl]-1-oxo-5-isoindolinyl}{4-[4-(N-methyl{5-[2-(methylamino)ethoxy]-2-pyridyl}amino)-2-(trifluoromethyl)phenyl]-1-piperazinyl}acetamide
3-[5-(2-{4-[4-(N-methyl{5-[2-(methylamino)ethoxy]-2-pyridyl}amino)-2-(trifluoromethyl)phenyl]-1-piperazinyl}ethoxy)-1-oxo-2-isoindolinyl]-2,6-piperidinedione
3-(5-{2-[3-(4-{o-[2-(methylamino)ethoxy]phenylamino}-2-(trifluoromethyl)phenyl)-1-azetidinyl]ethoxy}-1-oxo-2-isoindolinyl)-2,6-piperidinedione
3-(5-{2-[3-(2-chloro-4-{o-[2-(methylamino)ethoxy]phenylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}{4-[2-chloro-4-(N-methyl-N-2,4,6-triaza-5-indanylamino)phenyl]-1-piperazinyl}acetamide
N-{2-[(S)-2,6-dioxo-3-piperidyl]-1-oxo-5-isoindolinyl}(4-{2-[(methylamino)methyl]-4-(N-methyl-N-2-pyrimidinylamino)phenyl}-1-piperazinyl)acetamide
N-{2-[(R)-2,6-dioxo-3-piperidyl]-1-oxo-5-isoindolinyl}[3-(2-chloro-4-{o-[2-(methylamino)ethoxy]phenylamino}phenyl)-1-azetidinyl]acetamide
N-{2-[(S)-2,6-dioxo-3-piperidyl]-1-oxo-5-isoindolinyl}{3-[2-chloro-4-(2,4,6-triaza-5-indanylamino)phenyl]-1-azetidinyl}acetamide
N-{2-[(S)-2,6-dioxo-3-piperidyl]-1-oxo-5-isoindolinyl}{3-[2-chloro-4-(m-nitrophenylamino)phenyl]-1-azetidinyl}acetamide
N-{2-[(S)-2,6-dioxo-3-piperidyl]-1-oxo-5-isoindolinyl}{4-[2-hydroxy-4-(N-methyl-N-2-pyrimidinylamino)phenyl]-1-piperazinyl}acetamide
1-methyl 1-{2-[4-({N-2-[(S)-2,6-dioxo-3-piperidyl]-1-oxo-5-isoindolinylcarbamoyl}methyl)-1-piperazinyl]-5-(N-methyl-N-2-pyrimidinylamino)phenyl}methyl phenylmethanecarbamate
3-(5-{2-[3-(2-chloro-4-{p-[2-(methylamino)ethoxy]phenylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}{3-[4-(m-aminophenylamino)-2-chlorophenyl]-1-azetidinyl}acetamide
N-{2-[(S)-2,6-dioxo-3-piperidyl]-1-oxo-5-isoindolinyl}{3-[4-(m-aminophenylamino)-2-chlorophenyl]-1-azetidinyl}acetamide
N-{2-[(S)-2,6-dioxo-3-piperidyl]-1-oxo-5-isoindolinyl}{4-[4-(4-hydroxycyclohexylamino)-2-(trifluoromethyl)phenyl]-1-piperazinyl}acetamide
3-[5-(2-{4-[4-(4-hydroxycyclohexylamino)-2-(trifluoromethyl)phenyl]-1-piperazinyl}ethoxy)-1-oxo-2-isoindolinyl]-2,6-piperidinedione
N-{2-[(S)-2,6-dioxo-3-piperidyl]-1-oxo-5-isoindolinyl}[3-(2-chloro-4-{p-[2-(methylamino)ethoxy]phenylamino}phenyl)-1-azetidinyl]acetamide
N-{2-[(S)-2,6-dioxo-3-piperidyl]-1-oxo-5-isoindolinyl}{3-[4-(o-aminophenylamino)-2-chlorophenyl]-1-azetidinyl}acetamide
N-{2-[(S)-2,6-dioxo-3-piperidyl]-1-oxo-5-isoindolinyl}[3-(3′-hydroxy-4-biphenylyl)-1-azetidinyl]acetamide
N-{2-[(S)-2,6-dioxo-3-piperidyl]-1-oxo-5-isoindolinyl}{3-[4-(N-methyl{p-[2-(methylamino)ethoxy]phenyl}amino)-2-(trifluoromethyl)phenyl]-1-azetidinyl}acetamide
N-{2-[(S)-2,6-dioxo-3-piperidyl]-1-oxo-5-isoindolinyl}{3-[4-(N-methyl{o-[2-(methylamino)ethoxy]phenyl}amino)-2-(trifluoromethyl)phenyl]-1-azetidinyl}acetamide
3-[5-(2-{3-[4-(N-methyl{p-[2-(methylamino)ethoxy]phenyl}amino)-2-(trifluoromethyl)phenyl]-1-azetidinyl}ethoxy)-1-oxo-2-isoindolinyl]-2,6-piperidinedione
3-[5-({3-[3-(cyclohexylamino)-4-tolyl]ureido}methyl)-1-oxo-2-isoindolinyl]-2,6-piperidinedione
3-(5-{2-[3-(3-chloro-3′-hydroxy-4-biphenylyl)-1-azetidinyl]ethoxy}-1-oxo-2-isoindolinyl)-2,6-piperidinedione
3-[5-(2-{3-[4-(N-methyl{o-[2-(methylamino)ethoxy]phenyl}amino)-2-(trifluoromethyl)phenyl]-1-azetidinyl}ethoxy)-1-oxo-2-isoindolinyl]-2,6-piperidinedione
3-[5-(2-{3-[4-(p-aminophenylamino)-2-chlorophenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{3-[2-chloro-4-(N-methyl{o-[2-(methylamino)ethoxy]phenyl}amino)phenyl]-1-azetidinyl}acetamide
N-{2-[(S)-2,6-dioxo-3-piperidyl]-1-oxo-5-isoindolinyl}{3-[4-(p-aminophenylamino)-2-chlorophenyl]-1-azetidinyl}acetamide
3-[5-(2-{3-[2-chloro-4-(4-hydroxycyclohexyl)phenyl]-1-azetidinyl}ethoxy)-1-oxo-2-isoindolinyl]-2,6-piperidinedione
N-[2-(2,6-dioxo-3-piperidyl)-1-oxo-5-isoindolinyl][4-(2-chloro-4-{[2-(methylamino)ethyl]-N-2-pyridylamino}phenyl)-1-piperazinyl]acetamide
3-[5-(2-{3-[2-chloro-4-(N-methyl{o-[2-(methylamino)ethoxy]phenyl}amino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{3-[4-(o-{2-[2-(methylamino)ethoxy]ethyl}phenylamino)-2-(trifluoromethyl)phenyl]-1-azetidinyl}acetamide
3-{5-[2-(4-{2-chloro-4-[(3-hydroxypropyl)-N-2-pyridylamino]phenyl}-1-piperazinyl)ethoxy]-1-oxo-2-isoindolinyl}-2,6-piperidinedione
3-(5-{2-[3-(2-chloro-4-{3-[2-(methylamino)ethoxy]-2-pyridylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}[3-(2-chloro-4-{3-[2-(methylamino)ethoxy]-2-pyridylamino}phenyl)-1-azetidinyl]acetamide
N-{2-[(R)-2,6-dioxo-3-piperidyl]-1-oxo-5-isoindolinyl}{3-[2-chloro-4-(N-methyl{3-[2-(methylamino)ethoxy]-2-pyridyl}amino)phenyl]-1-azetidinyl}acetamide
N-{2-[(S)-2,6-dioxo-3-piperidyl]-1-oxo-5-isoindolinyl}[3-(2-chloro-4-{5-[2-(methylamino)ethoxy]-2-pyridylamino}phenyl)-1-azetidinyl]acetamide
N-{2-[(S)-2,6-dioxo-3-piperidyl]-1-oxo-5-isoindolinyl}{3-[2-chloro-4-(N-methyl{5-[2-(methylamino)ethoxy]-2-pyridyl}amino)phenyl]-1-azetidinyl}acetamide
3-(5-{2-[3-(2-chloro-4-{5-[2-(methylamino)ethoxy]-2-pyridylamino}phenyl)-1-azetidinyl]ethoxy}-1-oxo-2-isoindolinyl)-2,6-piperidinedione
3-[5-(2-{3-[2-chloro-4-(N-methyl{5-[2-(methylamino)ethoxy]-2-pyridyl}amino)phenyl]-1-azetidinyl}ethoxy)-1-oxo-2-isoindolinyl]-2,6-piperidinedione
3-[5-(2-{3-[2-chloro-4-(N-methyl{3-[2-(methylamino)ethoxy]-2-pyridyl}amino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}[3-(4-{5-[2-(methylamino)ethoxy]-2-pyrimidinylamino}-2-(trifluoromethyl)phenyl)-1-azetidinyl]acetamide
N-{2-[(S)-2,6-dioxo-3-piperidyl]-1-oxo-5-isoindolinyl}[3-(4-{5-[2-(methylamino)ethoxy]-2-pyrimidinylamino}-2-(trifluoromethyl)phenyl)-1-azetidinyl]acetamide
N-{2-[(S)-2,6-dioxo-3-piperidyl]-1-oxo-5-isoindolinyl}[3-(2-chloro-4-{5-[2-(methylamino)ethoxy]-2-pyrimidinylamino}phenyl)-1-azetidinyl]acetamide
N-{2-[(S)-2,6-dioxo-3-piperidyl]-1-oxo-5-isoindolinyl}{4-[4-(p-aminophenylamino)-2-(trifluoromethyl)phenyl]-1-piperazinyl}acetamide
N-{2-[(S)-2,6-dioxo-3-piperidyl]-1-oxo-5-isoindolinyl}{4-[4-(m-aminophenylamino)-2-(trifluoromethyl)phenyl]-1-piperazinyl}acetamide
N-{2-[(S)-2,6-dioxo-3-piperidyl]-1-oxo-5-isoindolinyl}{4-[4-(o-aminophenylamino)-2-(trifluoromethyl)phenyl]-1-piperazinyl}acetamide
3-(5-{2-[3-(4-{5-[2-(methylamino)ethoxy]-2-pyrimidinylamino}-2-(trifluoromethyl)phenyl)-1-azetidinyl]ethoxy}-1-oxo-2-isoindolinyl)-2,6-piperidinedione
3-(5-{2-[3-(2-chloro-4-{5-[2-(methylamino)ethoxy]-2-pyrimidinylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}[3-(2-chloro-4-{[2-(methylamino)ethyl]-N-2-pyridylamino}phenyl)-1-azetidinyl]acetamide
N-{2-[(S)-2,6-dioxo-3-piperidyl]-1-oxo-5-isoindolinyl}{4-[2-chloro-4-(N-methyl{5-[2-(methylamino)ethoxy]-2-pyrimidinyl}amino)phenyl]-1-piperazinyl}acetamide
N-{2-[(S)-2,6-dioxo-3-piperidyl]-1-oxo-5-isoindolinyl}{4-[2-chloro-4-(N-methyl{5-[2-(methylamino)ethoxy]-2-pyridyl}amino)phenyl]-1-piperazinyl}acetamide
N-{2-[(S)-2,6-dioxo-3-piperidyl]-1-oxo-5-isoindolinyl}{4-[2-chloro-4-(N-methyl{p-[2-(methylamino)ethoxy]phenyl}amino)phenyl]-1-piperazinyl}acetamide
3-[5-(2-{3-[2-chloro-4-(N-methyl{p-[2-(methylamino)ethoxy]phenyl}amino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{3-[p-(N-methyl{p-[2-(methylamino)ethoxy]phenyl}amino)phenyl]-1-azetidinyl}acetamide
3-(5-{2-[3-(2-methoxy-4-{p-[2-(methylamino)ethoxy]phenylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}[3-(2-methoxy-4-{p-[2-(methylamino)ethoxy]phenylamino}phenyl)-1-azetidinyl]acetamide
N-{2-[(R)-2,6-dioxo-3-piperidyl]-1-oxo-5-isoindolinyl}{4-[2-chloro-4-(N-methyl{o-[2-(methylamino)ethoxy]phenyl}amino)phenyl]-1-piperazinyl}acetamide
N-{2-[(S)-2,6-dioxo-3-piperidyl]-1-oxo-5-isoindolinyl}{4-[2-chloro-4-(N-methyl{3-[2-(methylamino)ethoxy]-2-pyridyl}amino)phenyl]-1-piperazinyl}acetamide
N-{2-[(R)-2,6-dioxo-3-piperidyl]-1-oxo-5-isoindolinyl}[3-(2-methoxy-4-{o-[2-(methylamino)ethoxy]phenylamino}phenyl)-1-azetidinyl]acetamide
3-(5-{2-[3-(2-methoxy-4-{o-[2-(methylamino)ethoxy]phenylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}{3-[2-chloro-4-(N-methyl{p-[2-(methylamino)ethoxy]phenyl}amino)phenyl]-1-azetidinyl}acetamide
N-[2-(2,6-dioxo-3-piperidyl)-1-oxo-5-isoindolinyl]{4-[2-chloro-4-(4-hydroxy-1-piperidyl)phenyl]-1-piperazinyl}acetamide
N-{2-[(S)-2,6-dioxo-3-piperidyl]-1-oxo-5-isoindolinyl}{3-[2-chloro-4-(p-{2-[2-(methylamino)ethoxy]ethyl}phenylamino)phenyl]-1-azetidinyl}acetamide
N-{2-[(R)-2,6-dioxo-3-piperidyl]-1-oxo-5-isoindolinyl}(3-{2-chloro-4-[N-methyl(p-{2-[2-(methylamino)ethoxy]ethyl}phenyl)amino]phenyl}-1-azetidinyl)acetamide
3-{5-[2-(3-{2-chloro-4-[N-methyl(p-{2-[2-(methylamino)ethoxy]ethyl}phenyl)amino]phenyl}-1-azetidinyl)ethoxy]-1-oxo-2-isoindolinyl}-2,6-piperidinedione
3-{5-[2-(3-{2-chloro-4-[N-methyl(p-{2-[2-(methylamino)ethoxy]ethyl}phenyl)amino]phenyl}-1-azetidinyl)ethoxy]-1-oxo-2-isoindolinyl}-2,6-piperidinedione
3-[5-(2-{3-[2-chloro-4-(p-{2-[2-(methylamino)ethoxy]ethyl}phenylamino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{3-[4-(4-hydroxy-1-piperidyl)-2-(trifluoromethyl)phenyl]-1-azetidinyl}acetamide
N-{2-[(S)-2,6-dioxo-3-piperidyl]-1-oxo-5-isoindolinyl}{3-[2-chloro-4-(4-hydroxy-1-piperidyl)phenyl]-1-azetidinyl}acetamide
3-[5-(2-{3-[2-chloro-4-(4-hydroxy-1-piperidyl)phenyl]-1-azetidinyl}ethoxy)-1-oxo-2-isoindolinyl]-2,6-piperidinedione
3-[5-(2-{3-[4-(4-hydroxy-1-piperidyl)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}[4-(4-{[5-(2-aminoethoxy)-2-pyrimidinyl]-N-methylamino}-2-chlorophenyl)-1-piperazinyl]acetamide
N-{2-[(S)-2,6-dioxo-3-piperidyl]-1-oxo-5-isoindolinyl}{3-[4-(4-hydroxy-4-methyl-1-piperidyl)-2-(trifluoromethyl)phenyl]-1-azetidinyl}acetamide
3-[5-(2-{3-[4-(4-hydroxy-4-methyl-1-piperidyl)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{4-[4-(4-amino-1-piperidyl)-2-chlorophenyl]-1-piperazinyl}acetamide
N-{2-[(R)-2,6-dioxo-3-piperidyl]-1-oxo-5-isoindolinyl}{3-[4-(3-ethyl-3-hydroxy-1-azetidinyl)-2-(trifluoromethyl)phenyl]-1-azetidinyl}acetamide
3-[5-(2-{3-[4-(3-ethyl-3-hydroxy-1-azetidinyl)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{4-[2-chloro-4-(N-methyl{6-[2-(methylamino)ethoxy]-3-pyridazinyl}amino)phenyl]-1-piperazinyl}acetamide
N-{2-[(S)-2,6-dioxo-3-piperidyl]-1-oxo-5-isoindolinyl}{3-[4-(3-hydroxy-3-methyl-1-azetidinyl)-2-(trifluoromethyl)phenyl]-1-azetidinyl}acetamide
3-[5-(2-{3-[4-(3-hydroxy-3-methyl-1-azetidinyl)-2-(trifluoromethyl)phenyl]-1-azetidinyl}ethoxy)-1-oxo-2-isoindolinyl]-2,6-piperidinedione
N-(2-{[3-chloro-4-(4-{2-[2-(2,6-dioxo-3-piperidyl)-1-oxo-5-isoindolinyloxy]ethyl}-1-piperazinyl)phenyl]-N-2-pyridylamino}ethyl)-N-methyl(R)-lactamide
N-{2-[2-({3-chloro-4-[4-({N-2-[(R)-2,6-dioxo-3-piperidyl]-1-oxo-5-isoindolinylcarbamoyl}methyl)-1-piperazinyl]phenyl}-N-methylamino)-5-pyrimidinyloxy]ethyl}-N-methylglycolamide
3-(5-{2-[4-(2-chloro-4-{[2-(2-hydroxyethoxy)ethyl]-N-2-pyridylamino}phenyl)-1-piperazinyl]ethoxy}-1-oxo-2-isoindolinyl)-2,6-piperidinedione
N-{2-[6-({3-chloro-4-[4-({N-2-[(R)-2,6-dioxo-3-piperidyl]-1-oxo-5-isoindolinylcarbamoyl}methyl)-1-piperazinyl]phenyl}-N-methylamino)-3-pyridyloxy]ethyl}-N-methylglycolamide
N-{2-[(S)-2,6-dioxo-3-piperidyl]-1-oxo-5-isoindolinyl}(4-{2-chloro-4-[4-(methylamino)-1-piperidyl]phenyl}-1-piperazinyl)acetamide
N-{2-[(S)-2,6-dioxo-3-piperidyl]-1-oxo-5-isoindolinyl}{4-[2-chloro-4-(N-methyl{5-[2-(methylamino)ethoxy]-2-pyrazinyl}amino)phenyl]-1-piperazinyl}acetamideâ€″formic acid (1/2)
N-{2-[(S)-2,6-dioxo-3-piperidyl]-1-oxo-5-isoindolinyl}[4-(4-{[5-(2-aminoethoxy)-2-pyridyl]-N-methylamino}-2-chlorophenyl)-1-piperazinyl]acetamide
N-{2-[(S)-2,6-dioxo-3-piperidyl]-1-oxo-5-isoindolinyl}[4-(2-chloro-4-{N-methyl[m-(3-methyl-6,9-dioxa-3-azadecyloxy)phenyl]amino}phenyl)-1-piperazinyl]acetamide
N-{2-[(S)-2,6-dioxo-3-piperidyl]-1-oxo-5-isoindolinyl}{3-[2-chloro-4-(4-hydroxy-4-methyl-1-piperidyl)phenyl]-1-azetidinyl}acetamide
N-{2-[(S)-2,6-dioxo-3-piperidyl]-1-oxo-5-isoindolinyl}[4-(2-chloro-4-{N-methyl[m-(3-methyl-6,9,12,15-tetraoxa-3-azahexadecyloxy)phenyl]amino}phenyl)-1-piperazinyl]acetamide
3-[5-(2-{3-[2-chloro-4-(4-hydroxy-4-methyl-1-piperidyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}[4-(2-chloro-4-{N-methyl[m-(3-methyl-6,9,12-trioxa-3-azatridecyloxy)phenyl]amino}phenyl)-1-piperazinyl]acetamide
N-{2-[(R)-2,6-dioxo-3-piperidyl]-1-oxo-5-isoindolinyl}{4-[2-chloro-4-(N-methyl{3-[2-(methylamino)ethoxy]-2-pyrazinyl}amino)phenyl]-1-piperazinyl}acetamide
N-{2-[(S)-2,6-dioxo-3-piperidyl]-1-oxo-5-isoindolinyl}[4-(2-chloro-4-{N-methyl[m-(3-methyl-6,9,12,15,18-pentaoxa-3-azanonadecyloxy)phenyl]amino}phenyl)-1-piperazinyl]acetamide
3-[5-(2-{4-[2-chloro-4-(N-methyl{5-[2-(methylamino)ethoxy]-2-pyrimidinyl}amino)phenyl]-1-piperazinyl}ethoxy)-1-oxo-2-isoindolinyl]-2,6-piperidinedione
3-[5-(2-{4-[2-chloro-4-(N-methyl{5-[2-(methylamino)ethoxy]-2-pyridyl}amino)phenyl]-1-piperazinyl}ethoxy)-1-oxo-2-isoindolinyl]-2,6-piperidinedione
N-{2-[m-({3-chloro-4-[4-({N-2-[(R)-2,6-dioxo-3-piperidyl]-1-oxo-5-isoindolinylcarbamoyl}methyl)-1-piperazinyl]phenyl}-N-methylamino)phenoxy]ethyl}-N-methyl(2-methoxyethoxy)acetamide
N-{2-[m-({3-chloro-4-[4-({N-2-[(R)-2,6-dioxo-3-piperidyl]-1-oxo-5-isoindolinylcarbamoyl}methyl)-1-piperazinyl]phenyl}-N-methylamino)phenoxy]ethyl}-N-methyl(2-methoxyethoxy)acetamide
N-{2-[m-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethyl}-N-methyl3,6,9-trioxadecanamide
N-{2-[m-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethyl}-N-methyl3,6,9,12-tetraoxatridecanamide
N-{2-[m-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethyl}-N-methyl3,6,9,12,15-pentaoxahexadecanamide
N-{2-[(R)-2,6-dioxo-3-piperidyl]-1-oxo-5-isoindolinyl}{4-[2-chloro-4-(N-methyl{5-[2-(methylamino)ethoxy]-4-pyrimidinyl}amino)phenyl]-1-piperazinyl}acetamide
3-[5-(2-{4-[2-chloro-4-(N-methyl{p-[2-(methylamino)ethoxy]phenyl}amino)phenyl]-1-piperazinyl}ethoxy)-1-oxo-2-isoindolinyl]-2,6-piperidinedione
3-[5-(2-{3-[4-(4-methoxy-1-piperidyl)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}(3-{p-[N-(Methyl-d3)(p-{2-[2-(methylamino)ethoxy]ethyl}phenyl)amino]phenyl}-1-azetidinyl)acetamide
N-{2-[(S)-2,6-dioxo-3-piperidyl]-1-oxo-5-isoindolinyl}(3-{2-chloro-4-[N-(Methyl-d3)(p-{2-[2-(methylamino)ethoxy]ethyl}phenyl)amino]phenyl}-1-azetidinyl)acetamide
3-{5-[2-(3-{2-chloro-4-[N-(²H₃)methyl(p-{2-[2-(methylamino)ethoxy]ethyl}phenyl)amino]phenyl}-1-azetidinyl)ethoxy]-1-oxo-2-isoindolinyl}-2,6-piperidinedione
N-{2-[2-(p-{[3-chloro-4-(1-{2-[2-(2,6-dioxo-3-piperidyl)-1-oxo-5-isoindolinyloxy]ethyl}-3-azetidinyl)phenyl]-N-methylamino}phenyl)ethoxy]ethyl}-N-methylglycolamide
N-{2-[(S)-2,6-dioxo-3-piperidyl]-1-oxo-5-isoindolinyl}(4-{2-chloro-4-[N-(Methyl-d3){5-[2-(methylamino)ethoxy]-2-pyrimidinyl}amino]phenyl}-1-piperazinyl)acetamide
N-{2-[(S)-2,6-dioxo-3-piperidyl]-1-oxo-5-isoindolinyl}(4-{2-chloro-4-[N-(Methyl-d3){5-[2-(methylamino)ethoxy]-2-pyrimidinyl}amino]phenyl}-1-piperazinyl)acetamide
(S)-3-{5-[2-(4-{2-chloro-4-[N-(Methyl-d3){5-[2-(methylamino)ethoxy]-2-pyrimidinyl}amino]phenyl}-1-piperazinyl)ethoxy]-1-oxo-2-isoindolinyl}-2,6-piperidinedione
3-(5-{2-[3-(2-chloro-4-{[2-(methylamino)ethyl]-N-2-pyridylamino}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}{3-[4-(5-{2-[2-(methylamino)ethoxy]ethyl}-2-pyridylamino)-2-(trifluoromethyl)phenyl]-1-azetidinyl}acetamide
N-{2-[(S)-2,6-dioxo-3-piperidyl]-1-oxo-5-isoindolinyl}[4-(4-{[p-(2-aminoethoxy)phenyl]-N-methylamino}-2-chlorophenyl)-1-piperazinyl]acetamide
3-[5-(2-{3-[4-(5-{2-[2-(methylamino)ethoxy]ethyl}-2-pyridylamino)-2-(trifluoromethyl)phenyl]-1-azetidinyl}ethoxy)-1-oxo-2-isoindolinyl]-2,6-piperidinedione
N-{2-[m-({3-chloro-4-[4-({N-2-[(R)-2,6-dioxo-3-piperidyl]-1-oxo-5-isoindolinylcarbamoyl}methyl)-1-piperazinyl]phenyl}-N-methylamino)phenoxy]ethyl}-N-methyl3-mercaptopropionamide
N-{2-[m-({3-chloro-4-[4-({N-2-[(R)-2,6-dioxo-3-piperidyl]-1-oxo-5-isoindolinylcarbamoyl}methyl)-1-piperazinyl]phenyl}-N-methylamino)phenoxy]ethyl}-N-methylmercaptoacetamide
3-(5-{2-[4-(4-{[2-(methylamino)ethyl]-N-2-pyridylamino}-2-(trifluoromethyl)phenyl)-1-piperazinyl]ethoxy}-1-oxo-2-isoindolinyl)-2,6-piperidinedione
N-(2-{[3-chloro-4-(1-{2-[2-(2,6-dioxo-3-piperidyl)-1-oxo-5-isoindolinyloxy]ethyl}-3-azetidinyl)phenyl]-N-2-pyridylamino}ethyl)-N-methylglycolamide
N-[2-(6-{[4-(4-{2-[2-(2,6-dioxo-3-piperidyl)-1-oxo-5-isoindolinyloxy]ethyl}-1-piperazinyl)-3-(trifluoromethyl)phenyl]-N-methylamino}-3-pyridyloxy)ethyl]-N-methylglycolamide
N-[2-({3-chloro-4-[1-({N-2-[(S)-2,6-dioxo-3-piperidyl]-1-oxo-5-isoindolinylcarbamoyl}methyl)-3-azetidinyl]phenyl}-N-2-pyridylamino)ethyl]-N-methylglycolamide
N-{2-[(S)-2,6-dioxo-3-piperidyl]-1-oxo-5-isoindolinyl}{3-[4-(p-{2-[2-(methylamino)ethoxy]ethoxy}phenylamino)-2-(trifluoromethyl)phenyl]-1-azetidinyl}acetamide
N-{2-[(S)-2,6-dioxo-3-piperidyl]-1-oxo-5-isoindolinyl}(4-{2-chloro-4-[N-(Methyl-d3){p-[2-(methylamino)ethoxy]phenyl}amino]phenyl}-1-piperazinyl)acetamide
N-{2-[(S)-2,6-dioxo-3-piperidyl]-1-oxo-5-isoindolinyl}(4-{2-chloro-4-[5-(2-methoxyethoxy)-2-pyrimidinylamino]phenyl}-1-piperazinyl)acetamide
3-[5-(2-{3-[4-(p-{2-[2-(methylamino)ethoxy]ethoxy}phenylamino)-2-(trifluoromethyl)phenyl]-1-azetidinyl}ethoxy)-1-oxo-2-isoindolinyl]-2,6-piperidinedione
3-[5-(2-{4-[2-chloro-4-(di-2-pyridylamino)phenyl]-1-piperazinyl}ethoxy)-1-oxo-2-isoindolinyl]-2,6-piperidinedione
3-(5-{2-[3-(2-chloro-4-{4-methyl-4-[2-(methylamino)ethoxy]-1-piperidyl}phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}[4-(2-chloro-4-{[5-(2-hydroxyethoxy)-2-pyrimidinyl]-N-(Methyl-d3)amino}phenyl)-1-piperazinyl]acetamide
N-{2-[(S)-2,6-dioxo-3-piperidyl]-1-oxo-5-isoindolinyl}[4-(2-chloro-4-{[5-(2-hydroxyethoxy)-2-pyrimidinyl]-N-(²H₃)methylamino}phenyl)-1-piperazinyl]acetamide
N-{2-[(S)-2,6-dioxo-3-piperidyl]-1-oxo-5-isoindolinyl}{4-[2-chloro-4-(p-{2-[2-(methylamino)ethoxy]ethoxy}phenylamino)phenyl]-1-piperazinyl}acetamide
N-{2-[(S)-2,6-dioxo-3-piperidyl]-1-oxo-5-isoindolinyl}(4-{2-chloro-4-[N-(Methyl-d3)(p-{2-[2-(methylamino)ethoxy]ethoxy}phenyl)amino]phenyl}-1-piperazinyl)acetamide
N-{2-[(S)-2,6-dioxo-3-piperidyl]-1-oxo-5-isoindolinyl}[3-(2-chloro-4-{4-methyl-4-[2-(methylamino)ethoxy]-1-piperidyl}phenyl)-1-azetidinyl]acetamide
N-{2-[(S)-2,6-dioxo-3-piperidyl]-1-oxo-5-isoindolinyl}[3-(2-chloro-4-{4-[2-(methylamino)ethoxy]-1-piperidyl}phenyl)-1-azetidinyl]acetamide
N-{2-[2-({3-chloro-4-[4-({N-2-[(S)-2,6-dioxo-3-piperidyl]-1-oxo-5-isoindolinylcarbamoyl}methyl)-1-piperazinyl]phenyl}-N-(Methyl-d3)amino)-5-pyrimidinyloxy]ethyl}-N-methylglycolamide
N-{2-[(S)-2,6-dioxo-3-piperidyl]-1-oxo-5-isoindolinyl}(4-{2-chloro-4-[N-(Methyl-d3)(m-{2-[2-(methylamino)ethoxy]ethyl}phenyl)amino]phenyl}-1-piperazinyl)acetamide
N-{2-[(S)-2,6-dioxo-3-piperidyl]-1-oxo-5-isoindolinyl}{3-[2-chloro-4-(m-{2-[2-(methylamino)ethoxy]ethyl}phenylamino)phenyl]-1-azetidinyl}acetamide
N-(2-{2-[p-({3-chloro-4-[1-({N-2-[(R)-2,6-dioxo-3-piperidyl]-1-oxo-5-isoindolinylcarbamoyl}methyl)-3-azetidinyl]phenyl}-N-methylamino)phenyl]ethoxy}ethyl)-N-methylglycolamide
3-[5-(2-{3-[2-chloro-4-(m-{2-[2-(methylamino)ethoxy]ethyl}phenylamino)phenyl]-1-azetidinyl}ethoxy)-1-oxo-2-isoindolinyl]-2,6-piperidinedione
3-[5-(2-{3-[4-(m-{2-[2-(methylamino)ethoxy]ethyl}phenylamino)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{4-[2-chloro-4-({5-[2-(2-methoxyethylamino)ethoxy]-2-pyrimidinyl}-N-(²H₃)methylamino)phenyl]-1-piperazinyl}acetamide
N-{2-[(S)-2,6-dioxo-3-piperidyl]-1-oxo-5-isoindolinyl}{3-[4-(m-{2-[2-(methylamino)ethoxy]ethyl}phenylamino)-2-(trifluoromethyl)phenyl]-1-azetidinyl}acetamide
N-(2-{[4-(4-{2-[2-(2,6-dioxo-3-piperidyl)-1-oxo-5-isoindolinyloxy]ethyl}-1-piperazinyl)-3-(trifluoromethyl)phenyl]-N-2-pyridylamino}ethyl)-N-methylglycolamide
N-[2-(2-{m-[4-(1-{2-[2-(2,6-dioxo-3-piperidyl)-1-oxo-5-isoindolinyloxy]ethyl}-3-azetidinyl)-3-(trifluoromethyl)phenylamino]phenyl}ethoxy)ethyl]-N-methylglycolamide
3-[5-(2-{3-[4-(4-amino-1-piperidyl)-2-(trifluoromethyl)phenyl]-1-azetidinyl}ethoxy)-1-oxo-2-isoindolinyl]-2,6-piperidinedione
N-{2-[(R)-2,6-dioxo-3-piperidyl]-1-oxo-5-isoindolinyl}{3-[4-(4-amino-1-piperidyl)-2-(trifluoromethyl)phenyl]-1-azetidinyl}acetamide
3-[5-(2-{4-[4-(4-mercapto-1-piperidyl)-2-(trifluoromethyl)phenyl]-1-piperazinyl}ethoxy)-1-oxo-2-isoindolinyl]-2,6-piperidinedione
3-{5-[2-(3-{4-[4-(methylamino)-1-piperidyl]-2-(trifluoromethyl)phenyl}-1-azetidinyl)ethoxy]-1-oxo-2-isoindolinyl}-2,6-piperidinedione
C-methyl 1-[4-(4-{2-[2-(2,6-dioxo-3-piperidyl)-1-oxo-5-isoindolinyloxy]ethyl}-1-piperazinyl)-3-(trifluoromethyl)phenyl]-4-piperidinecarbothioate
C-methyl 1-{3-chloro-4-[4-({N-2-[(R)-2,6-dioxo-3-piperidyl]-1-oxo-5-isoindolinylcarbamoyl}methyl)-1-piperazinyl]phenyl}-4-piperidinecarbothioate
C-methyl 1-{4-[4-({N-2-[(S)-2,6-dioxo-3-piperidyl]-1-oxo-5-isoindolinylcarbamoyl}methyl)-1-piperazinyl]-3-(trifluoromethyl)phenyl}-4-piperidinecarbothioate
N-{2-[(R)-2,6-dioxo-3-piperidyl]-1-oxo-5-isoindolinyl}(3-{4-[N-methyl(m-{2-[2-(methylamino)ethoxy]ethyl}phenyl)amino]-2-(trifluoromethyl)phenyl}-1-azetidinyl)acetamide
3-{5-[2-(3-{4-[N-methyl(m-{2-[2-(methylamino)ethoxy]ethyl}phenyl)amino]-2-(trifluoromethyl)phenyl}-1-azetidinyl)ethoxy]-1-oxo-2-isoindolinyl}-2,6-piperidinedione
N-{2-[(R)-2,6-dioxo-3-piperidyl]-1-oxo-5-isoindolinyl}(3-{4-[4-(methylamino)-1-piperidyl]-2-(trifluoromethyl)phenyl}-1-azetidinyl)acetamide
3-{5-[2-(3-{4-[4-(2-methoxyethoxy)-1-piperidyl]-2-(trifluoromethyl)phenyl}-1-azetidinyl)ethoxy]-1-oxo-2-isoindolinyl}-2,6-piperidinedione
N-{2-[(S)-2,6-dioxo-3-piperidyl]-1-oxo-5-isoindolinyl}{3-[2-chloro-4-({5-[2-(2-methoxyethylamino)ethoxy]-2-pyrimidinyl}-N-(Methyl-d3)amino)phenyl]-1-azetidinyl}acetamide
N-{2-[(S)-2,6-dioxo-3-piperidyl]-1-oxo-5-isoindolinyl}[4-(4-{5-[2-(methylamino)ethoxy]-2-pyrimidinylamino}-2-(trifluoromethyl)phenyl)-1-piperazinyl]acetamide
(S)-3-{5-[2-(4-{2-chloro-4-[5-(2-methoxyethoxy)-2-pyrimidinylamino]phenyl}-1-piperazinyl)ethoxy]-1-oxo-2-isoindolinyl}-2,6-piperidinedione
3-(5-{2-[3-(4-{4-[2-(2-methoxyethoxy)ethoxy]-1-piperidyl}-2-(trifluoromethyl)phenyl)-1-azetidinyl]ethoxy}-1-oxo-2-isoindolinyl)-2,6-piperidinedione
3-[5-(2-{3-[4-(4-{2-[2-(2-methoxyethoxy)ethoxy]ethoxy}-1-piperidyl)-2-(trifluoromethyl)phenyl]-1-azetidinyl}ethoxy)-1-oxo-2-isoindolinyl]-2,6-piperidinedione
N-(2-{2-[p-({3-chloro-4-[1-({N-2-[(S)-2,6-dioxo-3-piperidyl]-1-oxo-5-isoindolinylcarbamoyl}methyl)-3-azetidinyl]phenyl}-N-methylamino)phenyl]ethoxy}ethyl)-N-methyl3,6,9-trioxadecanamide
N-(2-{2-[p-({3-chloro-4-[1-({N-2-[(R)-2,6-dioxo-3-piperidyl]-1-oxo-5-isoindolinylcarbamoyl}methyl)-3-azetidinyl]phenyl}-N-methylamino)phenyl]ethoxy}ethyl)-N-methyl(2-methoxyethoxy)acetamide
N-{2-[(R)-2,6-dioxo-3-piperidyl]-1-oxo-5-isoindolinyl}[4-(2-chloro-4-{N-methyl[m-(3-methyl-6,9-dioxa-3-azadecyloxy)phenyl]amino}phenyl)-1-piperazinyl]acetamide
N-{2-[(R)-2,6-dioxo-3-piperidyl]-1-oxo-5-isoindolinyl}{4-[4-(5-acetyl-2-pyridylamino)-2-chlorophenyl]-1-piperazinyl}acetamide
3-[5-(2-{3-[4-(p-{2-[3-(methylamino)propoxy]ethyl}phenylamino)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{4-[4-(p-acetylphenylamino)-2-chlorophenyl]-1-piperazinyl}acetamide
N-{2-[(S)-2,6-dioxo-3-piperidyl]-1-oxo-5-isoindolinyl}{4-[4-(4-acetyl-2-pyridylamino)-2-chlorophenyl]-1-piperazinyl}acetamide
N-{2-[(S)-2,6-dioxo-3-piperidyl]-1-oxo-5-isoindolinyl}{4-[4-(m-acetylphenylamino)-2-chlorophenyl]-1-piperazinyl}acetamide
N-{2-[(S)-2,6-dioxo-3-piperidyl]-1-oxo-5-isoindolinyl}[4-(2-chloro-4-{p-[2-(2-methoxyethoxy)ethoxy]phenylamino}phenyl)-1-piperazinyl]acetamide
N-{2-[(S)-2,6-dioxo-3-piperidyl]-1-oxo-5-isoindolinyl}[3-(2-chloro-4-{p-[2-(2-methoxyethoxy)ethoxy]phenylamino}phenyl)-1-azetidinyl]acetamide
N-{2-[(S)-2,6-dioxo-3-piperidyl]-1-oxo-5-isoindolinyl}{4-[2-chloro-4-(p-{2-[2-(2-methoxyethoxy)ethoxy]ethoxy}phenylamino)phenyl]-1-piperazinyl}acetamide
N-{2-[(S)-2,6-dioxo-3-piperidyl]-1-oxo-5-isoindolinyl}{3-[2-chloro-4-(p-{2-[2-(2-methoxyethoxy)ethoxy]ethoxy}phenylamino)phenyl]-1-azetidinyl}acetamide
(S)-3-(5-{2-[4-(2-chloro-4-{p-[2-(2-methoxyethoxy)ethoxy]phenylamino}phenyl)-1-piperazinyl]ethoxy}-1-oxo-2-isoindolinyl)-2,6-piperidinedione
(S)-3-(5-{2-[3-(2-chloro-4-{p-[2-(2-methoxyethoxy)ethoxy]phenylamino}phenyl)-1-azetidinyl]ethoxy}-1-oxo-2-isoindolinyl)-2,6-piperidinedione
(S)-3-[5-(2-{4-[2-chloro-4-(p-{2-[2-(2-methoxyethoxy)ethoxy]ethoxy}phenylamino)phenyl]-1-piperazinyl}ethoxy)-1-oxo-2-isoindolinyl]-2,6-piperidinedione
(S)-3-[5-(2-{3-[2-chloro-4-(p-{2-[2-(2-methoxyethoxy)ethoxy]ethoxy}phenylamino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{3-[4-(1-oxa-4,9-diaza-9-spiro[5.5]undecyl)-2-(trifluoromethyl)phenyl]-1-azetidinyl}acetamide
3-[5-(2-{3-[4-(1-oxa-4,9-diaza-9-spiro[5.5]undecyl)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}[4-(2-chloro-4-{5-[2-(methylamino)ethoxy]-2-pyrimidinylamino}phenyl)-1-piperazinyl]acetamide
N-{2-[(S)-2,6-dioxo-3-piperidyl]-1-oxo-5-isoindolinyl}(3-{4-[N-(Methyl-d3)(p-{2-[3-(methylamino)propoxy]ethyl}phenyl)amino]-2-(trifluoromethyl)phenyl}-1-azetidinyl)acetamide
3-{5-[2-(3-{4-[N-(Methyl-d3)(p-{2-[3-(methylamino)propoxy]ethyl}phenyl)amino]-2-(trifluoromethyl)phenyl}-1-azetidinyl)ethoxy]-1-oxo-2-isoindolinyl}-2,6-piperidinedione
N-{2-[(S)-2,6-dioxo-3-piperidyl]-1-oxo-5-isoindolinyl}{3-[2-chloro-4-({2-[2-(methylamino)ethoxy]ethyl}-N-2-pyridylamino)phenyl]-1-azetidinyl}acetamide
3-[5-(2-{3-[2-chloro-4-({2-[2-(methylamino)ethoxy]ethyl}-N-2-pyridylamino)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}(4-{4-[N-methyl(5-{2-[2-(methylamino)ethoxy]ethyl}-2-pyrimidinyl)amino]-2-(trifluoromethyl)phenyl}-1-piperazinyl)acetamide
C-methyl [2-(N-methyl{4-[4-({N-2-[(S)-2,6-dioxo-3-piperidyl]-1-oxo-5-isoindolinylcarbamoyl}methyl)-1-piperazinyl]-3-(trifluoromethyl)phenyl}amino)-4-pyrimidinyl]ethanethioate
N-{2-[(S)-2,6-dioxo-3-piperidyl]-1-oxo-5-isoindolinyl}{3-[2-chloro-4-(5-{2-[2-(methylamino)ethoxy]ethyl}-2-pyrimidinylamino)phenyl]-1-azetidinyl}acetamide
3-[5-(2-{3-[2-chloro-4-(5-{2-[2-(methylamino)ethoxy]ethyl}-2-pyrimidinylamino)phenyl]-1-azetidinyl}ethoxy)-1-oxo-2-isoindolinyl]-2,6-piperidinedione
(S)-3-[5-(2-{4-[2-chloro-4-(5-{2-[2-(methylamino)ethoxy]ethyl}-2-pyrimidinylamino)phenyl]-1-piperazinyl}ethoxy)-1-oxo-2-isoindolinyl]-2,6-piperidinedione
N-{2-[(S)-2,6-dioxo-3-piperidyl]-1-oxo-5-isoindolinyl}{4-[2-chloro-4-({m-[2-(2-fluoroethylamino)ethoxy]phenyl}-N-methylamino)phenyl]-1-piperazinyl}acetamide
N-{2-[(S)-2,6-dioxo-3-piperidyl]-1-oxo-5-isoindolinyl}{4-[2-chloro-4-({m-[2-(2,2-difluoroethylamino)ethoxy]phenyl}-N-methylamino)phenyl]-1-piperazinyl}acetamide
N-{2-[(S)-2,6-dioxo-3-piperidyl]-1-oxo-5-isoindolinyl}(3-{2-chloro-4-[N-(Methyl-d3){5-[2-(methylamino)ethoxy]-2-pyrimidinyl}amino]phenyl}-1-azetidinyl)acetamide
N-{2-[(S)-2,6-dioxo-3-piperidyl]-1-oxo-5-isoindolinyl}{4-[2-chloro-4-(5-{2-[2-(methylamino)ethoxy]ethyl}-2-pyrimidinylamino)phenyl]-1-piperazinyl}acetamide
N-{2-[(R)-2,6-dioxo-3-piperidyl]-1-oxo-5-isoindolinyl}(3-{2-chloro-4-[N-(Methyl-d3)(5-{2-[2-(methylamino)ethoxy]ethyl}-2-pyrimidinyl)amino]phenyl}-1-azetidinyl)acetamide
3-{5-[2-(3-{2-chloro-4-[N-(Methyl-d3)(5-{2-[2-(methylamino)ethoxy]ethyl}-2-pyrimidinyl)amino]phenyl}-1-azetidinyl)ethoxy]-1-oxo-2-isoindolinyl}-2,6-piperidinedione
N-methyl-N-(2-{2-[2-(N-methyl{4-[4-({N-2-[(R)-2,6-dioxo-3-piperidyl]-1-oxo-5-isoindolinylcarbamoyl}methyl)-1-piperazinyl]-3-(trifluoromethyl)phenyl}amino)-5-pyrimidinyl]ethoxy}ethyl)glycolamide
3-[5-(2-{3-[4-(4-glycoloyl-1-oxa-4,9-diaza-9-spiro[5.5]undecyl)-2-(trifluoromethyl)phenyl]-1-azetidinyl}ethoxy)-1-oxo-2-isoindolinyl]-2,6-piperidinedione
N-{2-[(S)-2,6-dioxo-3-piperidyl]-1-oxo-5-isoindolinyl}{4-[2-chloro-4-({m-[2-(ethylamino)ethoxy]phenyl}-N-methylamino)phenyl]-1-piperazinyl}acetamide
3-[5-(2-{4-[2-chloro-4-({m-[2-(ethylamino)ethoxy]phenyl}-N-methylamino)phenyl]-1-piperazinyl}ethoxy)-1-oxo-2-isoindolinyl]-2,6-piperidinedione
N-{2-[(S)-2,6-dioxo-3-piperidyl]-1-oxo-5-isoindolinyl}(3-{2-chloro-4-[4-(methylamino)-1-piperidyl]phenyl}-1-azetidinyl)acetamide
N-{2-[(S)-2,6-dioxo-3-piperidyl]-1-oxo-5-isoindolinyl}(4-{2-chloro-4-[N-(Methyl-d3)(5-{2-[2-(methylamino)ethoxy]ethyl}-2-pyrimidinyl)amino]phenyl}-1-piperazinyl)acetamide
3-{5-[2-(4-{2-chloro-4-[N-(Methyl-d3)(5-{2-[2-(methylamino)ethoxy]ethyl}-2-pyrimidinyl)amino]phenyl}-1-piperazinyl)ethoxy]-1-oxo-2-isoindolinyl}-2,6-piperidinedione
N-{3-[2-(p-{[4-(1-{2-[2-(2,6-dioxo-3-piperidyl)-1-oxo-5-isoindolinyloxy]ethyl}-3-azetidinyl)-3-(trifluoromethyl)phenyl]-N-(Methyl-d3)amino}phenyl)ethoxy]propyl}-N-methylglycolamide
3-{5-[2-(3-{2-chloro-4-[4-(methylamino)-1-piperidyl]phenyl}-1-azetidinyl)ethoxy]-1-oxo-2-isoindolinyl}-2,6-piperidinedione
N-{2-[(S)-2,6-dioxo-3-piperidyl]-1-oxo-5-isoindolinyl}[4-(2-chloro-4-{5-[2-(1-piperazinyl)ethoxy]-2-pyrimidinylamino}phenyl)-1-piperazinyl]acetamide
N-{2-[(S)-2,6-dioxo-3-piperidyl]-1-oxo-5-isoindolinyl}[4-(2-chloro-4-{5-[2-(isopropylamino)ethoxy]-2-pyrimidinylamino}phenyl)-1-piperazinyl]acetamide
N-{2-[(S)-2,6-dioxo-3-piperidyl]-1-oxo-5-isoindolinyl}{4-[2-chloro-4-(5-{[1-(methylamino)cyclopropyl]methoxy}-2-pyrimidinylamino)phenyl]-1-piperazinyl}acetamide
N-{2-[(S)-2,6-dioxo-3-piperidyl]-1-oxo-5-isoindolinyl}{4-[2-chloro-4-({m-[2-(2,2-difluoroethylamino)ethoxy]phenyl}-N-(Methyl-d3)amino)phenyl]-1-piperazinyl}acetamide
N-{2-[(S)-2,6-dioxo-3-piperidyl]-1-oxo-5-isoindolinyl}[3-(2-chloro-4-{m-[2-(2,2-difluoroethylamino)ethoxy]phenylamino}phenyl)-1-azetidinyl]acetamide
N-{2-[(S)-2,6-dioxo-3-piperidyl]-1-oxo-5-isoindolinyl}[3-(4-{m-[2-(2,2-difluoroethylamino)ethoxy]phenylamino}-2-(trifluoromethyl)phenyl)-1-azetidinyl]acetamide
3-[5-(2-{4-[2-chloro-4-({m-[2-(2,2-difluoroethylamino)ethoxy]phenyl}-N-(Methyl-d3)amino)phenyl]-1-piperazinyl}ethoxy)-1-oxo-2-isoindolinyl]-2,6-piperidinedione
3-(5-{2-[3-(2-chloro-4-{m-[2-(2,2-difluoroethylamino)ethoxy]phenylamino}phenyl)-1-azetidinyl]ethoxy}-1-oxo-2-isoindolinyl)-2,6-piperidinedione
3-(5-{2-[3-(4-{m-[2-(2,2-difluoroethylamino)ethoxy]phenylamino}-2-(trifluoromethyl)phenyl)-1-azetidinyl]ethoxy}-1-oxo-2-isoindolinyl)-2,6-piperidinedione
N-{2-[(S)-2,6-dioxo-3-piperidyl]-1-oxo-5-isoindolinyl}(4-{2-chloro-4-[p-(1-piperazinyl)phenylamino]phenyl}-1-piperazinyl)acetamide
N-{2-[(S)-2,6-dioxo-3-piperidyl]-1-oxo-5-isoindolinyl}(3-{2-chloro-4-[p-(1-piperazinyl)phenylamino]phenyl}-1-azetidinyl)acetamide
N-{2-[(S)-2,6-dioxo-3-piperidyl]-1-oxo-5-isoindolinyl}(3-{4-[p-(1-piperazinyl)phenylamino]-2-(trifluoromethyl)phenyl}-1-azetidinyl)acetamide
3-{5-[2-(4-{2-chloro-4-[p-(1-piperazinyl)phenylamino]phenyl}-1-piperazinyl)ethoxy]-1-oxo-2-isoindolinyl}-2,6-piperidinedione
3-{1-oxo-5-[2-(3-{4-[p-(1-piperazinyl)phenylamino]-2-(trifluoromethyl)phenyl}-1-azetidinyl)ethoxy]-2-isoindolinyl}-2,6-piperidinedione
N-{2-[(S)-2,6-dioxo-3-piperidyl]-1-oxo-5-isoindolinyl}{3-[2-chloro-4-({m-[2-(2-fluoroethylamino)ethoxy]phenyl}-N-methylamino)phenyl]-1-azetidinyl}acetamide
{2-[p-({3-chloro-4-[4-({N-2-[(S)-2,6-dioxo-3-piperidyl]-1-oxo-5-isoindolinylcarbamoyl}methyl)-1-piperazinyl]phenyl}-N-methylamino)phenoxy]ethylamino}acetamide
N-{2-[(S)-2,6-dioxo-3-piperidyl]-1-oxo-5-isoindolinyl}{4-[2-chloro-4-({p-[2-(2-fluoroethylamino)ethoxy]phenyl}-N-(Methyl-d3)amino)phenyl]-1-piperazinyl}acetamide
N-{2-[(S)-2,6-dioxo-3-piperidyl]-1-oxo-5-isoindolinyl}[3-(4-{[p-(2-hydroxyethoxy)phenyl]-N-methylamino}-2-(trifluoromethyl)phenyl)-1-azetidinyl]acetamide
(S)-3-(5-{2-[4-(2-chloro-4-{[5-(2-hydroxyethoxy)-2-pyrimidinyl]-N-(Methyl-d3)amino}phenyl)-1-piperazinyl]ethoxy}-1-oxo-2-isoindolinyl)-2,6-piperidinedione
N-{2-[(S)-2,6-dioxo-3-piperidyl]-1-oxo-5-isoindolinyl}(4-{2-chloro-4-[(5-{2-[(cyanomethyl)amino]ethoxy}-2-pyrimidinyl)-N-(Methyl-d3)amino]phenyl}-1-piperazinyl)acetamide
[2-(2-{[3-chloro-4-(4-{2-[2-(2,6-dioxo-3-piperidyl)-1-oxo-5-isoindolinyloxy]ethyl}-1-piperazinyl)phenyl]-N-(Methyl-d3)amino}-5-pyrimidinyloxy)ethylamino]acetonitrile
N-{2-[(S)-2,6-dioxo-3-piperidyl]-1-oxo-5-isoindolinyl}{3-[4-(m-{2-[(cyanomethyl)amino]ethoxy}phenylamino)-2-(trifluoromethyl)phenyl]-1-azetidinyl}acetamide

### EXAMPLES

The present invention is further exemplified by the following examples. The examples are for illustrative purpose(s) only and are not intended, nor should they be construed as limiting the invention in any manner. Those skilled in the art will appreciate that variations and modifications can be made without changing the scope of the invention.

### Example 1

### Preparation of Compounds

Nuclear magnetic resonance (NMR) and mass spectrometry (MS) spectra obtained for compounds described in the examples below and those described herein were consistent with that of the compounds of formulae herein.

### Liquid Chromatography-Mass Spectrometry (LC-MS) Method:

1.1 Samples are run on Agilent Technologies 6120 MSD system with a Zorbax Eclipse XDB-C18 (3.5 m) reverse phase column (4.6×50 mm) run at room temperature with flow rate of 1.5 mL/minute.
1.2 Samples are run on Agilent Technologies 6120 MSD system with a Symmetry-C18 (5 um) reverse phase column (4.6×50 mm) run at room temperature with flow rate of 2.0 mL/minute.
1.3 Samples are run on Agilent Technologies 6120 MSD system with a Symmetry-C18 (5 um) reverse phase column (4.6×50 mm) run at room temperature with flow rate of 2.0 mL/minute.
2.1 The mobile phase uses solvent A (water/0.1% formic acid) and solvent B (acetonitrile/0.1% formic acid): 95%/5% to 0%/100% (A/B) for 5 minutes.
2.2 The mobile phase uses solvent A (water/0.1% trifluoroacetic acid) and solvent B (Acetonitrile): 90%/10% to 5%/95% (A/B) for 3.5 minutes.
2.3 The mobile phase uses solvent A (water/0.05% formic acid) and solvent B (Acetonitrile/0.05% formic acid): 90%/10% to 5%/95% (A/B) for 3.5 minutes.
3.1 The mass spectra (m/z) were recorded using electrospray ionization (ESI).
3.2 Ionization data was rounded to the nearest integer.

### Proton NMR Spectra:

Unless otherwise indicated, all ¹H NMR spectra are run on a Varian series Mercury 400 or 500 MHz. All observed protons are reported as parts-per-million (ppm) downfield from tetramethylsilane using conventional abbreviations for designation of major peaks: e.g., s (singlet), d (doublet), t (triplet), q (quartet), m (multiplet), and brs (broad singlet).

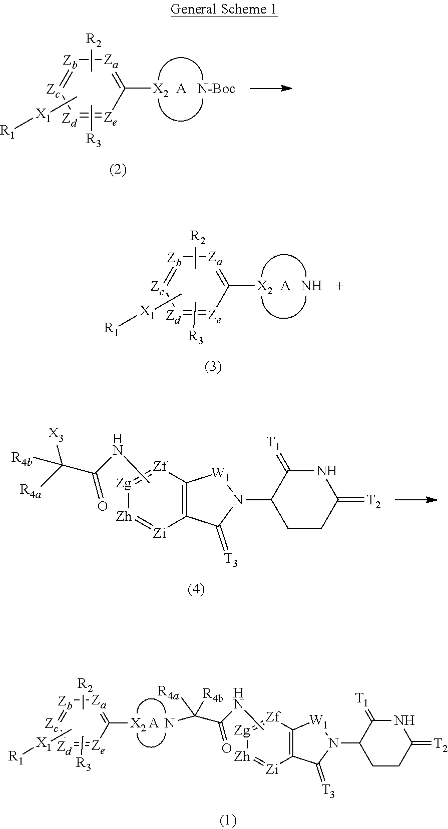

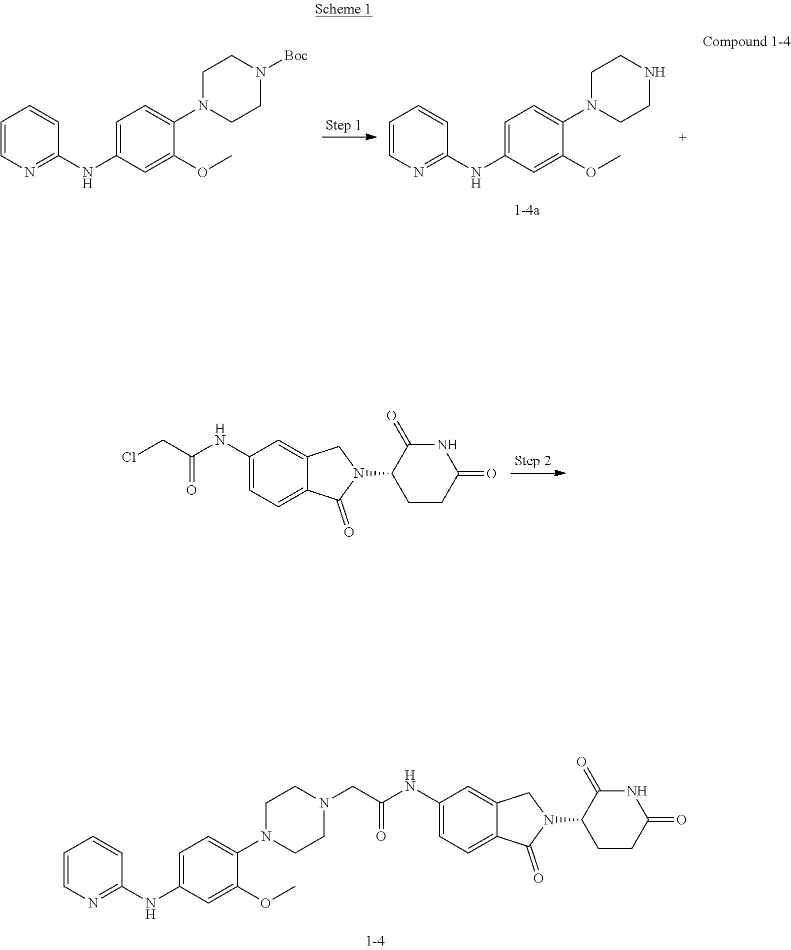 [SMILES: COc1cc(Nc2ccccn2)ccc1N1CCN(C(=O)OC(C)(C)C)CC1 ; COc1cc(Nc2ccccn2)ccc1N1CCNCC1 ; O=C1CC[C@H](N2Cc3cc(NC(=O)CCl)ccc3C2=O)C(=O)N1 ; COc1cc(Nc2ccccn2)ccc1N1CCN(CC(=O)Nc2ccc3c(c2)CN([C@H]2CCC(=O)NC2=O)C3=O)CC1]

Step 1: To a solution of 4-[2-methoxy-4-(2-pyridylamino)phenyl]piperazine-1-carboxylate (390 mg, 4.12 mmol) in DCM (5 mL) was added TFA (2 mL) dropwise at 0° C. The reaction mixture was stirred at room temperature for 2 hours. The reaction mixture was concentrated to afford a crude product as a yellow solid. The crude product was washed with MTBE (200 mL) and then filtered. The filter cake was concentrated in vacuum to give 1-4a (400 mg, 98% yield) as a yellow solid. ESI-MS: m/z=285.2 [M+1]⁺.
Step 2: A solution of 1-4a (114 mg, 286.16 μmol), (S)-2-chloro-N-(2-(2,6-dioxopiperidin-3-yl)-1-oxoisoindolin-5-yl)acetamide (96.08 mg, 286.16 μmol) and TEA (144.78 mg, 1.43 mmol) in DMF (2 mL) was stirred at 90° C. for 16 hours. The reaction mixture was then concentrated in vacuo and purified by silica gel chromatography (DCM/MeOH=30:1 to 20:1) to obtain compound 1-4 (40.2 mg, 24% yield). ESI-MS: m/z=584.4 [M+1]⁺.

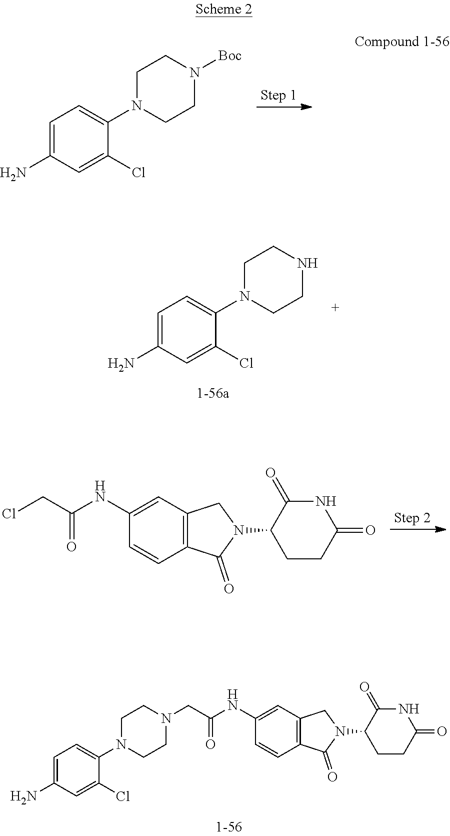 [SMILES: CC(C)(C)OC(=O)N1CCN(c2ccc(N)cc2Cl)CC1 ; Nc1ccc(N2CCNCC2)c(Cl)c1 ; O=C1CC[C@H](N2Cc3cc(NC(=O)CCl)ccc3C2=O)C(=O)N1 ; Nc1ccc(N2CCN(CC(=O)Nc3ccc4c(c3)CN([C@H]3CCC(=O)NC3=O)C4=O)CC2)c(Cl)c1]

Step 1: A solution of tert-butyl 4-(4-amino-2-chlorophenyl)piperazine-1-carboxylate (286.3 mg, 0.92 mmol) and 4N HCl/dioxane (5 mL) in DCM (8 mL) was stirred at 25° C. for 16 hours. The mixture was concentrated to afford a crude product. The solid was stirred in MTBE (10 mL) for 6 hours. The mixture was filtered, and the filter cake was dried to dryness to give 1-56a (82.7 mg, 42.6% yield) as a gray solid. ESI-MS: m/z=212.1 [M+1]⁺.
Step 2: A solution of 1-56a (82.72 mg, 0.39 mmol), (S)-2-chloro-N-(2-(2,6-dioxopiperidin-3-yl)-1-oxoisoindolin-5-yl)acetamide (130.7 mg, 0.39 mmol), and DIEA (150.9 mg, 1.17 mmol) in DMSO (5 mL) was stirred at 80° C. for 12 hours. The reaction mixture was poured into water (10 mL) and extracted with EtOAc (10 mL×3). The combined organic layers were washed with brine, dried over Na₂SO₄, and concentrated to afford a crude product. The residue was purified by prep-HPLC and lyophilized to give 1-56 (20.0 mg, 10.1% yield) as a white solid. ¹H NMR (400 MHz, DMSO_d₆): δ 10.98 (s, 1H), 10.11 (s, 1H), 8.14 (s, 0H), 8.02 (s, 1H), 7.68 (q, J=8.2 Hz, 2H), 6.91 (d, J=8.4 Hz, 1H), 6.63 (d, J=2.0 Hz, 1H), 6.49 (dd, J=8.4, 2.0 Hz, 1H), 5.09 (dd, J=13.2, 4.8 Hz, 1H), 4.43 (d, J=17.4 Hz, 1H), 4.30 (d, J=17.2 Hz, 1H), 3.26 (s, 2H), 2.88 (s, 4H), 2.68 (s, 4H), 2.64-2.51 (m, 2H), 2.38 (dt, J=13.2, 9.5 Hz, 1H), 2.06-1.93 (m, 1H). ESI-MS: m/z=511.2 [M+1]⁺.

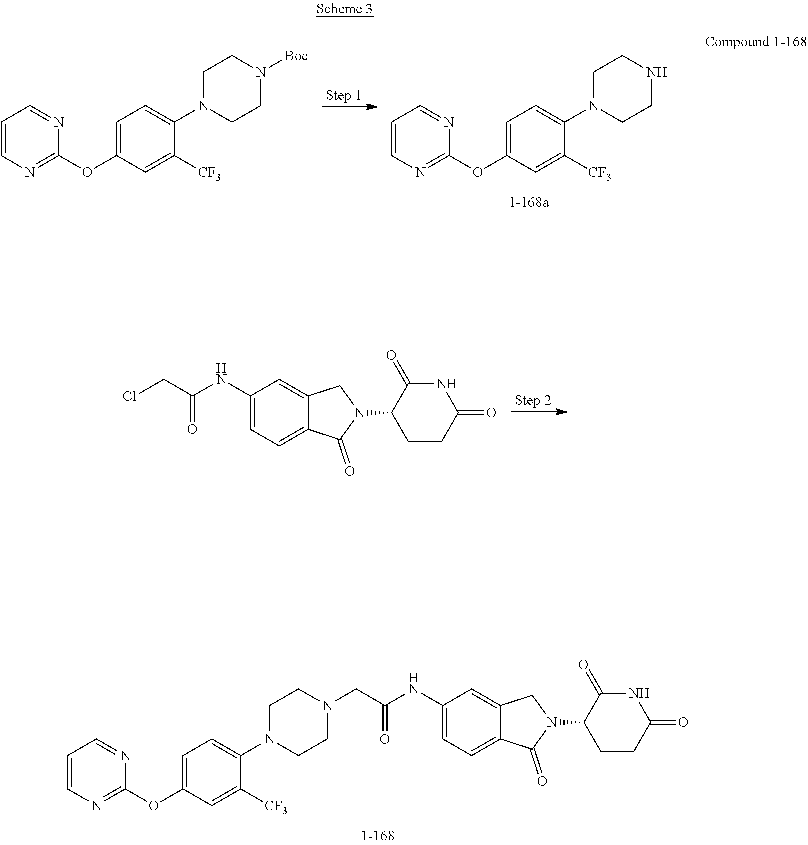 [SMILES: CC(C)(C)OC(=O)N1CCN(c2ccc(Oc3ncccn3)cc2C(F)(F)F)CC1 ; FC(F)(F)c1cc(Oc2ncccn2)ccc1N1CCNCC1 ; O=C1CC[C@H](N2Cc3cc(NC(=O)CCl)ccc3C2=O)C(=O)N1 ; O=C1CC[C@H](N2Cc3cc(NC(=O)CN4CCN(c5ccc(Oc6ncccn6)cc5C(F)(F)F)CC4)ccc3C2=O)C(=O)N1]

Step 1: To a solution of tert-butyl 3-(4-(pyrimidin-2-yloxy)-2-(trifluoromethyl) phenyl) azetidine-1-carboxylate (440 mg, 1.04 mmol) in DCM (5 mL) was added TFA (2.36 g, 20.73 mmol) at 0° C. The reaction mixture was stirred at room temperature for 2 hours. The reaction mixture was then concentrated in vacuo and purified by silica gel chromatography (DCM/MeOH=10:1) to obtain 1-168a (380 mg, quant.). ESI-MS: m/z=325.3 [M+1]⁺.
Step 2: A solution of 1-168a (60 mg, 136.89 μmol), (S)-2-chloro-N-(2-(2,6-dioxopiperidin-3-yl)-1-oxoisoindolin-5-yl)acetamide (45.96 mg, 136.89 μmol), and TEA (69.26 mg, 684.43 μmol) in DMF (2 mL) was stirred at 110° C. for 4 hours. The reaction mixture was then concentrated in vacuo and purified by silica gel chromatography (DCM/MeOH=30:1 to 20:1) to obtain 1-168 (51.6 mg, 60% yield). ESI-MS: m/z=624.5 [M+1]⁺.

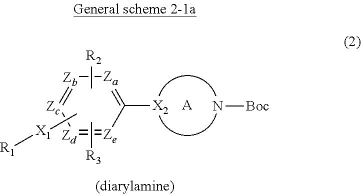

### Synthesis of Diarylamine Intermediates

### Method 1

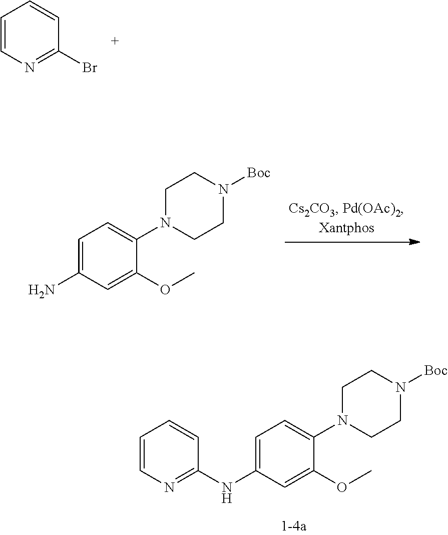 [SMILES: Brc1ccccn1 ; COc1cc(N)ccc1N1CCN(C(=O)OC(C)(C)C)CC1 ; COc1cc(Nc2ccccn2)ccc1N1CCN(C(=O)OC(C)(C)C)CC1]

To a solution of 2-bromopyridine (300 mg, 1.90 mmol) in 1,4-dioxane (10 mL) under nitrogen was added tert-butyl 4-(4-amino-2-methoxyphenyl)piperazine-1-carboxylate (525.3 mg, 1.71 mmol), Cs₂CO₃ (1.24 g, 3.80 mmol), Pd(OAc)₂ (42.63 mg, 189.88 μmol), and Xantphos (109.87 mg, 189.88 μmol) at 20° C. The reaction mixture was stirred at 100° C. for 18 hours. The reaction mixture was poured into water (30 mL) and extracted with EtOAc (30 mL×3). The combined organic layers were washed with water (30 mL) and brine (30 mL), dried over Na₂SO₄, and concentrated to afford a crude product. The crude product was purified by silica gel chromatography (DCM:MeOH=30:1) to give tert-butyl 4-[2-methoxy-4-(2-pyridylamino)-phenyl]piperazine-1-carboxylate (1-4a, 390 mg, 53% yield) as a light brown solid. ESI-MS: m/z=385.2 [M+1]⁺.

### Method 2

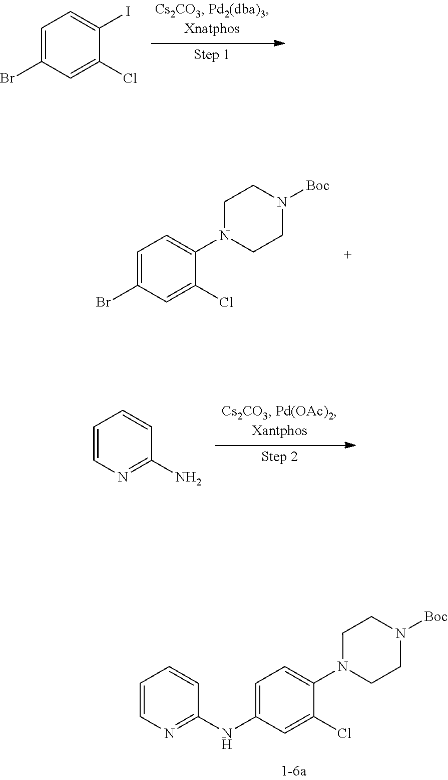 [SMILES: Clc1cc(Br)ccc1I ; CC(C)(C)OC(=O)N1CCN(c2ccc(Br)cc2Cl)CC1 ; Nc1ccccn1 ; CC(C)(C)OC(=O)N1CCN(c2ccc(Nc3ccccn3)cc2Cl)CC1]

Step 1: To a solution of 4-bromo-2-chloro-1-iodobenzene (10.00 g, 31.5 mmol) in 1,4-dioxane (100 mL) was added tert-butyl piperazine-1-carboxylate (5.9 g, 31.5 mmol), Cs₂CO₃ (30.79 g, 94.5 mmol), Pd₂(dba)₃ (2 g), and Xantphos (2 g). The reaction mixture was stirred at 110° C. for 16 hours under N₂. The mixture was filtered, and the filter cake was washed with EtOAc. The filtrate was poured into water (200 mL) and extracted with EtOAc (200 mL×3). The combined organic layers were washed with brine, dried over Na₂SO₄, and concentrated to afford a crude product. The crude product was purified by silica gel chromatography (PE/EtOAc=100/1˜10/1) to give tert-butyl 4-(4-bromo-2-chlorophenyl)piperazine-1-carboxylate (7.00 g, 36.0% yield) as a yellow solid. ESI-MS: m/z=377.0, 375.0 [M+1]⁺, 319.0, 321.0 [M+1−56]⁺.
Step 2: To a solution of pyridine-2-amine (325.67 mg, 3.46 mmol) in 1,4-dioxane (10 mL) under nitrogen was added tert-butyl 4-(4-bromo-2-chlorophenyl)piperazine-1-carboxylate (1.0 g, 2.66 mmol), Cs₂CO₃ (2.17 g, 6.65 mmol), Pd(OAc)₂ (41.83 mg, 186.32 μmol), and Xantphos (107.81 mg, 186.32 μmol) at 20° C. The reaction mixture was stirred at 100° C. for 18 hours. The reaction mixture was poured into water (30 mL) and extracted with EtOAc (30 mL×3). The combined organic layers were washed with water (30 mL) and brine (30 mL), dried over Na₂SO₄, and concentrated to give tert-butyl 4-(2-chloro-4-(pyridin-2-ylamino)phenyl)piperazine-1-carboxylate (1-6a, 0.98 g, 94% yield).

### Method 3

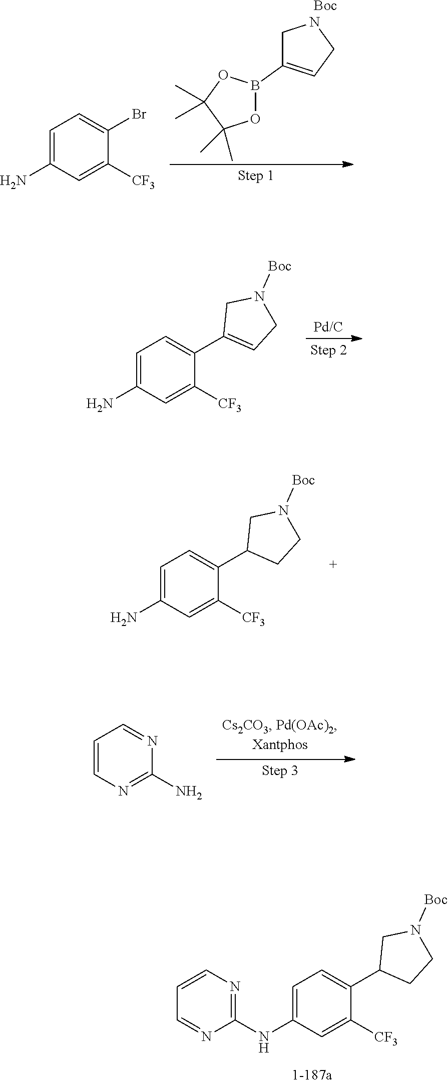

Step 1: To a solution of 4-bromo-3-(trifluoromethyl) aniline (5.00 g, 20.8 mmol) in 1,4 dioxane:H₂O=5:1 (60 mL) was added tert-butyl 3-(4,4,5,5-tetramethyl-1,3,2-dioxaborolan-2-yl)-2,5-dihydro-1H-pyrrole-1-carboxylate (6.16 g, 20.8 mmol), K₂CO₃ (5.75 g, 41.6 mmol), and Tetrakis(triphenylphosphine)Palladium (1.00 g). The reaction was stirred at 80° C. for 16 hours. The reaction mixture was poured into ice-water (300 mL) and extracted with EtOAc (200 mL×3). The combined organic layers were washed with brine, dried over Na₂SO₄, and concentrated to afford a crude product. The crude product was purified by silica gel chromatography (PE/EtOAc=50/1˜20/1) to give tert-butyl 3-(4-amino-2-(trifluoromethyl) phenyl)-2,5-dihydro-1H-pyrrole-1-carboxylate (3.00 g, 40.8% yield) as a yellow solid. ESI-MS: m/z=329.0[M+1]⁺, 273.0[M+1−56]⁺
Step 2: A mixture of tert-butyl 3-(4-amino-2-(trifluoromethyl) phenyl)-2,5-dihydro-1H-pyrrole-1-carboxylate (3.00 g, 9.1 mmol) and 10% Pd/C (1.45 g, 50% wet) in MeOH (30 mL) was stirred at 30° C. for 16 hours under 20 psi of H₂. The mixture was filtered, and the filtrate was concentrated to give the crude product. The crude product was purified by prep-TLC (PE/EtOAc=5/1) to give tert-butyl 3-(4-amino-2-(trifluoromethyl) phenyl) pyrrolidine-1-carboxylate (0.50 g, 15.9% yield) as a white solid. ¹H NMR (400 MHz, DMSO_d₆) δ 7.27 (t, J=7.2 Hz, 1H), 6.86 (d, J=2.4 Hz, 1H), 6.78 (dd, J=8.4, 2.0 Hz, 1H), 5.46 (s, 2H), 3.56-3.51 (m, 1H), 3.49-3.36 (m, 2H), 3.30-3.22 (m, 1H), 3.14-3.06 (m, 1H), 2.06-1.88 (m, 2H), 1.40 (d, J=9.2 Hz, 9H). ESI-MS: m/z=275.0 [M+1−56]⁺
Step 3: To a solution of 2-chloropyrimidine (397.96 mg, 3.47 mmol) in 1,4-dioxane (10 mL) under nitrogen was added tert-butyl 3-(4-amino-2-(trifluoromethyl) phenyl) pyrrolidine-1-carboxylate (1.0 g, 3.02 mmol), Cs₂CO₃ (1.96 g, 5.79 mmol), Pd(OAc)₂ (67.80 mg, 302 μmol), and Xantphos (174.74 mg, 302 μmol) at 20° C. The reaction mixture was stirred at 100° C. for 18 hours. The reaction mixture was poured into water (30 mL) and extracted with EtOAc (30 mL×3). The combined organic layers were washed with water (30 mL) and brine (30 mL), dried over Na₂SO₄, and concentrated to afford a crude product. The crude product was purified by silica gel chromatography (EtOAc/Hexane=1:3) to give tert-butyl 4-(4-(pyrimidin-2-ylamino)-2-(trifluoromethyl)phenyl)piperazine-1-carboxylate (1.0 g, 70.5% yield). ESI-MS: m/z=409.4 [M+1]⁺.

### Method 4

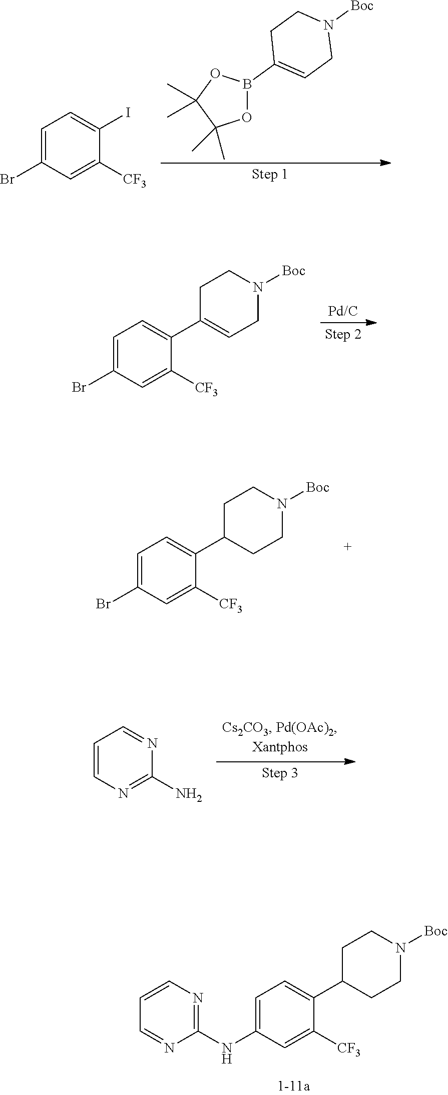

Step 1: To a solution of 4-bromo-1-iodo-2-(trifluoromethyl)benzene (10.00 g, 28.50 mmol) in 1,4-dioxane/H₂O (5/1,120 mL) was added tert-butyl 4-(4,4,5,5-tetramethyl-1,3,2-dioxaborolan-2-yl)-3,6-dihydropyridine-1(2H)-carboxylate (8.90 g, 28.50 mmol), Pd(PPh₃)₄ (1.65 g, 1.40 mmol), and K₂CO₃ (11.82 g, 85.50 mmol). The reaction mixture was stirred at 80° C. for 16 hours under N₂. The reaction mixture was poured into water (500 mL) and extracted with EtOAc (150 mL×3). The combined organic layers were washed with brine, dried over Na₂SO₄, and concentrated to afford a crude product. The crude product was purified by silica gel chromatography (PE/EtOAc=20/1˜5/1) to give tert-butyl 4-(4-bromo-2-(trifluoromethyl)phenyl)-3,6-dihydropyridine-1(2H)-carboxylate (3.50 g, 30.2% yield) as a yellow oil. ESI-MS: m/z=349.9, 351.9 [M+1]⁺.
Step 2: A solution of tert-butyl 4-(4-bromo-2-(trifluoromethyl)phenyl)-3,6-dihydropyridine-1(2H)-carboxylate (3.50 g, 8.61 mmol) and 10% Pd/C (350 mg) in EtOH (100 mL) was stirred under H₂ balloon at 25° C. for 5 hours. The mixture was filtered, and the filtrate was concentrated to afford a crude product. The crude product was purified by silica gel chromatography (PE/ETOAC=20/1˜5/1) to give tert-butyl 4-(4-bromo-2-(trifluoromethyl)phenyl)piperidine-1-carboxylate (2.55 g, 72% yield). ESI-MS: m/z=408.3, 410.3 [M+1]⁺.
Step 3: To a solution of 2-aminopyrimidine (142.66 mg, 1.50 mmol) in 1,4-dioxane (10 mL) under nitrogen was added tert-butyl 4-(4-bromo-2-(trifluoromethyl)phenyl)piperidine-1-carboxylate (592 mg, 1.45 mmol), Cs₂CO₃ (943.42 mg, 2.90 mmol), Pd(OAc)₂ (32.50 mg, 144.78 μmol), and Xantphos (83.77 mg, 144.78 μmol) at 20° C. The reaction mixture was stirred at 100° C. for 18 hours. The reaction mixture was poured into water (30 mL) and extracted with EtOAc (30 mL×3). The combined organic layers were washed with water (30 mL) and brine (30 mL), dried over Na₂SO₄, and concentrated to afford a crude product. The crude product was purified by silica gel chromatography (EtOAc/Hexane=1:15) to give tert-butyl 4-(4-(pyrimidin-2-ylamino)-2-(trifluoromethyl)phenyl)piperidine-1-carboxylate (439 mg, 71% yield). ESI-MS: m/z=423.4 [M+1]⁺.

### Method 5

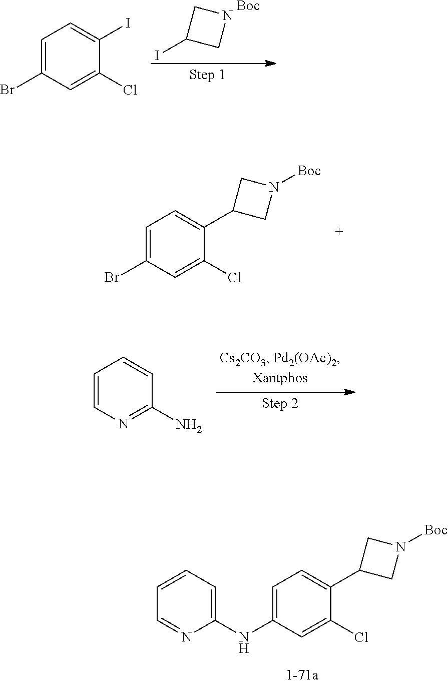

Step 1: To a stirred solution of Zn (9.0 g, 0.14 mol) in THF (150 mL) was added 1,2-Dibromoethane (3.17 g, 16.8 mmol). The reaction mixture was stirred at 80° C. for 10 minutes. Then the reaction mixture was cooled to room temperature, and chlorotrimethylsilane (1.72 g, 15.8 mmol) in THF (60 mL) was slowly added dropwise. The reaction was stirred at 25° C. for 4 minutes. Then tert-butyl 3-iodoazetidine-1-carboxylate (30.0 g, 0.11 mol) in THF (150 mL) was slowly added dropwise. The reaction was stirred at 25° C. for 2 hours. Then 4-bromo-2-chloro-1-iodobenzene (40.2 g, 0.13 mol) in THF (540 mL) was slowly added dropwise followed by tris (dibenzylideneacetone) dipalladium (1.55 g, 1.60 mmol) and Tri (2-furyl) phosphine (1.4 g, 6.10 mmol). The reaction was stirred at 55° C. for 3 hours. The reaction mixture was poured into water (1 L) and extracted with EtOAc (1 L×3). The combined organic layers were washed with brine, dried over Na₂SO₄, and concentrated to afford a crude product. The crude product was purified by silica gel chromatography (PE/EtOAc=100/1˜20/1) to give the tert-butyl 3-(4-bromo-2-chlorophenyl) azetidine-1-carboxylate (17.5 g, 39.0% yield) as a white solid. ¹H NMR (400 MHz, DMSO_d₆) δ 7.72 (d, J=1.2 Hz, 1H), 7.57 (d, J=8.0 Hz, 1H), 7.49 (d, J=8.4 Hz, 1H), 4.23 (d, J=6.8 Hz, 2H), 4.02 (m, 1H), 3.95-3.86 (m, 2H), 1.38 (s, 9H). ESI-MS: m/z=289.9 [M+1−56]⁺.
Step 2: To a solution of 2-aminopyridine (59.41 mg, 631.25 μmol) in 1,4-dioxane (10 mL) under nitrogen was added tert-butyl 3-(4-bromo-2-chlorophenyl)azetidine-1-carboxylate (182.35 mg, 526.04 μmol), Cs₂CO₃ (342.79 mg, 1.05 mmol), Pd(OAc)₂ (11.81 mg, 52.60 μmol), and Xantphos (30.44 mg, 52.60 μmol) at 20° C. The reaction mixture was stirred at 120° C. for 18 hours. The reaction mixture was filtered, concentrated, and purified by silica gel chromatography (EtOAc/Hexane=1:15) to give tert-butyl 3-(2-chloro-4-(pyridin-2-ylamino)phenyl)azetidine-1-carboxylate (1-71a, 130 mg, 68% yield). ESI-MS: m/z=360.8 [M+1]⁺.

### Method 6

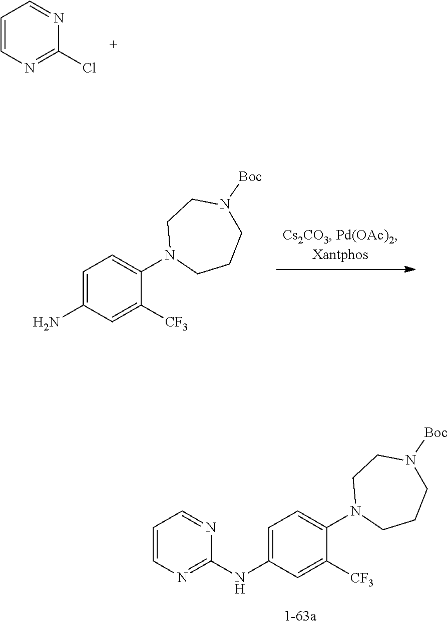 [SMILES: Clc1ncccn1 ; CC(C)(C)OC(=O)N1CCCN(c2ccc(N)cc2C(F)(F)F)CC1 ; CC(C)(C)OC(=O)N1CCCN(c2ccc(Nc3ncccn3)cc2C(F)(F)F)CC1]

To a solution of 2-chloropyrimidine (89.87 mg, 784.67 μmol) in 1,4-dioxane (3 mL) under nitrogen was added tert-butyl 4-(4-amino-2-(trifluoromethyl)phenyl)-1,4-diazepane-1-carboxylate (188 mg, 523.12 μmol), Cs₂CO₃ (340.88 mg, 1.05 mmol), Pd(OAc)₂ (11.74 mg, 52.31 μmol), and Xantphos (30.27 mg, 52.31 μmol) at 20° C. The reaction mixture was stirred at 100° C. for 18 hours. The reaction mixture was filtered, concentrated, and purified by silica gel chromatography (EtOAc/Hexane=1:10) to give tert-butyl 4-(4-(pyrimidin-2-ylamino)-2-(trifluoromethyl)phenyl)-1,4-diazepane-1-carboxylate (1-63a, 130 mg, 56% yield) as a light brown solid. ESI-MS: m/z=438.4 [M+1]⁺.

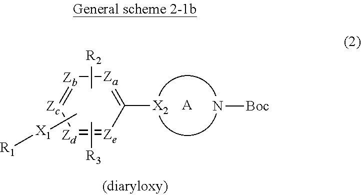

### Synthesis of Diaryl Ether Intermediates

### Method 7-1

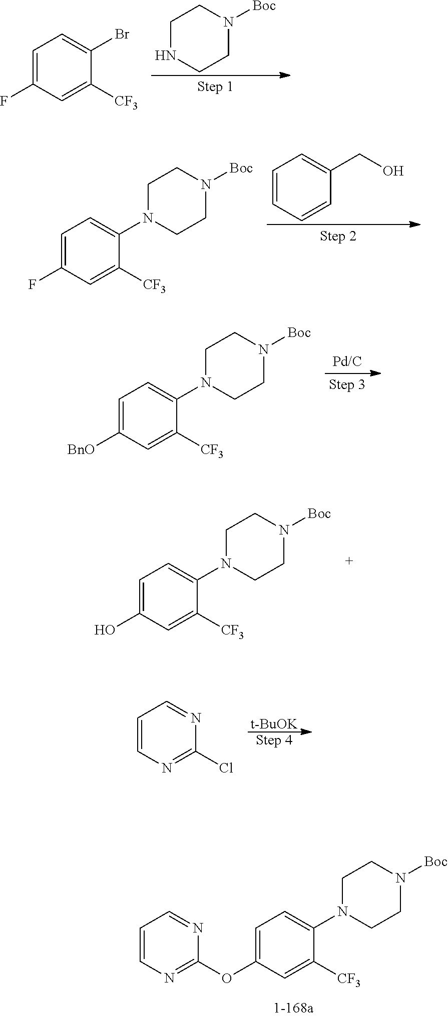

Step 1: To a solution of 1-bromo-4-fluoro-2-(trifluoromethyl)benzene (5.00 g, 20.60 mmol) in toluene (50 mL) was added tert-butyl piperazine-1-carboxylate (4.60 g, 24.70 mmol), tris(dibenzylideneacetone)dipalladium (0.94 g, 1.00 mmol), 2,2′-bis(diphenylphosphino)-1,1′-binaphthyl (0.64 g, 1.00 mmol), and Cs₂CO₃ (20.14 g, 61.80 mmol). The reaction mixture was stirred at 100° C. for 14 hours under N₂. The reaction mixture was poured into water (250 mL) and extracted with EtOAc (20 mL×3). The combined organic layers were washed with brine, dried over Na₂SO₄, and concentrated to afford a crude product. The crude product was purified by silica gel chromatography (PE/EtOAc=100/1˜20/1) to give tert-butyl 4-(4-fluoro-2-(trifluoromethyl)-phenyl)piperazine-1-carboxylate (2.30 g, 32.1% yield) as a yellow solid. ESI-MS: m/z=293.1 [M−56+1]⁺.
Step 2: A solution of tert-butyl 4-(4-fluoro-2-(trifluoromethyl)phenyl)piperazine-1-carboxylate (2.50 g, 7.20 mmol), phenylmethanol (0.93 g, 8.60 mmol) and t-BuOK (2.42 g, 21.60 mmol) in THF (30 mL) was stirred under nitrogen at 25° C. for 3 hours. The reaction mixture was poured into water (100 mL) and extracted with EtOAc (20 mL×3). The combined organic layers were washed with brine, dried over Na₂SO₄, and concentrated to afford a crude product. The crude product was purified by prep-TLC (PE/EtOAc=10/1) to give tert-butyl 4-(4-(benzyloxy)-2-(trifluoromethyl)-phenyl)piperazine-1-carboxylate (400 mg, 99% yield) as a yellow oil. ESI-MS: m/z=381.1 [M−56+1]⁺, 437.1 [M+1]⁺.
Step 3: A solution of tert-butyl 4-(4-(benzyloxy)-2-(trifluoromethyl)phenyl)piperazine-1-carboxylate (4.00 g, 9.10 mmol), 10% Pd/C (0.5 g, 50% wet) in EtOH (50 mL) was stirred at 25° C. for 3 hours under 20 psi of H₂. The mixture was filtered, and the filtrate was concentrated to afford a crude product. The crude product was purified by prep-TLC (PE/EtOAc=5/1) to give tert-butyl 4-(4-hydroxy-2-(trifluoromethyl)phenyl)piperazine-1-carboxylate (0.50 g, 15.8% yield) as an off-white solid. ESI-MS: m/z=291.1 [M−56+1]⁺, 347.3 [M+1]⁺. ¹H NMR (400 MHz, DMSO_d₆) δ 9.87 (s, 1H), 7.40 (d, J=9.2 Hz, 1H), 7.00 (d, J=6.0 Hz, 2H), 3.31 (s, 4H), 2.70 (t, J=4.4 Hz, 4H), 1.41 (s, 9H).
Step 4: A solution of tert-butyl 4-(4-hydroxy-2-(trifluoromethyl)phenyl)piperazine-1-carboxylate (1.07 g, 3.1 mmol), 2-chloropyrimidine (0.36 g, 3.1 mmol), and t-BuOK (0.52 g, 4.6 mmol) in DMSO (10 mL) was stirred under nitrogen at 25° C. for 3 hours. The reaction mixture was poured into water (50 mL) and extracted with EtOAc (10 mL×3). The combined organic layers were washed with brine, dried over Na₂SO₄, and concentrated to afford a crude product. The crude product was purified by prep-TLC (PE/EtOAc=0/1) to give tert-butyl 3-(4-(pyrimidin-2-yloxy)-2-(trifluoromethyl) phenyl) azetidine-1-carboxylate (1-168a, 0.5 g, yield 38%) as a yellow solid. ESI-MS: m/z=425.3 [M+1]⁺.

### Method 7-2

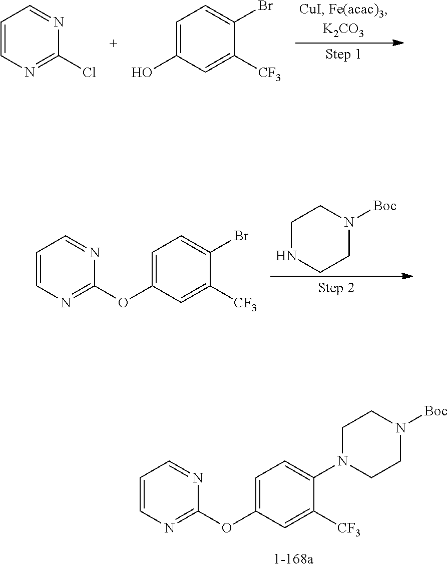 [SMILES: Clc1ncccn1 ; Oc1ccc(Br)c(C(F)(F)F)c1 ; FC(F)(F)c1cc(Oc2ncccn2)ccc1Br ; CC(C)(C)OC(=O)N1CCNCC1 ; CC(C)(C)OC(=O)N1CCN(c2ccc(Oc3ncccn3)cc2C(F)(F)F)CC1]

Step 1: A solution of 2-chloropyrimidine (712.85 mg, 6.22 mmol), 4-bromo-3-(trifluoromethyl) phenol (1.00 g, 4.15 mmol), CuI (118.53 mg, 622.39 μmol), Iron (III) acetylacetonate (443.38 mg, 1.24 mmol), and K₂CO₃ (1.43 g, 10.37 mmol) in DMF (10 mL) was stirred at 135° C. for 20 hours. The reaction mixture was then concentrated in vacuo and purified by silica gel chromatography (EtOAc/Hexane=3:7) to obtain 2-[4-bromo-3-(trifluoromethyl)-phenoxy]pyrimidine (1.18 g, 88% yield). ESI-MS: m/z=319.1, 321.1 [M+1]⁺.
Step 2: A solution of 2-[4-bromo-3-(trifluoromethyl)phenoxy]pyrimidine (1.18 g, 3.70 mmol), tert-butyl piperazine-1-carboxylate (895.42 mg, 4.81 mmol), Pd(OAc)₂ (41.51 mg, 184.91 μmol), BINAP (230.27 mg, 369.82 μmol), and Cs₂CO₃ (1.81 g, 5.55 mmol) in DMF (10 mL) was stirred at 110° C. for 18 hours. The reaction mixture was filtered through a pad of Celite and concentrated in vacuo, and the residue was purified by silica gel column chromatography (EtOAc/Hexane=1:5) to obtain tert-butyl 4-[4-pyrimidin-2-yloxy-2-(trifluoromethyl)phenyl]piperazine-1-carboxylate (1-168a, 440 mg, 28% yield). ESI-MS: m/z=425.3 [M+1]⁺.

### Method 8

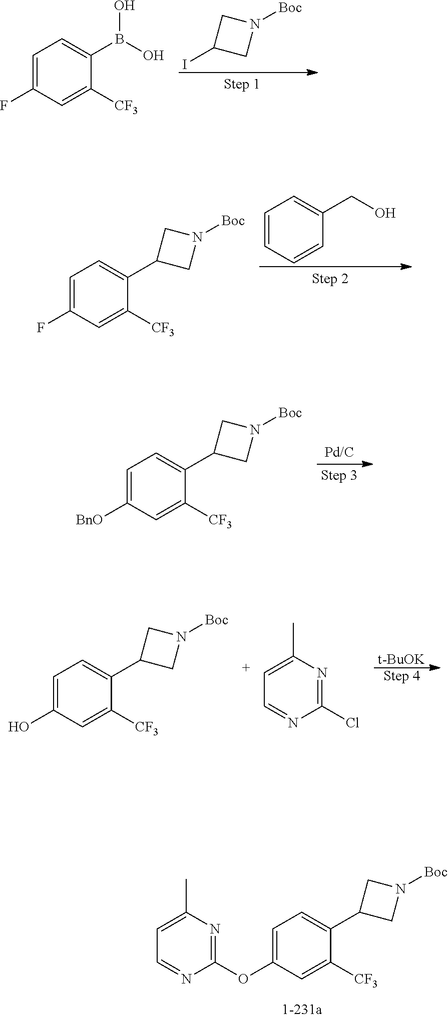

Step 1: To a solution of (4-fluoro-2-(trifluoromethyl) phenyl) boronic acid (10.00 g, 48.2 mmol) in toluene/EtOH/H₂O=2:1:1 (200 mL) was added tert-butyl 3-iodoazetidine-1-carboxylate (16.40 g, 57.7 mmol), CuCl (9.52 g, 96.2 mmol), Pd₂(dba)₃ (2 g), and S-phos (2 g). The reaction mixture was stirred at 60° C. for 16 hours under N₂. The reaction mixture was poured into water (200 mL) and extracted with EtOAc (200 mL×3). The combined organic layers were washed with brine, dried over Na₂SO₄, and concentrated to afford a crude product. The crude product was purified by silica gel chromatography (PE/EtOAc=100/1˜20/1) to give tert-butyl 3-(4-fluoro-2-(trifluoromethyl) phenyl) azetidine-1-carboxylate (3.00 g, 18.6% yield) as an off-white solid. ESI-MS: m/z=264.1 [M+1−56]⁺
Step 2: A solution of phenylmethanol (1.22 g, 11.3 mmol) and tert-butyl 3-(4-fluoro-2-(trifluoromethyl) phenyl) azetidine-1-carboxylate (3.00 g, 9.4 mmol) in DMSO (30 mL) was stirred at 0° C. Then t-BuOK (2.11 g, 18.8 mmol) was slowly added at 0° C. The reaction mixture was stirred at 25° C. for 2 hours. The reaction mixture was poured into water (150 mL) and extracted with EtOAc (200 mL×2). The combined organic layers were washed with brine, dried over Na₂SO₄, and concentrated to afford a crude product. The crude product was purified by silica gel chromatography (PE/EtOAc=100/1˜20/1) to give tert-butyl 3-(4-(benzyloxy)-2-(trifluoromethyl) phenyl) azetidine-1-carboxylate (3.00 g, 51.0% yield) as a yellow solid. ESI-MS: m/z=352.1 [M+1−56]⁺, 430.2 [M+23]⁺.
Step 3: A mixture of tert-butyl 3-(4-(benzyloxy)-2-(trifluoromethyl) phenyl) azetidine-1-carboxylate (2.00 g, 4.9 mmol) and 10% Pd/C (0.80 g, 50% wet) in MeOH (20 mL) was stirred at 20° C. for 16 hours under 20 psi of H₂. The mixture was filtered, and the filtrate was concentrated to afford a crude product. The crude product was purified by silica gel chromatography (PE/EtOAc=100/1˜5/1) to give tert-butyl 3-(4-hydroxy-2-(trifluoromethyl)phenyl)azetidine-1-carboxylate (1.33 g, 72.6%) as a white solid. ¹H NMR (400 MHz, DMSO_d₆) δ 10.05 (s, 1H), 7.63 (d, J=8.6 Hz, 1H), 7.09 (dd, J=8.8, 2.4 Hz, 1H), 7.04 (d, J=2.6 Hz, 1H), 4.19 (t, J=8.4 Hz, 2H), 4.02-3.93 (m, 1H), 3.56-3.80 (m, 2H), 1.39 (s, 9H). ESI-MS: m/z=262.1 [M+1−56]⁺
Step 4: A solution of tert-butyl 3-(4-hydroxy-2-(trifluoromethyl) phenyl) azetidine-1-carboxylate (1.00 g, 3.1 mmol), 2-chloro-4-methylpyrimidine (0.40 g, 3.1 mmol), and t-BuOK (0.52 g, 4.6 mmol) in DMSO (10 mL) was stirred under nitrogen at 25° C. for 3 hours. The reaction mixture was poured into water (50 mL) and extracted with EtOAc (10 mL×3). The combined organic layers were washed with brine, dried over Na₂SO₄, and concentrated to afford a crude product. The crude product was purified by prep-TLC (PE/EtOAc=0/1) to give tert-butyl 3-(4-((4-methylpyrimidin-2-yl) oxy)-2-(trifluoromethyl) phenyl) azetidine-1-carboxylate (1-231a, 1.00 g, yield 38.8%) as a yellow solid. ESI-MS: m/z=354.3 [M+1−56]⁺.

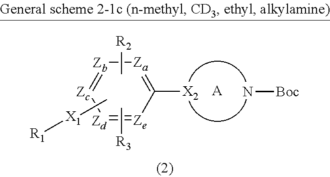

### Synthesis of Diaryl Amine Intermediate-2

### Method 9

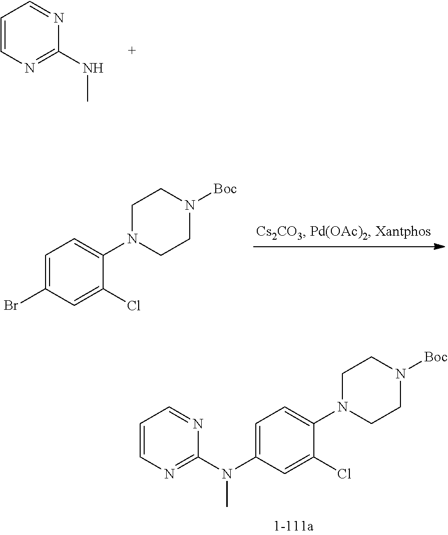

To a solution of N-methylpyrimidin-2-amine (174.29 mg, 1.60 mmol) in 1,4-dioxane (25 mL) under nitrogen was added tert-butyl 4-(4-bromo-2-chloro-phenyl)piperazine-1-carboxylate (500 mg, 1.33 mmol), Cs₂CO₃ (867.26 mg, 2.66 mmol), Pd(OAc)₂ (29.88 mg, 133.09 μmol), and Xantphos (77.01 mg, 133.09 μmol) at 20° C. The reaction mixture was stirred at 100° C. for 18 hours. The reaction mixture was poured into water (50 mL) and extracted with EtOAc (30 mL×3). The combined organic layers were washed with water (30 mL) and brine (30 mL), dried over Na₂SO₄, and concentrated to afford a crude product. The crude product was purified by silica gel chromatography (EtOAc/Hexane=1:5) to give tert-butyl 4-(2-chloro-4-(methyl(pyrimidin-2-yl)amino)-phenyl)piperazine-1-carboxylate (1-111a, 370 mg, 68% yield) as a light brown solid. ESI-MS: m/z=404.2, 406.2 [M+1]⁺.

### Method 10

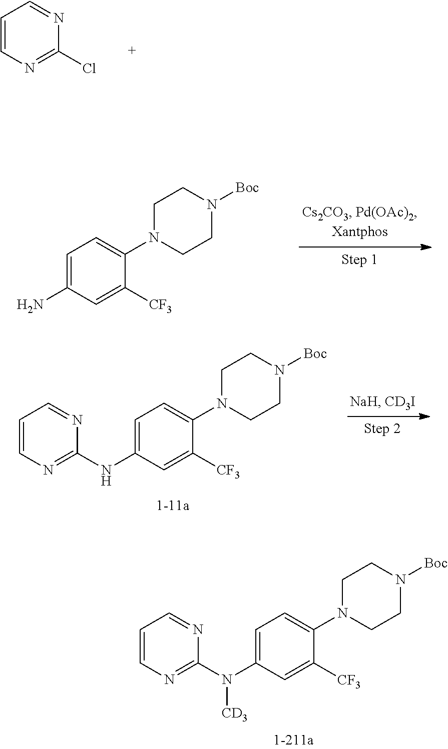 [SMILES: Clc1ncccn1 ; CC(C)(C)OC(=O)N1CCN(c2ccc(N)cc2C(F)(F)F)CC1 ; CC(C)(C)OC(=O)N1CCN(c2ccc(Nc3ncccn3)cc2C(F)(F)F)CC1 ; [2H]C([2H])([2H])N(c1ccc(N2CCN(C(=O)OC(C)(C)C)CC2)c(C(F)(F)F)c1)c1ncccn1]

Step 1: To a solution of 2-chloropyrimidine (397.96 mg, 1.50 mmol) in 1,4-dioxane (10 mL) under nitrogen was added tert-butyl 4-[4-amino-2-(trifluoromethyl)phenyl]piperazine-1-carboxylate (1.0 g, 2.90 mmol), Cs₂CO₃ (1.89 g, 5.79 mmol), Pd(OAc)₂ (65.01 mg, 289.55 μmol), and Xantphos (167.54 mg, 289.55 μmol) at 20° C. The reaction mixture was stirred at 100° C. for 18 hours. The reaction mixture was poured into water (50 mL) and extracted with EtOAc (30 mL×3). The combined organic layers were washed with water (30 mL) and brine (30 mL), dried over Na₂SO₄, and concentrated to afford a crude product. The crude product was purified by silica gel chromatography (EtOAc/Hexane=1:3) to give tert-butyl 4-(4-(pyrimidin-2-ylamino)-2-(trifluoromethyl)phenyl)piperazine-1-carboxylate (1-11a, 1.07 g, 87% yield). ESI-MS: m/z=424.3 [M+1]⁺.
Step 2: A solution of 4-(4-(pyrimidin-2-ylamino)-2-(trifluoromethyl)phenyl)piperazine-1-carboxylate (300 mg, 708.50 μmol) and NaH (34.01 mg, 1.42 mmol) in DMF (3 mL) was stirred at 0° C. for 0.5 hours. Iodomethane-d₃ (205.41 mg, 1.42 mmol) added and stirred at 25° C. for 16 hours. The reaction mixture was poured into water (5 mL) and extracted with EtOAc (30 mL×3). The combined organic layers were washed with brine, dried over Na₂SO₄, and concentrated to afford a crude product. The crude product was purified by silica gel chromatography (EtOAc/Hexane=1:5) to give tert-butyl 4-[4-[pyrimidin-2-yl(trideuteriomethyl)amino]-2-(trifluoromethyl)phenyl]piperazine-1-carboxylate (1-211a, 300 mg, 96% yield). ESI-MS: m/z=441.4 [M+1]⁺.

### Method 11

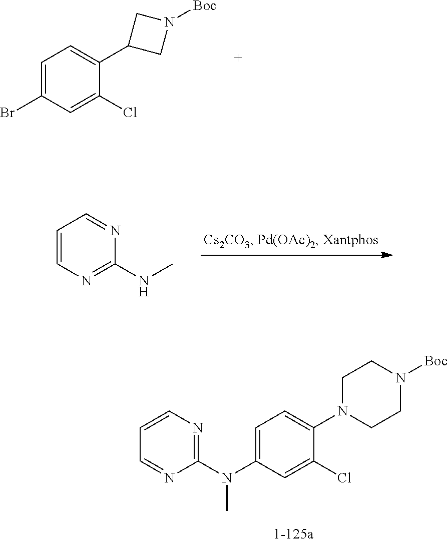

To a solution of 2-aminopyrimidine (122.78 mg, 1.13 mmol) in 1,4-dioxane (5 mL) under nitrogen was added tert-butyl 3-(4-bromo-2-chlorophenyl)azetidine-1-carboxylate (300 mg, 865.43 μmol), Cs₂CO₃ (563.95 mg, 1.73 mmol), Pd(OAc)₂ (19.43 mg, 86.54 μmol), and Xantphos (50.08 mg, 86.54 μmol) at 20° C. The reaction mixture was stirred at 100° C. for 18 hours. The reaction mixture was filtered, concentrated, and purified by silica gel chromatography (EtOAc/Hexane=1:15) to give tert-butyl 3-(2-chloro-4-(methyl(pyrimidin-2-yl)amino)phenyl)azetidine-1-carboxylate (1-125a, 240 mg, 73% yield). ESI-MS: m/z=375.8 [M+1]⁺.

### Method 12

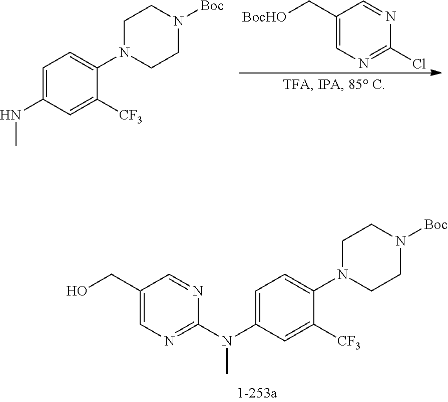

A solution of tert-butyl 4-(4-(methylamino)-2-(trifluoromethyl) phenyl) piperazine-1-carboxylate (500.0 mg, 1.39 mmol), (2-chloropyrimidin-5-yl) methanol (300.8 mg, 2.08 mmol) and TFA (0.2 mL) in IPA (5 mL) was stirred at 85° C. for 16 hours. The reaction mixture was concentrated to afford a crude product. The crude product was purified by prep-TLC (PE/EtOAc=2/1) to give tert-butyl 4-(4-((5-(hydroxymethyl) pyrimidin-2-yl) (methyl)amino)-2-(trifluoromethyl) phenyl) piperazine-1-carboxylate (1-253a, 250.0 mg, 28.83% yield) as a yellow solid.

### Method 13

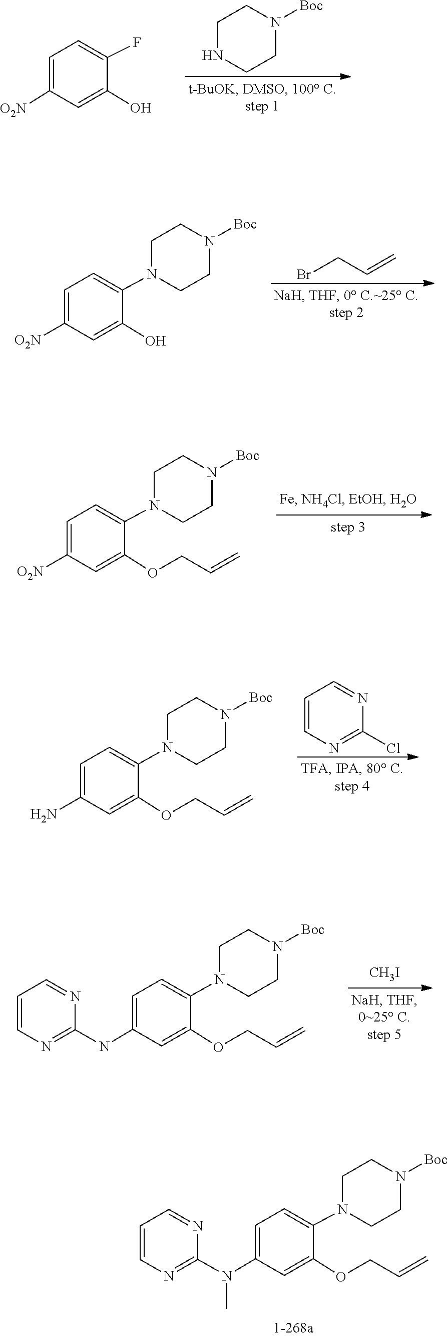

Step 1: To a solution of 2-fluoro-5-nitrophenol (10.00 g, 63.7 mmol) and tert-butyl piperazine-1-carboxylate (11.90 g, 63.7 mmol) in DMSO (100 mL) stirred under nitrogen was added t-BuOK (14.30 g, 12.7 mmol). The reaction was stirred at 100° C. for 16 hours. The reaction mixture was poured into water (500 mL) and extracted with EtOAc (200 mL×3). The combined organic layers were washed with brine, dried over Na₂SO₄ and concentrated to afford a crude product. The crude product was purified by silica gel chromatography (PE/EtOAc=100/1˜10/1) to give tert-butyl 4-(2-hydroxy-4-nitrophenyl) piperazine-1-carboxylate (4.00 g, 19.43% yield) as a yellow solid. ESI-MS: m/z=268.0 [M+1−56]⁺, 324.1 [M+1]⁺.
Step 2: To a stirred solution of tert-butyl 4-(2-hydroxy-4-nitrophenyl) piperazine-1-carboxylate (3.00 g, 9.2 mmol) in THF (30 mL) stirred under nitrogen was added NaH (0.74 g, 18.4 mmol) at 0° C. The mixture was stirred at 25° C. for 0.5 hours. Then 3-bromoprop-1-ene (1.67 g, 13.8 mmol) was slowly added dropwise. The reaction was stirred at 25° C. for 6 hours. The reaction mixture was slowly poured into ice water (100 mL) and extracted with EtOAc (100 mL×3). The combined organic layers were washed with brine, dried over Na₂SO₄ and concentrated to afford a crude product. The crude product was purified by silica gel chromatography (PE/EtOAc=100/1˜10/1) to give tert-butyl 4-(2-hydroxy-4-nitrophenyl) piperazine-1-carboxylate (1.50 g, 31.14% yield) as a yellow solid. ESI-MS: m/z=364.1 [M+1]⁺.
Step 3: To a solution of tert-butyl 4-(2-hydroxy-4-nitrophenyl) piperazine-1-carboxylate (500 mg, 1.37 mmol) in EtOH/NH₄Cl/H₂O (5 mL) stirred under nitrogen was added Fe (766 mg, 13.72 mmol). The reaction was stirred at 80° C. for 16 hours. The mixture was filtered. The filtrate was concentrated and poured into water (20 mL) and extracted with EtOAc (20 mL×3). The combined organic layers were washed with brine, dried over Na₂SO₄ and concentrated to afford a crude product. The crude product was purified by prep-TLC (PE/EtOAc=5/1) to give tert-butyl 4-(2-(allyloxy)-4-aminophenyl) piperazine-1-carboxylate (200.0 mg, 43.42% yield) as a yellow solid. ESI-MS: m/z=334.2 [M+1]⁺.
Step 4: To a solution of tert-butyl 4-(2-(allyloxy)-4-aminophenyl) piperazine-1-carboxylate (300 mg, 0.89 mmol) in IPA (4 mL) stirred was added 2-chloropyrimidine (102 mg, 0.89 mmol) and TFA (0.1 mL). The reaction was stirred at 80° C. for 16 hours. The reaction mixture was concentrated to afford a crude product. The crude product was purified by prep-TLC (PE/EtOAc=3/1) to give tert-butyl 4-(2-(allyloxy)-4-(pyrimidin-2-ylamino) phenyl) piperazine-1-carboxylate (200.0 mg, 54.02% yield) as a yellow solid. ESI-MS: m/z=412.2 [M+1]⁺.
Step 5: To a stirred solution of tert-butyl 4-(2-(allyloxy)-4-(pyrimidin-2-ylamino) phenyl) piperazine-1-carboxylate (170 mg, 0.41 mmol) in THF (5 mL) stirred under nitrogen was added NaH (33 mg, 0.82 mmol) at 0° C. The mixture was stirred at 25° C. for 0.5 hours. Then CH₃I (117 mg, 0.82 mol) was slowly added dropwise. The reaction was stirred at 25° C. for 4 hours. The reaction mixture was slowly poured into ice water (10 mL) and extracted with EtOAc (10 mL×3). The combined organic layers were washed with brine, dried over Na₂SO₄ and concentrated to afford a crude product. The crude product was purified by silica gel chromatography (PE/EtOAc=100/1˜5/1) to give tert-butyl 4-(2-(allyloxy)-4-(methyl(pyrimidin-2-yl) amino) phenyl) piperazine-1-carboxylate (1-268a, 170 mg, 87.03% yield) as a yellow oil. ESI-MS: m/z=426.2 [M+1]⁺.

### Method 14

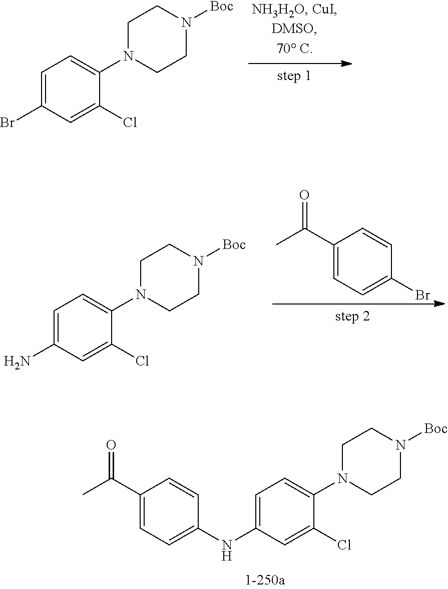

Step 1: To a solution of tert-butyl 4-(4-bromo-2-chlorophenyl) piperazine-1-carboxylate (5.00 g, 0.01 mol) in DMSO (50 mL) was added NH₃H₂O (13.32 g, 0.13 mol), Copper(I) iodide (0.13 g, 0.6 mmol), L-hydroxyproline (0.70 g, 5.0 mmol) and Potassium carbonate (5.51 g, 0.04 mol). The reaction mixture was stirred at 70° C. for 12 hours. The reaction mixture was poured into water (100 mL) and extracted with EtOAc (100 mL×3). The combined organic layers were washed with brine, dried over Na₂SO₄ and concentrated to afford a crude product. The crude product was purified by silica gel chromatography (PE/EtOAc=100/1˜5/1) to give tert-butyl 4-(4-amino-2-chlorophenyl) piperazine-1-carboxylate (2.50 g, 58.44% yield) as a yellow solid. ESI-MS: m/z=312.1 [M+1]⁺.
Step 2: To a solution of tert-butyl 4-(4-amino-2-chlorophenyl) piperazine-1-carboxylate (1.00 g, 3.2 mmol), 1-(4-bromophenyl) ethan-1-one (0.64 g, 3.2 mmol) in 1.4-dioxane (10 mL) stirred under nitrogen was added Cs₂CO₃ (3.13 g, 9.6 mmol), Pd₂(dba)₃ (0.20 g) and BINAP (0.20 g). The reaction mixture was stirred at 110° C. for 16 hours. The reaction mixture was poured into water (30 mL) and extracted with EtOAc (30 mL×3). The combined organic layers were washed with brine, dried over Na₂SO₄ and concentrated to afford a crude product. The crude product was purified by silica gel chromatography (PE/EtOAc=20/1˜1/1) to give tert-butyl 4-(4-((4-acetylphenyl) amino)-2-chlorophenyl) piperazine-1-carboxylate (1-250a, 700.0 mg, 50.77% yield) as a yellow oil. ESI-MS: m/z=430.0 [M+1]⁺.

### Method 15

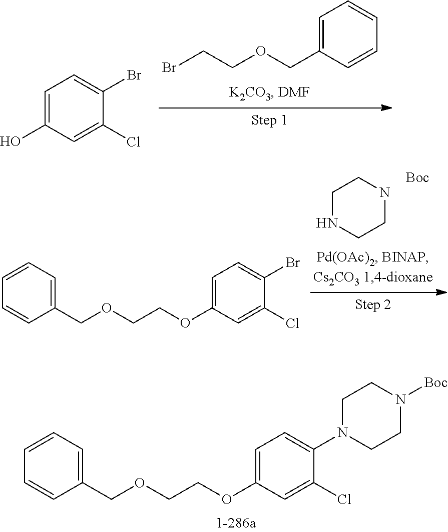

Step 1: To a solution of 4-bromo-3-chloro-phenol (500 mg, 2.41 mmol) in DMF (10 mL) was added 2-bromoethoxymethylbenzene (622.08 mg, 2.89 mmol). The reaction mixture was stirred at 30° C. for 16 hours. The mixture was concentrated in vacuum to afford a crude product. The residue was purified by silica gel column (EtOAc/Hexane=1:10) to give 4-(2-benzyloxyethoxy)-1-bromo-2-chloro-benzene (600 mg, 72.8% yield) as a solid.
Step 2: To a solution of 4-(2-benzyloxyethoxy)-1-bromo-2-chloro-benzene (600 mg, 1.76 mmol) in 1,4-dioxane (10 mL) was added Cs₂CO₃ (1.14 g, 3.51 mmol), Pd(OAc)₂ (19.72 mg, 87.82 μmol), BINAP (109.36 mg, 175.63 μmol) and tert-butyl piperazine-1-carboxylate (425.25 mg, 2.28 mmol). The reaction mixture was stirred at 110° C. for 16 hours under N₂. The reaction mixture was poured into water (300 mL) and extracted with EtOAc (50 mL×3). The combined organic layers were washed with brine, dried over Na₂SO₄ and concentrated to afford a crude product. The crude product was purified by silica gel chromatography (EtOAc/Hexane=1:10 to 1.5:8.5) to give tert-butyl 4-[4-(2-benzyloxyethoxy)-2-chloro-phenyl]piperazine-1-carboxylate (1-286a, 510 mg, 64.9% yield) as a solid. ESI-MS: m/z=447.1, 449.1 [M+1]⁺.

### Method 16

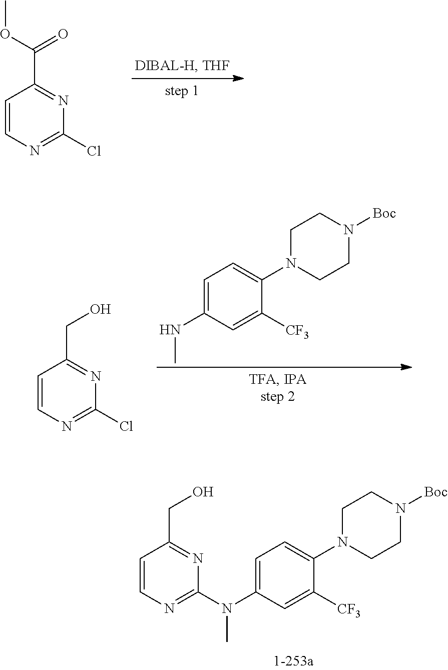 [SMILES: COC(=O)c1ccnc(Cl)n1 ; OCc1ccnc(Cl)n1 ; CNc1ccc(N2CCN(C(=O)OC(C)(C)C)CC2)c(C(F)(F)F)c1 ; CN(c1ccc(N2CCN(C(=O)OC(C)(C)C)CC2)c(C(F)(F)F)c1)c1nccc(CO)n1]

Step 1: To a stirred solution of methyl 2-chloropyrimidine-4-carboxylate (1.0 g, 5.8 mmol) in THF (10 mL) was added DIBAL-H (1 mol/L) (7.8 mL, 7.8 mmol) dropwise at 0° C. The mixture was stirred at 0° C. for 16 hours. The reaction mixture was poured into water (50 mL) and extracted with EtOAc (30 mL×3). The combined organic layers were washed with brine, dried over Na₂SO₄ and concentrated to afford a crude product. The crude product was purified by silica gel chromatography (PE/EtOAc=100/1˜10/1) to give the (2-chloropyrimidin-4-yl) methanol (0.5 g, 59.7%) as a yellow oil. ¹H NMR (400 MHz, DMSO_d₆) δ 4.56 (d, J=5.6 Hz, 2H), 5.77 (t, J=5.8 Hz, 1H), 7.59 (d, J=5.2 Hz, 1H), 8.75 (d, J=5.6 Hz, 1H).
Step 2: To a solution of (2-chloropyrimidin-4-yl) methanol (0.5 g, 3.5 mmol) in IPA (8 mL) was added tert-butyl 4-(4-(methylamino)-2-(trifluoromethyl) phenyl) piperazine-1-carboxylate (1.2 g, 3.5 mmol) and TFA (0.5 g). The reaction mixture was stirred at 80° C. for 16 hours under N₂. The reaction mixture was concentrated to give the crude product. The crude product was purified by prep-TLC (PE/EtOAc=3/1) to give the tert-butyl 4-(4-((4 (hydroxymethyl)pyrimidin-2-yl) (methyl)amino)-2 (trifluoromethyl) phenyl) piperazine-1-carboxylate (1-253a, 280 mg, 17.3%) as a yellow solid. ESI-MS: m/z=468.1 [M+1]⁺.

### Method 17

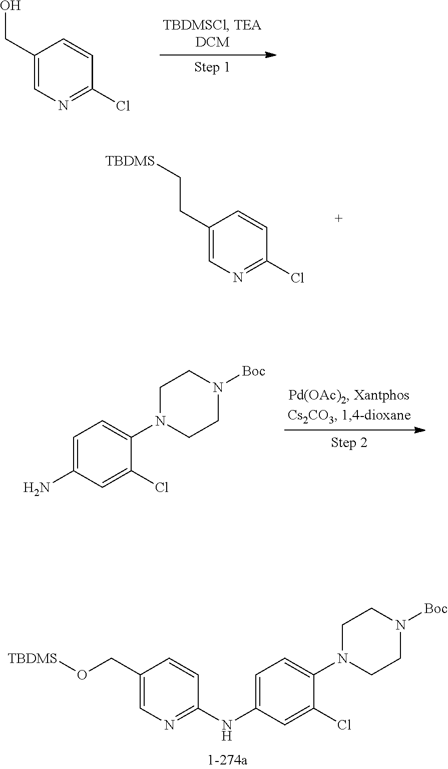

Step 1: To a solution of (6-chloro-3-pyridyl)methanol (1.0 g, 6.97 mmol) in DCM (10 mL) was added TBDMSCl (1.06 g, 7.03 mmol) and TEA (1.41 g, 13.93 mmol). The reaction mixture was stirred at 25° C. for 16 hours. The reaction mixture was poured into water (200 mL) and extracted with EtOAc (50 mL×3). The combined organic layers were washed with brine, dried over Na₂SO₄ and concentrated to afford a crude product. The residue was purified by silica gel column (EtOAc/Hexane=10:1) to give tert-butyl-[(6-chloro-3-pyridyl)methoxy]-dimethyl-silane (800 mg, 44.5% yield). ESI-MS: m/z=258.1 [M+1]⁺.
Step 2: To a solution of tert-butyl-[(6-chloro-3-pyridyl)methoxy]-dimethyl-silane (793.82 mg, 3.08 mmol) in 1,4-dioxane (15 mL) was added Cs₂CO₃ (1.67 g, 5.13 mmol), Pd(OAc)₂ (57.60 mg, 256.57 μmol), Xantphos (148.46 mg, 256.57 μmol) and tert-butyl 4-(4-amino-2-chloro-phenyl)piperazine-1-carboxylate (800 mg, 2.57 mmol). The reaction mixture was stirred at 100° C. for 18 hours under N₂. The reaction mixture was poured into water (200 mL) and extracted with EtOAc (50 mL×3). The combined organic layers were washed with brine, dried over Na₂SO₄ and concentrated to afford a crude product. The crude product was purified by silica gel chromatography (EtOAc/Hexane=1:10 to 2:8)) to give tert-butyl 4-[4-[[5-[[tert-butyl(dimethyl)silyl]oxymethyl]-2-pyridyl]amino]-2-chloro-phenyl]piperazine-1-carboxylate (1-274a, 1.09 g, 79.6% yield) as a solid. ESI-MS: m/z=533.4, 535.4 [M+1]⁺.

### Method 18

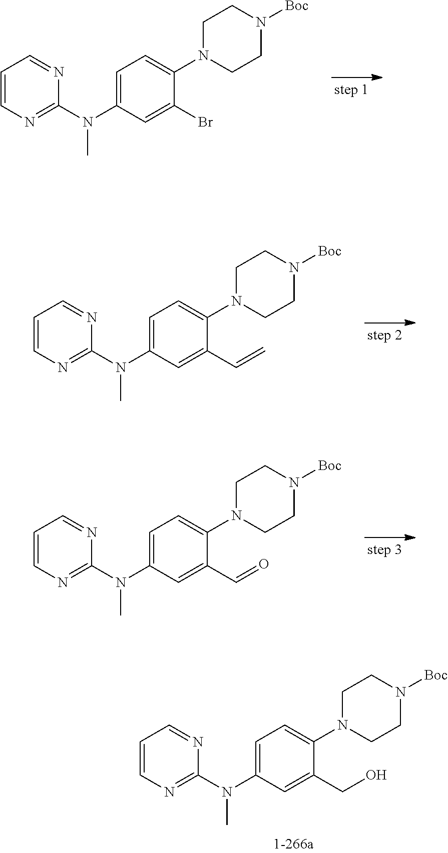 [SMILES: CN(c1ccc(N2CCN(C(=O)OC(C)(C)C)CC2)c(Br)c1)c1ncccn1 ; C=Cc1cc(N(C)c2ncccn2)ccc1N1CCN(C(=O)OC(C)(C)C)CC1 ; CN(c1ccc(N2CCN(C(=O)OC(C)(C)C)CC2)c(C=O)c1)c1ncccn1 ; CN(c1ccc(N2CCN(C(=O)OC(C)(C)C)CC2)c(CO)c1)c1ncccn1]

Step 1: A solution of tert-butyl 4-(2-bromo-4-(methyl(pyrimidin-2-yl)amino)phenyl)piperazine-1-carboxylate (1.50 g, 3.30 mmol), potassium ethenyltrifluoroboranuide (0.49 g, 3.63 mmol), Pd(dppf)Cl₂ (0.12 g, 0.10 mmol) and Cs₂CO₃ (0.91 g, 6.60 mmol) in DMF (20 mL) was stirred under nitrogen at 90° C. for 16 hours. The reaction mixture was poured into water (100 mL) and extracted with EtOAc (100 mL×3). The combined organic layers were washed with brine, dried over Na₂SO₄ and concentrated to afford a crude product. The crude product was purified by silica gel chromatography (PE/EtOAc=20/1˜5/1) to give tert-butyl 4-(4-(methyl(pyrimidin-2-yl)amino)-2-vinylphenyl)piperazine-1-carboxylate (1.10 g, 83.13% yield) as a yellow solid. ESI-MS: m/z=396.3 [M+1]⁺.
Step 2: To a stirred solution of tert-butyl 4-(4-(methyl(pyrimidin-2-yl)amino)-2-vinylphenyl)piperazine-1-carboxylate (1.1 g, 0.0028 mol) in THF/H₂O (3/1, 10 mL) was added potassium osmium dihydrate (0.02 g, 0.056 mmol) at 0° C. The mixture was stirred at 0° C. for 15 minutes. Then NaIO₄ (1.80 g, 0.0084 mol) was added. The reaction was stirred at 25° C. for 4 hours. The mixture was filtered. The filtrate was concentrated and poured into water (30 mL) and extracted with EtOAc (20 mL×3). The combined organic layers were washed with brine, dried over Na₂SO₄ and concentrated to afford a crude product. The crude product was purified by silica gel chromatography (PE/EtOAc=20/1˜5/1) to give tert-butyl 4-(2-formyl-4-(methyl(pyrimidin-2-yl)amino)phenyl)piperazine-1-carboxylate (0.60 g, 54.27% yield) as a yellow solid. ESI-MS: m/z=398.3 [M+1]⁺.
Step 3: A solution of tert-butyl 4-(2-formyl-4-(methyl(pyrimidin-2-yl)amino)phenyl)piperazine-1-carboxylate (100.0 mg, 0.25 mmol), NaBH₄ (19.0 mg, 0.50 mmol) in EtOH (3 mL) was stirred at 25° C. for 1 hour. The mixture was filtered. The filtrate was concentrated to afford a crude product. The crude product was purified by Prep-TLC (DCM/MeOH=20/1) to give tert-butyl 4-(2-(hydroxymethyl)-4-(methyl(pyrimidin-2-yl)amino)phenyl)piperazine-1-carboxylate (1-266a, 90.0 mg, 89.55% yield) as a yellow oil. ESI-MS: m/z=400.4 [M+1]⁺.

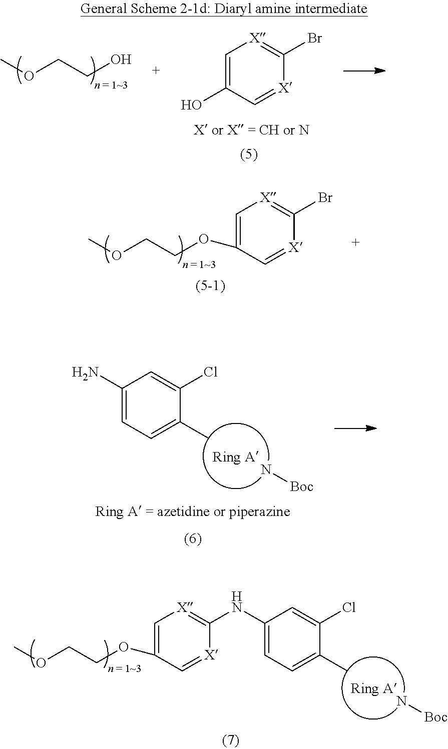

### Method 19

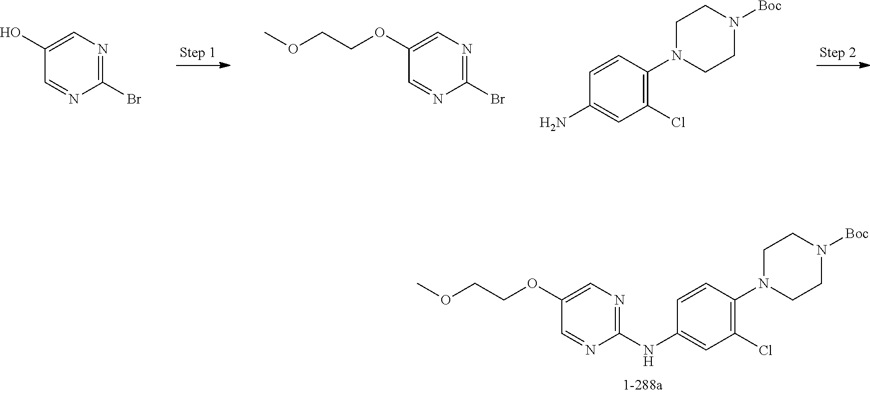 [SMILES: Oc1cnc(Br)nc1 ; COCCOc1cnc(Br)nc1 ; CC(C)(C)OC(=O)N1CCN(c2ccc(N)cc2Cl)CC1 ; COCCOc1cnc(Nc2ccc(N3CCN(C(=O)OC(C)(C)C)CC3)c(Cl)c2)nc1]

Step 1: To a mixture of 2-methoxyethanol (0.44 mL, 5.58 mmol), 2-bromopyrimidin-5-ol (490 mg, 2.80 mmol), and triphenylphosphine (735 mg, 2.80 mmol) in toluene (8.0 mL) was added bis(2-methoxyethyl) azodicarboxylate (656 mg, 2.80 mmol). The mixture was degassed via sparging with N₂ for 5 minutes and heated to 100° C. while stirring overnight. The mixture was poured over H₂O and extracted with EtOAc. The combined organic layers were washed with iN aq. NaOH solution (2 times), washed with H₂O, washed with brine, dried over Na₂SO₄, filtered, and concentrated. The residue was purified by FCC on SiO₂ using a gradient of 30-70% EtOAc in heptanes. 2-bromo-5-(2-methoxyethoxy)pyrimidine was collected as a white solid in 70% yield (456 mg, 1.96 mmol). TLC (50% EtOAc/heptanes): R_{f}=0.23, TLC (EtOAc): R_{f}=0.49. LC/MS found 232.9 [M+H]⁺.
Step 2: A mixture of 2-bromo-5-(2-methoxyethoxy)pyrimidine (100 mg, 0.43 mmol), tert-butyl 4-(4-amino-2-chloro-phenyl)piperazine-1-carboxylate (136 mg, 0.44 mmol), methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II) (20 mg, 22 μmol), 2-(dicyclohexylphosphino)-3,6-dimethoxy-2′,4′,6′-tri-i-propyl-1,1′-biphenyl (12 mg, 22 μmol), and cesium carbonate (293 mg, 0.90 mmol) in dry 1,4-dioxane (4.0 mL) was degassed via sparging with N₂ for 10 minutes then heated to 100° C. with stirring overnight. The mixture was cooled to room temperature and filtered through a pad of diatomaceous earth, and the filtrate was concentrated to dryness. The residue was purified by flash column chromatography using a gradient of 30-70% EtOAc in hexanes. 1-288a was collected as a light tan colored solid in 81% yield (1-288a, 162 mg, 0.35 mmol). TLC (50% EtOAc/hexanes): R_{f}=0.18. LC/MS found 464.1 [M+H]⁺.

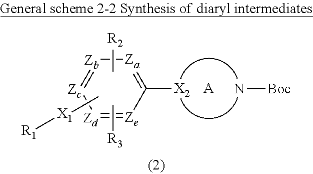

### Method 20

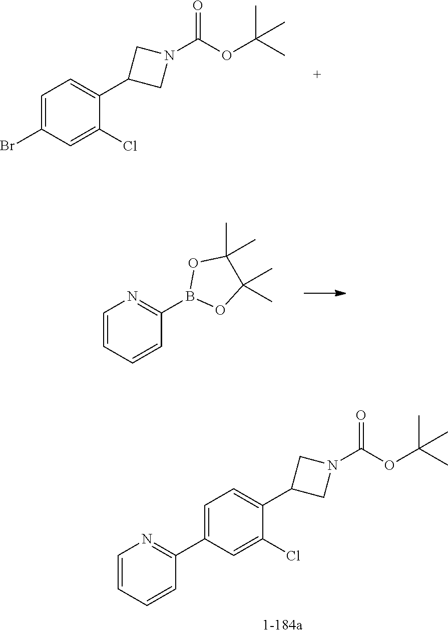 [SMILES: CC(C)(C)OC(=O)N1CC(c2ccc(Br)cc2Cl)C1 ; CC1(C)OB(c2ccccn2)OC1(C)C ; CC(C)(C)OC(=O)N1CC(c2ccc(-c3ccccn3)cc2Cl)C1]

To a solution of tert-butyl 3-(4-bromo-2-chloro-phenyl)azetidine-1-carboxylate (400 mg, 1.15 mmol) in 1,4-dioxane/H₂O (3/1, 12 mL) was added 2-(4,4,5,5-tetramethyl-1,3,2-dioxaborolan-2-yl)pyridine (354.93 mg, 1.73 mmol), Na₂CO₃ (2 M, 3.46 mmol, 1.73 mL, water), and Pd(dppf)Cl₂ (70 mg, 115.39 mol). The reaction mixture was stirred at 85° C. for 12 hours under N₂. The reaction mixture was poured into water (20 mL) and extracted with EtOAc (20 mL×3). The combined organic layers were washed with brine, dried over Na₂SO₄, and concentrated to afford a crude product. The crude product was purified by a flash column to give tert-butyl 3-[2-chloro-4-(2-pyridyl)phenyl]azetidine-1-carboxylate (1-184a, 193 mg, 559.69 μmol, 48.5% yield) as a white solid. ESI-MS: m/z=388.2 [M+1]⁺

### Synthesis of Cyclic Amine Substitution on Aryl-1

### Method 21

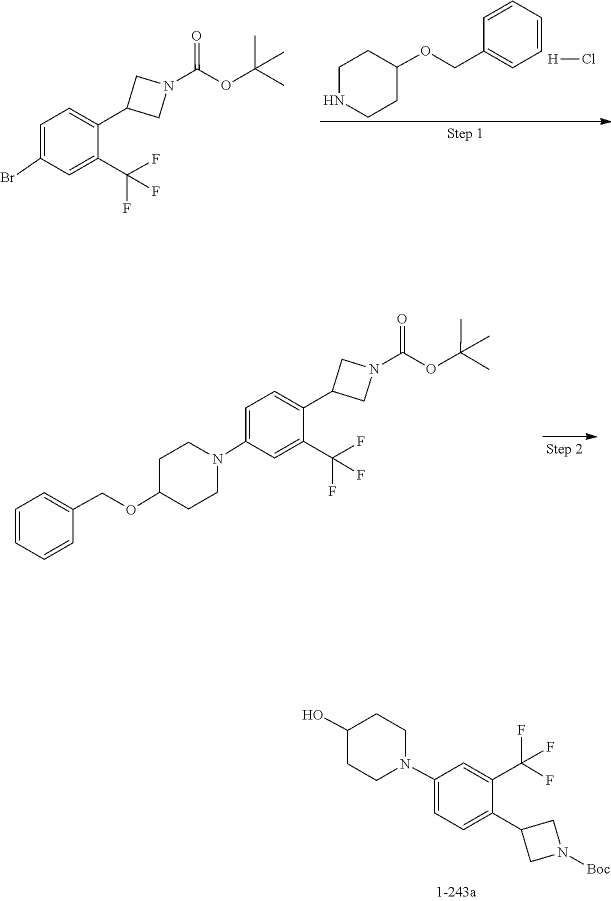

Step 1: A mixture of tert-butyl 3-[4-bromo-2-(trifluoromethyl)phenyl]azetidine-1-carboxylate (510 mg, 1.34 mmol), 4-benzyloxypiperidine;hydrochloride (397.12 mg, 1.74 mmol), Pd(OAc)₂ (15.06 mg, 67.07 μmol, 6.88 μL), Cs₂CO₃ (1.31 g, 4.02 mmol), and Xantphos (54.33 mg, 93.90 μmol) in dioxane (10.16 mL) was purged with nitrogen gas for 5 minutes. The mixture was heated to 100° C. and shaken overnight. The mixture was cooled to room temperature and diluted with EtOAc (˜30 mL) and brine (˜20 mL). The organic phase was separated, washed with brine (˜20 mL), dried over sodium sulfate, filtered, concentrated, and purified by a flash column to afford tert-butyl 3-[4-(4-benzyloxy-1-piperidyl)-2-(trifluoromethyl)phenyl]azetidine-1-carboxylate (500 mg, 1.02 mmol, 75.98% yield). EtOAc (10% to 100%) in Hexane. ESI-MS: m/z=491.2 [M+1]⁺
   Step 2: A solution of tert-butyl 3-[4-(4-benzyloxy-1-piperidyl)-2-(trifluoromethyl)phenyl]azetidine-1-carboxylate (95 mg, 193.66 μmol) in Methanol (3 mL) was treated with Pd/C (10% on Carbon (wetted with ca. 55% Water)) (20.61 mg, 193.66 μmol) was stirred under a Hydrogen gas balloon. The mixture was stirred for 6 hours at room temperature. The mixture was filtered through a cellite, and the filtrated was collected, concentrated, and purified by a flash column to afford tert-butyl 3-[4-(4-hydroxy-1-piperidyl)-2-(trifluoromethyl)phenyl]azetidine-1-carboxylate (1-243a, 69 mg, 172.31 μmol, 89% yield). EtOAc (10% to 100%) in Hexane. ESI-MS: m/z=401.4 [M+1]⁺

### Cyclic Amine Substitution on Aryl

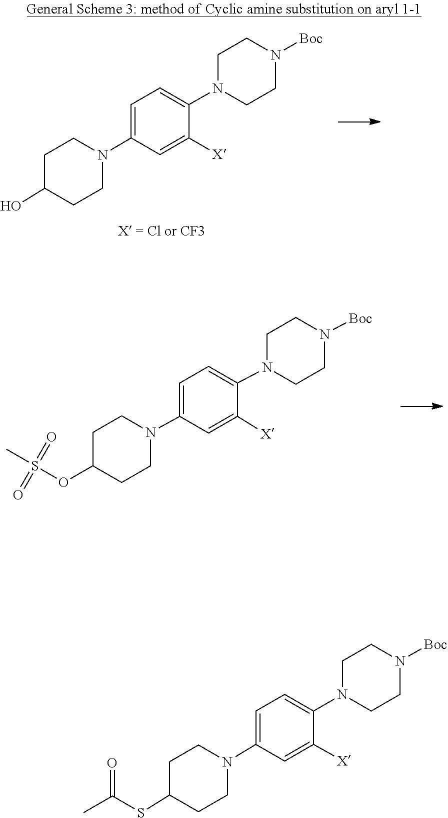

### Method 22

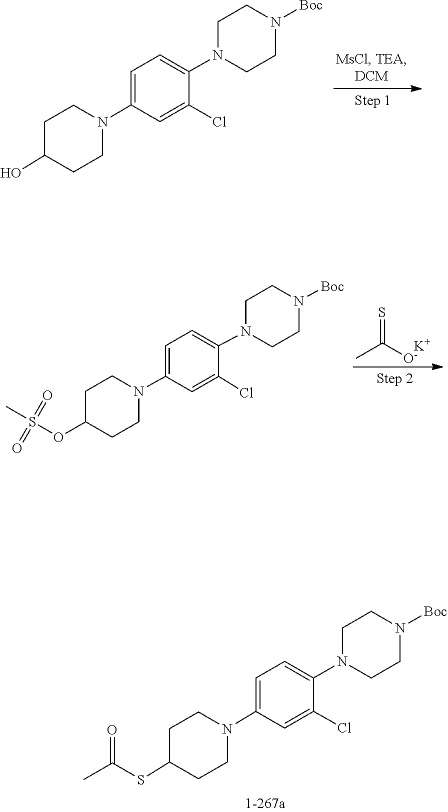

Step 1: To a solution of tert-butyl 4-(2-chloro-4-(4-hydroxypiperidin-1-yl) phenyl) piperazine-1-carboxylate (900 mg, 2.27 mmol) and TEA (690 mg, 6.82 mmol) in DCM (10 mL) was added MsCl (390 mg, 3.41 mmol) dropwise at 0° C. The mixture was stirred at 25° C. for 4 hours. The reaction mixture was poured into water (20 mL) and extracted with DCM (10 mL×3). The combined organic layers were washed with brine, dried over Na₂SO₄ and concentrated to afford a crude product. The crude product was purified by prep-TLC (PE/EtOAc=5/1) to give tert-butyl 4-(2-chloro-4-(4-((methylsulfonyl)oxy) piperidin-1-yl) phenyl) piperazine-1-carboxylate (840.0 mg, 70.10% yield) as a yellow oil. ESI-MS: m/z=473.9[M+1]⁺
Step 2: To a solution of tert-butyl 4-(2-chloro-4-(4-((methylsulfonyl)oxy) piperidin-1-yl) phenyl) piperazine-1-carboxylate (800.0 mg, 1.68 mmol) in DMF (10 mL) was added Potassium thioacetate (577 mg, 5.05 mmol). The reaction mixture was stirred at 90° C. for 4 hours. The reaction mixture was poured into water (50 mL) and extracted with EtOAc (30 mL×3). The combined organic layers were washed with brine, dried over Na₂SO₄ and concentrated to afford a crude product. The crude product was purified by TLC (PE/EtOAc=3/1) to give tert-butyl 4-(4-(4-(acetylthio) piperidin-1-yl)-2-chlorophenyl) piperazine-1-carboxylate (1-267a, 600 mg, 70.47% yield) as a pink solid. ESI-MS: m/z=454.2[M+1]⁺

### Cyclic Amine Substitution on Aryl-2

### Method 23

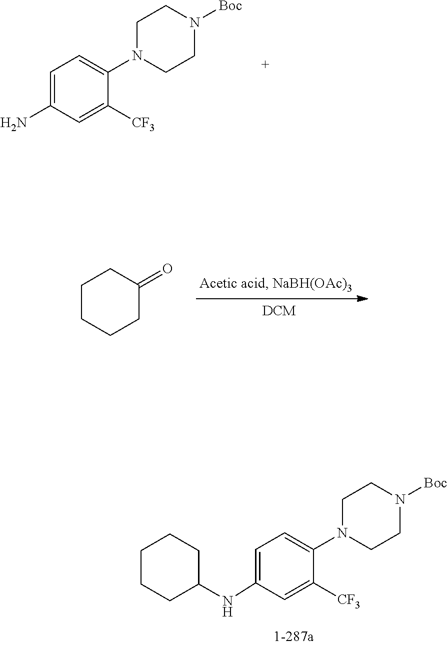 [SMILES: CC(C)(C)OC(=O)N1CCN(c2ccc(N)cc2C(F)(F)F)CC1 ; O=C1CCCCC1 ; CC(C)(C)OC(=O)N1CCN(c2ccc(NC3CCCCC3)cc2C(F)(F)F)CC1]

A solution of tert-butyl tert-butyl 4-[4-amino-2-(trifluoromethyl)phenyl]piperazine-1-carboxylate (300 mg, 868.66 μmmol) in DCM (10 mL) was stirred at room temperature. Cyclohexanone (170.51 mg, 1.74 mmol) added and stirred at room temperature for 18 hours. The reaction mixture was poured into water (100 mL) and extracted with EtOAc (100 mL×3). The combined organic layers were washed with brine, dried over Na₂SO₄ and concentrated to afford a crude product (1-287a, 371.35 mg). The crude product was used in the next reaction without purification.

### Method 24

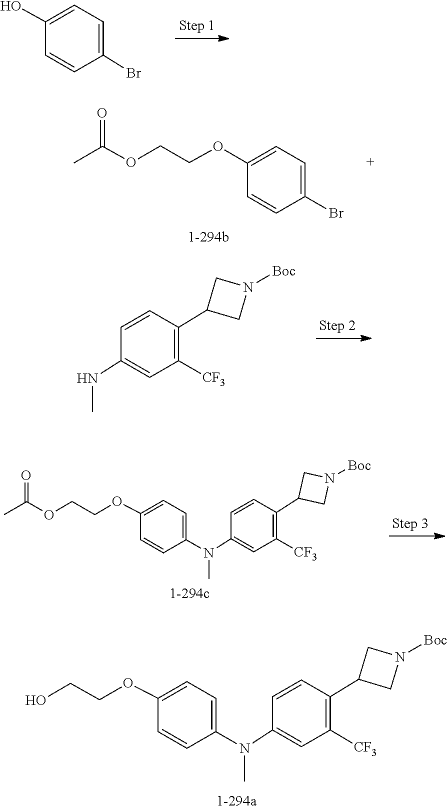 [SMILES: Oc1ccc(Br)cc1 ; CC(=O)OCCOc1ccc(Br)cc1 ; CNc1ccc(C2CN(C(=O)OC(C)(C)C)C2)c(C(F)(F)F)c1 ; CC(=O)OCCOc1ccc(N(C)c2ccc(C3CN(C(=O)OC(C)(C)C)C3)c(C(F)(F)F)c2)cc1 ; CN(c1ccc(OCCO)cc1)c1ccc(C2CN(C(=O)OC(C)(C)C)C2)c(C(F)(F)F)c1]

Step 1: To a mixture of 2-hydroxyethyl acetate (3.00 g, 28.8 mmol), 4-bromophenol (5.48 g, 31.7 mmol), and diphenyl-2-pyridylphosphine (8.00 g, 30.4 mmol) in toluene (60 mL) was added bis(2-methoxyethyl) azodicarboxylate (7.00 g, 29.9 mmol) at 0° C. The mixture was allowed to gradually warm to room temperature and stirred overnight. The mixture was diluted with EtOAc and washed with 3N aq. HCl solution (2 times), washed with H₂O, washed with brine, dried over Na₂SO₄, filtered, and concentrated. The residue was purified by FCC on SiO₂ using a gradient of 10-100% CH₂Cl₂ in heptanes. 1-294b was collected as a colorless oil in 32% yield (2.36 g, 9.11 mmol). TLC (10% EtOAc/heptanes): R_{f}=0.22, TLC (30% EtOAc/heptanes): R_{f}=0.49, TLC (CH₂Cl₂): R_{f}=0.61. LC/MS found 259.0 [M+H]⁺.
Step 2: A mixture of 2-(4-bromophenoxy)ethyl acetate (919 mg, 3.55 mmol), tert-butyl 3-[4-(methylamino)-2-(trifluoromethyl)phenyl]azetidine-1-carboxylate (1.23 g, 3.72 mmol), methanesulfonato(2-dicyclohexylphosphino-2′,6′-di-i-propoxy-1,1′-biphenyl)(2′-methylamino-1,1′-biphenyl-2-yl)palladium(II) (75 mg, 88 μmol), 2-dicyclohexylphosphino-2′,6′-di-i-propoxy-1,1′-biphenyl (41 mg, 88 μmol), and cesium carbonate (2.31 g, 7.09 mmol) in dry 1,4-dioxane (10 mL) was degassed via sparging with N₂ for 10 minutes then heated to 80° C. with stirring for 4 hours. The mixture was cooled to room temperature and filtered through a pad of diatomaceous earth, and the filtrate was concentrated to dryness. The residue was purified by flash column chromatography using a gradient of 10-40% EtOAc in hexanes. 1-294c was collected as a viscous yellow oil in 65% yield (1.17 g, 2.30 mmol). TLC (30% EtOAc/hexanes): R_{f}=0.32, TLC (5% EtOAc/CH₂Cl₂): R_{f}=0.33, fluoresces blue under long wave UV. LC/MS found 509.3 [M+H]⁺. 453.3 [M+H−t−Bu]⁺, and 409.2 [M+H−Boc]⁺.
Step 3: A mixture of tert-butyl 3-[4-[4-(2-acetoxyethoxy)-N-methyl-anilino]-2-(trifluoromethyl)phenyl]azetidine-1-carboxylate (1.17 g, 2.30 mmol) in THF (10 mL) and aq. sodium hydroxide solution (3N, 30 mmol, 10 mL, water) was heated to 40° C. with stirring overnight. The mixture was cooled to room temperature, poured over H₂O, and extracted with EtOAc. The combined organic layers were washed with H₂O, washed with brine, dried over Na₂SO₄, filtered, and concentrated. The residue was purified by FCC on SiO₂ using a gradient of 30-60% EtOAc in heptanes. 1-294a was collected as a viscous orange oil in 99% yield (1.06 g, 2.27 mmol). TLC (50% EtOAc/heptanes): R_{f}=0.29. LC/MS found 467.3 [M+H]⁺, 411.2 [M+H−t−Bu]⁺, and 367.2 [M+H−Boc]⁺.

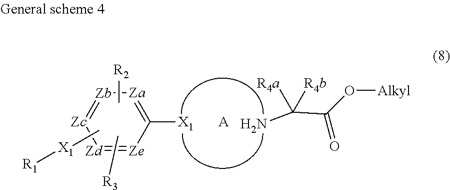

### Method 25

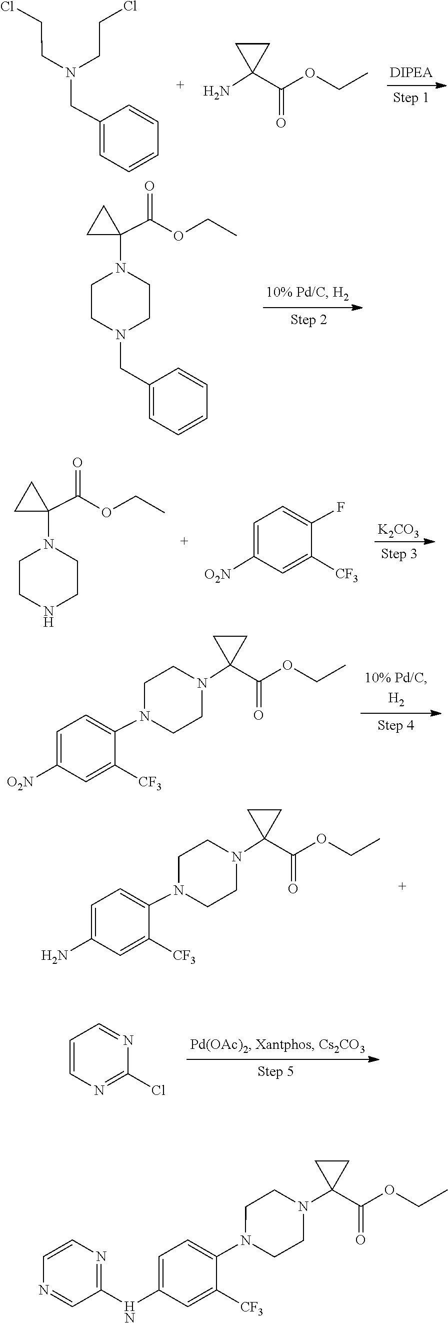

Step 1: A mixture of N-benzyl-2-chloro-N-(2-chloroethyl)ethanamine hydrochloride (10 g, 37.23 mmol), ethyl 1-aminocyclopropanecarboxylate (5.29 g, 40.95 mmol), and N,N-Diisopropylethylamine (64.85 mL, 372.29 mmol) in ethanol (50 mL) was stirred at reflux for 18 hours. The reaction mixture was concentrated to dryness. The residue was partitioned between dichloromethane and water. Two layers were separated, and the aqueous layer was extracted with dichloromethane. The combined organic layers were concentrated. The residue was purified by silica gel column (EtOAc/Hexane=1:5) to give ethyl 1-(4-benzylpiperazin-1-yl)cyclopropanecarboxylate (5.15 g, 47% yield) as a yellow oil. ESI-MS: m/z=289.3 [M+1]⁺.
Step 2: A mixture of ethyl 1-(4-benzylpiperazin-1-yl)cyclopropanecarboxylate (5.15 g, 17.86 mmol) and Pd/C (10%, 950 mg) in MeOH/EtOAc (50 mL/10 mL) was stirred at 20° C. overnight under H₂. The reaction mixture was filtered, and the filtrate concentrated to give ethyl 1-piperazin-1-ylcyclopropanecarboxylate (3.50 g, 98% yield). The crude product was used in the next step without further purification. ESI-MS: m/z=199.3 [M+1]⁺.
Step 3: A mixture of crude ethyl 1-piperazin-1-ylcyclopropanecarboxylate (3.44 g, 17.36 mmol), 1-fluoro-4-nitro-2-(trifluoromethyl)benzene (3.3 g, 15.58 mmol), and K₂CO₃ (4.36 g, 31.56 mmol) in DMF was stirred at 90° C. overnight. Then, an excess amount of water was added to the reactant, stirred, and the resulting solid compound was filtered and dried to obtain the ethyl 1-[4-[4-nitro-2-(trifluoromethyl)phenyl]piperazin-1-yl]cyclopropanecarboxylate (5.71 g, 93% yield). The crude product was used in the next step without further purification. ESI-MS: m/z=388.3 [M+1]⁺.
Step 4: A mixture of ethyl 1-[4-[4-nitro-2-(trifluoromethyl)phenyl]piperazin-1-yl]cyclopropanecarboxylate (5.71 g, 14.74 mmol) and 10% Pd/C (549 mg) in MeOH/EtOAc (50 mL/10 mL) was stirred at 20° C. overnight under H₂. The reaction mixture was filtered, and the filtrate was concentrated to give ethyl 1-(4-(4-amino-2-(trifluoromethyl)phenyl)piperazin-1-yl)cyclopropane-1-carboxylate (2.62 g, 49% yield). The crude product was used in the next step without further purification. ESI-MS: m/z=358.3 [M+1]⁺.
Step 5: To a solution of 2-chloropyrimidine (153.83 mg, 1.34 mmol) in 1,4-dioxane (10 mL) under nitrogen was added ethyl 1-(4-(4-amino-2-(trifluoromethyl)phenyl)piperazin-1-yl)cyclopropane-1-carboxylate (400 mg, 1.12 mmol), Cs₂CO₃ (729.37 mg, 2.24 mmol), Pd(OAc)₂ (25.13 mg, 111.93 μmol), and Xantphos (64.76 mg, 111.93 μmol) at 20° C. The reaction mixture was stirred at 100° C. for 18 hours. The reaction mixture was poured into water (300 mL) and extracted with EtOAc (100 mL×3). The combined organic layers were washed with brine, dried over Na₂SO₄, and concentrated to afford a crude product. The crude product was purified by silica gel chromatography (EtOAc/Hexane=1:5) to give ethyl 1-(4-(4-(pyrimidin-2-ylamino)-2-(trifluoromethyl)-phenyl)piperazin-1-yl)cyclopropane-1-carboxylate (180 mg, 36% yield) as a brown solid. ESI-MS: m/z=436.4 [M+1]⁺.

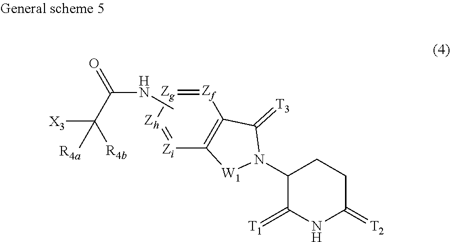

Synthesis of Amide linker intermediate

### Method 26

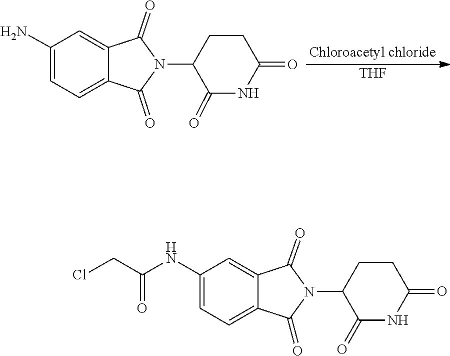 [SMILES: Nc1ccc2c(c1)C(=O)N(C1CCC(=O)NC1=O)C2=O ; O=C1CCC(N2C(=O)c3ccc(NC(=O)CCl)cc3C2=O)C(=O)N1 ; "THF"]

To a mixture of 5-amino-2-(2,6-dioxopiperidin-3-yl)isoindoline-1,3-dione (10.00 g, 36.60 mmol) in THF (100 mL) was added chloroacetyl chloride (4.96 g, 43.92 mmol). The mixture was stirred at 75° C. for 2 hours. The reaction mixture was concentrated to afford a crude product, which was stirred in MTBE (100 mL) at 25° C. for 30 minutes. The mixture was filtered, and the filter cake was dried in vacuo to afford 2-chloro-N-(2-(2,6-dioxopiperidin-3-yl)-1,3-dioxoisoindolin-5-yl)acetamide (8.00 g, 62.5% yield) as a white solid. ESI-MS: m/z=350.1 [M+1]⁺; ¹HNMR (400 MHz, DMSO-d₆) δ 11.13 (s, 1H), 10.96 (s, 1H), 8.23 (s, 1H), 7.92 (s, 2H), 5.16-5.11 (m, 1H), 4.37 (s, 2H), 2.90-2.86 (m, 1H), 2.53-2.69 (m, 2H), 2.23-2.20 (m, 1H).

### Method 27

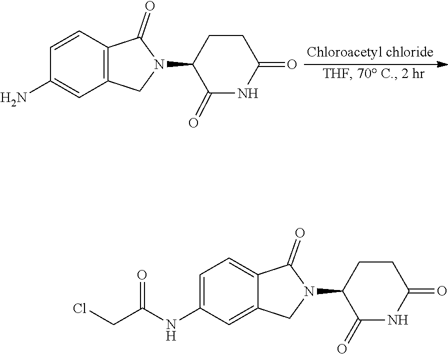

To a solution of (S)-3-(5-amino-1-oxoisoindolin-2-yl)piperidine-2,6-dione (200 mg, 0.726 mmol) in THF (10 mL) was added 2-chloroacetyl chloride (81.31 mg, 0.726 mmol) at 20° C. under N₂. The reaction mixture was stirred at 70° C. for 2 hours. The mixture was concentrated in vacuum to afford a crude product, which was stirred in MTBE (20 mL) for 30 minutes. The mixture was filtered, and the filter cake was dried to dryness to give (S)-2-chloro-N-(2-(2,6-dioxopiperidin-3-yl)-1-oxoisoindolin-5-yl)acetamide (180 mg, 95% yield) as a white solid. ESI-MS: m/z=336.1[M+1]⁺; ¹H NMR (400 MHz, DMSO-d₆) δ 10.99 (s, 1H), 10.80 (s, 1H), 7.98 (s, 1H), 7.66 (dt, J=8.2, 4.9 Hz, 2H), 5.09 (dd, J=13.2, 5.2 Hz, 1H), 4.45 (d, J=17.4 Hz, 1H), 4.35-4.27 (m, 3H), 2.97-2.83 (m, 1H), 2.60 (d, J=17.9 Hz, 1H), 2.38 (dd, J=13.2, 4.5 Hz, 1H), 2.06-1.93 (m, 1H).

### Method 28

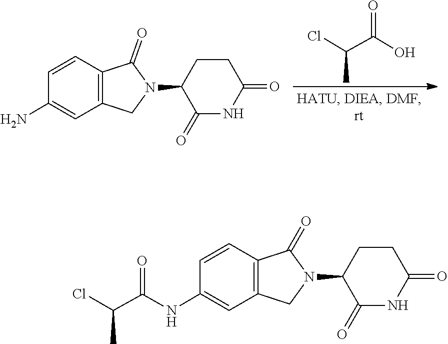

To a solution of (2R)-2-chloropropanoic acid (0.76 g, 6.90 mmol) in DMF (20 mL) was added HATU (3.31 g, 8.70 mmol) at 0° C. The reaction mixture was stirred at 0° C. for 0.5 hours. Then (S)-3-(5-amino-1-oxoisoindolin-2-yl)piperidine-2,6-dione (1.50 g, 5.80 mmol) and DIEA (2.25 g, 17.4 mmol) were added. The reaction mixture was stirred at 25° C. for 12 hours. The reaction mixture was poured into water (100 mL) and extracted with DCM (25 mL×3). The combined organic layers were washed with brine, dried over Na₂SO₄, and concentrated to afford a crude product. The crude product was purified by prep-TLC (DCM/MeOH=10/1) to give (R)-2-chloro-N-(2-((S)-2,6-dioxopiperidin-3-yl)-1-oxoisoindolin-5-yl)propanamide (1.03 g, 50.9% yield) as a yellow solid. ESI-MS: m/z=350.0 [M+1]⁺; ¹H NMR (400 MHz, DMSO_d₆) δ 10.99 (s, 1H), 10.65 (s, 1H), 7.99 (s, 1H), 7.70 (d, J=8.4 Hz, 1H), 7.62 (d, J=8.4 Hz, 1H), 5.09 (dd, J=13.2, 4.8 Hz, 1H), 4.71 (q, J=6.8 Hz, 1H), 4.45 (d, J=17.2 Hz, 1H), 4.31 (d, J=17.2 Hz, 1H), 2.97-2.85 (m, 1H), 2.67-2.58 (m, 1H), 2.40-2.32 (m, 1H), 2.04-1.95 (m, 1H), 1.63 (d, J=6.4 Hz, 3H).

### Method 29

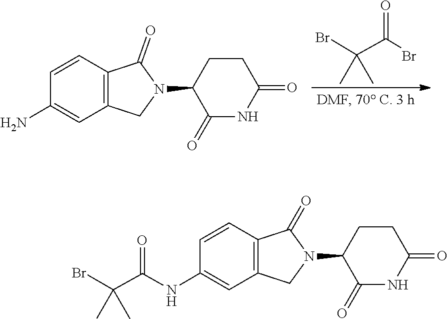

To a solution of (S)-3-(5-amino-1-oxoisoindolin-2-yl)piperidine-2,6-dione (50 mg, 192.86 μmmol) in DMF (10 mL) was added 2-bromo-2-methyl-propanoyl bromide (88.67 mg, 385.71 μmol). The reaction mixture was stirred at 70° C. for 3 hours. The mixture was concentrated in vacuum to afford a crude product, which was stirred in MTBE (20 mL) for 30 minutes. The mixture was filtered, and the filter cake was dried to dryness to give (S)-2-bromo-N-(2-(2,6-dioxopiperidin-3-yl)-1-oxoisoindolin-5-yl)-2-methylpropanamide (60 mg) as a brown solid. ESI-MS: m/z=408.2, 410.2 [M+1]⁺.

### Method 30

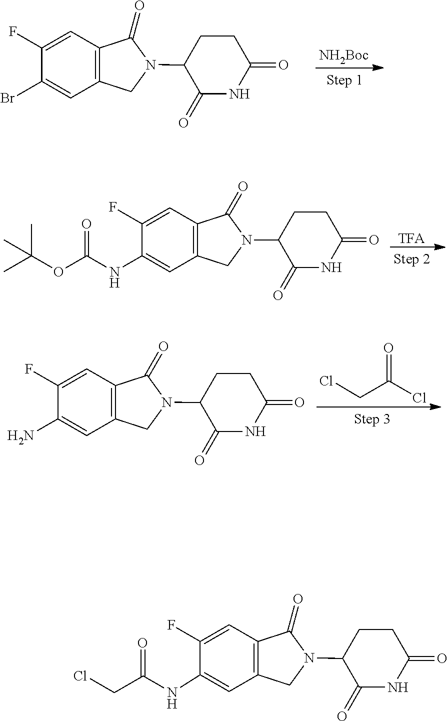

Step 1: To a solution of 3-(5-bromo-6-fluoro-1-oxoisoindolin-2-yl) piperidine-2,6-dione (20.0 mg, 0.06 mmol) in 1,4-dioxane (1.5 mL) was added tert-butyl carbamate (13.73 mg, 0.12 mmol), K₃PO₄ (37.32 mg, 0.17 mmol), and XPhos-Pd-G3 (10 mg). The reaction mixture was stirred at 100° C. for 16 hours under N₂. Repeat the above operation 50 times. Each reaction mixture was poured into water (100 mL) and extracted with EtOAc (100 mL×3). The combined organic layers were washed with brine, dried over Na₂SO₄, and concentrated to afford a crude product. The crude product was purified by prep-TLC (PE/EtOAc=1/2) to give tert-butyl (2-(2,6-dioxopiperidin-3-yl)-6-fluoro-1-oxoisoindolin-5-yl) carbamate (0.41 g, 36.0% yield) as a white solid. ESI-MS: m/z=378.2 [M+1]⁺.
Step 2: A solution of tert-butyl (2-(2,6-dioxopiperidin-3-yl)-6-fluoro-1-oxoisoindolin-5-yl) carbamate (800.0 mg, 2.12 mmol) in DCM/TFA=5/1 (12 mL) was stirred at 25° C. for 6 hours under N₂. The reaction mixture was concentrated to give 3-(5-amino-6-fluoro-1-oxoisoindolin-2-yl) piperidine-2,6-dione (600.0 mg, 81.6% yield) as a yellow solid. The residue was used in the next step without further purification. ESI-MS: m/z=278.1 [M+1]⁺.
Step 3: To a solution of 3-(5-amino-6-fluoro-1-oxoisoindolin-2-yl) piperidine-2,6-dione (300.0 mg, 1.08 mmol) in THF (4 mL) was added chloroacetyl chloride (244.42 mg, 2.16 mmol) dropwise. The reaction mixture was stirred at 70° C. for 20 minutes under N₂. The mixture was filtered. The filter cake was washed with THF and dried to dryness to give the crude product. The solid was stirred in THF (3 mL) for 30 minutes. The mixture was filtered, and the filter cake was dried to dryness to give 2-chloro-N-(2-(2,6-dioxopiperidin-3-yl)-6-fluoro-1-oxoisoindolin-5-yl) acetamide (190.0 mg, 47.0% yield) as a white solid. ESI-MS: m/z=353.9 [M+1]⁺; ¹H NMR (400 MHz, DMSO_d₆) δ 11.00 (s, 1H), 10.37 (s, 1H), 8.28 (d, J=6.6 Hz, 1H), 7.63 (d, J=9.6 Hz, 1H), 5.10 (dd, J=13.6, 5.2 Hz, 1H), 4.48-4.28 (m, 4H), 2.96-2.86 (m, 1H), 2.68-2.57 (m, 1H), 2.45-2.32 (m, 1H), 2.04-1.98 (m, 1H).

### Method 31

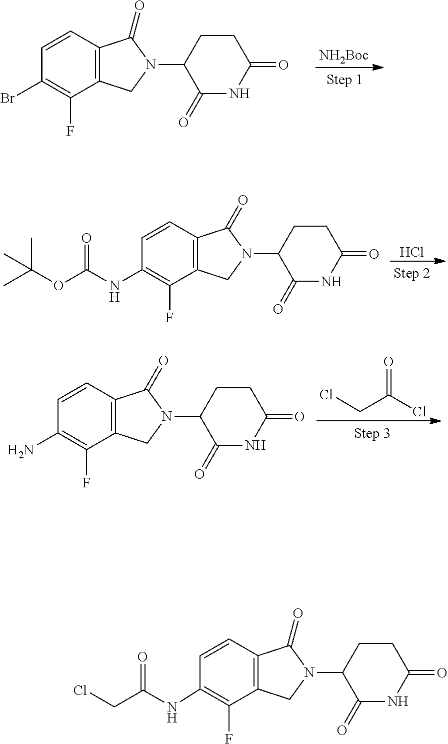

Step 1: To a solution of 3-(5-bromo-4-fluoro-1-oxoisoindolin-2-yl) piperidine-2,6-dione (1.00 g, 2.94 mmol) in 1,4-dioxane (10 mL) was added K₃PO₄ (1.9 g, 8.82 mmol), t-BuXPhos Pd G3 (1 g), and tert-Butyl carbamate (416.3 mg, 3.53 mmol). The reaction mixture was stirred at 120° C. for 16 hours under N₂. The reaction mixture was poured into water (50 mL) and extracted with EtOAc (20 mL×3). The combined organic layers were washed with brine, dried over Na₂SO₄, and concentrated to afford a crude product. The crude product was purified by prep-TLC (PE/EtOAc=3/1) to give tert-butyl (2-(2,6-dioxopiperidin-3-yl)-4-fluoro-1-oxoisoindolin-5-yl) carbamate (0.9 g, 81.8% yield) as a gray solid.
Step 2: A solution of tert-butyl (2-(2,6-dioxopiperidin-3-yl)-4-fluoro-1-oxoisoindolin-5-yl) carbamate (0.9 g, 2.39 mmol) in 1,4-dioxane/HCl (4 mol/L, 40 mL) was stirred at 25° C. for 16 hours. The reaction mixture was concentrated to afford a crude product, which was stirred in MTBE (5 mL) for 30 minutes. The mixture was filtered, and the filter cake was dried to dryness to give 3-(5-amino-4-fluoro-1-oxoisoindolin-2-yl) piperidine-2,6-dione (450 mg, 87.5% yield) as a yellow solid. ESI-MS: m/z=278.1[M+1]⁺.
Step 3: A solution of 3-(5-amino-4-fluoro-1-oxoisoindolin-2-yl) piperidine-2,6-dione (450 mg, 1.62 mmol) and 2-chloroacetyl chloride (272 mg, 1.95 mmol) in THF (5 mL) was stirred at 80° C. for 1 hour. The mixture was filtered. The filter cake was dried to dryness to give 2-chloro-N-(2-(2,6-dioxopiperidin-3-yl)-4-fluoro-1-oxoisoindolin-5-yl) acetamide (120 mg, 25.1% yield) as a white solid. ESI-MS: m/z=353.8 [M+1]⁺; ¹H NMR (400 MHz, DMSO_d₆) δ 11.02 (s, 1H), 10.41 (s, 1H), 8.08 (t, J=6.8 Hz, 1H), 7.59 (d, J=8.2 Hz, 1H), 5.11 (dd, J=13.3, 5.0 Hz, 1H), 4.63-4.55 (m, 1H), 4.46-4.34 (m, 3H), 2.98-2.85 (m, 1H), 2.69-2.85 (m, 1H), 2.46-2.37 (m, 1H), 2.06-1.95 (m, 1H).

The following compounds (1) 1-1 to 1-294 shown in Table 1 were prepared by methods similar to those described for the preparation of compound 1-4, 1-56, 1-168, or more using the appropriate Boc (2)-amine and amide intermediate (4).

TABLE 1

| Compd | 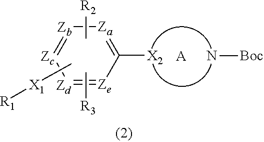 | 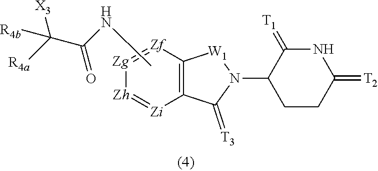 |
|---|---|---|
| 1-1 | 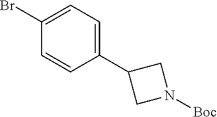 [SMILES: CC(C)(C)OC(=O)N1CC(c2ccc(Br)cc2)C1] | 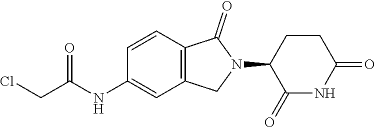 [SMILES: O=C1CC[C@H](N2Cc3cc(NC(=O)CCl)ccc3C2=O)C(=O)N1] |
| 1-2 | 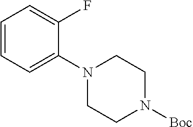 [SMILES: CC(C)(C)OC(=O)N1CCN(c2ccccc2F)CC1] | 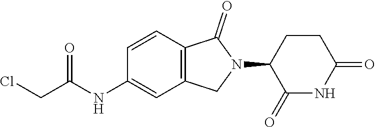 [SMILES: O=C1CC[C@H](N2Cc3cc(NC(=O)CCl)ccc3C2=O)C(=O)N1] |
| 1-3 | 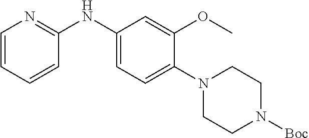 [SMILES: COc1cc(Nc2ccccn2)ccc1N1CCN(C(=O)OC(C)(C)C)CC1] | 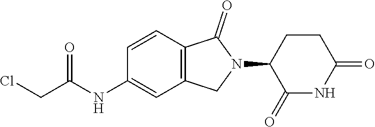 [SMILES: O=C1CC[C@H](N2Cc3cc(NC(=O)CCl)ccc3C2=O)C(=O)N1] |
| 1-4 | 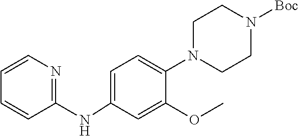 [SMILES: COc1cc(Nc2ccccn2)ccc1N1CCN(C(=O)OC(C)(C)C)CC1] | 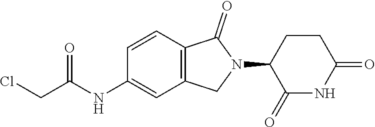 [SMILES: O=C1CC[C@H](N2Cc3cc(NC(=O)CCl)ccc3C2=O)C(=O)N1] |
| 1-5 | 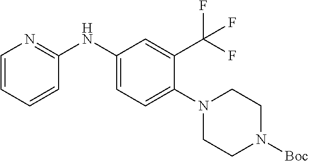 [SMILES: CC(C)(C)OC(=O)N1CCN(c2ccc(Nc3ccccn3)cc2C(F)(F)F)CC1] | 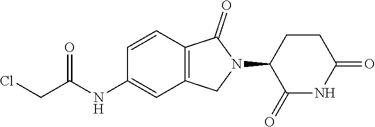 [SMILES: O=C1CC[C@H](N2Cc3cc(NC(=O)CCl)ccc3C2=O)C(=O)N1] |
| 1-6 | 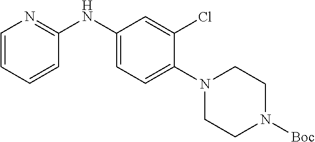 [SMILES: CC(C)(C)OC(=O)N1CCN(c2ccc(Nc3ccccn3)cc2Cl)CC1] | 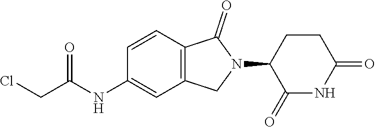 [SMILES: O=C1CC[C@H](N2Cc3cc(NC(=O)CCl)ccc3C2=O)C(=O)N1] |
| 1-7 | 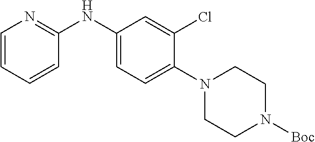 [SMILES: CC(C)(C)OC(=O)N1CCN(c2ccc(Nc3ccccn3)cc2Cl)CC1] | 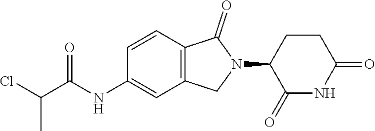 [SMILES: CC(Cl)C(=O)Nc1ccc2c(c1)CN([C@H]1CCC(=O)NC1=O)C2=O] |
| 1-8 | 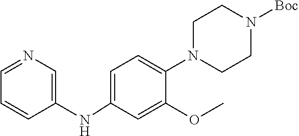 [SMILES: COc1cc(Nc2cccnc2)ccc1N1CCN(C(=O)OC(C)(C)C)CC1] | 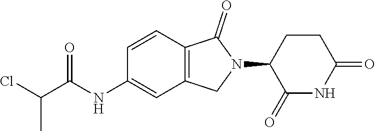 [SMILES: CC(Cl)C(=O)Nc1ccc2c(c1)CN([C@H]1CCC(=O)NC1=O)C2=O] |
| 1-9 | 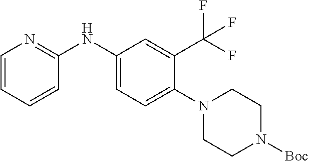 [SMILES: CC(C)(C)OC(=O)N1CCN(c2ccc(Nc3ccccn3)cc2C(F)(F)F)CC1] | 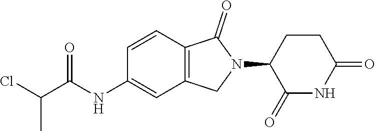 [SMILES: CC(Cl)C(=O)Nc1ccc2c(c1)CN([C@H]1CCC(=O)NC1=O)C2=O] |
| 1-10 | 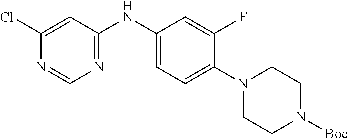 [SMILES: CC(C)(C)OC(=O)N1CCN(c2ccc(Nc3cc(Cl)ncn3)cc2F)CC1] | 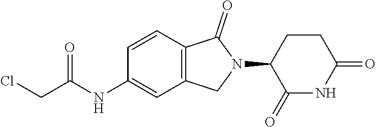 [SMILES: O=C1CC[C@H](N2Cc3cc(NC(=O)CCl)ccc3C2=O)C(=O)N1] |
| 1-11 | 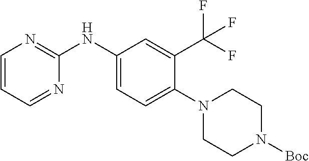 [SMILES: CC(C)(C)OC(=O)N1CCN(c2ccc(Nc3ncccn3)cc2C(F)(F)F)CC1] | 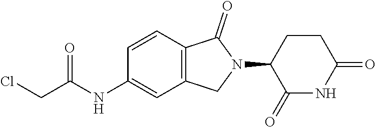 [SMILES: O=C1CC[C@H](N2Cc3cc(NC(=O)CCl)ccc3C2=O)C(=O)N1] |
| 1-12 | 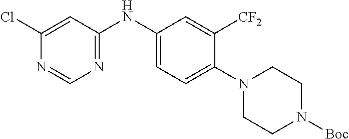 | 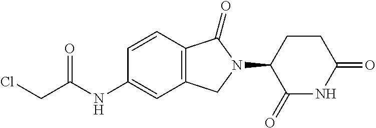 [SMILES: O=C1CC[C@H](N2Cc3cc(NC(=O)CCl)ccc3C2=O)C(=O)N1] |
| 1-13 | 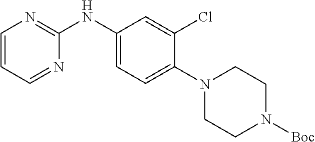 [SMILES: CC(C)(C)OC(=O)N1CCN(c2ccc(Nc3ncccn3)cc2Cl)CC1] | 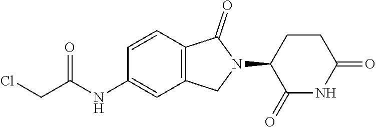 [SMILES: O=C1CC[C@H](N2Cc3cc(NC(=O)CCl)ccc3C2=O)C(=O)N1] |
| 1-14 | 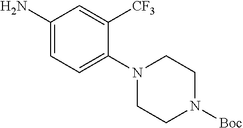 [SMILES: CC(C)(C)OC(=O)N1CCN(c2ccc(N)cc2C(F)(F)F)CC1] | 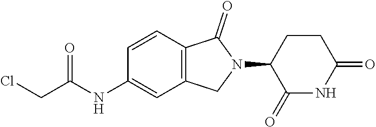 [SMILES: O=C1CC[C@H](N2Cc3cc(NC(=O)CCl)ccc3C2=O)C(=O)N1] |
| 1-15 | 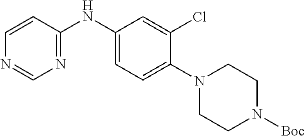 [SMILES: CC(C)(C)OC(=O)N1CCN(c2ccc(Nc3ccncn3)cc2Cl)CC1] | 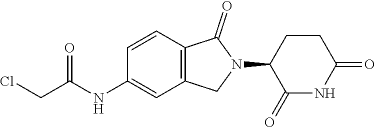 [SMILES: O=C1CC[C@H](N2Cc3cc(NC(=O)CCl)ccc3C2=O)C(=O)N1] |
| 1-16 | 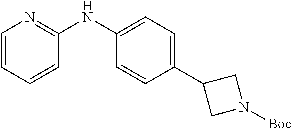 [SMILES: CC(C)(C)OC(=O)N1CC(c2ccc(Nc3ccccn3)cc2)C1] | 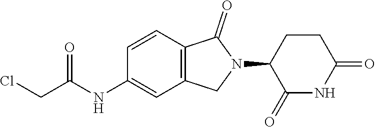 [SMILES: O=C1CC[C@H](N2Cc3cc(NC(=O)CCl)ccc3C2=O)C(=O)N1] |
| 1-17 | 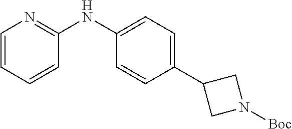 [SMILES: CC(C)(C)OC(=O)N1CC(c2ccc(Nc3ccccn3)cc2)C1] | 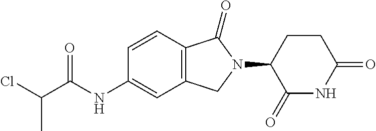 [SMILES: CC(Cl)C(=O)Nc1ccc2c(c1)CN([C@H]1CCC(=O)NC1=O)C2=O] |
| 1-18 | 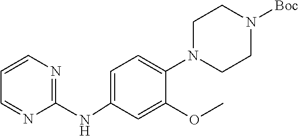 [SMILES: COc1cc(Nc2ncccn2)ccc1N1CCN(C(=O)OC(C)(C)C)CC1] | 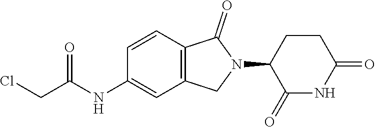 [SMILES: O=C1CC[C@H](N2Cc3cc(NC(=O)CCl)ccc3C2=O)C(=O)N1] |
| 1-19 | 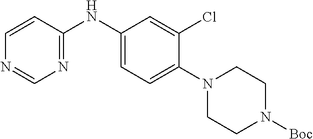 [SMILES: CC(C)(C)OC(=O)N1CCN(c2ccc(Nc3ccncn3)cc2Cl)CC1] | 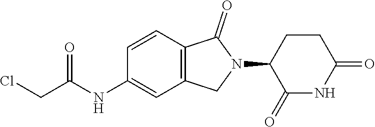 [SMILES: O=C1CC[C@H](N2Cc3cc(NC(=O)CCl)ccc3C2=O)C(=O)N1] |
| 1-20 | 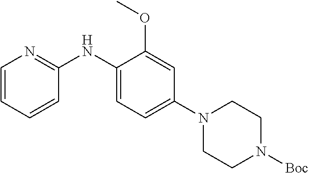 [SMILES: COc1cc(N2CCN(C(=O)OC(C)(C)C)CC2)ccc1Nc1ccccn1] | 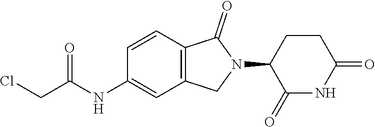 [SMILES: O=C1CC[C@H](N2Cc3cc(NC(=O)CCl)ccc3C2=O)C(=O)N1] |
| 1-21 | 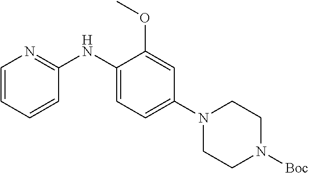 [SMILES: COc1cc(N2CCN(C(=O)OC(C)(C)C)CC2)ccc1Nc1ccccn1] | 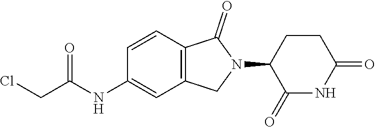 [SMILES: O=C1CC[C@H](N2Cc3cc(NC(=O)CCl)ccc3C2=O)C(=O)N1] |
| 1-22 | 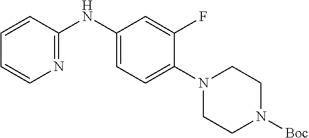 [SMILES: CC(C)(C)OC(=O)N1CCN(c2ccc(Nc3ccccn3)cc2F)CC1] | 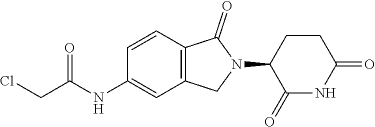 [SMILES: O=C1CC[C@H](N2Cc3cc(NC(=O)CCl)ccc3C2=O)C(=O)N1] |
| 1-23 | 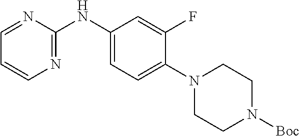 [SMILES: CC(C)(C)OC(=O)N1CCN(c2ccc(Nc3ncccn3)cc2F)CC1] | 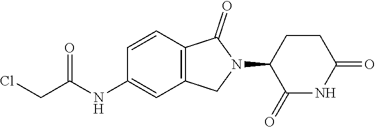 [SMILES: O=C1CC[C@H](N2Cc3cc(NC(=O)CCl)ccc3C2=O)C(=O)N1] |
| 1-24 | 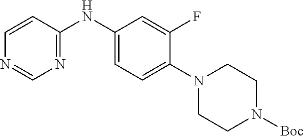 [SMILES: CC(C)(C)OC(=O)N1CCN(c2ccc(Nc3ccncn3)cc2F)CC1] | 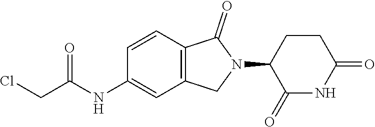 [SMILES: O=C1CC[C@H](N2Cc3cc(NC(=O)CCl)ccc3C2=O)C(=O)N1] |
| 1-25 | 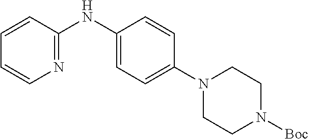 [SMILES: CC(C)(C)OC(=O)N1CCN(c2ccc(Nc3ccccn3)cc2)CC1] | 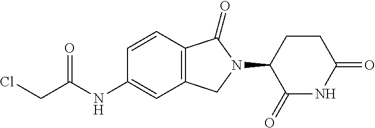 [SMILES: O=C1CC[C@H](N2Cc3cc(NC(=O)CCl)ccc3C2=O)C(=O)N1] |
| 1-26 | 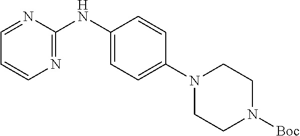 [SMILES: CC(C)(C)OC(=O)N1CCN(c2ccc(Nc3ncccn3)cc2)CC1] | 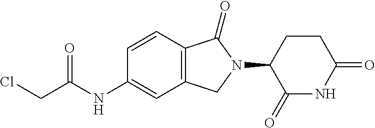 [SMILES: O=C1CC[C@H](N2Cc3cc(NC(=O)CCl)ccc3C2=O)C(=O)N1] |
| 1-27 | 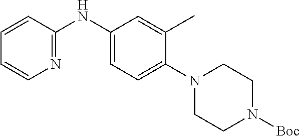 [SMILES: Cc1cc(Nc2ccccn2)ccc1N1CCN(C(=O)OC(C)(C)C)CC1] | 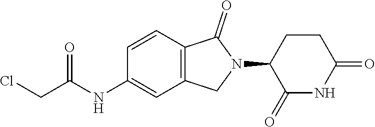 [SMILES: O=C1CC[C@H](N2Cc3cc(NC(=O)CCl)ccc3C2=O)C(=O)N1] |
| 1-28 | 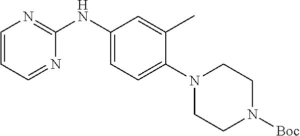 [SMILES: Cc1cc(Nc2ncccn2)ccc1N1CCN(C(=O)OC(C)(C)C)CC1] | 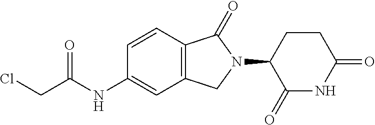 [SMILES: O=C1CC[C@H](N2Cc3cc(NC(=O)CCl)ccc3C2=O)C(=O)N1] |
| 1-29 | 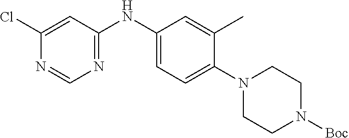 [SMILES: Cc1cc(Nc2cc(Cl)ncn2)ccc1N1CCN(C(=O)OC(C)(C)C)CC1] | 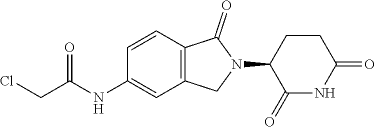 [SMILES: O=C1CC[C@H](N2Cc3cc(NC(=O)CCl)ccc3C2=O)C(=O)N1] |
| 1-30 | 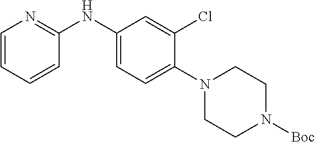 [SMILES: CC(C)(C)OC(=O)N1CCN(c2ccc(Nc3ccccn3)cc2Cl)CC1] | 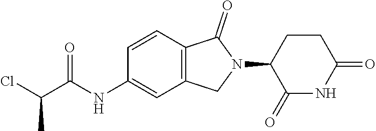 [SMILES: C[C@@H](Cl)C(=O)Nc1ccc2c(c1)CN([C@H]1CCC(=O)NC1=O)C2=O] |
| 1-31 | 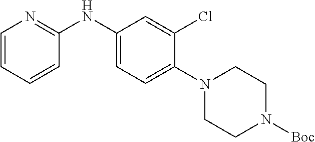 [SMILES: CC(C)(C)OC(=O)N1CCN(c2ccc(Nc3ccccn3)cc2Cl)CC1] | 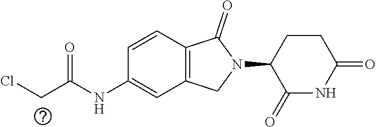 [SMILES: O=C1CC[C@H](N2Cc3cc(NC(=O)CCl)ccc3C2=O)C(=O)N1] |
| 1-32 | 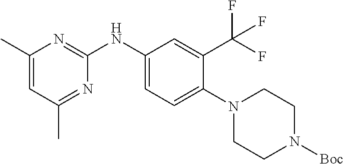 [SMILES: Cc1cc(C)nc(Nc2ccc(N3CCN(C(=O)OC(C)(C)C)CC3)c(C(F)(F)F)c2)n1] | 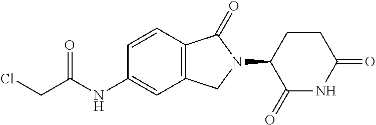 [SMILES: O=C1CC[C@H](N2Cc3cc(NC(=O)CCl)ccc3C2=O)C(=O)N1] |
| 1-33 | 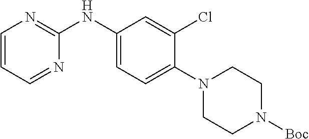 [SMILES: CC(C)(C)OC(=O)N1CCN(c2ccc(Nc3ncccn3)cc2Cl)CC1] | 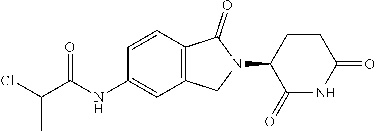 [SMILES: CC(Cl)C(=O)Nc1ccc2c(c1)CN([C@H]1CCC(=O)NC1=O)C2=O] |
| 1-34 | 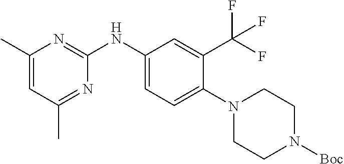 [SMILES: Cc1cc(C)nc(Nc2ccc(N3CCN(C(=O)OC(C)(C)C)CC3)c(C(F)(F)F)c2)n1] | 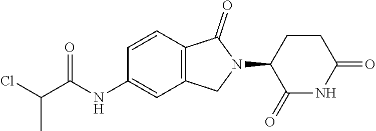 [SMILES: CC(Cl)C(=O)Nc1ccc2c(c1)CN([C@H]1CCC(=O)NC1=O)C2=O] |
| 1-35 | 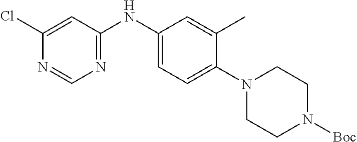 [SMILES: Cc1cc(Nc2cc(Cl)ncn2)ccc1N1CCN(C(=O)OC(C)(C)C)CC1] | 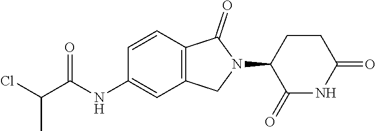 [SMILES: CC(Cl)C(=O)Nc1ccc2c(c1)CN([C@H]1CCC(=O)NC1=O)C2=O] |
| 1-36 | 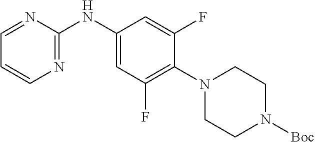 [SMILES: CC(C)(C)OC(=O)N1CCN(c2c(F)cc(Nc3ncccn3)cc2F)CC1] | 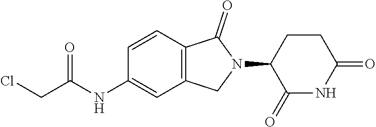 [SMILES: O=C1CC[C@H](N2Cc3cc(NC(=O)CCl)ccc3C2=O)C(=O)N1] |
| 1-37 | 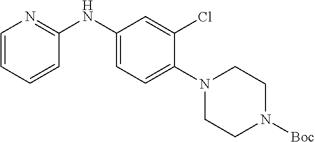 [SMILES: CC(C)(C)OC(=O)N1CCN(c2ccc(Nc3ccccn3)cc2Cl)CC1] | 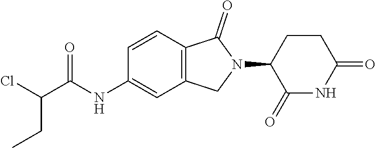 [SMILES: CCC(Cl)C(=O)Nc1ccc2c(c1)CN([C@H]1CCC(=O)NC1=O)C2=O] |
| 1-38 | 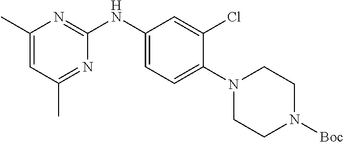 [SMILES: Cc1cc(C)nc(Nc2ccc(N3CCN(C(=O)OC(C)(C)C)CC3)c(Cl)c2)n1] | 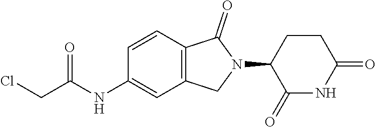 [SMILES: O=C1CC[C@H](N2Cc3cc(NC(=O)CCl)ccc3C2=O)C(=O)N1] |
| 1-39 | 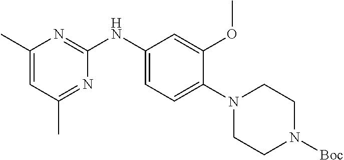 [SMILES: COc1cc(Nc2nc(C)cc(C)n2)ccc1N1CCN(C(=O)OC(C)(C)C)CC1] | 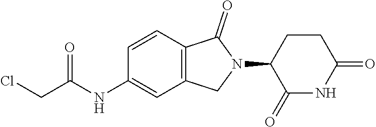 [SMILES: O=C1CC[C@H](N2Cc3cc(NC(=O)CCl)ccc3C2=O)C(=O)N1] |
| 1-40 | 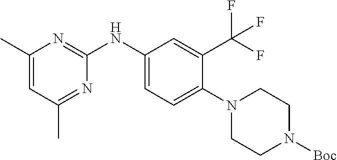 [SMILES: Cc1cc(C)nc(Nc2ccc(N3CCN(C(=O)OC(C)(C)C)CC3)c(C(F)(F)F)c2)n1] | 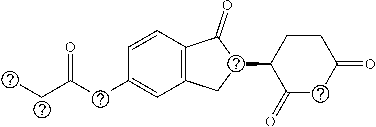 [SMILES: CCC(=O)Cc1ccc2c(c1)CC([C@H]1CCC(=O)CC1=O)C2=O] |
| 1-41 | 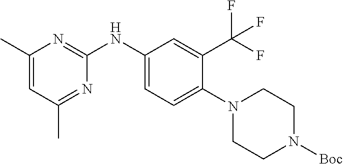 [SMILES: Cc1cc(C)nc(Nc2ccc(N3CCN(C(=O)OC(C)(C)C)CC3)c(C(F)(F)F)c2)n1] | 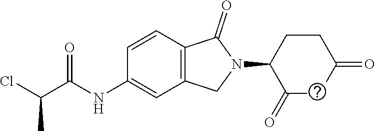 [SMILES: C[C@@H](Cl)C(=O)Nc1ccc2c(c1)CN([C@H]1CCC(=O)CC1=O)C2=O] |
| 1-42 | 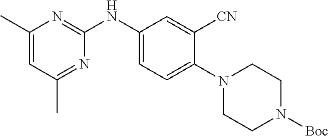 [SMILES: Cc1cc(C)nc(Nc2ccc(N3CCN(C(=O)OC(C)(C)C)CC3)c(C#N)c2)n1] | 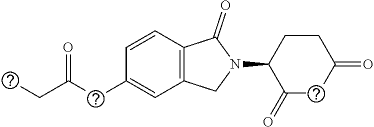 [SMILES: CCC(=O)Cc1ccc2c(c1)CN([C@H]1CCC(=O)CC1=O)C2=O] |
| 1-43 | 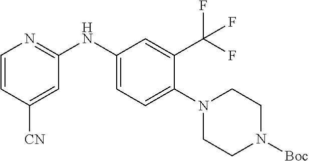 [SMILES: CC(C)(C)OC(=O)N1CCN(c2ccc(Nc3cc(C#N)ccn3)cc2C(F)(F)F)CC1] | 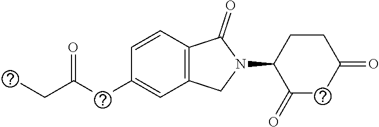 [SMILES: CCC(=O)Cc1ccc2c(c1)CN([C@H]1CCC(=O)CC1=O)C2=O] |
| 1-44 | 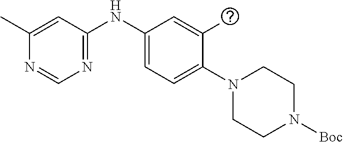 [SMILES: Cc1cc(Nc2ccc(N3CCN(C(=O)OC(C)(C)C)CC3)c(C)c2)ncn1] | 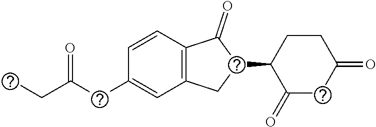 [SMILES: CCC(=O)Cc1ccc2c(c1)CN([C@H]1CCC(=O)CC1=O)C2=O] |
| 1-45 | 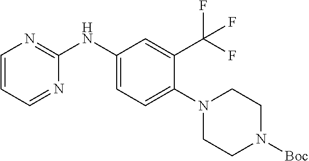 [SMILES: CC(C)(C)OC(=O)N1CCN(c2ccc(Nc3ncccn3)cc2C(F)(F)F)CC1] | 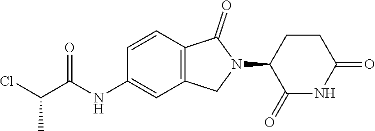 [SMILES: C[C@H](Cl)C(=O)Nc1ccc2c(c1)CN([C@H]1CCC(=O)NC1=O)C2=O] |
| 1-46 | 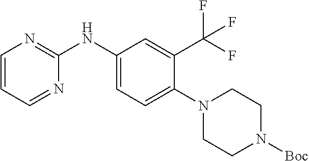 [SMILES: CC(C)(C)OC(=O)N1CCN(c2ccc(Nc3ncccn3)cc2C(F)(F)F)CC1] | 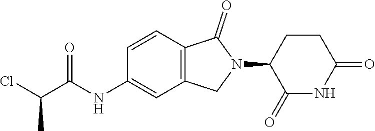 [SMILES: C[C@@H](Cl)C(=O)Nc1ccc2c(c1)CN([C@H]1CCC(=O)NC1=O)C2=O] |
| 1-47 | 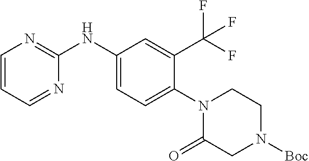 [SMILES: CC(C)(C)OC(=O)N1CCN(c2ccc(Nc3ncccn3)cc2C(F)(F)F)C(=O)C1] | 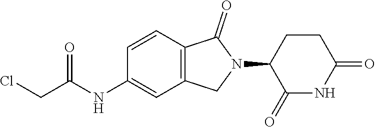 [SMILES: O=C1CC[C@H](N2Cc3cc(NC(=O)CCl)ccc3C2=O)C(=O)N1] |
| 1-48 | 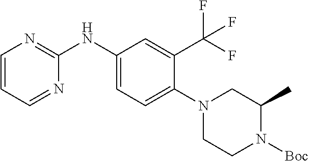 [SMILES: C[C@@H]1CN(c2ccc(Nc3ncccn3)cc2C(F)(F)F)CCN1C(=O)OC(C)(C)C] | 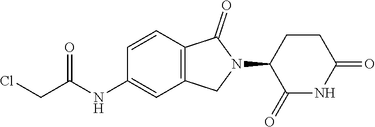 [SMILES: O=C1CC[C@H](N2Cc3cc(NC(=O)CCl)ccc3C2=O)C(=O)N1] |
| 1-49 | 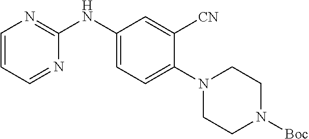 [SMILES: CC(C)(C)OC(=O)N1CCN(c2ccc(Nc3ncccn3)cc2C#N)CC1] | 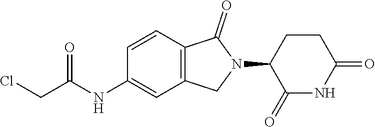 [SMILES: O=C1CC[C@H](N2Cc3cc(NC(=O)CCl)ccc3C2=O)C(=O)N1] |
| 1-50 | 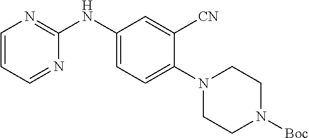 [SMILES: CC(C)(C)OC(=O)N1CCN(c2ccc(Nc3ncccn3)cc2C#N)CC1] | 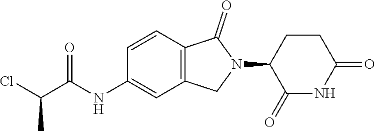 [SMILES: C[C@@H](Cl)C(=O)Nc1ccc2c(c1)CN([C@H]1CCC(=O)NC1=O)C2=O] |
| 1-51 | 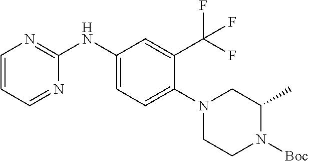 [SMILES: C[C@H]1CN(c2ccc(Nc3ncccn3)cc2C(F)(F)F)CCN1C(=O)OC(C)(C)C] | 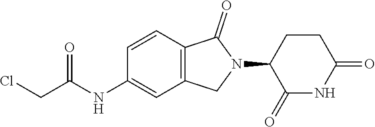 [SMILES: O=C1CC[C@H](N2Cc3cc(NC(=O)CCl)ccc3C2=O)C(=O)N1] |
| 1-52 | 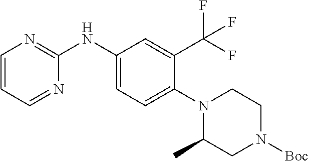 [SMILES: C[C@@H]1CN(C(=O)OC(C)(C)C)CCN1c1ccc(Nc2ncccn2)cc1C(F)(F)F] | 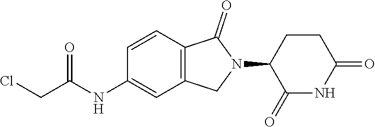 [SMILES: O=C1CC[C@H](N2Cc3cc(NC(=O)CCl)ccc3C2=O)C(=O)N1] |
| 1-53 | 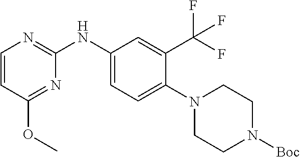 [SMILES: COc1ccnc(Nc2ccc(N3CCN(C(=O)OC(C)(C)C)CC3)c(C(F)(F)F)c2)n1] | 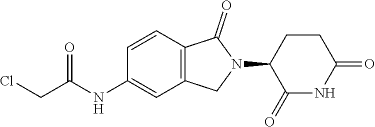 [SMILES: O=C1CC[C@H](N2Cc3cc(NC(=O)CCl)ccc3C2=O)C(=O)N1] |
| 1-54 | 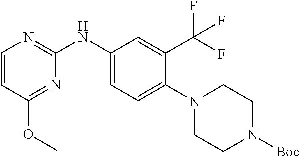 [SMILES: COc1ccnc(Nc2ccc(N3CCN(C(=O)OC(C)(C)C)CC3)c(C(F)(F)F)c2)n1] | 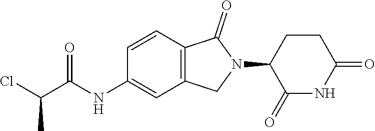 [SMILES: C[C@@H](Cl)C(=O)Nc1ccc2c(c1)CN([C@H]1CCC(=O)NC1=O)C2=O] |
| 1-55 | 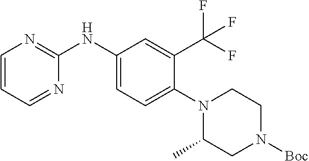 [SMILES: C[C@H]1CN(C(=O)OC(C)(C)C)CCN1c1ccc(Nc2ncccn2)cc1C(F)(F)F] | 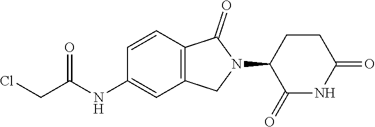 [SMILES: O=C1CC[C@H](N2Cc3cc(NC(=O)CCl)ccc3C2=O)C(=O)N1] |
| 1-56 | 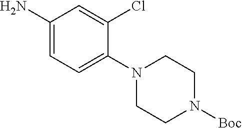 [SMILES: CC(C)(C)OC(=O)N1CCN(c2ccc(N)cc2Cl)CC1] | 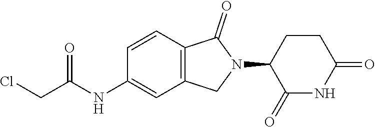 [SMILES: O=C1CC[C@H](N2Cc3cc(NC(=O)CCl)ccc3C2=O)C(=O)N1] |
| 1-57 | 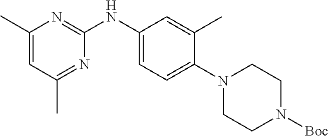 [SMILES: Cc1cc(C)nc(Nc2ccc(N3CCN(C(=O)OC(C)(C)C)CC3)c(C)c2)n1] | 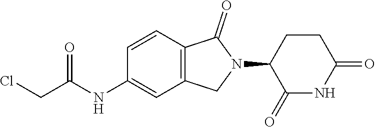 [SMILES: O=C1CC[C@H](N2Cc3cc(NC(=O)CCl)ccc3C2=O)C(=O)N1] |
| 1-58 | 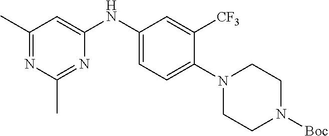 [SMILES: Cc1cc(Nc2ccc(N3CCN(C(=O)OC(C)(C)C)CC3)c(C(F)(F)F)c2)nc(C)n1] | 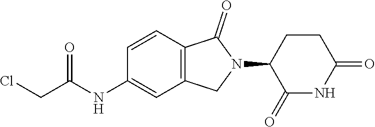 [SMILES: O=C1CC[C@H](N2Cc3cc(NC(=O)CCl)ccc3C2=O)C(=O)N1] |
| 1-59 | 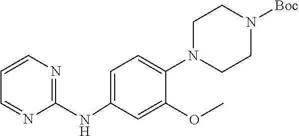 [SMILES: COc1cc(Nc2ncccn2)ccc1N1CCN(C(=O)OC(C)(C)C)CC1] | 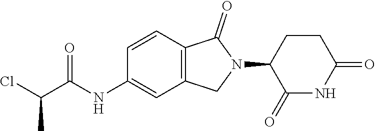 [SMILES: C[C@@H](Cl)C(=O)Nc1ccc2c(c1)CN([C@H]1CCC(=O)NC1=O)C2=O] |
| 1-60 | 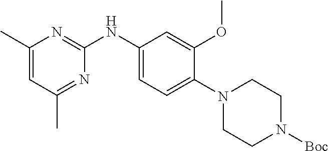 [SMILES: COc1cc(Nc2nc(C)cc(C)n2)ccc1N1CCN(C(=O)OC(C)(C)C)CC1] | 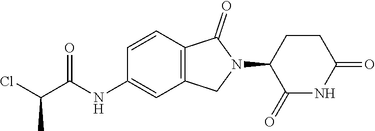 [SMILES: C[C@@H](Cl)C(=O)Nc1ccc2c(c1)CN([C@H]1CCC(=O)NC1=O)C2=O] |
| 1-61 | 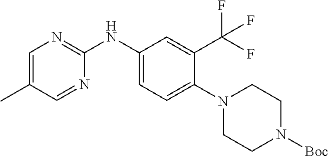 [SMILES: Cc1cnc(Nc2ccc(N3CCN(C(=O)OC(C)(C)C)CC3)c(C(F)(F)F)c2)nc1] | 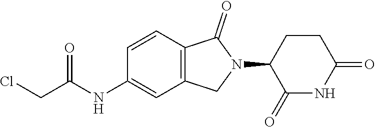 [SMILES: O=C1CC[C@H](N2Cc3cc(NC(=O)CCl)ccc3C2=O)C(=O)N1] |
| 1-62 | 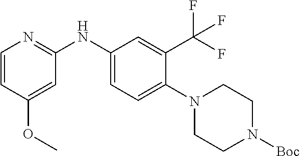 [SMILES: COc1ccnc(Nc2ccc(N3CCN(C(=O)OC(C)(C)C)CC3)c(C(F)(F)F)c2)c1] | 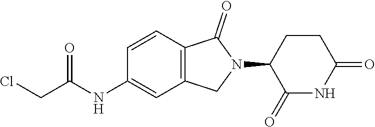 [SMILES: O=C1CC[C@H](N2Cc3cc(NC(=O)CCl)ccc3C2=O)C(=O)N1] |
| 1-63 | 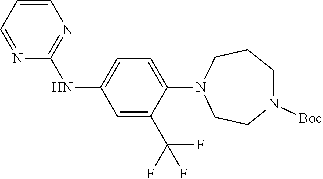 [SMILES: CC(C)(C)OC(=O)N1CCCN(c2ccc(Nc3ncccn3)cc2C(F)(F)F)CC1] | 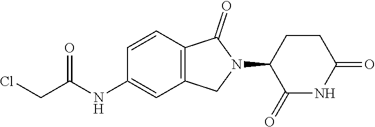 [SMILES: O=C1CC[C@H](N2Cc3cc(NC(=O)CCl)ccc3C2=O)C(=O)N1] |
| 1-64 | 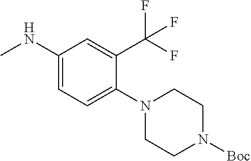 [SMILES: CNc1ccc(N2CCN(C(=O)OC(C)(C)C)CC2)c(C(F)(F)F)c1] | 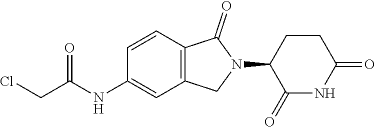 [SMILES: O=C1CC[C@H](N2Cc3cc(NC(=O)CCl)ccc3C2=O)C(=O)N1] |
| 1-65 | 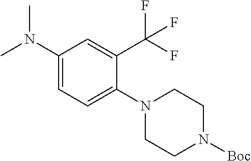 [SMILES: CN(C)c1ccc(N2CCN(C(=O)OC(C)(C)C)CC2)c(C(F)(F)F)c1] | 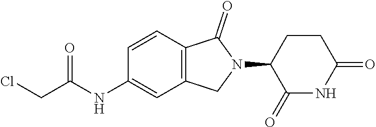 [SMILES: O=C1CC[C@H](N2Cc3cc(NC(=O)CCl)ccc3C2=O)C(=O)N1] |
| 1-66 | 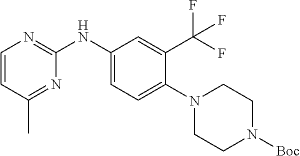 [SMILES: Cc1ccnc(Nc2ccc(N3CCN(C(=O)OC(C)(C)C)CC3)c(C(F)(F)F)c2)n1] | 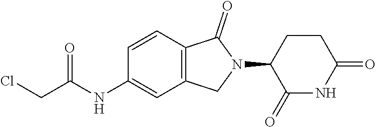 [SMILES: O=C1CC[C@H](N2Cc3cc(NC(=O)CCl)ccc3C2=O)C(=O)N1] |
| 1-67 | 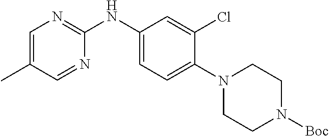 [SMILES: Cc1cnc(Nc2ccc(N3CCN(C(=O)OC(C)(C)C)CC3)c(Cl)c2)nc1] | 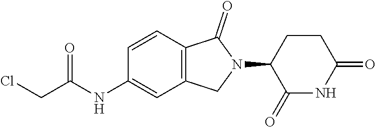 [SMILES: O=C1CC[C@H](N2Cc3cc(NC(=O)CCl)ccc3C2=O)C(=O)N1] |
| 1-68 | 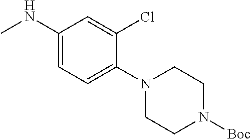 [SMILES: CNc1ccc(N2CCN(C(=O)OC(C)(C)C)CC2)c(Cl)c1] | 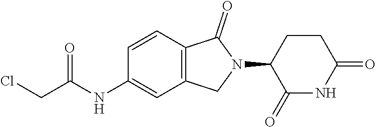 [SMILES: O=C1CC[C@H](N2Cc3cc(NC(=O)CCl)ccc3C2=O)C(=O)N1] |
| 1-69 | 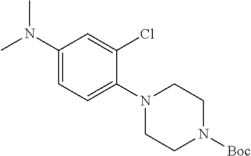 [SMILES: CN(C)c1ccc(N2CCN(C(=O)OC(C)(C)C)CC2)c(Cl)c1] | 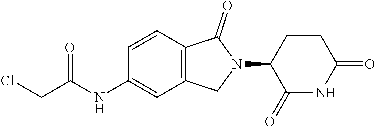 [SMILES: O=C1CC[C@H](N2Cc3cc(NC(=O)CCl)ccc3C2=O)C(=O)N1] |
| 1-70 | 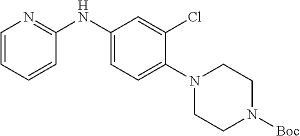 [SMILES: CC(C)(C)OC(=O)N1CCN(c2ccc(Nc3ccccn3)cc2Cl)CC1] | 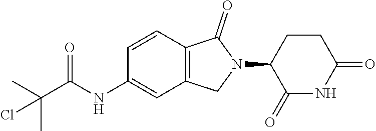 [SMILES: CC(C)(Cl)C(=O)Nc1ccc2c(c1)CN([C@H]1CCC(=O)NC1=O)C2=O] |
| 1-71 | 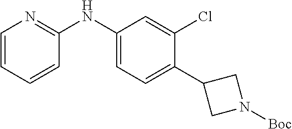 [SMILES: CC(C)(C)OC(=O)N1CC(c2ccc(Nc3ccccn3)cc2Cl)C1] | 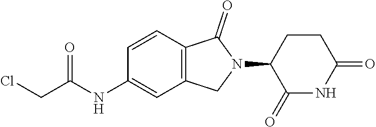 [SMILES: O=C1CC[C@H](N2Cc3cc(NC(=O)CCl)ccc3C2=O)C(=O)N1] |
| 1-72 | 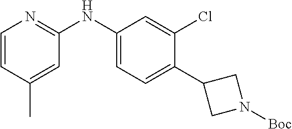 [SMILES: Cc1ccnc(Nc2ccc(C3CN(C(=O)OC(C)(C)C)C3)c(Cl)c2)c1] | 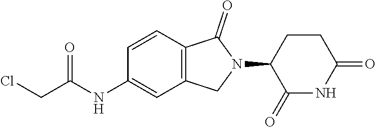 [SMILES: O=C1CC[C@H](N2Cc3cc(NC(=O)CCl)ccc3C2=O)C(=O)N1] |
| 1-73 | 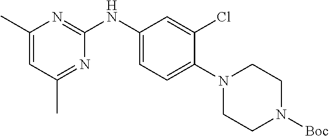 [SMILES: Cc1cc(C)nc(Nc2ccc(N3CCN(C(=O)OC(C)(C)C)CC3)c(Cl)c2)n1] | 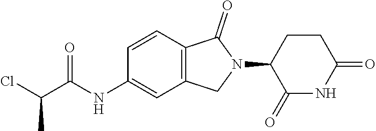 [SMILES: C[C@@H](Cl)C(=O)Nc1ccc2c(c1)CN([C@H]1CCC(=O)NC1=O)C2=O] |
| 1-74 | 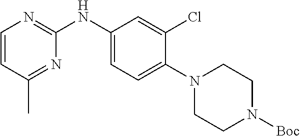 [SMILES: Cc1ccnc(Nc2ccc(N3CCN(C(=O)OC(C)(C)C)CC3)c(Cl)c2)n1] | 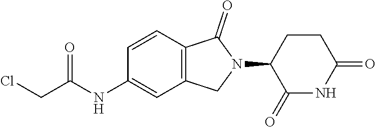 [SMILES: O=C1CC[C@H](N2Cc3cc(NC(=O)CCl)ccc3C2=O)C(=O)N1] |
| 1-75 | 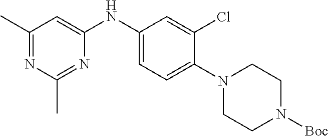 [SMILES: Cc1cc(Nc2ccc(N3CCN(C(=O)OC(C)(C)C)CC3)c(Cl)c2)nc(C)n1] | 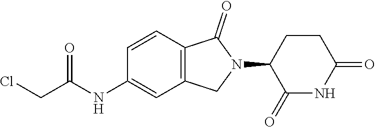 [SMILES: O=C1CC[C@H](N2Cc3cc(NC(=O)CCl)ccc3C2=O)C(=O)N1] |
| 1-76 | 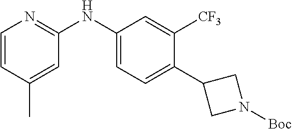 [SMILES: Cc1ccnc(Nc2ccc(C3CN(C(=O)OC(C)(C)C)C3)c(C(F)(F)F)c2)c1] | 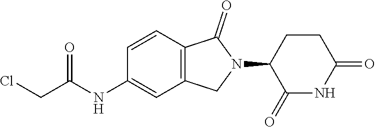 [SMILES: O=C1CC[C@H](N2Cc3cc(NC(=O)CCl)ccc3C2=O)C(=O)N1] |
| 1-77 | 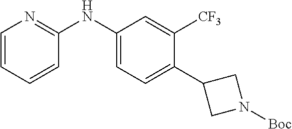 [SMILES: CC(C)(C)OC(=O)N1CC(c2ccc(Nc3ccccn3)cc2C(F)(F)F)C1] | 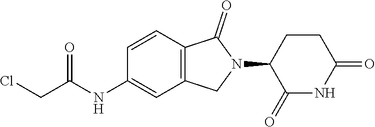 [SMILES: O=C1CC[C@H](N2Cc3cc(NC(=O)CCl)ccc3C2=O)C(=O)N1] |
| 1-78 | 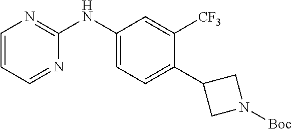 [SMILES: CC(C)(C)OC(=O)N1CC(c2ccc(Nc3ncccn3)cc2C(F)(F)F)C1] | 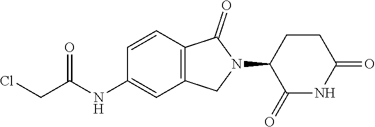 [SMILES: O=C1CC[C@H](N2Cc3cc(NC(=O)CCl)ccc3C2=O)C(=O)N1] |
| 1-79 | 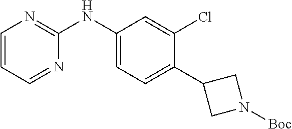 [SMILES: CC(C)(C)OC(=O)N1CC(c2ccc(Nc3ncccn3)cc2Cl)C1] | 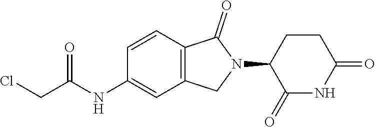 [SMILES: O=C1CC[C@H](N2Cc3cc(NC(=O)CCl)ccc3C2=O)C(=O)N1] |
| 1-80 | 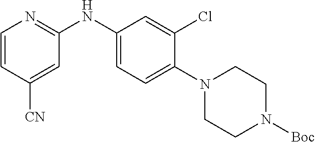 [SMILES: CC(C)(C)OC(=O)N1CCN(c2ccc(Nc3cc(C#N)ccn3)cc2Cl)CC1] | 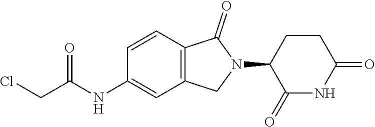 [SMILES: O=C1CC[C@H](N2Cc3cc(NC(=O)CCl)ccc3C2=O)C(=O)N1] |
| 1-81 | 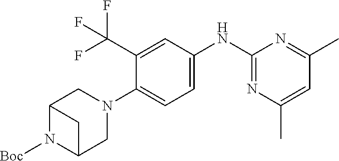 [SMILES: Cc1cc(C)nc(Nc2ccc(N3CC4CC(C3)N4C(=O)OC(C)(C)C)c(C(F)(F)F)c2)n1] | 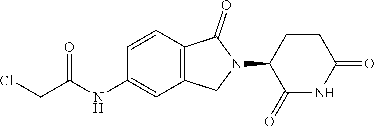 [SMILES: O=C1CC[C@H](N2Cc3cc(NC(=O)CCl)ccc3C2=O)C(=O)N1] |
| 1-82 | 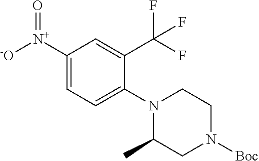 | 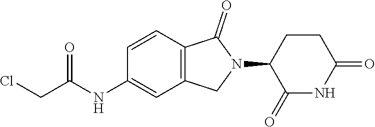 [SMILES: O=C1CC[C@H](N2Cc3cc(NC(=O)CCl)ccc3C2=O)C(=O)N1] |
| 1-83 | 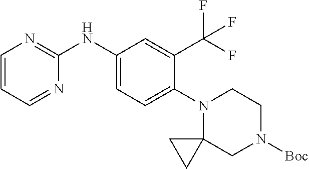 [SMILES: CC(C)(C)OC(=O)N1CCN(c2ccc(Nc3ncccn3)cc2C(F)(F)F)C2(CC2)C1] | 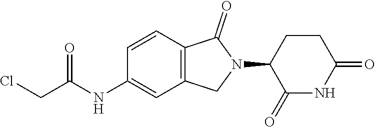 [SMILES: O=C1CC[C@H](N2Cc3cc(NC(=O)CCl)ccc3C2=O)C(=O)N1] |
| 1-84 | 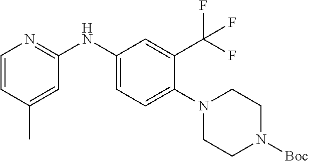 [SMILES: Cc1ccnc(Nc2ccc(N3CCN(C(=O)OC(C)(C)C)CC3)c(C(F)(F)F)c2)c1] | 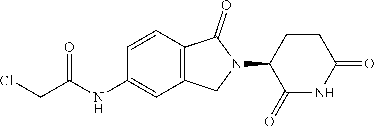 [SMILES: O=C1CC[C@H](N2Cc3cc(NC(=O)CCl)ccc3C2=O)C(=O)N1] |
| 1-85 | 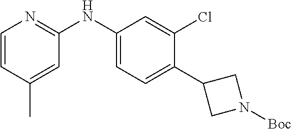 [SMILES: Cc1ccnc(Nc2ccc(C3CN(C(=O)OC(C)(C)C)C3)c(Cl)c2)c1] | 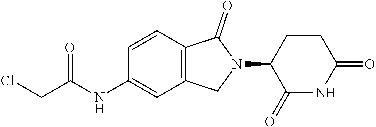 [SMILES: O=C1CC[C@H](N2Cc3cc(NC(=O)CCl)ccc3C2=O)C(=O)N1] |
| 1-86 | 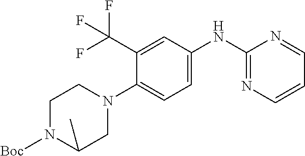 | 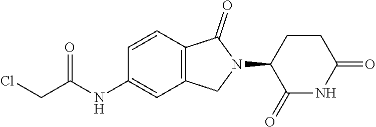 [SMILES: O=C1CC[C@H](N2Cc3cc(NC(=O)CCl)ccc3C2=O)C(=O)N1] |
| 1-87 | 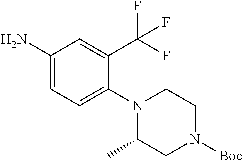 [SMILES: C[C@H]1CN(C(=O)OC(C)(C)C)CCN1c1ccc(N)cc1C(F)(F)F] | 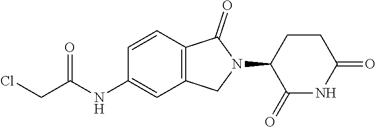 [SMILES: O=C1CC[C@H](N2Cc3cc(NC(=O)CCl)ccc3C2=O)C(=O)N1] |
| 1-88 | 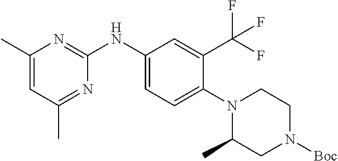 [SMILES: Cc1cc(C)nc(Nc2ccc(N3CCN(C(=O)OC(C)(C)C)C[C@H]3C)c(C(F)(F)F)c2)n1] | 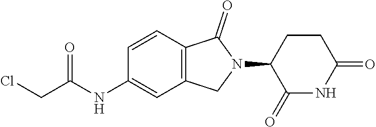 [SMILES: O=C1CC[C@H](N2Cc3cc(NC(=O)CCl)ccc3C2=O)C(=O)N1] |
| 1-89 | 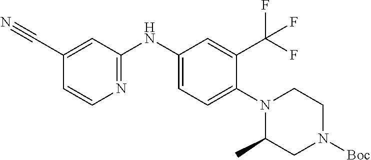 [SMILES: C[C@@H]1CN(C(=O)OC(C)(C)C)CCN1c1ccc(Nc2cc(C#N)ccn2)cc1C(F)(F)F] | 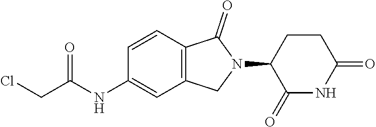 [SMILES: O=C1CC[C@H](N2Cc3cc(NC(=O)CCl)ccc3C2=O)C(=O)N1] |
| 1-90 | 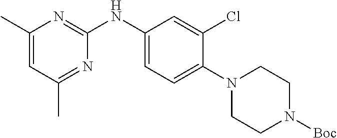 [SMILES: Cc1cc(C)nc(Nc2ccc(N3CCN(C(=O)OC(C)(C)C)CC3)c(Cl)c2)n1] | 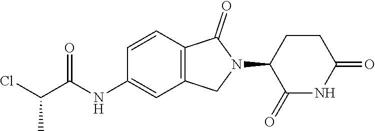 [SMILES: C[C@H](Cl)C(=O)Nc1ccc2c(c1)CN([C@H]1CCC(=O)NC1=O)C2=O] |
| 1-91 | 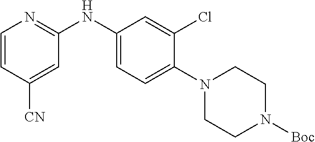 [SMILES: CC(C)(C)OC(=O)N1CCN(c2ccc(Nc3cc(C#N)ccn3)cc2Cl)CC1] | 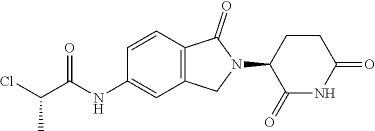 [SMILES: C[C@H](Cl)C(=O)Nc1ccc2c(c1)CN([C@H]1CCC(=O)NC1=O)C2=O] |
| 1-92 | 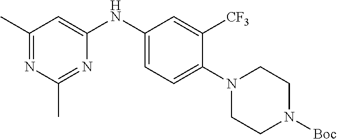 [SMILES: Cc1cc(Nc2ccc(N3CCN(C(=O)OC(C)(C)C)CC3)c(C(F)(F)F)c2)nc(C)n1] | 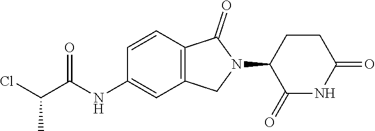 [SMILES: C[C@H](Cl)C(=O)Nc1ccc2c(c1)CN([C@H]1CCC(=O)NC1=O)C2=O] |
| 1-93 | 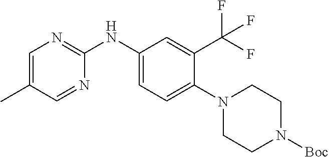 [SMILES: Cc1cnc(Nc2ccc(N3CCN(C(=O)OC(C)(C)C)CC3)c(C(F)(F)F)c2)nc1] | 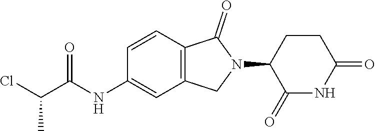 [SMILES: C[C@H](Cl)C(=O)Nc1ccc2c(c1)CN([C@H]1CCC(=O)NC1=O)C2=O] |
| 1-94 | 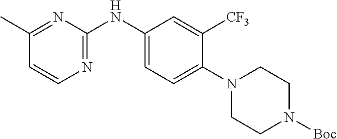 [SMILES: Cc1ccnc(Nc2ccc(N3CCN(C(=O)OC(C)(C)C)CC3)c(C(F)(F)F)c2)n1] | 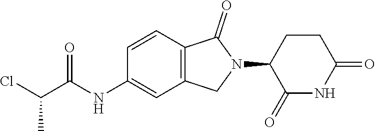 [SMILES: C[C@H](Cl)C(=O)Nc1ccc2c(c1)CN([C@H]1CCC(=O)NC1=O)C2=O] |
| 1-95 | 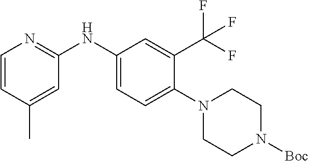 [SMILES: Cc1ccnc(Nc2ccc(N3CCN(C(=O)OC(C)(C)C)CC3)c(C(F)(F)F)c2)c1] | 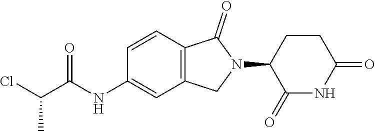 [SMILES: C[C@H](Cl)C(=O)Nc1ccc2c(c1)CN([C@H]1CCC(=O)NC1=O)C2=O] |
| 1-96 | 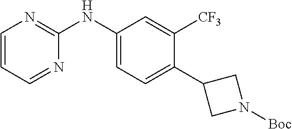 [SMILES: CC(C)(C)OC(=O)N1CC(c2ccc(Nc3ncccn3)cc2C(F)(F)F)C1] | 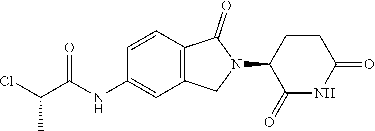 [SMILES: C[C@H](Cl)C(=O)Nc1ccc2c(c1)CN([C@H]1CCC(=O)NC1=O)C2=O] |
| 1-97 | 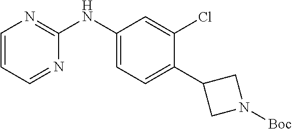 [SMILES: CC(C)(C)OC(=O)N1CC(c2ccc(Nc3ncccn3)cc2Cl)C1] | 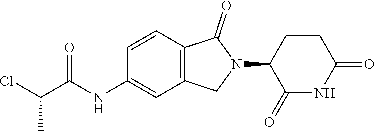 [SMILES: C[C@H](Cl)C(=O)Nc1ccc2c(c1)CN([C@H]1CCC(=O)NC1=O)C2=O] |
| 1-98 | 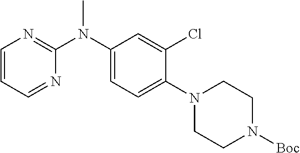 [SMILES: CN(c1ccc(N2CCN(C(=O)OC(C)(C)C)CC2)c(Cl)c1)c1ncccn1] | 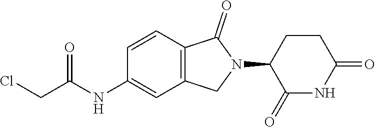 [SMILES: O=C1CC[C@H](N2Cc3cc(NC(=O)CCl)ccc3C2=O)C(=O)N1] |
| 1-99 | 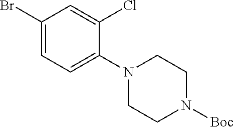 [SMILES: CC(C)(C)OC(=O)N1CCN(c2ccc(Br)cc2Cl)CC1] | 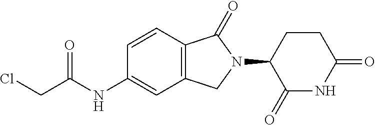 [SMILES: O=C1CC[C@H](N2Cc3cc(NC(=O)CCl)ccc3C2=O)C(=O)N1] |
| 1-100 | 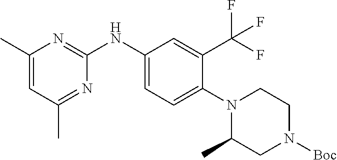 [SMILES: Cc1cc(C)nc(Nc2ccc(N3CCN(C(=O)OC(C)(C)C)C[C@H]3C)c(C(F)(F)F)c2)n1] | 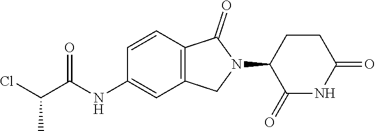 [SMILES: C[C@H](Cl)C(=O)Nc1ccc2c(c1)CN([C@H]1CCC(=O)NC1=O)C2=O] |
| 1-101 | 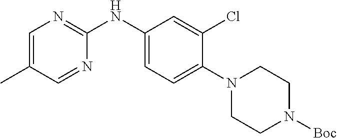 [SMILES: Cc1cnc(Nc2ccc(N3CCN(C(=O)OC(C)(C)C)CC3)c(Cl)c2)nc1] | 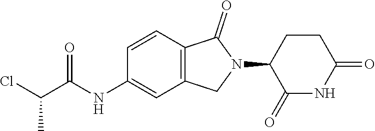 [SMILES: C[C@H](Cl)C(=O)Nc1ccc2c(c1)CN([C@H]1CCC(=O)NC1=O)C2=O] |
| 1-102 | 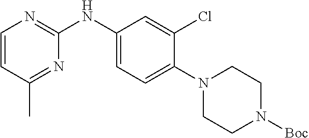 [SMILES: Cc1ccnc(Nc2ccc(N3CCN(C(=O)OC(C)(C)C)CC3)c(Cl)c2)n1] | 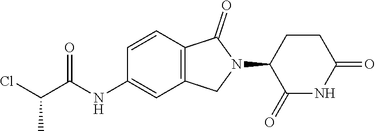 [SMILES: C[C@H](Cl)C(=O)Nc1ccc2c(c1)CN([C@H]1CCC(=O)NC1=O)C2=O] |
| 1-103 | 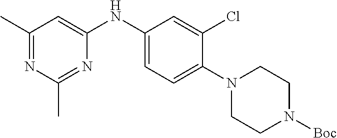 [SMILES: Cc1cc(Nc2ccc(N3CCN(C(=O)OC(C)(C)C)CC3)c(Cl)c2)nc(C)n1] | 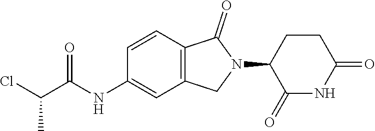 [SMILES: C[C@H](Cl)C(=O)Nc1ccc2c(c1)CN([C@H]1CCC(=O)NC1=O)C2=O] |
| 1-104 | 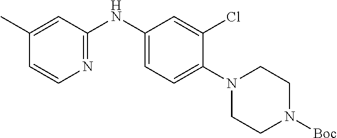 [SMILES: Cc1ccnc(Nc2ccc(N3CCN(C(=O)OC(C)(C)C)CC3)c(Cl)c2)c1] | 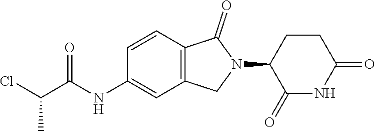 [SMILES: C[C@H](Cl)C(=O)Nc1ccc2c(c1)CN([C@H]1CCC(=O)NC1=O)C2=O] |
| 1-105 | 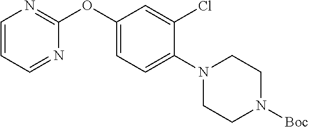 [SMILES: CC(C)(C)OC(=O)N1CCN(c2ccc(Oc3ncccn3)cc2Cl)CC1] | 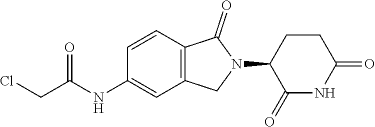 [SMILES: O=C1CC[C@H](N2Cc3cc(NC(=O)CCl)ccc3C2=O)C(=O)N1] |
| 1-106 | 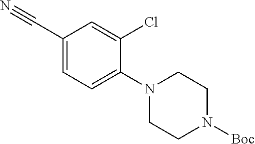 [SMILES: CC(C)(C)OC(=O)N1CCN(c2ccc(C#N)cc2Cl)CC1] | 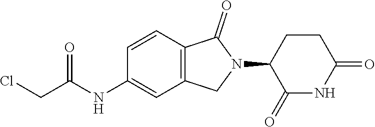 [SMILES: O=C1CC[C@H](N2Cc3cc(NC(=O)CCl)ccc3C2=O)C(=O)N1] |
| 1-107 | 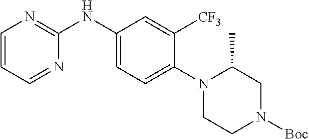 [SMILES: C[C@@H]1CN(C(=O)OC(C)(C)C)CCN1c1ccc(Nc2ncccn2)cc1C(F)(F)F] | 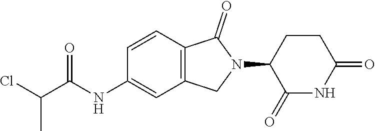 [SMILES: CC(Cl)C(=O)Nc1ccc2c(c1)CN([C@H]1CCC(=O)NC1=O)C2=O] |
| 1-108 | 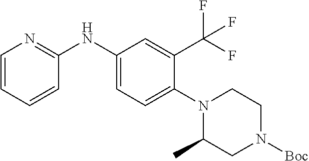 [SMILES: C[C@@H]1CN(C(=O)OC(C)(C)C)CCN1c1ccc(Nc2ccccn2)cc1C(F)(F)F] | 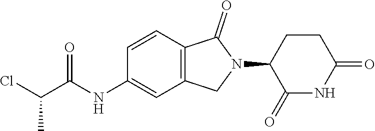 [SMILES: C[C@H](Cl)C(=O)Nc1ccc2c(c1)CN([C@H]1CCC(=O)NC1=O)C2=O] |
| 1-109 | 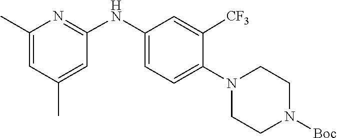 [SMILES: Cc1cc(C)nc(Nc2ccc(N3CCN(C(=O)OC(C)(C)C)CC3)c(C(F)(F)F)c2)c1] | 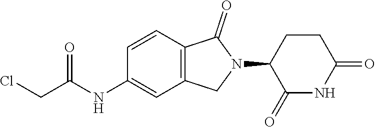 [SMILES: O=C1CC[C@H](N2Cc3cc(NC(=O)CCl)ccc3C2=O)C(=O)N1] |
| 1-110 | 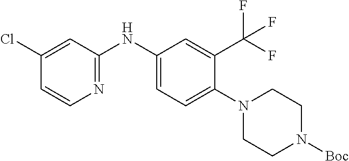 [SMILES: CC(C)(C)OC(=O)N1CCN(c2ccc(Nc3cc(Cl)ccn3)cc2C(F)(F)F)CC1] | 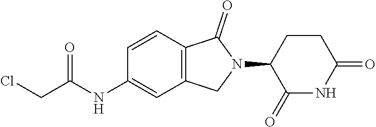 [SMILES: O=C1CC[C@H](N2Cc3cc(NC(=O)CCl)ccc3C2=O)C(=O)N1] |
| 1-111 | 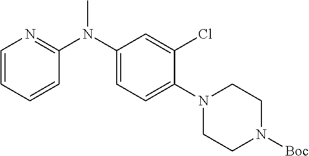 [SMILES: CN(c1ccc(N2CCN(C(=O)OC(C)(C)C)CC2)c(Cl)c1)c1ccccn1] | 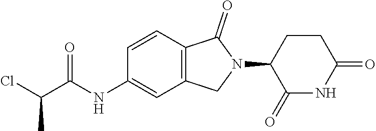 [SMILES: C[C@@H](Cl)C(=O)Nc1ccc2c(c1)CN([C@H]1CCC(=O)NC1=O)C2=O] |
| 1-112 | 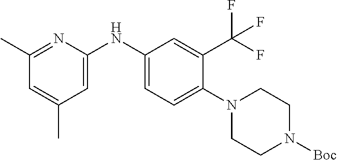 [SMILES: Cc1cc(C)nc(Nc2ccc(N3CCN(C(=O)OC(C)(C)C)CC3)c(C(F)(F)F)c2)c1] | 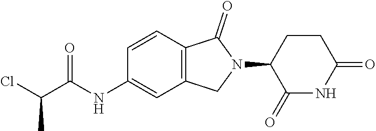 [SMILES: C[C@@H](Cl)C(=O)Nc1ccc2c(c1)CN([C@H]1CCC(=O)NC1=O)C2=O] |
| 1-113 | 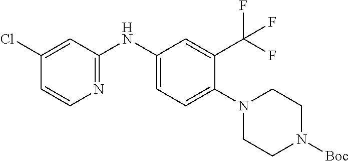 [SMILES: CC(C)(C)OC(=O)N1CCN(c2ccc(Nc3cc(Cl)ccn3)cc2C(F)(F)F)CC1] | 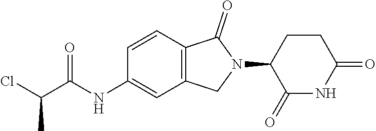 [SMILES: C[C@@H](Cl)C(=O)Nc1ccc2c(c1)CN([C@H]1CCC(=O)NC1=O)C2=O] |
| 1-114 | 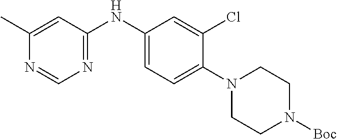 [SMILES: Cc1cc(Nc2ccc(N3CCN(C(=O)OC(C)(C)C)CC3)c(Cl)c2)ncn1] | 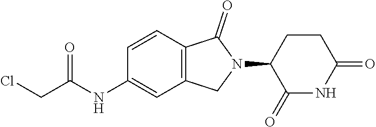 [SMILES: O=C1CC[C@H](N2Cc3cc(NC(=O)CCl)ccc3C2=O)C(=O)N1] |
| 1-115 | 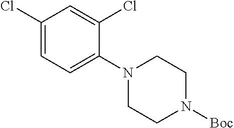 [SMILES: CC(C)(C)OC(=O)N1CCN(c2ccc(Cl)cc2Cl)CC1] | 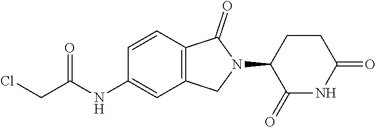 [SMILES: O=C1CC[C@H](N2Cc3cc(NC(=O)CCl)ccc3C2=O)C(=O)N1] |
| 1-116 | 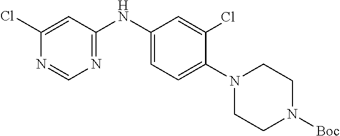 [SMILES: CC(C)(C)OC(=O)N1CCN(c2ccc(Nc3cc(Cl)ncn3)cc2Cl)CC1] | 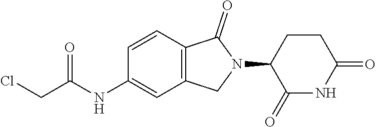 [SMILES: O=C1CC[C@H](N2Cc3cc(NC(=O)CCl)ccc3C2=O)C(=O)N1] |
| 1-117 | 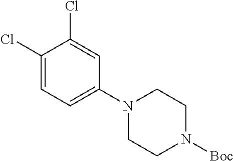 [SMILES: CC(C)(C)OC(=O)N1CCN(c2ccc(Cl)c(Cl)c2)CC1] | 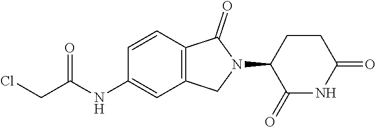 [SMILES: O=C1CC[C@H](N2Cc3cc(NC(=O)CCl)ccc3C2=O)C(=O)N1] |
| 1-118 | 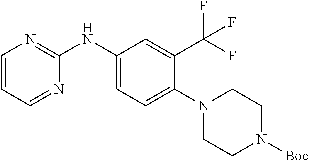 [SMILES: CC(C)(C)OC(=O)N1CCN(c2ccc(Nc3ncccn3)cc2C(F)(F)F)CC1] | 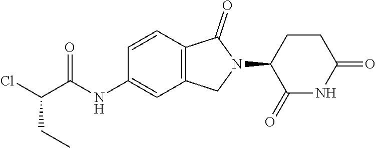 [SMILES: CC[C@H](Cl)C(=O)Nc1ccc2c(c1)CN([C@H]1CCC(=O)NC1=O)C2=O] |
| 1-119 | 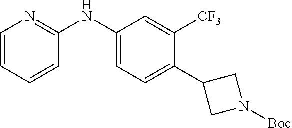 [SMILES: CC(C)(C)OC(=O)N1CC(c2ccc(Nc3ccccn3)cc2C(F)(F)F)C1] | 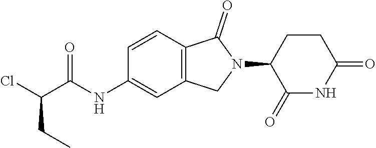 [SMILES: CC[C@@H](Cl)C(=O)Nc1ccc2c(c1)CN([C@H]1CCC(=O)NC1=O)C2=O] |
| 1-120 | 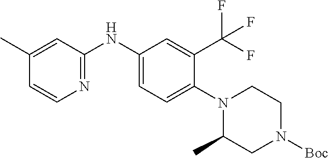 [SMILES: Cc1ccnc(Nc2ccc(N3CCN(C(=O)OC(C)(C)C)C[C@H]3C)c(C(F)(F)F)c2)c1] | 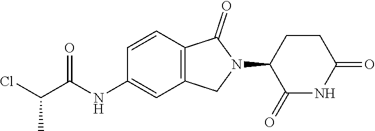 [SMILES: C[C@H](Cl)C(=O)Nc1ccc2c(c1)CN([C@H]1CCC(=O)NC1=O)C2=O] |
| 1-121 | 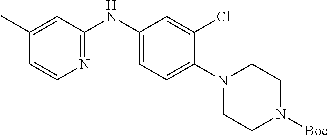 [SMILES: Cc1ccnc(Nc2ccc(N3CCN(C(=O)OC(C)(C)C)CC3)c(Cl)c2)c1] | 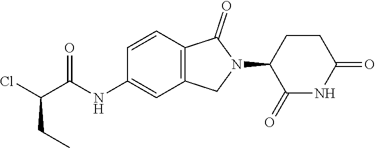 [SMILES: CC[C@@H](Cl)C(=O)Nc1ccc2c(c1)CN([C@H]1CCC(=O)NC1=O)C2=O] |
| 1-122 | 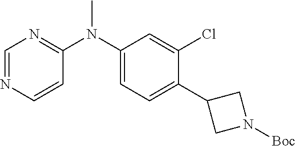 [SMILES: CN(c1ccc(C2CN(C(=O)OC(C)(C)C)C2)c(Cl)c1)c1ccncn1] | 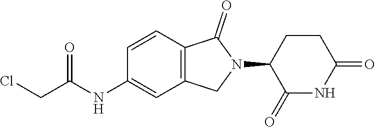 [SMILES: O=C1CC[C@H](N2Cc3cc(NC(=O)CCl)ccc3C2=O)C(=O)N1] |
| 1-123 | 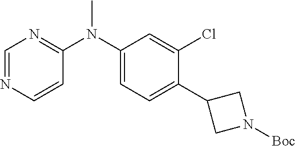 [SMILES: CN(c1ccc(C2CN(C(=O)OC(C)(C)C)C2)c(Cl)c1)c1ccncn1] | 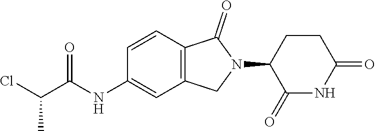 [SMILES: C[C@H](Cl)C(=O)Nc1ccc2c(c1)CN([C@H]1CCC(=O)NC1=O)C2=O] |
| 1-124 | 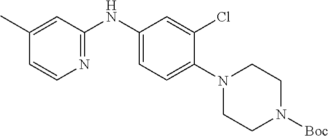 [SMILES: Cc1ccnc(Nc2ccc(N3CCN(C(=O)OC(C)(C)C)CC3)c(Cl)c2)c1] | 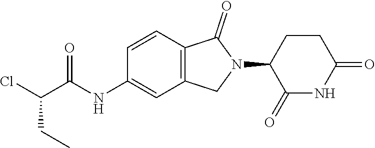 [SMILES: CC[C@H](Cl)C(=O)Nc1ccc2c(c1)CN([C@H]1CCC(=O)NC1=O)C2=O] |
| 1-125 | 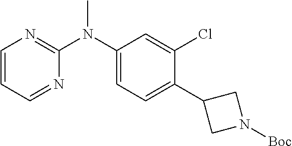 [SMILES: CN(c1ccc(C2CN(C(=O)OC(C)(C)C)C2)c(Cl)c1)c1ncccn1] | 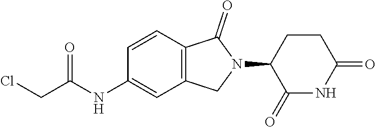 [SMILES: O=C1CC[C@H](N2Cc3cc(NC(=O)CCl)ccc3C2=O)C(=O)N1] |
| 1-126 | 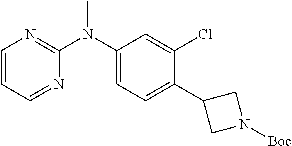 [SMILES: CN(c1ccc(C2CN(C(=O)OC(C)(C)C)C2)c(Cl)c1)c1ncccn1] | 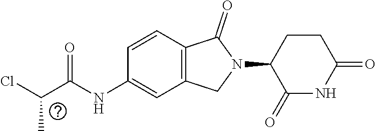 [SMILES: C[C@H](Cl)C(=O)Nc1ccc2c(c1)CN([C@H]1CCC(=O)NC1=O)C2=O] |
| 1-127 | 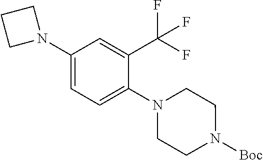 [SMILES: CC(C)(C)OC(=O)N1CCN(c2ccc(N3CCC3)cc2C(F)(F)F)CC1] | 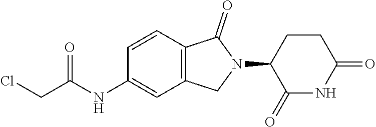 [SMILES: O=C1CC[C@H](N2Cc3cc(NC(=O)CCl)ccc3C2=O)C(=O)N1] |
| 1-128 | 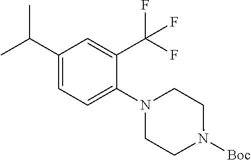 [SMILES: CC(C)c1ccc(N2CCN(C(=O)OC(C)(C)C)CC2)c(C(F)(F)F)c1] | 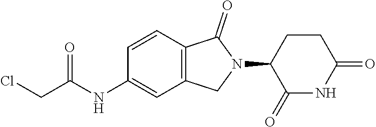 [SMILES: O=C1CC[C@H](N2Cc3cc(NC(=O)CCl)ccc3C2=O)C(=O)N1] |
| 1-129 | 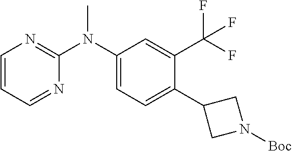 [SMILES: CN(c1ccc(C2CN(C(=O)OC(C)(C)C)C2)c(C(F)(F)F)c1)c1ncccn1] | 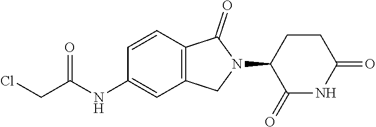 [SMILES: O=C1CC[C@H](N2Cc3cc(NC(=O)CCl)ccc3C2=O)C(=O)N1] |
| 1-130 | 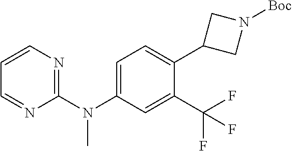 [SMILES: CN(c1ccc(C2CN(C(=O)OC(C)(C)C)C2)c(C(F)(F)F)c1)c1ncccn1] | 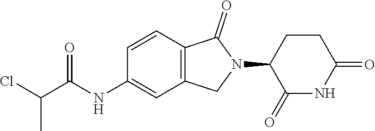 [SMILES: CC(Cl)C(=O)Nc1ccc2c(c1)CN([C@H]1CCC(=O)NC1=O)C2=O] |
| 1-131 | 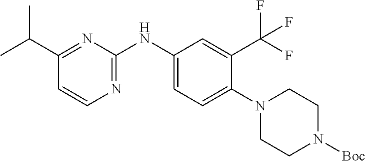 [SMILES: CC(C)c1ccnc(Nc2ccc(N3CCN(C(=O)OC(C)(C)C)CC3)c(C(F)(F)F)c2)n1] | 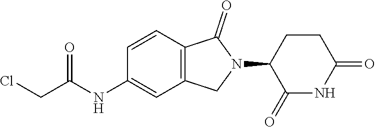 [SMILES: O=C1CC[C@H](N2Cc3cc(NC(=O)CCl)ccc3C2=O)C(=O)N1] |
| 1-132 | 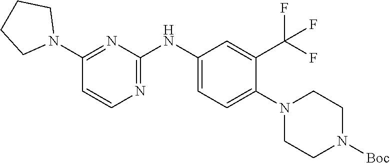 [SMILES: CC(C)(C)OC(=O)N1CCN(c2ccc(Nc3nccc(N4CCCC4)n3)cc2C(F)(F)F)CC1] | 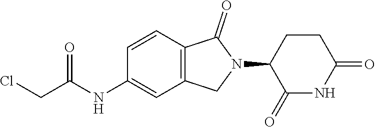 [SMILES: O=C1CC[C@H](N2Cc3cc(NC(=O)CCl)ccc3C2=O)C(=O)N1] |
| 1-133 | 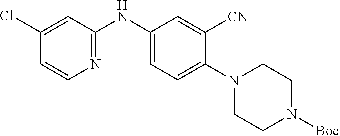 [SMILES: CC(C)(C)OC(=O)N1CCN(c2ccc(Nc3cc(Cl)ccn3)cc2C#N)CC1] | 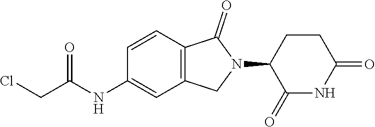 [SMILES: O=C1CC[C@H](N2Cc3cc(NC(=O)CCl)ccc3C2=O)C(=O)N1] |
| 1-134 | 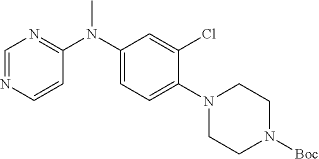 [SMILES: CN(c1ccc(N2CCN(C(=O)OC(C)(C)C)CC2)c(Cl)c1)c1ccncn1] | 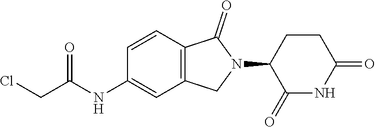 [SMILES: O=C1CC[C@H](N2Cc3cc(NC(=O)CCl)ccc3C2=O)C(=O)N1] |
| 1-135 | 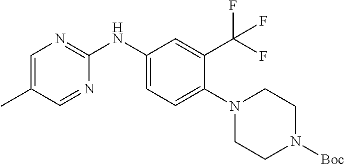 [SMILES: Cc1cnc(Nc2ccc(N3CCN(C(=O)OC(C)(C)C)CC3)c(C(F)(F)F)c2)nc1] | 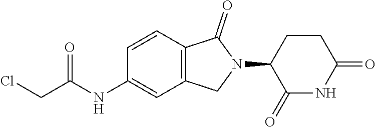 [SMILES: O=C1CC[C@H](N2Cc3cc(NC(=O)CCl)ccc3C2=O)C(=O)N1] |
| 1-136 | 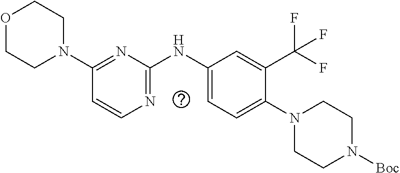 [SMILES: CC(C)(C)OC(=O)N1CCN(c2ccc(Nc3nccc(N4CCOCC4)n3)cc2C(F)(F)F)CC1] | 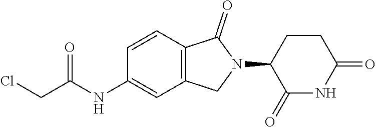 [SMILES: O=C1CC[C@H](N2Cc3cc(NC(=O)CCl)ccc3C2=O)C(=O)N1] |
| 1-137 | 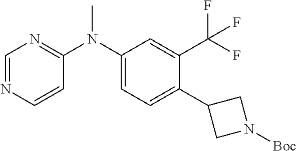 [SMILES: CN(c1ccc(C2CN(C(=O)OC(C)(C)C)C2)c(C(F)(F)F)c1)c1ccncn1] | 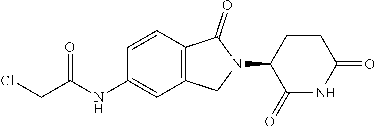 [SMILES: O=C1CC[C@H](N2Cc3cc(NC(=O)CCl)ccc3C2=O)C(=O)N1] |
| 1-138 | 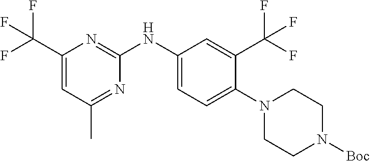 [SMILES: Cc1cc(C(F)(F)F)nc(Nc2ccc(N3CCN(C(=O)OC(C)(C)C)CC3)c(C(F)(F)F)c2)n1] | 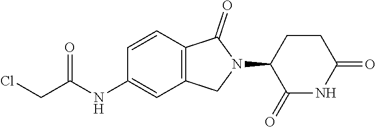 [SMILES: O=C1CC[C@H](N2Cc3cc(NC(=O)CCl)ccc3C2=O)C(=O)N1] |
| 1-139 | 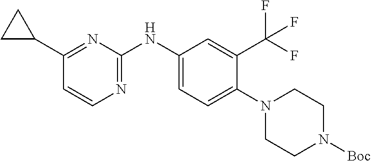 [SMILES: CC(C)(C)OC(=O)N1CCN(c2ccc(Nc3nccc(C4CC4)n3)cc2C(F)(F)F)CC1] | 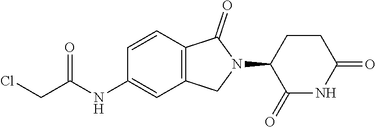 [SMILES: O=C1CC[C@H](N2Cc3cc(NC(=O)CCl)ccc3C2=O)C(=O)N1] |
| 1-140 | 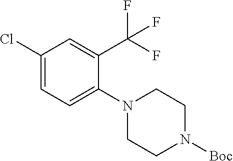 [SMILES: CC(C)(C)OC(=O)N1CCN(c2ccc(Cl)cc2C(F)(F)F)CC1] | 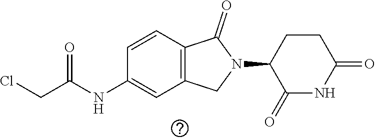 [SMILES: O=C1CC[C@H](N2Cc3cc(NC(=O)CCl)ccc3C2=O)C(=O)N1] |
| 1-141 | 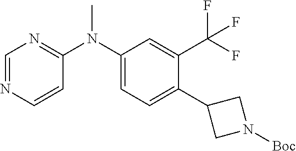 [SMILES: CN(c1ccc(C2CN(C(=O)OC(C)(C)C)C2)c(C(F)(F)F)c1)c1ccncn1] | 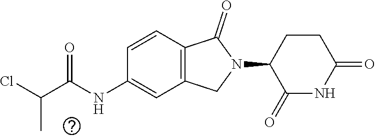 [SMILES: CC(Cl)C(=O)Nc1ccc2c(c1)CN([C@H]1CCC(=O)NC1=O)C2=O] |
| 1-142 | 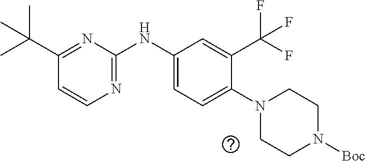 [SMILES: CC(C)(C)OC(=O)N1CCN(c2ccc(Nc3nccc(C(C)(C)C)n3)cc2C(F)(F)F)CC1] | 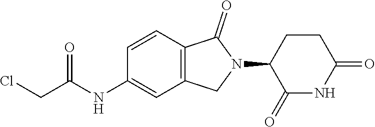 [SMILES: O=C1CC[C@H](N2Cc3cc(NC(=O)CCl)ccc3C2=O)C(=O)N1] |
| 1-143 | 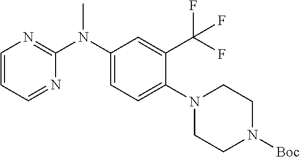 [SMILES: CN(c1ccc(N2CCN(C(=O)OC(C)(C)C)CC2)c(C(F)(F)F)c1)c1ncccn1] | 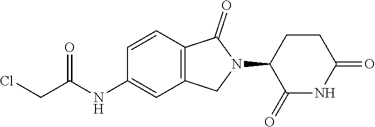 [SMILES: O=C1CC[C@H](N2Cc3cc(NC(=O)CCl)ccc3C2=O)C(=O)N1] |
| 1-144 | 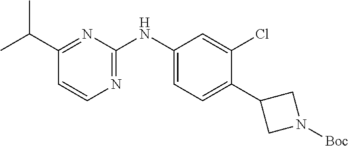 [SMILES: CC(C)c1ccnc(Nc2ccc(C3CN(C(=O)OC(C)(C)C)C3)c(Cl)c2)n1] | 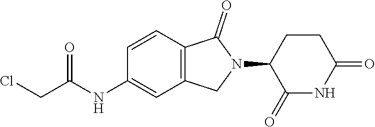 [SMILES: O=C1CC[C@H](N2Cc3cc(NC(=O)CCl)ccc3C2=O)C(=O)N1] |
| 1-145 | 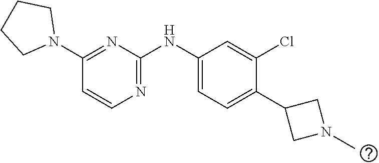 [SMILES: CN1CC(c2ccc(Nc3nccc(N4CCCC4)n3)cc2Cl)C1] | 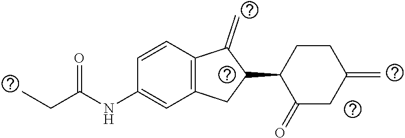 [SMILES: C=C1CC[C@H](C2Cc3cc(NC(=O)CC)ccc3C2=C)C(=O)C1] |
| 1-146 | 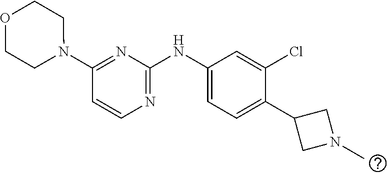 [SMILES: CN1CC(c2ccc(Nc3nccc(N4CCOCC4)n3)cc2Cl)C1] | 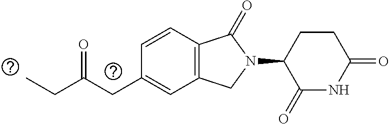 [SMILES: CCC(=O)Cc1ccc2c(c1)CN([C@H]1CCC(=O)NC1=O)C2=O] |
| 1-147 | 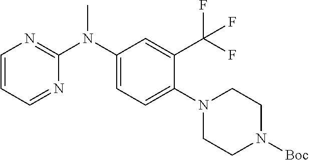 [SMILES: CN(c1ccc(N2CCN(C(=O)OC(C)(C)C)CC2)c(C(F)(F)F)c1)c1ncccn1] | 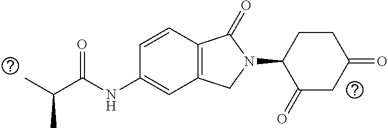 |
| 1-148 | 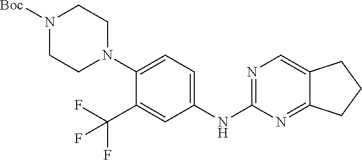 [SMILES: CC(C)(C)OC(=O)N1CCN(c2ccc(Nc3ncc4c(n3)CCC4)cc2C(F)(F)F)CC1] | 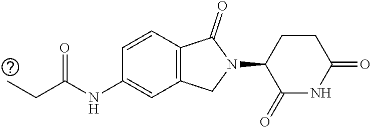 [SMILES: CCC(=O)Nc1ccc2c(c1)CN([C@H]1CCC(=O)NC1=O)C2=O] |
| 1-149 | 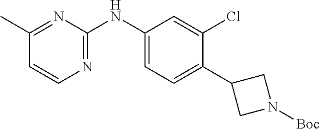 [SMILES: Cc1ccnc(Nc2ccc(C3CN(C(=O)OC(C)(C)C)C3)c(Cl)c2)n1] | 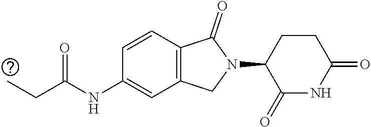 [SMILES: CCC(=O)Nc1ccc2c(c1)CN([C@H]1CCC(=O)NC1=O)C2=O] |
| 1-150 | 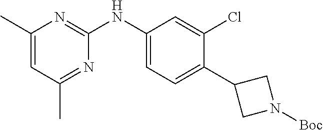 [SMILES: Cc1cc(C)nc(Nc2ccc(C3CN(C(=O)OC(C)(C)C)C3)c(Cl)c2)n1] | 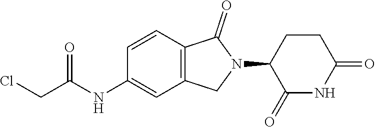 [SMILES: O=C1CC[C@H](N2Cc3cc(NC(=O)CCl)ccc3C2=O)C(=O)N1] |
| 1-151 | 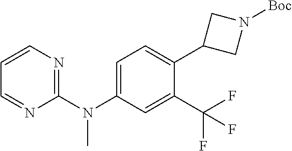 [SMILES: CN(c1ccc(C2CN(C(=O)OC(C)(C)C)C2)c(C(F)(F)F)c1)c1ncccn1] | 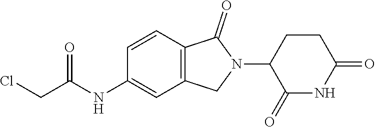 [SMILES: O=C1CCC(N2Cc3cc(NC(=O)CCl)ccc3C2=O)C(=O)N1] |
| 1-152 | 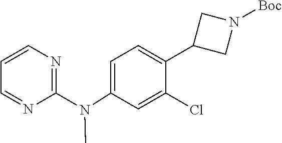 [SMILES: CN(c1ccc(C2CN(C(=O)OC(C)(C)C)C2)c(Cl)c1)c1ncccn1] | 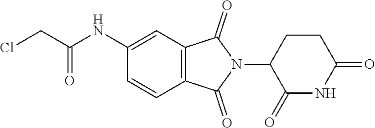 [SMILES: O=C1CCC(N2C(=O)c3ccc(NC(=O)CCl)cc3C2=O)C(=O)N1] |
| 1-153 | 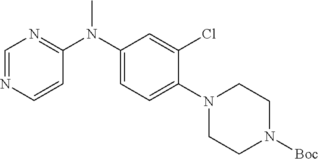 [SMILES: CN(c1ccc(N2CCN(C(=O)OC(C)(C)C)CC2)c(Cl)c1)c1ccncn1] | 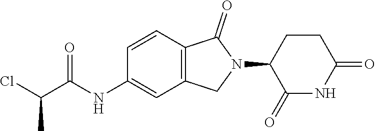 [SMILES: C[C@@H](Cl)C(=O)Nc1ccc2c(c1)CN([C@H]1CCC(=O)NC1=O)C2=O] |
| 1-154 | 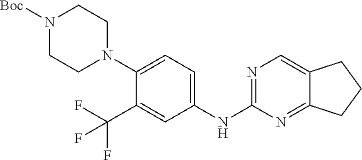 [SMILES: CC(C)(C)OC(=O)N1CCN(c2ccc(Nc3ncc4c(n3)CCC4)cc2C(F)(F)F)CC1] | 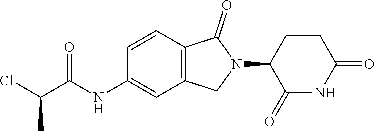 [SMILES: C[C@@H](Cl)C(=O)Nc1ccc2c(c1)CN([C@H]1CCC(=O)NC1=O)C2=O] |
| 1-155 | 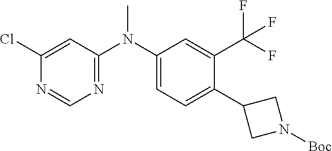 [SMILES: CN(c1ccc(C2CN(C(=O)OC(C)(C)C)C2)c(C(F)(F)F)c1)c1cc(Cl)ncn1] | 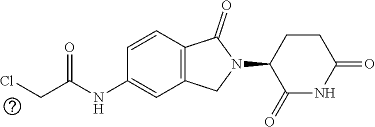 [SMILES: O=C1CC[C@H](N2Cc3cc(NC(=O)CCl)ccc3C2=O)C(=O)N1] |
| 1-156 | 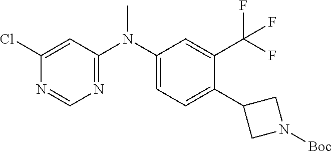 [SMILES: CN(c1ccc(C2CN(C(=O)OC(C)(C)C)C2)c(C(F)(F)F)c1)c1cc(Cl)ncn1] | 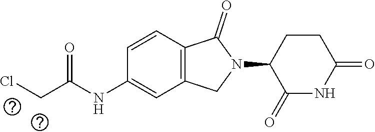 [SMILES: O=C1CC[C@H](N2Cc3cc(NC(=O)CCl)ccc3C2=O)C(=O)N1] |
| 1-157 | 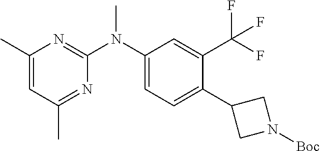 [SMILES: Cc1cc(C)nc(N(C)c2ccc(C3CN(C(=O)OC(C)(C)C)C3)c(C(F)(F)F)c2)n1] | 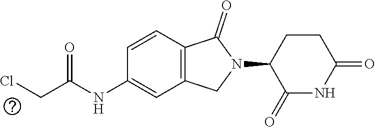 [SMILES: O=C1CC[C@H](N2Cc3cc(NC(=O)CCl)ccc3C2=O)C(=O)N1] |
| 1-158 | 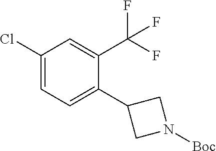 [SMILES: CC(C)(C)OC(=O)N1CC(c2ccc(Cl)cc2C(F)(F)F)C1] | 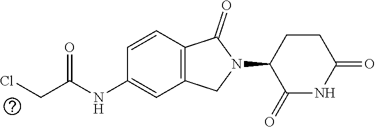 [SMILES: O=C1CC[C@H](N2Cc3cc(NC(=O)CCl)ccc3C2=O)C(=O)N1] |
| 1-159 | 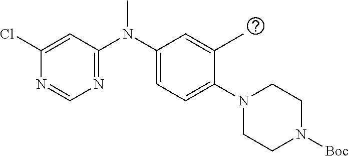 [SMILES: Cc1cc(N(C)c2cc(Cl)ncn2)ccc1N1CCN(C(=O)OC(C)(C)C)CC1] | 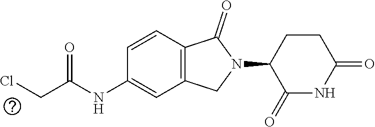 [SMILES: O=C1CC[C@H](N2Cc3cc(NC(=O)CCl)ccc3C2=O)C(=O)N1] |
| 1-160 | 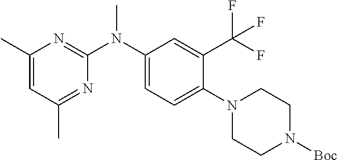 [SMILES: Cc1cc(C)nc(N(C)c2ccc(N3CCN(C(=O)OC(C)(C)C)CC3)c(C(F)(F)F)c2)n1] | 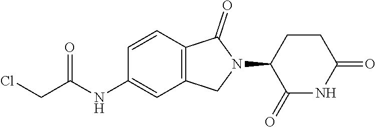 [SMILES: O=C1CC[C@H](N2Cc3cc(NC(=O)CCl)ccc3C2=O)C(=O)N1] |
| 1-161 | 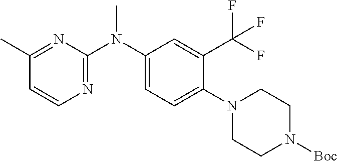 [SMILES: Cc1ccnc(N(C)c2ccc(N3CCN(C(=O)OC(C)(C)C)CC3)c(C(F)(F)F)c2)n1] | 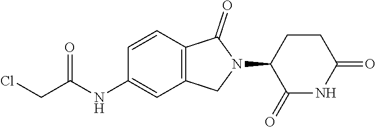 [SMILES: O=C1CC[C@H](N2Cc3cc(NC(=O)CCl)ccc3C2=O)C(=O)N1] |
| 1-162 | 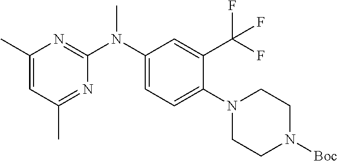 [SMILES: Cc1cc(C)nc(N(C)c2ccc(N3CCN(C(=O)OC(C)(C)C)CC3)c(C(F)(F)F)c2)n1] | 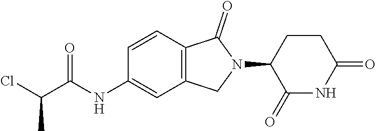 [SMILES: C[C@@H](Cl)C(=O)Nc1ccc2c(c1)CN([C@H]1CCC(=O)NC1=O)C2=O] |
| 1-163 | 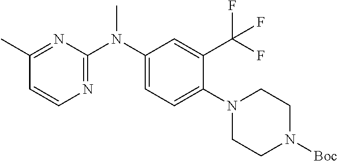 [SMILES: Cc1ccnc(N(C)c2ccc(N3CCN(C(=O)OC(C)(C)C)CC3)c(C(F)(F)F)c2)n1] | 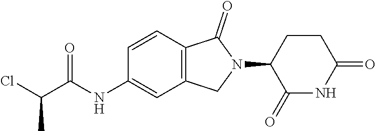 [SMILES: C[C@@H](Cl)C(=O)Nc1ccc2c(c1)CN([C@H]1CCC(=O)NC1=O)C2=O] |
| 1-164 | 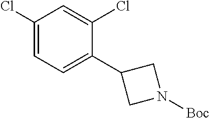 [SMILES: CC(C)(C)OC(=O)N1CC(c2ccc(Cl)cc2Cl)C1] | 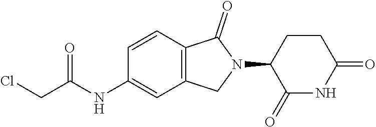 [SMILES: O=C1CC[C@H](N2Cc3cc(NC(=O)CCl)ccc3C2=O)C(=O)N1] |
| 1-165 | 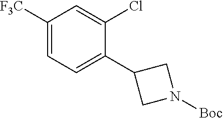 [SMILES: CC(C)(C)OC(=O)N1CC(c2ccc(C(F)(F)F)cc2Cl)C1] | 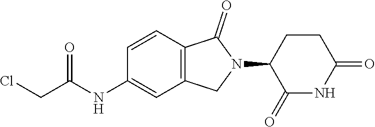 [SMILES: O=C1CC[C@H](N2Cc3cc(NC(=O)CCl)ccc3C2=O)C(=O)N1] |
| 1-166 | 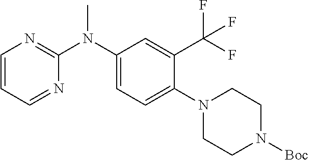 [SMILES: CN(c1ccc(N2CCN(C(=O)OC(C)(C)C)CC2)c(C(F)(F)F)c1)c1ncccn1] | 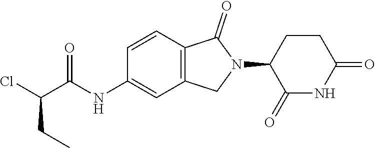 [SMILES: CC[C@@H](Cl)C(=O)Nc1ccc2c(c1)CN([C@H]1CCC(=O)NC1=O)C2=O] |
| 1-167 | 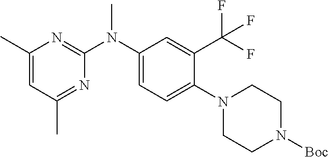 [SMILES: Cc1cc(C)nc(N(C)c2ccc(N3CCN(C(=O)OC(C)(C)C)CC3)c(C(F)(F)F)c2)n1] | 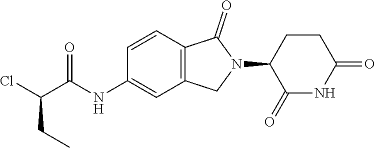 [SMILES: CC[C@@H](Cl)C(=O)Nc1ccc2c(c1)CN([C@H]1CCC(=O)NC1=O)C2=O] |
| 1-168 | 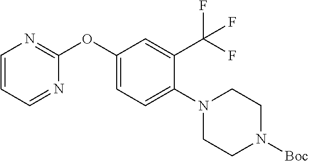 [SMILES: CC(C)(C)OC(=O)N1CCN(c2ccc(Oc3ncccn3)cc2C(F)(F)F)CC1] | 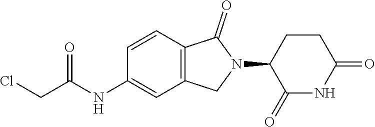 [SMILES: O=C1CC[C@H](N2Cc3cc(NC(=O)CCl)ccc3C2=O)C(=O)N1] |
| 1-169 | 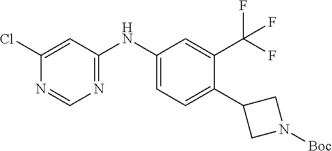 [SMILES: CC(C)(C)OC(=O)N1CC(c2ccc(Nc3cc(Cl)ncn3)cc2C(F)(F)F)C1] | 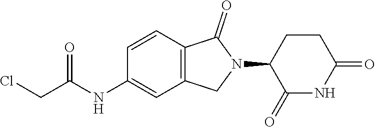 [SMILES: O=C1CC[C@H](N2Cc3cc(NC(=O)CCl)ccc3C2=O)C(=O)N1] |
| 1-170 | 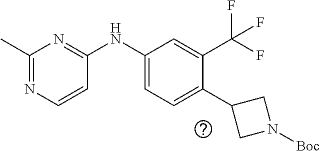 [SMILES: Cc1nccc(Nc2ccc(C3CN(C(=O)OC(C)(C)C)C3)c(C(F)(F)F)c2)n1] | 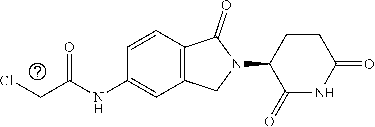 [SMILES: O=C1CC[C@H](N2Cc3cc(NC(=O)CCl)ccc3C2=O)C(=O)N1] |
| 1-171 | 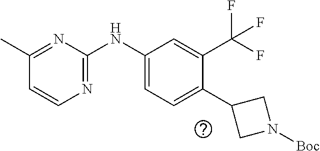 [SMILES: Cc1ccnc(Nc2ccc(C3CN(C(=O)OC(C)(C)C)C3)c(C(F)(F)F)c2)n1] | 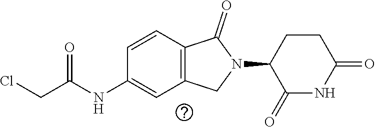 [SMILES: O=C1CC[C@H](N2Cc3cc(NC(=O)CCl)ccc3C2=O)C(=O)N1] |
| 1-172 | 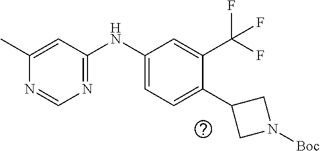 [SMILES: Cc1cc(Nc2ccc(C3CN(C(=O)OC(C)(C)C)C3)c(C(F)(F)F)c2)ncn1] | 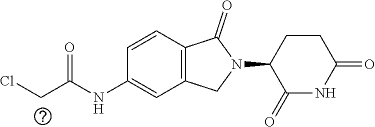 [SMILES: O=C1CC[C@H](N2Cc3cc(NC(=O)CCl)ccc3C2=O)C(=O)N1] |
| 1-173 | 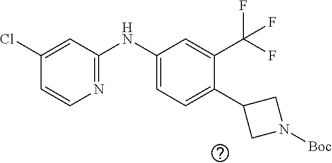 [SMILES: CC(C)(C)OC(=O)N1CC(c2ccc(Nc3cc(Cl)ccn3)cc2C(F)(F)F)C1] | 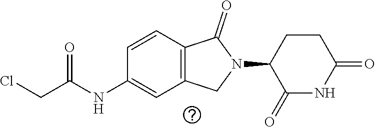 [SMILES: O=C1CC[C@H](N2Cc3cc(NC(=O)CCl)ccc3C2=O)C(=O)N1] |
| 1-174 | 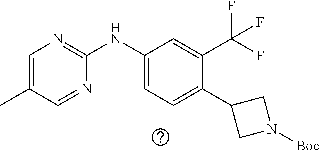 [SMILES: Cc1cnc(Nc2ccc(C3CN(C(=O)OC(C)(C)C)C3)c(C(F)(F)F)c2)nc1] | 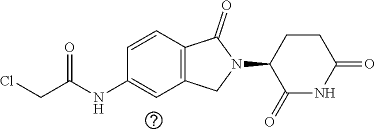 [SMILES: O=C1CC[C@H](N2Cc3cc(NC(=O)CCl)ccc3C2=O)C(=O)N1] |
| 1-175 | 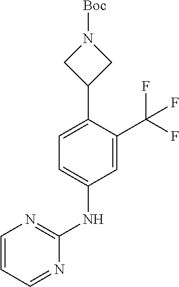 [SMILES: CC(C)(C)OC(=O)N1CC(c2ccc(Nc3ncccn3)cc2C(F)(F)F)C1] | 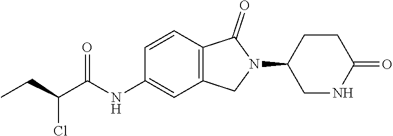 [SMILES: CC[C@H](Cl)C(=O)Nc1ccc2c(c1)CN([C@H]1CCC(=O)NC1)C2=O] |
| 1-176 | 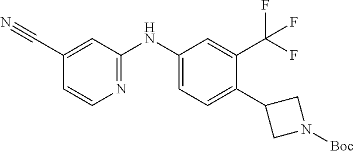 [SMILES: CC(C)(C)OC(=O)N1CC(c2ccc(Nc3cc(C#N)ccn3)cc2C(F)(F)F)C1] | 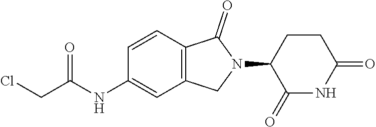 [SMILES: O=C1CC[C@H](N2Cc3cc(NC(=O)CCl)ccc3C2=O)C(=O)N1] |
| 1-177 | 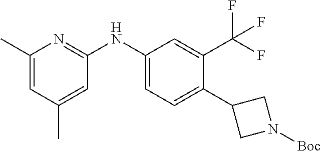 [SMILES: Cc1cc(C)nc(Nc2ccc(C3CN(C(=O)OC(C)(C)C)C3)c(C(F)(F)F)c2)c1] | 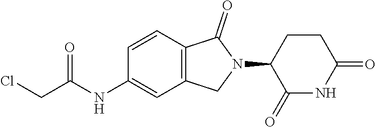 [SMILES: O=C1CC[C@H](N2Cc3cc(NC(=O)CCl)ccc3C2=O)C(=O)N1] |
| 1-178 | 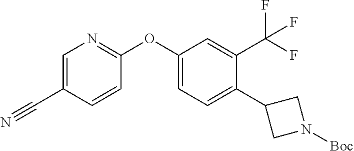 [SMILES: CC(C)(C)OC(=O)N1CC(c2ccc(Oc3ccc(C#N)cn3)cc2C(F)(F)F)C1] | 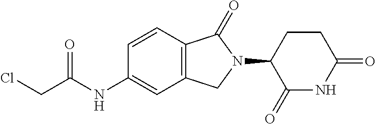 [SMILES: O=C1CC[C@H](N2Cc3cc(NC(=O)CCl)ccc3C2=O)C(=O)N1] |
| 1-179 | 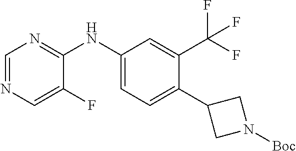 [SMILES: CC(C)(C)OC(=O)N1CC(c2ccc(Nc3ncncc3F)cc2C(F)(F)F)C1] | 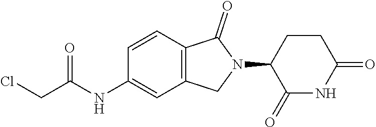 [SMILES: O=C1CC[C@H](N2Cc3cc(NC(=O)CCl)ccc3C2=O)C(=O)N1] |
| 1-180 | 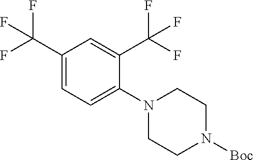 [SMILES: CC(C)(C)OC(=O)N1CCN(c2ccc(C(F)(F)F)cc2C(F)(F)F)CC1] | 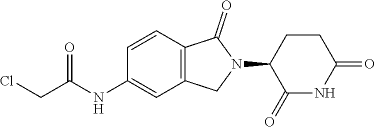 [SMILES: O=C1CC[C@H](N2Cc3cc(NC(=O)CCl)ccc3C2=O)C(=O)N1] |
| 1-181 | 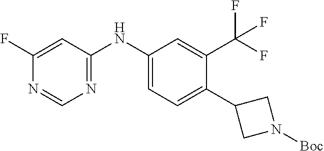 [SMILES: CC(C)(C)OC(=O)N1CC(c2ccc(Nc3cc(F)ncn3)cc2C(F)(F)F)C1] | 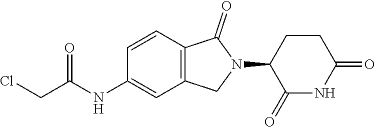 [SMILES: O=C1CC[C@H](N2Cc3cc(NC(=O)CCl)ccc3C2=O)C(=O)N1] |
| 1-182 | 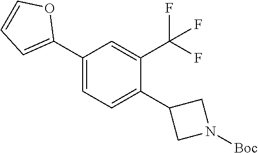 [SMILES: CC(C)(C)OC(=O)N1CC(c2ccc(-c3ccco3)cc2C(F)(F)F)C1] | 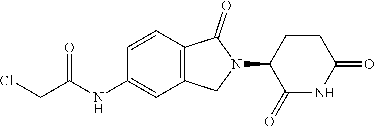 [SMILES: O=C1CC[C@H](N2Cc3cc(NC(=O)CCl)ccc3C2=O)C(=O)N1] |
| 1-183 | 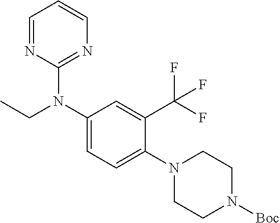 [SMILES: CCN(c1ccc(N2CCN(C(=O)OC(C)(C)C)CC2)c(C(F)(F)F)c1)c1ncccn1] | 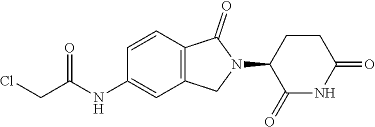 [SMILES: O=C1CC[C@H](N2Cc3cc(NC(=O)CCl)ccc3C2=O)C(=O)N1] |
| 1-184 | 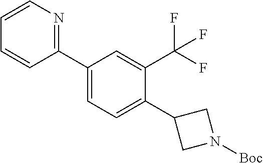 [SMILES: CC(C)(C)OC(=O)N1CC(c2ccc(-c3ccccn3)cc2C(F)(F)F)C1] | 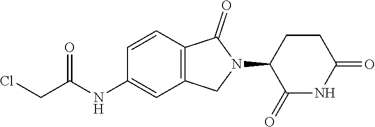 [SMILES: O=C1CC[C@H](N2Cc3cc(NC(=O)CCl)ccc3C2=O)C(=O)N1] |
| 1-185 | 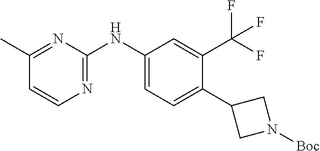 [SMILES: Cc1ccnc(Nc2ccc(C3CN(C(=O)OC(C)(C)C)C3)c(C(F)(F)F)c2)n1] | 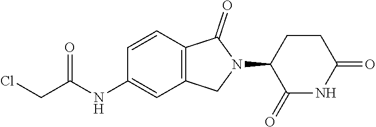 [SMILES: O=C1CC[C@H](N2Cc3cc(NC(=O)CCl)ccc3C2=O)C(=O)N1] |
| 1-186 | 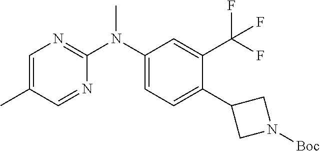 [SMILES: Cc1cnc(N(C)c2ccc(C3CN(C(=O)OC(C)(C)C)C3)c(C(F)(F)F)c2)nc1] | 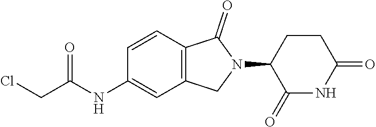 [SMILES: O=C1CC[C@H](N2Cc3cc(NC(=O)CCl)ccc3C2=O)C(=O)N1] |
| 1-187 | 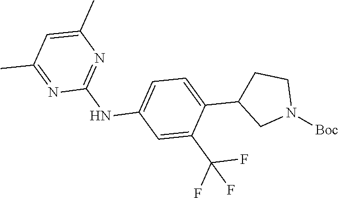 [SMILES: Cc1cc(C)nc(Nc2ccc(C3CCN(C(=O)OC(C)(C)C)C3)c(C(F)(F)F)c2)n1] | 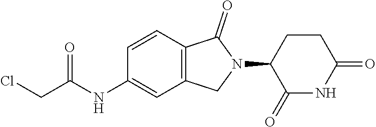 [SMILES: O=C1CC[C@H](N2Cc3cc(NC(=O)CCl)ccc3C2=O)C(=O)N1] |
| 1-188 | 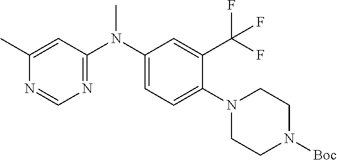 [SMILES: Cc1cc(N(C)c2ccc(N3CCN(C(=O)OC(C)(C)C)CC3)c(C(F)(F)F)c2)ncn1] | 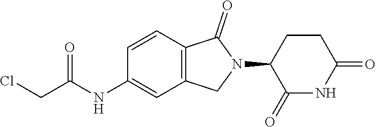 [SMILES: O=C1CC[C@H](N2Cc3cc(NC(=O)CCl)ccc3C2=O)C(=O)N1] |
| 1-189 | 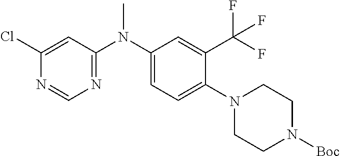 [SMILES: CN(c1ccc(N2CCN(C(=O)OC(C)(C)C)CC2)c(C(F)(F)F)c1)c1cc(Cl)ncn1] | 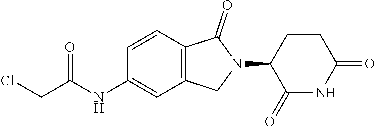 [SMILES: O=C1CC[C@H](N2Cc3cc(NC(=O)CCl)ccc3C2=O)C(=O)N1] |
| 1-190 | 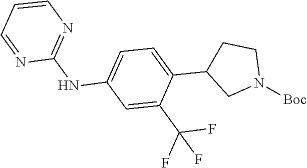 [SMILES: CC(C)(C)OC(=O)N1CCC(c2ccc(Nc3ncccn3)cc2C(F)(F)F)C1] | 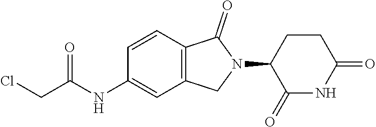 [SMILES: O=C1CC[C@H](N2Cc3cc(NC(=O)CCl)ccc3C2=O)C(=O)N1] |
| 1-191 | 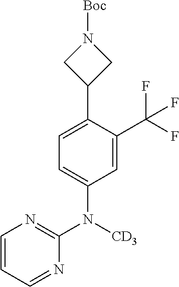 [SMILES: [2H]C([2H])([2H])N(c1ccc(C2CN(C(=O)OC(C)(C)C)C2)c(C(F)(F)F)c1)c1ncccn1] | 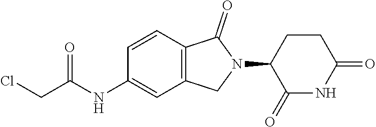 [SMILES: O=C1CC[C@H](N2Cc3cc(NC(=O)CCl)ccc3C2=O)C(=O)N1] |
| 1-192 | 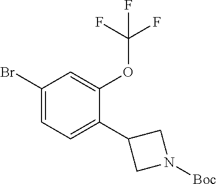 [SMILES: CC(C)(C)OC(=O)N1CC(c2ccc(Br)cc2OC(F)(F)F)C1] | 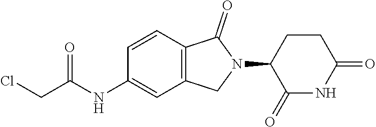 [SMILES: O=C1CC[C@H](N2Cc3cc(NC(=O)CCl)ccc3C2=O)C(=O)N1] |
| 1-193 | 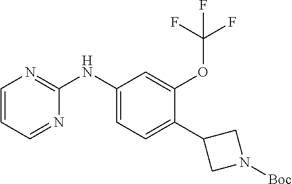 [SMILES: CC(C)(C)OC(=O)N1CC(c2ccc(Nc3ncccn3)cc2OC(F)(F)F)C1] | 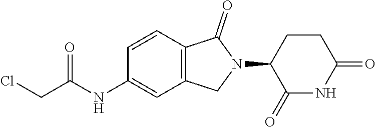 [SMILES: O=C1CC[C@H](N2Cc3cc(NC(=O)CCl)ccc3C2=O)C(=O)N1] |
| 1-194 | 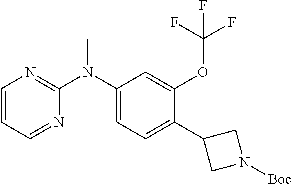 [SMILES: CN(c1ccc(C2CN(C(=O)OC(C)(C)C)C2)c(OC(F)(F)F)c1)c1ncccn1] | 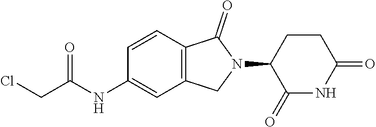 [SMILES: O=C1CC[C@H](N2Cc3cc(NC(=O)CCl)ccc3C2=O)C(=O)N1] |
| 1-195 | 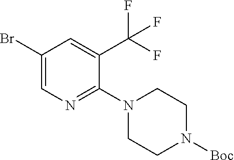 [SMILES: CC(C)(C)OC(=O)N1CCN(c2ncc(Br)cc2C(F)(F)F)CC1] | 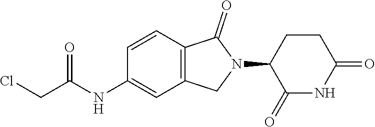 [SMILES: O=C1CC[C@H](N2Cc3cc(NC(=O)CCl)ccc3C2=O)C(=O)N1] |
| 1-196 | 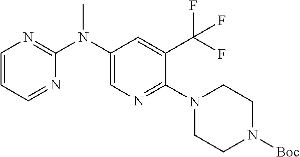 [SMILES: CN(c1cnc(N2CCN(C(=O)OC(C)(C)C)CC2)c(C(F)(F)F)c1)c1ncccn1] | 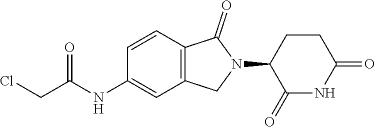 [SMILES: O=C1CC[C@H](N2Cc3cc(NC(=O)CCl)ccc3C2=O)C(=O)N1] |
| 1-197 | 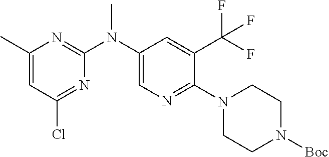 [SMILES: Cc1cc(Cl)nc(N(C)c2cnc(N3CCN(C(=O)OC(C)(C)C)CC3)c(C(F)(F)F)c2)n1] | 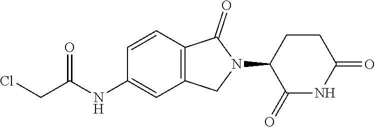 [SMILES: O=C1CC[C@H](N2Cc3cc(NC(=O)CCl)ccc3C2=O)C(=O)N1] |
| 1-198 | 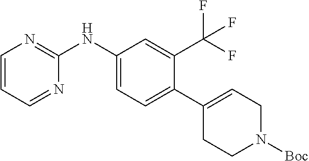 [SMILES: CC(C)(C)OC(=O)N1CC=C(c2ccc(Nc3ncccn3)cc2C(F)(F)F)CC1] | 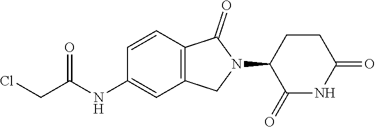 [SMILES: O=C1CC[C@H](N2Cc3cc(NC(=O)CCl)ccc3C2=O)C(=O)N1] |
| 1-199 | 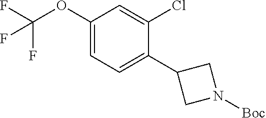 [SMILES: CC(C)(C)OC(=O)N1CC(c2ccc(OC(F)(F)F)cc2Cl)C1] | 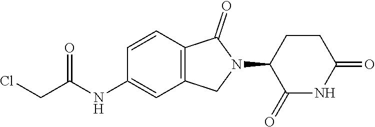 [SMILES: O=C1CC[C@H](N2Cc3cc(NC(=O)CCl)ccc3C2=O)C(=O)N1] |
| 1-200 | 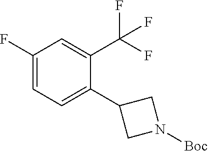 [SMILES: CC(C)(C)OC(=O)N1CC(c2ccc(F)cc2C(F)(F)F)C1] | 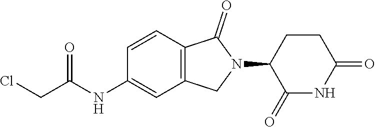 [SMILES: O=C1CC[C@H](N2Cc3cc(NC(=O)CCl)ccc3C2=O)C(=O)N1] |
| 1-201 | 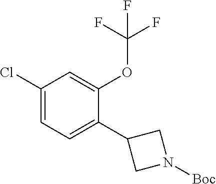 [SMILES: CC(C)(C)OC(=O)N1CC(c2ccc(Cl)cc2OC(F)(F)F)C1] | 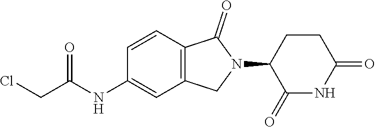 [SMILES: O=C1CC[C@H](N2Cc3cc(NC(=O)CCl)ccc3C2=O)C(=O)N1] |
| 1-202 | 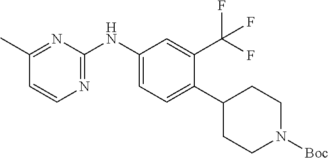 [SMILES: Cc1ccnc(Nc2ccc(C3CCN(C(=O)OC(C)(C)C)CC3)c(C(F)(F)F)c2)n1] | 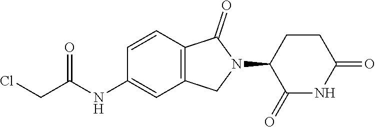 [SMILES: O=C1CC[C@H](N2Cc3cc(NC(=O)CCl)ccc3C2=O)C(=O)N1] |
| 1-203 | 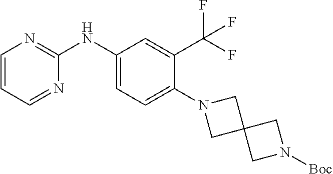 [SMILES: CC(C)(C)OC(=O)N1CC2(C1)CN(c1ccc(Nc3ncccn3)cc1C(F)(F)F)C2] | 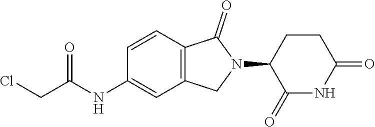 [SMILES: O=C1CC[C@H](N2Cc3cc(NC(=O)CCl)ccc3C2=O)C(=O)N1] |
| 1-204 | 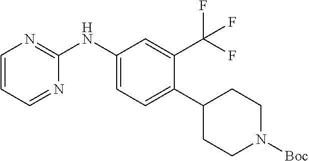 [SMILES: CC(C)(C)OC(=O)N1CCC(c2ccc(Nc3ncccn3)cc2C(F)(F)F)CC1] | 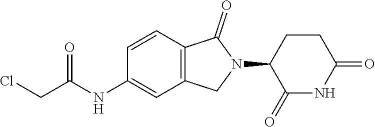 [SMILES: O=C1CC[C@H](N2Cc3cc(NC(=O)CCl)ccc3C2=O)C(=O)N1] |
| 1-205 | 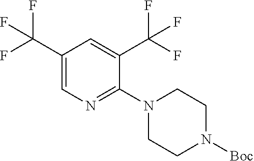 [SMILES: CC(C)(C)OC(=O)N1CCN(c2ncc(C(F)(F)F)cc2C(F)(F)F)CC1] | 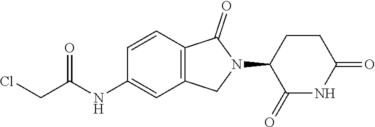 [SMILES: O=C1CC[C@H](N2Cc3cc(NC(=O)CCl)ccc3C2=O)C(=O)N1] |
| 1-206 | 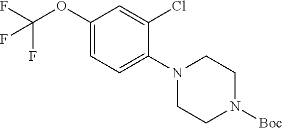 [SMILES: CC(C)(C)OC(=O)N1CCN(c2ccc(OC(F)(F)F)cc2Cl)CC1] | 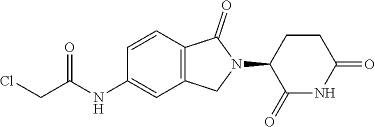 [SMILES: O=C1CC[C@H](N2Cc3cc(NC(=O)CCl)ccc3C2=O)C(=O)N1] |
| 1-207 | 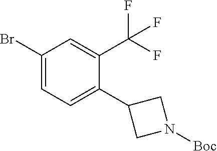 [SMILES: CC(C)(C)OC(=O)N1CC(c2ccc(Br)cc2C(F)(F)F)C1] | 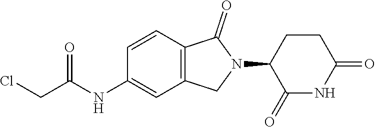 [SMILES: O=C1CC[C@H](N2Cc3cc(NC(=O)CCl)ccc3C2=O)C(=O)N1] |
| 1-208 | 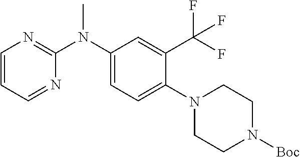 [SMILES: CN(c1ccc(N2CCN(C(=O)OC(C)(C)C)CC2)c(C(F)(F)F)c1)c1ncccn1] | 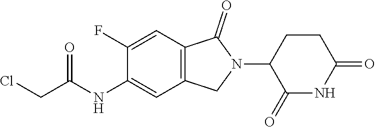 [SMILES: O=C1CCC(N2Cc3cc(NC(=O)CCl)c(F)cc3C2=O)C(=O)N1] |
| 1-209 | 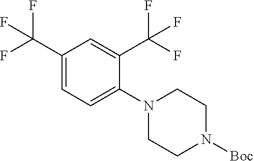 [SMILES: CC(C)(C)OC(=O)N1CCN(c2ccc(C(F)(F)F)cc2C(F)(F)F)CC1] | 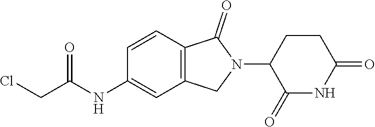 [SMILES: O=C1CCC(N2Cc3cc(NC(=O)CCl)ccc3C2=O)C(=O)N1] |
| 1-210 | 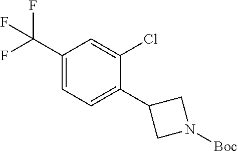 [SMILES: CC(C)(C)OC(=O)N1CC(c2ccc(C(F)(F)F)cc2Cl)C1] | 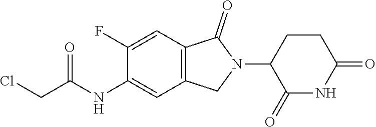 [SMILES: O=C1CCC(N2Cc3cc(NC(=O)CCl)c(F)cc3C2=O)C(=O)N1] |
| 1-211 | 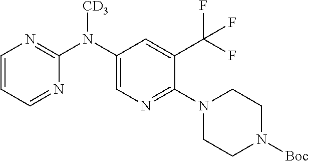 [SMILES: [2H]C([2H])([2H])N(c1cnc(N2CCN(C(=O)OC(C)(C)C)CC2)c(C(F)(F)F)c1)c1ncccn1] | 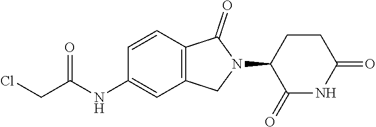 [SMILES: O=C1CC[C@H](N2Cc3cc(NC(=O)CCl)ccc3C2=O)C(=O)N1] |
| 1-212 | 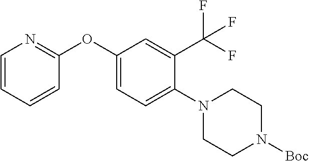 [SMILES: CC(C)(C)OC(=O)N1CCN(c2ccc(Oc3ccccn3)cc2C(F)(F)F)CC1] | 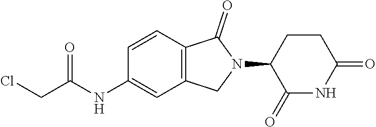 [SMILES: O=C1CC[C@H](N2Cc3cc(NC(=O)CCl)ccc3C2=O)C(=O)N1] |
| 1-213 | 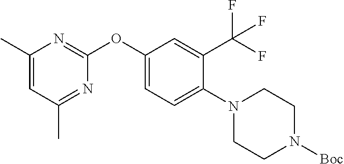 [SMILES: Cc1cc(C)nc(Oc2ccc(N3CCN(C(=O)OC(C)(C)C)CC3)c(C(F)(F)F)c2)n1] | 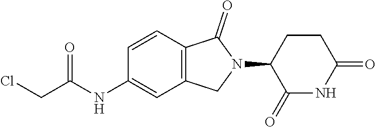 [SMILES: O=C1CC[C@H](N2Cc3cc(NC(=O)CCl)ccc3C2=O)C(=O)N1] |
| 1-214 | 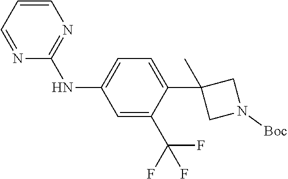 [SMILES: CC(C)(C)OC(=O)N1CC(C)(c2ccc(Nc3ncccn3)cc2C(F)(F)F)C1] | 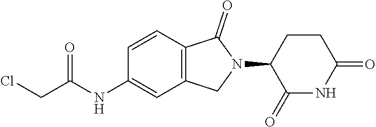 [SMILES: O=C1CC[C@H](N2Cc3cc(NC(=O)CCl)ccc3C2=O)C(=O)N1] |
| 1-215 | 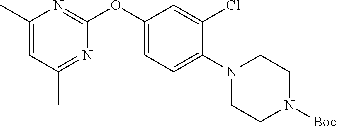 [SMILES: Cc1cc(C)nc(Oc2ccc(N3CCN(C(=O)OC(C)(C)C)CC3)c(Cl)c2)n1] | 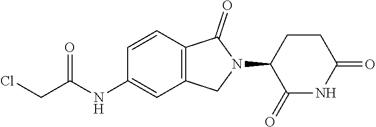 [SMILES: O=C1CC[C@H](N2Cc3cc(NC(=O)CCl)ccc3C2=O)C(=O)N1] |
| 1-216 | 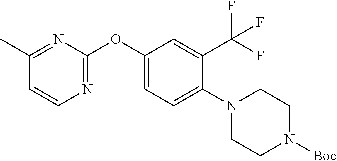 [SMILES: Cc1ccnc(Oc2ccc(N3CCN(C(=O)OC(C)(C)C)CC3)c(C(F)(F)F)c2)n1] | 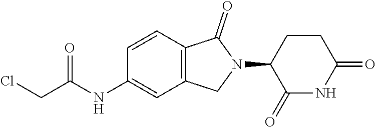 [SMILES: O=C1CC[C@H](N2Cc3cc(NC(=O)CCl)ccc3C2=O)C(=O)N1] |
| 1-217 | 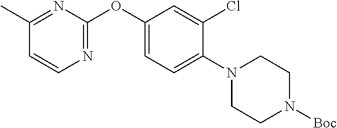 [SMILES: Cc1ccnc(Oc2ccc(N3CCN(C(=O)OC(C)(C)C)CC3)c(Cl)c2)n1] | 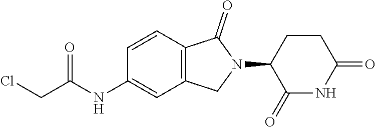 [SMILES: O=C1CC[C@H](N2Cc3cc(NC(=O)CCl)ccc3C2=O)C(=O)N1] |
| 1-218 | 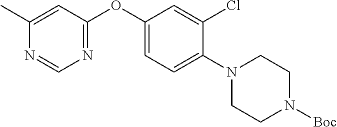 [SMILES: Cc1cc(Oc2ccc(N3CCN(C(=O)OC(C)(C)C)CC3)c(Cl)c2)ncn1] | 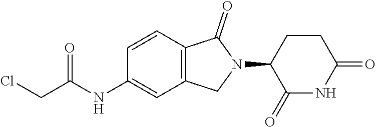 [SMILES: O=C1CC[C@H](N2Cc3cc(NC(=O)CCl)ccc3C2=O)C(=O)N1] |
| 1-219 | 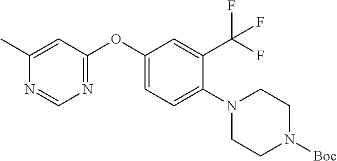 [SMILES: Cc1cc(Oc2ccc(N3CCN(C(=O)OC(C)(C)C)CC3)c(C(F)(F)F)c2)ncn1] | 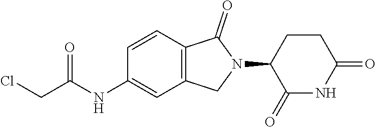 [SMILES: O=C1CC[C@H](N2Cc3cc(NC(=O)CCl)ccc3C2=O)C(=O)N1] |
| 1-220 | 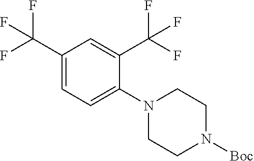 [SMILES: CC(C)(C)OC(=O)N1CCN(c2ccc(C(F)(F)F)cc2C(F)(F)F)CC1] | 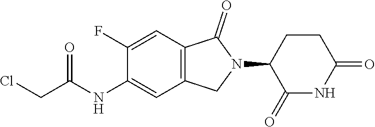 [SMILES: O=C1CC[C@H](N2Cc3cc(NC(=O)CCl)c(F)cc3C2=O)C(=O)N1] |
| 1-221 | 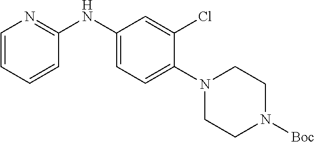 [SMILES: CC(C)(C)OC(=O)N1CCN(c2ccc(Nc3ccccn3)cc2Cl)CC1] | 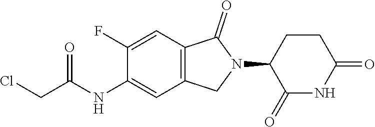 [SMILES: O=C1CC[C@H](N2Cc3cc(NC(=O)CCl)c(F)cc3C2=O)C(=O)N1] |
| 1-222 | 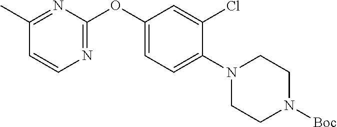 [SMILES: Cc1ccnc(Oc2ccc(N3CCN(C(=O)OC(C)(C)C)CC3)c(Cl)c2)n1] | 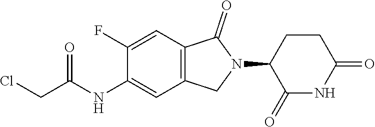 [SMILES: O=C1CC[C@H](N2Cc3cc(NC(=O)CCl)c(F)cc3C2=O)C(=O)N1] |
| 1-223 | 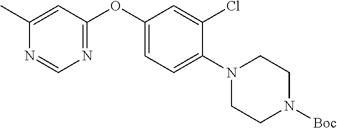 [SMILES: Cc1cc(Oc2ccc(N3CCN(C(=O)OC(C)(C)C)CC3)c(Cl)c2)ncn1] | 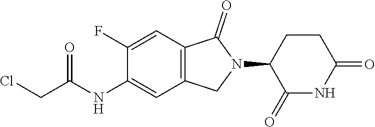 [SMILES: O=C1CC[C@H](N2Cc3cc(NC(=O)CCl)c(F)cc3C2=O)C(=O)N1] |
| 1-224 | 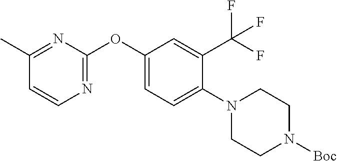 [SMILES: Cc1ccnc(Oc2ccc(N3CCN(C(=O)OC(C)(C)C)CC3)c(C(F)(F)F)c2)n1] | 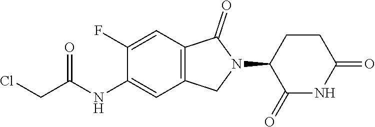 [SMILES: O=C1CC[C@H](N2Cc3cc(NC(=O)CCl)c(F)cc3C2=O)C(=O)N1] |
| 1-225 | 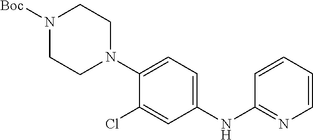 [SMILES: CC(C)(C)OC(=O)N1CCN(c2ccc(Nc3ccccn3)cc2Cl)CC1] | 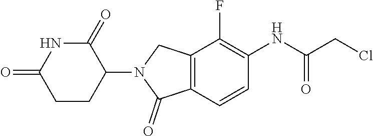 [SMILES: O=C1CCC(N2Cc3c(ccc(NC(=O)CCl)c3F)C2=O)C(=O)N1] |
| 1-226 | 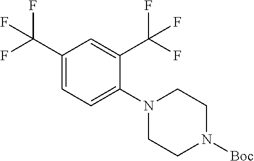 [SMILES: CC(C)(C)OC(=O)N1CCN(c2ccc(C(F)(F)F)cc2C(F)(F)F)CC1] | 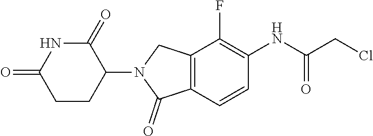 [SMILES: O=C1CCC(N2Cc3c(ccc(NC(=O)CCl)c3F)C2=O)C(=O)N1] |
| 1-227 | 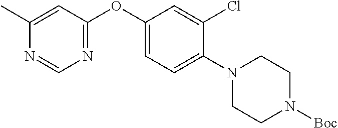 [SMILES: Cc1cc(Oc2ccc(N3CCN(C(=O)OC(C)(C)C)CC3)c(Cl)c2)ncn1] | 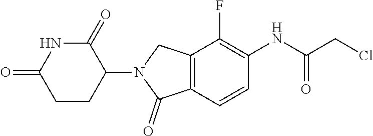 [SMILES: O=C1CCC(N2Cc3c(ccc(NC(=O)CCl)c3F)C2=O)C(=O)N1] |
| 1-228 | 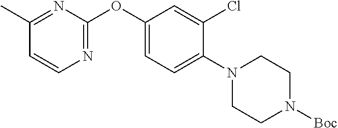 [SMILES: Cc1ccnc(Oc2ccc(N3CCN(C(=O)OC(C)(C)C)CC3)c(Cl)c2)n1] | 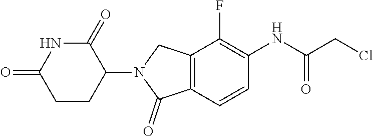 [SMILES: O=C1CCC(N2Cc3c(ccc(NC(=O)CCl)c3F)C2=O)C(=O)N1] |
| 1-229 | 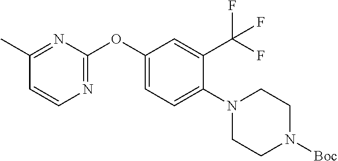 [SMILES: Cc1ccnc(Oc2ccc(N3CCN(C(=O)OC(C)(C)C)CC3)c(C(F)(F)F)c2)n1] | 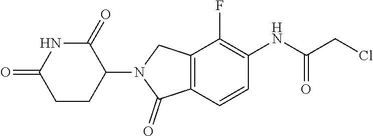 [SMILES: O=C1CCC(N2Cc3c(ccc(NC(=O)CCl)c3F)C2=O)C(=O)N1] |
| 1-230 | 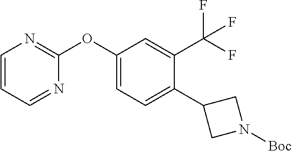 [SMILES: CC(C)(C)OC(=O)N1CC(c2ccc(Oc3ncccn3)cc2C(F)(F)F)C1] | 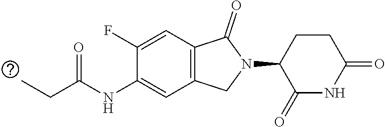 [SMILES: CCC(=O)Nc1cc2c(cc1F)C(=O)N([C@H]1CCC(=O)NC1=O)C2] |
| 1-231 | 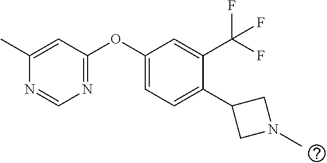 | 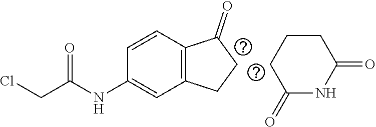 |
| 1-232 | 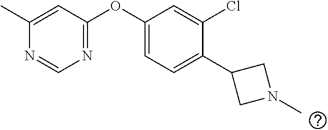 | 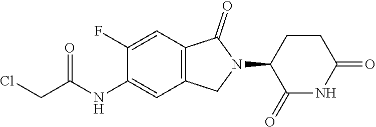 [SMILES: O=C1CC[C@H](N2Cc3cc(NC(=O)CCl)c(F)cc3C2=O)C(=O)N1] |
| 1-233 | 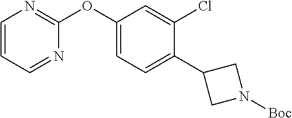 [SMILES: CC(C)(C)OC(=O)N1CC(c2ccc(Oc3ncccn3)cc2Cl)C1] | 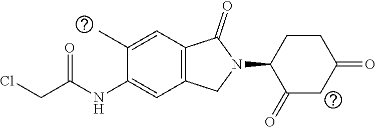 [SMILES: Cc1cc2c(cc1NC(=O)CCl)CN([C@H]1CCC(=O)CC1=O)C2=O] |
| 1-234 | 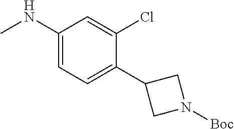 [SMILES: CNc1ccc(C2CN(C(=O)OC(C)(C)C)C2)c(Cl)c1] | 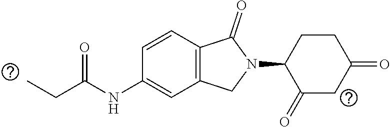 [SMILES: CCC(=O)Nc1ccc2c(c1)CN([C@H]1CCC(=O)CC1=O)C2=O] |
| 1-235 | 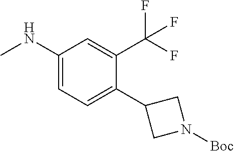 [SMILES: CNc1ccc(C2CN(C(=O)OC(C)(C)C)C2)c(C(F)(F)F)c1] | 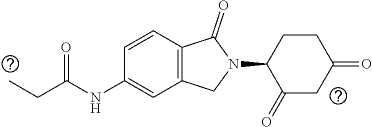 [SMILES: CCC(=O)Nc1ccc2c(c1)CN([C@H]1CCC(=O)CC1=O)C2=O] |
| 1-236 | 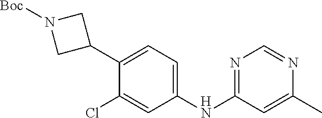 [SMILES: Cc1cc(Nc2ccc(C3CN(C(=O)OC(C)(C)C)C3)c(Cl)c2)ncn1] | 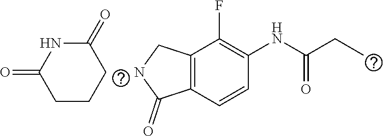 |
| 1-237 | 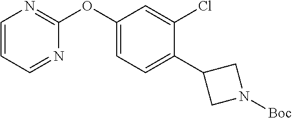 [SMILES: CC(C)(C)OC(=O)N1CC(c2ccc(Oc3ncccn3)cc2Cl)C1] | 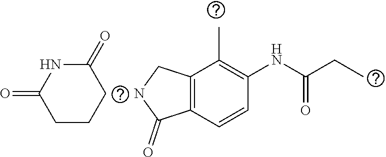 |
| 1-238 | 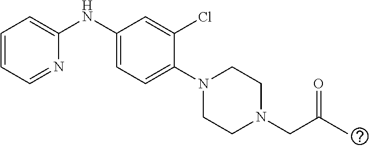 [SMILES: CC(=O)CN1CCN(c2ccc(Nc3ccccn3)cc2Cl)CC1] | 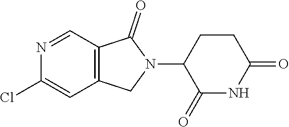 [SMILES: O=C1CCC(N2Cc3cc(Cl)ncc3C2=O)C(=O)N1] |
| 1-239 | 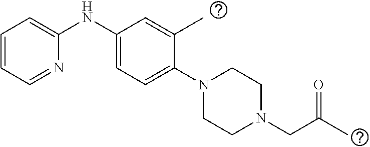 [SMILES: CC(=O)CN1CCN(c2ccc(Nc3ccccn3)cc2C)CC1] | 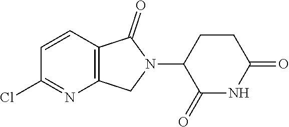 [SMILES: O=C1CCC(N2Cc3nc(Cl)ccc3C2=O)C(=O)N1] |
| 1-240 | 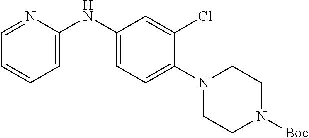 [SMILES: CC(C)(C)OC(=O)N1CCN(c2ccc(Nc3ccccn3)cc2Cl)CC1] | 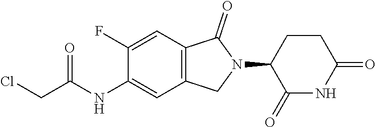 [SMILES: O=C1CC[C@H](N2Cc3cc(NC(=O)CCl)c(F)cc3C2=O)C(=O)N1] |
| 1-241 | 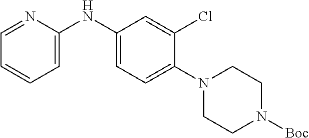 [SMILES: CC(C)(C)OC(=O)N1CCN(c2ccc(Nc3ccccn3)cc2Cl)CC1] | 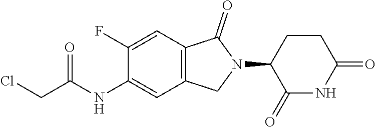 [SMILES: O=C1CC[C@H](N2Cc3cc(NC(=O)CCl)c(F)cc3C2=O)C(=O)N1] |
| 1-242 | 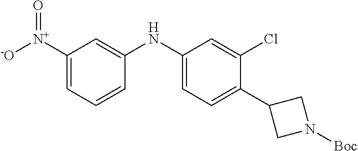 | 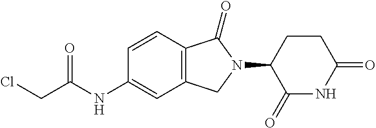 [SMILES: O=C1CC[C@H](N2Cc3cc(NC(=O)CCl)ccc3C2=O)C(=O)N1] |
| 1-243 | 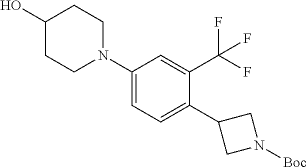 [SMILES: CC(C)(C)OC(=O)N1CC(c2ccc(N3CCC(O)CC3)cc2C(F)(F)F)C1] | 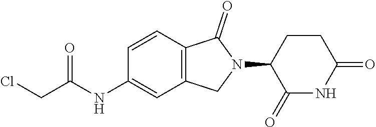 [SMILES: O=C1CC[C@H](N2Cc3cc(NC(=O)CCl)ccc3C2=O)C(=O)N1] |
| 1-244 | 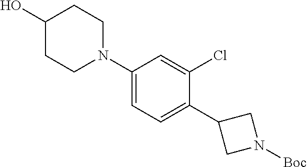 [SMILES: CC(C)(C)OC(=O)N1CC(c2ccc(N3CCC(O)CC3)cc2Cl)C1] | 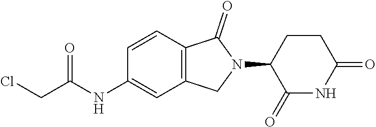 [SMILES: O=C1CC[C@H](N2Cc3cc(NC(=O)CCl)ccc3C2=O)C(=O)N1] |
| 1-245 | 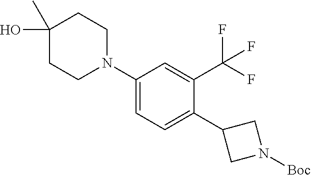 [SMILES: CC1(O)CCN(c2ccc(C3CN(C(=O)OC(C)(C)C)C3)c(C(F)(F)F)c2)CC1] | 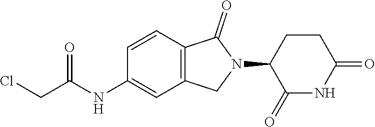 [SMILES: O=C1CC[C@H](N2Cc3cc(NC(=O)CCl)ccc3C2=O)C(=O)N1] |
| 1-246 | 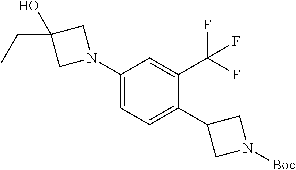 [SMILES: CCC1(O)CN(c2ccc(C3CN(C(=O)OC(C)(C)C)C3)c(C(F)(F)F)c2)C1] | 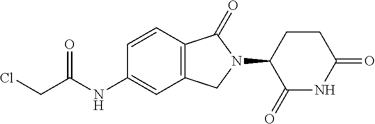 [SMILES: O=C1CC[C@H](N2Cc3cc(NC(=O)CCl)ccc3C2=O)C(=O)N1] |
| 1-247 | 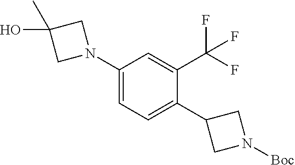 [SMILES: CC1(O)CN(c2ccc(C3CN(C(=O)OC(C)(C)C)C3)c(C(F)(F)F)c2)C1] | 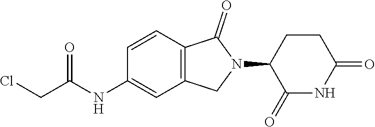 [SMILES: O=C1CC[C@H](N2Cc3cc(NC(=O)CCl)ccc3C2=O)C(=O)N1] |
| 1-248 | 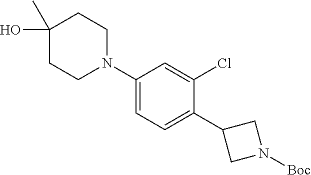 [SMILES: CC1(O)CCN(c2ccc(C3CN(C(=O)OC(C)(C)C)C3)c(Cl)c2)CC1] | 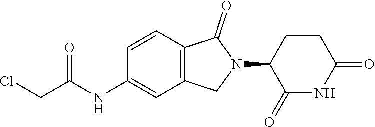 [SMILES: O=C1CC[C@H](N2Cc3cc(NC(=O)CCl)ccc3C2=O)C(=O)N1] |
| 1-249 | 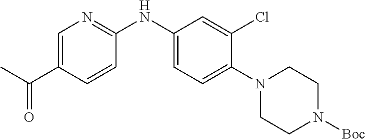 [SMILES: CC(=O)c1ccc(Nc2ccc(N3CCN(C(=O)OC(C)(C)C)CC3)c(Cl)c2)nc1] | 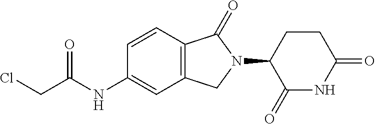 [SMILES: O=C1CC[C@H](N2Cc3cc(NC(=O)CCl)ccc3C2=O)C(=O)N1] |
| 1-250 | 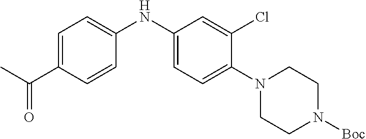 [SMILES: CC(=O)c1ccc(Nc2ccc(N3CCN(C(=O)OC(C)(C)C)CC3)c(Cl)c2)cc1] | 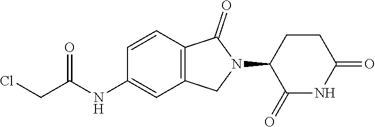 [SMILES: O=C1CC[C@H](N2Cc3cc(NC(=O)CCl)ccc3C2=O)C(=O)N1] |
| 1-251 | 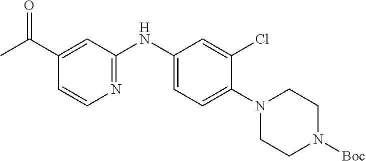 [SMILES: CC(=O)c1ccnc(Nc2ccc(N3CCN(C(=O)OC(C)(C)C)CC3)c(Cl)c2)c1] | 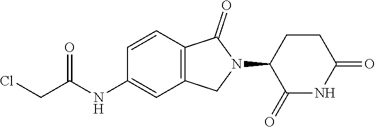 [SMILES: O=C1CC[C@H](N2Cc3cc(NC(=O)CCl)ccc3C2=O)C(=O)N1] |
| 1-252 | 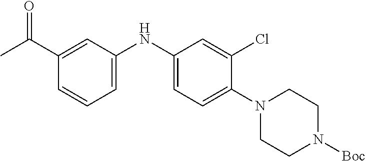 [SMILES: CC(=O)c1cccc(Nc2ccc(N3CCN(C(=O)OC(C)(C)C)CC3)c(Cl)c2)c1] | 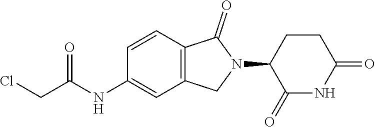 [SMILES: O=C1CC[C@H](N2Cc3cc(NC(=O)CCl)ccc3C2=O)C(=O)N1] |
| 1-253 | 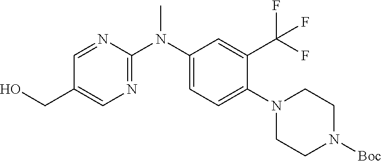 [SMILES: CN(c1ccc(N2CCN(C(=O)OC(C)(C)C)CC2)c(C(F)(F)F)c1)c1ncc(CO)cn1] | 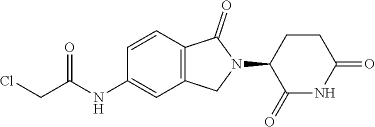 [SMILES: O=C1CC[C@H](N2Cc3cc(NC(=O)CCl)ccc3C2=O)C(=O)N1] |
| 1-254 | 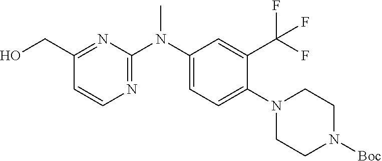 [SMILES: CN(c1ccc(N2CCN(C(=O)OC(C)(C)C)CC2)c(C(F)(F)F)c1)c1nccc(CO)n1] | 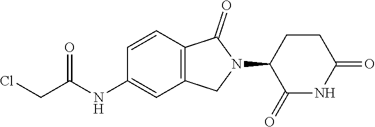 [SMILES: O=C1CC[C@H](N2Cc3cc(NC(=O)CCl)ccc3C2=O)C(=O)N1] |
| 1-255 | 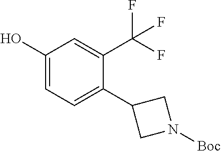 [SMILES: CC(C)(C)OC(=O)N1CC(c2ccc(O)cc2C(F)(F)F)C1] | 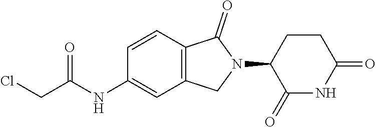 [SMILES: O=C1CC[C@H](N2Cc3cc(NC(=O)CCl)ccc3C2=O)C(=O)N1] |
| 1-256 | 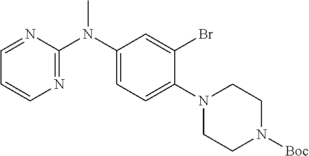 [SMILES: CN(c1ccc(N2CCN(C(=O)OC(C)(C)C)CC2)c(Br)c1)c1ncccn1] | 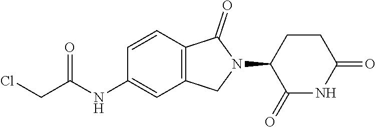 [SMILES: O=C1CC[C@H](N2Cc3cc(NC(=O)CCl)ccc3C2=O)C(=O)N1] |
| 1-257 | 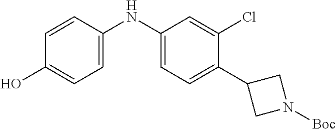 [SMILES: CC(C)(C)OC(=O)N1CC(c2ccc(Nc3ccc(O)cc3)cc2Cl)C1] | 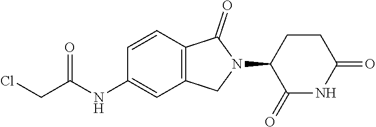 [SMILES: O=C1CC[C@H](N2Cc3cc(NC(=O)CCl)ccc3C2=O)C(=O)N1] |
| 1-258 | 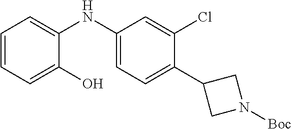 [SMILES: CC(C)(C)OC(=O)N1CC(c2ccc(Nc3ccccc3O)cc2Cl)C1] | 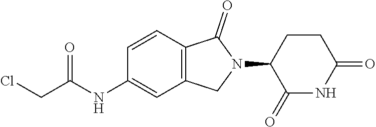 [SMILES: O=C1CC[C@H](N2Cc3cc(NC(=O)CCl)ccc3C2=O)C(=O)N1] |
| 1-259 | 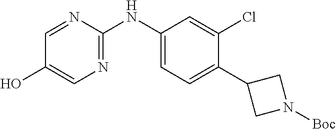 [SMILES: CC(C)(C)OC(=O)N1CC(c2ccc(Nc3ncc(O)cn3)cc2Cl)C1] | 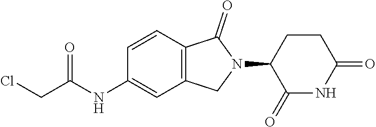 [SMILES: O=C1CC[C@H](N2Cc3cc(NC(=O)CCl)ccc3C2=O)C(=O)N1] |
| 1-260 | 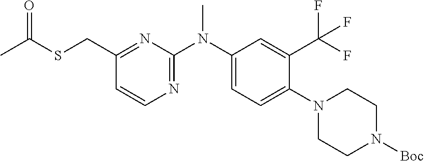 [SMILES: CC(=O)SCc1ccnc(N(C)c2ccc(N3CCN(C(=O)OC(C)(C)C)CC3)c(C(F)(F)F)c2)n1] | 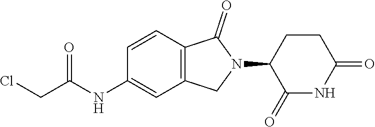 [SMILES: O=C1CC[C@H](N2Cc3cc(NC(=O)CCl)ccc3C2=O)C(=O)N1] |
| 1-261 | 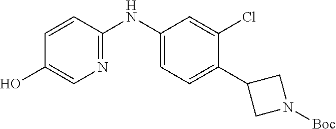 [SMILES: CC(C)(C)OC(=O)N1CC(c2ccc(Nc3ccc(O)cn3)cc2Cl)C1] | 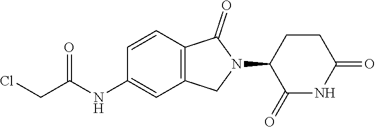 [SMILES: O=C1CC[C@H](N2Cc3cc(NC(=O)CCl)ccc3C2=O)C(=O)N1] |
| 1-262 | 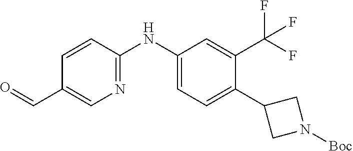 [SMILES: CC(C)(C)OC(=O)N1CC(c2ccc(Nc3ccc(C=O)cn3)cc2C(F)(F)F)C1] | 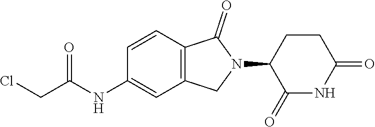 [SMILES: O=C1CC[C@H](N2Cc3cc(NC(=O)CCl)ccc3C2=O)C(=O)N1] |
| 1-263 | 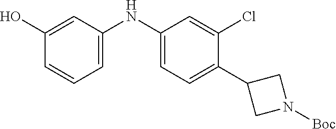 [SMILES: CC(C)(C)OC(=O)N1CC(c2ccc(Nc3cccc(O)c3)cc2Cl)C1] | 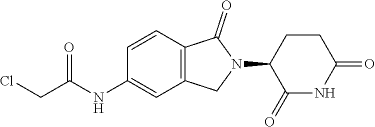 [SMILES: O=C1CC[C@H](N2Cc3cc(NC(=O)CCl)ccc3C2=O)C(=O)N1] |
| 1-264 | 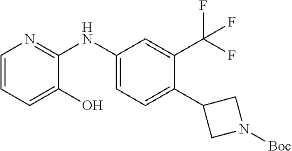 [SMILES: CC(C)(C)OC(=O)N1CC(c2ccc(Nc3ncccc3O)cc2C(F)(F)F)C1] | 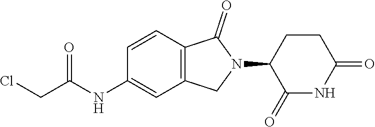 [SMILES: O=C1CC[C@H](N2Cc3cc(NC(=O)CCl)ccc3C2=O)C(=O)N1] |
| 1-265 | 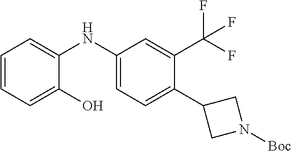 [SMILES: CC(C)(C)OC(=O)N1CC(c2ccc(Nc3ccccc3O)cc2C(F)(F)F)C1] | 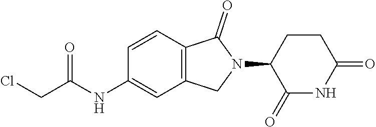 [SMILES: O=C1CC[C@H](N2Cc3cc(NC(=O)CCl)ccc3C2=O)C(=O)N1] |
| 1-266 | 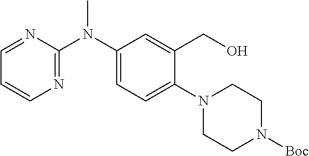 [SMILES: CN(c1ccc(N2CCN(C(=O)OC(C)(C)C)CC2)c(CO)c1)c1ncccn1] | 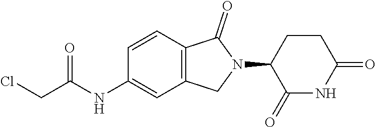 [SMILES: O=C1CC[C@H](N2Cc3cc(NC(=O)CCl)ccc3C2=O)C(=O)N1] |
| 1-267 | 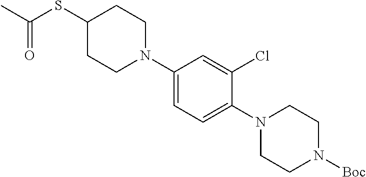 [SMILES: CC(=O)SC1CCN(c2ccc(N3CCN(C(=O)OC(C)(C)C)CC3)c(Cl)c2)CC1] | 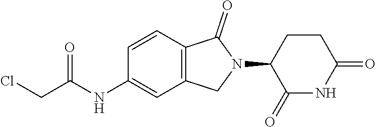 [SMILES: O=C1CC[C@H](N2Cc3cc(NC(=O)CCl)ccc3C2=O)C(=O)N1] |
| 1-268 | 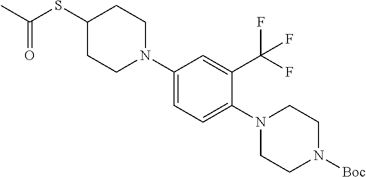 [SMILES: CC(=O)SC1CCN(c2ccc(N3CCN(C(=O)OC(C)(C)C)CC3)c(C(F)(F)F)c2)CC1] | 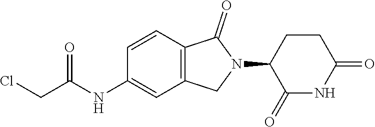 [SMILES: O=C1CC[C@H](N2Cc3cc(NC(=O)CCl)ccc3C2=O)C(=O)N1] |
| 1-269 | 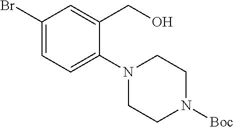 [SMILES: CC(C)(C)OC(=O)N1CCN(c2ccc(Br)cc2CO)CC1] | 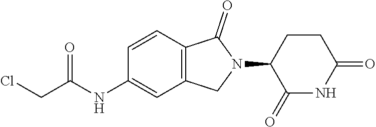 [SMILES: O=C1CC[C@H](N2Cc3cc(NC(=O)CCl)ccc3C2=O)C(=O)N1] |
| 1-270 | 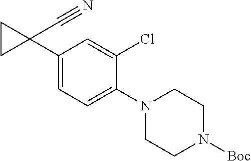 [SMILES: CC(C)(C)OC(=O)N1CCN(c2ccc(C3(C#N)CC3)cc2Cl)CC1] | 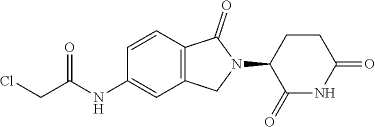 [SMILES: O=C1CC[C@H](N2Cc3cc(NC(=O)CCl)ccc3C2=O)C(=O)N1] |
| 1-271 | 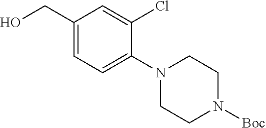 [SMILES: CC(C)(C)OC(=O)N1CCN(c2ccc(CO)cc2Cl)CC1] | 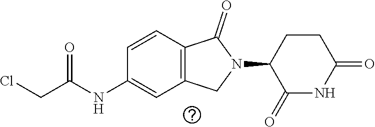 [SMILES: O=C1CC[C@H](N2Cc3cc(NC(=O)CCl)ccc3C2=O)C(=O)N1] |
| 1-272 | 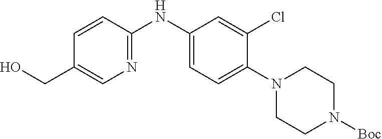 [SMILES: CC(C)(C)OC(=O)N1CCN(c2ccc(Nc3ccc(CO)cn3)cc2Cl)CC1] | 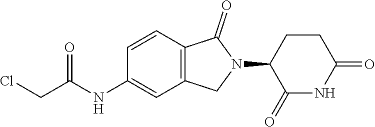 [SMILES: O=C1CC[C@H](N2Cc3cc(NC(=O)CCl)ccc3C2=O)C(=O)N1] |
| 1-273 | 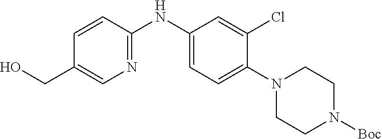 [SMILES: CC(C)(C)OC(=O)N1CCN(c2ccc(Nc3ccc(CO)cn3)cc2Cl)CC1] | 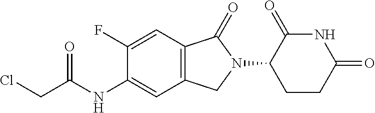 [SMILES: O=C1CC[C@H](N2Cc3cc(NC(=O)CCl)c(F)cc3C2=O)C(=O)N1] |
| 1-274 | 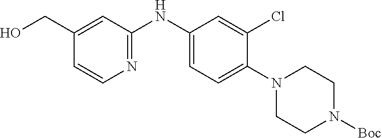 [SMILES: CC(C)(C)OC(=O)N1CCN(c2ccc(Nc3cc(CO)ccn3)cc2Cl)CC1] | 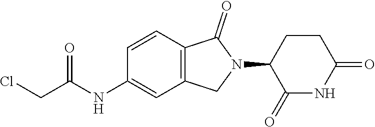 [SMILES: O=C1CC[C@H](N2Cc3cc(NC(=O)CCl)ccc3C2=O)C(=O)N1] |
| 1-275 | 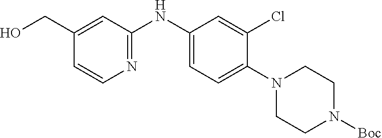 [SMILES: CC(C)(C)OC(=O)N1CCN(c2ccc(Nc3cc(CO)ccn3)cc2Cl)CC1] | 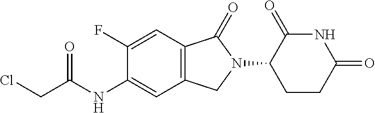 [SMILES: O=C1CC[C@H](N2Cc3cc(NC(=O)CCl)c(F)cc3C2=O)C(=O)N1] |
| 1-276 | 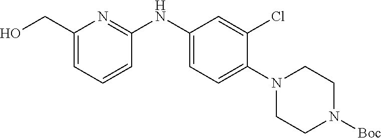 [SMILES: CC(C)(C)OC(=O)N1CCN(c2ccc(Nc3cccc(CO)n3)cc2Cl)CC1] | 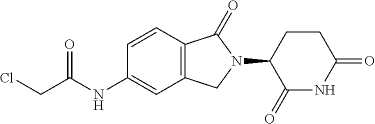 [SMILES: O=C1CC[C@H](N2Cc3cc(NC(=O)CCl)ccc3C2=O)C(=O)N1] |
| 1-277 | 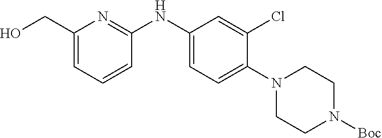 [SMILES: CC(C)(C)OC(=O)N1CCN(c2ccc(Nc3cccc(CO)n3)cc2Cl)CC1] | 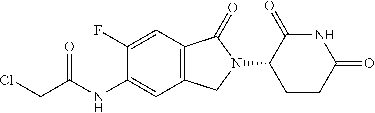 [SMILES: O=C1CC[C@H](N2Cc3cc(NC(=O)CCl)c(F)cc3C2=O)C(=O)N1] |
| 1-278 | 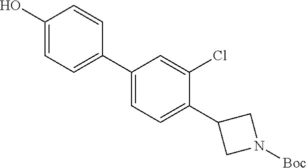 [SMILES: CC(C)(C)OC(=O)N1CC(c2ccc(-c3ccc(O)cc3)cc2Cl)C1] | 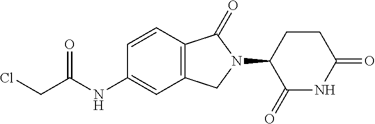 [SMILES: O=C1CC[C@H](N2Cc3cc(NC(=O)CCl)ccc3C2=O)C(=O)N1] |
| 1-279 | 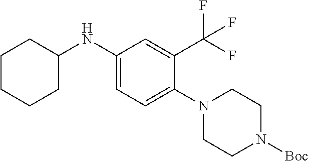 [SMILES: CC(C)(C)OC(=O)N1CCN(c2ccc(NC3CCCCC3)cc2C(F)(F)F)CC1] | 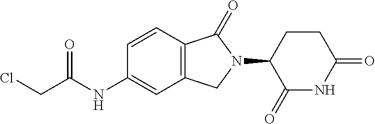 [SMILES: O=C1CC[C@H](N2Cc3cc(NC(=O)CCl)ccc3C2=O)C(=O)N1] |
| 1-280 | 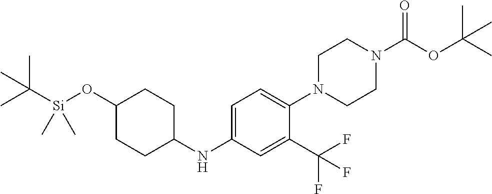 [SMILES: CC(C)(C)OC(=O)N1CCN(c2ccc(NC3CCC(O[Si](C)(C)C(C)(C)C)CC3)cc2C(F)(F)F)CC1] | 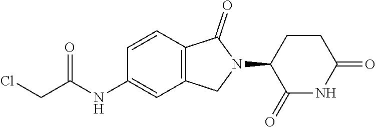 [SMILES: O=C1CC[C@H](N2Cc3cc(NC(=O)CCl)ccc3C2=O)C(=O)N1] |
| 1-281 | 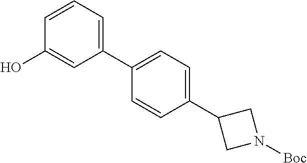 [SMILES: CC(C)(C)OC(=O)N1CC(c2ccc(-c3cccc(O)c3)cc2)C1] | 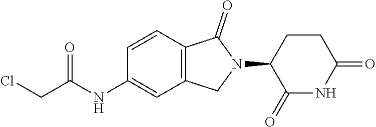 [SMILES: O=C1CC[C@H](N2Cc3cc(NC(=O)CCl)ccc3C2=O)C(=O)N1] |
| 1-282 | 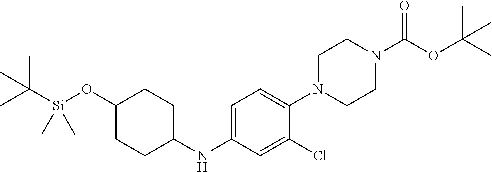 [SMILES: CC(C)(C)OC(=O)N1CCN(c2ccc(NC3CCC(O[Si](C)(C)C(C)(C)C)CC3)cc2Cl)CC1] | 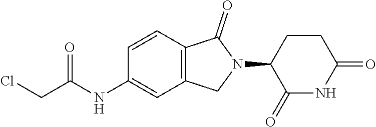 [SMILES: O=C1CC[C@H](N2Cc3cc(NC(=O)CCl)ccc3C2=O)C(=O)N1] |
| 1-283 | 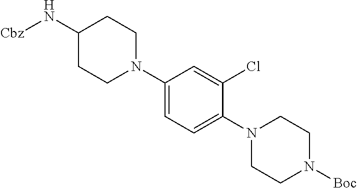 [SMILES: CC(C)(C)OC(=O)N1CCN(c2ccc(N3CCC(NC(=O)OCc4ccccc4)CC3)cc2Cl)CC1] | 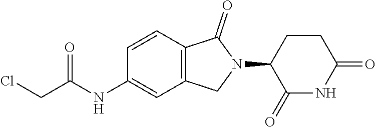 [SMILES: O=C1CC[C@H](N2Cc3cc(NC(=O)CCl)ccc3C2=O)C(=O)N1] |
| 1-284 | 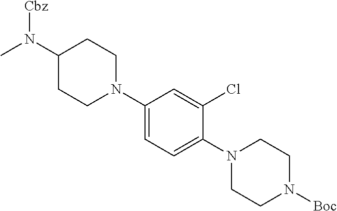 [SMILES: CN(C(=O)OCc1ccccc1)C1CCN(c2ccc(N3CCN(C(=O)OC(C)(C)C)CC3)c(Cl)c2)CC1] | 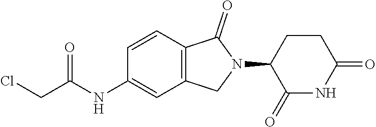 [SMILES: O=C1CC[C@H](N2Cc3cc(NC(=O)CCl)ccc3C2=O)C(=O)N1] |
| 1-285 | 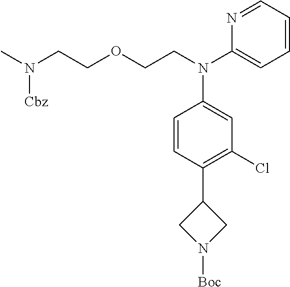 [SMILES: CN(CCOCCN(c1ccc(C2CN(C(=O)OC(C)(C)C)C2)c(Cl)c1)c1ccccn1)C(=O)OCc1ccccc1] | 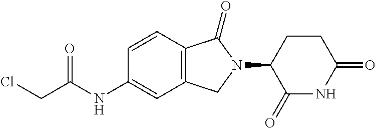 [SMILES: O=C1CC[C@H](N2Cc3cc(NC(=O)CCl)ccc3C2=O)C(=O)N1] |
| 1-286 | 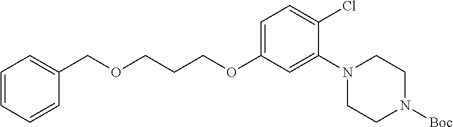 [SMILES: CC(C)(C)OC(=O)N1CCN(c2cc(OCCCOCc3ccccc3)ccc2Cl)CC1] | 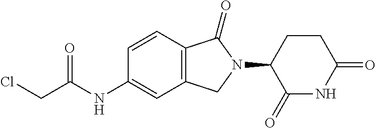 [SMILES: O=C1CC[C@H](N2Cc3cc(NC(=O)CCl)ccc3C2=O)C(=O)N1] |
| 1-287 | 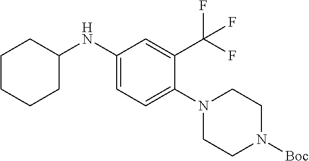 [SMILES: CC(C)(C)OC(=O)N1CCN(c2ccc(NC3CCCCC3)cc2C(F)(F)F)CC1] | 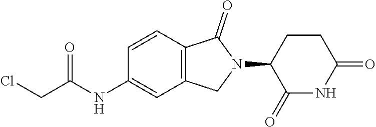 [SMILES: O=C1CC[C@H](N2Cc3cc(NC(=O)CCl)ccc3C2=O)C(=O)N1] |
| 1-288 | 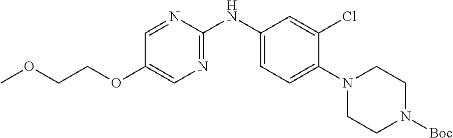 [SMILES: COCCOc1cnc(Nc2ccc(N3CCN(C(=O)OC(C)(C)C)CC3)c(Cl)c2)nc1] | 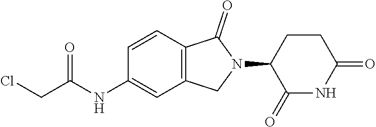 [SMILES: O=C1CC[C@H](N2Cc3cc(NC(=O)CCl)ccc3C2=O)C(=O)N1] |
| 1-289 | 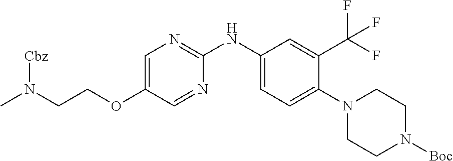 [SMILES: CN(CCOc1cnc(Nc2ccc(N3CCN(C(=O)OC(C)(C)C)CC3)c(C(F)(F)F)c2)nc1)C(=O)OCc1ccccc1] | 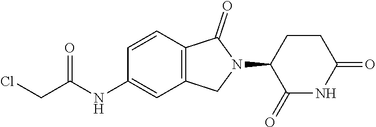 [SMILES: O=C1CC[C@H](N2Cc3cc(NC(=O)CCl)ccc3C2=O)C(=O)N1] |
| 1-290 | 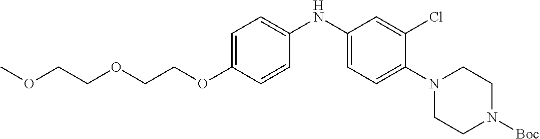 [SMILES: COCCOCCOc1ccc(Nc2ccc(N3CCN(C(=O)OC(C)(C)C)CC3)c(Cl)c2)cc1] | 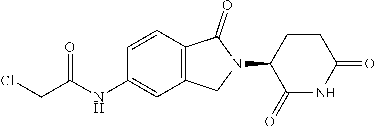 [SMILES: O=C1CC[C@H](N2Cc3cc(NC(=O)CCl)ccc3C2=O)C(=O)N1] |
| 1-291 | 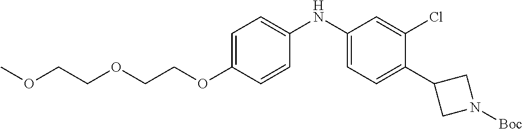 [SMILES: COCCOCCOc1ccc(Nc2ccc(C3CN(C(=O)OC(C)(C)C)C3)c(Cl)c2)cc1] | 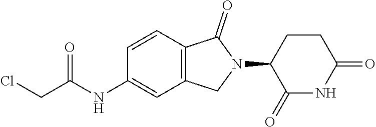 [SMILES: O=C1CC[C@H](N2Cc3cc(NC(=O)CCl)ccc3C2=O)C(=O)N1] |
| 1-292 | 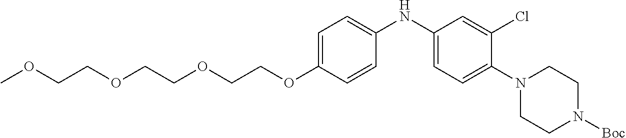 [SMILES: COCCOCCOCCOc1ccc(Nc2ccc(N3CCN(C(=O)OC(C)(C)C)CC3)c(Cl)c2)cc1] | 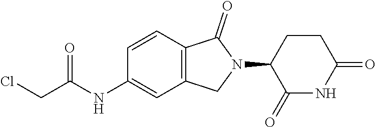 [SMILES: O=C1CC[C@H](N2Cc3cc(NC(=O)CCl)ccc3C2=O)C(=O)N1] |
| 1-293 | 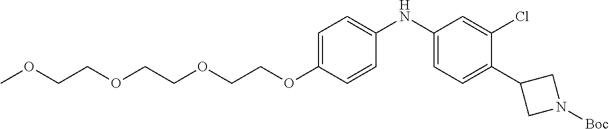 [SMILES: COCCOCCOCCOc1ccc(Nc2ccc(C3CN(C(=O)OC(C)(C)C)C3)c(Cl)c2)cc1] | 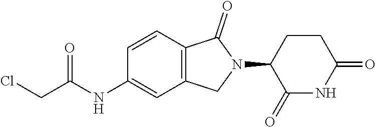 [SMILES: O=C1CC[C@H](N2Cc3cc(NC(=O)CCl)ccc3C2=O)C(=O)N1] |
| 1-294 | 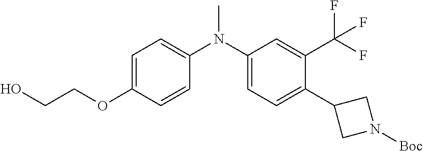 [SMILES: CN(c1ccc(OCCO)cc1)c1ccc(C2CN(C(=O)OC(C)(C)C)C2)c(C(F)(F)F)c1] | 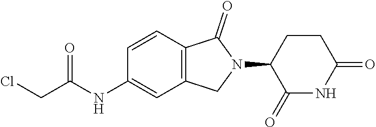 [SMILES: O=C1CC[C@H](N2Cc3cc(NC(=O)CCl)ccc3C2=O)C(=O)N1] |

| Cmpd | Product 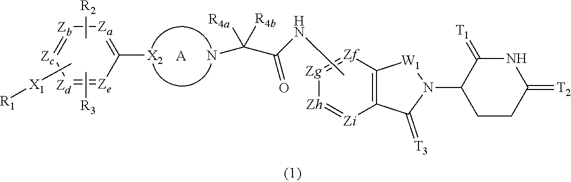 | MS found [M + H]+ |
|---|---|---|
| 1-1 | 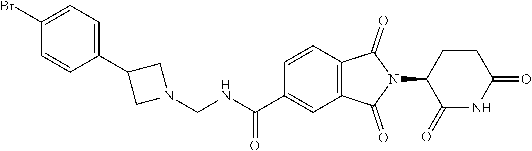 [SMILES: O=C1CC[C@H](N2C(=O)c3ccc(C(=O)NCN4CC(c5ccc(Br)cc5)C4)cc3C2=O)C(=O)N1] | 511.2 513.2 |
| 1-2 | 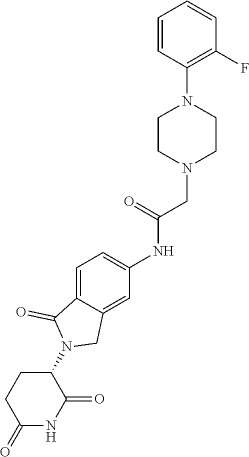 [SMILES: O=C1CC[C@H](N2Cc3cc(NC(=O)CN4CCN(c5ccccc5F)CC4)ccc3C2=O)C(=O)N1] | 480.3 |
| 1-3 | 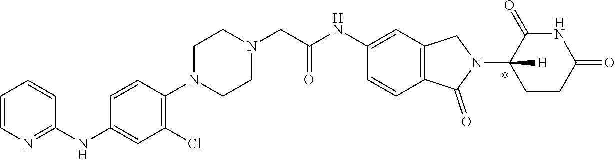 | 492.3 |
| 1-4 | 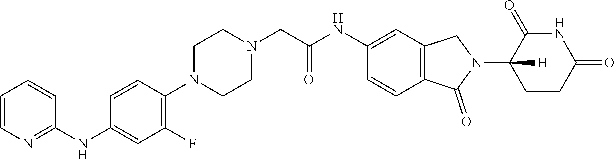 | 584.4 |
| 1-5 | 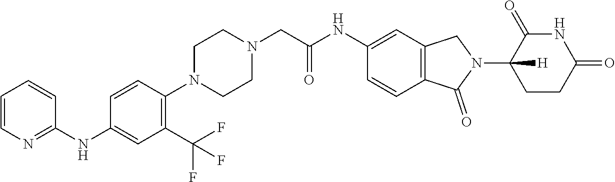 | 622.4 |
| 1-6 | 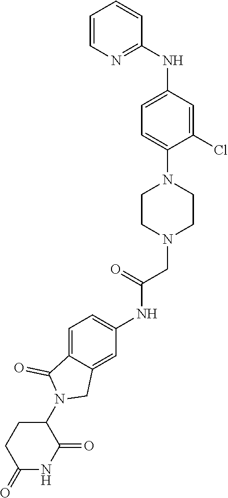 [SMILES: O=C1CCC(N2Cc3cc(NC(=O)CN4CCN(c5ccc(Nc6ccccn6)cc5Cl)CC4)ccc3C2=O)C(=O)N1] | 588.4 |
| 1-7 | 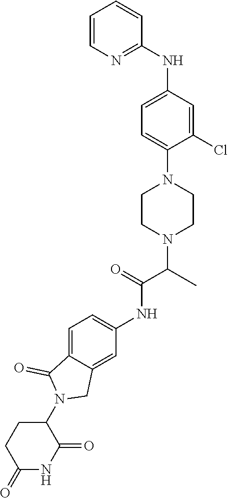 [SMILES: CC(C(=O)Nc1ccc2c(c1)CN(C1CCC(=O)NC1=O)C2=O)N1CCN(c2ccc(Nc3ccccn3)cc2Cl)CC1] | 602.4 |
| 1-8 | 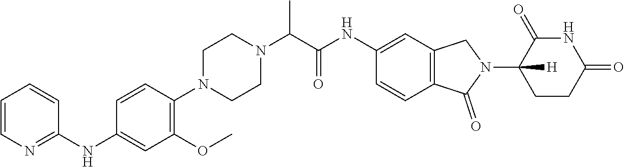 | 598.4 |
| 1-9 | 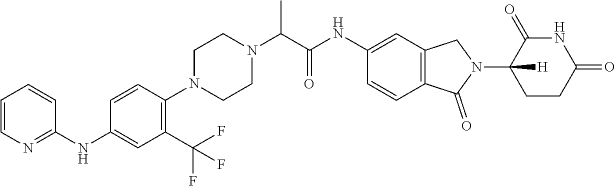 | 636.4 |
| 1-10 | 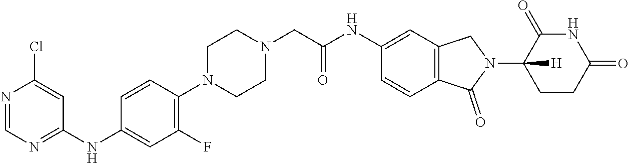 | 607.3 |
| 1-11 | 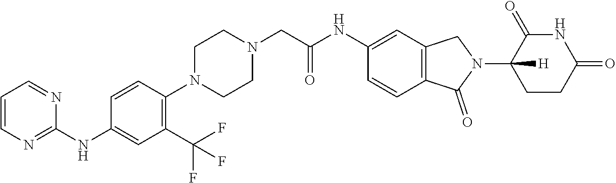 | 623.4 |
| 1-12 | 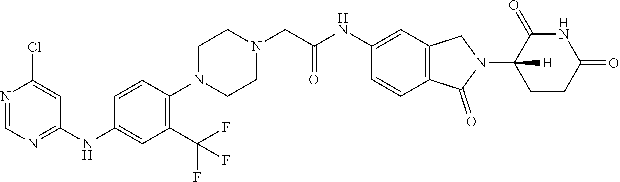 | 657.3 |
| 1-13 | 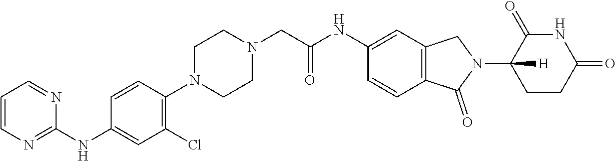 | 589.3 |
| 1-14 | 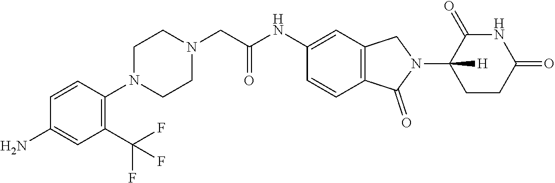 | 545.3 |
| 1-15 | 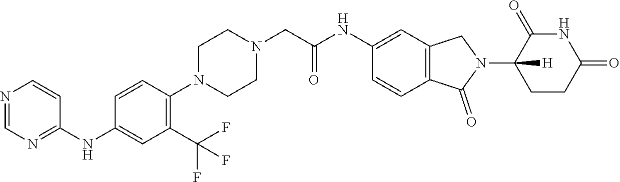 | 623.3 |
| 1-16 | 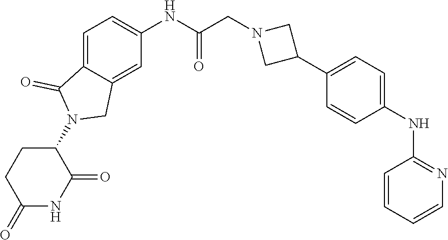 [SMILES: O=C1CC[C@H](N2Cc3cc(NC(=O)CN4CC(c5ccc(Nc6ccccn6)cc5)C4)ccc3C2=O)C(=O)N1] | 525.3 |
| 1-17 | 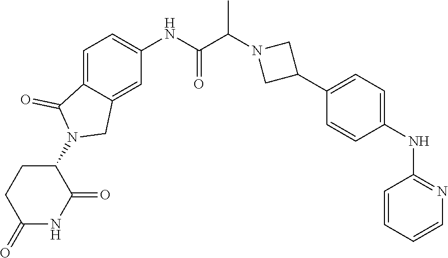 [SMILES: CC(C(=O)Nc1ccc2c(c1)CN([C@H]1CCC(=O)NC1=O)C2=O)N1CC(c2ccc(Nc3ccccn3)cc2)C1] | 539.3 |
| 1-18 | 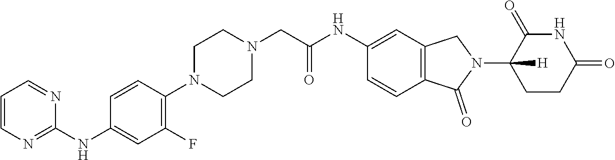 | 585.4 |
| 1-19 | 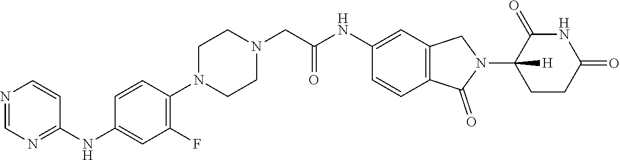 | 589.3 |
| 1-20 | 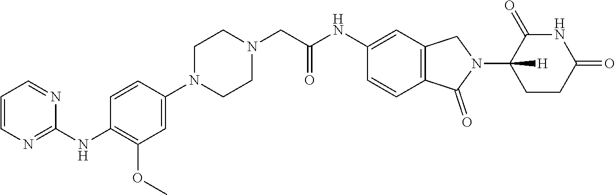 | 585.4 |
| 1-21 | 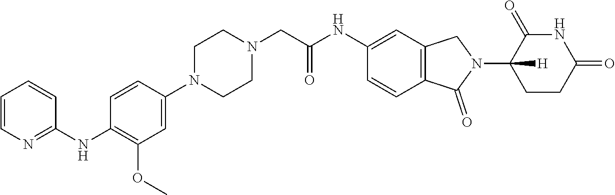 | 584.4 |
| 1-22 | 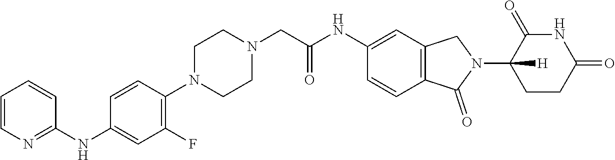 | 572.4 |
| 1-23 | 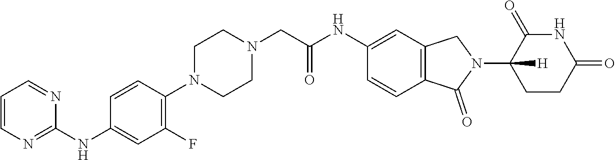 | 573.4 |
| 1-24 | 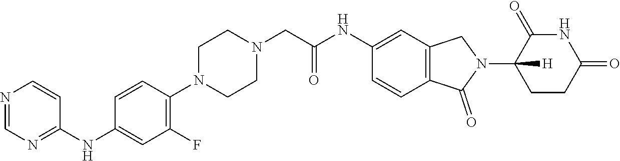 | 573.4 |
| 1-25 | 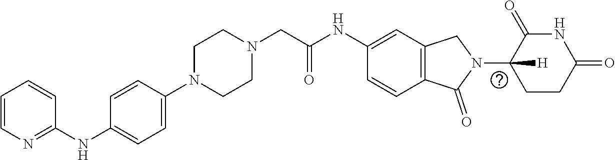 | 554.4 |
| 1-26 | 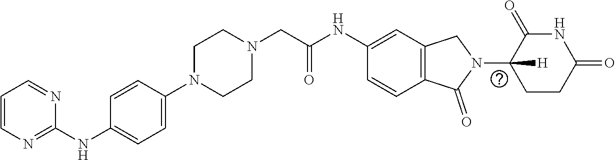 | 555.4 |
| 1-27 | 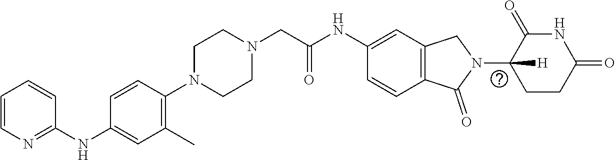 | 568.4 |
| 1-28 | 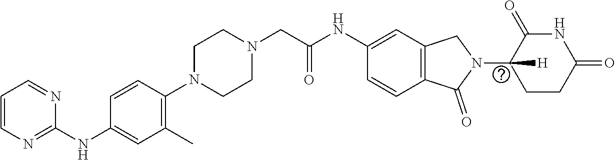 | 569.4 |
| 1-29 | 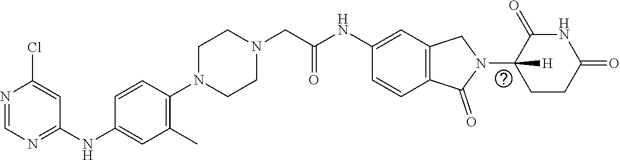 | 603.4 |
| 1-30 | 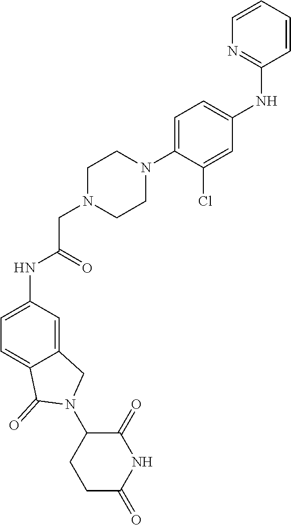 [SMILES: O=C1CCC(N2Cc3cc(NC(=O)CN4CCN(c5ccc(Nc6ccccn6)cc5Cl)CC4)ccc3C2=O)C(=O)N1] | 602.4 |
| 1-31 | 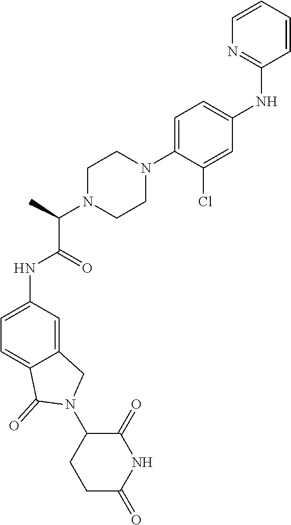 [SMILES: C[C@H](C(=O)Nc1ccc2c(c1)CN(C1CCC(=O)NC1=O)C2=O)N1CCN(c2ccc(Nc3ccccn3)cc2Cl)CC1] | 602.4 |
| 1-32 | 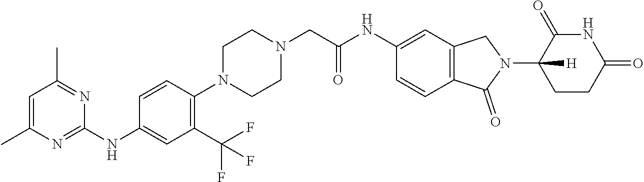 | 651.4 |
| 1-33 | 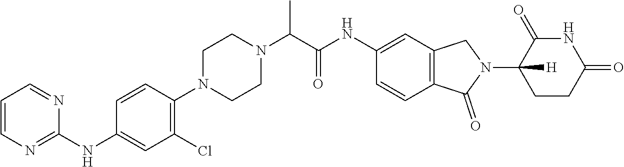 | 603.4 |
| 1-34 | 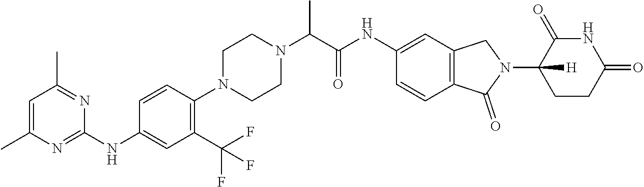 | 665.4 |
| 1-35 | 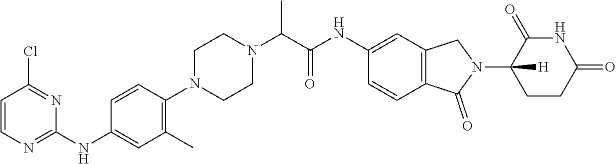 | 617.4 |
| 1-36 | 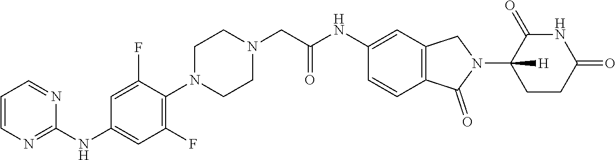 | 591.3 |
| 1-37 | 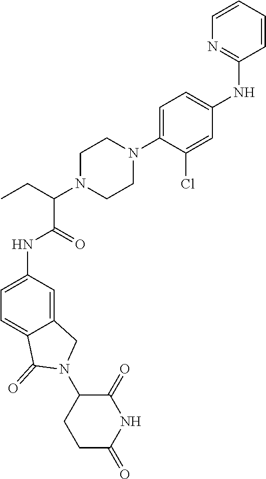 [SMILES: CCC(C(=O)Nc1ccc2c(c1)CN(C1CCC(=O)NC1=O)C2=O)N1CCN(c2ccc(Nc3ccccn3)cc2Cl)CC1] | 616.4 |
| 1-38 | 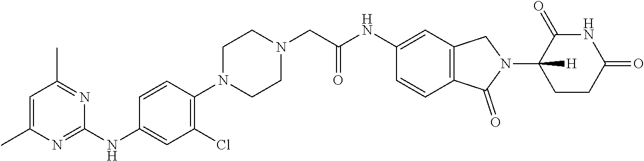 | 617.4 |
| 1-39 | 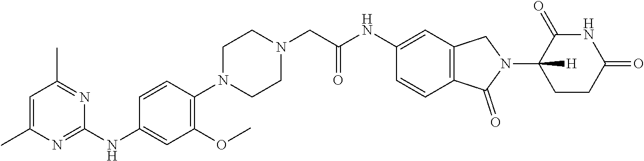 | 613.4 |
| 1-40 | 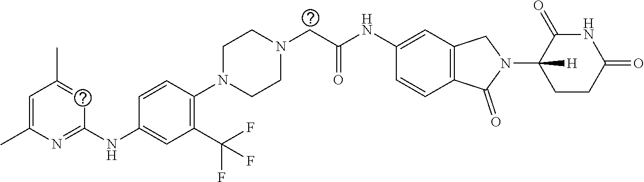 | 665.4 |
| 1-41 | 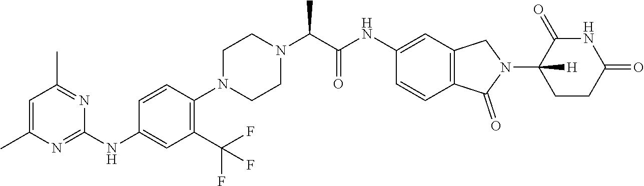 | 665.3 |
| 1-42 | 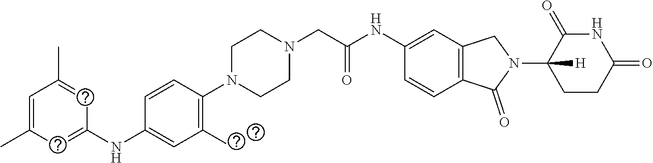 | 608.4 |
| 1-43 | 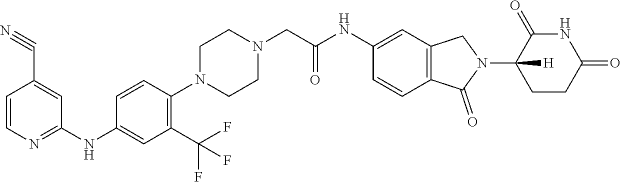 | 647.4 |
| 1-44 | 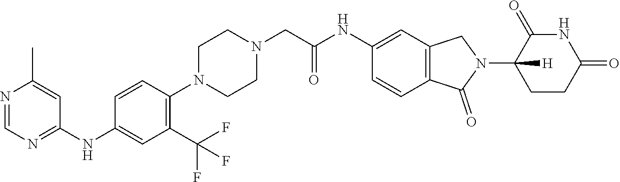 | 637.4 |
| 1-45 | 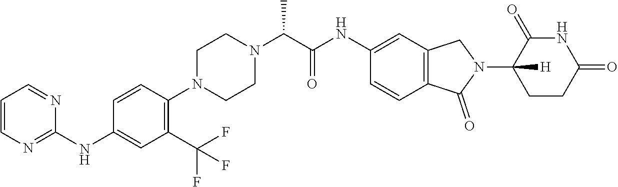 | 637.4 |
| 1-46 | 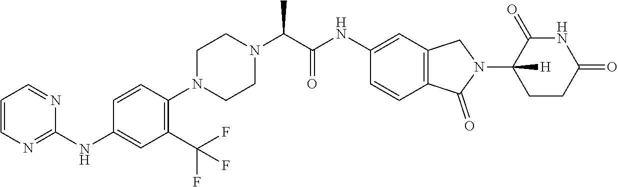 | 637.3 |
| 1-47 | 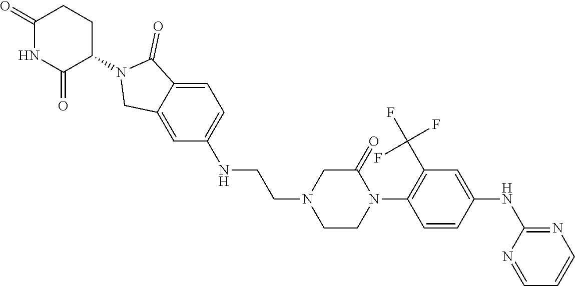 [SMILES: O=C1CC[C@H](N2Cc3cc(NCCN4CCN(c5ccc(Nc6ncccn6)cc5C(F)(F)F)C(=O)C4)ccc3C2=O)C(=O)N1] | 637.4 |
| 1-48 | 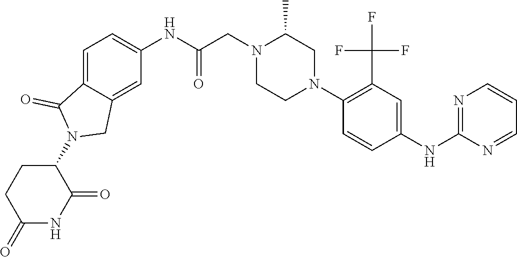 [SMILES: C[C@@H]1CN(c2ccc(Nc3ncccn3)cc2C(F)(F)F)CCN1CC(=O)Nc1ccc2c(c1)CN([C@H]1CCC(=O)NC1=O)C2=O] | 637.4 |
| 1-49 | 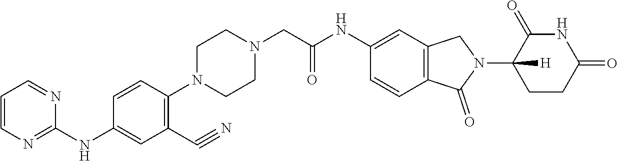 | 580.4 |
| 1-50 | 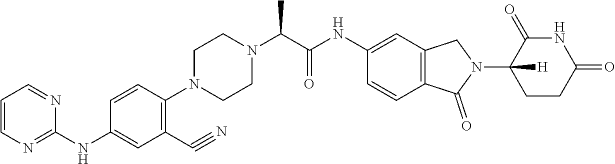 | 594.4 |
| 1-51 | 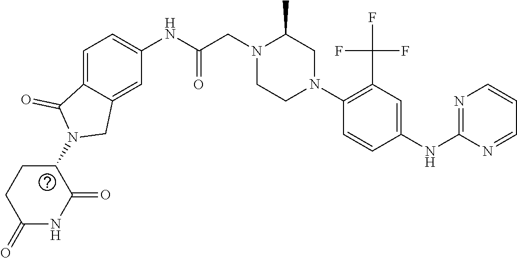 [SMILES: C[C@H]1CN(c2ccc(Nc3ncccn3)cc2C(F)(F)F)CCN1CC(=O)Nc1ccc2c(c1)CN([C@H]1CCC(=O)NC1=O)C2=O] | 637.4 |
| 1-52 | 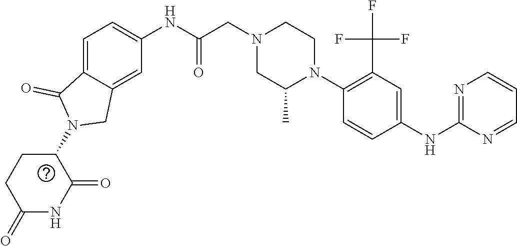 [SMILES: C[C@@H]1CN(CC(=O)Nc2ccc3c(c2)CN([C@H]2CCC(=O)NC2=O)C3=O)CCN1c1ccc(Nc2ncccn2)cc1C(F)(F)F] | 637.4 |
| 1-53 | 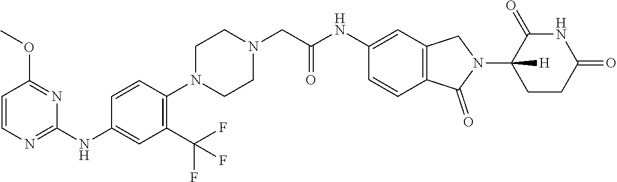 | 653.4 |
| 1-54 | 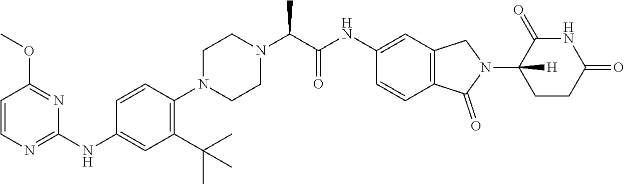 | 667.4 |
| 1-55 | 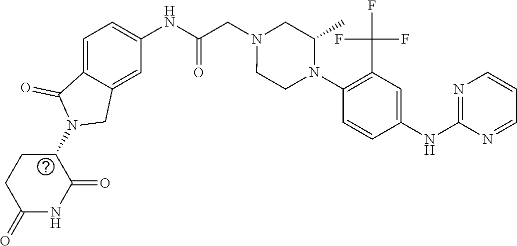 [SMILES: C[C@H]1CN(CC(=O)Nc2ccc3c(c2)CN([C@H]2CCC(=O)NC2=O)C3=O)CCN1c1ccc(Nc2ncccn2)cc1C(F)(F)F] | 637.3 |
| 1-56 | 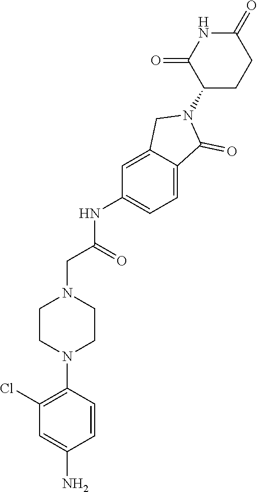 [SMILES: Nc1ccc(N2CCN(CC(=O)Nc3ccc4c(c3)CN([C@H]3CCC(=O)NC3=O)C4=O)CC2)c(Cl)c1] | 511.2 |
| | ¹H NMR (400 MHz, DMSO-d6) δ ppm 1.90-2.01 (m, | |
| | 1H), 2.33-2.41 (m, 1H), 2.52-2.63 (m, 2H), 2.67 (s, | |
| | 4H), 2.90 (s, 4H), 3.24 (s, 2H), 4.30 (d, J = 17.4 Hz, | |
| | 1H), 4.44 (d, J = 17.2 Hz, 1H), 5.09 (dd, J = 13.6, 5.0 | |
| | Hz, 1H), 6.47 (dd, J = 8.8, 2.6 Hz, 1H), 6.58 (d, J = 2.6 | |
| | Hz, 1H), 7.00 (d, J = 8.8 Hz, 1H), 7.63-7.73 (m, 2H), | |
| | 8.02 (s, 1H), 8.16 (s, 1H), 10.08 (s, 1H), 10.98 (s, 1H) | |
| 1-57 | 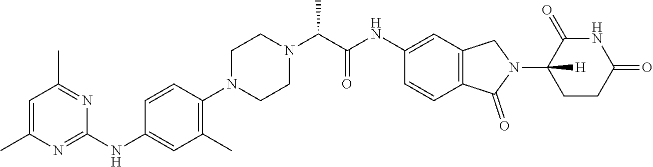 | 597.4 |
| 1-58 | 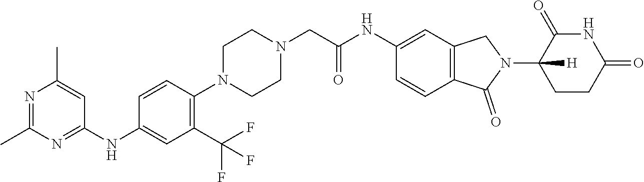 | 651.4 |
| 1-59 | 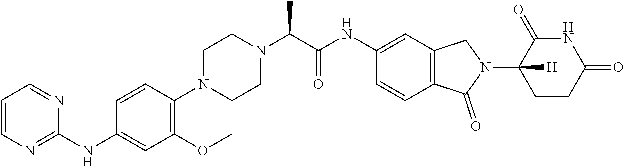 | 599.4 |
| 1-60 | 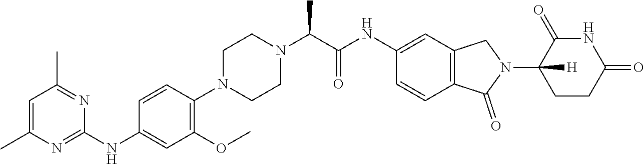 | 627.4 |
| 1-61 | 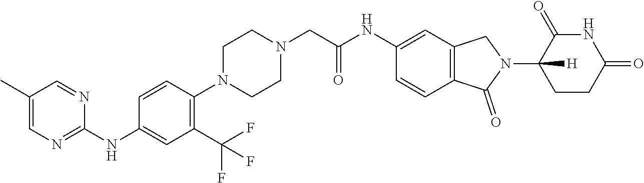 | 637.3 |
| 1-62 | 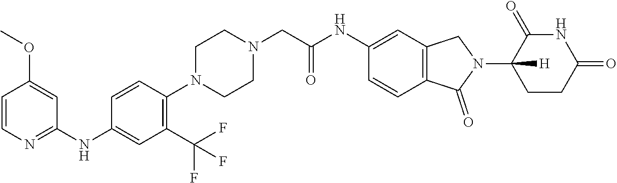 | 652.4 |
| 1-63 | 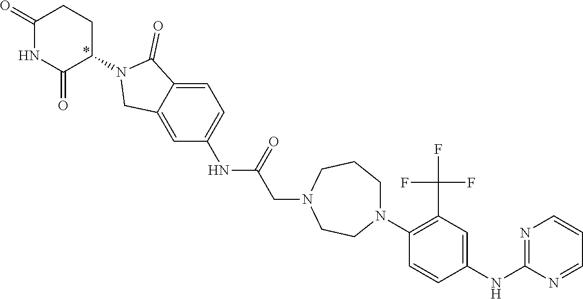 [SMILES: O=C1CC[C@H](N2Cc3cc(NC(=O)CN4CCCN(c5ccc(Nc6ncccn6)cc5C(F)(F)F)CC4)ccc3C2=O)C(=O)N1] | 637.3 |
| 1-64 | 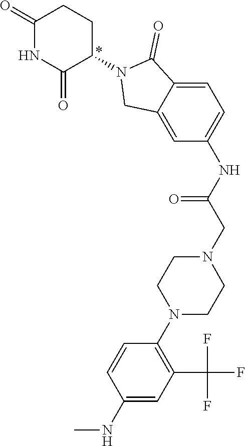 [SMILES: CNc1ccc(N2CCN(CC(=O)Nc3ccc4c(c3)CN([C@H]3CCC(=O)NC3=O)C4=O)CC2)c(C(F)(F)F)c1] | 559.2 |
| 1-65 | 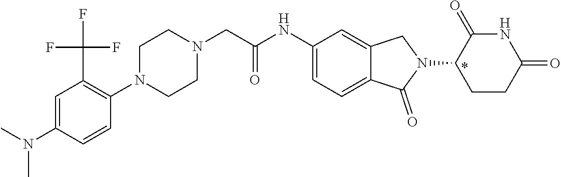 [SMILES: CN(C)c1ccc(N2CCN(CC(=O)Nc3ccc4c(c3)CN([C@H]3CCC(=O)NC3=O)C4=O)CC2)c(C(F)(F)F)c1] | 573.3 |
| | ¹H NMR (400 MHz, DMSO-d6) δ ppm 1.97-2.03 (m, | |
| | 1 H), 2.32-2.43 (m, 1 H), 2.57-2.64 (m, 5 H), 2.76- | |
| | 2.99 (m, 11 H), 3.23 (br s, 2 H), 4.37 (dd, J = 56.0, 17.2 | |
| | Hz, 2 H), 5.09 (dd, J = 13.6, 5.2 Hz, 1 H), 6.84 (d, J = | |
| | 2.8 Hz, 1 H), 6.97 (dd, J = 9.2, 2.8 Hz, 1 H), 7.45 (d, J = | |
| | 8.8 Hz, 1 H), 7.66-7.71 (m, 2 H), 8.03 (br s, 1 H), | |
| | 10.10 (br s, 1 H), 10.98 (br s, 1 H) | |
| 1-66 | 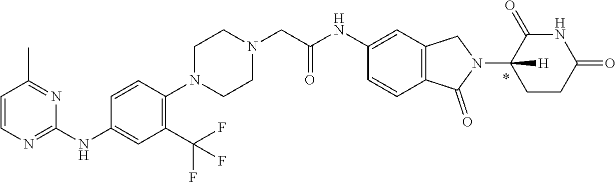 | 637.4 |
| 1-67 | 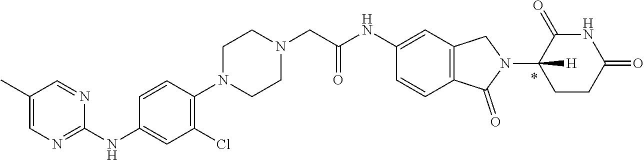 | 603.4 |
| 1-68 | 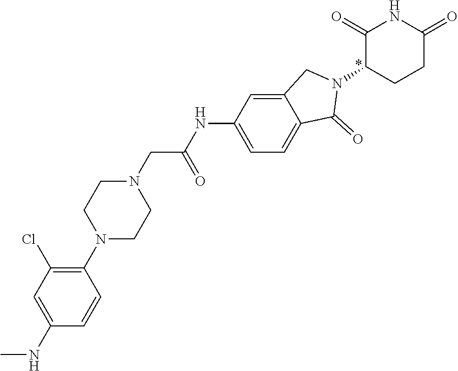 [SMILES: CNc1ccc(N2CCN(CC(=O)Nc3ccc4c(c3)CN([C@H]3CCC(=O)NC3=O)C4=O)CC2)c(Cl)c1] | 525.3 |
| 1-69 | 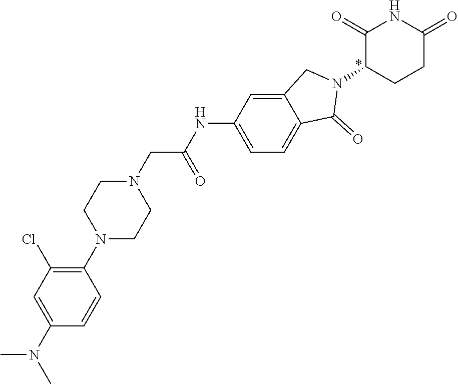 [SMILES: CN(C)c1ccc(N2CCN(CC(=O)Nc3ccc4c(c3)CN([C@H]3CCC(=O)NC3=O)C4=O)CC2)c(Cl)c1] | 539.3 |
| | ¹H NMR (400 MHz, DMSO-d6) δ ppm 1.94-2.06 (m, | |
| | 1H), 2.27-2.41 (m, 1H), 2.52-2.58 (m, 1H), 2.68 (s, | |
| | 4H), 2.83 (s, 6H), 2.90 (s, 4H), 3.24 (s, 3H), 4.29 (d, J = | |
| | 17.0 Hz, 1H), 4.43 (d, J = 17.2 Hz, 1H), 5.08 (d, J = | |
| | 8.6 Hz, 1H), 6.55-6.83 (m, 2H), 7.06 (d, J = 8.6 Hz, | |
| | 1H), 7.68 (s, 2H), 8.02 (s, 1H), 8.29 (s, 1H), 10.10 (s, | |
| | 1H), 10.98 (s, 1H) | |
| 1-70 | 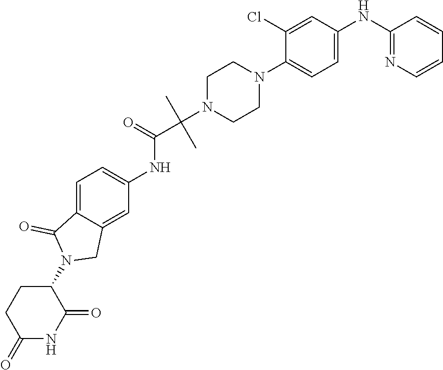 [SMILES: CC(C)(C(=O)Nc1ccc2c(c1)CN([C@H]1CCC(=O)NC1=O)C2=O)N1CCN(c2ccc(Nc3ccccn3)cc2Cl)CC1] | 616.9 |
| 1-71 | 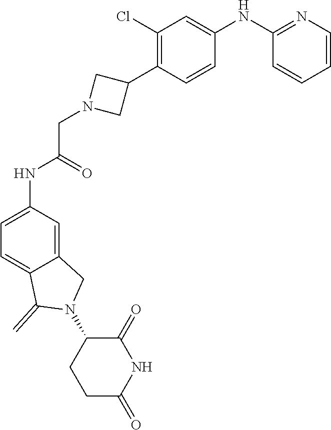 [SMILES: C=C1c2ccc(NC(=O)CN3CC(c4ccc(Nc5ccccn5)cc4Cl)C3)cc2CN1[C@H]1CCC(=O)NC1=O] | 559.3 |
| 1-72 | 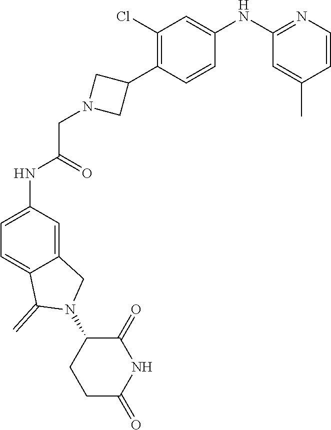 [SMILES: C=C1c2ccc(NC(=O)CN3CC(c4ccc(Nc5cc(C)ccn5)cc4Cl)C3)cc2CN1[C@H]1CCC(=O)NC1=O] | 573.2 |
| 1-73 | 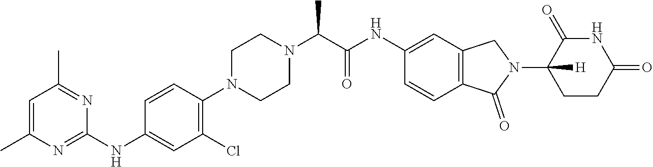 | 631.4 |
| 1-74 | 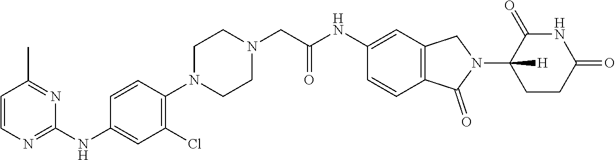 | 603.2 |
| 1-75 | 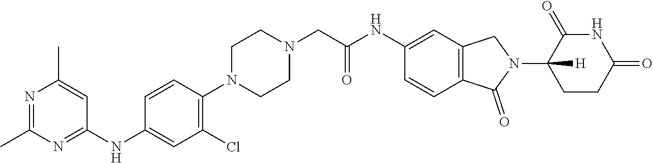 | 617.5 |
| 1-76 | 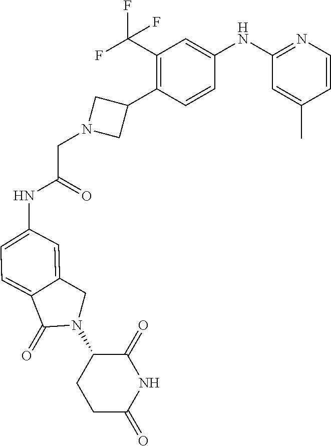 [SMILES: Cc1ccnc(Nc2ccc(C3CN(CC(=O)Nc4ccc5c(c4)CN([C@H]4CCC(=O)NC4=O)C5=O)C3)c(C(F)(F)F)c2)c1] | 607.4 |
| 1-77 | 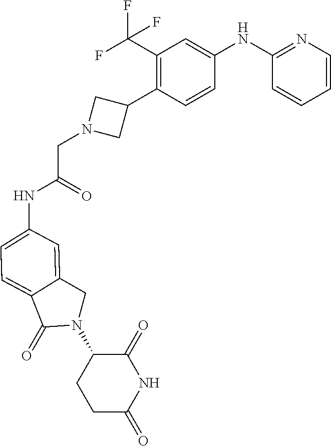 [SMILES: O=C1CC[C@H](N2Cc3cc(NC(=O)CN4CC(c5ccc(Nc6ccccn6)cc5C(F)(F)F)C4)ccc3C2=O)C(=O)N1] | 593.3 |
| 1-78 | 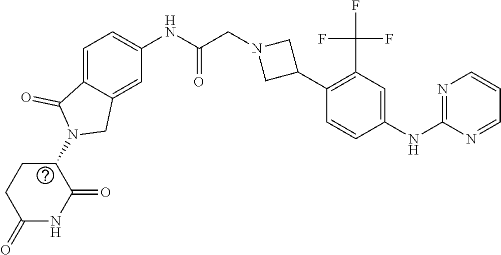 [SMILES: O=C1CC[C@H](N2Cc3cc(NC(=O)CN4CC(c5ccc(Nc6ncccn6)cc5C(F)(F)F)C4)ccc3C2=O)C(=O)N1] | 594.3 |
| 1-79 | 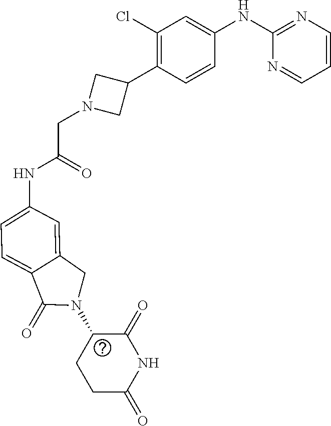 [SMILES: O=C1CC[C@H](N2Cc3cc(NC(=O)CN4CC(c5ccc(Nc6ncccn6)cc5Cl)C4)ccc3C2=O)C(=O)N1] | 560.3 |
| 1-80 | 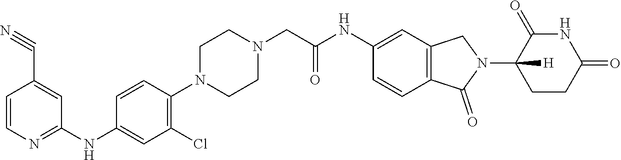 | 613.4 |
| 1-81 | 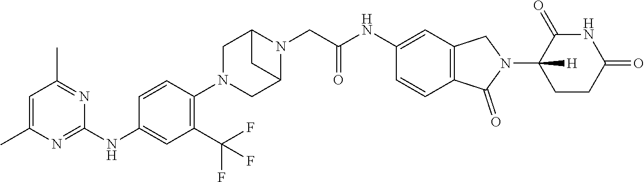 | 663.4 |
| 1-82 | 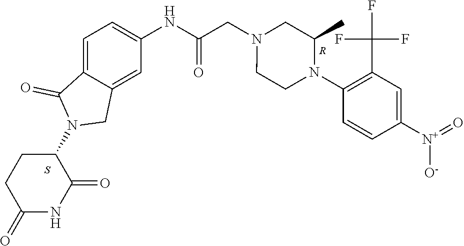 | 589.3 |
| 1-83 | 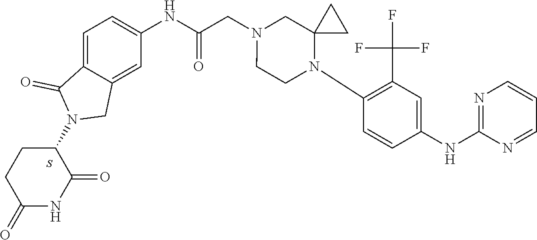 | 649.4 |
| 1-84 | 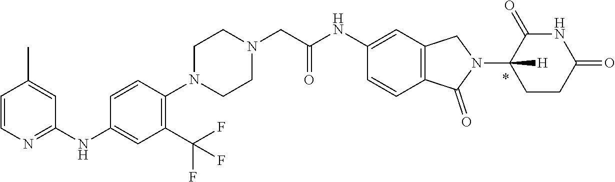 | 636.4 |
| 1-85 | 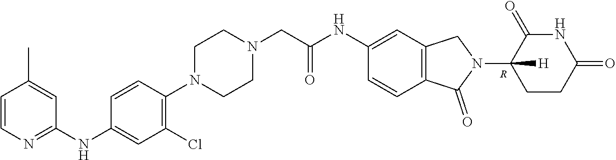 | 602.4 |
| 1-86 | 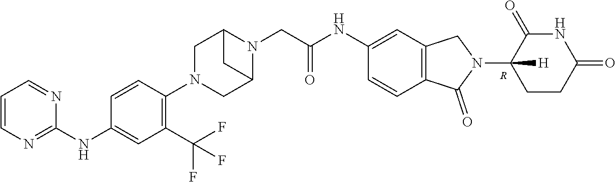 | 635.4 |
| 1-87 | 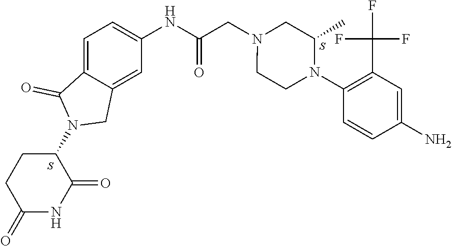 | 559.3 |
| 1-88 | 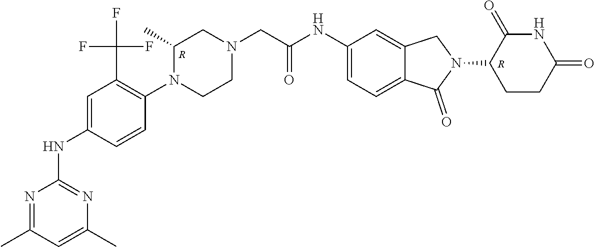 | 665.4 |
| 1-89 | 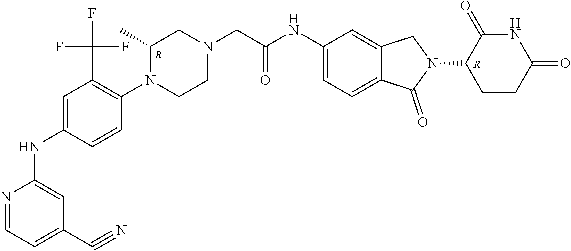 | 661.4 |
| 1-90 | 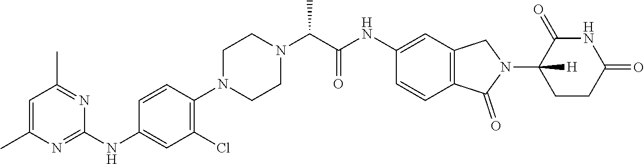 | 631.4 |
| 1-91 | 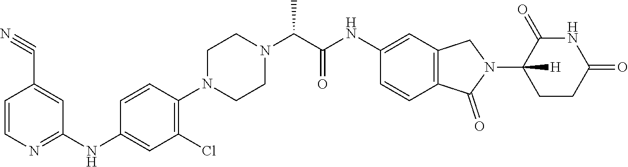 | 627.4 |
| 1-92 | 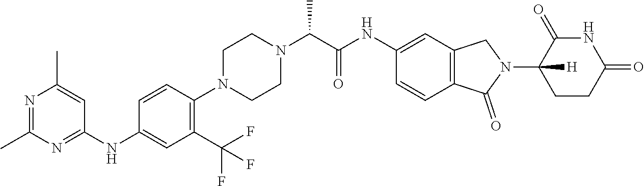 | 665.4 |
| 1-93 | 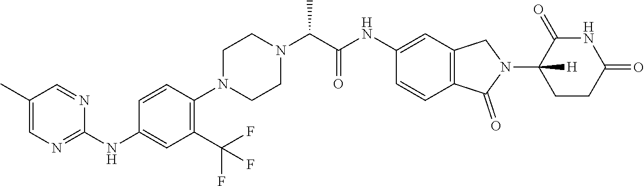 | 651.4 |
| 1-94 | 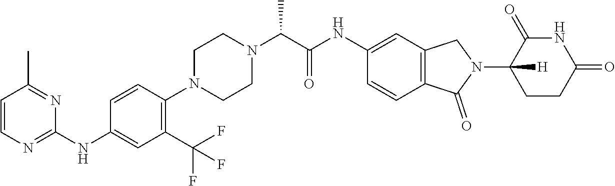 | 651.3 |
| 1-95 | 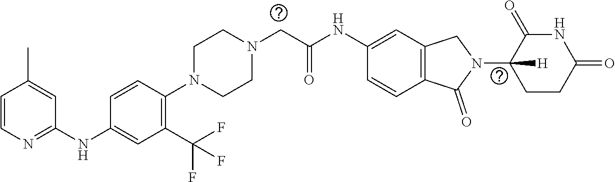 | 650.4 |
| 1-96 | 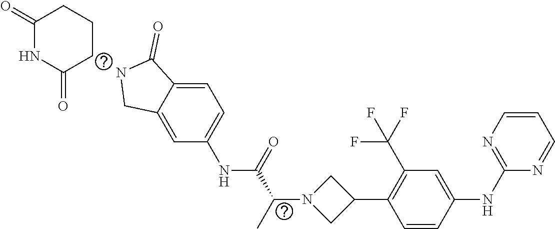 | 608.4 |
| 1-97 | 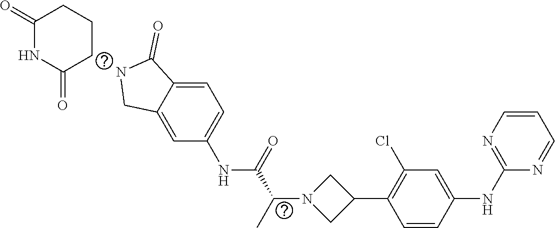 | 574.3 |
| 1-98 | 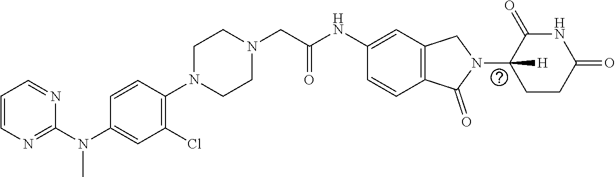 | 603.4 |
| 1-99 | 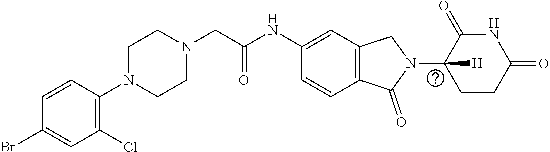 | 574.2 576.2 |
| 1-100 | 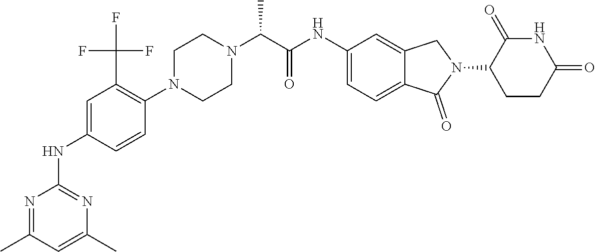 | 679.5 |
| 1-101 | 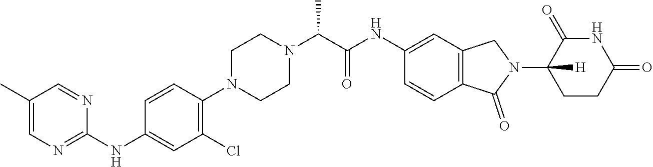 | 617.4 |
| 1-102 | 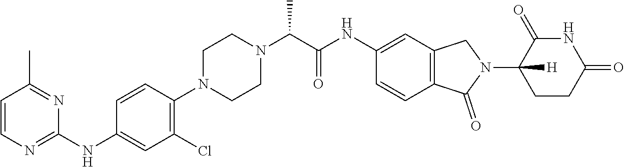 | 617.4 |
| 1-103 | 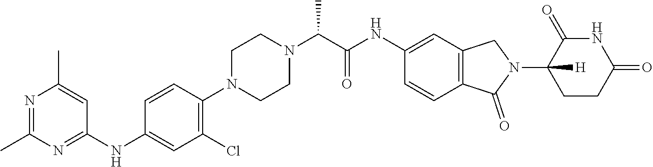 | 631.4 |
| 1-104 | 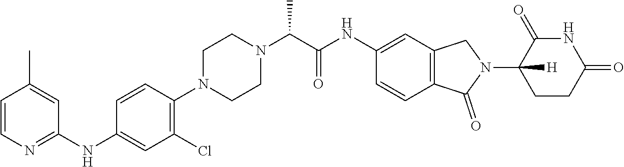 | 616.4 |
| 1-105 | 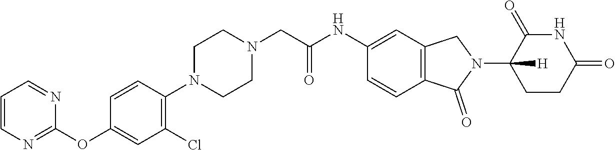 | 509.3 |
| 1-106 | 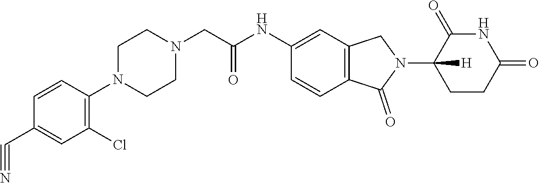 | 521.2 |
| 1-107 | 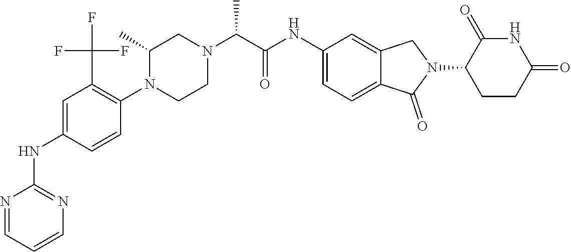 [SMILES: C[C@H](C(=O)Nc1ccc2c(c1)CN([C@H]1CCC(=O)NC1=O)C2=O)N1CCN(c2ccc(Nc3ncccn3)cc2C(F)(F)F)[C@H](C)C1] | 651.4 |
| 1-108 | 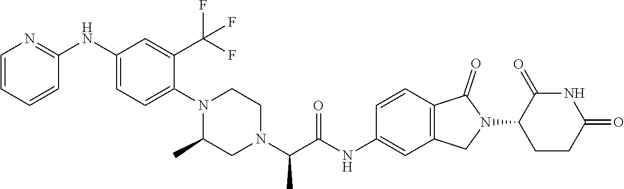 [SMILES: C[C@H](C(=O)Nc1ccc2c(c1)CN([C@H]1CCC(=O)NC1=O)C2=O)N1CCN(c2ccc(Nc3ccccn3)cc2C(F)(F)F)[C@H](C)C1] | 650.4 |
| 1-109 | 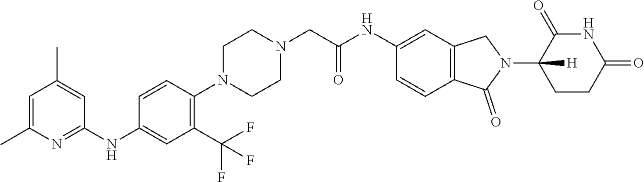 | 650.4 |
| 1-110 | 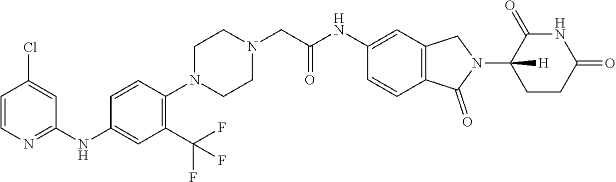 | 656.3 |
| 1-111 | 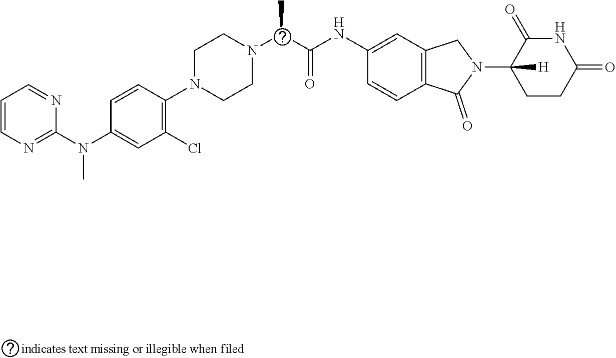 | 617.4 |
| 1-112 | 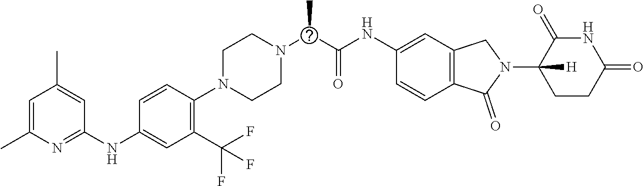 | 664.4 |
| 1-113 | 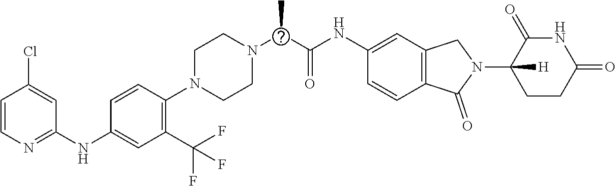 | 670.3 |
| 1-114 | 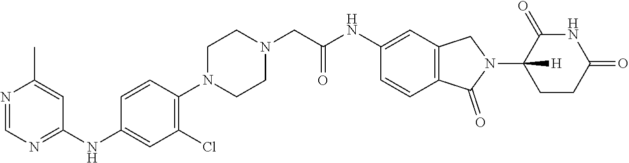 | 603.4 |
| 1-115 | 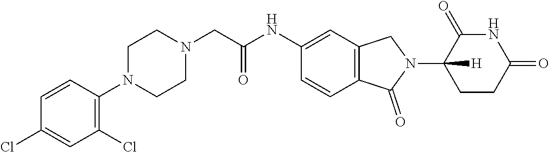 | 530.2 |
| 1-116 | 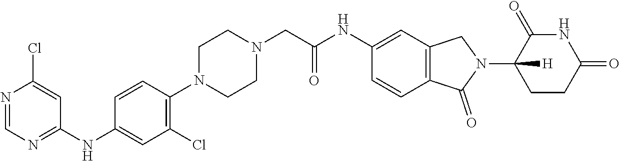 | 623.3 |
| 1-117 | 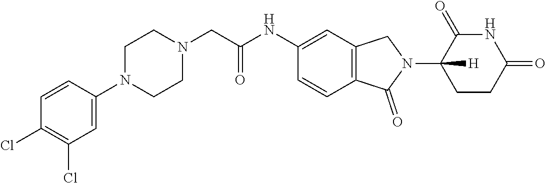 | 530.2 |
| 1-118 | 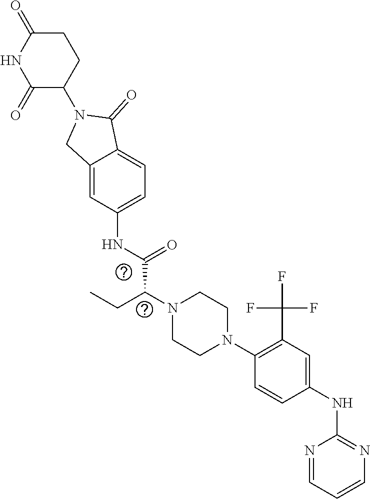 [SMILES: CC[C@H](C(=O)Nc1ccc2c(c1)CN(C1CCC(=O)NC1=O)C2=O)N1CCN(c2ccc(Nc3ncccn3)cc2C(F)(F)F)CC1] | 651.4 |
| 1-119 | 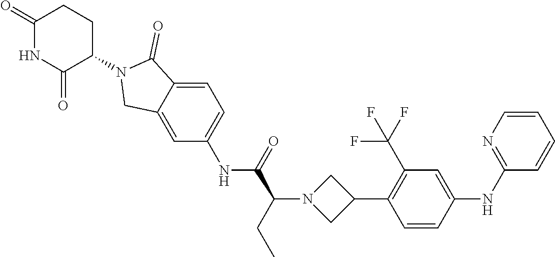 [SMILES: CC[C@@H](C(=O)Nc1ccc2c(c1)CN([C@H]1CCC(=O)NC1=O)C2=O)N1CC(c2ccc(Nc3ccccn3)cc2C(F)(F)F)C1] | 621.4 |
| 1-120 | 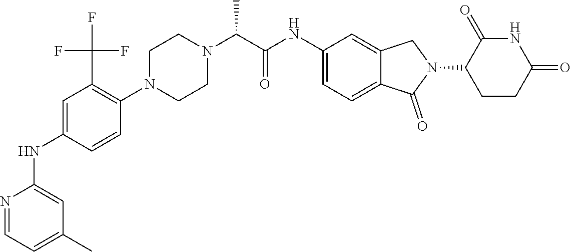 | 664.4 |
| 1-121 | 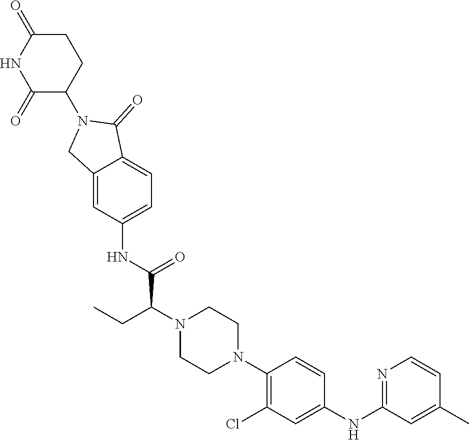 [SMILES: CC[C@@H](C(=O)Nc1ccc2c(c1)CN(C1CCC(=O)NC1=O)C2=O)N1CCN(c2ccc(Nc3cc(C)ccn3)cc2Cl)CC1] | 630.4 |
| 1-122 | 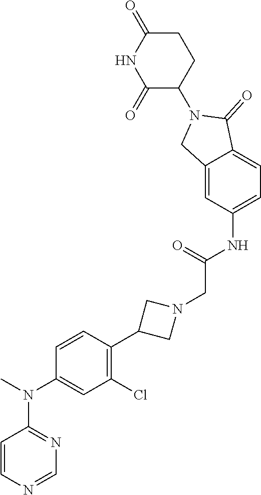 [SMILES: CN(c1ccc(C2CN(CC(=O)Nc3ccc4c(c3)CN(C3CCC(=O)NC3=O)C4=O)C2)c(Cl)c1)c1ccncn1] | 574.3 |
| 1-123 | 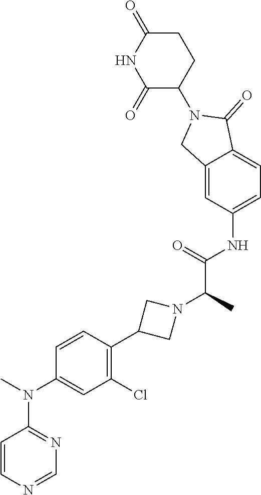 [SMILES: C[C@H](C(=O)Nc1ccc2c(c1)CN(C1CCC(=O)NC1=O)C2=O)N1CC(c2ccc(N(C)c3ccncn3)cc2Cl)C1] | 588.4 |
| 1-124 | 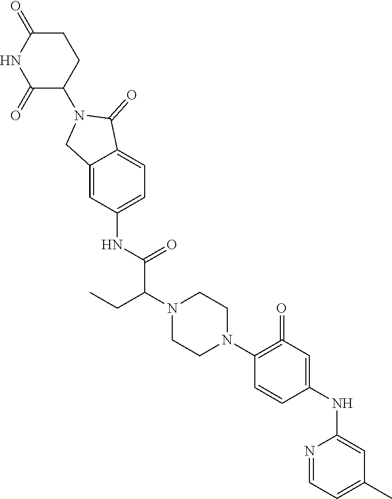 | 630.4 |
| 1-125 | 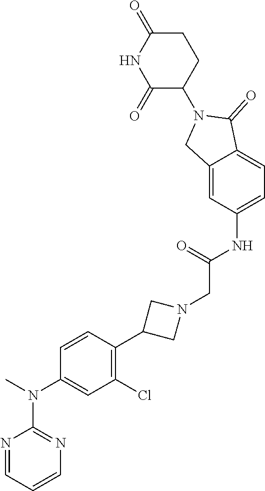 [SMILES: CN(c1ccc(C2CN(CC(=O)Nc3ccc4c(c3)CN(C3CCC(=O)NC3=O)C4=O)C2)c(Cl)c1)c1ncccn1] | 574.3 |
| 1-126 | 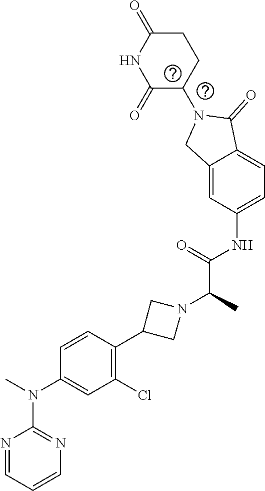 [SMILES: C[C@H](C(=O)Nc1ccc2c(c1)CN(C1CCC(=O)NC1=O)C2=O)N1CC(c2ccc(N(C)c3ncccn3)cc2Cl)C1] | 588.3 |
| 1-127 | 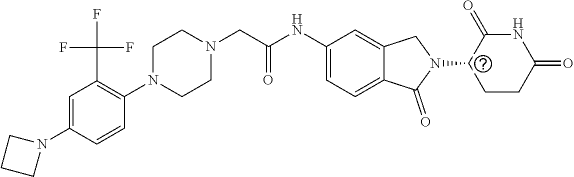 [SMILES: O=C1CC[C@H](N2Cc3cc(NC(=O)CN4CCN(c5ccc(N6CCC6)cc5C(F)(F)F)CC4)ccc3C2=O)C(=O)N1] | 585.3 |
| | ¹H NMR (400 MHz, DMSO-d6) δ ppm 1.95-2.03 (m, | |
| | 1 H), 2.25-2.35 (m, 2 H), 2.35-2.44 (m, 1 H), 2.58- | |
| | 2.57 (m, 5 H), 2.83-2.96 (m, 5 H), 3.23 (br s, 2 H), | |
| | 3.82 (t, J = 7.2 Hz, 4 H), 4.37 (dd, J = 56.4, 17.2 Hz, 2 | |
| | H), 5.09 (dd, J = 13.6, 5.2 Hz, 1 H), 6.55 (d, J = 2.8 Hz, | |
| | 1 H), 6.66 (dd, J = 8.4, 2.4 Hz, 1 H), 7.45 (d, J = 8.6 | |
| | Hz, 1 H), 7.66-7.71 (m, 2 H), 8.03 (br s, 1 H), 10.10 | |
| | (br s, 1 H), 10.98 (br s, 1 H) | |
| 1-128 | 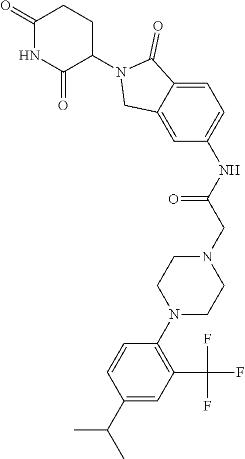 [SMILES: CC(C)c1ccc(N2CCN(CC(=O)Nc3ccc4c(c3)CN(C3CCC(=O)NC3=O)C4=O)CC2)c(C(F)(F)F)c1] | 572.3 |
| | ¹H NMR (400 MHz, CDCl3) δ ppm 1.26 (d, J = 6.8 | |
| | Hz, 6H), 2.18-2.30 (m, 1H), 2.32-2.43 (m, 1H), 2.80 (s, | |
| | 4H), 2.88-2.83 (m, 1H), 2.93 (dd, J = 13.8, 6.6 Hz, 2H), | |
| | 3.00-3.05 (m, 4H), 3.26 (s, 2H), 4.34 (d, J = 16.2 Hz, | |
| | 1H), 4.49 (d, J = 16.2 Hz, 1H), 5.21 (dd, J = 13.2, 5.2 | |
| | Hz, 1H), 7.36-7.30 (m, 2H), 7.40 (d, J = 8.3 Hz, 1H), | |
| | 7.48 (s, 1H), 7.85 (d, J = 8.2 Hz, 1H), 8.04 (s, 1H), 8.22 | |
| | (s, 1H), 9.56 (s, 1H) | |
| 1-129 | 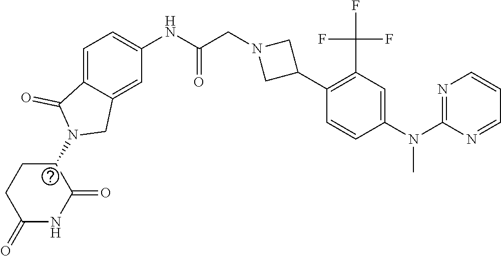 [SMILES: CN(c1ccc(C2CN(CC(=O)Nc3ccc4c(c3)CN([C@H]3CCC(=O)NC3=O)C4=O)C2)c(C(F)(F)F)c1)c1ncccn1] | 608.3 |
| 1-130 | 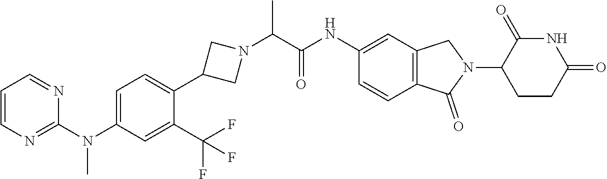 [SMILES: CC(C(=O)Nc1ccc2c(c1)CN(C1CCC(=O)NC1=O)C2=O)N1CC(c2ccc(N(C)c3ncccn3)cc2C(F)(F)F)C1] | 622.4 |
| 1-131 | 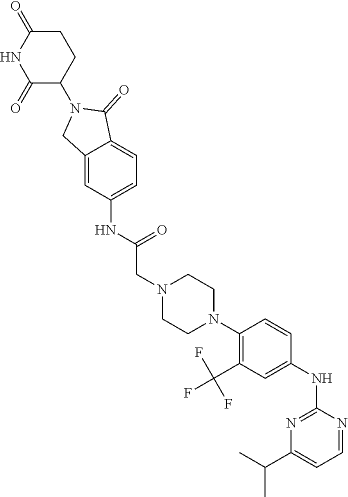 [SMILES: CC(C)c1ccnc(Nc2ccc(N3CCN(CC(=O)Nc4ccc5c(c4)CN(C4CCC(=O)NC4=O)C5=O)CC3)c(C(F)(F)F)c2)n1] | 665.4 |
| 1-132 | 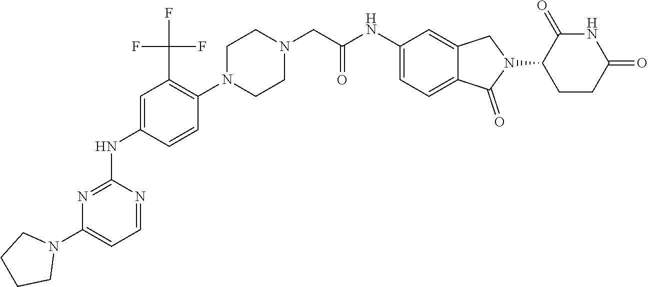 [SMILES: O=C1CC[C@H](N2Cc3cc(NC(=O)CN4CCN(c5ccc(Nc6nccc(N7CCCC7)n6)cc5C(F)(F)F)CC4)ccc3C2=O)C(=O)N1] | 692.4 |
| 1-133 | 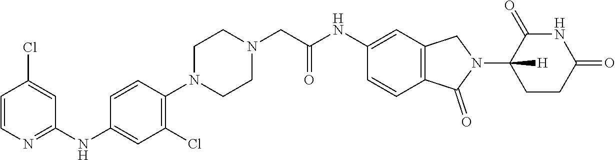 | 622.3 |
| 1-134 | 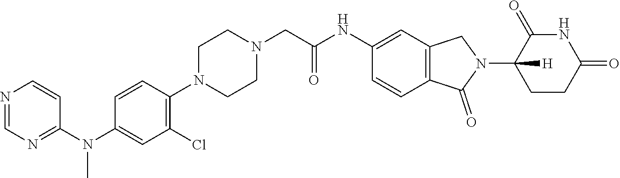 | 603.3 |
| 1-135 | 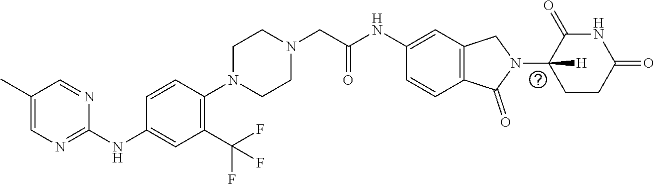 | 665.4 |
| 1-136 | 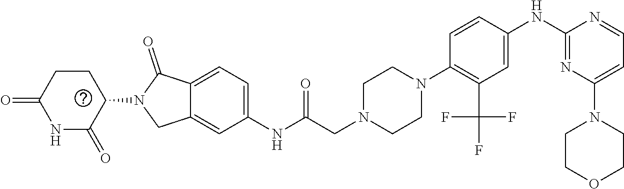 [SMILES: O=C1CC[C@H](N2Cc3cc(NC(=O)CN4CCN(c5ccc(Nc6nccc(N7CCOCC7)n6)cc5C(F)(F)F)CC4)ccc3C2=O)C(=O)N1] | 708.4 |
| 1-137 | 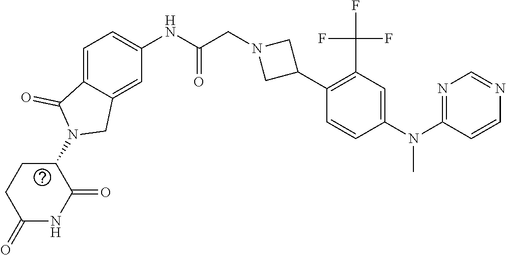 [SMILES: CN(c1ccc(C2CN(CC(=O)Nc3ccc4c(c3)CN([C@H]3CCC(=O)NC3=O)C4=O)C2)c(C(F)(F)F)c1)c1ccncn1] | 608.4 |
| 1-138 | 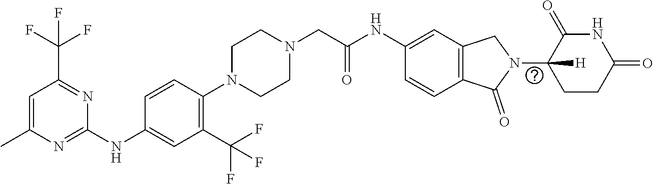 | 705.5 |
| 1-139 | 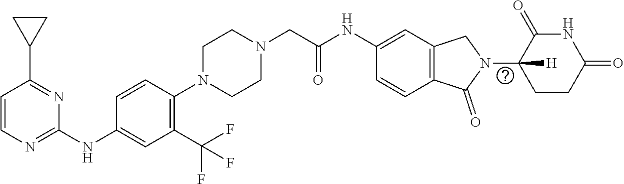 | 663.5 |
| 1-140 | 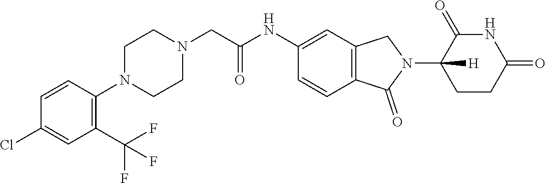 | 563.3 |
| 1-141 | 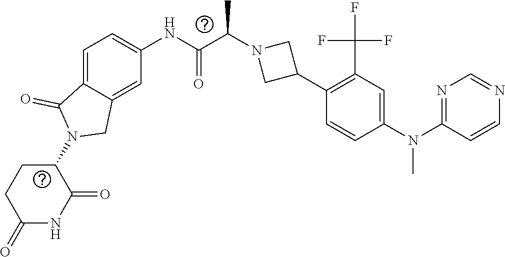 [SMILES: C[C@H](C(=O)Nc1ccc2c(c1)CN([C@H]1CCC(=O)NC1=O)C2=O)N1CC(c2ccc(N(C)c3ccncn3)cc2C(F)(F)F)C1] | 564.4 |
| 1-142 | 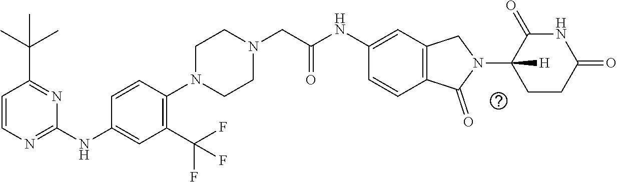 | 622.4 |
| 1-143 | 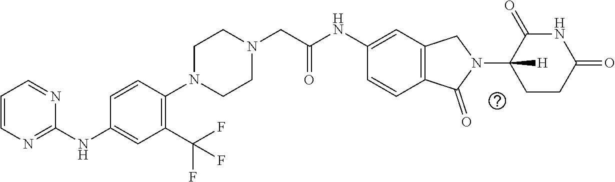 | 679.6 |
| 1-144 | 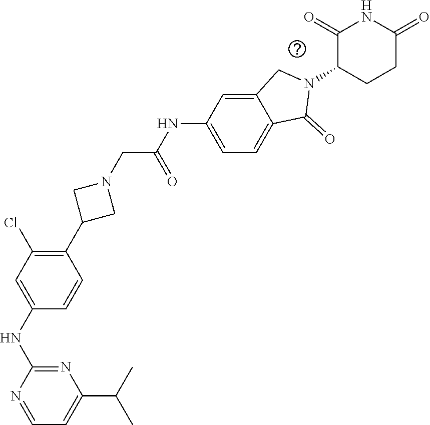 [SMILES: CC(C)c1ccnc(Nc2ccc(C3CN(CC(=O)Nc4ccc5c(c4)CN([C@H]4CCC(=O)NC4=O)C5=O)C3)c(Cl)c2)n1] | 637.5 |
| 1-145 | 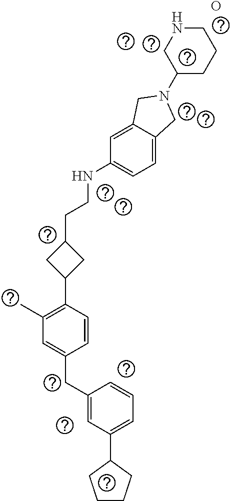 | 602.3 |
| 1-146 | 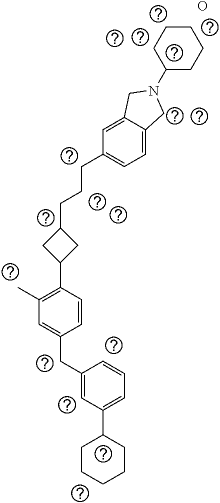 | 629.4 |
| 1-147 | 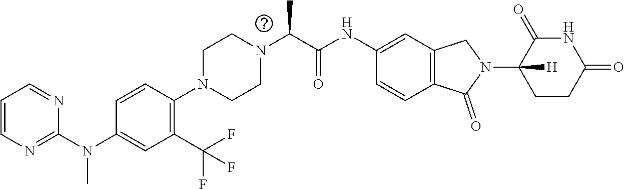 | 651.5 |
| 1-148 | 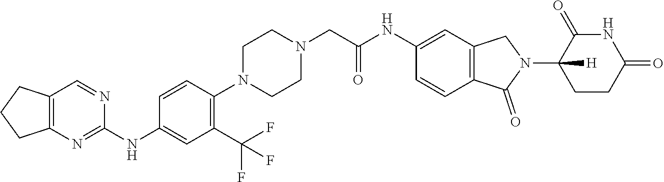 | 663.5 |
| 1-149 | 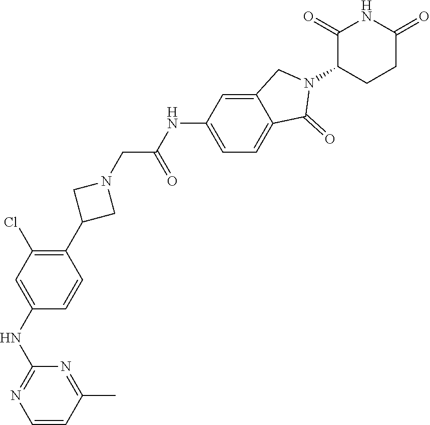 [SMILES: Cc1ccnc(Nc2ccc(C3CN(CC(=O)Nc4ccc5c(c4)CN([C@H]4CCC(=O)NC4=O)C5=O)C3)c(Cl)c2)n1] | 574.4 |
| 1-150 | 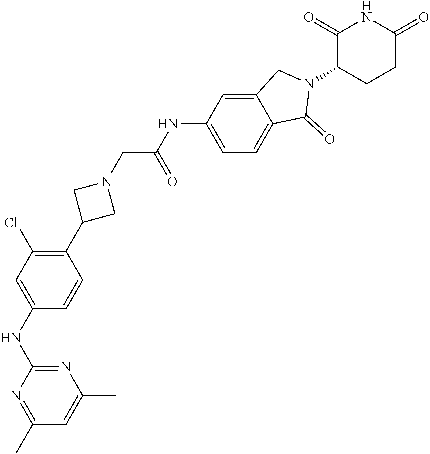 [SMILES: Cc1cc(C)nc(Nc2ccc(C3CN(CC(=O)Nc4ccc5c(c4)CN([C@H]4CCC(=O)NC4=O)C5=O)C3)c(Cl)c2)n1] | 588.4 |
| 1-151 | 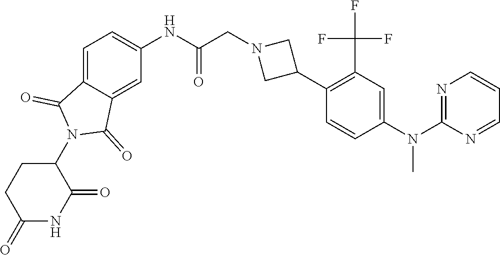 [SMILES: CN(c1ccc(C2CN(CC(=O)Nc3ccc4c(c3)C(=O)N(C3CCC(=O)NC3=O)C4=O)C2)c(C(F)(F)F)c1)c1ncccn1] | 622.4 |
| 1-152 | 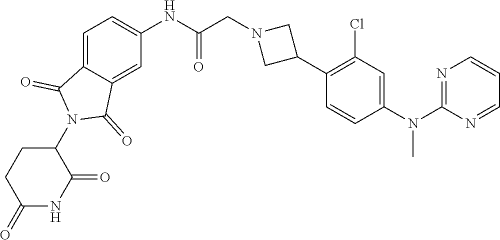 [SMILES: CN(c1ccc(C2CN(CC(=O)Nc3ccc4c(c3)C(=O)N(C3CCC(=O)NC3=O)C4=O)C2)c(Cl)c1)c1ncccn1] | 588.4 |
| 1-153 | 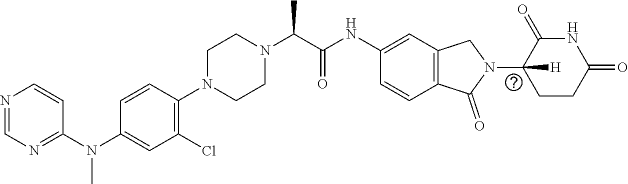 | 617.5 |
| 1-154 | 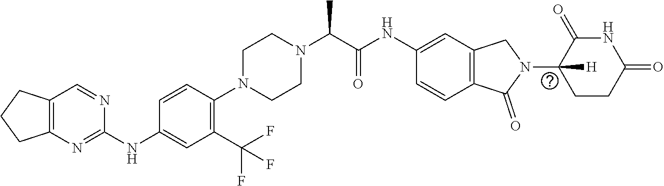 | 677.5 |
| 1-155 | 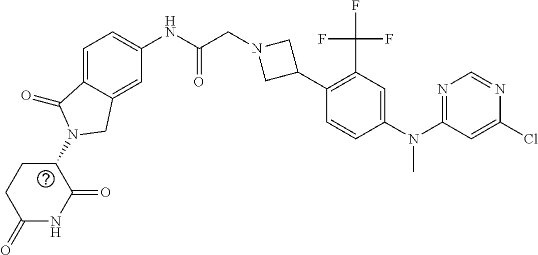 [SMILES: CN(c1ccc(C2CN(CC(=O)Nc3ccc4c(c3)CN([C@H]3CCC(=O)NC3=O)C4=O)C2)c(C(F)(F)F)c1)c1cc(Cl)ncn1] | 642.4 |
| 1-156 | 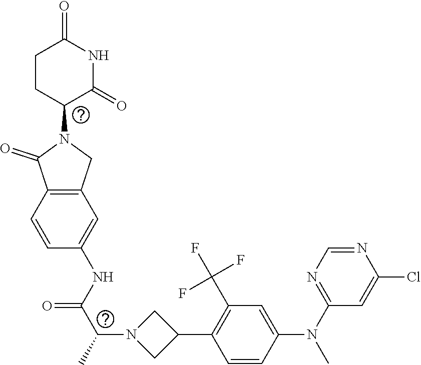 [SMILES: C[C@H](C(=O)Nc1ccc2c(c1)CN([C@H]1CCC(=O)NC1=O)C2=O)N1CC(c2ccc(N(C)c3cc(Cl)ncn3)cc2C(F)(F)F)C1] | 656.5 |
| 1-157 | 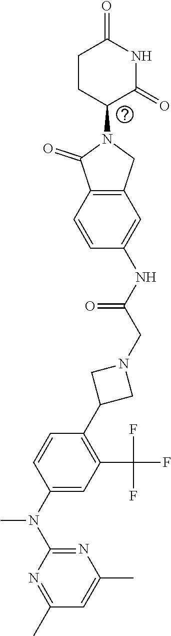 [SMILES: Cc1cc(C)nc(N(C)c2ccc(C3CN(CC(=O)Nc4ccc5c(c4)CN([C@H]4CCC(=O)NC4=O)C5=O)C3)c(C(F)(F)F)c2)n1] | 635.5 |
| 1-158 | 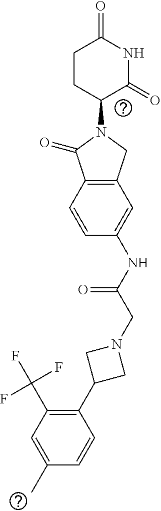 [SMILES: Cc1ccc(C2CN(CC(=O)Nc3ccc4c(c3)CN([C@H]3CCC(=O)NC3=O)C4=O)C2)c(C(F)(F)F)c1] | 535.3 |
| 1-159 | 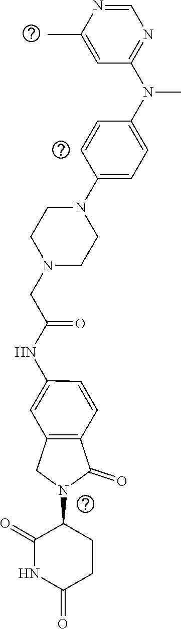 [SMILES: Cc1cc(N(C)c2ccc(N3CCN(CC(=O)Nc4ccc5c(c4)CN([C@H]4CCC(=O)NC4=O)C5=O)CC3)cc2)ncn1] | 637.4 |
| 1-160 | 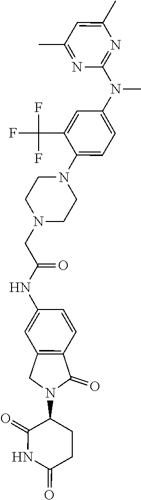 [SMILES: Cc1cc(C)nc(N(C)c2ccc(N3CCN(CC(=O)Nc4ccc5c(c4)CN([C@H]4CCC(=O)NC4=O)C5=O)CC3)c(C(F)(F)F)c2)n1] | 665.4 |
| 1-161 | 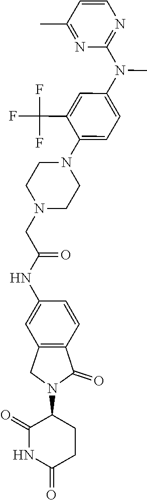 [SMILES: Cc1ccnc(N(C)c2ccc(N3CCN(CC(=O)Nc4ccc5c(c4)CN([C@H]4CCC(=O)NC4=O)C5=O)CC3)c(C(F)(F)F)c2)n1] | 651.5 |
| 1-162 | 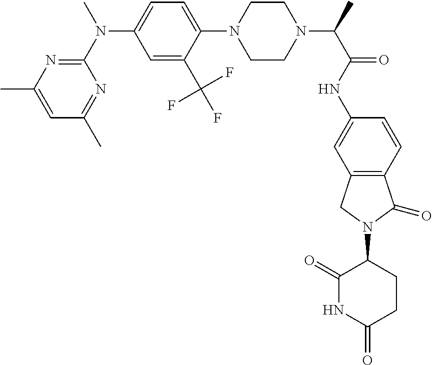 [SMILES: Cc1cc(C)nc(N(C)c2ccc(N3CCN([C@@H](C)C(=O)Nc4ccc5c(c4)CN([C@H]4CCC(=O)NC4=O)C5=O)CC3)c(C(F)(F)F)c2)n1] | 679.5 |
| 1-163 | 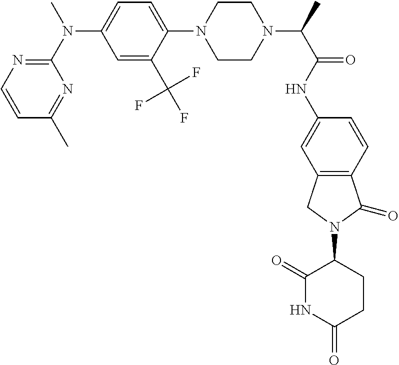 [SMILES: Cc1ccnc(N(C)c2ccc(N3CCN([C@@H](C)C(=O)Nc4ccc5c(c4)CN([C@H]4CCC(=O)NC4=O)C5=O)CC3)c(C(F)(F)F)c2)n1] | 665.3 |
| 1-164 | 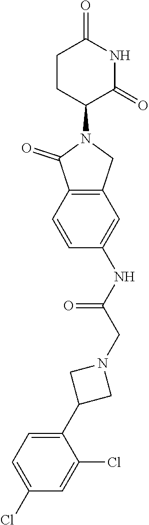 [SMILES: O=C1CC[C@H](N2Cc3cc(NC(=O)CN4CC(c5ccc(Cl)cc5Cl)C4)ccc3C2=O)C(=O)N1] | 501.2 |
| 1-165 | 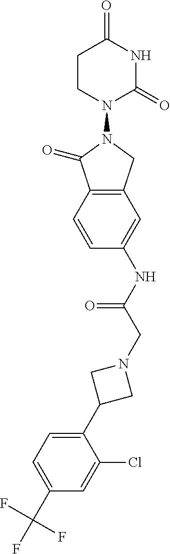 | 535.3 |
| 1-166 | 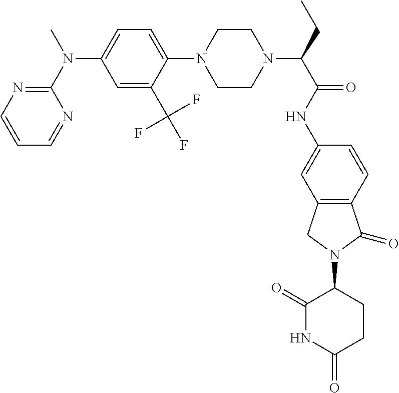 [SMILES: CC[C@@H](C(=O)Nc1ccc2c(c1)CN([C@H]1CCC(=O)NC1=O)C2=O)N1CCN(c2ccc(N(C)c3ncccn3)cc2C(F)(F)F)CC1] | 665.5 |
| 1-167 | 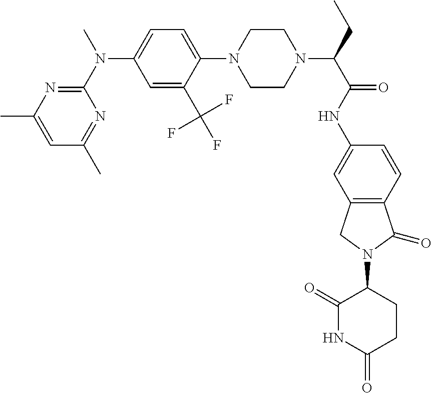 [SMILES: CC[C@@H](C(=O)Nc1ccc2c(c1)CN([C@H]1CCC(=O)NC1=O)C2=O)N1CCN(c2ccc(N(C)c3nc(C)cc(C)n3)cc2C(F)(F)F)CC1] | 693.5 |
| 1-168 | 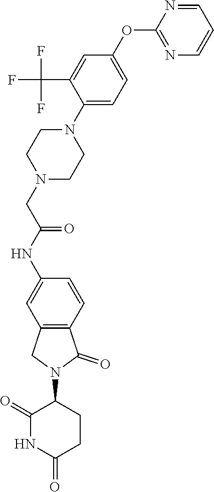 [SMILES: O=C1CC[C@H](N2Cc3cc(NC(=O)CN4CCN(c5ccc(Oc6ncccn6)cc5C(F)(F)F)CC4)ccc3C2=O)C(=O)N1] | 624.5 |
| 1-169 | 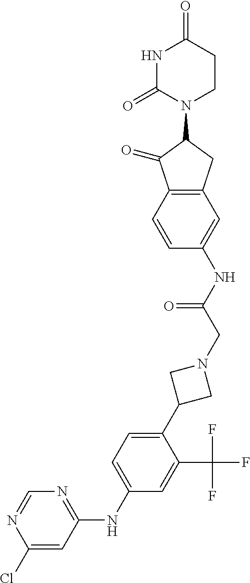 | 628.5 |
| 1-170 | 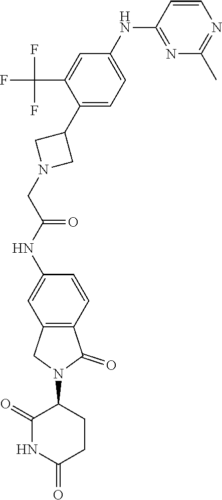 [SMILES: Cc1nccc(Nc2ccc(C3CN(CC(=O)Nc4ccc5c(c4)CN([C@H]4CCC(=O)NC4=O)C5=O)C3)c(C(F)(F)F)c2)n1] | 608.4 |
| 1-171 | 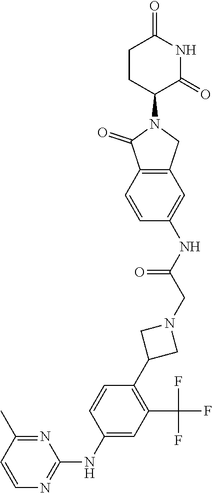 [SMILES: Cc1ccnc(Nc2ccc(C3CN(CC(=O)Nc4ccc5c(c4)CN([C@H]4CCC(=O)NC4=O)C5=O)C3)c(C(F)(F)F)c2)n1] | 608.4 |
| 1-172 | 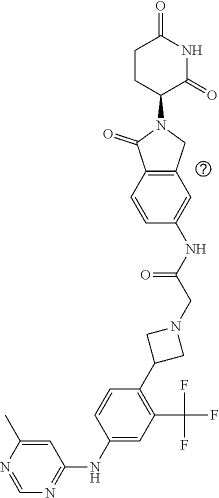 [SMILES: Cc1cc(Nc2ccc(C3CN(CC(=O)Nc4ccc5c(c4)CN([C@H]4CCC(=O)NC4=O)C5=O)C3)c(C(F)(F)F)c2)ncn1] | 608.5 |
| 1-173 | 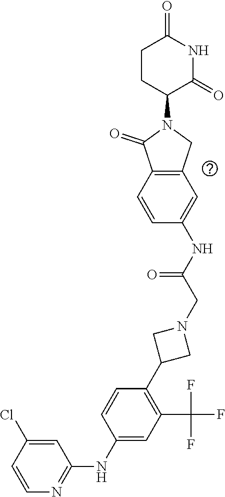 [SMILES: O=C1CC[C@H](N2Cc3cc(NC(=O)CN4CC(c5ccc(Nc6cc(Cl)ccn6)cc5C(F)(F)F)C4)ccc3C2=O)C(=O)N1] | 627.4 |
| 1-174 | 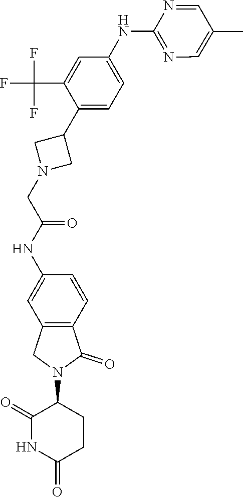 [SMILES: Cc1cnc(Nc2ccc(C3CN(CC(=O)Nc4ccc5c(c4)CN([C@H]4CCC(=O)NC4=O)C5=O)C3)c(C(F)(F)F)c2)nc1] | 608.5 |
| 1-175 | 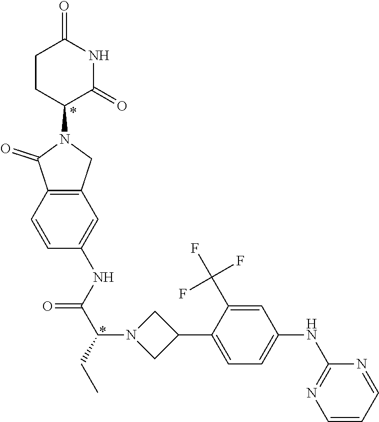 [SMILES: CC[C@H](C(=O)Nc1ccc2c(c1)CN([C@H]1CCC(=O)NC1=O)C2=O)N1CC(c2ccc(Nc3ncccn3)cc2C(F)(F)F)C1] | 622.4 |
| 1-176 | 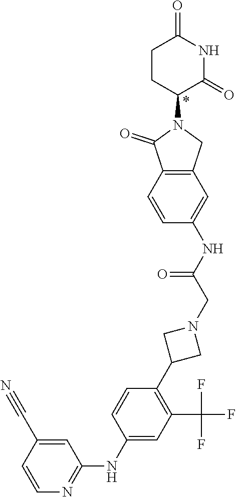 [SMILES: N#Cc1ccnc(Nc2ccc(C3CN(CC(=O)Nc4ccc5c(c4)CN([C@H]4CCC(=O)NC4=O)C5=O)C3)c(C(F)(F)F)c2)c1] | 618.4 |
| 1-177 | 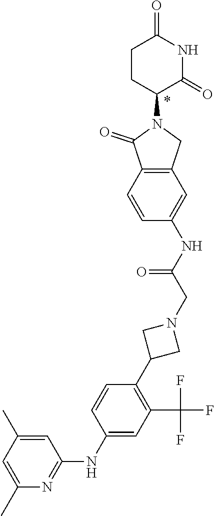 [SMILES: Cc1cc(C)nc(Nc2ccc(C3CN(CC(=O)Nc4ccc5c(c4)CN([C@H]4CCC(=O)NC4=O)C5=O)C3)c(C(F)(F)F)c2)c1] | 621.5 |
| 1-178 | 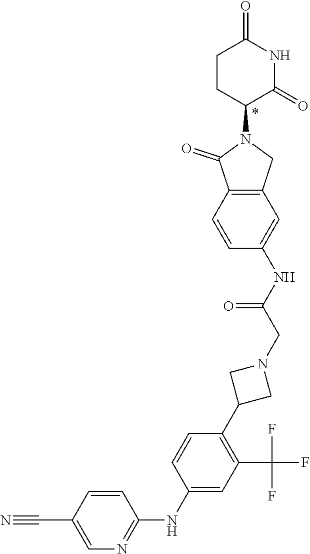 [SMILES: N#Cc1ccc(Nc2ccc(C3CN(CC(=O)Nc4ccc5c(c4)CN([C@H]4CCC(=O)NC4=O)C5=O)C3)c(C(F)(F)F)c2)nc1] | 618.4 |
| 1-179 | 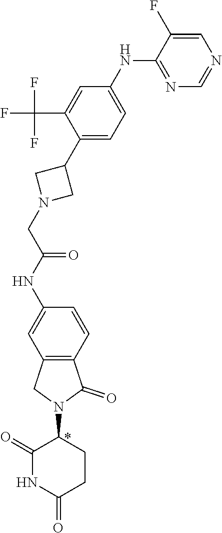 [SMILES: O=C1CC[C@H](N2Cc3cc(NC(=O)CN4CC(c5ccc(Nc6ncncc6F)cc5C(F)(F)F)C4)ccc3C2=O)C(=O)N1] | 612.4 |
| 1-180 | 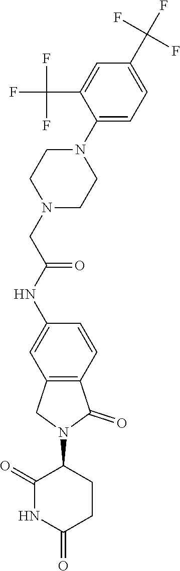 [SMILES: O=C1CC[C@H](N2Cc3cc(NC(=O)CN4CCN(c5ccc(C(F)(F)F)cc5C(F)(F)F)CC4)ccc3C2=O)C(=O)N1] | 598.4 |
| 1-181 | 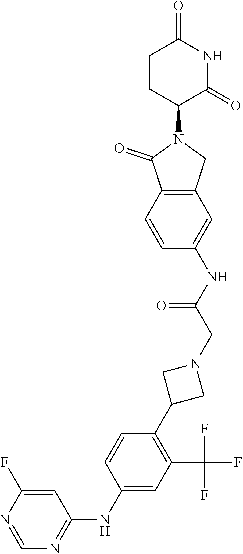 [SMILES: O=C1CC[C@H](N2Cc3cc(NC(=O)CN4CC(c5ccc(Nc6cc(F)ncn6)cc5C(F)(F)F)C4)ccc3C2=O)C(=O)N1] | 612.4 |
| 1-182 | 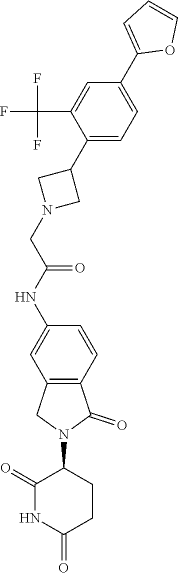 [SMILES: O=C1CC[C@H](N2Cc3cc(NC(=O)CN4CC(c5ccc(-c6ccco6)cc5C(F)(F)F)C4)ccc3C2=O)C(=O)N1] | 567.4 |
| 1-183 | 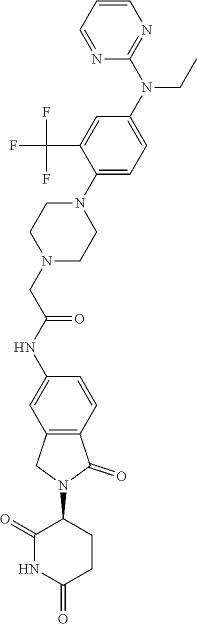 [SMILES: CCN(c1ccc(N2CCN(CC(=O)Nc3ccc4c(c3)CN([C@H]3CCC(=O)NC3=O)C4=O)CC2)c(C(F)(F)F)c1)c1ncccn1] | 651.5 |
| 1-184 | 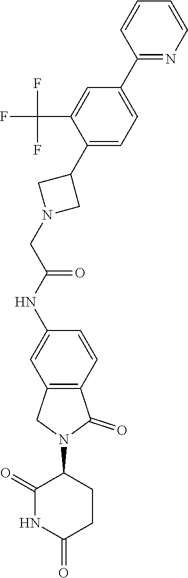 [SMILES: O=C1CC[C@H](N2Cc3cc(NC(=O)CN4CC(c5ccc(-c6ccccn6)cc5C(F)(F)F)C4)ccc3C2=O)C(=O)N1] | 578.4 |
| 1-185 | 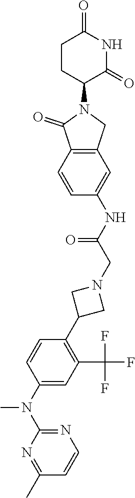 [SMILES: Cc1ccnc(N(C)c2ccc(C3CN(CC(=O)Nc4ccc5c(c4)CN([C@H]4CCC(=O)NC4=O)C5=O)C3)c(C(F)(F)F)c2)n1] | 622.5 |
| 1-186 | 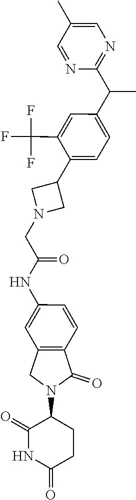 [SMILES: Cc1cnc(C(C)c2ccc(C3CN(CC(=O)Nc4ccc5c(c4)CN([C@H]4CCC(=O)NC4=O)C5=O)C3)c(C(F)(F)F)c2)nc1] | 622.5 |
| 1-187 | 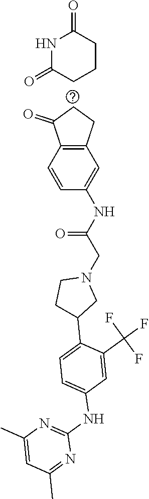 | 636.4 |
| 1-188 | 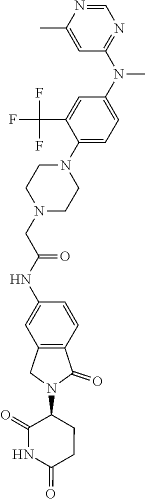 [SMILES: Cc1cc(N(C)c2ccc(N3CCN(CC(=O)Nc4ccc5c(c4)CN([C@H]4CCC(=O)NC4=O)C5=O)CC3)c(C(F)(F)F)c2)ncn1] | 651.5 |
| 1-189 | 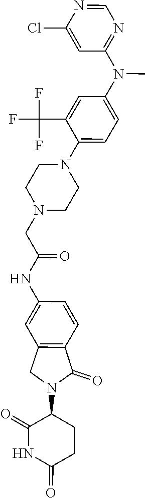 [SMILES: CN(c1ccc(N2CCN(CC(=O)Nc3ccc4c(c3)CN([C@H]3CCC(=O)NC3=O)C4=O)CC2)c(C(F)(F)F)c1)c1cc(Cl)ncn1] | 671.4 |
| 1-190 | 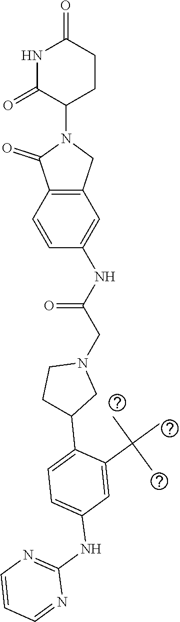 [SMILES: CC(C)(C)c1cc(Nc2ncccn2)ccc1C1CCN(CC(=O)Nc2ccc3c(c2)CN(C2CCC(=O)NC2=O)C3=O)C1] | 608.4 |
| 1-191 | 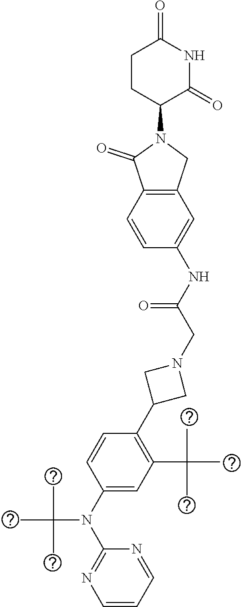 [SMILES: CC(C)(C)c1cc(N(c2ncccn2)C(C)(C)C)ccc1C1CN(CC(=O)Nc2ccc3c(c2)CN([C@H]2CCC(=O)NC2=O)C3=O)C1] | 611.5 |
| 1-192 | 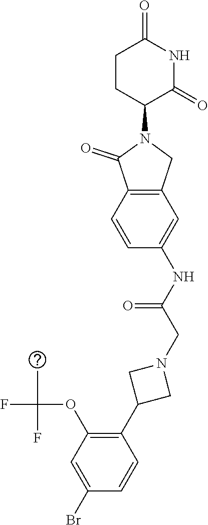 [SMILES: CC(F)(F)Oc1cc(Br)ccc1C1CN(CC(=O)Nc2ccc3c(c2)CN([C@H]2CCC(=O)NC2=O)C3=O)C1] | 595.2 597.2 |
| 1-193 | 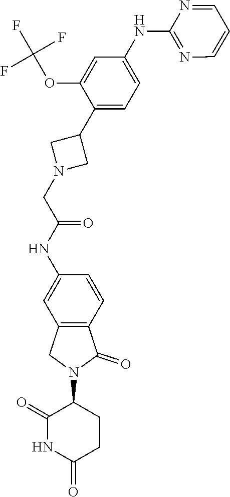 [SMILES: O=C1CC[C@H](N2Cc3cc(NC(=O)CN4CC(c5ccc(Nc6ncccn6)cc5OC(F)(F)F)C4)ccc3C2=O)C(=O)N1] | 610.4 |
| 1-194 | 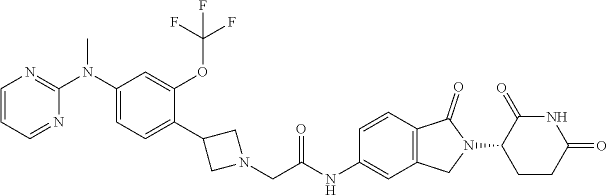 [SMILES: CN(c1ccc(C2CN(CC(=O)Nc3ccc4c(c3)CN([C@H]3CCC(=O)NC3=O)C4=O)C2)c(OC(F)(F)F)c1)c1ncccn1] | 624.4 |
| 1-195 | 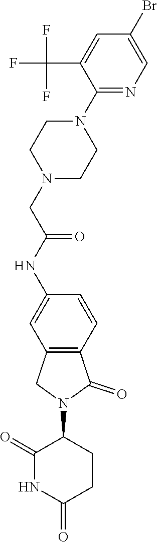 [SMILES: O=C1CC[C@H](N2Cc3cc(NC(=O)CN4CCN(c5ncc(Br)cc5C(F)(F)F)CC4)ccc3C2=O)C(=O)N1] | 609.3 611.3 |
| 1-196 | 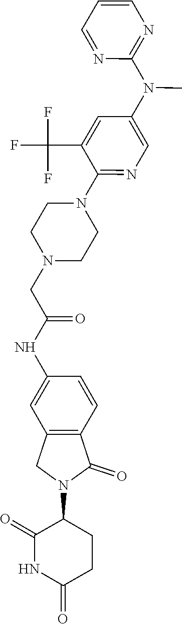 [SMILES: CN(c1cnc(N2CCN(CC(=O)Nc3ccc4c(c3)CN([C@H]3CCC(=O)NC3=O)C4=O)CC2)c(C(F)(F)F)c1)c1ncccn1] | 638.5 |
| 1-197 | 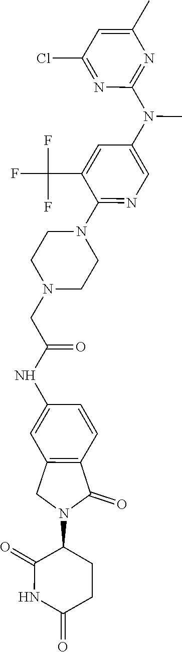 [SMILES: Cc1cc(Cl)nc(N(C)c2cnc(N3CCN(CC(=O)Nc4ccc5c(c4)CN([C@H]4CCC(=O)NC4=O)C5=O)CC3)c(C(F)(F)F)c2)n1] | 686.5 |
| 1-198 | 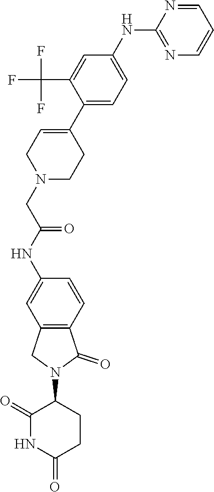 [SMILES: O=C1CC[C@H](N2Cc3cc(NC(=O)CN4CC=C(c5ccc(Nc6ncccn6)cc5C(F)(F)F)CC4)ccc3C2=O)C(=O)N1] | 620.5 |
| 1-199 | 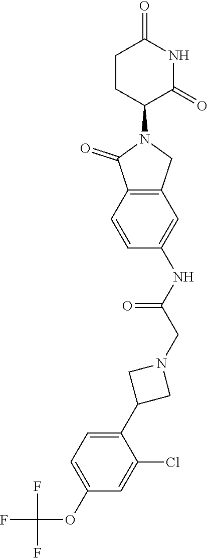 [SMILES: O=C1CC[C@H](N2Cc3cc(NC(=O)CN4CC(c5ccc(OC(F)(F)F)cc5Cl)C4)ccc3C2=O)C(=O)N1] | 551.3 |
| 1-200 | 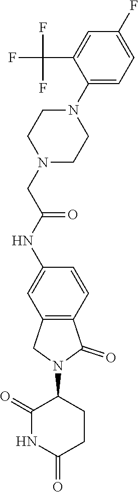 [SMILES: O=C1CC[C@H](N2Cc3cc(NC(=O)CN4CCN(c5ccc(F)cc5C(F)(F)F)CC4)ccc3C2=O)C(=O)N1] | 519.3 |
| 1-201 | 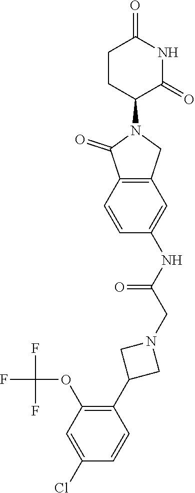 [SMILES: O=C1CC[C@H](N2Cc3cc(NC(=O)CN4CC(c5ccc(Cl)cc5OC(F)(F)F)C4)ccc3C2=O)C(=O)N1] | 551.3 |
| 1-202 | 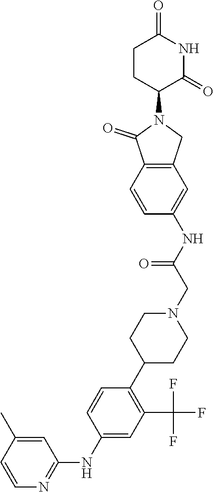 [SMILES: Cc1ccnc(Nc2ccc(C3CCN(CC(=O)Nc4ccc5c(c4)CN([C@H]4CCC(=O)NC4=O)C5=O)CC3)c(C(F)(F)F)c2)c1] | 636.4 |
| 1-203 | 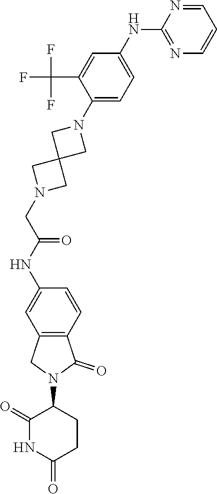 [SMILES: O=C1CC[C@H](N2Cc3cc(NC(=O)CN4CC5(C4)CN(c4ccc(Nc6ncccn6)cc4C(F)(F)F)C5)ccc3C2=O)C(=O)N1] | 635.5 |
| 1-204 | 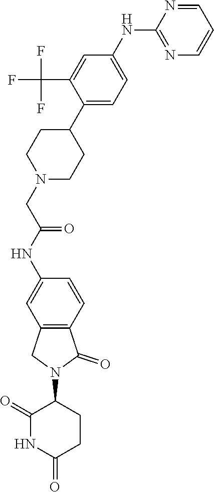 [SMILES: O=C1CC[C@H](N2Cc3cc(NC(=O)CN4CCC(c5ccc(Nc6ncccn6)cc5C(F)(F)F)CC4)ccc3C2=O)C(=O)N1] | 622.4 |
| 1-205 | 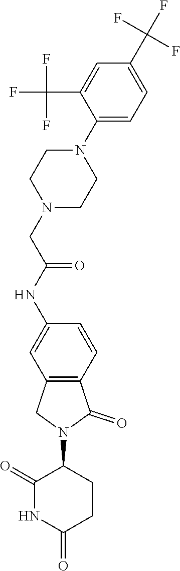 [SMILES: O=C1CC[C@H](N2Cc3cc(NC(=O)CN4CCN(c5ccc(C(F)(F)F)cc5C(F)(F)F)CC4)ccc3C2=O)C(=O)N1] | 599.4 |
| 1-206 | 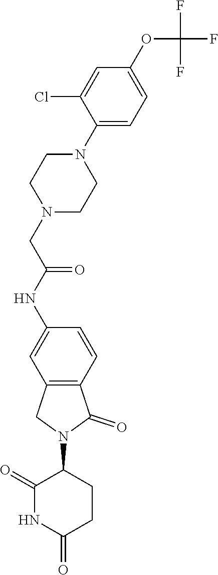 [SMILES: O=C1CC[C@H](N2Cc3cc(NC(=O)CN4CCN(c5ccc(OC(F)(F)F)cc5Cl)CC4)ccc3C2=O)C(=O)N1] | 580.4 |
| 1-207 | 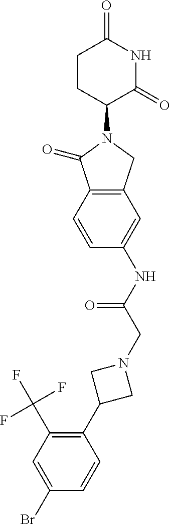 [SMILES: O=C1CC[C@H](N2Cc3cc(NC(=O)CN4CC(c5ccc(Br)cc5C(F)(F)F)C4)ccc3C2=O)C(=O)N1] | 579.3 581.3 |
| 1-208 | 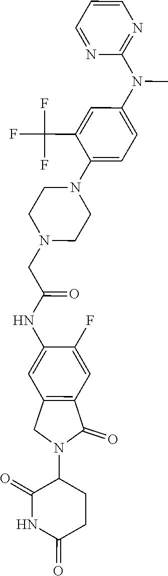 [SMILES: CN(c1ccc(N2CCN(CC(=O)Nc3cc4c(cc3F)C(=O)N(C3CCC(=O)NC3=O)C4)CC2)c(C(F)(F)F)c1)c1ncccn1] | 655.3 |
| 1-209 | 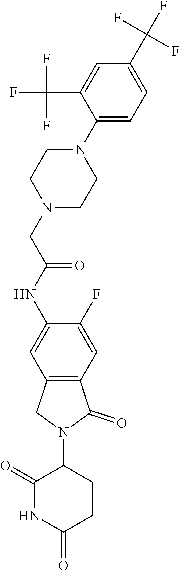 [SMILES: O=C1CCC(N2Cc3cc(NC(=O)CN4CCN(c5ccc(C(F)(F)F)cc5C(F)(F)F)CC4)c(F)cc3C2=O)C(=O)N1] | 616.3 |
| 1-210 | 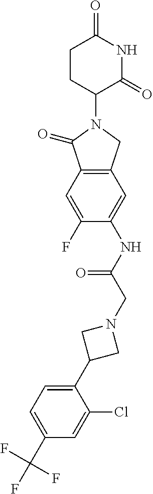 [SMILES: O=C1CCC(N2Cc3cc(NC(=O)CN4CC(c5ccc(C(F)(F)F)cc5Cl)C4)c(F)cc3C2=O)C(=O)N1] | 553.1 |
| 1-211 | 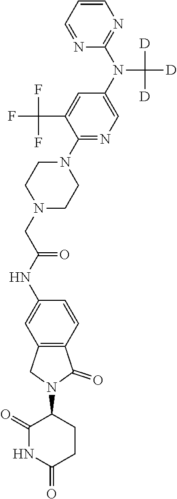 [SMILES: [2H]C([2H])([2H])N(c1cnc(N2CCN(CC(=O)Nc3ccc4c(c3)CN([C@H]3CCC(=O)NC3=O)C4=O)CC2)c(C(F)(F)F)c1)c1ncccn1] | 641.3 |
| 1-212 | 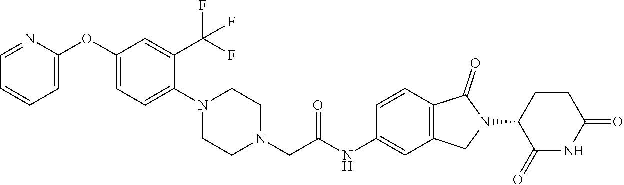 [SMILES: O=C1CC[C@@H](N2Cc3cc(NC(=O)CN4CCN(c5ccc(Oc6ccccn6)cc5C(F)(F)F)CC4)ccc3C2=O)C(=O)N1] | 623.3 |
| 1-213 | 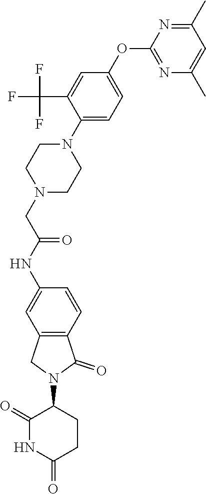 [SMILES: Cc1cc(C)nc(Oc2ccc(N3CCN(CC(=O)Nc4ccc5c(c4)CN([C@H]4CCC(=O)NC4=O)C5=O)CC3)c(C(F)(F)F)c2)n1] | 652.4 |
| 1-214 | 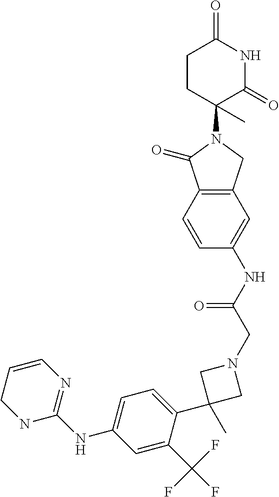 [SMILES: CC1(c2ccc(NC3=NC=CCN3)cc2C(F)(F)F)CN(CC(=O)Nc2ccc3c(c2)CN([C@@]2(C)CCC(=O)NC2=O)C3=O)C1] | 608.3 |
| 1-215 | 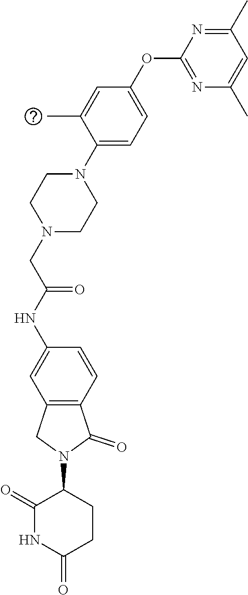 [SMILES: Cc1cc(C)nc(Oc2ccc(N3CCN(CC(=O)Nc4ccc5c(c4)CN([C@H]4CCC(=O)NC4=O)C5=O)CC3)c(C)c2)n1] | 618.3 |
| 1-216 | 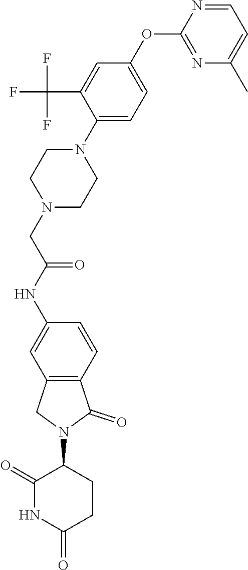 [SMILES: Cc1ccnc(Oc2ccc(N3CCN(CC(=O)Nc4ccc5c(c4)CN([C@H]4CCC(=O)NC4=O)C5=O)CC3)c(C(F)(F)F)c2)n1] | 638.4 |
| 1-217 | 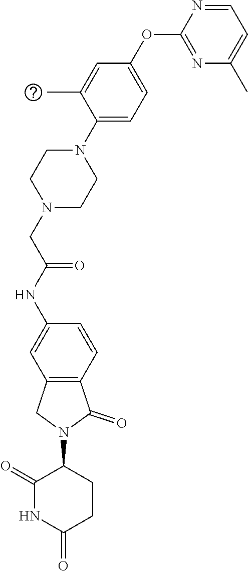 [SMILES: Cc1ccnc(Oc2ccc(N3CCN(CC(=O)Nc4ccc5c(c4)CN([C@H]4CCC(=O)NC4=O)C5=O)CC3)c(C)c2)n1] | 604.4 |
| 1-218 | 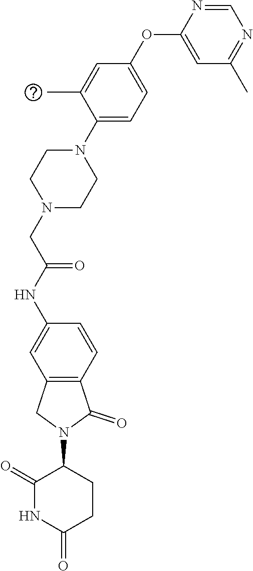 [SMILES: Cc1cc(Oc2ccc(N3CCN(CC(=O)Nc4ccc5c(c4)CN([C@H]4CCC(=O)NC4=O)C5=O)CC3)c(C)c2)ncn1] | 604.4 |
| 1-219 | 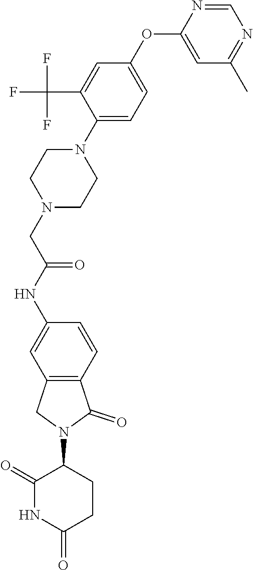 [SMILES: Cc1cc(Oc2ccc(N3CCN(CC(=O)Nc4ccc5c(c4)CN([C@H]4CCC(=O)NC4=O)C5=O)CC3)c(C(F)(F)F)c2)ncn1] | 638.5 |
| 1-220 | 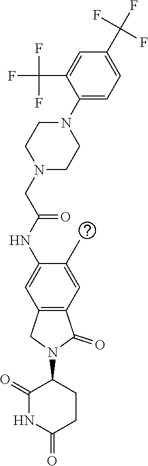 [SMILES: Cc1cc2c(cc1NC(=O)CN1CCN(c3ccc(C(F)(F)F)cc3C(F)(F)F)CC1)CN([C@H]1CCC(=O)NC1=O)C2=O] | 616.1 |
| | ¹H NMR (400 MHz, DMSO-d6) δ ppm 1.97-2.02 (m, | |
| | 1H), 2.33 (d, J = 1.6 Hz, 2H), 2.37-2.45 (m, 2H), 2.59 | |
| | (d, J = 18.4 Hz, 2H), 2.67 (s, 4H), 2.84 (s, 2H), 2.92 (s, | |
| | 4H), 4.23-4.35 (m, 3H), 4.40 (d, J = 17.5 Hz, 1H), | |
| | 5.08 (d, J = 13.5 Hz, 1H), 6.52 (s, 1H), 6.71-6.79 (m, | |
| | 2H), 7.10 (d, J = 9.4 Hz, 1H), 7.43-7.58 (m, 4H), 7.96 | |
| | (s, 1H), 8.14 (s, 1H), 9.05 (s, 1H), 10.98 (s, 1H) | |
| 1-221 | 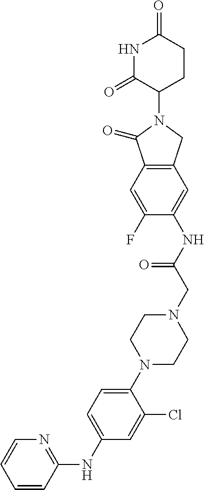 [SMILES: O=C1CCC(N2Cc3cc(NC(=O)CN4CCN(c5ccc(Nc6ccccn6)cc5Cl)CC4)c(F)cc3C2=O)C(=O)N1] | 607.0 |
| 1-222 | 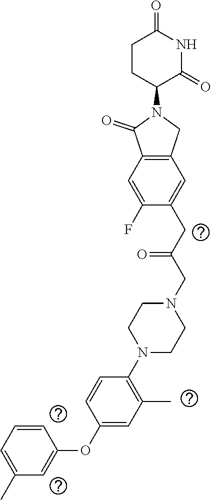 [SMILES: Cc1cccc(Oc2ccc(N3CCN(CC(=O)Cc4cc5c(cc4F)C(=O)N([C@H]4CCC(=O)NC4=O)C5)CC3)c(C)c2)c1] | 622.0 |
| | ¹H NMR (400 MHz, DMSO-d6) δ ppm 1.98-2.05 (m, | |
| | 1H), 2.31-2.39 (m, 1H), 2.41 (br s, 3H), 2.55-2.69 | |
| | (m, 1H), 2.78 (br s, 4H), 2.85-2.99 (m, 1H), 3.07 (br s, | |
| | 4H), 3.34 (br s, 2H), 4.32 (d, J = 17.6 Hz, 1H), 4.46 (d, | |
| | J = 17.2 Hz, 1H), 5.10 (dd, J = 13.2, 5.2 Hz, 1H), 7.15 | |
| | (d, J = 5.2 Hz, 2H), 7.25 (d, J = 8.8 Hz, 1H), 7.33 (d, J = | |
| | 2.4 Hz, 1H), 7.65 (d, J = 9.6 Hz, 1H), 8.40-8.46 (m, | |
| | 1H), 9.96 (br s, 1H), 11.00 (br s, 1H) | |
| 1-223 | 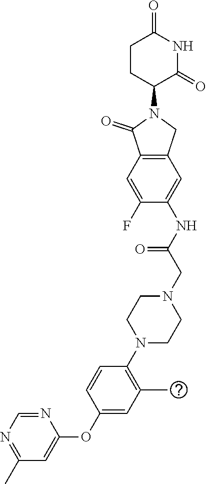 [SMILES: Cc1cc(Oc2ccc(N3CCN(CC(=O)Nc4cc5c(cc4F)C(=O)N([C@H]4CCC(=O)NC4=O)C5)CC3)c(C)c2)ncn1] | 622.0 |
| | ¹H NMR (400 MHz, DMSO-d6) δ ppm 1.96-2.04 (m, | |
| | 1H), 2.31-2.38 (m, 1H), 2.44 (br s, 3H), 2.54-2.70 | |
| | (m, 1H), 2.78 (br s, 4H), 2.86-2.96 (m, 1H), 3.07 (br s, | |
| | 4H), 3.34 (br s, 2H), 4.32 (d, J = 17.6 Hz, 1H), 4.46 (d, | |
| | J = 17.2 Hz, 1H), 5.10 (dd, J = 13.2, 4.8 Hz, 1H), 7.02 | |
| | (br s, 1H), 7.17 (dd, J = 8.8, 2.4 Hz, 1H), 7.27 (d, J = | |
| | 8.8 Hz, 1H), 7.36 (d, J = 2.4 Hz, 1H), 7.65 (d, J = 9.6 | |
| | Hz, 1H), 8.40 (d, J = 6.8 Hz, 1H), 8.63 (br s, 1H), 9.96 | |
| | (br s, 1H), 11.00 (br s, 1H) | |
| 1-224 | 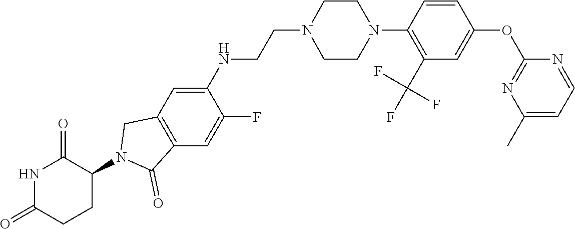 [SMILES: Cc1ccnc(Oc2ccc(N3CCN(CCNc4cc5c(cc4F)C(=O)N([C@H]4CCC(=O)NC4=O)C5)CC3)c(C(F)(F)F)c2)n1] | 656.1 |
| | ¹H NMR (400 MHz, DMSO-d6) δ ppm 1.96-2.05 (m, | |
| | 1 H), 2.30-2.39 (m, 1 H), 2.42 (br s, 3H), 2.55-2.80 | |
| | (m, 5 H), 2.84-3.03 (m, 5 H), 3.34 (br s, 2 H), 4.32 (d, | |
| | J = 17.2 Hz, 1H), 4.46 (d, J = 17.2 Hz, 1 H), 5.10 (dd, | |
| | J = 13.2, 4.8 Hz, 1 H), 7.18 (d, J = 5.2 Hz, 1 H), 7.51- | |
| | 7.53 (m, 2 H), 7.64-7.68 (m, 2 H), 8.41 (d, J = 6.4 Hz, | |
| | 1 H), 8.47 (d, J = 5.2 Hz, 1 H), 9.96 (br s, 1 H), 11.00 | |
| | (br s, 1 H) | |
| 1-225 | 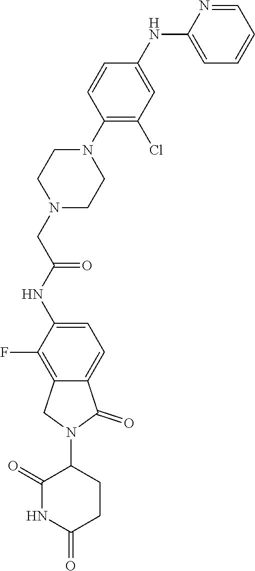 [SMILES: O=C1CCC(N2Cc3c(ccc(NC(=O)CN4CCN(c5ccc(Nc6ccccn6)cc5Cl)CC4)c3F)C2=O)C(=O)N1] | 606.2 |
| 1-226 | 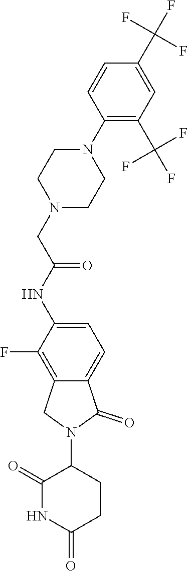 [SMILES: O=C1CCC(N2Cc3c(ccc(NC(=O)CN4CCN(c5ccc(C(F)(F)F)cc5C(F)(F)F)CC4)c3F)C2=O)C(=O)N1] | 636.0 |
| 1-227 | 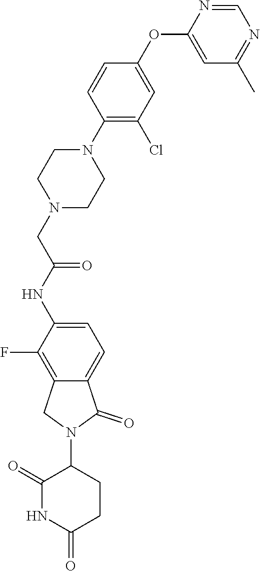 [SMILES: Cc1cc(Oc2ccc(N3CCN(CC(=O)Nc4ccc5c(c4F)CN(C4CCC(=O)NC4=O)C5=O)CC3)c(Cl)c2)ncn1] | 622.2 |
| 1-228 | 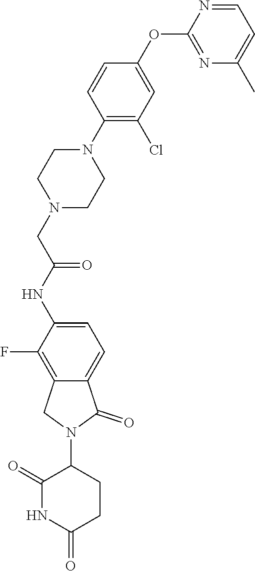 [SMILES: Cc1ccnc(Oc2ccc(N3CCN(CC(=O)Nc4ccc5c(c4F)CN(C4CCC(=O)NC4=O)C5=O)CC3)c(Cl)c2)n1] | 622.0 |
| 1-229 | 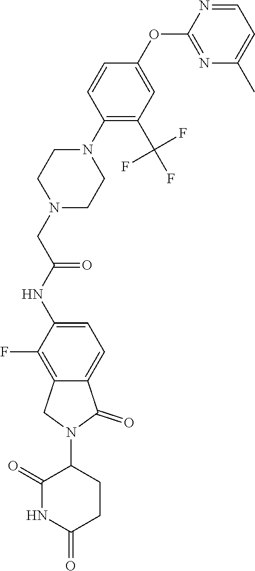 [SMILES: Cc1ccnc(Oc2ccc(N3CCN(CC(=O)Nc4ccc5c(c4F)CN(C4CCC(=O)NC4=O)C5=O)CC3)c(C(F)(F)F)c2)n1] | 656.2 |
| 1-230 | 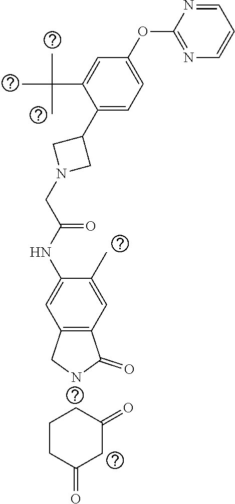 | 613.0 |
| | ¹H NMR (400 MHz, CD3OD) δ ppm 2.14-2.24 (m, | |
| | 1H), 2.44-2.59 (m, 1H), 2.73-2.83 (m, 1H), 2.86- | |
| | 2.93 (m, 1H), 4.44-4.74 (m, 9H), 5.16 (dd, J = 13.4, | |
| | 5.2 Hz, 1H), 7.27 (t, J = 4.8 Hz, 1H), 7.60-7.66 (m, | |
| | 3H), 7.86 (d, J = 8.2 Hz, 1H), 8.29-8.33 (m, 1H), 8.62 | |
| | (d, J = 4.8 Hz, 2H) | |
| 1-231 | 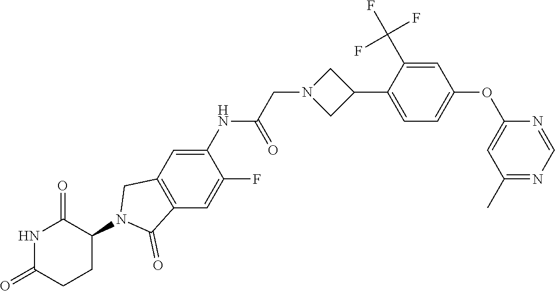 [SMILES: Cc1cc(Oc2ccc(C3CN(CC(=O)Nc4cc5c(cc4F)C(=O)N([C@H]4CCC(=O)NC4=O)C5)C3)c(C(F)(F)F)c2)ncn1] | 627.2 |
| | ¹H NMR (400 MHz, CD3OD) δ ppm 2.13-2.23 (m, | |
| | 1H), 2.46-2.58 (m, 4H), 2.74-2.81 (m, 1H), 2.85- | |
| | 2.92 (m, 1H), 3.74-3.83 (m, 4H), 4.14 (t, J = 8.2 Hz, | |
| | 2H), 4.24-4.39 (m, 1H), 4.59 (q, J = 17.2 Hz, 2H), | |
| | 5.15 (dd, J = 132, 5.2 Hz, 1H), 7.04 (br s, 1H), 7.50- | |
| | 7.55 (m, 2H), 7.63 (d, J = 8.0 Hz, 1H), 7.96 (d, J = 9.2 | |
| | Hz, 1H), 8.25-8.35 (m, 1H), 8.58 (br s, 1H) | |
| 1-232 | 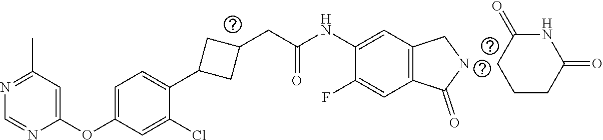 | 593.0 |
| | ¹H NMR (400 MHz, DMSO-d6) δ ppm 1.97-2.08 (m, | |
| | 1H), 2.33-2.40 (m, 1H), 2.45 (br s, 3H), 2.56-2.67 | |
| | (m, 1H), 2.85-2.96 (m, 1H), 3.32-3.42 (m, 4H), 3.90- | |
| | 3.98 (m, 3H), 4.31 (d, J = 17.4 Hz, 1H), 4.44 (d, J = | |
| | 17.4 Hz, 1H), 5.10 (dd, J = 13.2, 5.0 Hz, 1H), 7.06 (s, | |
| | 1H), 7.23 (dd, J = 8.4, 2.4 Hz, 1H), 7.38 (d, J = 2.8 Hz, | |
| | 1H), 7.61 (dd, J = 9.0, 3.2 Hz, 2H), 8.32 (d, J = 6.4 Hz, | |
| | 1H), 8.63 (br s, 1H), 9.82 (br s, 1H), 11.01 (br s, 1H) | |
| 1-233 | 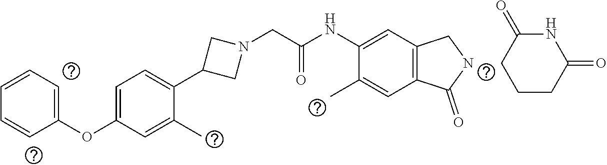 | 579.2 |
| | ¹H NMR (400 MHz, DMSO-d6) δ ppm 1.99-2.02 (m, | |
| | 1H), 2.32-2.43 (m, 1H), 2.57-2.69 (m, 1H), 2.86- | |
| | 2.96 (m, 1H), 3.32-3.43 (m, 4H), 3.90-3.98 (m, 3H), | |
| | 4.31 (d, J = 17.4 Hz, 1H), 4.44 (d, J = 17.2 Hz, 1H), | |
| | 5.10 (dd, J = 13.2, 5.2 Hz, 1H), 7.24 (dd, J = 8.4, 2.2 | |
| | Hz, 1H), 7.30 (t, J = 4.8 Hz, 1H), 7.39 (d, J = 2.2 Hz, | |
| | 1H), 7.61 (dd, J = 12.4, 9.6 Hz, 2H), 8.33 (d, J = 6.6 | |
| | Hz, 1H), 8.66 (d, J = 4.8 Hz, 2H), 9.82 (br s, 1H), | |
| | 10.99 (br s, 1H) | |
| 1-234 | 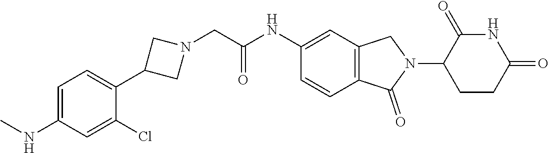 [SMILES: CNc1ccc(C2CN(CC(=O)Nc3ccc4c(c3)CN(C3CCC(=O)NC3=O)C4=O)C2)c(Cl)c1] | 496.1 |
| | ¹H NMR (400 MHz, DMSO-d6) δ ppm 1.94-2.04 (m, | |
| | 1H), 2.33-2.41 (m, 1H), 2.53-2.59 (m, 1H), 2.62 (s, | |
| | 3H), 2.67 (s, 4H), 2.90 (s, 4H), 3.24 (s, 2H), 4.30 (d, J = | |
| | 17.2 Hz, 1H), 4.44 (d, J = 17.2 Hz, 1H), 5.09 (dd, J = | |
| | 13.6, 5.0 Hz, 1H), 5.62 (s, 1H), 6.47 (dd, J = 8.8, 2.6 | |
| | Hz, 1H), 6.57 (d, J = 2.6 Hz, 1H), 7.00 (d, J = 8.8 Hz, | |
| | 1H), 7.72-7.65 (m, 2H), 8.02 (s, 1H), 8.16 (s, 1H), | |
| | 10.09 (s, 1H), 10.98 (s, 1H) | |
| 1-235 | 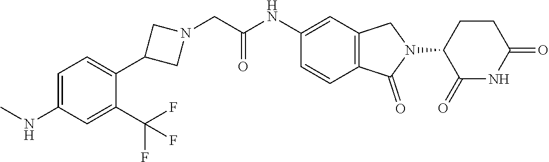 [SMILES: CNc1ccc(C2CN(CC(=O)Nc3ccc4c(c3)CN([C@@H]3CCC(=O)NC3=O)C4=O)C2)c(C(F)(F)F)c1] | 530.3 |
| | ¹H NMR (400 MHz, DMSO-d6) δ ppm 1.94-2.09 (m, | |
| | 1 H), 2.27-2.45 (m, 1 H), 2.60 (d, J = 16.2 Hz, 1 H), | |
| | 2.69 (br s, 3 H), 2.85-3.04 (m, 3 H), 3.25 (d, J = 39.6 | |
| | Hz, 4 H), 3.60 (br s, 2 H), 4.33 (t, J = 8.4 Hz, 3 H), | |
| | 4.47 (d, J = 17.6 Hz, 1 H), 5.10 (dd, J = 13.2, 5.2 Hz, 1 | |
| | H), 6.73-6.87 (m, 2 H), 7.33 (d, J = 8.6 Hz, 1 H), 7.70 | |
| | (dd, J = 32.0, 8.2 Hz, 2 H), 7.94 (br s, 1 H), 10.19 (br s, | |
| | 1 H) | |
| 1-236 | 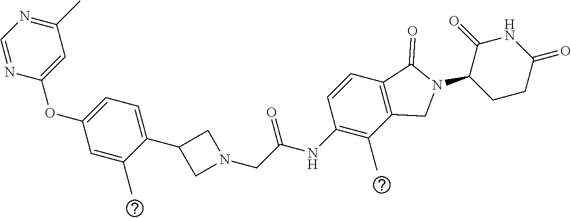 [SMILES: Cc1cc(Oc2ccc(C3CN(CC(=O)Nc4ccc5c(c4C)CN([C@@H]4CCC(=O)NC4=O)C5=O)C3)c(C)c2)ncn1] | 593.1 |
| | ¹H NMR (400 MHz, CD3OD) δ ppm 2.15-2.20 (m, | |
| | 1H), 2.45-2.56 (m, 4H), 2.75-2.81 (m, 1H), 2.85- | |
| | 2.95 (m, 1H), 4.26-4.80 (m, 9H), 5.15 (dd, J = 13.2, | |
| | 5.2 Hz, 1H), 7.02 (br s, 1H), 7.26 (d, J = 8.4 Hz, 1H), | |
| | 7.39 (d, J = 2.0 Hz, 1H), 7.52 (br s, 1H), 7.64 (d, J = | |
| | 8.4 Hz, 1H), 8.28-8.33 (m, 1H), 8.56 (br s, 1H) | |
| 1-237 | 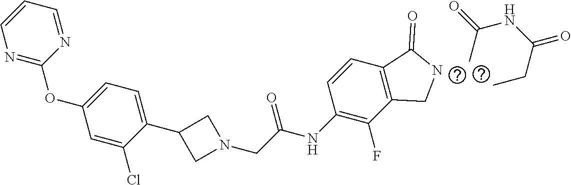 | 579.2 |
| | ¹H NMR (400 MHz, DMSO-d6) δ ppm 1.99-2.02 (m, | |
| | 1H), 2.32-2.40 (m, 1H), 2.57-2.67 (m, 1H), 2.86- | |
| | 2.66 (m, 1H), 3.54 (br s, 4H), 4.02 (br s, 3H), 4.41 (d, J = | |
| | 17.2 Hz, 1H), 4.58 (d, J = 17.2 Hz, 1H), 5.11 (dd, J = | |
| | 13.2, 5.0 Hz, 1H), 7.25 (dd, J = 8.8, 2.4 Hz, 1H), 7.30 | |
| | (t, J = 4.8 Hz, 1H), 7.40 (d, J = 2.4 Hz, 1H), 7.57-7.61 | |
| | (m, 2H), 8.13 (t, J = 7.4 Hz, 1H), 8.66 (d, J = 4.8 Hz, | |
| | 2H), 9.97 (br s, 1H), 11.01 (br s, 1H) | |
| 1-238 | 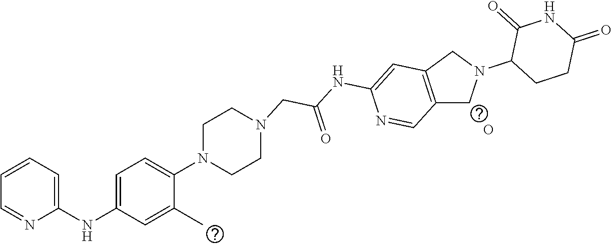 | 589.2 |
| | ¹H NMR (400 MHz, DMSO-d6) δ ppm 1.98-2.03 (m, | |
| | 1H), 2.32-2.44 (m, 1H), 2.54-2.75 (m, 5H), 2.85- | |
| | 3.00 (m, 5H), 3.33 (br s, 2H), 4.42 (d, J = 18.8 Hz, 1H), | |
| | 4.56 (d, J = 18.8 Hz, 1H), 5.11 (dd, J = 13.2, 5.0 Hz, | |
| | 1H), 6.70-6.80 (m, 2H), 7.15 (d, J = 8.8 Hz, 1H), 7.47 | |
| | (dd, J = 8.8, 2.4 Hz, 1H), 7.52-7.60 (m, 1H), 7.97 (d, J = | |
| | 2.8 Hz, 1H), 8.15 (dd, J = 5.2, 1.6 Hz, 1H), 8.36 (br | |
| | s, 1H), 8.71 (br s, 1H), 9.06 (br s, 1H), 10.37 (br s, 1H), | |
| | 11.01 (br s, 1H) | |
| 1-239 | 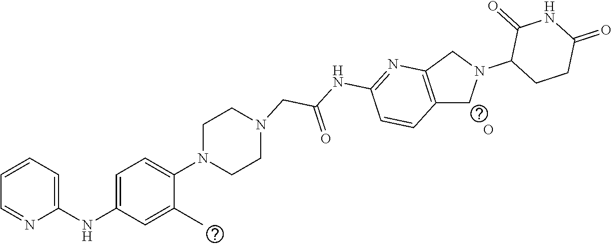 | 589.2 |
| | ¹H NMR (400 MHz, DMSO-d6) δ ppm 1.98-2.07 (m, | |
| | 1H), 2.35-2.44 (m, 1H), 2.56-2.66 (m, 1H), 2.74 (s, | |
| | 4H), 2.85-2.93 (m, 1H), 2.98 (s, 4H), 3.34 (s, 2H), 4.38 | |
| | (dd, J = 65.4, 17.8 Hz, 2H), 5.16 (dd, J = 13.2, 5.0 Hz, | |
| | 1H), 6.63-6.83 (m, 2H), 7.14 (d, J = 8.8 Hz, 1H), 7.42- | |
| | 7.63 (m, 2H), 7.96 (d, J = 2.2 Hz, 1H), 8.16-8.23 (m, | |
| | 2H), 8.30 (d, J = 8.6 Hz, 1H), 9.05 (s, 1H), 10.43 (s, | |
| | 1H), 11.00 (s, 1H) | |
| 1-240 | 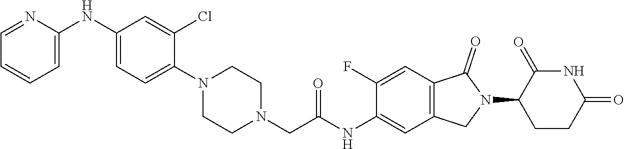 [SMILES: O=C1CC[C@@H](N2Cc3cc(NC(=O)CN4CCN(c5ccc(Nc6ccccn6)cc5Cl)CC4)c(F)cc3C2=O)C(=O)N1] | 607.0 |
| 1-241 | 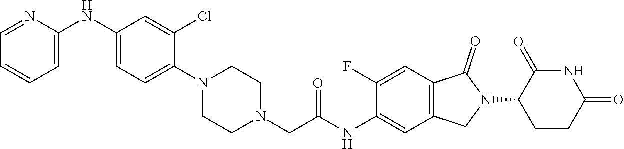 [SMILES: O=C1CC[C@H](N2Cc3cc(NC(=O)CN4CCN(c5ccc(Nc6ccccn6)cc5Cl)CC4)c(F)cc3C2=O)C(=O)N1] | 607.0 |
| 1-242 | 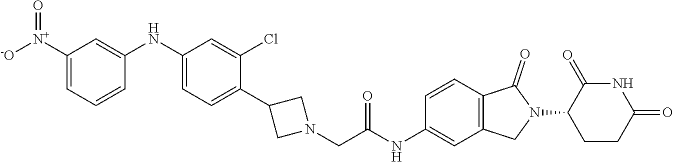 | 603.5 |
| 1-243 | 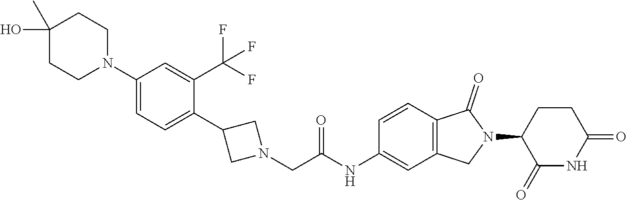 [SMILES: CC1(O)CCN(c2ccc(C3CN(CC(=O)Nc4ccc5c(c4)CN([C@H]4CCC(=O)NC4=O)C5=O)C3)c(C(F)(F)F)c2)CC1] | 600.3, 300.7 |
| 1-244 | 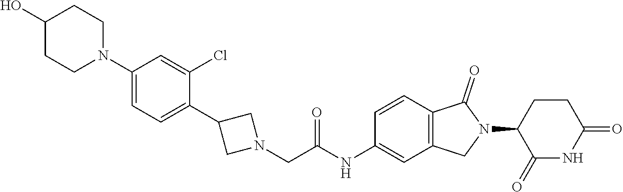 [SMILES: O=C1CC[C@H](N2Cc3cc(NC(=O)CN4CC(c5ccc(N6CCC(O)CC6)cc5Cl)C4)ccc3C2=O)C(=O)N1] | 566.4, 283.9 |
| 1-245 | 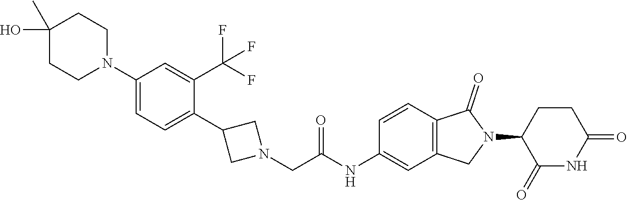 [SMILES: CC1(O)CCN(c2ccc(C3CN(CC(=O)Nc4ccc5c(c4)CN([C@H]4CCC(=O)NC4=O)C5=O)C3)c(C(F)(F)F)c2)CC1] | 307.8, 614.3 |
| 1-246 | 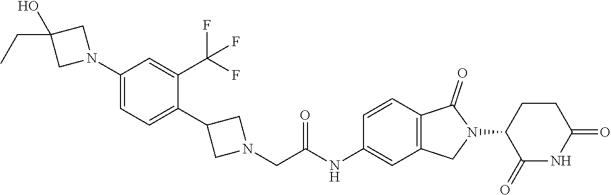 [SMILES: CCC1(O)CN(c2ccc(C3CN(CC(=O)Nc4ccc5c(c4)CN([C@@H]4CCC(=O)NC4=O)C5=O)C3)c(C(F)(F)F)c2)C1] | 660.4, 300.7 |
| 1-247 | 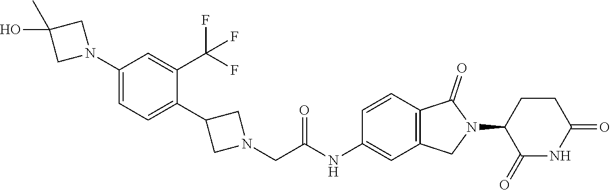 [SMILES: CC1(O)CN(c2ccc(C3CN(CC(=O)Nc4ccc5c(c4)CN([C@H]4CCC(=O)NC4=O)C5=O)C3)c(C(F)(F)F)c2)C1] | 293.7, 586.1 |
| 1-248 | 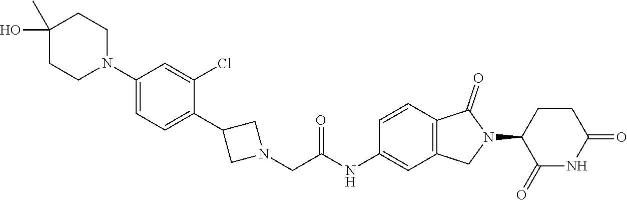 [SMILES: CC1(O)CCN(c2ccc(C3CN(CC(=O)Nc4ccc5c(c4)CN([C@H]4CCC(=O)NC4=O)C5=O)C3)c(Cl)c2)CC1] | 580.4, 290.7 |
| 1-249 | 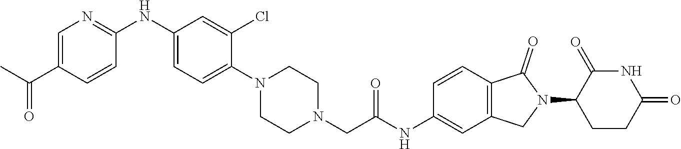 [SMILES: CC(=O)c1ccc(Nc2ccc(N3CCN(CC(=O)Nc4ccc5c(c4)CN([C@@H]4CCC(=O)NC4=O)C5=O)CC3)c(Cl)c2)nc1] | 630.2, 315.7 |
| 1-250 | 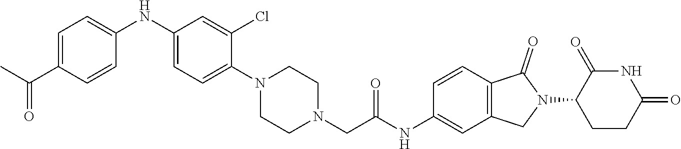 [SMILES: CC(=O)c1ccc(Nc2ccc(N3CCN(CC(=O)Nc4ccc5c(c4)CN([C@H]4CCC(=O)NC4=O)C5=O)CC3)c(Cl)c2)cc1] | 629.2 |
| 1-251 | 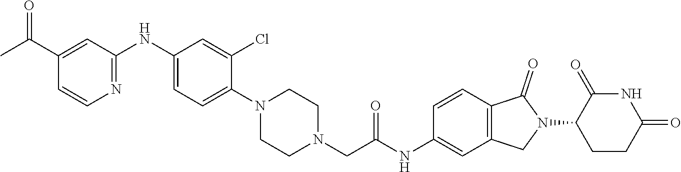 [SMILES: CC(=O)c1ccnc(Nc2ccc(N3CCN(CC(=O)Nc4ccc5c(c4)CN([C@H]4CCC(=O)NC4=O)C5=O)CC3)c(Cl)c2)c1] | 630.4, 315.6 |
| 1-252 | 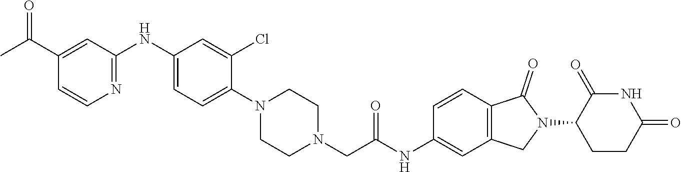 [SMILES: CC(=O)c1ccnc(Nc2ccc(N3CCN(CC(=O)Nc4ccc5c(c4)CN([C@H]4CCC(=O)NC4=O)C5=O)CC3)c(Cl)c2)c1] | 629.3, 315.6 |
| 1-253 | 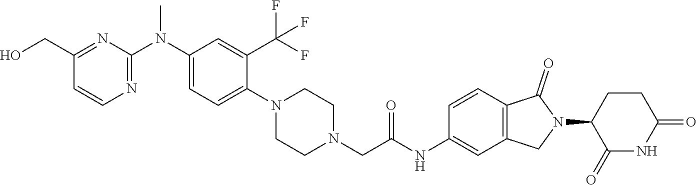 [SMILES: CN(c1ccc(N2CCN(CC(=O)Nc3ccc4c(c3)CN([C@H]3CCC(=O)NC3=O)C4=O)CC2)c(C(F)(F)F)c1)c1nccc(CO)n1] | 667.1 |
| | 1H NMR (400 MHz, DMSO-d6) δ ppm 1.98-2.05 (m, | |
| | 1H), 2.32-2.40 (m, 1H), 2.54 (s, 3H), 2.56-2.70 (m, | |
| | 5H), 2.84-3.00 (m, 5H), 3.47 (s, 2H), 4.28-4.37 (m, | |
| | 3H), 4.44 (d, J = 17.4 Hz, 1H), 5.06-5.15 (m, 2H), | |
| | 7.59-7.72 (m, 5H), 8.04 (s, 1H), 8.34 (s, 2H), 10.13 (s, | |
| | 1H), 10.98 (s, 1H) | |
| 1-254 | 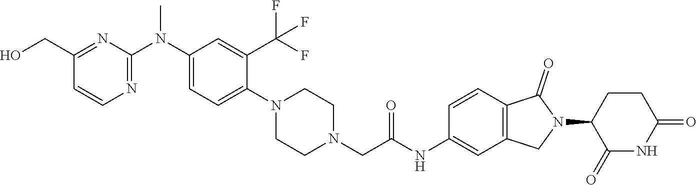 [SMILES: CN(c1ccc(N2CCN(CC(=O)Nc3ccc4c(c3)CN([C@H]3CCC(=O)NC3=O)C4=O)CC2)c(C(F)(F)F)c1)c1nccc(CO)n1] | 667.2 |
| | ¹H NMR (400 MHz, CD3OD) δ 2.12-2.22 (m, 1H), | |
| | 2.41-2.56 (m, 1H), 2.74-2.81 (m, 1H), 2.87 (d, J = | |
| | 5.4 Hz, 1H), 2.89-2.99 (m, 4H), 3.11 (d, J = 4.2 Hz, | |
| | 4H), 3.47 (s, 2H), 3.52 (s, 3H), 4.41-4.56 (m, 4H), | |
| | 5.14 (dd, J = 13.2, 5.2 Hz, 1H), 6.90 (d, J = 5.2 Hz, | |
| | 1H) 7.53-7.68 (m, 4H), 7.78 (d, J = 8.4 Hz, 1H), 8.09 | |
| | (s, 1H), 8.29 (d, J = 5.2 Hz, 1H). | |
| 1-255 | 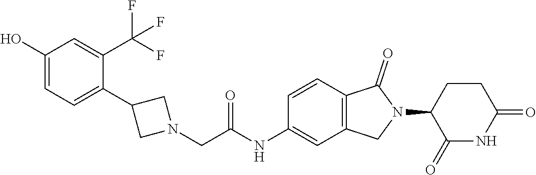 [SMILES: O=C1CC[C@H](N2Cc3cc(NC(=O)CN4CC(c5ccc(O)cc5C(F)(F)F)C4)ccc3C2=O)C(=O)N1] | 517.2 |
| 1-256 | 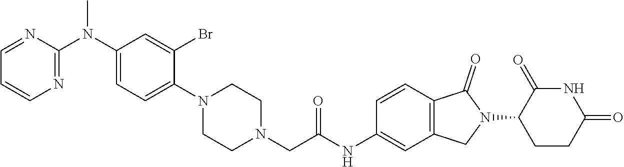 [SMILES: CN(c1ccc(N2CCN(CC(=O)Nc3ccc4c(c3)CN([C@H]3CCC(=O)NC3=O)C4=O)CC2)c(Br)c1)c1ncccn1] | 647.3, 649.3 |
| | 1H NMR (400 MHz, DMSO_d6) δ 1.95-2.03 (m, | |
| | 1H), 2.31-2.42 (m, 2H), 2.55-2.65 (m, 2H), 2.74 (s, | |
| | 4H), 2.86-2.97 (m, 2H), 3.06 (s, 4H), 3.28 (s, 2H), | |
| | 3.42 (s, 3H), 4.30 (d, J = 17.6 Hz, 1H), 4.44 (d, J = 17.6 | |
| | Hz, 1H), 5.09 (dd, J = 13.2, 5.2 Hz, 1H), 6.74 (d, J = 4.8 | |
| | Hz, 1H), 7.21 (d, J = 8.4 Hz, 1H), 7.32 (dd, J = 8.4, 2.4 | |
| | Hz, 1H), 7.57 (d, J = 2.4 Hz, 1H), 7.64-7.72 (m, 2H), | |
| | 8.04 (s, 1H), 8.37 (d, J = 4.8 Hz, 2H), 10.15 (s, 1H), | |
| | 10.98 (s, 1H) | |
| 1-257 | 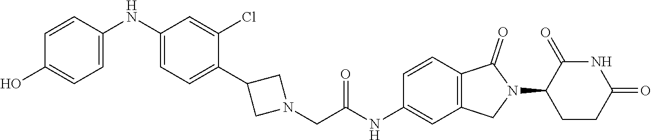 [SMILES: O=C1CC[C@@H](N2Cc3cc(NC(=O)CN4CC(c5ccc(Nc6ccc(O)cc6)cc5Cl)C4)ccc3C2=O)C(=O)N1] | 574.5 |
| 1-258 | 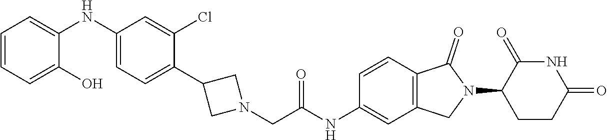 [SMILES: O=C1CC[C@@H](N2Cc3cc(NC(=O)CN4CC(c5ccc(Nc6ccccc6O)cc5Cl)C4)ccc3C2=O)C(=O)N1] | 574.4 |
| 1-259 | 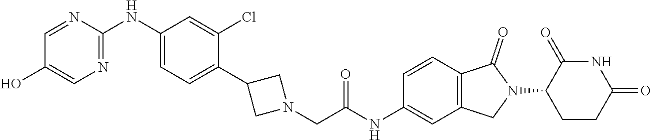 [SMILES: O=C1CC[C@H](N2Cc3cc(NC(=O)CN4CC(c5ccc(Nc6ncc(O)cn6)cc5Cl)C4)ccc3C2=O)C(=O)N1] | 576.4 |
| 1-260 | 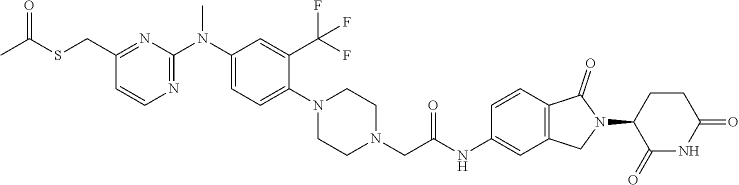 [SMILES: CC(=O)SCc1ccnc(N(C)c2ccc(N3CCN(CC(=O)Nc4ccc5c(c4)CN([C@H]4CCC(=O)NC4=O)C5=O)CC3)c(C(F)(F)F)c2)n1] | 363.2, 725.1 |
| | ¹H NMR (400 MHz, DMSO_d6) δ 1.96-2.06 (m, | |
| | 1H), 2.30-2.44 (m, 4H), 2.55-2.82 (m, 5H), 2.84- | |
| | 3.12 (m, 5H), 3.18-3.30 (m, 2H), 3.47 (s, 3H), 4.04 | |
| | (s, 2H), 4.38 (dd, J = 56.0, 17.4 Hz, 2H), 5.09 (dd, J = | |
| | 13.2, 5.0 Hz, 1H), 6.78 (d, J = 5.2 Hz, 1H), 7.52-7.75 | |
| | (m, 5H), 8.02 (s, 1H), 8.32 (d, J = 5.0 Hz, 1H), 10.14 | |
| | (s, 1H), 10.98 (s, 1H). | |
| 1-261 | 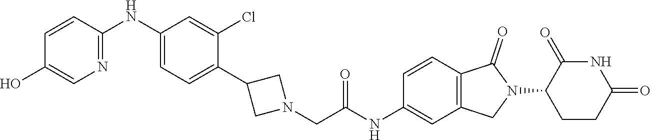 [SMILES: O=C1CC[C@H](N2Cc3cc(NC(=O)CN4CC(c5ccc(Nc6ccc(O)cn6)cc5Cl)C4)ccc3C2=O)C(=O)N1] | 575.4 |
| 1-262 | 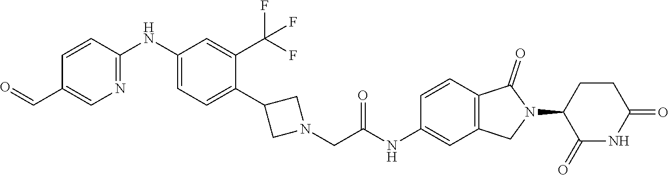 [SMILES: O=Cc1ccc(Nc2ccc(C3CN(CC(=O)Nc4ccc5c(c4)CN([C@H]4CCC(=O)NC4=O)C5=O)C3)c(C(F)(F)F)c2)nc1] | 621.4 |
| 1-263 | 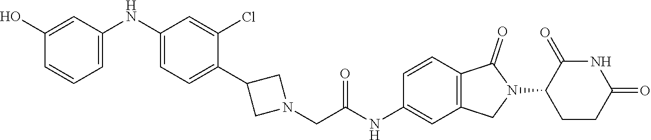 [SMILES: O=C1CC[C@H](N2Cc3cc(NC(=O)CN4CC(c5ccc(Nc6cccc(O)c6)cc5Cl)C4)ccc3C2=O)C(=O)N1] | 574.3 |
| 1-264 | 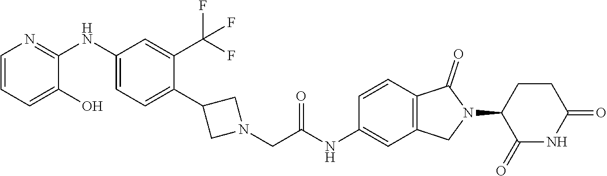 [SMILES: O=C1CC[C@H](N2Cc3cc(NC(=O)CN4CC(c5ccc(Nc6ncccc6O)cc5C(F)(F)F)C4)ccc3C2=O)C(=O)N1] | 609.4, 305.2 |
| 1-265 | 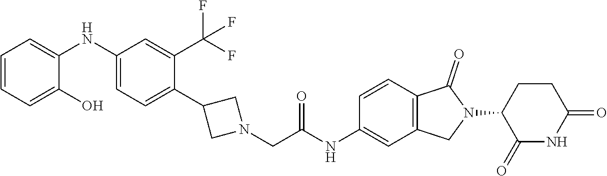 [SMILES: O=C1CC[C@@H](N2Cc3cc(NC(=O)CN4CC(c5ccc(Nc6ccccc6O)cc5C(F)(F)F)C4)ccc3C2=O)C(=O)N1] | 608.3 |
| 1-266 | 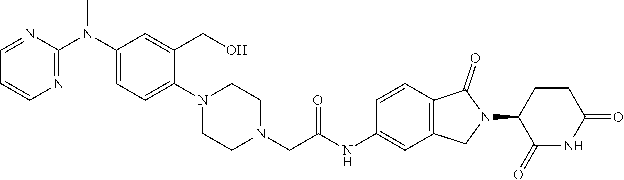 [SMILES: CN(c1ccc(N2CCN(CC(=O)Nc3ccc4c(c3)CN([C@H]3CCC(=O)NC3=O)C4=O)CC2)c(CO)c1)c1ncccn1] | 599.1, 300.2 |
| | ¹H NMR (400 MHz, DMSO_d6) δ 1.96-2.04 (m, | |
| | 1H), 2.36-2.42 (m, 1H), 2.53-2.86 (m, 5H), 2.86- | |
| | 3.08 (m, 5H), 3.41 (s, 3H), 4.31 (d, J = 17.2 Hz, 1H), | |
| | 4.45 (d, J = 17.2 Hz, 1H), 4.56 (s, 2H), 5.16-5.05 (m, | |
| | 2H), 6.70 (t, J = 4.8 Hz, 1H), 7.10 (d, J = 8.4 Hz, 1H), | |
| | 7.13-7.18 (m, 1H), 7.35 (s, 1H), 7.70 (s, 2H), 8.01 (s, | |
| | 1H), 8.34 (d, J = 4.8 Hz, 2H), 10.13 (br s, 1H), 10.99 | |
| | (s, 1H) | |
| 1-267 | 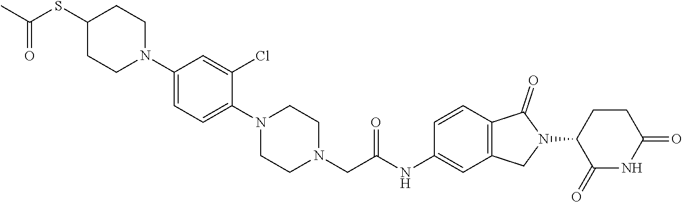 [SMILES: CC(=O)SC1CCN(c2ccc(N3CCN(CC(=O)Nc4ccc5c(c4)CN([C@@H]4CCC(=O)NC4=O)C5=O)CC3)c(Cl)c2)CC1] | 653.3, 327.3 |
| | ¹H NMR (400 MHz, CD3OD) δ 1.70-1.78 (m, 2H), | |
| | 2.00-2.08 (m, 2H), 2.12-2.22 (m, 1H), 2.30 (s, 3H), | |
| | 2.42-2.55 (m, 1H), 2.77-2.95 (m, 8H), 3.03-3.11 | |
| | (m, 4H), 3.34 (s, 2H), 3.38-3.50 (m, 2H), 3.54-3.64 | |
| | (m, 1H), 4.48 (q, J = 17.2 Hz, 2H), 5.13 (dd, J = 13.2, | |
| | 4.8 Hz, 1H), 6.89 (dd, J = 8.8, 2.8 Hz, 1H), 7.00 (d, J = | |
| | 2.8 Hz, 1H), 7.09 (d, J = 8.8 Hz, 1H), 7.63 (d, J = 9.2 | |
| | Hz, 1H), 7.76 (d, J = 8.4 Hz, 1H), 8.07 (s, 1H). | |
| 1-268 | 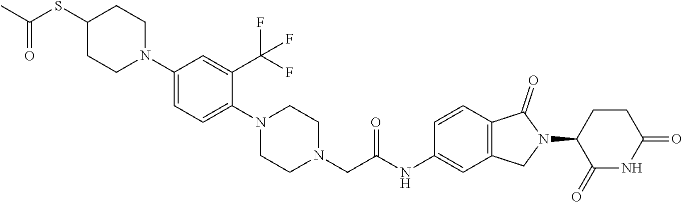 [SMILES: CC(=O)SC1CCN(c2ccc(N3CCN(CC(=O)Nc4ccc5c(c4)CN([C@H]4CCC(=O)NC4=O)C5=O)CC3)c(C(F)(F)F)c2)CC1] | 687.3, 344.3 |
| | ¹H NMR (400 MHz, DMSO_d6) δ 1.58-1.70 (m, | |
| | 2H), 1.91-2.03 (m, 3H), 2.33 (s, 3H), 2.38 (dd, J = | |
| | 13.2, 4.8 Hz, 1H), 2.56-2.71 (m, 5H), 2.86 (s, 4H), | |
| | 2.90-3.03 (m, 3H), 3.24 (s, 2H), 3.47-3.61 (m, 3H), | |
| | 4.30 (d, J = 17.2 Hz, 1H), 4.44 (d, J = 17.2 Hz, 1H), | |
| | 5.09 (dd, J = 13.2, 5.2 Hz, 1H), 7.10 (d, J = 2.8 Hz, | |
| | 1H), 7.17-7.26 (m, 1H), 7.46 (d, J = 8.8 Hz, 1H), 7.67 | |
| | (t, J = 8.4 Hz, 2H), 8.03 (s, 1H), 10.10 (s, 1H), 10.98 | |
| | (s, 1H). | |
| 1-269 | 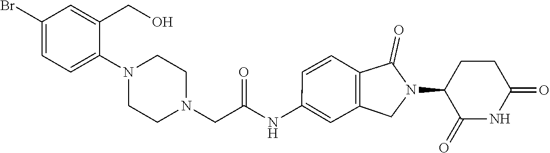 [SMILES: O=C1CC[C@H](N2Cc3cc(NC(=O)CN4CCN(c5ccc(Br)cc5CO)CC4)ccc3C2=O)C(=O)N1] | 570.2, 572.2 [M + H]+ |
| 1-270 | 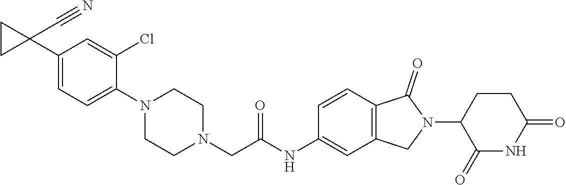 [SMILES: N#CC1(c2ccc(N3CCN(CC(=O)Nc4ccc5c(c4)CN(C4CCC(=O)NC4=O)C5=O)CC3)c(Cl)c2)CC1] | 561.4, 563.4 |
| 1-271 | 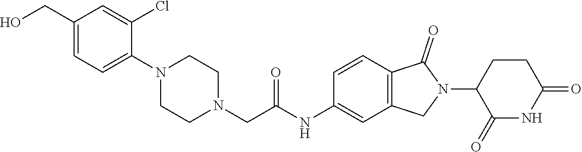 [SMILES: O=C1CCC(N2Cc3cc(NC(=O)CN4CCN(c5ccc(CO)cc5Cl)CC4)ccc3C2=O)C(=O)N1] | 526.3, 528.3 |
| 1-272 | 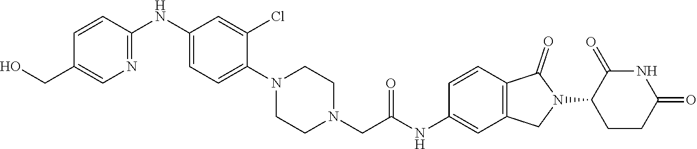 [SMILES: O=C1CC[C@H](N2Cc3cc(NC(=O)CN4CCN(c5ccc(Nc6ccc(CO)cn6)cc5Cl)CC4)ccc3C2=O)C(=O)N1] | 618.5, 620.1 |
| 1-273 | 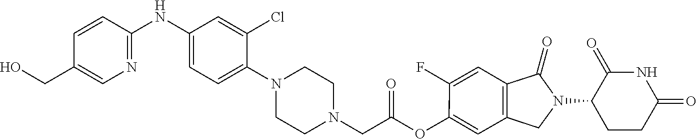 [SMILES: O=C1CC[C@H](N2Cc3cc(OC(=O)CN4CCN(c5ccc(Nc6ccc(CO)cn6)cc5Cl)CC4)c(F)cc3C2=O)C(=O)N1] | 636.3, 638.4 |
| 1-274 | 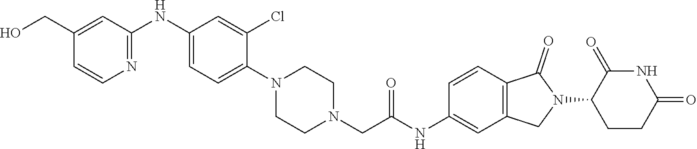 [SMILES: O=C1CC[C@H](N2Cc3cc(NC(=O)CN4CCN(c5ccc(Nc6cc(CO)ccn6)cc5Cl)CC4)ccc3C2=O)C(=O)N1] | 618.4, 620.4 |
| 1-275 | 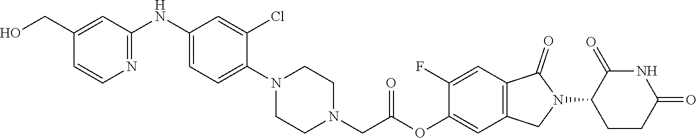 [SMILES: O=C1CC[C@H](N2Cc3cc(OC(=O)CN4CCN(c5ccc(Nc6cc(CO)ccn6)cc5Cl)CC4)c(F)cc3C2=O)C(=O)N1] | 636.4, 638.4 |
| 1-276 | 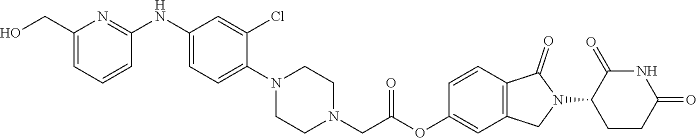 [SMILES: O=C1CC[C@H](N2Cc3cc(OC(=O)CN4CCN(c5ccc(Nc6cccc(CO)n6)cc5Cl)CC4)ccc3C2=O)C(=O)N1] | 618.4, 620.3 |
| 1-277 | 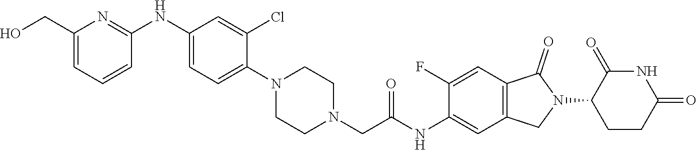 [SMILES: O=C1CC[C@H](N2Cc3cc(NC(=O)CN4CCN(c5ccc(Nc6cccc(CO)n6)cc5Cl)CC4)c(F)cc3C2=O)C(=O)N1] | 636.3, 638.3 |
| 1-278 | 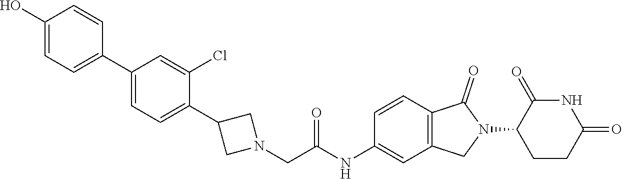 [SMILES: O=C1CC[C@H](N2Cc3cc(NC(=O)CN4CC(c5ccc(-c6ccc(O)cc6)cc5Cl)C4)ccc3C2=O)C(=O)N1] | 559.2, 561.3 |
| 1-279 | 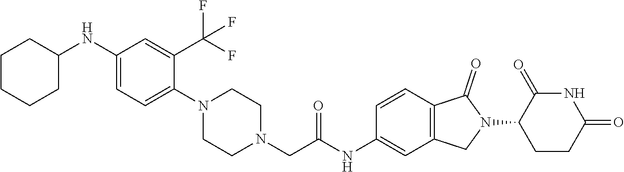 [SMILES: O=C1CC[C@H](N2Cc3cc(NC(=O)CN4CCN(c5ccc(NC6CCCCC6)cc5C(F)(F)F)CC4)ccc3C2=O)C(=O)N1] | 627.4 |
| 1-280 | 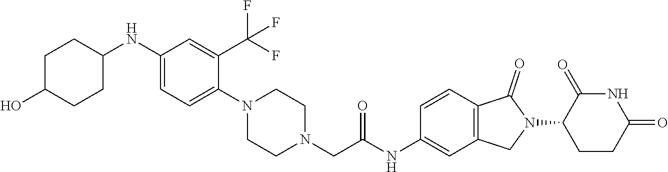 [SMILES: O=C1CC[C@H](N2Cc3cc(NC(=O)CN4CCN(c5ccc(NC6CCC(O)CC6)cc5C(F)(F)F)CC4)ccc3C2=O)C(=O)N1] | 643.4 |
| 1-281 | 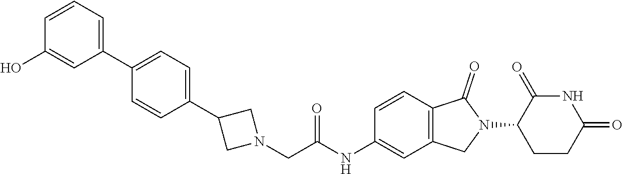 [SMILES: O=C1CC[C@H](N2Cc3cc(NC(=O)CN4CC(c5ccc(-c6cccc(O)c6)cc5)C4)ccc3C2=O)C(=O)N1] | 525.2 |
| 1-282 | 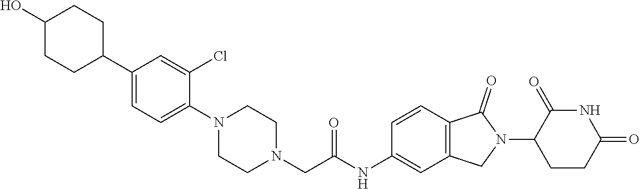 [SMILES: O=C1CCC(N2Cc3cc(NC(=O)CN4CCN(c5ccc(C6CCC(O)CC6)cc5Cl)CC4)ccc3C2=O)C(=O)N1] | 595.4, 597.4 |
| 1-283 | 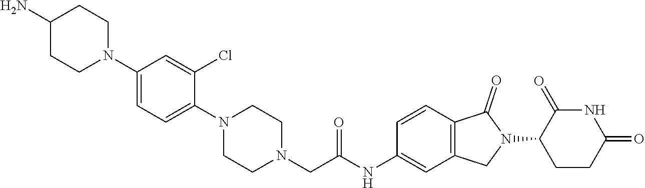 [SMILES: NC1CCN(c2ccc(N3CCN(CC(=O)Nc4ccc5c(c4)CN([C@H]4CCC(=O)NC4=O)C5=O)CC3)c(Cl)c2)CC1] | 594.3, 596.3 |
| 1-284 | 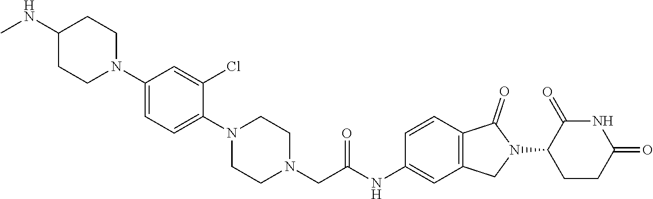 [SMILES: CNC1CCN(c2ccc(N3CCN(CC(=O)Nc4ccc5c(c4)CN([C@H]4CCC(=O)NC4=O)C5=O)CC3)c(Cl)c2)CC1] | 608.3, 610.4 |
| 1-285 | 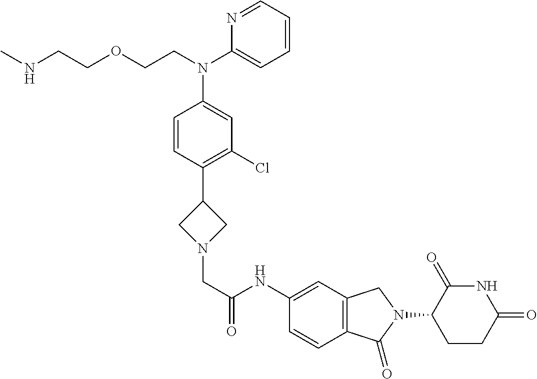 [SMILES: CNCCOCCN(c1ccc(C2CN(CC(=O)Nc3ccc4c(c3)CN([C@H]3CCC(=O)NC3=O)C4=O)C2)c(Cl)c1)c1ccccn1] | 660.4, 330.7 |
| 1-286 | 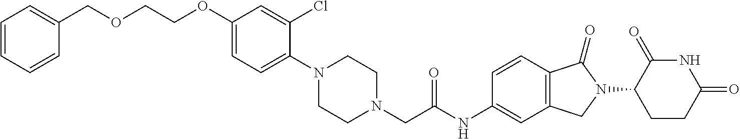 [SMILES: O=C1CC[C@H](N2Cc3cc(NC(=O)CN4CCN(c5ccc(OCCOCc6ccccc6)cc5Cl)CC4)ccc3C2=O)C(=O)N1] | 646.2, 648.2 |
| 1-287 | 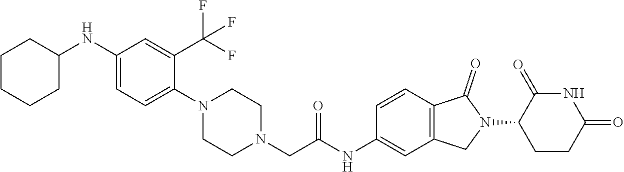 [SMILES: O=C1CC[C@H](N2Cc3cc(NC(=O)CN4CCN(c5ccc(NC6CCCCC6)cc5C(F)(F)F)CC4)ccc3C2=O)C(=O)N1] | 627.4 |
| 1-288 | 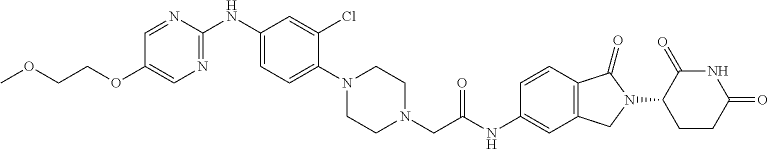 [SMILES: COCCOc1cnc(Nc2ccc(N3CCN(CC(=O)Nc4ccc5c(c4)CN([C@H]4CCC(=O)NC4=O)C5=O)CC3)c(Cl)c2)nc1] | 663.3 |
| 1-289 | 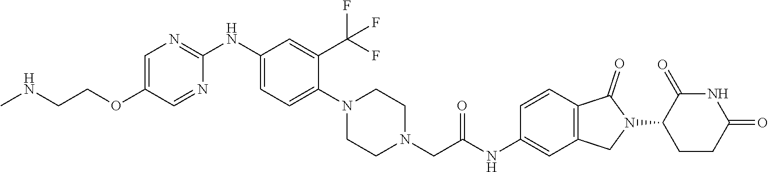 [SMILES: CNCCOc1cnc(Nc2ccc(N3CCN(CC(=O)Nc4ccc5c(c4)CN([C@H]4CCC(=O)NC4=O)C5=O)CC3)c(C(F)(F)F)c2)nc1] | 696.5 |
| 1-290 | 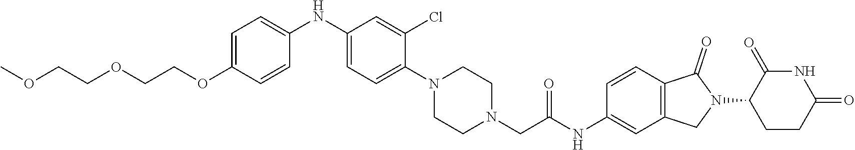 [SMILES: COCCOCCOc1ccc(Nc2ccc(N3CCN(CC(=O)Nc4ccc5c(c4)CN([C@H]4CCC(=O)NC4=O)C5=O)CC3)c(Cl)c2)cc1] | 705.3 |
| 1-291 | 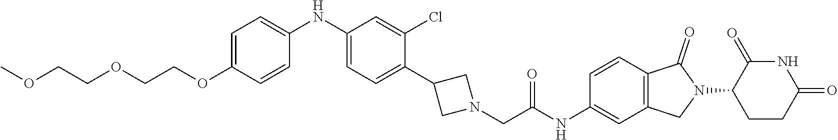 [SMILES: COCCOCCOc1ccc(Nc2ccc(C3CN(CC(=O)Nc4ccc5c(c4)CN([C@H]4CCC(=O)NC4=O)C5=O)C3)c(Cl)c2)cc1] | 676.4 |
| 1-292 | 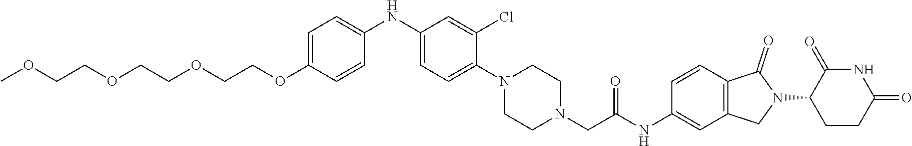 [SMILES: COCCOCCOCCOc1ccc(Nc2ccc(N3CCN(CC(=O)Nc4ccc5c(c4)CN([C@H]4CCC(=O)NC4=O)C5=O)CC3)c(Cl)c2)cc1] | 749.4 |
| 1-293 | 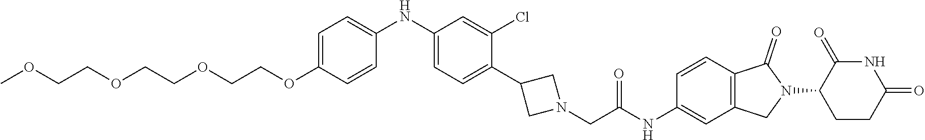 [SMILES: COCCOCCOCCOc1ccc(Nc2ccc(C3CN(CC(=O)Nc4ccc5c(c4)CN([C@H]4CCC(=O)NC4=O)C5=O)C3)c(Cl)c2)cc1] | 720.4 |
| 1-294 | 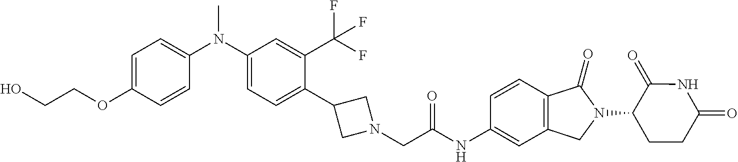 [SMILES: CN(c1ccc(OCCO)cc1)c1ccc(C2CN(CC(=O)Nc3ccc4c(c3)CN([C@H]3CCC(=O)NC3=O)C4=O)C2)c(C(F)(F)F)c1] | 666.4 |

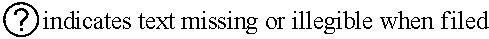

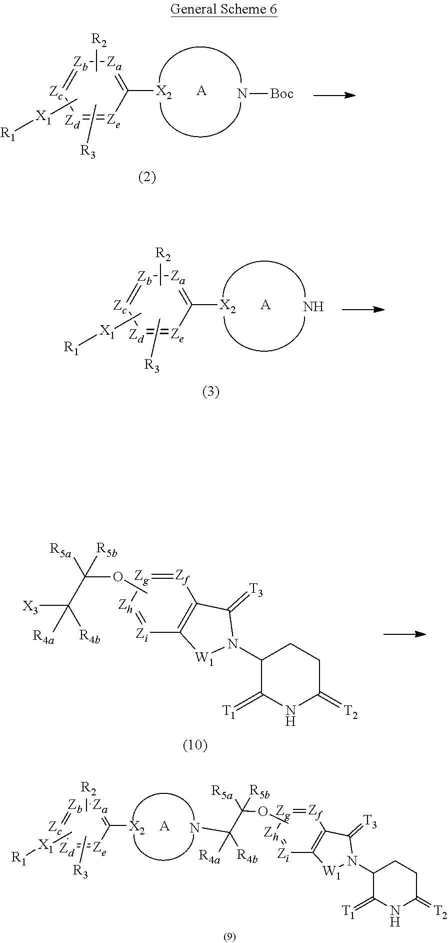

### Method 32

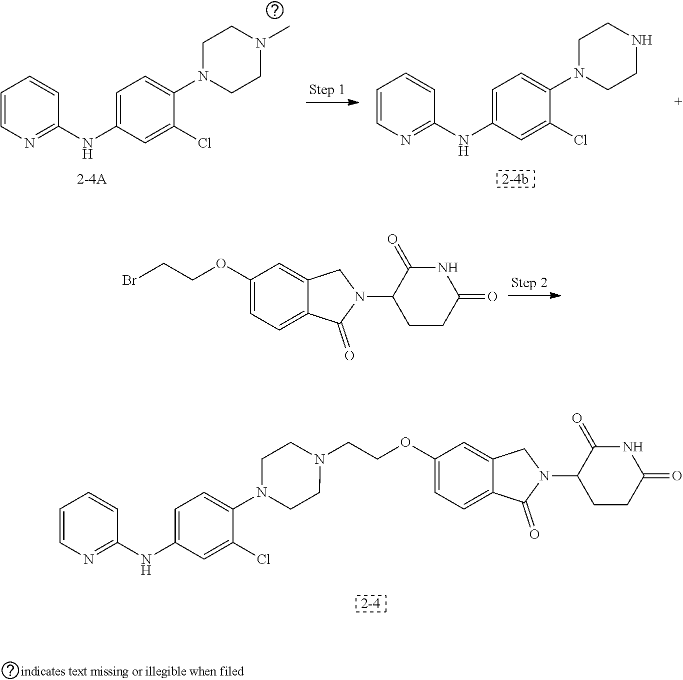 [SMILES: CN1CCN(c2ccc(Nc3ccccn3)cc2Cl)CC1 ; Clc1cc(Nc2ccccn2)ccc1N1CCNCC1 ; O=C1CCC(N2Cc3cc(OCCBr)ccc3C2=O)C(=O)N1 ; O=C1CCC(N2Cc3cc(OCCN4CCN(c5ccc(Nc6ccccn6)cc5Cl)CC4)ccc3C2=O)C(=O)N1]

Step 1: To a solution of tert-butyl 4-(2-chloro-4-(pyridin-2-ylamino)phenyl)piperazine-1-carboxylate (940 mg, 2.41 mmol) in DCM (5 mL) was added TFA (5.50 g, 48.22 mmol) dropwise at 0° C. The reaction mixture was stirred at room temperature for 2 hours. The reaction mixture was then concentrated in vacuo and purified by silica gel chromatography (DCM/MeOH=20:1 to 10:1) to give 2-4b (690 mg, 99% yield). ¹H NMR (400 MHz, DMSO_d₆) δ 10.59 (s, 1H), 9.50 (s, 2H), 8.06 (d, J=4.8 Hz, 1H), 7.91 (t, J=7.6 Hz, 1H), 7.71 (d, J=2.0 Hz, 1H), 7.42 (dd, J=8.8, 2.4 Hz, 1H), 7.26 (d, J=8.4 Hz, 1H), 7.15 (d, J=8.8 Hz, 1H), 6.96 (t, J=6.4 Hz, 1H), 3.21 (s, 8H). ESI-MS: m/z=289.0 [M+1]⁺.
Step 2: A solution of 2-4b (46.00 g, 141.44 μmol), 3-(5-(2-chloroethoxy)-1-oxoisoindolin-2-yl)piperidine-2,6-dione (50.21 mg, 155.58 μmol), and TEA (57.25 mg, 565.74 μmol) in DMF (2 mL) was stirred at 100° C. for 16 hours. The reaction mixture was then concentrated in vacuo and purified by silica gel chromatography (DCM/MeOH=30:1 to 15:1) to give 2-4 (13.8 mg, 16% yield) as a gray solid. ¹H NMR (400 MHz, DMSO_d₆) δ 10.97 (s, 1H), 9.05 (s, 1H), 8.14 (d, J=3.6 Hz, 1H), 7.96 (d, J=2.4 Hz, 1H), 7.63 (d, J=8.4 Hz, 1H), 7.58-7.51 (m, 1H), 7.45 (dd, J=8.8, 2.4 Hz, 1H), 7.21 (s, 1H), 7.12-7.04 (m, 2H), 6.82-6.68 (m, 2H), 5.08 (dd, J=13.6, 5.2 Hz, 1H), 4.39 (d, J=17.2 Hz, 1H), 4.32-4.15 (m, 3H), 3.01-2.84 (m, 5H), 2.81 (t, J=5.2 Hz, 2H), 2.74-2.53 (m, 5H), 2.45-2.30 (m, 1H), 2.04-1.93 (m, 1H). ESI-MS: m/z=575.0 [M+1]⁺.

### Method 33

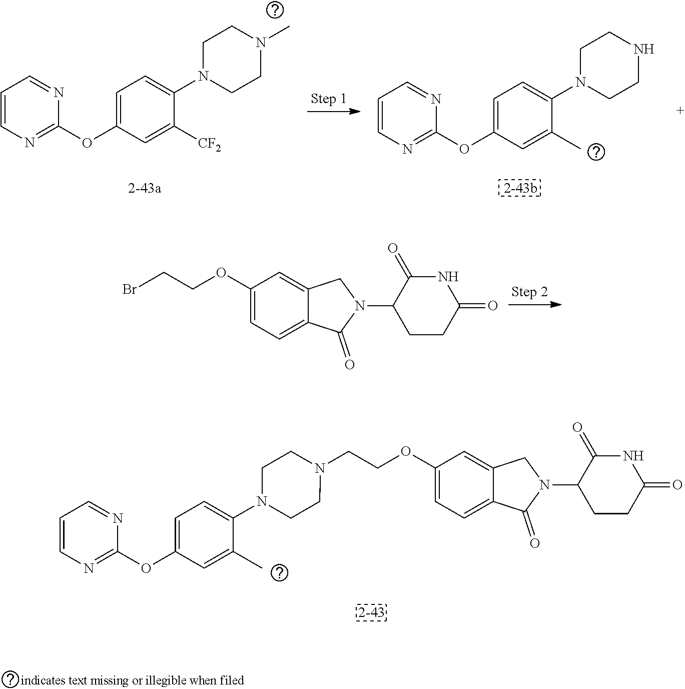

Step 1: To a solution of tert-butyl 3-(4-(pyrimidin-2-yloxy)-2-(trifluoromethyl) phenyl) azetidine-1-carboxylate (440 mg, 1.04 mmol) in DCM (5 mL) was added TFA (2.36 g, 20.73 mmol) at 0° C. The reaction mixture was stirred at room temperature for 2 hours. The reaction mixture was then concentrated in vacuo and purified by silica gel chromatography (DCM/MeOH=10:1) to obtain 2-43b (380 mg, quant.). ESI-MS: m/z=325.3 [M+1]⁺.
Step 2: A solution of 2-43b (50 mg, 114.07 μmol), 3-[5-(2-bromoethoxy)-1-oxo-isoindolin-2-yl]piperidine-2,6-dione (46.07 mg, 125.48 μmol) and TEA (46.17 mg, 456.28 μmol) in DMF (2 mL) was stirred at 50° C. for 18 hours. The reaction mixture was then concentrated in vacuo and purified by silica gel chromatography (DCM/MeOH=30:1 to 15:1) to obtain 2-43 (33.6 mg, 48% yield). ESI-MS: m/z=611.4 [M+1]⁺.

### Method 34

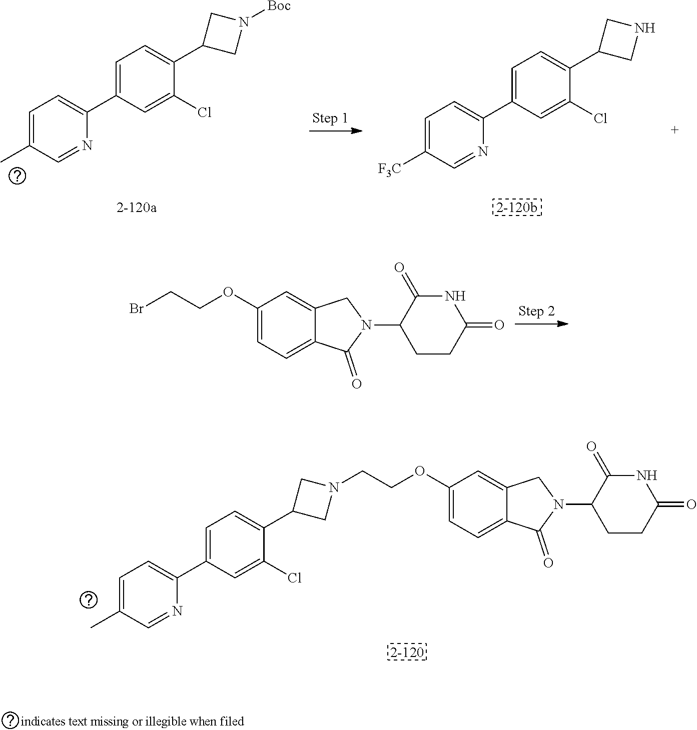 [SMILES: Cc1ccc(-c2ccc(C3CN(C(=O)OC(C)(C)C)C3)c(Cl)c2)nc1 ; FC(F)(F)c1ccc(-c2ccc(C3CNC3)c(Cl)c2)nc1 ; O=C1CCC(N2Cc3cc(OCCBr)ccc3C2=O)C(=O)N1 ; Cc1ccc(-c2ccc(C3CN(CCOc4ccc5c(c4)CN(C4CCC(=O)NC4=O)C5=O)C3)c(Cl)c2)nc1]

Step 1: A solution of tert-butyl 3-[2-chloro-4-[5-(trifluoromethyl)-2-pyridyl]-phenyl]azetidine-1-carboxylate (70 mg, 169.56 μmmol) and TFA (386.67 mg, 3.39 mmol) in DCM (1 mL) was stirred at room temperature for 2 hours. The mixture was concentrated to afford a crude product. The resulting solid was stirred in MTBE (10 mL) for 6 hours. The mixture was filtered, and the filter cake was dried to dryness to give 2-120b (72.00 mg, quant.) as a gray solid. ESI-MS: m/z=313.2, 315.1 [M+1]⁺.
Step 2: A solution of 2-120b (72.00 mg, 168.72 μmol), 3-(5-(2-bromoethoxy)-1-oxoisoindolin-2-yl)piperidine-2,6-dione (68.15 mg, 185.59 μmol) and TEA (102.44 mg, 1.01 mmol) in DMF (2 mL) was stirred at 50° C. for 18 hours. The reaction mixture was then concentrated in vacuo and purified by silica gel chromatography (DCM/MeOH=30:1 to 15:1) to obtain 2-120 (100 mg, 98% yield). ESI-MS: m/z=599.3, 601.3 [M+1]⁺.

### Method 35

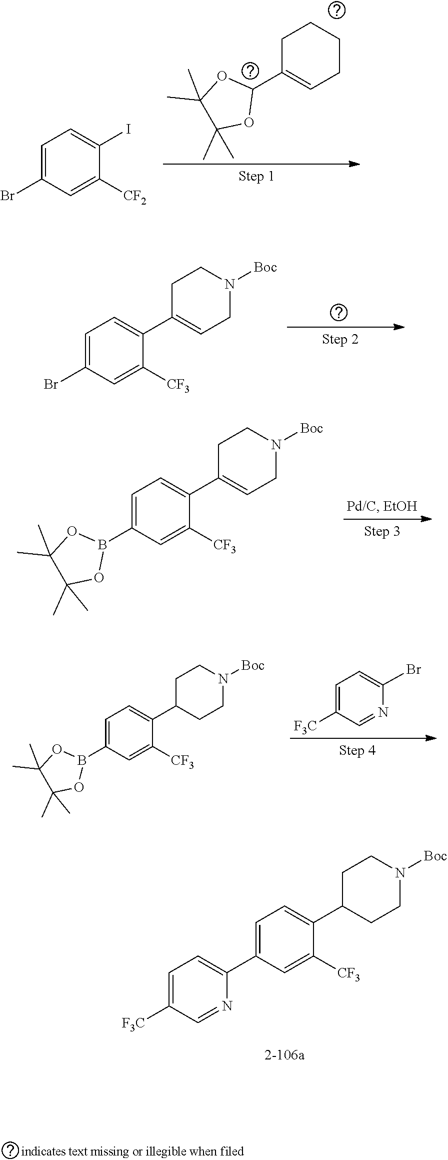

Step 1: To a solution of 4-bromo-1-iodo-2-(trifluoromethyl)benzene (10.00 g, 28.50 mmol) in 1,4-dioxane/H₂O (5/1, 120 mL) was added tert-butyl 4-(4,4,5,5-tetramethyl-1,3,2-dioxaborolan-2-yl)-3,6-dihydropyridine-1(2H)-carboxylate (8.90 g, 28.50 mmol), Pd(PPh₃)₄ (1.65 g, 1.40 mmol), and K₂CO₃ (11.82 g, 85.50 mmol). The reaction mixture was stirred at 80° C. for 16 hours under N₂. The reaction mixture was poured into water (500 mL) and extracted with EtOAc (150 mL×3). The combined organic layers were washed with brine, dried over Na₂SO₄, and concentrated to afford a crude product. The crude product was purified by silica gel chromatography (PE/EtOAc=20/1˜5/1) to give tert-butyl 4-(4-bromo-2-(trifluoromethyl)phenyl)-3,6-dihydropyridine-1(2H)-carboxylate (3.50 g, 30.2%) as a yellow oil. ESI-MS: m/z=349.9, 351.9 [M+1]⁺.
Step 2: A solution of tert-butyl 4-(4-bromo-2-(trifluoromethyl)phenyl)-3,6-dihydropyridine-1(2H)-carboxylate (3.00 g, 7.40 mmol), bis(pinacolato)diboron (1.88 g, 7.40 mmol), Pd(dppf)Cl₂ (0.27 g, 0.30 mmol), and KOAc (2.18 g, 22.20 mmol) in 1,4-dioxane (50 mL) was stirred under nitrogen at 85° C. for 12 hours. The reaction mixture was concentrated to afford a crude product. The crude product was purified by prep-TLC (PE/EtOAc=10/1) to give tert-butyl 4-(4-(4,4,5,5-tetramethyl-1,3,2-dioxaborolan-2-yl)-2-(trifluoromethyl)phenyl)-3,6-dihydropyridine-1(2H)-carboxylate (2.20 g, 65.7%) as a white solid. ¹H NMR (400 MHz, DMSO_d₆) δ 7.89 (d, J=8.0 Hz, 2H), 7.41 (d, J=7.6 Hz, 1H), 5.59 (s, 1H), 3.94 (s, 2H), 3.52 (t, J=5.2 Hz, 2H), 2.28 (s, 2H), 1.43 (s, 9H), 1.31 (s, 12H).
Step 3: A solution of tert-butyl 4-(4-(4,4,5,5-tetramethyl-1,3,2-dioxaborolan-2-yl)-2-(trifluoromethyl)phenyl)-3,6-dihydropyridine-1(2H)-carboxylate (2.00 g, 0.40 mmol) and 10% Pd/C (0.50 g) in EtOH (20 mL) was stirred under H₂ balloon at 40° C. for 16 hours. The mixture was filtered. The filtrate was concentrated to give tert-butyl 4-(4-(4,4,5,5-tetramethyl-1,3,2-dioxaborolan-2-yl)-2-(trifluoromethyl)phenyl)piperidine-1-carboxylate (1.50 g, 74.7%) as a white solid. ¹H NMR (400 MHz, DMSO-d₆) δ 7.88 (d, J=8.0 Hz, 2H), 7.66 (d, J=8.0 Hz, 1H), 4.09 (s, 2H), 3.47-3.40 (m, 1H), 1.69-1.55 (m, 4H), 1.42 (s, 9H), 1.29 (s, 12H), 1.06 (dt, J=11.9, 5.8 Hz, 2H). ESI-MS: m/z=349.9, 351.9 [M+1]⁺.
Step 4: A solution of tert-butyl 4-(4-(4,4,5,5-tetramethyl-1,3,2-dioxaborolan-2-yl)-2-(trifluoromethyl)phenyl)piperidine-1-carboxylate (300.0 mg, 0.66 mmol), 2-bromo-5-(trifluoromethyl)pyridine (193.1 mg, 0.85 mmol), Pd(dppf)Cl₂ (24.1 mg, 0.03 mmol), and potassium carbonate (272.6 mg, 1.97 mmol) in 1,4-dioxane/H₂O (5/1, 10 mL) was stirred under nitrogen at 85° C. for 16 hours. The reaction mixture was poured into water (10 mL) and extracted with EtOAc (5 mL×3). The combined organic layers were washed with brine, dried over Na₂SO₄, and concentrated to afford a crude product. The crude product was purified by prep-TLC (PE/EtOAc=5/1) to give tert-butyl 4-(2-(trifluoromethyl)-4-(5-(trifluoromethyl)pyridin-2-yl)phenyl)piperidine-1-carboxylate (2-106a, 200.0 mg, 64.0%) as a white solid. ¹H NMR (400 MHz, DMSO-d₆) δ 9.08 (s, 1H), 8.46 (d, J=1.6 Hz, 1H), 8.40 (d, J=8.0 Hz, 1H), 8.33 (d, J=1.6 Hz, 2H), 7.83 (d, J=8.4 Hz, 1H), 4.12 (s, 2H), 3.04 (s, 1H), 2.83 (s, 2H), 1.77-1.62 (m, 4H), 1.43 (s, 9H). ESI-MS: m/z=419.2 [M−56+1]⁺.

### Method 36

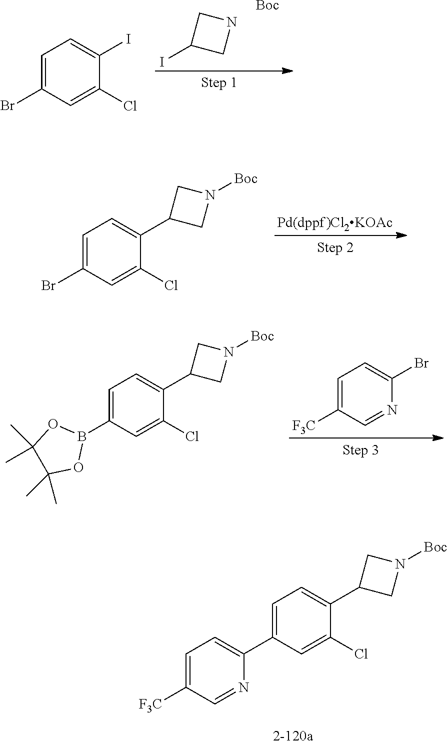

Step 1: To a stirred solution of Zn (9.0 g, 0.14 mol) in THF (150 mL) was added 1,2-Dibromoethane (3.17 g, 16.8 mmol). The reaction mixture was stirred at 80° C. for 10 minutes. Then the reaction mixture was cooled to room temperature and chlorotrimethylsilane (1.72 g, 15.8 mmol) in THF (60 mL) was slowly added dropwise. The reaction was stirred at 25° C. for 4 minutes. Then tert-butyl 3-iodoazetidine-1-carboxylate (30.0 g, 0.11 mol) in THF (150 mL) was slowly added dropwise. The reaction was stirred at 25° C. for 2 hours. Then 4-bromo-2-chloro-1-iodobenzene (40.2 g, 0.13 mol) in THF (540 mL) was slowly added dropwise followed by tris (dibenzylideneacetone) dipalladium (1.55 g, 1.60 mmol) and Tri (2-furyl) phosphine (1.4 g, 6.10 mmol). The reaction was stirred at 55° C. for 3 hours. The reaction mixture was poured into water (1 L) and extracted with EtOAc (1 L×3). The combined organic layers were washed with brine, dried over Na₂SO₄, and concentrated to afford a crude product. The crude product was purified by silica gel chromatography (PE/EtOAc=100/1˜20/1) to give the tert-butyl 3-(4-bromo-2-chlorophenyl) azetidine-1-carboxylate (17.5 g, 39.0% yield) as a white solid. ¹H NMR (400 MHz, DMSO_d₆) δ 7.72 (d, J=1.2 Hz, 1H), 7.57 (d, J=8.0 Hz, 1H), 7.49 (d, J=8.4 Hz, 1H), 4.23 (d, J=6.8 Hz, 2H), 4.02 (m, 1H), 3.95-3.86 (m, 2H), 1.38 (s, 9H). ESI-MS: m/z=289.9 [M+1−56]⁺.
Step 2: To a solution of tert-butyl 3-(4-bromo-2-chlorophenyl) azetidine-1-carboxylate (1.00 g, 2.9 mmol) in DMF (10 mL) was added KOAc (0.85 g, 8.7 mmol), Pd(dppf)Cl₂ (0.2 g), and Bis(pinacolato)diboron (2.21 g, 8.7 mmol). The reaction mixture was stirred at 85° C. for 2 hours under N₂. The reaction mixture was poured into water (50 mL) and extracted with EtOAc (50 mL×3). The combined organic layers were washed with brine, dried over Na₂SO₄, and concentrated to afford a crude product. The crude product was purified by prep-TLC (PE/EtOAc=5/1) to give tert-butyl 3-(2-chloro-4-(4,4,5,5-tetramethyl-1,3,2-dioxaborolan-2-yl) phenyl) azetidine-1-carboxylate (0.8 g, yield 68.3%) as a yellow solid. ¹H NMR (400 MHz, DMSO_d₆) δ 7.65-7.55 (m, 3H), 4.27 (t, J=8.0 Hz, 2H), 4.13-4.05 (m, 1H), 3.91 (t, J=7.2 Hz, 2H), 1.39 (s, 9H), 1.29 (s, 12H). ESI-MS: m/z=338.0 [M+1−56]⁺.
Step 3: To a solution of tert-butyl 3-(2-chloro-4-(4,4,5,5-tetramethyl-1,3,2-dioxaborolan-2-yl) phenyl) azetidine-1-carboxylate (500.0 mg, 1.3 mmol) in 1,4-dioxane/H₂O=3/1 (8 mL) was added 2-bromo-5-(trifluoromethyl) pyridine (287.0 mg, 1.3 mmol), K₂CO₃ (520.0 mg, 3.8 mmol), and Pd(dppf)Cl₂ (100.0 mg). The reaction mixture was stirred at 85° C. for 12 hours under N₂. The reaction mixture was poured into water (50 mL) and extracted with EtOAc (30 mL×3). The combined organic layers were washed with brine, dried over Na₂SO₄, and concentrated to afford a crude product. The crude product was purified by prep-TLC (PE/EtOAc=5/1) to give tert-butyl 3-(2-chloro-4-(5-(trifluoromethyl) pyridin-2-yl) phenyl) azetidine-1-carboxylate (2-120a, 0.20 g, yield 35.07%) as a yellow solid. ¹H NMR (400 MHz, DMSO_d₆) δ 9.06 (s, 1H), 8.35-8.25 (m, 3H), 8.18 (dd, J=8.2, 1.6 Hz, 1H), 7.72 (d, J=8.2 Hz, 1H), 4.32-4.28 (m, 2H), 4.18-4.10 (m, 1H), 4.00-3.97 (m, 2H), 1.40 (s, 9H). ESI-MS: m/z=357.1 [M+1−56]⁺.

### Synthesis of Diarylamine Intermediate of Compound 2-169a:

### Method 37

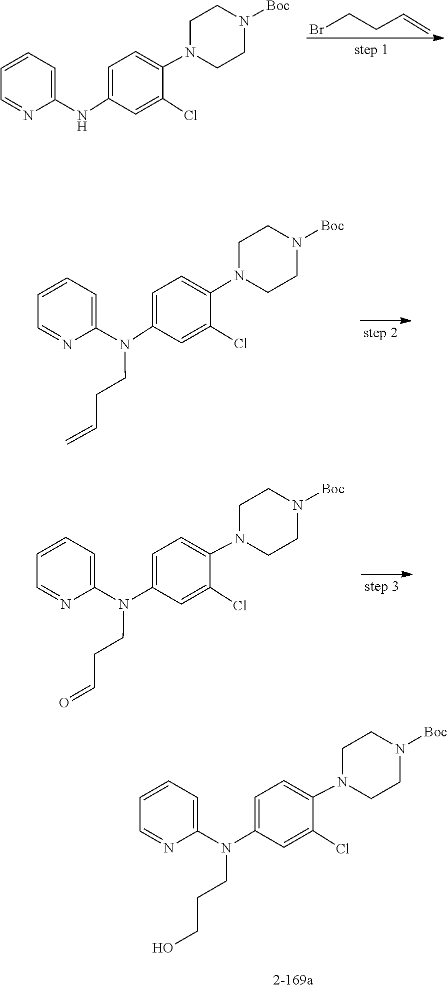

Step 1: To a stirred solution of tert-butyl 4-(2-chloro-4-(pyridin-2-ylamino)phenyl)piperazine-1-carboxylate (1 g, 2.60 mmol) in DMF (10 mL) was added NaH (0.52 g, 130 mmol) dropwise at 0° C. The mixture was stirred at 25° C. for 3 hours. Then 4-bromobut-1-ene (0.42 g, 3.11 mmol) was added. The reaction was stirred 25° C. for 2 hours. The reaction mixture was poured into water (30 mL) and extracted with EtOAc (3×15 mL). The combined organic layers were washed with brine, dried over Na₂SO₄ and concentrated to afford a crude product. The crude product was purified by silica gel chromatography (PE/EtOAc=20/1˜5/1) to give tert-butyl 4-(4-(but-3-en-1-yl(pyridin-2-yl)amino)-2-chlorophenyl)piperazine-1-carboxylate (0.80 g, yield 70.23%) as a yellow oil. ¹H NMR (400 MHz, DMSO_d₆) δ 1.37 (s, 9H), 2.72 (s, 3H), 2.82 (t, J=6.0 Hz, 2H), 3.26 (t, J=5.6 Hz, 2H), 3.46 (t, J=5.6 Hz, 2H), 3.61 (t, J=6.4 Hz, 2H), 7.42 (d, J=8.0 Hz, 1H), 7.73 (dd, J=8.0, 2.4 Hz, 1H), 8.27 (s, 1H).
Step 2-3: To a stirred solution of tert-butyl 4-(4-(but-3-en-1-yl(pyridin-2-yl)amino)-2-chlorophenyl)piperazine-1-carboxylate (700 mg, 1.58 mmol) in THF/H₂O (3/1, 10 mL) was added potassium osmate(VI) dihydrate (11.6 mg, 0.032 mmol) at 0° C. The mixture was stirred at 0° C. for 15 minutes. Then NaIO₄ (1.01 g, 4.74 mmol) was added. The reaction was stirred at 25° C. for 4 hours. The mixture was filtered. The filtrate was concentrated and poured into water (100 mL) and extracted with EtOAc (100 mL×3). The combined organic layers were washed with brine, dried over Na₂SO₄ and concentrated to give tert-butyl 4-(2-chloro-4-((3-oxopropyl)(pyridin-2-yl)amino)phenyl)piperazine-1-carboxylate. A solution of tert-butyl 4-(2-chloro-4-((3-oxopropyl)(pyridin-2-yl)amino)phenyl)piperazine-1-carboxylat, NaBH₄ (90 mg, 2.38 mmol) in EtOH (10 mL) was stirred under nitrogen at 25° C. for 15 minutes. The mixture was filtered. The filtrate was concentrated to afford a crude product. The crude product was purified by prep-TLC (PE/EtOAc=3/1) to give tert-butyl 4-(2-chloro-4-((3-hydroxypropyl)(pyridin-2-yl)amino)phenyl)piperazine-1-carboxylate (2-169a, 300 mg, yield 42.66%) as a yellow oil. ESI-MS: m/z=447.3 [M+1]⁺.

### Synthesis of Diarylamine Intermediate of Compound 2-175a:

### Method 38

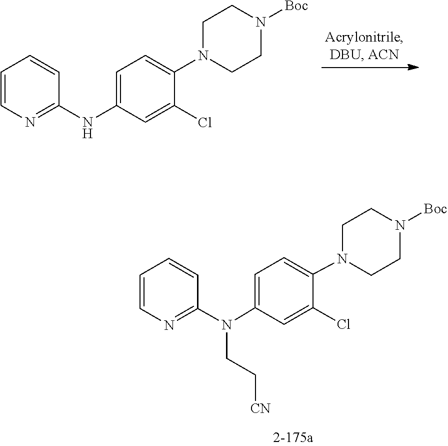 [SMILES: CC(C)(C)OC(=O)N1CCN(c2ccc(Nc3ccccn3)cc2Cl)CC1 ; CC(C)(C)OC(=O)N1CCN(c2ccc(N(CCC#N)c3ccccn3)cc2Cl)CC1]

A solution of tert-butyl 4-[2-chloro-4-(2-pyridylamino)phenyl]piperazine-1-carboxylate (250 mg, 642.86 μmmol) in ACN (5 mL) was stirred at room temperature. Acrylonitrile (341.10 mg, 6.43 mmol), DBU (195.74 mg, 1.29 mmol) added and stirred at 80° C. for 2 days. The reaction mixture was poured into water (100 mL) and extracted with EtOAc (100 mL×3). The combined organic layers were washed with brine, dried over Na₂SO₄ and concentrated to afford a crude product. The crude product was purified by silica gel chromatography (EtOAc/Hexane=1:5) to give tert-butyl 4-[2-chloro-4-[2-cyanoethyl(2-pyridyl)amino]phenyl]piperazine-1-carboxylate (2-175a, 180 mg, 63.3%). ESI-MS: m/z=442.1, 444.1 [M+1]⁺.

### Synthesis of Diarylamine Intermediate of Compound 2-187a

### Method 39

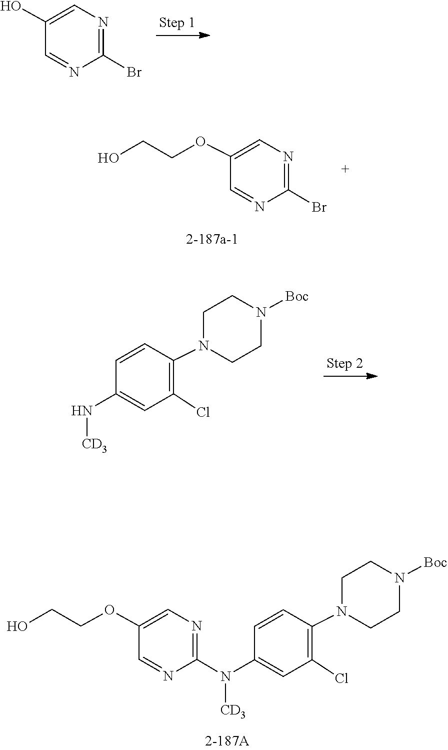 [SMILES: Oc1cnc(Br)nc1 ; OCCOc1cnc(Br)nc1 ; [2H]C([2H])([2H])Nc1ccc(N2CCN(C(=O)OC(C)(C)C)CC2)c(Cl)c1 ; [2H]C([2H])([2H])N(c1ccc(N2CCN(C(=O)OC(C)(C)C)CC2)c(Cl)c1)c1ncc(OCCO)cn1]

Step 1: To a mixture of 2-bromopyrimidin-5-ol (2.00 g, 11.4 mmol) and potassium carbonate (3.16 g, 22.9 mmol) in acetonitrile (40 mL) was added 2-bromoethanol (0.89 mL, 12 mmol). The mixture was heated to 60° C. with stirring for 3 hours, cooled to room temperature, poured over H₂O, and extracted with EtOAc. The combined organic layers were washed with brine, dried over Na₂SO₄, filtered, and concentrated. The residue was recrystallized from a minimum of CH₂Cl₂. 2-187a-1 was collected as off-white crystals in 40% yield (1.01 g, 4.61 mmol). The filtrate was concentrated, and the residue was purified by FCC on SiO₂ using a gradient of 50-100% EtOAc in hexanes. An additional 26% (642 mg, 2.93 mmol) was collected as a white crystalline solid. Total yield: 66% (1.65 g, 7.53 mmol). TLC (EtOAc): R_{f}=0.44. LC/MS found 219.0 [M+H]⁺.
Step 2: A mixture of 2-(2-bromopyrimidin-5-yl)oxyethanol (1.01 g, 4.61 mmol), tert-butyl 4-[2-chloro-4-(trideuteriomethylamino)phenyl]piperazine-1-carboxylate (1.59 g, 4.84 mmol), methanesulfonato(2-dicyclohexylphosphino-2′,6′-di-i-propoxy-1,1′-biphenyl)(2′-methylamino-1,1′-biphenyl-2-yl)palladium(II) (77 mg, 91 μmol), 2-dicyclohexylphosphino-2′,6′-di-i-propoxy-1,1′-biphenyl (43 mg, 92 μmol), and cesium carbonate (3.00 g, 9.21 mmol) in dry 1,4-dioxane (20 mL) was degassed via sparging with N₂ for 10 minutes then heated to 80° C. with stirring for 4 hours. The mixture was cooled to room temperature and filtered through a pad of diatomaceous earth, and the filtrate was concentrated to dryness. The residue was purified by flash column chromatography using a gradient of 50-100% EtOAc in hexanes. 2-187a was collected as a yellow semi-solid with 95% purity in 56% yield (1.27 g, 2.58 mmol). LC/MS found 467.3 [M+H]⁺.

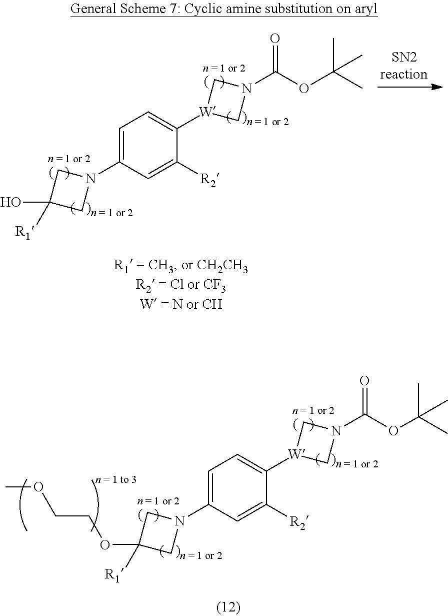

### Synthesis of Compound 2-163a

### Method 40

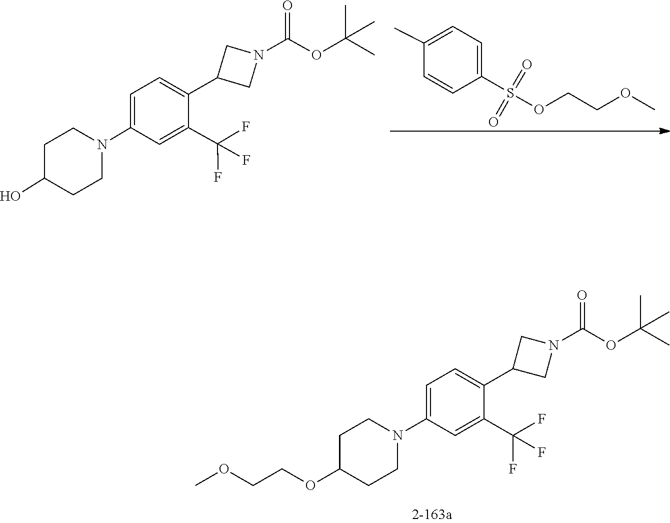

A mixture of tert-butyl 3-[4-(4-hydroxy-1-piperidyl)-2-(trifluoromethyl)phenyl]azetidine-1-carboxylate (67 mg, 167.32 μmol), 2-methoxyethyl 4-methylbenzenesulfonate (77.06 mg, 334.64 μmol, 70.05 μL), and NaH (60% dispersion in oil) (20.08 mg, 501.96 μmol, 60% purity) in DMF (1.93 mL) was heated to 70° C. and shaken for overnight. The mixture was cooled to a room temperature and diluted with EtOAc (˜30 mL) and brine (˜20 mL). The organic phase was separated, washed with brine (˜20 mL), dried over sodium sulfate, filtered, concentrated, and purified by a flash column to afford tert-butyl 3-[4-[4-(2-methoxyethoxy)-1-piperidyl]-2-(trifluoromethyl)phenyl]azetidine-1-carboxylate (2-163a, 30 mg, 65.43 μmol, 39.10% yield). EtOAc (10% to 100%) in Hexane. ESI-MS: m/z=459.3 [M+1]⁺

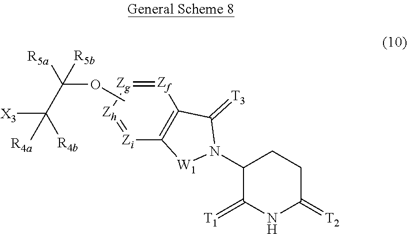

### Synthesis of Ether Linker Intermediate

### Method 41

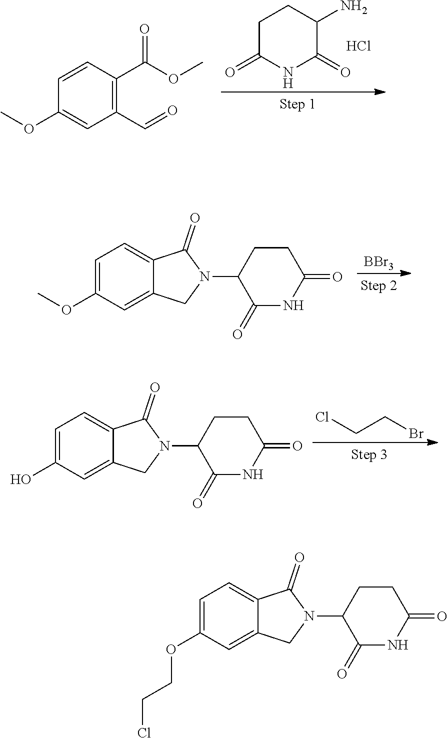

Step 1: To a stirred solution of methyl 2-formyl-4-methoxybenzoate (20.00 g, 103.00 mmol) in DCM (200 mL) was added 3-aminopiperidine-2,6-dione hydrochloride (20.34 g, 123.60 mmol) and AcOH (10 mL). The mixture was stirred at 40° C. for 12 hours. Then Sodium cyanoborohydride (9.71 g, 154.50 mmol) was added. The reaction was stirred 40° C. for 4 hours. The reaction mixture was poured into water (150 mL) and extracted with EtOAc (50 mL×3). The combined organic layers were washed with brine, dried over Na₂SO₄, and concentrated to afford a crude product. The crude product was purified by silica gel chromatography (PE/EtOAc=20/1˜1/1) to give 3-(5-methoxy-1-oxoisoindolin-2-yl)piperidine-2,6-dione (12.00 g, 42.5% yield) as white solid. ¹H NMR (400 MHz, DMSO_d₆) δ 10.88 (s, 1H), 7.54 (d, J=8.4 Hz, 1H), 7.08 (d, J=2.0 Hz, 1H), 6.96 (dd, J=8.4, 2.0 Hz, 1H), 4.98 (dd, J=13.2, 5.2 Hz, 1H), 4.33 (dd, J=51.2, 17.2 Hz, 1H), 3.75 (s, 3H), 2.96-2.84 (m, 1H), 2.54-2.46 (m, 1H), 2.43-2.31 (m, 1H), 1.94-1.85 (m, 1H).
Step 2: A solution of 3-(5-methoxy-1-oxoisoindolin-2-yl)piperidine-2,6-dione (7.00 g, 25.50 mmol) and BBr₃ (63.88 g, 255.00 mmol) in DCM (100 mL) was stirred at 25° C. for 12 hours. The reaction mixture was concentrated to give 3-(5-hydroxy-1-oxoisoindolin-2-yl)piperidine-2,6-dione (5.00 g, 75.3% yield) as a yellow solid. ESI-MS: m/z=261.1 [M+1]⁺.
Step 3: A solution of 3-(5-hydroxy-1-oxoisoindolin-2-yl)piperidine-2,6-dione (2.50 g, 9.60 mmol), 1-Bromo-2-chloroethane (1.65 g, 11.50 mmol) and K₃PO₄ (3.06 g, 14.40 mmol) in DMSO (50 mL) was stirred at 60° C. for 3 hours. The reaction mixture was poured into water (250 mL) and extracted with EtOAc (50 mL×3). The combined organic layers were washed with brine, dried over Na₂SO₄, and concentrated to afford a crude product. The residue was purified by prep-HPLC and lyophilized to give 3-(5-(2-chloroethoxy)-1-oxoisoindolin-2-yl)piperidine-2,6-dione (0.35 g, 11.3% yield) as an off-white solid. ¹H NMR (400 MHz, DMSO_d₆) δ 10.94 (s, 1H), 7.64 (d, J=8.4 Hz, 1H), 7.21 (d, J=2.0 Hz, 1H), 7.09 (dd, J=8.4, 2.0 Hz, 1H), 5.08 (dd, J=13.2, 5.2 Hz, 1H), 4.44-4.24 (m, 4H), 4.22 (d, J=17.2, 1H), 2.97-2.85 (m, 1H), 2.63-2.55 (m, 1H), 2.43-2.32 (m, 1H), 2.03-1.95 (m, 1H). ESI-MS: m/z=323.1 [M+1]⁺.

### Method 42

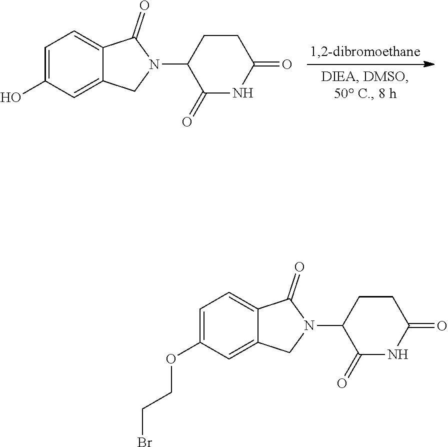 [SMILES: O=C1CCC(N2Cc3cc(O)ccc3C2=O)C(=O)N1 ; O=C1CCC(N2Cc3cc(OCCBr)ccc3C2=O)C(=O)N1]

A solution of 3-(5-hydroxy-1-oxoisoindolin-2-yl)piperidine-2,6-dione (5 g, 19.20 mmol), DIEA (17.37 g, 134.40 mmol), and 1,2-dibromoethane (13.77 g, 95.90 mmol) in DMSO (50 mL) was stirred at 50° C. for 8 hours. The reaction mixture was poured into water (250 mL) and extracted with EtOAc (150 mL×3). The combined organic layers were washed with brine, dried over Na₂SO₄, and concentrated to afford a crude product. The residue was purified by prep-HPLC and lyophilized to give 3-(5-(2-bromoethoxy)-1-oxoisoindolin-2-yl)piperidine-2,6-dione (0.70 g, 11.3% yield) as a yellow solid. ¹H NMR (400 MHz, DMSO_d₆) δ 10.97 (s, 1H), 7.64 (d, J=8.4 Hz, 1H), 7.21 (s, 1H), 7.09 (d, J=8.4 Hz, 1H), 5.08 (dd, J=13.2, 5.2 Hz, 1H), 4.46-4.23 (m, 4H), 4.04-3.95 (m, 2H), 2.98-2.85 (m, 1H), 2.69-2.55 (m, 1H), 2.39-2.31 (m, 1H), 2.04-1.95 (m, 1H). ESI-MS: m/z=323.0/325.0 [M+1]⁺.

### Method 43

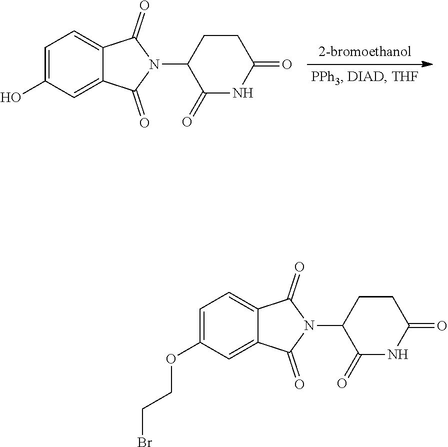 [SMILES: O=C1CCC(N2C(=O)c3ccc(O)cc3C2=O)C(=O)N1 ; O=C1CCC(N2C(=O)c3ccc(OCCBr)cc3C2=O)C(=O)N1]

To a solution of 2-(2,6-dioxopiperidin-3-yl)-5-hydroxyisoindole-1,3-dione (0.68 g, 2.47 mmol) in THF (20 mL) was added 2-bromoethanol (0.47 g, 3.75 mmol), PPh₃ (0.98 g, 3.75 mmol), and DIAD (0.76 g, 3.75 mmol) at 0° C. The reaction mixture was stirred for 5 hours at room temperature. The reaction mixture was poured into water (50 mL) and extracted with EtOAc (50 mL×3). The combined organic layers were washed with brine, dried over Na₂SO₄, and concentrated to give 5-(2-bromoethoxy)-2-(2,6-dioxopiperidin-3-yl)isoindoline-1,3-dione crude product (0.80 g, 84.6% yield). ESI-MS: m/z=381.3 [M+1]⁺.

### Method 44

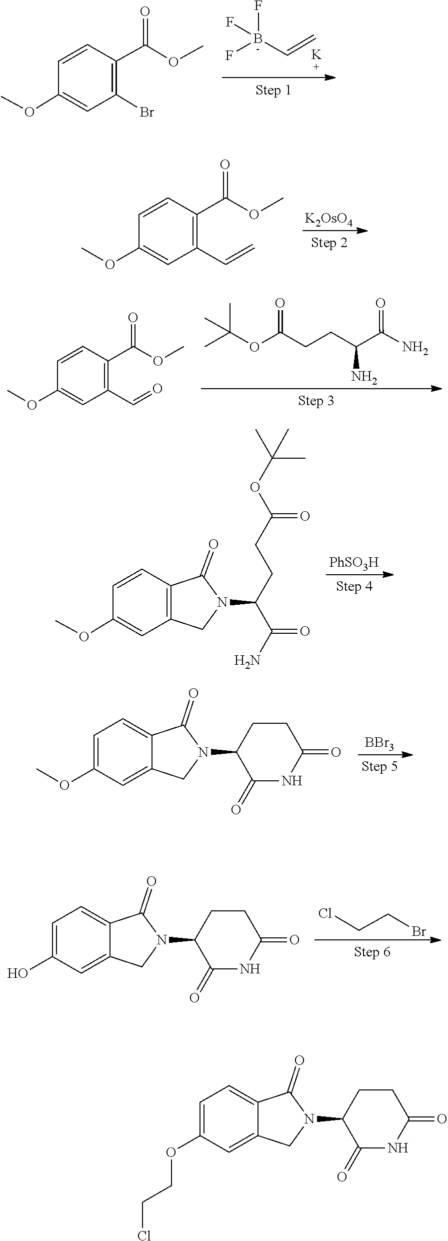

Step 1: A solution of methyl 2-bromo-4-methoxybenzoate (25.00 g, 102.00 mmol), trifluoro(vinyl)borate (15.03 g, 112.20 mmol), Pd(dppf)Cl₂ (3.73 g, 5.10 mmol), and Cs₂CO₃ (28.19 g, 204.00 mmol) in DMF (200 mL) was stirred under nitrogen at 90° C. for 16 hours. The reaction mixture was poured into water (1 L) and extracted with EtOAc (300 mL×3). The combined organic layers were washed with brine, dried over Na₂SO₄, and evaporated to dryness to give a yellow oil. The crude product was purified by silica gel chromatography (PE/EtOAc=20/1˜5/1) to give methyl 4-methoxy-2-vinylbenzoate (15.00 g, 76.5% yield) as a yellow solid. ESI-MS: m/z=193.2 [M+1]⁺.
Step 2: To a stirred solution of methyl 4-methoxy-2-vinylbenzoate (13.00 g, 67.60 mmol) in THF/H₂O (3/1, 260 mL) was added potassium osmate (VI) dihydrate (0.41 g, 1.30 mmol) dropwise at 25° C. The mixture was stirred at 25° C. for 15 minutes. Then sodium periodate (43.38 g, 202.70 mmol) was added. The reaction was stirred at 25° C. for 4 hours. The mixture was filtered, and the filtrate poured into water (100 mL) and extracted with EtOAc (100 mL×3). The combined organic layers were washed with brine, dried over Na₂SO₄, and concentrated to afford a crude product. The crude product was purified by silica gel chromatography (PE/EtOAc=20/1˜5/1) to give methyl 2-formyl-4-methoxybenzoate (7.00 g, 53.3% yield) as an off-white solid. ¹H NMR (400 MHz, DMSO_d₆) δ 10.46 (s, 1H), 7.93 (d, J=8.4 Hz, 1H), 7.31-7.26 (m, 2H), 3.88 (s, 3H), 3.87 (s, 3H).
Step 3: To a stirred solution of methyl 2-formyl-4-methoxybenzoate (5.00 g, 25.70 mmol) in DCM (50 mL) was added tert-butyl (S)-4,5-diamino-5-oxopentanoate hydrochloride (7.36 g, 30.80 mmol) and AcOH (2.5 mL) at 40° C. The mixture was stirred at 40° C. for 12 hours. Then sodium cyanoborohydride (2.42 g, 38.50 mmol) was added. The reaction was stirred at 40° C. for 4 hours. The reaction mixture was poured into water (150 mL) and extracted with EtOAc (50 mL×3). The combined organic layers were washed with brine, dried over Na₂SO₄, and concentrated to afford a crude product. The crude product was purified by silica gel chromatography (PE/EtOAc=20/1˜1/1) to give tert-butyl (S)-5-amino-4-(5-methoxy-1-oxoisoindolin-2-yl)-5-oxopentanoate (2.50 g, 27.9% yield) as a white solid. ESI-MS: m/z=349.2 [M+1]⁺, 293.1 [M−56+1]⁺.
Step 4: A solution of tert-butyl (S)-5-amino-4-(5-methoxy-1-oxoisoindolin-2-yl)-5-oxopentanoate (2.00 g, 5.70 mol) and 4-methylbenzenesulfonic acid (5.88 g, 34.20 mmol) in ACN (20 mL) was stirred at 85° C. for 16 hours. The reaction mixture was concentrated to afford a crude product. The crude product was purified by silica gel chromatography (PE/EtOAc=20/1˜1/1) to give (S)-3-(5-methoxy-1-oxoisoindolin-2-yl)piperidine-2,6-dione (1.00 g, 63.51% yield) as a white solid. ¹H NMR (400 MHz, DMSO_d₆) δ 10.97 (s, 1H), 7.63 (d, J=8.4 Hz, 1H), 7.17 (d, J=2.0 Hz, 1H), 7.05 (dd, J=8.4, 2.0 Hz, 1H), 5.07 (dd, J=13.2, 5.2 Hz, 1H), 4.33 (dd, J=51.2, 17.2 Hz, 2H), 3.84 (s, 3H), 2.97-2.85 (m, 1H), 2.59 (d, J=17.6 Hz, 1H), 2.44-2.32 (m, 1H), 2.03-1.93 (m, 1H).
Step 5: A solution of (S)-3-(5-methoxy-1-oxoisoindolin-2-yl)piperidine-2,6-dione (2.00 g, 7.3 mol) and BBr₃ (18.29 g, 73.00 mmol) in DCM (20 mL) was stirred at 85° C. for 12 hours. The reaction mixture was concentrated to give (S)-3-(5-hydroxy-1-oxoisoindolin-2-yl)piperidine-2,6-dione (1.50 g, 79.0% yield) as a yellow solid. ESI-MS: m/z=261.1 [M+1]⁺.
Step 6: A solution of (S)-3-(5-hydroxy-1-oxoisoindolin-2-yl)piperidine-2,6-dione (3.50 g, 13.40 mmol), 1-Bromo-2-chloroethane (2.31 g, 16.00 mmol), and K₃PO₄ (4.27 g, 20.10 mmol) in DMSO (50 mL) was stirred at 60° C. for 3 hours. The reaction mixture was poured into water (50 mL) and extracted with EtOAc (100 mL×3). The combined organic layers were washed with brine, dried over Na₂SO₄, and concentrated to afford a crude product. The residue was purified by prep-HPLC and lyophilized to give (S)-3-(5-(2-chloroethoxy)-1-oxoisoindolin-2-yl)piperidine-2,6-dione (0.50 g, 11.52% yield) as an off-white solid. ¹H NMR (400 MHz, DMSO-d₆) δ 10.98 (s, 1H), 7.64 (d, J=8.4 Hz, 1H), 7.21 (d, J=2.0 Hz, 1H), 7.09 (dd, J=8.4, 2.0 Hz, 1H), 5.08 (dd, J=13.2, 5.2 Hz, 1H), 4.45-4.32 (m, 3H), 4.27 (d, J=17.2 Hz, 1H), 4.03-3.95 (m, 2H), 2.96-2.84 (m, 1H), 2.59 (d, J=17.2 Hz, 1H), 2.41-2.32 (m, 1H), 2.03-1.93 (m, 1H). ESI-MS: m/z=323.1 [M+1]⁺.

### Method 45

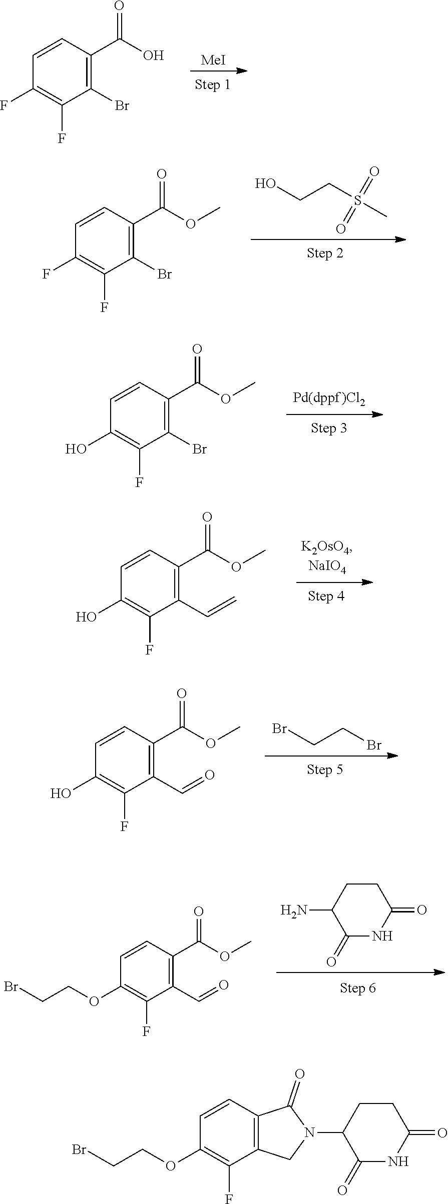

Step 1: A solution of 2-bromo-3,4-difluorobenzoic acid (20.0 g, 84.7 mmol), Iodomethane (14.43 g, 101.64 mmol), and K₂CO₃ (42.14 g, 304.92 mmol) in DMF (200 mL) was stirred at 25° C. for 16 hours. The reaction mixture was poured into water (1 L) and extracted with EtOAc (350 mL×3). The combined organic layers were washed with brine, dried over Na₂SO₄, and concentrated to afford a crude product. The crude product was purified by silica gel chromatography (PE/EtOAc=200/1˜10/1) to give methyl 2-bromo-3,4-difluorobenzoate (17.1 g, 80.7% yield) as a white solid. ¹H NMR (400 MHz, DMSO_d₆) δ 7.77-7.69 (m, 1H), 7.67-7.58 (m, 1H), 3.87 (s, 3H).
Step 2: To a stirred solution of methyl 2-bromo-3,4-difluorobenzoate (17.1 g, 68.4 mmol) in DMF (180 mL) was added NaH (3.2 g, 82.1 mmol) at 0° C. The mixture was stirred at 0° C. for 30 minutes. Then 2-(methylsulfonyl) ethanol (8.5 g, 68.4 mmol) in DMF (80 mL) was slowly added dropwise. The reaction was stirred 0° C. for 2 hours. The reaction mixture was poured into water and extracted with EtOAc (600 mL×3). The combined organic layers were washed with brine, dried over Na₂SO₄, and concentrated to afford a crude product. The crude product was purified by silica gel chromatography (PE/EtOAc=100/1˜3/1) to give methyl 3-amino-2-bromo-4-hydroxybenzoate (9.20 g, 54.7% yield) as a white solid. ¹H NMR (400 MHz, DMSO_d₆) δ 11.21 (s, 1H), 7.62-7.55 (m, 1H), 7.03 (t, J=8.6 Hz, 1H), 3.81 (s, 3H).
Step 3: To a solution of methyl 3-amino-2-bromo-4-hydroxybenzoate (9.20 g, 37.6 mmol) in DMF (100 mL) was added Cs₂CO₃ (36.80 g, 112.8 mmol), Pd(dppf)Cl₂ (920 mg), and Potassium vinyltrifluor (5.10 g, 32.0 mmol). The reaction mixture was stirred at 80° C. for 16 hours under N₂. The reaction mixture was poured into water (500 mL) and extracted with EtOAc (250 mL×3). The combined organic layers were washed with brine, dried over Na₂SO₄, and concentrated to afford a crude product. The crude product was purified by silica gel chromatography (PE/EtOAc=50/1˜2/1) to give methyl 3-fluoro-4-hydroxy-2-vinylbenzoate (3.6 g, 49.7% yield) as a white solid. ¹H NMR (400 MHz, DMSO_d₆) δ 10.74 (s, 1H), 7.62-7.51 (m, 1H), 7.10-6.89 (m, 2H), 5.67-5.54 (m, 2H), 3.77 (s, 3H).
Step 4: To a stirred solution of methyl 3-fluoro-4-hydroxy-2-vinylbenzoate (3.60 g, 18.18 mmol) in THF (40 mL) was added K₂OsO₄ 2H₂O (56.04 mg, 0.18 mmol) at 0° C. The mixture was stirred at 0° C. for 30 minutes. Then NaIO₄ (11.70 g, 54.54 mmol) in H₂O (20 mL) was slowly added dropwise. The reaction was stirred 0° C. for 6 hours. The reaction mixture was poured into water (100 mL) and extracted with EtOAc (50 mL×3). The combined organic layers were washed with brine, dried over Na₂SO₄, and concentrated to afford a crude product. The crude product was purified by silica gel chromatography (PE/EtOAc=100/1˜3/1) to give methyl 3-fluoro-2-formyl-4-hydroxybenzoate (1.50 g, 49.5% yield) as a white solid. ¹H NMR (400 MHz, DMSO_d₆) δ 11.21 (s, 1H), 10.27 (s, 1H), 7.64 (d, J=8.6 Hz, 1H), 7.19 (t, J=8.4 Hz, 1H), 3.83 (s, 3H).
Step 5: A solution of methyl 3-fluoro-2-formyl-4-hydroxybenzoate (1.50 g, 7.57 mmol), 1,2-dibromoethane (1.40 g, 4.74 mmol), and DIEA (2.90 g, 22.71 mmol) in ACN (15 mL) was stirred at 80° C. for 16 hours. The reaction mixture was poured into water (60 mL) and extracted with EtOAc (30 mL×3). The combined organic layers were washed with brine, dried over Na₂SO₄, and concentrated to afford a crude product. The crude product was purified by silica gel chromatography (PE/EtOAc=100/1˜1/1) to give methyl 4-(2-bromoethoxy)-3-fluoro-2-formylbenzoate (1.10 g, 47.6% yield) as a white solid. ¹H NMR (400 MHz, DMSO_d₆) δ 10.28 (s, 1H), 7.75 (d, J=8.6 Hz, 1H), 7.49 (t, J=8.4 Hz, 1H), 4.57-4.49 (m, 2H), 3.88-3.83 (m, 5H).
Step 6: A solution of methyl 4-(2-bromoethoxy)-3-fluoro-2-formylbenzoate (1.10 g, 3.62 mmol), 3-aminopiperidine-2,6-dione hydrochloride (593.7 mg, 3.62 mmol), and DIEA (1.50 g, 10.86 mmol) in DCE (20 mL) was stirred at 40° C. for 30 minutes. The reaction mixture was poured into water (60 mL) and extracted with EtOAc (30 mL×3). The combined organic layers were washed with brine, dried over Na₂SO₄, and concentrated to afford a crude product. The crude product was purified by prep-TLC (EtOAc) to give 3-(5-(2-bromoethoxy)-4-fluoro-1-oxoisoindolin-2-yl) piperidine-2,6-dione (250 mg, 19.8% yield) as a blue solid. ¹H NMR (400 MHz, DMSO_d₆) δ 10.99 (s, 1H), 7.54 (d, J=8.4 Hz, 1H), 7.37 (t, J=7.6 Hz, 1H), 5.13-5.04 (m, 1H), 4.61-4.46 (m, 3H), 4.36 (d, J=17.0 Hz, 1H), 3.86 (t, J=5.4 Hz, 2H), 2.98-2.85 (m, 1H), 2.60-2.45 (m, 1H), 2.45-2.31 (m, 2H), 2.05-1.92 (m, 1H). ESI-MS: m/z=387.0[M+1]⁺.

### Method 46

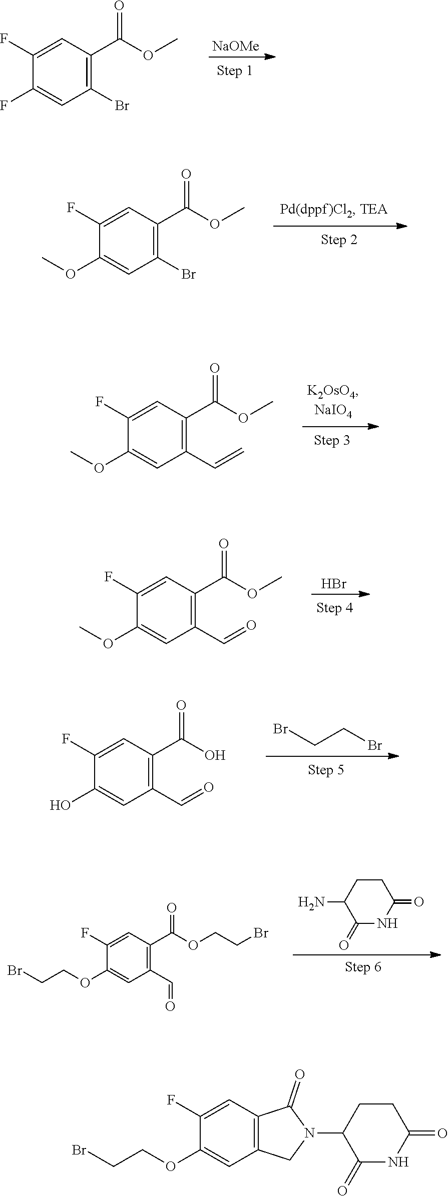

Step 1: To a stirred solution of methyl 2-bromo-4,5-difluorobenzoate (10.00 g, 39.80 mmol) in THF (100 mL) was added MeONa (8.60 g, 47.70 mmol) portion wise at 0° C. The mixture was stirred at 25° C. for 1 hour. The reaction mixture was concentrated to afford a crude product. The crude product was purified by silica gel chromatography (PE/EtOAc=20/1˜5/1) to give methyl 2-bromo-5-fluoro-4-methoxybenzoate (6.00 g, 57.3% yield) as a yellow oil. ¹H NMR (400 MHz, DMSO_d₆) δ 7.70 (d, J=12.0 Hz, 1H), 7.51 (d, J=8.0 Hz, 1H), 3.93 (s, 3H).
Step 2: A solution of methyl 2-bromo-5-fluoro-4-methoxybenzoate (10.00 g, 380.0 mmol), potassium trifluoro(vinyl)borate (7.64 g, 56.9 mmol), TEA (24.76 g, 76.0 mmol), and Pd(dppf)Cl₂ (1.39 g, 1.9 mmol) in DMF (100 mL) was stirred at 90° C. for 3 hours. The reaction mixture was poured into water (500 mL) and extracted with EtOAc (50 mL×3). The combined organic layers were washed with brine, dried over Na₂SO₄, and concentrated to afford a crude product. The crude product was purified by silica gel chromatography (PE/EtOAc=20/1˜5/1) to give methyl 5-fluoro-4-methoxy-2-vinylbenzoate (6.00 g, 75.1% yield) as a white solid. ESI-MS: m/z=211.1 [M+1]⁺.
Step 3: To a stirred solution of methyl 5-fluoro-4-methoxy-2-vinylbenzoate (5.00 g, 0.0238 mol) in THF/H₂O (3/1, 80 mL) was added Potassium osmate(VI) dihydrate (0.15 g, 0.4 mmol) dropwise at 0° C. The mixture was stirred at 0° C. for 15 minutes. Then NaIO₄ (15.27 g, 71.4 mmol) was added. The reaction was stirred 25° C. for 12 hours. The mixture was filtered. The filtrate was concentrated, poured into water (100 mL), and extracted with EtOAc (50 mL×3). The combined organic layers were washed with brine, dried over Na₂SO₄, and concentrated to afford a crude product. The crude product was purified by silica gel chromatography (PE/EtOAc=20/1˜5/1) to give methyl 5-fluoro-2-formyl-4-methoxybenzoate (3.00 g, 59.4% yield) as an off-white solid. ¹H NMR (400 MHz, DMSO_d₆) δ 10.43 (s, 1H), 7.78 (d, J=11.6 Hz, 1H), 7.55 (d, J=8.4 Hz, 1H), 3.98 (s, 3H), 3.88 (s, 3H).
Step 4: A solution of methyl 5-fluoro-2-formyl-4-methoxybenzoate (3.50 g, 16.5 mmol) in 48% HBr (50 mL) was stirred at 100° C. for 48 hours. The reaction mixture was concentrated to afford a crude product. The crude product was purified by silica gel chromatography (PE/EtOAc=20/1˜1/1) to give 5-fluoro-2-formyl-4-hydroxybenzoic acid (1.30 g, 42.8% yield) as a white solid.
Step 5: A solution of 5-fluoro-2-formyl-4-hydroxybenzoic acid (3.00 g, 16.30 mol), 1,2-dibromoethane (21.43 g, 114.10 mmol), and K₂CO₃ (6.76 g, 48.90 mmol) in DMSO (20 mL) was stirred under nitrogen at 25° C. for 3 hours. The reaction mixture was poured into water (50 mL) and extracted with EtOAc (20 mL×3). The combined organic layers were washed with brine, dried over Na₂SO₄, and concentrated to afford a crude product. The crude product was purified by prep-TLC (PE/EtOAc=5/1) to give 2-bromoethyl 4-(2-bromoethoxy)-5-fluoro-2-formylbenzoate (2.70 g, 42.8% yield) as a yellow oil. ESI-MS: m/z=398.8 [M+1]⁺.
Step 6: To a stirred solution of 2-bromoethyl 4-(2-bromoethoxy)-5-fluoro-2-formylbenzoate (2.50 g, 6.3 mmol) in DCE (25 mL) was added 2-bromoethyl 4-(2-bromoethoxy)-5-fluoro-2-formylbenzoate (0.97 g, 7.5 mmol), DIEA (1.95 g, 15.1 mmol), and AcOH (1 mL) at 40° C. The mixture was stirred at 40° C. for 12 hours. Then NaBH₃CN (1.19 g, 0.0189 mol) was added. The reaction was stirred 40° C. for 4 hours. The reaction mixture was concentrated to afford a crude product. The crude product was purified by silica gel chromatography (PE/EtOAc=20/1˜1/1) to give 3-(5-(2-bromoethoxy)-6-fluoro-1-oxoisoindolin-2-yl)piperidine-2,6-dione (0.50 g, 20.7% yield) as a blue solid. ¹H NMR (400 MHz, DMSO-d₆) δ 10.99 (s, 1H), 7.56 (d, J=10.0 Hz, 1H), 7.46 (d, J=7.2 Hz, 1H), 5.09 (dd, J=13.2, 5.2 Hz, 1H), 4.54-4.45 (m, 2H), 4.33 (dd, J=48.0, 17.2 Hz, 2H), 3.88 (t, J=5.2 Hz, 2H), 2.95-2.83 (m, 1H), 2.59 (d, J=17.6 Hz, 1H), 2.45-2.34 (m, 1H), 2.04-1.94 (in, 1H). ESI-MS: m/z=385.1, 387.1 [M+1]⁺.

The following compounds (9) 2-1 to 2-187 shown in Table 2 were prepared by methods similar to those described for the preparation of compound 2-43, 2-120, or more using the appropriate Boc-amine (2) and the ether intermediate (10).

TABLE 2

| Compd | | |
|---|---|---|
| | 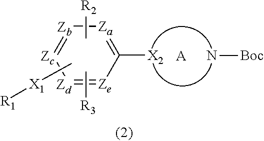 | 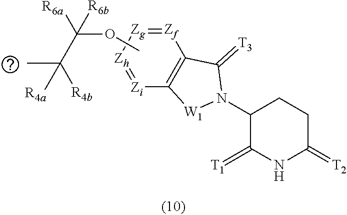 |
| 2-1 | 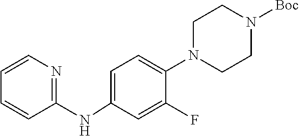 [SMILES: CC(C)(C)OC(=O)N1CCN(c2ccc(Nc3ccccn3)cc2F)CC1] | 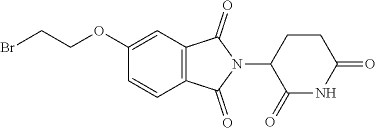 [SMILES: O=C1CCC(N2C(=O)c3ccc(OCCBr)cc3C2=O)C(=O)N1] |
| 2-2 | 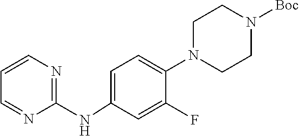 [SMILES: CC(C)(C)OC(=O)N1CCN(c2ccc(Nc3ncccn3)cc2F)CC1] | 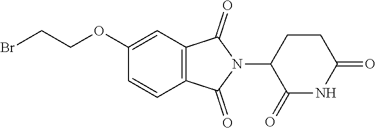 [SMILES: O=C1CCC(N2C(=O)c3ccc(OCCBr)cc3C2=O)C(=O)N1] |
| 2-3 | 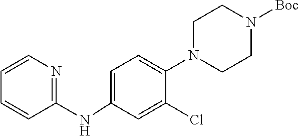 [SMILES: CC(C)(C)OC(=O)N1CCN(c2ccc(Nc3ccccn3)cc2Cl)CC1] | 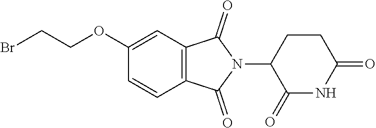 [SMILES: O=C1CCC(N2C(=O)c3ccc(OCCBr)cc3C2=O)C(=O)N1] |
| 2-4 | 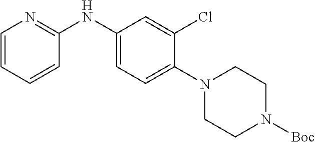 [SMILES: CC(C)(C)OC(=O)N1CCN(c2ccc(Nc3ccccn3)cc2Cl)CC1] | 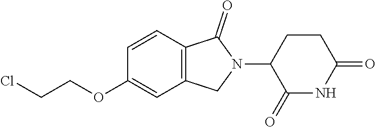 [SMILES: O=C1CCC(N2Cc3cc(OCCCl)ccc3C2=O)C(=O)N1] |
| 2-5 | 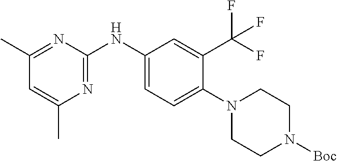 [SMILES: Cc1cc(C)nc(Nc2ccc(N3CCN(C(=O)OC(C)(C)C)CC3)c(C(F)(F)F)c2)n1] | 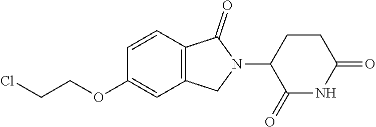 [SMILES: O=C1CCC(N2Cc3cc(OCCCl)ccc3C2=O)C(=O)N1] |
| 2-6 | 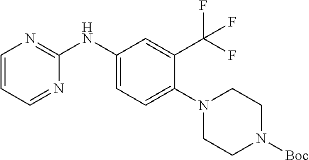 [SMILES: CC(C)(C)OC(=O)N1CCN(c2ccc(Nc3ncccn3)cc2C(F)(F)F)CC1] | 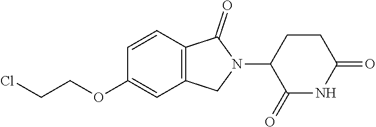 [SMILES: O=C1CCC(N2Cc3cc(OCCCl)ccc3C2=O)C(=O)N1] |
| 2-7 | 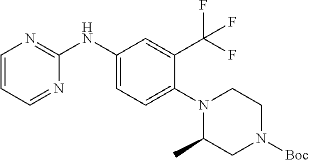 [SMILES: C[C@@H]1CN(C(=O)OC(C)(C)C)CCN1c1ccc(Nc2ncccn2)cc1C(F)(F)F] | 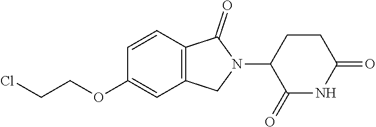 [SMILES: O=C1CCC(N2Cc3cc(OCCCl)ccc3C2=O)C(=O)N1] |
| 2-8 | 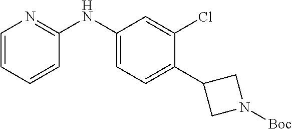 [SMILES: CC(C)(C)OC(=O)N1CC(c2ccc(Nc3ccccn3)cc2Cl)C1] | 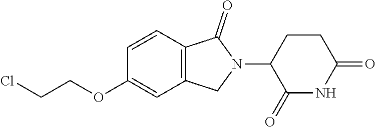 [SMILES: O=C1CCC(N2Cc3cc(OCCCl)ccc3C2=O)C(=O)N1] |
| 2-9 | 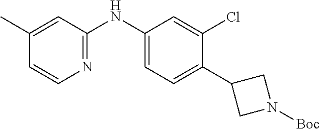 [SMILES: Cc1ccnc(Nc2ccc(C3CN(C(=O)OC(C)(C)C)C3)c(Cl)c2)c1] | 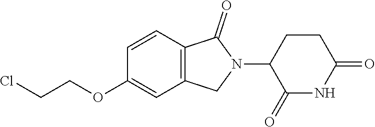 [SMILES: O=C1CCC(N2Cc3cc(OCCCl)ccc3C2=O)C(=O)N1] |
| 2-10 | 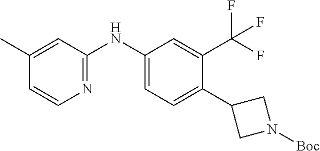 [SMILES: Cc1ccnc(Nc2ccc(C3CN(C(=O)OC(C)(C)C)C3)c(C(F)(F)F)c2)c1] | 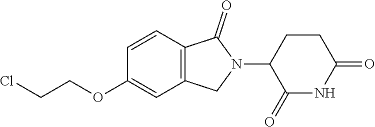 [SMILES: O=C1CCC(N2Cc3cc(OCCCl)ccc3C2=O)C(=O)N1] |
| 2-11 | 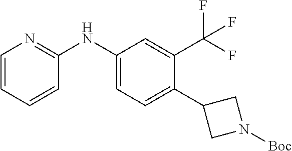 [SMILES: CC(C)(C)OC(=O)N1CC(c2ccc(Nc3ccccn3)cc2C(F)(F)F)C1] | 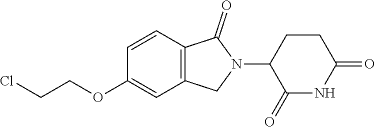 [SMILES: O=C1CCC(N2Cc3cc(OCCCl)ccc3C2=O)C(=O)N1] |
| 2-12 | 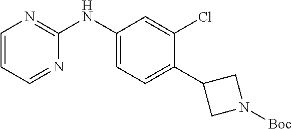 [SMILES: CC(C)(C)OC(=O)N1CC(c2ccc(Nc3ncccn3)cc2Cl)C1] | 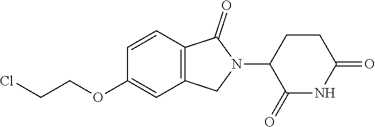 [SMILES: O=C1CCC(N2Cc3cc(OCCCl)ccc3C2=O)C(=O)N1] |
| 2-13 | 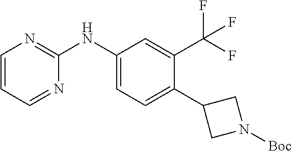 [SMILES: CC(C)(C)OC(=O)N1CC(c2ccc(Nc3ncccn3)cc2C(F)(F)F)C1] | 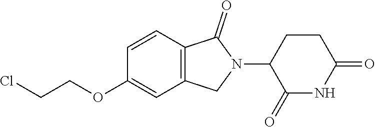 [SMILES: O=C1CCC(N2Cc3cc(OCCCl)ccc3C2=O)C(=O)N1] |
| 2-14 | 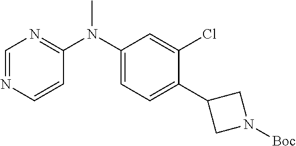 [SMILES: CN(c1ccc(C2CN(C(=O)OC(C)(C)C)C2)c(Cl)c1)c1ccncn1] | 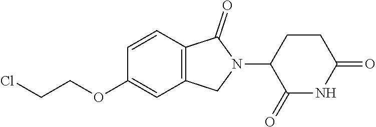 [SMILES: O=C1CCC(N2Cc3cc(OCCCl)ccc3C2=O)C(=O)N1] |
| 2-15 | 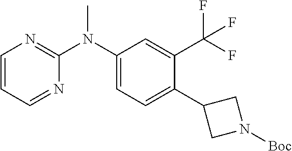 [SMILES: CN(c1ccc(C2CN(C(=O)OC(C)(C)C)C2)c(C(F)(F)F)c1)c1ncccn1] | 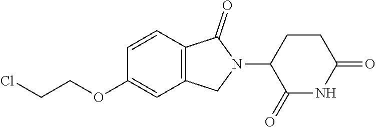 [SMILES: O=C1CCC(N2Cc3cc(OCCCl)ccc3C2=O)C(=O)N1] |
| 2-16 | 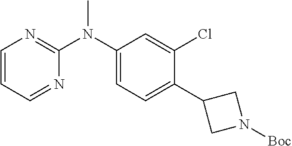 [SMILES: CN(c1ccc(C2CN(C(=O)OC(C)(C)C)C2)c(Cl)c1)c1ncccn1] | 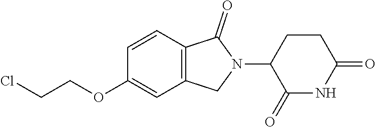 [SMILES: O=C1CCC(N2Cc3cc(OCCCl)ccc3C2=O)C(=O)N1] |
| 2-17 | 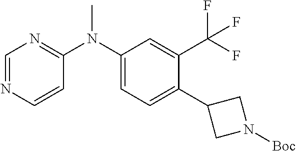 [SMILES: CN(c1ccc(C2CN(C(=O)OC(C)(C)C)C2)c(C(F)(F)F)c1)c1ccncn1] | 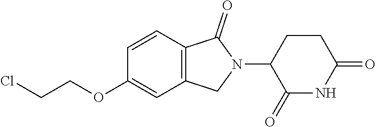 [SMILES: O=C1CCC(N2Cc3cc(OCCCl)ccc3C2=O)C(=O)N1] |
| 2-18 | 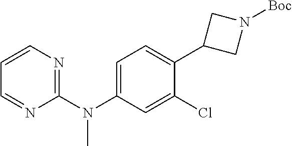 [SMILES: CN(c1ccc(C2CN(C(=O)OC(C)(C)C)C2)c(Cl)c1)c1ncccn1] | 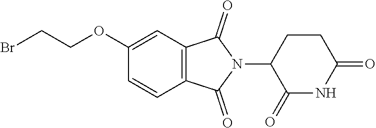 [SMILES: O=C1CCC(N2C(=O)c3ccc(OCCBr)cc3C2=O)C(=O)N1] |
| 2-19 | 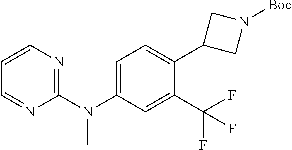 [SMILES: CN(c1ccc(C2CN(C(=O)OC(C)(C)C)C2)c(C(F)(F)F)c1)c1ncccn1] | 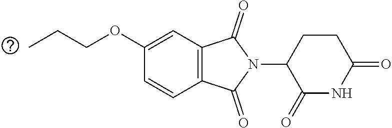 [SMILES: CCCOc1ccc2c(c1)C(=O)N(C1CCC(=O)NC1=O)C2=O] |
| 2-20 | 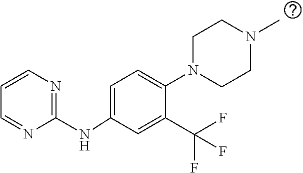 [SMILES: CN1CCN(c2ccc(Nc3ncccn3)cc2C(F)(F)F)CC1] | 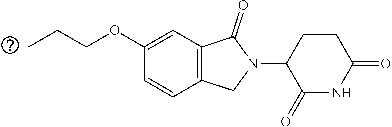 [SMILES: CCCOc1ccc2c(c1)C(=O)N(C1CCC(=O)NC1=O)C2] |
| 2-21 | 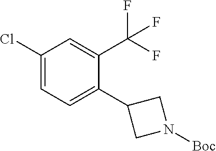 [SMILES: CC(C)(C)OC(=O)N1CC(c2ccc(Cl)cc2C(F)(F)F)C1] | 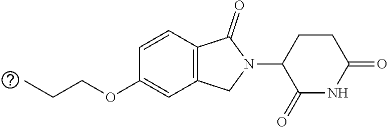 [SMILES: CCCOc1ccc2c(c1)CN(C1CCC(=O)NC1=O)C2=O] |
| 2-22 | 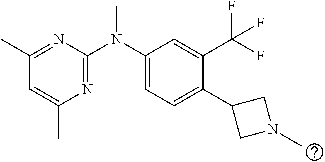 | 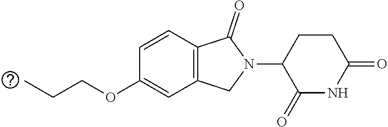 [SMILES: CCCOc1ccc2c(c1)CN(C1CCC(=O)NC1=O)C2=O] |
| 2-23 | 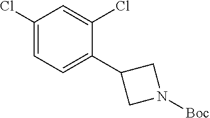 [SMILES: CC(C)(C)OC(=O)N1CC(c2ccc(Cl)cc2Cl)C1] | 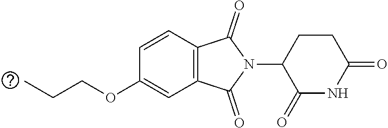 [SMILES: CCCOc1ccc2c(c1)C(=O)N(C1CCC(=O)NC1=O)C2=O] |
| 2-24 | 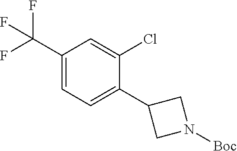 [SMILES: CC(C)(C)OC(=O)N1CC(c2ccc(C(F)(F)F)cc2Cl)C1] | 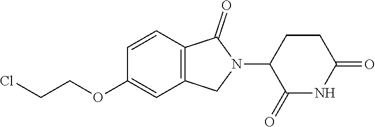 [SMILES: O=C1CCC(N2Cc3cc(OCCCl)ccc3C2=O)C(=O)N1] |
| 2-25 | 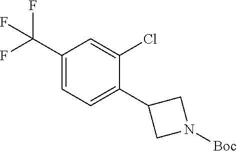 [SMILES: CC(C)(C)OC(=O)N1CC(c2ccc(C(F)(F)F)cc2Cl)C1] | 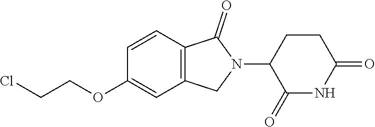 [SMILES: O=C1CCC(N2Cc3cc(OCCCl)ccc3C2=O)C(=O)N1] |
| 2-26 | 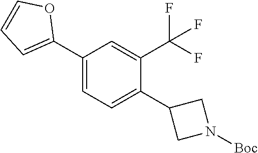 [SMILES: CC(C)(C)OC(=O)N1CC(c2ccc(-c3ccco3)cc2C(F)(F)F)C1] | 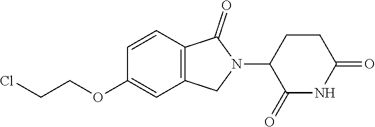 [SMILES: O=C1CCC(N2Cc3cc(OCCCl)ccc3C2=O)C(=O)N1] |
| 2-27 | 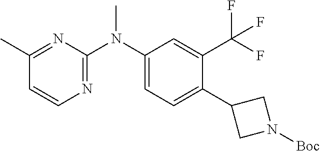 [SMILES: Cc1ccnc(N(C)c2ccc(C3CN(C(=O)OC(C)(C)C)C3)c(C(F)(F)F)c2)n1] | 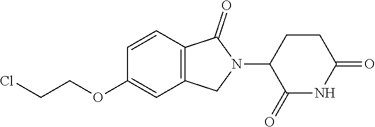 [SMILES: O=C1CCC(N2Cc3cc(OCCCl)ccc3C2=O)C(=O)N1] |
| 2-28 | 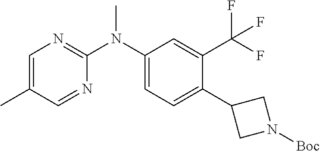 [SMILES: Cc1cnc(N(C)c2ccc(C3CN(C(=O)OC(C)(C)C)C3)c(C(F)(F)F)c2)nc1] | 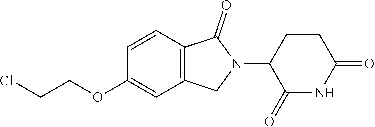 [SMILES: O=C1CCC(N2Cc3cc(OCCCl)ccc3C2=O)C(=O)N1] |
| 2-29 | 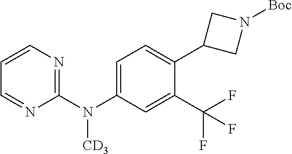 [SMILES: [2H]C([2H])([2H])N(c1ccc(C2CN(C(=O)OC(C)(C)C)C2)c(C(F)(F)F)c1)c1ncccn1] | 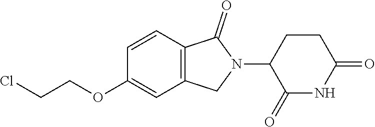 [SMILES: O=C1CCC(N2Cc3cc(OCCCl)ccc3C2=O)C(=O)N1] |
| 2-30 | 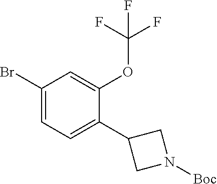 [SMILES: CC(C)(C)OC(=O)N1CC(c2ccc(Br)cc2OC(F)(F)F)C1] | 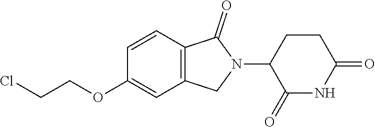 [SMILES: O=C1CCC(N2Cc3cc(OCCCl)ccc3C2=O)C(=O)N1] |
| 2-31 | 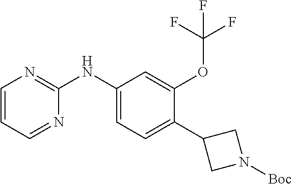 [SMILES: CC(C)(C)OC(=O)N1CC(c2ccc(Nc3ncccn3)cc2OC(F)(F)F)C1] | 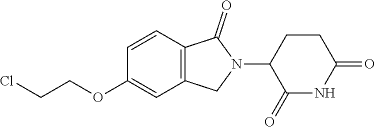 [SMILES: O=C1CCC(N2Cc3cc(OCCCl)ccc3C2=O)C(=O)N1] |
| 2-32 | 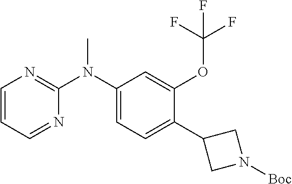 [SMILES: CN(c1ccc(C2CN(C(=O)OC(C)(C)C)C2)c(OC(F)(F)F)c1)c1ncccn1] | 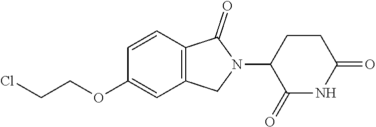 [SMILES: O=C1CCC(N2Cc3cc(OCCCl)ccc3C2=O)C(=O)N1] |
| 2-33 | 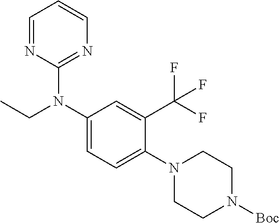 [SMILES: CCN(c1ccc(N2CCN(C(=O)OC(C)(C)C)CC2)c(C(F)(F)F)c1)c1ncccn1] | 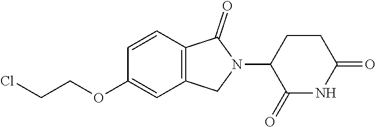 [SMILES: O=C1CCC(N2Cc3cc(OCCCl)ccc3C2=O)C(=O)N1] |
| 2-34 | 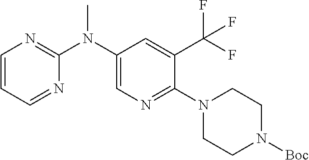 [SMILES: CN(c1cnc(N2CCN(C(=O)OC(C)(C)C)CC2)c(C(F)(F)F)c1)c1ncccn1] | 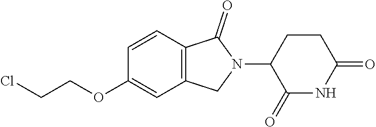 [SMILES: O=C1CCC(N2Cc3cc(OCCCl)ccc3C2=O)C(=O)N1] |
| 2-35 | 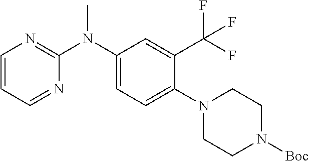 [SMILES: CN(c1ccc(N2CCN(C(=O)OC(C)(C)C)CC2)c(C(F)(F)F)c1)c1ncccn1] | 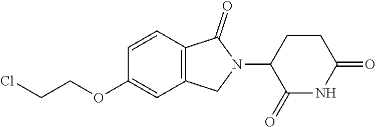 [SMILES: O=C1CCC(N2Cc3cc(OCCCl)ccc3C2=O)C(=O)N1] |
| 2-36 | 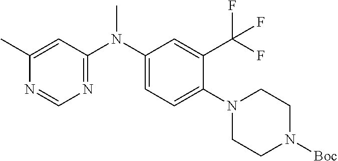 [SMILES: Cc1cc(N(C)c2ccc(N3CCN(C(=O)OC(C)(C)C)CC3)c(C(F)(F)F)c2)ncn1] | 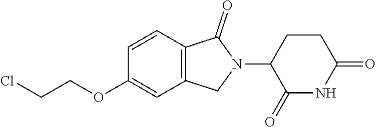 [SMILES: O=C1CCC(N2Cc3cc(OCCCl)ccc3C2=O)C(=O)N1] |
| 2-37 | 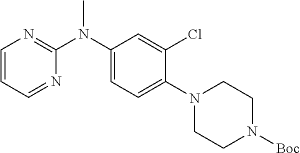 [SMILES: CN(c1ccc(N2CCN(C(=O)OC(C)(C)C)CC2)c(Cl)c1)c1ncccn1] | 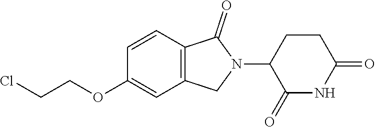 [SMILES: O=C1CCC(N2Cc3cc(OCCCl)ccc3C2=O)C(=O)N1] |
| 2-38 | 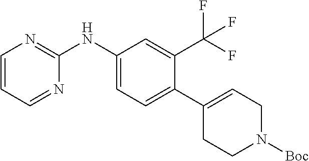 [SMILES: CC(C)(C)OC(=O)N1CC=C(c2ccc(Nc3ncccn3)cc2C(F)(F)F)CC1] | 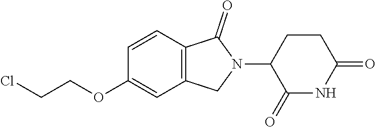 [SMILES: O=C1CCC(N2Cc3cc(OCCCl)ccc3C2=O)C(=O)N1] |
| 2-39 | 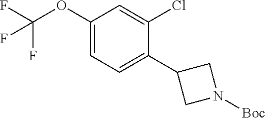 [SMILES: CC(C)(C)OC(=O)N1CC(c2ccc(OC(F)(F)F)cc2Cl)C1] | 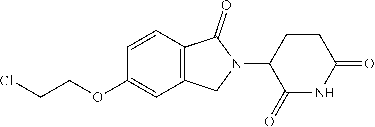 [SMILES: O=C1CCC(N2Cc3cc(OCCCl)ccc3C2=O)C(=O)N1] |
| 2-40 | 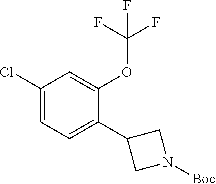 [SMILES: CC(C)(C)OC(=O)N1CC(c2ccc(Cl)cc2OC(F)(F)F)C1] | 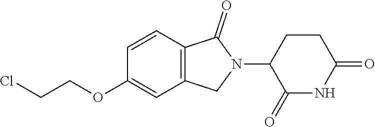 [SMILES: O=C1CCC(N2Cc3cc(OCCCl)ccc3C2=O)C(=O)N1] |
| 2-41 | 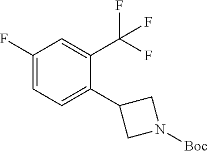 [SMILES: CC(C)(C)OC(=O)N1CC(c2ccc(F)cc2C(F)(F)F)C1] | 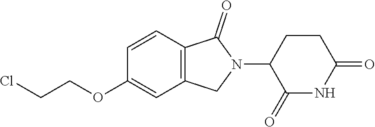 [SMILES: O=C1CCC(N2Cc3cc(OCCCl)ccc3C2=O)C(=O)N1] |
| 2-42 | 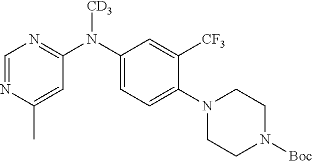 [SMILES: [2H]C([2H])([2H])N(c1ccc(N2CCN(C(=O)OC(C)(C)C)CC2)c(C(F)(F)F)c1)c1cc(C)ncn1] | 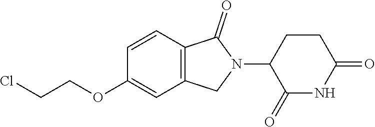 [SMILES: O=C1CCC(N2Cc3cc(OCCCl)ccc3C2=O)C(=O)N1] |
| 2-43 | 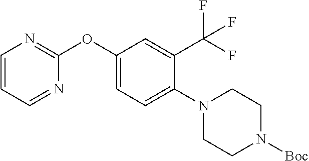 [SMILES: CC(C)(C)OC(=O)N1CCN(c2ccc(Oc3ncccn3)cc2C(F)(F)F)CC1] | 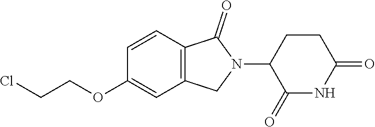 [SMILES: O=C1CCC(N2Cc3cc(OCCCl)ccc3C2=O)C(=O)N1] |
| 2-44 | 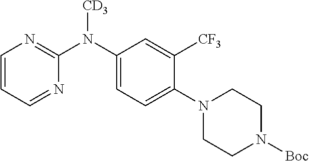 [SMILES: [2H]C([2H])([2H])N(c1ccc(N2CCN(C(=O)OC(C)(C)C)CC2)c(C(F)(F)F)c1)c1ncccn1] | 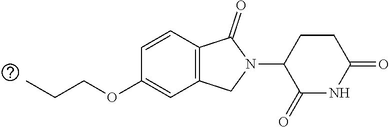 [SMILES: CCCOc1ccc2c(c1)CN(C1CCC(=O)NC1=O)C2=O] |
| 2-45 | 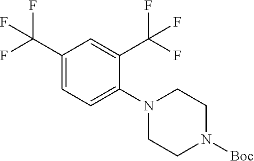 [SMILES: CC(C)(C)OC(=O)N1CCN(c2ccc(C(F)(F)F)cc2C(F)(F)F)CC1] | 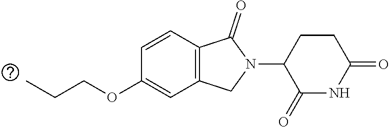 [SMILES: CCCOc1ccc2c(c1)CN(C1CCC(=O)NC1=O)C2=O] |
| 2-46 | 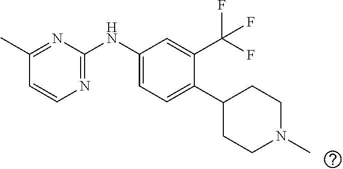 | 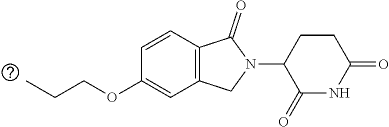 [SMILES: CCCOc1ccc2c(c1)CN(C1CCC(=O)NC1=O)C2=O] |
| 2-47 | 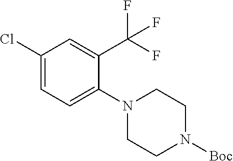 [SMILES: CC(C)(C)OC(=O)N1CCN(c2ccc(Cl)cc2C(F)(F)F)CC1] | 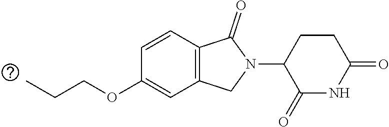 [SMILES: CCCOc1ccc2c(c1)CN(C1CCC(=O)NC1=O)C2=O] |
| 2-48 | 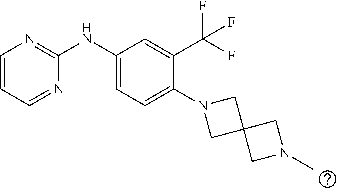 [SMILES: CN1CC2(C1)CN(c1ccc(Nc3ncccn3)cc1C(F)(F)F)C2] | 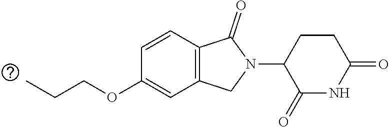 [SMILES: CCCOc1ccc2c(c1)CN(C1CCC(=O)NC1=O)C2=O] |
| 2-49 | 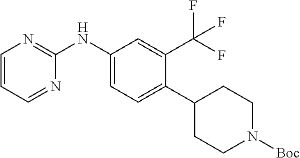 [SMILES: CC(C)(C)OC(=O)N1CCC(c2ccc(Nc3ncccn3)cc2C(F)(F)F)CC1] | 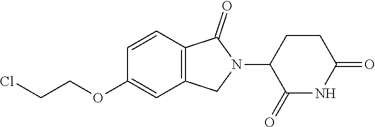 [SMILES: O=C1CCC(N2Cc3cc(OCCCl)ccc3C2=O)C(=O)N1] |
| 2-50 | 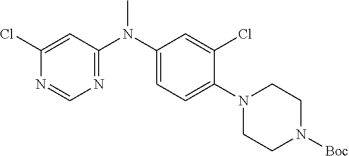 [SMILES: CN(c1ccc(N2CCN(C(=O)OC(C)(C)C)CC2)c(Cl)c1)c1cc(Cl)ncn1] | 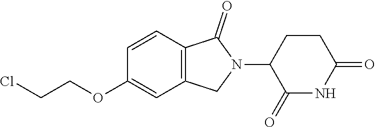 [SMILES: O=C1CCC(N2Cc3cc(OCCCl)ccc3C2=O)C(=O)N1] |
| 2-51 | 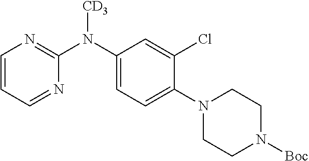 [SMILES: [2H]C([2H])([2H])N(c1ccc(N2CCN(C(=O)OC(C)(C)C)CC2)c(Cl)c1)c1ncccn1] | 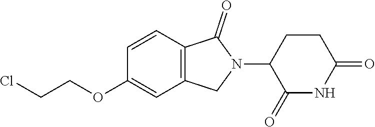 [SMILES: O=C1CCC(N2Cc3cc(OCCCl)ccc3C2=O)C(=O)N1] |
| 2-52 | 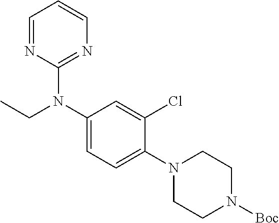 [SMILES: CCN(c1ccc(N2CCN(C(=O)OC(C)(C)C)CC2)c(Cl)c1)c1ncccn1] | 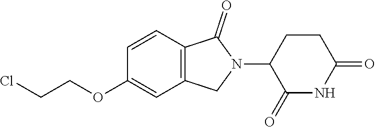 [SMILES: O=C1CCC(N2Cc3cc(OCCCl)ccc3C2=O)C(=O)N1] |
| 2-53 | 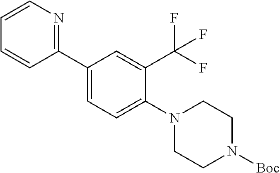 [SMILES: CC(C)(C)OC(=O)N1CCN(c2ccc(-c3ccccn3)cc2C(F)(F)F)CC1] | 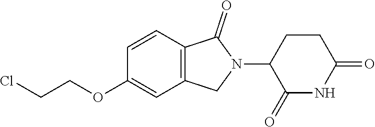 [SMILES: O=C1CCC(N2Cc3cc(OCCCl)ccc3C2=O)C(=O)N1] |
| 2-54 | 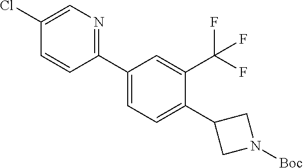 [SMILES: CC(C)(C)OC(=O)N1CC(c2ccc(-c3ccc(Cl)cn3)cc2C(F)(F)F)C1] | 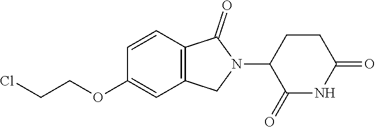 [SMILES: O=C1CCC(N2Cc3cc(OCCCl)ccc3C2=O)C(=O)N1] |
| 2-55 | 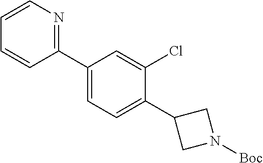 [SMILES: CC(C)(C)OC(=O)N1CC(c2ccc(-c3ccccn3)cc2Cl)C1] | 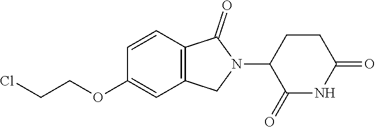 [SMILES: O=C1CCC(N2Cc3cc(OCCCl)ccc3C2=O)C(=O)N1] |
| 2-56 | 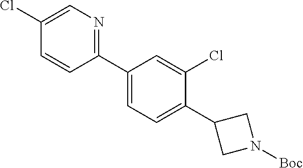 [SMILES: CC(C)(C)OC(=O)N1CC(c2ccc(-c3ccc(Cl)cn3)cc2Cl)C1] | 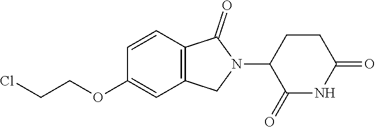 [SMILES: O=C1CCC(N2Cc3cc(OCCCl)ccc3C2=O)C(=O)N1] |
| 2-57 | 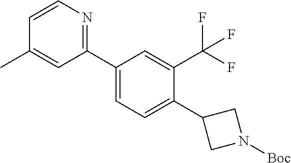 [SMILES: Cc1ccnc(-c2ccc(C3CN(C(=O)OC(C)(C)C)C3)c(C(F)(F)F)c2)c1] | 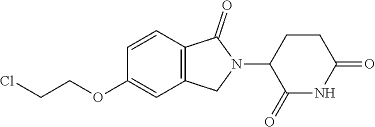 [SMILES: O=C1CCC(N2Cc3cc(OCCCl)ccc3C2=O)C(=O)N1] |
| 2-58 | 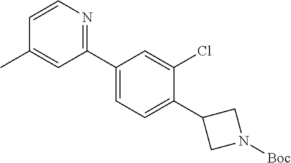 [SMILES: Cc1ccnc(-c2ccc(C3CN(C(=O)OC(C)(C)C)C3)c(Cl)c2)c1] | 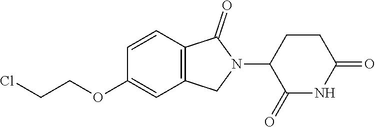 [SMILES: O=C1CCC(N2Cc3cc(OCCCl)ccc3C2=O)C(=O)N1] |
| 2-59 | 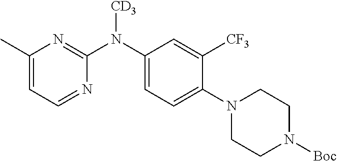 [SMILES: [2H]C([2H])([2H])N(c1ccc(N2CCN(C(=O)OC(C)(C)C)CC2)c(C(F)(F)F)c1)c1nccc(C)n1] | 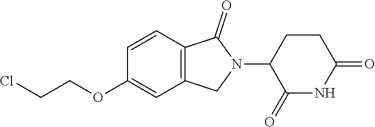 [SMILES: O=C1CCC(N2Cc3cc(OCCCl)ccc3C2=O)C(=O)N1] |
| 2-60 | 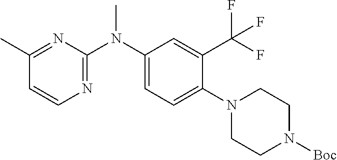 [SMILES: Cc1ccnc(N(C)c2ccc(N3CCN(C(=O)OC(C)(C)C)CC3)c(C(F)(F)F)c2)n1] | 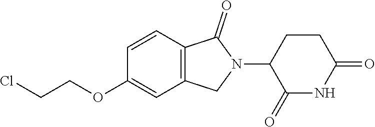 [SMILES: O=C1CCC(N2Cc3cc(OCCCl)ccc3C2=O)C(=O)N1] |
| 2-61 | 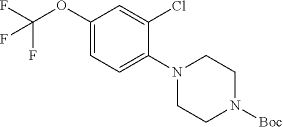 [SMILES: CC(C)(C)OC(=O)N1CCN(c2ccc(OC(F)(F)F)cc2Cl)CC1] | 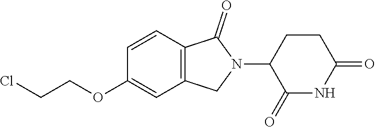 [SMILES: O=C1CCC(N2Cc3cc(OCCCl)ccc3C2=O)C(=O)N1] |
| 2-62 | 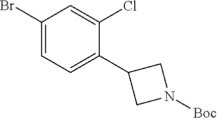 [SMILES: CC(C)(C)OC(=O)N1CC(c2ccc(Br)cc2Cl)C1] | 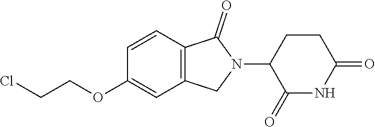 [SMILES: O=C1CCC(N2Cc3cc(OCCCl)ccc3C2=O)C(=O)N1] |
| 2-63 | 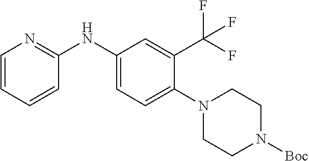 [SMILES: CC(C)(C)OC(=O)N1CCN(c2ccc(Nc3ccccn3)cc2C(F)(F)F)CC1] | 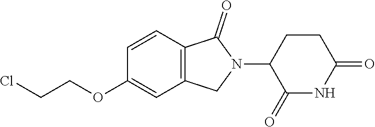 [SMILES: O=C1CCC(N2Cc3cc(OCCCl)ccc3C2=O)C(=O)N1] |
| 2-64 | 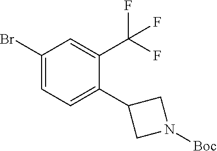 [SMILES: CC(C)(C)OC(=O)N1CC(c2ccc(Br)cc2C(F)(F)F)C1] | 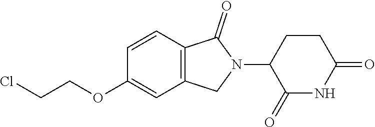 [SMILES: O=C1CCC(N2Cc3cc(OCCCl)ccc3C2=O)C(=O)N1] |
| 2-65 | 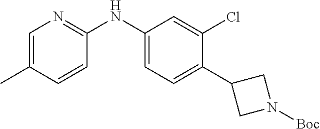 [SMILES: Cc1ccc(Nc2ccc(C3CN(C(=O)OC(C)(C)C)C3)c(Cl)c2)nc1] | 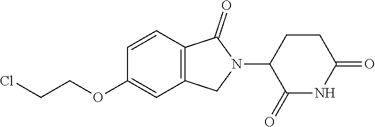 [SMILES: O=C1CCC(N2Cc3cc(OCCCl)ccc3C2=O)C(=O)N1] |
| 2-66 | 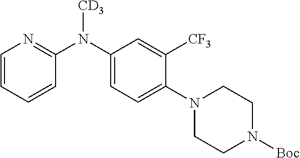 [SMILES: [2H]C([2H])([2H])N(c1ccc(N2CCN(C(=O)OC(C)(C)C)CC2)c(C(F)(F)F)c1)c1ccccn1] | 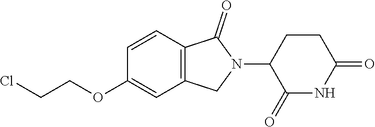 [SMILES: O=C1CCC(N2Cc3cc(OCCCl)ccc3C2=O)C(=O)N1] |
| 2-67 | 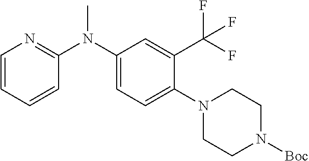 [SMILES: CN(c1ccc(N2CCN(C(=O)OC(C)(C)C)CC2)c(C(F)(F)F)c1)c1ccccn1] | 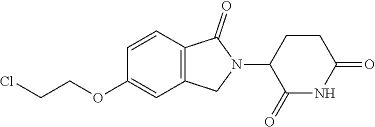 [SMILES: O=C1CCC(N2Cc3cc(OCCCl)ccc3C2=O)C(=O)N1] |
| 2-68 | 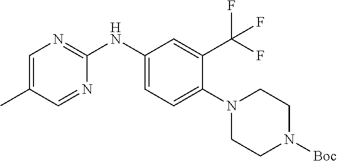 [SMILES: Cc1cnc(Nc2ccc(N3CCN(C(=O)OC(C)(C)C)CC3)c(C(F)(F)F)c2)nc1] | 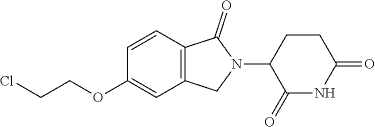 [SMILES: O=C1CCC(N2Cc3cc(OCCCl)ccc3C2=O)C(=O)N1] |
| 2-69 | 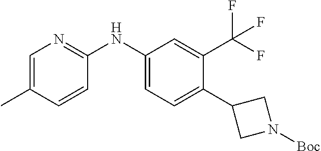 [SMILES: Cc1ccc(Nc2ccc(C3CN(C(=O)OC(C)(C)C)C3)c(C(F)(F)F)c2)nc1] | 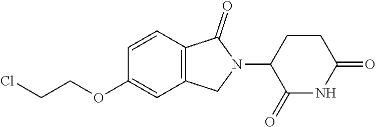 [SMILES: O=C1CCC(N2Cc3cc(OCCCl)ccc3C2=O)C(=O)N1] |
| 2-70 | 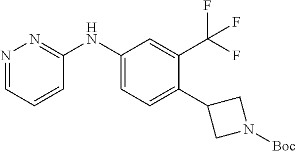 [SMILES: CC(C)(C)OC(=O)N1CC(c2ccc(Nc3cccnn3)cc2C(F)(F)F)C1] | 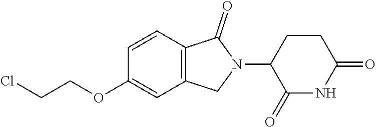 [SMILES: O=C1CCC(N2Cc3cc(OCCCl)ccc3C2=O)C(=O)N1] |
| 2-71 | 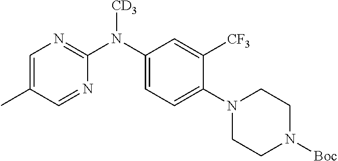 [SMILES: [2H]C([2H])([2H])N(c1ccc(N2CCN(C(=O)OC(C)(C)C)CC2)c(C(F)(F)F)c1)c1ncc(C)cn1] | 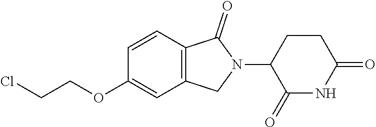 [SMILES: O=C1CCC(N2Cc3cc(OCCCl)ccc3C2=O)C(=O)N1] |
| 2-72 | 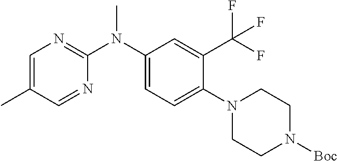 [SMILES: Cc1cnc(N(C)c2ccc(N3CCN(C(=O)OC(C)(C)C)CC3)c(C(F)(F)F)c2)nc1] | 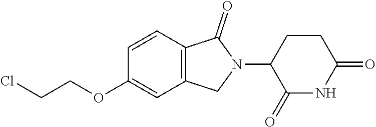 [SMILES: O=C1CCC(N2Cc3cc(OCCCl)ccc3C2=O)C(=O)N1] |
| 2-73 | 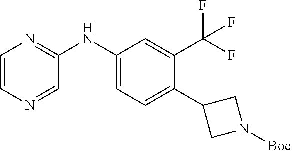 [SMILES: CC(C)(C)OC(=O)N1CC(c2ccc(Nc3cnccn3)cc2C(F)(F)F)C1] | 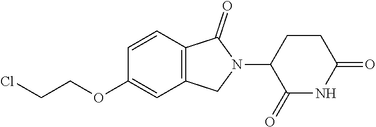 [SMILES: O=C1CCC(N2Cc3cc(OCCCl)ccc3C2=O)C(=O)N1] |
| 2-74 | 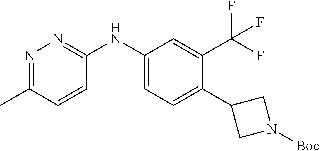 [SMILES: Cc1ccc(Nc2ccc(C3CN(C(=O)OC(C)(C)C)C3)c(C(F)(F)F)c2)nn1] | 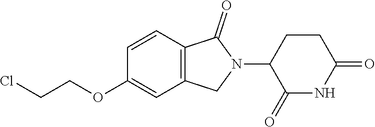 [SMILES: O=C1CCC(N2Cc3cc(OCCCl)ccc3C2=O)C(=O)N1] |
| 2-75 | 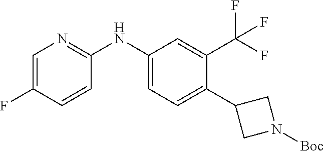 [SMILES: CC(C)(C)OC(=O)N1CC(c2ccc(Nc3ccc(F)cn3)cc2C(F)(F)F)C1] | 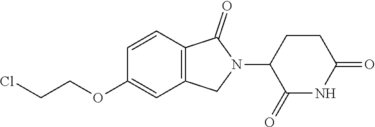 [SMILES: O=C1CCC(N2Cc3cc(OCCCl)ccc3C2=O)C(=O)N1] |
| 2-76 | 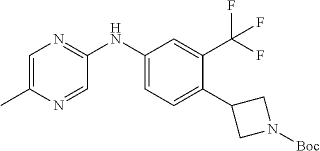 [SMILES: Cc1cnc(Nc2ccc(C3CN(C(=O)OC(C)(C)C)C3)c(C(F)(F)F)c2)cn1] | 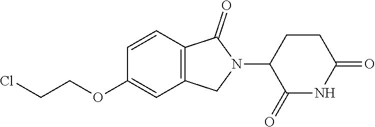 [SMILES: O=C1CCC(N2Cc3cc(OCCCl)ccc3C2=O)C(=O)N1] |
| 2-77 | 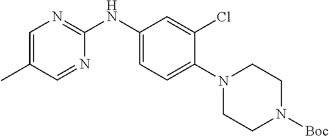 [SMILES: Cc1cnc(Nc2ccc(N3CCN(C(=O)OC(C)(C)C)CC3)c(Cl)c2)nc1] | 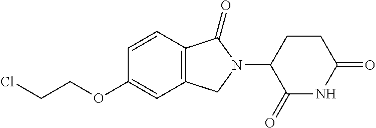 [SMILES: O=C1CCC(N2Cc3cc(OCCCl)ccc3C2=O)C(=O)N1] |
| 2-78 | 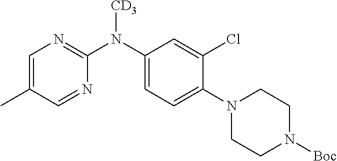 [SMILES: [2H]C([2H])([2H])N(c1ccc(N2CCN(C(=O)OC(C)(C)C)CC2)c(Cl)c1)c1ncc(C)cn1] | 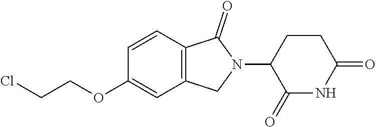 [SMILES: O=C1CCC(N2Cc3cc(OCCCl)ccc3C2=O)C(=O)N1] |
| 2-79 | 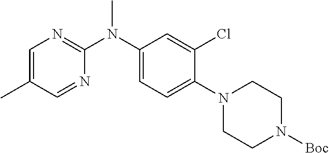 [SMILES: Cc1cnc(N(C)c2ccc(N3CCN(C(=O)OC(C)(C)C)CC3)c(Cl)c2)nc1] | 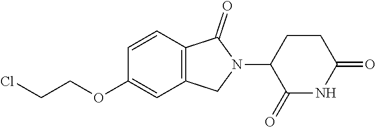 [SMILES: O=C1CCC(N2Cc3cc(OCCCl)ccc3C2=O)C(=O)N1] |
| 2-80 | 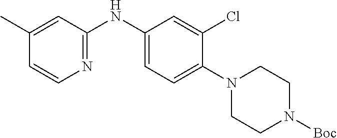 [SMILES: Cc1ccnc(Nc2ccc(N3CCN(C(=O)OC(C)(C)C)CC3)c(Cl)c2)c1] | 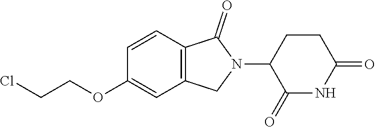 [SMILES: O=C1CCC(N2Cc3cc(OCCCl)ccc3C2=O)C(=O)N1] |
| 2-81 | 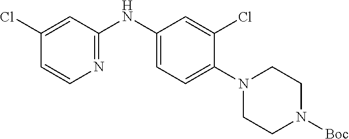 [SMILES: CC(C)(C)OC(=O)N1CCN(c2ccc(Nc3cc(Cl)ccn3)cc2Cl)CC1] | 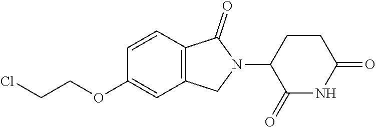 [SMILES: O=C1CCC(N2Cc3cc(OCCCl)ccc3C2=O)C(=O)N1] |
| 2-82 | 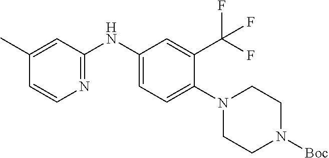 [SMILES: Cc1ccnc(Nc2ccc(N3CCN(C(=O)OC(C)(C)C)CC3)c(C(F)(F)F)c2)c1] | 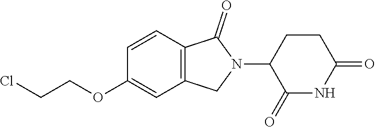 [SMILES: O=C1CCC(N2Cc3cc(OCCCl)ccc3C2=O)C(=O)N1] |
| 2-83 | 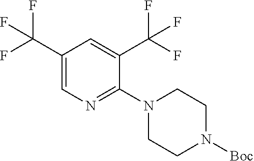 [SMILES: CC(C)(C)OC(=O)N1CCN(c2ncc(C(F)(F)F)cc2C(F)(F)F)CC1] | 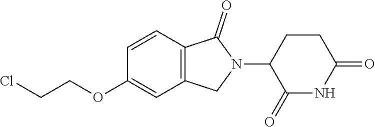 [SMILES: O=C1CCC(N2Cc3cc(OCCCl)ccc3C2=O)C(=O)N1] |
| 2-84 | 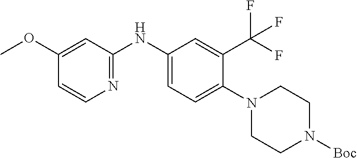 [SMILES: COc1ccnc(Nc2ccc(N3CCN(C(=O)OC(C)(C)C)CC3)c(C(F)(F)F)c2)c1] | 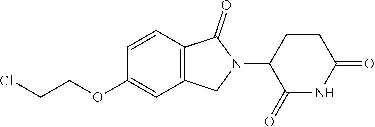 [SMILES: O=C1CCC(N2Cc3cc(OCCCl)ccc3C2=O)C(=O)N1] |
| 2-85 | 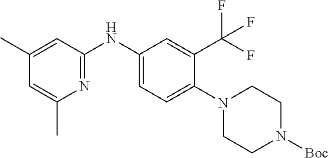 [SMILES: Cc1cc(C)nc(Nc2ccc(N3CCN(C(=O)OC(C)(C)C)CC3)c(C(F)(F)F)c2)c1] | 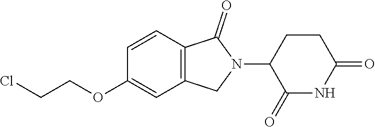 [SMILES: O=C1CCC(N2Cc3cc(OCCCl)ccc3C2=O)C(=O)N1] |
| 2-86 | 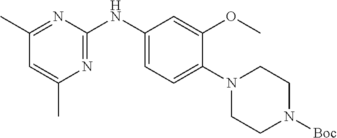 [SMILES: COc1cc(Nc2nc(C)cc(C)n2)ccc1N1CCN(C(=O)OC(C)(C)C)CC1] | 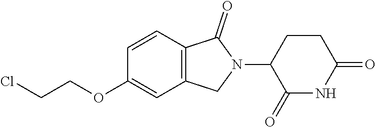 [SMILES: O=C1CCC(N2Cc3cc(OCCCl)ccc3C2=O)C(=O)N1] |
| 2-87 | 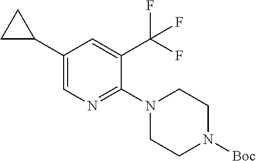 [SMILES: CC(C)(C)OC(=O)N1CCN(c2ncc(C3CC3)cc2C(F)(F)F)CC1] | 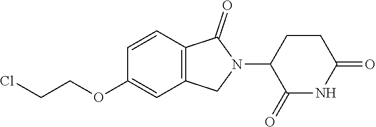 [SMILES: O=C1CCC(N2Cc3cc(OCCCl)ccc3C2=O)C(=O)N1] |
| 2-88 | 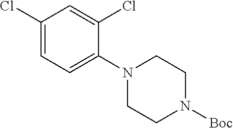 [SMILES: CC(C)(C)OC(=O)N1CCN(c2ccc(Cl)cc2Cl)CC1] | 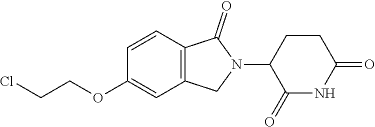 [SMILES: O=C1CCC(N2Cc3cc(OCCCl)ccc3C2=O)C(=O)N1] |
| 2-89 | 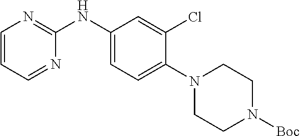 [SMILES: CC(C)(C)OC(=O)N1CCN(c2ccc(Nc3ncccn3)cc2Cl)CC1] | 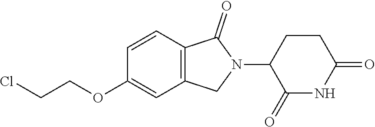 [SMILES: O=C1CCC(N2Cc3cc(OCCCl)ccc3C2=O)C(=O)N1] |
| 2-90 | 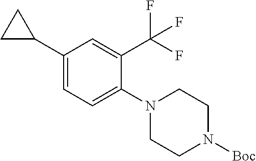 [SMILES: CC(C)(C)OC(=O)N1CCN(c2ccc(C3CC3)cc2C(F)(F)F)CC1] | 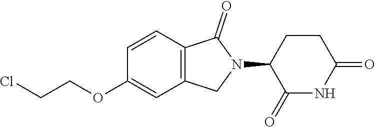 [SMILES: O=C1CC[C@H](N2Cc3cc(OCCCl)ccc3C2=O)C(=O)N1] |
| 2-91 | 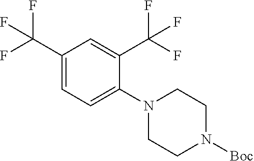 [SMILES: CC(C)(C)OC(=O)N1CCN(c2ccc(C(F)(F)F)cc2C(F)(F)F)CC1] | 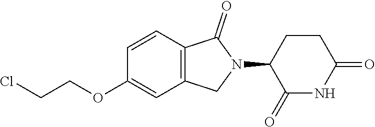 [SMILES: O=C1CC[C@H](N2Cc3cc(OCCCl)ccc3C2=O)C(=O)N1] |
| 2-92 | 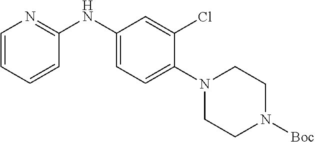 [SMILES: CC(C)(C)OC(=O)N1CCN(c2ccc(Nc3ccccn3)cc2Cl)CC1] | 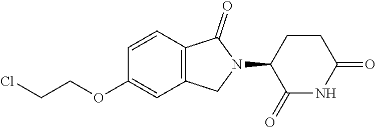 [SMILES: O=C1CC[C@H](N2Cc3cc(OCCCl)ccc3C2=O)C(=O)N1] |
| 2-93 | 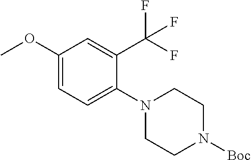 [SMILES: COc1ccc(N2CCN(C(=O)OC(C)(C)C)CC2)c(C(F)(F)F)c1] | 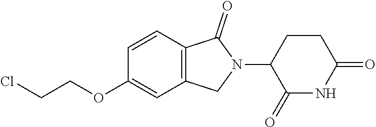 [SMILES: O=C1CCC(N2Cc3cc(OCCCl)ccc3C2=O)C(=O)N1] |
| 2-94 | 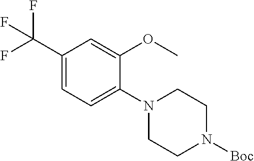 [SMILES: COc1cc(C(F)(F)F)ccc1N1CCN(C(=O)OC(C)(C)C)CC1] | 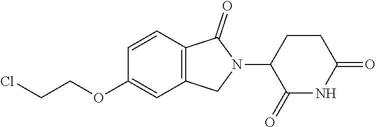 [SMILES: O=C1CCC(N2Cc3cc(OCCCl)ccc3C2=O)C(=O)N1] |
| 2-95 | 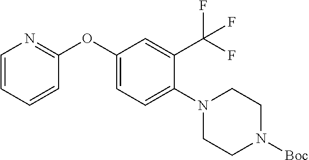 [SMILES: CC(C)(C)OC(=O)N1CCN(c2ccc(Oc3ccccn3)cc2C(F)(F)F)CC1] | 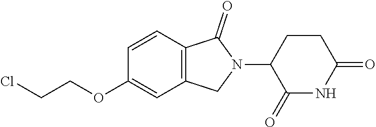 [SMILES: O=C1CCC(N2Cc3cc(OCCCl)ccc3C2=O)C(=O)N1] |
| 2-96 | 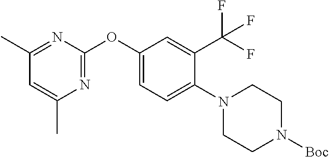 [SMILES: Cc1cc(C)nc(Oc2ccc(N3CCN(C(=O)OC(C)(C)C)CC3)c(C(F)(F)F)c2)n1] | 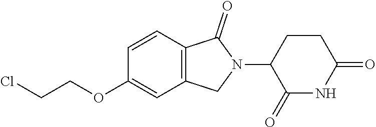 [SMILES: O=C1CCC(N2Cc3cc(OCCCl)ccc3C2=O)C(=O)N1] |
| 2-97 | 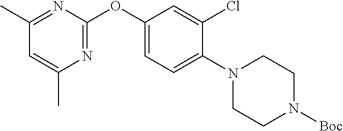 [SMILES: Cc1cc(C)nc(Oc2ccc(N3CCN(C(=O)OC(C)(C)C)CC3)c(Cl)c2)n1] | 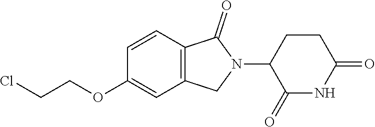 [SMILES: O=C1CCC(N2Cc3cc(OCCCl)ccc3C2=O)C(=O)N1] |
| 2-98 | 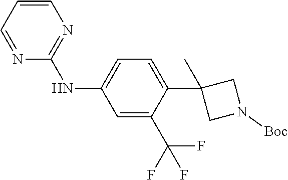 [SMILES: CC(C)(C)OC(=O)N1CC(C)(c2ccc(Nc3ncccn3)cc2C(F)(F)F)C1] | 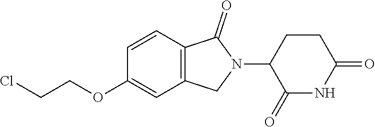 [SMILES: O=C1CCC(N2Cc3cc(OCCCl)ccc3C2=O)C(=O)N1] |
| 2-99 | 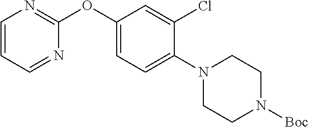 [SMILES: CC(C)(C)OC(=O)N1CCN(c2ccc(Oc3ncccn3)cc2Cl)CC1] | 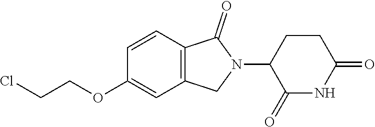 [SMILES: O=C1CCC(N2Cc3cc(OCCCl)ccc3C2=O)C(=O)N1] |
| 2-100 | 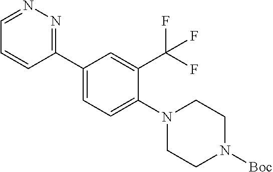 [SMILES: CC(C)(C)OC(=O)N1CCN(c2ccc(-c3cccnn3)cc2C(F)(F)F)CC1] | 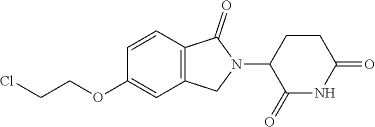 [SMILES: O=C1CCC(N2Cc3cc(OCCCl)ccc3C2=O)C(=O)N1] |
| 2-101 | 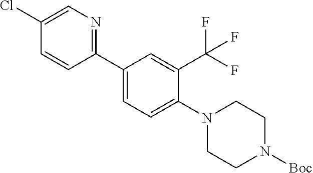 [SMILES: CC(C)(C)OC(=O)N1CCN(c2ccc(-c3ccc(Cl)cn3)cc2C(F)(F)F)CC1] | 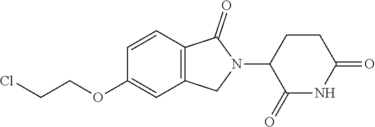 [SMILES: O=C1CCC(N2Cc3cc(OCCCl)ccc3C2=O)C(=O)N1] |
| 2-102 | 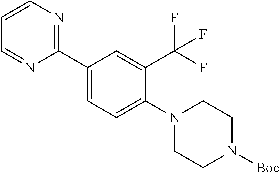 [SMILES: CC(C)(C)OC(=O)N1CCN(c2ccc(-c3ncccn3)cc2C(F)(F)F)CC1] | 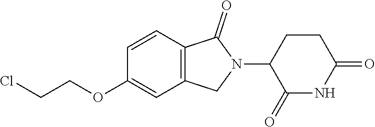 [SMILES: O=C1CCC(N2Cc3cc(OCCCl)ccc3C2=O)C(=O)N1] |
| 2-103 | 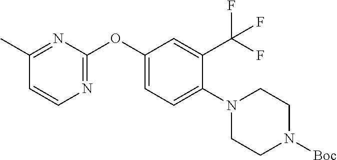 [SMILES: Cc1ccnc(Oc2ccc(N3CCN(C(=O)OC(C)(C)C)CC3)c(C(F)(F)F)c2)n1] | 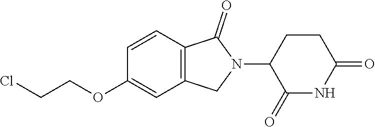 [SMILES: O=C1CCC(N2Cc3cc(OCCCl)ccc3C2=O)C(=O)N1] |
| 2-104 | 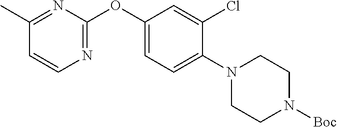 [SMILES: Cc1ccnc(Oc2ccc(N3CCN(C(=O)OC(C)(C)C)CC3)c(Cl)c2)n1] | 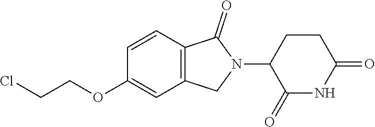 [SMILES: O=C1CCC(N2Cc3cc(OCCCl)ccc3C2=O)C(=O)N1] |
| 2-105 | 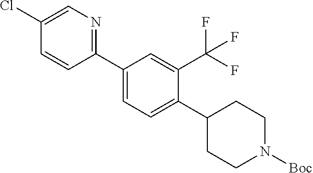 [SMILES: CC(C)(C)OC(=O)N1CCC(c2ccc(-c3ccc(Cl)cn3)cc2C(F)(F)F)CC1] | 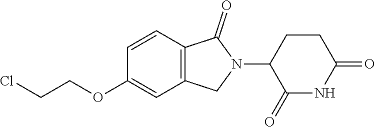 [SMILES: O=C1CCC(N2Cc3cc(OCCCl)ccc3C2=O)C(=O)N1] |
| 2-106 | 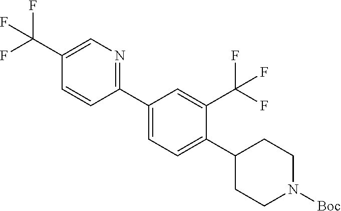 [SMILES: CC(C)(C)OC(=O)N1CCC(c2ccc(-c3ccc(C(F)(F)F)cn3)cc2C(F)(F)F)CC1] | 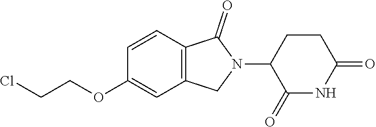 [SMILES: O=C1CCC(N2Cc3cc(OCCCl)ccc3C2=O)C(=O)N1] |
| 2-107 | 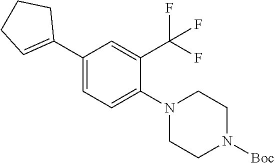 [SMILES: CC(C)(C)OC(=O)N1CCN(c2ccc(C3=CCCC3)cc2C(F)(F)F)CC1] | 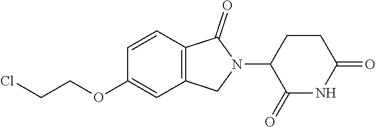 [SMILES: O=C1CCC(N2Cc3cc(OCCCl)ccc3C2=O)C(=O)N1] |
| 2-108 | 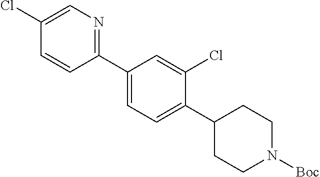 [SMILES: CC(C)(C)OC(=O)N1CCC(c2ccc(-c3ccc(Cl)cn3)cc2Cl)CC1] | 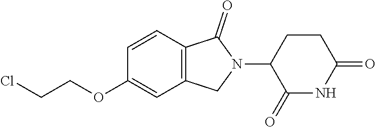 [SMILES: O=C1CCC(N2Cc3cc(OCCCl)ccc3C2=O)C(=O)N1] |
| 2-109 | 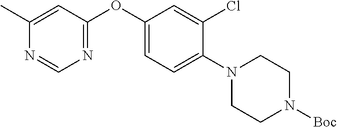 [SMILES: Cc1cc(Oc2ccc(N3CCN(C(=O)OC(C)(C)C)CC3)c(Cl)c2)ncn1] | 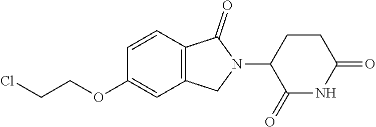 [SMILES: O=C1CCC(N2Cc3cc(OCCCl)ccc3C2=O)C(=O)N1] |
| 2-110 | 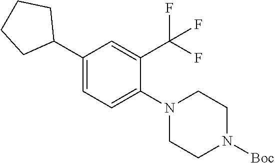 [SMILES: CC(C)(C)OC(=O)N1CCN(c2ccc(C3CCCC3)cc2C(F)(F)F)CC1] | 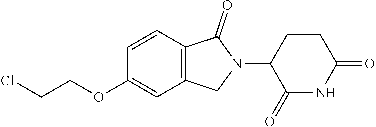 [SMILES: O=C1CCC(N2Cc3cc(OCCCl)ccc3C2=O)C(=O)N1] |
| 2-111 | 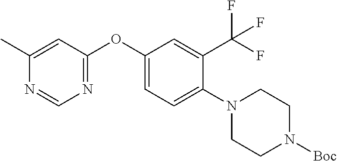 [SMILES: Cc1cc(Oc2ccc(N3CCN(C(=O)OC(C)(C)C)CC3)c(C(F)(F)F)c2)ncn1] | 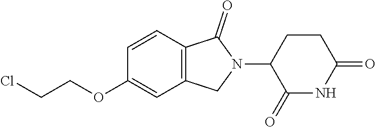 [SMILES: O=C1CCC(N2Cc3cc(OCCCl)ccc3C2=O)C(=O)N1] |
| 2-112 | 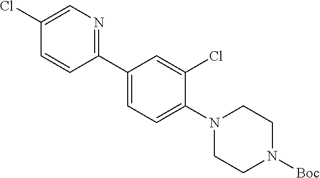 [SMILES: CC(C)(C)OC(=O)N1CCN(c2ccc(-c3ccc(Cl)cn3)cc2Cl)CC1] | 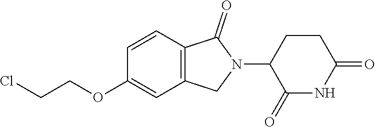 [SMILES: O=C1CCC(N2Cc3cc(OCCCl)ccc3C2=O)C(=O)N1] |
| 2-113 | 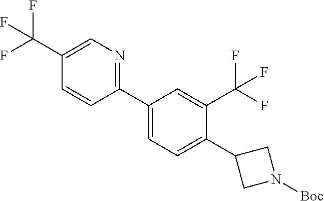 [SMILES: CC(C)(C)OC(=O)N1CC(c2ccc(-c3ccc(C(F)(F)F)cn3)cc2C(F)(F)F)C1] | 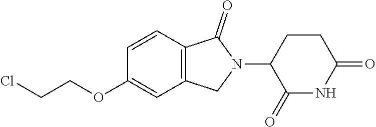 [SMILES: O=C1CCC(N2Cc3cc(OCCCl)ccc3C2=O)C(=O)N1] |
| 2-114 | 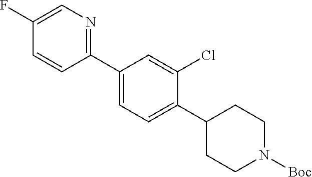 [SMILES: CC(C)(C)OC(=O)N1CCC(c2ccc(-c3ccc(F)cn3)cc2Cl)CC1] | 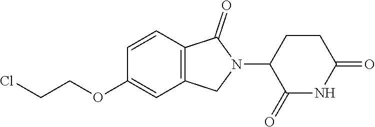 [SMILES: O=C1CCC(N2Cc3cc(OCCCl)ccc3C2=O)C(=O)N1] |
| 2-115 | 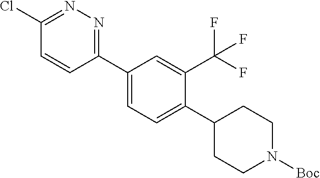 [SMILES: CC(C)(C)OC(=O)N1CCC(c2ccc(-c3ccc(Cl)nn3)cc2C(F)(F)F)CC1] | 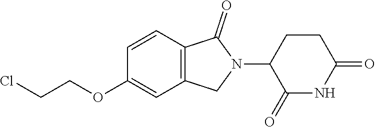 [SMILES: O=C1CCC(N2Cc3cc(OCCCl)ccc3C2=O)C(=O)N1] |
| 2-116 | 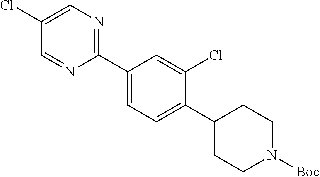 [SMILES: CC(C)(C)OC(=O)N1CCC(c2ccc(-c3ncc(Cl)cn3)cc2Cl)CC1] | 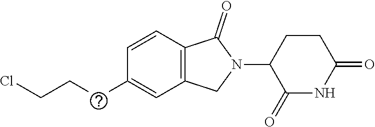 [SMILES: O=C1CCC(N2Cc3cc(CCCCl)ccc3C2=O)C(=O)N1] |
| 2-117 | 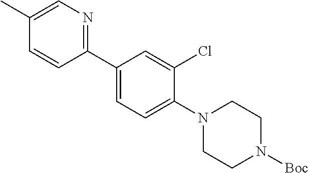 [SMILES: Cc1ccc(-c2ccc(N3CCN(C(=O)OC(C)(C)C)CC3)c(Cl)c2)nc1] | 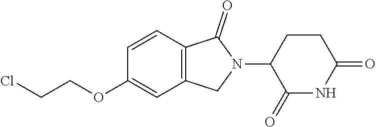 [SMILES: O=C1CCC(N2Cc3cc(OCCCl)ccc3C2=O)C(=O)N1] |
| 2-118 | 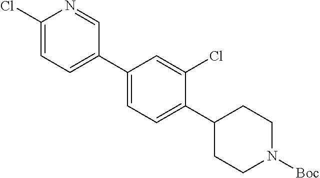 [SMILES: CC(C)(C)OC(=O)N1CCC(c2ccc(-c3ccc(Cl)nc3)cc2Cl)CC1] | 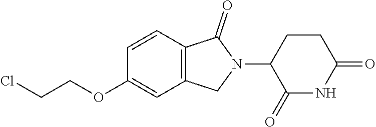 [SMILES: O=C1CCC(N2Cc3cc(OCCCl)ccc3C2=O)C(=O)N1] |
| 2-119 | 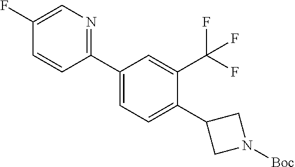 [SMILES: CC(C)(C)OC(=O)N1CC(c2ccc(-c3ccc(F)cn3)cc2C(F)(F)F)C1] | 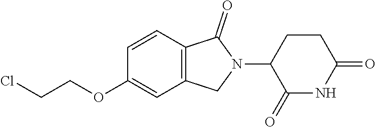 [SMILES: O=C1CCC(N2Cc3cc(OCCCl)ccc3C2=O)C(=O)N1] |
| 2-120 | 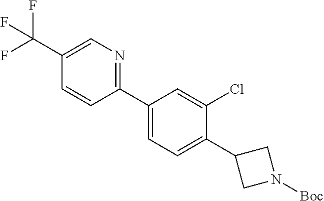 [SMILES: CC(C)(C)OC(=O)N1CC(c2ccc(-c3ccc(C(F)(F)F)cn3)cc2Cl)C1] | 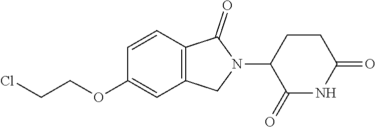 [SMILES: O=C1CCC(N2Cc3cc(OCCCl)ccc3C2=O)C(=O)N1] |
| 2-121 | 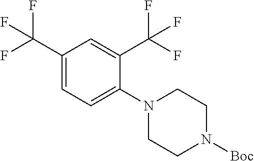 [SMILES: CC(C)(C)OC(=O)N1CCN(c2ccc(C(F)(F)F)cc2C(F)(F)F)CC1] | 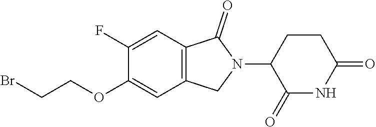 [SMILES: O=C1CCC(N2Cc3cc(OCCBr)c(F)cc3C2=O)C(=O)N1] |
| 2-122 | 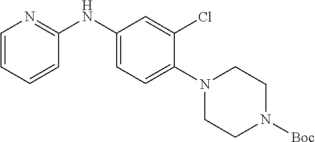 [SMILES: CC(C)(C)OC(=O)N1CCN(c2ccc(Nc3ccccn3)cc2Cl)CC1] | 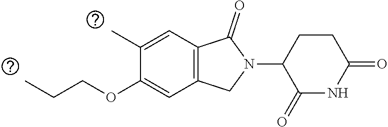 [SMILES: CCCOc1cc2c(cc1C)C(=O)N(C1CCC(=O)NC1=O)C2] |
| 2-123 | 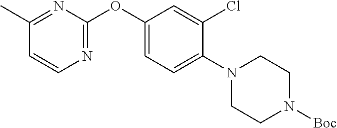 [SMILES: Cc1ccnc(Oc2ccc(N3CCN(C(=O)OC(C)(C)C)CC3)c(Cl)c2)n1] | 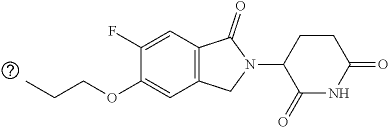 [SMILES: CCCOc1cc2c(cc1F)C(=O)N(C1CCC(=O)NC1=O)C2] |
| 2-124 | 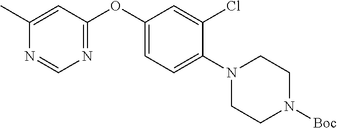 [SMILES: Cc1cc(Oc2ccc(N3CCN(C(=O)OC(C)(C)C)CC3)c(Cl)c2)ncn1] | 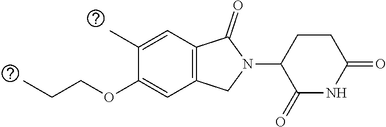 [SMILES: CCCOc1cc2c(cc1C)C(=O)N(C1CCC(=O)NC1=O)C2] |
| 2-125 | 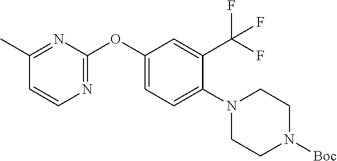 [SMILES: Cc1ccnc(Oc2ccc(N3CCN(C(=O)OC(C)(C)C)CC3)c(C(F)(F)F)c2)n1] | 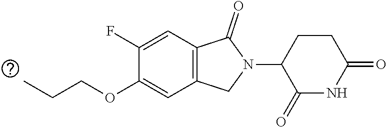 [SMILES: CCCOc1cc2c(cc1F)C(=O)N(C1CCC(=O)NC1=O)C2] |
| 2-126 | 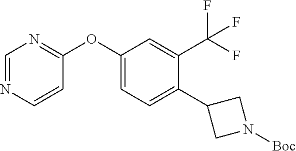 [SMILES: CC(C)(C)OC(=O)N1CC(c2ccc(Oc3ccncn3)cc2C(F)(F)F)C1] | 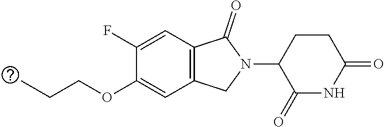 [SMILES: CCCOc1cc2c(cc1F)C(=O)N(C1CCC(=O)NC1=O)C2] |
| 2-127 | 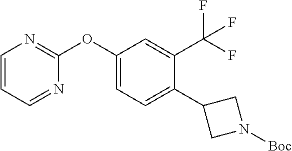 [SMILES: CC(C)(C)OC(=O)N1CC(c2ccc(Oc3ncccn3)cc2C(F)(F)F)C1] | 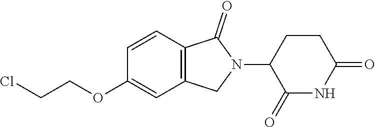 [SMILES: O=C1CCC(N2Cc3cc(OCCCl)ccc3C2=O)C(=O)N1] |
| 2-128 | 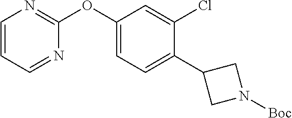 [SMILES: CC(C)(C)OC(=O)N1CC(c2ccc(Oc3ncccn3)cc2Cl)C1] | 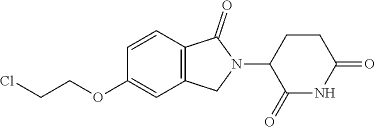 [SMILES: O=C1CCC(N2Cc3cc(OCCCl)ccc3C2=O)C(=O)N1] |
| 2-129 | 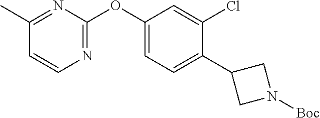 [SMILES: Cc1ccnc(Oc2ccc(C3CN(C(=O)OC(C)(C)C)C3)c(Cl)c2)n1] | 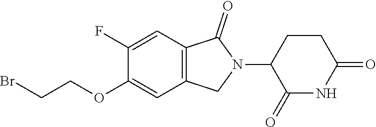 [SMILES: O=C1CCC(N2Cc3cc(OCCBr)c(F)cc3C2=O)C(=O)N1] |
| 2-130 | 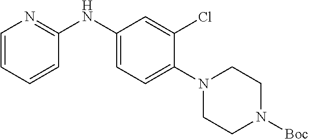 [SMILES: CC(C)(C)OC(=O)N1CCN(c2ccc(Nc3ccccn3)cc2Cl)CC1] | 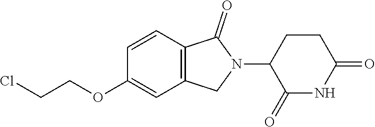 [SMILES: O=C1CCC(N2Cc3cc(OCCCl)ccc3C2=O)C(=O)N1] |
| 2-131 | 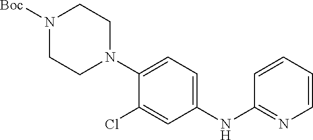 [SMILES: CC(C)(C)OC(=O)N1CCN(c2ccc(Nc3ccccn3)cc2Cl)CC1] | 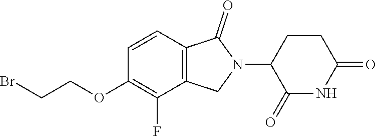 [SMILES: O=C1CCC(N2Cc3c(ccc(OCCBr)c3F)C2=O)C(=O)N1] |
| 2-132 | 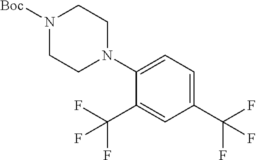 [SMILES: CC(C)(C)OC(=O)N1CCN(c2ccc(C(F)(F)F)cc2C(F)(F)F)CC1] | 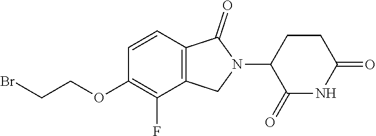 [SMILES: O=C1CCC(N2Cc3c(ccc(OCCBr)c3F)C2=O)C(=O)N1] |
| 2-133 | 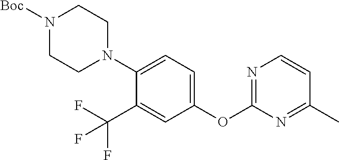 [SMILES: Cc1ccnc(Oc2ccc(N3CCN(C(=O)OC(C)(C)C)CC3)c(C(F)(F)F)c2)n1] | 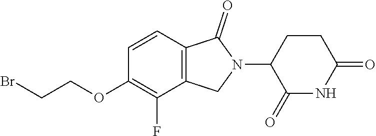 [SMILES: O=C1CCC(N2Cc3c(ccc(OCCBr)c3F)C2=O)C(=O)N1] |
| 2-134 | 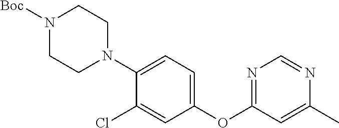 [SMILES: Cc1cc(Oc2ccc(N3CCN(C(=O)OC(C)(C)C)CC3)c(Cl)c2)ncn1] | 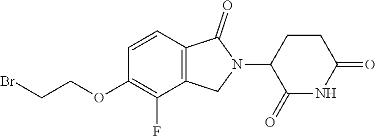 [SMILES: O=C1CCC(N2Cc3c(ccc(OCCBr)c3F)C2=O)C(=O)N1] |
| 2-135 | 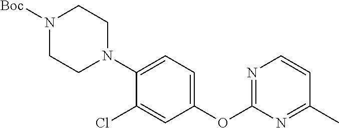 [SMILES: Cc1ccnc(Oc2ccc(N3CCN(C(=O)OC(C)(C)C)CC3)c(Cl)c2)n1] | 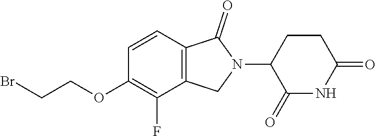 [SMILES: O=C1CCC(N2Cc3c(ccc(OCCBr)c3F)C2=O)C(=O)N1] |
| 2-136 | 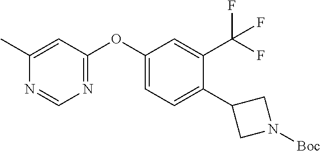 [SMILES: Cc1cc(Oc2ccc(C3CN(C(=O)OC(C)(C)C)C3)c(C(F)(F)F)c2)ncn1] | 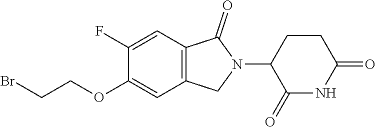 [SMILES: O=C1CCC(N2Cc3cc(OCCBr)c(F)cc3C2=O)C(=O)N1] |
| 2-137 | 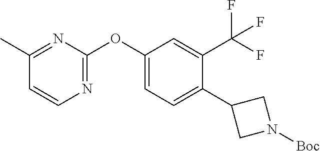 [SMILES: Cc1ccnc(Oc2ccc(C3CN(C(=O)OC(C)(C)C)C3)c(C(F)(F)F)c2)n1] | 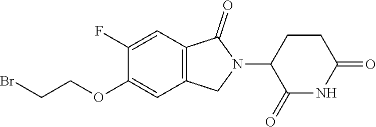 [SMILES: O=C1CCC(N2Cc3cc(OCCBr)c(F)cc3C2=O)C(=O)N1] |
| 2-138 | 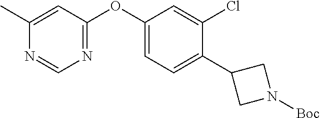 [SMILES: Cc1cc(Oc2ccc(C3CN(C(=O)OC(C)(C)C)C3)c(Cl)c2)ncn1] | 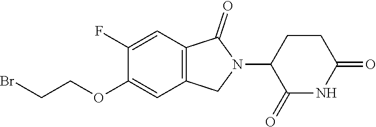 [SMILES: O=C1CCC(N2Cc3cc(OCCBr)c(F)cc3C2=O)C(=O)N1] |
| 2-139 | 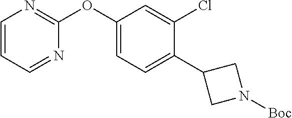 [SMILES: CC(C)(C)OC(=O)N1CC(c2ccc(Oc3ncccn3)cc2Cl)C1] | 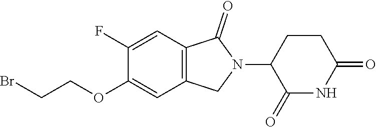 [SMILES: O=C1CCC(N2Cc3cc(OCCBr)c(F)cc3C2=O)C(=O)N1] |
| 2-140 | 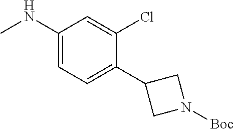 [SMILES: CNc1ccc(C2CN(C(=O)OC(C)(C)C)C2)c(Cl)c1] | 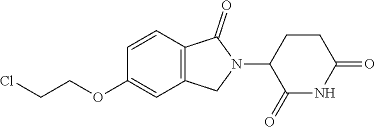 [SMILES: O=C1CCC(N2Cc3cc(OCCCl)ccc3C2=O)C(=O)N1] |
| 2-141 | 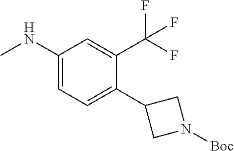 [SMILES: CNc1ccc(C2CN(C(=O)OC(C)(C)C)C2)c(C(F)(F)F)c1] | 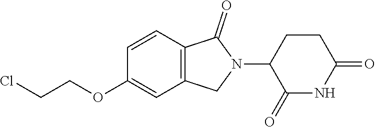 [SMILES: O=C1CCC(N2Cc3cc(OCCCl)ccc3C2=O)C(=O)N1] |
| 2-142 | 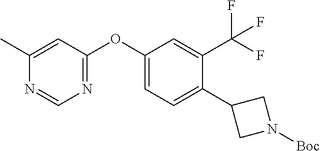 [SMILES: Cc1cc(Oc2ccc(C3CN(C(=O)OC(C)(C)C)C3)c(C(F)(F)F)c2)ncn1] | 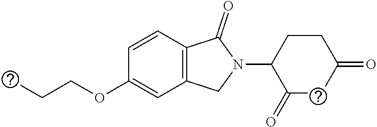 [SMILES: CCCOc1ccc2c(c1)CN(C1CCC(=O)CC1=O)C2=O] |
| 2-143 | 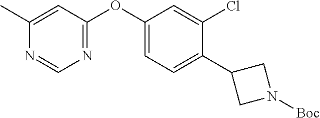 [SMILES: Cc1cc(Oc2ccc(C3CN(C(=O)OC(C)(C)C)C3)c(Cl)c2)ncn1] | 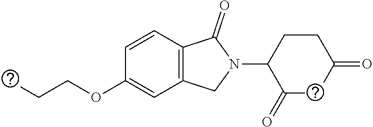 [SMILES: CCCOc1ccc2c(c1)CN(C1CCC(=O)CC1=O)C2=O] |
| 2-144 | 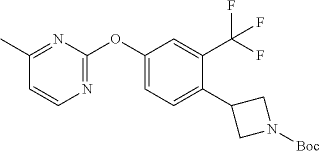 [SMILES: Cc1ccnc(Oc2ccc(C3CN(C(=O)OC(C)(C)C)C3)c(C(F)(F)F)c2)n1] | 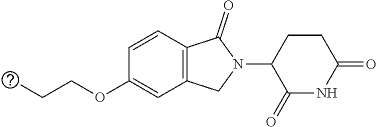 [SMILES: CCCOc1ccc2c(c1)CN(C1CCC(=O)NC1=O)C2=O] |
| 2-145 | 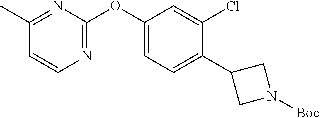 [SMILES: Cc1ccnc(Oc2ccc(C3CN(C(=O)OC(C)(C)C)C3)c(Cl)c2)n1] | 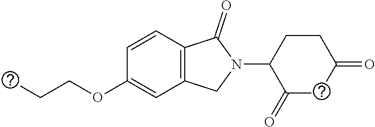 [SMILES: CCCOc1ccc2c(c1)CN(C1CCC(=O)CC1=O)C2=O] |
| 2-146 | 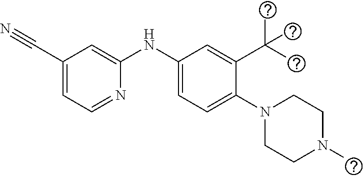 [SMILES: CN1CCN(c2ccc(Nc3cc(C#N)ccn3)cc2C(C)(C)C)CC1] | 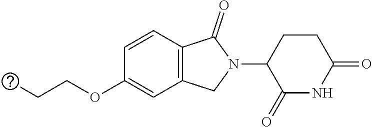 [SMILES: CCCOc1ccc2c(c1)CN(C1CCC(=O)NC1=O)C2=O] |
| 2-147 | 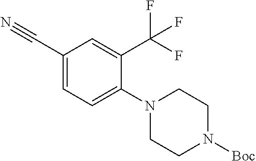 [SMILES: CC(C)(C)OC(=O)N1CCN(c2ccc(C#N)cc2C(F)(F)F)CC1] | 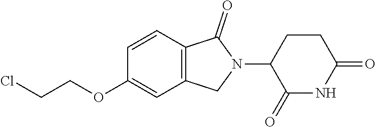 [SMILES: O=C1CCC(N2Cc3cc(OCCCl)ccc3C2=O)C(=O)N1] |
| 2-148 | 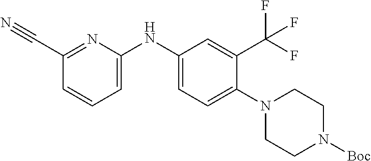 [SMILES: CC(C)(C)OC(=O)N1CCN(c2ccc(Nc3cccc(C#N)n3)cc2C(F)(F)F)CC1] | 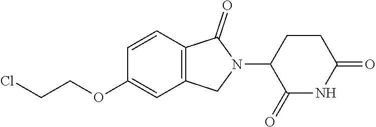 [SMILES: O=C1CCC(N2Cc3cc(OCCCl)ccc3C2=O)C(=O)N1] |
| 2-149 | 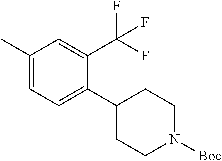 [SMILES: Cc1ccc(C2CCN(C(=O)OC(C)(C)C)CC2)c(C(F)(F)F)c1] | 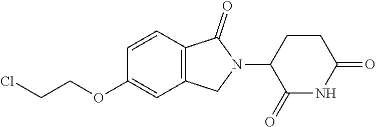 [SMILES: O=C1CCC(N2Cc3cc(OCCCl)ccc3C2=O)C(=O)N1] |
| 2-150 | 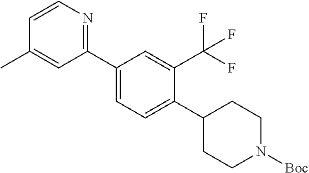 [SMILES: Cc1ccnc(-c2ccc(C3CCN(C(=O)OC(C)(C)C)CC3)c(C(F)(F)F)c2)c1] | 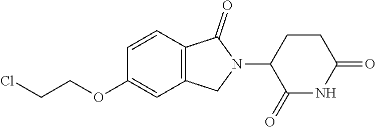 [SMILES: O=C1CCC(N2Cc3cc(OCCCl)ccc3C2=O)C(=O)N1] |
| 2-151 | 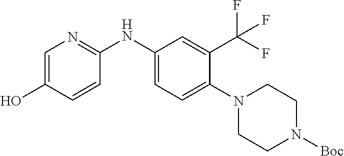 [SMILES: CC(C)(C)OC(=O)N1CCN(c2ccc(Nc3ccc(O)cn3)cc2C(F)(F)F)CC1] | 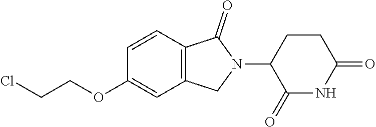 [SMILES: O=C1CCC(N2Cc3cc(OCCCl)ccc3C2=O)C(=O)N1] |
| 2-152 | 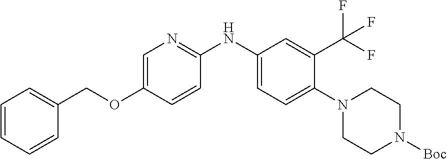 [SMILES: CC(C)(C)OC(=O)N1CCN(c2ccc(Nc3ccc(OCc4ccccc4)cn3)cc2C(F)(F)F)CC1] | 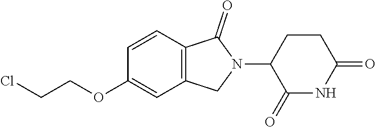 [SMILES: O=C1CCC(N2Cc3cc(OCCCl)ccc3C2=O)C(=O)N1] |
| 2-153 | 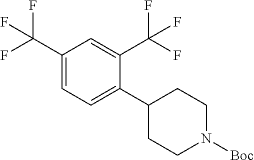 [SMILES: CC(C)(C)OC(=O)N1CCC(c2ccc(C(F)(F)F)cc2C(F)(F)F)CC1] | 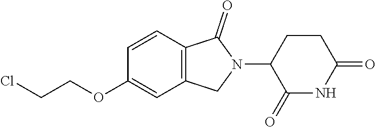 [SMILES: O=C1CCC(N2Cc3cc(OCCCl)ccc3C2=O)C(=O)N1] |
| 2-154 | 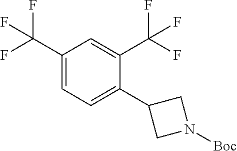 [SMILES: CC(C)(C)OC(=O)N1CC(c2ccc(C(F)(F)F)cc2C(F)(F)F)C1] | 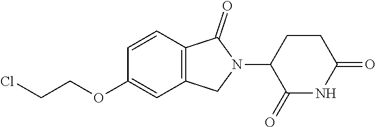 [SMILES: O=C1CCC(N2Cc3cc(OCCCl)ccc3C2=O)C(=O)N1] |
| 2-155 | 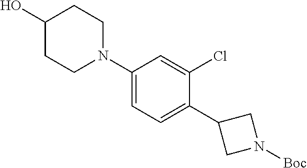 [SMILES: CC(C)(C)OC(=O)N1CC(c2ccc(N3CCC(O)CC3)cc2Cl)C1] | 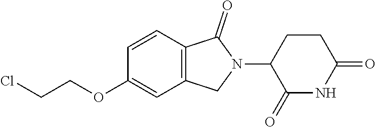 [SMILES: O=C1CCC(N2Cc3cc(OCCCl)ccc3C2=O)C(=O)N1] |
| 2-156 | 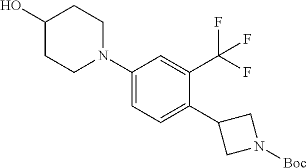 [SMILES: CC(C)(C)OC(=O)N1CC(c2ccc(N3CCC(O)CC3)cc2C(F)(F)F)C1] | 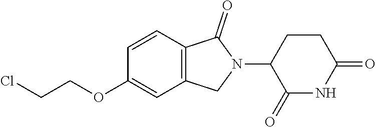 [SMILES: O=C1CCC(N2Cc3cc(OCCCl)ccc3C2=O)C(=O)N1] |
| 2-157 | 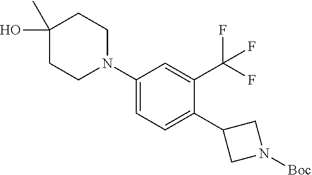 [SMILES: CC1(O)CCN(c2ccc(C3CN(C(=O)OC(C)(C)C)C3)c(C(F)(F)F)c2)CC1] | 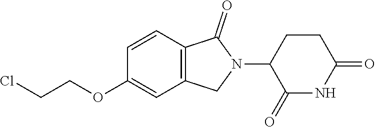 [SMILES: O=C1CCC(N2Cc3cc(OCCCl)ccc3C2=O)C(=O)N1] |
| 2-158 | 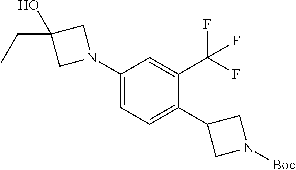 [SMILES: CCC1(O)CN(c2ccc(C3CN(C(=O)OC(C)(C)C)C3)c(C(F)(F)F)c2)C1] | 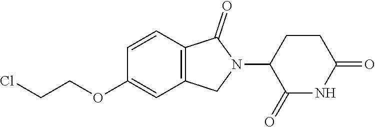 [SMILES: O=C1CCC(N2Cc3cc(OCCCl)ccc3C2=O)C(=O)N1] |
| 2-159 | 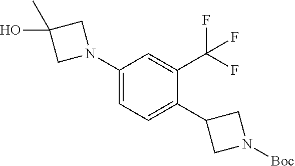 [SMILES: CC1(O)CN(c2ccc(C3CN(C(=O)OC(C)(C)C)C3)c(C(F)(F)F)c2)C1] | 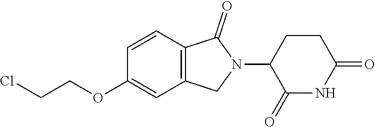 [SMILES: O=C1CCC(N2Cc3cc(OCCCl)ccc3C2=O)C(=O)N1] |
| 2-160 | 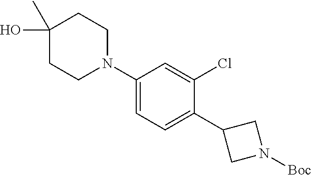 [SMILES: CC1(O)CCN(c2ccc(C3CN(C(=O)OC(C)(C)C)C3)c(Cl)c2)CC1] | 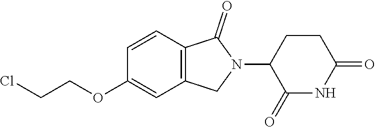 [SMILES: O=C1CCC(N2Cc3cc(OCCCl)ccc3C2=O)C(=O)N1] |
| 2-161 | 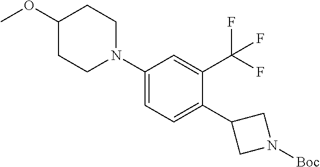 [SMILES: COC1CCN(c2ccc(C3CN(C(=O)OC(C)(C)C)C3)c(C(F)(F)F)c2)CC1] | 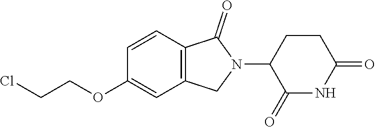 [SMILES: O=C1CCC(N2Cc3cc(OCCCl)ccc3C2=O)C(=O)N1] |
| 2-162 | 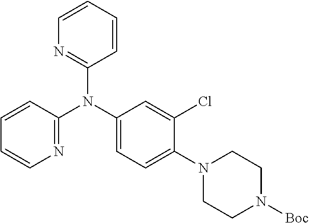 [SMILES: CC(C)(C)OC(=O)N1CCN(c2ccc(N(c3ccccn3)c3ccccn3)cc2Cl)CC1] | 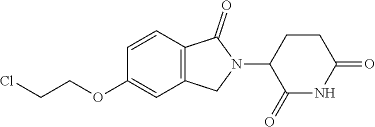 [SMILES: O=C1CCC(N2Cc3cc(OCCCl)ccc3C2=O)C(=O)N1] |
| 2-163 | 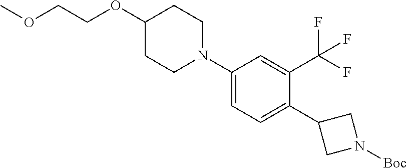 [SMILES: COCCOC1CCN(c2ccc(C3CN(C(=O)OC(C)(C)C)C3)c(C(F)(F)F)c2)CC1] | 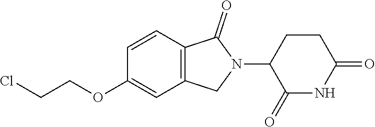 [SMILES: O=C1CCC(N2Cc3cc(OCCCl)ccc3C2=O)C(=O)N1] |
| 2-164 | 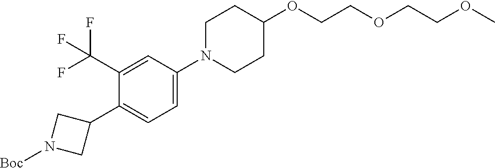 [SMILES: COCCOCCOC1CCN(c2ccc(C3CN(C(=O)OC(C)(C)C)C3)c(C(F)(F)F)c2)CC1] | 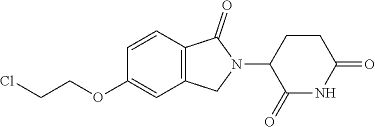 [SMILES: O=C1CCC(N2Cc3cc(OCCCl)ccc3C2=O)C(=O)N1] |
| 2-165 | 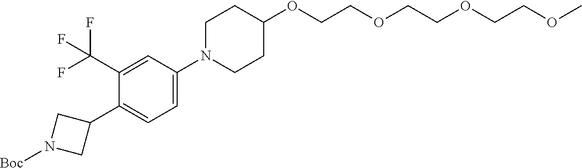 [SMILES: COCCOCCOCCOC1CCN(c2ccc(C3CN(C(=O)OC(C)(C)C)C3)c(C(F)(F)F)c2)CC1] | 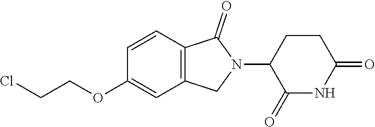 [SMILES: O=C1CCC(N2Cc3cc(OCCCl)ccc3C2=O)C(=O)N1] |
| 2-166 | 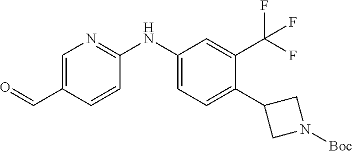 [SMILES: CC(C)(C)OC(=O)N1CC(c2ccc(Nc3ccc(C=O)cn3)cc2C(F)(F)F)C1] | 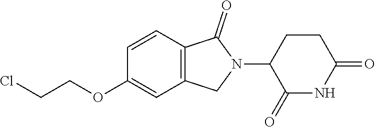 [SMILES: O=C1CCC(N2Cc3cc(OCCCl)ccc3C2=O)C(=O)N1] |
| 2-168 | 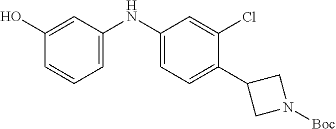 [SMILES: CC(C)(C)OC(=O)N1CC(c2ccc(Nc3cccc(O)c3)cc2Cl)C1] | 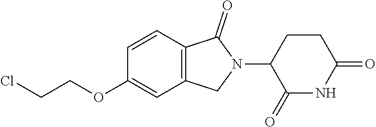 [SMILES: O=C1CCC(N2Cc3cc(OCCCl)ccc3C2=O)C(=O)N1] |
| 2-169 | 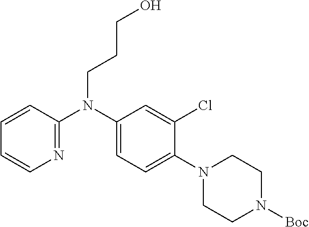 [SMILES: CC(C)(C)OC(=O)N1CCN(c2ccc(N(CCCO)c3ccccn3)cc2Cl)CC1] | 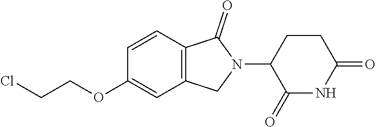 [SMILES: O=C1CCC(N2Cc3cc(OCCCl)ccc3C2=O)C(=O)N1] |
| 2-170 | 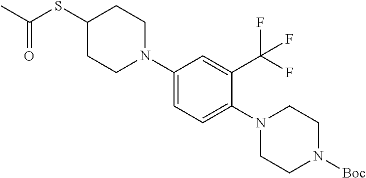 [SMILES: CC(=O)SC1CCN(c2ccc(N3CCN(C(=O)OC(C)(C)C)CC3)c(C(F)(F)F)c2)CC1] | 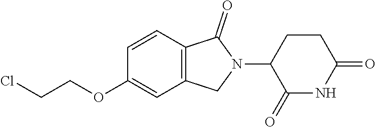 [SMILES: O=C1CCC(N2Cc3cc(OCCCl)ccc3C2=O)C(=O)N1] |
| 2-171 | 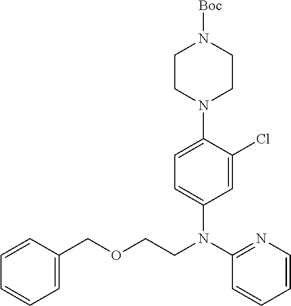 [SMILES: CC(C)(C)OC(=O)N1CCN(c2ccc(N(CCOCc3ccccc3)c3ccccn3)cc2Cl)CC1] | 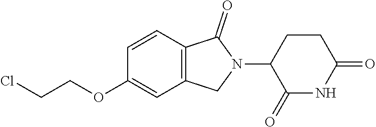 [SMILES: O=C1CCC(N2Cc3cc(OCCCl)ccc3C2=O)C(=O)N1] |
| 2-172 | 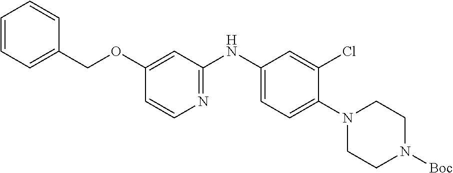 [SMILES: CC(C)(C)OC(=O)N1CCN(c2ccc(Nc3cc(OCc4ccccc4)ccn3)cc2Cl)CC1] | 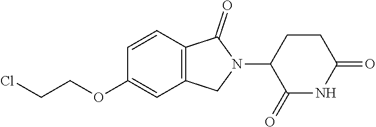 [SMILES: O=C1CCC(N2Cc3cc(OCCCl)ccc3C2=O)C(=O)N1] |
| 2-173 | 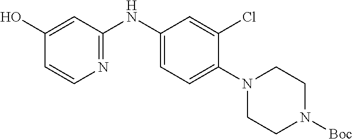 [SMILES: CC(C)(C)OC(=O)N1CCN(c2ccc(Nc3cc(O)ccn3)cc2Cl)CC1] | 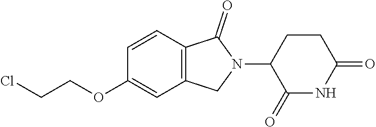 [SMILES: O=C1CCC(N2Cc3cc(OCCCl)ccc3C2=O)C(=O)N1] |
| 2-174 | 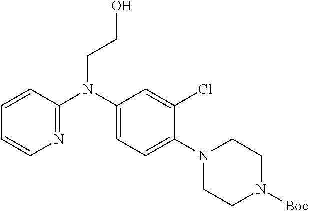 [SMILES: CC(C)(C)OC(=O)N1CCN(c2ccc(N(CCO)c3ccccn3)cc2Cl)CC1] | 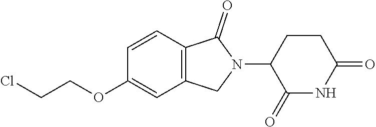 [SMILES: O=C1CCC(N2Cc3cc(OCCCl)ccc3C2=O)C(=O)N1] |
| 2-175 | 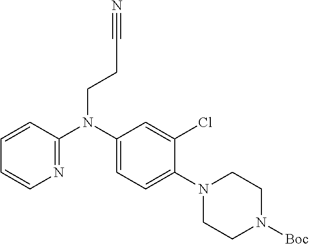 [SMILES: CC(C)(C)OC(=O)N1CCN(c2ccc(N(CCC#N)c3ccccn3)cc2Cl)CC1] | 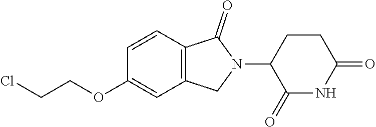 [SMILES: O=C1CCC(N2Cc3cc(OCCCl)ccc3C2=O)C(=O)N1] |
| 2-176 | 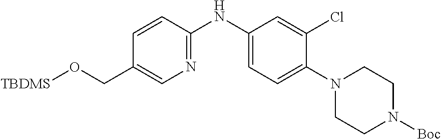 | 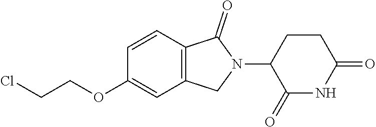 [SMILES: O=C1CCC(N2Cc3cc(OCCCl)ccc3C2=O)C(=O)N1] |
| 2-177 | 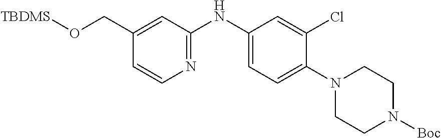 | 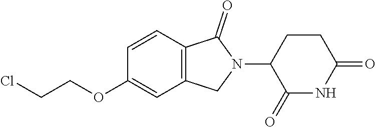 [SMILES: O=C1CCC(N2Cc3cc(OCCCl)ccc3C2=O)C(=O)N1] |
| 2-178 | 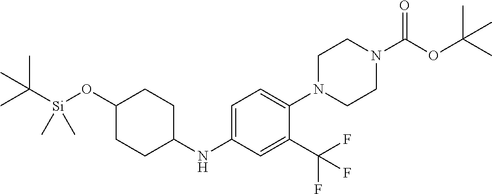 [SMILES: CC(C)(C)OC(=O)N1CCN(c2ccc(NC3CCC(O[Si](C)(C)C(C)(C)C)CC3)cc2C(F)(F)F)CC1] | 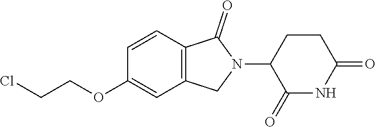 [SMILES: O=C1CCC(N2Cc3cc(OCCCl)ccc3C2=O)C(=O)N1] |
| 2-179 | 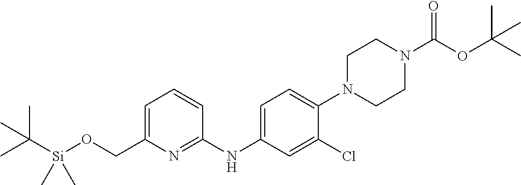 [SMILES: CC(C)(C)OC(=O)N1CCN(c2ccc(Nc3cccc(CO[Si](C)(C)C(C)(C)C)n3)cc2Cl)CC1] | 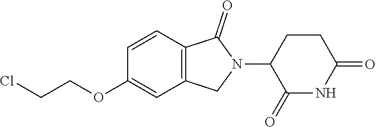 [SMILES: O=C1CCC(N2Cc3cc(OCCCl)ccc3C2=O)C(=O)N1] |
| 2-180 | 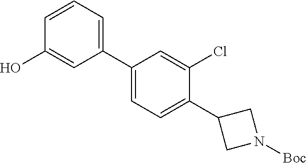 [SMILES: CC(C)(C)OC(=O)N1CC(c2ccc(-c3cccc(O)c3)cc2Cl)C1] | 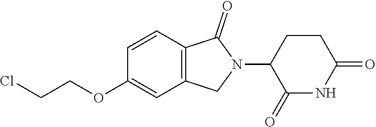 [SMILES: O=C1CCC(N2Cc3cc(OCCCl)ccc3C2=O)C(=O)N1] |
| 2-181 | 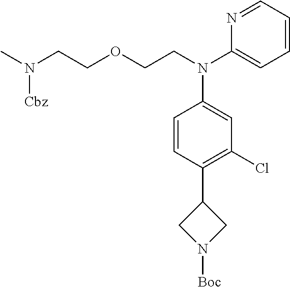 [SMILES: CN(CCOCCN(c1ccc(C2CN(C(=O)OC(C)(C)C)C2)c(Cl)c1)c1ccccn1)C(=O)OCc1ccccc1] | 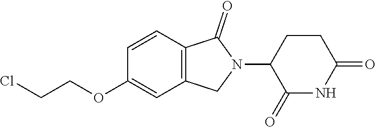 [SMILES: O=C1CCC(N2Cc3cc(OCCCl)ccc3C2=O)C(=O)N1] |
| 2-182 | 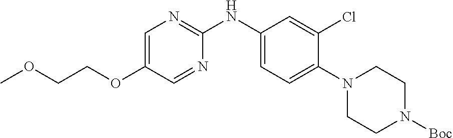 [SMILES: COCCOc1cnc(Nc2ccc(N3CCN(C(=O)OC(C)(C)C)CC3)c(Cl)c2)nc1] | 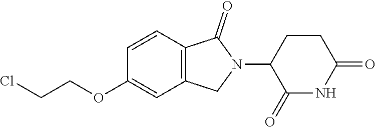 [SMILES: O=C1CCC(N2Cc3cc(OCCCl)ccc3C2=O)C(=O)N1] |
| 2-183 | 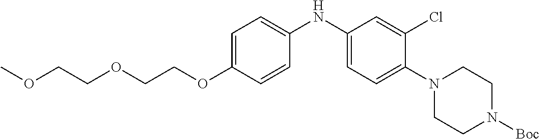 [SMILES: COCCOCCOc1ccc(Nc2ccc(N3CCN(C(=O)OC(C)(C)C)CC3)c(Cl)c2)cc1] | 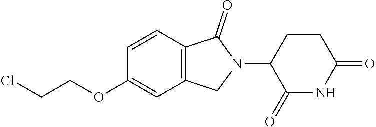 [SMILES: O=C1CCC(N2Cc3cc(OCCCl)ccc3C2=O)C(=O)N1] |
| 2-184 | 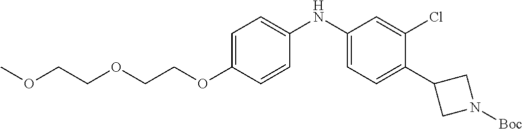 [SMILES: COCCOCCOc1ccc(Nc2ccc(C3CN(C(=O)OC(C)(C)C)C3)c(Cl)c2)cc1] | 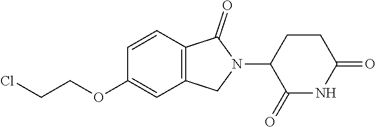 [SMILES: O=C1CCC(N2Cc3cc(OCCCl)ccc3C2=O)C(=O)N1] |
| 2-185 | 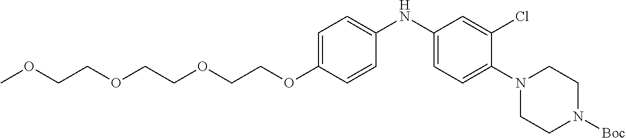 [SMILES: COCCOCCOCCOc1ccc(Nc2ccc(N3CCN(C(=O)OC(C)(C)C)CC3)c(Cl)c2)cc1] | 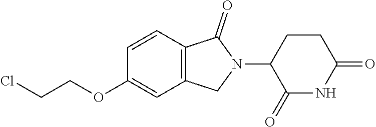 [SMILES: O=C1CCC(N2Cc3cc(OCCCl)ccc3C2=O)C(=O)N1] |
| 2-186 | 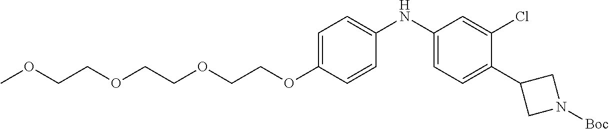 [SMILES: COCCOCCOCCOc1ccc(Nc2ccc(C3CN(C(=O)OC(C)(C)C)C3)c(Cl)c2)cc1] | 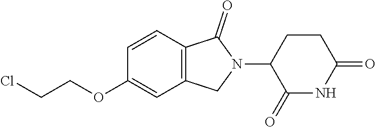 [SMILES: O=C1CCC(N2Cc3cc(OCCCl)ccc3C2=O)C(=O)N1] |
| 2-187 | 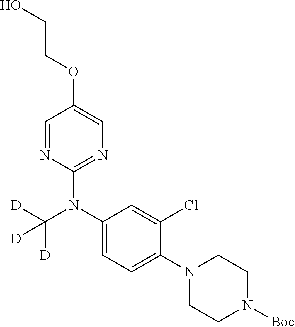 [SMILES: [2H]C([2H])([2H])N(c1ccc(N2CCN(C(=O)OC(C)(C)C)CC2)c(Cl)c1)c1ncc(OCCO)cn1] | 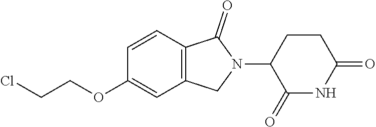 [SMILES: O=C1CCC(N2Cc3cc(OCCCl)ccc3C2=O)C(=O)N1] |
| | | MS |
| | | found |
| Compd | Product | [M + H]+ |
| | 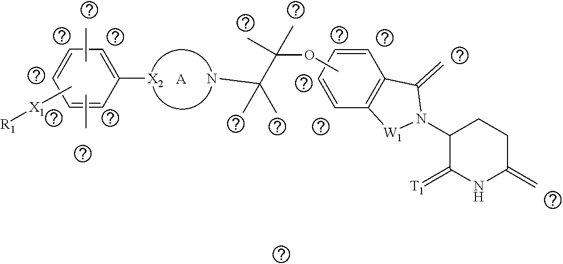 | |
| 2-1 | 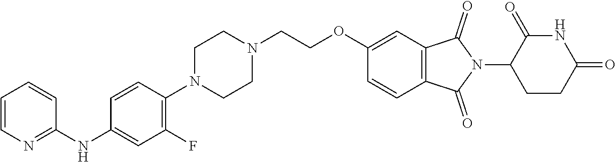 [SMILES: O=C1CCC(N2C(=O)c3ccc(OCCN4CCN(c5ccc(Nc6ccccn6)cc5F)CC4)cc3C2=O)C(=O)N1] | 573.4 |
| 2-2 | 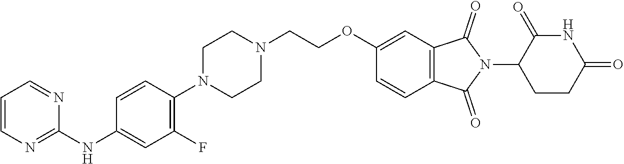 [SMILES: O=C1CCC(N2C(=O)c3ccc(OCCN4CCN(c5ccc(Nc6ncccn6)cc5F)CC4)cc3C2=O)C(=O)N1] | 574.4 |
| 2-3 | 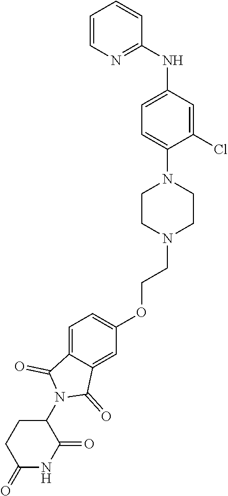 [SMILES: O=C1CCC(N2C(=O)c3ccc(OCCN4CCN(c5ccc(Nc6ccccn6)cc5Cl)CC4)cc3C2=O)C(=O)N1] | 589.3 |
| 2-4 | 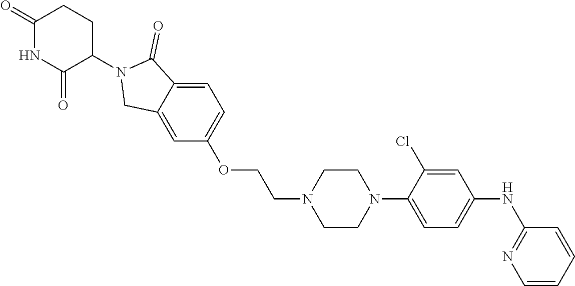 [SMILES: O=C1CCC(N2Cc3cc(OCCN4CCN(c5ccc(Nc6ccccn6)cc5Cl)CC4)ccc3C2=O)C(=O)N1] | 575.4 |
| | ¹H NMR (400 MHz, DMSO-d6) δ ppm 1.93-2.04 (m, 1H), 2.30- | |
| | 2.45 (m, 1H), 2.53-2.74 (m, 5H), 2.81 (t, J = 5.2 Hz, 2H), 2.84- | |
| | 3.01 (m, 5H), 4.15-4.32 (m, 3H), 4.39 (d, J = 17.2 Hz, 1H), 5.08 | |
| | (dd, J = 13.6, 5.2 Hz, 1H), 6.68-6.82 (m, 2H), 7.04-7.12 (m, 2H), | |
| | 7.21 (br s, 1H), 7.45 (dd, J = 8.8, 2.4 Hz, 1H), 7.51-7.58 (m, 1H), | |
| | 7.63 (d, J = 8.4 Hz, 1H), 7.96 (d, J = 2.4 Hz, 1H), 8.14 (d, J = 3.6 | |
| | Hz, 1H), 9.05 (br s, 1H), 10.97 (br s, 1H) | |
| 2-5 | 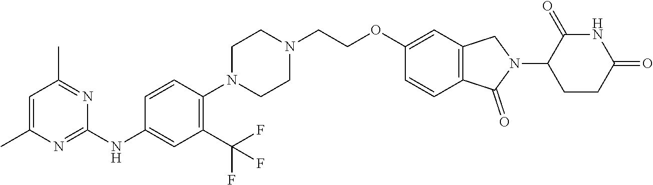 [SMILES: Cc1cc(C)nc(Nc2ccc(N3CCN(CCOc4ccc5c(c4)CN(C4CCC(=O)NC4=O)C5=O)CC3)c(C(F)(F)F)c2)n1] | 638.5 |
| 2-6 | 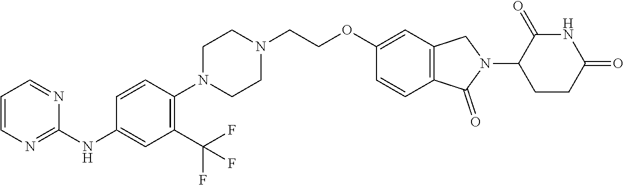 [SMILES: O=C1CCC(N2Cc3cc(OCCN4CCN(c5ccc(Nc6ncccn6)cc5C(F)(F)F)CC4)ccc3C2=O)C(=O)N1] | 610.4 |
| 2-7 | 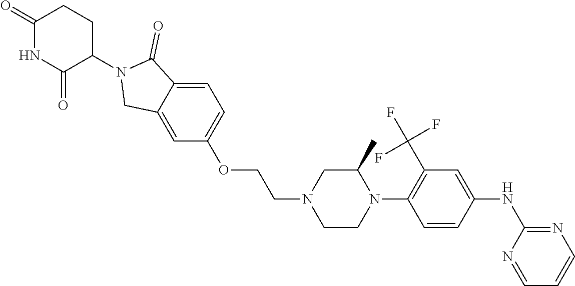 [SMILES: C[C@@H]1CN(CCOc2ccc3c(c2)CN(C2CCC(=O)NC2=O)C3=O)CCN1c1ccc(Nc2ncccn2)cc1C(F)(F)F] | 624.5 |
| 2-8 | 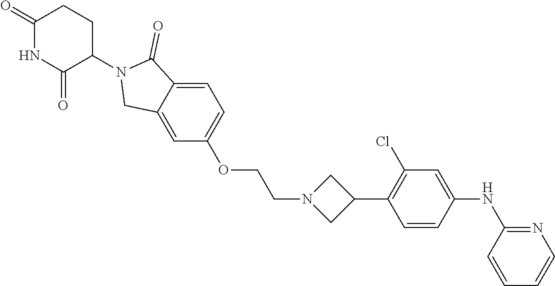 [SMILES: O=C1CCC(N2Cc3cc(OCCN4CC(c5ccc(Nc6ccccn6)cc5Cl)C4)ccc3C2=O)C(=O)N1] | 546.4 |
| 2-9 | 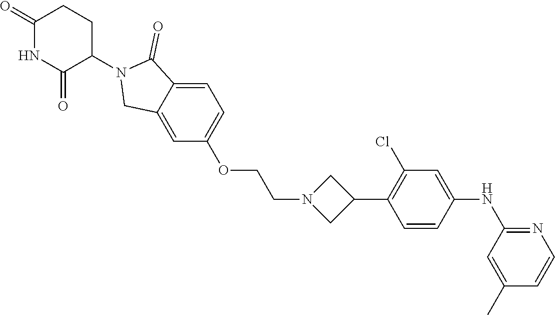 [SMILES: Cc1ccnc(Nc2ccc(C3CN(CCOc4ccc5c(c4)CN(C4CCC(=O)NC4=O)C5=O)C3)c(Cl)c2)c1] | 560.3 |
| 2-10 | 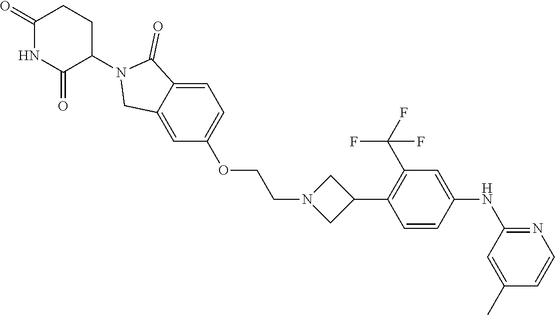 [SMILES: Cc1ccnc(Nc2ccc(C3CN(CCOc4ccc5c(c4)CN(C4CCC(=O)NC4=O)C5=O)C3)c(C(F)(F)F)c2)c1] | 594.4 |
| 2-11 | 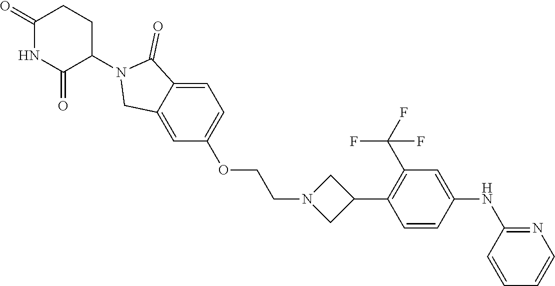 [SMILES: O=C1CCC(N2Cc3cc(OCCN4CC(c5ccc(Nc6ccccn6)cc5C(F)(F)F)C4)ccc3C2=O)C(=O)N1] | 580.3 |
| 2-12 | 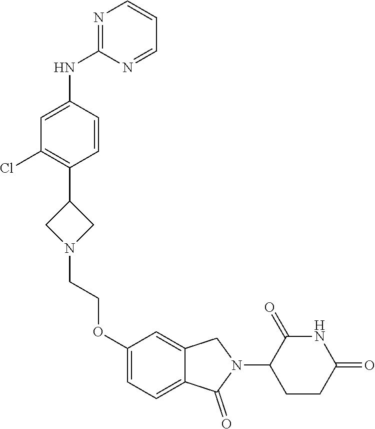 [SMILES: O=C1CCC(N2Cc3cc(OCCN4CC(c5ccc(Nc6ncccn6)cc5Cl)C4)ccc3C2=O)C(=O)N1] | 547.3 |
| 2-13 | 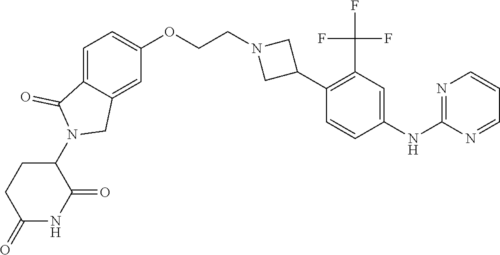 [SMILES: O=C1CCC(N2Cc3cc(OCCN4CC(c5ccc(Nc6ncccn6)cc5C(F)(F)F)C4)ccc3C2=O)C(=O)N1] | 581.4 |
| 2-14 | 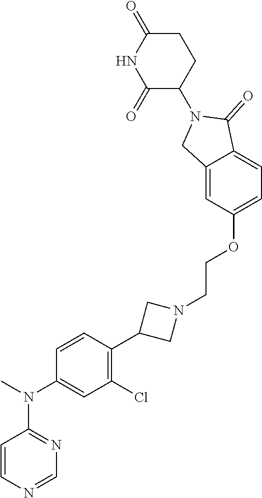 [SMILES: CN(c1ccc(C2CN(CCOc3ccc4c(c3)CN(C3CCC(=O)NC3=O)C4=O)C2)c(Cl)c1)c1ccncn1] | 561.4 |
| 2-15 | 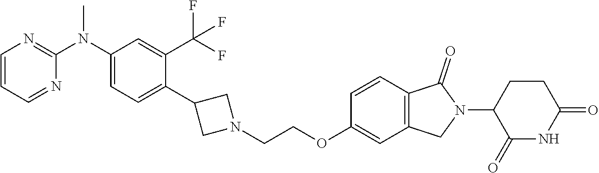 [SMILES: CN(c1ccc(C2CN(CCOc3ccc4c(c3)CN(C3CCC(=O)NC3=O)C4=O)C2)c(C(F)(F)F)c1)c1ncccn1] | 595.3 |
| 2-16 | 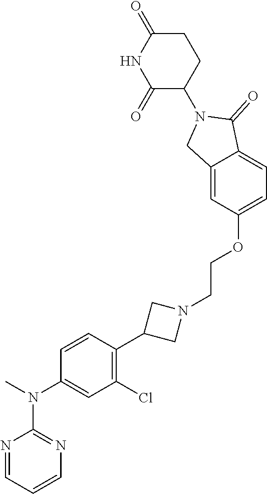 [SMILES: CN(c1ccc(C2CN(CCOc3ccc4c(c3)CN(C3CCC(=O)NC3=O)C4=O)C2)c(Cl)c1)c1ncccn1] | 561.3 |
| 2-17 | 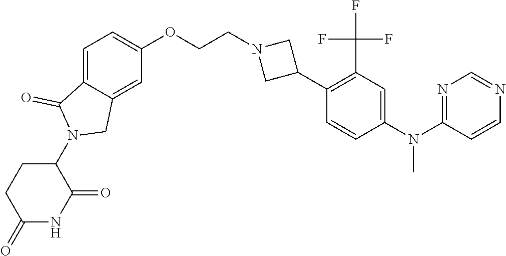 [SMILES: CN(c1ccc(C2CN(CCOc3ccc4c(c3)CN(C3CCC(=O)NC3=O)C4=O)C2)c(C(F)(F)F)c1)c1ccncn1] | 595.4 |
| 2-18 | 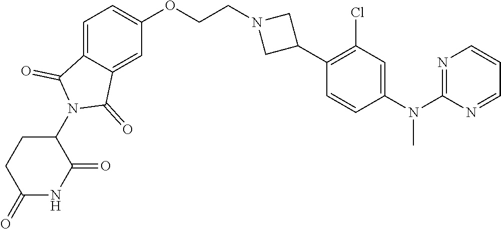 [SMILES: CN(c1ccc(C2CN(CCOc3ccc4c(c3)C(=O)N(C3CCC(=O)NC3=O)C4=O)C2)c(Cl)c1)c1ncccn1] | 575.4 |
| 2-19 | 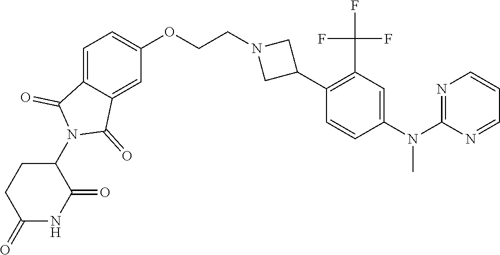 [SMILES: CN(c1ccc(C2CN(CCOc3ccc4c(c3)C(=O)N(C3CCC(=O)NC3=O)C4=O)C2)c(C(F)(F)F)c1)c1ncccn1] | 609.4 |
| 2-20 | 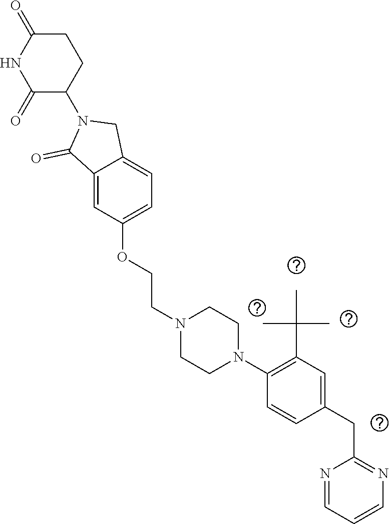 [SMILES: CC(C)(C)c1cc(Cc2ncccn2)ccc1N1CCN(CCOc2ccc3c(c2)C(=O)N(C2CCC(=O)NC2=O)C3)CC1] | 610.5 |
| 2-21 | 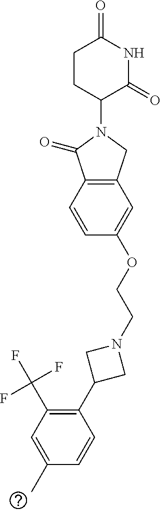 [SMILES: Cc1ccc(C2CN(CCOc3ccc4c(c3)CN(C3CCC(=O)NC3=O)C4=O)C2)c(C(F)(F)F)c1] | 522.3 |
| 2-22 | 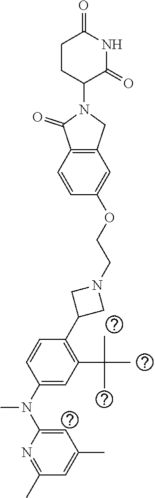 [SMILES: Cc1cc(C)nc(N(C)c2ccc(C3CN(CCOc4ccc5c(c4)CN(C4CCC(=O)NC4=O)C5=O)C3)c(C(C)(C)C)c2)c1] | 623.4 |
| 2-23 | 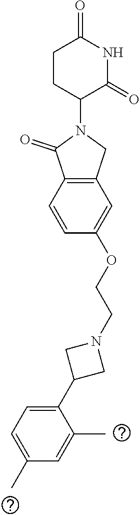 [SMILES: Cc1ccc(C2CN(CCOc3ccc4c(c3)CN(C3CCC(=O)NC3=O)C4=O)C2)c(C)c1] | 488.2 |
| 2-24 | 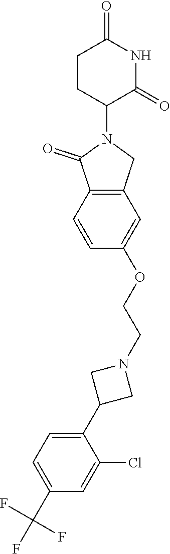 [SMILES: O=C1CCC(N2Cc3cc(OCCN4CC(c5ccc(C(F)(F)F)cc5Cl)C4)ccc3C2=O)C(=O)N1] | 522.3 |
| 2-25 | 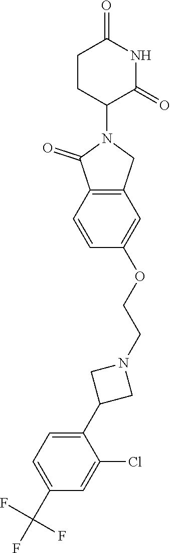 [SMILES: O=C1CCC(N2Cc3cc(OCCN4CC(c5ccc(C(F)(F)F)cc5Cl)C4)ccc3C2=O)C(=O)N1] | 522.3 |
| 2-26 | 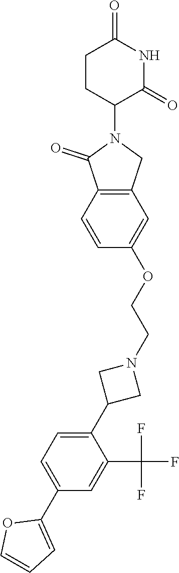 [SMILES: O=C1CCC(N2Cc3cc(OCCN4CC(c5ccc(-c6ccco6)cc5C(F)(F)F)C4)ccc3C2=O)C(=O)N1] | 554.3 |
| 2-27 | 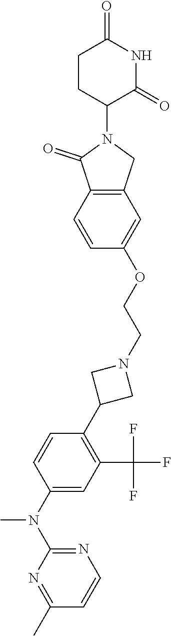 [SMILES: Cc1ccnc(N(C)c2ccc(C3CN(CCOc4ccc5c(c4)CN(C4CCC(=O)NC4=O)C5=O)C3)c(C(F)(F)F)c2)n1] | 609.5 |
| 2-28 | 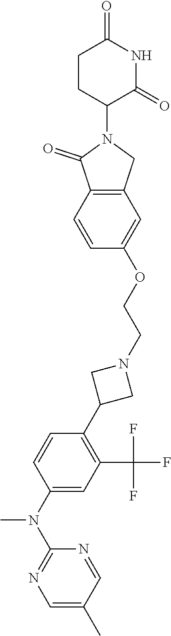 [SMILES: Cc1cnc(N(C)c2ccc(C3CN(CCOc4ccc5c(c4)CN(C4CCC(=O)NC4=O)C5=O)C3)c(C(F)(F)F)c2)nc1] | 609.5 |
| 2-29 | 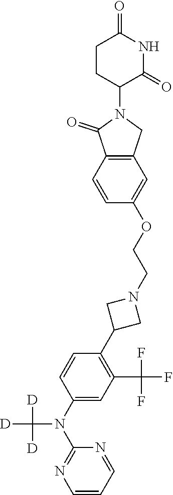 [SMILES: [2H]C([2H])([2H])N(c1ccc(C2CN(CCOc3ccc4c(c3)CN(C3CCC(=O)NC3=O)C4=O)C2)c(C(F)(F)F)c1)c1ncccn1] | 598.5 |
| 2-30 | 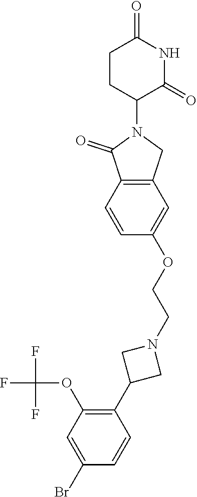 [SMILES: O=C1CCC(N2Cc3cc(OCCN4CC(c5ccc(Br)cc5OC(F)(F)F)C4)ccc3C2=O)C(=O)N1] | 582.3 584.3 |
| 2-31 | 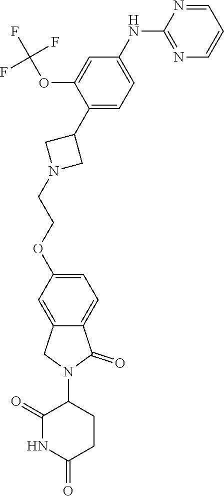 [SMILES: O=C1CCC(N2Cc3cc(OCCN4CC(c5ccc(Nc6ncccn6)cc5OC(F)(F)F)C4)ccc3C2=O)C(=O)N1] | 597.5 |
| 2-32 | 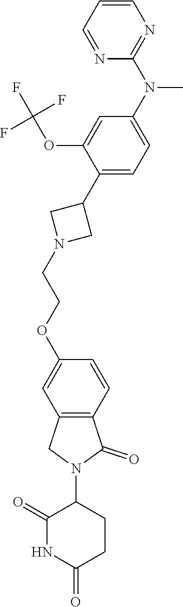 [SMILES: CN(c1ccc(C2CN(CCOc3ccc4c(c3)CN(C3CCC(=O)NC3=O)C4=O)C2)c(OC(F)(F)F)c1)c1ncccn1] | 611.4 |
| 2-33 | 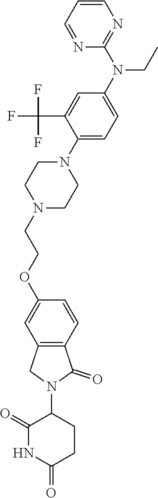 [SMILES: CCN(c1ccc(N2CCN(CCOc3ccc4c(c3)CN(C3CCC(=O)NC3=O)C4=O)CC2)c(C(F)(F)F)c1)c1ncccn1] | 638.4 |
| 2-34 | 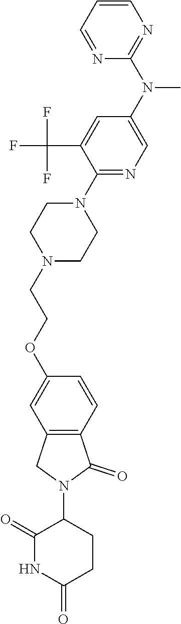 [SMILES: CN(c1cnc(N2CCN(CCOc3ccc4c(c3)CN(C3CCC(=O)NC3=O)C4=O)CC2)c(C(F)(F)F)c1)c1ncccn1] | 625.5 |
| 2-35 | 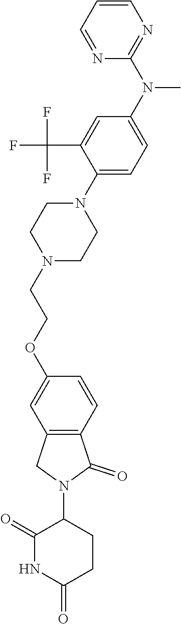 [SMILES: CN(c1ccc(N2CCN(CCOc3ccc4c(c3)CN(C3CCC(=O)NC3=O)C4=O)CC2)c(C(F)(F)F)c1)c1ncccn1] | 624.5 |
| 2-36 | 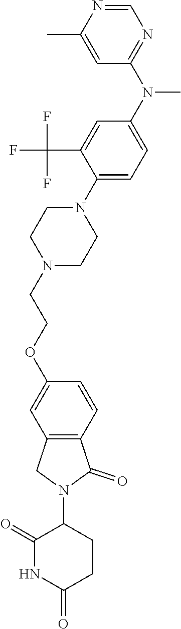 [SMILES: Cc1cc(N(C)c2ccc(N3CCN(CCOc4ccc5c(c4)CN(C4CCC(=O)NC4=O)C5=O)CC3)c(C(F)(F)F)c2)ncn1] | 638.4 |
| 2-37 | 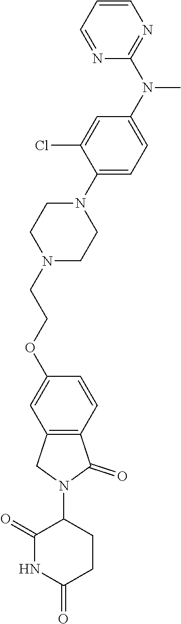 [SMILES: CN(c1ccc(N2CCN(CCOc3ccc4c(c3)CN(C3CCC(=O)NC3=O)C4=O)CC2)c(Cl)c1)c1ncccn1] | 590.4 |
| 2-38 | 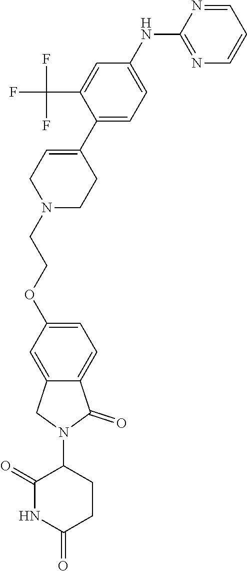 [SMILES: O=C1CCC(N2Cc3cc(OCCN4CC=C(c5ccc(Nc6ncccn6)cc5C(F)(F)F)CC4)ccc3C2=O)C(=O)N1] | 607.4 |
| 2-39 | 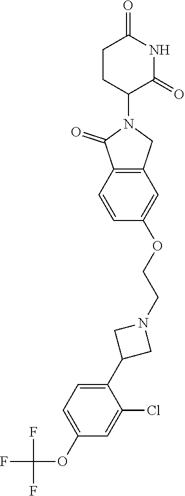 [SMILES: O=C1CCC(N2Cc3cc(OCCN4CC(c5ccc(OC(F)(F)F)cc5Cl)C4)ccc3C2=O)C(=O)N1] | 538.3 |
| 2-40 | 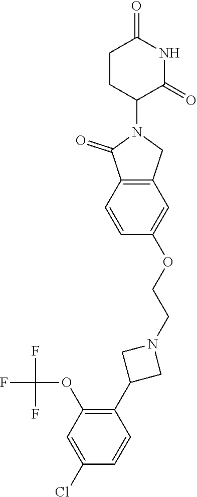 [SMILES: O=C1CCC(N2Cc3cc(OCCN4CC(c5ccc(Cl)cc5OC(F)(F)F)C4)ccc3C2=O)C(=O)N1] | 538.3 |
| 2-41 | 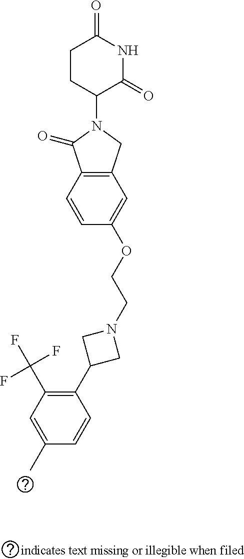 [SMILES: Cc1ccc(C2CN(CCOc3ccc4c(c3)CN(C3CCC(=O)NC3=O)C4=O)C2)c(C(F)(F)F)c1] | 506.3 |
| 2-42 | 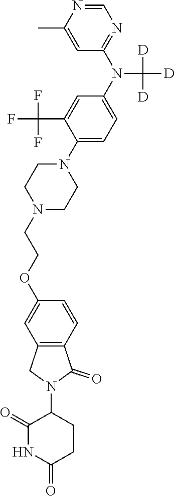 [SMILES: [2H]C([2H])([2H])N(c1ccc(N2CCN(CCOc3ccc4c(c3)CN(C3CCC(=O)NC3=O)C4=O)CC2)c(C(F)(F)F)c1)c1cc(C)ncn1] | 641.6 |
| 2-43 | 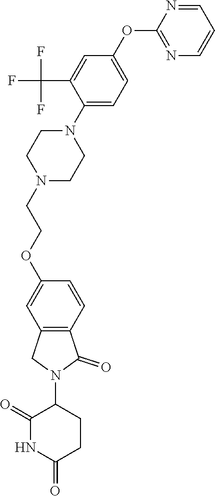 [SMILES: O=C1CCC(N2Cc3cc(OCCN4CCN(c5ccc(Oc6ncccn6)cc5C(F)(F)F)CC4)ccc3C2=O)C(=O)N1] | 611.4 |
| 2-44 | 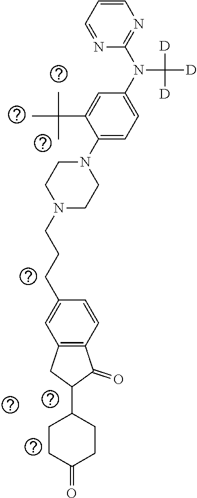 [SMILES: [2H]C([2H])([2H])N(c1ccc(N2CCN(CCCc3ccc4c(c3)CC(C3CCC(=O)CC3)C4=O)CC2)c(C(C)(C)C)c1)c1ncccn1] | 627.5 |
| 2-45 | 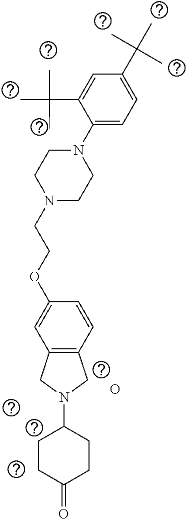 | 585.4 |
| | ¹H NMR (400 MHz, DMSO-d6) δ ppm 1.91-2.05 (m, 1H), 2.31- | |
| | 2.41 (m, 1H), 2.54-2.63 (m, 1H), 2.66 (s, 4H), 2.81 (t, J = 5.6 Hz, | |
| | 2H), 2.94-2.86 (m, 1H), 3.03-2.96 (m, 4H), 4.32-4.16 (m, 3H), | |
| | 4.39 (d, J = 17.2 Hz, 1H), 5.07 (dd, J = 13.2, 5.2 Hz, 1H), 7.11- | |
| | 7.05 (m, 1H), 7.20 (s, 1H), 7.63 (d, J = 8.4 Hz, 1H), 7.69 (d, J = 8.4 | |
| | Hz, 1H), 7.93 (s, 1H), 8.00 (d, J = 8.6 Hz, 1H), 10.97 (s, 1H) | |
| 2-46 | 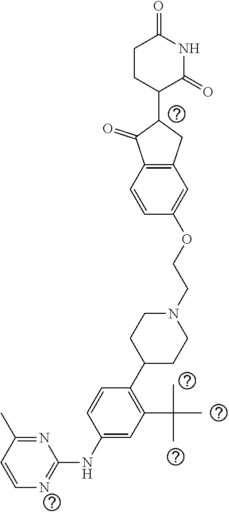 [SMILES: Cc1ccnc(Nc2ccc(C3CCN(CCOc4ccc5c(c4)CC(C4CCC(=O)NC4=O)C5=O)CC3)c(C(C)(C)C)c2)n1] | 623.5 |
| 2-47 | 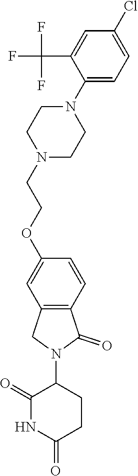 [SMILES: O=C1CCC(N2Cc3cc(OCCN4CCN(c5ccc(Cl)cc5C(F)(F)F)CC4)ccc3C2=O)C(=O)N1] | 551.4 |
| 2-48 | 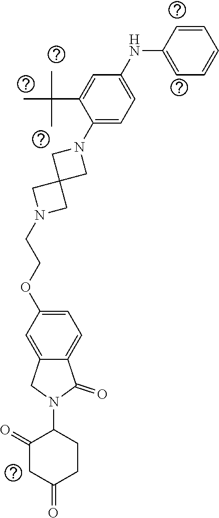 [SMILES: CC(C)(C)c1cc(Nc2ccccc2)ccc1N1CC2(CN(CCOc3ccc4c(c3)CN(C3CCC(=O)CC3=O)C4=O)C2)C1] | 622.4 |
| 2-49 | 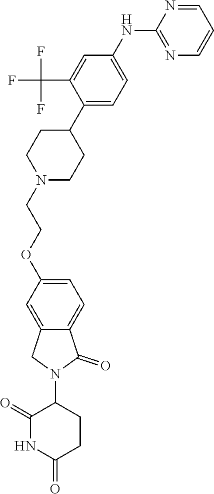 [SMILES: O=C1CCC(N2Cc3cc(OCCN4CCC(c5ccc(Nc6ncccn6)cc5C(F)(F)F)CC4)ccc3C2=O)C(=O)N1] | 609.6 |
| 2-50 | 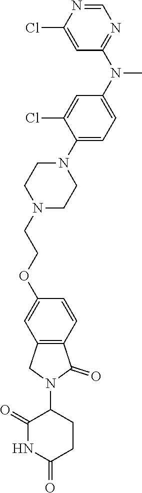 [SMILES: CN(c1ccc(N2CCN(CCOc3ccc4c(c3)CN(C3CCC(=O)NC3=O)C4=O)CC2)c(Cl)c1)c1cc(Cl)ncn1] | 624.4 |
| 2-51 | 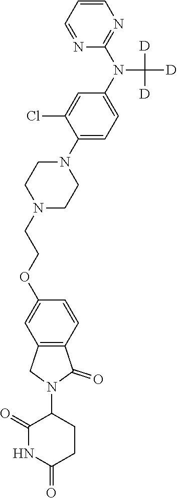 [SMILES: [2H]C([2H])([2H])N(c1ccc(N2CCN(CCOc3ccc4c(c3)CN(C3CCC(=O)NC3=O)C4=O)CC2)c(Cl)c1)c1ncccn1] | 593.5 |
| 2-52 | 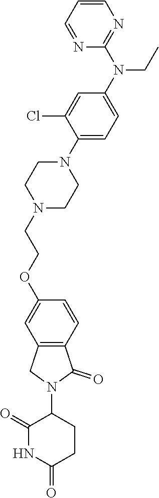 [SMILES: CCN(c1ccc(N2CCN(CCOc3ccc4c(c3)CN(C3CCC(=O)NC3=O)C4=O)CC2)c(Cl)c1)c1ncccn1] | 604.5 |
| 2-53 | 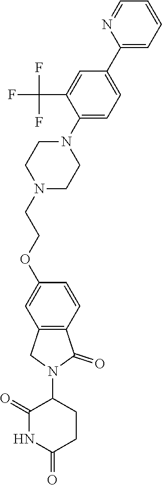 [SMILES: O=C1CCC(N2Cc3cc(OCCN4CCN(c5ccc(-c6ccccn6)cc5C(F)(F)F)CC4)ccc3C2=O)C(=O)N1] | 594.4 |
| 2-54 | 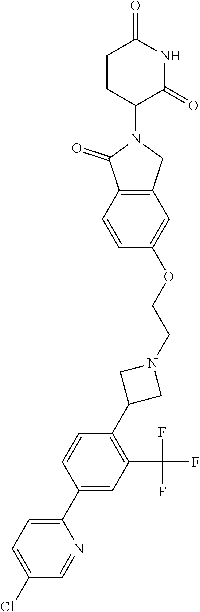 [SMILES: O=C1CCC(N2Cc3cc(OCCN4CC(c5ccc(-c6ccc(Cl)cn6)cc5C(F)(F)F)C4)ccc3C2=O)C(=O)N1] | 599.4 |
| 2-55 | 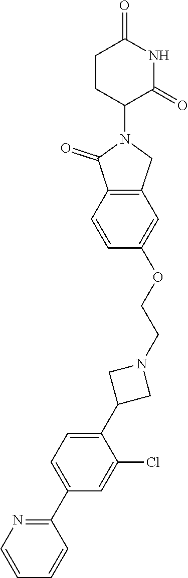 [SMILES: O=C1CCC(N2Cc3cc(OCCN4CC(c5ccc(-c6ccccn6)cc5Cl)C4)ccc3C2=O)C(=O)N1] | 531.4 |
| 2-56 | 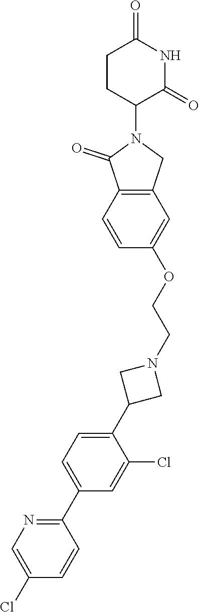 [SMILES: O=C1CCC(N2Cc3cc(OCCN4CC(c5ccc(-c6ccc(Cl)cn6)cc5Cl)C4)ccc3C2=O)C(=O)N1] | 565.3 |
| 2-57 | 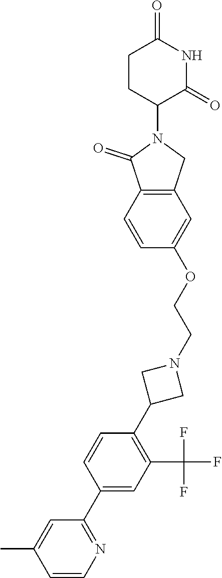 [SMILES: Cc1ccnc(-c2ccc(C3CN(CCOc4ccc5c(c4)CN(C4CCC(=O)NC4=O)C5=O)C3)c(C(F)(F)F)c2)c1] | 579.4 |
| 2-58 | 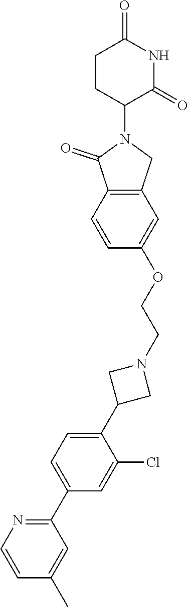 [SMILES: Cc1ccnc(-c2ccc(C3CN(CCOc4ccc5c(c4)CN(C4CCC(=O)NC4=O)C5=O)C3)c(Cl)c2)c1] | 545.4 |
| 2-59 | 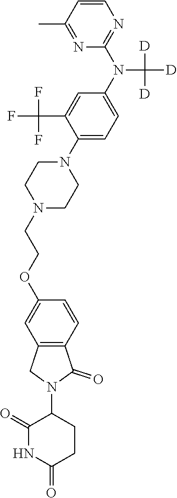 [SMILES: [2H]C([2H])([2H])N(c1ccc(N2CCN(CCOc3ccc4c(c3)CN(C3CCC(=O)NC3=O)C4=O)CC2)c(C(F)(F)F)c1)c1nccc(C)n1] | 641.5 |
| 2-60 | 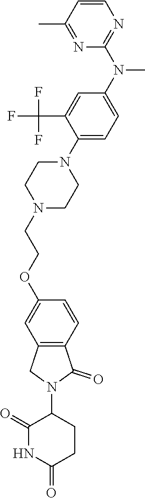 [SMILES: Cc1ccnc(N(C)c2ccc(N3CCN(CCOc4ccc5c(c4)CN(C4CCC(=O)NC4=O)C5=O)CC3)c(C(F)(F)F)c2)n1] | 638.5 |
| 2-61 | 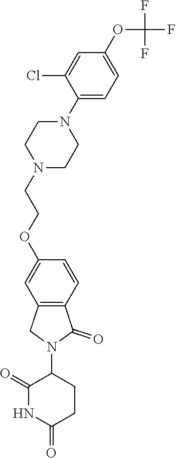 [SMILES: O=C1CCC(N2Cc3cc(OCCN4CCN(c5ccc(OC(F)(F)F)cc5Cl)CC4)ccc3C2=O)C(=O)N1] | 567.4 |
| 2-62 | 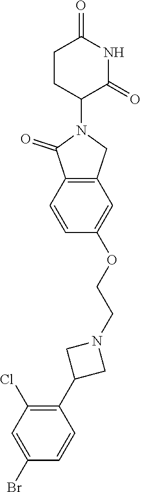 [SMILES: O=C1CCC(N2Cc3cc(OCCN4CC(c5ccc(Br)cc5Cl)C4)ccc3C2=O)C(=O)N1] | 532.2 534.2 |
| 2-63 | 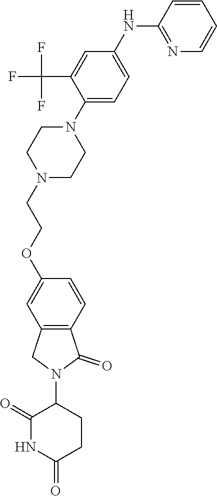 [SMILES: O=C1CCC(N2Cc3cc(OCCN4CCN(c5ccc(Nc6ccccn6)cc5C(F)(F)F)CC4)ccc3C2=O)C(=O)N1] | 609.5 |
| 2-64 | 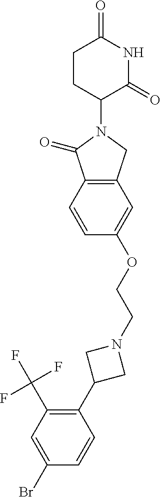 [SMILES: O=C1CCC(N2Cc3cc(OCCN4CC(c5ccc(Br)cc5C(F)(F)F)C4)ccc3C2=O)C(=O)N1] | 566.3 568.3 |
| 2-65 | 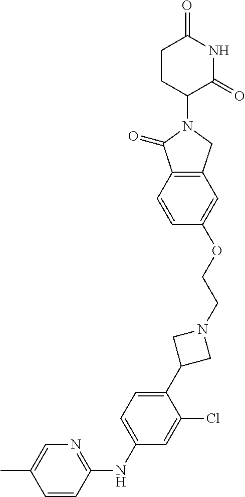 [SMILES: Cc1ccc(Nc2ccc(C3CN(CCOc4ccc5c(c4)CN(C4CCC(=O)NC4=O)C5=O)C3)c(Cl)c2)nc1] | 560.4 |
| 2-66 | 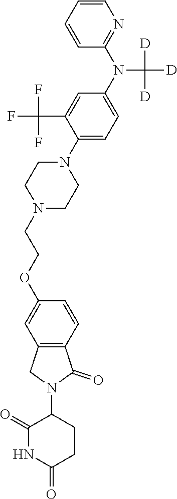 [SMILES: [2H]C([2H])([2H])N(c1ccc(N2CCN(CCOc3ccc4c(c3)CN(C3CCC(=O)NC3=O)C4=O)CC2)c(C(F)(F)F)c1)c1ccccn1] | 626.5 |
| 2-67 | 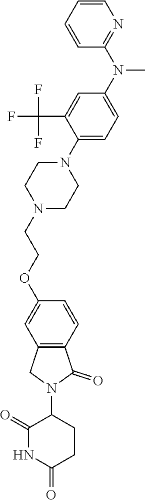 [SMILES: CN(c1ccc(N2CCN(CCOc3ccc4c(c3)CN(C3CCC(=O)NC3=O)C4=O)CC2)c(C(F)(F)F)c1)c1ccccn1] | 623.5 |
| 2-68 | 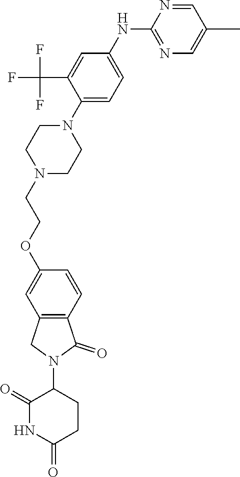 [SMILES: Cc1cnc(Nc2ccc(N3CCN(CCOc4ccc5c(c4)CN(C4CCC(=O)NC4=O)C5=O)CC3)c(C(F)(F)F)c2)nc1] | 624.3 |
| 2-69 | 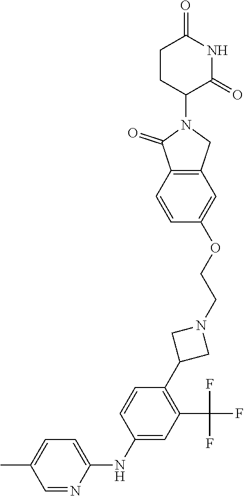 [SMILES: Cc1ccc(Nc2ccc(C3CN(CCOc4ccc5c(c4)CN(C4CCC(=O)NC4=O)C5=O)C3)c(C(F)(F)F)c2)nc1] | 594.3 |
| 2-70 | 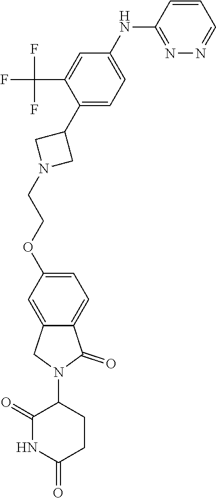 [SMILES: O=C1CCC(N2Cc3cc(OCCN4CC(c5ccc(Nc6cccnn6)cc5C(F)(F)F)C4)ccc3C2=O)C(=O)N1] | 581.2 |
| 2-71 | 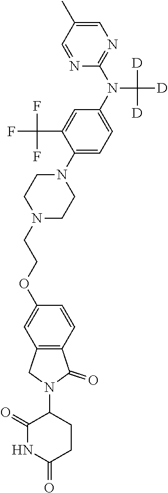 [SMILES: [2H]C([2H])([2H])N(c1ccc(N2CCN(CCOc3ccc4c(c3)CN(C3CCC(=O)NC3=O)C4=O)CC2)c(C(F)(F)F)c1)c1ncc(C)cn1] | 641.4 |
| 2-72 | 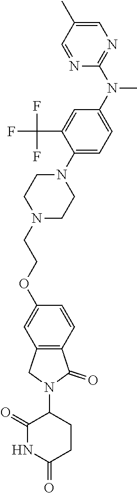 [SMILES: Cc1cnc(N(C)c2ccc(N3CCN(CCOc4ccc5c(c4)CN(C4CCC(=O)NC4=O)C5=O)CC3)c(C(F)(F)F)c2)nc1] | 638.3 |
| 2-73 | 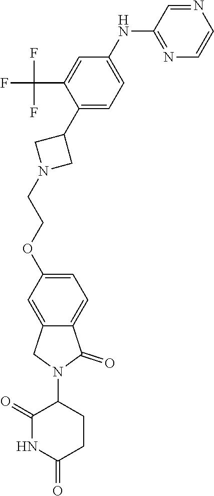 [SMILES: O=C1CCC(N2Cc3cc(OCCN4CC(c5ccc(Nc6cnccn6)cc5C(F)(F)F)C4)ccc3C2=O)C(=O)N1] | 581.2 |
| 2-74 | 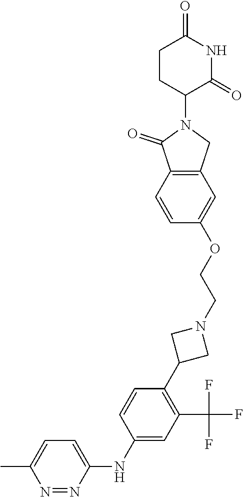 [SMILES: Cc1ccc(Nc2ccc(C3CN(CCOc4ccc5c(c4)CN(C4CCC(=O)NC4=O)C5=O)C3)c(C(F)(F)F)c2)nn1] | 595.3 |
| 2-75 | 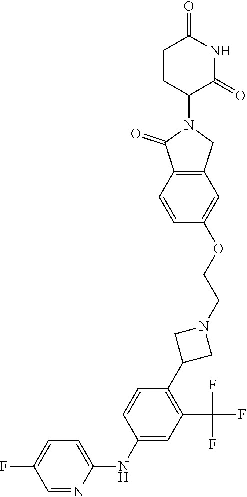 [SMILES: O=C1CCC(N2Cc3cc(OCCN4CC(c5ccc(Nc6ccc(F)cn6)cc5C(F)(F)F)C4)ccc3C2=O)C(=O)N1] | 598.2 |
| 2-76 | 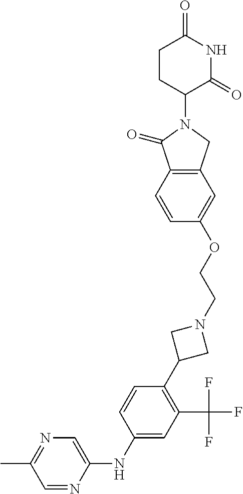 [SMILES: Cc1cnc(Nc2ccc(C3CN(CCOc4ccc5c(c4)CN(C4CCC(=O)NC4=O)C5=O)C3)c(C(F)(F)F)c2)cn1] | 595.2 |
| 2-77 | 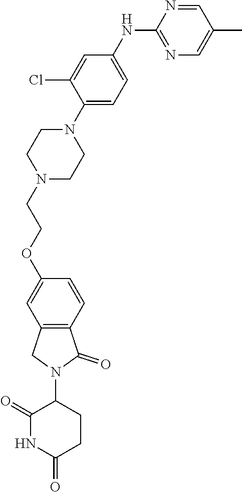 [SMILES: Cc1cnc(Nc2ccc(N3CCN(CCOc4ccc5c(c4)CN(C4CCC(=O)NC4=O)C5=O)CC3)c(Cl)c2)nc1] | 590.3 |
| 2-78 | 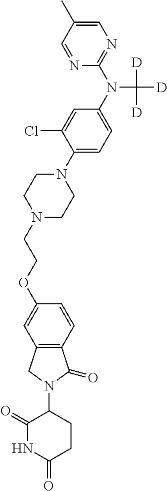 [SMILES: [2H]C([2H])([2H])N(c1ccc(N2CCN(CCOc3ccc4c(c3)CN(C3CCC(=O)NC3=O)C4=O)CC2)c(Cl)c1)c1ncc(C)cn1] | 607.3 |
| 2-79 | 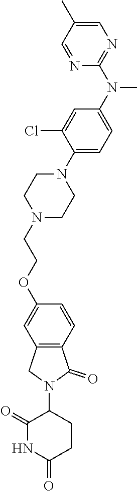 [SMILES: Cc1cnc(N(C)c2ccc(N3CCN(CCOc4ccc5c(c4)CN(C4CCC(=O)NC4=O)C5=O)CC3)c(Cl)c2)nc1] | 604.3 |
| 2-80 | 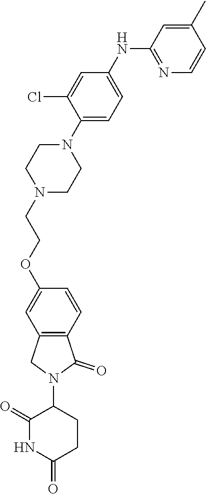 [SMILES: Cc1ccnc(Nc2ccc(N3CCN(CCOc4ccc5c(c4)CN(C4CCC(=O)NC4=O)C5=O)CC3)c(Cl)c2)c1] | 589.3 |
| 2-81 | 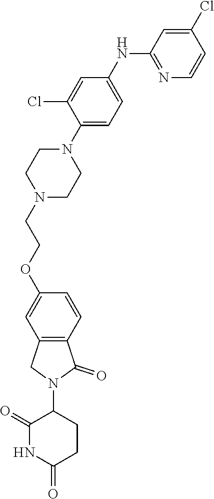 [SMILES: O=C1CCC(N2Cc3cc(OCCN4CCN(c5ccc(Nc6cc(Cl)ccn6)cc5Cl)CC4)ccc3C2=O)C(=O)N1] | 609.1 |
| 2-82 | 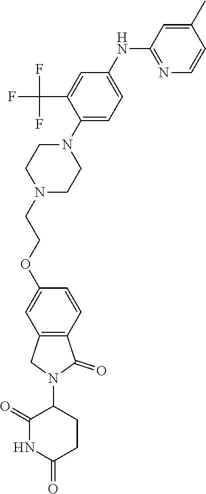 [SMILES: Cc1ccnc(Nc2ccc(N3CCN(CCOc4ccc5c(c4)CN(C4CCC(=O)NC4=O)C5=O)CC3)c(C(F)(F)F)c2)c1] | 623.3 |
| 2-83 | 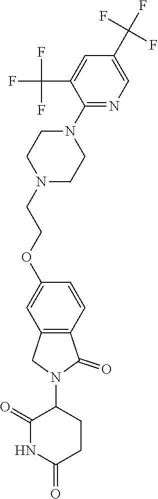 [SMILES: O=C1CCC(N2Cc3cc(OCCN4CCN(c5ncc(C(F)(F)F)cc5C(F)(F)F)CC4)ccc3C2=O)C(=O)N1] | 586.3 |
| 2-84 | 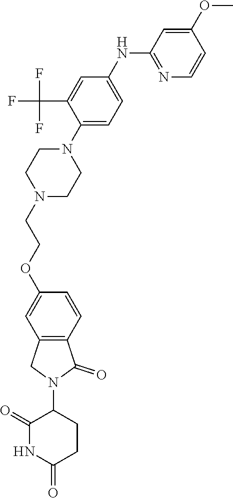 [SMILES: COc1ccnc(Nc2ccc(N3CCN(CCOc4ccc5c(c4)CN(C4CCC(=O)NC4=O)C5=O)CC3)c(C(F)(F)F)c2)c1] | 639.5 |
| 2-85 | 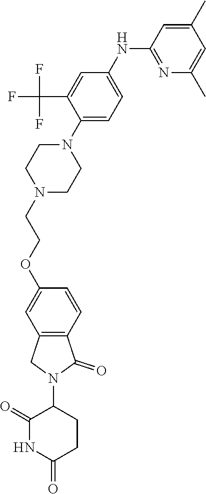 [SMILES: Cc1cc(C)nc(Nc2ccc(N3CCN(CCOc4ccc5c(c4)CN(C4CCC(=O)NC4=O)C5=O)CC3)c(C(F)(F)F)c2)c1] | 637.5 |
| 2-86 | 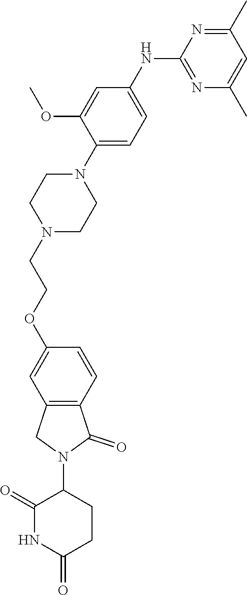 [SMILES: COc1cc(Nc2nc(C)cc(C)n2)ccc1N1CCN(CCOc2ccc3c(c2)CN(C2CCC(=O)NC2=O)C3=O)CC1] | 600.3 |
| 2-87 | 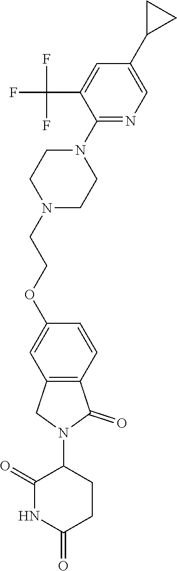 [SMILES: O=C1CCC(N2Cc3cc(OCCN4CCN(c5ncc(C6CC6)cc5C(F)(F)F)CC4)ccc3C2=O)C(=O)N1] | 558.3 |
| 2-88 | 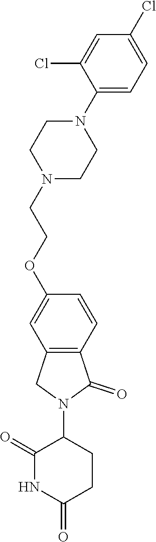 [SMILES: O=C1CCC(N2Cc3cc(OCCN4CCN(c5ccc(Cl)cc5Cl)CC4)ccc3C2=O)C(=O)N1] | 517.2 |
| 2-89 | 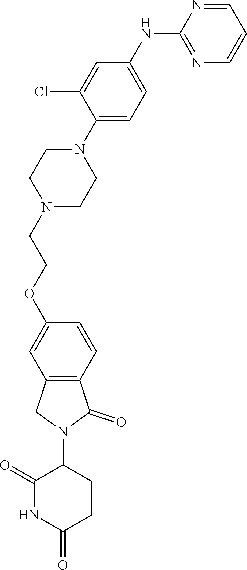 [SMILES: O=C1CCC(N2Cc3cc(OCCN4CCN(c5ccc(Nc6ncccn6)cc5Cl)CC4)ccc3C2=O)C(=O)N1] | 576.3 |
| 2-90 | 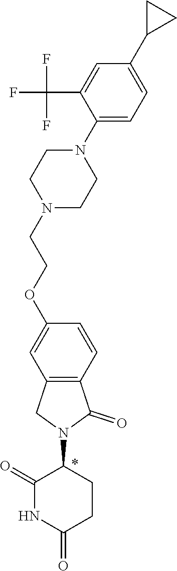 [SMILES: O=C1CC[C@H](N2Cc3cc(OCCN4CCN(c5ccc(C6CC6)cc5C(F)(F)F)CC4)ccc3C2=O)C(=O)N1] | 557.3 |
| 2-91 | 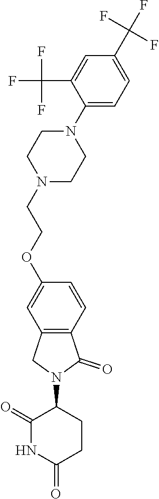 [SMILES: O=C1CC[C@H](N2Cc3cc(OCCN4CCN(c5ccc(C(F)(F)F)cc5C(F)(F)F)CC4)ccc3C2=O)C(=O)N1] | 585.3 |
| 2-92 | 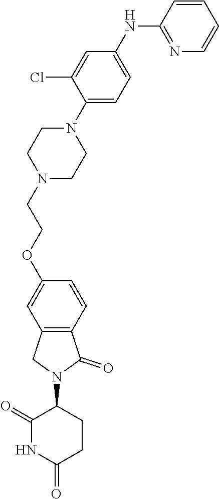 [SMILES: O=C1CC[C@H](N2Cc3cc(OCCN4CCN(c5ccc(Nc6ccccn6)cc5Cl)CC4)ccc3C2=O)C(=O)N1] | 575.3 |
| 2-93 | 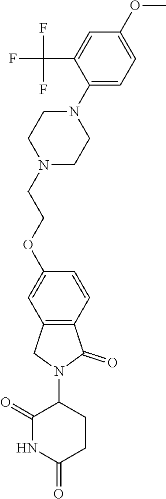 [SMILES: COc1ccc(N2CCN(CCOc3ccc4c(c3)CN(C3CCC(=O)NC3=O)C4=O)CC2)c(C(F)(F)F)c1] | 547.3 |
| 2-94 | 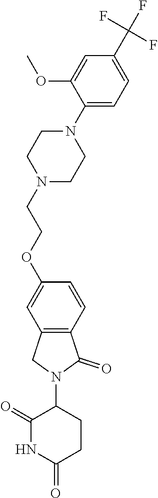 [SMILES: COc1cc(C(F)(F)F)ccc1N1CCN(CCOc2ccc3c(c2)CN(C2CCC(=O)NC2=O)C3=O)CC1] | 547.3 |
| 2-95 | 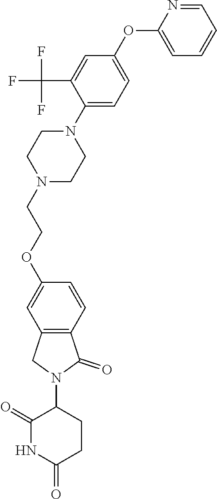 [SMILES: O=C1CCC(N2Cc3cc(OCCN4CCN(c5ccc(Oc6ccccn6)cc5C(F)(F)F)CC4)ccc3C2=O)C(=O)N1] | 610.3 |
| 2-96 | 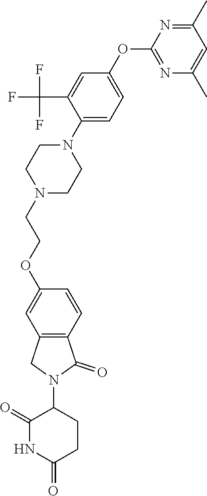 [SMILES: Cc1cc(C)nc(Oc2ccc(N3CCN(CCOc4ccc5c(c4)CN(C4CCC(=O)NC4=O)C5=O)CC3)c(C(F)(F)F)c2)n1] | 639.3 |
| 2-97 | 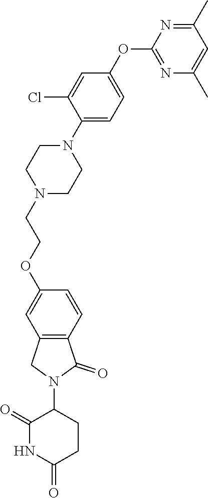 [SMILES: Cc1cc(C)nc(Oc2ccc(N3CCN(CCOc4ccc5c(c4)CN(C4CCC(=O)NC4=O)C5=O)CC3)c(Cl)c2)n1] | 605.3 |
| 2-98 | 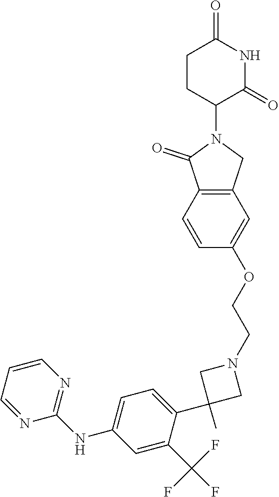 [SMILES: CC1(c2ccc(Nc3ncccn3)cc2C(F)(F)F)CN(CCOc2ccc3c(c2)CN(C2CCC(=O)NC2=O)C3=O)C1] | 595.4 |
| 2-99 | 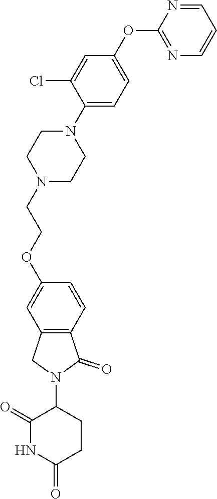 [SMILES: O=C1CCC(N2Cc3cc(OCCN4CCN(c5ccc(Oc6ncccn6)cc5Cl)CC4)ccc3C2=O)C(=O)N1] | 577.2 |
| 2-100 | 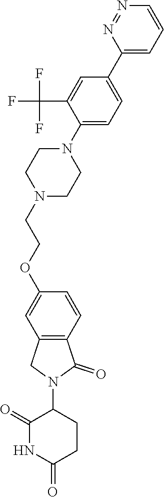 [SMILES: O=C1CCC(N2Cc3cc(OCCN4CCN(c5ccc(-c6cccnn6)cc5C(F)(F)F)CC4)ccc3C2=O)C(=O)N1] | 575.5 |
| 2-101 | 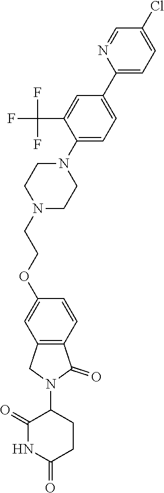 [SMILES: O=C1CCC(N2Cc3cc(OCCN4CCN(c5ccc(-c6ccc(Cl)cn6)cc5C(F)(F)F)CC4)ccc3C2=O)C(=O)N1] | 628.4 |
| 2-102 | 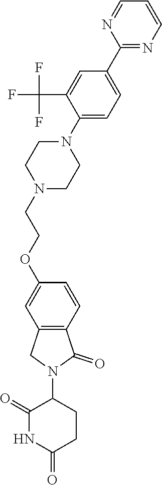 [SMILES: O=C1CCC(N2Cc3cc(OCCN4CCN(c5ccc(-c6ncccn6)cc5C(F)(F)F)CC4)ccc3C2=O)C(=O)N1] | 575.4 |
| 2-103 | 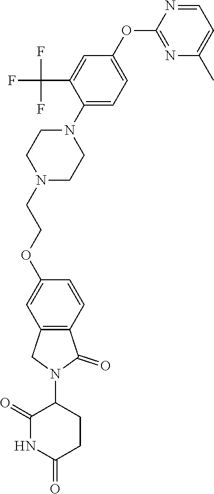 [SMILES: Cc1ccnc(Oc2ccc(N3CCN(CCOc4ccc5c(c4)CN(C4CCC(=O)NC4=O)C5=O)CC3)c(C(F)(F)F)c2)n1] | 625.5 |
| 2-104 | 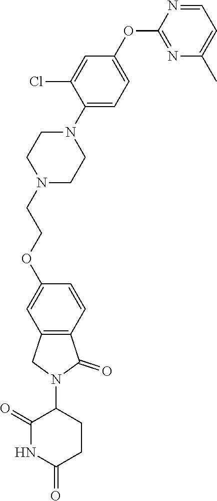 [SMILES: Cc1ccnc(Oc2ccc(N3CCN(CCOc4ccc5c(c4)CN(C4CCC(=O)NC4=O)C5=O)CC3)c(Cl)c2)n1] | 591.1 |
| | ¹H NMR (400 MHz, DMSO-d6) δ ppm 1.94-2.06(m, 1H), 2.34- | |
| | 2.37 (m, 1H), 2.42 (s, 3H), 2.54-2.57 (m, 1H), 2.70 (s, 4H), 2.81 (t, | |
| | J = 5.4 Hz, 2H), 2.85-2.94 (m, 1H), 2.97-3.06 (m, 4H), 4.32-4.18 | |
| | (m, 3H), 4.45-4.27 (m, 1H), 5.07 (dd, J = 13.2, 4.8 Hz, 1H), 7.06- | |
| | 7.10 (m, 1H), 7.11-7.17 (m, 2H), 7.19-7.24 (m, 2H), 7.31 (d, J = 2.4 | |
| | Hz, 1H), 7.64 (d, J = 8.6 Hz, 1H), 8.46 (d, J = 5.0 Hz, 1H), 10.96 (s, | |
| | 1H) | |
| 2-105 | 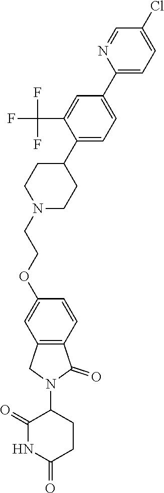 [SMILES: O=C1CCC(N2Cc3cc(OCCN4CCC(c5ccc(-c6ccc(Cl)cn6)cc5C(F)(F)F)CC4)ccc3C2=O)C(=O)N1] | 627.4 |
| 2-106 | 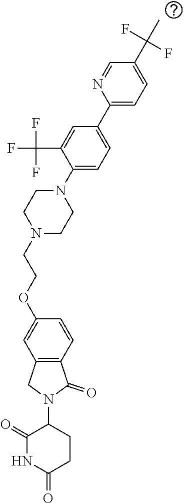 [SMILES: CC(F)(F)c1ccc(-c2ccc(N3CCN(CCOc4ccc5c(c4)CN(C4CCC(=O)NC4=O)C5=O)CC3)c(C(F)(F)F)c2)nc1] | 661.4 |
| 2-107 | 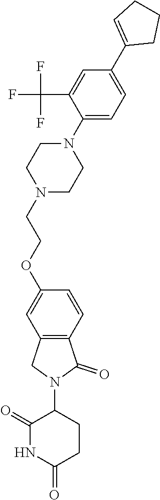 [SMILES: O=C1CCC(N2Cc3cc(OCCN4CCN(c5ccc(C6=CCCC6)cc5C(F)(F)F)CC4)ccc3C2=O)C(=O)N1] | 583.4 |
| 2-108 | 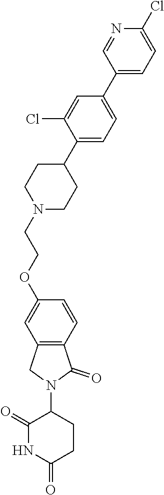 [SMILES: O=C1CCC(N2Cc3cc(OCCN4CCC(c5ccc(-c6ccc(Cl)nc6)cc5Cl)CC4)ccc3C2=O)C(=O)N1] | 593.4 |
| 2-109 | 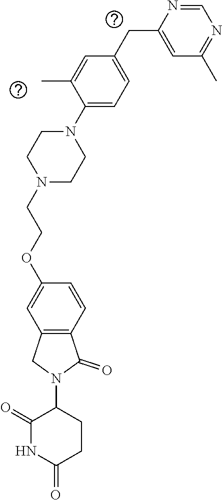 [SMILES: Cc1cc(Cc2ccc(N3CCN(CCOc4ccc5c(c4)CN(C4CCC(=O)NC4=O)C5=O)CC3)c(C)c2)ncn1] | 591.4 |
| | ¹H NMR (400 MHz, DMSO-d6) δ ppm 1.92-2.00 (m, 1H), 2.33- | |
| | 2.38 (m, 1H), 2.44 (s, 3H), 2.55-2.64 (m, 1H), 2.76-2.67(m, 4H), | |
| | 2.85 (s, 2H), 2.96-2.88 (m, 1H), 3.01 (s, 4H), 4.31-4.16 (m, 3H), | |
| | 4.40 (d, J = 17.2 Hz, 1H), 5.08 (dd, J = 13.4, 5.2 Hz, 1H), 7.01 (s, | |
| | 1H), 7.09 (d, J = 8.6 Hz, 1H), 7.15 (dd, J = 8.8, 2.6 Hz, 1H), 7.23 | |
| | (d, J = 8.8 Hz, 2H), 7.34 (d, J = 2.6 Hz, 1H), 7.63 (d, J = 8.4 Hz, | |
| | 1H), 8.62 (s, 1H), 10.97 (s, 1H) | |
| 2-110 | 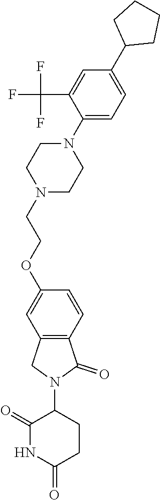 [SMILES: O=C1CCC(N2Cc3cc(OCCN4CCN(c5ccc(C6CCCC6)cc5C(F)(F)F)CC4)ccc3C2=O)C(=O)N1] | 585.4 |
| 2-111 | 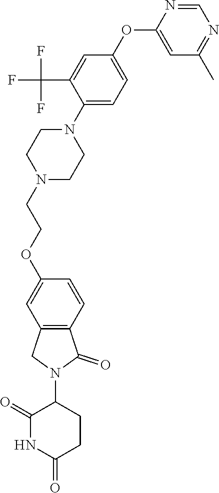 [SMILES: Cc1cc(Oc2ccc(N3CCN(CCOc4ccc5c(c4)CN(C4CCC(=O)NC4=O)C5=O)CC3)c(C(F)(F)F)c2)ncn1] | 625.4 |
| 2-112 | 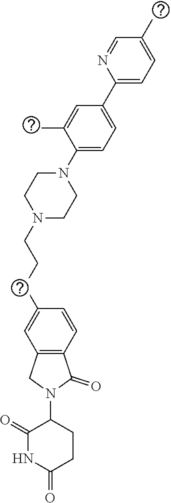 [SMILES: Cc1ccc(-c2ccc(N3CCN(CCCc4ccc5c(c4)CN(C4CCC(=O)NC4=O)C5=O)CC3)c(C)c2)nc1] | 594.3 |
| 2-113 | 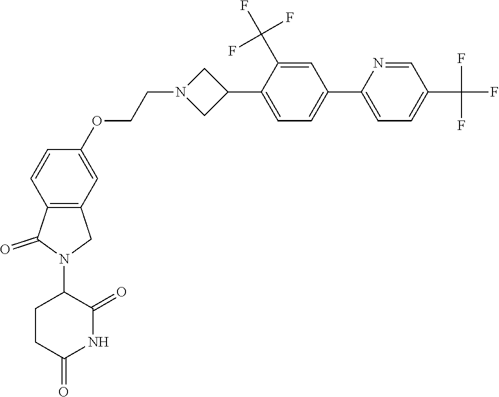 [SMILES: O=C1CCC(N2Cc3cc(OCCN4CC(c5ccc(-c6ccc(C(F)(F)F)cn6)cc5C(F)(F)F)C4)ccc3C2=O)C(=O)N1] | 633.4 |
| 2-114 | 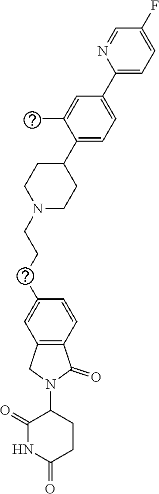 [SMILES: Cc1cc(-c2ccc(F)cn2)ccc1C1CCN(CCCc2ccc3c(c2)CN(C2CCC(=O)NC2=O)C3=O)CC1] | 577.4 |
| 2-115 | 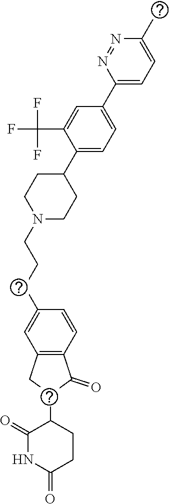 [SMILES: Cc1ccc(-c2ccc(C3CCN(CCCc4ccc5c(c4)CC(C4CCC(=O)NC4=O)C5=O)CC3)c(C(F)(F)F)c2)nn1] | 628.4 |
| 2-116 | 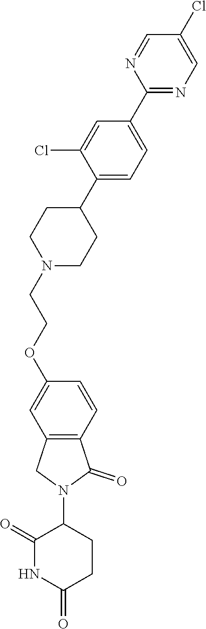 [SMILES: O=C1CCC(N2Cc3cc(OCCN4CCC(c5ccc(-c6ncc(Cl)cn6)cc5Cl)CC4)ccc3C2=O)C(=O)N1] | 594.3 |
| 2-117 | 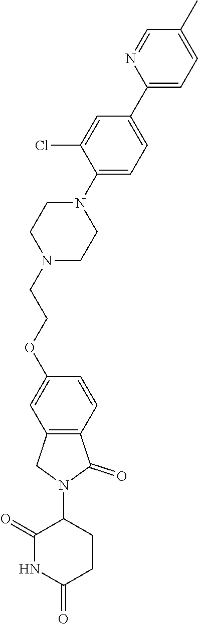 [SMILES: Cc1ccc(-c2ccc(N3CCN(CCOc4ccc5c(c4)CN(C4CCC(=O)NC4=O)C5=O)CC3)c(Cl)c2)nc1] | 574.4 |
| 2-118 | 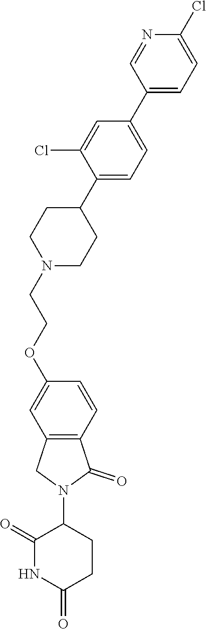 [SMILES: O=C1CCC(N2Cc3cc(OCCN4CCC(c5ccc(-c6ccc(Cl)nc6)cc5Cl)CC4)ccc3C2=O)C(=O)N1] | 593.4 |
| 2-119 | 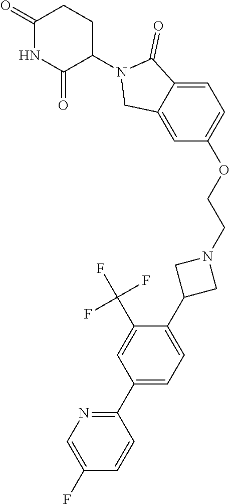 [SMILES: O=C1CCC(N2Cc3cc(OCCN4CC(c5ccc(-c6ccc(F)cn6)cc5C(F)(F)F)C4)ccc3C2=O)C(=O)N1] | 582.5 |
| 2-120 | 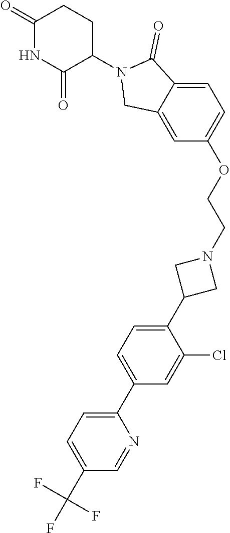 [SMILES: O=C1CCC(N2Cc3cc(OCCN4CC(c5ccc(-c6ccc(C(F)(F)F)cn6)cc5Cl)C4)ccc3C2=O)C(=O)N1] | 599.3 |
| 2-121 | 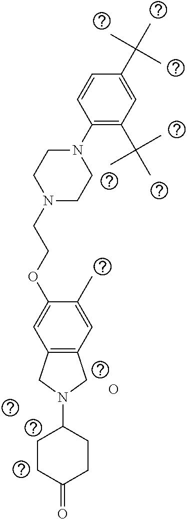 | 603.2 |
| | ¹H NMR (400 MHz, DMSO-d6) δ ppm 1.94-2.05 (m, 1H), 2.25- | |
| | 2.40 (m, 1H), 2.53-2.82 (m, 5H), 2.84-3.11 (m, 5H), 3.34 (br s, | |
| | 2H), 4.32 (d, J = 17.2 Hz, 1H), 4.46 (d, J = 17.6 Hz, 1H), 5.10 (dd, | |
| | J = 13.6, 5.2 Hz, 1H), 7.65 (d, J = 9.6 Hz, 1H), 7.73 (d, J = 8.8 Hz, | |
| | 1H), 7.95 (br s, 1H), 8.03 (d, J = 8.4 Hz, 1H), 8.39 (d, J = 6.6 Hz, | |
| | 1H), 9.94 (br s, 1H), 11.00 (br s, 1H) | |
| 2-122 | 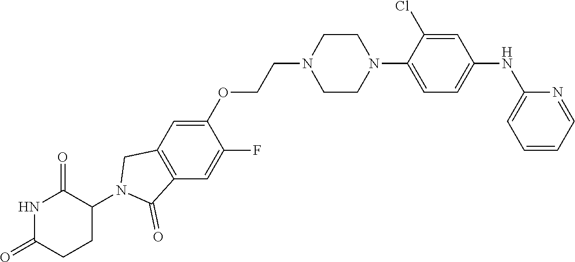 [SMILES: O=C1CCC(N2Cc3cc(OCCN4CCN(c5ccc(Nc6ccccn6)cc5Cl)CC4)c(F)cc3C2=O)C(=O)N1] | 594.2 |
| | ¹H NMR (400 MHz, DMSO-d6) δ ppm 1.97-2.06 (m, 1H), 2.27- | |
| | 2.42 (m, 1H), 2.54-2.81 (m, 5H), 2.83-3.07 (m, 5H), 3.30 (br s, | |
| | 2H), 4.32 (d, J = 17.2 Hz, 1H), 4.46 (d, J = 17.6 Hz, 1H), 5.10 (dd, | |
| | J = 13.2, 5.0 Hz, 1H), 6.67-6.81 (m, 2H), 7.14 (d, J = 8.8 Hz, 1H), | |
| | 7.44-7.67 (m, 3H), 7.98 (d, J = 2.4 Hz, 1H), 8.15 (d, J = 4.2 Hz, | |
| | 1H), 8.41 (d, J = 6.4 Hz, 1H), 9.07 (br s, 1H), 9.96 (br s, 1H), 11.00 | |
| | (br s, 1H) | |
| 2-123 | 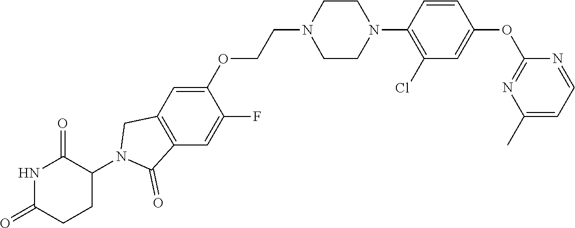 [SMILES: Cc1ccnc(Oc2ccc(N3CCN(CCOc4cc5c(cc4F)C(=O)N(C4CCC(=O)NC4=O)C5)CC3)c(Cl)c2)n1] | 609.2 |
| 2-124 | 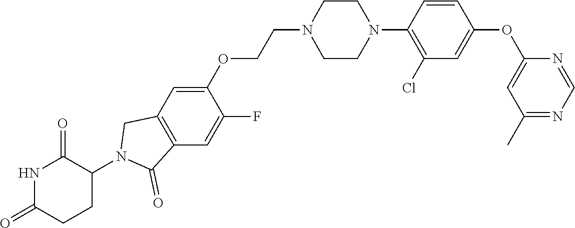 [SMILES: Cc1cc(Oc2ccc(N3CCN(CCOc4cc5c(cc4F)C(=O)N(C4CCC(=O)NC4=O)C5)CC3)c(Cl)c2)ncn1] | 609.0 |
| | ¹H NMR (400 MHz, DMSO-d6) δ ppm 1.94-2.04 (m, 2H), 2.30- | |
| | 2.35 (m, 1H), 2.44 (s, 3H), 2.55-2.62 (m, 1H), 2.69 (s, 4H), 2.86- | |
| | 2.91 (m, 2H), 2.92-3.08 (m, 4H), 4.25-4.32 (m, 3H), 4.40 (d, J = | |
| | 17.4 Hz, 1H), 5.09 (dd, J = 13.3, 4.9 Hz, 1H), 7.01 (s, 1H), 7.15 | |
| | (dd, J = 8.7, 2.6 Hz, 1H), 7.23 (d, J = 8.8 Hz, 1H), 7.34 (d, J = 2.5 | |
| | Hz, 1H), 7.51 (dd, J = 15.7, 8.6 Hz, 2H), 8.62 (s, 1H), 10.98 (s, 1H) | |
| 2-125 | 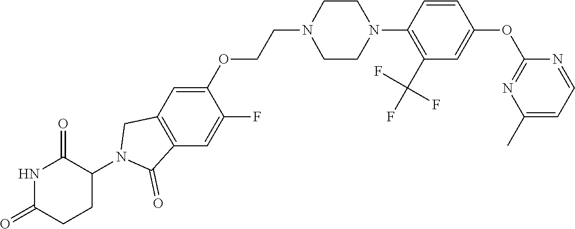 [SMILES: Cc1ccnc(Oc2ccc(N3CCN(CCOc4cc5c(cc4F)C(=O)N(C4CCC(=O)NC4=O)C5)CC3)c(C(F)(F)F)c2)n1] | 643.1 |
| 2-126 | 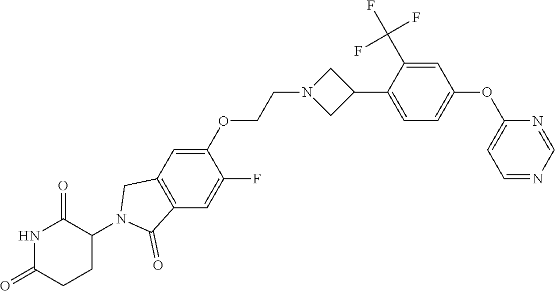 [SMILES: O=C1CCC(N2Cc3cc(OCCN4CC(c5ccc(Oc6ccncn6)cc5C(F)(F)F)C4)c(F)cc3C2=O)C(=O)N1] | 600.2 |
| 2-127 | 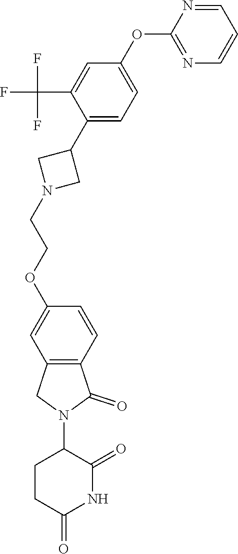 [SMILES: O=C1CCC(N2Cc3cc(OCCN4CC(c5ccc(Oc6ncccn6)cc5C(F)(F)F)C4)ccc3C2=O)C(=O)N1] | 582.2 |
| 2-128 | 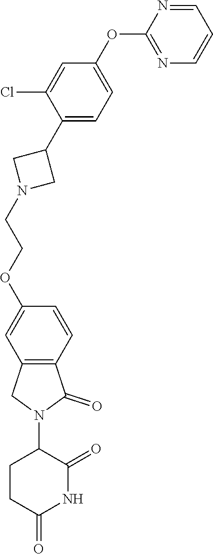 [SMILES: O=C1CCC(N2Cc3cc(OCCN4CC(c5ccc(Oc6ncccn6)cc5Cl)C4)ccc3C2=O)C(=O)N1] | 548.3 |
| 2-129 | 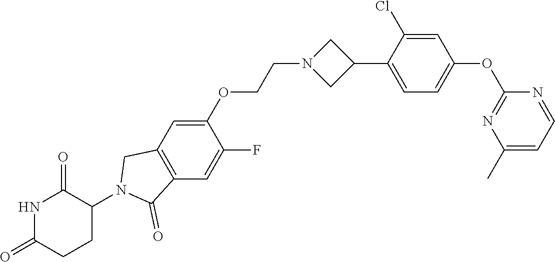 [SMILES: Cc1ccnc(Oc2ccc(C3CN(CCOc4cc5c(cc4F)C(=O)N(C4CCC(=O)NC4=O)C5)C3)c(Cl)c2)n1] | 580.2 |
| 2-130 | 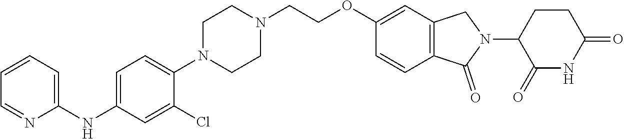 [SMILES: O=C1CCC(N2Cc3cc(OCCN4CCN(c5ccc(Nc6ccccn6)cc5Cl)CC4)ccc3C2=O)C(=O)N1] | 575.3 |
| | ¹H NMR (400 MHz, DMSO-d6) δ ppm 1.90-2.03 (m, 1H), 2.32- | |
| | 2.39 (m, 1H), 2.58-2.70 (m, 5H), 2.78-2.83 (m, 2H), 2.91-2.96 | |
| | (m, 5H), 4.19-4.30 (m, 3H), 4.37-4.44 (m, 2H), 5.08 (dd, J = | |
| | 13.4, 5.4 Hz, 1H), 6.73-6.80 (m, 2H), 7.07-7.13 (m, 2H), 7.21 (br | |
| | s, 1H), 7.46 (d, J = 8.8 Hz, 1H), 7.53-7.58 (m, 1H), 7.63 (d, J = 8.6 | |
| | Hz, 1H), 7.96 (d, J = 2.8 Hz, 1H), 8.14 (d, J = 4.8 Hz, 1H), 9.05 (br | |
| | s, 1H), 10.97 (br s, 1H) | |
| 2-131 | 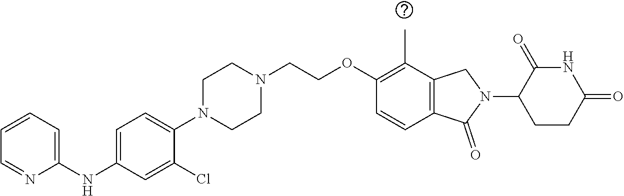 [SMILES: Cc1c(OCCN2CCN(c3ccc(Nc4ccccn4)cc3Cl)CC2)ccc2c1CN(C1CCC(=O)NC1=O)C2=O] | 594.2 |
| | ¹H NMR (400 MHz, DMSO-d6) δ ppm 1.90-2.06 (m, 1H), 2.28- | |
| | 2.39(m, 1H), 2.55-2.70 (m, 1H), 2.82-2.98 (m, 1H), 3.00-3.13 (m, | |
| | 2H), 3.38 (d, J = 10.6 Hz, 4H), 3.81-3.62(m, 4H), 4.38-4.42 (m, | |
| | 1H), 4.52-4.62 (m, 3H), 5.10 (dd, J = 13.4, 5.0 Hz, 1H), 6.74- | |
| | 6.84(m, 2H), 7.18 (d, J = 8.8 Hz, 1H), 7.41-7.54 (m, 2H), 7.56-7.66 | |
| | (m, 2H), 8.00 (d, J = 2.2 Hz, 1H), 8.14 (d, J = 4.2 Hz, 1H), 9.22 (s, | |
| | 1H), 9.90 (s, 1H), 11.00 (s, 1H) | |
| 2-132 | 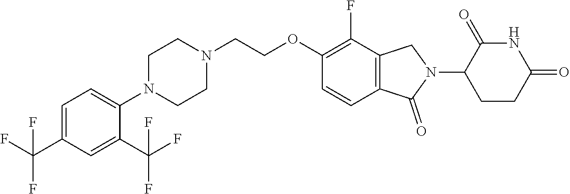 [SMILES: O=C1CCC(N2Cc3c(ccc(OCCN4CCN(c5ccc(C(F)(F)F)cc5C(F)(F)F)CC4)c3F)C2=O)C(=O)N1] | 603.3 |
| 2-133 | 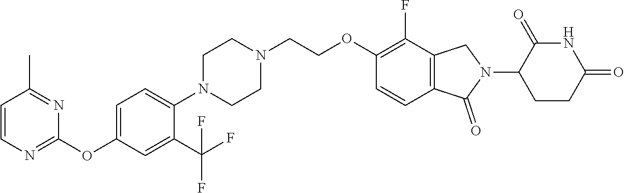 [SMILES: Cc1ccnc(Oc2ccc(N3CCN(CCOc4ccc5c(c4F)CN(C4CCC(=O)NC4=O)C5=O)CC3)c(C(F)(F)F)c2)n1] | 643.2 |
| | ¹H NMR (400 MHz, DMSO-d6) δ ppm 1.94-2.04 (m, 1H), 2.32- | |
| | 2.41 (m, 1H), 2.42 (s, 3H), 2.55-2.69 (m, 1H), 2.83-3.00 (m, 1H), | |
| | 3.18 (s, 4H), 3.50-3.81 (m, 6H), 4.32-4.38(m, 1H), 4.47-4.65 (m, | |
| | 3H), 5.10 (dd, J = 13.4, 5.0 Hz, 1H), 7.19 (d, J = 5.0 Hz, 1H), 7.45 | |
| | (t, J = 7.8 Hz, 1H), 7.50-7.58(m, 2H), 7.59-7.70 (m, 2H), 8.47 (d, | |
| | J = 5.0 Hz, 1H), 10.02 (s, 1H), 11.00 (s, 1H) | |
| 2-134 | 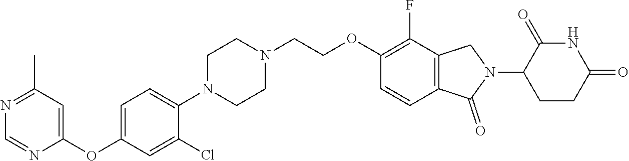 [SMILES: Cc1cc(Oc2ccc(N3CCN(CCOc4ccc5c(c4F)CN(C4CCC(=O)NC4=O)C5=O)CC3)c(Cl)c2)ncn1] | 609.2 |
| | ¹H NMR (400 MHz, DMSO-d6) δ ppm 1.96-2.04 (m, 1H), 2.30- | |
| | 2.35 (m, 1H), 2.45 (s, 3H), 2.56-2.71 (m, 1H), 2.87-3.02 (m, 1H), | |
| | 3.06-3.21 (m, 2H), 3.27-3.60 (m, 8H), 4.38 (d, J = 17.2 Hz, 1H), | |
| | 4.68-4.50 (m, 3H), 5.10 (dd, J = 13.4, 5.2 Hz, 1H), 7.05 (s, 1H), | |
| | 7.21 (d, J = 8.8 Hz, 1H), 7.31 (d, J = 8.8 Hz, 1H), 7.49-7.40 (m, | |
| | 2H), 7.62 (d, J = 8.4 Hz, 1H), 8.62 (s, 1H), 9.97 (s, 1H), 11.01 (s, | |
| | 1H) | |
| 2-135 | 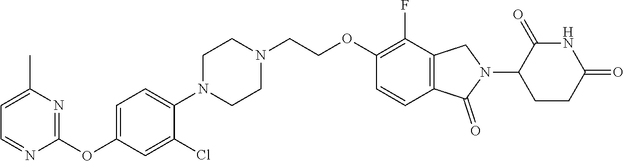 [SMILES: Cc1ccnc(Oc2ccc(N3CCN(CCOc4ccc5c(c4F)CN(C4CCC(=O)NC4=O)C5=O)CC3)c(Cl)c2)n1] | 609.3 |
| | ¹H NMR (400 MHz, DMSO-d6) δ ppm 1.96-2.03 (m, 1H), 2.33 | |
| | (s, 1H), 2.40 (s, 3H), 2.61 (s, 1H), 2.67-2.70 (m, 4H), 2.86 (t, J = | |
| | 5.6 Hz, 2H), 2.91 (s, 1H), 2.99 (s, 4H), 4.25-4.32 (m, 3H), 4.40 | |
| | (d, J = 17.2 Hz, 1H), 5.09 (dd, J = 13.2, 5.2 Hz, 1H), 7.11-7.15 | |
| | (m, 2H), 7.21 (d, J = 8.8 Hz, 1H), 7.31 (d, J = 2.8 Hz, 1H), 7.48- | |
| | 7.54 (m, 2H), 8.45 (d, J = 4.8 Hz, 1H), 10.98 (s, 1H) | |
| 2-136 | 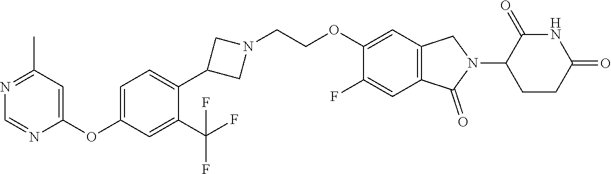 [SMILES: Cc1cc(Oc2ccc(C3CN(CCOc4cc5c(cc4F)C(=O)N(C4CCC(=O)NC4=O)C5)C3)c(C(F)(F)F)c2)ncn1] | 614.3 |
| | ¹H NMR (400 MHz, DMSO-d6) δ ppm 1.97-2.03 (m, 1H), 2.40- | |
| | 2.43 (m, 1H), 2.48 (s, 3H), 2.58-2.62 (m, 1H), 2.87-2.95 (m, 1H), | |
| | 3.81 (s, 2H), 4.30 (d, J = 17.6 Hz, 1H), 4.39-4.44 (m, 4H), 4.50- | |
| | 4.52 (m, 4H), 5.10 (dd, J = 13.2, 5.2 Hz, 1H), 7.14 (s, 1H), 7.50 (d, | |
| | J = 7.2 Hz, 1H), 7.61 (d, J = 10.0 Hz, 1H), 7.65-7.68 (m, 2H), 7.87 | |
| | (d, J = 8.0 Hz, 1H), 8.62 (s, 1H), 10.99 (s, 1H). | |
| 2-137 | 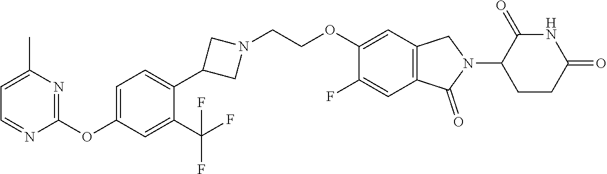 [SMILES: Cc1ccnc(Oc2ccc(C3CN(CCOc4cc5c(cc4F)C(=O)N(C4CCC(=O)NC4=O)C5)C3)c(C(F)(F)F)c2)n1] | 614.3 |
| 2-138 | 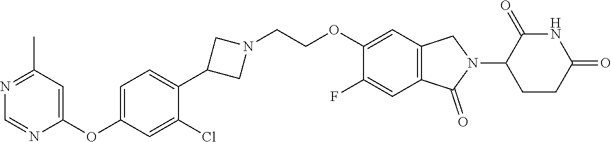 [SMILES: Cc1cc(Oc2ccc(C3CN(CCOc4cc5c(cc4F)C(=O)N(C4CCC(=O)NC4=O)C5)C3)c(Cl)c2)ncn1] | 580.2 |
| | ¹H NMR (400 MHz, DMSO-d6) δ ppm 1.98-2.01 (m, 1H), 2.38- | |
| | 2.42 (m, 1H), 2.46 (s, 4H), 2.57-2.62 (m, 1H), 2.87-2.95 (m, 1H), | |
| | 3.89-3.93 (m, 2H), 4.27-4.47 (m, 7H), 4.59 (s, 2H), 5.10 (dd, J = | |
| | 13.2, 5.2 Hz, 1H), 7.09 (s, 1H), 7.30-7.33 (m, 1H), 7.44-7.56 (m, | |
| | 3H), 7.61 (d, J = 9.6 Hz, 1H), 8.62 (s, 1H), 10.99 (s, 1H) | |
| 2-139 | 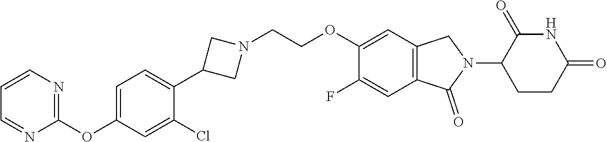 [SMILES: O=C1CCC(N2Cc3cc(OCCN4CC(c5ccc(Oc6ncccn6)cc5Cl)C4)c(F)cc3C2=O)C(=O)N1] | 566.1 |
| | ¹H NMR (400 MHz, DMSO-d6) δ ppm 1.98-2.01 (m, 1H), 2.37- | |
| | 2.42 (m, 1H), 2.57-2.61 (m, 1H), 2.85-3.03 (m, 3H), 3.35 (s, 2H), | |
| | 3.90 (s, 3H), 4.19 (s, 2H), 4.27 (d, J = 17.5 Hz, 1H), 4.39 (d, J = | |
| | 17.2 Hz, 1H), 5.06-5.11 (m, 1H), 7.23 (d, J = 8.0 Hz, 1H), 7.30 (t, | |
| | J = 4.5 Hz, 1H), 7.38 (s, 1H), 7.45 (d, J = 7.1 Hz, 1H), 7.52-7.55 | |
| | (m, 2H), 8.66 (d, J = 4.8 Hz, 2H), 10.99 (s, 1H) | |
| 2-140 | 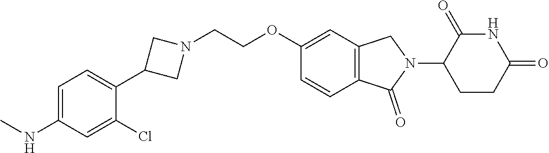 [SMILES: CNc1ccc(C2CN(CCOc3ccc4c(c3)CN(C3CCC(=O)NC3=O)C4=O)C2)c(Cl)c1] | 483.3 |
| | ¹H NMR (400 MHz, DMSO-d6) δ ppm 1.98-2.00 (m, 1H), 2.34- | |
| | 2.42 (m, 1H), 2.58-2.62 (m, 1H), 2.66 (s, 3H), 2.84-2.97 (m, 1H), | |
| | 3.68-3.81 (m, 2H), 4.12-4.54 (m, 9H), 5.09 (dd, J = 13.2, 5.2 | |
| | Hz, 1H), 6.53-6.61 (m, 2H), 7.13 (dd, J = 8.4, 2.0 Hz, 1H), 7.22- | |
| | 7.35 (m, 2H), 7.68 (d, J = 8.4 Hz, 1H), 10.99 (s, 1H) | |
| 2-141 | 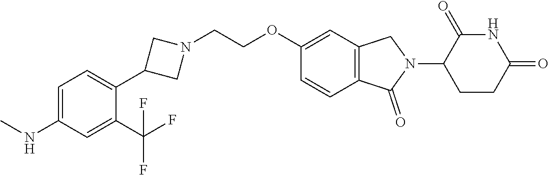 [SMILES: CNc1ccc(C2CN(CCOc3ccc4c(c3)CN(C3CCC(=O)NC3=O)C4=O)C2)c(C(F)(F)F)c1] | 517.2 |
| | ¹H NMR (400 MHz, DMSO-d6) δ ppm 1.97-2.01 (m, 1H), 2.32- | |
| | 2.41 (m, 1H), 2.57-2.62 (m, 1H), 2.69 (d, J = 4.8 Hz, 3H), 2.88- | |
| | 2.95 (m, 1H), 3.72-3.83 (m, 2H), 4.18-4.43 (m, 9H), 5.08 (dd, J = | |
| | 13.6, 8.4 Hz, 1H), 6.24 (s, 1H), 6.81-6.84 (m, 2H), 7.13 (d, J = 8.4 | |
| | Hz, 1H), 7.24 (s, 1H), 7.55 (d, J = 8.8 Hz, 1H), 7.69 (d, J = 8.4 Hz, | |
| | 1H), 10.98 (s, 1H) | |
| 2-142 | 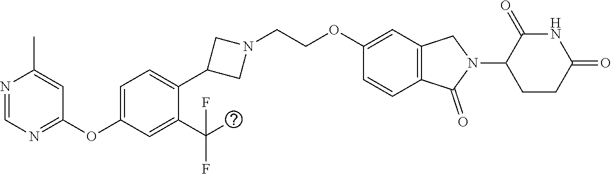 [SMILES: Cc1cc(Oc2ccc(C3CN(CCOc4ccc5c(c4)CN(C4CCC(=O)NC4=O)C5=O)C3)c(C(C)(F)F)c2)ncn1] | 596.0 |
| | ¹H NMR (400 MHz, DMSO-d6) δ ppm 1.95-2.02 (m, 1H), 2.35- | |
| | 2.42 (m, 1H), 2.46 (s, 3H), 2.57-2.61 (m, 1H), 2.87-2.95 (m, 1H), | |
| | 3.34-3.46 (m, 4H), 3.85-4.11 (m, 3H), 4.18 (s, 2H), 4.27 (d, J = | |
| | 17.3 Hz, 1H), 4.40 (d, J = 17.4 Hz, 1H), 5.07 (dd, J = 13.3, 5.1 Hz, | |
| | 1H), 7.04-7.16 (m, 2H), 7.20 (s, 1H), 7.56-7.68 (m, 3H), 7.93 | |
| | (d, J = 8.2 Hz, 1H), 8.62 (s, 1H), 10.97 (s, 1H) | |
| 2-143 | 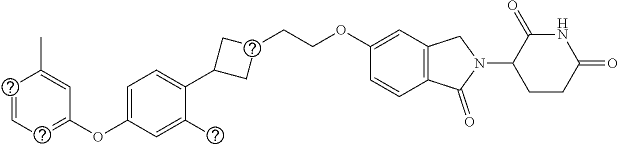 [SMILES: Cc1cccc(Oc2ccc(C3CC(CCOc4ccc5c(c4)CN(C4CCC(=O)NC4=O)C5=O)C3)c(C)c2)c1] | 562.0 |
| | ¹H NMR (400 MHz, DMSO-d6) δ ppm 1.99 (s, 1H), 2.33-2.48 | |
| | (m, 4H), 2.62 (s, 1H), 2.91 (s, 1H), 3.72 (s, 4H), 4.25-4.67 (m, | |
| | 7H), 5.09 (s, 1H), 7.08-7.32 (m, 4H), 7.44-7.91 (m, 3H), 8.63 (s, | |
| | 1H), 10.98 (s, 1H). | |
| 2-144 | 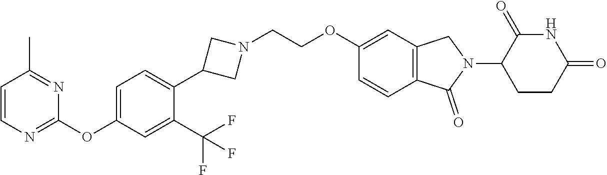 [SMILES: Cc1ccnc(Oc2ccc(C3CN(CCOc4ccc5c(c4)CN(C4CCC(=O)NC4=O)C5=O)C3)c(C(F)(F)F)c2)n1] | 596.0 |
| 2-145 | 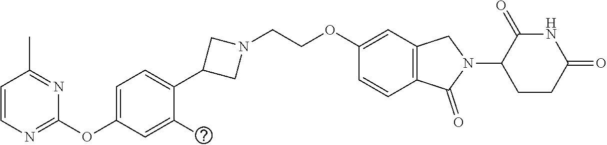 [SMILES: Cc1ccnc(Oc2ccc(C3CN(CCOc4ccc5c(c4)CN(C4CCC(=O)NC4=O)C5=O)C3)c(C)c2)n1] | 562.0 |
| | ¹H NMR (400 MHz, DMSO-d6) δ ppm 1.93-2.02 (m, 1H), 2.31- | |
| | 2.38 (m, 1H), 2.41 (s, 3H), 2.57-2.61 (m, 1H), 2.85-2.92 (m, 1H), | |
| | 2.98 (s, 1H), 3.40 (s, 2H), 3.92 (s, 3H), 4.12 (t, J = 5.2 Hz, 2H), | |
| | 4.27 (d, J = 17.2 Hz, 1H), 4.39 (d, J = 17.2 Hz, 1H), 5.07 (dd, J = | |
| | 13.4, 5.0 Hz, 1H), 7.07 (dd, J = 8.4, 2.0 Hz, 1H), 7.14-7.23 (m, | |
| | 3H), 7.34 (d, J = 2.4 Hz, 1H), 7.53 (d, J = 8.5 Hz, 1H), 7.63 (d, J = | |
| | 8.4 Hz, 1H), 8.14 (s, 1H), 8.46 (d, J = 5.0 Hz, 1H), 10.97 (s, 1H) | |
| 2-146 | 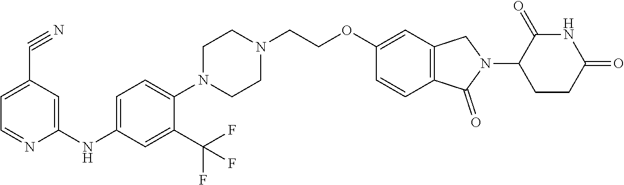 [SMILES: N#Cc1ccnc(Nc2ccc(N3CCN(CCOc4ccc5c(c4)CN(C4CCC(=O)NC4=O)C5=O)CC3)c(C(F)(F)F)c2)c1] | 634.5 |
| 2-147 | 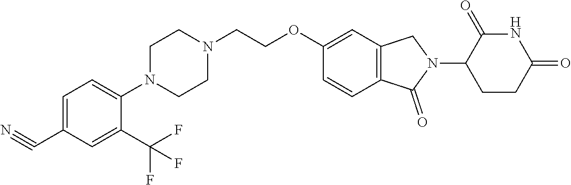 [SMILES: N#Cc1ccc(N2CCN(CCOc3ccc4c(c3)CN(C3CCC(=O)NC3=O)C4=O)CC2)c(C(F)(F)F)c1] | 542.4 |
| 2-148 | 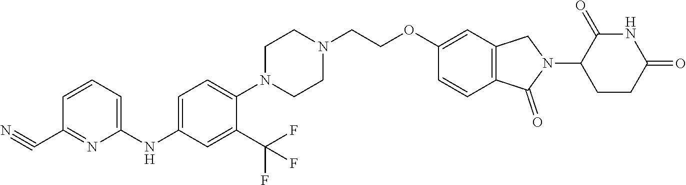 [SMILES: N#Cc1cccc(Nc2ccc(N3CCN(CCOc4ccc5c(c4)CN(C4CCC(=O)NC4=O)C5=O)CC3)c(C(F)(F)F)c2)n1] | 634.4 |
| 2-149 | 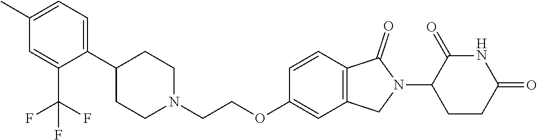 [SMILES: Cc1ccc(C2CCN(CCOc3ccc4c(c3)CN(C3CCC(=O)NC3=O)C4=O)CC2)c(C(F)(F)F)c1] | 530.4 |
| 2-150 | 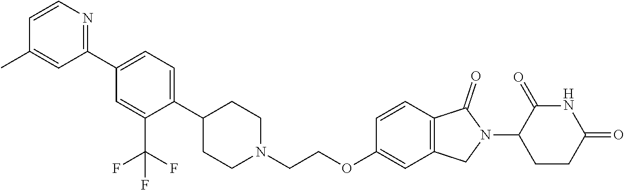 [SMILES: Cc1ccnc(-c2ccc(C3CCN(CCOc4ccc5c(c4)CN(C4CCC(=O)NC4=O)C5=O)CC3)c(C(F)(F)F)c2)c1] | 606.4 |
| 2-151 | 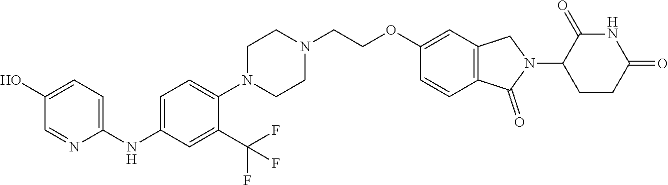 [SMILES: O=C1CCC(N2Cc3cc(OCCN4CCN(c5ccc(Nc6ccc(O)cn6)cc5C(F)(F)F)CC4)ccc3C2=O)C(=O)N1] | 625.5 |
| 2-152 | 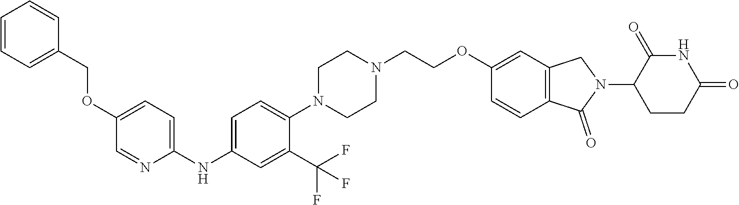 [SMILES: O=C1CCC(N2Cc3cc(OCCN4CCN(c5ccc(Nc6ccc(OCc7ccccc7)cn6)cc5C(F)(F)F)CC4)ccc3C2=O)C(=O)N1] | 715.5 |
| 2-153 | 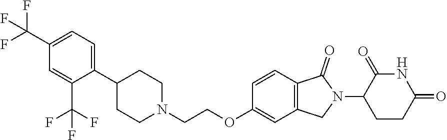 [SMILES: O=C1CCC(N2Cc3cc(OCCN4CCC(c5ccc(C(F)(F)F)cc5C(F)(F)F)CC4)ccc3C2=O)C(=O)N1] | 584.4 |
| 2-154 | 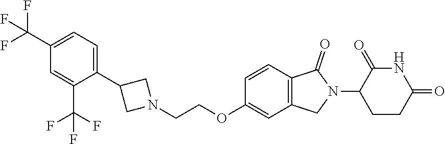 [SMILES: O=C1CCC(N2Cc3cc(OCCN4CC(c5ccc(C(F)(F)F)cc5C(F)(F)F)C4)ccc3C2=O)C(=O)N1] | 556.4 |
| 2-155 | 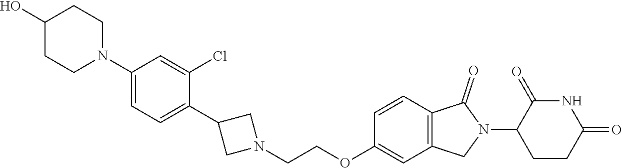 [SMILES: O=C1CCC(N2Cc3cc(OCCN4CC(c5ccc(N6CCC(O)CC6)cc5Cl)C4)ccc3C2=O)C(=O)N1] | 553.3, 277.3 |
| 2-156 | 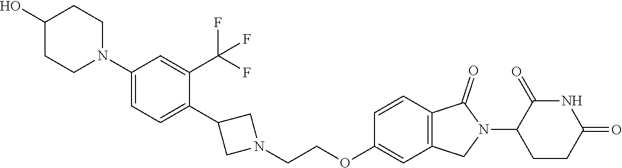 [SMILES: O=C1CCC(N2Cc3cc(OCCN4CC(c5ccc(N6CCC(O)CC6)cc5C(F)(F)F)C4)ccc3C2=O)C(=O)N1] | 294.2, 587.3 |
| 2-157 | 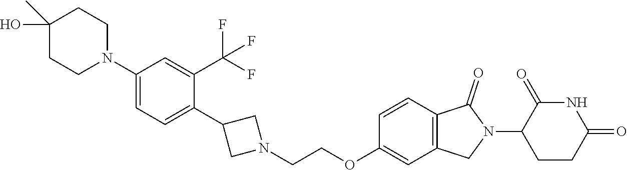 [SMILES: CC1(O)CCN(c2ccc(C3CN(CCOc4ccc5c(c4)CN(C4CCC(=O)NC4=O)C5=O)C3)c(C(F)(F)F)c2)CC1] | 301.3, 601.5 |
| 2-158 | 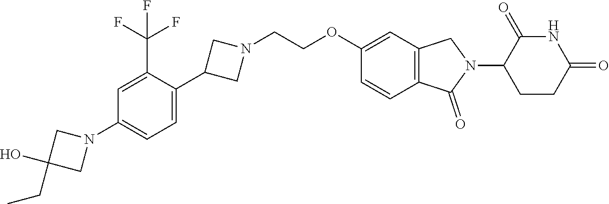 [SMILES: CCC1(O)CN(c2ccc(C3CN(CCOc4ccc5c(c4)CN(C4CCC(=O)NC4=O)C5=O)C3)c(C(F)(F)F)c2)C1] | 587.4, 294.2 |
| 2-159 | 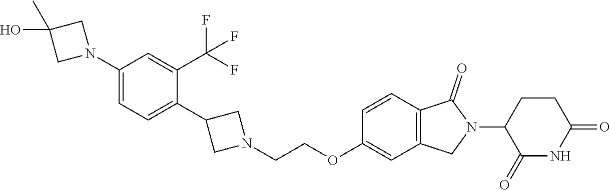 [SMILES: CC1(O)CN(c2ccc(C3CN(CCOc4ccc5c(c4)CN(C4CCC(=O)NC4=O)C5=O)C3)c(C(F)(F)F)c2)C1] | 287.2, 573.3 |
| 2-160 | 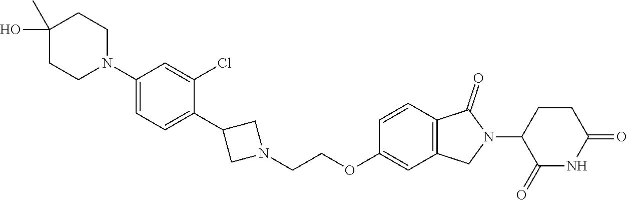 [SMILES: CC1(O)CCN(c2ccc(C3CN(CCOc4ccc5c(c4)CN(C4CCC(=O)NC4=O)C5=O)C3)c(Cl)c2)CC1] | 567.3, 284.2 |
| 2-161 | 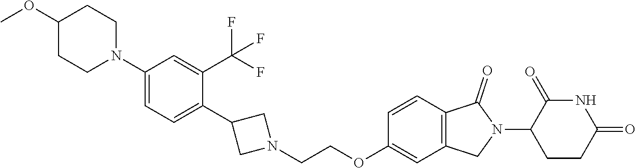 [SMILES: COC1CCN(c2ccc(C3CN(CCOc4ccc5c(c4)CN(C4CCC(=O)NC4=O)C5=O)C3)c(C(F)(F)F)c2)CC1] | 301.3, 601.3 |
| 2-162 | 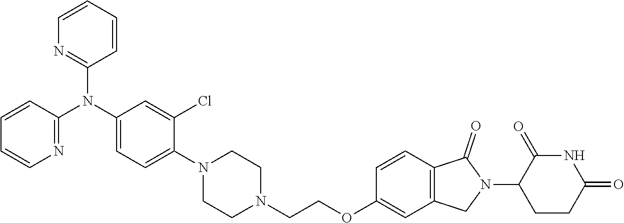 [SMILES: O=C1CCC(N2Cc3cc(OCCN4CCN(c5ccc(N(c6ccccn6)c6ccccn6)cc5Cl)CC4)ccc3C2=O)C(=O)N1] | 652.3, 326.8 |
| 2-163 | 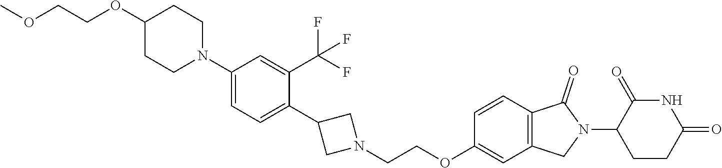 [SMILES: COCCOC1CCN(c2ccc(C3CN(CCOc4ccc5c(c4)CN(C4CCC(=O)NC4=O)C5=O)C3)c(C(F)(F)F)c2)CC1] | 645.4, 323.3 |
| 2-164 | 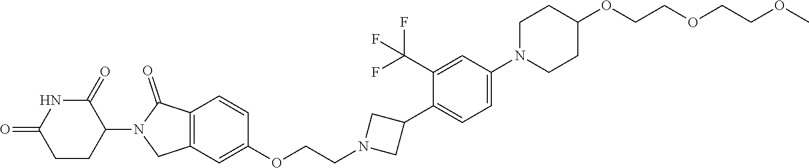 [SMILES: COCCOCCOC1CCN(c2ccc(C3CN(CCOc4ccc5c(c4)CN(C4CCC(=O)NC4=O)C5=O)C3)c(C(F)(F)F)c2)CC1] | 689.5, 345.2 |
| 2-165 | 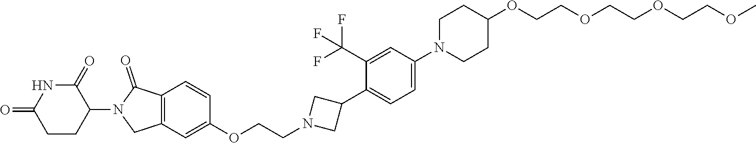 [SMILES: COCCOCCOCCOC1CCN(c2ccc(C3CN(CCOc4ccc5c(c4)CN(C4CCC(=O)NC4=O)C5=O)C3)c(C(F)(F)F)c2)CC1] | 733.6, 367.3 |
| 2-166 | 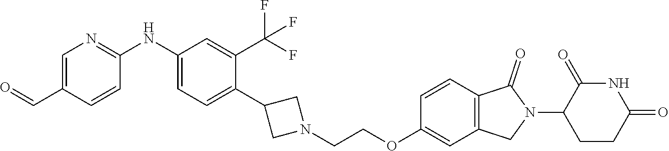 [SMILES: O=Cc1ccc(Nc2ccc(C3CN(CCOc4ccc5c(c4)CN(C4CCC(=O)NC4=O)C5=O)C3)c(C(F)(F)F)c2)nc1] | 608.4 |
| 2-168 | 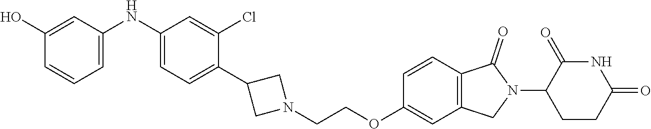 [SMILES: O=C1CCC(N2Cc3cc(OCCN4CC(c5ccc(Nc6cccc(O)c6)cc5Cl)C4)ccc3C2=O)C(=O)N1] | 561.4 |
| 2-169 | 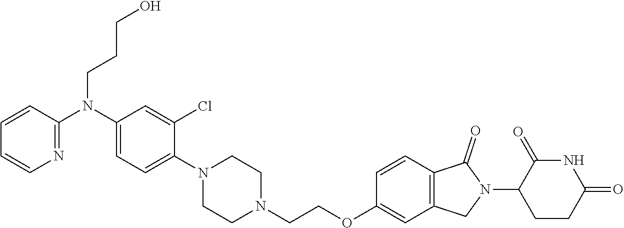 [SMILES: O=C1CCC(N2Cc3cc(OCCN4CCN(c5ccc(N(CCCO)c6ccccn6)cc5Cl)CC4)ccc3C2=O)C(=O)N1] | 317.1, 633.3 |
| | ¹H NMR (400 MHz, DMSO_d6) δ 1.62-1.73 (m, 2H), 1.93- | |
| | 2.04 (m, 1H), 2.36-2.40 (m, 1H), 2.55-2.63 (m, 1H), 2.69 (s, | |
| | 4H), 2.82 (t, J = 5.6 Hz, 2H), 2.87-2.93 (m, 1H), 3.02 (s, 4H), | |
| | 3.43 (t, J = 6.0 Hz, 2H), 3.85-3.94 (m, 2H), 4.18-4.32 (m, 3H), | |
| | 4.39 (d, J = 17.2 Hz, 1H), 4.56 (s, 1H), 5.08 (dd, J = 13.2, 5.2 Hz, | |
| | 1H), 6.37 (d, J = 8.4 Hz, 1H), 6.61-6.66 (m, 1H), 7.08 (dd, J = | |
| | 8.4, 2.0 Hz, 1H), 7.14-7.26 (m, 3H), 7.32 (d, J = 1.2 Hz, 1H), 7.38- | |
| | 7.43 (m, 1H), 7.63 (d, J = 8.4 Hz, 1H), 8.11 (dd, J = 5.2, 1.2 Hz, | |
| | 1H), 10.97 (s, 1H). | |
| 2-170 | 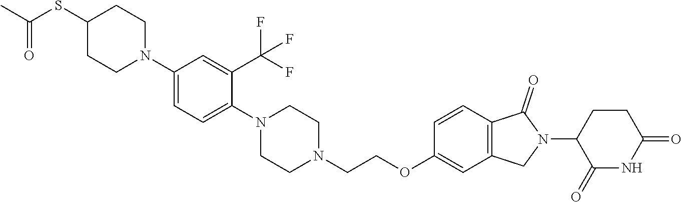 [SMILES: CC(=O)SC1CCN(c2ccc(N3CCN(CCOc4ccc5c(c4)CN(C4CCC(=O)NC4=O)C5=O)CC3)c(C(F)(F)F)c2)CC1] | 632.3, 316.7 |
| | ¹H NMR (400 MHz, DMSO_d6) δ 1.57-1.69 (m, 2H), 1.92-2.02 | |
| | (m, 3H), 2.32 (s, 3H), 2.36-2.43 (m, 1H), 2.56-2.66 (m, 1H), | |
| | 2.86-2.94 (m, 2H), 2.93-3.05 (m, 5H), 3.08-3.15 (m, 2H), 3.53- | |
| | 3.59 (m, 4H), 3.70 (s, 3H), 4.29 (d, J = 17.2 Hz, 1H), 4.40-4.54 | |
| | (m, 3H), 5.08 (dd, J = 13.2, 5.2 Hz, 1H), 7.11-7.17 (m, 2H), 7.21- | |
| | 7.27 (m, 2H), 7.41 (d, J = 9.2 Hz, 1H), 7.69 (d, J = 8.4 Hz, 1H), | |
| | 9.94 (br. s, 1H), 10.98 (s, 1H). | |
| 2-171 | 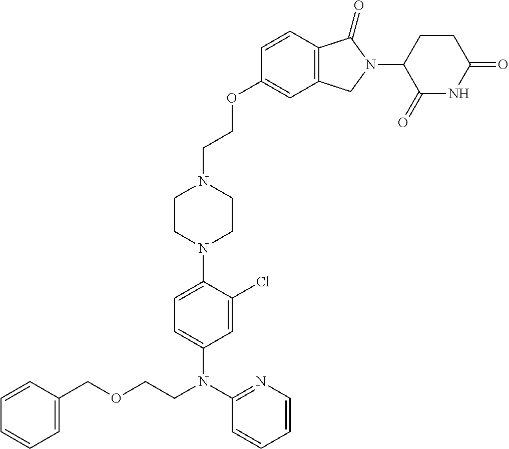 [SMILES: O=C1CCC(N2Cc3cc(OCCN4CCN(c5ccc(N(CCOCc6ccccc6)c6ccccn6)cc5Cl)CC4)ccc3C2=O)C(=O)N1] | 709.6, 711.6 |
| 2-172 | 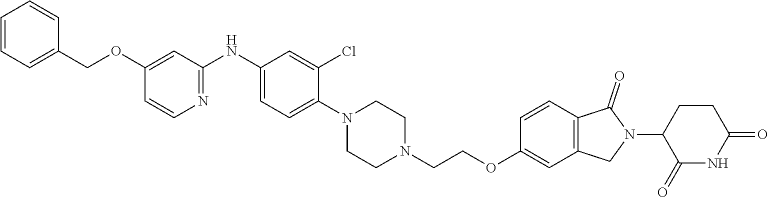 [SMILES: O=C1CCC(N2Cc3cc(OCCN4CCN(c5ccc(Nc6cc(OCc7ccccc7)ccn6)cc5Cl)CC4)ccc3C2=O)C(=O)N1] | 681.4 |
| 2-173 | 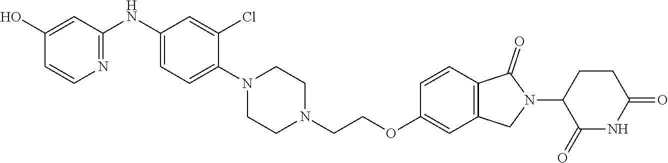 [SMILES: O=C1CCC(N2Cc3cc(OCCN4CCN(c5ccc(Nc6cc(O)ccn6)cc5Cl)CC4)ccc3C2=O)C(=O)N1] | 591.0, 592.5 |
| 2-174 | 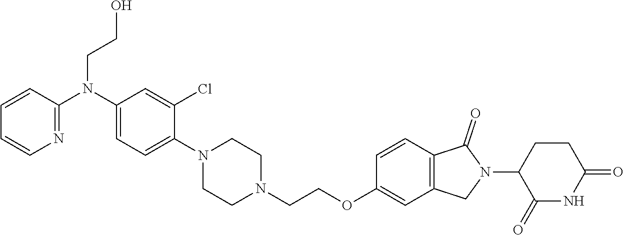 [SMILES: O=C1CCC(N2Cc3cc(OCCN4CCN(c5ccc(N(CCO)c6ccccn6)cc5Cl)CC4)ccc3C2=O)C(=O)N1] | 619.0, 621.0 |
| 2-175 | 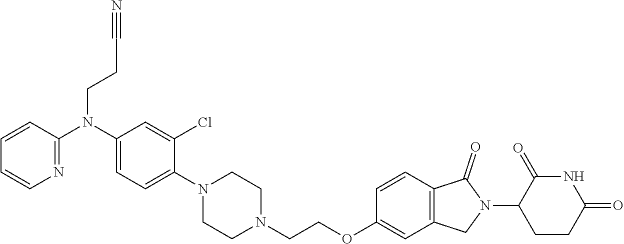 [SMILES: N#CCCN(c1ccc(N2CCN(CCOc3ccc4c(c3)CN(C3CCC(=O)NC3=O)C4=O)CC2)c(Cl)c1)c1ccccn1] | 628.5, 630.4 |
| 2-176 | 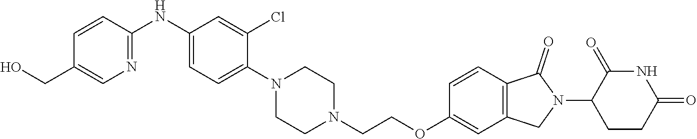 [SMILES: O=C1CCC(N2Cc3cc(OCCN4CCN(c5ccc(Nc6ccc(CO)cn6)cc5Cl)CC4)ccc3C2=O)C(=O)N1] | 605.3, 607.2 |
| 2-177 | 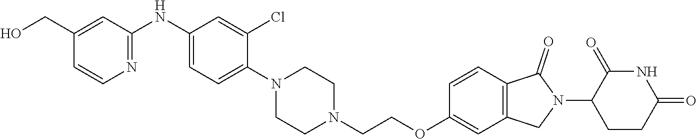 [SMILES: O=C1CCC(N2Cc3cc(OCCN4CCN(c5ccc(Nc6cc(CO)ccn6)cc5Cl)CC4)ccc3C2=O)C(=O)N1] | 605.4, 607.4 |
| 2-178 | 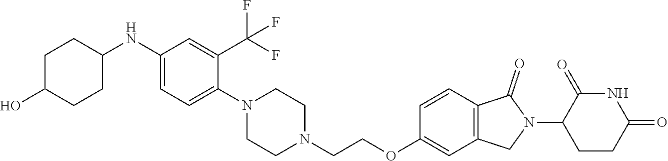 [SMILES: O=C1CCC(N2Cc3cc(OCCN4CCN(c5ccc(NC6CCC(O)CC6)cc5C(F)(F)F)CC4)ccc3C2=O)C(=O)N1] | 630.4 |
| 2-179 | 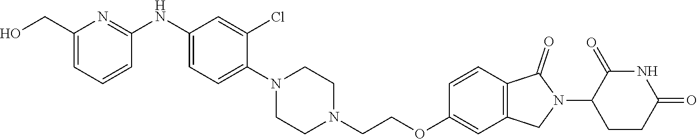 [SMILES: O=C1CCC(N2Cc3cc(OCCN4CCN(c5ccc(Nc6cccc(CO)n6)cc5Cl)CC4)ccc3C2=O)C(=O)N1] | 605.3, 607.3 |
| 2-180 | 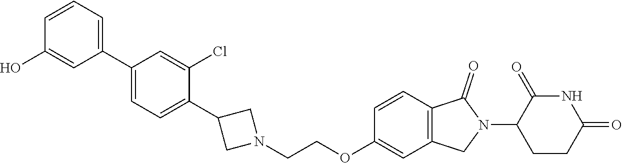 [SMILES: O=C1CCC(N2Cc3cc(OCCN4CC(c5ccc(-c6cccc(O)c6)cc5Cl)C4)ccc3C2=O)C(=O)N1] | 546.3, 548.2 |
| 2-181 | 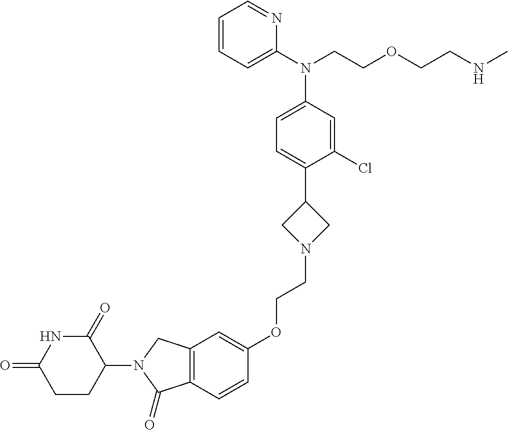 [SMILES: CNCCOCCN(c1ccc(C2CN(CCOc3ccc4c(c3)CN(C3CCC(=O)NC3=O)C4=O)C2)c(Cl)c1)c1ccccn1] | 647.4, 324.3 |
| 2-182 | 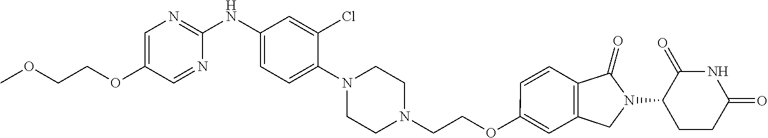 [SMILES: COCCOc1cnc(Nc2ccc(N3CCN(CCOc4ccc5c(c4)CN([C@H]4CCC(=O)NC4=O)C5=O)CC3)c(Cl)c2)nc1] | 650.6 |
| 2-183 | 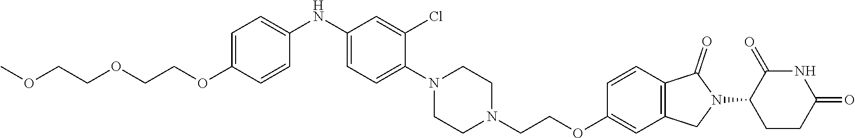 [SMILES: COCCOCCOc1ccc(Nc2ccc(N3CCN(CCOc4ccc5c(c4)CN([C@H]4CCC(=O)NC4=O)C5=O)CC3)c(Cl)c2)cc1] | 692.3 |
| 2-184 | 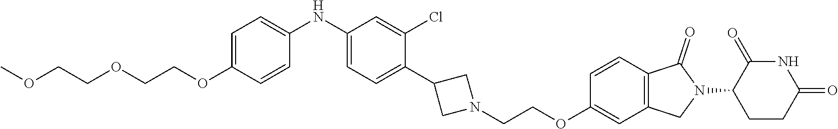 [SMILES: COCCOCCOc1ccc(Nc2ccc(C3CN(CCOc4ccc5c(c4)CN([C@H]4CCC(=O)NC4=O)C5=O)C3)c(Cl)c2)cc1] | 663.3 |
| 2-185 | 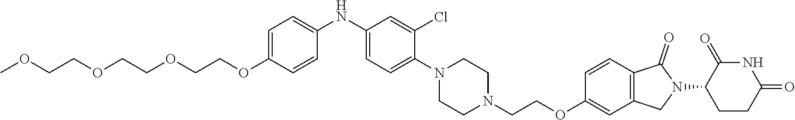 [SMILES: COCCOCCOCCOc1ccc(Nc2ccc(N3CCN(CCOc4ccc5c(c4)CN([C@H]4CCC(=O)NC4=O)C5=O)CC3)c(Cl)c2)cc1] | 736.4 |
| 2-186 | 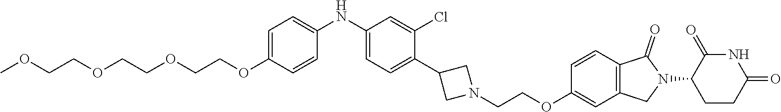 [SMILES: COCCOCCOCCOc1ccc(Nc2ccc(C3CN(CCOc4ccc5c(c4)CN([C@H]4CCC(=O)NC4=O)C5=O)C3)c(Cl)c2)cc1] | 707.4 |
| 2-187 | 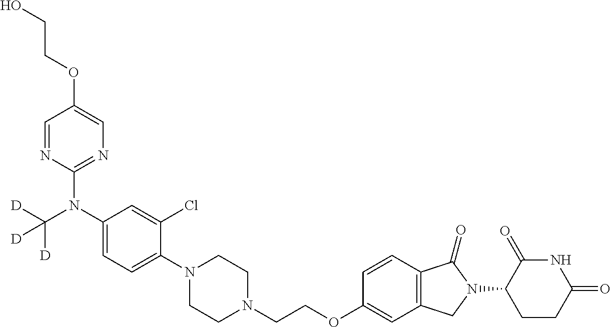 [SMILES: [2H]C([2H])([2H])N(c1ccc(N2CCN(CCOc3ccc4c(c3)CN([C@H]3CCC(=O)NC3=O)C4=O)CC2)c(Cl)c1)c1ncc(OCCO)cn1] | 653.4 |

Alternatively, racemic mixtures of compounds were separated by supercritical fluid chromatography as below.

### Compounds 2-4 and 2-45

A sample of racemic Compound 2-4 was separated by supercritical fluid chromatography under the following conditions: Column, CHIRALPAK OJ 5 μm 30*250 mm; Mobile phase, 40% Ethanol in CO₂; Flow rate, 70 mL/min; Sample, 50.0 mg dissolved in 5.5 mL ACN; Injection, 5 mL; Detection: 220 nm.

Compound 2-17: first-eluting peak, 8.4 mg, 100% purity, 100% ee. LC/MS found 452.2 [M+H]⁺.

Compound 2-18: second-eluting peak, 8.7 mg, 100% purity, 96.4% ee. LC/MS found 452.2 [M+H]⁺.

A sample of racemic Compound 2-45 was separated by supercritical fluid chromatography under the following conditions: Column, ChiralPak IC-H 21×250 mm; Mobile phase, 35% methanol in CO₂; Flow rate, 70 mL/min; Sample, 25.5 mg was dissolved in 2.0 mL methanol and 2.0 mL dichloromethane; Injection, 1.0 mL; Detection, 254 nm.

Compound 2-21: first-eluting peak, 10.9 mg, 100% purity, 100% ee. LC/MS found 436.2 [M+H]⁺.

Compound 2-22: second-eluting peak, 8.7 mg, 100% purity, 96.4% ee. LC/MS found 452.2 [M+H]⁺.

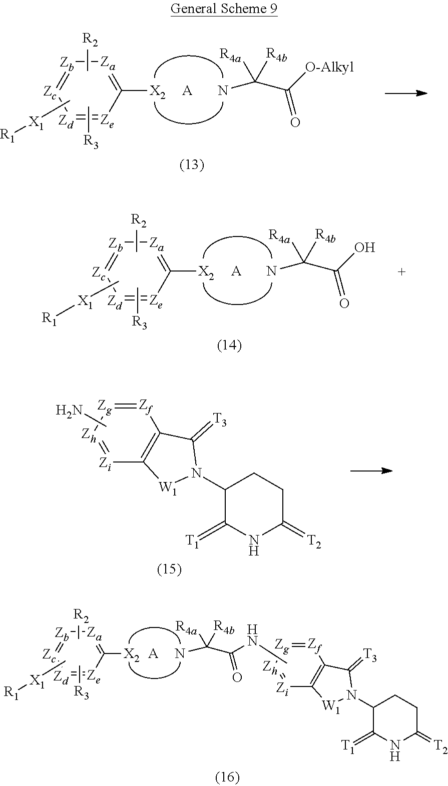

### Method 47

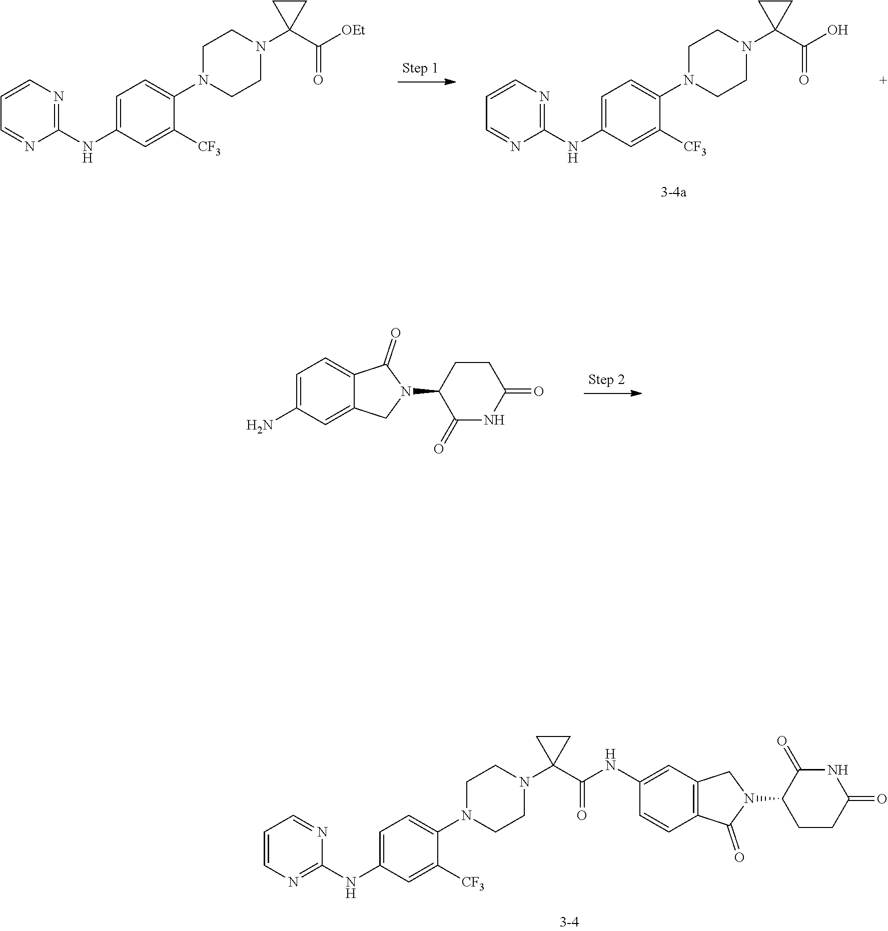 [SMILES: CCOC(=O)C1(N2CCN(c3ccc(Nc4ncccn4)cc3C(F)(F)F)CC2)CC1 ; O=C(O)C1(N2CCN(c3ccc(Nc4ncccn4)cc3C(F)(F)F)CC2)CC1 ; Nc1ccc2c(c1)CN([C@H]1CCC(=O)NC1=O)C2=O ; O=C1CC[C@H](N2Cc3cc(NC(=O)C4(N5CCN(c6ccc(Nc7ncccn7)cc6C(F)(F)F)CC5)CC4)ccc3C2=O)C(=O)N1]

Step 1: To a mixture of ethyl 1-(4-(4-(pyrimidin-2-ylamino)-2-(trifluoromethyl)-phenyl)piperazin-1-yl)cyclopropane-1-carboxylate (180 mg, 413.37 μmol) in EtOH (10 mL) was added KOH (231.93 mg, 4.13 mmol). The resulting mixture was stirred at 90° C. for 18 hours. AcOH (248.24 mg, 4.13 mmol) was added, and the solid was filtered off and solvent was removed. The crude product was purified by silica gel column (DCM/MeOH=10:1) to give 3-4a (157 mg, 93% yield). ESI-MS: m/z=408.3 [M+1]⁺.
Step 2: A solution of 3-4a (30 mg, 73.64 μmol), (3S)-3-(5-amino-1-oxo-isoindolin-2-yl)piperidine-2,6-dione (21 mg, 81.00 μmol), HATU (56 mg, 147.28 μmol), and DIPEA (47.59 mg, 368.20 μmol) in DMF (5 mL) was stirred at room temperature for 18 hours. The reaction mixture was then concentrated in vacuo and purified by silica gel chromatography (DCM/MeOH=30:1) to obtain 3-4 (13.8 mg, 28% yield). ESI-MS: m/z=649.5 [M+1]⁺.

The following compounds (16) 3-1 to 3-6 shown in Table 3 were prepared by methods similar to those described for the preparation of compound 3-3, using the appropriate carboxylic acid intermediate (14).

TABLE 3

| | | Product | |
|---|---|---|---|
| Compd | 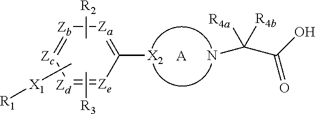 | 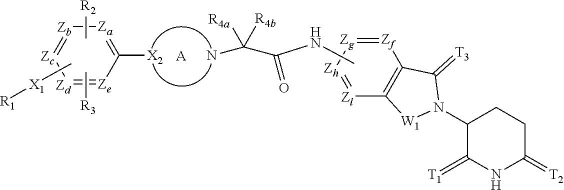 | MS found [M + H]+ |
| 3-1 | 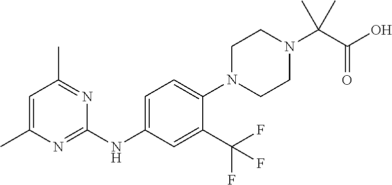 [SMILES: Cc1cc(C)nc(Nc2ccc(N3CCN(C(C)(C)C(=O)O)CC3)c(C(F)(F)F)c2)n1] | 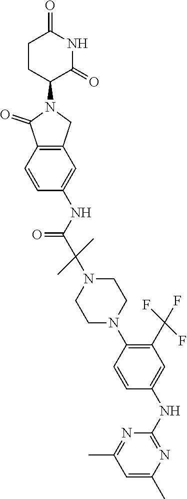 [SMILES: Cc1cc(C)nc(Nc2ccc(N3CCN(C(C)(C)C(=O)Nc4ccc5c(c4)CN([C@H]4CCC(=O)NC4=O)C5=O)CC3)c(C(F)(F)F)c2)n1] | 679.5 |
| 3-2 | 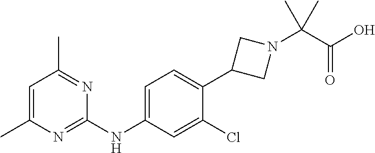 [SMILES: Cc1cc(C)nc(Nc2ccc(C3CN(C(C)(C)C(=O)O)C3)c(Cl)c2)n1] | 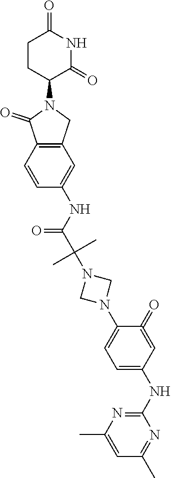 | 616.4 |
| 3-3 | 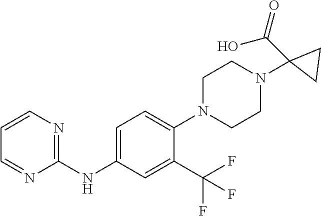 [SMILES: O=C(O)C1(N2CCN(c3ccc(Nc4ncccn4)cc3C(F)(F)F)CC2)CC1] | 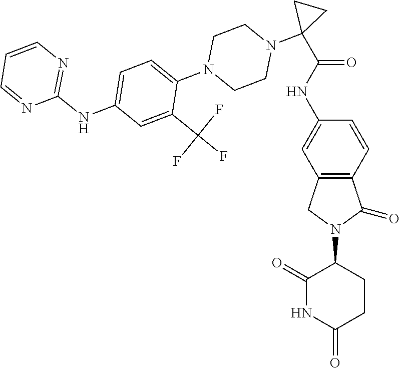 [SMILES: O=C1CC[C@H](N2Cc3cc(NC(=O)C4(N5CCN(c6ccc(Nc7ncccn7)cc6C(F)(F)F)CC5)CC4)ccc3C2=O)C(=O)N1] | 649.5 |
| 3-4 | 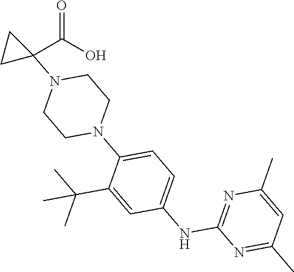 [SMILES: Cc1cc(C)nc(Nc2ccc(N3CCN(C4(C(=O)O)CC4)CC3)c(C(C)(C)C)c2)n1] | 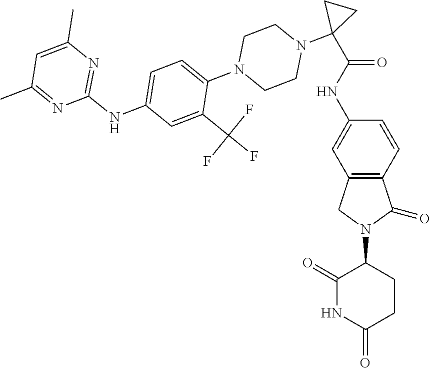 [SMILES: Cc1cc(C)nc(Nc2ccc(N3CCN(C4(C(=O)Nc5ccc6c(c5)CN([C@H]5CCC(=O)NC5=O)C6=O)CC4)CC3)c(C(F)(F)F)c2)n1] | 677.6 |
| 3-5 | 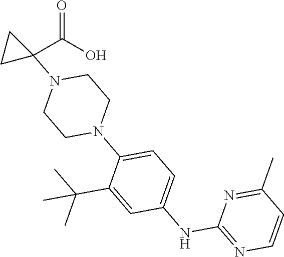 [SMILES: Cc1ccnc(Nc2ccc(N3CCN(C4(C(=O)O)CC4)CC3)c(C(C)(C)C)c2)n1] | 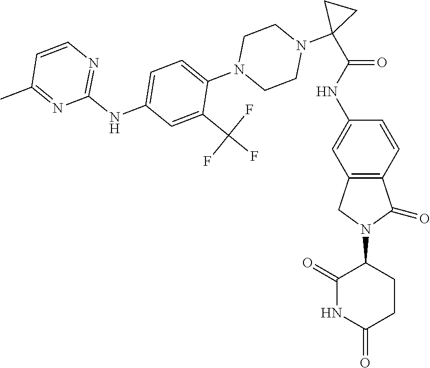 [SMILES: Cc1ccnc(Nc2ccc(N3CCN(C4(C(=O)Nc5ccc6c(c5)CN([C@H]5CCC(=O)NC5=O)C6=O)CC4)CC3)c(C(F)(F)F)c2)n1] | 663.5 |
| 3-6 | 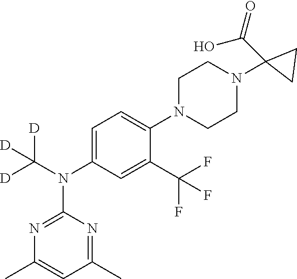 [SMILES: [2H]C([2H])([2H])N(c1ccc(N2CCN(C3(C(=O)O)CC3)CC2)c(C(F)(F)F)c1)c1nc(C)cc(C)n1] | 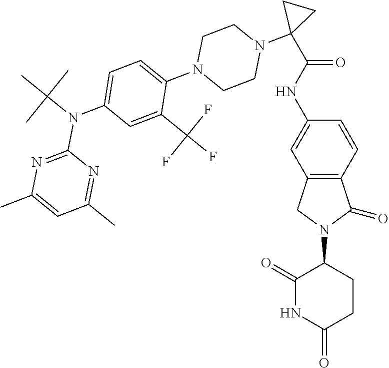 [SMILES: Cc1cc(C)nc(N(c2ccc(N3CCN(C4(C(=O)Nc5ccc6c(c5)CN([C@H]5CCC(=O)NC5=O)C6=O)CC4)CC3)c(C(F)(F)F)c2)C(C)(C)C)n1] | 694.6 |

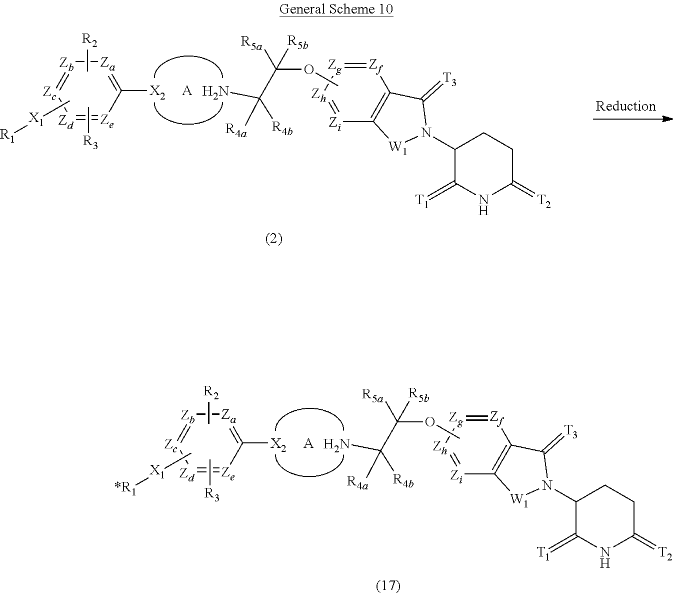

### Method 48

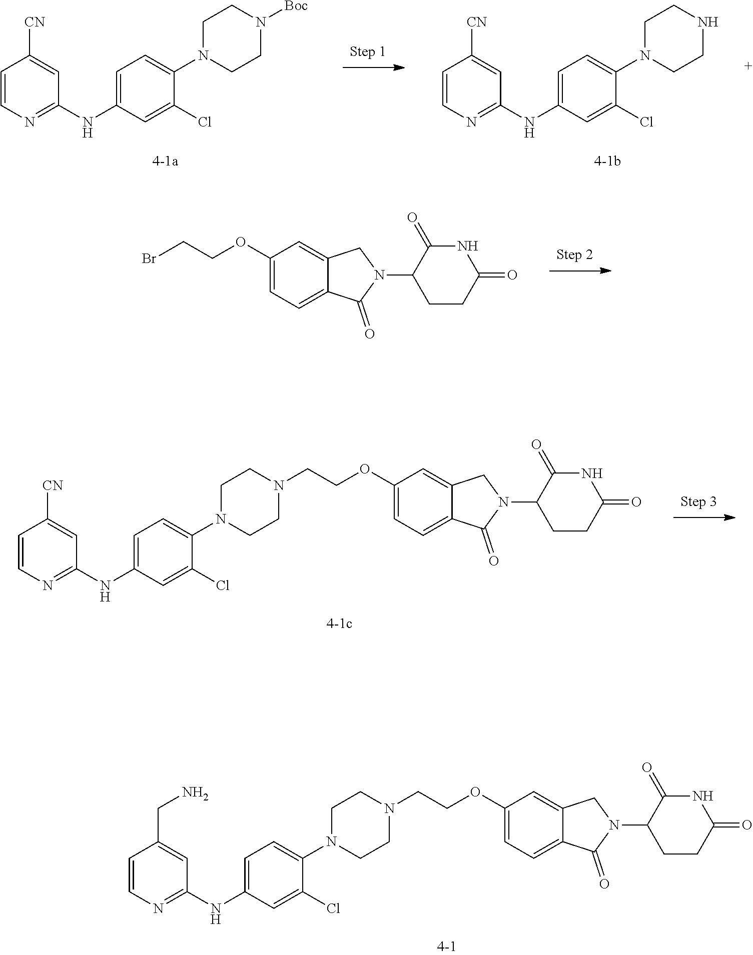 [SMILES: CC(C)(C)OC(=O)N1CCN(c2ccc(Nc3cc(C#N)ccn3)cc2Cl)CC1 ; N#Cc1ccnc(Nc2ccc(N3CCNCC3)c(Cl)c2)c1 ; O=C1CCC(N2Cc3cc(OCCBr)ccc3C2=O)C(=O)N1 ; N#Cc1ccnc(Nc2ccc(N3CCN(CCOc4ccc5c(c4)CN(C4CCC(=O)NC4=O)C5=O)CC3)c(Cl)c2)c1 ; NCc1ccnc(Nc2ccc(N3CCN(CCOc4ccc5c(c4)CN(C4CCC(=O)NC4=O)C5=O)CC3)c(Cl)c2)c1]

Step 1: To a solution of tert-butyl 4-[4-[(4-cyano-2-pyridyl)amino]-2-(trifluoromethyl)phenyl]-piperazine-1-carboxylate (910 mg, 2.03 mmol) in DCM (5 mL) was added TFA (2.32 g, 20.34 mmol) dropwise at 0° C. The reaction mixture was stirred at room temperature for 2 hours. The reaction mixture was then concentrated in vacuo and purified by silica gel chromatography (DCM/MeOH=10:1) to give 4-1b (760 mg, 81% yield). ESI-MS: m/z=348.2 [M+1]⁺.
Step 2: A solution of 4-1b (200 mg, 575.81 μmol), 3-[5-(2-bromoethoxy)-1-oxo-isoindolin-2-yl]piperidine-2,6-dione (232.58 mg, 633.39 μmol) and TEA (233.06 mg, 2.30 mmol) in DMF (2 mL) was stirred at 50° C. for 16 hours. The reaction mixture was then concentrated in vacuo and purified by silica gel chromatography (DCM/MeOH=30:1 to 15:1) to give 4-1c (306 mg, 83% yield). ESI-MS: m/z=634.5 [M+1]⁺.
Step 3: A solution of 4-1c (150 mg, 236.74 μmol), 10% Pd/C (12.60 mg, 118.37 μmol) in EtOAc/MeOH (1:1, 10 mL) was stirred at room temperature overnight under hydrogen atmosphere.

The reaction mixture was Celite filter and concentrated in vacuo and purified by silica gel chromatography (DCM/MeOH=4:6) to give 4-1 (26.4 mg, 17% yield). ESI-MS: m/z=638.6 [M+1]⁺.

The following compounds 4-1 through 4-2 (17) shown in Table 4 were prepared by methods similar to those described for the preparation of 4-1, using the appropriate nitrile intermediate (9).

TABLE 4

| | | Product | |
|---|---|---|---|
| Compd | 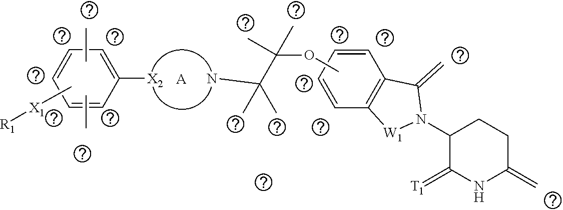 | 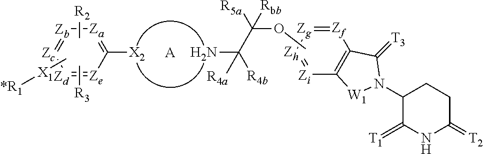 | MS found [M + H]+ |
| 4-1 | 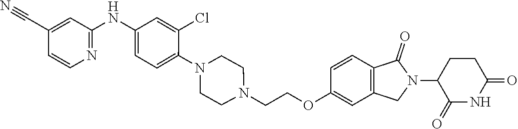 [SMILES: N#Cc1ccnc(Nc2ccc(N3CCN(CCOc4ccc5c(c4)CN(C4CCC(=O)NC4=O)C5=O)CC3)c(Cl)c2)c1] | 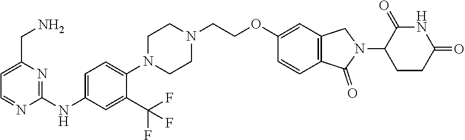 [SMILES: NCc1ccnc(Nc2ccc(N3CCN(CCOc4ccc5c(c4)CN(C4CCC(=O)NC4=O)C5=O)CC3)c(C(F)(F)F)c2)n1] | 511.2 513.2 |
| 4-2 | 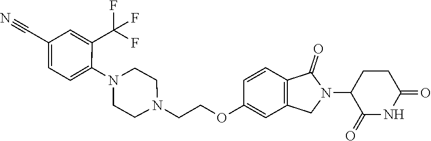 [SMILES: N#Cc1ccc(N2CCN(CCOc3ccc4c(c3)CN(C3CCC(=O)NC3=O)C4=O)CC2)c(C(F)(F)F)c1] | 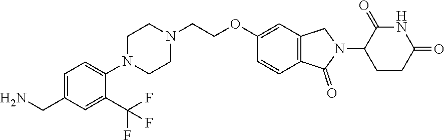 [SMILES: NCc1ccc(N2CCN(CCOc3ccc4c(c3)CN(C3CCC(=O)NC3=O)C4=O)CC2)c(C(F)(F)F)c1] | 480.3 |

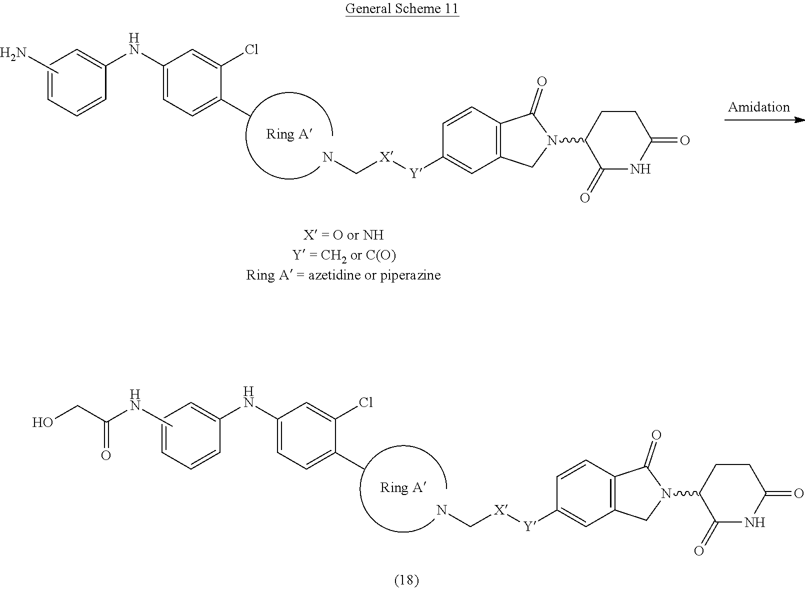

### Method 49

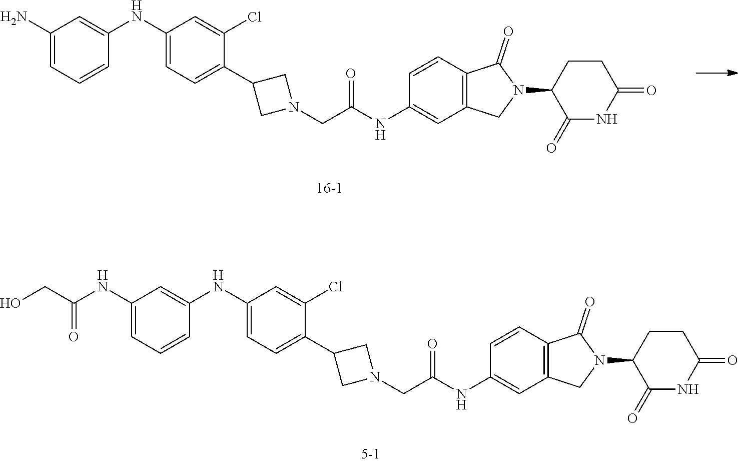 [SMILES: Nc1cccc(Nc2ccc(C3CN(CC(=O)Nc4ccc5c(c4)CN([C@H]4CCC(=O)NC4=O)C5=O)C3)c(Cl)c2)c1 ; O=C1CC[C@H](N2Cc3cc(NC(=O)CN4CC(c5ccc(Nc6cccc(NC(=O)CO)c6)cc5Cl)C4)ccc3C2=O)C(=O)N1]

A solution of [3-[3-chloro-4-[1-[2-[[2-[(3S)-2,6-dioxo-3-piperidyl]-1-oxo-isoindolin-5-yl]amino]-2-oxo-ethyl]azetidin-3-yl]anilino]phenyl]azinate (62.99 mg, 104.28 μmol) in EtOH (2 mL) and THF (1 mL) was treated with dichlorotin;dihydrate (117.65 mg, 521.39 μmol), then the mixture was stirred for overnight at room temperature. The mixture was filtered via a cellite, then the filtrate was collected, concentrated, and purified by a flash column to afford 15 {2-[3-[4-(3-aminoanilino)-2-chloro-phenyl]azetidin-1-yl]-N-[2-[(3S)-2,6-dioxo-3-piperidyl]-1-oxo-isoindolin-5-yl]acetamide (5-1, 8 mg, 9.07 μmol, 8.70% yield, 65% purity)}. MeOH (0 to 30%) in EtOAc. ESI-MS: m/z=573.4[M+1]⁺

The following compounds 5-1 through 5-2 (18) shown in Table 5 were prepared by methods similar to those described for the preparation of 5-1, using the appropriate the amine intermediate.

TABLE 5

| | Starting material | Product | |
|---|---|---|---|
| Compd | 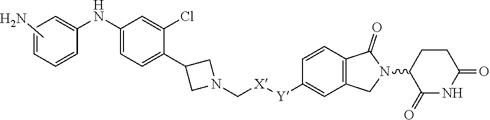 | 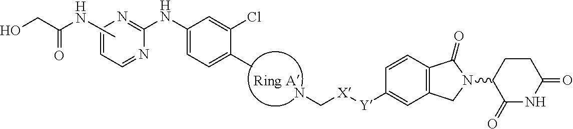 | MS found [M + H]+ |
| 5-1 | 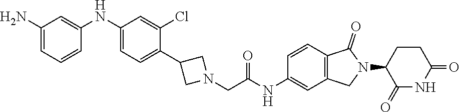 [SMILES: Nc1cccc(Nc2ccc(C3CN(CC(=O)Nc4ccc5c(c4)CN([C@H]4CCC(=O)NC4=O)C5=O)C3)c(Cl)c2)c1] | 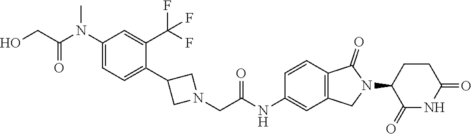 [SMILES: CN(C(=O)CO)c1ccc(C2CN(CC(=O)Nc3ccc4c(c3)CN([C@H]3CCC(=O)NC3=O)C4=O)C2)c(C(F)(F)F)c1] | 588.4 |
| 5-2 | 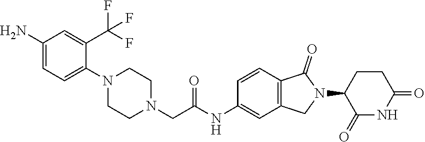 [SMILES: Nc1ccc(N2CCN(CC(=O)Nc3ccc4c(c3)CN([C@H]3CCC(=O)NC3=O)C4=O)CC2)c(C(F)(F)F)c1] | 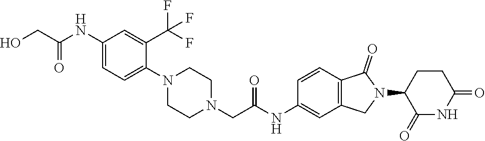 [SMILES: O=C1CC[C@H](N2Cc3cc(NC(=O)CN4CCN(c5ccc(NC(=O)CO)cc5C(F)(F)F)CC4)ccc3C2=O)C(=O)N1] | 603.4 |

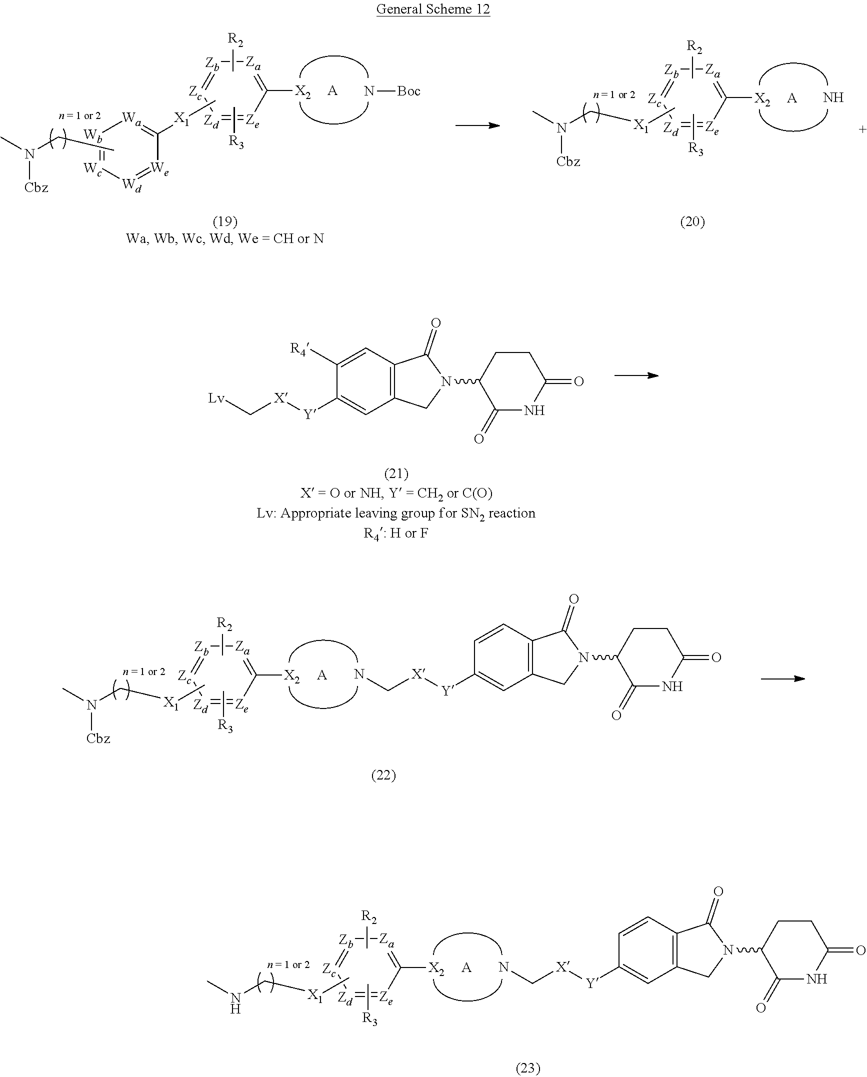

### Method 50

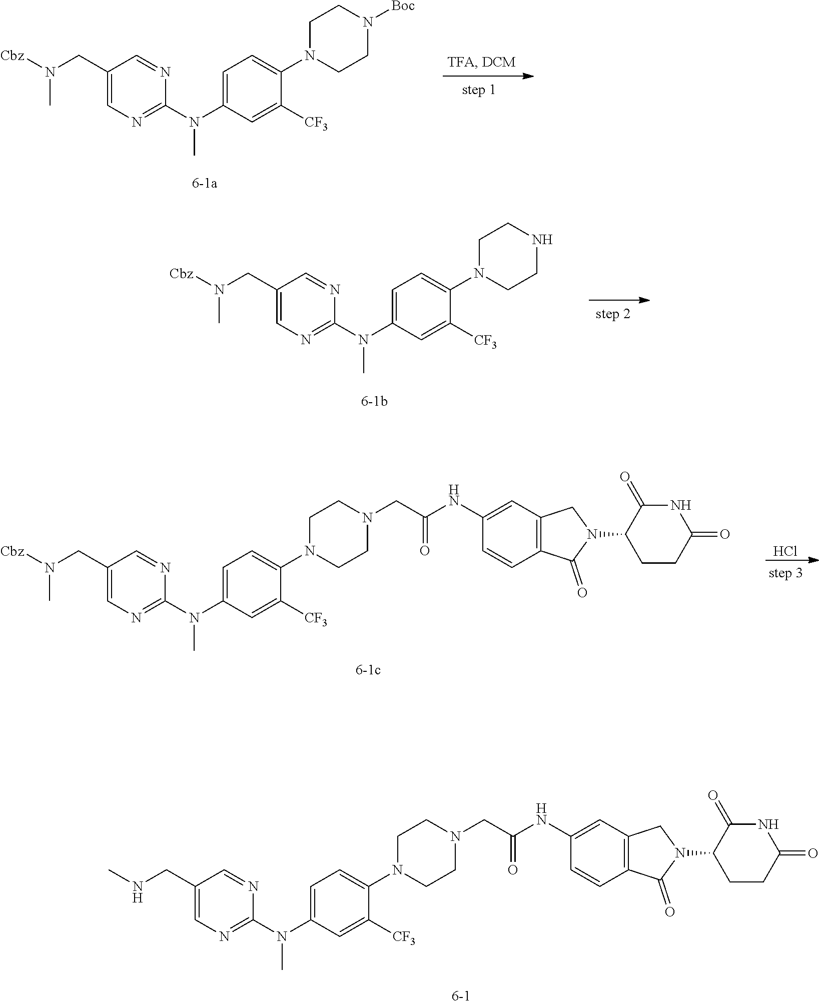 [SMILES: CN(Cc1cnc(N(C)c2ccc(N3CCN(C(=O)OC(C)(C)C)CC3)c(C(F)(F)F)c2)nc1)C(=O)OCc1ccccc1 ; CN(Cc1cnc(N(C)c2ccc(N3CCNCC3)c(C(F)(F)F)c2)nc1)C(=O)OCc1ccccc1 ; CN(Cc1cnc(N(C)c2ccc(N3CCN(CC(=O)Nc4ccc5c(c4)CN([C@H]4CCC(=O)NC4=O)C5=O)CC3)c(C(F)(F)F)c2)nc1)C(=O)OCc1ccccc1 ; Cl ; CNCc1cnc(N(C)c2ccc(N3CCN(CC(=O)Nc4ccc5c(c4)CN([C@H]4CCC(=O)NC4=O)C5=O)CC3)c(C(F)(F)F)c2)nc1]

Step 1: A solution of benzyl ((2-chloropyrimidin-5-yl)methyl)(methyl)carbamate (2.50 g, 8.60 mmol), tert-butyl 4-(4-(methylamino)-2-(trifluoromethyl)phenyl) piperazine-1-carboxylate (3.10 g, 8.60 mmol) and TFA (0.19 g, 1.70 mmol) in IPA (30 mL) was stirred at 80° C. for 12 hours. Then TFA (4.90 g, 43.96 mmol) was slowly added dropwise. The reaction was stirred 80° C. for 3 hours. The mixture was concentrated to give benzyl methyl((2-(methyl(4-(piperazin-1-yl)-3-(trifluoromethyl)phenyl)amino)pyrimidin-5-yl)methyl)carbamate (6-1b, 1.50 g, 34.20%). Use directly without purification. ESI-MS: m/z=515.1 [M+1]⁺.
Step 2: A solution of benzyl methyl((2-(methyl(4-(piperazin-1-yl)-3-(trifluoromethyl)phenyl)amino)pyrimidin-5-yl)methyl)carbamate (1.2 g, 2.3 mmol), benzyl methyl((2-(methyl(4-(piperazin-1-yl)-3-(trifluoromethyl)phenyl)amino) pyrimidin-5-yl)methyl)-carbamate (0.77 g, 2.30 mmol) and TEA (1.16 g, 11.50 mmol) in DMSO (20 mL) was stirred at 85° C. for 12 hours. The reaction mixture was poured into water (100 mL) and extracted with EtOAc (3×25 mL). The combined organic layers were washed with brine, dried over Na₂SO₄ and concentrated to afford a crude product. The crude product was purified by prep-TLC (PE/EtOAc=3/1) to give benzyl (S)-((2-((4-(4-(2-((2-(2,6-dioxopiperidin-3-yl)-1-oxoisoindolin-5-yl)amino)-2-oxoethyl)piperazin-1-yl)-3-(trifluoromethyl)phenyl)(methyl)amino)pyrimidin-5-yl)methyl)(methyl)carbamate (6-1c, 0.40 g, 21.80%) as a yellow solid. ESI-MS: m/z=814.3 [M+1]⁺.
Step 3: A solution of benzyl (S)-((2-((4-(4-(2-((2-(2,6-dioxopiperidin-3-yl)-1-oxoisoindolin-5-yl)amino)-2-oxoethyl)piperazin-1-yl)-3-(trifluoromethyl)phenyl) (methyl)amino)pyrimidin-5-yl)methyl)(methyl)carbamate (400.0 mg, 0.49 mmol) in HCl/EtOAc (10 mL) was stirred at 25° C. for 6 hours. The mixture was concentrated to afford a crude product. The residue was purified by prep-HPLC and lyophilized to give (S)—N-(2-(2,6-dioxopiperidin-3-yl)-1-oxoisoindolin-5-yl)-2-(4-(4-(methyl(5-((methylamino)methyl)pyrimidin-2-yl)amino)-2-(trifluoromethyl)phenyl)piperazin-1-yl)acetamide (6-1, 300 mg, 89.80%) as a white solid. ¹H NMR (400 MHz, MeOD) δ 2.14-2.22 (m, 1H), 2.45-2.54 (m, 1H), 2.75 (s, 3H), 2.79 (d, J=2.4 Hz, 1H), 2.84-2.96 (m, 1H), 3.31-3.36 (m, 2H), 3.38-3.54 (m, 4H), 3.62 (s, 3H), 3.83 (d, J=10.4 Hz, 2H), 4.19 (s, 2H), 4.36 (s, 2H), 4.49 (q, J=17.2 Hz, 2H), 5.14 (dd, J=13.2, 5.2 Hz, 1H), 7.69 (dd, J=8.4, 1.6 Hz, 1H), 7.72-7.82 (m, 4H), 8.04 (s, 1H), 8.64 (s, 2H). ESI-MS: m/z=680.3 [M+1]⁺.

### Synthesis of Cbz-Amine Intermediate

### Method 51

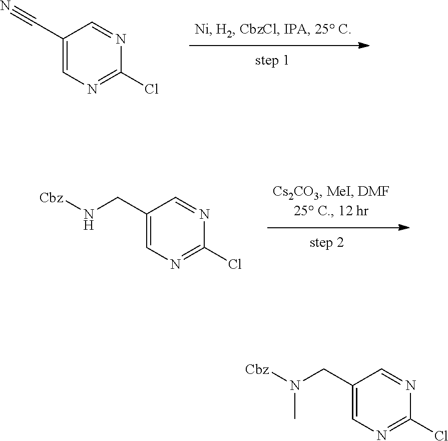 [SMILES: N#Cc1cnc(Cl)nc1 ; O=C(NCc1cnc(Cl)nc1)OCc1ccccc1 ; CN(Cc1cnc(Cl)nc1)C(=O)OCc1ccccc1]

Step 1: A solution of 2-chloropyrimidine-5-carbonitrile (25 g, 0.1792 mol), CbzCl (45.85 g, 0.2688 mol) and Raney Ni (26.3 g) in THF (200 mL) was stirred at 25° C. for 3 hours under H₂ (20 psi). The mixture was filtered. The filtrate was concentrated to afford a crude product. The crude product was purified by silica gel chromatography (PE/EtOAc=20/1˜3/1) to give benzyl ((2-chloropyrimidin-5-yl)methyl)carbamate (10.00 g, 20.10%) as a yellow solid. ESI-MS: m/z=277.9 [M+1]⁺.
Step 2: To a stirred solution of benzyl ((2-chloropyrimidin-5-yl)methyl)carbamate (10.00 g, 36.00 mmol) in DMF (120 mL) was added Cs₂CO₃ (23.46 g, 72.00 mmol) and CH₃I (6.13 g, 43.17 mmol) at 25° C. The reaction was stirred 25° C. for 12 hours. The reaction mixture was poured into water (300 mL) and extracted with EtOAc (3×50 mL). The combined organic layers were washed with brine, dried over Na₂SO₄ and concentrated to afford a crude product. The crude product was purified by silica gel chromatography (PE/EtOAc=20/1˜5/1) to give benzyl ((2-chloropyrimidin-5-yl)methyl)(methyl)carbamate (7.20 g, 68.54%) as a yellow solid.

### Synthesis of Compound 6-2a

### Method 52

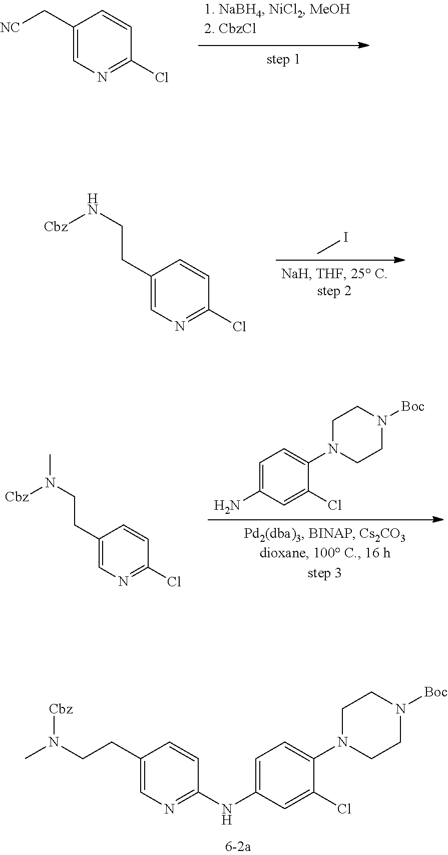

Step 1: To a stirred solution of 2-(6-chloropyridin-3-yl) acetonitrile (20.00 g, 0.13 mol) in MeOH (200 mL) stirred under nitrogen was added NiCl₂ (16.99 g, 0.13 mol) and CbzCl (44.73 g, 0.26 mol) dropwise at 25° C. Then NaBH₄ (14.88 g, 0.39 mol) was slowly added in batches at 0° C. The reaction was stirred at 25° C. for 2 hours. The reaction mixture was slowly poured into ice water (400 mL) and extracted with EtOAc (200 mL×3). The combined organic layers were washed with brine, dried over Na₂SO₄ and concentrated to afford a crude product. The crude product was purified by silica gel chromatography (PE/EtOAc=100/1˜2/1) to give benzyl (2-(6-chloropyridin-3-yl) ethyl) carbamate (15.00 g, 34.94% yield) as a white solid. ESI-MS: m/z=291.2 [M+1]⁺
Step 2: To a stirred solution of benzyl (2-(6-chloropyridin-3-yl) ethyl) carbamate (15.00 g, 0.05 mol) in THF (150 mL) stirred under nitrogen was added NaH (4.13 g, 0.10 mol, 60% in oil) at 0° C. The mixture was stirred at 25° C. for 0.5 hours. Then CH₃I (14.65 g, 0.10 mol) was slowly added dropwise. The reaction was stirred 25° C. for 2 hours. The reaction mixture was slowly poured into ice water (150 mL) and extracted with EtOAc (100 mL×3). The combined organic layers were washed with brine, dried over Na₂SO₄ and concentrated to give benzyl (2-(6-chloropyridin-3-yl) ethyl) (methyl)carbamate (7.00 g, 43.36% yield) as a yellow oil. ESI-MS: m/z=305.3 [M+1]⁺
Step 3: To a solution of benzyl (2-(6-chloropyridin-3-yl) ethyl) (methyl)carbamate (5.00 g, 16.4 mmol) in 1,4-dioxane (50 mL) stirred under nitrogen was added tert-butyl 4-(4-amino-2-chlorophenyl) piperazine-1-carboxylate (5.13 g, 16.4 mmol), Cs₂CO₃ (16.03 g, 49.2 mmol), Pd₂(dba)₃ (500 mg) and BINAP (500 mg). The reaction was stirred 100° C. for 16 hours. The reaction mixture was poured into water (100 mL) and extracted with EtOAc (30 mL×3). The combined organic layers were washed with brine, dried over Na₂SO₄ and concentrated to afford a crude product. The crude product was purified by silica gel chromatography (PE/EtOAc=100/1˜1/1) to give tert-butyl 4-(4-((5-(2-(((benzyloxy) carbonyl) (methyl) amino) ethyl) pyridin-2-yl) amino)-2-chlorophenyl) piperazine-1-carboxylate (6-2a, 2.00 g, 18.9900 yield) as a yellow solid. ESI-MS: m/z=580.3 [M+1]⁺

The following compounds 6-1 through 6-23 (23) shown in Table 6 were prepared by methods similar to those described for the preparation of 6-1, using the appropriate Boc-amine (19) and Glutarimide (21).

TABLE 6

| | | | Structure | |
|---|---|---|---|---|
| Compd | 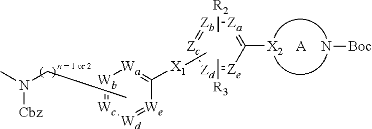 | 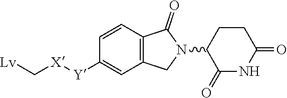 | 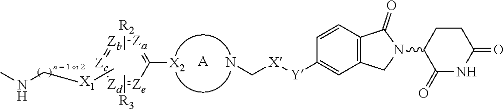 | MS found [M + H]+ |
| 6-1 | 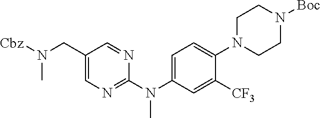 [SMILES: CN(Cc1cnc(N(C)c2ccc(N3CCN(C(=O)OC(C)(C)C)CC3)c(C(F)(F)F)c2)nc1)C(=O)OCc1ccccc1] | 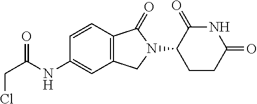 [SMILES: O=C1CC[C@H](N2Cc3cc(NC(=O)CCl)ccc3C2=O)C(=O)N1] | 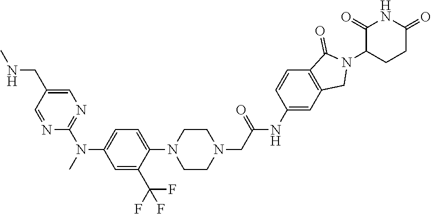 [SMILES: CNCc1cnc(N(C)c2ccc(N3CCN(CC(=O)Nc4ccc5c(c4)CN(C4CCC(=O)NC4=O)C5=O)CC3)c(C(F)(F)F)c2)nc1] | 588.4 |
| | | | ¹H NMR (400 MHz, MeOD) δ 2.14-2.22 (m, 1H), | |
| | | | 2.45-2.54 (m, 1H), 2.75 (s, 3H), 2.79 (d, J = 2.4 Hz, | |
| | | | 1H), 2.84-2.96 (m, 1H), 3.31-3.36 (m, 2H), 3.38- | |
| | | | 3.54 (m, 4H), 3.62 (s, 3H), 3.83 (d, J = 10.4 Hz, 2H), | |
| | | | 4.19 (s, 2H), 4.36 (s, 2H), 4.49 (q, J = 17.2 Hz, 2H), | |
| | | | 5.14 (dd, J = 13.2, 5.2 Hz, 1H), 7.69 (dd, J = 8.4, 1.6 | |
| | | | Hz, 1H), 7.72-7.82 (m, 4H), 8.04 (s, 1H), 8.64 (s, | |
| | | | 2H). | |
| 6-2 | 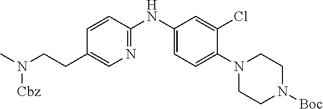 [SMILES: CN(CCc1ccc(Nc2ccc(N3CCN(C(=O)OC(C)(C)C)CC3)c(Cl)c2)nc1)C(=O)OCc1ccccc1] | 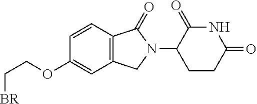 | 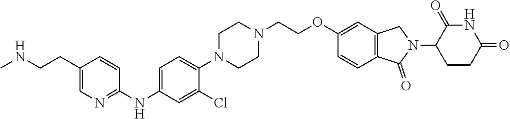 [SMILES: CNCCc1ccc(Nc2ccc(N3CCN(CCOc4ccc5c(c4)CN(C4CCC(=O)NC4=O)C5=O)CC3)c(Cl)c2)nc1] | 647.3, 649.3 |
| | | | ¹H NMR (400 MHz, MeOD_d4) 8 2.14-2.22 (m, | |
| | | | 1H), 2.43-2.55 (m, 1H), 2.66 (s, 3H), 2.76 (s, 2H), | |
| | | | 2.79-2.84 (m, 1H), 2.86-2.97 (m, 1H), 2.97-3.03 | |
| | | | (m, 2H), 3.27 (s, 2H), 3.45-3.65 (m, 4H), 3.79-3.87 | |
| | | | (m, 4H), 4.49 (q, J = 17.0 Hz, 2H), 4.56-4.60 (m, | |
| | | | 2H), 5.14 (dd, J = 13.4, 5.0 Hz, 1H), 7.20-7.24 (m, | |
| | | | 2H), 7.29 (d, J = 2.0 Hz, 1H), 7.39 (d, J = 1.2 Hz, 1H), | |
| | | | 7.56 (t, J = 1.2 Hz, 1H), 7.78 (d, J = 8.4 Hz, 1H), 7.88 | |
| | | | (d, J = 1.6 Hz, 1H), 8.04 (dd, J = 9.2, 2.2 Hz, 1H). | |
| 6-3 | 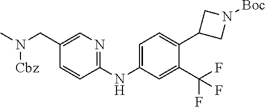 [SMILES: CN(Cc1ccc(Nc2ccc(C3CN(C(=O)OC(C)(C)C)C3)c(C(F)(F)F)c2)nc1)C(=O)OCc1ccccc1] | 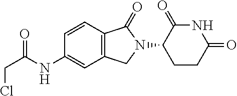 [SMILES: O=C1CC[C@H](N2Cc3cc(NC(=O)CCl)ccc3C2=O)C(=O)N1] | 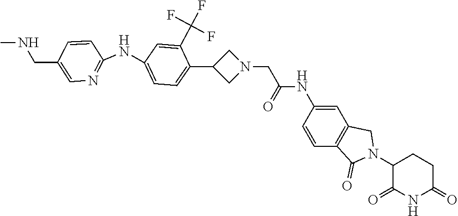 [SMILES: CNCc1ccc(Nc2ccc(C3CN(CC(=O)Nc4ccc5c(c4)CN(C4CCC(=O)NC4=O)C5=O)C3)c(C(F)(F)F)c2)nc1] | 635.4 |
| 6-4 | 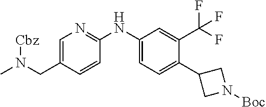 [SMILES: CN(Cc1ccc(Nc2ccc(C3CN(C(=O)OC(C)(C)C)C3)c(C(F)(F)F)c2)nc1)C(=O)OCc1ccccc1] | 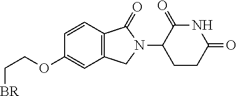 | 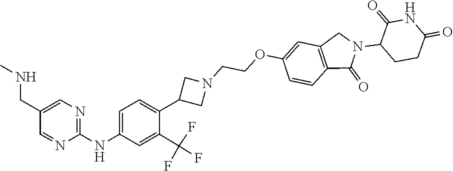 [SMILES: CNCc1cnc(Nc2ccc(C3CN(CCOc4ccc5c(c4)CN(C4CCC(=O)NC4=O)C5=O)C3)c(C(F)(F)F)c2)nc1] | 623.4, 312.2 |
| 6-5 | 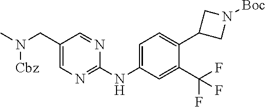 [SMILES: CN(Cc1cnc(Nc2ccc(C3CN(C(=O)OC(C)(C)C)C3)c(C(F)(F)F)c2)nc1)C(=O)OCc1ccccc1] | 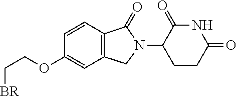 | 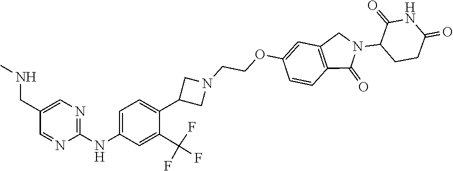 [SMILES: CNCc1cnc(Nc2ccc(C3CN(CCOc4ccc5c(c4)CN(C4CCC(=O)NC4=O)C5=O)C3)c(C(F)(F)F)c2)nc1] | 624.4, 312.5 |
| 6-6 | 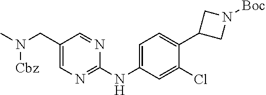 [SMILES: CN(Cc1cnc(Nc2ccc(C3CN(C(=O)OC(C)(C)C)C3)c(Cl)c2)nc1)C(=O)OCc1ccccc1] | 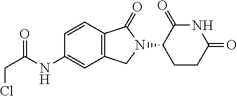 [SMILES: O=C1CC[C@H](N2Cc3cc(NC(=O)CCl)ccc3C2=O)C(=O)N1] | 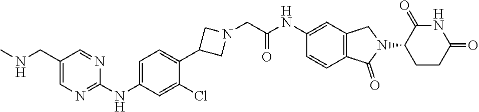 [SMILES: CNCc1cnc(Nc2ccc(C3CN(CC(=O)Nc4ccc5c(c4)CN([C@H]4CCC(=O)NC4=O)C5=O)C3)c(Cl)c2)nc1] | 603.3, 302.1 |
| 6-7 | 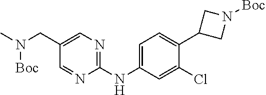 [SMILES: CN(Cc1cnc(Nc2ccc(C3CN(C(=O)OC(C)(C)C)C3)c(Cl)c2)nc1)C(=O)OC(C)(C)C] | 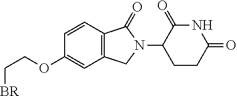 | 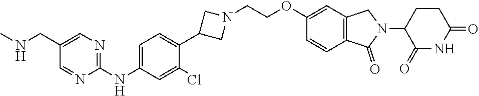 [SMILES: CNCc1cnc(Nc2ccc(C3CN(CCOc4ccc5c(c4)CN(C4CCC(=O)NC4=O)C5=O)C3)c(Cl)c2)nc1] | 590.3, 295.7 |
| 6-8 | 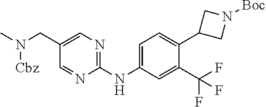 [SMILES: CN(Cc1cnc(Nc2ccc(C3CN(C(=O)OC(C)(C)C)C3)c(C(F)(F)F)c2)nc1)C(=O)OCc1ccccc1] | 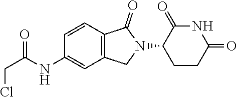 [SMILES: O=C1CC[C@H](N2Cc3cc(NC(=O)CCl)ccc3C2=O)C(=O)N1] | 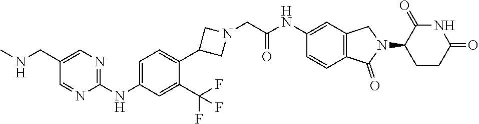 [SMILES: CNCc1cnc(Nc2ccc(C3CN(CC(=O)Nc4ccc5c(c4)CN([C@@H]4CCC(=O)NC4=O)C5=O)C3)c(C(F)(F)F)c2)nc1] | 637.4, 319.2 |
| 6-9 | 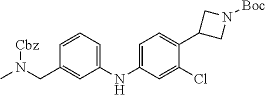 [SMILES: CN(Cc1cccc(Nc2ccc(C3CN(C(=O)OC(C)(C)C)C3)c(Cl)c2)c1)C(=O)OCc1ccccc1] | 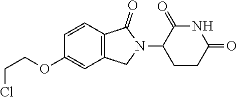 [SMILES: O=C1CCC(N2Cc3cc(OCCCl)ccc3C2=O)C(=O)N1] | 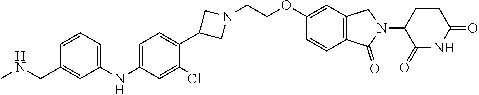 [SMILES: CNCc1cccc(Nc2ccc(C3CN(CCOc4ccc5c(c4)CN(C4CCC(=O)NC4=O)C5=O)C3)c(Cl)c2)c1] | 588.4, |
| 6-10 | 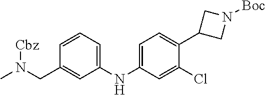 [SMILES: CN(Cc1cccc(Nc2ccc(C3CN(C(=O)OC(C)(C)C)C3)c(Cl)c2)c1)C(=O)OCc1ccccc1] | 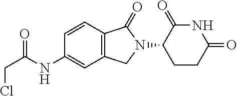 [SMILES: O=C1CC[C@H](N2Cc3cc(NC(=O)CCl)ccc3C2=O)C(=O)N1] | 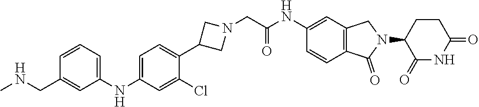 [SMILES: CNCc1cccc(Nc2ccc(C3CN(CC(=O)Nc4ccc5c(c4)CN([C@H]4CCC(=O)NC4=O)C5=O)C3)c(Cl)c2)c1] | 601.4, 301.5 |
| 6-11 | 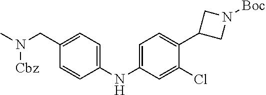 [SMILES: CN(Cc1ccc(Nc2ccc(C3CN(C(=O)OC(C)(C)C)C3)c(Cl)c2)cc1)C(=O)OCc1ccccc1] | 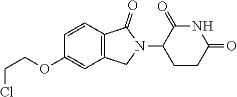 [SMILES: O=C1CCC(N2Cc3cc(OCCCl)ccc3C2=O)C(=O)N1] | 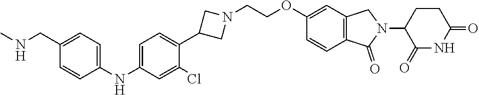 [SMILES: CNCc1ccc(Nc2ccc(C3CN(CCOc4ccc5c(c4)CN(C4CCC(=O)NC4=O)C5=O)C3)c(Cl)c2)cc1] | 588.4, 295.2 |
| 6-12 | 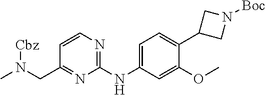 [SMILES: COc1cc(Nc2nccc(CN(C)C(=O)OCc3ccccc3)n2)ccc1C1CN(C(=O)OC(C)(C)C)C1] | 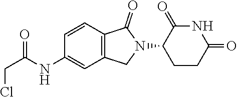 [SMILES: O=C1CC[C@H](N2Cc3cc(NC(=O)CCl)ccc3C2=O)C(=O)N1] | 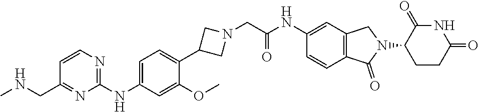 [SMILES: CNCc1ccnc(Nc2ccc(C3CN(CC(=O)Nc4ccc5c(c4)CN([C@H]4CCC(=O)NC4=O)C5=O)C3)c(OC)c2)n1] | 300.2, 599.3 |
| 6-13 | 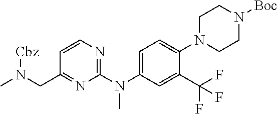 [SMILES: CN(Cc1ccnc(N(C)c2ccc(N3CCN(C(=O)OC(C)(C)C)CC3)c(C(F)(F)F)c2)n1)C(=O)OCc1ccccc1] | 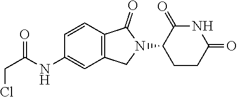 [SMILES: O=C1CC[C@H](N2Cc3cc(NC(=O)CCl)ccc3C2=O)C(=O)N1] | 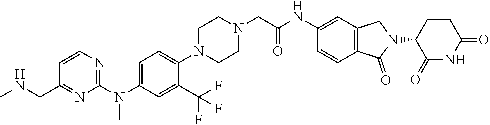 [SMILES: CNCc1ccnc(N(C)c2ccc(N3CCN(CC(=O)Nc4ccc5c(c4)CN([C@@H]4CCC(=O)NC4=O)C5=O)CC3)c(C(F)(F)F)c2)n1] | 340.6, 680.2 |
| 6-14 | 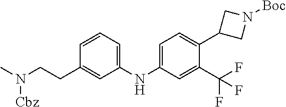 [SMILES: CN(CCc1cccc(Nc2ccc(C3CN(C(=O)OC(C)(C)C)C3)c(C(F)(F)F)c2)c1)C(=O)OCc1ccccc1] | 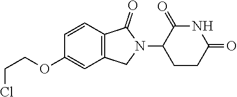 [SMILES: O=C1CCC(N2Cc3cc(OCCCl)ccc3C2=O)C(=O)N1] | 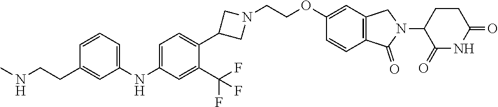 [SMILES: CNCCc1cccc(Nc2ccc(C3CN(CCOc4ccc5c(c4)CN(C4CCC(=O)NC4=O)C5=O)C3)c(C(F)(F)F)c2)c1] | 636.3, 318.8 |
| 6-15 | 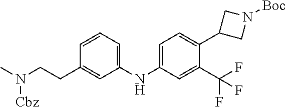 [SMILES: CN(CCc1cccc(Nc2ccc(C3CN(C(=O)OC(C)(C)C)C3)c(C(F)(F)F)c2)c1)C(=O)OCc1ccccc1] | 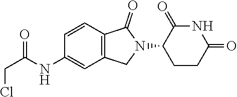 [SMILES: O=C1CC[C@H](N2Cc3cc(NC(=O)CCl)ccc3C2=O)C(=O)N1] | 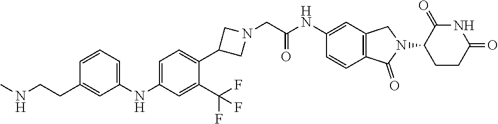 [SMILES: CNCCc1cccc(Nc2ccc(C3CN(CC(=O)Nc4ccc5c(c4)CN([C@H]4CCC(=O)NC4=O)C5=O)C3)c(C(F)(F)F)c2)c1] | 649.4, 325.3 |
| 6-16 | 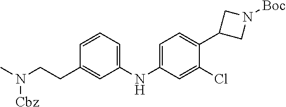 [SMILES: CN(CCc1cccc(Nc2ccc(C3CN(C(=O)OC(C)(C)C)C3)c(Cl)c2)c1)C(=O)OCc1ccccc1] | 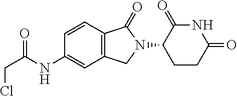 [SMILES: O=C1CC[C@H](N2Cc3cc(NC(=O)CCl)ccc3C2=O)C(=O)N1] | 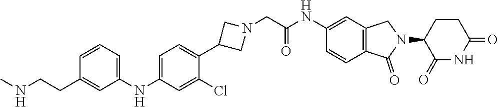 [SMILES: CNCCc1cccc(Nc2ccc(C3CN(CC(=O)Nc4ccc5c(c4)CN([C@H]4CCC(=O)NC4=O)C5=O)C3)c(Cl)c2)c1] | 615.3, 307.9 |
| 6-17 | 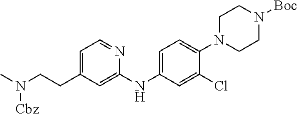 [SMILES: CN(CCc1ccnc(Nc2ccc(N3CCN(C(=O)OC(C)(C)C)CC3)c(Cl)c2)c1)C(=O)OCc1ccccc1] | 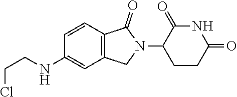 [SMILES: O=C1CCC(N2Cc3cc(NCCCl)ccc3C2=O)C(=O)N1] | 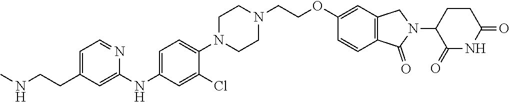 [SMILES: CNCCc1ccnc(Nc2ccc(N3CCN(CCOc4ccc5c(c4)CN(C4CCC(=O)NC4=O)C5=O)CC3)c(Cl)c2)c1] | 316.8, 633.3 |
| 6-18 | 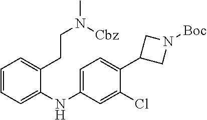 [SMILES: CN(CCc1ccccc1Nc1ccc(C2CN(C(=O)OC(C)(C)C)C2)c(Cl)c1)C(=O)OCc1ccccc1] | 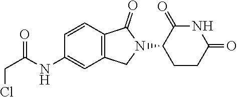 [SMILES: O=C1CC[C@H](N2Cc3cc(NC(=O)CCl)ccc3C2=O)C(=O)N1] | 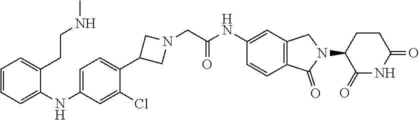 [SMILES: CNCCc1ccccc1Nc1ccc(C2CN(CC(=O)Nc3ccc4c(c3)CN([C@H]3CCC(=O)NC3=O)C4=O)C2)c(Cl)c1] | 615.5, 308.1 |
| 6-19 | 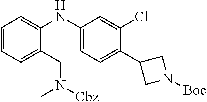 [SMILES: CN(Cc1ccccc1Nc1ccc(C2CN(C(=O)OC(C)(C)C)C2)c(Cl)c1)C(=O)OCc1ccccc1] | 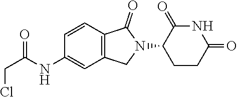 [SMILES: O=C1CC[C@H](N2Cc3cc(NC(=O)CCl)ccc3C2=O)C(=O)N1] | 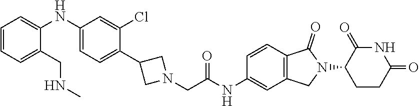 [SMILES: CNCc1ccccc1Nc1ccc(C2CN(CC(=O)Nc3ccc4c(c3)CN([C@H]3CCC(=O)NC3=O)C4=O)C2)c(Cl)c1] | 601.3, 301.5 |
| 6-20 | 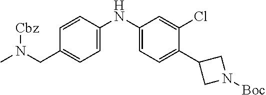 [SMILES: CN(Cc1ccc(Nc2ccc(C3CN(C(=O)OC(C)(C)C)C3)c(Cl)c2)cc1)C(=O)OCc1ccccc1] | 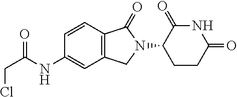 [SMILES: O=C1CC[C@H](N2Cc3cc(NC(=O)CCl)ccc3C2=O)C(=O)N1] | 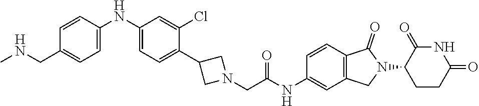 [SMILES: CNCc1ccc(Nc2ccc(C3CN(CC(=O)Nc4ccc5c(c4)CN([C@H]4CCC(=O)NC4=O)C5=O)C3)c(Cl)c2)cc1] | 601.4, 301.3 |
| 6-21 | 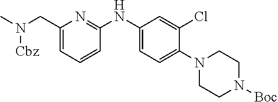 [SMILES: CN(Cc1cccc(Nc2ccc(N3CCN(C(=O)OC(C)(C)C)CC3)c(Cl)c2)n1)C(=O)OCc1ccccc1] | 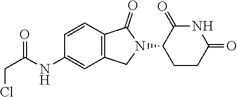 [SMILES: O=C1CC[C@H](N2Cc3cc(NC(=O)CCl)ccc3C2=O)C(=O)N1] | 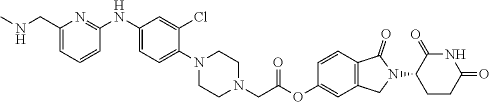 [SMILES: CNCc1cccc(Nc2ccc(N3CCN(CC(=O)Oc4ccc5c(c4)CN([C@H]4CCC(=O)NC4=O)C5=O)CC3)c(Cl)c2)n1] | 631.4, 633.5 |
| 6-22 | 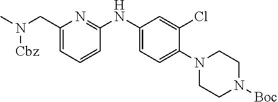 [SMILES: CN(Cc1cccc(Nc2ccc(N3CCN(C(=O)OC(C)(C)C)CC3)c(Cl)c2)n1)C(=O)OCc1ccccc1] | 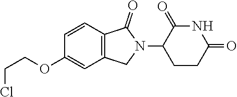 [SMILES: O=C1CCC(N2Cc3cc(OCCCl)ccc3C2=O)C(=O)N1] | 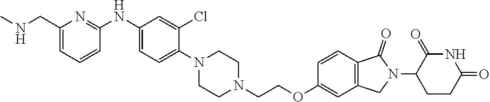 [SMILES: CNCc1cccc(Nc2ccc(N3CCN(CCOc4ccc5c(c4)CN(C4CCC(=O)NC4=O)C5=O)CC3)c(Cl)c2)n1] | 649.2, 651.3 |
| 6-23 | 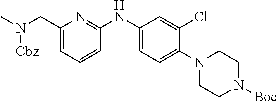 [SMILES: CN(Cc1cccc(Nc2ccc(N3CCN(C(=O)OC(C)(C)C)CC3)c(Cl)c2)n1)C(=O)OCc1ccccc1] | 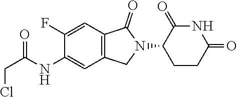 [SMILES: O=C1CC[C@H](N2Cc3cc(NC(=O)CCl)c(F)cc3C2=O)C(=O)N1] | 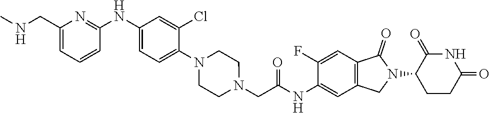 [SMILES: CNCc1cccc(Nc2ccc(N3CCN(CC(=O)Nc4cc5c(cc4F)C(=O)N([C@H]4CCC(=O)NC4=O)C5)CC3)c(Cl)c2)n1] | 649.2, 651.3 |

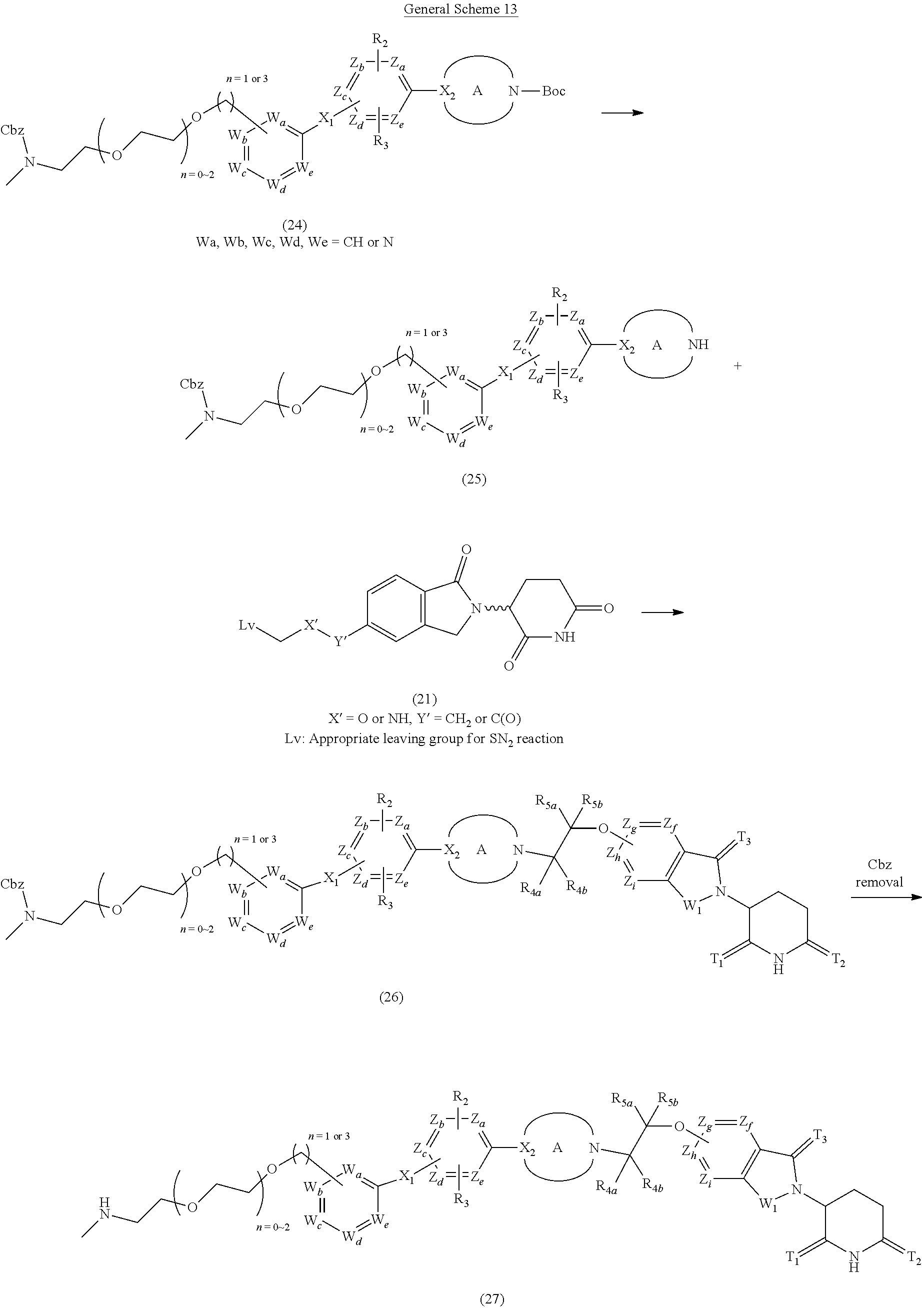

### Method 53

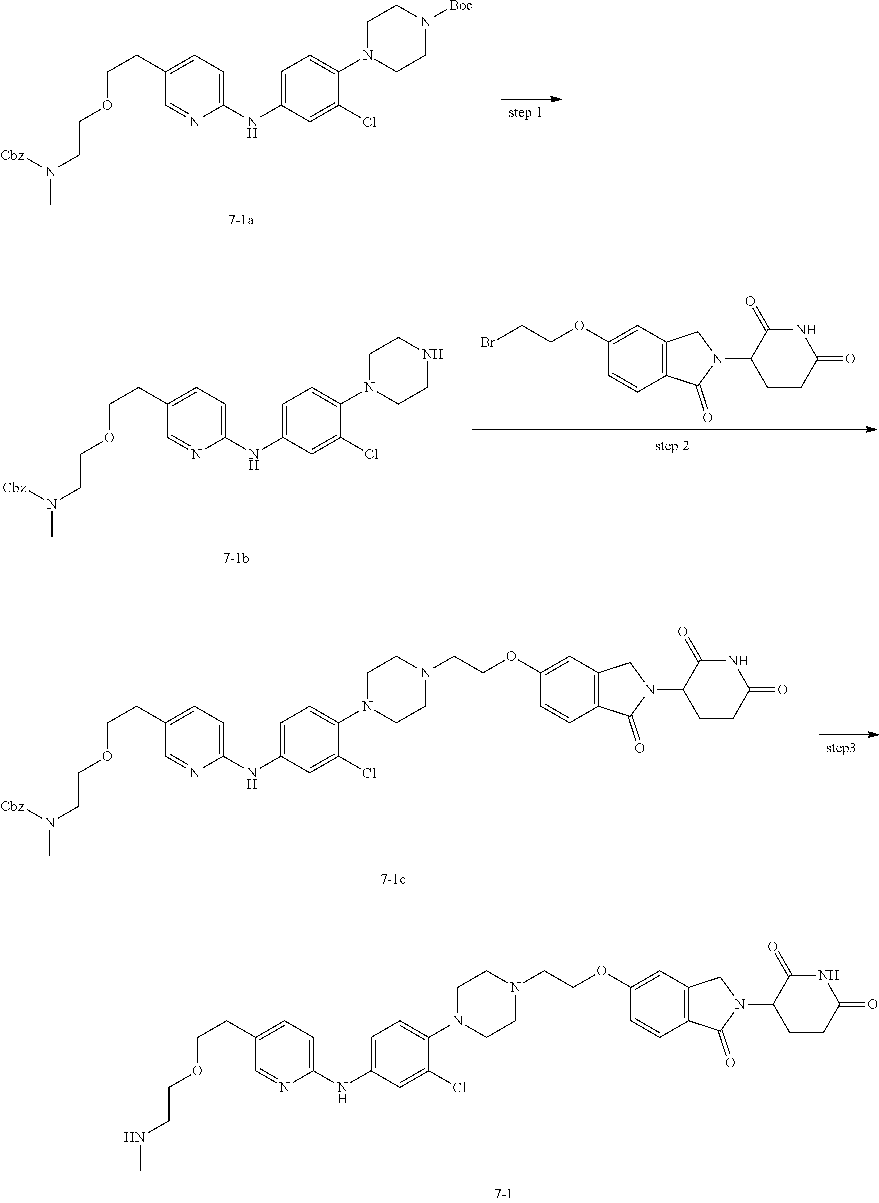 [SMILES: CN(CCOCCc1ccc(Nc2ccc(N3CCN(C(=O)OC(C)(C)C)CC3)c(Cl)c2)nc1)C(=O)OCc1ccccc1 ; CN(CCOCCc1ccc(Nc2ccc(N3CCNCC3)c(Cl)c2)nc1)C(=O)OCc1ccccc1 ; O=C1CCC(N2Cc3cc(OCCBr)ccc3C2=O)C(=O)N1 ; CN(CCOCCc1ccc(Nc2ccc(N3CCN(CCOc4ccc5c(c4)CN(C4CCC(=O)NC4=O)C5=O)CC3)c(Cl)c2)nc1)C(=O)OCc1ccccc1 ; CNCCOCCc1ccc(Nc2ccc(N3CCN(CCOc4ccc5c(c4)CN(C4CCC(=O)NC4=O)C5=O)CC3)c(Cl)c2)nc1]

Step 1: To a solution of tert-butyl 4-(4-((5-(2-(2-(((benzyloxy)carbonyl)(methyl) amino)ethoxy)ethyl)pyridin-2-yl)amino)-2 chlorophenyl)piperazine-1-carboxylate (7-1a, 500 mg, 0.80 mmol) in DCM (5 mL) was added TFA (1 mL). The reaction mixture was stirred at 25° C. for 5 hours. The reaction mixture was concentrated to give benzyl (2-(2-(6-((3-chloro-4-(piperazin-1-yl)phenyl)amino)pyridin-3-yl)ethoxy)ethyl)(methyl)carbamate (7-1b, 300 mg, 71.46%) as a brown oil. ESI-MS: m/z=524.1 [M+1]⁺.
Step 2: A solution of benzyl (2-(2-(6-((3-chloro-4-(piperazin-1-yl) phenyl) amino) pyridin-3-yl) ethoxy) ethyl)(methyl)carbamate (300 mg, 0.57 mmol), 3-(5-(2-bromoethoxy)-1-oxoisoindolin-2-yl)piperidine-2,6-dione (209.9 mg, 0.57 mmol) and TEA (174.4 mg, 1.71 mmol) in DMSO (5 mL) was stirred at 80° C. for 5 hours. The reaction mixture was poured into water (10 mL) and extracted with EtOAc (5 mL×3). The combined organic layers were washed with brine, dried over Na₂SO₄ and concentrated to afford a crude product. The crude product was purified by silica gel chromatography (PE/EtOAc=100/1˜5/1) to give benzyl (2-(2-(6-((3-chloro-4-(4-(2-((2-(2,6-dioxopiperidin-3-yl)-1-oxoisoindolin-5-yl)oxy)ethyl)piperazin-1-yl)phenyl)amino)pyridin-3-yl)ethoxy)ethyl)(methyl)carbamate (7-1c, 120 mg, 25.86%) as a white solid. ESI-MS: m/z=810.3 [M+1]⁺.
Step 3: A solution of benzyl (2-(2-(6-((3-chloro-4-(4-(2-((2-(2,6-dioxopiperidin-3-yl)-1-oxoisoindolin-5-yl)oxy)ethyl)piperazin-1-yl)phenyl)amino)pyridin-3-yl)ethoxy)ethyl)(methyl)carbamate (135 mg, 0.17 mmol) in 1,4-dioxane/HCl (4 mol/L, 3 mL) was stirred at 25° C. for 16 hours. The mixture was filtered. The filter cake was dried to dryness to give 3-(5-(2-(4-(2-chloro-4-((5-(2-(2-(methylamino)ethoxy)ethyl)pyridin-2-yl)amino)phenyl)piperazin-1-yl)ethoxy)-1-oxoisoindolin-2-yl)piperidine-2,6-dione (7-1, 70 mg, 61.9%) as a gray solid. ¹H NMR (400 MHz, DMSO_d₆) δ 2.12-2.27 (m, 1H), 2.41-2.58 (m, 1H), 2.73 (s, 3H), 2.75-2.83 (m, 1H), 2.84-2.95 (m, 3H), 3.15-3.24 (m, 2H), 3.34 (d, J=3.2 Hz, 2H), 3.46-3.58 (m, 3H), 3.58-3.67 (m, 2H), 3.70-3.88 (m, 8H), 4.42-4.53 (m, 2H), 4.54-4.65 (m, 2H), 5.07-5.18 (m, 1H), 7.25-7.15 (m, 2H), 7.29 (s, 1H), 7.34-7.43 (m, 2H), 7.53 (d, J=1.7 Hz, 1H), 7.78 (d, J=8.5 Hz, 1H), 7.84 (s, 1H), 8.08 (dd, J=9.1, 1.9 Hz, 1H). ESI-MS: m/z=676.3 [M+1]⁺.

### Synthesis of Diarylamine Intermediate

### Method 54

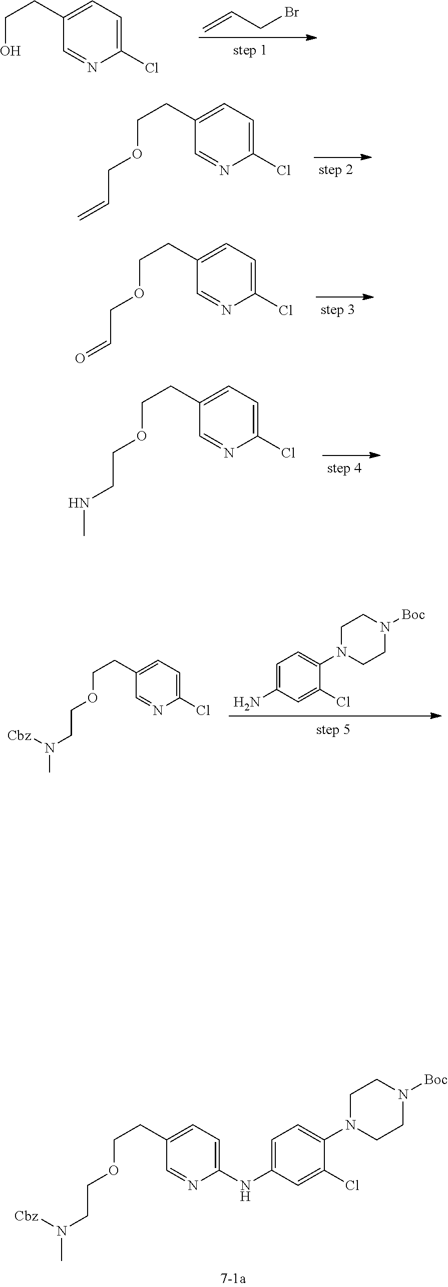

Step 1: To a stirred solution of 2-(6-chloropyridin-3-yl) ethan-1-ol (1.0 g, 6.37 mmol) in DMF (10 mL) was added NaH (0.77 g, 19.11 mmol) dropwise at 0° C. The mixture was stirred at 0° C. for 1 hour. Then 3-bromoprop-1-ene (1.15 g, 9.86 mmol) in DMF (2 mL) was slowly added dropwise. The reaction was stirred 25° C. for 2 hours. The reaction mixture was poured into water (50 mL) and extracted with EtOAc (20 mL×3). The combined organic layers were washed with brine, dried over Na₂SO₄ and concentrated to afford a crude product. The crude product was purified by prep-TLC (PE/EtOAc=3/1) to give 5-(2-(allyloxy)ethyl)-2-chloropyridine (0.30 g, 24.0%) as a white oil. ¹H NMR (400 MHz, DMSO_d₆) δ 2.84 (t, J=6.4 Hz, 2H), 3.59 (t, J=6.5 Hz, 2H), 3.93 (d, J=5.4 Hz, 2H), 5.11 (dd, J=10.4, 1.4 Hz, 1H), 5.19 (dd, J=17.4, 1.6 Hz, 1H), 5.75-5.90 (m, 1H), 7.43 (d, J=8.2 Hz, 1H), 7.71-7.77 (m, 1H), 8.29 (d, J=2.4). ESI-MS: m/z=198.1 [M+1]⁺.
Step 2: To a stirred solution of 5-(2-(allyloxy) ethyl)-2-chloropyridine (0.30 g, 1.52 mmol) in THF:H₂O=3:1 (5 mL) was added Potassium osmate (VI) dihydrate (46.70 mg, 0.15 mmol) in water (1 mL) dropwise at 0° C. The mixture was stirred at 0° C. for 15 minutes. Then Sodium periodate (0.99 g, 4.59 mmol) in THF (2 mL) was slowly added dropwise. The reaction was stirred at 25° C. for 2 hours. The reaction mixture was poured into water (20 mL) and extracted with EtOAc (10 mL×3). The combined organic layers were washed with brine, dried over Na₂SO₄ and concentrated to afford a crude product. The crude product was purified by silica gel chromatography (PE/EtOAc=50/1˜10/1) to give 2-(2-(6-chloropyridin-3-yl)ethoxy)acetaldehyde (50.0 mg, 16.50%) as a white oil. ESI-MS: m/z=218.1 [M+1]⁺.
Step 3: To a stirred solution of 2-(2-(6-chloropyridin-3-yl)ethoxy)acetaldehyde (0.30 g, 0.51 mmol) in MeOH (3 mL) was added MeNH₂·HCl (104.04 mg, 1.53 mmol) at 25° C. The mixture was stirred at 25° C. for 16 hours. Then NaBH₃CN (64.26 mg, 1.02 mmol) was slowly added. The reaction was stirred 25° C. for 2 hours. The reaction mixture was poured into water (10 mL) and extracted with EtOAc (5 mL×3). The combined organic layers were washed with brine, dried over Na₂SO₄ and concentrated to afford a crude product. The crude product was purified by prep-TLC (PE/EtOAc=3/1) to give 2-(2-(6-chloropyridin-3-yl)ethoxy)-N-methylethan-1-amine (0.20 g, 61.99%) as a white oil. ESI-MS: m/z=215.3 [M+19]⁺
Step 4: A solution of 2-(2-(6-chloropyridin-3-yl)ethoxy)-N-methylethan-1-amine (0.15 g, 0.71 mmol), CbzCl (0.18 g, 1.05 mmol) and TEA (0.22 g, 2.13 mmol) in DCM (3 mL) was stirred at 25° C. for 3 hours. The reaction mixture was concentrated to afford a crude product. The residue was stirred in MTBE (5 mL) for 30 minutes. The mixture was filtered, and the filter cake was dried to dryness to give benzyl (2-(2-(6-chloropyridin-3-yl) ethoxy) ethyl) (methyl)carbamate (20 mg, 8.20%) as a white oil. ¹H NMR (400 MHz, DMSO_d₆) δ2.75-2.85 (m, 5H), 3.33-3.40 (m, 2H), 3.44-3.52 (m, 2H), 3.54-3.62 (m, 2H), 5.05 (d, J=3.2 Hz, 2H), 7.22-7.43 (m, 5H), 7.75-7.64 (m, 1H), 8.26 (d, J=3.7 Hz, 1H). ESI-MS: m/z=349.1 [M+1]⁺.
Step 5: To a solution of benzyl (2-(2-(6-chloropyridin-3-yl) ethoxy) ethyl) (methyl)carbamate (100 mg, 0.29 mmol) in 1,4-dioxane (3 mL) was added Cs₂CO₃ (281.04 mg, 0.86 mmol), Pd₂(dba)₃ (10 mg), BINAP (10 mg) and tert-butyl 4-(4-amino-2-chlorophenyl)piperazine-1-carboxylate (90.2 mg, 0.29 mmol). The reaction mixture was stirred at 100° C. for 16 hours under N₂. The reaction mixture was poured into water (10 mL) and extracted with EtOAc (5 mL×3). The combined organic layers were washed with brine, dried over Na₂SO₄ and concentrated to afford a crude product. The crude product was purified by prep-TLC (PE/EtOAc=3/1) to give tert-butyl 4-(4-((5-(2-(2-(((benzyloxy)carbonyl)(methyl)amino)qethoxy) ethyl)pyridin-2-yl)amino)-2 chlorophenyl)piperazine-1-carboxylate (7-1a, 80 mg, 44.71%) as a yellow oil. ESI-MS: m/z=624.3 [M+1]⁺.

### Method 55

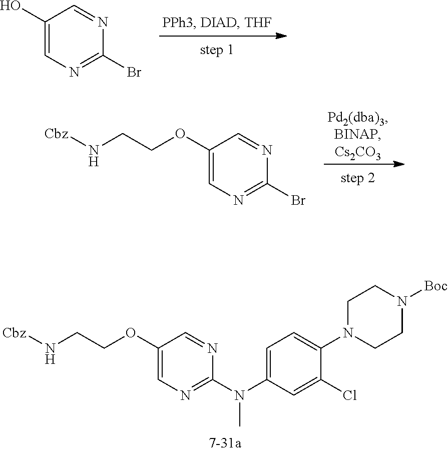 [SMILES: Oc1cnc(Br)nc1 ; O=C(NCCOc1cnc(Br)nc1)OCc1ccccc1 ; CN(c1ccc(N2CCN(C(=O)OC(C)(C)C)CC2)c(Cl)c1)c1ncc(OCCNC(=O)OCc2ccccc2)cn1]

Step 1: To a stirred solution of 2-bromopyrimidin-5-ol (2 g, 11.53 mmol) in THF (20 mL) was added benzyl (2-hydroxyethyl) carbamate (2.2 g, 11.53 mmol) and PPh₃ (4.5 g, 17.30 mmol) dropwise at 0° C. The mixture was stirred at 0° C. for 1 hour. Then DIAD (3.50 g, 17.30 mmol) in THF (40 mL) was slowly added dropwise. The reaction was stirred 25° C. for 2 hours. The reaction mixture was poured into water (200 mL) and extracted with EtOAc (100 mL×3). The combined organic layers were washed with brine, dried over Na₂SO₄ and concentrated to afford a crude product. The crude product was purified by silica gel chromatography (PE/EtOAc=100/1˜5/1) to benzyl (2-(2-bromophenoxy) ethyl) (methyl)carbamate (2 g, 49.69% yield) as a white oil.
Step 2: To a solution of benzyl (2-(2-bromophenoxy) ethyl) (methyl)carbamate (1.9 g, 5.4 mmol) in 1,4-dioxane (20 mL) was added Cs₂CO₃ (5.3 g, 16.2 mmol), Pd₂(dba)₃ (200 mg), BINAP (200 mg) and tert-butyl 4-(2-chloro-4-(methylamino)phenyl)piperazine-1-carboxylate (1.76 g, 5.4 mmol). The reaction mixture was stirred at 100° C. for 16 hours under N₂. The reaction mixture was poured into water (60 mL) and extracted with EtOAc (30 mL×3). The combined organic layers were washed with brine, dried over Na₂SO₄ and concentrated to afford a crude product. The crude product was purified by prep-TLC (PE/EtOAc=3/1) to give the tert-butyl 4-(4-((5-(2-(((benzyloxy)carbonyl)amino)ethoxy)pyrimidin-2-yl)(methyl)amino)-2-chlorophenyl)piperazine-1-carboxylate (7-31a, 0.76 g, 23.59% yield) as a yellow solid. ¹H NMR (400 MHz, DMSO_d₆) δ1.43 (s, 9H), 2.86-2.95 (m, 4H), 3.28-3.37 (m, 4H), 3.43-3.51 (m, 4H), 3.96-4.02 (m, 2H), 5.02 (s, 2H), 7.14 (d, J=8.8 Hz, 1H), 7.24 (dd, J=8.6, 2.6 Hz, 1H), 7.28-7.37 (m, 6H), 7.38-7.41 (m, 1H), 7.46-7.52 (m, 1H), 8.21 (s, 2H).

### Method 56

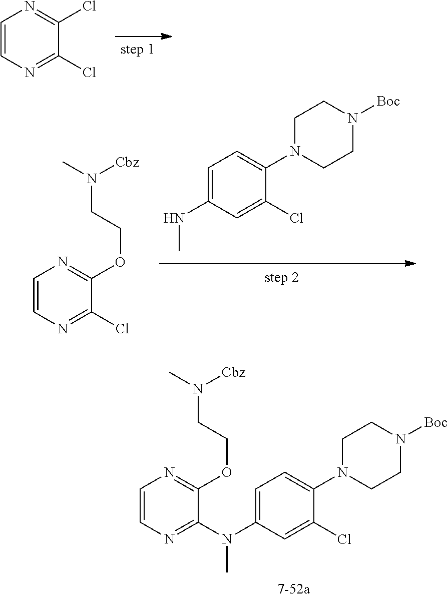 [SMILES: Clc1nccnc1Cl ; CN(CCOc1nccnc1Cl)C(=O)OCc1ccccc1 ; CNc1ccc(N2CCN(C(=O)OC(C)(C)C)CC2)c(Cl)c1 ; CN(CCOc1nccnc1N(C)c1ccc(N2CCN(C(=O)OC(C)(C)C)CC2)c(Cl)c1)C(=O)OCc1ccccc1]

Step 1: To a stirred solution of 2,3-dichloropyrazine (1.00 g, 6.7 mmol) in DMSO (10 mL) was added benzyl (2-hydroxyethyl) (methyl)carbamate (1.40 g, 6.7 mmol) and t-BuOK (1.50 g, 13.4 mmol) at 25° C. The reaction was stirred 120° C. for 16 hours. The reaction mixture was poured into water (30 mL) and extracted with EtOAc (30 mL×3). The combined organic layers were washed with brine, dried over Na₂SO₄ and concentrated to afford a crude product. The crude product was purified by silica gel chromatography (PE/EtOAc=100/1˜5/1) to give benzyl (2-((3-chloropyrazin-2-yl) oxy) ethyl) (methyl)carbamate (0.64 g, 11.47% yield) as a yellow solid. ESI-MS: m/z=321.9 [M+1]⁺
Step 2: To a solution of benzyl (2-((3-chloropyrazin-2-yl) oxy) ethyl) (methyl)carbamate (591 mg, 1.84 mmol) and tert-butyl 4-(2-chloro-4-(methylamino) phenyl) piperazine-1-carboxylate (600 mg, 1.84 mmol) in dioxane (8 mL) stirred under nitrogen was added Pd₂(dba)₃ (60 mg), BINAP (60 mg) and Cs₂CO₃ (1.79 g, 5.51 mmol). The reaction was stirred at 110° C. for 16 hours. The reaction mixture was poured into water (30 mL) and extracted with EtOAc (30 mL×3). The combined organic layers were washed with brine, dried over Na₂SO₄ and concentrated to afford a crude product. The crude product was purified by silica gel chromatography (PE/EtOAc=100/1˜3/1) to give tert-butyl 4-(4-((3-(2-(((benzyloxy) carbonyl) (methyl) amino) ethoxy) pyrazin-2-yl) (methyl)amino)-2-chlorophenyl) piperazine-1-carboxylate (7-52a, 210.0 mg, 18.53% yield) as a yellow solid. ESI-MS: m/z=611.0 [M+1]⁺

### Method 57

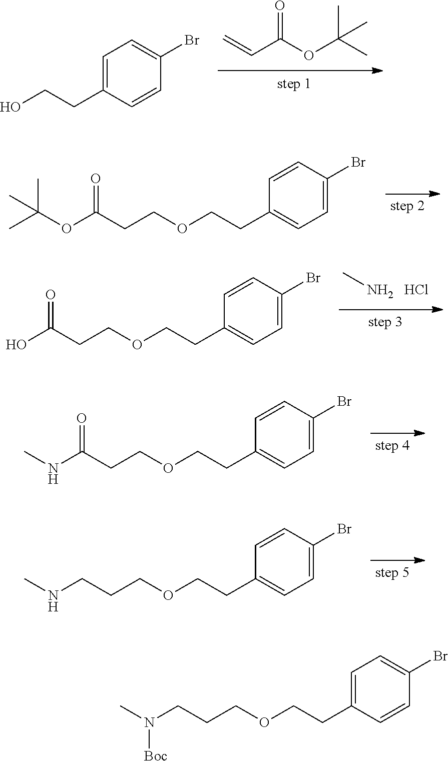

Step 1: To a solution of 2-(4-bromophenyl) ethan-1-ol (5.00 g, 0.02 mol) in Triton B (1 mL) stirred at 25° C. for 0.5 hours, the temperature was reduced to 0° C., and tert-butyl prop-2-enoate (3.51 g, 0.03 mol) was added. The reaction mixture was stirred at 25° C. for 15.5 hours. The reaction mixture was poured into ice water (200 mL) and extracted with EtOAc (150 mL×3). The combined organic layers were washed with brine, dried over Na₂SO₄ and concentrated to afford a crude product. The crude product was purified by silica gel chromatography (PE/EtOAc=20/1˜5/1) to give tert-butyl 3-(4-bromophenethoxy) propanoate (7.00 g, 85.50% yield) as a yellow oil. ¹H NMR (400 MHz, DMSO_d₆) δ 1.36 (s, 9H), 2.39 (t, J=6.4 Hz, 2H), 2.76 (t, J=6.4 Hz, 2H), 3.57 (t, J=6.4 Hz, 4H), 7.19 (d, J=8.0 Hz, 2H), 7.41-7.46 (m, 2H).
Step 2: To a solution of tert-butyl 3-(4-bromophenethoxy) propanoate (7.70 g, 0.02 mol) in DCM (60 mL) and TFA (12 mL). The reaction mixture was stirred at 25° C. for 16 hours. The reaction mixture was concentrated to give 3-(4-bromophenethoxy) propanoic acid (6.00 g, crude) as a yellow oil. ESI-MS: m/z=295.1 [M+23]⁺/297.1 [M+23]⁺
Step 3: A mixture of 3-(4-bromophenethoxy) propanoic acid (7.47 g, 0.03 mol), HATU (15.63 g, 0.04 mol), DIEA (17.71 g, 0.14 mol) and methylamine hydrochloride (9.25 g, 0.14 mol) in DCM (100 mL) was stirred at 25° C. for 16 hours. The reaction mixture was poured into water (200 mL) and extracted with DCM (150 mL×3). The combined organic layers were washed with brine, dried over Na₂SO₄ and concentrated to afford a crude product. The crude product was purified by silica gel chromatography (PE/EtOAc=20/1˜0/1) to give 3-(4-bromophenethoxy)-N-methylpropanamide (6.00 g, 79.88% yield) as a yellow solid. ¹H NMR (400 MHz, DMSO_d₆) δ2.28 (t, J=6.4 Hz, 2H), 2.76 (t, J=6.8 Hz, 2H), 3.18 (s, 4H), 3.54-3.60 (m, 4H), 7.20 (d, J=8.0 Hz, 2H), 7.42-7.47 (m, 2H).
Step 4: To a solution of 3-(4-bromophenethoxy)-N-methylpropanamide (5.00 g, 0.02 mol) in THF (500 mL) stirred at 0° C. was added BH₃-THF (40 mL, 0.09 mol, 2 mol/L). The reaction mixture was stirred at 60° C. for 3 hours. The reaction mixture was poured into ice water (200 mL) and extracted with EtOAc (150 mL×3). The combined organic layers were washed with brine, dried over Na₂SO₄ and concentrated to afford a crude product. The crude product was purified by silica gel chromatography (PE/EtOAc=20/1˜1/1) to give 3-(4-bromophenethoxy)-N-methylpropan-1-amine (4.00 g, crude) as a yellow oil. ESI-MS: m/z=272.0 [M+1]⁺/274.0 [M+1]⁺
Step 5: To a solution of 3-(4-bromophenethoxy)-N-methylpropan-1-amine (4.50 g, 0.02 mol), (Boc)₂O (5.40 g, 0.02 mol) in DCM (50 mL) stirred at 0° C. was added TEA (5.01 g, 0.05 mol). The reaction mixture was stirred at 25° C. for 16 hours. The reaction mixture was concentrated to afford a crude product. The residue was purified by prep-HPLC and lyophilized to give tert-butyl (3-(4-bromophenethoxy) propyl) (methyl)carbamate (2.00 g, 54.55% yield) as a yellow oil. ESI-MS: m/z=394.2/396.2 [M+23]⁺, 272.1/274.1 [M−100+1^{]+. 1}H NMR (400 MHz, DMSO_d₆) δ 1.37 (s, 9H), 1.59-1.71 (m, 2H), 2.71 (s, 3H), 2.77 (t, J=6.8 Hz, 2H), 3.14 (t, J=7.2 Hz, 2H), 3.34 (dd, J=12.8, 6.4 Hz, 2H), 3.55 (t, J=6.8 Hz, 2H), 7.20 (d, J=8.0 Hz, 2H), 7.41-7.51 (m, 2H).

### Method 58

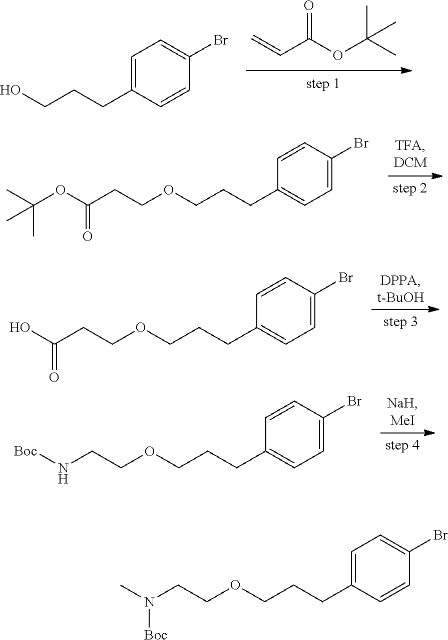

Step 1: To a stirred solution of 3-(4-bromophenyl) propan-1-ol (20 g, 0.09 mol) and tert-butyl acrylate (35.76 g, 0.27 mol) in THF (200 mL) was added Na (0.21 g, 9.3 mmol). The reaction mixture was stirred at 25° C. for 16 hours. The reaction mixture was slowly poured into ice water (200 mL) and extracted with EtOAc (200 mL×3). The combined organic layers were washed with brine, dried over Na₂SO₄ and concentrated to afford a crude product. The crude product was purified by silica gel chromatography (PE/EtOAc=100/1˜20/1) to give tert-butyl 3-(3-(4-bromophenyl) propoxy) propanoate (18.00 g, 52.11% yield) as a colorless oil. ESI-MS: m/z=343.0 [M+1]⁺, 345.0 [M+3]⁺
Step 2: To a solution of tert-butyl 3-(3-(4-bromophenyl) propoxy) propanoate (18 g, 0.05 mol) in DCM (180 mL) stirred was added TFA (18 mL) dropwise. The reaction mixture was stirred at 25° C. for 2 hours. The reaction mixture was concentrated to give 3-(3-(4-bromophenyl) propoxy) propanoic acid (18.00 g, crude) as a yellow oil. ESI-MS: m/z=309.0 [M+23]⁺, 311.0 [M+3+23]⁺
Step 3: To a stirred solution of 3-(3-(4-bromophenyl) propoxy) propanoic acid (18.00 g, 0.06 mol) in t-BuOH (200 mL) was added DPPA (25.88 g, 0.09 mol) and TEA (25.38 g, 0.25 mol). The reaction mixture was stirred at 25° C. for 1 hour. The reaction mixture was stirred at 80° C. for 15 hours. The reaction mixture was slowly poured into ice water (200 mL) and extracted with EtOAc (200 mL×3). The combined organic layers were washed with brine, dried over Na₂SO₄ and concentrated to afford a crude product. The crude product was purified by silica gel chromatography (PE/EtOAc=100/1˜3/1) to give tert-butyl (2-(3-(4-bromophenyl) propoxy) ethyl) carbamate (6.00 g, 9.06% yield) as a yellow oil. ESI-MS: m/z=258.0 [M−100+1]⁺, 260.0 [M−100+3]⁺
Step 4: To a stirred solution of tert-butyl (2-(3-(4-bromophenyl) propoxy) ethyl) carbamate (6.00 g, 16.7 mmol) in THF (60 mL) under nitrogen was added NaH (0.60 g, 25.0 mmol) at 0° C. The mixture was stirred at 25° C. for 0.5 hours. Then CH₃I (2.37 g, 16.7 mmol) was slowly added dropwise. The reaction was stirred 25° C. for 16 hours. The reaction mixture was slowly poured into ice water (100 mL) and extracted with EtOAc (100 mL×3). The combined organic layers were washed with brine, dried over Na₂SO₄ and concentrated to afford a crude product. The crude product was purified by silica gel chromatography (PE/EtOAc=100/1˜3/1) to give tert-butyl (2-(3-(4-bromophenyl) propoxy) ethyl) (methyl)carbamate (5.00 g, 72.69% yield) as a yellow oil. ESI-MS: m/z=272.1 [M−100+1]⁺, 274.1 [M−100+3]⁺. H NMR (400 MHz, DMSO_d₆) δ 1.38 (s, 9H), 1.70-1.81 (m, 2H), 2.58 (t, J=7.6 Hz, 2H), 2.81 (d, J=7.2 Hz, 3H), 3.30 (t, J=5.6 Hz, 2H), 3.35 (t, J=6.4 Hz, 2H), 3.43 (t, J=5.6 Hz, 2H), 7.15 (d, J=8.4 Hz, 2H), 7.40-7.48 (m, 2H).

### Method 59

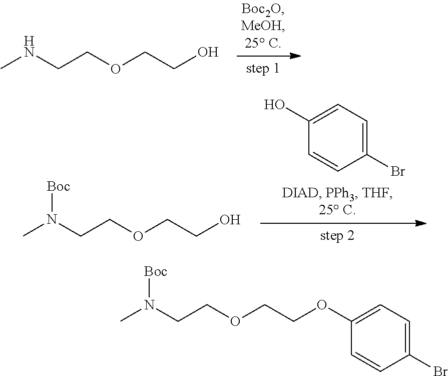

Step 1: To a solution of 2-[2-(methylamino) ethoxy]ethanol (4.50 g, 0.04 mol) in MeOH (50 mL) was added Boc₂O (12.37 g, 0.06 mol) at 25° C. The mixture was stirred at 25° C. for 4 hours. The reaction mixture was poured into water (100 mL) and extracted with DCM (30 mL×3). The combined organic layers were washed with brine, dried over Na₂SO₄ and concentrated to afford a crude product. The crude product was purified by silica gel chromatography (PE/EtOAc=100/1˜5/1) to give tert-butyl (2-(2-hydroxyethoxy) ethyl) (methyl)carbamate (5.00 g, 60.38% yield) as a yellow oil. ¹H NMR (400 MHz, CDCl₃) δ 1.45 (s, 9H), 2.90 (s, 3H), 3.40 (s, 2H), 3.58 (s, 4H), 3.70 (s, 2H).
Step 2: To a solution of tert-butyl (2-(2-hydroxyethoxy) ethyl) (methyl)carbamate (3.90 g, 18.0 mmol), PPh₃ (6.96 g, 27.0 mmol) and 4-bromophenol (4.59 g, 27.0 mmol) in THF (50 mL) was added DIAD (5.37 g, 27.0 mmol) at 0° C. The mixture was stirred at 25° C. for 4 hours. The reaction mixture was poured into water (100 mL) and extracted with DCM (30 mL×3). The combined organic layers were washed with brine, dried over Na₂SO₄ and concentrated to afford a crude product. The crude product was purified by silica gel chromatography (PE/EtOAc=100/1˜3/1) to give tert-butyl (2-(2-(4-bromophenoxy) ethoxy) ethyl) (methyl)carbamate (2.00 g, 30.05% yield) as a yellow solid. ESI-MS: m/z=273.9/275.9 [M−100+1]⁺. ¹H NMR (400 MHz, CDCl₃) δ 1.45 (s, 9H), 2.91 (s, 3H), 3.41 (t, J=5.6 Hz, 2H), 3.65 (t, J=5.6 Hz, 2H), 3.79 (t, J=5.6 Hz, 2H), 4.07 (t, J=5.6 Hz, 2H), 6.80 (d, J=9.2 Hz, 2H), 7.36 (d, J=9.2 Hz, 2H).

### General Scheme 14: Intermediates of Fluorinated Alkylamine

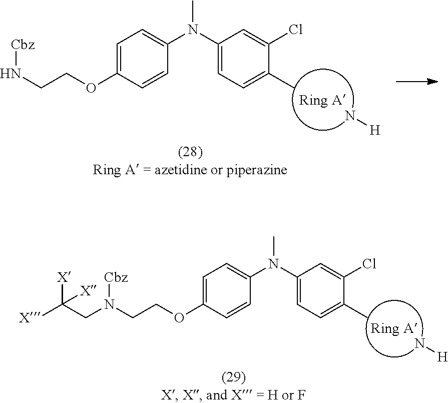

### Method 60

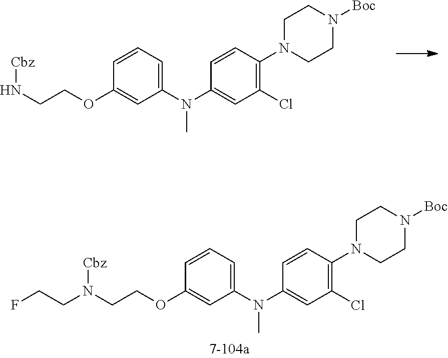 [SMILES: CN(c1cccc(OCCNC(=O)OCc2ccccc2)c1)c1ccc(N2CCN(C(=O)OC(C)(C)C)CC2)c(Cl)c1 ; CN(c1cccc(OCCN(CCF)C(=O)OCc2ccccc2)c1)c1ccc(N2CCN(C(=O)OC(C)(C)C)CC2)c(Cl)c1]

To a solution of tert-butyl 4-[4-[3-[2-(benzyloxycarbonylamino)ethoxy]-N-methyl-anilino]-2-chloro-phenyl]piperazine-1-carboxylate (255 mg, 0.43 mmol) in dry DMF (2.0 mL) cooled to 0° C. was added sodium hydride (50 mg, 1.25 mmol, 60% purity). The mixture was stirred for 30 minutes before the addition of 1-fluoro-2-iodo-ethane (242 mg, 1.39 mmol). The mixture was then heated to 40° C. with stirring for 2 hours. The mixture was cooled to room temperature, poured over H₂O, and extracted with EtOAc. The combined organic layers were washed with H₂O (3 times), washed with brine, dried over Na₂SO₄, filtered, and concentrated. The residue was purified by FCC on SiO₂ using a gradient of 10-50% EtOAc in hexanes. 7-104a was collected as an off-white solid with 9900 purity in 4600 yield (127 mg, 0.20 mmol). TLC (300% EtOAc/heptanes): R_{f}=0.29. LC/MS found 641.3 [M+H]⁺.

The following compounds 7-1 through 7-117 (27) shown in Table 7 were prepared by methods similar to those described for the preparation of 7-1, using the appropriate Boc-amine (24) and Glutarimide (21).

TABLE 7

| Compd | 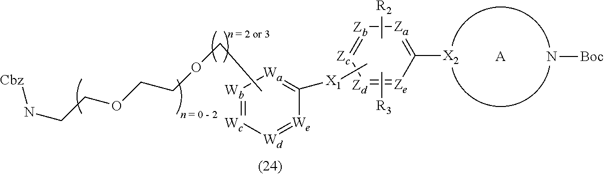 | | | |
|---|---|---|---|---|
| 7-1 | 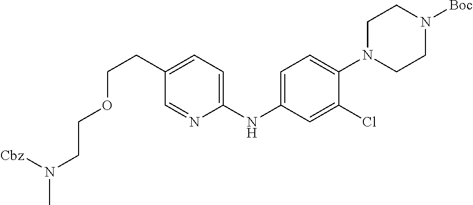 [SMILES: CN(CCOCCc1ccc(Nc2ccc(N3CCN(C(=O)OC(C)(C)C)CC3)c(Cl)c2)nc1)C(=O)OCc1ccccc1] | | | |
| 7-3 | 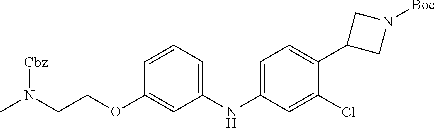 [SMILES: CN(CCOc1cccc(Nc2ccc(C3CN(C(=O)OC(C)(C)C)C3)c(Cl)c2)c1)C(=O)OCc1ccccc1] | | | |
| 7-4 | 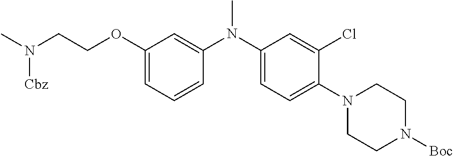 [SMILES: CN(CCOc1cccc(N(C)c2ccc(N3CCN(C(=O)OC(C)(C)C)CC3)c(Cl)c2)c1)C(=O)OCc1ccccc1] | | | |
| 7-5 | 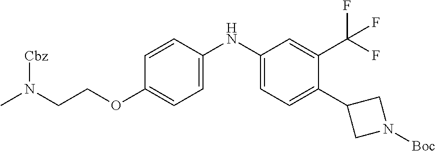 [SMILES: CN(CCOc1ccc(Nc2ccc(C3CN(C(=O)OC(C)(C)C)C3)c(C(F)(F)F)c2)cc1)C(=O)OCc1ccccc1] | | | |
| 7-6 | 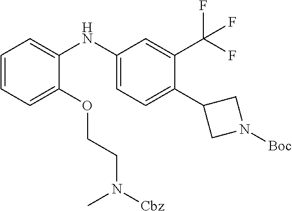 [SMILES: CN(CCOc1ccccc1Nc1ccc(C2CN(C(=O)OC(C)(C)C)C2)c(C(F)(F)F)c1)C(=O)OCc1ccccc1] | | | |
| 7-7 | 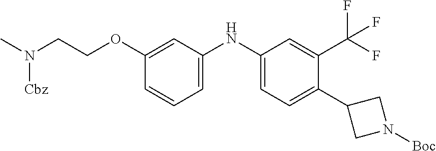 [SMILES: CN(CCOc1cccc(Nc2ccc(C3CN(C(=O)OC(C)(C)C)C3)c(C(F)(F)F)c2)c1)C(=O)OCc1ccccc1] | | | |
| 7-8 | 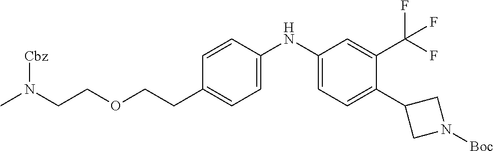 [SMILES: CN(CCOCCc1ccc(Nc2ccc(C3CN(C(=O)OC(C)(C)C)C3)c(C(F)(F)F)c2)cc1)C(=O)OCc1ccccc1] | | | |
| 7-9 | 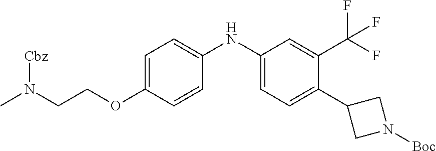 [SMILES: CN(CCOc1ccc(Nc2ccc(C3CN(C(=O)OC(C)(C)C)C3)c(C(F)(F)F)c2)cc1)C(=O)OCc1ccccc1] | | | |
| 7-10 | 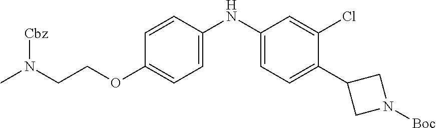 [SMILES: CN(CCOc1ccc(Nc2ccc(C3CN(C(=O)OC(C)(C)C)C3)c(Cl)c2)cc1)C(=O)OCc1ccccc1] | | | |
| 7-11 | 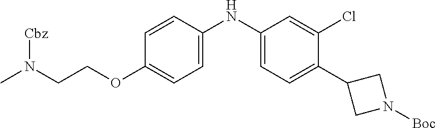 [SMILES: CN(CCOc1ccc(Nc2ccc(C3CN(C(=O)OC(C)(C)C)C3)c(Cl)c2)cc1)C(=O)OCc1ccccc1] | | | |
| 7-12 | 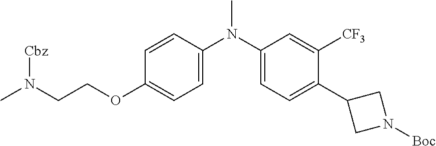 [SMILES: CN(CCOc1ccc(N(C)c2ccc(C3CN(C(=O)OC(C)(C)C)C3)c(C(F)(F)F)c2)cc1)C(=O)OCc1ccccc1] | | | |
| 7-13 | 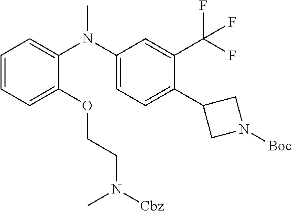 [SMILES: CN(CCOc1ccccc1N(C)c1ccc(C2CN(C(=O)OC(C)(C)C)C2)c(C(F)(F)F)c1)C(=O)OCc1ccccc1] | | | |
| 7-14 | 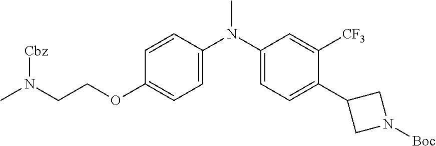 [SMILES: CN(CCOc1ccc(N(C)c2ccc(C3CN(C(=O)OC(C)(C)C)C3)c(C(F)(F)F)c2)cc1)C(=O)OCc1ccccc1] | | | |
| 7-15 | 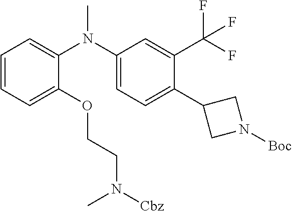 [SMILES: CN(CCOc1ccccc1N(C)c1ccc(C2CN(C(=O)OC(C)(C)C)C2)c(C(F)(F)F)c1)C(=O)OCc1ccccc1] | | | |
| 7-16 | 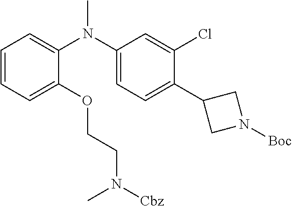 [SMILES: CN(CCOc1ccccc1N(C)c1ccc(C2CN(C(=O)OC(C)(C)C)C2)c(Cl)c1)C(=O)OCc1ccccc1] | | | |
| 7-17 | 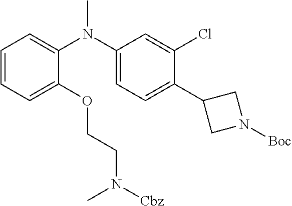 [SMILES: CN(CCOc1ccccc1N(C)c1ccc(C2CN(C(=O)OC(C)(C)C)C2)c(Cl)c1)C(=O)OCc1ccccc1] | | | |
| 7-18 | 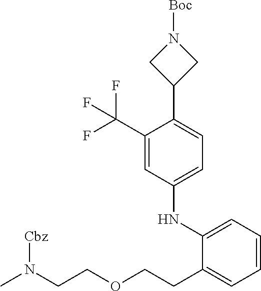 [SMILES: CN(CCOCCc1ccccc1Nc1ccc(C2CN(C(=O)OC(C)(C)C)C2)c(C(F)(F)F)c1)C(=O)OCc1ccccc1] | | | |
| 7-19 | 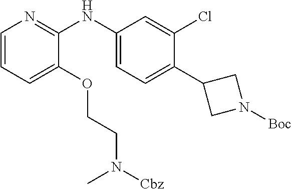 [SMILES: CN(CCOc1cccnc1Nc1ccc(C2CN(C(=O)OC(C)(C)C)C2)c(Cl)c1)C(=O)OCc1ccccc1] | | | |
| 7-20 | 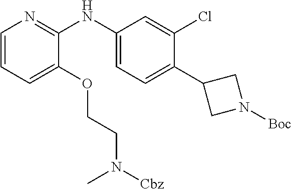 [SMILES: CN(CCOc1cccnc1Nc1ccc(C2CN(C(=O)OC(C)(C)C)C2)c(Cl)c1)C(=O)OCc1ccccc1] | | | |
| 7-21 | 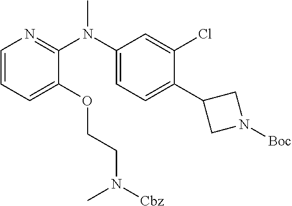 [SMILES: CN(CCOc1cccnc1N(C)c1ccc(C2CN(C(=O)OC(C)(C)C)C2)c(Cl)c1)C(=O)OCc1ccccc1] | | | |
| 7-22 | 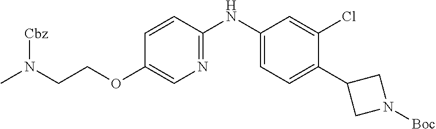 [SMILES: CN(CCOc1ccc(Nc2ccc(C3CN(C(=O)OC(C)(C)C)C3)c(Cl)c2)nc1)C(=O)OCc1ccccc1] | | | |
| 7-23 | 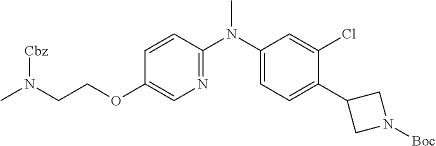 [SMILES: CN(CCOc1ccc(N(C)c2ccc(C3CN(C(=O)OC(C)(C)C)C3)c(Cl)c2)nc1)C(=O)OCc1ccccc1] | | | |
| 7-24 | 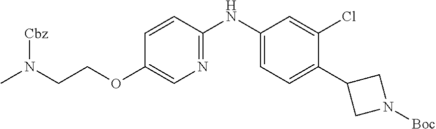 [SMILES: CN(CCOc1ccc(Nc2ccc(C3CN(C(=O)OC(C)(C)C)C3)c(Cl)c2)nc1)C(=O)OCc1ccccc1] | | | |
| 7-25 | 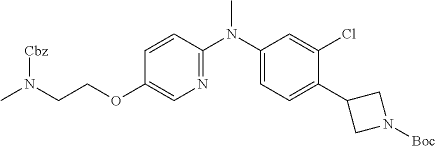 [SMILES: CN(CCOc1ccc(N(C)c2ccc(C3CN(C(=O)OC(C)(C)C)C3)c(Cl)c2)nc1)C(=O)OCc1ccccc1] | | | |
| 7-26 | 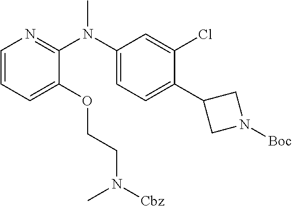 [SMILES: CN(CCOc1cccnc1N(C)c1ccc(C2CN(C(=O)OC(C)(C)C)C2)c(Cl)c1)C(=O)OCc1ccccc1] | | | |
| 7-27 | 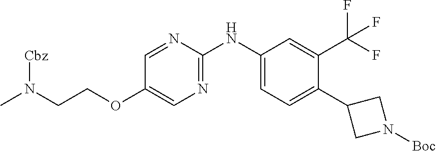 [SMILES: CN(CCOc1cnc(Nc2ccc(C3CN(C(=O)OC(C)(C)C)C3)c(C(F)(F)F)c2)nc1)C(=O)OCc1ccccc1] | | | |
| 7-28 | 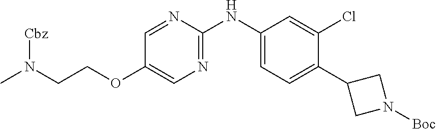 [SMILES: CN(CCOc1cnc(Nc2ccc(C3CN(C(=O)OC(C)(C)C)C3)c(Cl)c2)nc1)C(=O)OCc1ccccc1] | | | |
| 7-29 | 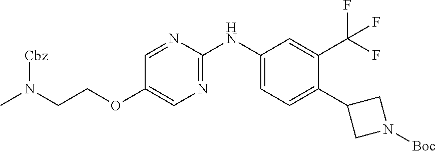 [SMILES: CN(CCOc1cnc(Nc2ccc(C3CN(C(=O)OC(C)(C)C)C3)c(C(F)(F)F)c2)nc1)C(=O)OCc1ccccc1] | | | |
| 7-30 | 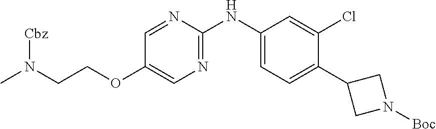 [SMILES: CN(CCOc1cnc(Nc2ccc(C3CN(C(=O)OC(C)(C)C)C3)c(Cl)c2)nc1)C(=O)OCc1ccccc1] | | | |
| 7-31 | 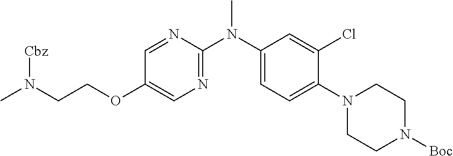 [SMILES: CN(CCOc1cnc(N(C)c2ccc(N3CCN(C(=O)OC(C)(C)C)CC3)c(Cl)c2)nc1)C(=O)OCc1ccccc1] | | | |
| 7-32 | 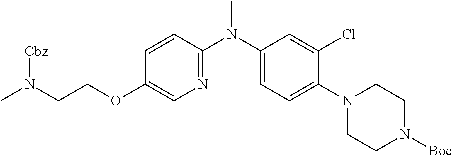 [SMILES: CN(CCOc1ccc(N(C)c2ccc(N3CCN(C(=O)OC(C)(C)C)CC3)c(Cl)c2)nc1)C(=O)OCc1ccccc1] | | | |
| 7-33 | 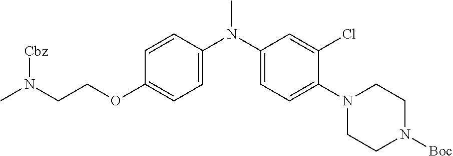 [SMILES: CN(CCOc1ccc(N(C)c2ccc(N3CCN(C(=O)OC(C)(C)C)CC3)c(Cl)c2)cc1)C(=O)OCc1ccccc1] | | | |
| 7-34 | 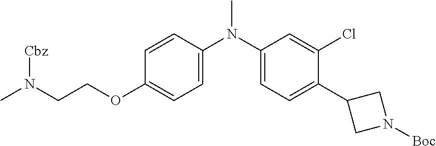 [SMILES: CN(CCOc1ccc(N(C)c2ccc(C3CN(C(=O)OC(C)(C)C)C3)c(Cl)c2)cc1)C(=O)OCc1ccccc1] | | | |
| 7-35 | 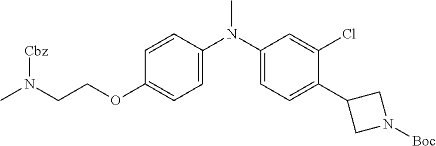 [SMILES: CN(CCOc1ccc(N(C)c2ccc(C3CN(C(=O)OC(C)(C)C)C3)c(Cl)c2)cc1)C(=O)OCc1ccccc1] | | | |
| 7-36 | 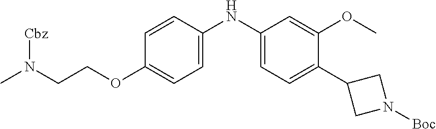 [SMILES: COc1cc(Nc2ccc(OCCN(C)C(=O)OCc3ccccc3)cc2)ccc1C1CN(C(=O)OC(C)(C)C)C1] | | | |
| 7-37 | 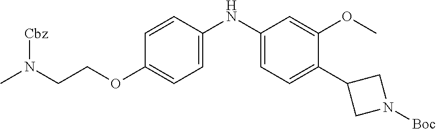 [SMILES: COc1cc(Nc2ccc(OCCN(C)C(=O)OCc3ccccc3)cc2)ccc1C1CN(C(=O)OC(C)(C)C)C1] | | | |
| 7-38 | 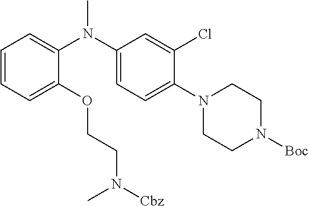 [SMILES: CN(CCOc1ccccc1N(C)c1ccc(N2CCN(C(=O)OC(C)(C)C)CC2)c(Cl)c1)C(=O)OCc1ccccc1] | | | |
| 7-39 | 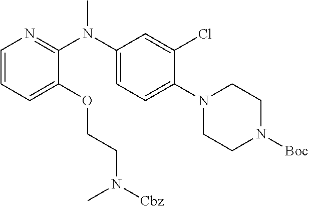 [SMILES: CN(CCOc1cccnc1N(C)c1ccc(N2CCN(C(=O)OC(C)(C)C)CC2)c(Cl)c1)C(=O)OCc1ccccc1] | | | |
| 7-40 | 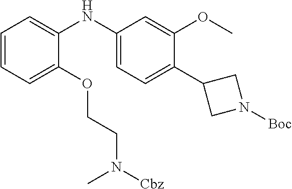 [SMILES: COc1cc(Nc2ccccc2OCCN(C)C(=O)OCc2ccccc2)ccc1C1CN(C(=O)OC(C)(C)C)C1] | | | |
| 7-41 | 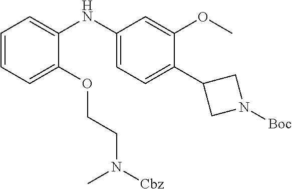 [SMILES: COc1cc(Nc2ccccc2OCCN(C)C(=O)OCc2ccccc2)ccc1C1CN(C(=O)OC(C)(C)C)C1] | | | |
| 7-42 | 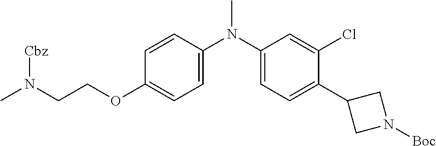 [SMILES: CN(CCOc1ccc(N(C)c2ccc(C3CN(C(=O)OC(C)(C)C)C3)c(Cl)c2)cc1)C(=O)OCc1ccccc1] | | | |
| 7-43 | 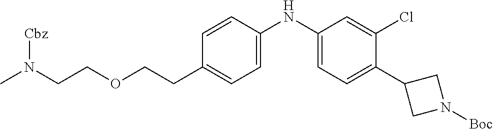 [SMILES: CN(CCOCCc1ccc(Nc2ccc(C3CN(C(=O)OC(C)(C)C)C3)c(Cl)c2)cc1)C(=O)OCc1ccccc1] | | | |
| 7-44 | 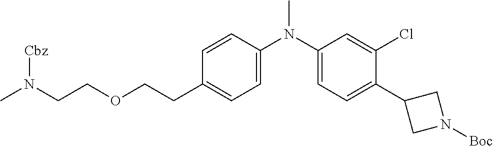 [SMILES: CN(CCOCCc1ccc(N(C)c2ccc(C3CN(C(=O)OC(C)(C)C)C3)c(Cl)c2)cc1)C(=O)OCc1ccccc1] | | | |
| 7-45 | 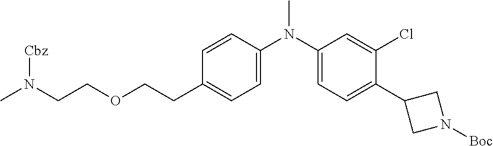 [SMILES: CN(CCOCCc1ccc(N(C)c2ccc(C3CN(C(=O)OC(C)(C)C)C3)c(Cl)c2)cc1)C(=O)OCc1ccccc1] | | | |
| 7-46 | 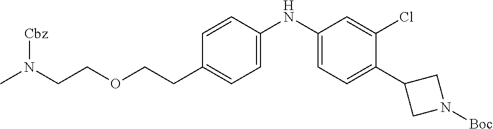 [SMILES: CN(CCOCCc1ccc(Nc2ccc(C3CN(C(=O)OC(C)(C)C)C3)c(Cl)c2)cc1)C(=O)OCc1ccccc1] | | | |
| 7-47 | 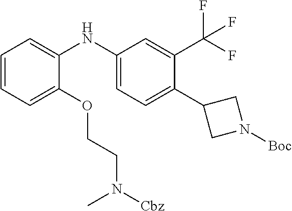 [SMILES: CN(CCOc1ccccc1Nc1ccc(C2CN(C(=O)OC(C)(C)C)C2)c(C(F)(F)F)c1)C(=O)OCc1ccccc1] | | | |
| 7-48 | 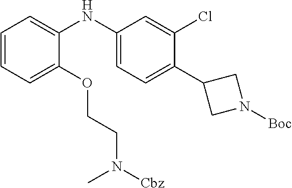 [SMILES: CN(CCOc1ccccc1Nc1ccc(C2CN(C(=O)OC(C)(C)C)C2)c(Cl)c1)C(=O)OCc1ccccc1] | | | |
| 7-49 | 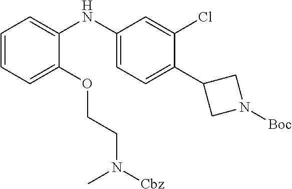 [SMILES: CN(CCOc1ccccc1Nc1ccc(C2CN(C(=O)OC(C)(C)C)C2)c(Cl)c1)C(=O)OCc1ccccc1] | | | |
| 7-50 | 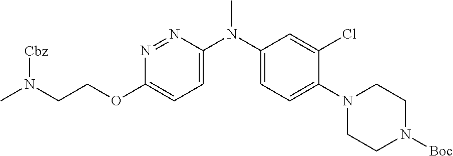 [SMILES: CN(CCOc1ccc(N(C)c2ccc(N3CCN(C(=O)OC(C)(C)C)CC3)c(Cl)c2)nn1)C(=O)OCc1ccccc1] | | | |
| 7-51 | 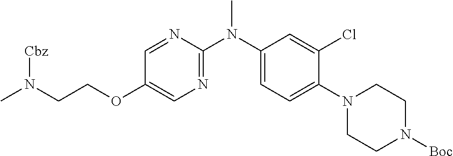 [SMILES: CN(CCOc1cnc(N(C)c2ccc(N3CCN(C(=O)OC(C)(C)C)CC3)c(Cl)c2)nc1)C(=O)OCc1ccccc1] | | | |
| 7-52 | 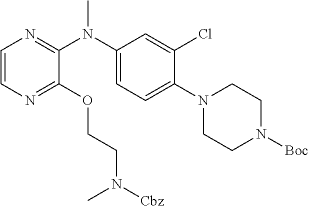 [SMILES: CN(CCOc1nccnc1N(C)c1ccc(N2CCN(C(=O)OC(C)(C)C)CC2)c(Cl)c1)C(=O)OCc1ccccc1] | | | |
| 7-53 | 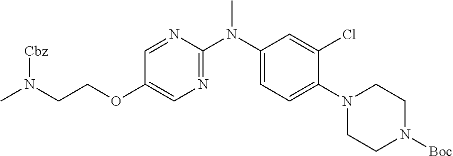 [SMILES: CN(CCOc1cnc(N(C)c2ccc(N3CCN(C(=O)OC(C)(C)C)CC3)c(Cl)c2)nc1)C(=O)OCc1ccccc1] | | | |
| 7-54 | 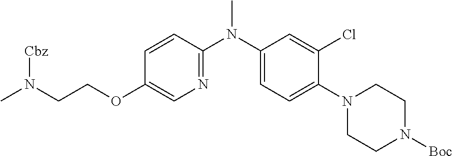 [SMILES: CN(CCOc1ccc(N(C)c2ccc(N3CCN(C(=O)OC(C)(C)C)CC3)c(Cl)c2)nc1)C(=O)OCc1ccccc1] | | | |
| 7-55 | 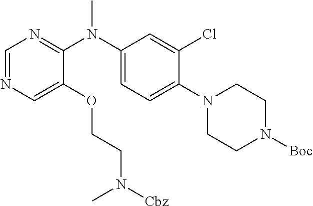 [SMILES: CN(CCOc1cncnc1N(C)c1ccc(N2CCN(C(=O)OC(C)(C)C)CC2)c(Cl)c1)C(=O)OCc1ccccc1] | | | |
| 7-56 | 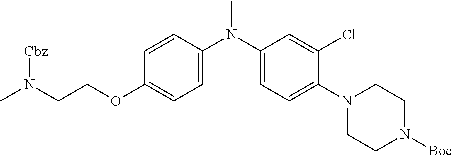 [SMILES: CN(CCOc1ccc(N(C)c2ccc(N3CCN(C(=O)OC(C)(C)C)CC3)c(Cl)c2)cc1)C(=O)OCc1ccccc1] | | | |
| 7-57 | 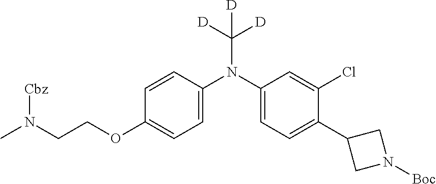 [SMILES: [2H]C([2H])([2H])N(c1ccc(OCCN(C)C(=O)OCc2ccccc2)cc1)c1ccc(C2CN(C(=O)OC(C)(C)C)C2)c(Cl)c1] | | | |
| 7-58 | 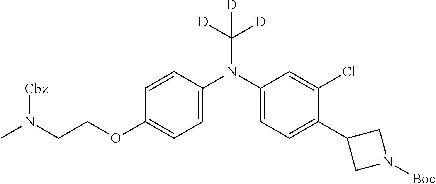 [SMILES: [2H]C([2H])([2H])N(c1ccc(OCCN(C)C(=O)OCc2ccccc2)cc1)c1ccc(C2CN(C(=O)OC(C)(C)C)C2)c(Cl)c1] | | | |
| 7-59 | 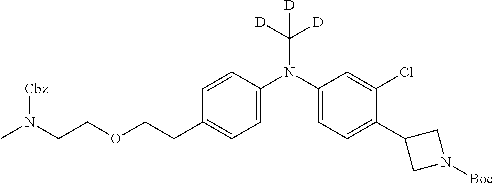 [SMILES: [2H]C([2H])([2H])N(c1ccc(CCOCCN(C)C(=O)OCc2ccccc2)cc1)c1ccc(C2CN(C(=O)OC(C)(C)C)C2)c(Cl)c1] | | | |
| 7-60 | 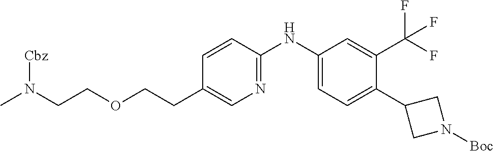 [SMILES: CN(CCOCCc1ccc(Nc2ccc(C3CN(C(=O)OC(C)(C)C)C3)c(C(F)(F)F)c2)nc1)C(=O)OCc1ccccc1] | | | |
| 7-61 | 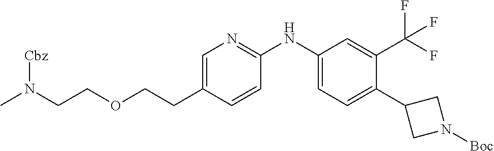 [SMILES: CN(CCOCCc1ccc(Nc2ccc(C3CN(C(=O)OC(C)(C)C)C3)c(C(F)(F)F)c2)nc1)C(=O)OCc1ccccc1] | | | |
| 7-62 | 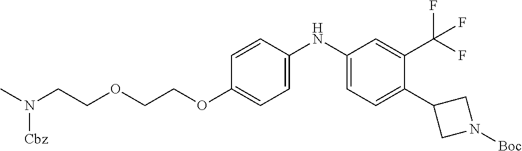 [SMILES: CN(CCOCCOc1ccc(Nc2ccc(C3CN(C(=O)OC(C)(C)C)C3)c(C(F)(F)F)c2)cc1)C(=O)OCc1ccccc1] | | | |
| 7-63 | 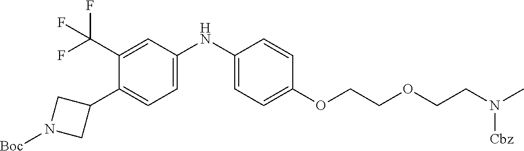 [SMILES: CN(CCOCCOc1ccc(Nc2ccc(C3CN(C(=O)OC(C)(C)C)C3)c(C(F)(F)F)c2)cc1)C(=O)OCc1ccccc1] | | | |
| 7-64 | 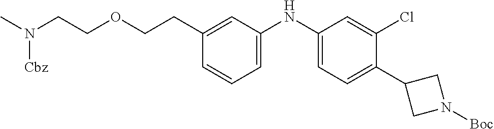 [SMILES: CN(CCOCCc1cccc(Nc2ccc(C3CN(C(=O)OC(C)(C)C)C3)c(Cl)c2)c1)C(=O)OCc1ccccc1] | | | |
| 7-65 | 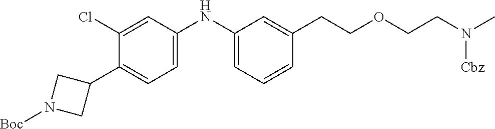 [SMILES: CN(CCOCCc1cccc(Nc2ccc(C3CN(C(=O)OC(C)(C)C)C3)c(Cl)c2)c1)C(=O)OCc1ccccc1] | | | |
| 7-66 | 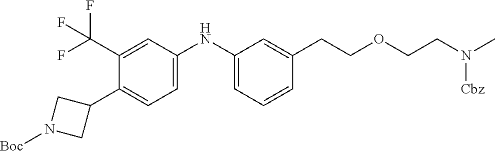 [SMILES: CN(CCOCCc1cccc(Nc2ccc(C3CN(C(=O)OC(C)(C)C)C3)c(C(F)(F)F)c2)c1)C(=O)OCc1ccccc1] | | | |
| 7-67 | 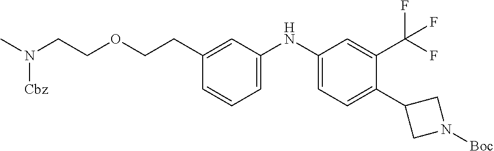 [SMILES: CN(CCOCCc1cccc(Nc2ccc(C3CN(C(=O)OC(C)(C)C)C3)c(C(F)(F)F)c2)c1)C(=O)OCc1ccccc1] | | | |
| 7-68 | 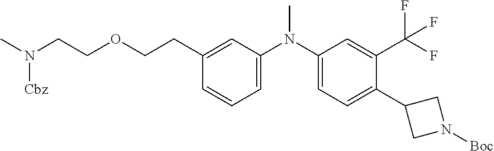 [SMILES: CN(CCOCCc1cccc(N(C)c2ccc(C3CN(C(=O)OC(C)(C)C)C3)c(C(F)(F)F)c2)c1)C(=O)OCc1ccccc1] | | | |
| 7-69 | 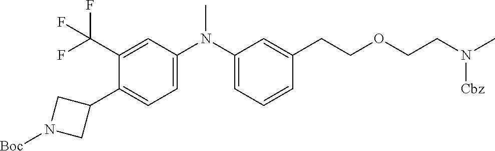 [SMILES: CN(CCOCCc1cccc(N(C)c2ccc(C3CN(C(=O)OC(C)(C)C)C3)c(C(F)(F)F)c2)c1)C(=O)OCc1ccccc1] | | | |
| 7-70 | 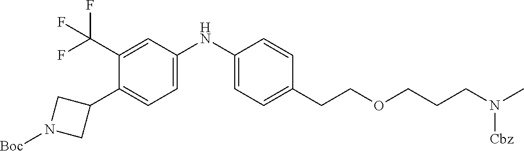 [SMILES: CN(CCCOCCc1ccc(Nc2ccc(C3CN(C(=O)OC(C)(C)C)C3)c(C(F)(F)F)c2)cc1)C(=O)OCc1ccccc1] | | | |
| 7-71 | 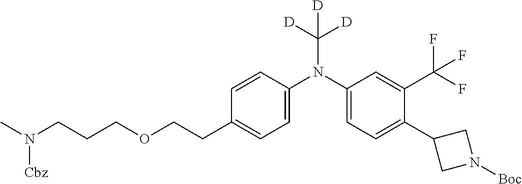 [SMILES: [2H]C([2H])([2H])N(c1ccc(CCOCCCN(C)C(=O)OCc2ccccc2)cc1)c1ccc(C2CN(C(=O)OC(C)(C)C)C2)c(C(F)(F)F)c1] | | | |
| 7-72 | 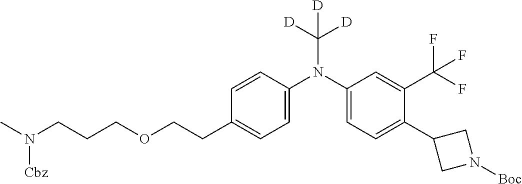 [SMILES: [2H]C([2H])([2H])N(c1ccc(CCOCCCN(C)C(=O)OCc2ccccc2)cc1)c1ccc(C2CN(C(=O)OC(C)(C)C)C2)c(C(F)(F)F)c1] | | | |
| 7-73 | 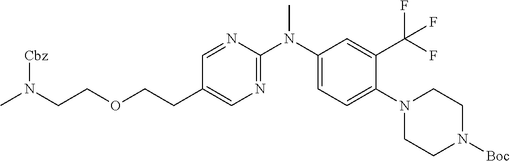 [SMILES: CN(CCOCCc1cnc(N(C)c2ccc(N3CCN(C(=O)OC(C)(C)C)CC3)c(C(F)(F)F)c2)nc1)C(=O)OCc1ccccc1] | | | |
| 7-74 | 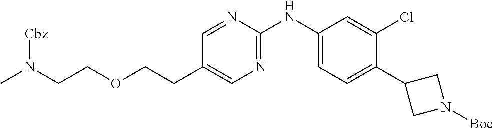 [SMILES: CN(CCOCCc1cnc(Nc2ccc(C3CN(C(=O)OC(C)(C)C)C3)c(Cl)c2)nc1)C(=O)OCc1ccccc1] | | | |
| 7-75 | 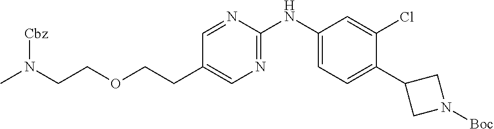 [SMILES: CN(CCOCCc1cnc(Nc2ccc(C3CN(C(=O)OC(C)(C)C)C3)c(Cl)c2)nc1)C(=O)OCc1ccccc1] | | | |
| 7-76 | 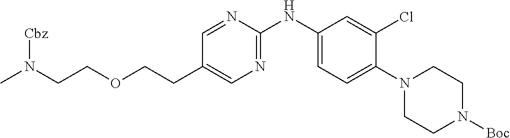 [SMILES: CN(CCOCCc1cnc(Nc2ccc(N3CCN(C(=O)OC(C)(C)C)CC3)c(Cl)c2)nc1)C(=O)OCc1ccccc1] | | | |
| 7-77 | 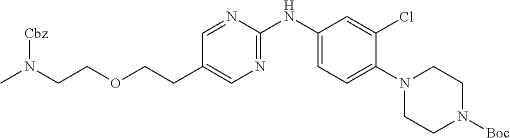 [SMILES: CN(CCOCCc1cnc(Nc2ccc(N3CCN(C(=O)OC(C)(C)C)CC3)c(Cl)c2)nc1)C(=O)OCc1ccccc1] | | | |
| 7-78 | 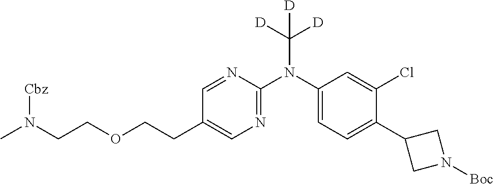 [SMILES: [2H]C([2H])([2H])N(c1ccc(C2CN(C(=O)OC(C)(C)C)C2)c(Cl)c1)c1ncc(CCOCCN(C)C(=O)OCc2ccccc2)cn1] | | | |
| 7-79 | 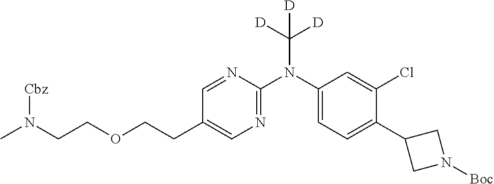 [SMILES: [2H]C([2H])([2H])N(c1ccc(C2CN(C(=O)OC(C)(C)C)C2)c(Cl)c1)c1ncc(CCOCCN(C)C(=O)OCc2ccccc2)cn1] | | | |
| 7-80 | 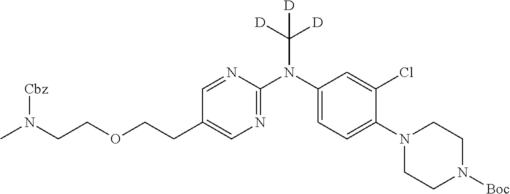 [SMILES: [2H]C([2H])([2H])N(c1ccc(N2CCN(C(=O)OC(C)(C)C)CC2)c(Cl)c1)c1ncc(CCOCCN(C)C(=O)OCc2ccccc2)cn1] | | | |
| 7-81 | 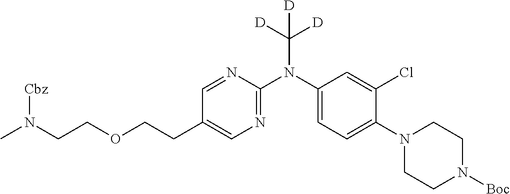 [SMILES: [2H]C([2H])([2H])N(c1ccc(N2CCN(C(=O)OC(C)(C)C)CC2)c(Cl)c1)c1ncc(CCOCCN(C)C(=O)OCc2ccccc2)cn1] | | | |
| 7-82 | 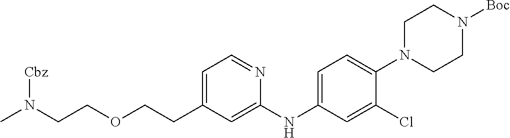 [SMILES: CN(CCOCCc1ccnc(Nc2ccc(N3CCN(C(=O)OC(C)(C)C)CC3)c(Cl)c2)c1)C(=O)OCc1ccccc1] | | | |
| 7-83 | 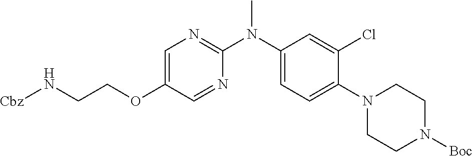 [SMILES: CN(c1ccc(N2CCN(C(=O)OC(C)(C)C)CC2)c(Cl)c1)c1ncc(OCCNC(=O)OCc2ccccc2)cn1] | | | |
| 7-84 | 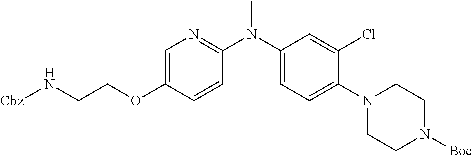 [SMILES: CN(c1ccc(N2CCN(C(=O)OC(C)(C)C)CC2)c(Cl)c1)c1ccc(OCCNC(=O)OCc2ccccc2)cn1] | | | |
| 7-85 | 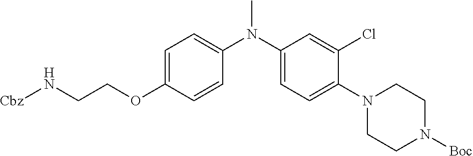 [SMILES: CN(c1ccc(OCCNC(=O)OCc2ccccc2)cc1)c1ccc(N2CCN(C(=O)OC(C)(C)C)CC2)c(Cl)c1] | | | |
| 7-86 | 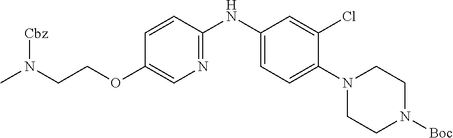 [SMILES: CN(CCOc1ccc(Nc2ccc(N3CCN(C(=O)OC(C)(C)C)CC3)c(Cl)c2)nc1)C(=O)OCc1ccccc1] | | | |
| 7-87 | 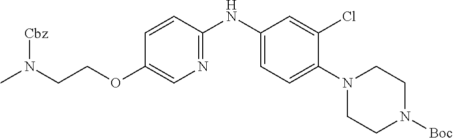 [SMILES: CN(CCOc1ccc(Nc2ccc(N3CCN(C(=O)OC(C)(C)C)CC3)c(Cl)c2)nc1)C(=O)OCc1ccccc1] | | | |
| 7-88 | 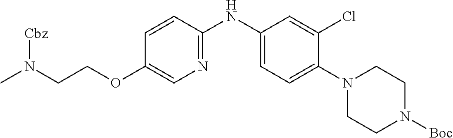 [SMILES: CN(CCOc1ccc(Nc2ccc(N3CCN(C(=O)OC(C)(C)C)CC3)c(Cl)c2)nc1)C(=O)OCc1ccccc1] | | | |
| 7-89 | 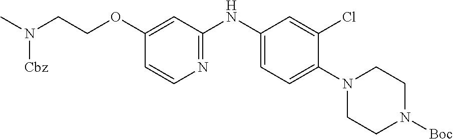 [SMILES: CN(CCOc1ccnc(Nc2ccc(N3CCN(C(=O)OC(C)(C)C)CC3)c(Cl)c2)c1)C(=O)OCc1ccccc1] | | | |
| 7-90 | 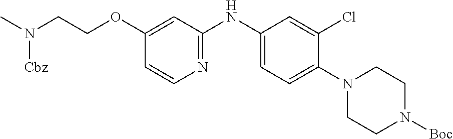 [SMILES: CN(CCOc1ccnc(Nc2ccc(N3CCN(C(=O)OC(C)(C)C)CC3)c(Cl)c2)c1)C(=O)OCc1ccccc1] | | | |
| 7-91 | 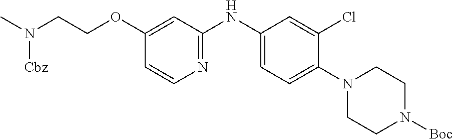 [SMILES: CN(CCOc1ccnc(Nc2ccc(N3CCN(C(=O)OC(C)(C)C)CC3)c(Cl)c2)c1)C(=O)OCc1ccccc1] | | | |
| 7-93 | 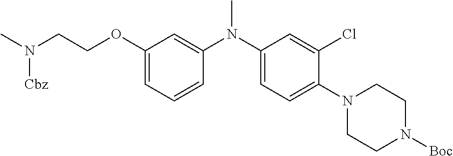 [SMILES: CN(CCOc1cccc(N(C)c2ccc(N3CCN(C(=O)OC(C)(C)C)CC3)c(Cl)c2)c1)C(=O)OCc1ccccc1] | | | |
| 7-94 | 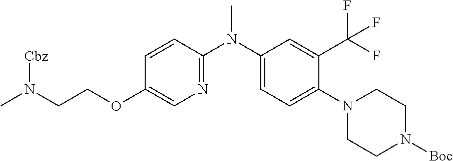 [SMILES: CN(CCOc1ccc(N(C)c2ccc(N3CCN(C(=O)OC(C)(C)C)CC3)c(C(F)(F)F)c2)nc1)C(=O)OCc1ccccc1] | | | |
| 7-95 | 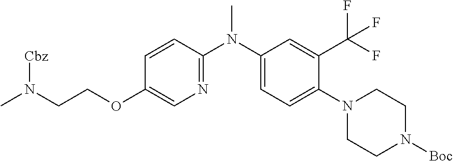 [SMILES: CN(CCOc1ccc(N(C)c2ccc(N3CCN(C(=O)OC(C)(C)C)CC3)c(C(F)(F)F)c2)nc1)C(=O)OCc1ccccc1] | | | |
| 7-96 | 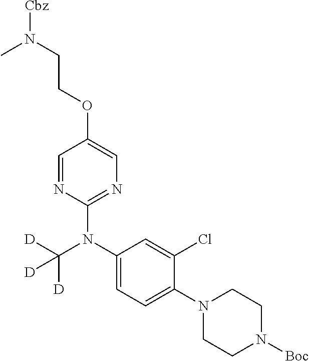 [SMILES: [2H]C([2H])([2H])N(c1ccc(N2CCN(C(=O)OC(C)(C)C)CC2)c(Cl)c1)c1ncc(OCCN(C)C(=O)OCc2ccccc2)cn1] | | | |
| 7-97 | 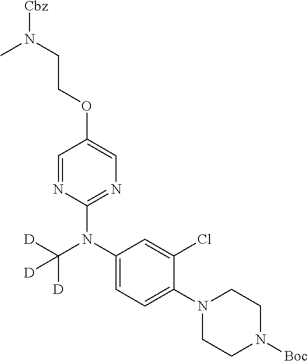 [SMILES: [2H]C([2H])([2H])N(c1ccc(N2CCN(C(=O)OC(C)(C)C)CC2)c(Cl)c1)c1ncc(OCCN(C)C(=O)OCc2ccccc2)cn1] | | | |
| 7-98 | 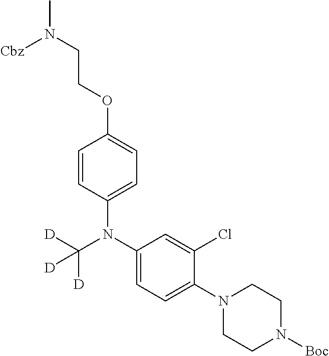 [SMILES: [2H]C([2H])([2H])N(c1ccc(OCCN(C)C(=O)OCc2ccccc2)cc1)c1ccc(N2CCN(C(=O)OC(C)(C)C)CC2)c(Cl)c1] | | | |
| 7-99 | 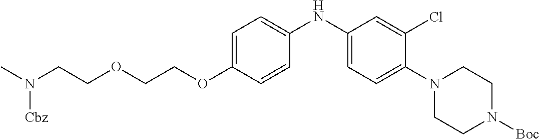 [SMILES: CN(CCOCCOc1ccc(Nc2ccc(N3CCN(C(=O)OC(C)(C)C)CC3)c(Cl)c2)cc1)C(=O)OCc1ccccc1] | | | |
| 7-100 | 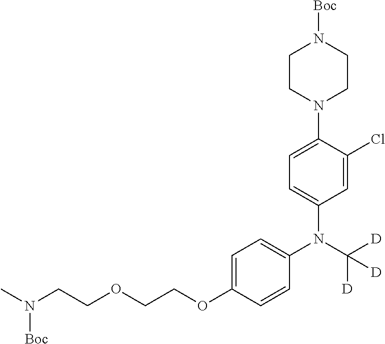 [SMILES: [2H]C([2H])([2H])N(c1ccc(OCCOCCN(C)C(=O)OC(C)(C)C)cc1)c1ccc(N2CCN(C(=O)OC(C)(C)C)CC2)c(Cl)c1] | | | |
| 7-101 | 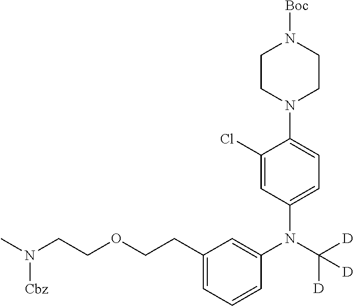 [SMILES: [2H]C([2H])([2H])N(c1cccc(CCOCCN(C)C(=O)OCc2ccccc2)c1)c1ccc(N2CCN(C(=O)OC(C)(C)C)CC2)c(Cl)c1] | | | |
| 7-102 | 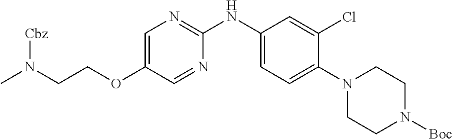 [SMILES: CN(CCOc1cnc(Nc2ccc(N3CCN(C(=O)OC(C)(C)C)CC3)c(Cl)c2)nc1)C(=O)OCc1ccccc1] | | | |
| 7-103 | 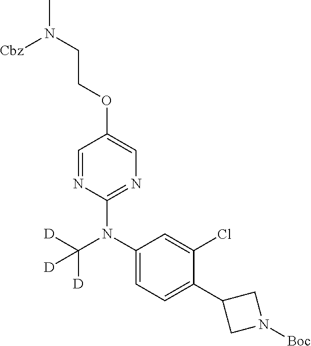 [SMILES: [2H]C([2H])([2H])N(c1ccc(C2CN(C(=O)OC(C)(C)C)C2)c(Cl)c1)c1ncc(OCCN(C)C(=O)OCc2ccccc2)cn1] | | | |
| 7-104 | 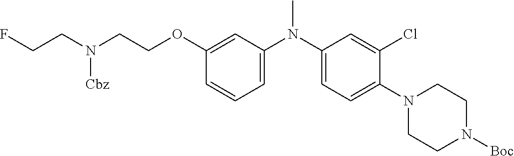 [SMILES: CN(c1cccc(OCCN(CCF)C(=O)OCc2ccccc2)c1)c1ccc(N2CCN(C(=O)OC(C)(C)C)CC2)c(Cl)c1] | | | |
| 7-105 | 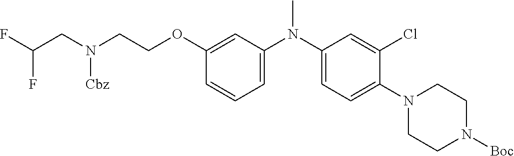 [SMILES: CN(c1cccc(OCCN(CC(F)F)C(=O)OCc2ccccc2)c1)c1ccc(N2CCN(C(=O)OC(C)(C)C)CC2)c(Cl)c1] | | | |
| 7-106 | 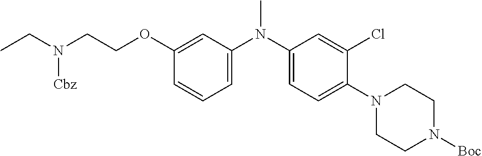 [SMILES: CCN(CCOc1cccc(N(C)c2ccc(N3CCN(C(=O)OC(C)(C)C)CC3)c(Cl)c2)c1)C(=O)OCc1ccccc1] | | | |
| 7-107 | 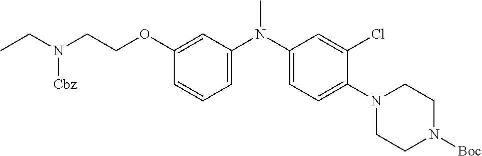 [SMILES: CCN(CCOc1cccc(N(C)c2ccc(N3CCN(C(=O)OC(C)(C)C)CC3)c(Cl)c2)c1)C(=O)OCc1ccccc1] | | | |
| 7-108 | 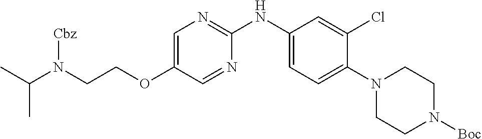 [SMILES: CC(C)N(CCOc1cnc(Nc2ccc(N3CCN(C(=O)OC(C)(C)C)CC3)c(Cl)c2)nc1)C(=O)OCc1ccccc1] | | | |
| 7-109 | 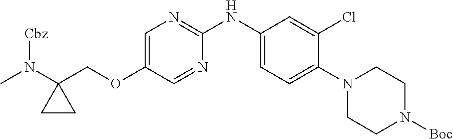 [SMILES: CN(C(=O)OCc1ccccc1)C1(COc2cnc(Nc3ccc(N4CCN(C(=O)OC(C)(C)C)CC4)c(Cl)c3)nc2)CC1] | | | |
| 7-110 | 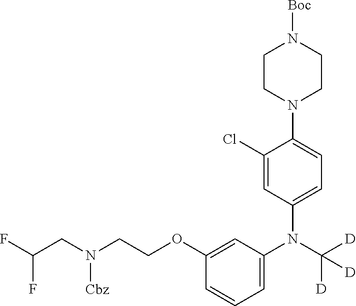 [SMILES: [2H]C([2H])([2H])N(c1cccc(OCCN(CC(F)F)C(=O)OCc2ccccc2)c1)c1ccc(N2CCN(C(=O)OC(C)(C)C)CC2)c(Cl)c1] | | | |
| 7-111 | 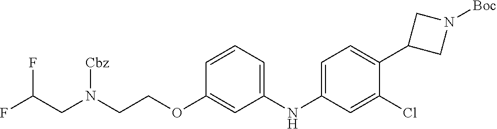 [SMILES: CC(C)(C)OC(=O)N1CC(c2ccc(Nc3cccc(OCCN(CC(F)F)C(=O)OCc4ccccc4)c3)cc2Cl)C1] | | | |
| 7-112 | 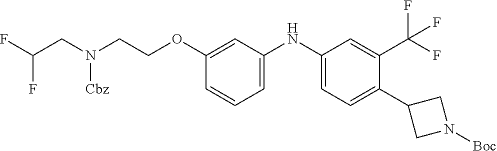 [SMILES: CC(C)(C)OC(=O)N1CC(c2ccc(Nc3cccc(OCCN(CC(F)F)C(=O)OCc4ccccc4)c3)cc2C(F)(F)F)C1] | | | |
| 7-113 | 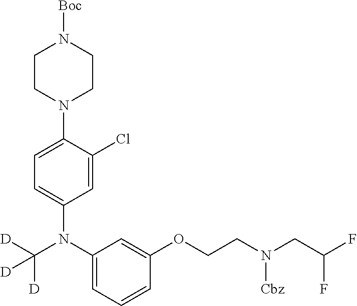 [SMILES: [2H]C([2H])([2H])N(c1cccc(OCCN(CC(F)F)C(=O)OCc2ccccc2)c1)c1ccc(N2CCN(C(=O)OC(C)(C)C)CC2)c(Cl)c1] | | | |
| 7-114 | 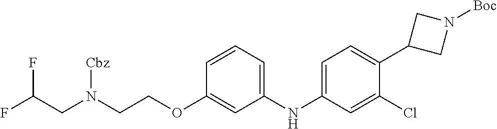 [SMILES: CC(C)(C)OC(=O)N1CC(c2ccc(Nc3cccc(OCCN(CC(F)F)C(=O)OCc4ccccc4)c3)cc2Cl)C1] | | | |
| 7-115 | 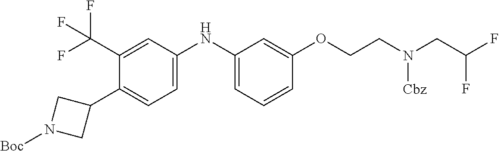 [SMILES: CC(C)(C)OC(=O)N1CC(c2ccc(Nc3cccc(OCCN(CC(F)F)C(=O)OCc4ccccc4)c3)cc2C(F)(F)F)C1] | | | |
| 7-116 | 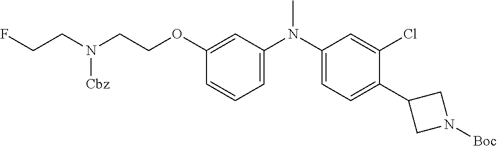 [SMILES: CN(c1cccc(OCCN(CCF)C(=O)OCc2ccccc2)c1)c1ccc(C2CN(C(=O)OC(C)(C)C)C2)c(Cl)c1] | | | |
| 7-117 | 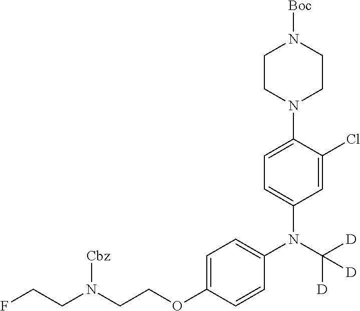 [SMILES: [2H]C([2H])([2H])N(c1ccc(OCCN(CCF)C(=O)OCc2ccccc2)cc1)c1ccc(N2CCN(C(=O)OC(C)(C)C)CC2)c(Cl)c1] | | | |
| | Compd | 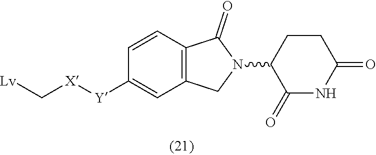 | | |
| | 7-1 | 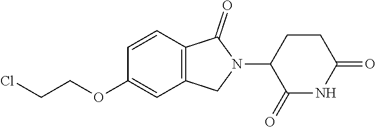 [SMILES: O=C1CCC(N2Cc3cc(OCCCl)ccc3C2=O)C(=O)N1] | | |
| | 7-3 | 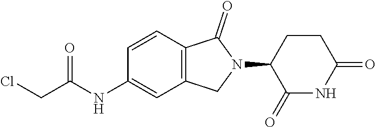 [SMILES: O=C1CC[C@H](N2Cc3cc(NC(=O)CCl)ccc3C2=O)C(=O)N1] | | |
| | 7-4 | 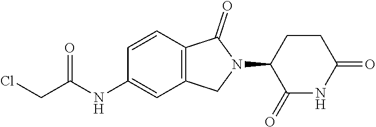 [SMILES: O=C1CC[C@H](N2Cc3cc(NC(=O)CCl)ccc3C2=O)C(=O)N1] | | |
| | 7-5 | 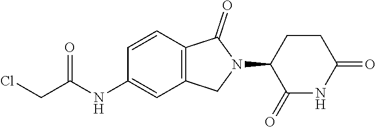 [SMILES: O=C1CC[C@H](N2Cc3cc(NC(=O)CCl)ccc3C2=O)C(=O)N1] | | |
| | 7-6 | 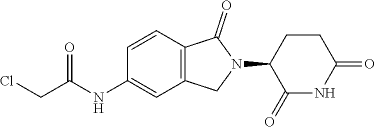 [SMILES: O=C1CC[C@H](N2Cc3cc(NC(=O)CCl)ccc3C2=O)C(=O)N1] | | |
| | 7-7 | 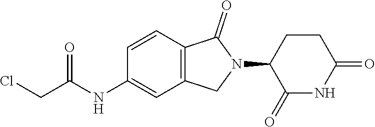 [SMILES: O=C1CC[C@H](N2Cc3cc(NC(=O)CCl)ccc3C2=O)C(=O)N1] | | |
| | 7-8 | 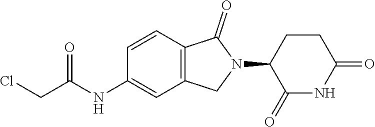 [SMILES: O=C1CC[C@H](N2Cc3cc(NC(=O)CCl)ccc3C2=O)C(=O)N1] | | |
| | 7-9 | 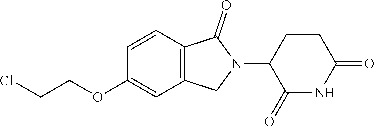 [SMILES: O=C1CCC(N2Cc3cc(OCCCl)ccc3C2=O)C(=O)N1] | | |
| | 7-10 | 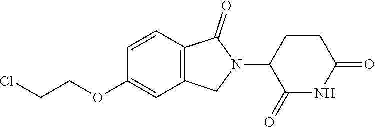 [SMILES: O=C1CCC(N2Cc3cc(OCCCl)ccc3C2=O)C(=O)N1] | | |
| | 7-11 | 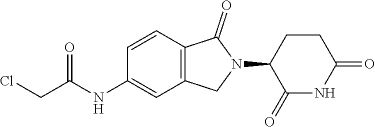 [SMILES: O=C1CC[C@H](N2Cc3cc(NC(=O)CCl)ccc3C2=O)C(=O)N1] | | |
| | 7-12 | 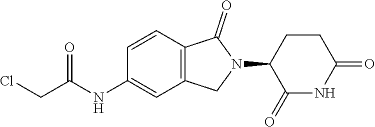 [SMILES: O=C1CC[C@H](N2Cc3cc(NC(=O)CCl)ccc3C2=O)C(=O)N1] | | |
| | 7-13 | 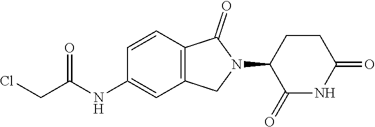 [SMILES: O=C1CC[C@H](N2Cc3cc(NC(=O)CCl)ccc3C2=O)C(=O)N1] | | |
| | 7-14 | 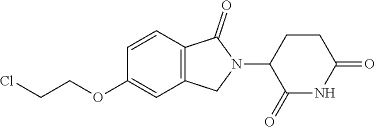 [SMILES: O=C1CCC(N2Cc3cc(OCCCl)ccc3C2=O)C(=O)N1] | | |
| | 7-15 | 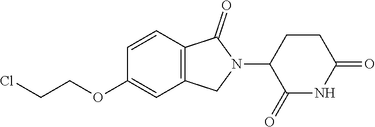 [SMILES: O=C1CCC(N2Cc3cc(OCCCl)ccc3C2=O)C(=O)N1] | | |
| | 7-16 | 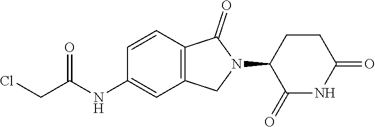 [SMILES: O=C1CC[C@H](N2Cc3cc(NC(=O)CCl)ccc3C2=O)C(=O)N1] | | |
| | 7-17 | 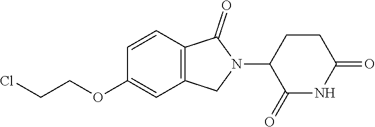 [SMILES: O=C1CCC(N2Cc3cc(OCCCl)ccc3C2=O)C(=O)N1] | | |
| | 7-18 | 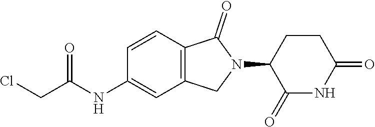 [SMILES: O=C1CC[C@H](N2Cc3cc(NC(=O)CCl)ccc3C2=O)C(=O)N1] | | |
| | 7-19 | 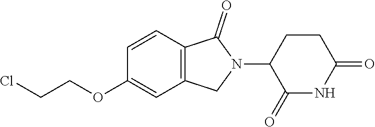 [SMILES: O=C1CCC(N2Cc3cc(OCCCl)ccc3C2=O)C(=O)N1] | | |
| | 7-20 | 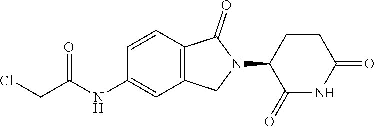 [SMILES: O=C1CC[C@H](N2Cc3cc(NC(=O)CCl)ccc3C2=O)C(=O)N1] | | |
| | 7-21 | 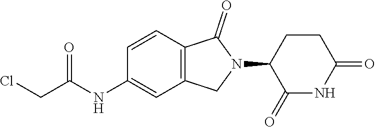 [SMILES: O=C1CC[C@H](N2Cc3cc(NC(=O)CCl)ccc3C2=O)C(=O)N1] | | |
| | 7-22 | 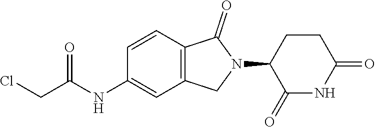 [SMILES: O=C1CC[C@H](N2Cc3cc(NC(=O)CCl)ccc3C2=O)C(=O)N1] | | |
| | 7-23 | 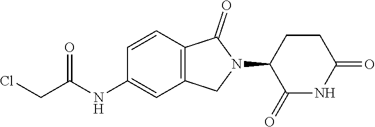 [SMILES: O=C1CC[C@H](N2Cc3cc(NC(=O)CCl)ccc3C2=O)C(=O)N1] | | |
| | 7-24 | 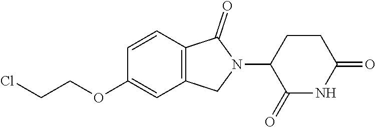 [SMILES: O=C1CCC(N2Cc3cc(OCCCl)ccc3C2=O)C(=O)N1] | | |
| | 7-25 | 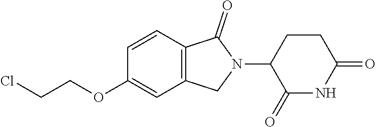 [SMILES: O=C1CCC(N2Cc3cc(OCCCl)ccc3C2=O)C(=O)N1] | | |
| | 7-26 | 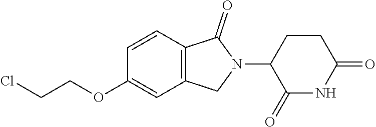 [SMILES: O=C1CCC(N2Cc3cc(OCCCl)ccc3C2=O)C(=O)N1] | | |
| | 7-27 | 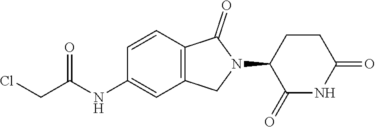 [SMILES: O=C1CC[C@H](N2Cc3cc(NC(=O)CCl)ccc3C2=O)C(=O)N1] | | |
| | 7-28 | 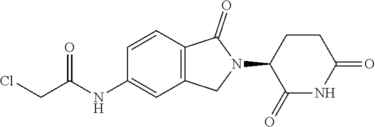 [SMILES: O=C1CC[C@H](N2Cc3cc(NC(=O)CCl)ccc3C2=O)C(=O)N1] | | |
| | 7-29 | 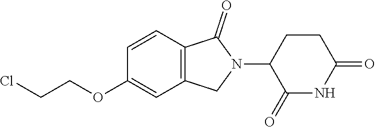 [SMILES: O=C1CCC(N2Cc3cc(OCCCl)ccc3C2=O)C(=O)N1] | | |
| | 7-30 | 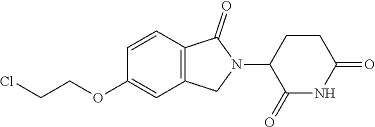 [SMILES: O=C1CCC(N2Cc3cc(OCCCl)ccc3C2=O)C(=O)N1] | | |
| | 7-31 | 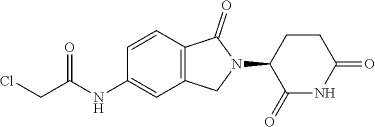 [SMILES: O=C1CC[C@H](N2Cc3cc(NC(=O)CCl)ccc3C2=O)C(=O)N1] | | |
| | 7-32 | 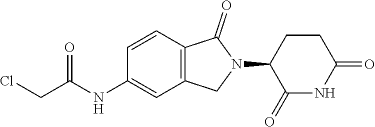 [SMILES: O=C1CC[C@H](N2Cc3cc(NC(=O)CCl)ccc3C2=O)C(=O)N1] | | |
| | 7-33 | 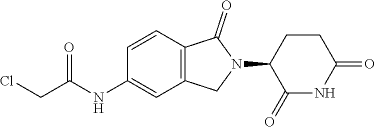 [SMILES: O=C1CC[C@H](N2Cc3cc(NC(=O)CCl)ccc3C2=O)C(=O)N1] | | |
| | 7-34 | 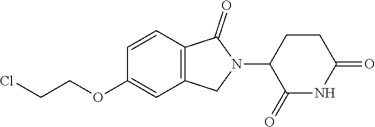 [SMILES: O=C1CCC(N2Cc3cc(OCCCl)ccc3C2=O)C(=O)N1] | | |
| | 7-35 | 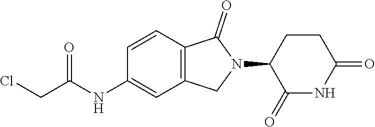 [SMILES: O=C1CC[C@H](N2Cc3cc(NC(=O)CCl)ccc3C2=O)C(=O)N1] | | |
| | 7-36 | 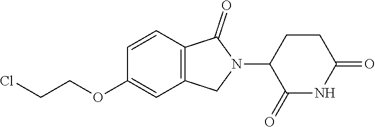 [SMILES: O=C1CCC(N2Cc3cc(OCCCl)ccc3C2=O)C(=O)N1] | | |
| | 7-37 | 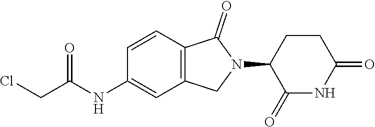 [SMILES: O=C1CC[C@H](N2Cc3cc(NC(=O)CCl)ccc3C2=O)C(=O)N1] | | |
| | 7-38 | 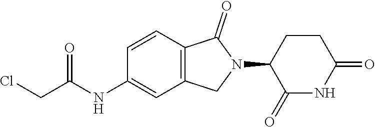 [SMILES: O=C1CC[C@H](N2Cc3cc(NC(=O)CCl)ccc3C2=O)C(=O)N1] | | |
| | 7-39 | 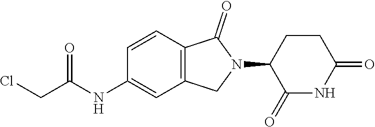 [SMILES: O=C1CC[C@H](N2Cc3cc(NC(=O)CCl)ccc3C2=O)C(=O)N1] | | |
| | 7-40 | 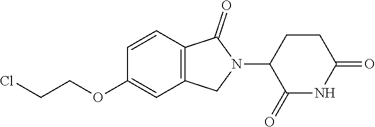 [SMILES: O=C1CCC(N2Cc3cc(OCCCl)ccc3C2=O)C(=O)N1] | | |
| | 7-41 | 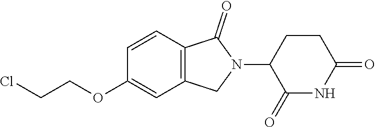 [SMILES: O=C1CCC(N2Cc3cc(OCCCl)ccc3C2=O)C(=O)N1] | | |
| | 7-42 | 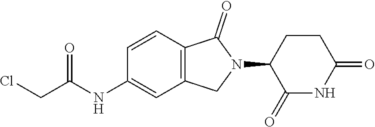 [SMILES: O=C1CC[C@H](N2Cc3cc(NC(=O)CCl)ccc3C2=O)C(=O)N1] | | |
| | 7-43 | 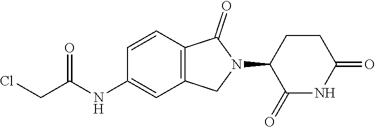 [SMILES: O=C1CC[C@H](N2Cc3cc(NC(=O)CCl)ccc3C2=O)C(=O)N1] | | |
| | 7-44 | 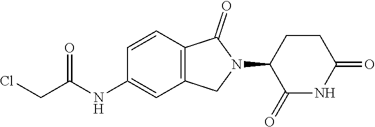 [SMILES: O=C1CC[C@H](N2Cc3cc(NC(=O)CCl)ccc3C2=O)C(=O)N1] | | |
| | 7-45 | 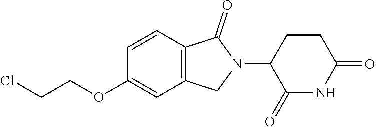 [SMILES: O=C1CCC(N2Cc3cc(OCCCl)ccc3C2=O)C(=O)N1] | | |
| | 7-46 | 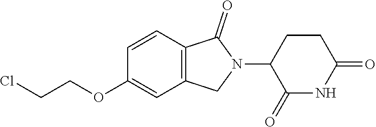 [SMILES: O=C1CCC(N2Cc3cc(OCCCl)ccc3C2=O)C(=O)N1] | | |
| | 7-47 | 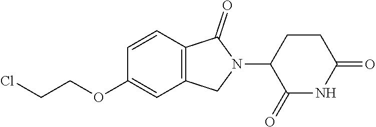 [SMILES: O=C1CCC(N2Cc3cc(OCCCl)ccc3C2=O)C(=O)N1] | | |
| | 7-48 | 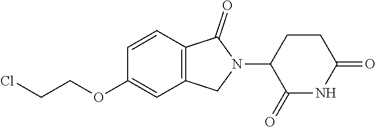 [SMILES: O=C1CCC(N2Cc3cc(OCCCl)ccc3C2=O)C(=O)N1] | | |
| | 7-49 | 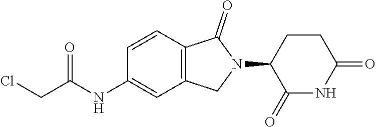 [SMILES: O=C1CC[C@H](N2Cc3cc(NC(=O)CCl)ccc3C2=O)C(=O)N1] | | |
| | 7-50 | 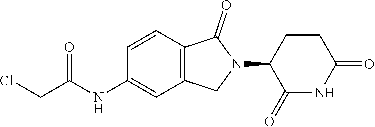 [SMILES: O=C1CC[C@H](N2Cc3cc(NC(=O)CCl)ccc3C2=O)C(=O)N1] | | |
| | 7-51 | 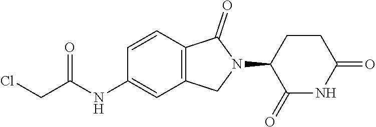 [SMILES: O=C1CC[C@H](N2Cc3cc(NC(=O)CCl)ccc3C2=O)C(=O)N1] | | |
| | 7-52 | 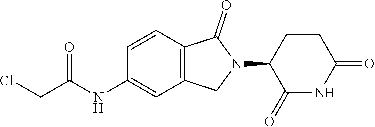 [SMILES: O=C1CC[C@H](N2Cc3cc(NC(=O)CCl)ccc3C2=O)C(=O)N1] | | |
| | 7-53 | 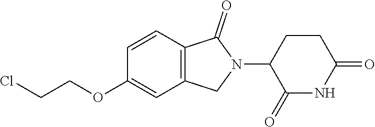 [SMILES: O=C1CCC(N2Cc3cc(OCCCl)ccc3C2=O)C(=O)N1] | | |
| | 7-54 | 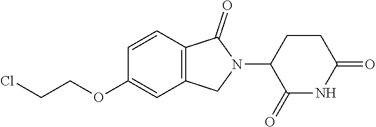 [SMILES: O=C1CCC(N2Cc3cc(OCCCl)ccc3C2=O)C(=O)N1] | | |
| | 7-55 | 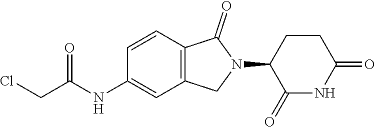 [SMILES: O=C1CC[C@H](N2Cc3cc(NC(=O)CCl)ccc3C2=O)C(=O)N1] | | |
| | 7-56 | 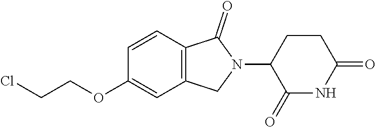 [SMILES: O=C1CCC(N2Cc3cc(OCCCl)ccc3C2=O)C(=O)N1] | | |
| | 7-57 | 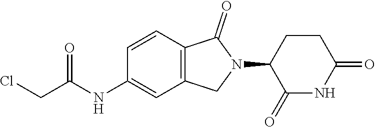 [SMILES: O=C1CC[C@H](N2Cc3cc(NC(=O)CCl)ccc3C2=O)C(=O)N1] | | |
| | 7-58 | 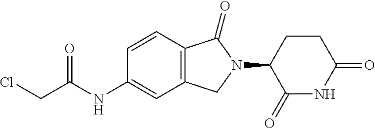 [SMILES: O=C1CC[C@H](N2Cc3cc(NC(=O)CCl)ccc3C2=O)C(=O)N1] | | |
| | 7-59 | 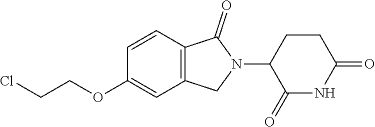 [SMILES: O=C1CCC(N2Cc3cc(OCCCl)ccc3C2=O)C(=O)N1] | | |
| | 7-60 | 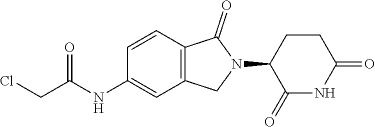 [SMILES: O=C1CC[C@H](N2Cc3cc(NC(=O)CCl)ccc3C2=O)C(=O)N1] | | |
| | 7-61 | 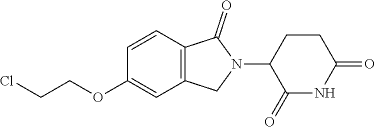 [SMILES: O=C1CCC(N2Cc3cc(OCCCl)ccc3C2=O)C(=O)N1] | | |
| | 7-62 | 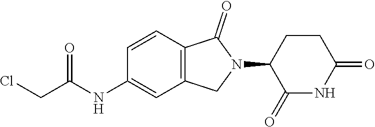 [SMILES: O=C1CC[C@H](N2Cc3cc(NC(=O)CCl)ccc3C2=O)C(=O)N1] | | |
| | 7-63 | 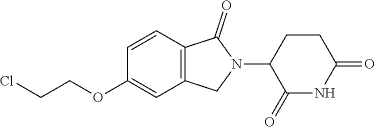 [SMILES: O=C1CCC(N2Cc3cc(OCCCl)ccc3C2=O)C(=O)N1] | | |
| | 7-64 | 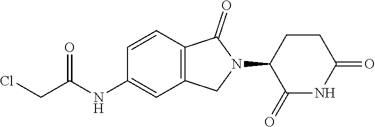 [SMILES: O=C1CC[C@H](N2Cc3cc(NC(=O)CCl)ccc3C2=O)C(=O)N1] | | |
| | 7-65 | 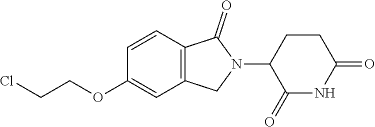 [SMILES: O=C1CCC(N2Cc3cc(OCCCl)ccc3C2=O)C(=O)N1] | | |
| | 7-66 | 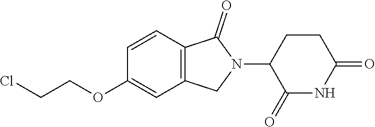 [SMILES: O=C1CCC(N2Cc3cc(OCCCl)ccc3C2=O)C(=O)N1] | | |
| | 7-67 | 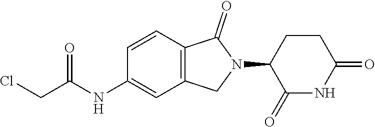 [SMILES: O=C1CC[C@H](N2Cc3cc(NC(=O)CCl)ccc3C2=O)C(=O)N1] | | |
| | 7-68 | 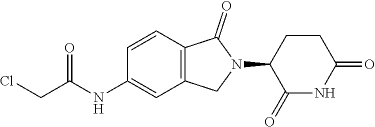 [SMILES: O=C1CC[C@H](N2Cc3cc(NC(=O)CCl)ccc3C2=O)C(=O)N1] | | |
| | 7-69 | 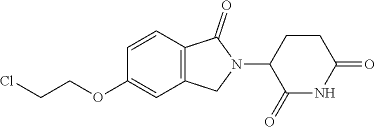 [SMILES: O=C1CCC(N2Cc3cc(OCCCl)ccc3C2=O)C(=O)N1] | | |
| | 7-70 | 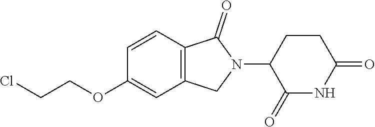 [SMILES: O=C1CCC(N2Cc3cc(OCCCl)ccc3C2=O)C(=O)N1] | | |
| | 7-71 | 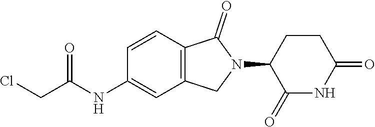 [SMILES: O=C1CC[C@H](N2Cc3cc(NC(=O)CCl)ccc3C2=O)C(=O)N1] | | |
| | 7-72 | 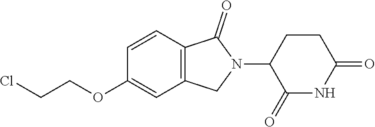 [SMILES: O=C1CCC(N2Cc3cc(OCCCl)ccc3C2=O)C(=O)N1] | | |
| | 7-73 | 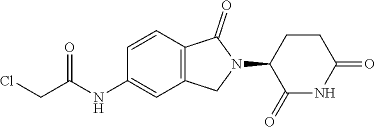 [SMILES: O=C1CC[C@H](N2Cc3cc(NC(=O)CCl)ccc3C2=O)C(=O)N1] | | |
| | 7-74 | 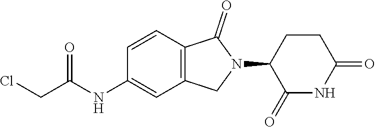 [SMILES: O=C1CC[C@H](N2Cc3cc(NC(=O)CCl)ccc3C2=O)C(=O)N1] | | |
| | 7-75 | 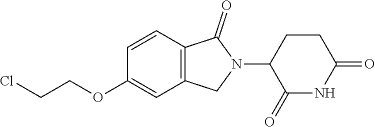 [SMILES: O=C1CCC(N2Cc3cc(OCCCl)ccc3C2=O)C(=O)N1] | | |
| | 7-76 | 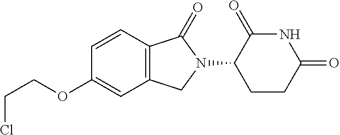 [SMILES: O=C1CC[C@H](N2Cc3cc(OCCCl)ccc3C2=O)C(=O)N1] | | |
| | 7-77 | 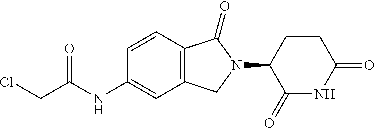 [SMILES: O=C1CC[C@H](N2Cc3cc(NC(=O)CCl)ccc3C2=O)C(=O)N1] | | |
| | 7-78 | 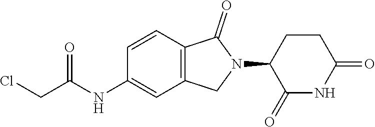 [SMILES: O=C1CC[C@H](N2Cc3cc(NC(=O)CCl)ccc3C2=O)C(=O)N1] | | |
| | 7-79 | 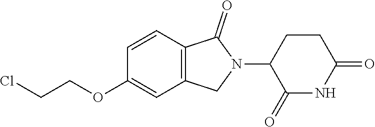 [SMILES: O=C1CCC(N2Cc3cc(OCCCl)ccc3C2=O)C(=O)N1] | | |
| | 7-80 | 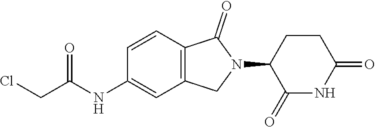 [SMILES: O=C1CC[C@H](N2Cc3cc(NC(=O)CCl)ccc3C2=O)C(=O)N1] | | |
| | 7-81 | 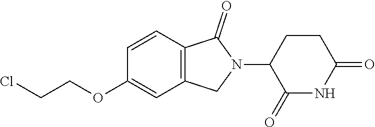 [SMILES: O=C1CCC(N2Cc3cc(OCCCl)ccc3C2=O)C(=O)N1] | | |
| | 7-82 | 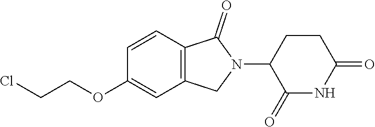 [SMILES: O=C1CCC(N2Cc3cc(OCCCl)ccc3C2=O)C(=O)N1] | | |
| | 7-83 | 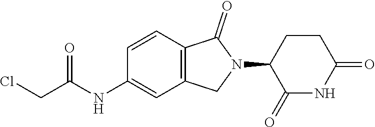 [SMILES: O=C1CC[C@H](N2Cc3cc(NC(=O)CCl)ccc3C2=O)C(=O)N1] | | |
| | 7-84 | 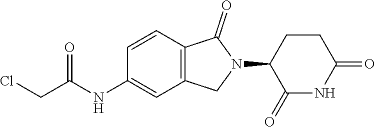 [SMILES: O=C1CC[C@H](N2Cc3cc(NC(=O)CCl)ccc3C2=O)C(=O)N1] | | |
| | 7-85 | 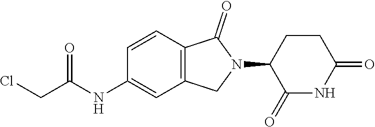 [SMILES: O=C1CC[C@H](N2Cc3cc(NC(=O)CCl)ccc3C2=O)C(=O)N1] | | |
| | 7-86 | 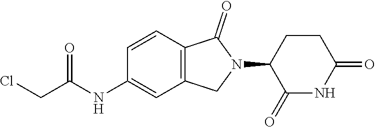 [SMILES: O=C1CC[C@H](N2Cc3cc(NC(=O)CCl)ccc3C2=O)C(=O)N1] | | |
| | 7-87 | 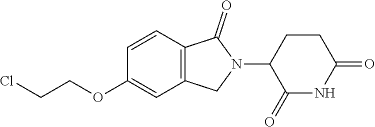 [SMILES: O=C1CCC(N2Cc3cc(OCCCl)ccc3C2=O)C(=O)N1] | | |
| | 7-88 | 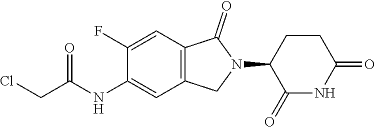 [SMILES: O=C1CC[C@H](N2Cc3cc(NC(=O)CCl)c(F)cc3C2=O)C(=O)N1] | | |
| | 7-89 | 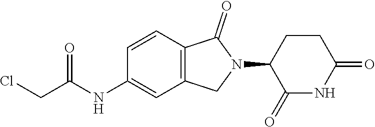 [SMILES: O=C1CC[C@H](N2Cc3cc(NC(=O)CCl)ccc3C2=O)C(=O)N1] | | |
| | 7-90 | 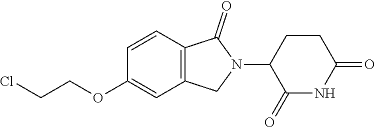 [SMILES: O=C1CCC(N2Cc3cc(OCCCl)ccc3C2=O)C(=O)N1] | | |
| | 7-91 | 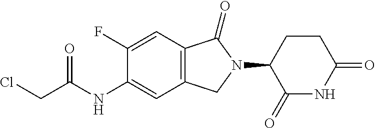 [SMILES: O=C1CC[C@H](N2Cc3cc(NC(=O)CCl)c(F)cc3C2=O)C(=O)N1] | | |
| | 7-93 | 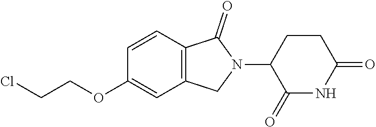 [SMILES: O=C1CCC(N2Cc3cc(OCCCl)ccc3C2=O)C(=O)N1] | | |
| | 7-94 | 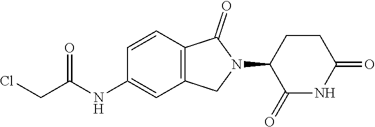 [SMILES: O=C1CC[C@H](N2Cc3cc(NC(=O)CCl)ccc3C2=O)C(=O)N1] | | |
| | 7-95 | 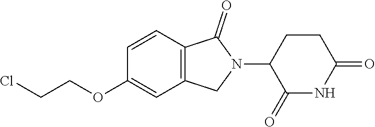 [SMILES: O=C1CCC(N2Cc3cc(OCCCl)ccc3C2=O)C(=O)N1] | | |
| | 7-96 | 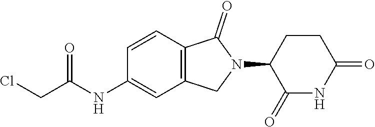 [SMILES: O=C1CC[C@H](N2Cc3cc(NC(=O)CCl)ccc3C2=O)C(=O)N1] | | |
| | 7-97 | 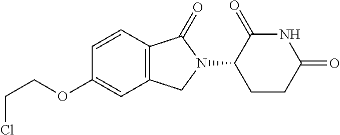 [SMILES: O=C1CC[C@H](N2Cc3cc(OCCCl)ccc3C2=O)C(=O)N1] | | |
| | 7-98 | 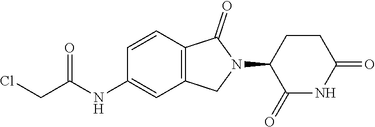 [SMILES: O=C1CC[C@H](N2Cc3cc(NC(=O)CCl)ccc3C2=O)C(=O)N1] | | |
| | 7-99 | 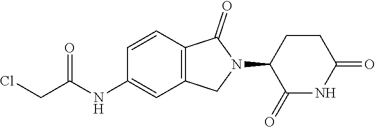 [SMILES: O=C1CC[C@H](N2Cc3cc(NC(=O)CCl)ccc3C2=O)C(=O)N1] | | |
| | 7-100 | 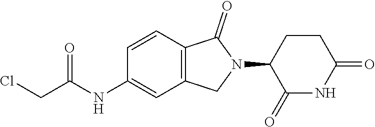 [SMILES: O=C1CC[C@H](N2Cc3cc(NC(=O)CCl)ccc3C2=O)C(=O)N1] | | |
| | 7-101 | 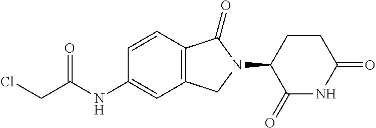 [SMILES: O=C1CC[C@H](N2Cc3cc(NC(=O)CCl)ccc3C2=O)C(=O)N1] | | |
| | 7-102 | 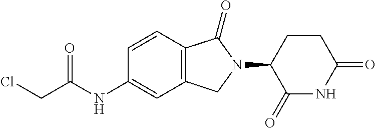 [SMILES: O=C1CC[C@H](N2Cc3cc(NC(=O)CCl)ccc3C2=O)C(=O)N1] | | |
| | 7-103 | 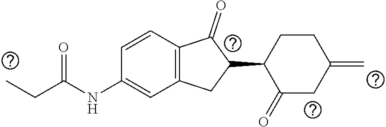 [SMILES: C=C1CC[C@H](C2Cc3cc(NC(=O)CC)ccc3C2=O)C(=O)C1] | | |
| | 7-104 | 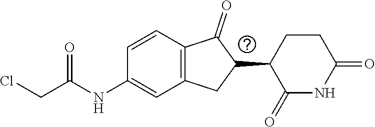 [SMILES: O=C1CC[C@H](C2Cc3cc(NC(=O)CCl)ccc3C2=O)C(=O)N1] | | |
| | 7-105 | 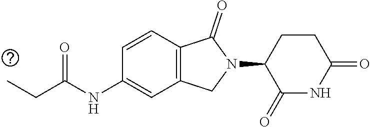 [SMILES: CCC(=O)Nc1ccc2c(c1)CN([C@H]1CCC(=O)NC1=O)C2=O] | | |
| | 7-106 | 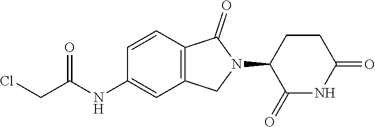 [SMILES: O=C1CC[C@H](N2Cc3cc(NC(=O)CCl)ccc3C2=O)C(=O)N1] | | |
| | 7-107 | 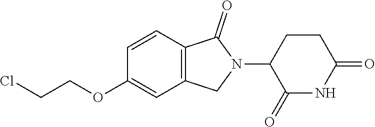 [SMILES: O=C1CCC(N2Cc3cc(OCCCl)ccc3C2=O)C(=O)N1] | | |
| | 7-108 | 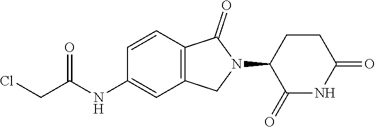 [SMILES: O=C1CC[C@H](N2Cc3cc(NC(=O)CCl)ccc3C2=O)C(=O)N1] | | |
| | 7-109 | 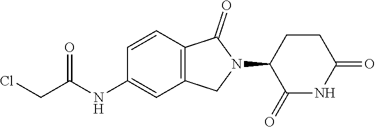 [SMILES: O=C1CC[C@H](N2Cc3cc(NC(=O)CCl)ccc3C2=O)C(=O)N1] | | |
| | 7-110 | 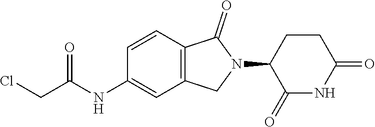 [SMILES: O=C1CC[C@H](N2Cc3cc(NC(=O)CCl)ccc3C2=O)C(=O)N1] | | |
| | 7-111 | 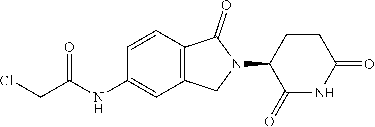 [SMILES: O=C1CC[C@H](N2Cc3cc(NC(=O)CCl)ccc3C2=O)C(=O)N1] | | |
| | 7-112 | 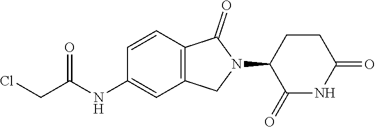 [SMILES: O=C1CC[C@H](N2Cc3cc(NC(=O)CCl)ccc3C2=O)C(=O)N1] | | |
| | 7-113 | 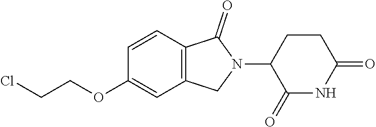 [SMILES: O=C1CCC(N2Cc3cc(OCCCl)ccc3C2=O)C(=O)N1] | | |
| | 7-114 | 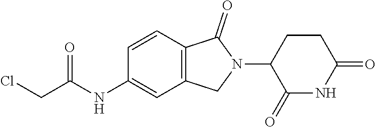 [SMILES: O=C1CCC(N2Cc3cc(NC(=O)CCl)ccc3C2=O)C(=O)N1] | | |
| | 7-115 | 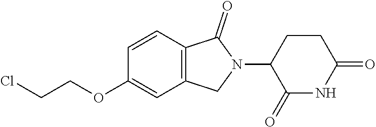 [SMILES: O=C1CCC(N2Cc3cc(OCCCl)ccc3C2=O)C(=O)N1] | | |
| | 7-116 | 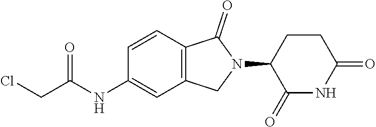 [SMILES: O=C1CC[C@H](N2Cc3cc(NC(=O)CCl)ccc3C2=O)C(=O)N1] | | |
| | 7-117 | 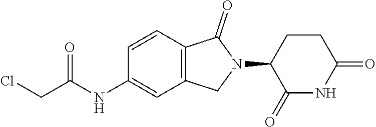 [SMILES: O=C1CC[C@H](N2Cc3cc(NC(=O)CCl)ccc3C2=O)C(=O)N1] | | |
| | | | Product | |
| | | Compd | 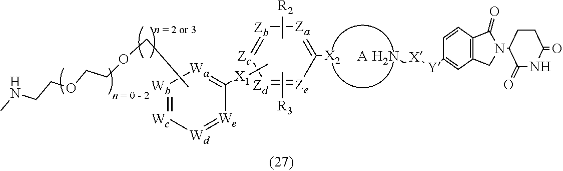 | MS found [M + H] + |
| | | 7-1 | 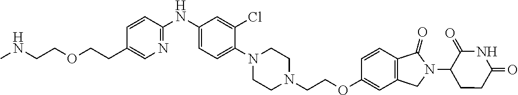 [SMILES: CNCCOCCc1ccc(Nc2ccc(N3CCN(CCOc4ccc5c(c4)CN(C4CCC(=O)NC4=O)C5=O)CC3)c(Cl)c2)nc1] | 676.3 |
| | | | 1H NMR (400 MHz, DMSO_d6) δ 2.12-2.27 (m, 1H), 2.41-2.58 | |
| | | | (m, 1H), 2.73 (s, 3H), 2.75-2.83 (m, 1H), 2.84-2.95 (m, 3H), 3.15- | |
| | | | 3.24 (m, 2H), 3.34 (d, J = 3.2 Hz, 2H), 3.46-3.58 (m, 3H), 3.58- | |
| | | | 3.67 (m, 2H), 3.70-3.88 (m, 8H), 4.42-4.53 (m, 2H), 4.54-4.65 | |
| | | | (m, 2H) ,5.07-5.18(m, 1H), 7.25 − 7.15 (m, 2H) , 7.29 (s, 1H), | |
| | | | 7.34-7.43 (m, 2H), 7.53 (d, J = 1.7 Hz, 1H), 7.78 (d, J = 8.5 Hz, | |
| | | | 1H), 7.84 (s, 1H), 8.08 (dd, J = 9.1, 1.9 Hz, 1H) | |
| | | 7-3 | 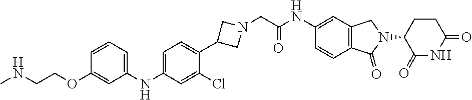 [SMILES: CNCCOc1cccc(Nc2ccc(C3CN(CC(=O)Nc4ccc5c(c4)CN([C@@H]4CCC(=O)NC4=O)C5=O)C3)c(Cl)c2)c1] | 631.4, 316.3 |
| | | 7-4 | 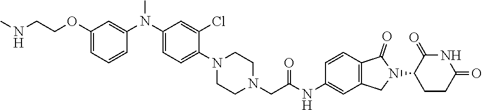 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 674.5, 337.8 |
| | | 7-5 | 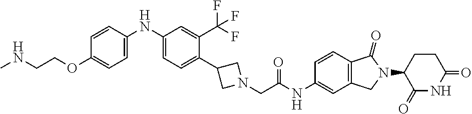 [SMILES: CNCCOc1ccc(Nc2ccc(C3CN(CC(=O)Nc4ccc5c(c4)CN([C@H]4CCC(=O)NC4=O)C5=O)C3)c(C(F)(F)F)c2)cc1] | 665.5, 333.3 |
| | | 7-6 | 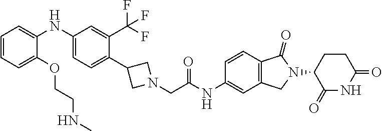 [SMILES: CNCCOc1ccccc1Nc1ccc(C2CN(CC(=O)Nc3ccc4c(c3)CN([C@@H]3CCC(=O)NC3=O)C4=O)C2)c(C(F)(F)F)c1] | 665.4, 333.3 |
| | | 7-7 | 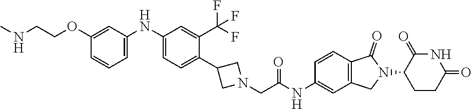 [SMILES: CNCCOc1cccc(Nc2ccc(C3CN(CC(=O)Nc4ccc5c(c4)CN([C@H]4CCC(=O)NC4=O)C5=O)C3)c(C(F)(F)F)c2)c1] | 665.4, 333.1 |
| | | 7-8 | 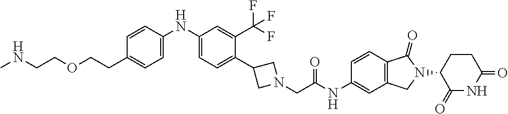 [SMILES: CNCCOCCc1ccc(Nc2ccc(C3CN(CC(=O)Nc4ccc5c(c4)CN([C@@H]4CCC(=O)NC4=O)C5=O)C3)c(C(F)(F)F)c2)cc1] | 694.5, 374.6 |
| | | 7-9 | 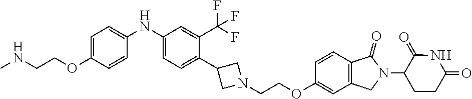 [SMILES: CNCCOc1ccc(Nc2ccc(C3CN(CCOc4ccc5c(c4)CN(C4CCC(=O)NC4=O)C5=O)C3)c(C(F)(F)F)c2)cc1] | 652.4, 326.6 |
| | | 7-10 | 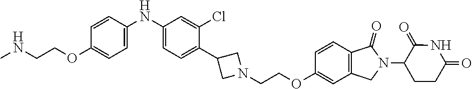 [SMILES: CNCCOc1ccc(Nc2ccc(C3CN(CCOc4ccc5c(c4)CN(C4CCC(=O)NC4=O)C5=O)C3)c(Cl)c2)cc1] | 309.8, 618.4 |
| | | 7-11 | 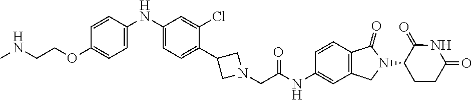 [SMILES: CNCCOc1ccc(Nc2ccc(C3CN(CC(=O)Nc4ccc5c(c4)CN([C@H]4CCC(=O)NC4=O)C5=O)C3)c(Cl)c2)cc1] | 631.3, 316.3 |
| | | 7-12 | 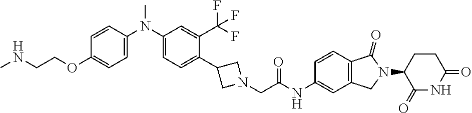 [SMILES: CNCCOc1ccc(N(C)c2ccc(C3CN(CC(=O)Nc4ccc5c(c4)CN([C@H]4CCC(=O)NC4=O)C5=O)C3)c(C(F)(F)F)c2)cc1] | 679.4, 340.3 |
| | | 7-13 | 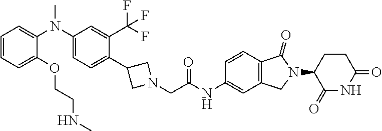 [SMILES: CNCCOc1ccccc1N(C)c1ccc(C2CN(CC(=O)Nc3ccc4c(c3)CN([C@H]3CCC(=O)NC3=O)C4=O)C2)c(C(F)(F)F)c1] | 679.5, 340.3 |
| | | 7-14 | 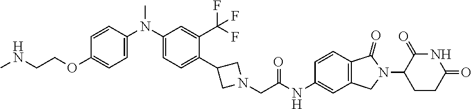 [SMILES: CNCCOc1ccc(N(C)c2ccc(C3CN(CC(=O)Nc4ccc5c(c4)CN(C4CCC(=O)NC4=O)C5=O)C3)c(C(F)(F)F)c2)cc1] | 666.3, 333.8 |
| | | 7-15 | 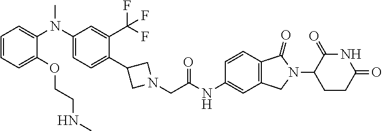 [SMILES: CNCCOc1ccccc1N(C)c1ccc(C2CN(CC(=O)Nc3ccc4c(c3)CN(C3CCC(=O)NC3=O)C4=O)C2)c(C(F)(F)F)c1] | 665.5, 332.7 |
| | | 7-16 | 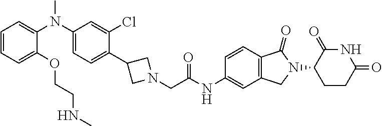 [SMILES: CNCCOc1ccccc1N(C)c1ccc(C2CN(CC(=O)Nc3ccc4c(c3)CN([C@H]3CCC(=O)NC3=O)C4=O)C2)c(Cl)c1] | 645.4, 323.1 |
| | | 7-17 | 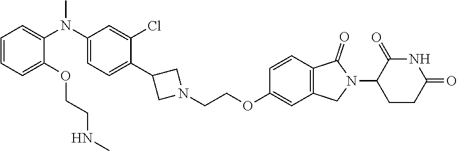 [SMILES: CNCCOc1ccccc1N(C)c1ccc(C2CN(CCOc3ccc4c(c3)CN(C3CCC(=O)NC3=O)C4=O)C2)c(Cl)c1] | 632.4, 316.7 |
| | | 7-18 | 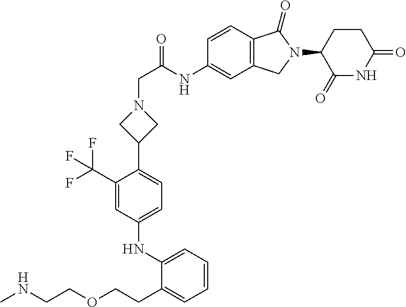 [SMILES: CNCCOCCc1ccccc1Nc1ccc(C2CN(CC(=O)Nc3ccc4c(c3)CN([C@H]3CCC(=O)NC3=O)C4=O)C2)c(C(F)(F)F)c1] | 693.5, 347.3 |
| | | 7-19 | 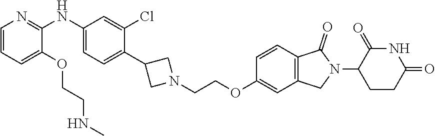 [SMILES: CNCCOc1cccnc1Nc1ccc(C2CN(CCOc3ccc4c(c3)CN(C3CCC(=O)NC3=O)C4=O)C2)c(Cl)c1] | 619.3, 310.1 |
| | | 7-20 | 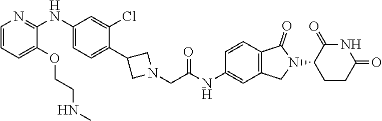 [SMILES: CNCCOc1cccnc1Nc1ccc(C2CN(CC(=O)Nc3ccc4c(c3)CN([C@H]3CCC(=O)NC3=O)C4=O)C2)c(Cl)c1] | 632.3, 316.7 |
| | | 7-21 | 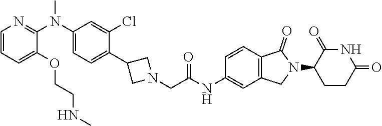 [SMILES: CNCCOc1cccnc1N(C)c1ccc(C2CN(CC(=O)Nc3ccc4c(c3)CN([C@@H]3CCC(=O)NC3=O)C4=O)C2)c(Cl)c1] | 646.4, 323.8 |
| | | 7-22 | 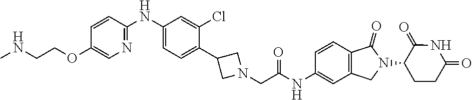 [SMILES: CNCCOc1ccc(Nc2ccc(C3CN(CC(=O)Nc4ccc5c(c4)CN([C@H]4CCC(=O)NC4=O)C5=O)C3)c(Cl)c2)nc1] | 632.2. 316.7 |
| | | 7-23 | 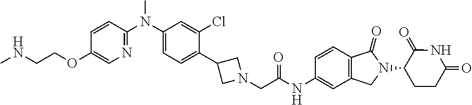 [SMILES: CNCCOc1ccc(N(C)c2ccc(C3CN(CC(=O)Nc4ccc5c(c4)CN([C@H]4CCC(=O)NC4=O)C5=O)C3)c(Cl)c2)nc1] | 646.5, 323.8 |
| | | 7-24 | 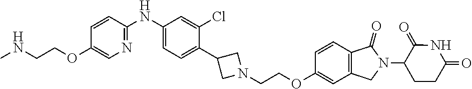 [SMILES: CNCCOc1ccc(Nc2ccc(C3CN(CCOc4ccc5c(c4)CN(C4CCC(=O)NC4=O)C5=O)C3)c(Cl)c2)nc1] | 619.4, 310.2 |
| | | 7-25 | 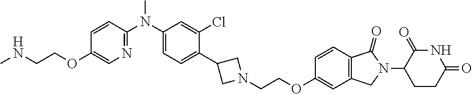 [SMILES: CNCCOc1ccc(N(C)c2ccc(C3CN(CCOc4ccc5c(c4)CN(C4CCC(=O)NC4=O)C5=O)C3)c(Cl)c2)nc1] | 633.4, 317.3 |
| | | 7-26 | 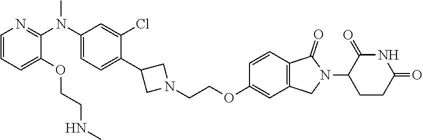 [SMILES: CNCCOc1cccnc1N(C)c1ccc(C2CN(CCOc3ccc4c(c3)CN(C3CCC(=O)NC3=O)C4=O)C2)c(Cl)c1] | 633.3, 317.2 |
| | | 7-27 | 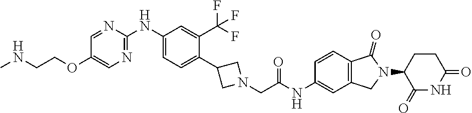 [SMILES: CNCCOc1cnc(Nc2ccc(C3CN(CC(=O)Nc4ccc5c(c4)CN([C@H]4CCC(=O)NC4=O)C5=O)C3)c(C(F)(F)F)c2)nc1] | 334.3, 667.5 |
| | | 7-28 | 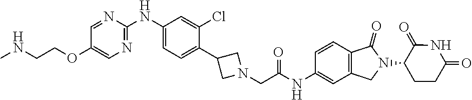 [SMILES: CNCCOc1cnc(Nc2ccc(C3CN(CC(=O)Nc4ccc5c(c4)CN([C@H]4CCC(=O)NC4=O)C5=O)C3)c(Cl)c2)nc1] | 633.3, 317.3 |
| | | 7-29 | 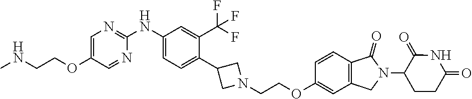 [SMILES: CNCCOc1cnc(Nc2ccc(C3CN(CCOc4ccc5c(c4)CN(C4CCC(=O)NC4=O)C5=O)C3)c(C(F)(F)F)c2)nc1] | 654.3, 327.8 |
| | | 7-30 | 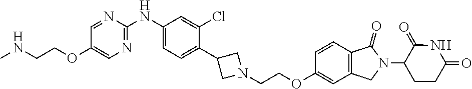 [SMILES: CNCCOc1cnc(Nc2ccc(C3CN(CCOc4ccc5c(c4)CN(C4CCC(=O)NC4=O)C5=O)C3)c(Cl)c2)nc1] | 616.0, 308.6 |
| | | 7-31 | 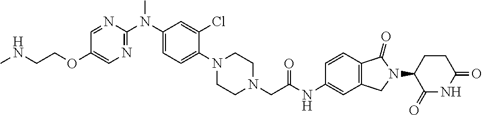 [SMILES: CNCCOc1cnc(N(C)c2ccc(N3CCN(CC(=O)Nc4ccc5c(c4)CN([C@H]4CCC(=O)NC4=O)C5=O)CC3)c(Cl)c2)nc1] | 676.3, 338.7 |
| | | | ¹H NMR (400 MHz, DMSO_d6) δ 2.11 − 2.22 (m, 1H) , 2.40 − | |
| | | | 2.55 (m, 1H), 2.69 − 2.84 (m, 4H), 2.85 − 2.95 (m, 1H), 3.32 − | |
| | | | 3.38 (m, 2H), 3.41 − 3.51 (m, 2H), 3.50 − 3.72 (m, 7H), 3.85 (d, | |
| | | | J = 11.4 Hz, 2H), 4.28 − 4.43 (m, 4H), 4.45 − 4.63 (m, 2H), 5.15 | |
| | | | (dd, J = 13.2, 5.2 Hz, 1H), 7.33 − 7.44 (m, 2H), 7.53 − 7.60 (m, | |
| | | | 1H), 7.64 − 7.72 (m, 1H), 7.73 − 7.82 (m, 1H), 8.04 (s, 1H), 8.40 (s, 2H). | |
| | | 7-32 | 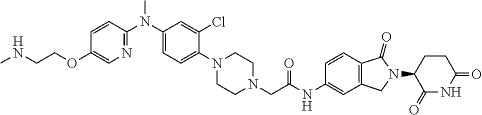 [SMILES: CNCCOc1ccc(N(C)c2ccc(N3CCN(CC(=O)Nc4ccc5c(c4)CN([C@H]4CCC(=O)NC4=O)C5=O)CC3)c(Cl)c2)nc1] | 675.1, 338.1 |
| | | | ¹H NMR (400 MHz, MeOD_d4) δ 2.12 − 2.22 (m, 1H), 2.41 − | |
| | | | 2.57 (m, 1H), 2.72 − 2.83 (m, 4H), 2.84 − 2.99 (m, 1H), 3.33 − | |
| | | | 3.41 (m, 2H), 3.45 − 3.61 (m, 7H), 3.66 (d, J = 12.8 Hz, 2H), | |
| | | | 3.87 (d, J = 11.2 Hz, 2H), 4.25 − 4.40 (m, 4H), 4.50 (q, J = 17.2 | |
| | | | Hz, 2H), 5.16 (dd, J = 13.6, 5.2 Hz, 1H), 7.08 (d, J = 10.0 Hz, | |
| | | | 1H), 7.39 − 7.48 (m, 2H), 7.61 (d, J = 2.0 Hz, 1H), 7.64 − 7.73 | |
| | | | (m, 2H), 7.80 (d, J = 8.4 Hz, 1H), 7.88 (dd, J = 10.0, 2.8 Hz, | |
| | | | 1H), 8.04 (s, 1H). | |
| | | 7-33 | 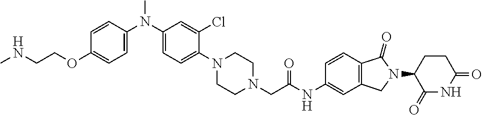 [SMILES: CNCCOc1ccc(N(C)c2ccc(N3CCN(CC(=O)Nc4ccc5c(c4)CN([C@H]4CCC(=O)NC4=O)C5=O)CC3)c(Cl)c2)cc1] | 675.3, 337.8 |
| | | | ¹H NMR (400 MHz, CD3OD) δ 2.11 − 2.22 (m, 1H) , 2.42 − | |
| | | | 2.56 (m, 1H), 2.70 − 2.82 (m, 4H), 2.85 − 2.93 (m, 1H), 3.11 − | |
| | | | 3.26 (m, 2H), 3.33 − 3.55 (m, 7H), 3.71 − 3.83 (m, 2H), 4.31 − | |
| | | | 4.23 (m, 3H), 4.40 − 4.55 (m, 2H), 5.09 − 5.17 (m, 1H), 6.81 − | |
| | | | 6.68 (m, 2H), 7.01 − 7.07 (m, 2H), 7.09 − 7.18 (m, 2H), 7.67 (d, | |
| | | | J = 7.6 Hz, 1H), 7.78 (d, J = 8.2 Hz, 1H), 8.04 (s, 1H). | |
| | | 7-34 | 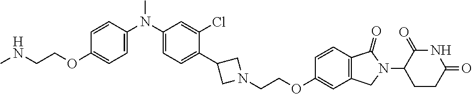 [SMILES: CNCCOc1ccc(N(C)c2ccc(C3CN(CCOc4ccc5c(c4)CN(C4CCC(=O)NC4=O)C5=O)C3)c(Cl)c2)cc1] | 632.5, 316.8 |
| | | 7-35 | 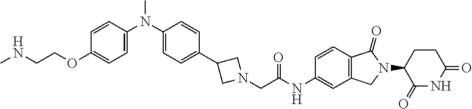 [SMILES: CNCCOc1ccc(N(C)c2ccc(C3CN(CC(=O)Nc4ccc5c(c4)CN([C@H]4CCC(=O)NC4=O)C5=O)C3)cc2)cc1] | 611.4, 306.2 |
| | | 7-36 | 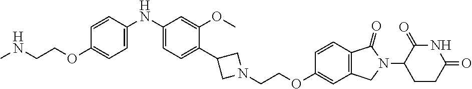 [SMILES: CNCCOc1ccc(Nc2ccc(C3CN(CCOc4ccc5c(c4)CN(C4CCC(=O)NC4=O)C5=O)C3)c(OC)c2)cc1] | 614.4, 307.7 |
| | | 7-37 | 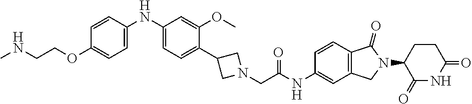 [SMILES: CNCCOc1ccc(Nc2ccc(C3CN(CC(=O)Nc4ccc5c(c4)CN([C@H]4CCC(=O)NC4=O)C5=O)C3)c(OC)c2)cc1] | 627.4, 314.2 |
| | | 7-38 | 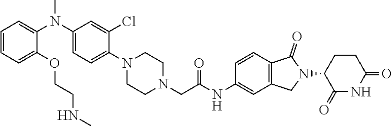 [SMILES: CNCCOc1ccccc1N(C)c1ccc(N2CCN(CC(=O)Nc3ccc4c(c3)CN([C@@H]3CCC(=O)NC3=O)C4=O)CC2)c(Cl)c1] | 675.3, 337.8 |
| | | | ¹H NMR (400 MHz, CD3OD) 2.12 − 2.20 (m, 1H), 2.42 − 2.53 | |
| | | | (m, 1H), 2.64 (s, 3H), 2.74 − 2.82 (m, 1H), 2.85 − 2.95 (m, 1H), | |
| | | | 3.10 − 3.27 (m, 6H), 3.33 − 3.42 (m, 2H), 3.43 − 3.53 (m, 2H), | |
| | | | 3.70 − 3.83 (m, 2H), 4.17 − 4.35 (m, 3H), 4.49 (q, J = 17.2 Hz, | |
| | | | 2H), 5.15 (dd, J = 13.2, 5.2 Hz, 1H), 6.56 − 6.65 (m, 1H), 6.70 | |
| | | | (d, J = 2.6 Hz, 1H), 7.02 − 7.13 (m, 2H), 7.15 − 7.24 (m, 2H), | |
| | | | 7.35 − 7.25 (m, 1H), 7.61 − 7.71 (m, 1H), 7.78 (d, J = 8.4 Hz, | |
| | | | 1H), 8.03 (s, 1H) | |
| | | 7-39 | 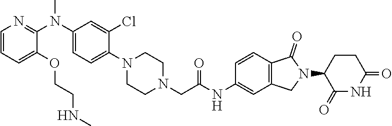 [SMILES: CNCCOc1cccnc1N(C)c1ccc(N2CCN(CC(=O)Nc3ccc4c(c3)CN([C@H]3CCC(=O)NC3=O)C4=O)CC2)c(Cl)c1] | 675.3, 338.3 |
| | | 7-40 | 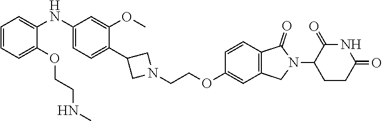 [SMILES: CNCCOc1ccccc1Nc1ccc(C2CN(CCOc3ccc4c(c3)CN(C3CCC(=O)NC3=O)C4=O)C2)c(OC)c1] | 628.3, 314.7 |
| | | 7-41 | 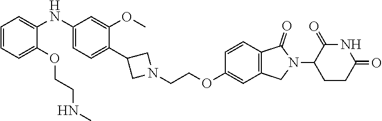 [SMILES: CNCCOc1ccccc1Nc1ccc(C2CN(CCOc3ccc4c(c3)CN(C3CCC(=O)NC3=O)C4=O)C2)c(OC)c1] | 614.2, 307.8 |
| | | 7-42 | 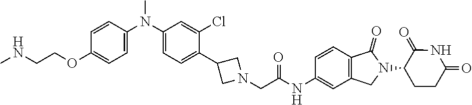 [SMILES: CNCCOc1ccc(N(C)c2ccc(C3CN(CC(=O)Nc4ccc5c(c4)CN([C@H]4CCC(=O)NC4=O)C5=O)C3)c(Cl)c2)cc1] | 646.2, 323.2 |
| | | 7-43 | 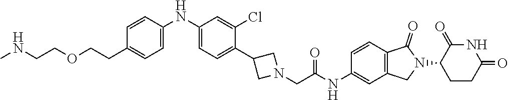 [SMILES: CNCCOCCc1ccc(Nc2ccc(C3CN(CC(=O)Nc4ccc5c(c4)CN([C@H]4CCC(=O)NC4=O)C5=O)C3)c(Cl)c2)cc1] | 659.4, 330.3 |
| | | 7-44 | 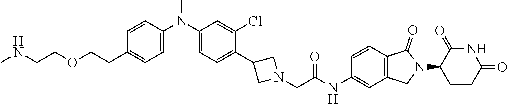 [SMILES: CNCCOCCc1ccc(N(C)c2ccc(C3CN(CC(=O)Nc4ccc5c(c4)CN([C@@H]4CCC(=O)NC4=O)C5=O)C3)c(Cl)c2)cc1] | 673.4, 337.3 |
| | | 7-45 | 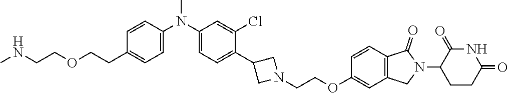 [SMILES: CNCCOCCc1ccc(N(C)c2ccc(C3CN(CCOc4ccc5c(c4)CN(C4CCC(=O)NC4=O)C5=O)C3)c(Cl)c2)cc1] | 331.8 |
| | | 7-46 | 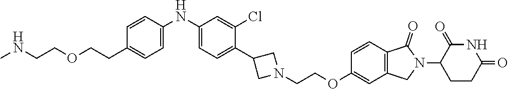 [SMILES: CNCCOCCc1ccc(Nc2ccc(C3CN(CCOc4ccc5c(c4)CN(C4CCC(=O)NC4=O)C5=O)C3)c(Cl)c2)cc1] | 646.2, 323.7 |
| | | 7-47 | 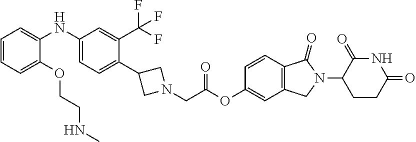 [SMILES: CNCCOc1ccccc1Nc1ccc(C2CN(CC(=O)Oc3ccc4c(c3)CN(C3CCC(=O)NC3=O)C4=O)C2)c(C(F)(F)F)c1] | 652.4, 326.8 |
| | | 7-48 | 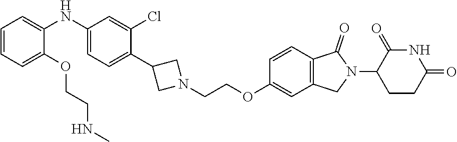 [SMILES: CNCCOc1ccccc1Nc1ccc(C2CN(CCOc3ccc4c(c3)CN(C3CCC(=O)NC3=O)C4=O)C2)c(Cl)c1] | 618.4, 309.8 |
| | | 7-49 | 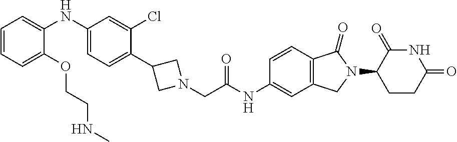 [SMILES: CNCCOc1ccccc1Nc1ccc(C2CN(CC(=O)Nc3ccc4c(c3)CN([C@@H]3CCC(=O)NC3=O)C4=O)C2)c(Cl)c1] | 631.3, 316.3 |
| | | 7-50 | 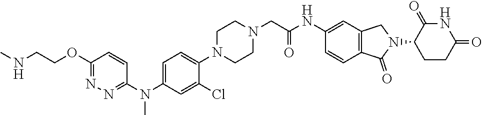 [SMILES: CNCCOc1ccc(N(C)c2ccc(N3CCN(CC(=O)Nc4ccc5c(c4)CN([C@H]4CCC(=O)NC4=O)C5=O)CC3)c(Cl)c2)nn1] | 338.9, 676.3 |
| | | 7-51 | 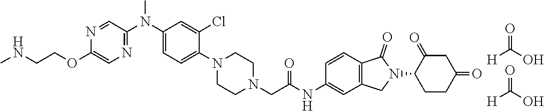 | 676.0, 338.6 |
| | | 7-52 | 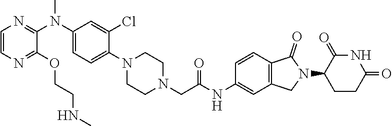 [SMILES: CNCCOc1nccnc1N(C)c1ccc(N2CCN(CC(=O)Nc3ccc4c(c3)CN([C@@H]3CCC(=O)NC3=O)C4=O)CC2)c(Cl)c1] | 676.0, 338.6 |
| | | 7-53 | 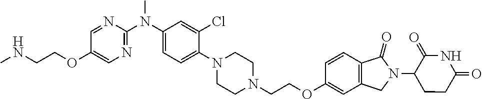 [SMILES: CNCCOc1cnc(N(C)c2ccc(N3CCN(CCOc4ccc5c(c4)CN(C4CCC(=O)NC4=O)C5=O)CC3)c(Cl)c2)nc1] | 662.9, 332.8 |
| | | | ¹H NMR (400 MHz, DMSO_d6) δ 1.98 − 2.01 (m, 1H), 2.32 − | |
| | | | 2.42 (m, 1H), 2.57 − 2.66 (m, 4H), 2.88 − 2.97 (m, 1H), 3.12 (s, | |
| | | | 2H), 3.31 − 3.42 (m, 9H), 3.72 (s, 4H), 4.23 − 4.26 (m, 2H), 4.28 − | |
| | | | 4.49 (m, 2H), 4.51 (s, 2H), 5.09 (dd, J = 13.2, 5.2 Hz, 1H), | |
| | | | 7.15 − 7.18 (m, 1H), 7.22 (d, J = 8.8 Hz, 1H), 7.28 − 7.31 (m, | |
| | | | 2H), 7.46 (d, J = 2.4 Hz, 1H), 7.70 (d, J = 8.4 Hz, 1H), 8.27 (s, | |
| | | | 2H), 8.72 (s, 2H), 10.18 (s, 1H), 10.99 (s, 1H). | |
| | | 7-54 | 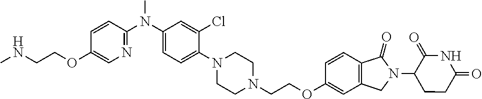 [SMILES: CNCCOc1ccc(N(C)c2ccc(N3CCN(CCOc4ccc5c(c4)CN(C4CCC(=O)NC4=O)C5=O)CC3)c(Cl)c2)nc1] | 662.0, 331.6 |
| | | | ¹H NMR (400 MHz, DMSO_d6) δ 1.95 − 2.03 (m, 1H), 2.10 (s, | |
| | | | 3H), 2.34 − 2.43 (m, 3H), 2.55 − 2.64 (m, 1H), 2.73 (s, 4H), 2.85 − | |
| | | | 2.96 (m, 1H), 3.04 (s, 4H), 3.27 (s, 2H), 3.31 (s, 4H), 4.07 (t, | |
| | | | J = 5.2 Hz, 2H), 4.37 (dd, J = 56.4, 17.2 Hz, 1H), 5.09 (dd, J = | |
| | | | 13.2, 5.0 Hz, 1H), 6.95 (dd, J = 8.6, 2.4 Hz, 1H), 7.07 − 7.18 (m, | |
| | | | 2H), 7.63 − 7.76 (m, 3H), 7.86 (d, J = 2.8 Hz, 1H), 8.03 (s, 1H), | |
| | | | 10.13 (s, 1H), 10.98 (s, 2H). | |
| | | 7-55 | 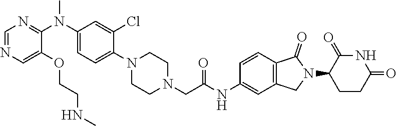 [SMILES: CNCCOc1cncnc1N(C)c1ccc(N2CCN(CC(=O)Nc3ccc4c(c3)CN([C@@H]3CCC(=O)NC3=O)C4=O)CC2)c(Cl)c1] | 338.9, 676.3 |
| | | | ¹H NMR (400 MHz, DMSO_d6) δ 1.97 − 2.05 (m, 1H), 2.31 (s, | |
| | | | 3H), 2.35 − 2.42 (m, 1H), 2.55 (s, 2H), 2.61 − 2.69 (m, 3H), 2.75 | |
| | | | (s, 4H), 2.86 − 2.97 (m, 2H), 3.07 (s, 4H), 3.32 − 3.34 (m, 2H), | |
| | | | 3.39 (s, 3H), 4.06 (t, J = 5.2 Hz, 2H), 4.30 (d, J = 17.2 Hz, 1H), | |
| | | | 4.45 (d, J = 17.2 Hz, 1H), 5.09 (dd, J = 12.8, 4.8 Hz, 1H), 7.08 − | |
| | | | 7.18 (m, 2H), 7.29 (d, J = 2.0 Hz, 1H), 7.67 (d, J = 8.4 Hz, 1H), | |
| | | | 7.78 (d, J = 8.4 Hz, 1H), 8.07 (s, 1H), 8.16 (s, 1H), 8.42 (s, 1H), | |
| | | | 9.34 (s, 1H), 10.48 (s, 1H), 10.99 (s, 1H). | |
| | | 7-56 | 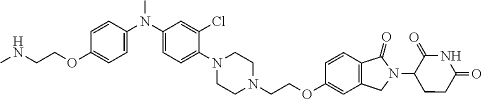 [SMILES: CNCCOc1ccc(N(C)c2ccc(N3CCN(CCOc4ccc5c(c4)CN(C4CCC(=O)NC4=O)C5=O)CC3)c(Cl)c2)cc1] | 331.0, 661.0 |
| | | | ¹H NMR (400 MHz, DMSO_d6) δ 1.97 − 2.03 (m, 1H), 2.30 − | |
| | | | 2.40 (m, 1H), 2.55 − 2.62 (m, 4H) 2.88 − 2.97 (m, 1H), 3.12 (s, | |
| | | | 2H), 3.31 − 3.42 (m, 9H), 3.72 (s, 4H), 4.24 (t, J = 5.2 Hz, 2H), | |
| | | | 4.30 − 4.47 (m, 2H), 4.51 (s, 2H), 5.09 (dd, J = 13.2, 5.2 Hz, 1H), | |
| | | | 6.70 (dd, J = 8.8, 2.8 Hz, 1H), 6.77 (d, J = 1.2 Hz, 1H), 6.98 − | |
| | | | 7.04 (m, 2H), 7.05 − 7.11 (m, 4H), 7.29 (s, 1H), 7.70 (d, J = 8.4 | |
| | | | Hz, 1H), 8.68 (s, 2H), 10.06 (s, 1H), 10.99 (s, 1H), | |
| | | 7-57 | 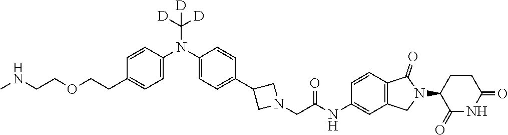 [SMILES: [2H]C([2H])([2H])N(c1ccc(CCOCCNC)cc1)c1ccc(C2CN(CC(=O)Nc3ccc4c(c3)CN([C@H]3CCC(=O)NC3=O)C4=O)C2)cc1] | 321.7 |
| | | 7-58 | 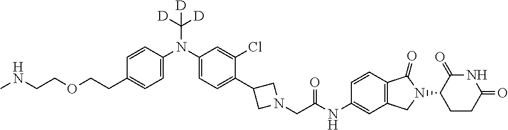 [SMILES: [2H]C([2H])([2H])N(c1ccc(CCOCCNC)cc1)c1ccc(C2CN(CC(=O)Nc3ccc4c(c3)CN([C@H]3CCC(=O)NC3=O)C4=O)C2)c(Cl)c1] | 337.8, 674.6 |
| | | 7-59 | 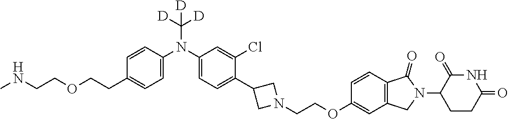 [SMILES: [2H]C([2H])([2H])N(c1ccc(CCOCCNC)cc1)c1ccc(C2CN(CCOc3ccc4c(c3)CN(C3CCC(=O)NC3=O)C4=O)C2)c(Cl)c1] | 331.2 |
| | | 7-60 | 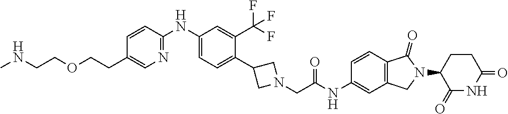 [SMILES: CNCCOCCc1ccc(Nc2ccc(C3CN(CC(=O)Nc4ccc5c(c4)CN([C@H]4CCC(=O)NC4=O)C5=O)C3)c(C(F)(F)F)c2)nc1] | 347.7 |
| | | 7-61 | 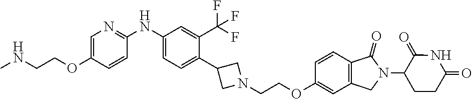 [SMILES: CNCCOc1ccc(Nc2ccc(C3CN(CCOc4ccc5c(c4)CN(C4CCC(=O)NC4=O)C5=O)C3)c(C(F)(F)F)c2)nc1] | 341.3 |
| | | 7-62 | 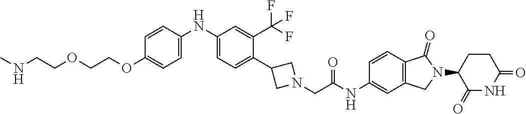 [SMILES: CNCCOCCOc1ccc(Nc2ccc(C3CN(CC(=O)Nc4ccc5c(c4)CN([C@H]4CCC(=O)NC4=O)C5=O)C3)c(C(F)(F)F)c2)cc1] | 709.4, 355.2 |
| | | 7-63 | 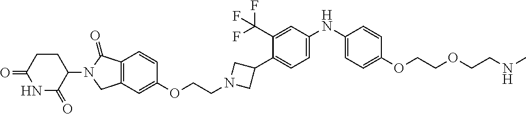 [SMILES: CNCCOCCOc1ccc(Nc2ccc(C3CN(CCOc4ccc5c(c4)CN(C4CCC(=O)NC4=O)C5=O)C3)c(C(F)(F)F)c2)cc1] | 348.8 |
| | | 7-64 | 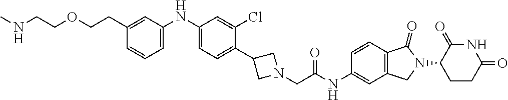 [SMILES: CNCCOCCc1cccc(Nc2ccc(C3CN(CC(=O)Nc4ccc5c(c4)CN([C@H]4CCC(=O)NC4=O)C5=O)C3)c(Cl)c2)c1] | 659.2, 330.1 |
| | | 7-65 | 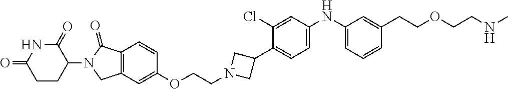 [SMILES: CNCCOCCc1cccc(Nc2ccc(C3CN(CCOc4ccc5c(c4)CN(C4CCC(=O)NC4=O)C5=O)C3)c(Cl)c2)c1] | 646.3, 323.6 |
| | | 7-66 | 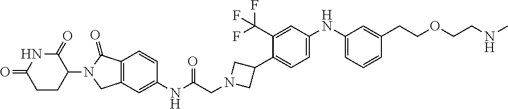 [SMILES: CNCCOCCc1cccc(Nc2ccc(C3CN(CC(=O)Nc4ccc5c(c4)CN(C4CCC(=O)NC4=O)C5=O)C3)c(C(F)(F)F)c2)c1] | 680.4, 340.7 |
| | | 7-67 | 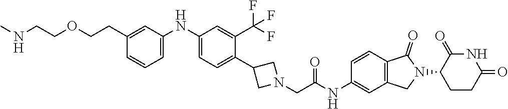 [SMILES: CNCCOCCc1cccc(Nc2ccc(C3CN(CC(=O)Nc4ccc5c(c4)CN([C@H]4CCC(=O)NC4=O)C5=O)C3)c(C(F)(F)F)c2)c1] | 693.4, 347.4 |
| | | 7-68 | 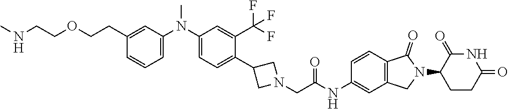 [SMILES: CNCCOCCc1cccc(N(C)c2ccc(C3CN(CC(=O)Nc4ccc5c(c4)CN([C@@H]4CCC(=O)NC4=O)C5=O)C3)c(C(F)(F)F)c2)c1] | 706.6, 354.2 |
| | | 7-69 | 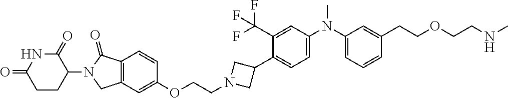 [SMILES: CNCCOCCc1cccc(N(C)c2ccc(C3CN(CCOc4ccc5c(c4)CN(C4CCC(=O)NC4=O)C5=O)C3)c(C(F)(F)F)c2)c1] | 694.5, 347.6 |
| | | 7-70 | 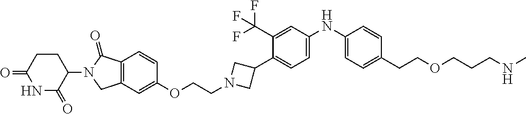 [SMILES: CNCCCOCCc1ccc(Nc2ccc(C3CN(CCOc4ccc5c(c4)CN(C4CCC(=O)NC4=O)C5=O)C3)c(C(F)(F)F)c2)cc1] | 694.4, 347.8 |
| | | 7-71 | 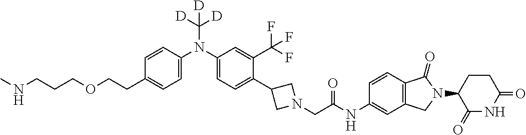 [SMILES: [2H]C([2H])([2H])N(c1ccc(CCOCCCNC)cc1)c1ccc(C2CN(CC(=O)Nc3ccc4c(c3)CN([C@H]3CCC(=O)NC3=O)C4=O)C2)c(C(F)(F)F)c1] | 724.5, 362.7 |
| | | 7-72 | 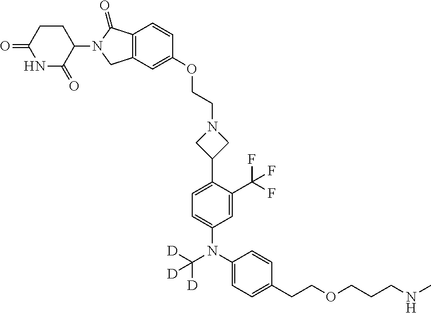 [SMILES: [2H]C([2H])([2H])N(c1ccc(CCOCCCNC)cc1)c1ccc(C2CN(CCOc3ccc4c(c3)CN(C3CCC(=O)NC3=O)C4=O)C2)c(C(F)(F)F)c1] | 711.5, 356.4 |
| | | 7-73 | 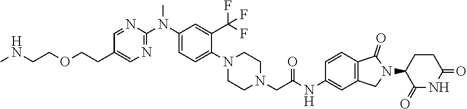 [SMILES: CNCCOCCc1cnc(N(C)c2ccc(N3CCN(CC(=O)Nc4ccc5c(c4)CN([C@H]4CCC(=O)NC4=O)C5=O)CC3)c(C(F)(F)F)c2)nc1] | 738.3, 369.8 |
| | | | ¹H NMR (400 MHz, DMSO_d6) δ 1.98 − 2.05 (m, 1H), 2.34 − | |
| | | | 2.41 (m, 1H), 2.56 − 2.65 (m, 1H), 2.71 (t, J = 6.2 Hz, 2H), 2.84 − | |
| | | | 2.98 (m, 1H), 3.02 − 3.18 (m, 4H), 3.24 − 3.45 (m, 4H), 3.48 | |
| | | | (s, 3H), 3.55 − 3.66 (m, 6H), 4.28 − 4.41 (m, 3H), 4.47 (d, J = | |
| | | | 17.8 Hz, 1H), 5.11 (dd, J = 13.2, 5.0 Hz, 1H), 7.60 (d, J = 8.8 | |
| | | | Hz, 1H), 7.66 − 7.78 (m, 4H), 7.96 (s, 1H), 8.34 (s, 1H), 8.56 (s, | |
| | | | 2H), 10.43 (s, 1H), 11.00 (s, 1H), 11.12 (s, 1H). | |
| | | 7-74 | 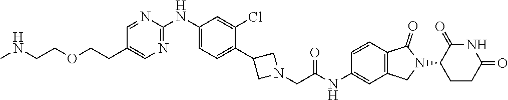 [SMILES: CNCCOCCc1cnc(Nc2ccc(C3CN(CC(=O)Nc4ccc5c(c4)CN([C@H]4CCC(=O)NC4=O)C5=O)C3)c(Cl)c2)nc1] | 661.2 |
| | | 7-75 | 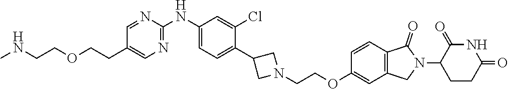 [SMILES: CNCCOCCc1cnc(Nc2ccc(C3CN(CCOc4ccc5c(c4)CN(C4CCC(=O)NC4=O)C5=O)C3)c(Cl)c2)nc1] | 648.5, 324.7 |
| | | 7-76 | 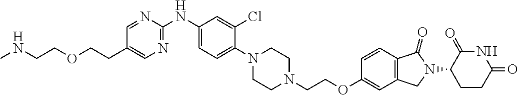 [SMILES: CNCCOCCc1cnc(Nc2ccc(N3CCN(CCOc4ccc5c(c4)CN([C@H]4CCC(=O)NC4=O)C5=O)CC3)c(Cl)c2)nc1] | 677.5, 339.2 |
| | | 7-77 | 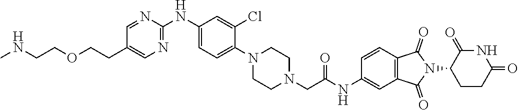 [SMILES: CNCCOCCc1cnc(Nc2ccc(N3CCN(CC(=O)Nc4ccc5c(c4)C(=O)N([C@H]4CCC(=O)NC4=O)C5=O)CC3)c(Cl)c2)nc1] | 690.4, 345.8 |
| | | 7-78 | 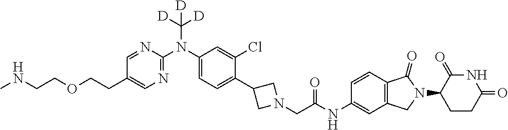 [SMILES: [2H]C([2H])([2H])N(c1ccc(C2CN(CC(=O)Nc3ccc4c(c3)CN([C@@H]3CCC(=O)NC3=O)C4=O)C2)c(Cl)c1)c1ncc(CCOCCNC)cn1] | 678.4, 339.6 |
| | | 7-79 | 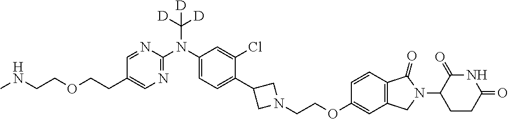 [SMILES: [2H]C([2H])([2H])N(c1ccc(C2CN(CCOc3ccc4c(c3)CN(C3CCC(=O)NC3=O)C4=O)C2)c(Cl)c1)c1ncc(CCOCCNC)cn1] | 665.5, 333.2 |
| | | 7-80 | 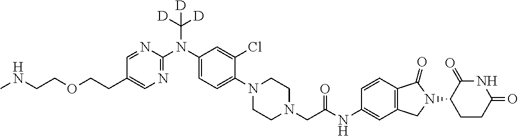 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(CCOCCNC)cn1] | 707.3 |
| | | 7-81 | 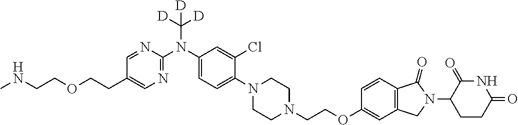 [SMILES: [2H]C([2H])([2H])N(c1ccc(N2CCN(CCOc3ccc4c(c3)CN(C3CCC(=O)NC3=O)C4=O)CC2)c(Cl)c1)c1ncc(CCOCCNC)cn1] | 694.3, 347.9 |
| | | 7-82 | 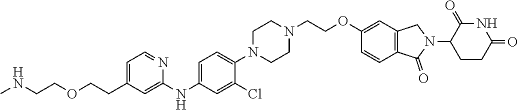 [SMILES: CNCCOCCc1ccnc(Nc2ccc(N3CCN(CCOc4ccc5c(c4)CN(C4CCC(=O)NC4=O)C5=O)CC3)c(Cl)c2)c1] | 677.4 |
| | | | ¹H NMR (400 MHz, MeOD) δ2.13-2.20 (m, 1H), 2.42-2.55 (m, | |
| | | | 1H), 2.68-2.81 (m, 4H), 2.85-2.95 (m, 1H), 3.04 (t, J = 6.0 Hz, | |
| | | | 2H), 3.18-3.24 (m, 2H), 3.31-3.38 (m, 2H), 3.53 (t, J = 12.0 Hz, | |
| | | | 2H), 3.61 (d, J = 13.2 Hz, 2H), 3.71-3.77 (m, 2H), 3.77-3.81 (m, | |
| | | | 2H), 3.81-3.89 (m, 4H), 4.48 (q, J = 17.2 Hz, 2H), 4.55-4.63 (m, | |
| | | | 2H), 5.12 (dd, J = 13.2, 5.2 Hz, 1H), 7.05 (d, J = 6.4 Hz, 1H), | |
| | | | 7.16-7.23 (m, 2H), 7.28 (s, 1H), 7.38 (s, 2H), 7.53 (d, J = 1.6 Hz, | |
| | | | 1H), 7.73-7.82 (m, 2H). | |
| | | 7-83 | 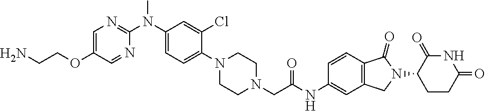 [SMILES: CN(c1ccc(N2CCN(CC(=O)Nc3ccc4c(c3)CN([C@H]3CCC(=O)NC3=O)C4=O)CC2)c(Cl)c1)c1ncc(OCCN)cn1] | 331.6, 663.3 |
| | | | ¹H NMR (400 MHz, CD3OD) δ 2.12 − 2.22 (m, 1H), 2.40 − 2.57 | |
| | | | (m, 1H), 2.73 − 2.83 (m, 1H), 2.85 − 2.97 (m, 1H), 3.32 − 3.45 | |
| | | | (m, 4H), 3.45 − 3.69 (m, 7H), 3.86 (d, J = 11.2 Hz, 2H), 4.22 − | |
| | | | 4.42 (m, 4H), 4.50 (q, J = 17.2 Hz, 2H), 5.15 (dd, J = 13.4, 5.2 | |
| | | | Hz, 1H), 7.29 − 7.44 (m, 2H), 7.50 − 7.59 (m, 1H), 7.64 − 7.72 | |
| | | | (m, 1H), 7.79 (d, J = 8.4 Hz, 1H), 8.04 (s, 1H), 8.39 (s, 2H). | |
| | | 7-84 | 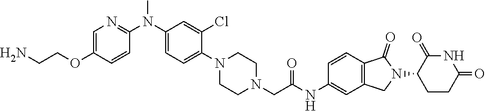 [SMILES: CN(c1ccc(N2CCN(CC(=O)Nc3ccc4c(c3)CN([C@H]3CCC(=O)NC3=O)C4=O)CC2)c(Cl)c1)c1ccc(OCCN)cn1] | 661.0, 331.1 |
| | | | ¹H NMR (400 MHz, DMSO_d6) δ 1.95 − 2.06 (m, 1H), 2.33 − | |
| | | | 2.44 (m, 1H), 2.53 − 2.66 (m, 1H), 2.85 − 3.01(m, 1H), 3.10 − | |
| | | | 3.31 (m, 4H), 3.33 − 3.55 (m, 7H), 3.70 (d, J = 10.8 Hz, 2H), | |
| | | | 4.20 (t, J = 4.8 Hz, 2H), 4.28 − 4.51 (m, 4H), 5.10 (dd, J = 13.2, | |
| | | | 5.0 Hz, 1H), 6.78 (d, J = 9.4 Hz, 1H), 7.21 − 7.32 (m, 2H), 7.39 − | |
| | | | 7.56 (m, 2H), 7.74 (s, 2H), 7.91 (d, J = 2.6 Hz, 1H), 7.98 (s, 1H), | |
| | | | 8.25 (s, 3H), 10.75 (s, 1H), 11.00 (s, 1H), 11.37 (s, 1H). | |
| | | 7-85 | 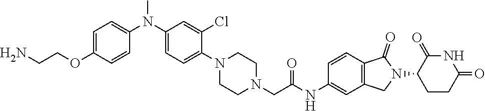 [SMILES: CN(c1ccc(OCCN)cc1)c1ccc(N2CCN(CC(=O)Nc3ccc4c(c3)CN([C@H]3CCC(=O)NC3=O)C4=O)CC2)c(Cl)c1] | 330.6, 659.9 |
| | | 7-86 | 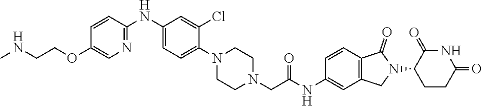 [SMILES: CNCCOc1ccc(Nc2ccc(N3CCN(CC(=O)Nc4ccc5c(c4)CN([C@H]4CCC(=O)NC4=O)C5=O)CC3)c(Cl)c2)nc1] | 661.6, 663.4 |
| | | 7-87 | 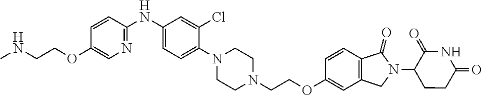 [SMILES: CNCCOc1ccc(Nc2ccc(N3CCN(CCOc4ccc5c(c4)CN(C4CCC(=O)NC4=O)C5=O)CC3)c(Cl)c2)nc1] | 648.3, 650.5 |
| | | 7-88 | 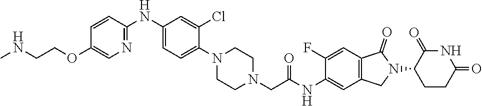 [SMILES: CNCCOc1ccc(Nc2ccc(N3CCN(CC(=O)Nc4cc5c(cc4F)C(=O)N([C@H]4CCC(=O)NC4=O)C5)CC3)c(Cl)c2)nc1] | 679.4, 681.4 |
| | | 7-89 | 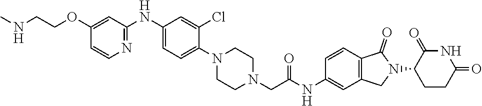 [SMILES: CNCCOc1ccnc(Nc2ccc(N3CCN(CC(=O)Nc4ccc5c(c4)CN([C@H]4CCC(=O)NC4=O)C5=O)CC3)c(Cl)c2)c1] | 661.5, 663.4 |
| | | 7-90 | 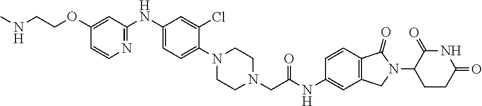 [SMILES: CNCCOc1ccnc(Nc2ccc(N3CCN(CC(=O)Nc4ccc5c(c4)CN(C4CCC(=O)NC4=O)C5=O)CC3)c(Cl)c2)c1] | 649.2, 651.2 |
| | | 7-91 | 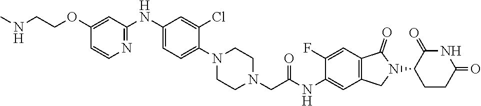 [SMILES: CNCCOc1ccnc(Nc2ccc(N3CCN(CC(=O)Nc4cc5c(cc4F)C(=O)N([C@H]4CCC(=O)NC4=O)C5)CC3)c(Cl)c2)c1] | 680.3, 682.2 |
| | | 7-93 | 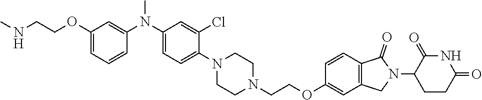 [SMILES: CNCCOc1cccc(N(C)c2ccc(N3CCN(CCOc4ccc5c(c4)CN(C4CCC(=O)NC4=O)C5=O)CC3)c(Cl)c2)c1] | 661.5, 663.5 |
| | | 7-94 | 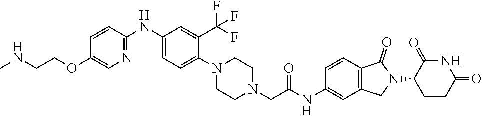 [SMILES: CNCCOc1ccc(Nc2ccc(N3CCN(CC(=O)Nc4ccc5c(c4)CN([C@H]4CCC(=O)NC4=O)C5=O)CC3)c(C(F)(F)F)c2)nc1] | 709.4 |
| | | 7-95 | 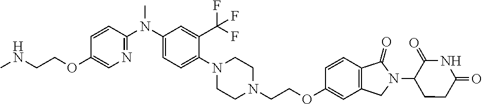 [SMILES: CNCCOc1ccc(N(C)c2ccc(N3CCN(CCOc4ccc5c(c4)CN(C4CCC(=O)NC4=O)C5=O)CC3)c(C(F)(F)F)c2)nc1] | 696.4 |
| | | 7-96 | 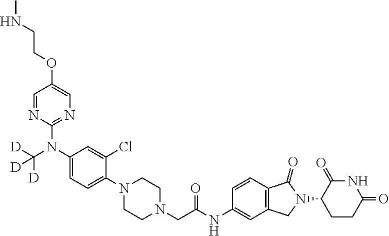 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCNC)cn1] | 679.4 |
| | | 7-97 | 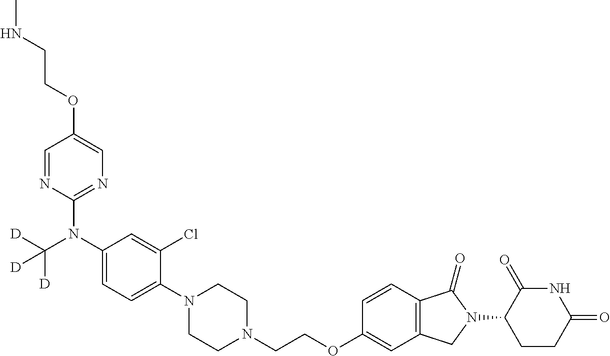 [SMILES: [2H]C([2H])([2H])N(c1ccc(N2CCN(CCOc3ccc4c(c3)CN([C@H]3CCC(=O)NC3=O)C4=O)CC2)c(Cl)c1)c1ncc(OCCNC)cn1] | 666.5 |
| | | 7-98 | 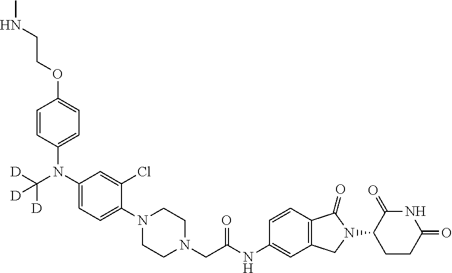 [SMILES: [2H]C([2H])([2H])N(c1ccc(OCCNC)cc1)c1ccc(N2CCN(CC(=O)Nc3ccc4c(c3)CN([C@H]3CCC(=O)NC3=O)C4=O)CC2)c(Cl)c1] | 677.4 |
| | | 7-99 | 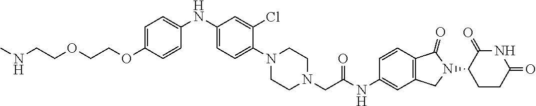 [SMILES: CNCCOCCOc1ccc(Nc2ccc(N3CCN(CC(=O)Nc4ccc5c(c4)CN([C@H]4CCC(=O)NC4=O)C5=O)CC3)c(Cl)c2)cc1] | 704.4 |
| | | 7-100 | 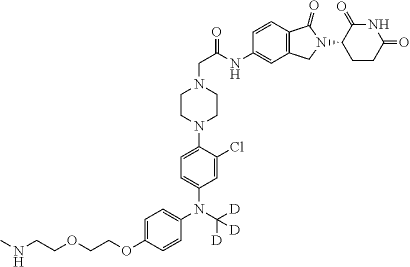 [SMILES: [2H]C([2H])([2H])N(c1ccc(OCCOCCNC)cc1)c1ccc(N2CCN(CC(=O)Nc3ccc4c(c3)CN([C@H]3CCC(=O)NC3=O)C4=O)CC2)c(Cl)c1] | 721.4 |
| | | 7-101 | 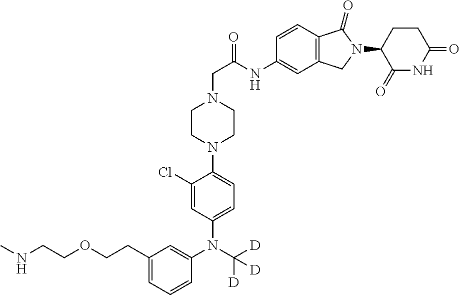 [SMILES: [2H]C([2H])([2H])N(c1cccc(CCOCCNC)c1)c1ccc(N2CCN(CC(=O)Nc3ccc4c(c3)CN([C@H]3CCC(=O)NC3=O)C4=O)CC2)c(Cl)c1] | 705.4 |
| | | 7-102 | 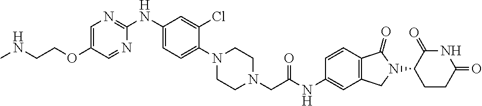 [SMILES: CNCCOc1cnc(Nc2ccc(N3CCN(CC(=O)Nc4ccc5c(c4)CN([C@H]4CCC(=O)NC4=O)C5=O)CC3)c(Cl)c2)nc1] | 662.5 |
| | | 7-103 | 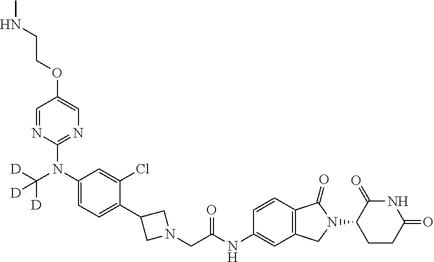 [SMILES: [2H]C([2H])([2H])N(c1ccc(C2CN(CC(=O)Nc3ccc4c(c3)CN([C@H]3CCC(=O)NC3=O)C4=O)C2)c(Cl)c1)c1ncc(OCCNC)cn1] | 650.6 |
| | | 7-104 | 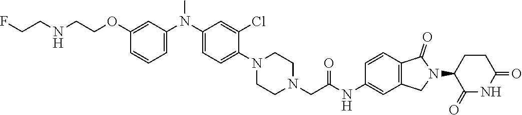 [SMILES: CN(c1cccc(OCCNCCF)c1)c1ccc(N2CCN(CC(=O)Nc3ccc4c(c3)CN([C@H]3CCC(=O)NC3=O)C4=O)CC2)c(Cl)c1] | 706.4 |
| | | 7-105 | 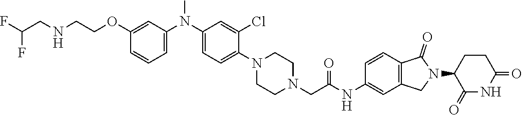 [SMILES: CN(c1cccc(OCCNCC(F)F)c1)c1ccc(N2CCN(CC(=O)Nc3ccc4c(c3)CN([C@H]3CCC(=O)NC3=O)C4=O)CC2)c(Cl)c1] | 724.4 |
| | | 7-106 | 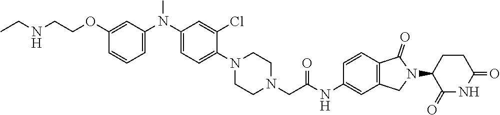 [SMILES: CCNCCOc1cccc(N(C)c2ccc(N3CCN(CC(=O)Nc4ccc5c(c4)CN([C@H]4CCC(=O)NC4=O)C5=O)CC3)c(Cl)c2)c1] | 688.4 |
| | | 7-107 | 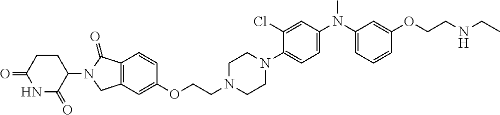 [SMILES: CCNCCOc1cccc(N(C)c2ccc(N3CCN(CCOc4ccc5c(c4)CN(C4CCC(=O)NC4=O)C5=O)CC3)c(Cl)c2)c1] | 675.4 |
| | | 7-108 | 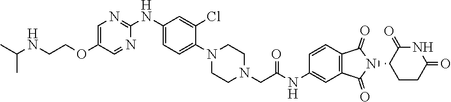 [SMILES: CC(C)NCCOc1cnc(Nc2ccc(N3CCN(CC(=O)Nc4ccc5c(c4)C(=O)N([C@H]4CCC(=O)NC4=O)C5=O)CC3)c(Cl)c2)nc1] | 688.4 |
| | | 7-109 | 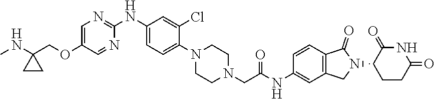 [SMILES: CNC1(COc2cnc(Nc3ccc(N4CCN(CC(=O)Nc5ccc6c(c5)CN([C@H]5CCC(=O)NC5=O)C6=O)CC4)c(Cl)c3)nc2)CC1] | 688.4 |
| | | 7-110 | 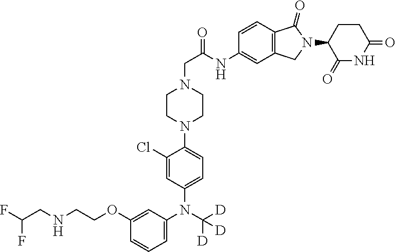 [SMILES: [2H]C([2H])([2H])N(c1cccc(OCCNCC(F)F)c1)c1ccc(N2CCN(CC(=O)Nc3ccc4c(c3)CN([C@H]3CCC(=O)NC3=O)C4=O)CC2)c(Cl)c1] | 727.5 |
| | | 7-111 | 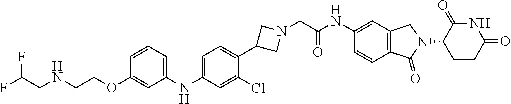 [SMILES: O=C1CC[C@H](N2Cc3cc(NC(=O)CN4CC(c5ccc(Nc6cccc(OCCNCC(F)F)c6)cc5Cl)C4)ccc3C2=O)C(=O)N1] | 681.4 |
| | | 7-112 | 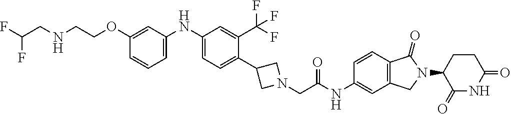 [SMILES: O=C1CC[C@H](N2Cc3cc(NC(=O)CN4CC(c5ccc(Nc6cccc(OCCNCC(F)F)c6)cc5C(F)(F)F)C4)ccc3C2=O)C(=O)N1] | 715.4 |
| | | 7-113 | 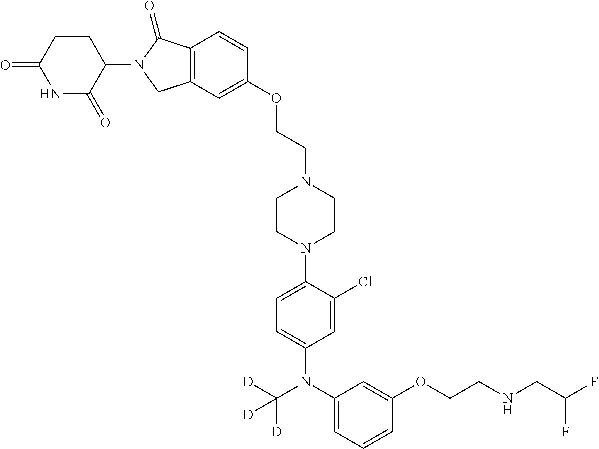 [SMILES: [2H]C([2H])([2H])N(c1cccc(OCCNCC(F)F)c1)c1ccc(N2CCN(CCOc3ccc4c(c3)CN(C3CCC(=O)NC3=O)C4=O)CC2)c(Cl)c1] | 714.4 |
| | | 7-114 | 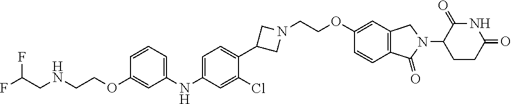 [SMILES: O=C1CCC(N2Cc3cc(OCCN4CC(c5ccc(Nc6cccc(OCCNCC(F)F)c6)cc5Cl)C4)ccc3C2=O)C(=O)N1] | 668.4 |
| | | 7-115 | 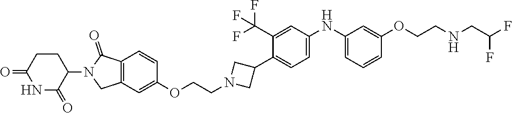 [SMILES: O=C1CCC(N2Cc3cc(OCCN4CC(c5ccc(Nc6cccc(OCCNCC(F)F)c6)cc5C(F)(F)F)C4)ccc3C2=O)C(=O)N1] | 702.6 |
| | | 7-116 | 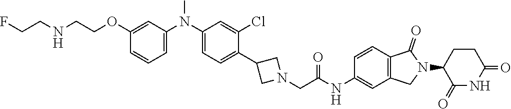 [SMILES: CN(c1cccc(OCCNCCF)c1)c1ccc(C2CN(CC(=O)Nc3ccc4c(c3)CN([C@H]3CCC(=O)NC3=O)C4=O)C2)c(Cl)c1] | 677.4 |
| | | 7-117 | 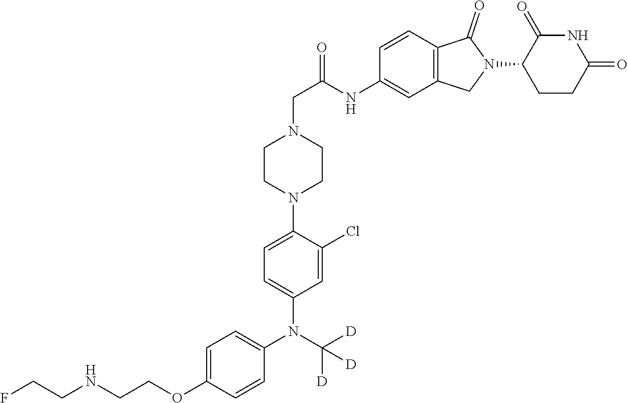 [SMILES: [2H]C([2H])([2H])N(c1ccc(OCCNCCF)cc1)c1ccc(N2CCN(CC(=O)Nc3ccc4c(c3)CN([C@H]3CCC(=O)NC3=O)C4=O)CC2)c(Cl)c1] | 709.4 |

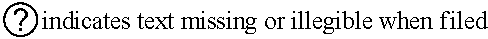

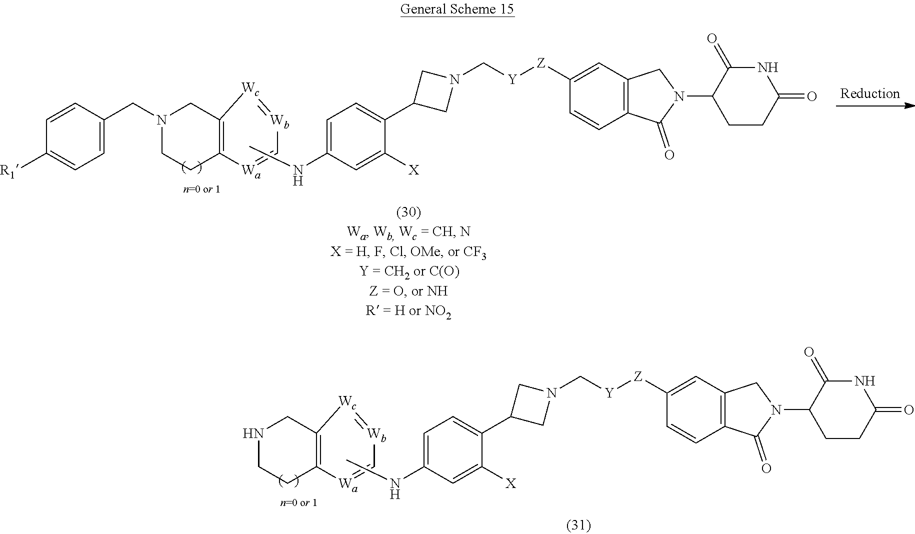

### Method 61

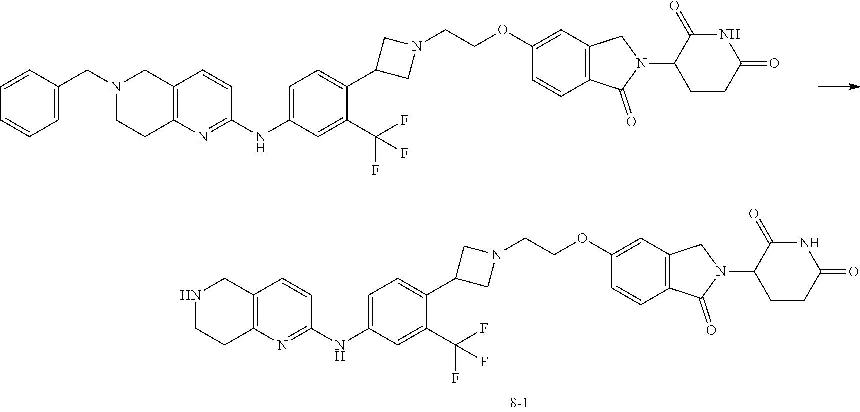 [SMILES: O=C1CCC(N2Cc3cc(OCCN4CC(c5ccc(Nc6ccc7c(n6)CCN(Cc6ccccc6)C7)cc5C(F)(F)F)C4)ccc3C2=O)C(=O)N1 ; O=C1CCC(N2Cc3cc(OCCN4CC(c5ccc(Nc6ccc7c(n6)CCNC7)cc5C(F)(F)F)C4)ccc3C2=O)C(=O)N1]

A mixture of 3-[5-[2-[3-[4-[(6-benzyl-7,8-dihydro-5H-1,6-naphthyridin-2-yl)amino]-2-(trifluoromethyl)phenyl]azetidin-1-yl]ethoxy]-1-oxo-isoindolin-2-yl]piperidine-2,6-dione (30 mg, 39.41 μmol, CL) and Palladium, 10% on carbon, Type 487, dry (419.40 μg, 3.94 μmol) in MeOH (5 mL) was equipped hydrogen gas ballon and stirred for 1 hour at a room temperature. The mixture was filtered through a celite filter, and the filtrate was collected, concentrated, and purified by a flash column. MeOH 0 to 40% gradient in EtOAc. ESI-MS: m/z=676.3 [M+1]⁺.

The following compounds 8-1 through 8-14 (31) shown in Table 8 were prepared by methods similar to those described for the preparation of 8-1, using the appropriate Benzylamine intermediate (30).

TABLE 8

| | | Product | |
|---|---|---|---|
| Compd | 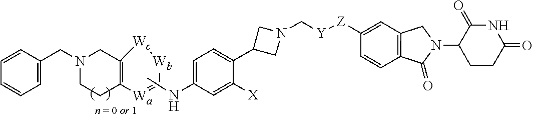 | 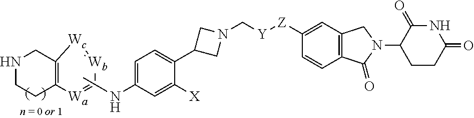 | MS found [M + H]+ |
| 8-1 | 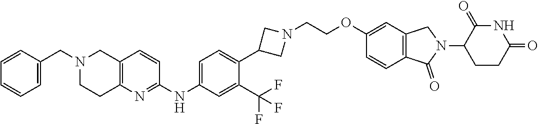 [SMILES: O=C1CCC(N2Cc3cc(OCCN4CC(c5ccc(Nc6ccc7c(n6)CCN(Cc6ccccc6)C7)cc5C(F)(F)F)C4)ccc3C2=O)C(=O)N1] | 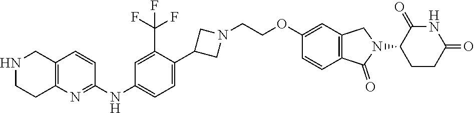 [SMILES: O=C1CC[C@H](N2Cc3cc(OCCN4CC(c5ccc(Nc6ccc7c(n6)CCNC7)cc5C(F)(F)F)C4)ccc3C2=O)C(=O)N1] | 635.6, 318.3 |
| 8-2 | 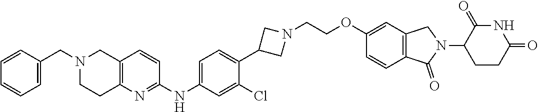 [SMILES: O=C1CCC(N2Cc3cc(OCCN4CC(c5ccc(Nc6ccc7c(n6)CCN(Cc6ccccc6)C7)cc5Cl)C4)ccc3C2=O)C(=O)N1] | 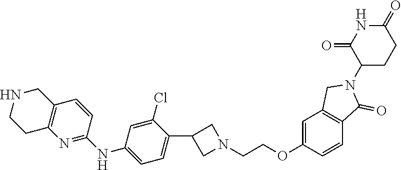 [SMILES: O=C1CCC(N2Cc3cc(OCCN4CC(c5ccc(Nc6ccc7c(n6)CCNC7)cc5Cl)C4)ccc3C2=O)C(=O)N1] | 600.4, 300.3 |
| 8-3 | 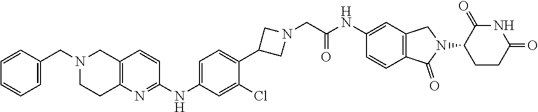 [SMILES: O=C1CC[C@H](N2Cc3cc(NC(=O)CN4CC(c5ccc(Nc6ccc7c(n6)CCN(Cc6ccccc6)C7)cc5Cl)C4)ccc3C2=O)C(=O)N1] | 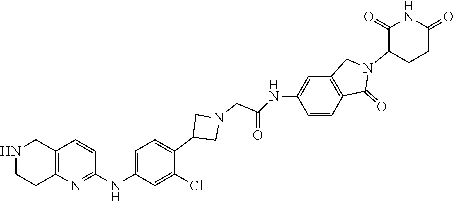 [SMILES: O=C1CCC(N2Cc3cc(NC(=O)CN4CC(c5ccc(Nc6ccc7c(n6)CCNC7)cc5Cl)C4)ccc3C2=O)C(=O)N1] | 614.4 |
| 8-4 | 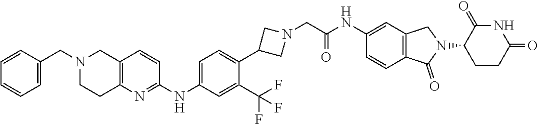 [SMILES: O=C1CC[C@H](N2Cc3cc(NC(=O)CN4CC(c5ccc(Nc6ccc7c(n6)CCN(Cc6ccccc6)C7)cc5C(F)(F)F)C4)ccc3C2=O)C(=O)N1] | 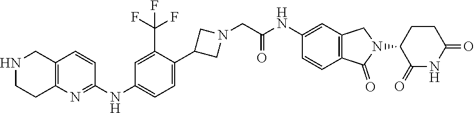 [SMILES: O=C1CC[C@@H](N2Cc3cc(NC(=O)CN4CC(c5ccc(Nc6ccc7c(n6)CCNC7)cc5C(F)(F)F)C4)ccc3C2=O)C(=O)N1] | 648.4, 324.8 |
| 8 .-5 | 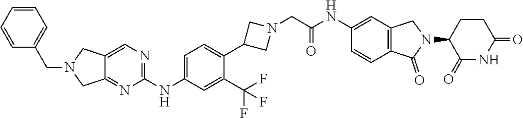 [SMILES: O=C1CC[C@H](N2Cc3cc(NC(=O)CN4CC(c5ccc(Nc6ncc7c(n6)CN(Cc6ccccc6)C7)cc5C(F)(F)F)C4)ccc3C2=O)C(=O)N1] | 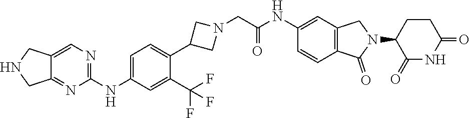 [SMILES: O=C1CC[C@H](N2Cc3cc(NC(=O)CN4CC(c5ccc(Nc6ncc7c(n6)CNC7)cc5C(F)(F)F)C4)ccc3C2=O)C(=O)N1] | 635.3, 318.0 |
| 8-6 | 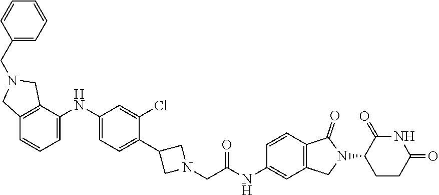 [SMILES: O=C1CC[C@H](N2Cc3cc(NC(=O)CN4CC(c5ccc(Nc6cccc7c6CN(Cc6ccccc6)C7)cc5Cl)C4)ccc3C2=O)C(=O)N1] | 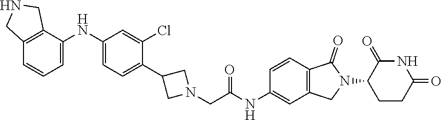 [SMILES: O=C1CC[C@H](N2Cc3cc(NC(=O)CN4CC(c5ccc(Nc6cccc7c6CNC7)cc5Cl)C4)ccc3C2=O)C(=O)N1] | 599.2, 300.2 |
| 8-7 | 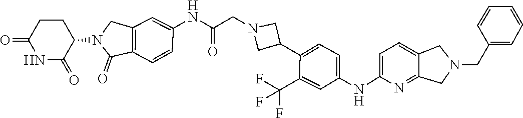 [SMILES: O=C1CC[C@H](N2Cc3cc(NC(=O)CN4CC(c5ccc(Nc6ccc7c(n6)CN(Cc6ccccc6)C7)cc5C(F)(F)F)C4)ccc3C2=O)C(=O)N1] | 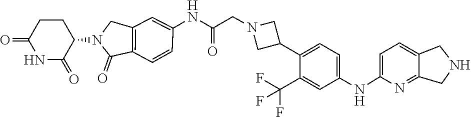 [SMILES: O=C1CC[C@H](N2Cc3cc(NC(=O)CN4CC(c5ccc(Nc6ccc7c(n6)CNC7)cc5C(F)(F)F)C4)ccc3C2=O)C(=O)N1] | 634.3, 617.6 |
| 8-8 | 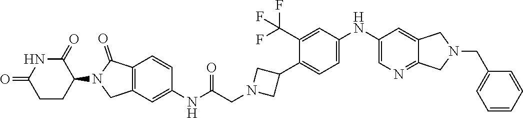 [SMILES: O=C1CC[C@H](N2Cc3cc(NC(=O)CN4CC(c5ccc(Nc6cnc7c(c6)CN(Cc6ccccc6)C7)cc5C(F)(F)F)C4)ccc3C2=O)C(=O)N1] | 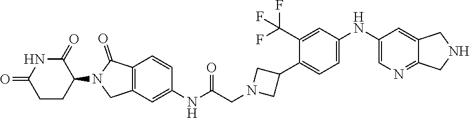 [SMILES: O=C1CC[C@H](N2Cc3cc(NC(=O)CN4CC(c5ccc(Nc6cnc7c(c6)CNC7)cc5C(F)(F)F)C4)ccc3C2=O)C(=O)N1] | 634.2, 317.7 |
| 8-9 | 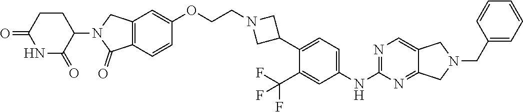 [SMILES: O=C1CCC(N2Cc3cc(OCCN4CC(c5ccc(Nc6ncc7c(n6)CN(Cc6ccccc6)C7)cc5C(F)(F)F)C4)ccc3C2=O)C(=O)N1] | 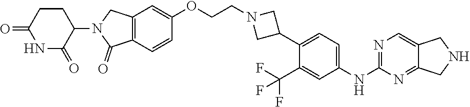 [SMILES: O=C1CCC(N2Cc3cc(OCCN4CC(c5ccc(Nc6ncc7c(n6)CNC7)cc5C(F)(F)F)C4)ccc3C2=O)C(=O)N1] | 622.4, 311.5 |
| 8-10 | 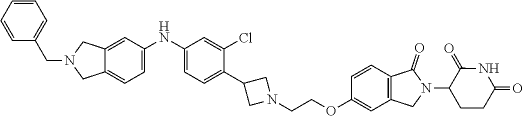 [SMILES: O=C1CCC(N2Cc3cc(OCCN4CC(c5ccc(Nc6ccc7c(c6)CN(Cc6ccccc6)C7)cc5Cl)C4)ccc3C2=O)C(=O)N1] | 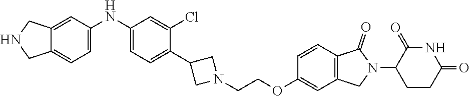 [SMILES: O=C1CCC(N2Cc3cc(OCCN4CC(c5ccc(Nc6ccc7c(c6)CNC7)cc5Cl)C4)ccc3C2=O)C(=O)N1] | 586.3, 293.6 |
| 8-11 | 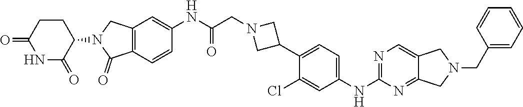 [SMILES: O=C1CC[C@H](N2Cc3cc(NC(=O)CN4CC(c5ccc(Nc6ncc7c(n6)CN(Cc6ccccc6)C7)cc5Cl)C4)ccc3C2=O)C(=O)N1] | 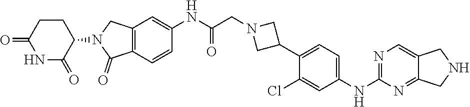 [SMILES: O=C1CC[C@H](N2Cc3cc(NC(=O)CN4CC(c5ccc(Nc6ncc7c(n6)CNC7)cc5Cl)C4)ccc3C2=O)C(=O)N1] | 601.1, 301.1 |
| 8-12 | 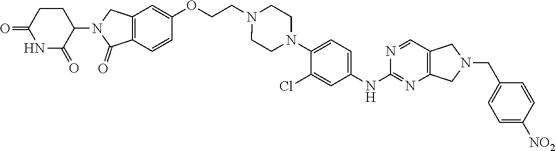 [SMILES: O=C1CCC(N2Cc3cc(OCCN4CCN(c5ccc(Nc6ncc7c(n6)CN(Cc6ccc([N+](=O)[O-])cc6)C7)cc5Cl)CC4)ccc3C2=O)C(=O)N1] | 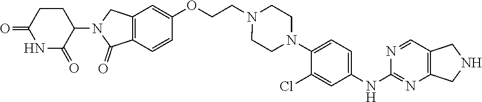 [SMILES: O=C1CCC(N2Cc3cc(OCCN4CCN(c5ccc(Nc6ncc7c(n6)CNC7)cc5Cl)CC4)ccc3C2=O)C(=O)N1] | 617.3, 619.3 |
| 8-13 | 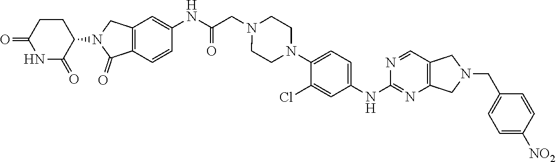 [SMILES: O=C1CC[C@H](N2Cc3cc(NC(=O)CN4CCN(c5ccc(Nc6ncc7c(n6)CN(Cc6ccc([N+](=O)[O-])cc6)C7)cc5Cl)CC4)ccc3C2=O)C(=O)N1] | 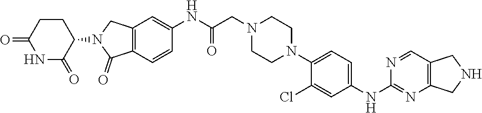 [SMILES: O=C1CC[C@H](N2Cc3cc(NC(=O)CN4CCN(c5ccc(Nc6ncc7c(n6)CNC7)cc5Cl)CC4)ccc3C2=O)C(=O)N1] | 630.4, 632.4 |
| 8-14 | 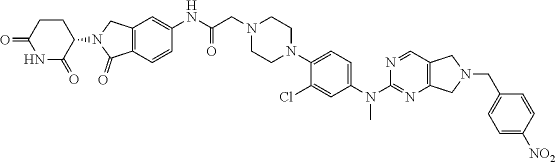 [SMILES: CN(c1ccc(N2CCN(CC(=O)Nc3ccc4c(c3)CN([C@H]3CCC(=O)NC3=O)C4=O)CC2)c(Cl)c1)c1ncc2c(n1)CN(Cc1ccc([N+](=O)[O-])cc1)C2] | 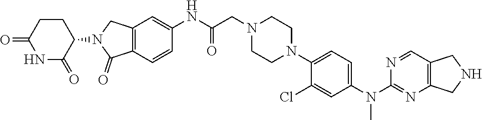 [SMILES: CN(c1ccc(N2CCN(CC(=O)Nc3ccc4c(c3)CN([C@H]3CCC(=O)NC3=O)C4=O)CC2)c(Cl)c1)c1ncc2c(n1)CNC2] | 644.4, 646.4 |

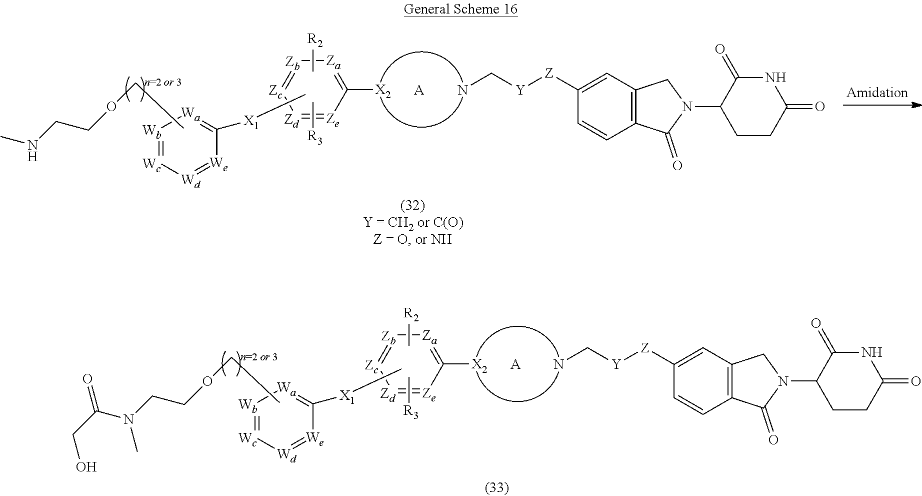

### Method 62

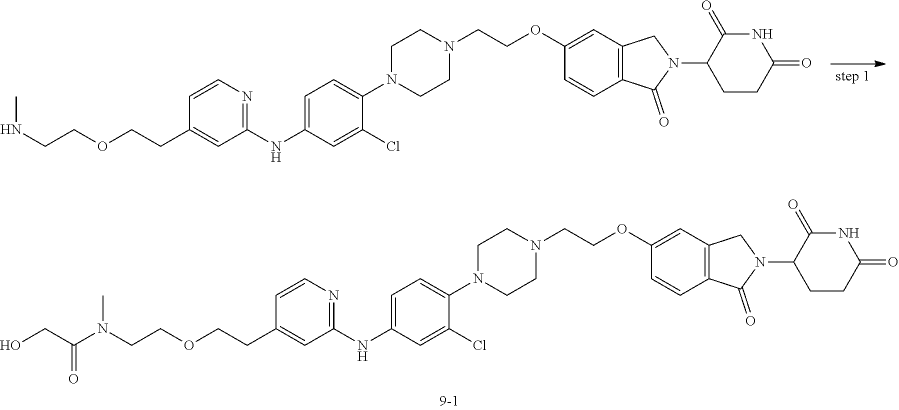 [SMILES: CNCCOCCc1ccnc(Nc2ccc(N3CCN(CCOc4ccc5c(c4)CN(C4CCC(=O)NC4=O)C5=O)CC3)c(Cl)c2)c1 ; CN(CCOCCc1ccnc(Nc2ccc(N3CCN(CCOc4ccc5c(c4)CN(C4CCC(=O)NC4=O)C5=O)CC3)c(Cl)c2)c1)C(=O)CO]

Step 1: A mixture of 3-[5-[2-[4-[2-chloro-4-[[4-[2-[2-(methylamino)ethoxy]ethyl]-2-pyridyl]amino]phenyl]piperazin-1-yl]ethoxy]-1-oxo-isoindolin-2-yl]piperidine-2,6-dione (11 mg, 16.27 μmol), 2-hydroxyacetic acid (1.86 mg, 24.40 μmol, 1.46 μL), and HATU (7.4 mg, 19.52 μmol) in DMF (1 mL) was treated with DIPEA (4.2 mg, 32.53 μmol, 5.67 μL), then the mixture was shaken for 2 hours at room temperature. The mixture was diluted with EtOAc (˜10 mL), washed a brine (˜10 mL) twice, dried over sodium sulfate, filtered, concentrated, and purified by a flash column to afford 9-1. MeOH (0 to 30%) in EtOAc. ESI-MS: m/z=734.5 [M+1]⁺.

The following compounds 9-1 through 9-15 (33) shown in Table 9 were prepared by methods similar to those described for the preparation of 9-1, using the appropriate amine intermediate (32).

TABLE 9

| | | Product | |
|---|---|---|---|
| Compd | 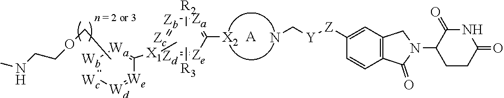 | 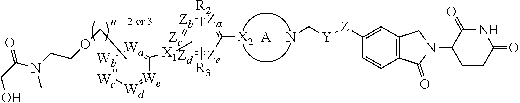 | MS found [M + H]+ |
| 9-1 | 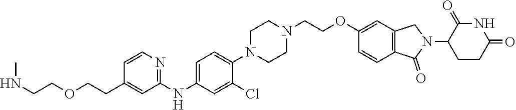 [SMILES: CNCCOCCc1ccnc(Nc2ccc(N3CCN(CCOc4ccc5c(c4)CN(C4CCC(=O)NC4=O)C5=O)CC3)c(Cl)c2)c1] | 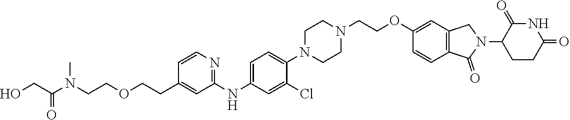 [SMILES: CN(CCOCCc1ccnc(Nc2ccc(N3CCN(CCOc4ccc5c(c4)CN(C4CCC(=O)NC4=O)C5=O)CC3)c(Cl)c2)c1)C(=O)CO] | 367.6, 734.5 |
| 9-2 | 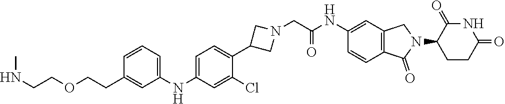 [SMILES: CNCCOCCc1cccc(Nc2ccc(C3CN(CC(=O)Nc4ccc5c(c4)CN([C@@H]4CCC(=O)NC4=O)C5=O)C3)c(Cl)c2)c1] | 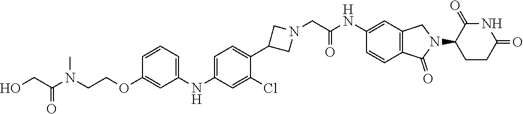 [SMILES: CN(CCOc1cccc(Nc2ccc(C3CN(CC(=O)Nc4ccc5c(c4)CN([C@@H]4CCC(=O)NC4=O)C5=O)C3)c(Cl)c2)c1)C(=O)CO] | 689.4 |
| 9-3 | 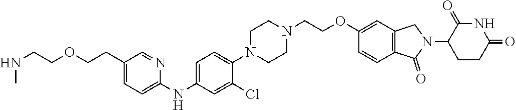 [SMILES: CNCCOCCc1ccc(Nc2ccc(N3CCN(CCOc4ccc5c(c4)CN(C4CCC(=O)NC4=O)C5=O)CC3)c(Cl)c2)nc1] | 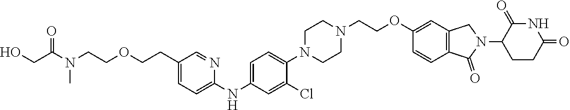 [SMILES: CN(CCOCCc1ccc(Nc2ccc(N3CCN(CCOc4ccc5c(c4)CN(C4CCC(=O)NC4=O)C5=O)CC3)c(Cl)c2)nc1)C(=O)CO] | 734.4, 367.8 |
| 9-4 | 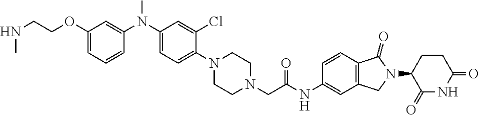 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 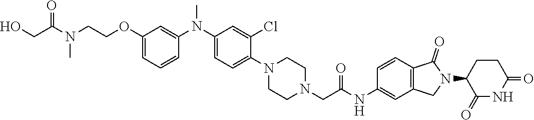 [SMILES: CN(CCOc1cccc(N(C)c2ccc(N3CCN(CC(=O)Nc4ccc5c(c4)CN([C@H]4CCC(=O)NC4=O)C5=O)CC3)c(Cl)c2)c1)C(=O)CO] | 732.4, 366.7 |
| 9 .-5 | 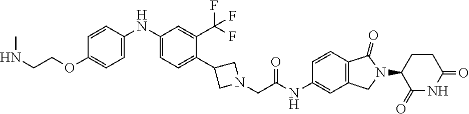 [SMILES: CNCCOc1ccc(Nc2ccc(C3CN(CC(=O)Nc4ccc5c(c4)CN([C@H]4CCC(=O)NC4=O)C5=O)C3)c(C(F)(F)F)c2)cc1] | 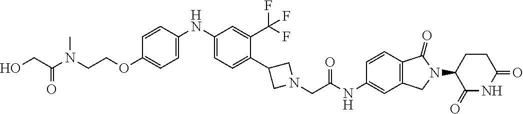 [SMILES: CN(CCOc1ccc(Nc2ccc(C3CN(CC(=O)Nc4ccc5c(c4)CN([C@H]4CCC(=O)NC4=O)C5=O)C3)c(C(F)(F)F)c2)cc1)C(=O)CO] | 723.3, 354.3 |
| 9-6 | 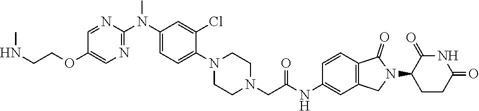 [SMILES: CNCCOc1cnc(N(C)c2ccc(N3CCN(CC(=O)Nc4ccc5c(c4)CN([C@@H]4CCC(=O)NC4=O)C5=O)CC3)c(Cl)c2)nc1] | 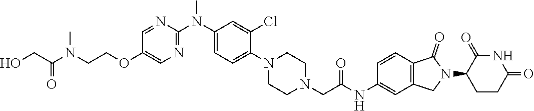 [SMILES: CN(CCOc1cnc(N(C)c2ccc(N3CCN(CC(=O)Nc4ccc5c(c4)CN([C@@H]4CCC(=O)NC4=O)C5=O)CC3)c(Cl)c2)nc1)C(=O)CO] | 367.7, 734.4 |
| 9-7 | 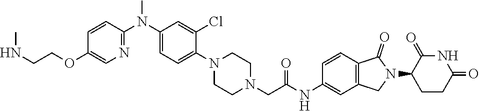 [SMILES: CNCCOc1ccc(N(C)c2ccc(N3CCN(CC(=O)Nc4ccc5c(c4)CN([C@@H]4CCC(=O)NC4=O)C5=O)CC3)c(Cl)c2)nc1] | 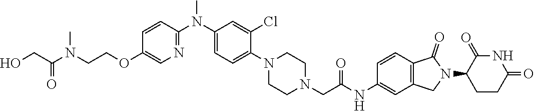 [SMILES: CN(CCOc1ccc(N(C)c2ccc(N3CCN(CC(=O)Nc4ccc5c(c4)CN([C@@H]4CCC(=O)NC4=O)C5=O)CC3)c(Cl)c2)nc1)C(=O)CO] | 733.4, 367.3 |
| 9-8 | 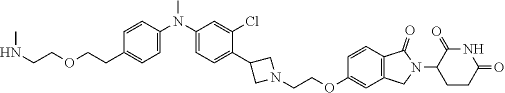 [SMILES: CNCCOCCc1ccc(N(C)c2ccc(C3CN(CCOc4ccc5c(c4)CN(C4CCC(=O)NC4=O)C5=O)C3)c(Cl)c2)cc1] | 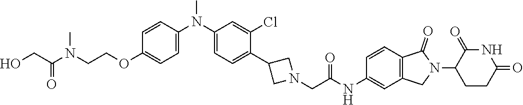 [SMILES: CN(CCOc1ccc(N(C)c2ccc(C3CN(CC(=O)Nc4ccc5c(c4)CN(C4CCC(=O)NC4=O)C5=O)C3)c(Cl)c2)cc1)C(=O)CO] | 359.8, 718.4 |
| 9-9 | 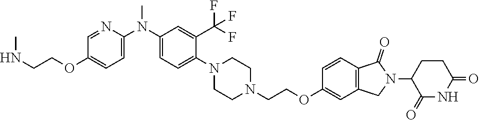 [SMILES: CNCCOc1ccc(N(C)c2ccc(N3CCN(CCOc4ccc5c(c4)CN(C4CCC(=O)NC4=O)C5=O)CC3)c(C(F)(F)F)c2)nc1] | 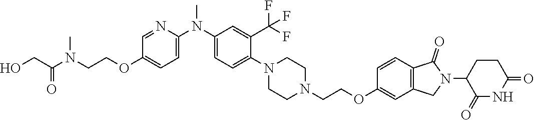 [SMILES: CN(CCOc1ccc(N(C)c2ccc(N3CCN(CCOc4ccc5c(c4)CN(C4CCC(=O)NC4=O)C5=O)CC3)c(C(F)(F)F)c2)nc1)C(=O)CO] | 754.3, 377.8 |
| 9-10 | 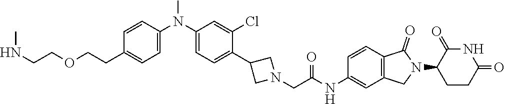 [SMILES: CNCCOCCc1ccc(N(C)c2ccc(C3CN(CC(=O)Nc4ccc5c(c4)CN([C@@H]4CCC(=O)NC4=O)C5=O)C3)c(Cl)c2)cc1] | 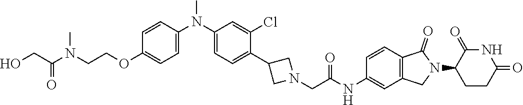 [SMILES: CN(CCOc1ccc(N(C)c2ccc(C3CN(CC(=O)Nc4ccc5c(c4)CN([C@@H]4CCC(=O)NC4=O)C5=O)C3)c(Cl)c2)cc1)C(=O)CO] | 366.4, 733.5 |
| 9-11 | 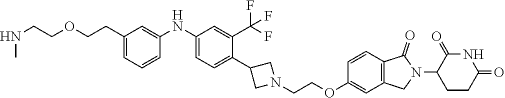 [SMILES: CNCCOCCc1cccc(Nc2ccc(C3CN(CCOc4ccc5c(c4)CN(C4CCC(=O)NC4=O)C5=O)C3)c(C(F)(F)F)c2)c1] | 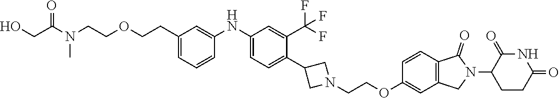 [SMILES: CN(CCOCCc1cccc(Nc2ccc(C3CN(CCOc4ccc5c(c4)CN(C4CCC(=O)NC4=O)C5=O)C3)c(C(F)(F)F)c2)c1)C(=O)CO] | 738.4, 369.7 |
| 9-12 | 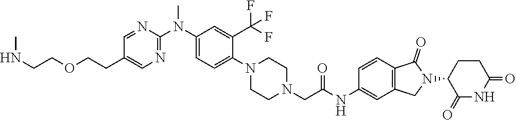 [SMILES: CNCCOCCc1cnc(N(C)c2ccc(N3CCN(CC(=O)Nc4ccc5c(c4)CN([C@@H]4CCC(=O)NC4=O)C5=O)CC3)c(C(F)(F)F)c2)nc1] | 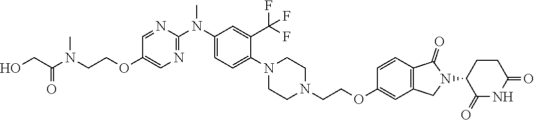 [SMILES: CN(CCOc1cnc(N(C)c2ccc(N3CCN(CCOc4ccc5c(c4)CN([C@@H]4CCC(=O)NC4=O)C5=O)CC3)c(C(F)(F)F)c2)nc1)C(=O)CO] | 796.5, 398.7 |
| 9-13 | 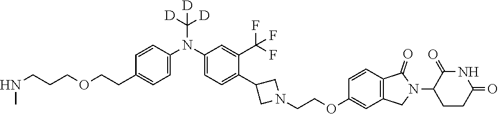 [SMILES: [2H]C([2H])([2H])N(c1ccc(CCOCCCNC)cc1)c1ccc(C2CN(CCOc3ccc4c(c3)CN(C3CCC(=O)NC3=O)C4=O)C2)c(C(F)(F)F)c1] | 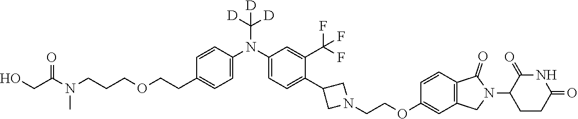 [SMILES: [2H]C([2H])([2H])N(c1ccc(CCOCCCN(C)C(=O)CO)cc1)c1ccc(C2CN(CCOc3ccc4c(c3)CN(C3CCC(=O)NC3=O)C4=O)C2)c(C(F)(F)F)c1] | 769.4, 385.3 |
| 9-14 | 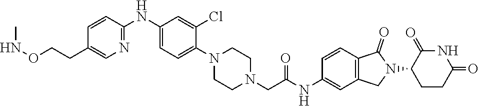 [SMILES: CNOCCc1ccc(Nc2ccc(N3CCN(CC(=O)Nc4ccc5c(c4)CN([C@H]4CCC(=O)NC4=O)C5=O)CC3)c(Cl)c2)nc1] | 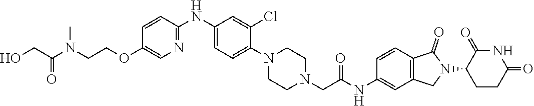 [SMILES: CN(CCOc1ccc(Nc2ccc(N3CCN(CC(=O)Nc4ccc5c(c4)CN([C@H]4CCC(=O)NC4=O)C5=O)CC3)c(Cl)c2)nc1)C(=O)CO] | 719.4, 721.4 |
| 9-15 | 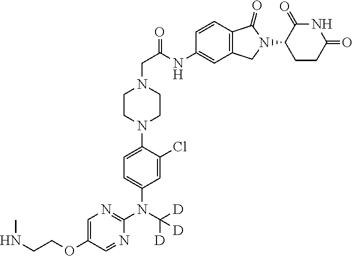 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCNC)cn1] | 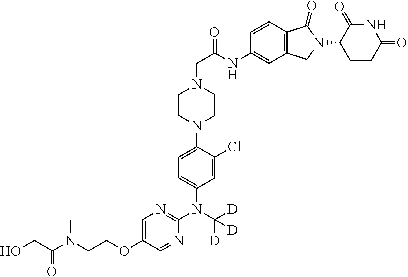 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCN(C)C(=O)CO)cn1] | 737.3 |

### Method 63

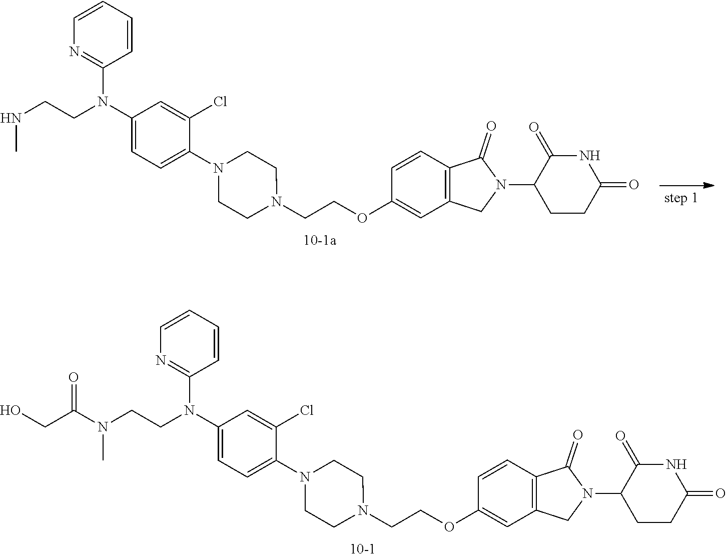 [SMILES: CNCCN(c1ccc(N2CCN(CCOc3ccc4c(c3)CN(C3CCC(=O)NC3=O)C4=O)CC2)c(Cl)c1)c1ccccn1 ; CN(CCN(c1ccc(N2CCN(CCOc3ccc4c(c3)CN(C3CCC(=O)NC3=O)C4=O)CC2)c(Cl)c1)c1ccccn1)C(=O)CO]

A mixture of 3-[5-[2-[4-[2-chloro-4-[2-(methylamino)ethyl-(2-pyridyl)amino]phenyl]piperazin-1-yl]ethoxy]-1-oxo-isoindolin-2-yl]piperidine-2,6-dione (36 mg, 56.95 μmol), 2-hydroxyacetic acid (8.66 mg, 113.90 μmol, 6.82 μL), and HATU (32 mg, 85.42 μmol) in DMF (3 mL) was treated with TEA (14 mg, 142.37 μmol, 19.84 μL), then the mixture was shaken for 2 hours at room temperature. The mixture was diluted with EtOAc (˜10 mL), washed a brine (˜10 mL) twice, dried over sodium sulfate, filtered, concentrated, and purified by a flash column to afford 10-1. MeOH (0 to 30%) in EtOAc. ESI-MS: m/z=690.4 [M+1]⁺.

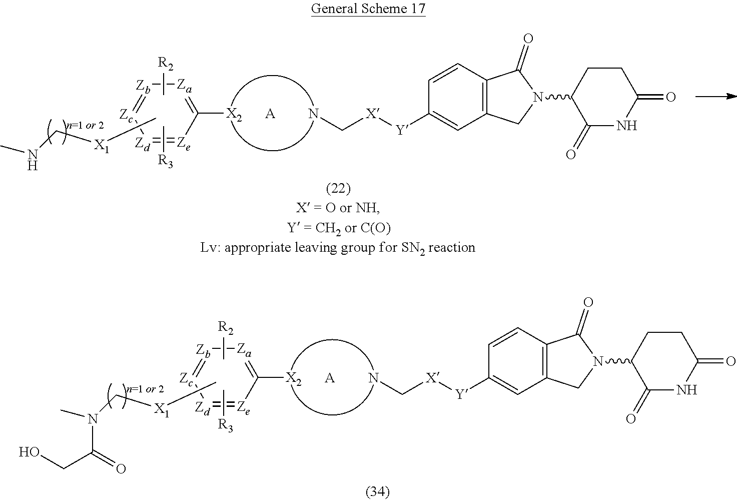

### Method 64

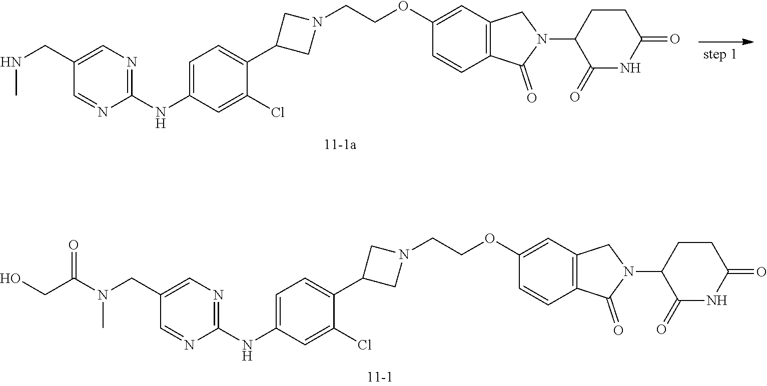 [SMILES: CNCc1cnc(Nc2ccc(C3CN(CCOc4ccc5c(c4)CN(C4CCC(=O)NC4=O)C5=O)C3)c(Cl)c2)nc1 ; CN(Cc1cnc(Nc2ccc(C3CN(CCOc4ccc5c(c4)CN(C4CCC(=O)NC4=O)C5=O)C3)c(Cl)c2)nc1)C(=O)CO]

A mixture of 3-[5-[2-[4-[2-chloro-4-[2-(methylamino)ethyl-(2-pyridyl)amino]phenyl]piperazin-1-yl]ethoxy]-1-oxo-isoindolin-2-yl]piperidine-2,6-dione (36 mg, 56.95 μmol), 2-hydroxyacetic acid (8.66 mg, 113.90 μmol, 6.82 μL), and HATU (32 mg, 85.42 μmol) in DMF (3 mL) was treated with TEA (14 mg, 142.37 μmol, 19.84 μL), then the mixture was shaken for 2 hours at room temperature. The mixture was diluted with EtOAc (˜10 mL), washed brine (˜10 mL) twice, dried over sodium sulfate, filtered, concentrated, and purified by a flash column to afford 11-1. MeOH (0 to 300%) in EtOAc. ESI-MS: m/z=690.4, [M+1]⁺.

The following compounds 11-1 through 11-4 (34) shown in Table 10 were prepared by methods similar to those described for the preparation of 11-1, using the appropriate amine intermediate (22).

TABLE 10

| | | Product | |
|---|---|---|---|
| Compd | 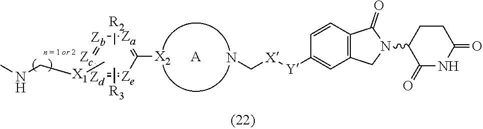 | 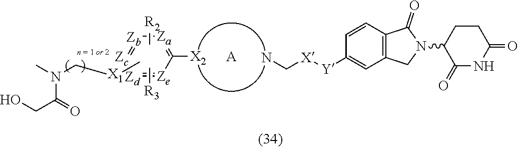 | MS found [M + H]+ |
| 11-1 | 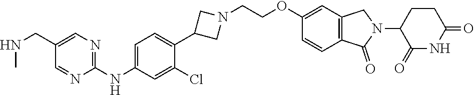 [SMILES: CNCc1cnc(Nc2ccc(C3CN(CCOc4ccc5c(c4)CN(C4CCC(=O)NC4=O)C5=O)C3)c(Cl)c2)nc1] | 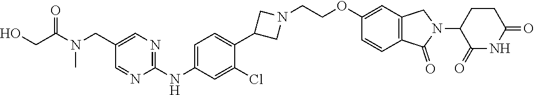 [SMILES: CN(Cc1cnc(Nc2ccc(C3CN(CCOc4ccc5c(c4)CN(C4CCC(=O)NC4=O)C5=O)C3)c(Cl)c2)nc1)C(=O)CO] | 648.3 |
| 11-2 | 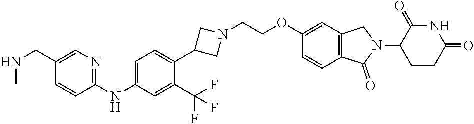 [SMILES: CNCc1ccc(Nc2ccc(C3CN(CCOc4ccc5c(c4)CN(C4CCC(=O)NC4=O)C5=O)C3)c(C(F)(F)F)c2)nc1] | 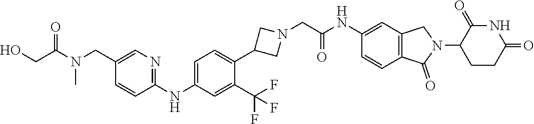 [SMILES: CN(Cc1ccc(Nc2ccc(C3CN(CC(=O)Nc4ccc5c(c4)CN(C4CCC(=O)NC4=O)C5=O)C3)c(C(F)(F)F)c2)nc1)C(=O)CO] | 694.3, 347.8 |
| 11-3 | 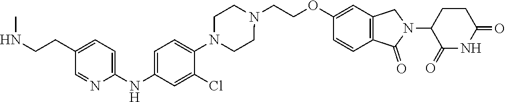 [SMILES: CNCCc1ccc(Nc2ccc(N3CCN(CCOc4ccc5c(c4)CN(C4CCC(=O)NC4=O)C5=O)CC3)c(Cl)c2)nc1] | 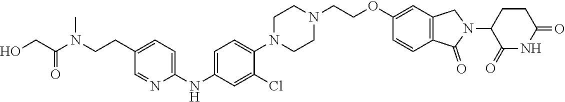 [SMILES: CN(CCc1ccc(Nc2ccc(N3CCN(CCOc4ccc5c(c4)CN(C4CCC(=O)NC4=O)C5=O)CC3)c(Cl)c2)nc1)C(=O)CO] | 690.4, 345.8 |
| 11-4 | 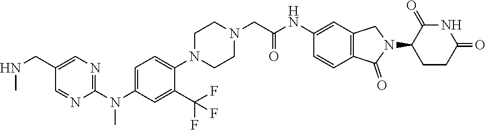 [SMILES: CNCc1cnc(N(C)c2ccc(N3CCN(CC(=O)Nc4ccc5c(c4)CN([C@@H]4CCC(=O)NC4=O)C5=O)CC3)c(C(F)(F)F)c2)nc1] | 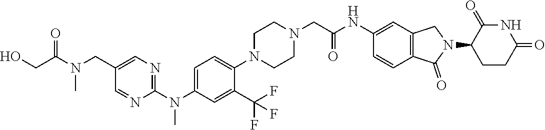 [SMILES: CN(Cc1cnc(N(C)c2ccc(N3CCN(CC(=O)Nc4ccc5c(c4)CN([C@@H]4CCC(=O)NC4=O)C5=O)CC3)c(C(F)(F)F)c2)nc1)C(=O)CO] | 369.9, 738.4 |

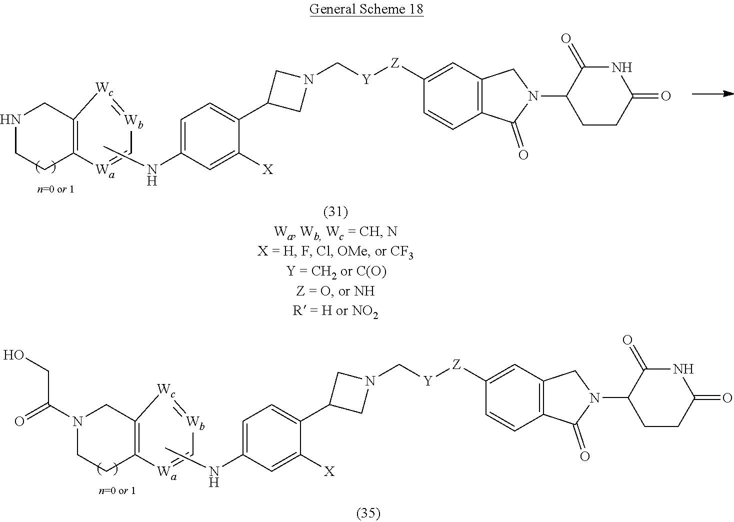

### Method 65

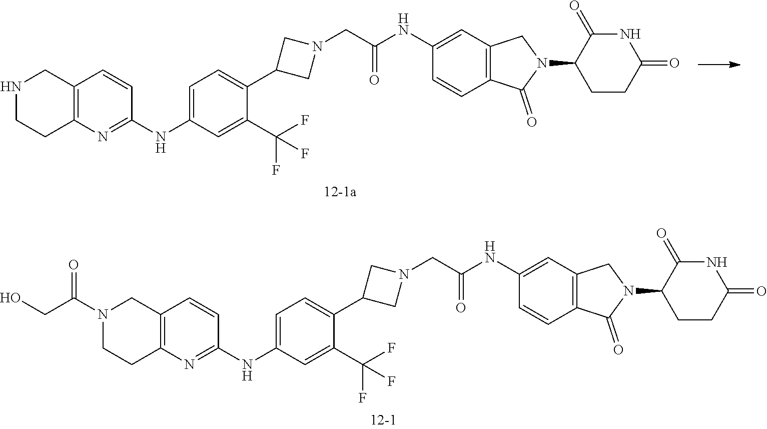 [SMILES: O=C1CC[C@@H](N2Cc3cc(NC(=O)CN4CC(c5ccc(Nc6ccc7c(n6)CCNC7)cc5C(F)(F)F)C4)ccc3C2=O)C(=O)N1 ; O=C1CC[C@@H](N2Cc3cc(NC(=O)CN4CC(c5ccc(Nc6ccc7c(n6)CCN(C(=O)CO)C7)cc5C(F)(F)F)C4)ccc3C2=O)C(=O)N1]

A mixture of 3-[5-[2-[3-[4-[(6-benzyl-7,8-dihydro-5H-1,6-naphthyridin-2-yl)amino]-2-(trifluoromethyl)phenyl]azetidin-1-yl]ethoxy]-1-oxo-isoindolin-2-yl]piperidine-2,6-dione (30 mg, 39.41 μmol, CL) and Palladium, 10% on carbon, Type 487, dry (419.40 μg, 3.94 μmol) in MeOH (5 mL) was equipped hydrogen gas ballon and stirred for 1 hour at a room temperature. The mixture was filtered through a celite filter, and the filtrate was collected, concentrated, and purified by a flash column to 12-1. MeOH 0 to 40% gradient in EtOAc. ESI-MS: m/z=676.3 [M+1]⁺.

The following compounds 12-1 through 12-4 (35) shown in Table 11 were prepared by methods similar to those described for the preparation of 12-1, using the appropriate amine intermediate (31).

TABLE 11

| Compd | 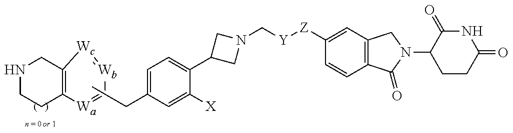 (31) | Product 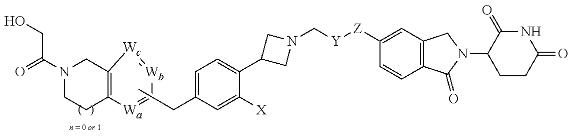 (35) | MS found [M + H]+ |
|---|---|---|---|
| 12-1 | 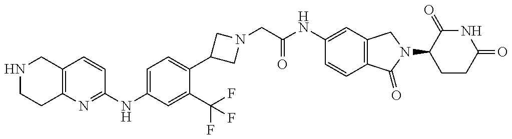 [SMILES: O=C1CC[C@@H](N2Cc3cc(NC(=O)CN4CC(c5ccc(Nc6ccc7c(n6)CCNC7)cc5C(F)(F)F)C4)ccc3C2=O)C(=O)N1] | 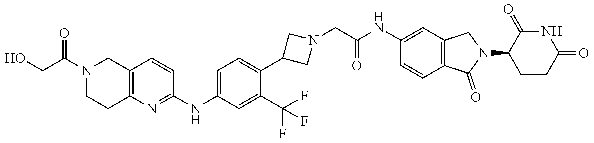 [SMILES: O=C1CC[C@@H](N2Cc3cc(NC(=O)CN4CC(c5ccc(Nc6ccc7c(n6)CCN(C(=O)CO)C7)cc5C(F)(F)F)C4)ccc3C2=O)C(=O)N1] | 706.5, 353.7 |
| 12-2 | 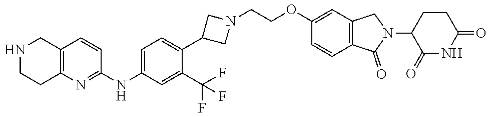 [SMILES: O=C1CCC(N2Cc3cc(OCCN4CC(c5ccc(Nc6ccc7c(n6)CCNC7)cc5C(F)(F)F)C4)ccc3C2=O)C(=O)N1] | 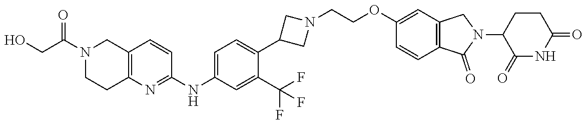 [SMILES: O=C1CCC(N2Cc3cc(OCCN4CC(c5ccc(Nc6ccc7c(n6)CCN(C(=O)CO)C7)cc5C(F)(F)F)C4)ccc3C2=O)C(=O)N1] | 693.4, 346.9 |
| 12-3 | 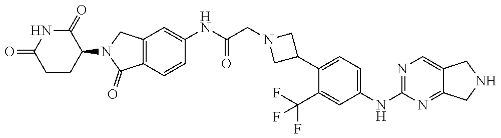 [SMILES: O=C1CC[C@H](N2Cc3cc(NC(=O)CN4CC(c5ccc(Nc6ncc7c(n6)CNC7)cc5C(F)(F)F)C4)ccc3C2=O)C(=O)N1] | 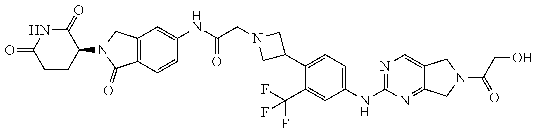 [SMILES: O=C1CC[C@H](N2Cc3cc(NC(=O)CN4CC(c5ccc(Nc6ncc7c(n6)CN(C(=O)CO)C7)cc5C(F)(F)F)C4)ccc3C2=O)C(=O)N1] | 693.4, 347.3 |
| 12-4 | 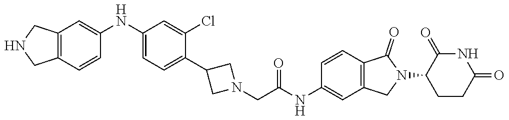 [SMILES: O=C1CC[C@H](N2Cc3cc(NC(=O)CN4CC(c5ccc(Nc6ccc7c(c6)CNC7)cc5Cl)C4)ccc3C2=O)C(=O)N1] | 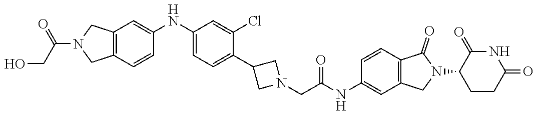 [SMILES: O=C1CC[C@H](N2Cc3cc(NC(=O)CN4CC(c5ccc(Nc6ccc7c(c6)CN(C(=O)CO)C7)cc5Cl)C4)ccc3C2=O)C(=O)N1] | 657.3 |

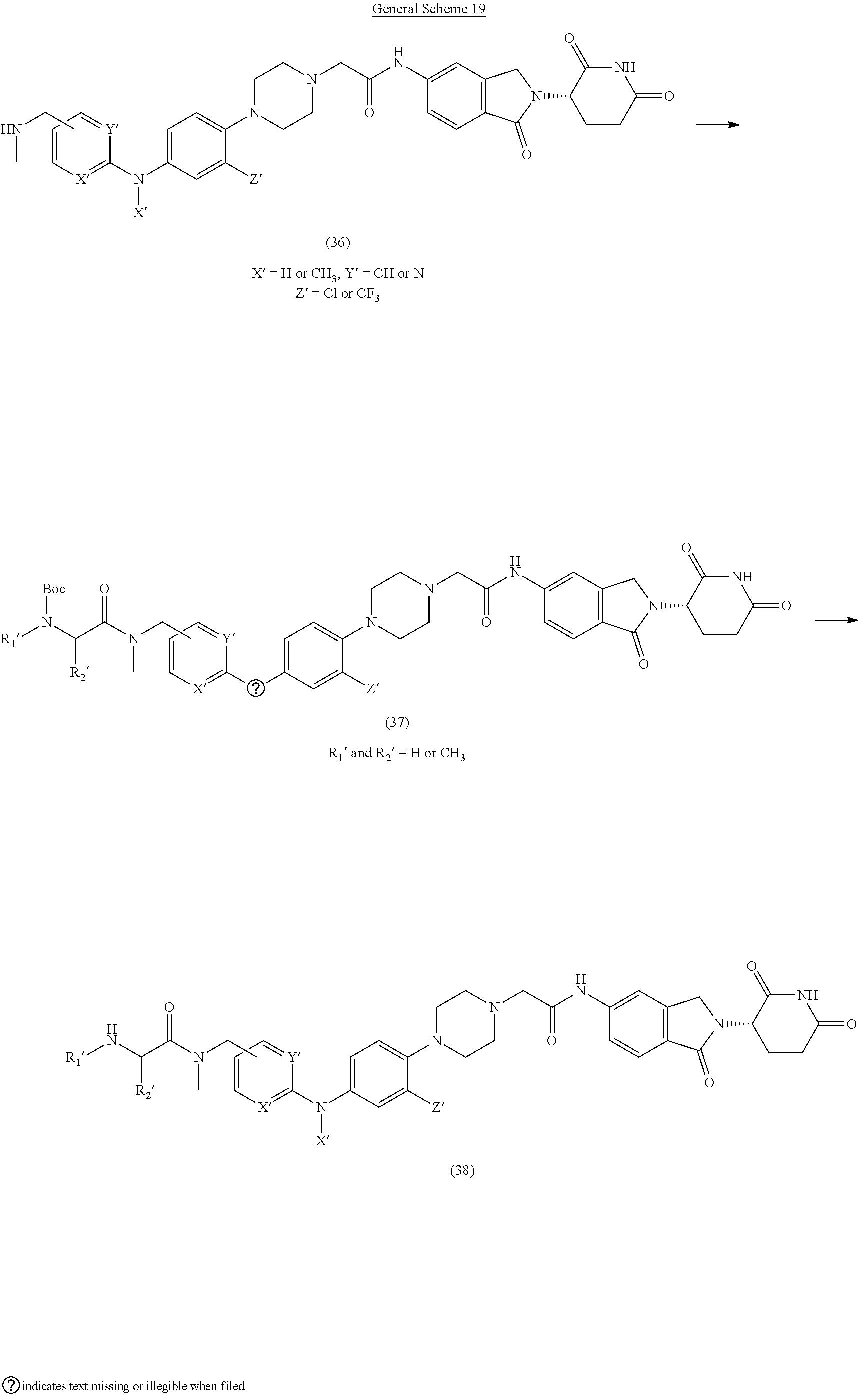

### Method 66

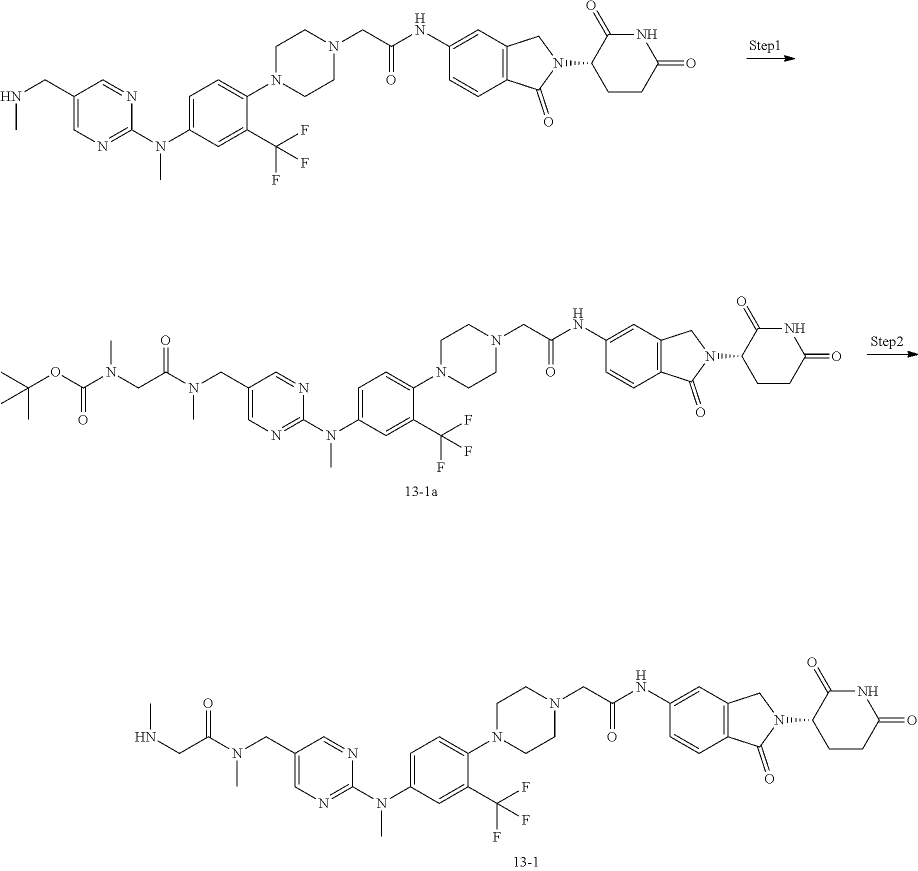 [SMILES: CNCc1cnc(N(C)c2ccc(N3CCN(CC(=O)Nc4ccc5c(c4)CN([C@H]4CCC(=O)NC4=O)C5=O)CC3)c(C(F)(F)F)c2)nc1 ; CN(Cc1cnc(N(C)c2ccc(N3CCN(CC(=O)Nc4ccc5c(c4)CN([C@H]4CCC(=O)NC4=O)C5=O)CC3)c(C(F)(F)F)c2)nc1)C(=O)CN(C)C(=O)OC(C)(C)C ; CNCC(=O)N(C)Cc1cnc(N(C)c2ccc(N3CCN(CC(=O)Nc4ccc5c(c4)CN([C@H]4CCC(=O)NC4=O)C5=O)CC3)c(C(F)(F)F)c2)nc1]

Step 1: A mixture of N-[2-[(3S)-2,6-dioxo-3-piperidyl]-1-oxo-isoindolin-5-yl]-2-[4-[4-[methyl-[5-(methylaminomethyl)pyrimidin-2-yl]amino]-2-(trifluoromethyl)phenyl]piperazin-1-yl]acetamide (50 mg, 73.56 μmol), 2-[tert-butoxycarbonyl(methyl)amino]acetic acid (27.84 mg, 147.13 μmol), and HATU (40 mg, 110.34 μmol) in DMF (2 mL) was treated with TEA (30 mg, 257.47 μmol, 35.89 μL), then the mixture was shaken for 2 hours at room temperature. The mixture was diluted with EtOAc (˜10 mL), washed a brine (˜10 mL) twice, dried over sodium sulfate, filtered, concentrated, and purified by a flash column. MeOH (0 to 30%) in EtOAc to afford 13-1a. ESI-MS: m/z=851.4 [M+1]⁺.
Step 2: A solution of tert-butyl N-[2-[[2-[4-[4-[2-[[2-[(3S)-2,6-dioxo-3-piperidyl]-1-oxo-isoindolin-5-yl]amino]-2-oxo-ethyl]piperazin-1-yl]-N-methyl-3-(trifluoromethyl)anilino]pyrimidin-5-yl]methyl-methyl-amino]-2-oxo-ethyl]-N-methyl-carbamate (50 mg, 58.76 μmol) in TFA (0.5 mL) and DCM (5 mL) was shaken for 1 hour at room temperature. The mixture was concentrated and purified by a flash column (MeOH+3% TEA (30 to 100% in DCM) to afford 13-1 (25 mg, 54%). ESI-MS: m/z=751.6 [M+1]⁺.

The following compounds 13-1 through 13-4 (38) shown in Table 12 were prepared by methods similar to those described for the preparation of 13-1, using the appropriate amine intermediate (36).

TABLE 12

| Comp | 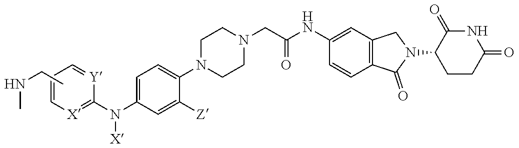 (36) | Product 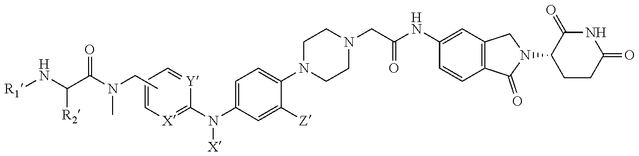 (38) | MS found [M + H]+ |
|---|---|---|---|
| 13- 1 | 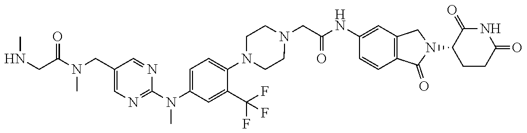 [SMILES: CNCC(=O)N(C)Cc1cnc(N(C)c2ccc(N3CCN(CC(=O)Nc4ccc5c(c4)CN([C@H]4CCC(=O)NC4=O)C5=O)CC3)c(C(F)(F)F)c2)nc1] | 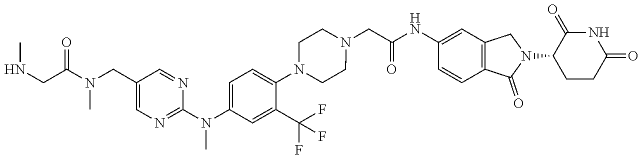 [SMILES: CNCC(=O)N(C)Cc1cnc(N(C)c2ccc(N3CCN(CC(=O)Nc4ccc5c(c4)CN([C@H]4CCC(=O)NC4=O)C5=O)CC3)c(C(F)(F)F)c2)nc1] | 376.2, 751.6 |
| 13- 2 | 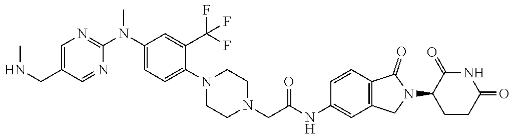 [SMILES: CNCc1cnc(N(C)c2ccc(N3CCN(CC(=O)Nc4ccc5c(c4)CN([C@@H]4CCC(=O)NC4=O)C5=O)CC3)c(C(F)(F)F)c2)nc1] | 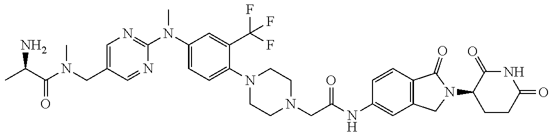 [SMILES: C[C@@H](N)C(=O)N(C)Cc1cnc(N(C)c2ccc(N3CCN(CC(=O)Nc4ccc5c(c4)CN([C@@H]4CCC(=O)NC4=O)C5=O)CC3)c(C(F)(F)F)c2)nc1] | 751.5, 376.5 |
| 13- 3 | 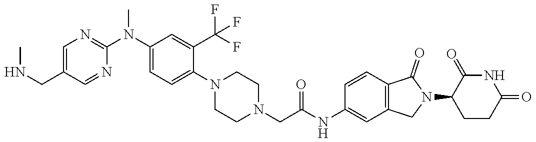 [SMILES: CNCc1cnc(N(C)c2ccc(N3CCN(CC(=O)Nc4ccc5c(c4)CN([C@@H]4CCC(=O)NC4=O)C5=O)CC3)c(C(F)(F)F)c2)nc1] | 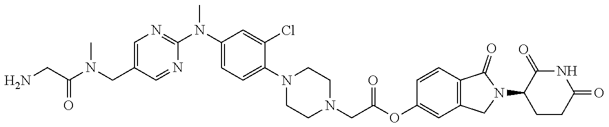 [SMILES: CN(Cc1cnc(N(C)c2ccc(N3CCN(CC(=O)Oc4ccc5c(c4)CN([C@@H]4CCC(=O)NC4=O)C5=O)CC3)c(Cl)c2)nc1)C(=O)CN] | 369.4, 737.4 |
| 13- 4 | 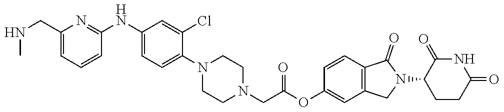 [SMILES: CNCc1cccc(Nc2ccc(N3CCN(CC(=O)Oc4ccc5c(c4)CN([C@H]4CCC(=O)NC4=O)C5=O)CC3)c(Cl)c2)n1] | 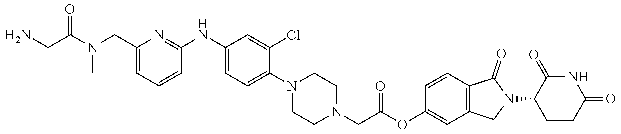 [SMILES: CN(Cc1cccc(Nc2ccc(N3CCN(CC(=O)Oc4ccc5c(c4)CN([C@H]4CCC(=O)NC4=O)C5=O)CC3)c(Cl)c2)n1)C(=O)CN] | 689.4, 691.5 |

### Method 67

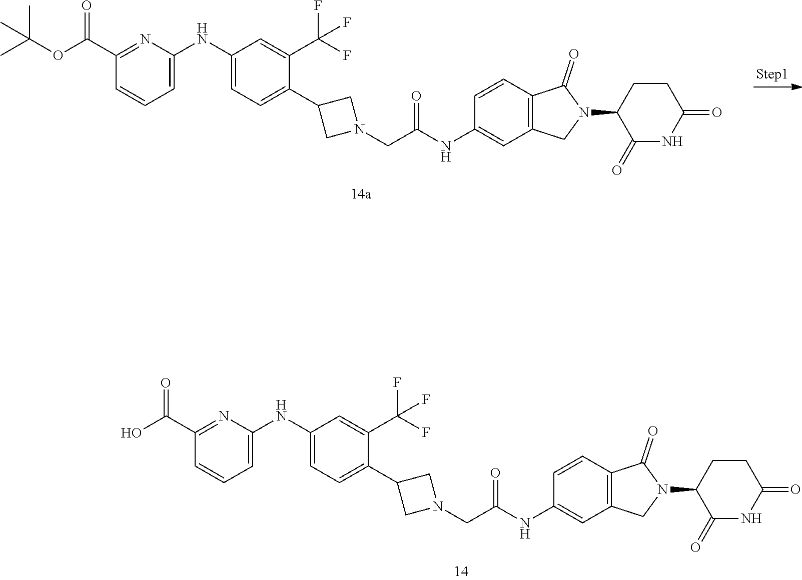 [SMILES: CC(C)(C)OC(=O)c1cccc(Nc2ccc(C3CN(CC(=O)Nc4ccc5c(c4)CN([C@H]4CCC(=O)NC4=O)C5=O)C3)c(C(F)(F)F)c2)n1 ; O=C1CC[C@H](N2Cc3cc(NC(=O)CN4CC(c5ccc(Nc6cccc(C(=O)O)n6)cc5C(F)(F)F)C4)ccc3C2=O)C(=O)N1]

A solution of tert-butyl 6-[4-[1-[2-[[2-[(3S)-2,6-dioxo-3-piperidyl]-1-oxo-isoindolin-5-yl]amino]-2-oxo-ethyl]azetidin-3-yl]-3-(trifluoromethyl)anilino]pyridine-2-carboxylate (10 mg, 14.44 μmol) in DCM (0.5 mL) and TFA (0.5 mL) was shaken for 4 hours at room temperature. The mixture was concentrated and purified by a flash column (MeOH+3% TEA (30 to 100% in DCM) to afford 14 (9 mg, 90%). ESI-MS: m/z=637.2 [M+1]⁺.

### Method 68

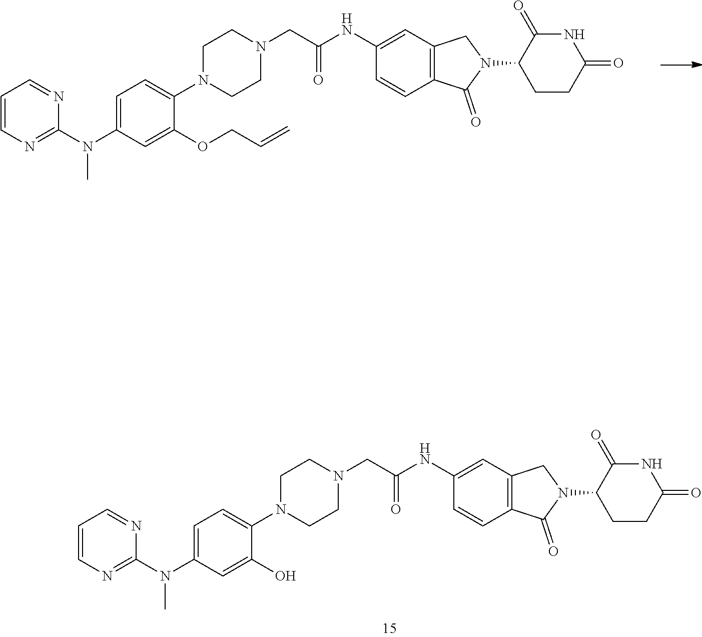 [SMILES: C=CCOc1cc(N(C)c2ncccn2)ccc1N1CCN(CC(=O)Nc2ccc3c(c2)CN([C@H]2CCC(=O)NC2=O)C3=O)CC1 ; CN(c1ccc(N2CCN(CC(=O)Nc3ccc4c(c3)CN([C@H]3CCC(=O)NC3=O)C4=O)CC2)c(O)c1)c1ncccn1]

To a solution of (S)-2-(4-(2-(allyloxy)-4-(methyl(pyrimidin-2-yl) amino) phenyl) piperazin-1-yl)-N-(2-(2,6-dioxopiperidin-3-yl)-1-oxoisoindolin-5-yl)s acetamide (120 mg, 0.19 mmol) and Pd(PPh₃)₄ (240 mg) in DCE (2 mL) stirred under nitrogen was added 1,3-Dimethylbarbituric acid (243 mg, 1.54 mmol). The reaction was stirred at 50° C. for 16 hours. The reaction mixture was poured into water (10 mL) and extracted with DCM (10 mL×3). The combined organic layers were washed with brine, dried over Na₂SO₄ and concentrated to afford a crude product. The residue was purified by prep-HPLC and lyophilized to give (S)—N-(2-(2,6-dioxopiperidin-3-yl)-1-oxoisoindolin-5-yl)-2-(4-(2-hydroxy-4-(methyl(pyrimidin-2-yl) amino) phenyl) piperazin-1-yl) acetamide (15, 20 mg, 17.76% yield) as a white solid. ESI-MS: m/z=585.3 [M+1]⁺, 293.3 [M/2+1]⁺. ¹H NMR (400 MHz, DMSO_d₆) δ 1.95-2.05 (m, 1H), 2.34-2.43 (m, 1H), 2.53-2.66 (m, 1H), 2.71 (s, 4H), 2.84-3.11 (m, 5H), 3.25 (s, 2H), 3.37 (s, 3H), 4.37 (dd, J=56.8, 17.4 Hz, 2H), 5.09 (dd, J=13.2, 5.0 Hz, 1H), 6.63-6.74 (m, 2H), 6.89 (d, J=8.4 Hz, 1H), 7.62-7.74 (m, 2H), 8.03 (s, 1H), 8.33 (d, J=4.8 Hz, 2H), 9.07 (s, 1H), 10.13 (s, 1H), 10.99 (s, 1H).

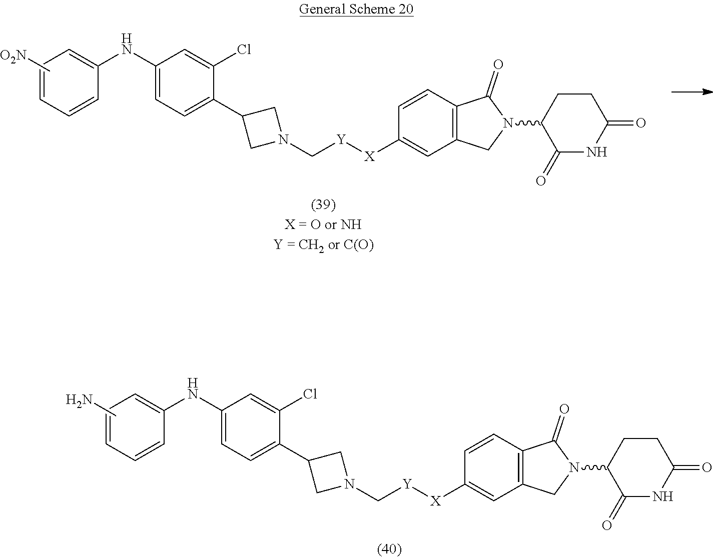

### Method 69

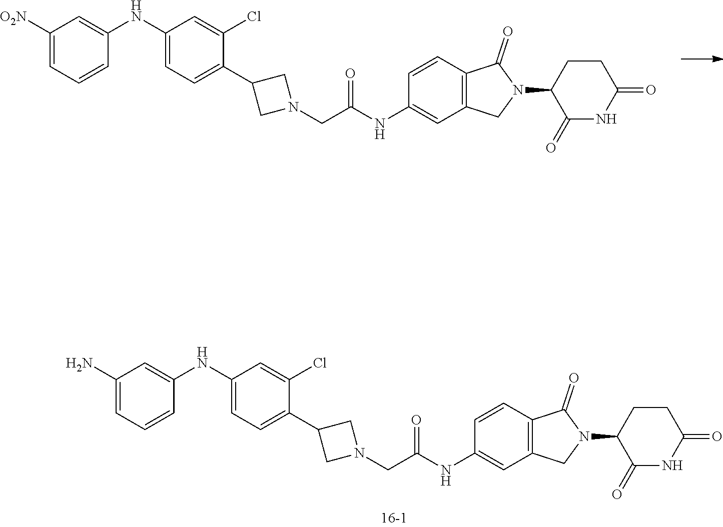 [SMILES: O=C1CC[C@H](N2Cc3cc(NC(=O)CN4CC(c5ccc(Nc6cccc([N+](=O)[O-])c6)cc5Cl)C4)ccc3C2=O)C(=O)N1 ; Nc1cccc(Nc2ccc(C3CN(CC(=O)Nc4ccc5c(c4)CN([C@H]4CCC(=O)NC4=O)C5=O)C3)c(Cl)c2)c1]

A solution of [3-[3-chloro-4-[1-[2-[[2-[(3S)-2,6-dioxo-3-piperidyl]-1-oxo-isoindolin-5-yl]amino]-2-oxo-ethyl]azetidin-3-yl]anilino]phenyl]azinate (62.99 mg, 104.28 μmol) in EtOH (2 mL) and THF (1 mL) was treated with dichlorotin;dihydrate (117.65 mg, 521.39 μmol), then the mixture was stirred for overnight at room temperature. The mixture was filtered via a cellite, then the filtrate was collected, concentrated, and purified by a flash column to afford 15 {2-[3-[4-(3-aminoanilino)-2-chloro-phenyl]azetidin-1-yl]-N-[2-[(3S)-2,6-dioxo-3-piperidyl]-1-oxo-isoindolin-5-yl]acetamide (16-1, 8 mg, 9.07 μmol, 8.70% yield, 65% purity)}. MeOH (0 to 30%) in EtOAc. ESI-MS: m/z=573.4 [M+1]⁺

The following compounds 16-1 through 16-7 (40) shown in Table 13 were prepared by methods similar to those described for the preparation of 16-1, using the appropriate nitro intermediate (39).

TABLE 13

| Compd | 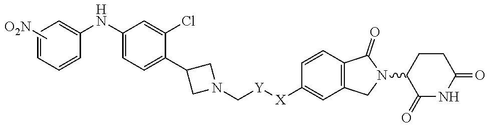 (39) | Product 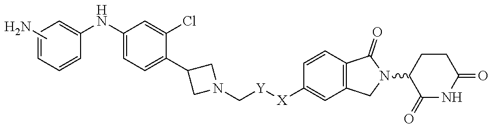 (40) | MS found [M + H]+ |
|---|---|---|---|
| 16-1 | 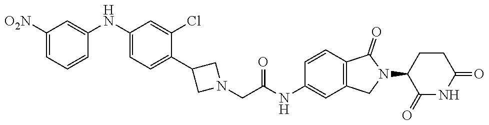 [SMILES: O=C1CC[C@H](N2Cc3cc(NC(=O)CN4CC(c5ccc(Nc6cccc([N+](=O)[O-])c6)cc5Cl)C4)ccc3C2=O)C(=O)N1] | 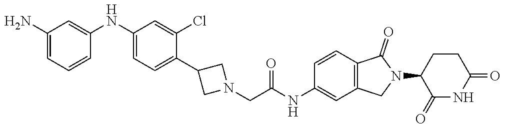 [SMILES: Nc1cccc(Nc2ccc(C3CN(CC(=O)Nc4ccc5c(c4)CN([C@H]4CCC(=O)NC4=O)C5=O)C3)c(Cl)c2)c1] | 573.4 |
| 16-2 | 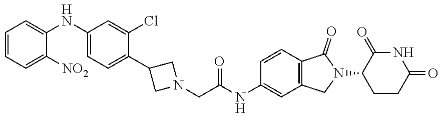 [SMILES: O=C1CC[C@H](N2Cc3cc(NC(=O)CN4CC(c5ccc(Nc6ccccc6[N+](=O)[O-])cc5Cl)C4)ccc3C2=O)C(=O)N1] | 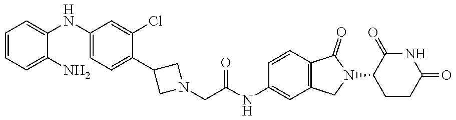 [SMILES: Nc1ccccc1Nc1ccc(C2CN(CC(=O)Nc3ccc4c(c3)CN([C@H]3CCC(=O)NC3=O)C4=O)C2)c(Cl)c1] | 573.4, 287.2 |
| 16-3 | 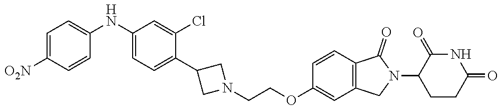 [SMILES: O=C1CCC(N2Cc3cc(OCCN4CC(c5ccc(Nc6ccc([N+](=O)[O-])cc6)cc5Cl)C4)ccc3C2=O)C(=O)N1] | 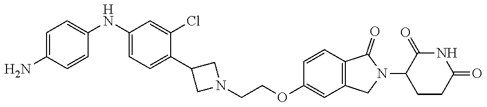 [SMILES: Nc1ccc(Nc2ccc(C3CN(CCOc4ccc5c(c4)CN(C4CCC(=O)NC4=O)C5=O)C3)c(Cl)c2)cc1] | 558.4, 279.7 |
| 16-4 | 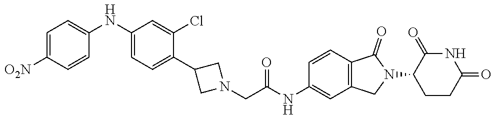 [SMILES: O=C1CC[C@H](N2Cc3cc(NC(=O)CN4CC(c5ccc(Nc6ccc([N+](=O)[O-])cc6)cc5Cl)C4)ccc3C2=O)C(=O)N1] | 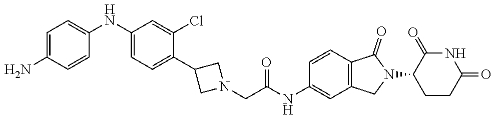 [SMILES: Nc1ccc(Nc2ccc(C3CN(CC(=O)Nc4ccc5c(c4)CN([C@H]4CCC(=O)NC4=O)C5=O)C3)c(Cl)c2)cc1] | 574.3, 287.3 |
| 16-5 | 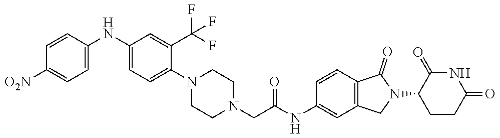 [SMILES: O=C1CC[C@H](N2Cc3cc(NC(=O)CN4CCN(c5ccc(Nc6ccc([N+](=O)[O-])cc6)cc5C(F)(F)F)CC4)ccc3C2=O)C(=O)N1] | 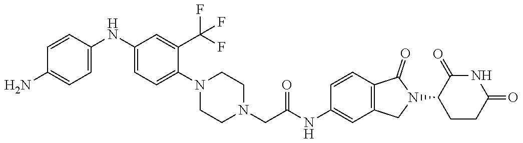 [SMILES: Nc1ccc(Nc2ccc(N3CCN(CC(=O)Nc4ccc5c(c4)CN([C@H]4CCC(=O)NC4=O)C5=O)CC3)c(C(F)(F)F)c2)cc1] | 636.4 |
| 16-6 | 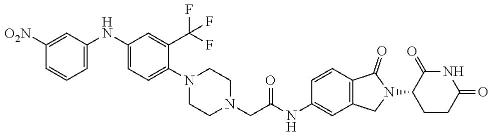 [SMILES: O=C1CC[C@H](N2Cc3cc(NC(=O)CN4CCN(c5ccc(Nc6cccc([N+](=O)[O-])c6)cc5C(F)(F)F)CC4)ccc3C2=O)C(=O)N1] | 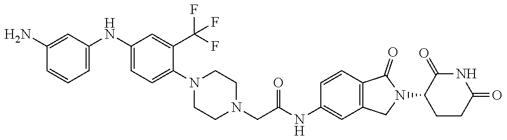 [SMILES: Nc1cccc(Nc2ccc(N3CCN(CC(=O)Nc4ccc5c(c4)CN([C@H]4CCC(=O)NC4=O)C5=O)CC3)c(C(F)(F)F)c2)c1] | 636.3 |
| 16-7 | 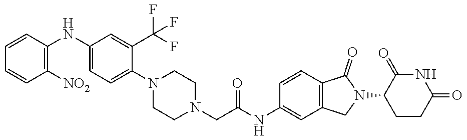 [SMILES: O=C1CC[C@H](N2Cc3cc(NC(=O)CN4CCN(c5ccc(Nc6ccccc6[N+](=O)[O-])cc5C(F)(F)F)CC4)ccc3C2=O)C(=O)N1] | 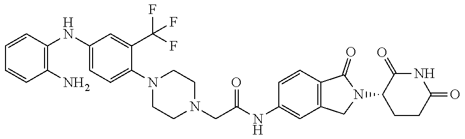 [SMILES: Nc1ccccc1Nc1ccc(N2CCN(CC(=O)Nc3ccc4c(c3)CN([C@H]3CCC(=O)NC3=O)C4=O)CC2)c(C(F)(F)F)c1] | 636.4 |

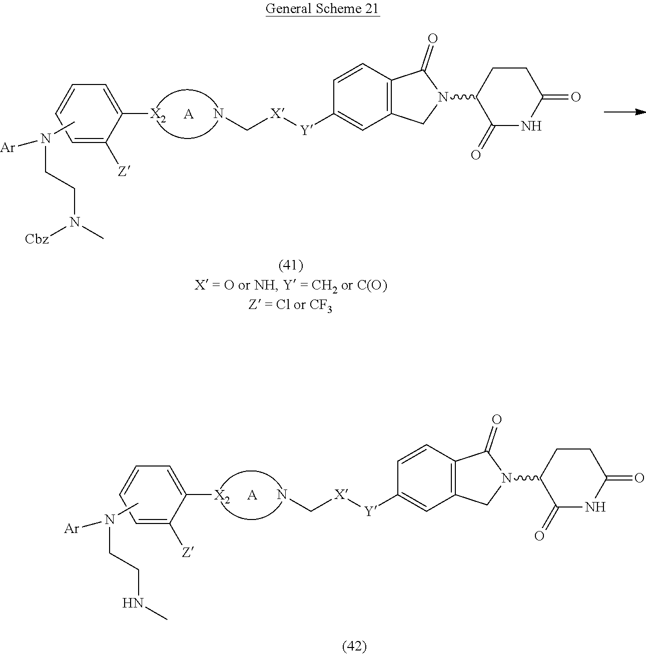

### Method 70

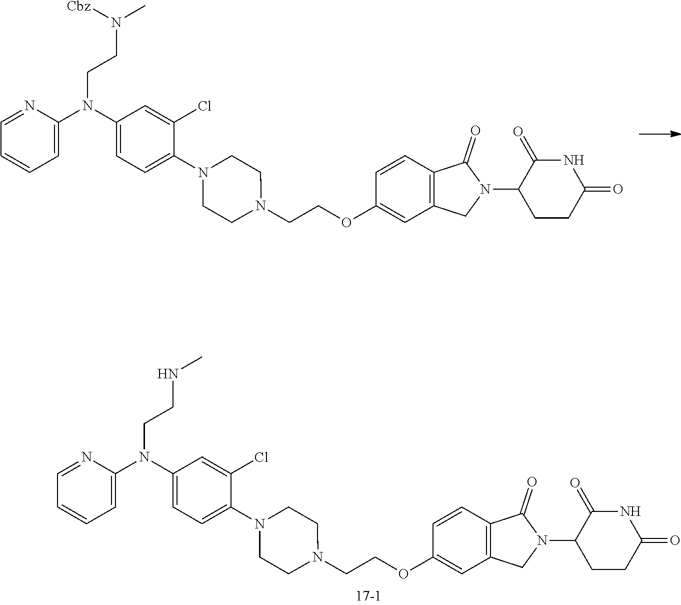 [SMILES: CN(CCN(c1ccc(N2CCN(CCOc3ccc4c(c3)CN(C3CCC(=O)NC3=O)C4=O)CC2)c(Cl)c1)c1ccccn1)C(=O)OCc1ccccc1 ; CNCCN(c1ccc(N2CCN(CCOc3ccc4c(c3)CN(C3CCC(=O)NC3=O)C4=O)CC2)c(Cl)c1)c1ccccn1]

To a solution of benzyl (2-((3-chloro-4-(4-(2-((2-(2,6-dioxopiperidin-3-yl)-1-oxoisoindolin-5-yl) oxy) ethyl) piperazin-1-yl) phenyl) (pyridin-2-yl) amino) ethyl) (methyl)carbamate (110 0 mg, 0.14 mmol) in DCM (1 mL) stirred under nitrogen was added 4N 1,4-dioxane: HCl (0.3 mL) dropwise. The reaction mixture was stirred at 25° C. for 6 hours under N₂. The mixture was filtered with DCM. The filter cake was washed with DCM and dried to dryness to give the product as a solid. The residue was purified by prep-HPLC and lyophilized to give 3-(5-(2-(4-(2-chloro-4-((2-(methylamino) ethyl) (pyridin-2-yl) amino) phenyl) piperazin-1-yl) ethoxy)-1-oxoisoindolin-2-yl) piperidine-2,6-dione (17-1, 50.0 mg, 50.34% yield) as a yellow solid. ESI-MS: m/z=632.2 [M+1]⁺, 316.7 [M/2+1]⁺

### Method 71: Synthesis of Diarylamine Intermediate of Compound 17-1

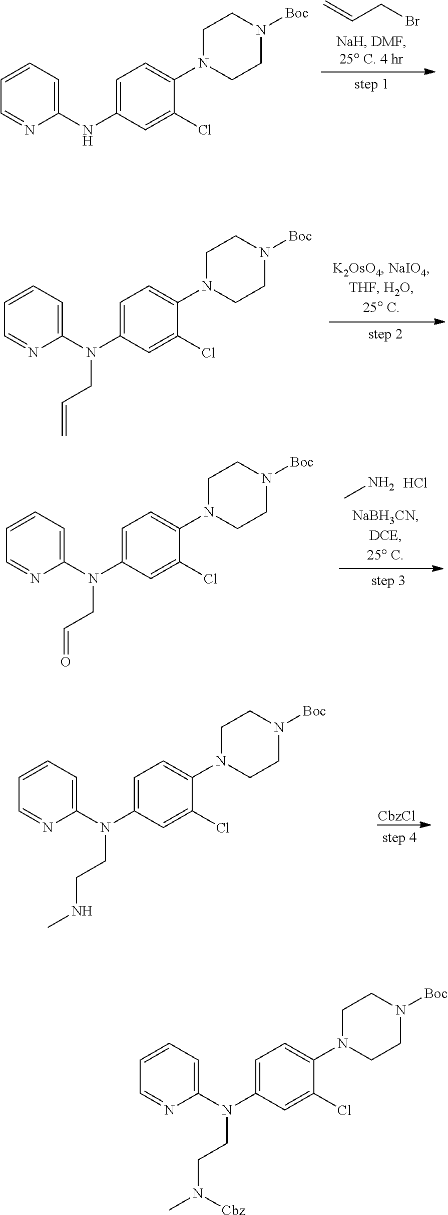

Step 1: To a stirred solution of tert-butyl 4-(2-chloro-4-(pyridin-2-ylamino) phenyl) piperazine-1-carboxylate (2.00 g, 5.1 mmol) in DMF (20 mL) stirred under nitrogen was added NaH (0.41 g, 10.2 mmol) at 0° C. The mixture was stirred at 25° C. for 0.5 hours. Then 3-bromoprop-1-ene (0.93 g, 7.6 mmol) was slowly added dropwise. The reaction was stirred at 25° C. for 6 hours. The reaction mixture was slowly poured into ice water (100 mL) and extracted with EtOAc (100 mL×3). The combined organic layers were washed with brine, dried over Na₂SO₄ and concentrated to give tert-butyl 4-(4-(allyl(pyridin-2-yl) amino)-2-chlorophenyl) piperazine-1-carboxylate (1.00 g, 43.88% yield) as a yellow oil. ESI-MS: m/z=429.1 [M+1]⁺
Step 2: To a stirred solution of tert-butyl 4-(4-(allyl(pyridin-2-yl) amino)-2-chlorophenyl) piperazine-1-carboxylate (1.00 g, 2.3 mmol) in THF/H₂O (3/1, 12 mL) stirred under nitrogen was added Potassium osmate (VI) dihydrate, (10 mg, 0.046 mmol) at 25° C. The mixture was stirred at 25° C. for 0.25 hours. Then Sodium periodate (1.48 g, 6.9 mmol) was added. The reaction was stirred at 25 C for 4 hours. The reaction mixture was poured into water (6 mL) and extracted with DCE (4 mL×3). The combined organic layers were washed with brine, dried over Na₂SO₄ to give tert-butyl 4-(2-chloro-4-((2-oxoethyl) (pyridin-2-yl) amino) phenyl) piperazine-1-carboxylate (crude) as a yellow liquid. ESI-MS: m/z=431.1 [M+1]⁺
Step 3: To a stirred solution of tert-butyl 4-(2-chloro-4-((2-oxoethyl) (pyridin-2-yl) amino) phenyl) piperazine-1-carboxylate (750.0 mg, 1.74 mmol) and Methylamine hydrochloride (175.9 mg, 2.60 mmol) in DCE (12 mL) stirred under nitrogen was added NaBH₃CN (218.2 mg, 3.47 mmol). The reaction was stirred at 25° C. for 2 hours. The reaction mixture was concentrated to give tert-butyl 4-(2-chloro-4-((2-(methylamino) ethyl) (pyridin-2-yl) amino) phenyl) piperazine-1-carboxylate (350.0 mg, 29.69% yield) as a purple solid. ESI-MS: m/z=446.1 [M+1]⁺
Step 4: To a stirred solution of tert-butyl 4-(2-chloro-4-((2-(methylamino) ethyl) (pyridin-2-yl) amino) phenyl) piperazine-1-carboxylate (350.0 mg, 0.78 mmol) and CbzCl (173.6 mg, 1.01 mmol) in DCM (4 mL) stirred under nitrogen was added TEA (237.7 mg, 2.35 mmol). The reaction was stirred 25° C. for 16 hours. The reaction mixture was concentrated to afford a crude product. The crude product was purified by prep-TLC (PE/EtOAc=3/1) to give tert-butyl 4-(4-((2-(((benzyloxy) carbonyl) (methyl) amino) ethyl) (pyridin-2-yl) amino)-2-chlorophenyl) piperazine-1-carboxylate (300.0 mg, 62.92% yield) as a yellow oil. ESI-MS: m/z=580.2 [M+1]⁺

The following compounds 17-1 through 17-5 (42) shown in Table 14 were prepared by methods similar to those described for the preparation of 17-1 (its intermediate), using the appropriate intermediate (41).

TABLE 14

| Compd | 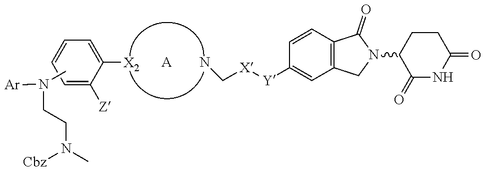 (41) | Product 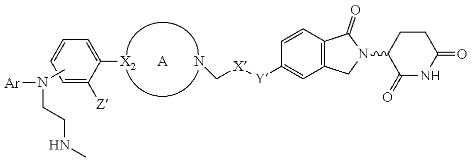 (42) | MS found [M + H]+ |
|---|---|---|---|
| 17-1 | 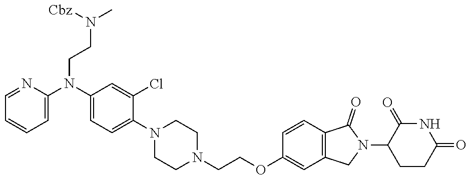 [SMILES: CN(CCN(c1ccc(N2CCN(CCOc3ccc4c(c3)CN(C3CCC(=O)NC3=O)C4=O)CC2)c(Cl)c1)c1ccccn1)C(=O)OCc1ccccc1] | 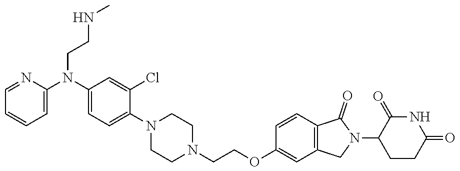 [SMILES: CNCCN(c1ccc(N2CCN(CCOc3ccc4c(c3)CN(C3CCC(=O)NC3=O)C4=O)CC2)c(Cl)c1)c1ccccn1] | 632.2 |
| | | ¹H NMR (400 MHz, MeOD_d4) δ 2.16-2.21 (m, 1H), 2.43- | |
| | | 2.55 (m, 1H), 2.77-2.81 (m, 4H), 2.87-2.97 (m, 1H), | |
| | | 3.32-3.35 (m, 2H), 3.55 (t, J = 10.8 Hz, 2H), 3.67 (d, J = | |
| | | 13.2 Hz, 2H), 3.79-3.88 (m, 4H), 4.27 (t, J = 6.8 Hz, 2H), | |
| | | 4.43-4. 60 (m, 4H), 5.14 (dd, J = 13.2, 5.2 Hz, 1H), 7.03- | |
| | | 7.07 (m, 2H), 7.22 (dd, J = 8.4, 2.2 Hz, 1H), 7.29 (d, J = 1.6 | |
| | | Hz, 1H), 7.42-7.48 (m, 2H), 7.65 (d, J = 1.6 Hz, 1H), 7.78 | |
| | | (d, J = 8.4 Hz, 1H), 7.91-7.96 (m, 1H), 8.07 (d, J = 5.2 Hz, | |
| | | 1H). | |
| 17-2 | 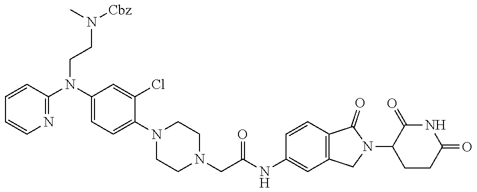 [SMILES: CN(CCN(c1ccc(N2CCN(CC(=O)Nc3ccc4c(c3)CN(C3CCC(=O)NC3=O)C4=O)CC2)c(Cl)c1)c1ccccn1)C(=O)OCc1ccccc1] | 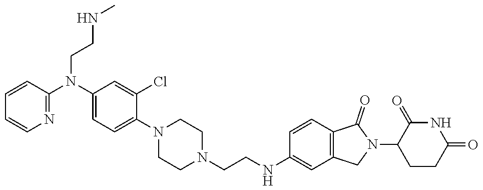 [SMILES: CNCCN(c1ccc(N2CCN(CCNc3ccc4c(c3)CN(C3CCC(=O)NC3=O)C4=O)CC2)c(Cl)c1)c1ccccn1] | 323.3, 645.3 |
| | | ¹H NMR (400 MHz, DMSO_d6) δ 2.12-2.24 (m, 1H), 2.40- | |
| | | 2.56 (m, 1H), 2.78 (s, 3H), 2.79-2.83 (m, 1H), 2.85-2.98 | |
| | | (m, 1H), 3.33-3.43 (m, 4H), 3.52-3.62 (m, 1H), 3.68 (d, J = | |
| | | 12.4 Hz, 2H), 3.87 (d, J = 11.2 Hz, 2H), , 4.30 (t, J = 7.2 | |
| | | Hz, 1H), 4.36 (s,2H), 4.50 (q, J = 17.2 Hz, 1H), 5.15 (dd, J = | |
| | | 13.2, 5.2 Hz, 1H), 7.13 (t, J = 6.6 Hz, 1H), 7.25 (d, J = 9.0 | |
| | | Hz, 1H), 7.45-7.54 (m, 2H), 7.69 (dd, J = 8.6, 1.8 Hz, 2H), | |
| | | 7.79 (d, J = 8.2 Hz, 1H), 7.98-8.14 (m, 3H). | |
| 17-3 | 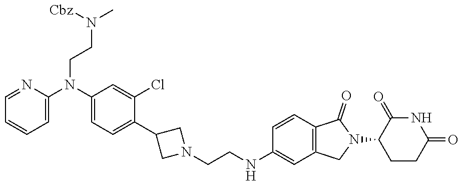 [SMILES: CN(CCN(c1ccc(C2CN(CCNc3ccc4c(c3)CN([C@H]3CCC(=O)NC3=O)C4=O)C2)c(Cl)c1)c1ccccn1)C(=O)OCc1ccccc1] | 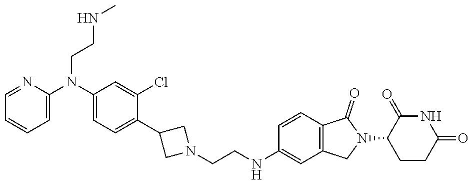 [SMILES: CNCCN(c1ccc(C2CN(CCNc3ccc4c(c3)CN([C@H]3CCC(=O)NC3=O)C4=O)C2)c(Cl)c1)c1ccccn1] | 616.0, 308.6 |
| | | ¹H NMR (400 MHz, DMSO_d6) δ 1.95-2.03 (m, 1H), 2.35- | |
| | | 2.40 (m, 1H), 2.47 (s, 3H), 2.57-2.62 (d, J = 17.6 Hz, 1H), | |
| | | 2.86-2.92 (m, 1H), 2.96 (t, J = 6.4 Hz, 2H), 3.31-3.36 (m, | |
| | | 4H), 3.88-3.93 (m, 2H), 3.94-3.99 (m, 1H), 4.09 (t, J = 6.0 | |
| | | Hz, 2H), 4.29 (d, J = 17.6 Hz, 1H), 4.43 (d, J = 17.6 Hz, 1H), | |
| | | 5.09 (dd, J = 13.2, 5.2 Hz, 1H), 6.45 (d, J = 8.4 Hz, 1H), 6.72- | |
| | | 6.78 (m, 1H), 7.32-7.37 (m, 1H), 7.43-7.50 (m, 2H), | |
| | | 7.59 (d, J = 8.4 Hz, 1H), 7.64-7.72 (m, 2H), 8.02 (s, 1H), | |
| | | 8.18 (d, J = 4.0 Hz, 1H), 8.23 (s, 2H), 10.13 (s, 1H), 11.04 (s, | |
| | | 1H). | |
| 17-4 | 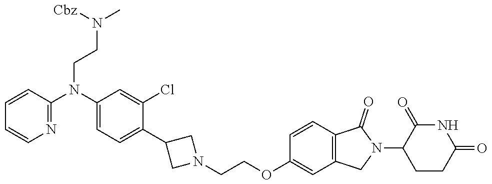 [SMILES: CN(CCN(c1ccc(C2CN(CCOc3ccc4c(c3)CN(C3CCC(=O)NC3=O)C4=O)C2)c(Cl)c1)c1ccccn1)C(=O)OCc1ccccc1] | 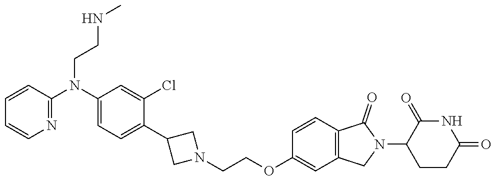 [SMILES: CNCCN(c1ccc(C2CN(CCOc3ccc4c(c3)CN(C3CCC(=O)NC3=O)C4=O)C2)c(Cl)c1)c1ccccn1] | 302.1, 602.9 |
| | | 1H NMR (400 MHz, DMSO_d6) δ 1.94-2.02 (m, 1H), 2.34- | |
| | | 2.42 (m, 1H), 2.55 (s, 3H), 2.59-2.68 (m, 1H), 2.85 (t, J = | |
| | | 5.6 Hz, 2H), 2.88-2.97 (m, 1H), 3.06 (t, J = 6.4 Hz, 2H), | |
| | | 3.23 (t, J = 6.8 Hz, 2H), 3.76-3.88 (m, 3H), 4.04-4.18 (m, | |
| | | 4H), 4.27 (d, J = 17.2 Hz, 1H), 4.39 (d, J = 17.2 Hz, 1H), | |
| | | 5.08 (dd, J = 13.2, 5.2 Hz, 1H), 6.43 (d, J = 8.4 Hz, 1H), 6.77 | |
| | | (dd, J = 6.8, 5.2 Hz, 1H), 7.03-7.08 (m, 1H), 7.17 (d, J = | |
| | | 1.6 Hz, 1H), 7.35 (dd, J = 8.4, 2.0 Hz, 1H), 7.44-7.51 (m, | |
| | | 2H), 7.58 (d, J = 8.3 Hz, 1H), 7.63 (d, J = 8.4 Hz, 1H), 8.17- | |
| | | 8.21 (m, 2H), 10.96 (s, 1H). | |
| 17-5 | 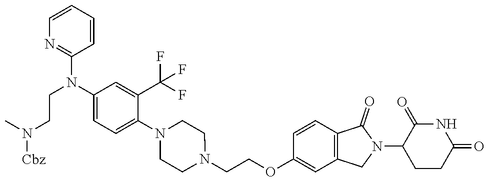 [SMILES: CN(CCN(c1ccc(N2CCN(CCOc3ccc4c(c3)CN(C3CCC(=O)NC3=O)C4=O)CC2)c(C(F)(F)F)c1)c1ccccn1)C(=O)OCc1ccccc1] | 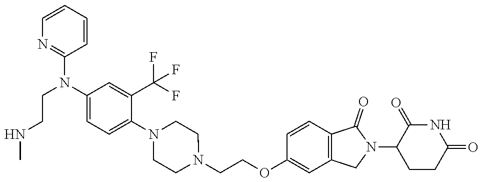 [SMILES: CNCCN(c1ccc(N2CCN(CCOc3ccc4c(c3)CN(C3CCC(=O)NC3=O)C4=O)CC2)c(C(F)(F)F)c1)c1ccccn1] | 333.8, 666.3 |
| | | ¹H NMR (400 MHz, DMSO_d6) δ 1.93-2.03 (m, 1H), 2.35- | |
| | | 2.42 (m, 1H), 2.54-2.69 (m, 4H), 2.85-2.98 (m, 1H), | |
| | | 3.06-3.21 (m, 4H), 3.22-3.35 (m, 2H), 3.43 (t, J = 11.6 | |
| | | Hz, 2H), 3.60-3.78 (m, 4H), 4.17-4.34 (m, 3H), 4.42 (d, J = | |
| | | 17.4 Hz, 1H), 4.60 (s, 2H), 5.09 (dd, J = 13.2, 5.0 Hz, 1H), | |
| | | 6.69 (s, 1H), 6.85-6.94 (m, 1H), 7.16 (dd, J = 8.4, 1.8 Hz, | |
| | | 1H), 7.28 (s, 1H), 7.62-7.72 (m, 3H), 7.81 (d, J = 11.6 Hz, | |
| | | 2H), 8.17 (d, J = 4.4 Hz, 1H), 9.05 (s, 2H), 10.98 (s, 1H), | |
| | | 11.66 (s, 1H). | |

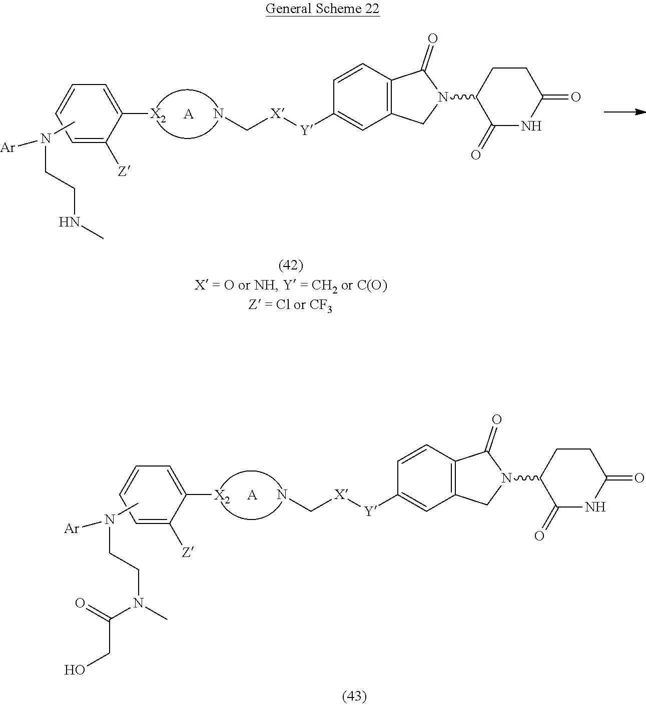

### Method 72

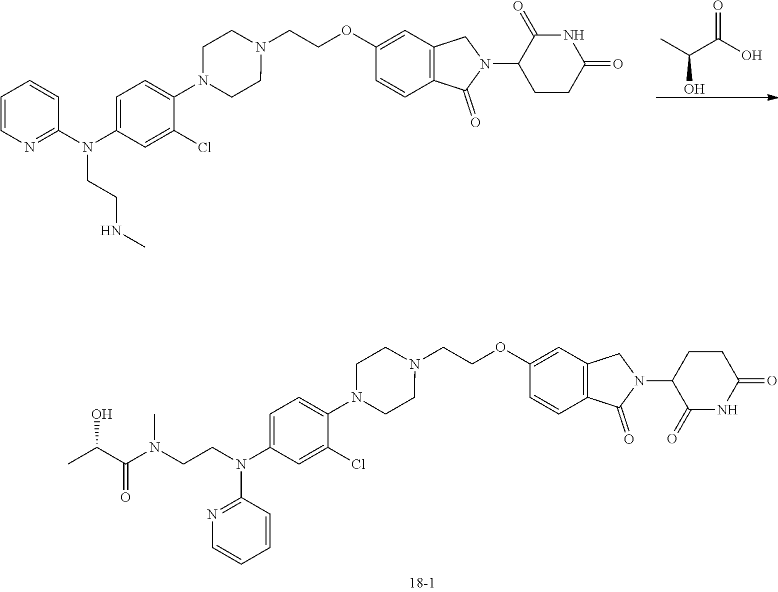

A mixture of 3-[5-[2-[4-[2-chloro-4-[2-(methylamino)ethyl-(2-pyridyl)amino]phenyl]piperazin-1-yl]ethoxy]-1-oxo-isoindolin-2-yl]piperidine-2,6-dione (10 mg, 15.82 μmol), (2S)-2-hydroxypropanoic acid (2.85 mg, 31.64 μmol, 2.36 μL), HATU (9.02 mg, 23.73 μmol), and DIPEA (6.13 mg, 47.46 μmol, 8.27 μL) in DMF (988.05 μL) was shaken for overnight at room temperature. The reaction mixture was diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford compound 18-1 [(2S)—N-[2-[3-chloro-4-[4-[2-[2-(2,6-dioxo-3-piperidyl)-1-oxo-isoindolin-5-yl]oxyethyl]piperazin-1-yl]-N-(2-pyridyl)anilino]ethyl]-2-hydroxy-N-methyl-propanamide (18-1, 5 mg, 7.03 μmol, 44.43% yield, 99% purity)]. EtOAc (10% to 50%) in MeOH. ESI-MS: m/z=704.5 [M+1]⁺

The following compounds 18-1 through 18-4 (43) shown in Table 15 were prepared by methods similar to those described for the preparation of 18-1, using the appropriate intermediate (42).

TABLE 15

| Compd | 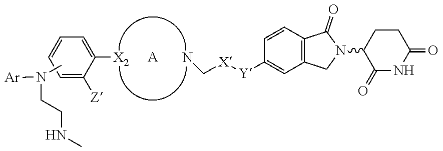 (42) | Product 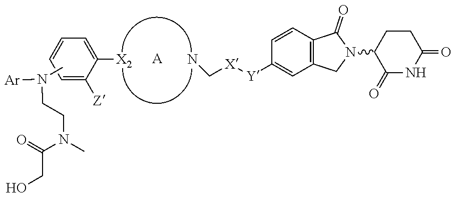 (43) | MS found [M + H]+ |
|---|---|---|---|
| 18-1 | 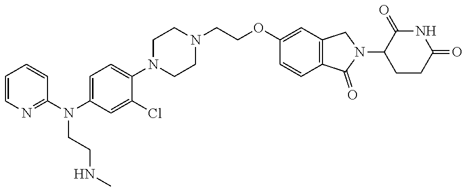 [SMILES: CNCCN(c1ccc(N2CCN(CCOc3ccc4c(c3)CN(C3CCC(=O)NC3=O)C4=O)CC2)c(Cl)c1)c1ccccn1] | 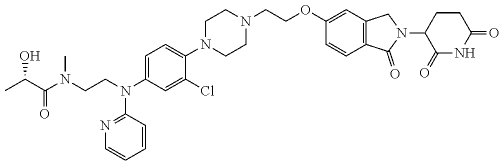 [SMILES: C[C@H](O)C(=O)N(C)CCN(c1ccc(N2CCN(CCOc3ccc4c(c3)CN(C3CCC(=O)NC3=O)C4=O)CC2)c(Cl)c1)c1ccccn1] | 352.8, 704.5 |
| 18-2 | 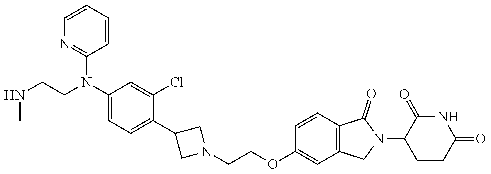 [SMILES: CNCCN(c1ccc(C2CN(CCOc3ccc4c(c3)CN(C3CCC(=O)NC3=O)C4=O)C2)c(Cl)c1)c1ccccn1] | 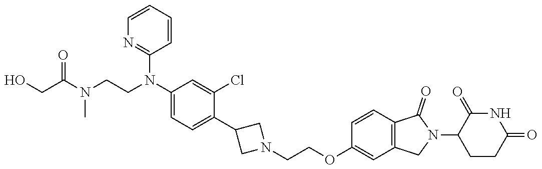 [SMILES: CN(CCN(c1ccc(C2CN(CCOc3ccc4c(c3)CN(C3CCC(=O)NC3=O)C4=O)C2)c(Cl)c1)c1ccccn1)C(=O)CO] | 661.5, 331.1 |
| 18-3 | 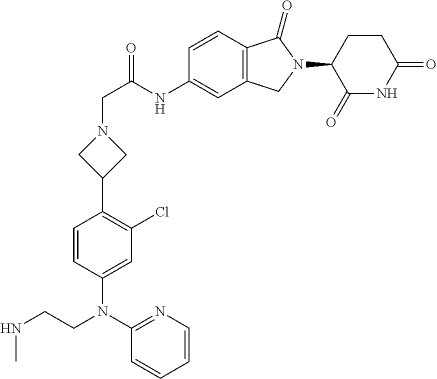 [SMILES: CNCCN(c1ccc(C2CN(CC(=O)Nc3ccc4c(c3)CN([C@H]3CCC(=O)NC3=O)C4=O)C2)c(Cl)c1)c1ccccn1] | 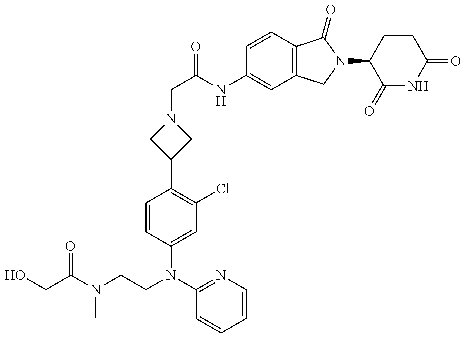 [SMILES: CN(CCN(c1ccc(C2CN(CC(=O)Nc3ccc4c(c3)CN([C@H]3CCC(=O)NC3=O)C4=O)C2)c(Cl)c1)c1ccccn1)C(=O)CO] | 674.5, 337.7 |
| 18-4 | 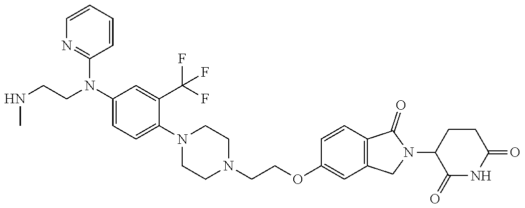 [SMILES: CNCCN(c1ccc(N2CCN(CCOc3ccc4c(c3)CN(C3CCC(=O)NC3=O)C4=O)CC2)c(C(F)(F)F)c1)c1ccccn1] | 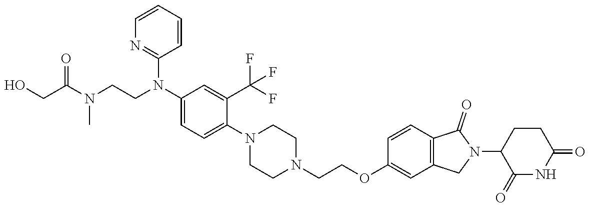 [SMILES: CN(CCN(c1ccc(N2CCN(CCOc3ccc4c(c3)CN(C3CCC(=O)NC3=O)C4=O)CC2)c(C(F)(F)F)c1)c1ccccn1)C(=O)CO] | 724.5, 362.8 |

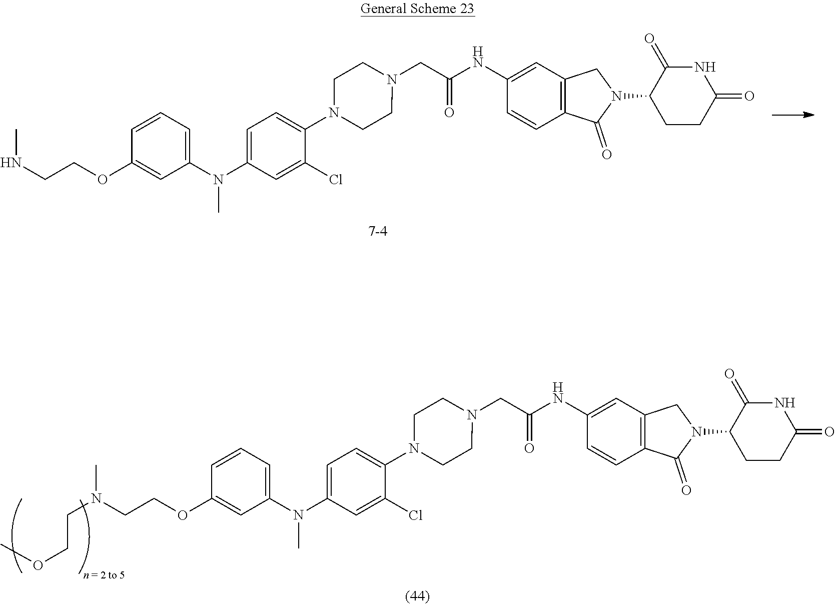

### Method 73

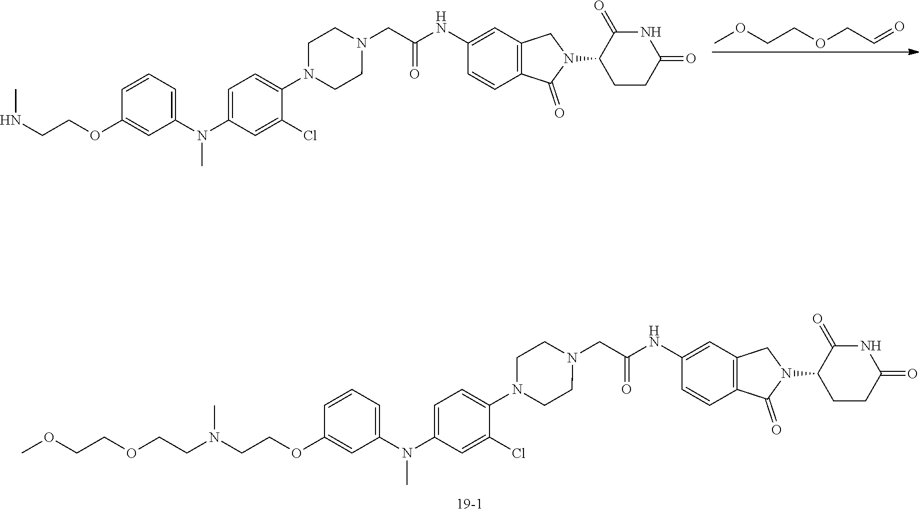

A mixture of 2-[4-[2-chloro-4-[N-methyl-3-[2-(methylamino)ethoxy]anilino]phenyl]piperazin-1-yl]-N-[2-[(3S)-2,6-dioxo-3-piperidyl]-1-oxo-isoindolin-5-yl]acetamide (20 mg, 29.67 μmol), 2-(2-methoxyethoxy)acetaldehyde (7.01 mg, 59.33 μmol, 7.19 μL), and Acetic acid (1.78 mg, 29.67 μmol) in THF (492.82 μL) was treated with sodium;triacetoxyboranuide (12.57 mg, 59.33 μmol) at room temperature, then the mixture was shaken for 4 hours. The reaction mixture was diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford compound 19-1 [2-[4-[2-chloro-4-[3-[2-[2-(2-methoxyethoxy)ethyl-methyl-amino]ethoxy]-N-methyl-anilino]phenyl]piperazin-1-yl]-N-[2-[(3S)-2,6-dioxo-3-piperidyl]-1-oxo-isoindolin-5-yl]acetamide (19-1, 8.5 mg, 10.73 μmol, 36.17% yield, 98% purity)]. EtOAc (0% to 40%) in MeOH. ESI-MS: m/z=776.5 [M+1]⁺.

The following compounds 19-1 through 19-4 (44) shown in Table 16 were prepared by methods similar to those described for the preparation of 19-1, using the appropriate intermediate (7-4).

TABLE 16

| Compd | 7-4 | Product 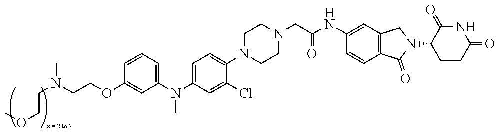 (44) | MS found [M + H]+ |
|---|---|---|---|
| 19-1 | 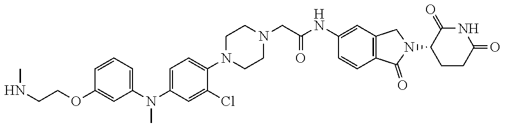 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 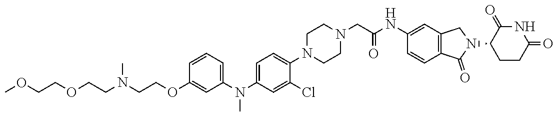 [SMILES: COCCOCCN(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 776.5, 388.8 |
| 19-2 | 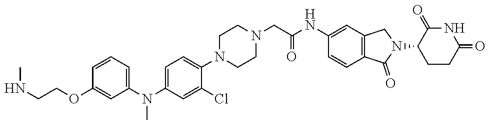 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 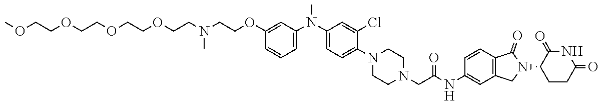 [SMILES: COCCOCCOCCOCCN(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 432.9 |
| 19-3 | 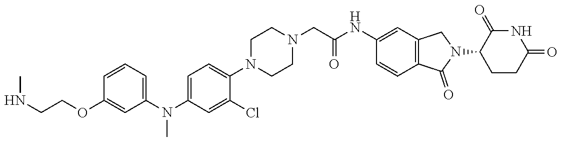 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 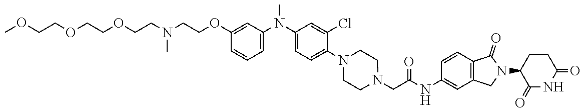 [SMILES: COCCOCCOCCN(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 410.6 |
| 19-4 | 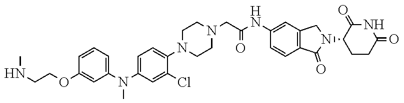 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 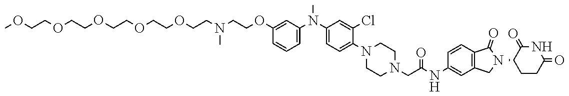 [SMILES: COCCOCCOCCOCCOCCN(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 454.9 |

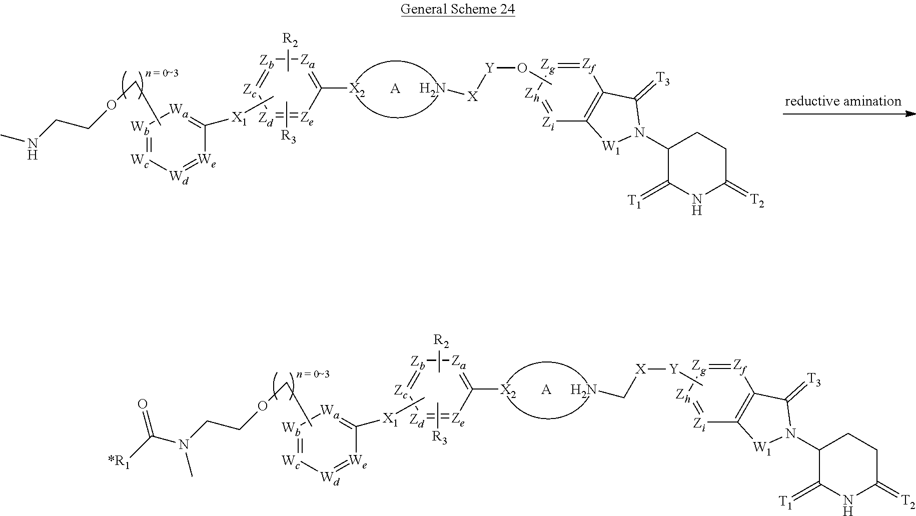

### Methods 74

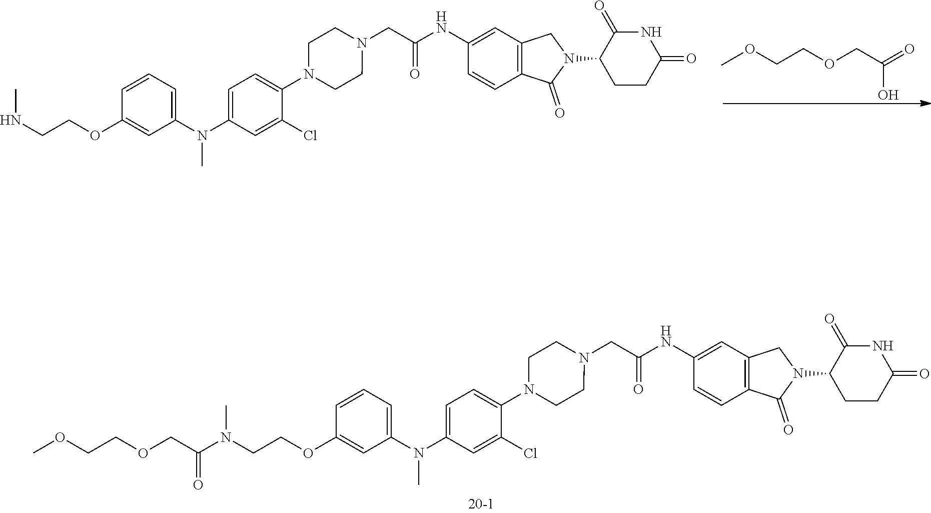

A mixture of 2-[4-[2-chloro-4-[N-methyl-3-[2-(methylamino)ethoxy]anilino]phenyl]piperazin-1-yl]-N-[2-[(3S)-2,6-dioxo-3-piperidyl]-1-oxo-isoindolin-5-yl]acetamide (20 mg, 29.67 μmol), 2-(2-methoxyethoxy)acetic acid (7.96 mg, 59.33 μmol), HATU (16.92 mg, 44.50 μmol), and DIPEA (11.50 mg, 89.00 μmol, 15.50 μL) in DMF (981.57 μL) was shaken for 4 hours at room temperature. The reaction mixture was diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford compound 19-1 [2-[4-[2-chloro-4-[3-[2-[[2-(2-methoxyethoxy)acetyl]-methyl-amino]ethoxy]-N-methyl-anilino]phenyl]piperazin-1-yl]-N-[2-[(3 S)-2,6-dioxo-3-piperidyl]-1-oxo-isoindolin-5-yl]acetamide (20-1, 17 mg, 20.01 μmol, 67.44% yield, 93% purity)]. EtOAc (0% to 40%) in MeOH. ESI-MS: m/z=681.8 [M+1]⁺/2.

The following compounds 20-1 through 20-6 (45) shown in Table 17 were prepared by methods similar to those described for the preparation of 20-1, using the appropriate intermediate (7-4 or 7-44).

TABLE 17

| Compd | 7-4 or 7-44 | Product 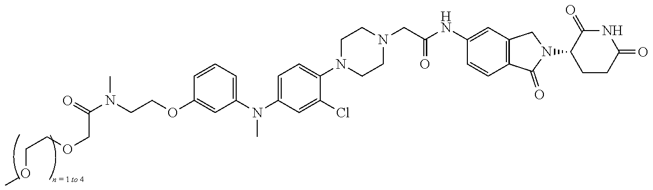 (45) | MS found [M + H]+ |
|---|---|---|---|
| 20-1 | 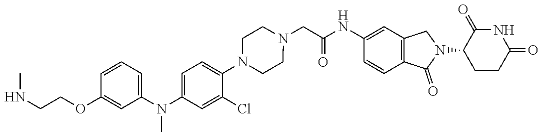 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 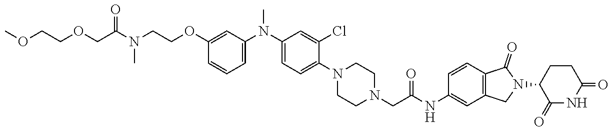 [SMILES: COCCOCC(=O)N(C)CCOc1cccc(N(C)c2ccc(N3CCN(CC(=O)Nc4ccc5c(c4)CN([C@@H]4CCC(=O)NC4=O)C5=O)CC3)c(Cl)c2)c1] | 491.4 |
| 20-2 | 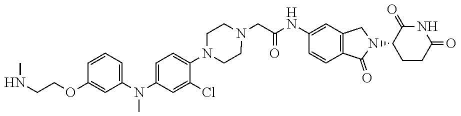 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 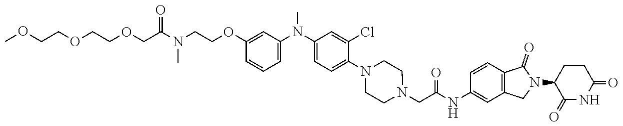 [SMILES: COCCOCCOCC(=O)N(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 417.9 |
| 20-3 | 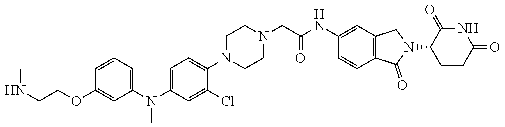 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 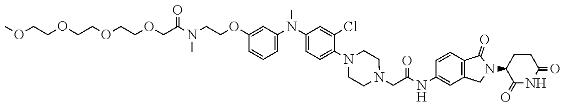 [SMILES: COCCOCCOCCOCC(=O)N(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 439.9 |
| 20-4 | 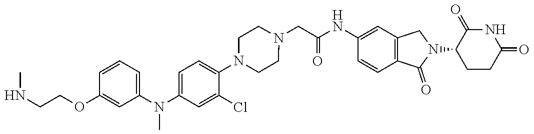 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 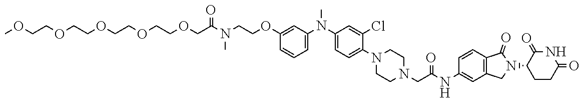 [SMILES: COCCOCCOCCOCCOCC(=O)N(C)CCOc1cccc(N(C)c2ccc(N3CCN(CC(=O)Nc4ccc5c(c4)CN([C@H]4CCC(=O)NC4=O)C5=O)CC3)c(Cl)c2)c1] | 462.1 |
| 20-5 | 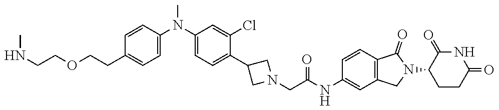 [SMILES: CNCCOCCc1ccc(N(C)c2ccc(C3CN(CC(=O)Nc4ccc5c(c4)CN([C@H]4CCC(=O)NC4=O)C5=O)C3)c(Cl)c2)cc1] | 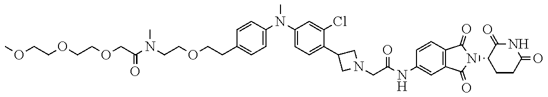 [SMILES: COCCOCCOCC(=O)N(C)CCOCCc1ccc(N(C)c2ccc(C3CN(CC(=O)Nc4ccc5c(c4)C(=O)N([C@H]4CCC(=O)NC4=O)C5=O)C3)c(Cl)c2)cc1] | 833.5, 417.2 |
| 20-6 | 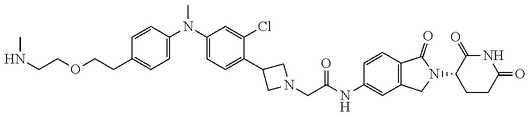 [SMILES: CNCCOCCc1ccc(N(C)c2ccc(C3CN(CC(=O)Nc4ccc5c(c4)CN([C@H]4CCC(=O)NC4=O)C5=O)C3)c(Cl)c2)cc1] | 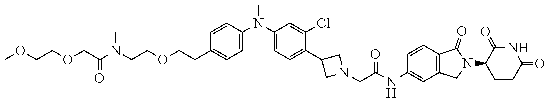 [SMILES: COCCOCC(=O)N(C)CCOCCc1ccc(N(C)c2ccc(C3CN(CC(=O)Nc4ccc5c(c4)CN([C@@H]4CCC(=O)NC4=O)C5=O)C3)c(Cl)c2)cc1] | 789.5, 396.3 |

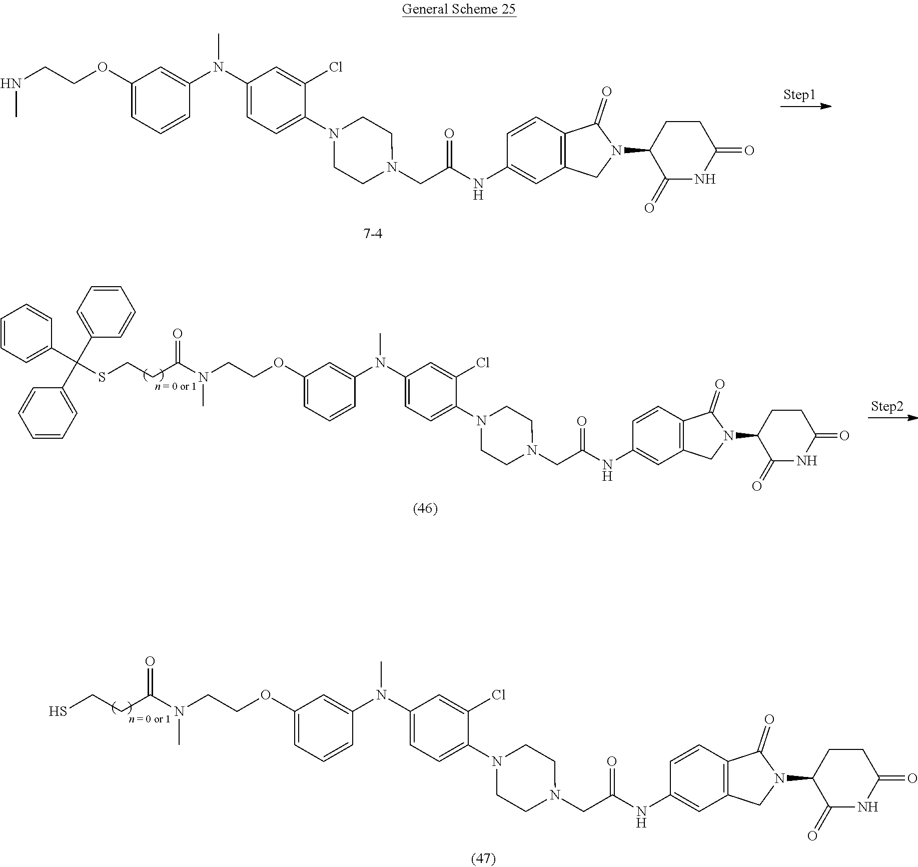

### Method 75

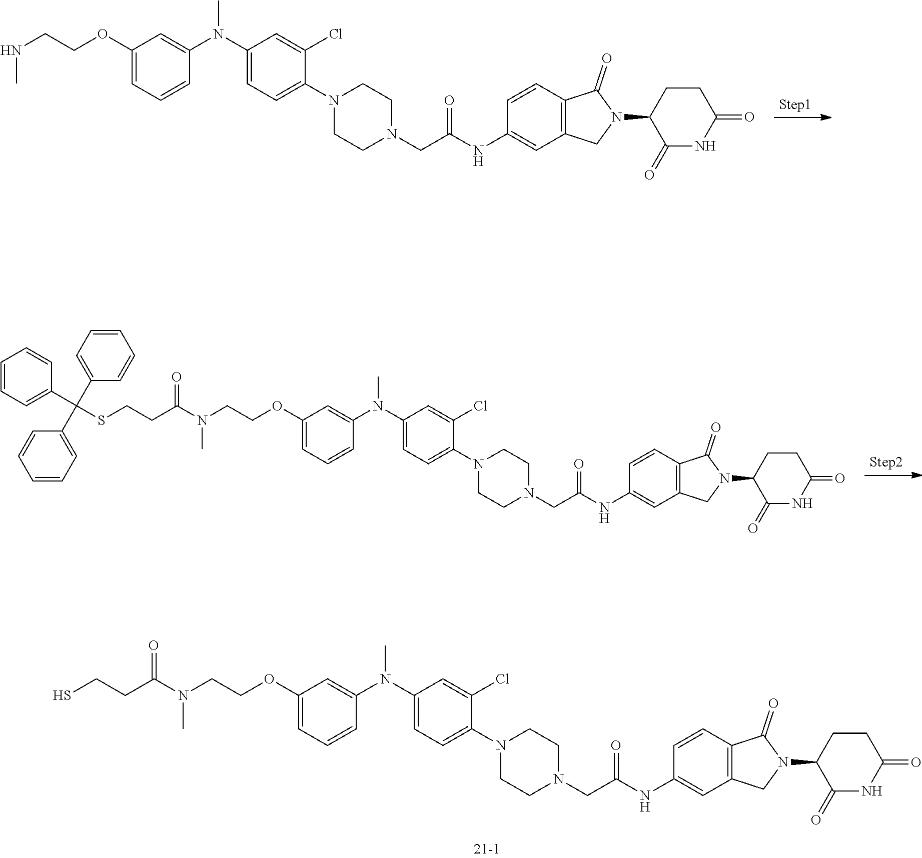 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1 ; CN(CCOc1cccc(N(C)c2ccc(N3CCN(CC(=O)Nc4ccc5c(c4)CN([C@H]4CCC(=O)NC4=O)C5=O)CC3)c(Cl)c2)c1)C(=O)CCSC(c1ccccc1)(c1ccccc1)c1ccccc1 ; CN(CCOc1cccc(N(C)c2ccc(N3CCN(CC(=O)Nc4ccc5c(c4)CN([C@H]4CCC(=O)NC4=O)C5=O)CC3)c(Cl)c2)c1)C(=O)CCS]

Step 1: A mixture of 2-[4-[2-chloro-4-[N-methyl-3-[2-(methylamino) ethoxy]anilino]phenyl]piperazin-1-yl]-N-[2-[(3S)-2,6-dioxo-3-piperidyl]-1-oxo-isoindolin-5-yl]acetamide (30.00 mg, 44.50 μmol) and (2,5-dioxopyrrolidin-1-yl) 3-tritylsulfanylpropanoate (43.62 mg, 97.90 μmol) in DMF (958.04 μL) was treated with DIPEA (23.00 mg, 177.99 μmol, 31.00 μL) at room temperature, and then the reaction mixture was shaken for 2 hours. The reaction mixture was diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford compound [N-[2-[3-[3-chloro-4-[4-[2-[[2-[(3S)-2,6-dioxo-3-piperidyl]-1-oxo-isoindolin-5-yl]amino]-2-oxo-ethyl]piperazin-1-yl]-N-methyl-anilino]phenoxy]ethyl]-N-methyl-3-tritylsulfanyl-propanamide (33 mg, 32.85 μmol, 74% yield)]. EtOAc (0% to 40%) in MeOH. ESI-MS: m/z=503.3 [M+1]⁺/2
Step 2: A solution of N-[2-[3-[3-chloro-4-[4-[2-[[2-[(3S)-2,6-dioxo-3-piperidyl]-1-oxo-isoindolin-5-yl]amino]-2-oxo-ethyl]piperazin-1-yl]-N-methyl-anilino]phenoxy]ethyl]-N-methyl-3-tritylsulfanyl-propanamide (35 mg, 34.84 μmol) in TFA was treated with triethyl silane (4.05 mg, 34.84 μmol, 5.56 μL) at room temperature, and then the reaction mixture was shaken for overnight. The reaction mixture was diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford compound 21-1 [N-[2-[3-[3-chloro-4-[4-[2-[[2-[(3S)-2,6-dioxo-3-piperidyl]-1-oxo-isoindolin-5-yl]amino]-2-oxo-ethyl]piperazin-1-yl]-N-methyl-anilino]phenoxy]ethyl]-N-methyl-3-sulfanyl-propanamide (21-1, 24 mg, 31.48 μmol, 90.37% yield, 100% purity). EtOAc (0% to 40%) in MeOH. ESI-MS: m/z=751.6 [M+1]⁺.

The following compounds 21-1 through 21-2 (47) shown in Table 18 were prepared by methods similar to those described for the preparation of 21-1, using the appropriate intermediate (7-4).

TABLE 18

| Compd | 7-4 | Product 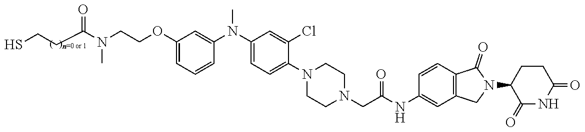 (47) | MS found [M + H]+ |
|---|---|---|---|
| 21-1 | 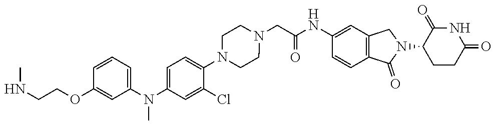 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 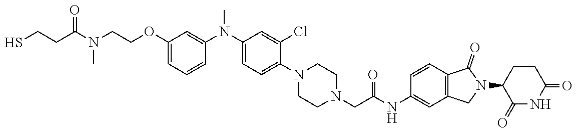 [SMILES: CN(CCOc1cccc(N(C)c2ccc(N3CCN(CC(=O)Nc4ccc5c(c4)CN([C@H]4CCC(=O)NC4=O)C5=O)CC3)c(Cl)c2)c1)C(=O)CCS] | 491.4 |
| 21-2 | 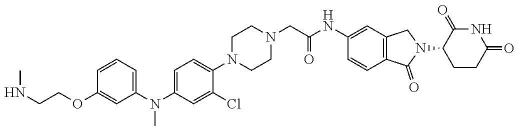 [SMILES: CNCCOc1cccc(N(C)c2ccc(N3CCN(CC(=O)Nc4ccc5c(c4)CN([C@H]4CCC(=O)NC4=O)C5=O)CC3)c(Cl)c2)c1] | 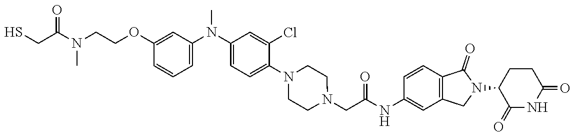 [SMILES: CN(CCOc1cccc(N(C)c2ccc(N3CCN(CC(=O)Nc4ccc5c(c4)CN([C@@H]4CCC(=O)NC4=O)C5=O)CC3)c(Cl)c2)c1)C(=O)CS] | 417.9 |

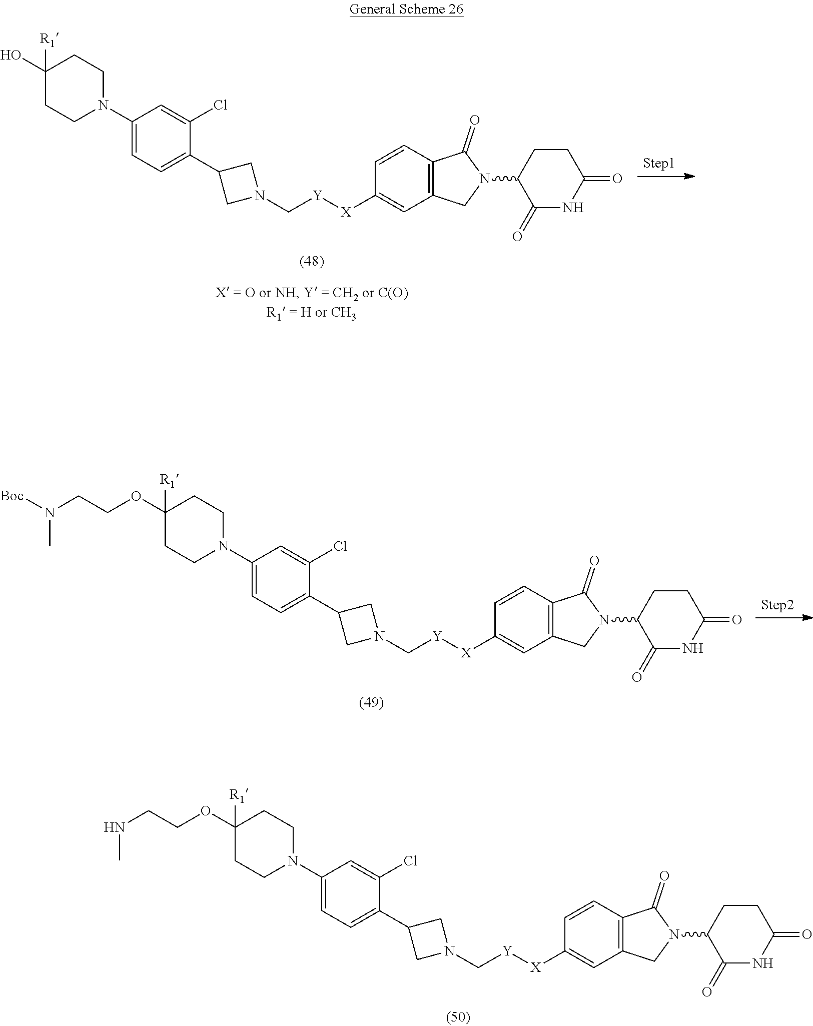

### Method 76

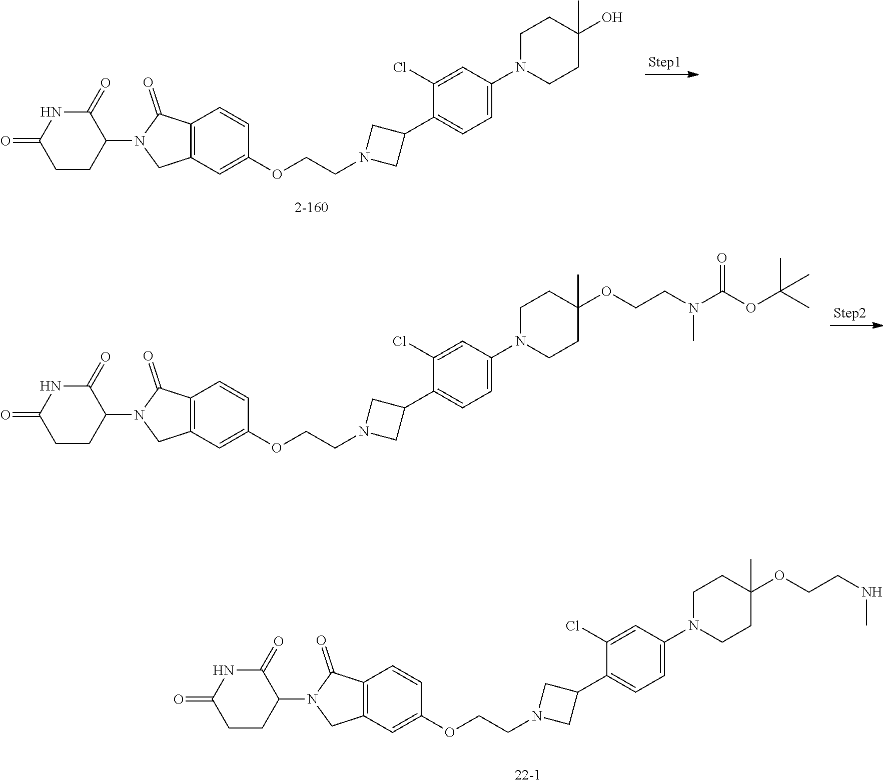 [SMILES: CC1(O)CCN(c2ccc(C3CN(CCOc4ccc5c(c4)CN(C4CCC(=O)NC4=O)C5=O)C3)c(Cl)c2)CC1 ; CN(CCOC1(C)CCN(c2ccc(C3CN(CCOc4ccc5c(c4)CN(C4CCC(=O)NC4=O)C5=O)C3)c(Cl)c2)CC1)C(=O)OC(C)(C)C ; CNCCOC1(C)CCN(c2ccc(C3CN(CCOc4ccc5c(c4)CN(C4CCC(=O)NC4=O)C5=O)C3)c(Cl)c2)CC1]

Step 1: The mixture of 3-[5-[2-[3-[2-chloro-4-(4-hydroxy-4-methyl-1-piperidyl)phenyl]azetidin-1-yl]ethoxy]-1-oxo-isoindolin-2-yl]piperidine-2,6-dione (40 mg, 70.54 μmol) tert-butyl N-(2-chloroethyl)-N-methyl-carbamate (2-160, 27.32 mg, 141.07 μmol, 24.84 μL), and K₂CO₃ (29.25 mg, 211.61 μmol) in DMF (978.27 μL) was heated to 80° C. and shaken for 1 hour. The reaction mixture was cooled to room temperature and diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford compound {tert-butyl N-[2-[[1-[3-chloro-4-[1-[2-[2-(2,6-dioxo-3-piperidyl)-1-oxo-isoindolin-5-yl]oxyethyl]azetidin-3-yl]phenyl]-4-methyl-4-piperidyl]oxy]ethyl]-N-methyl-carbamate (35 mg, 48.32 μmol, 68.51% yield). EtOAc (0% to 40%)} in MeOH. ESI-MS: m/z=725.1 [M+1]⁺
Step 2: A solution of tert-butyl N-[2-[[1-[3-chloro-4-[1-[2-[2-(2,6-dioxo-3-piperidyl)-1-oxo-isoindolin-5-yl]oxyethyl]azetidin-3-yl]phenyl]-4-methyl-4-piperidyl]oxy]ethyl]-N-methyl-carbamate (35 mg, 48.32 μmol) in DCM (3 mL) was treated with TFA (0.5 mL) at room temperature. The reaction mixture was shaken 1 h, and then concentrated, and purified by a flash column to afford compound 22-1 {tert-butyl N-[2-[[1-[3-chloro-4-[1-[2-[2-(2,6-dioxo-3-piperidyl)-1-oxo-isoindolin-5-yl]oxyethyl]azetidin-3-yl]phenyl]-4-methyl-4-piperidyl]oxy]ethyl]-N-methyl-carbamate (35 mg, 48.32 mol, 68.51% yield)}. EtOAc (0% to 40%) in MeOH. ESI-MS: m/z=312.7 [M+1]⁺/2.

The following compounds 22-1 through 22-3 (50) shown in Table 19 were prepared by methods similar to those described for the preparation of 22-1, using the appropriate intermediate (48).

TABLE 19

| Compd | 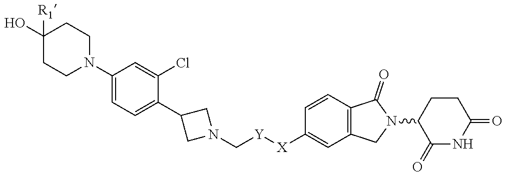 (48) | Product 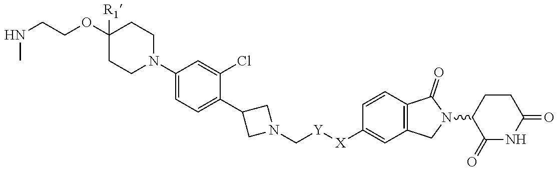 (50) | MS found [M + H]+ |
|---|---|---|---|
| 22-1 | 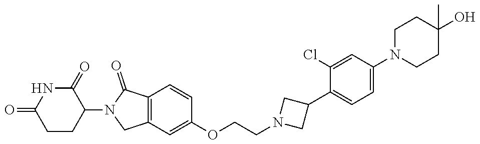 [SMILES: CC1(O)CCN(c2ccc(C3CN(CCOc4ccc5c(c4)CN(C4CCC(=O)NC4=O)C5=O)C3)c(Cl)c2)CC1] | 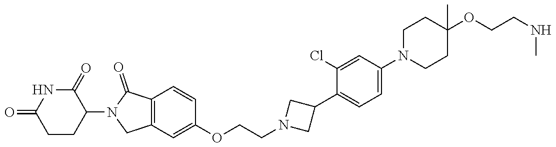 [SMILES: CNCCOC1(C)CCN(c2ccc(C3CN(CCOc4ccc5c(c4)CN(C4CCC(=O)NC4=O)C5=O)C3)c(Cl)c2)CC1] | 312.7 |
| 22-2 | 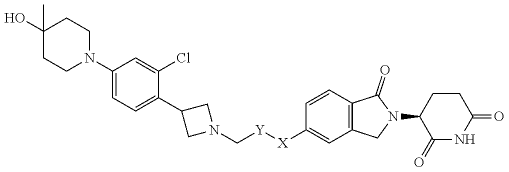 | 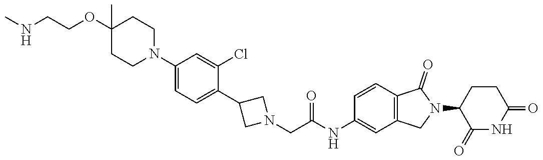 [SMILES: CNCCOC1(C)CCN(c2ccc(C3CN(CC(=O)Nc4ccc5c(c4)CN([C@H]4CCC(=O)NC4=O)C5=O)C3)c(Cl)c2)CC1] | 319.1, 637.5 |
| 22-3 | 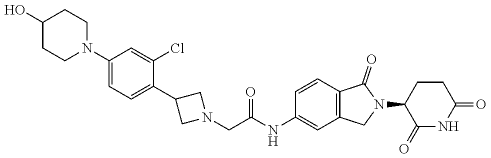 [SMILES: O=C1CC[C@H](N2Cc3cc(NC(=O)CN4CC(c5ccc(N6CCC(O)CC6)cc5Cl)C4)ccc3C2=O)C(=O)N1] | 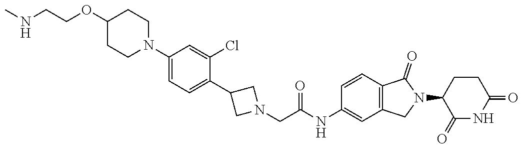 [SMILES: CNCCOC1CCN(c2ccc(C3CN(CC(=O)Nc4ccc5c(c4)CN([C@H]4CCC(=O)NC4=O)C5=O)C3)c(Cl)c2)CC1] | 312.2 |

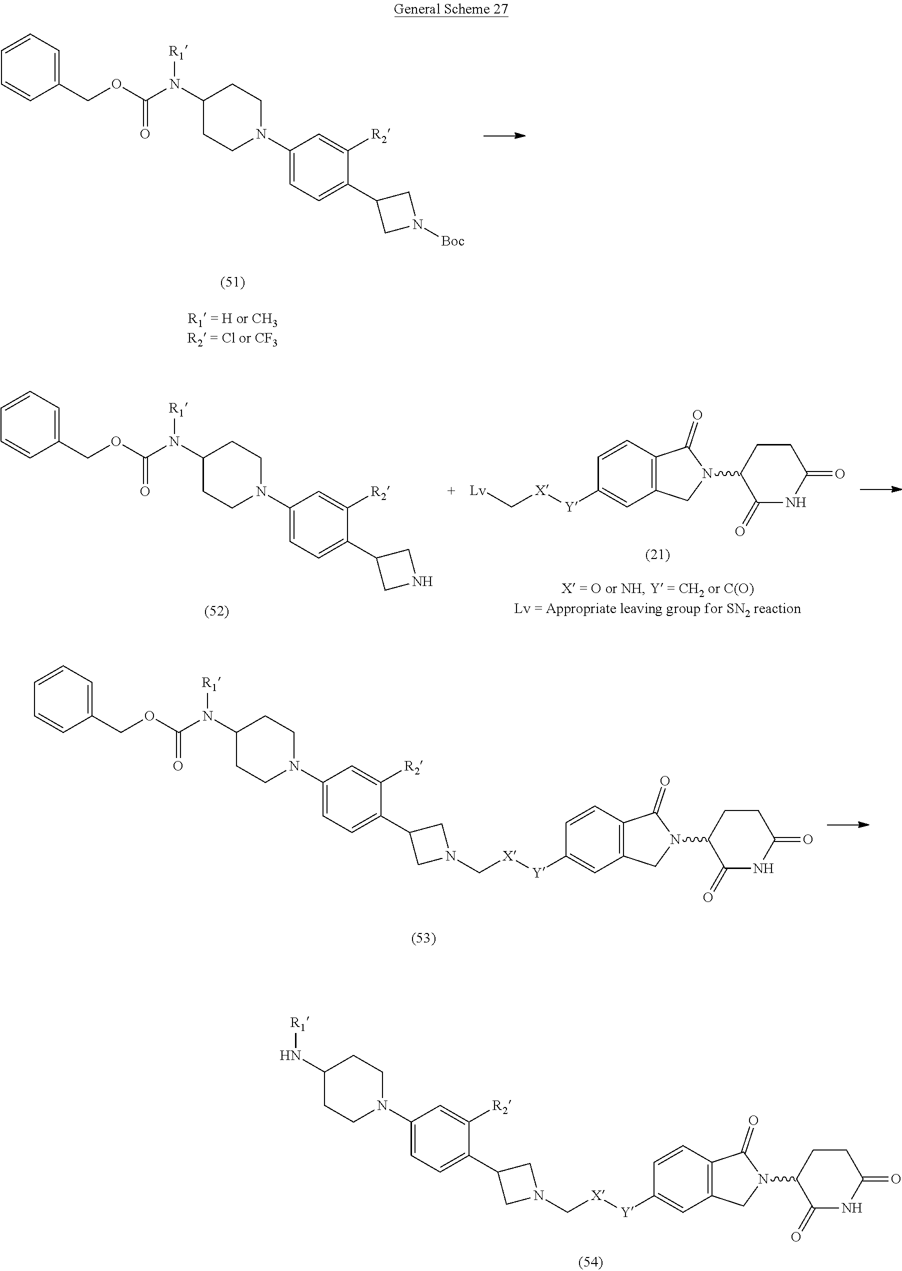

### Method 77

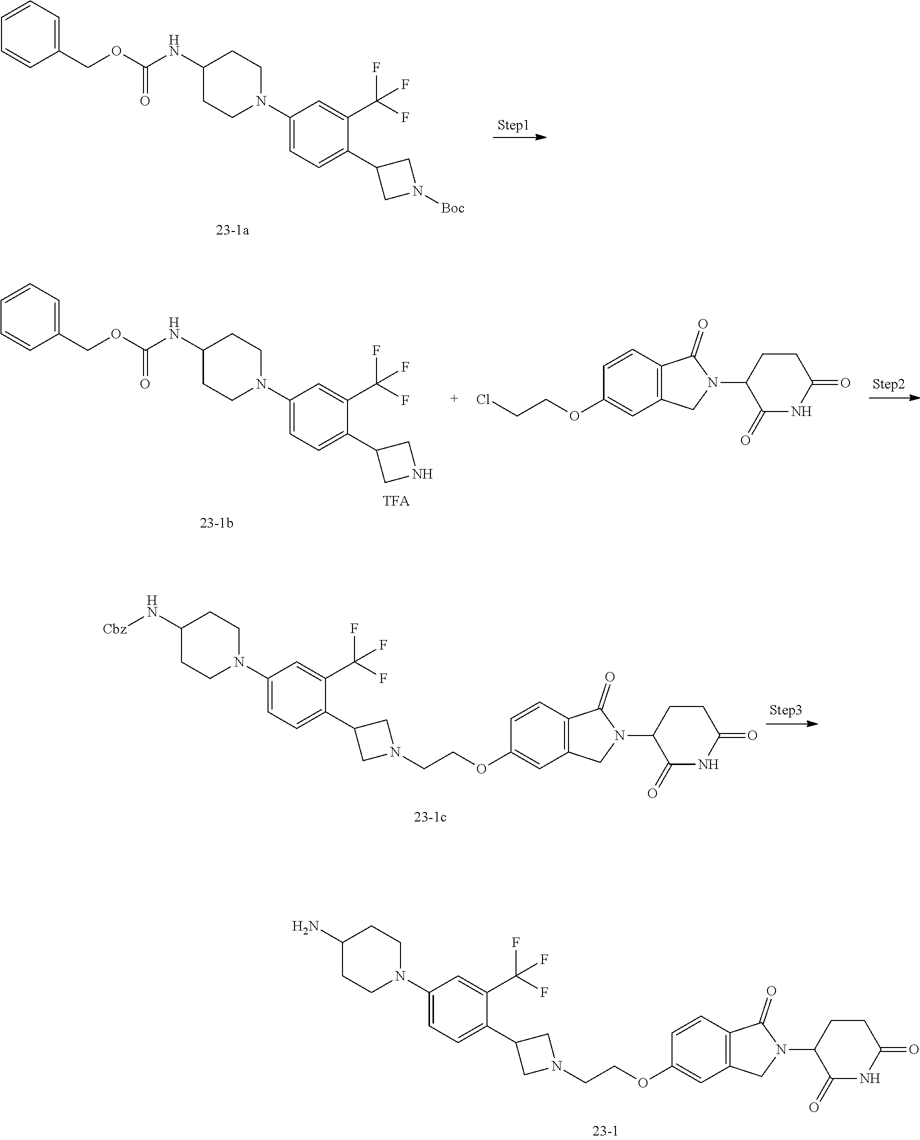 [SMILES: CC(C)(C)OC(=O)N1CC(c2ccc(N3CCC(NC(=O)OCc4ccccc4)CC3)cc2C(F)(F)F)C1 ; O=C(NC1CCN(c2ccc(C3CNC3)c(C(F)(F)F)c2)CC1)OCc1ccccc1 ; O=C1CCC(N2Cc3cc(OCCCl)ccc3C2=O)C(=O)N1 ; O=C1CCC(N2Cc3cc(OCCN4CC(c5ccc(N6CCC(NC(=O)OCc7ccccc7)CC6)cc5C(F)(F)F)C4)ccc3C2=O)C(=O)N1 ; NC1CCN(c2ccc(C3CN(CCOc4ccc5c(c4)CN(C4CCC(=O)NC4=O)C5=O)C3)c(C(F)(F)F)c2)CC1]

Step 1: A solution of tert-butyl 3-[4-[4-(benzyloxycarbonylamino)-1-piperidyl]-2-(trifluoromethyl)phenyl]azetidine-1-carboxylate (23-1a, 150 mg, 281.12 μmol) in DCM (2 mL) was treated with TFA (0.5 mL) at a room temperature, then the reaction mixture was shaken for 1 hour and concentrated to afford benzyl N-[1-[4-(azetidin-3-yl)-3-(trifluoromethyl)phenyl]-4-piperidyl]carbamate (23-1b, crude). No further purification. ESI-MS: m/z=434.3 [M+1]⁺.
Step 2: A mixture of 3-[5-(2-chloroethoxy)-1-oxo-isoindolin-2-yl]piperidine-2,6-dione (232.70 mg, 720.99 μmol), benzyl N-[1-[4-(azetidin-3-yl)-3-(trifluoromethyl)phenyl]-4-piperidyl]carbamate (260.44 mg, 600.83 μmol), K₂CO₃ (332.15 mg, 2.40 mmol), and NaI (90.060 mg, 600.83 μmol, 24.54 μL) in DMF (1.97 mL) was heated to 80° C. and shaken for 10 hours. The reaction mixture was cooled to a room temperature, then diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford benzyl N-[1-[4-[1-[2-[2-(2,6-dioxo-3-piperidyl)-1-oxo-isoindolin-5-yl]oxyethyl]azetidin-3-yl]-3-(trifluoromethyl)phenyl]-4-piperidyl]carbamate (23-1c). MeOH (5 to 50%) in EtOAc. ESI-MS: m/z=810.3 [M+1]⁺.
Step 3: A solution of benzyl N-[1-[4-[1-[2-[2-(2,6-dioxo-3-piperidyl)-1-oxo-isoindolin-5-yl]oxyethyl]azetidin-3-yl]-3-(trifluoromethyl)phenyl]-4-piperidyl]carbamate (194.18 mg, 269.78 μmol) in TFA (1 mL) was heated to 50° C. and shaken for 2 hours. The solution was cooled to a room temperature, concentrated, diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford 3-[5-[2-[3-[4-(4-amino-1-piperidyl)-2-(trifluoromethyl)phenyl]azetidin-1-yl]ethoxy]-1-oxo-isoindolin-2-yl]piperidine-2,6-dione (23-1, 73 mg, 64.82 μmol, 24% yield, 52% purity). MeOH (10 to 60%) in EtOAc. ESI-MS: m/z=298.8 [M+1]⁺/2.

The following compounds 23-1 through 23-6 (54) shown in Table 20 were prepared by methods similar to those described for the preparation of 23-1, using the appropriate intermediates (51) and (21).

TABLE 20

| Compd | | |
|---|---|---|
| | 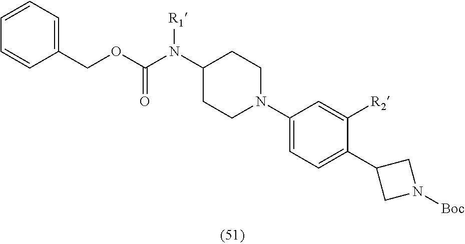 | 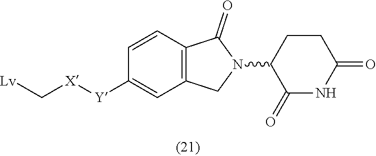 |
| 23-1 | 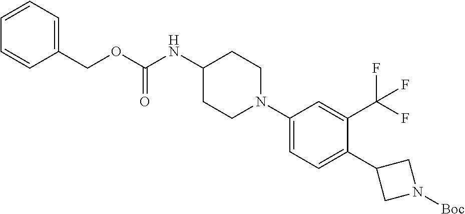 [SMILES: CC(C)(C)OC(=O)N1CC(c2ccc(N3CCC(NC(=O)OCc4ccccc4)CC3)cc2C(F)(F)F)C1] | 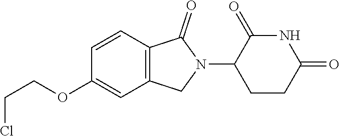 [SMILES: O=C1CCC(N2Cc3cc(OCCCl)ccc3C2=O)C(=O)N1] |
| 23-2 | 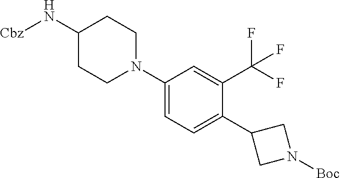 [SMILES: CC(C)(C)OC(=O)N1CC(c2ccc(N3CCC(NC(=O)OCc4ccccc4)CC3)cc2C(F)(F)F)C1] | 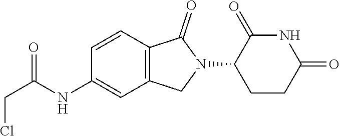 [SMILES: O=C1CC[C@H](N2Cc3cc(NC(=O)CCl)ccc3C2=O)C(=O)N1] |
| 23-3 | 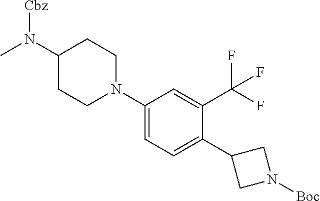 [SMILES: CN(C(=O)OCc1ccccc1)C1CCN(c2ccc(C3CN(C(=O)OC(C)(C)C)C3)c(C(F)(F)F)c2)CC1] | 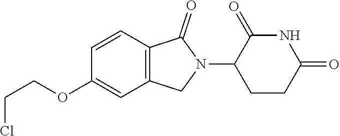 [SMILES: O=C1CCC(N2Cc3cc(OCCCl)ccc3C2=O)C(=O)N1] |
| 23-4 | 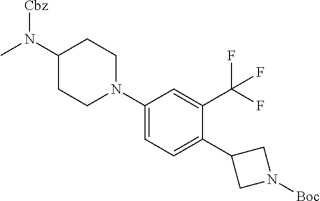 [SMILES: CN(C(=O)OCc1ccccc1)C1CCN(c2ccc(C3CN(C(=O)OC(C)(C)C)C3)c(C(F)(F)F)c2)CC1] | 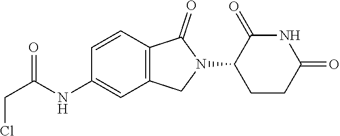 [SMILES: O=C1CC[C@H](N2Cc3cc(NC(=O)CCl)ccc3C2=O)C(=O)N1] |
| 23-5 | 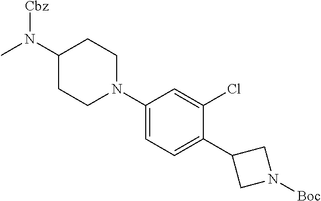 [SMILES: CN(C(=O)OCc1ccccc1)C1CCN(c2ccc(C3CN(C(=O)OC(C)(C)C)C3)c(Cl)c2)CC1] | 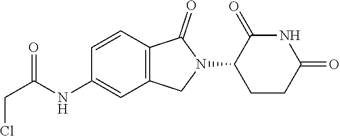 [SMILES: O=C1CC[C@H](N2Cc3cc(NC(=O)CCl)ccc3C2=O)C(=O)N1] |
| 23-6 | 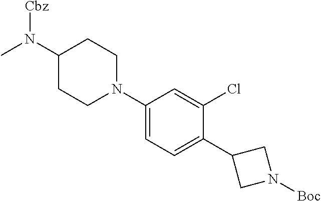 [SMILES: CN(C(=O)OCc1ccccc1)C1CCN(c2ccc(C3CN(C(=O)OC(C)(C)C)C3)c(Cl)c2)CC1] | 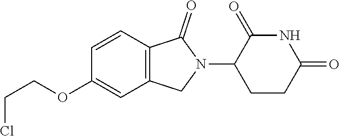 [SMILES: O=C1CCC(N2Cc3cc(OCCCl)ccc3C2=O)C(=O)N1] |
| | | MS |
| | | found |
| Compd | Product | [M + H]+ |
| | 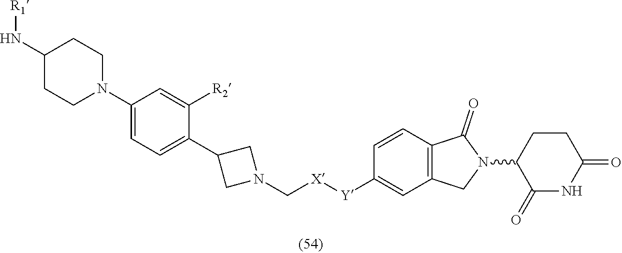 | |
| 23-1 | 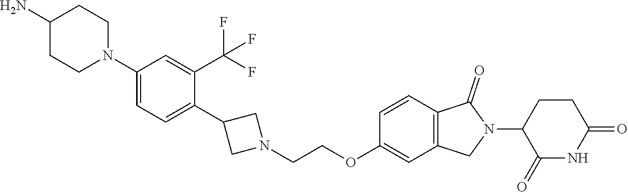 [SMILES: NC1CCN(c2ccc(C3CN(CCOc4ccc5c(c4)CN(C4CCC(=O)NC4=O)C5=O)C3)c(C(F)(F)F)c2)CC1] | 298.8 |
| 23-2 | 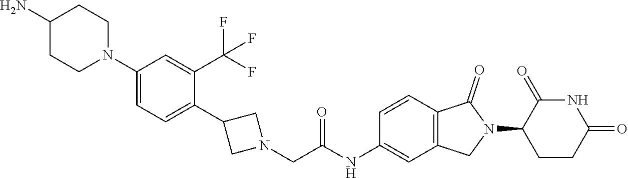 [SMILES: NC1CCN(c2ccc(C3CN(CC(=O)Nc4ccc5c(c4)CN([C@@H]4CCC(=O)NC4=O)C5=O)C3)c(C(F)(F)F)c2)CC1] | 599.4, 300.3 |
| 23-3 | 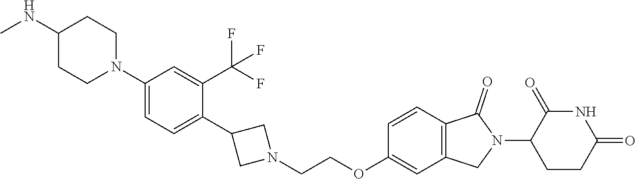 [SMILES: CNC1CCN(c2ccc(C3CN(CCOc4ccc5c(c4)CN(C4CCC(=O)NC4=O)C5=O)C3)c(C(F)(F)F)c2)CC1] | 300 |
| 23-4 | 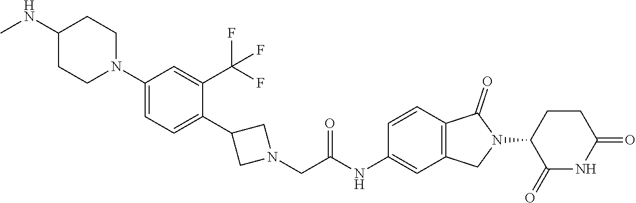 [SMILES: CNC1CCN(c2ccc(C3CN(CC(=O)Nc4ccc5c(c4)CN([C@@H]4CCC(=O)NC4=O)C5=O)C3)c(C(F)(F)F)c2)CC1] | 307.2 |
| 23-5 | 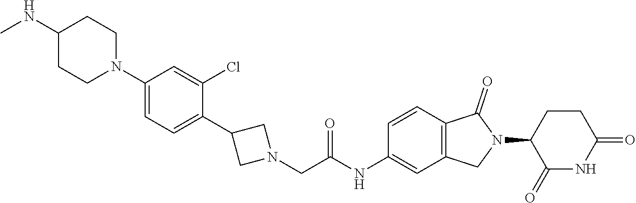 [SMILES: CNC1CCN(c2ccc(C3CN(CC(=O)Nc4ccc5c(c4)CN([C@H]4CCC(=O)NC4=O)C5=O)C3)c(Cl)c2)CC1] | 579.4, 290.3 |
| 23-6 | 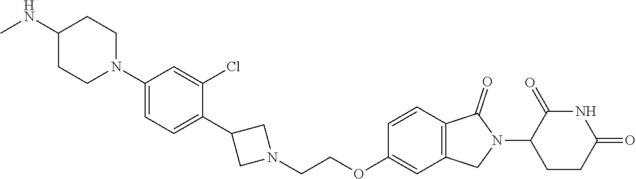 [SMILES: CNC1CCN(c2ccc(C3CN(CCOc4ccc5c(c4)CN(C4CCC(=O)NC4=O)C5=O)C3)c(Cl)c2)CC1] | 566.4, 284.0 |

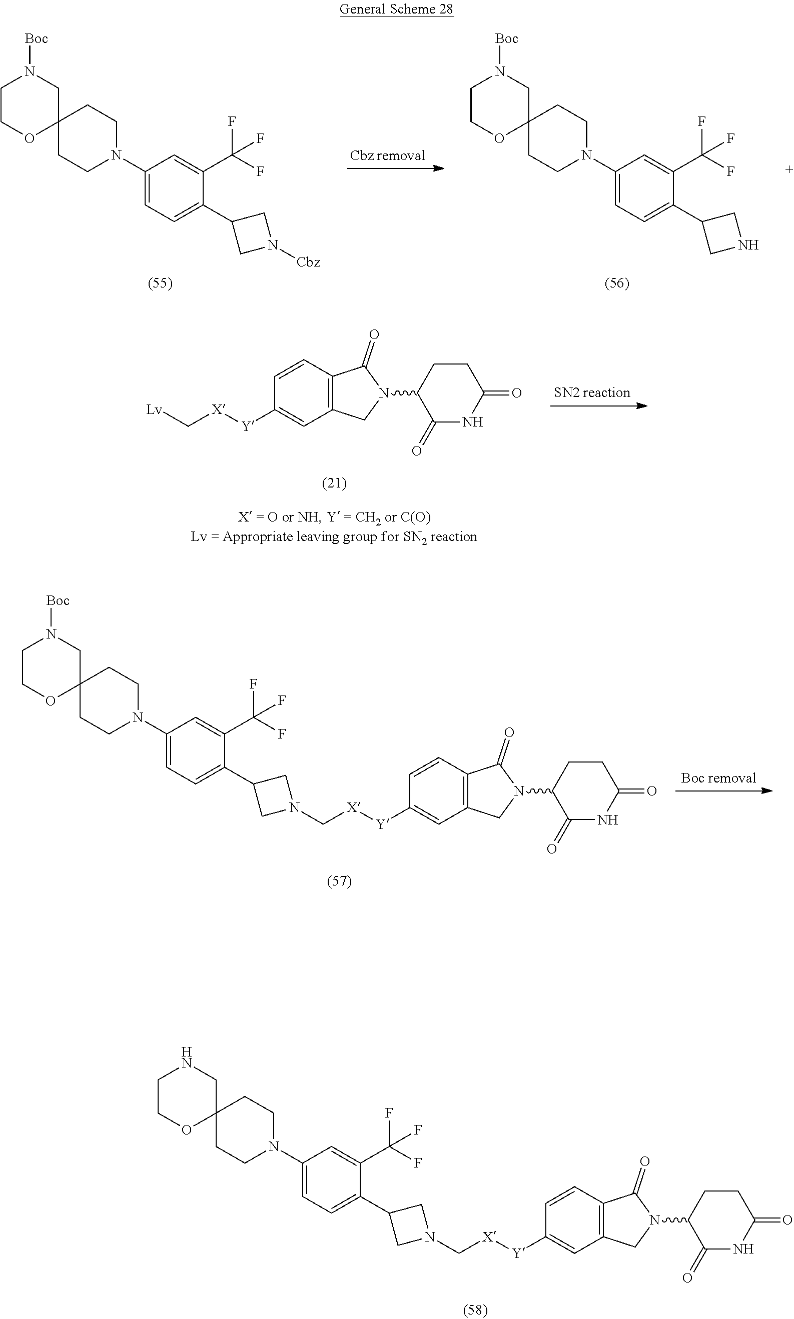

### Method 78

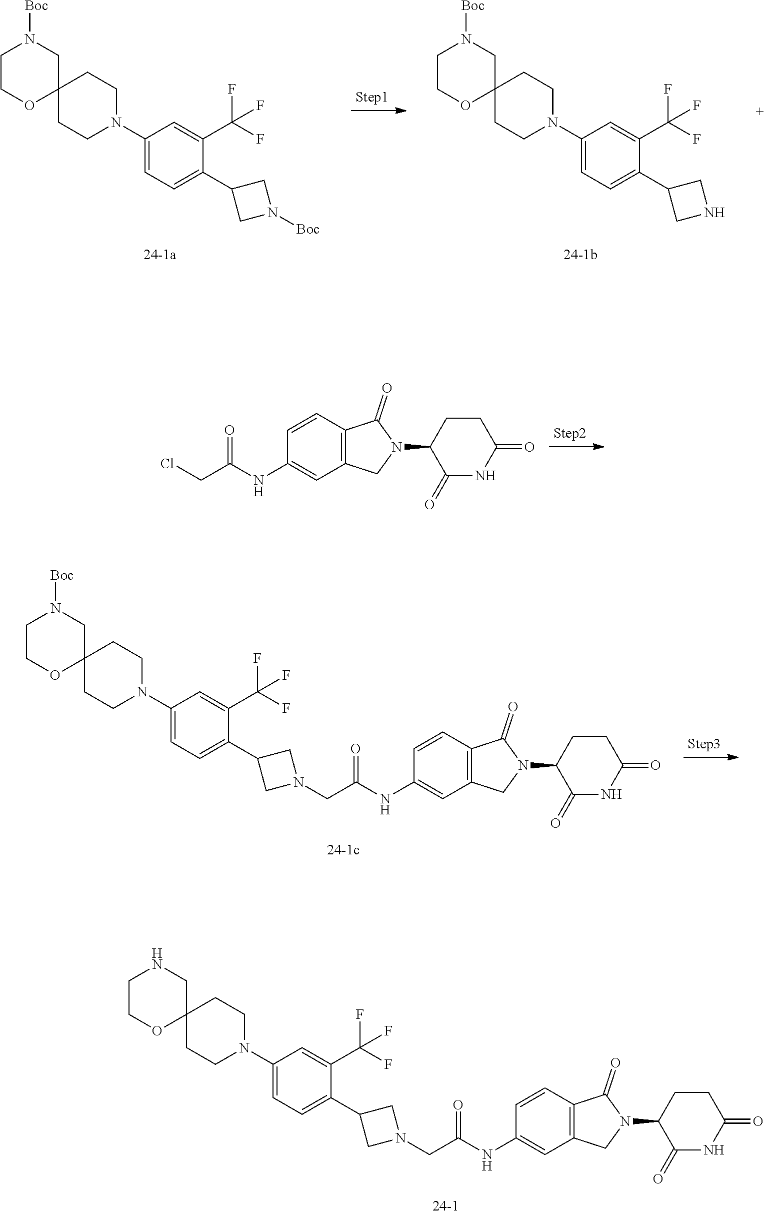 [SMILES: CC(C)(C)OC(=O)N1CC(c2ccc(N3CCC4(CC3)CN(C(=O)OC(C)(C)C)CCO4)cc2C(F)(F)F)C1 ; CC(C)(C)OC(=O)N1CCOC2(CCN(c3ccc(C4CNC4)c(C(F)(F)F)c3)CC2)C1 ; O=C1CC[C@H](N2Cc3cc(NC(=O)CCl)ccc3C2=O)C(=O)N1 ; CC(C)(C)OC(=O)N1CCOC2(CCN(c3ccc(C4CN(CC(=O)Nc5ccc6c(c5)CN([C@H]5CCC(=O)NC5=O)C6=O)C4)c(C(F)(F)F)c3)CC2)C1 ; O=C1CC[C@H](N2Cc3cc(NC(=O)CN4CC(c5ccc(N6CCC7(CC6)CNCCO7)cc5C(F)(F)F)C4)ccc3C2=O)C(=O)N1]

Step 1: A mixture of tert-butyl 9-[4-(1-benzyloxycarbonylazetidin-3-yl)-3-(trifluoromethyl)phenyl]-1-oxa-4,9-diazaspiro[5.5]undecane-4-carboxylate (24-1a, 385 mg, 652.94 μmol) and Pd/C (10% on Carbon (wetted with ca. 55% Water)) (69.5 mg, 652.94 μmol) in MEOH (5 mL) was equipped with a hydrogen gas balloon and stirred for overnight at a room temperature. The mixture was filtered through a cellite, and the filtrated was collected. concentrated, and no further purified to afford tert-butyl 9-(4-(azetidin-3-yl)-3-(trifluoromethyl)phenyl)-1-oxa-4,9-diazaspiro[5.5]undecane-4-carboxylate (24-1b, 295 mg, 647.62 μmol, 99.2% yield). ESI-MS: m/z=456.6 [M+1]⁺.
Step 2: A mixture of 2-chloro-N-[2-[(3S)-2,6-dioxo-3-piperidyl]-1-oxo-isoindolin-5-yl]acetamide (110.56 mg, 329.30 μmol), tert-butyl 9-[4-(azetidin-3-yl)-3-(trifluoromethyl)phenyl]-1-oxa-4,9-diazaspiro[5.5]undecane-4-carboxylate (125 mg, 274.42 μmol), and K₂CO₃ (151.70 mg, 1.10 mmol) in DMF (1 mL) was heated to 80° C. and shaken for 10 hours. The reaction mixture was cooled to a room temperature, then diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford tert-butyl 9-[4-[1-[2-[[2-[(3S)-2,6-dioxo-3-piperidyl]-1-oxo-isoindolin-5-yl]amino]-2-oxo-ethyl]azetidin-3-yl]-3-(trifluoromethyl)phenyl]-1-oxa-4,9-diazaspiro[5.5]undecane-4-carboxylate (160 mg, 211.98 μmol, 77.25% yield) (24-1c). MeOH (5 to 50%) in EtOAc. ESI-MS: m/z=755.2 [M+1]⁺.
Step 3: A solution of tert-butyl 9-[4-[1-[2-[[2-[(3S)-2,6-dioxo-3-piperidyl]-1-oxo-isoindolin-5-yl]amino]-2-oxo-ethyl]azetidin-3-yl]-3-(trifluoromethyl)phenyl]-1-oxa-4,9-diazaspiro[5.5]undecane-4-carboxylate (160 mg, 211.98 μmol) in DCM (2 mL) was treated with TFA (0.5 mL) at a room temperature and shaken for 1 hour. The mixture was concentrated and purified by a flash column to afford N-[2-[(3S)-2,6-dioxo-3-piperidyl]-1-oxo-isoindolin-5-yl]-2-[3-[4-(1-oxa-4,9-diazaspiro[5.5]undecan-9-yl)-2-(trifluoromethyl)phenyl]azetidin-1-yl]acetamide (46 mg, 61.83 μmol, 29.17% yield, 88% purity) (24-1). MeOH (10 to 70%) in EtOAc. ESI-MS: m/z=655.4 [M+1]⁺

The following compounds 24-1 through 24-2 (58) shown in Table 21 were prepared by methods similar to those described for the preparation of 24-1, using the appropriate intermediates (55 and 21).

TABLE 21

| Compd | | |
|---|---|---|
| | 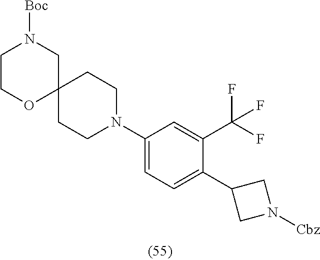 [SMILES: CC(C)(C)OC(=O)N1CCOC2(CCN(c3ccc(C4CN(C(=O)OCc5ccccc5)C4)c(C(F)(F)F)c3)CC2)C1] | 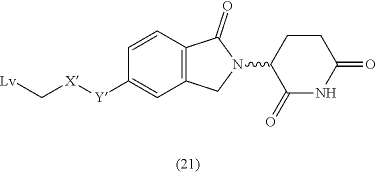 |
| 24-1 | 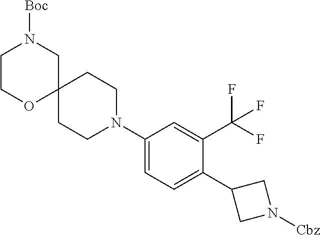 [SMILES: CC(C)(C)OC(=O)N1CCOC2(CCN(c3ccc(C4CN(C(=O)OCc5ccccc5)C4)c(C(F)(F)F)c3)CC2)C1] | 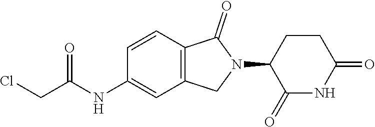 [SMILES: O=C1CC[C@H](N2Cc3cc(NC(=O)CCl)ccc3C2=O)C(=O)N1] |
| 24-2 | 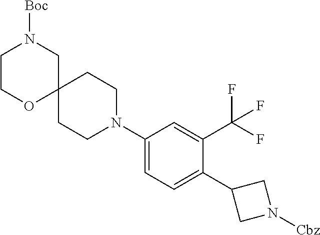 [SMILES: CC(C)(C)OC(=O)N1CCOC2(CCN(c3ccc(C4CN(C(=O)OCc5ccccc5)C4)c(C(F)(F)F)c3)CC2)C1] | 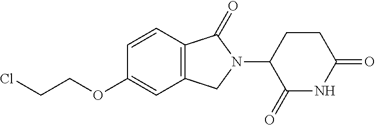 [SMILES: O=C1CCC(N2Cc3cc(OCCCl)ccc3C2=O)C(=O)N1] |
| | | MS |
| | | found |
| Compd | Product | [M + H]+ |
| | 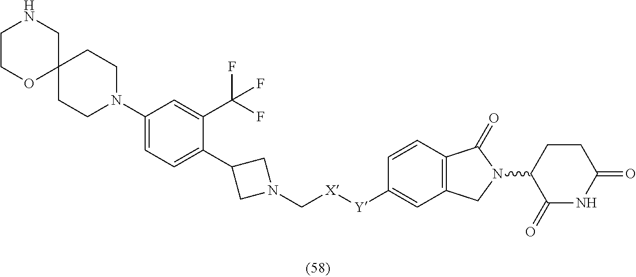 | |
| 24-1 | 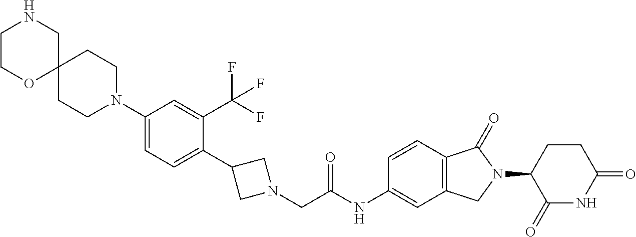 [SMILES: O=C1CC[C@H](N2Cc3cc(NC(=O)CN4CC(c5ccc(N6CCC7(CC6)CNCCO7)cc5C(F)(F)F)C4)ccc3C2=O)C(=O)N1] | 655.4, 328.0 |
| 24-2 | 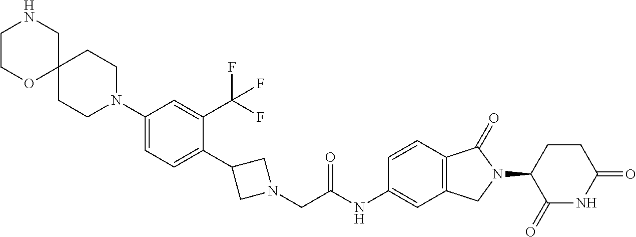 [SMILES: O=C1CC[C@H](N2Cc3cc(NC(=O)CN4CC(c5ccc(N6CCC7(CC6)CNCCO7)cc5C(F)(F)F)C4)ccc3C2=O)C(=O)N1] | 642.5, 321.8 |

### Method 79

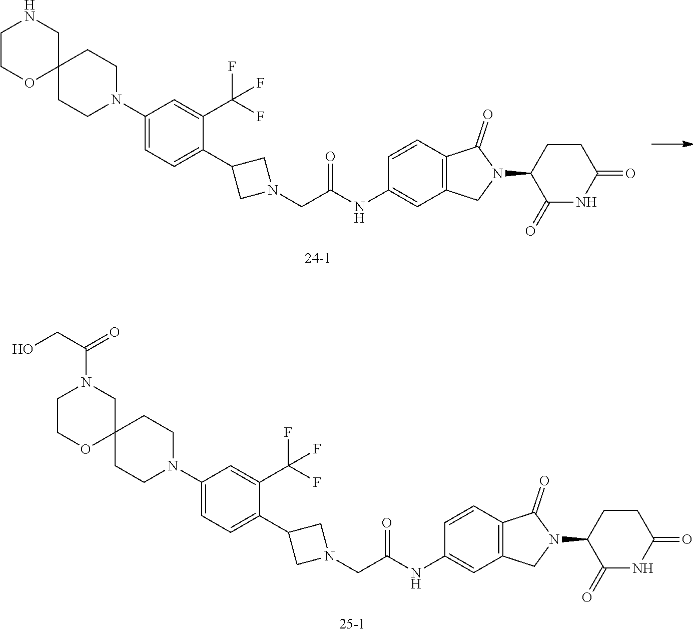 [SMILES: O=C1CC[C@H](N2Cc3cc(NC(=O)CN4CC(c5ccc(N6CCC7(CC6)CNCCO7)cc5C(F)(F)F)C4)ccc3C2=O)C(=O)N1 ; O=C1CC[C@H](N2Cc3cc(NC(=O)CN4CC(c5ccc(N6CCC7(CC6)CN(C(=O)CO)CCO7)cc5C(F)(F)F)C4)ccc3C2=O)C(=O)N1]

A mixture of 2-hydroxyacetic acid (3.08 mg, 40.52 μmol, 2.43 μL), 3-[5-[2-[3-[4-(1-oxa-4, 9-diazaspiro[5.5]undecan-9-yl)-2-(trifluoromethyl)phenyl]azetidin-1-yl]ethoxy]-1-oxo-isoindolin-2-yl]piperidine-2,6-dione (24-1, 13 mg, 20.26 μmol), HATU (11.6 mg, 30.39 μmol), and DIPEA (7.86 mg, 60.78 μmol, 10.62 μL) in DMF (1 mL) was shaken for 6 hours at a room temperature. The reaction mixture was diluted with EtOAc (˜10 mL) and aq. sat. NaHCO₃ solution (˜10 mL), and then the aqueous phase was separated and extracted with EtOAc (˜3 mL). The organic phases were combined, dried over Na₂SO₄, filtered, concentrated, and purified by a flash column to afford 3-[5-[2-[3-[4-[4-(2-hydroxyacetyl)-1-oxa-4,9-diazaspiro[5.5]undecan-9-yl]-2-(trifluoromethyl)phenyl]azetidin-1-yl]ethoxy]-1-oxo-isoindolin-2-yl]piperidine-2,6-dione (10.5 mg, 14.26 μmol, 70.4% yield, 95% purity) (25-1). MeOH (5 to 40%) in EtOAc. ESI-MS: m/z=700.4 [M+1]⁺.

### Method 80

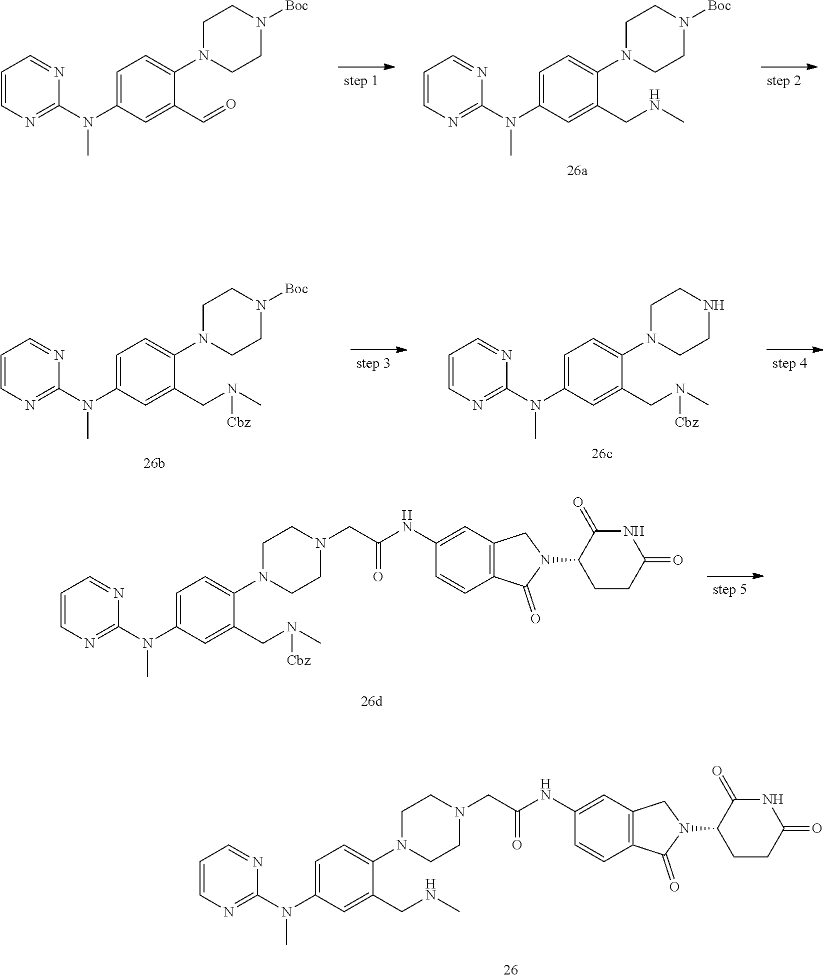 [SMILES: CN(c1ccc(N2CCN(C(=O)OC(C)(C)C)CC2)c(C=O)c1)c1ncccn1 ; CNCc1cc(N(C)c2ncccn2)ccc1N1CCN(C(=O)OC(C)(C)C)CC1 ; CN(Cc1cc(N(C)c2ncccn2)ccc1N1CCN(C(=O)OC(C)(C)C)CC1)C(=O)OCc1ccccc1 ; CN(Cc1cc(N(C)c2ncccn2)ccc1N1CCNCC1)C(=O)OCc1ccccc1 ; CN(Cc1cc(N(C)c2ncccn2)ccc1N1CCN(CC(=O)Nc2ccc3c(c2)CN([C@H]2CCC(=O)NC2=O)C3=O)CC1)C(=O)OCc1ccccc1 ; CNCc1cc(N(C)c2ncccn2)ccc1N1CCN(CC(=O)Nc2ccc3c(c2)CN([C@H]2CCC(=O)NC2=O)C3=O)CC1]

Step 1: To a stirred solution of tert-butyl 4-(2-formyl-4-(methyl(pyrimidin-2-yl)amino)phenyl)piperazine-1-carboxylate (400.0 mg, 1.00 mmol) in MeOH (5 mL) was added methylamine (62.4 mg, 2.01 μmol), AcOH (0.2 mL) and NaBH₃CN (189.2 mg, 3.01 mmol). The reaction was stirred 25° C. for 8 hours. The reaction mixture was concentrated to afford a crude product. The crude product was purified by Prep-TLC (DCM/MeOH=20/1) to give tert-butyl 4-(4-(methyl(pyrimidin-2-yl)amino)-2-((methylamino)methyl)phenyl)piperazine-1-carboxylate (26a, 260.0 mg, 62.63% yield) as a yellow solid. ESI-MS: m/z=413.1 [M+1]⁺.
Step 2: A solution of tert-butyl 4-(4-(methyl(pyrimidin-2-yl)amino)-2-((methylamino)methyl)phenyl)piperazine-1-carboxylate (200.0 mg, 0.48 mmol), CbzCl (99.0 mg, 0.58 mmol) and TEA (97.9 mg, 0.97 mmol) in THF (5 mL) was stirred at 25° C. for 12 hours. The mixture was filtered. The filtrate was concentrated to afford a crude product. The crude product was purified by silica gel chromatography (PE/EtOAc=20/1˜5/1) to give tert-butyl 4-(2-((((benzyloxy)carbonyl)(methyl)amino)methyl)-4-(methyl(pyrimidin-2-yl)amino)phenyl)piperazine-1-carboxylate (26b, 180 mg, 87.64% yield) as a yellow solid. ESI-MS: m/z=547.2 [M+1]⁺.
Step 3: A solution of tert-butyl 4-(2-((((benzyloxy)carbonyl)(methyl)amino)methyl)-4-(methyl(pyrimidin-2-yl)amino)phenyl)piperazine-1-carboxylate (180.0 mg, 0.33 mmol), TEA (187.4 mg, 1.65 mmol) in DCM (5 mL) was stirred at 25° C. for 12 hours. The mixture was concentrated to give benzyl methyl(5-(methyl(pyrimidin-2-yl)amino)-2-(piperazin-1-yl)benzyl)carbamate (26c, 120 mg, 81.61% yield). use directly without purification. ESI-MS: m/z=447.1 [M+1]⁺.
Step 4: A solution of benzyl methyl(5-(methyl(pyrimidin-2-yl)amino)-2-(piperazin-1-yl)benzyl)carbamate (120.0 mg, 0.27 mol), (S)-2-chloro-N-(2-(2,6-dioxopiperidin-3-yl)-1-oxoisoindolin-5-yl)acetamide (90.2 mg, 0.27 mmol) and TEA (104.2 mg, 0.81 mmol) in DMSO (5 mL) was stirred at 85° C. for 3 hours. The reaction mixture was poured into water (20 mL) and extracted with EtOAc (20 mL×3). The combined organic layers were washed with brine, dried over Na₂SO₄ and concentrated to afford a crude product. The residue was purified by Prep-HPLC and lyophilized to give benzyl (S)-(2-(4-(2-((2-(2,6-dioxopiperidin-3-yl)-1-oxoisoindolin-5-yl)amino)-2-oxoethyl)piperazin-1-yl)-5-(methyl(pyrimidin-2-yl)amino)benzyl)(methyl)carbamate (26d, 45.0 mg, 22.45% yield) as a yellow solid. ¹H NMR (400 MHz, DMSO d₆) δ 1.97-2.04 (m, 1H), 2.32-2.41 (m, 1H), 2.56-2.65 (m, 2H), 2.85 (s, 3H), 2.88-2.97 (m, 1H), 3.16 (br. s, 4H), 3.31-3.43 (m, 4H), 3.55-3.64 (m, 3H), 4.33 (d, J=17.6 Hz, 3H), 4.47 (d, J=17.6 Hz, 1H), 4.57 (s, 2H), 5.02-5.14 (m, 3H), 6.74 (t, J=4.8 Hz, 1H), 6.97-7.04 (m, 1H), 7.18-7.35 (m, 7H), 7.67 (d, J=9.6 Hz, 1H), 7.75 (d, J=8.4 Hz, 1H), 7.95 (s, 1H), 8.34 (d, J=4.8 Hz, 2H), 10.26 (br. s, 1H), 10.90-11.07 (d, J=19.2 Hz, 2H). ESI-MS: m/z=746.3 [M+1]⁺.
Step 5: A solution of benzyl (S)-(2-(4-(2-((2-(2,6-dioxopiperidin-3-yl)-1-oxoisoindolin-5-yl)amino)-2-oxoethyl)piperazin-1-yl)-5-(methyl(pyrimidin-2-yl)amino)benzyl)(methyl)carbamate (135.0 mg, 0.18 mmol) in HCl in EtOAc (2.5 mol/L, 10 mL) was stirred at 25° C. for 12 hours. The mixture was concentrated to afford a crude product. The crude product was purified by Prep-TLC (DCM/MeOH=10/1) to give (S)—N-(2-(2,6-dioxopiperidin-3-yl)-1-oxoisoindolin-5-yl)-2-(4-(4-(methyl(pyrimidin-2-yl)amino)-2-((methylamino)methyl)phenyl)piperazin-1-yl)acetamide (26, 21.0 mg, 18.97% yield) as a white solid. ¹H NMR (400 MHz, DMSO_d₆) δ 1.96-2.05 (m, 1H), 2.33-2.35 (m, 1H), 2.47 (s, 3H), 2.57-2.63 (m, 1H), 2.74 (s, 4H), 2.86-2.96 (m, 5H), 3.29 (s, 2H), 3.43 (s, 3H), 3.97 (s, 2H), 4.30 (d, J=17.4 Hz, 1H), 4.44 (d, J=17.4 Hz, 1H), 5.09 (dd, J=13.2, 5.1 Hz, 1H), 6.72 (t, J=4.7 Hz, 1H), 7.21-7.31 (m, 2H), 7.41 (s, 1H), 7.66-7.75 (m, 2H), 8.04 (s, 1H), 8.26 (s, 1H), 8.35 (d, J=4.7 Hz, 2H), 10.18 (s, 1H), 10.96 (br. s, 1H). ESI-MS: m/z=612.1 [M+1]⁺.

### Method 81

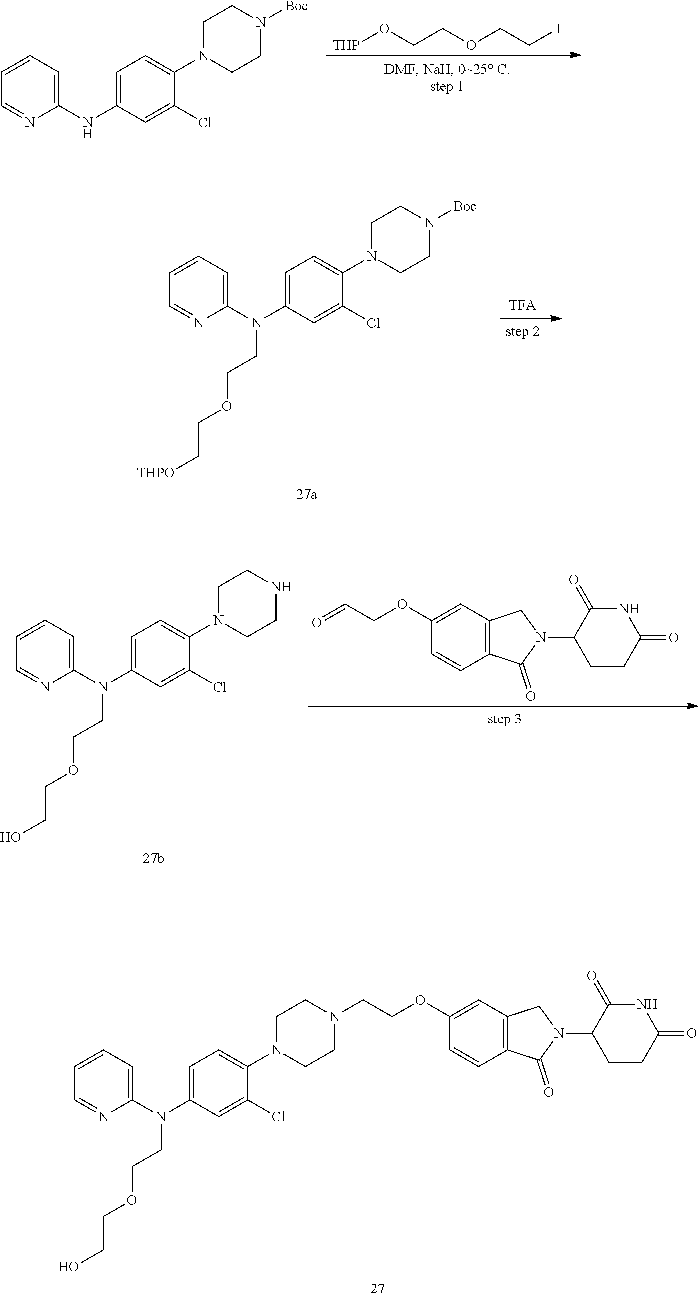

Step 1: To a stirred solution of tert-butyl 4-(2-chloro-4-(pyridin-2-ylamino)phenyl)piperazine-1-carboxylate (6.0 g, 15.43 mmol) in DMF (100 mL) was added NaH (3.09 g, 77.15 mmol) dropwise at 0° C. The mixture was stirred at 0° C. for 3 hours. Then 2-(2-(2-iodoethoxy)ethoxy)tetrahydro-2H-pyran (4.63 g, 15.43 mol) was slowly added dropwise. The reaction was stirred at 25° C. for 12 hours. The reaction mixture was poured into water (200 mL) and extracted with EtOAc (3×50 mL). The combined organic layers were washed with brine, dried over Na₂SO₄ and concentrated to afford a crude product. The crude product was purified by prep-TLC (DCM/MeOH=10/1) to give tert-butyl 4-(2-chloro-4-(pyridin-2-yl(2-(2-((tetrahydro-2H-pyran-2-yl)oxy)ethoxy)ethyl)amino)phenyl)piperazine-1-carboxylate (27a, 0.80 g, yield 9.24%) as a yellow oil. ESI-MS: m/z=561.1 [M+1]⁺.
Step 2: A solution of tert-butyl 4-(2-chloro-4-(pyridin-2-yl(2-(2-((tetrahydro-2H-pyran-2-yl)oxy)ethoxy)ethyl)amino)phenyl)piperazine-1-carboxylate (800 mg, 1.42 mmol), TFA (487 mg, 4.27 mmol) in DCM (10 mL) was stirred at 25° C. for 12 hours. The mixture was filtered. The filtrate was concentrated to afford a crude product. The residue was purified by prep-HPLC and lyophilized to give 2-(2-((3-chloro-4-(piperazin-1-yl)phenyl)(pyridin-2-yl)amino)ethoxy)ethan-1-ol (27b, 60.0 mg, yield 11.17%) as a colorless oil. ¹H NMR (400 MHz, DMSO_d₆) δ 2.81-2.93 (m, 8H), 3.38 (d, J=5.2 Hz, 3H), 3.42-3.46 (m, 2H), 3.59 (t, J=6.0 Hz, 2H), 3.99 (t, J=6.0 Hz, 2H), 4.56 (s, 1H), 6.34 (d, J=8.4 Hz, 1H), 6.62-6.68 (m, 1H), 7.17 (d, J=8.4 Hz, 1H), 7.24 (dd, J=8.4, 2.4 Hz, 1H), 7.34-7.46 (m, 2H), 8.13 (d, J=4.8 Hz, 1H).
Step 3: A solution of 2-(2-((3-chloro-4-(piperazin-1-yl)phenyl)(pyridin-2-yl)amino)ethoxy)ethan-1-ol (60 mg, 0.16 mmol), 2-((2-(2,6-dioxopiperidin-3-yl)-1-oxoisoindolin-5-yl)oxy)acetaldehyde (48 mg, 0.16 mmol) AcOH (0.2 mL) and NaBH₃CN (30.0 mg, 0.48 mmol) in MeOH (2 mL) was stirred at 25° C. for 12 hours. The mixture was filtered. The filtrate was concentrated to afford a crude product. The crude product was purified by prep-TLC (DCM/MeOH=5/1) to give 3-(5-(2-(4-(2-chloro-4-((2-(2-hydroxyethoxy)ethyl)(pyridin-2-yl)amino)phenyl)piperazin-1-yl)ethoxy)-1-oxoisoindolin-2-yl)piperidine-2,6-dione (27, 65.0 mg, yield 61.57%) as a white solid. ¹H NMR (400 MHz, DMSO_d₆) δ 1.96-2.02 (m, 1H), 2.36-2.41 (m, 1H), 2.56-2.63 (m, 2H), 2.70 (s, 2H), 2.83 (t, J=5.6 Hz, 1H), 2.90-2.96 (m, 1H), 3.03 (s, 2H), 3.34-3.40 (m, 4H), 3.42-3.47 (m, 4H), 3.59 (t, J=6.0 Hz, 1H), 3.99 (t, J=6.0 Hz, 1H), 4.20-4.31 (m, 4H), 4.40 (d, J=17.2 Hz, 1H), 5.04-5.10 (m, 1H), 6.34 (d, J=8.8 Hz, 1H), 6.65 (dd, J=6.8, 5.2 Hz, 1H), 7.09 (dd, J=8.4, 2.0 Hz, 1H), 7.16-7.28 (m, 2H), 7.37-7.43 (m, 2H), 7.62-7.65 (m, 1H), 8.13 (d, J=3.6 Hz, 1H), 10.97 (s, 1H). ESI-MS: m/z=663.2 [M+1]⁺.

### Method 82

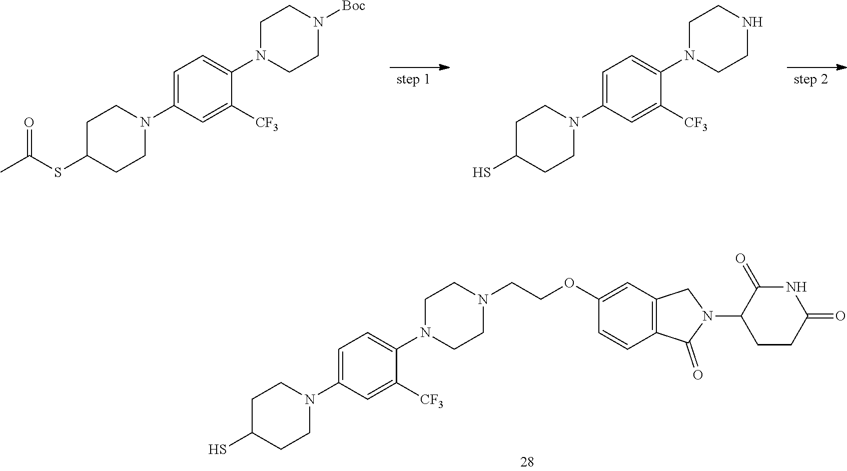 [SMILES: CC(=O)SC1CCN(c2ccc(N3CCN(C(=O)OC(C)(C)C)CC3)c(C(F)(F)F)c2)CC1 ; FC(F)(F)c1cc(N2CCC(S)CC2)ccc1N1CCNCC1 ; O=C1CCC(N2Cc3cc(OCCN4CCN(c5ccc(N6CCC(S)CC6)cc5C(F)(F)F)CC4)ccc3C2=O)C(=O)N1]

Step 1: A solution of tert-butyl 4-(4-(4-(acetylthio)piperidin-1-yl)-2-(trifluoromethyl)phenyl)piperazine-1-carboxylate (0.50 g, 1.03 mmol) and 12 N HCl (2 mL) in 1,4-dioxane (5 mL) was stirred at 100° C. for 12 hours. The mixture was concentrated to give 1-(4-(piperazin-1-yl)-3-(trifluoromethyl)phenyl)piperidine-4-thiol (0.32 g, yield 90.34%) as a red oil, which was used directly without purification. ESI-MS: m/z=346.1 [M+1]⁺.
Step 2: A solution of 1-(4-(piperazin-1-yl)-3-(trifluoromethyl)phenyl)piperidine-4-thiol (100 mg, 0.29 mmol), 2-((2-(2,6-dioxopiperidin-3-yl)-1-oxoisoindolin-5-yl)oxy)acetaldehyde (87 mg, 0.29 mmol) and NaBH₃CN (55 mg, 0.87 mmol) in MeOH (2 mL) was stirred at 25° C. for 6 hours. The mixture was concentrated to afford a crude product. The residue was purified by prep-HPLC and lyophilized to give 3-(5-(2-(4-(4-(4-mercaptopiperidin-1-yl)-2-(trifluoromethyl)phenyl)piperazin-1-yl)ethoxy)-1-oxoisoindolin-2-yl)piperidine-2,6-dione (28, 20.0 mg, yield 10.94%) as a white solid. ¹H NMR (400 MHz, DMSO_d₆) δ 1.51-1.64 (m, 2H), 1.96-2.01 (m, 2H), 2.34-2.42 (m, 1H), 2.56-2.64 (m, 1H), 2.68 (d, J=6.8 Hz, 1H), 2.80-2.90 (m, 2H), 2.89-3.07 (m, 2H), 3.06-3.18 (m, 2H), 3.24 (s, 2H), 3.58-3.70 (m, 4H), 3.70-3.77 (m, 2H), 4.30 (d, J=17.2 Hz, 1H), 4.39-4.50 (m, 3H), 5.09 (dd, J=13.2, 5.2 Hz, 1H), 7.08-7.18 (m, 2H), 7.20-7.29 (m, 2H), 7.40 (d, J=8.8 Hz, 1H), 7.70 (d, J=8.4 Hz, 1H), 9.88 (s, 1H), 10.98 (s, 1H). ESI-MS: m/z=632.3 [M+1]⁺

### Method 83

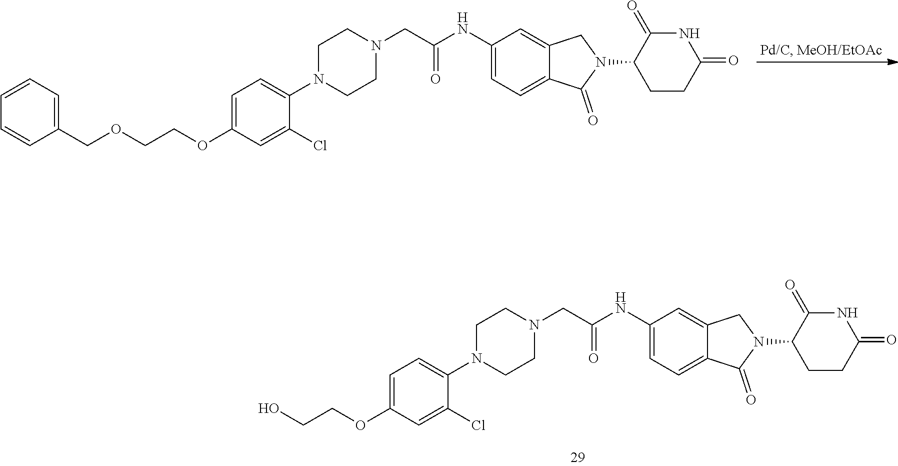 [SMILES: O=C1CC[C@H](N2Cc3cc(NC(=O)CN4CCN(c5ccc(OCCOCc6ccccc6)cc5Cl)CC4)ccc3C2=O)C(=O)N1 ; O=C1CC[C@H](N2Cc3cc(NC(=O)CN4CCN(c5ccc(OCCO)cc5Cl)CC4)ccc3C2=O)C(=O)N1]

A solution of 2-[4-[4-(2-benzyloxyethoxy)-2-chloro-phenyl]piperazin-1-yl]-N-[2-[(3S)-2,6-dioxo-3-piperidyl]-1-oxo-isoindolin-5-yl]acetamide (150 mg, 232.15 μmmol), 10% Pd/C (24.71 mg) in MeOH/EtOAc (1/1, 10 mL) was stirred at 25° C. for overnight under of H₂. The mixture was filtered, and the filtrate was concentrated to afford a crude product. The crude product was purified by silica gel chromatography (DCM/MeOH=10:1) to obtain title compound (29, 14.6 mg, 11.2% yield). ESI-MS: m/z=556.4, 558.4 [M+1]⁺.

### Method 84

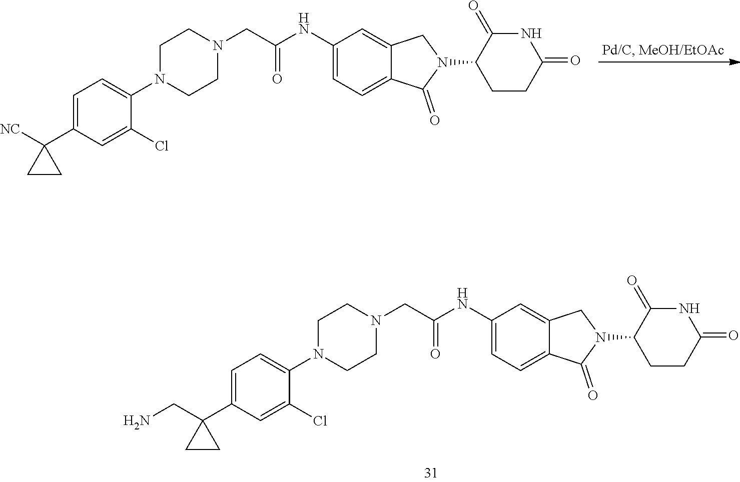 [SMILES: N#CC1(c2ccc(N3CCN(CC(=O)Nc4ccc5c(c4)CN([C@H]4CCC(=O)NC4=O)C5=O)CC3)c(Cl)c2)CC1 ; NCC1(c2ccc(N3CCN(CC(=O)Nc4ccc5c(c4)CN([C@H]4CCC(=O)NC4=O)C5=O)CC3)c(Cl)c2)CC1]

A solution of 2-[4-[2-chloro-4-(1-cyanocyclopropyl)phenyl]piperazin-1-yl]-N-[2-[(3S)-2,6-dioxo-3-piperidyl]-1-oxo-isoindolin-5-yl]acetamide (100 mg, 178.24 μmmol), 10% Pd/C (18.97 mg, 178.24 μmol) in MeOH/EtOAc (1/1, 10 mL) was stirred at 25° C. for overnight under of H₂. The mixture was filtered, and the filtrate was concentrated to afford a crude product. The crude product was purified by silica gel chromatography (DCM/MeOH=10:1 to 5:1) to obtain title compound (31, 32.7 mg, 30.7% yield). ESI-MS: m/z=565.4, 567.4 [M+1]⁺.

### Method 85

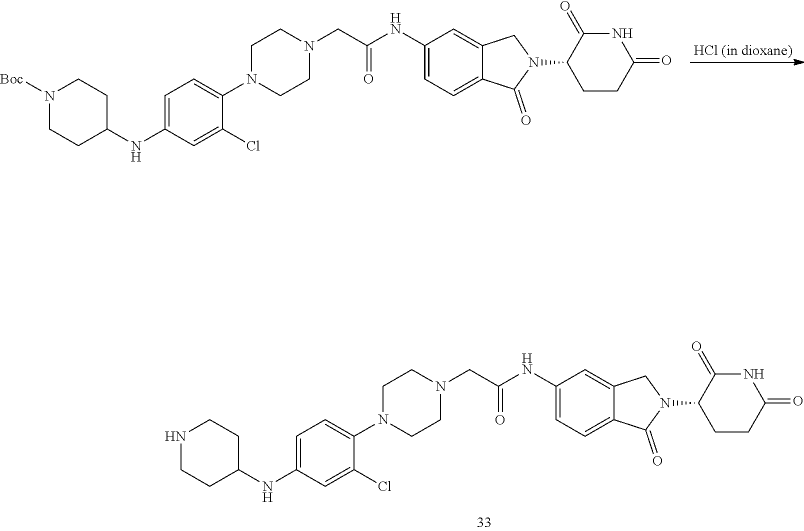 [SMILES: CC(C)(C)OC(=O)N1CCC(Nc2ccc(N3CCN(CC(=O)Nc4ccc5c(c4)CN([C@H]4CCC(=O)NC4=O)C5=O)CC3)c(Cl)c2)CC1 ; O=C1CC[C@H](N2Cc3cc(NC(=O)CN4CCN(c5ccc(NC6CCNCC6)cc5Cl)CC4)ccc3C2=O)C(=O)N1]

To a solution of tert-butyl tert-butyl 4-[3-chloro-4-[4-[2-[[2-[(3S)-2,6-dioxo-3-piperidyl]-1-oxo-isoindolin-5-yl]amino]-2-oxo-ethyl]piperazin-1-yl]anilino]piperidine-1-carboxylate (100 mg, 144.05 μmmol) in HCl (105.04 μL, 4.0 N in 1,4-dioxane). The reaction mixture was stirred at room temperature for 2 hours. The reaction mixture was concentrated to afford a crude product. The crude product was purified by silica gel chromatography (DCM/MeOH=10:1 to 3:7) to give 2-[4-[2-chloro-4-(4-piperidylamino)phenyl]piperazin-1-yl]-N-[2-[(3S)-2,6-dioxo-3-piperidyl]-1-oxo-isoindolin-5-yl]acetamide (33, 52 mg, 56.6% yield). ESI-MS: m/z=594.4, 596.5 [M+1]⁺.

### Method 86

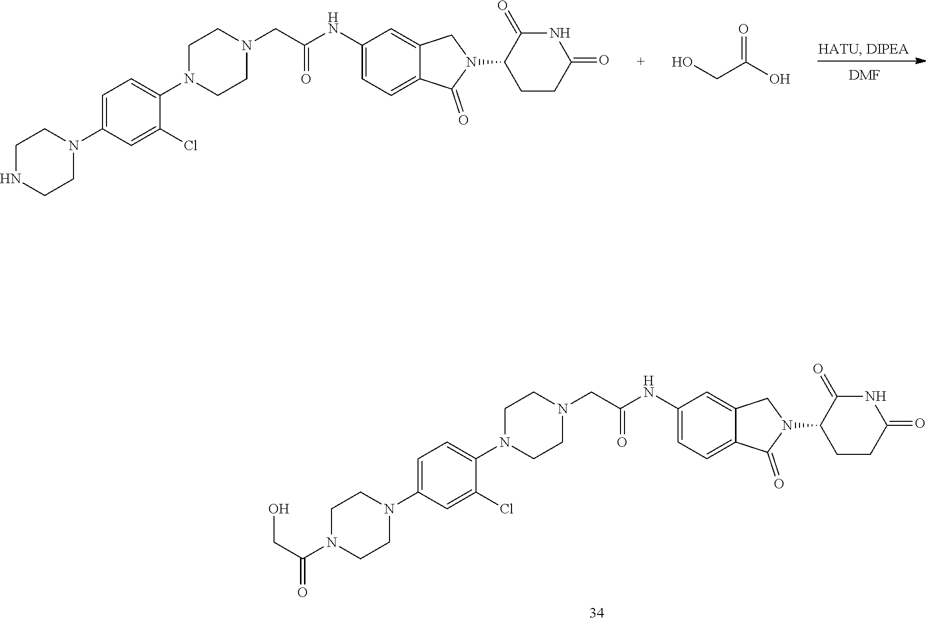

To a solution of 2-[4-(2-chloro-4-piperazin-1-yl-phenyl)piperazin-1-yl]-N-[2-[(3S)-2,6-dioxo-3-piperidyl]-1-oxo-isoindolin-5-yl]acetamide (30 mg, 51.72 μmol), 2-hydroxyacetic acid (5.90 mg, 77.58 μmol) in DMF (1 mL) was added HATU (39.33 mg, 103.43 μmol), DIPEA (26.74 mg, 206.87 μmol) at room temperature for 1 hour. The reaction mixture was then concentrated in vacuo, and purified by silica gel chromatography (DCM/MeOH=30:1 to 15:1) to give 2-[4-[2-chloro-4-[4-(2-hydroxyacetyl)-piperazin-1-yl]phenyl]piperazin-1-yl]-N-[2-[(3S)-2,6-dioxo-3-piperidyl]-1-oxo-isoindolin-5-yl]acetamide (34, 15.7 mg, 44.0% yield). ESI-MS: m/z=638.4, 640.3 [M+1]⁺.

### Method 87

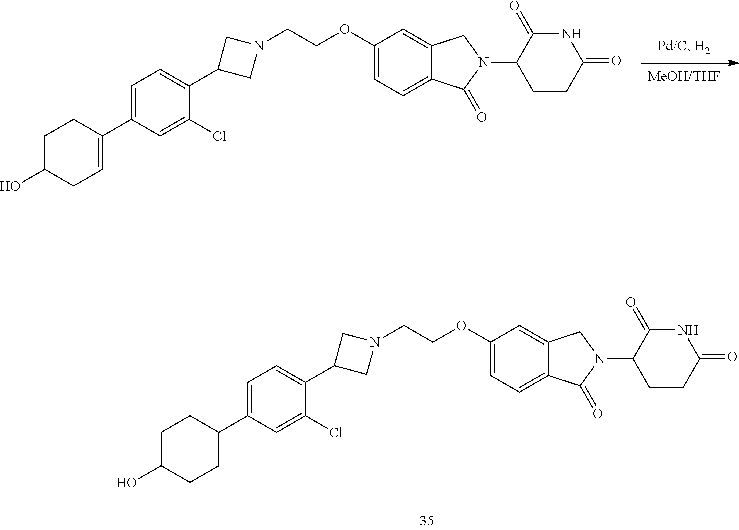 [SMILES: O=C1CCC(N2Cc3cc(OCCN4CC(c5ccc(C6=CCC(O)CC6)cc5Cl)C4)ccc3C2=O)C(=O)N1 ; O=C1CCC(N2Cc3cc(OCCN4CC(c5ccc(C6CCC(O)CC6)cc5Cl)C4)ccc3C2=O)C(=O)N1]

A solution of 3-[5-[2-[3-[2-chloro-4-(4-hydroxycyclohexen-1-yl)phenyl]azetidin-1-yl]ethoxy]-1-oxo-isoindolin-2-yl]piper4idine-2,6-dione (50 mg, 90.90 μmmol), 10% Pd/C (9.67 mg, 90.90 μmol) in MeOH/THF (1:1, 8 mL) was stirred at 25° C. for overnight under of H₂. The mixture was filtered, and the filtrate was concentrated to afford a crude product. The crude product was purified by silica gel chromatography (DCM/MeOH=30:1 to 10:1) to obtain title compound (35, 13.9 mg, 27.1% yield). ESI-MS: m/z=552.3, 554.3 [M+1]⁺.

### Method 88

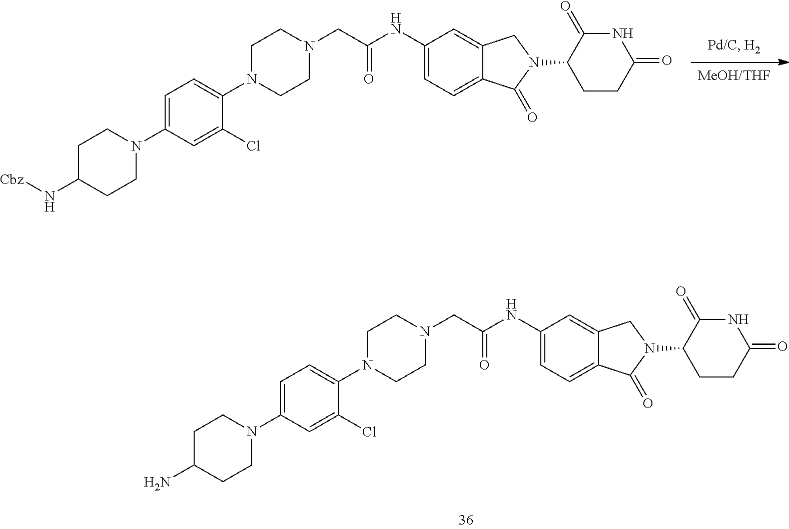 [SMILES: O=C1CC[C@H](N2Cc3cc(NC(=O)CN4CCN(c5ccc(N6CCC(NC(=O)OCc7ccccc7)CC6)cc5Cl)CC4)ccc3C2=O)C(=O)N1 ; NC1CCN(c2ccc(N3CCN(CC(=O)Nc4ccc5c(c4)CN([C@H]4CCC(=O)NC4=O)C5=O)CC3)c(Cl)c2)CC1]

A solution of benzyl N-[1-[3-chloro-4-[4-[2-[[2-[(3R)-2,6-dioxo-3-piperidyl]-1-oxo-isoindolin-5-yl]amino]-2-oxo-ethyl]piperazin-1-yl]phenyl]-4-piperidyl]carbamate (130 mg, 178.51 μmmol), 10% Pd/C (4.75 mg, 44.63 μmol) in MeOH/THF (1/1, 6 mL) was stirred at 25° C. for overnight under of H₂. The mixture was filtered, and the filtrate was concentrated to afford a crude product. The crude product was purified by silica gel chromatography (EtOAc/2% TEA in MeOH=90/10 to 75/25) to obtain title compound (36, 6.9 mg, 6.23% yield). ESI-MS: m/z=594.3, 596.3 [M+1].

### Method 89

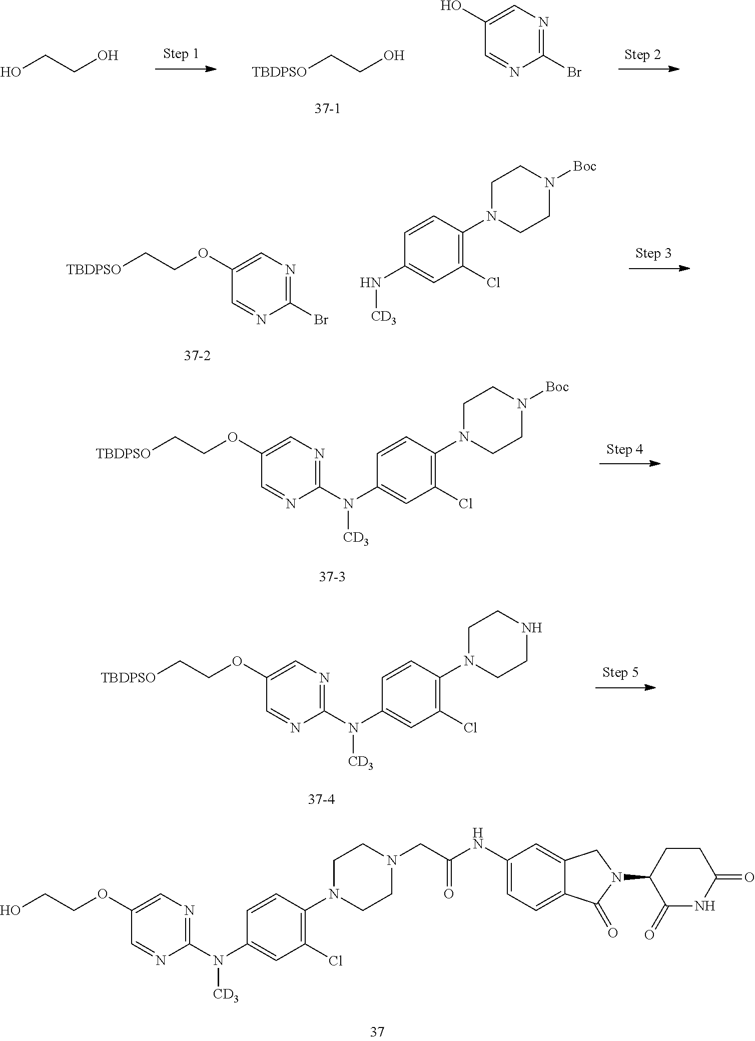

Step 1: To a solution of ethylene glycol (1.97 mL, 35.3 mmol) and in pyridine (2.0 mL, 25 mmol) was added tert-butyl-chloro-diphenyl-silane (1.0 mL, 3.9 mmol). The mixture was stirred at room temperature for 1 hour, poured over H₂O, and extracted with EtOAc. The combined organic layers were washed with brine, dried over Na₂SO₄, filtered, and concentrated. The residue was purified by FCC on SiO₂ using a gradient of 10-50% EtOAc in heptanes. 37-1 was collected as a colorless oil in 60% yield (692 mg, 2.30 mmol). TLC (10% EtOAc/heptanes): R_{f}=0.14, TLC (30% EtOAc/heptanes): R_{f}=0.46. LC/MS found 323.3 [M+Na]⁺. ¹H NMR (400 MHz, CHLOROFORM-d) δ ppm 7.67 (dd, J=7.8, 1.5 Hz, 4H), 7.41 (m, 6H), 3.77 (m, 2H), 3.69 (m, 2H), 2.11 (t, J=6.4 Hz, 1H), 1.07 (s, 9H).
Step 2: To a mixture of 2-[tert-butyl(diphenyl)silyl]oxyethanol (692 mg, 2.30 mmol), 2-bromopyrimidin-5-ol (442 mg, 2.53 mmol), and triphenylphosphine (670 mg, 2.55 mmol) in toluene (13 mL) was added bis(2-methoxyethyl) azodicarboxylate (594 mg, 2.54 mmol). The mixture was degassed via sparging with N₂ for 5 minutes and heated to 100° C. while stirring overnight. The mixture was poured over H₂O and extracted with EtOAc. The combined organic layers were washed with 1N aq. NaOH solution (2 times), washed with H₂O, washed with brine, dried over Na₂SO₄, filtered, and concentrated. The residue was purified by FCC on SiO₂ using a gradient of 5-30% EtOAc in heptanes. 37-2 was collected as a viscous colorless oil in 93% yield (978 mg, 2.14 mmol). TLC (10% EtOAc/heptanes): R_{f}=0.13, TLC (30% EtOAc/heptanes): R_{f}=0.49. LC/MS found 456.9 [M+H]⁺. ¹H NMR (500 MHz, CHLOROFORM-d) δ ppm 8.19 (s, 2H), 7.65 (m, 4H), 7.44 (t, J=7.5 Hz, 2H), 7.38 (d, J=7.3 Hz, 4H), 4.15 (t, J=4.6 Hz, 2H), 4.01 (t, J=4.7 Hz, 2H), 1.05 (s, 9H).
Step 3: A mixture of 2-(2-bromopyrimidin-5-yl)oxyethoxy-tert-butyl-diphenyl-silane (456 mg, 1.0 mmol), tert-butyl 4-[2-chloro-4-(trideuteriomethylamino)phenyl]piperazine-1-carboxylate (329 mg, 1.00 mmol), methanesulfonato(2-dicyclohexylphosphino-2′,6′-di-i-propoxy-1,1′-biphenyl)(2′-methylamino-1,1′-biphenyl-2-yl)palladium(II) (41 mg, 48 μmol), 2-dicyclohexylphosphino-2′,6′-di-i-propoxy-1,1′-biphenyl (23 mg, 49 μmol), and sodium tert-butoxide (192 mg, 1.99 mmol) in dry 1,4-dioxane (5 mL) was degassed via sparging with N₂ for 10 minutes then heated to 100° C. with stirring overnight. The mixture was cooled to room temperature and filtered through a pad of diatomaceous earth, and the filtrate was concentrated to dryness. The residue was purified by flash column chromatography using a gradient of 50-100% EtOAc in hexanes. 37-3 was collected as an off-white semi-solid in 18% yield (126 mg, 0.18 mmol). TLC (30% EtOAc/hexanes): R_{f}=0.34. LC/MS D.N.I.
Step 4: To a solution of tert-butyl 4-[4-[[5-[2-[tert-butyl(diphenyl)silyl]oxyethoxy]pyrimidin-2-yl]-(trideuteriomethyl)amino]-2-chloro-phenyl]piperazine-1-carboxylate (126 mg, 179 μmol) in dichloromethane (2 mL) was added trifluoroacetic acid (1.0 mL, 13 mmol) and the mixture was stirred at room temperature for 1 hour. The mixture was concentrated to dryness and dissolved in EtOAc. The mixture was neutralized with 1N aq. NaOH solution and extracted with EtOAc. The combined organic layers were washed with brine, dried over MgSO₄, filtered, and concentrated. The residue was purified by FCC on SiO₂ using a gradient of 1-20% CH₃OH in CH₂Cl₂. 37-4 was collected as a brown solid in 80% yield (86 mg, 0.14 mmol). LC/MS found 605.5 [M+H]⁺ and 303.3 [M+2H]²⁺.
Step 5: A mixture of 5-[2-[tert-butyl(diphenyl)silyl]oxyethoxy]-N-(3-chloro-4-piperazin-1-yl-phenyl)-N-(trideuteriomethyl)pyrimidin-2-amine (86 mg, 140 μmol), 2-chloro-N-[2-[(3S)-2,6-dioxo-3-piperidyl]-1-oxo-isoindolin-5-yl]acetamide (53 mg, 160 μmol), and potassium carbonate (40 mg, 290 μmol) in DMF (2.0 mL) was heated to 70° C. with stirring for 4 hours then cooled to room temperature, poured over H₂O, and extracted with EtOAc. The combined organic layers were washed with brine, dried over Na₂SO₄, filtered, and concentrated. The residue was purified by FCC on SiO₂ using a gradient of 1-10% CH₃OH in CH₂Cl₂, then by C-18 reverse-phase chromatography using a gradient of 5-95% CH₃CN in H₂O with 0.1% HCO₂H. 37 was collected as a white solid in 11% yield (10 mg, 15 μmol). TLC (10% CH₃OH/CH₂Cl₂): R_{f}=0.43. LC/MS found 666.4 [M+H]⁺ and 333.6 [M+2H]²⁺.

### Method 90

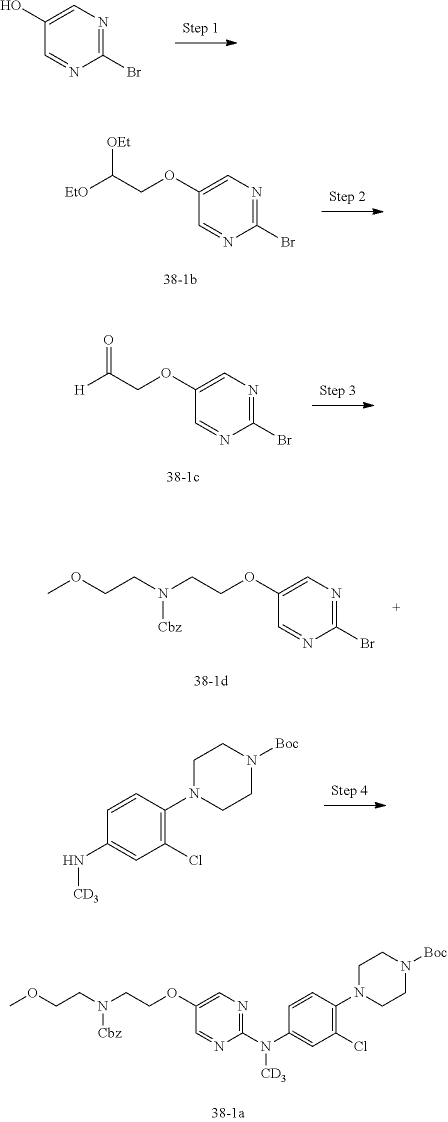 [SMILES: Oc1cnc(Br)nc1 ; CCOC(COc1cnc(Br)nc1)OCC ; O=CCOc1cnc(Br)nc1 ; COCCN(CCOc1cnc(Br)nc1)C(=O)OCc1ccccc1 ; [2H]C([2H])([2H])Nc1ccc(N2CCN(C(=O)OC(C)(C)C)CC2)c(Cl)c1 ; [2H]C([2H])([2H])N(c1ccc(N2CCN(C(=O)OC(C)(C)C)CC2)c(Cl)c1)c1ncc(OCCN(CCOC)C(=O)OCc2ccccc2)cn1]

Step 1: To a mixture of 2,2-diethoxyethanol (0.78 mL, 5.7 mmol), 2-bromopyrimidin-5-ol (502 mg, 2.87 mmol), and triphenylphosphine (835 mg, 3.18 mmol) in toluene (8.0 mL) was added bis(2-methoxyethyl) azodicarboxylate (735 mg, 3.14 mmol). The mixture was degassed via sparging with N₂ for 5 minutes and heated to 100° C. while stirring overnight. The mixture was poured over H₂O and extracted with EtOAc. The combined organic layers were washed with 1N aq. NaOH solution (2 times), washed with H₂O, washed with brine, dried over Na₂SO₄, filtered, and concentrated. The residue was purified by FCC on SiO₂ using a gradient of 10-30% EtOAc in heptanes. 38-1b was collected as a white solid in 73% yield (607 mg, 2.08 mmol). TLC (30% EtOAc/heptanes): R_{f}=0.39. LC/MS found 291.1 [M+H]⁺.
Step 2: To a solution of 2-bromo-5-(2,2-diethoxyethoxy)pyrimidine (322 mg, 1.11 mmol) in THF (5 mL) was added aq. hydrochloric acid (1 N, 5.00 mmol, 5 mL). The mixture was heated to 70° C. with stirring for 2 hours. The mixture was cooled to room temperature, neutralized with a sat. aq. NaHCO₃ solution, and extracted with EtOAc. The combined organic layers were washed with brine, dried over Na₂SO₄, filtered, and concentrated. Crude 38-1c was used without further purification. LC/MS found 217.0 [M+H]⁺ and 235.1 [M+H+H₂O]⁺.
Step 3: To a solution of 2-(2-bromopyrimidin-5-yl)oxyacetaldehyde (240 mg, 1.11 mmol) and 2-methoxyethanamine (0.20 mL, 2.3 mmol) in dichloromethane (10 mL) was added sodium triacetoxyborohydride (498 mg, 2.35 mmol) and the mixture was stirred at room temperature overnight. The mixture was then cooled to 0° C. and triethylamine (0.92 mL, 6.6 mmol) followed by benzyl chloroformate (0.35 mL, 2.5 mmol) were added. Stirring continued for 1 hour before quenching with a sat. aq. NaHCO₃ solution. The layers were separated and the aqueous was extracted with CH₂Cl₂. The combined organic layers were washed with brine, dried over Na₂SO₄, filtered, and concentrated. The residue was purified by FCC on SiO₂ using a gradient of 20-60% EtOAc in hexanes. 38-1d was collected as a colorless oil in 38% yield (174 mg, 0.42 mmol). TLC (50% EtOAc/heptanes): R_{f}=0.25. LC/MS found 410.1 [M+H]⁺.
Step 4: A mixture of benzyl N-[2-(2-bromopyrimidin-5-yl)oxyethyl]-N-(2-methoxyethyl)carbamate (174 mg, 0.42 mmol), tert-butyl 4-[2-chloro-4-(trideuteriomethylamino)phenyl]piperazine-1-carboxylate (150 mg, 0.46 mmol), methanesulfonato(2-dicyclohexylphosphino-2′,6′-di-i-propoxy-1,1′-biphenyl)(2′-methylamino-1,1′-biphenyl-2-yl)palladium(II) (18 mg, 21 μmol), 2-dicyclohexylphosphino-2′,6′-di-i-propoxy-1,1′-biphenyl (10 mg, 21 μmol), and cesium carbonate (294 mg, 0.90 mmol) in dry 1,4-dioxane (4.0 mL) was degassed via sparging with N₂ for 10 minutes then heated to 80° C. with stirring overnight. The mixture was cooled to room temperature and filtered through a pad of diatomaceous earth, and the filtrate was concentrated to dryness. The residue was purified by flash column chromatography using a gradient of 30-70% EtOAc in hexanes. 38-1a was collected as a brown semi-solid with 85% purity in 52% yield (171 mg, 0.22 mmol). TLC (50% EtOAc/hexanes): R_{f}=0.19, TLC (EtOAc): R_{f}=0.62. LC/MS D.N.I.

### Method 91

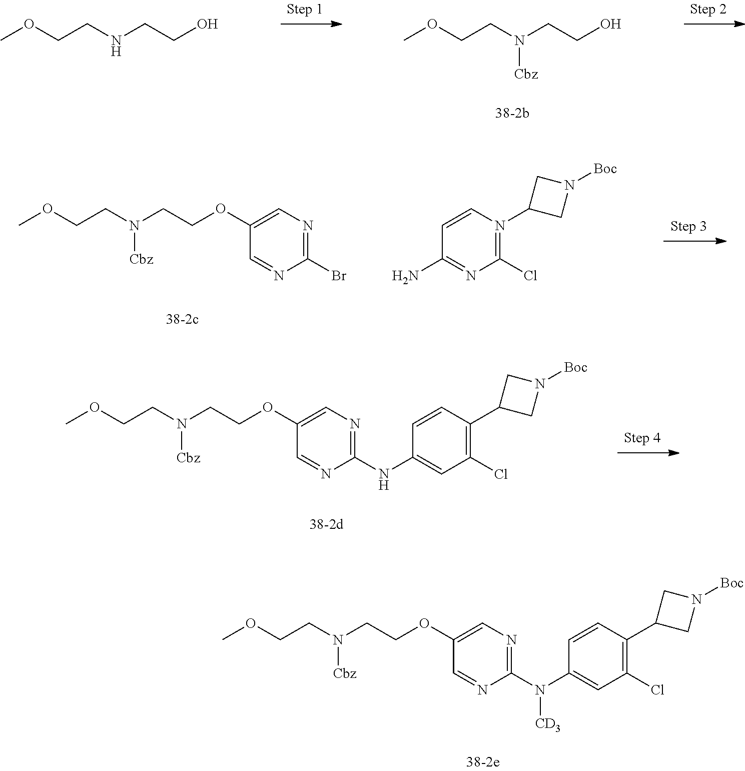

Step 1: To a solution of 2-(2-methoxyethylamino)ethanol (0.86 g, 7.22 mmol) and diisopropylethylamine (3.8 mL, 22 mmol) in dichloromethane (18 mL) cooled to 0° C. was added dropwise benzyl carbonochloridate (1.05 mL, 7.45 mmol). The mixture was stirred for 1 hour then quenched with a sat. aq. NaHCO₃ solution. The layers were separated and the aqueous was washed with H₂O, washed with brine, dried over Na₂SO₄, filtered, and concentrated. The residue was purified by FCC on SiO₂ using a gradient of 50-100% EtOAc in hexanes. 38-2b was collected as a colorless oil in 82% yield (1.50 g, 5.92 mmol). TLC (50% EtOAc/heptanes): R_{f}=0.12, TLC (EtOAc): R_{f}=0.37. LC/MS found 254.3 [M+H]⁺.
Step 2: To a mixture of benzyl N-(2-hydroxyethyl)-N-(2-methoxyethyl)carbamate (1.50 g, 5.92 mmol), 2-bromopyrimidin-5-ol (1.24 g, 7.11 mmol), and triphenylphosphine (1.86 g, 7.11 mmol) in toluene (12 mL) was added bis(2-methoxyethyl) azodicarboxylate (1.67 g, 7.13 mmol). The mixture was degassed via sparging with N₂ for 5 minutes and heated to 100° C. while stirring overnight. The mixture was poured over H₂O and extracted with EtOAc. The combined organic layers were washed with 1N aq. NaOH solution (2 times), washed with H₂O, washed with brine, dried over Na₂SO₄, filtered, and concentrated. The residue was purified by FCC on SiO₂ using a gradient of 30-70% EtOAc in heptanes. 38-2c was collected as a colorless oil with 75% purity in 41% yield (1.34 g, 2.45 mmol). TLC (50% EtOAc/heptanes): R_{f}=0.24. LC/MS found 410.2 [M+H]⁺.
Step 3: A mixture of benzyl N-[2-(2-bromopyrimidin-5-yl)oxyethyl]-N-(2-methoxyethyl)carbamate (710 mg, 1.73 mmol), tert-butyl 3-(4-amino-2-chloro-phenyl)azetidine-1-carboxylate (510 mg, 1.80 mmol), methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II) (82 mg, 89 μmol), 2-(dicyclohexylphosphino)-3,6-dimethoxy-2′,4′,6′-tri-i-propyl-1,1′-biphenyl (48 mg, 89 μmol), and cesium carbonate (1.13 g, 3.46 mmol) in dry 1,4-dioxane (16 mL) was degassed via sparging with N₂ for 10 minutes then heated to 100° C. with stirring overnight. The mixture was cooled to room temperature and filtered through a pad of diatomaceous earth, and the filtrate was concentrated to dryness. The residue was purified by flash column chromatography using a gradient of 20-60% EtOAc in CH₂Cl₂, then by reverse phase C-18 chromatography using a gradient of 60-95% CH₃CN in H₂O with 0.1% HCO₂H. 38-2d was collected as an off-white semi-solid in 45% yield (473 mg, 0.77 mmol). TLC (70% EtOAc/hexanes): R_{f}=0.32, TLC (40% EtOAc/CH₂Cl₂): R_{f}=0.30. LC/MS D.N.I. ¹H NMR (400 MHz, DMSO-d₆) δ ppm 9.64 (s, 1H), 8.30 (d, J=27.4 Hz, 2H), 7.97 (d, J=2.2 Hz, 1H), 7.60 (br d, J=8.1 Hz, 1H), 7.39 (d, J=8.6 Hz, 1H), 7.33 (m, 5H), 5.07 (s, 2H), 4.18 (m, 4H), 3.99 (m, 1H), 3.89 (t, J=7.6 Hz, 2H), 3.62 (m, 2H), 3.47 (br s, 4H), 3.22 (d, J=10.3 Hz, 3H), 1.38 (s, 9H).
Step 4: To a mixture of tert-butyl 3-[4-[[5-[2-[benzyloxycarbonyl(2-methoxyethyl)amino]ethoxy]pyrimidin-2-yl]amino]-2-chloro-phenyl]azetidine-1-carboxylate (242 mg, 0.40 mmol) in dry DMF (2.0 mL) cooled to 0° C. was added sodium hydride (in oil dispersion) 60% dispersion in mineral oil (39 mg, 1.6 mmol). The mixture was stirred for 30 minutes and iodomethane-d3 (0.0.8 mL, 1.3 mmol) was added. The mixture was warmed to 40° C. and stirred overnight, then poured over H₂O, and extracted with EtOAc. The combined organic layers were washed with brine, dried over Na₂SO₄, filtered, and concentrated. The residue was purified by FCC on SiO₂ using a gradient of 30-70% EtOAc in hexanes. 38-2a was collected as a colorless semi-solid in 66% yield (177 mg, 0.26 mmol). TLC (50% EtOAc/heptanes): R_{f}=0.20, TLC (70% EtOAc/heptanes): R_{f}=0.39. LC/MS D.N.I. [M+H]⁺.

### General Scheme 29

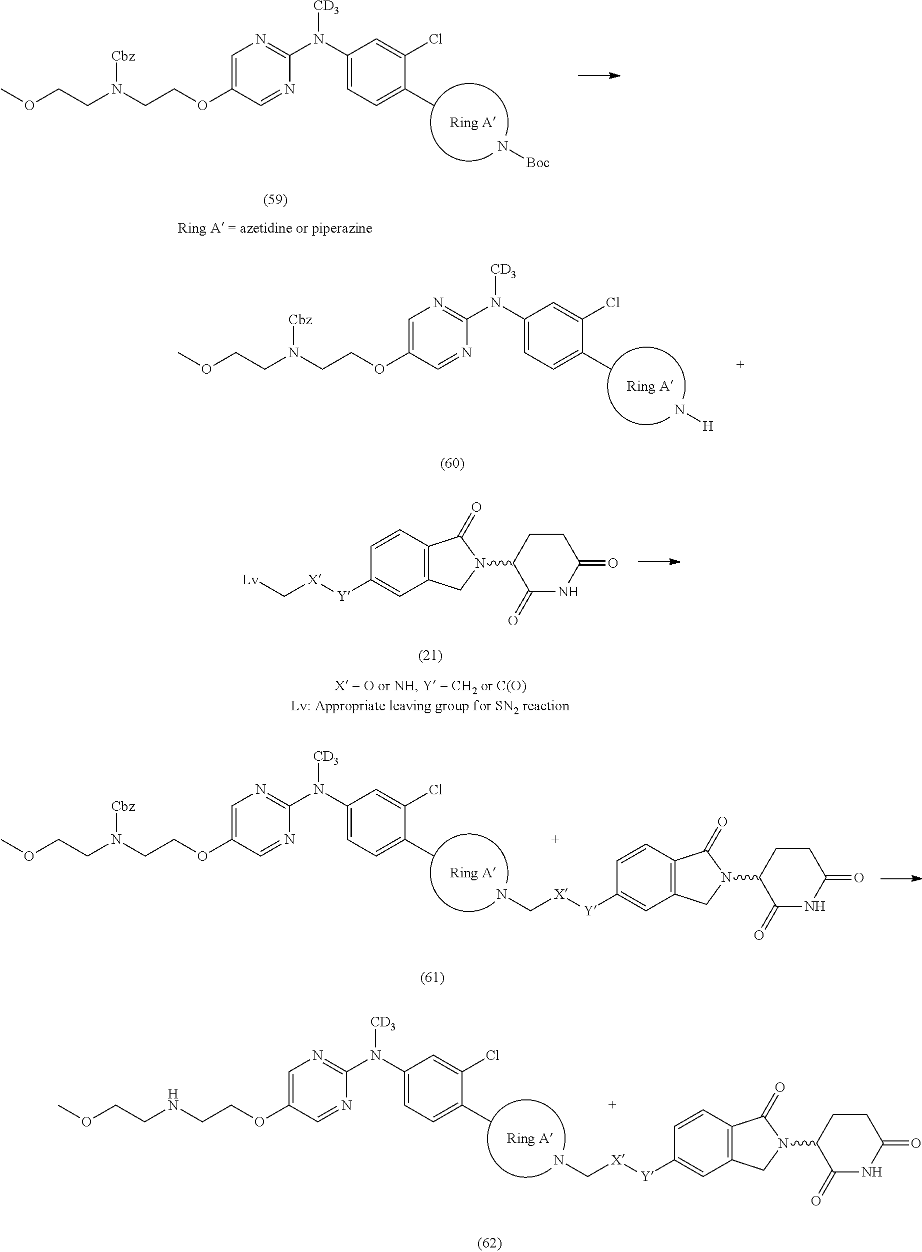

### Method 92

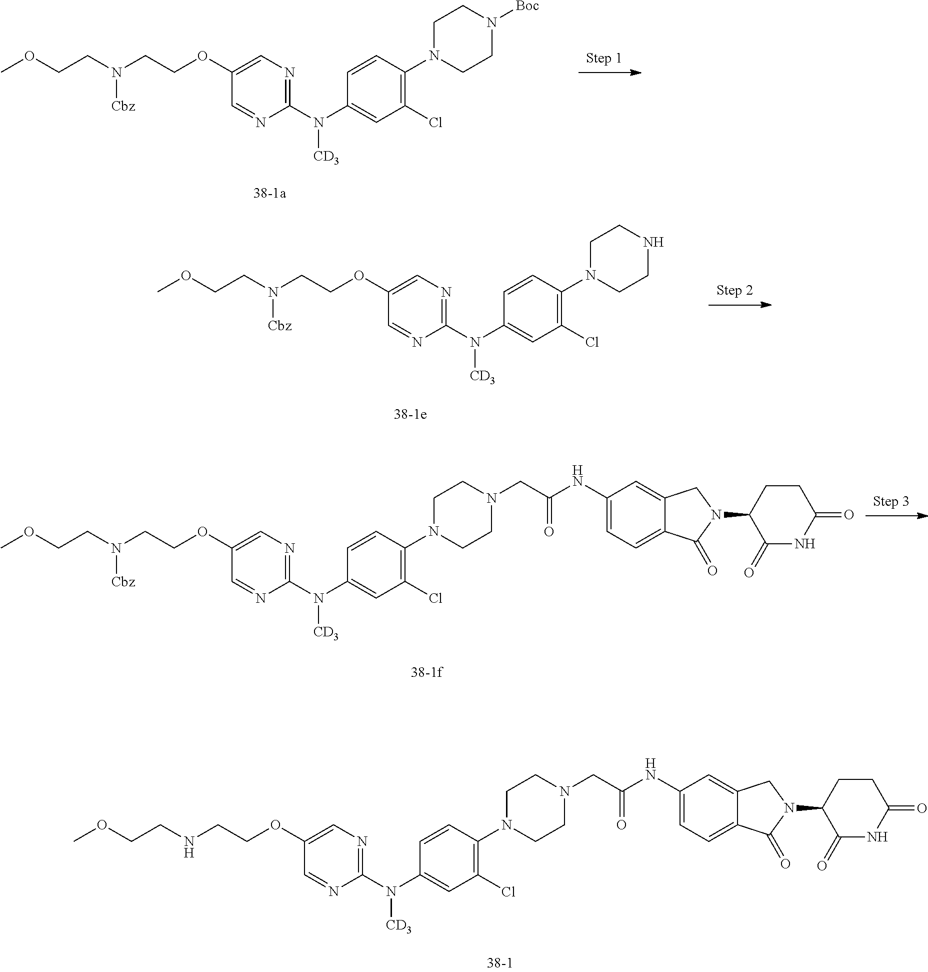 [SMILES: [2H]C([2H])([2H])N(c1ccc(N2CCN(C(=O)OC(C)(C)C)CC2)c(Cl)c1)c1ncc(OCCN(CCOC)C(=O)OCc2ccccc2)cn1 ; [2H]C([2H])([2H])N(c1ccc(N2CCNCC2)c(Cl)c1)c1ncc(OCCN(CCOC)C(=O)OCc2ccccc2)cn1 ; [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCN(CCOC)C(=O)OCc2ccccc2)cn1 ; [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCNCCOC)cn1]

Step 1: To a solution of tert-butyl 4-[4-[[5-[2-[benzyloxycarbonyl(2-methoxyethyl)amino]ethoxy]pyrimidin-2-yl]-(trideuteriomethyl)amino]-2-chloro-phenyl]piperazine-1-carboxylate (171 mg, 220 μmol) in dichloromethane (2 mL) was added trifluoroacetic acid (1.0 mL, 13 mmol) and the mixture was stirred at room temperature for 1 hour. The mixture was concentrated to dryness, neutralized with a 1000 aq. K₂CO₃ solution, and extracted with EtOAc. The combined organic layers were washed with brine, dried over MgSO₄, filtered, and concentrated. The residue was purified by FCC on SiO₂ using a gradient of 5-50% CH₃OH in CH₂Cl₂. 38-1e was collected as a white solid in 43% yield (53 mg, 95 μmol). TLC (20% CH₃OH/CH₂Cl₂): R_{f}=0.17. LC/MS found 558.6 [M+H]⁺, 468.3 [M+H−Bn]⁺, and 279.7 [M+2H]²⁺.
Step 2: A mixture of benzyl N-[2-[2-[3-chloro-4-piperazin-1-yl-N-(trideuteriomethyl)anilino]pyrimidin-5-yl]oxyethyl]-N-(2-methoxyethyl)carbamate (53 mg, 95 μmol), 2-chloro-N-[2-[(3S)-2,6-dioxo-3-piperidyl]-1-oxo-isoindolin-5-yl]acetamide (36 mg, 110 μmol), and potassium carbonate (30 mg, 220 μmol) in DMF (2.0 mL) was heated to 70° C. with stirring for 2 hours then cooled to room temperature, poured over H₂O, and extracted with EtOAc. The combined organic layers were washed with brine, dried over Na₂SO₄, filtered, and concentrated. The residue was purified by FCC on SiO₂ using a gradient of 1-10% CH₃OH in CH₂Cl₂. 38-1f was collected as a white solid with 92% purity in 35% yield (31 mg, 33 μmol). TLC (5% CH₃OH/CH₂Cl₂): R_{f}=0.17, TLC (10% CH₃OH/CH₂Cl₂): R_{f}=0.54. LC/MS found 857.5 [M+H]⁺, 767.4 [M+H−Bn]⁺, and 429.4 [M+2H]²⁺.
Step 3: A solution of benzyl N-[2-[2-[3-chloro-4-[4-[2-[[2-[(3S)-2,6-dioxo-3-piperidyl]-1-oxo-isoindolin-5-yl]amino]-2-oxo-ethyl]piperazin-1-yl]-N-(trideuteriomethyl)anilino]pyrimidin-5-yl]oxyethyl]-N-(2-methoxyethyl)carbamate (31 mg, 33 μmol) in trifluoroacetic acid (2.0 mL, 26 mmol) was stirred at 60° C. for 2 hours. The mixture was concentrated to dryness, neutralized with a 10% aq. K₂CO₃ solution, and extracted with EtOAc. The combined organic layers were washed with brine, dried over MgSO₄, filtered, and concentrated. The residue was purified by FCC on SiO₂ using a gradient of 1-15% CH₃OH (with 3% Et₃N) in CH₂Cl₂. Compound 38-1 was collected as a white solid with 93% purity in 46% yield (12 mg, 15 mol). TLC (10% CH₃OH (w/3% Et₃N)/CH₂Cl₂): R_{f}=0.25. LC/MS found 723.4 [M+H]⁺ and 362.4 [M+2H]²⁺.

The following compounds 38-1 through 38-2 (62) shown in Table 22 were prepared by methods similar to those described for the preparation of 38-1 (its intermediate 38-1a), using the appropriate intermediates (59 and 21).

TABLE 22

| Compd | Starting material | |
|---|---|---|
| | 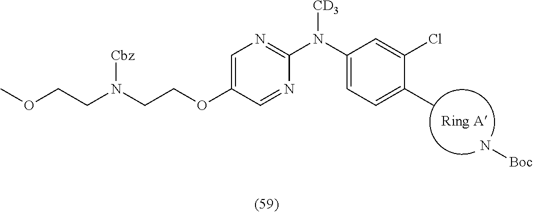 [SMILES: [2H]C([2H])([2H])N(c1ccc(C)c(Cl)c1)c1ncc(OCCN(CCOC)C(=O)OCc2ccccc2)cn1 ; CC(C)(C)OC(N)=O] | 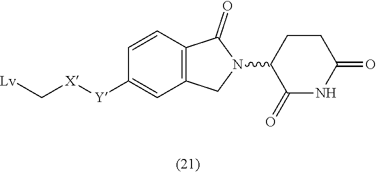 |
| 38-1 | 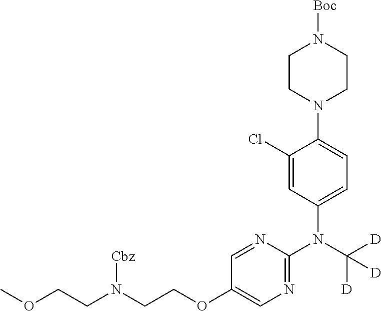 [SMILES: [2H]C([2H])([2H])N(c1ccc(N2CCN(C(=O)OC(C)(C)C)CC2)c(Cl)c1)c1ncc(OCCN(CCOC)C(=O)OCc2ccccc2)cn1] | 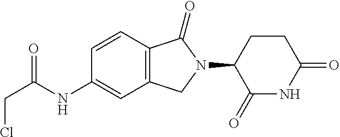 [SMILES: O=C1CC[C@H](N2Cc3cc(NC(=O)CCl)ccc3C2=O)C(=O)N1] |
| 38-2 | 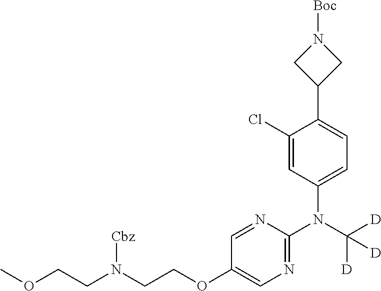 [SMILES: [2H]C([2H])([2H])N(c1ccc(C2CN(C(=O)OC(C)(C)C)C2)c(Cl)c1)c1ncc(OCCN(CCOC)C(=O)OCc2ccccc2)cn1] | 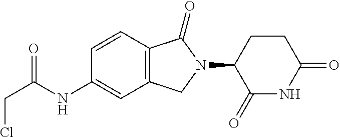 [SMILES: O=C1CC[C@H](N2Cc3cc(NC(=O)CCl)ccc3C2=O)C(=O)N1] |
| | | MS |
| | | found |
| Compd | Product | [M + H]+ |
| | 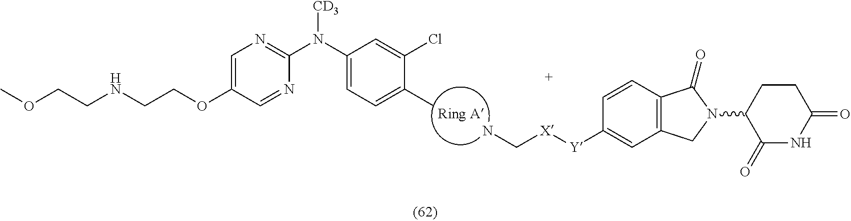 | |
| 38-1 | 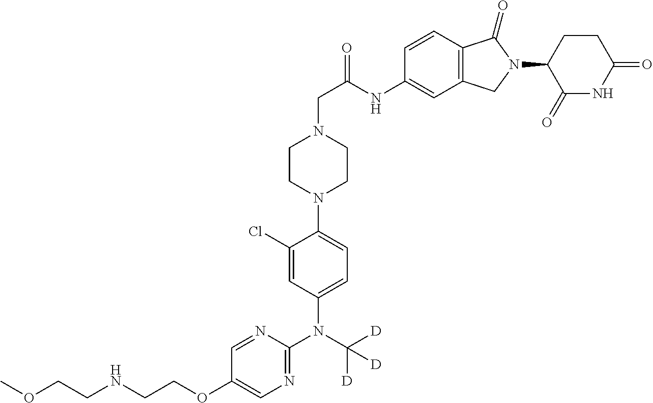 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCNCCOC)cn1] | 723.4 |
| 38-2 | 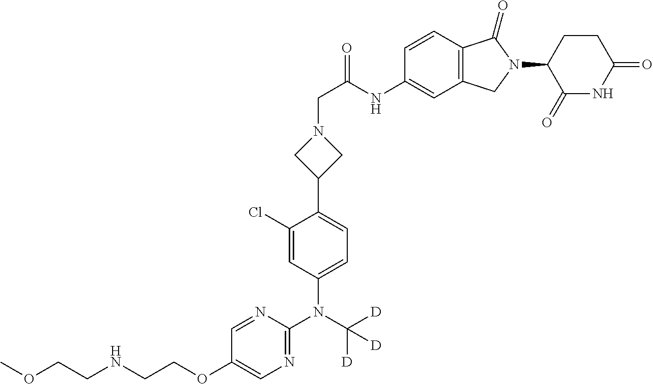 [SMILES: [2H]C([2H])([2H])N(c1ccc(C2CN(CC(=O)Nc3ccc4c(c3)CN([C@H]3CCC(=O)NC3=O)C4=O)C2)c(Cl)c1)c1ncc(OCCNCCOC)cn1] | 694.4 |

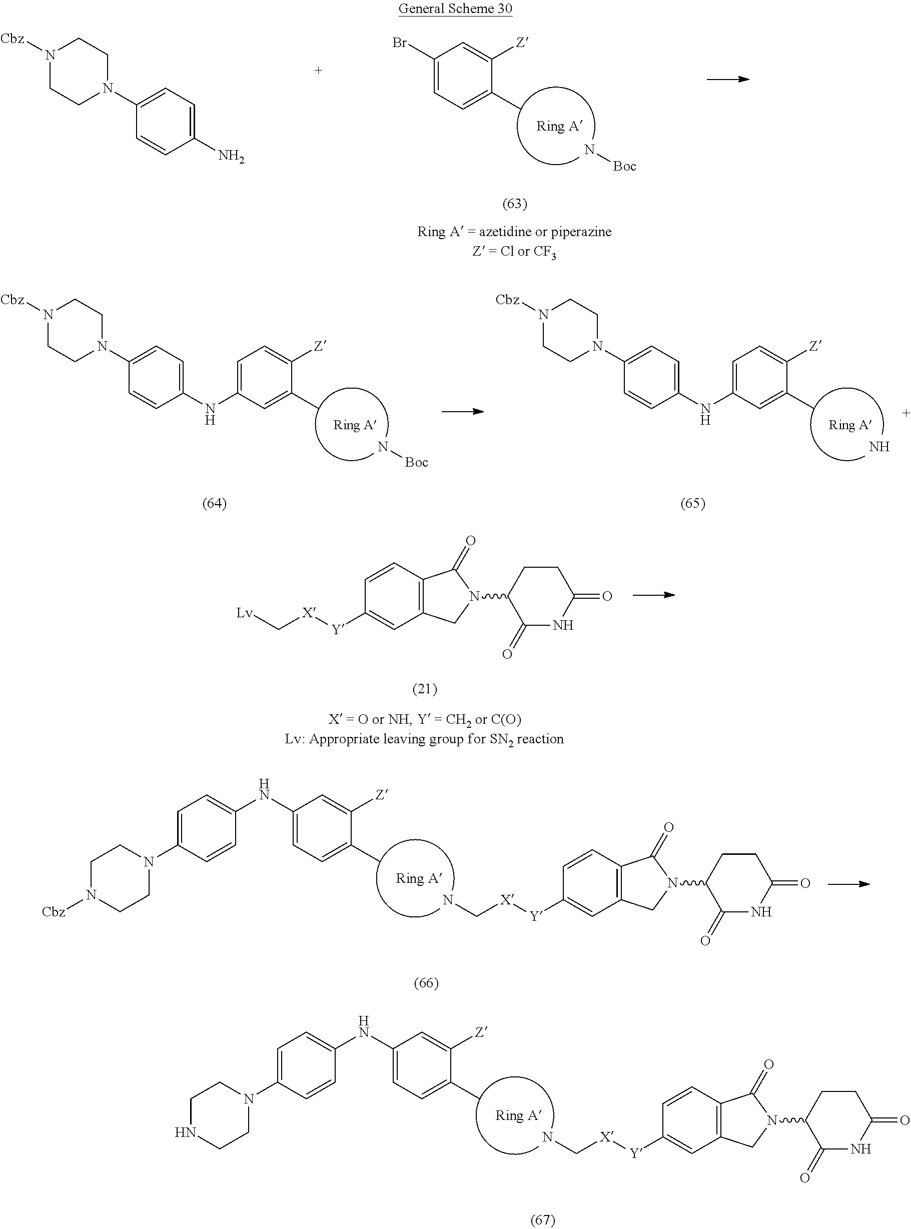

### Method 93

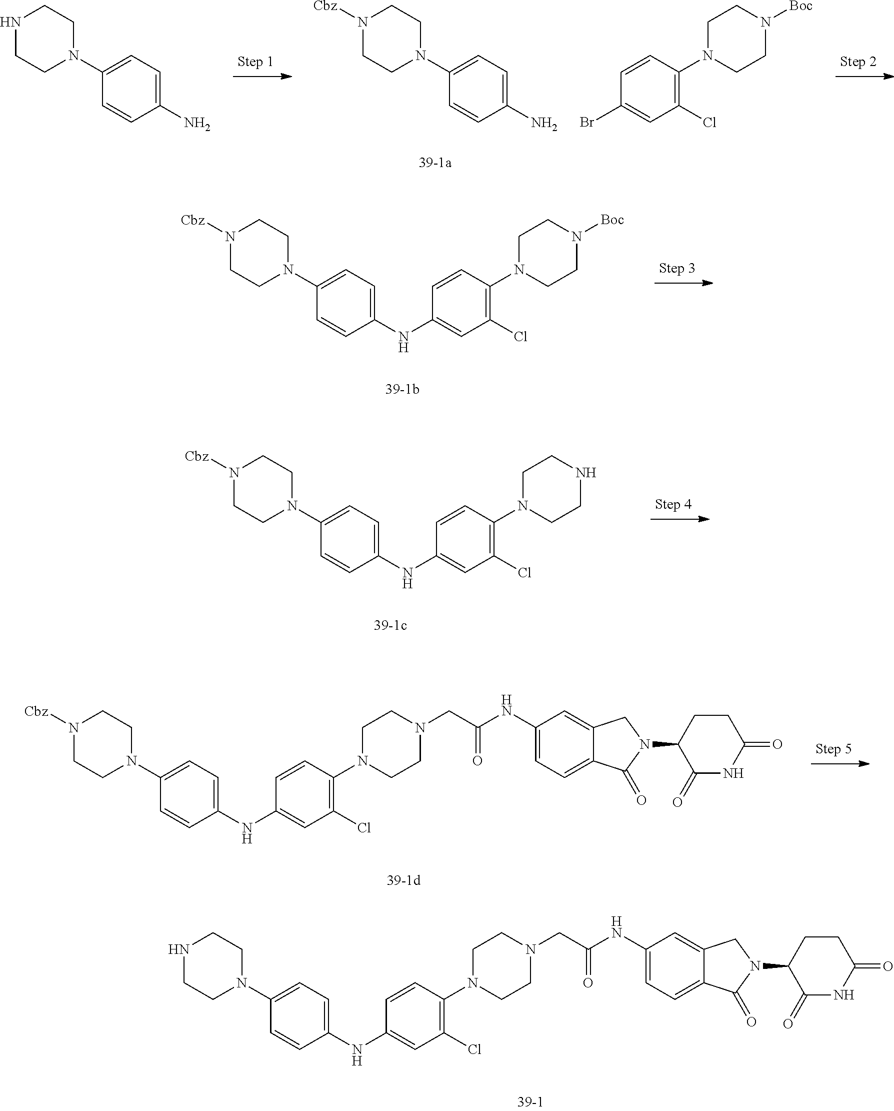

Step 1: To a solution of 4-piperazin-1-ylaniline (4.57 g, 25.8 mmol) and triethylamine (9.0 mL, 65 mmol) in dichloromethane (30 mL) cooled to 0° C. was added dropwise benzyl carbonochloridate (3.0 mL, 21 mmol). The mixture was stirred for 1 hour then quenched with a sat. aq. NaHCO₃ solution. The layers were separated and the aqueous was washed with H₂O, washed with brine, dried over Na₂SO₄, filtered, and concentrated. The residue was purified by FCC on SiO₂ using a gradient of 50-100% EtOAc in hexanes. 39-1a was collected as a brown solid in 13% yield (890 mg, 2.86 mmol). TLC (EtOAc): R_{f}=0.46. LC/MS found 312.1 [M+H]⁺.
Step 2: A mixture of benzyl 4-(4-aminophenyl)piperazine-1-carboxylate (513 mg, 1.10 mmol), tert-butyl 4-(4-bromo-2-chloro-phenyl)piperazine-1-carboxylate (415 mg, 1.10 mmol), methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II) (25 mg, 27 μmol), 2-(dicyclohexylphosphino)-3,6-dimethoxy-2′,4′,6′-tri-i-propyl-1,1′-biphenyl (15 mg, 28 μmol), and cesium carbonate (720 mg, 2.21 mmol) in dry 1,4-dioxane (6 mL) was degassed via sparging with N₂ for 10 minutes then heated to 80° C. with stirring for 4 hours. The mixture was cooled to room temperature and filtered through a pad of diatomaceous earth, and the filtrate was concentrated to dryness. The residue was purified by flash column chromatography using a gradient of 1-20% EtOAc in CH₂Cl₂. 39-1b was collected as a pale pink solid in 67% yield (451 mg, 0.74 mmol). TLC (10% EtOAc/CH₂Cl₂): R_{f}=0.17, TLC (20% EtOAc/CH₂Cl₂): R_{f}=0.40. LC/MS found 606.5 [M+H]⁺.
Step 3: To a solution of benzyl 4-[4-[4-(4-tert-butoxycarbonylpiperazin-1-yl)-3-chloro-anilino]phenyl]piperazine-1-carboxylate (451 mg, 0.74 mmol) in dichloromethane (4 mL) was added trifluoroacetic acid (2.0 mL, 26 mmol) and the mixture was stirred at room temperature for 1 hour. The mixture was poured over 10% aq. K₂CO₃ solution with brine, extracted with 10% i-PrOH in CH₂Cl₂. The combined organic layers were washed with brine, dried over MgSO₄, filtered, and concentrated 39-1c was collected as a foamy pale yellow solid and used without further purification. LC/MS found 506.4 [M+H]⁺.
Step 4: A mixture of benzyl 4-[4-(3-chloro-4-piperazin-1-yl-anilino)phenyl]piperazine-1-carboxylate (188 mg, 0.38 mmol), 2-chloro-N-[2-[(3S)-2,6-dioxo-3-piperidyl]-1-oxo-isoindolin-5-yl]acetamide (132 mg, 0.39 mmol), and potassium carbonate (105 mg, 0.76 mmol) in DMF (2 mL) was heated to 70° C. with stirring for 2 hours then cooled to room temperature, poured over H₂O, and extracted with 10% i-PrOH in EtOAc. The combined organic layers were washed with H₂O (3 times), washed with brine, dried over MgSO₄, filtered, and concentrated. The residue was purified by FCC on SiO₂ using a gradient of 1-10% CH₃OH in CH₂Cl₂. 39-1d was collected as a white solid in 43% yield (128 mg, 0.16 mmol). TLC (7% CH₃OH/CH₂Cl₂): R_{f}=0.40. LC/MS found 805.5 [M+H]⁺ and 403.4 [M+2H]²⁺.
Step 5: A solution of benzyl 4-[4-[3-chloro-4-[4-[2-[[2-[(3S)-2,6-dioxo-3-piperidyl]-1-oxo-isoindolin-5-yl]amino]-2-oxo-ethyl]piperazin-1-yl]anilino]phenyl]piperazine-1-carboxylate (128 mg, 0.16 mmol) in trifluoroacetic acid (4.0 mL, 52 mmol) was stirred at 60° C. for 2 hours. The mixture was cooled to room temperature, poured over 10% aq. K₂CO₃/brine, and extracted with 20% i-PrOH in CH₂Cl₂. The combined organic layers were washed with brine, dried over MgSO₄, filtered, and concentrated. The residue was purified by reverse phase C-18 chromatography using a gradient of 5-95% CH₃CN in H₂O with 0.1% HCO₂H. 39-1 was collected as a white solid in 39% yield (44 mg, 61 μmol). LC/MS found 671.4 [M+H]⁺ and 336.3 [M+2H]²⁺.

The following compounds 39-1 through 39-5 (67) shown in Table 23 were prepared by methods similar to those described for the preparation of 39-1 (its intermediate 39-1a), using the appropriate intermediates (64 and 21).

TABLE 23

| Compd | Starting material | |
|---|---|---|
| | 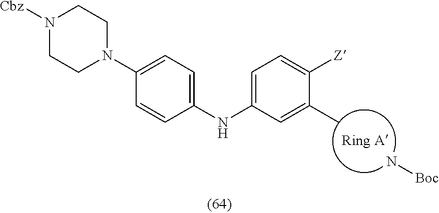 | 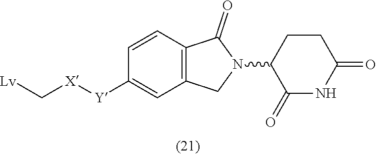 |
| 39-1 | 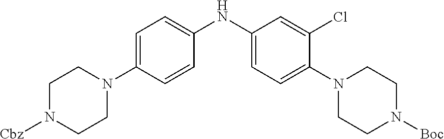 [SMILES: CC(C)(C)OC(=O)N1CCN(c2ccc(Nc3ccc(N4CCN(C(=O)OCc5ccccc5)CC4)cc3)cc2Cl)CC1] | 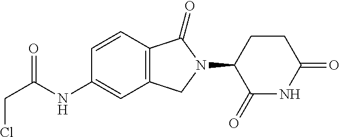 [SMILES: O=C1CC[C@H](N2Cc3cc(NC(=O)CCl)ccc3C2=O)C(=O)N1] |
| 39-2 | 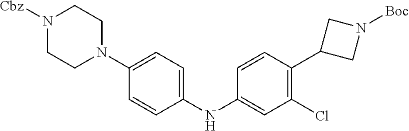 [SMILES: CC(C)(C)OC(=O)N1CC(c2ccc(Nc3ccc(N4CCN(C(=O)OCc5ccccc5)CC4)cc3)cc2Cl)C1] | 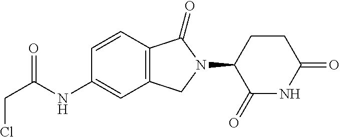 [SMILES: O=C1CC[C@H](N2Cc3cc(NC(=O)CCl)ccc3C2=O)C(=O)N1] |
| 39-3 | 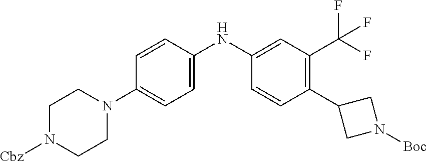 [SMILES: CC(C)(C)OC(=O)N1CC(c2ccc(Nc3ccc(N4CCN(C(=O)OCc5ccccc5)CC4)cc3)cc2C(F)(F)F)C1] | 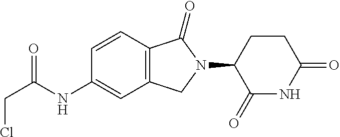 [SMILES: O=C1CC[C@H](N2Cc3cc(NC(=O)CCl)ccc3C2=O)C(=O)N1] |
| 39-4 | 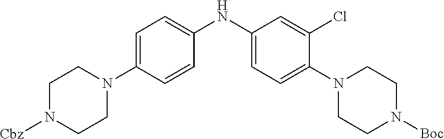 [SMILES: CC(C)(C)OC(=O)N1CCN(c2ccc(Nc3ccc(N4CCN(C(=O)OCc5ccccc5)CC4)cc3)cc2Cl)CC1] | 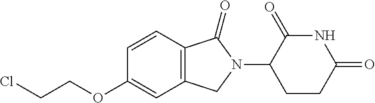 [SMILES: O=C1CCC(N2Cc3cc(OCCCl)ccc3C2=O)C(=O)N1] |
| 39-5 | 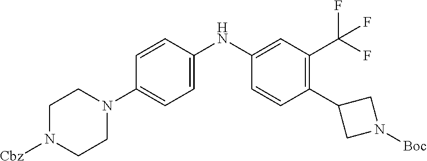 [SMILES: CC(C)(C)OC(=O)N1CC(c2ccc(Nc3ccc(N4CCN(C(=O)OCc5ccccc5)CC4)cc3)cc2C(F)(F)F)C1] | 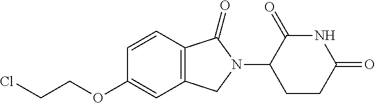 [SMILES: O=C1CCC(N2Cc3cc(OCCCl)ccc3C2=O)C(=O)N1] |
| | | MS |
| | | found |
| Compd | Product | [M + H]+ |
| | 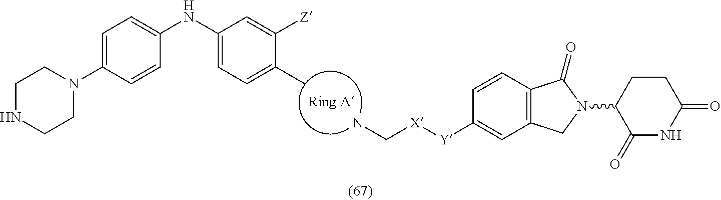 | |
| 39-1 | 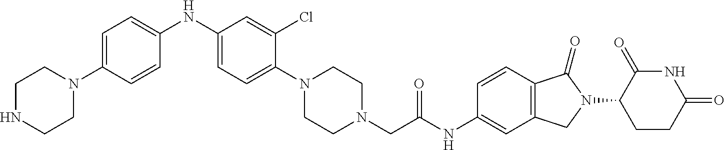 [SMILES: O=C1CC[C@H](N2Cc3cc(NC(=O)CN4CCN(c5ccc(Nc6ccc(N7CCNCC7)cc6)cc5Cl)CC4)ccc3C2=O)C(=O)N1] | 671.4 |
| 39-2 | 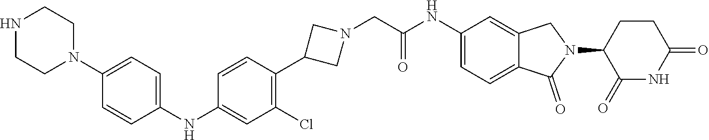 [SMILES: O=C1CC[C@H](N2Cc3cc(NC(=O)CN4CC(c5ccc(Nc6ccc(N7CCNCC7)cc6)cc5Cl)C4)ccc3C2=O)C(=O)N1] | 642.4 |
| 39-3 | 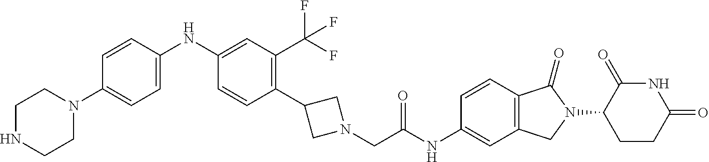 [SMILES: O=C1CC[C@H](N2Cc3cc(NC(=O)CN4CC(c5ccc(Nc6ccc(N7CCNCC7)cc6)cc5C(F)(F)F)C4)ccc3C2=O)C(=O)N1] | 676.4 |
| 39-4 | 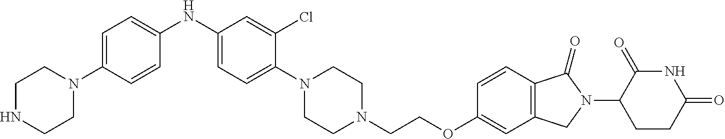 [SMILES: O=C1CCC(N2Cc3cc(OCCN4CCN(c5ccc(Nc6ccc(N7CCNCC7)cc6)cc5Cl)CC4)ccc3C2=O)C(=O)N1] | 658.4 |
| 39-5 | 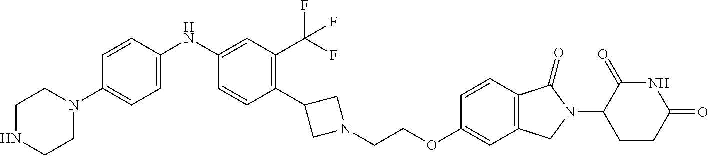 [SMILES: O=C1CCC(N2Cc3cc(OCCN4CC(c5ccc(Nc6ccc(N7CCNCC7)cc6)cc5C(F)(F)F)C4)ccc3C2=O)C(=O)N1] | 663.5 |

### Table 23

### Method 94

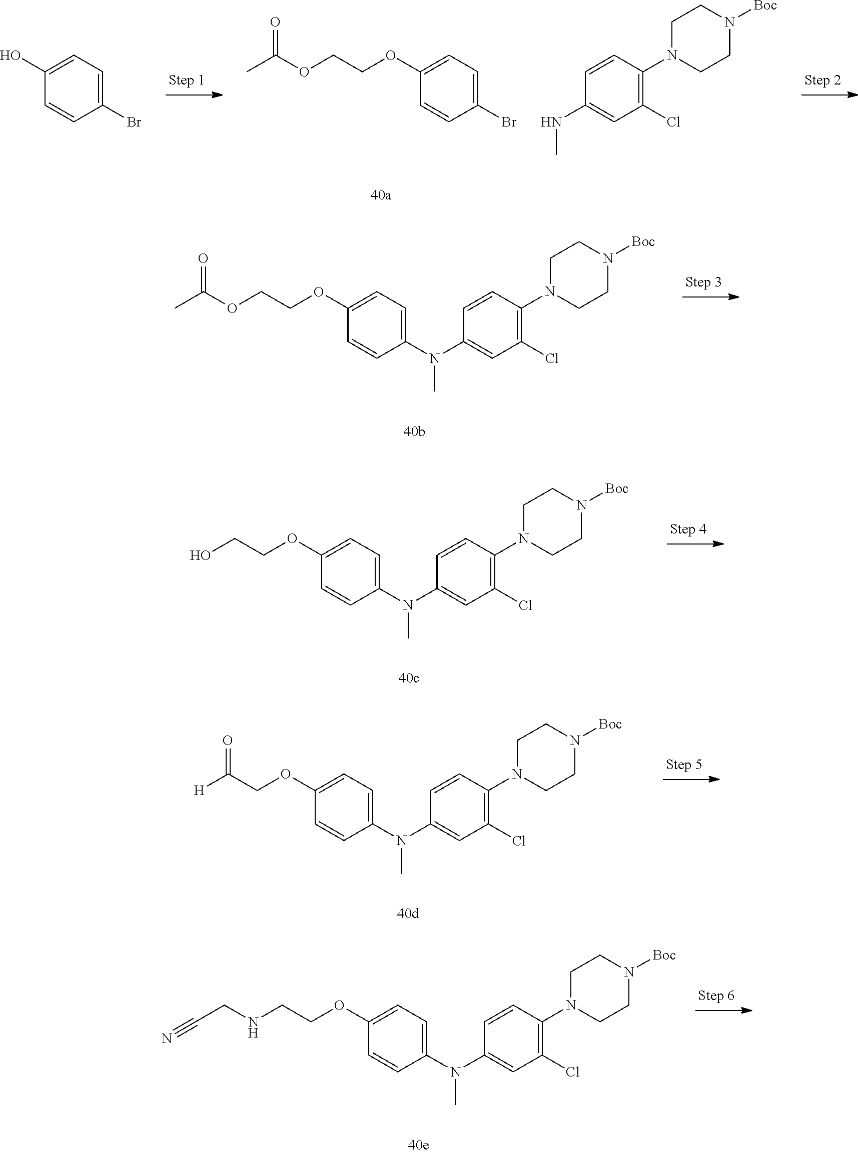

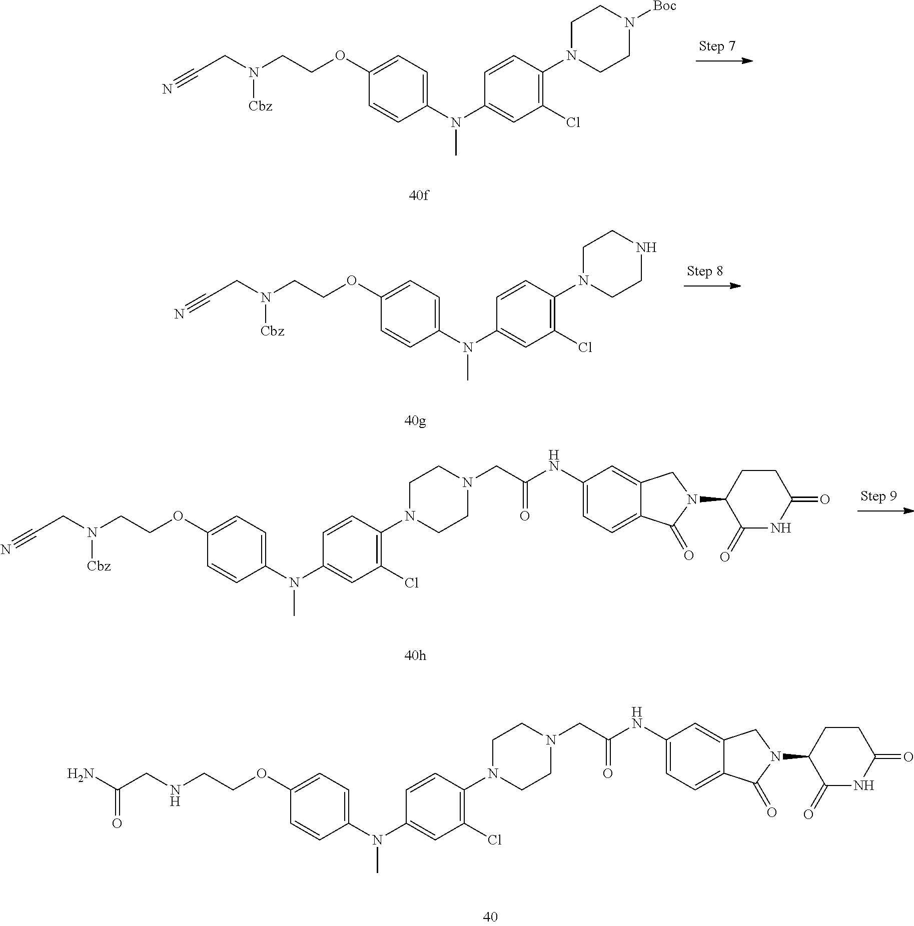 [SMILES: CN(c1ccc(OCCN(CC#N)C(=O)OCc2ccccc2)cc1)c1ccc(N2CCN(C(=O)OC(C)(C)C)CC2)c(Cl)c1 ; CN(c1ccc(OCCN(CC#N)C(=O)OCc2ccccc2)cc1)c1ccc(N2CCNCC2)c(Cl)c1 ; CN(c1ccc(OCCN(CC#N)C(=O)OCc2ccccc2)cc1)c1ccc(N2CCN(CC(=O)Nc3ccc4c(c3)CN([C@H]3CCC(=O)NC3=O)C4=O)CC2)c(Cl)c1 ; CN(c1ccc(OCCNCC(N)=O)cc1)c1ccc(N2CCN(CC(=O)Nc3ccc4c(c3)CN([C@H]3CCC(=O)NC3=O)C4=O)CC2)c(Cl)c1]

Step 1: To a mixture of 2-hydroxyethyl acetate (3.00 g, 28.8 mmol), 4-bromophenol (5.48 g, 31.7 mmol), and diphenyl-2-pyridylphosphine (8.00 g, 30.4 mmol) in toluene (60 mL) was added bis(2-methoxyethyl) azodicarboxylate (7.00 g, 29.9 mmol) at 0° C. The mixture was allowed to gradually warm to room temperature and stirred overnight. The mixture was diluted with EtOAc and washed with 3N aq. HCl (2 times) solution, washed with H₂O, washed with brine, dried over Na₂SO₄, filtered, and concentrated. The residue was purified by FCC on SiO₂ using a gradient of 10-100% CH₂Cl₂ in heptanes. 40a was collected as a colorless oil in 32% yield (2.36 g, 9.11 mmol). TLC (10% EtOAc/heptanes): R_{f}=0.22, TLC (30% EtOAc/heptanes): R_{f}=0.49, TLC (CH₂Cl₂): R_{f}=0.61. LC/MS found 259.0 [M+H]⁺.
Step 2: A mixture of 2-(4-bromophenoxy)ethyl acetate (527 mg, 2.03 mmol), tert-butyl 4-[2-chloro-4-(methylamino)phenyl]piperazine-1-carboxylate (689 mg, 2.11 mmol), methanesulfonato(2-dicyclohexylphosphino-2′,6′-di-i-propoxy-1,1′-biphenyl)(2′-methylamino-1,1′-biphenyl-2-yl)palladium(II) (43 mg, 51 μmol), 2-dicyclohexylphosphino-2′,6′-di-i-propoxy-1,1′-biphenyl (24 mg, 51 μmol), and cesium carbonate (1.34 g, 4.11 mmol) in dry 1,4-dioxane (10 mL) was degassed via sparging with N₂ for 10 minutes then heated to 80° C. with stirring for 4 hours. The mixture was cooled to room temperature and filtered through a pad of diatomaceous earth, and the filtrate was concentrated to dryness. The residue was purified by flash column chromatography using a gradient of 5-10% EtOAc in CH₂Cl₂. 40b was collected as a yellow oil with 39% purity in 38% yield (1.01 g, 0.78 mmol). TLC (30% EtOAc/hexanes): R_{f}=0.35, TLC (10% EtOAc/CH₂Cl₂): R_{f}=0.55. LC/MS found 504.3 [M+H]⁺.
Step 3: A mixture of tert-butyl 4-[4-[4-(2-acetoxyethoxy)-N-methyl-anilino]-2-chloro-phenyl]piperazine-1-carboxylate (1.01 g, 0.78 mmol) in THF (10 mL) and aq. sodium hydroxide (3 N, 30 mmol, 10 mL) solution was heated to 40° C. with stirring overnight. The mixture was cooled to room temperature, poured over H₂O, and extracted with EtOAc. The combined organic layers were washed with H₂O, washed with brine, dried over Na₂SO₄, filtered, and concentrated. The residue was purified by FCC on SiO₂ using a gradient of 30-60% EtOAc in heptanes. 40c was collected as an orange semi-solid in 42% yield over 2 steps (395 mg, 0.86 mmol). TLC (30% EtOAc/heptanes): R_{f}=0.12, TLC (50% EtOAc/heptanes): R_{f}=0.27. LC/MS found 462.3 [M+H]⁺.
Step 4: To a solution of tert-butyl 4-[2-chloro-4-[4-(2-hydroxyethoxy)-N-methyl-anilino]phenyl]piperazine-1-carboxylate (320 mg, 0.69 mmol) in ethyl acetate (7 mL) was added 2-iodoxybenzoic acid (603 mg, 2.15 mmol). The mixture was heated to 80° C. with stirring for 3 hours then cooled to room temperature. The mixture was filtered through a sintered glass funnel and the solid was washed with EtOAc (3×2 mL). The filtrate was concentrated, and the residue was purified by FCC on SiO₂ using a gradient of 10-50% EtOAc in heptanes. 40d was collected as a viscous red oil in 83% yield (265 mg, 0.58 mmol). TLC (50% EtOAc/heptanes): R_{f}=0.45, LC/MS found 460.3 [M+H]⁺.
Step 5: To a solution of tert-butyl 4-[2-chloro-4-[N-methyl-4-(2-oxoethoxy)anilino]phenyl]piperazine-1-carboxylate (178 mg, 0.39 mmol) in dichloromethane (4 mL) was added 2-aminoacetonitrile (43 mg, 0.77 mmol). The mixture was stirred at room temperature for 30 minutes. Sodium triacetoxyborohydride (163 mg, 0.77 mmol) was then added, and stirring was continued for 3 hours. The mixture was quenched 1N aq. NaOH solution and the layers were separated. The aqueous was extracted with CH₂Cl₂ and the combined organic layers were washed with H₂O, washed with brine, dried over MgSO₄, filtered, and concentrated. The residue was purified by FCC on SiO₂ using a gradient of 50-100% EtOAc in hexanes. 40e was collected as a pale orange semi-solid in 49% yield (94 mg, 0.19 mmol). TLC (50% EtOAc/heptanes): R_{f}=0.13, TLC (70% EtOAc/heptanes): R_{f}=0.32, TLC (EtOAc): R_{f}=0.62. LC/MS found 500.3 [M+H]⁺.
Step 6: To a solution of tert-butyl 4-[2-chloro-4-[4-[2-(cyanomethylamino)ethoxy]-N-methyl-anilino]phenyl]piperazine-1-carboxylate (103 mg, 0.21 mmol) and triethylamine (0.09 mL, 0.65 mmol) in dichloromethane (2 mL) cooled to 0° C. was added benzyl (2,5-dioxopyrrolidin-1-yl) carbonate (66 mg, 0.26 mmol). The mixture was warmed to room temperature and stirred for 1 hour. The mixture was poured over H₂O and extracted with CH₂Cl₂. The combined organic layers were washed with brine, dried over MgSO₄, filtered, and concentrated. The residue was purified by FCC on SiO₂ using a gradient of 10-50% EtOAc in heptanes. 40f was collected as a white semi-solid in 72% yield (94 mg, 0.15 mmol). TLC (30% EtOAc/heptanes): R_{f}=0.19, TLC (50% EtOAc/heptanes): R_{f}=0.43, TLC (10% EtOAc/CH₂Cl₂): R_{f}=0.58. LC/MS D.N.I.
Step 7: To a solution of tert-butyl 4-[4-[4-[2-[benzyloxycarbonyl(cyanomethyl)amino]ethoxy]-N-methyl-anilino]-2-chloro-phenyl]piperazine-1-carboxylate (94 mg, 0.15 mmol) in dichloromethane (2 mL) was added trifluoroacetic acid (1.0 mL, 13 mmol) and the mixture was stirred at room temperature for 1 hour. The mixture was poured over 10% aq. K₂CO₃ solution with brine, extracted with CH₂Cl₂. The combined organic layers were washed with brine, dried over MgSO₄, filtered, and concentrated 40g was collected as a white solid in 72% yield (57 mg, 0.11 mmol) and used without further purification. LC/MS found 534.3 [M+H]⁺.
Step 8: A mixture of benzyl N-[2-[4-(3-chloro-N-methyl-4-piperazin-1-yl-anilino)phenoxy]ethyl]-N-(cyanomethyl)carbamate (57 mg, 0.11 mmol), 2-chloro-N-[2-[(3 S)-2,6-dioxo-3-piperidyl]-1-oxo-isoindolin-5-yl]acetamide (38 mg, 0.11 mmol), and potassium carbonate (30 mg, 0.22 mmol) in DMF (2 mL) was heated to 70° C. with stirring for 2 hours then cooled to room temperature, poured over H₂O, and extracted with 10% i-PrOH in EtOAc. The combined organic layers were washed with H₂O (3 times), washed with brine, dried over MgSO₄, filtered, and concentrated. The residue was purified by FCC on SiO₂ using a gradient of 1-10% CH₃OH in EtOAc. 40h was collected as a white solid in 17% yield (15 mg, 18 μmol). LC/MS found 833.6 [M+H]⁺, 743.6 [M+H−Bn]⁺, and 417.3 [M+2H]²⁺.
Step 9: A solution of benzyl N-[2-[4-[3-chloro-4-[4-[2-[[2-[(3S)-2,6-dioxo-3-piperidyl]-1-oxo-isoindolin-5-yl]amino]-2-oxo-ethyl]piperazin-1-yl]-N-methyl-anilino]phenoxy]ethyl]-N-(cyanomethyl)carbamate (15 mg, 18 μmol) in trifluoroacetic acid (2.0 mL, 26 mmol) was stirred at 60° C. for 2 hours. The mixture was cooled to room temperature, poured over 10% aq. K₂CO₃/brine, and extracted with 20% i-PrOH in CH₂Cl₂. The combined organic layers were washed with brine, dried over MgSO₄, filtered, and concentrated. The residue was purified by reverse phase C-18 chromatography using a gradient of 5-95% CH₃CN in H₂O with 0.1% HCO₂H. 40 was collected as a white solid in 23% yield (3.2 mg, 4.2 μmol). LC/MS found 717.4 [M+H]⁺ and 359.3 [M+2H]²⁺.

### General Scheme 31

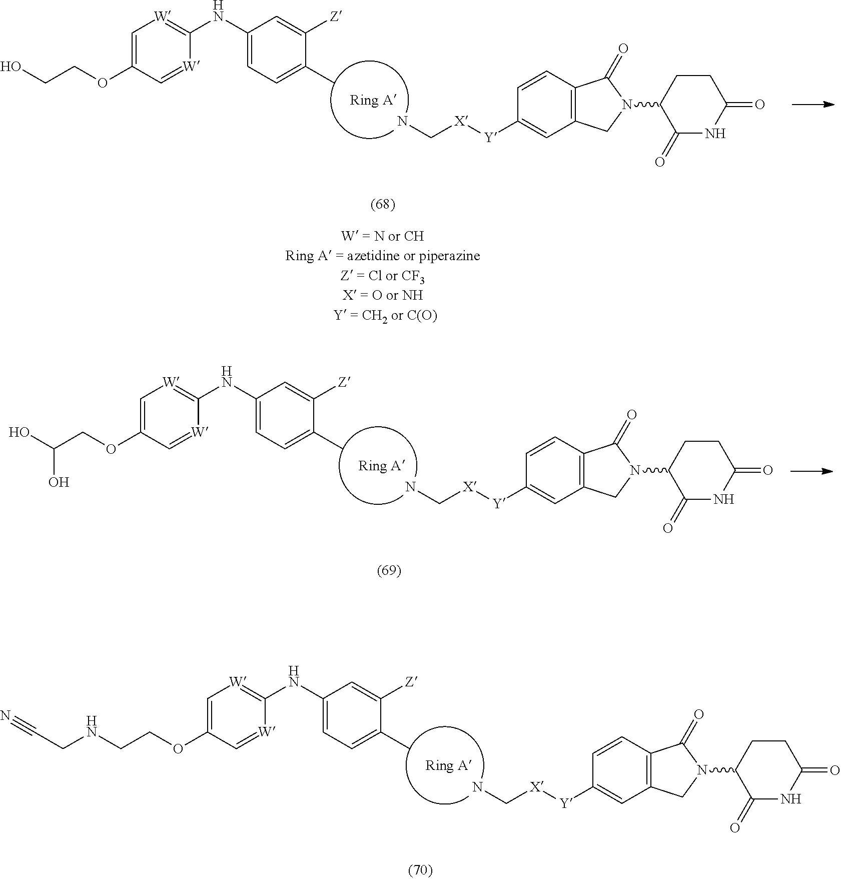

### Method 95

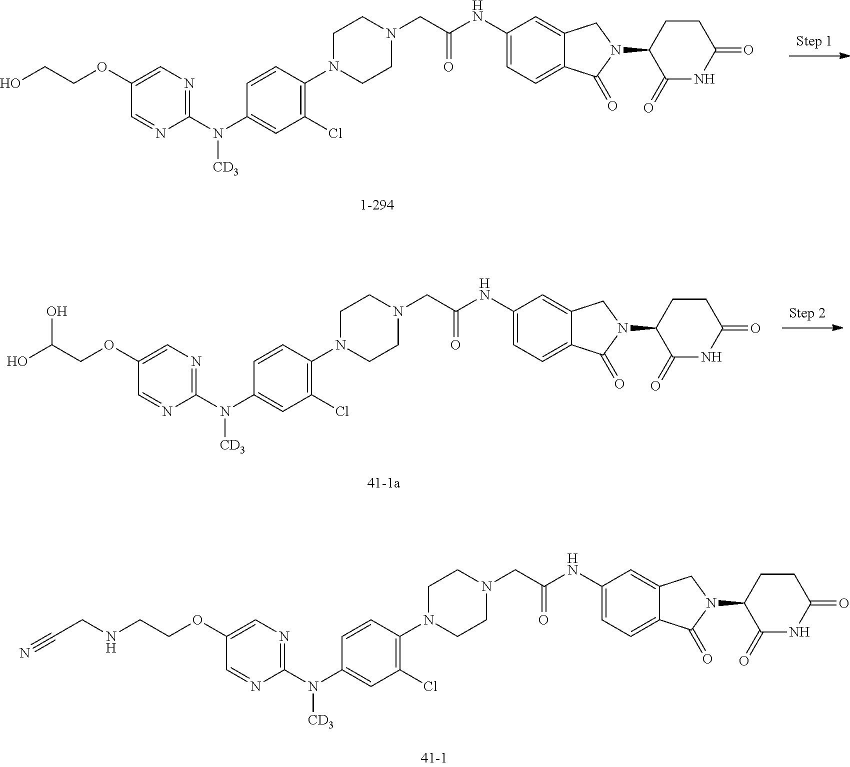 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCO)cn1 ; [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCC(O)O)cn1 ; [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCNCC#N)cn1]

Step 1: To a solution of 2-[4-[2-chloro-4-[[5-(2-hydroxyethoxy)pyrimidin-2-yl]-(trideuteriomethyl)amino]phenyl]piperazin-1-yl]-N-[2-[(3S)-2,6-dioxo-3-piperidyl]-1-oxo-isoindolin-5-yl]acetamide (1-294, 153 mg, 0.23 mmol) in acetone (10 mL) was added 2-iodoxybenzoic acid (193 mg, 0.69 mmol). The mixture was heated to 70° C. with stirring for 3 hours, then cooled to room temperature, filtered, and washed with a minimum of acetone. The filtrate was concentrated onto SiO₂ and purified by FCC on SiO₂ using a gradient of 1-10% CH₃OH in CH₂Cl₂. 41-1a was collected as a white solid in 60% yield (92 mg, 0.14 mmol). LC/MS found 682.5 [M+H+H₂O]⁺ and 341.6 [M+2H+H₂O]²⁺.
Step 2: To a solution of 2-[4-[2-chloro-4-[[5-(2-oxoethoxy)pyrimidin-2-yl]-(trideuteriomethyl)amino]phenyl]piperazin-1-yl]-N-[2-[(3S)-2,6-dioxo-3-piperidyl]-1-oxo-isoindolin-5-yl]acetamide (22 mg, 33 μmol) in dichloromethane (1 mL) and THF (1 mL) was added 2-aminoacetonitrile (6.4 mg, 0.11 mmol). The mixture was stirred at room temperature for 30 minutes then sodium triacetoxyborohydride (28 mg, 0.13 mmol) was added and stirring was continued overnight. The mixture was quenched with CH₃OH and evaporated on to SiO₂. The crude was purified by FCC on SiO₂ using a gradient of 1-10% CH₃OH in CH₂Cl₂. 41-1 was collected as a white solid with 94% purity in 26% yield (6.5 mg, 8.7 μmol). TLC (7% CH₃OH/CH₂Cl₂): R_{f}=0.31. LC/MS found 704.4 [M+H]⁺ and 352.8 [M+2H]²⁺.

The following compounds 41-1 through 41-3 (70) shown in Table 24 were prepared by methods similar to those described for the preparation of 41-1, using the appropriate intermediates (68).

TABLE 24

| Compd | Starting material | |
|---|---|---|
| | 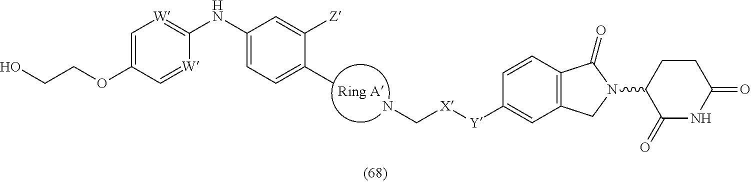 | |
| 41-1 | 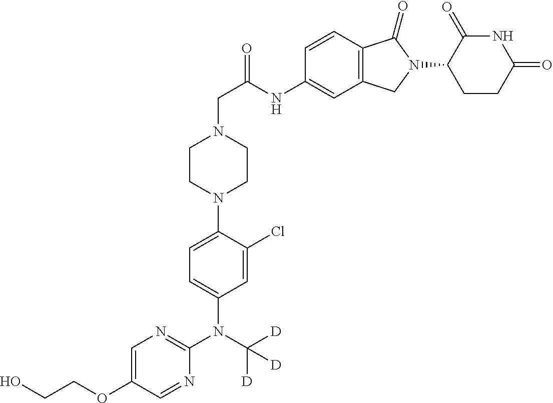 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCO)cn1] | |
| 41-2 | 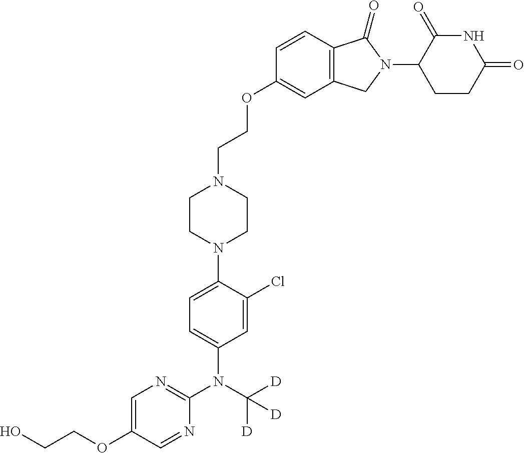 [SMILES: [2H]C([2H])([2H])N(c1ccc(N2CCN(CCOc3ccc4c(c3)CN(C3CCC(=O)NC3=O)C4=O)CC2)c(Cl)c1)c1ncc(OCCO)cn1] | |
| 41-3 | 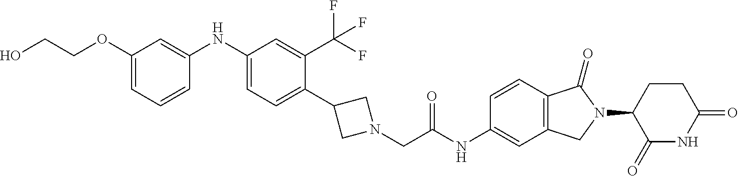 [SMILES: O=C1CC[C@H](N2Cc3cc(NC(=O)CN4CC(c5ccc(Nc6cccc(OCCO)c6)cc5C(F)(F)F)C4)ccc3C2=O)C(=O)N1] | |
| | | MS |
| | | found |
| | | [M + |
| Compd | Product | H]+ |
| | 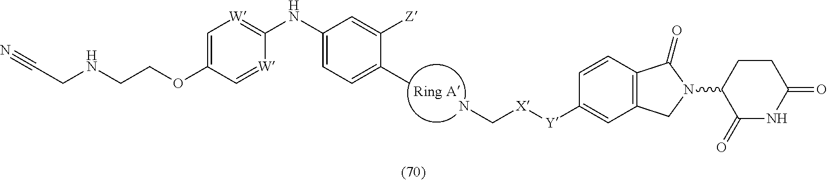 | |
| 41-1 | 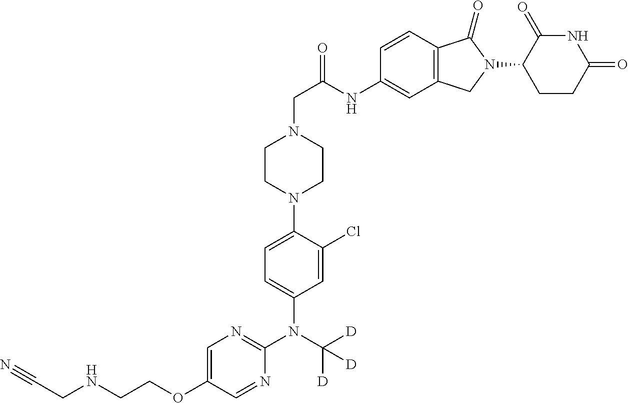 [SMILES: [2H]C([2H])([2H])N(c1ccc(N2CCN(CC(=O)Nc3ccc4c(c3)CN([C@H]3CCC(=O)NC3=O)C4=O)CC2)c(Cl)c1)c1ncc(OCCNCC#N)cn1] | 704.4 |
| 41-2 | 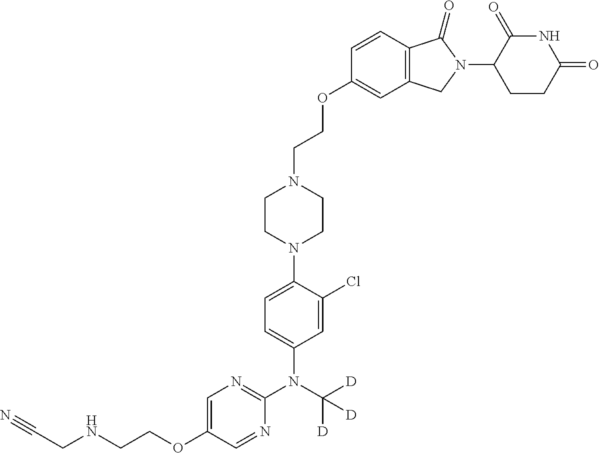 [SMILES: [2H]C([2H])([2H])N(c1ccc(N2CCN(CCOc3ccc4c(c3)CN(C3CCC(=O)NC3=O)C4=O)CC2)c(Cl)c1)c1ncc(OCCNCC#N)cn1] | 691.4 |
| 41-3 | 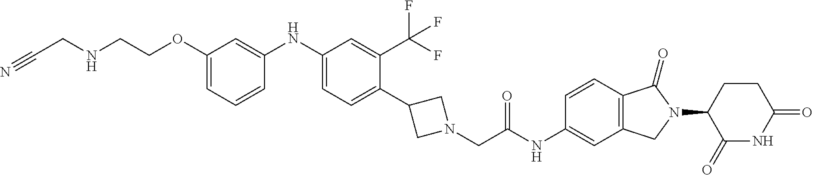 [SMILES: N#CCNCCOc1cccc(Nc2ccc(C3CN(CC(=O)Nc4ccc5c(c4)CN([C@H]4CCC(=O)NC4=O)C5=O)C3)c(C(F)(F)F)c2)c1] | 690.4 |

### Method 96

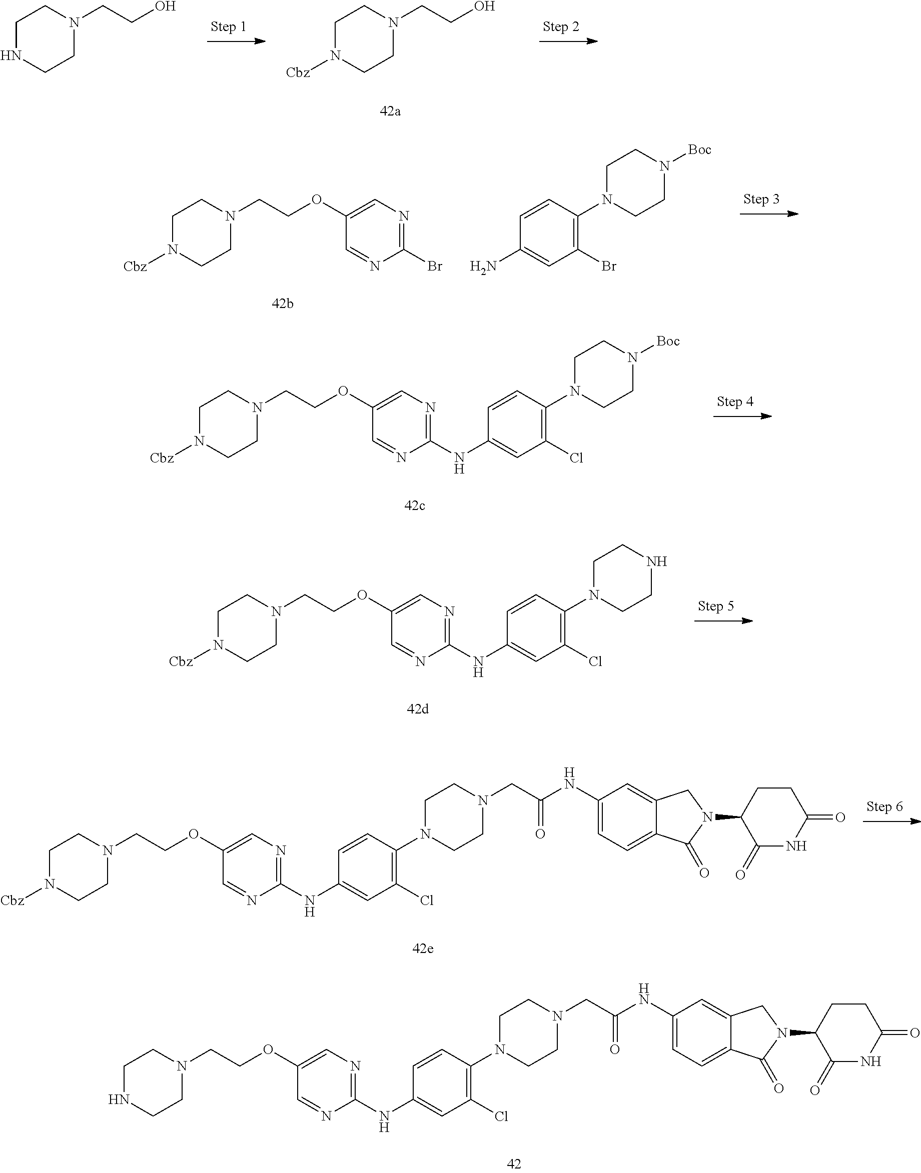

Step 1: To a solution of 2-piperazin-1-ylethanol (3.21 mL, 26.1 mmol) and triethylamine (9.0 mL, 65 mmol) in dichloromethane (28 mL) cooled to 0° C. was added dropwise benzyl carbonochloridate (3.0 mL, 21 mmol). The mixture was stirred for 1 hour then quenched with sat. aq. NaHCO₃ solution. The layers were separated and the aqueous was washed with H₂O, washed with brine, dried over Na₂SO₄, filtered, and concentrated. The residue was purified by FCC on SiO₂ using a gradient of 1-10% CH₃OH in CH₂Cl₂. 42a was collected as a colorless oil in 63% yield (3.53 g, 13.4 mmol). TLC (5% CH₃OH/CH₂Cl₂): R_{f}=0.20, TLC (10% CH₃OH/CH₂Cl₂): R_{f}=0.40, visualized with PMA stain. LC/MS found 265.4 [M+H]⁺.
Step 2: To a mixture of 2-bromopyrimidin-5-ol (1.28 g, 7.32 mmol), benzyl 4-(2-hydroxyethyl)piperazine-1-carboxylate (1.76 g, 6.66 mmol), and triphenylphosphine (1.84 g, 7.02 mmol) in toluene (8 mL) was added bis(2-methoxyethyl) azodicarboxylate (1.64 g, 6.99 mmol) at 0° C. The mixture was degassed via sparging with N₂ for 5 minutes and heated to 100° C. with stirring overnight. The mixture was poured over H₂O and extracted with EtOAc. The combined organic layers were washed with 1N aq. NaOH solution (2 times), washed with H₂O, washed with brine, dried over Na₂SO₄, filtered, and concentrated. The residue was purified by FCC on SiO₂ using a gradient of 50-100% EtOAc in heptanes. 42b was collected as a colorless viscous oil in 43% yield (1.20 g, 2.85 mmol). TLC (EtOAc): R_{f}=0.25. LC/MS found 421.2 [M+H]⁺.
Step 3: A mixture of benzyl 4-[2-(2-bromopyrimidin-5-yl)oxyethyl]piperazine-1-carboxylate (385 mg, 0.91 mmol), tert-butyl 4-(4-amino-2-chloro-phenyl)piperazine-1-carboxylate (300 mg, 0.96 mmol), methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II) (21 mg, 23 μmol), 2-(dicyclohexylphosphino)-3,6-dimethoxy-2′,4′,6′-tri-i-propyl-1,1′-biphenyl (12 mg, 22 μmol), and cesium carbonate (600 mg, 1.84 mmol) in dry 1,4-dioxane (5 mL) was degassed via sparging with N₂ for 10 minutes then heated to 80° C. with stirring for 4 hours. The mixture was cooled to room temperature and filtered through a pad of diatomaceous earth, and the filtrate was concentrated to dryness. The residue was purified by flash column chromatography using a gradient of 60-100% EtOAc in hexanes. 42c was collected as a light brown semi-solid in 92% yield (548 mg, 0.84 mmol). TLC (EtOAc): R_{f}=0.30. LC/MS found 652.4 [M+H]⁺.
Step 4: To a solution of benzyl 4-[2-[2-[4-(4-tert-butoxycarbonylpiperazin-1-yl)-3-chloro-anilino]pyrimidin-5-yl]oxyethyl]piperazine-1-carboxylate (274 mg, 0.42 mmol) in dichloromethane (3 mL) was added trifluoroacetic acid (1.0 mL, 13 mmol) and the mixture was stirred at room temperature for 1 hour. The mixture was poured over 1N aq. NaOH solution, extracted with CH₂Cl₂. The combined organic layers were washed with brine, dried over MgSO₄, filtered, and concentrated 42d was collected as an off-white in 68% yield (157 mg, 0.28 mmol) and used without further purification. LC/MS found 552.3 [M+H]⁺ and 276.8 [M+2H]²⁺.
Step 5: A mixture of benzyl 4-[2-[2-(3-chloro-4-piperazin-1-yl-anilino)pyrimidin-5-yl]oxyethyl]piperazine-1-carboxylate (157 mg, 0.28 mmol), 2-chloro-N-[2-[(3S)-2,6-dioxo-3-piperidyl]-1-oxo-isoindolin-5-yl]acetamide (100 mg, 0.30 mmol), and potassium carbonate (80 mg, 0.58 mmol) in DMF (2 mL) was heated to 70° C. with stirring for 2 hours then cooled to room temperature, poured over H₂O, and extracted with 10% i-PrOH in EtOAc. The combined organic layers were washed with H₂O (3 times), washed with brine, dried over MgSO₄, filtered, and concentrated. The residue was purified by FCC on SiO₂ using a gradient of 1-10% CH₃OH in CH₂Cl₂. 42e was collected as a white solid with 99% purity in 27% yield (67 mg, 78 μmol). TLC (7% CH₃OH/CH₂Cl₂): R_{f}=0.30, TLC (10% CH₃OH/CH₂Cl₂): R_{f}=0.44. LC/MS found 851.5 [M+H]⁺ and 426.4 [M+2H]²⁺.
Step 6: A solution of benzyl 4-[2-[2-[3-chloro-4-[4-[2-[[2-[(3S)-2,6-dioxo-3-piperidyl]-1-oxo-isoindolin-5-yl]amino]-2-oxo-ethyl]piperazin-1-yl]anilino]pyrimidin-5-yl]oxyethyl]piperazine-1-carboxylate (67 mg, 78 μmol) in trifluoroacetic acid (2.0 mL, 26 mmol) was stirred at 60° C. for 2 hours. The mixture was cooled to room temperature, poured over 10% aq. K₂CO₃/brine, and extracted with 20% i-PrOH in CH₂Cl₂. The combined organic layers were washed with brine, dried over MgSO₄, filtered, and concentrated. The residue was purified by reverse phase C-18 chromatography using a gradient of 5-95% CH₃CN in H₂O with 0.1% HCO₂H. 42 was collected as a white solid in 45% yield (27 mg, 35 μmol). LC/MS found 717.4 [M+H]⁺ and 359.3 [M+2H]²⁺.

### Method 97

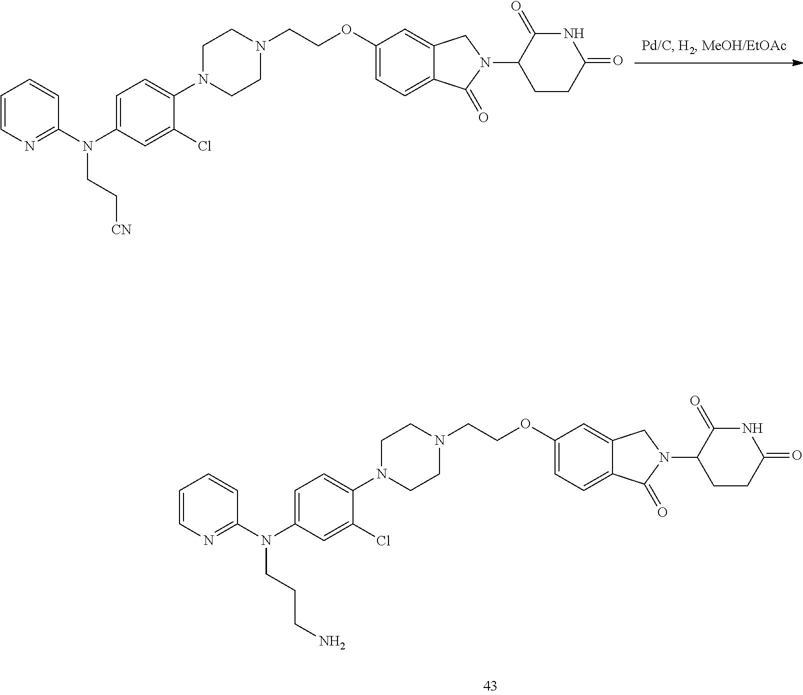 [SMILES: N#CCCN(c1ccc(N2CCN(CCOc3ccc4c(c3)CN(C3CCC(=O)NC3=O)C4=O)CC2)c(Cl)c1)c1ccccn1 ; NCCCN(c1ccc(N2CCN(CCOc3ccc4c(c3)CN(C3CCC(=O)NC3=O)C4=O)CC2)c(Cl)c1)c1ccccn1]

A solution of 3-[3-chloro-4-[4-[2-[2-(2,6-dioxo-3-piperidyl)-1-oxo-isoindolin-5-yl]oxyethyl]piperazin-1-yl]-N-(2-pyridyl)anilino]propanenitrile (50 mg, 79.60 μmmol), 10% Pd/C (16.94 mg, 159.20 μmol) in MeOH/EtOAc (1/1, 6 mL) was stirred at 25° C. for overnight under of H₂. The mixture was filtered, and the filtrate was concentrated to afford a crude product. The crude product was purified by silica gel chromatography (DCM/MeOH=10/1 to 5/1) to obtain title compound (43, 3.7 mg, 7.2% yield). ESI-MS: m/z=632.5, 634.5 [M+1]⁺.

### Method 98

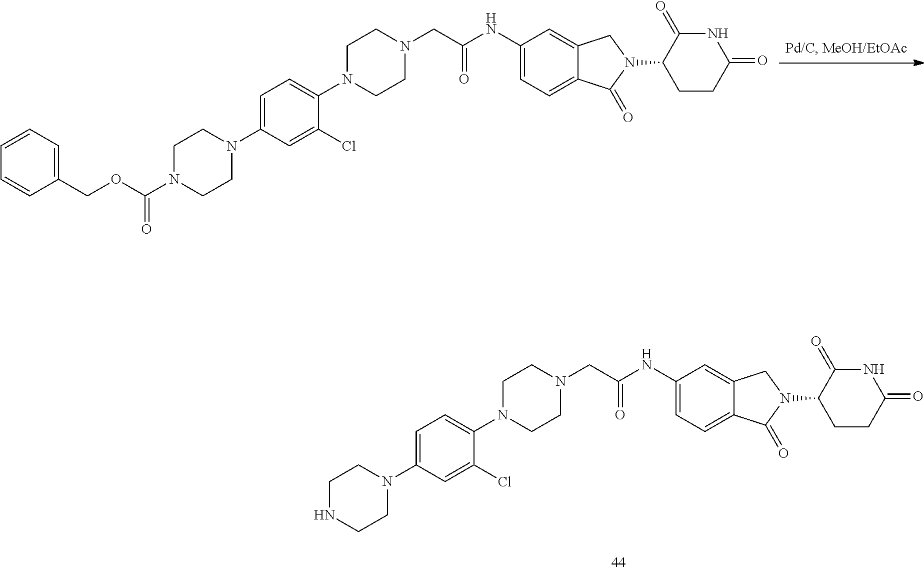 [SMILES: O=C1CC[C@H](N2Cc3cc(NC(=O)CN4CCN(c5ccc(N6CCN(C(=O)OCc7ccccc7)CC6)cc5Cl)CC4)ccc3C2=O)C(=O)N1 ; O=C1CC[C@H](N2Cc3cc(NC(=O)CN4CCN(c5ccc(N6CCNCC6)cc5Cl)CC4)ccc3C2=O)C(=O)N1]

A solution of benzyl 4-[3-chloro-4-[4-[2-[[2-[(3S)-2,6-dioxo-3-piperidyl]-1-oxo-isoindolin-5-yl]amino]-2-oxo-ethyl]piperazin-1-yl]phenyl]piperazine-1-carboxylate (130 mg, 182.02 μmmol), 10% Pd/C (19.37 mg, 182.02 μmol) in MeOH/EtOAc (1/1, 10 mL) was stirred at 25° C. for overnight under of H₂. The mixture was filtered, and the filtrate was concentrated to afford a crude product. The crude product was purified by silica gel chromatography (DCM/MeOH=20:1 to 5:1) to obtain title compound (44, 59.2 mg, 56.0% yield). ESI-MS: m/z=580.5, 582.4 [M+1]⁺.

### Example 2

### Evaluation of Compound Activity and Cell Viability Across Cancer Cell Line Panel

MATERIALS AND METHODS
A. Cell lines:

| | |
|---|---|
| SW948 | Colorectal adenocarcinoma |
| COLO201 | Colorectal adenocarcinoma |
| SW480 | Colorectal adenocarcinoma |
| NCIH747 | Colorectal adenocarcinoma |
| HCT116 | Colorectal adenocarcinoma |
| HCT15 | Colorectal adenocarcinoma |
| HepG2 | Hepatoblastoma |
| HepG2/C3A | Hepatoblastoma |
| HUH7 | Hepatocellular carcinoma |
| Hep3B | Hepatocellular carcinoma |
| SNU449 | Hepatocellular carcinoma |
| SK-HEP1 | Hepatocellular carcinoma |
| SNU387 | Hepatocellular carcinoma |
| SNU475 | Hepatocellular carcinoma |
| A549 | Non-Small Cell Lung Cancer |
| VERO | Monkey Kidney Epithelial cells |
| LK2 | Non-Small Cell Lung Cancer |
| HCC95 | Non-Small Cell Lung Cancer |
| H1155 | Non-Small Cell Lung Cancer |
| H520 | Non-Small Cell Lung Cancer |
| NCIH2009 | Non-Small Cell Lung Cancer |
| HCC15 | Non-Small Cell Lung Cancer |
| NCIH1299 | Non-Small Cell Lung Cancer |
| NCIH441 | Non-Small Cell Lung Cancer |
| NCIH460 | Non-Small Cell Lung Cancer |
| NCIH1975 | Non-Small Cell Lung Cancer |
| SW900 | Non-Small Cell Lung Cancer |
| NCIH1650 | Non-Small Cell Lung Cancer |

B. Cell culture medium, reagents, and equipment

RPMI1640 (#10-041 CV; Corning)
DMEM (#10-013CV; Corning)
Fetal bovine serum (#S11150H; R&D systems)
Penicillin/Streptomycin (#15140-122; GIBCO)
DMSO (#B159-4; Fisher Chemical)
TrypLE express (#12605-028; GIBCO)
Dulbecco's Phosphate Buffer Saline: DPBS (#14190-144; GIBCO)
Cell Titre-Glo (#G7573; Promega)
Two-step Luciferase (Firefly & Renilla) assay (#60683; BPS Bioscience)
Cell lysis buffer (#9803; Cell Signaling Technology)
Protease/Phosphatase inhibitor cocktail (#5872; Cell Signaling Technology)
Protein assay reagent (#1856209; Thermo Scientific)
Albumin standard (#23209; Thermo Scientific)
NuPage LDS sample buffer, 4X (#NP0007; Invitrogen)
NuPage sample reducing agent (#NP0009; Invitrogen)
NuPAGE MOPS SDS Running Buffer, (#NP000102; Thermo Fisher Scientific)
NuPAGE ™ Bis-Tris Mini Protein Gels, 4-12%, 1.0-1.5 mm #NP0321BOX
(Invitrogen)
NuPAGE ™ Bis-Tris Midi Protein Gels, 4 to 12%, 1.0 mm #WG1403BOX
(Invitrogen)
Prestained protein ladder, Chameleon Duo (#928-60000, Li-Cor)
Transblot Turbo transfer buffer (#10026938; Biorad)
Primary antibodies from Cell Signaling Technology - anti-c-myc (#5605) and
anti-n-myc (#51705) or ProteinTech - anti-GSPT1 (#68271) anti-GSPT2 (#12989-
1-AP), anti-β-actin (#66009), Helios (#36426). Santa Cruz Biotechnology -
Ikaros (#sc-398265), Proteintech - Aiolos (#19055-1-AP)
Secondary antibodies from Li-Cor, goat anti-mouse-IR dye800-CW 926-32210,
goat anti-rabbit-IR dye 800-CW (#926-32211), goat anti-mouse-IR dye680-
RD (#926-68070), goat anti-rabbit-IR dye 680-DR (#926-68071).
Blocking non-specific antibody binding and for incubation with primary or
secondary antibodies, Li-Cor Intercept (TBS) Blocking Buffer and Antibody
Diluent Kit, (#927-66003) was utilized.
Washing buffer, 20X Tris buffered saline with Tween (#J77500-K2, Lot#231309,
ThermoScientific)
Western blot Imaging: Li-Cor Odessey DLx imaging system
TCF/LEF-Luciferase reporter (#79787, BPS Bioscience)
MYC-Luciferase reporter (#60519, BPS Bioscience)
Renilla-Luciferase (#79565, BPS Bioscience)
96 well plates (#3903; Costar, #3606; Costar, #9018; Costar)
6 well plates (#3505; Costar)
10 cm dishes (#664160; Griener)
15 mL conical centrifuge tubes (#352096; Falcon)
50 mL conical centrifuge tubes (#352070; Falcon)
Serological pipettes 5 mL (#170355N; Thermo scientific) 10 mL (#170356N;
Thermo scientific) & 25 mL (#13-678-11; Fisher scientific).
Tips purchased from USA Scientific 10 μL (#111-3800), 200 μL (#1110-1200) &
1000 μL (#1126-7810)
VWR Ergonomic High Performance pipettes single channel (P2.5, P10, P20,
P100, P200 & P1000) and 12 well - multichannel (P50 & P300).
Repeater pipette, E1 ClipTip (Thermo scientific)
12.5 μL tips ClipTip (#94420043, Thermoscientific), 125 μL ClipTips (#4423,
Integra), 1250 μL ClipTips (#4444, Integra biosciences)
Pipette basin/reservoir (#RES-V-50-5; Axygen)
Sterile Cell lifter (#3008; Corning)
Plate readers - SpectraMax Plus 384 (Molecular Devices) and BioTek Synergy
HT with BioStack 3 microplate stacker.
Incubators - Thermo Electron Corp, Stericycle CO₂ incubator, HEPA Class 100
and HERACell V10S150i.
Biological safety cabinets - Labcono Class II, Type A2
Gel apparatus: Invitrogen PowerEase Touch 350 precast gel system, (#EI8675,
Invitrogen)
Transfer apparatus: Transblot Turbo Transfer System and starter kit (#170019157,
Biorad)
Membrane: Immobilon-FL (#IPFL00010, Millipore)
VWR standard analog shaker
VWR vistavison inverted microscope
Nexcelom cellometer AutoT4, Cell counting chamber (#SD100)
VWR L010 −20° C. freezer
Thermoscientific TSX series −80° C. freezer
VWR analog heatblock
Labnet Prism R, refrigerated microcentrifuge
Sorvall ST plus centrifuge (Thermofisher)
Refrigerated centrifuge 5810R (Eppendorf)
Mettler Toledo Balance ME203TE

The working stock of compounds was prepared by 1:10 dilution of the original 10 mM stock using DMSO (#B159-4; Fisher Chemical) to a final concentration of 1 mM. Using working stock, a master compound plate consisting of 100× compounds with a 10-point dilution (corresponding to final concentrations of 0-1000 nM) was prepared using a 3-fold dilution series by combining 1 part of the previously diluted compound with 2 parts of DMSO (#B159-4; Fisher Chemical). By using a multichannel pipette, 1 μL of the diluted compound was dispensed from each row of the master plate into 96 well tissue culture treated plates (#3903; Costar), in triplicate.

To evaluate the effect of compounds on cell viability, cell lines were counted and adjusted to 5×10⁴/ml, and 100 μL of cells were dispensed per well. The plates were then returned to a CO₂ incubator for 72 hours and maintained at 37° C. The cell viability was assessed on day 3 by measuring intracellular ATP content with CellTitre-Glo (#G7573; Promega) and using BioTek Synergy HT plate reader. Graphpad Prism software was used to calculate the growth inhibitory activity (IC₅₀) values based on a four-parameter non-linear regression curve fitting algorithm and visualize the dose response effect for each compound treatment and cell line based on percent cell viability.

The compounds of Formula (1-1) exhibited potent pharmacological properties. As used herein, the term “growth-inhibitory activity” (IC₅₀, nM) refers to the concentration required to inhibit cell growth by 50%. The results are shown in Table 25 below, where the degree of growth inhibition is categorized as follows: IC₅₀<11 nM=A; 11-50 nM=B; 51-500 nM=C; >500 nM=D; and ND=not determined. Table 25 illustrates representative growth inhibition data for CRBN modulator, exemplified by compounds of Formula (1-1).

TABLE 25
Growth Inhibition Activity of CRBN modulator

| Compound | LK2 IC₅₀ | HUH7 IC₅₀ | COLO201 IC₅₀ |
|---|---|---|---|
| 1-1 | D | D | ND |
| 1-2 | C | B | ND |
| 1-3 | C | B | ND |
| 1-4 | A | B | ND |
| 1-5 | A | A | ND |
| 1-6 | A | A | ND |
| 1-7 | B | A | ND |
| 1-8 | B | B | ND |
| 1-9 | B | A | A |
| 1-10 | B | B | ND |
| 1-11 | A | A | A |
| 1-12 | A | A | ND |
| 1-13 | A | A | ND |
| 1-14 | D | ND | ND |
| 1-15 | A | A | ND |
| 1-16 | D | ND | ND |
| 1-17 | B | D | ND |
| 1-18 | A | A | ND |
| 1-19 | A | B | ND |
| 1-20 | D | ND | ND |
| 1-21 | D | ND | ND |
| 1-22 | D | B | ND |
| 1-23 | B | B | B |
| 1-24 | D | ND | ND |
| 1-25 | D | ND | ND |
| 1-26 | D | ND | ND |
| 1-27 | B | A | A |
| 1-28 | B | A | A |
| 1-29 | B | A | ND |
| 1-30 | A | A | ND |
| 1-31 | B | A | ND |
| 1-32 | A | A | ND |
| 1-33 | A | A | ND |
| 1-34 | A | A | ND |
| 1-35 | A | A | ND |
| 1-36 | C | B | B |
| 1-37 | A | A | ND |
| 1-38 | A | A | A |
| 1-39 | A | A | ND |
| 1-40 | B | A | ND |
| 1-41 | A | A | B |
| 1-42 | A | B | C |
| 1-43 | B | A | A |
| 1-44 | A | B | ND |
| 1-45 | A | A | ND |
| 1-46 | A | A | ND |
| 1-47 | D | ND | ND |
| 1-48 | B | A | ND |
| 1-49 | B | B | ND |
| 1-50 | C | A | A |
| 1-51 | A | A | ND |
| 1-52 | A | A | ND |
| 1-53 | A | A | ND |
| 1-54 | A | A | A |
| 1-55 | B | A | ND |
| 1-56 | D | ND | ND |
| 1-57 | B | B | B |
| 1-58 | A | A | ND |
| 1-59 | A | A | ND |
| 1-60 | A | A | ND |
| 1-61 | A | A | ND |
| 1-62 | A | A | ND |
| 1-63 | B | A | B |
| 1-64 | B | A | B |
| 1-65 | B | A | B |
| 1-66 | A | A | B |
| 1-67 | A | A | C |
| 1-68 | C | B | A |
| 1-69 | B | A | B |
| 1-70 | A | A | A |
| 1-71 | A | A | A |
| 1-72 | A | A | A |
| 1-73 | A | A | A |
| 1-74 | A | A | A |
| 1-75 | B | ND | ND |
| 1-76 | A | A | A |
| 1-77 | A | A | A |
| 1-78 | A | A | A |
| 1-79 | A | A | A |
| 1-80 | A | A | A |
| 1-81 | D | ND | ND |
| 1-82 | D | ND | ND |
| 1-83 | C | B | C |
| 1-84 | A | ND | A |
| 1-85 | A | A | A |
| 1-86 | D | ND | ND |
| 1-87 | D | ND | ND |
| 1-88 | B | ND | ND |
| 1-89 | B | ND | ND |
| 1-90 | A | A | A |
| 1-91 | B | ND | ND |
| 1-92 | B | ND | ND |
| 1-93 | B | ND | ND |
| 1-94 | B | ND | ND |
| 1-95 | B | A | A |
| 1-96 | A | A | A |
| 1-97 | A | A | A |
| 1-98 | A | A | A |
| 1-99 | B | ND | B |
| 1-100 | B | ND | ND |
| 1-101 | A | A | A |
| 1-102 | A | A | A |
| 1-103 | B | ND | ND |
| 1-104 | A | A | A |
| 1-105 | A | A | A |
| 1-106 | D | ND | ND |
| 1-107 | A | ND | A |
| 1-108 | A | ND | ND |
| 1-109 | A | A | A |
| 1-110 | A | A | A |
| 1-111 | A | A | A |
| 1-112 | B | ND | ND |
| 1-113 | A | A | A |
| 1-114 | A | A | A |
| 1-115 | B | ND | B |
| 1-116 | A | A | A |
| 1-117 | D | ND | ND |
| 1-118 | A | A | A |
| 1-119 | A | A | A |
| 1-120 | A | A | A |
| 1-121 | B | ND | ND |
| 1-122 | A | A | A |
| 1-123 | A | A | A |
| 1-124 | B | ND | ND |
| 1-125 | A | A | A |
| 1-126 | A | A | A |
| 1-127 | C | A | B |
| 1-128 | B | A | A |
| 1-129 | A | A | A |
| 1-130 | A | A | A |
| 1-131 | B | ND | ND |
| 1-132 | B | ND | ND |
| 1-133 | A | A | A |
| 1-134 | A | A | A |
| 1-135 | B | ND | ND |
| 1-136 | A | A | A |
| 1-137 | A | A | A |
| 1-138 | C | ND | ND |
| 1-139 | B | ND | ND |
| 1-140 | B | A | ND |
| 1-141 | A | A | A |
| 1-142 | B | ND | ND |
| 1-143 | A | A | A |
| 1-144 | B | ND | ND |
| 1-145 | B | ND | ND |
| 1-146 | A | A | A |
| 1-147 | A | A | A |
| 1-148 | A | A | A |
| 1-149 | B | ND | ND |
| 1-150 | A | A | A |
| 1-151 | C | ND | C |
| 1-152 | B | ND | C |
| 1-153 | A | A | A |
| 1-154 | A | A | A |
| 1-155 | A | A | A |
| 1-156 | A | A | A |
| 1-157 | A | A | A |
| 1-158 | A | A | A |
| 1-159 | A | A | A |
| 1-160 | A | A | A |
| 1-161 | A | A | A |
| 1-162 | A | A | A |
| 1-163 | A | A | A |
| 1-164 | B | ND | ND |
| 1-165 | A | A | A |
| 1-166 | A | A | A |
| 1-167 | A | A | A |
| 1-168 | A | A | A |
| 1-169 | A | A | B |
| 1-170 | A | A | A |
| 1-171 | A | A | A |
| 1-172 | A | A | A |
| 1-173 | A | A | A |
| 1-174 | A | A | A |
| 1-175 | A | ND | ND |
| 1-176 | A | ND | ND |
| 1-177 | B | ND | ND |
| 1-178 | A | ND | ND |
| 1-179 | A | ND | ND |
| 1-180 | B | A | A |
| 1-181 | A | ND | ND |
| 1-182 | A | ND | ND |
| 1-183 | A | A | A |
| 1-184 | A | A | A |
| 1-185 | A | A | A |
| 1-186 | A | A | A |
| 1-187 | B | A | A |
| 1-188 | A | A | A |
| 1-189 | A | A | A |
| 1-190 | A | A | A |
| 1-191 | A | A | A |
| 1-192 | B | A | A |
| 1-193 | A | A | A |
| 1-194 | A | A | A |
| 1-195 | C | B | B |
| 1-196 | A | A | A |
| 1-197 | B | A | A |
| 1-198 | A | A | A |
| 1-199 | A | A | A |
| 1-200 | A | A | B |
| 1-201 | A | A | B |
| 1-202 | A | A | A |
| 1-203 | C | C | C |
| 1-204 | A | A | A |
| 1-205 | B | A | C |
| 1-206 | A | A | A |
| 1-207 | A | A | B |
| 1-208 | B | A | B |
| 1-209 | C | C | C |
| 1-210 | C | B | C |
| 1-211 | A | A | A |
| 1-212 | A | A | A |
| 1-213 | A | A | A |
| 1-214 | A | A | A |
| 1-215 | A | A | A |
| 1-216 | A | A | A |
| 1-217 | A | A | A |
| 1-218 | A | A | A |
| 1-219 | A | A | A |
| 1-220 | D | D | D |
| 1-221 | B | A | B |
| 1-222 | C | B | C |
| 1-223 | D | C | D |
| 1-224 | B | B | B |
| 1-225 | A | A | A |
| 1-226 | B | A | A |
| 1-227 | A | A | A |
| 1-228 | A | A | A |
| 1-229 | A | A | A |
| 1-230 | B | A | A |
| 1-231 | B | A | A |
| 1-232 | B | B | B |
| 1-233 | B | B | B |
| 1-234 | B | A | B |
| 1-235 | B | A | B |
| 1-236 | A | A | A |
| 1-237 | A | A | A |
| 1-238 | D | ND | ND |
| 1-239 | D | ND | ND |
| 1-240 | C | C | ND |
| 1-241 | C | C | ND |
| 1-241 | A | A | ND |
| 1-246 | A | A | ND |
| 1-247 | B | A | ND |
| 1-248 | A | A | ND |
| 1-249 | ND | ND | ND |
| 1-250 | ND | ND | ND |
| 1-251 | ND | ND | ND |
| 1-252 | ND | ND | ND |
| 1-253 | A | A | A |
| 1-254 | A | A | A |
| 1-255 | C | C | C |
| 1-256 | A | A | A |
| 1-257 | A | A | B |
| 1-258 | A | A | A |
| 1-259 | B | A | C |
| 1-260 | ND | ND | ND |
| 1-261 | B | A | B |
| 1-262 | D | B | D |
| 1-263 | A | A | A |
| 1-264 | A | A | ND |
| 1-265 | A | A | ND |
| 1-266 | C | D | ND |
| 1-267 | ND | ND | ND |
| 1-268 | ND | ND | ND |
| 1-269 | D | D | D |
| 1-269 | B | A | B |
| 1-270 | B | B | B |
| 1-271 | D | D | D |
| 1-272 | A | B | ND |
| 1-273 | D | D | ND |
| 1-274 | B | B | ND |
| 1-275 | D | D | ND |
| 1-276 | A | B | ND |
| 1-277 | D | D | ND |
| 1-278 | A | A | ND |
| 1-280 | B | B | ND |
| 1-281 | B | D | ND |
| 1-282 | ND | ND | ND |
| 1-284 | D | D | ND |
| 1-285 | ND | ND | ND |
| 1-287 | B | A | ND |
| 1-288 | A | A | ND |
| 1-289 | ND | ND | ND |
| 1-290 | ND | ND | ND |
| 1-291 | ND | ND | ND |
| 1-292 | ND | ND | ND |
| 1-293 | ND | ND | ND |
| 1-294 | ND | ND | ND |
| 2-1 | D | ND | ND |
| 2-2 | D | ND | ND |
| 2-3 | D | ND | D |
| 2-4 | A | A | B |
| 2-5 | D | C | D |
| 2-6 | B | A | B |
| 2-7 | D | ND | ND |
| 2-8 | A | A | A |
| 2-9 | A | A | A |
| 2-10 | A | A | A |
| 2-11 | A | A | A |
| 2-12 | A | ND | A |
| 2-13 | A | A | A |
| 2-14 | A | A | A |
| 2-15 | A | A | A |
| 2-16 | A | A | A |
| 2-17 | A | A | A |
| 2-18 | B | B | B |
| 2-19 | B | A | B |
| 2-20 | D | ND | ND |
| 2-21 | A | A | A |
| 2-22 | A | A | A |
| 2-23 | A | A | A |
| 2-24 | A | A | A |
| 2-25 | A | A | A |
| 2-26 | A | A | A |
| 2-27 | A | A | A |
| 2-28 | A | A | A |
| 2-29 | A | A | A |
| 2-30 | A | A | A |
| 2-31 | A | A | A |
| 2-32 | A | A | A |
| 2-33 | A | A | A |
| 2-34 | C | B | C |
| 2-35 | A | A | A |
| 2-36 | A | A | A |
| 2-37 | A | A | A |
| 2-38 | A | A | A |
| 2-39 | A | A | A |
| 2-40 | B | A | A |
| 2-41 | B | A | A |
| 2-42 | A | A | A |
| 2-43 | A | A | B |
| 2-44 | A | A | A |
| 2-45 | D | C | D |
| 2-46 | A | A | A |
| 2-47 | C | C | C |
| 2-48 | C | C | C |
| 2-49 | B | A | B |
| 2-50 | A | A | A |
| 2-51 | A | A | A |
| 2-52 | B | A | B |
| 2-53 | B | A | B |
| 2-54 | A | A | A |
| 2-55 | A | A | A |
| 2-56 | A | A | A |
| 2-57 | A | A | A |
| 2-58 | A | A | A |
| 2-59 | A | A | A |
| 2-60 | A | A | A |
| 2-61 | C | C | C |
| 2-62 | A | A | A |
| 2-63 | B | A | B |
| 2-64 | A | A | A |
| 2-65 | A | A | A |
| 2-66 | A | A | A |
| 2-67 | A | A | A |
| 2-68 | B | A | B |
| 2-69 | A | A | A |
| 2-70 | A | A | A |
| 2-71 | A | A | A |
| 2-72 | A | A | A |
| 2-73 | A | A | A |
| 2-74 | A | A | A |
| 2-75 | A | A | A |
| 2-76 | A | A | A |
| 2-77 | B | A | C |
| 2-78 | A | A | A |
| 2-79 | A | A | A |
| 2-80 | B | A | B |
| 2-81 | B | A | B |
| 2-82 | B | B | B |
| 2-83 | D | D | D |
| 2-84 | B | B | B |
| 2-85 | D | B | C |
| 2-86 | D | D | D |
| 2-87 | D | D | D |
| 2-88 | D | D | D |
| 2-89 | D | B | D |
| 2-90 | D | B | C |
| 2-91 | D | C | D |
| 2-92 | B | A | B |
| 2-93 | D | B | D |
| 2-94 | D | D | D |
| 2-95 | B | A | B |
| 2-96 | D | B | D |
| 2-97 | D | A | D |
| 2-98 | D | D | D |
| 2-99 | B | A | B |
| 2-100 | D | B | D |
| 2-101 | D | C | D |
| 2-102 | C | B | C |
| 2-103 | B | B | B |
| 2-104 | B | B | B |
| 2-105 | B | B | B |
| 2-106 | C | C | C |
| 2-107 | C | B | C |
| 2-108 | D | B | D |
| 2-109 | B | B | C |
| 2-110 | B | B | C |
| 2-111 | B | B | B |
| 2-112 | D | D | D |
| 2-113 | A | A | A |
| 2-114 | D | D | D |
| 2-115 | D | D | D |
| 2-116 | D | D | D |
| 2-117 | D | D | D |
| 2-118 | D | D | D |
| 2-119 | A | A | A |
| 2-120 | A | A | A |
| 2-121 | D | C | D |
| 2-122 | B | A | ND |
| 2-123 | B | B | B |
| 2-124 | C | B | C |
| 2-125 | B | B | B |
| 2-126 | A | A | A |
| 2-127 | A | A | A |
| 2-128 | A | A | A |
| 2-129 | A | A | A |
| 2-130 | B | B | B |
| 2-131 | D | D | D |
| 2-132 | D | D | D |
| 2-133 | D | D | D |
| 2-134 | D | D | D |
| 2-135 | D | D | D |
| 2-136 | A | A | A |
| 2-137 | A | A | A |
| 2-138 | A | A | A |
| 2-139 | A | A | A |
| 2-140 | B | A | B |
| 2-141 | A | A | A |
| 2-142 | A | A | A |
| 2-143 | A | A | A |
| 2-144 | A | A | A |
| 2-145 | A | A | A |
| 2-146 | C | B | A |
| 2-147 | D | D | D |
| 2-148 | B | A | A |
| 2-149 | D | D | D |
| 2-150 | D | D | D |
| 2-151 | D | D | D |
| 2-152 | D | D | D |
| 2-153 | D | D | D |
| 2-154 | D | D | D |
| 2-155 | ND | A | ND |
| 2-156 | A | A | ND |
| 2-158 | A | A | ND |
| 2-159 | A | A | ND |
| 2-160 | A | A | ND |
| 2-161 | A | A | ND |
| 2-162 | ND | A | ND |
| 2-163 | ND | ND | ND |
| 2-164 | ND | ND | ND |
| 2-165 | ND | ND | ND |
| 2-166 | D | B | D |
| 2-168 | A | A | A |
| 2-170 | ND | ND | ND |
| 2-172 | B | B | B |
| 2-173 | D | D | D |
| 2-174 | C | C | C |
| 2-175 | A | A | A |
| 2-176 | D | B | ND |
| 2-177 | D | B | ND |
| 2-178 | D | D | ND |
| 2-179 | D | D | ND |
| 2-180 | A | C | ND |
| 2-181 | ND | ND | ND |
| 2-182 | ND | ND | ND |
| 2-183 | ND | ND | ND |
| 2-184 | ND | ND | ND |
| 2-185 | ND | ND | ND |
| 2-186 | ND | ND | ND |
| 2-187 | ND | ND | ND |
| 3-1 | B | A | B |
| 3-2 | D | ND | ND |
| 3-3 | A | A | A |
| 3-4 | A | A | D |
| 3-5 | B | A | B |
| 3-6 | B | A | B |
| 4-1 | D | ND | ND |
| 4-2 | D | ND | ND |
| 4-3 | C | C | C |
| 4-4 | C | C | C |
| 5-1 | D | D | D |
| 5-2 | D | D | D |
| 6-1 | B | C | B |
| 6-2 | D | C | C |
| 6-3 | C | C | ND |
| 6-4 | D | C | ND |
| 6-5 | D | D | ND |
| 6-6 | D | D | ND |
| 6-7 | D | D | ND |
| 6-8 | D | C | ND |
| 6-9 | D | C | ND |
| 6-11 | D | C | ND |
| 6-12 | D | D | ND |
| 6-13 | B | C | ND |
| 6-14 | B | B | ND |
| 6-15 | B | B | ND |
| 6-16 | B | C | ND |
| 6-17 | D | D | ND |
| 6-18 | B | C | ND |
| 6-19 | A | B | ND |
| 6-20 | C | C | ND |
| 6-21 | D | D | ND |
| 6-22 | D | D | ND |
| 6-23 | D | D | ND |
| 7-1 | D | B | C |
| 7-3 | C | B | ND |
| 7-4 | A | A | ND |
| 7-5 | A | A | ND |
| 7-6 | A | A | ND |
| 7-7 | A | B | ND |
| 7-8 | B | B | ND |
| 7-9 | B | A | ND |
| 7-10 | A | A | ND |
| 7-11 | A | A | ND |
| 7-12 | A | A | ND |
| 7-13 | C | A | ND |
| 7-14 | B | A | ND |
| 7-15 | C | A | ND |
| 7-16 | B | B | ND |
| 7-17 | B | B | ND |
| 7-18 | A | B | ND |
| 7-19 | B | B | ND |
| 7-20 | A | B | ND |
| 7-21 | B | C | ND |
| 7-22 | B | B | ND |
| 7-23 | B | A | ND |
| 7-24 | B | A | ND |
| 7-25 | A | A | ND |
| 7-26 | B | B | ND |
| 7-27 | B | B | ND |
| 7-28 | B | B | ND |
| 7-29 | B | A | ND |
| 7-30 | D | D | ND |
| 7-31 | A | A | ND |
| 7-32 | A | A | ND |
| 7-33 | A | A | ND |
| 7-34 | A | A | ND |
| 7-35 | C | A | ND |
| 7-36 | D | B | ND |
| 7-37 | ND | ND | ND |
| 7-38 | D | B | ND |
| 7-39 | C | C | ND |
| 7-40 | C | B | ND |
| 7-41 | D | C | ND |
| 7-42 | ND | ND | ND |
| 7-43 | ND | ND | ND |
| 7-44 | ND | ND | ND |
| 7-45 | B | A | ND |
| 7-46 | ND | ND | ND |
| 7-47 | B | B | ND |
| 7-48 | B | B | ND |
| 7-49 | C | B | ND |
| 7-50 | B | B | ND |
| 7-51 | A | A | ND |
| 7-52 | C | C | ND |
| 7-53 | B | A | ND |
| 7-54 | B | A | ND |
| 7-55 | A | D | ND |
| 7-56 | B | A | ND |
| 7-57 | C | A | ND |
| 7-58 | C | A | ND |
| 7-59 | B | A | ND |
| 7-60 | B | B | ND |
| 7-61 | C | A | ND |
| 7-62 | B | A | ND |
| 7-63 | ND | ND | ND |
| 7-64 | ND | ND | ND |
| 7-65 | ND | ND | ND |
| 7-66 | ND | ND | ND |
| 7-67 | ND | ND | ND |
| 7-68 | ND | ND | ND |
| 7-69 | ND | ND | ND |
| 7-70 | ND | ND | ND |
| 7-71 | ND | ND | ND |
| 7-72 | ND | ND | ND |
| 7-73 | ND | ND | ND |
| 7-74 | ND | ND | ND |
| 7-75 | ND | ND | ND |
| 7-76 | ND | ND | ND |
| 7-77 | ND | ND | ND |
| 7-78 | ND | ND | ND |
| 7-79 | ND | ND | ND |
| 7-80 | ND | ND | ND |
| 7-81 | ND | ND | ND |
| 7-82 | D | D | D |
| 7-83 | B | A | ND |
| 7-84 | B | A | ND |
| 7-85 | B | A | ND |
| 7-86 | A | B | ND |
| 7-87 | B | B | ND |
| 7-88 | D | D | ND |
| 7-89 | B | C | ND |
| 7-90 | D | C | ND |
| 7-91 | D | D | ND |
| 7-93 | B | B | ND |
| 7-94 | A | A | ND |
| 7-95 | B | A | ND |
| 7-96 | B | B | ND |
| 7-97 | C | B | ND |
| 7-98 | A | A | ND |
| 7-99 | ND | ND | ND |
| 7-100 | ND | ND | ND |
| 7-101 | ND | ND | ND |
| 7-102 | ND | ND | ND |
| 7-103 | ND | ND | ND |
| 7-104 | ND | ND | ND |
| 7-105 | ND | ND | ND |
| 7-106 | ND | ND | ND |
| 7-107 | ND | ND | ND |
| 7-108 | ND | ND | ND |
| 7-109 | ND | ND | ND |
| 7-110 | ND | ND | ND |
| 7-111 | ND | ND | ND |
| 7-112 | ND | ND | ND |
| 7-113 | ND | ND | ND |
| 7-114 | ND | ND | ND |
| 7-115 | ND | ND | ND |
| 7-116 | ND | ND | ND |
| 7-117 | ND | ND | ND |
| 8-1 | C | B | ND |
| 8-2 | D | D | ND |
| 8-3 | D | D | ND |
| 8-4 | C | C | ND |
| 8-5 | B | B | ND |
| 8-6 | B | B | ND |
| 8-7 | A | B | ND |
| 8-8 | A | C | ND |
| 8-9 | B | A | ND |
| 8-10 | A | B | ND |
| 8-11 | D | D | ND |
| 8-12 | D | D | ND |
| 8-13 | A | B | ND |
| 8-14 | A | B | ND |
| 9-1 | D | D | ND |
| 9-2 | A | A | ND |
| 9-3 | D | D | ND |
| 9-4 | A | A | ND |
| 9-5 | A | A | ND |
| 9-6 | A | A | |
| 9-7 | A | A | ND |
| 9-8 | A | A | ND |
| 9-9 | B | A | ND |
| 9-10 | ND | ND | ND |
| 9-11 | ND | ND | ND |
| 9-12 | ND | ND | ND |
| 9-13 | ND | ND | ND |
| 9-14 | A | A | ND |
| 9-15 | ND | ND | ND |
| 10-1 | A | A | ND |
| 11-1 | D | D | ND |
| 11-2 | B | B | ND |
| 11-3 | B | B | ND |
| 11-4 | A | A | ND |
| 12-1 | A | A | ND |
| 12-2 | A | A | ND |
| 12-3 | A | A | ND |
| 12-4 | A | A | ND |
| 13-1 | C | C | ND |
| 13-2 | A | B | ND |
| 13-3 | A | B | ND |
| 13-4 | A | B | ND |
| 14 | C | C | ND |
| 14a | A | A | ND |
| 15 | D | D | ND |
| 16-1 | A | A | ND |
| 16-2 | A | A | ND |
| 16-3 | D | D | ND |
| 16-4 | A | A | ND |
| 16-5 | B | A | ND |
| 16-6 | B | A | ND |
| 16-7 | B | A | ND |
| 17-1 | D | D | D |
| 17-2 | B | D | ND |
| 17-3 | B | B | ND |
| 17-4 | B | A | ND |
| 17-5 | D | D | ND |
| 18-1 | A | A | ND |
| 18-2 | A | A | ND |
| 18-3 | B | A | ND |
| 18-4 | ND | ND | ND |
| 19-1 | A | A | ND |
| 19-2 | B | B | ND |
| 19-3 | B | A | ND |
| 19-4 | B | A | ND |
| 20-1 | A | A | ND |
| 20-2 | A | A | ND |
| 20-3 | A | A | ND |
| 20-4 | B | A | ND |
| 20-5 | ND | ND | ND |
| 20-6 | ND | ND | ND |
| 21-1 | C | C | ND |
| 21-2 | C | B | ND |
| 22-1 | ND | ND | ND |
| 22-2 | ND | ND | ND |
| 22-3 | ND | ND | ND |
| 23-1 | ND | ND | ND |
| 23-2 | ND | ND | ND |
| 23-3 | ND | ND | ND |
| 23-4 | ND | ND | ND |
| 23-5 | ND | ND | ND |
| 23-6 | ND | ND | ND |
| 24-1 | ND | ND | ND |
| 24-2 | ND | ND | ND |
| 25-1 | ND | ND | ND |
| 26 | C | C | C |
| 27 | D | D | ND |
| 28 | ND | ND | ND |
| 29 | D | D | D |
| 31 | D | C | C |
| 33 | D | D | D |
| 34 | ND | A | ND |
| 35 | ND | ND | ND |
| 36 | D | D | ND |
| 37 | ND | A | ND |
| 38-1 | ND | ND | ND |
| 38-2 | ND | ND | ND |
| 39-1 | ND | ND | ND |
| 39-2 | ND | ND | ND |
| 39-3 | ND | ND | ND |
| 39-4 | ND | ND | ND |
| 39-5 | ND | ND | ND |
| 41-1 | ND | ND | ND |
| 41-2 | ND | ND | ND |
| 41-3 | ND | ND | ND |
| 42 | ND | ND | ND |
| 43 | D | D | D |
| 44 | D | D | D |

### Example 3

### Proteomic Profiling of Compound-Treated Cancer Cells Reveals Degradation of GSPT1 and GSPT2

For proteomic profiling, 5×10⁶ cells (LK2 or HUH7 or H1155) were plated in 10 cm dishes consisting of complete RPMI medium (RPMI1640 (10-041 CV; Corning), 10% fetal bovine serum (S11150H; R&D systems), 1× Penicillin/Streptomycin (15140-122; GIBCO). 24 hours after plating the cells were treated with compounds, by dispensing 1 μL/mL of pre-diluted (100 μM) compound to result in 100 nM final concentration. Cell pellets were collected at 2 or 8 hours, scrapped into medium, centrifuged using 15 mL tubes, washed with ice-cold Dulbecco's Phosphate Buffer Saline (DPBS, #14190-144; GIBCO) 1 mL, transferred to 1.5 mL microcentrifuge tube, centrifuged to remove buffer completely and cell pellet was frozen in −80° C. until shipment in dry ice for proteomics. MS Bioworks processed and analyzed results as described further. Cells were lysed in 8M urea, 50 mM Tris HCl, pH 8.0, 1× Roche Complete Protease Inhibitor. Samples were sonicated using an amplitude of 35-40% with 1 second pulses on and off for 20 seconds. Lysates were clarified by centrifugation at 14000 g, and supernatants transferred to a new tube. Protein quantification was performed using Qubit. 50 μg of each sample was digested overnight with trypsin. Briefly, samples were reduced for 1 hour at room temperature in 12 mM Dithiothreitol (DTT) followed by alkylation for 1 hour at room temperature in 15 mM iodoacetamide. Trypsin was added to an enzyme:substrate ratio of 1:20. Each sample was acidified to 0.3% Trifluoroacetic acid (TFA) and subjected to solid phase extraction (SPE) using Waters pHLB elution plate Samples were then frozen at −80° C., lyophilized and reconstituted in 140 mM HEPES, pH 8.0, 30% acetonitrile for TMT labeling. 40 μL of ACN was added to each TMT tag tube and mixed aggressively. Tags were incubated at room temperature for 15 minutes. 30 μL of label was added to each peptide sample and mixed aggressively. Samples were incubated in an Eppendorf Thermomixer at 300 rpm 25° C. for 1.5 hours. Reactions were terminated with the addition of 8 μL of fresh 5% hydroxylamine solution and 15 minutes incubation at room temperature. 1% of each labeled sample was combined into a single 6-plex, lyophilized, and desalted using SPE. This was analyzed on a single 4-hour gradient and acquired using SPS-MS3. 50% was subjected to high pH reverse phase fractionation as follows: Buffers: Buffer A: 10 mM NH₄OH, pH 10.5, Water Buffer B: 10 mM NH₄OH, pH 10.5, ACN Column: XBridge C18, 2.1 mm ID×150 mm length, 3.5 μm particle size (Waters, part #186003023) HPLC system: Agilent 1100 equipped with a 150 μL sample loop operating at 0.3 mL/min, detector set at 214 nm wavelength. Dried peptides were resolubilized in 150 μL of Buffer A and injected manually. Fractions were collected every 30 seconds from 1 to 49 minutes (96 fractions total, 150 μL/fraction). Peptides (25% per pool) were analyzed by nano LC/MS/MS/MS using a Waters NanoAcquity HPLC system interfaced to a ThermoFisher Fusion Lumos mass spectrometer. Peptides were loaded on a trapping column and eluted over a 75 μm analytical column at 350 nL/min; both columns were packed with Luna C18 resin (Phenomenex). Each high pH RP pool was separated over a 1.5-hour gradient (36 hours instrument time total). The mass spectrometer was operated using MS2 method. MS scans were acquired in the Orbitrap at 120,000 FWHM resolution, MS2 scans were acquired in the Orbitrap using HCD at 35% NCE and 50,000 FWHM resolution. The isolation window was adjusted based on the charge state of the precursor. A 2-second cycle time was used. Data was processed through the MaxQuant software v1.6.14.0. Reporter ion intensities were Log₂ transformed. Normalized values were then subtracted from the median values (Treatment-Control) to obtain Log₂ foldchange. Student's t-test was performed comparing (Treatment vs. Control) with Benjamini-Hochberg FDR correction. Visualization as volcano plot was performed as XY scatter plot by using Log₂ foldchange and −Log₁₀ p-values from each dataset.

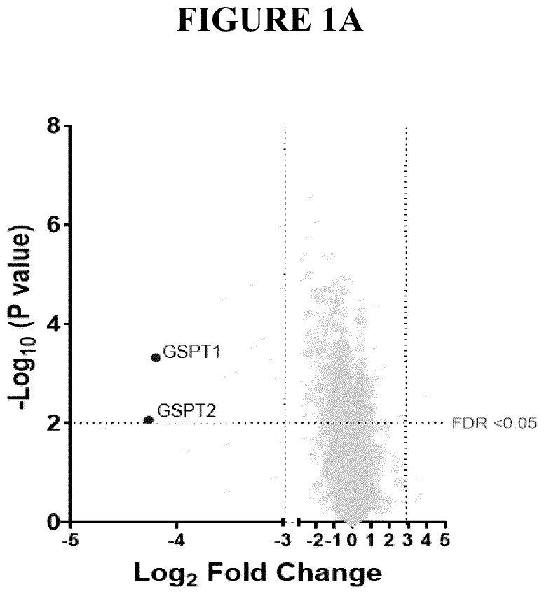

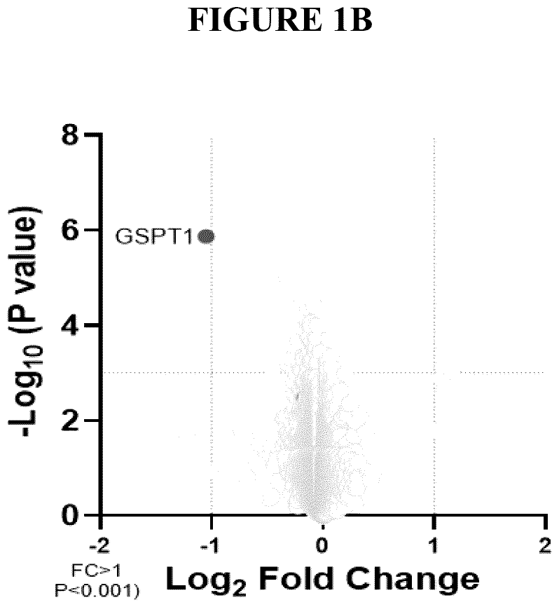

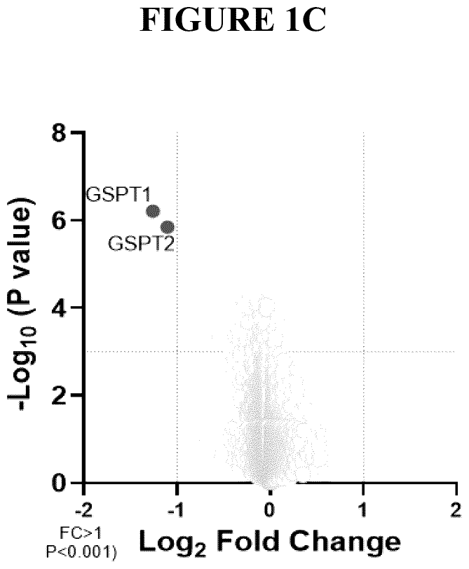

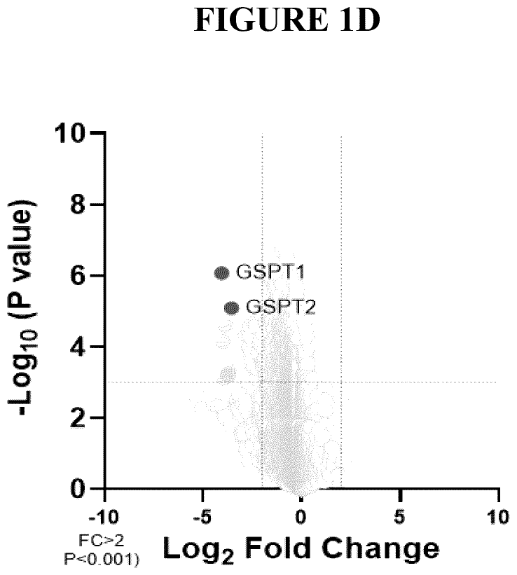

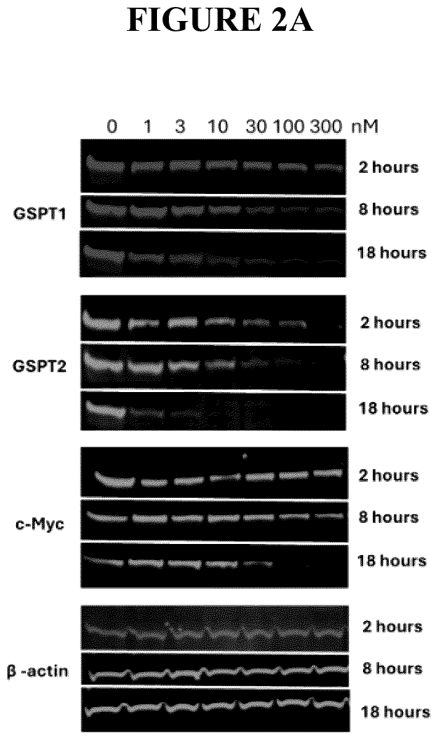

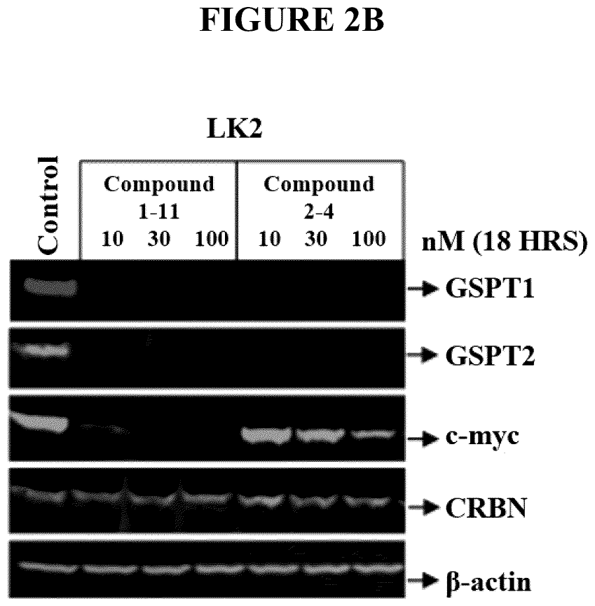

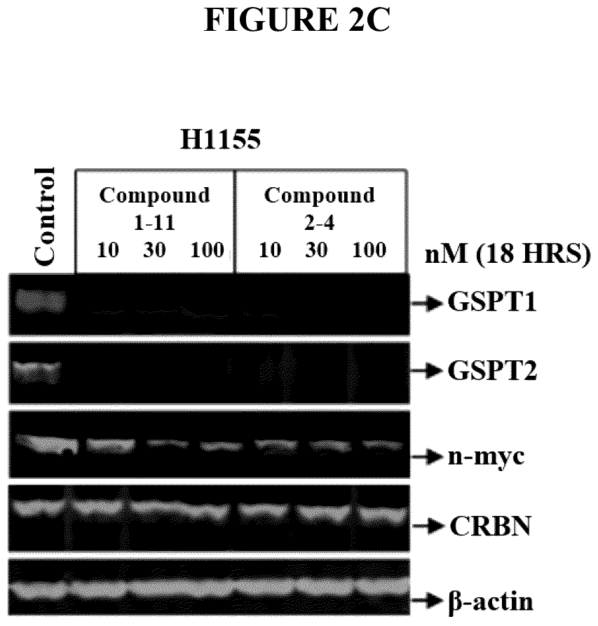

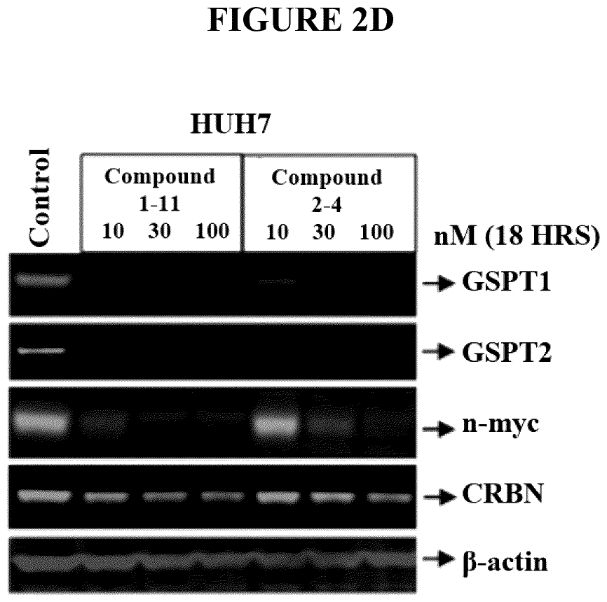

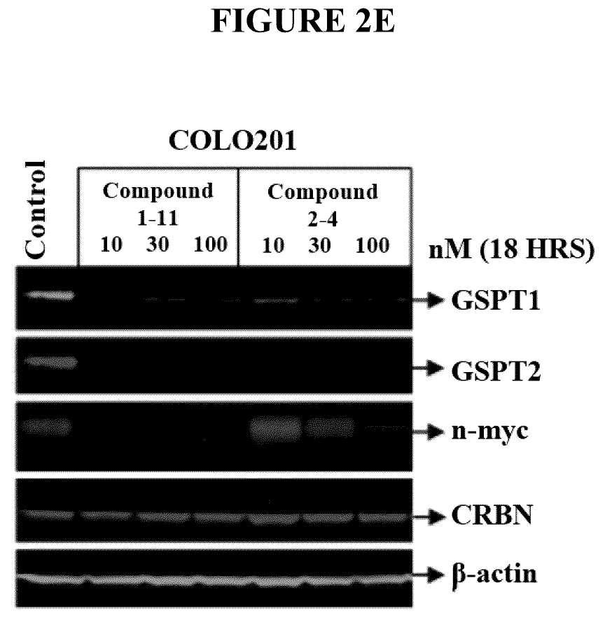

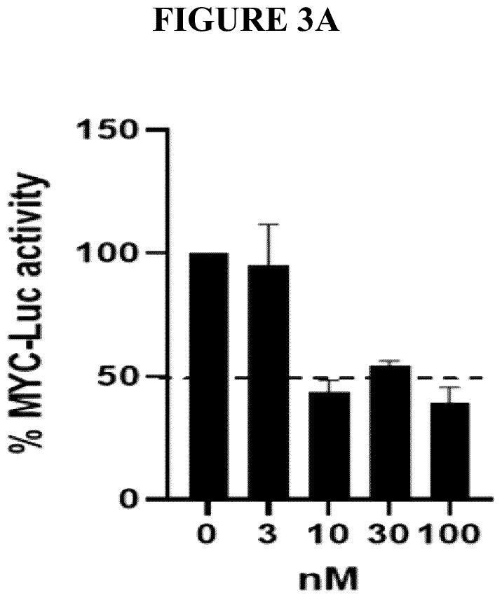

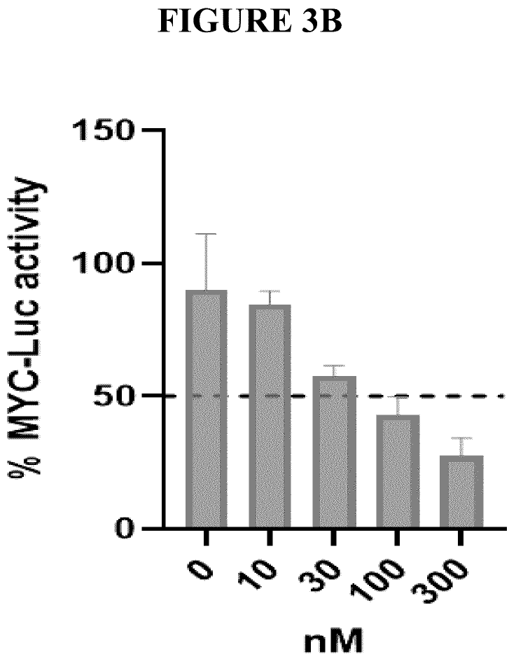

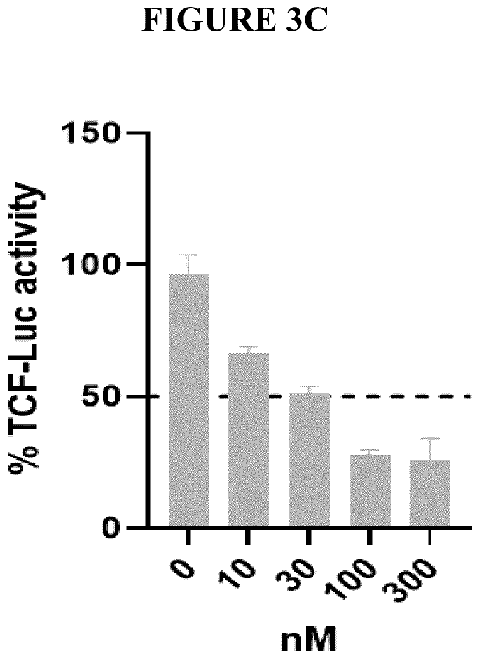

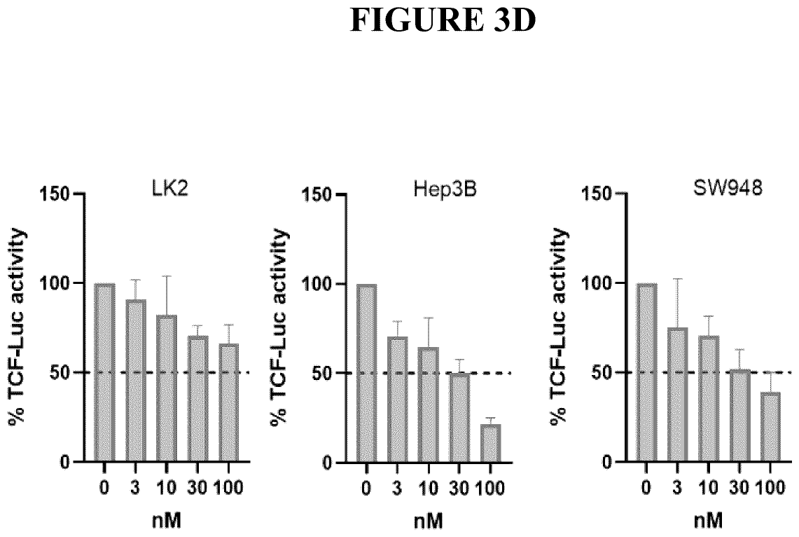

Overall, results from proteomic profiling experiments with lung and hepatocellular cancer cell lines, as shown in FIGS. 1A to 1D, demonstrate that both compounds 1-11 and 2-4 treatment resulted in significant GSPT1 protein degradation, without any notable non-specific or CRBN neo-substrate degradation.

### Example 4

### Western Blot Analysis of GSPT1, GSPT2, and Myc Degradation in Lung and Hepatic Cancer Cells Treated with Compounds

Lung squamous cell carcinoma cell lines, LK2 and H1155, and colon adenocarcinoma cell line COLO201 were cultured in RPMI-1640 medium supplemented with 10% heat inactivated fetal bovine serum, 100 U/mL penicillin and 100 μg/mL streptomycin at 37° C. in an atmosphere of 5% CO₂ in air. Hepatocellular cancer cell line HUH7 was cultured in DMEM medium supplemented with 10% heat inactivated fetal bovine serum, 100 U/mL penicillin and 100 μg/mL streptomycin at 37° C. in an atmosphere of 5% CO₂ in air. For time course kinetics, LK2 cells were treated with compound 2-4 (0-300 nM) for 2, 8 & 18 hours. For comparative analysis of compounds 1-11 and 2-4, LK2, H1155, HUH7 and COLO201 were treated with 10, 30, and 100 nM for 18 hours. Following treatment, cells were harvested by washing with ice cold phosphate buffered saline and lysing with 1× Cell Lysis Buffer (#98035, Lot #83, Cell Signaling Technology) consisting of protease/phosphatase inhibitor cocktail (#5872S, Lot #22, Cell Signaling Technology). Cell lysates were incubated on ice for 15 minutes, followed by centrifugation at 14,000 rpm for 15 minutes at 4° C., using Labnet Prism R, refrigerated microcentrifuge. Supernatants were collected and protein estimation was performed using Bradford Protein assay reagent (#1856209; Lot #YG371621, Thermo Scientific) and albumin standard (#23209; Lot #YH372319, Thermo Scientific) following manufacturer recommended protocols using 96 well clear bottom plates (#9018; Lot #3006001, Costar) and SpectraMax Plus 384 (Molecular Devices) plate reader. Protein concentrations were adjusted to 1 μg/μL with cell lysis buffer consisting of 1× gel LDS gel loading dye and reducing agent (NuPage LDS sample buffer, 4× (#NP0007; Lot #2201446, Invitrogen and NuPage sample reducing agent #NP0009; Lot #2565356, Invitrogen). Samples were then heated using VWR heat block, at 70° C. for 10 minutes and then chilled on ice. Equal amounts of protein (15-20 μg) were loaded on NuPAGE™ Bis-Tris Mini Protein Gels, 4-12% (#NP0321 or #WG1403BOX, Invitrogen), along with pre-stained protein ladder (Chameleon Duo #928-60000, Lot #D20323-04, Li-Cor). Protein separation was performed using Invitrogen PowerEase Touch 350 precast gel system, (#EI8675, Invitrogen), at 100 volt for 90 minutes, using prechilled NuPAGE MOPS SDS Running Buffer, (#NP000102; Thermo Fisher Scientific). Following protein separation, transfer of protein to methanol activated Immobilon-FL (#IPFL00010, Millipore) was performed at 120 volt and 12 minutes, using Transblot Turbo Transfer System (#170019157, Biorad) and Transblot Turbo transfer buffer (#10026938; Biorad). Following protein transfer, the blotted membrane was then washed and blocked for non-specific binding using Li-Cor Intercept (TBS) Blocking Buffer (#927-66003, Li-Cor) for 1 hour and then incubated overnight at 4° C. with appropriate primary antibody diluted in Intercept Antibody Diluent, (#927-66003, Li-Cor) Following primary antibodies from Cell Signaling Technology—anti-c-myc (#5605) and anti-n-myc (#51705) or ProteinTech—anti-GSPT1 (#68271) anti-GSPT2 (#12989-1-AP), anti-β-actin (#66009) were used in the western blot analysis. Blots were then washed three times, for 5 minutes with 1× Tris buffered saline with Tween (#J77500-K2, Lot #231309, ThermoScientific) and incubated for one hour at room temperature, with appropriate secondary antibodies purchased from Li-Cor, goat anti-mouse-IR dye800-CW 926-32210, goat anti-rabbit-IR dye 800-CW (#926-32211), goat anti-mouse-IR dye680-RD (#926-68070), goat anti-rabbit-IR dye 680-DR (#926-68071) at 1:10,000 dilution using Intercept Antibody Diluent, (#927-66003, Li-Cor). Blots were then washed three times, for 5 minutes with 1× Tris buffered saline with Tween (#J77500-K2, Lot #231309, ThermoScientific), washed once with 1× Tris buffered saline and were scanned using Li-Cor Odessey DLx imaging system.

### Example 5

### Effects of Compounds on Myc and TCF4/LEF Reporter Activity in Lung Squamous Cell Carcinoma Cells

Stable LK2 cells expressing MYC reporter-Luciferase and Renilla-Luciferase or TCF4/LEF reporter-Luciferase and Renilla-Luciferase were used in this experiment. TCF4/LEF represents WNT/β-Catenin signaling pathway, which has been known to regulate MYC protein expression and functions in cancers. Both MYC and WNT signaling pathways are known to be influenced by GSPT1. On Day 0, 90 μL consisting of 3000 LK2 reporter cells/well in complete RPMI1640 were dispensed into each well of 96 well white clear bottom plate (#3903; Costar). On Day 1, working stock of compounds was prepared by 1:10 dilution of the original 10 mM stock using DMSO (#B159-4; Fisher Chemical) to a final concentration of 1 mM. Using working stock, a 10× master compound plate consisting of compounds with a 5-point dilution (corresponding to final concentrations of 0-300 nM) was prepared using a 3-fold dilution series by combining 1 part of the previously diluted compound with 2 parts of DMSO (#B159-4; Fisher Chemical), then volumes were adjusted with cell culture medium to yield 10× concentration while consisting final 1% DMSO concentration. By using a multichannel pipette, 10 μL of the diluted compound was dispensed into 96 well plates consisting of cells, in triplicate. Plates were then incubated for 16 hours using CO₂ incubator maintained at 37° C. On Day 2, following incubation, plates were removed and 50 μL Dual Glo reagent (Promega Dual Glo Assay reagent kit; Cat #E2920) was added, incubated 30 minutes and plates were read luminometrically, using BioTek Synergy HT plate reader. Then 50 μL Stop and Glo reagent (reconstituted with substrate 1:100) (Promega Dual Glo Assay reagent kit; Cat #E2920) was added and plate was read luminometrically, using BioTek Synergy HT plate reader after 30 minutes. Results from reporter luciferase (Dual Glo) were normalized by dividing with Renilla-luciferase (Stop and glo) and percent reporter activity inhibition was calculated based on DMSO treated control wells. Graphpad Prism software was used to visualize the dose response effect based on the percent reporter activity calculated from these experiments.

### Example 6

### In Vivo Efficacy of Compound Treatment in Tumor Xenograft Models

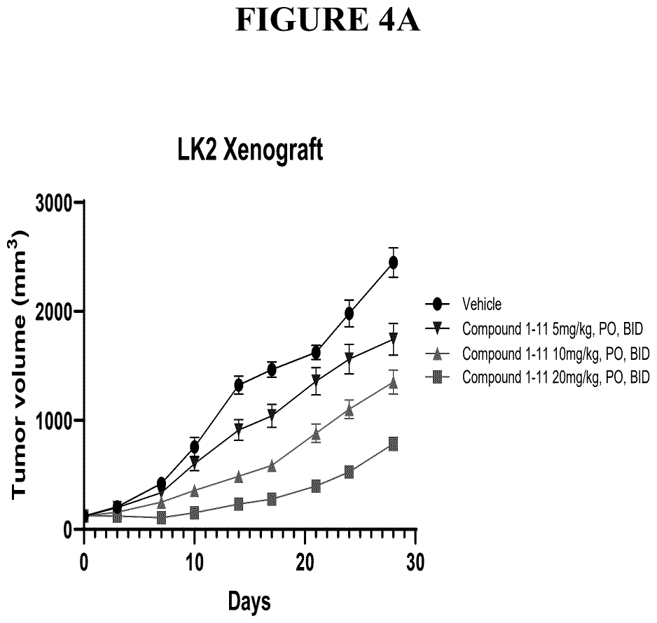

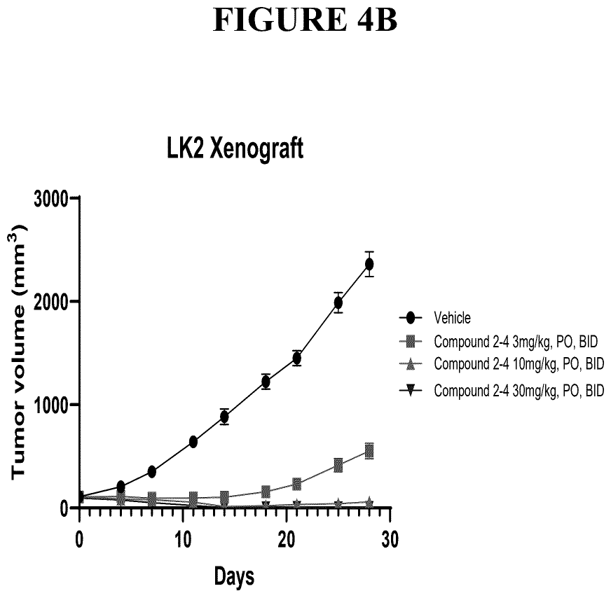

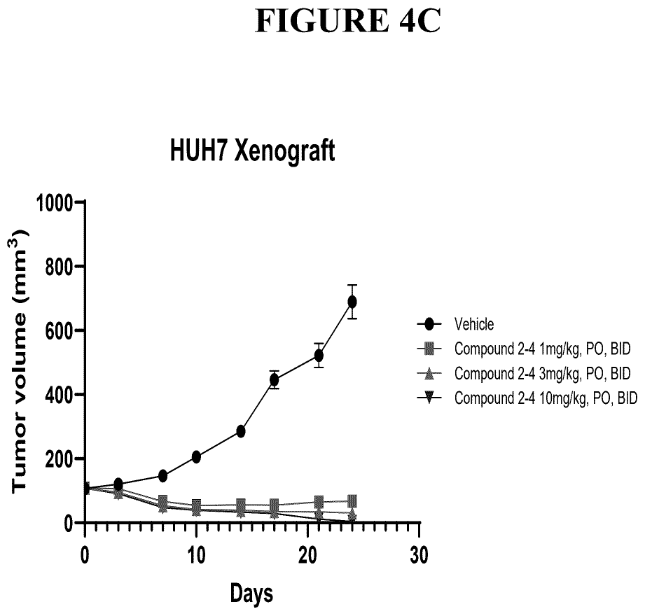

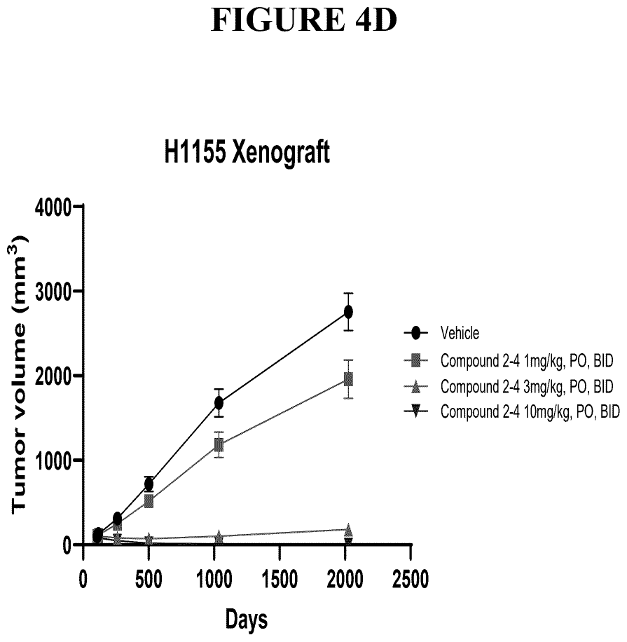

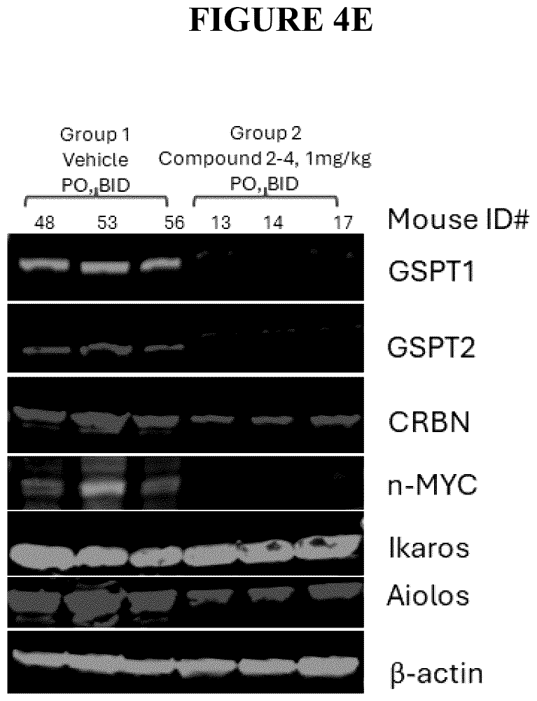

The LK2 (c-Myc overexpressing, Lung Squamous Cell Carcinoma) tumor cells were cultured in RPMI-1640 medium supplemented with 10% heat inactivated fetal bovine serum, 100 U/mL penicillin, and 100 μg/mL streptomycin at 37° C. in an atmosphere of 5% CO₂ in air. The tumor cells were routinely sub-cultured 2 to 3 times weekly. The cells growing in an exponential growth phase were harvested and counted for tumor inoculation. Each NOD/SCID female mouse was inoculated subcutaneously at the right flank with the LK2 tumor cells (5e6 per mouse) in 0.1 mL RPMI-1640 medium with 50% Matrigel for tumor development. A total of 40 mice were randomized into treatment groups, with 10 mice per group (n=10/group) assigned to either vehicle or compound administration. Test Compound 1-11 were dissolved using 5% NMP+50% PEG400+45% (20% HP-β-CD) at appropriate concentrations (0.5, 1 and 2 mg/mL for administering 5, 10 and 20 mg/kg). Desired amount of test compounds determined based on final concentrations were weighed and transferred to a glass vial, then appropriate volume of NMP and PEG400 were added, with vertexing after each addition and finally appropriate volume of HP-β-CD was added and vortexed or sonicated, as needed. Test compounds were prepared fresh daily and orally administered twice daily (BID dosing) for 28 days. Tumor volume was measured twice weekly in two dimensions using a caliper, and the volume was expressed in mm³ using the formula: V=0.5 a×b2, where a and b are the long and short diameters of the tumor, respectively. A two-way ANOVA was performed to compare body weight and tumor volume. All data was analyzed using GraphPad Prism 5 with p<0.05 considered to be statistically significant. In this study, the therapeutic efficacy of test compound 1-11 on tumor size in LK2 subcutaneous model in NOD/SCID mice in different groups at different time points are shown in FIG. 4A. All groups showed significant tumor inhibition. Treatment with Compound 1-11, 20 mg/kg, 10 mk/kg and 5 mg/kg all showed significant inhibition on LK2 tumor growth in dose dependent manner, the TGIs (Tumor Growth Inhibition) were 71.57%, 47.08% and 30.29% respectively (FIG. 4A).

Test Compound 2-4 dissolved using 5% NMP+50% PEG400+45% (20% HP-β-CD) were administered at 3, 10 and 30 mg/kg. Test compounds were prepared fresh daily and orally administered twice daily (BID dosing) for 28 days. Tumor volume was measured twice weekly in two dimensions using a caliper, and the volume was expressed in mm³ using the formula: V=0.5 a×b2, where a and b are the long and short diameters of the tumor, respectively. Effects on tumor size in different groups at different time points are shown in FIG. 4B. All treatment groups showed significant inhibition on tumor growth in dose dependent manner, the TGIs (Tumor Growth Inhibition) were 76.29%, 97.43% and 100.00% respectively. Treatment with Compound 2-4 30 mk/kg showed the most significant inhibition on LK2 tumor growth, the TGIs was 100.00% (FIG. 4B).

The HUH7 (N-Myc overexpressing, Hepatocellular carcinoma) tumor cells were cultured in DMEM medium supplemented with 10% heat inactivated fetal bovine serum, 100 U/mL penicillin and 100 μg/mL streptomycin at 37° C. in an atmosphere of 5% CO₂ in air. The tumor cells were routinely sub-cultured 2 to 3 times weekly. The cells growing in an exponential growth phase were harvested and counted for tumor inoculation. Each NOD/SCID female mouse was inoculated subcutaneously at the right flank with the HUH7 tumor cells (1e7 per mouse) in 0.1 mL DMEM medium with 50% Matrigel for tumor development. A total of 40 mice were randomized for vehicle and compound treatment, with n=10 mice/group. Test Compound 2-4 were dissolved using 5% NMP+50% PEG400+45% (20% HP-β-CD) at appropriate concentrations (0.1, 0.3 and 1 mg/mL for administering 1, 3 and 10 mg/kg). Desired amount of the test compounds determined based on final concentrations were weighed and transferred to a glass vial, then appropriate volume of NMP and PEG400 were added, with vertexing after each addition and finally appropriate volume of HP-β-CD was added and vortexed or sonicated, as needed. Test compounds were prepared fresh daily and orally administered twice daily (BID dosing) for 28 days. Tumor volume was measured twice weekly in two dimensions using a caliper, and the volume was expressed in mm³ using the formula: V=0.5 a×b2, where a and b are the long and short diameters of the tumor, respectively. A two-way ANOVA was performed to compare body weight and tumor volume. All data was analyzed using GraphPad Prism 5 with p<0.05 considered to be statistically significant. Effects on tumor size in different groups at different time points are shown in FIG. 4C. All treatment groups showed significant inhibition on HUH7 tumor growth, the TGIs were 78.86%, 71.35% and 100.00% respectively. Treatment with Compound 2-4 10 mg/kg showed the most significant inhibition on tumor growth, the TGIs was 100.00% (FIG. 4C).

The H1155 (N-Myc overexpressing, Lung Squamous Cell Carcinoma) tumor cells were cultured in DMEM medium supplemented with 10% heat inactivated fetal bovine serum, 100 U/mL penicillin and 100 μg/mL streptomycin at 37° C. in an atmosphere of 5% CO₂ in air. The tumor cells were routinely sub-cultured 2 to 3 times weekly. The cells growing in an exponential growth phase were harvested and counted for tumor inoculation. Each NOD/SCID female mouse was inoculated subcutaneously at the right flank with the H1155 tumor cells (2e6 per mouse) in 0.1 mL DMEM medium with 50% Matrigel for tumor development. A total of 40 mice were randomized for vehicle and compound treatment, with n=10 mice/group. Test Compound 2-4 were dissolved using 5% NMP+50% PEG400+45% (20% HP-β-CD) at appropriate concentrations (0.1, 0.3, and 1 mg/mL for administering 1, 3 and 10 mg/kg). Desired amount of test compounds determined based on final concentrations were weighed and transferred to a glass vial, then appropriate volume of NMP and PEG400 were added, with vertexing after each addition and finally appropriate volume of HP-β-CD was added and vortexed or sonicated, as needed. Test compounds were prepared fresh daily and orally administered twice daily (BID dosing) for 28 days. Tumor volume was measured twice weekly in two dimensions using a caliper, and the volume was expressed in mm³ using the formula: V=0.5 a×b2, where a and b are the long and short diameters of the tumor, respectively. A two-way ANOVA was performed to compare body weight and tumor volume. All data was analyzed using GraphPad Prism 5 with p<0.05 considered to be statistically significant. Effects on tumor size in different groups at different time points are shown in FIG. 4D. Effects on tumor size in different groups at different time points are shown in FIG. 4D. All treatment groups showed significant inhibition on H1155 tumor growth, with Compound 2-4, 1 mg/kg, 3 mg/kg, and 10 mg/kg all showed significant inhibition on tumor growth in dose dependent manner, the TGIs (Tumor Growth Inhibition) were 20.69%, 92.15% and 99.91% respectively (FIG. 4D).

Protein degradation with Compound 2-4 from in vivo efficacy with N-Myc overexpressing, HUH7 (Hepatocellular carcinoma), shown in FIG. 4C, was determined by western blot. Snap frozen tumors harvested on day 29, following 24 hours after completing 1 mg/kg with Compound 2-4 or vehicle (on day 28), were used for preparing protein lysates. Thin slices cut using sterile blade were transferred into 2 mL microfuge tubes prefilled with 3.0 mm zirconium beads (#D1032-30, Lot #3100, Millipore) and added 500 μL of 1× Cell Lysis Buffer (#98035, Lot #83, Cell Signaling Technology) consisting of protease/phosphatase inhibitor cocktail (#5872S, Lot #22, Cell Signaling Technology). Tubes were then transferred to BeadBug 6 homogenizer (Benchmark) for tissue disruption, pulsed for 10 seconds at maximum speed, with 10 seconds intervals for 2 minutes. Samples were then centrifuged at 14,000 rpm for 15 minutes at 4° C., using Labnet Prism R, refrigerated microcentrifuge. Supernatants were collected, and protein estimation and western blots were performed as described before. Results shown in FIG. 4E for compound 2-4, at a dose of 1 mg/kg twice daily, resulted in potent degradation of GSPT1 and GSPT2, and complete reduction in N-Myc protein levels in all three mice (#13, #14 & #17), compared to vehicle treated mice (#48, #53 & #56). With slight reduction in CRBN, the levels of CRBN neo-substrates Ikaros and Aiolos remained unchanged, and β-actin was used as loading control (FIG. 4E).

The compounds of Formulas (1-11) and (2-4) exhibited potent pharmacological activities. As used herein, the term “potency of growth-inhibitory activity” refers to the concentration (in nM) required to inhibit cell growth by 50% (IC₅₀). The results are summarized in Table 26 below, in which the degree of growth inhibition is categorized as follows: an IC₅₀ value of less than 11 nM is designated as “A”; an IC₅₀ value between 11 nM and 50 nM as “B”; an IC₅₀ value between 51 nM and 500 nM as “C”; an IC₅₀ value greater than 500 nM as “D”; and “ND” denotes that the IC₅₀ value was not determined.

Table 26 illustrates representative results of cell growth inhibition by CRBN modulators, exemplified by compounds of Formula (1-1). Across multiple cancer cell line panels, compounds 1-11 and 2-4 demonstrated strong inhibitory potency. In the colorectal adenocarcinoma panel, compound 1-11 exhibited IC₅₀ values of <100 nM for all tested cell lines, while compound 2-4 also showed IC₅₀ values of <100 nM except in NCIH747 and HCT116. In the hepatocellular carcinoma panel, compound 1-11 demonstrated IC₅₀ values of <100 nM for all cell lines except SNU449, and compound 2-4 exhibited IC₅₀ values of <100 nM except in SK-HEP-1 cells. In the non-small-cell lung cancer (NSCLC) panel consisting of lung squamous cell carcinoma lines, most of the tested cell lines showed IC₅₀ values of <50 nM for compound 1-11, and similarly potent activity for compound 2-4, except in HCC15 and HCC95. Overall, both compounds 1-11 and 2-4 demonstrated strong potency across multiple cancer types, reducing cell viability by more than 50% at concentrations below 100 nM within 72 hours.

TABLE 26
Cell proliferation IC₅₀ values

| Cell lines | Cancer | 1-11 | 2-4 |
|---|---|---|---|
| SW948 | Colorectal adenocarcinoma | A | B |
| COLO201 | Colorectal adenocarcinoma | A | B |
| SW480 | Colorectal adenocarcinoma | B | B |
| NCIH747 | Colorectal adenocarcinoma | A | D |
| HCT116 | Colorectal adenocarcinoma | B | D |
| HCT15 | Colorectal adenocarcinoma | C | B |
| HepG2 | Hepatobastoma | A | D |
| HepG2/C3A | Hepatobastoma | A | ND |
| HUH7 | Hepatocellular carcinoma | A | A |
| Hep3B | Hepatocellular carcinoma | ND | A |
| SNU449 | Hepatocellular carcinoma | B | B |
| SK-HEP1 | Hepatocellular carcinoma | B | C |
| SNU387 | Hepatocellular carcinoma | B | ND |
| SNU475 | Hepatocellular carcinoma | C | ND |
| A549 | Lung Adenocarcinoma | B | D |
| VERO | Monkey Kidney Epithelial | ND | C |
| | cells | | |
| LK2 | Non-Small Cell Lung Cancer | A | B |
| HCC95 | Non-Small Cell Lung Cancer | D | D |
| H1155 | Non-Small Cell Lung Cancer | A | A |
| H520 | Non-Small Cell Lung Cancer | A | B |
| NCIH2009 | Non-Small Cell Lung Cancer | A | B |
| HCC15 | Non-Small Cell Lung Cancer | B | D |
| NCIH1299 | Non-Small Cell Lung Cancer | A | ND |
| NCIH441 | Non-Small Cell Lung Cancer | A | ND |
| NCIH460 | Non-Small Cell Lung Cancer | B | ND |
| NCIH1975 | Non-Small Cell Lung Cancer | D | ND |
| SW900 | Non-Small Cell Lung Cancer | D | ND |
| NCIH1650 | Non-Small Cell Lung Cancer | B | ND |

Those skilled in the art will recognize that numerous modifications can be made to the specific implementations described above. The implementations should not be limited to the particular limitations described. Other implementations may be possible.

It is intended that the scope of the present compounds, compositions, and methods be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A compound represented by Formula (I), a pharmaceutically acceptable salt, diastereomer, enantiomer, racemate, hydrate, solvate, or prodrug thereof: 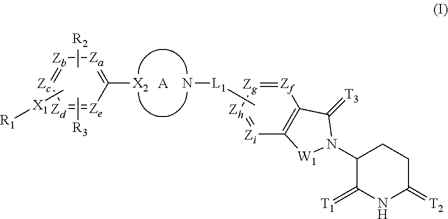
wherein:
L₁ is 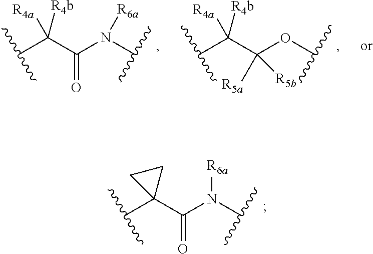
X₁ is a covalent bond, —O—, —S—, CHR₆ₐ, CR₆ₐR_{6b}, or NR₆ₐ;
X₂ is N, C, or CH;
Zₐ, Z_{b}, Z_{c}, Z_{d}, and Zₑ each are independently N or CR₅;
Z_{f}, Z_{g}, Zₕ, and Zᵢ each are independently N or CR₅;
T₁, T₂, and T₃ each are independently oxygen or sulfur;
W₁ is CH₂ or C(O); 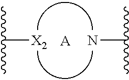
is 4-12 membered heterocyclic, 5-13 membered fused heterocycle, 5-13 membered bridged heterocycle, or 5-13 membered spiro heterocycle wherein 4-12 membered heterocyclic, 5-13 membered fused heterocycle, 5-13 membered bridged heterocycle, or 5-13 membered spiro heterocycle, which is optionally substituted with one or more groups independently selected from the group consisting of —C₁₋₃alkyl, —C₁₋₃ haloalkyl, CD₃, -oxo, —C₀₋₃ NR₆ₐR_{6b}, halogen, and —C₀₋₃ OH;
R₁ is hydrogen, 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, or 3-11 membered heterocycle, wherein 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, or 3-11 membered heterocycle, which is optionally substituted with one or more groups independently selected from the group consisting of halogen, —CN, —CD₃, —NO₂, —C(O)R₆ₐ, —NR₆ₐR_{6b}, —S(O)₂NR₆ₐR_{6b}, —NR₆ₐS(O)₂R_{6b}, C₁₋₆ haloalkoxy, —COOH, —CHO, furan, benzyloxy, —NR₆ₐC(O)R_{6b}, NR₆ₐC(O)NHR_{6b}, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, and C₁₋₁₅ heteroalkyl, wherein C₁₋₆ haloalkoxy, —COOH, —CHO, furan, benzyloxy, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, or C₁₋₁₅ heteroalkyl is optionally substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙO(CH₂)ₘ—NR₆ₐR_{6b}, —((CH₂)ₙO)ₘ—(CH₂)ₒ—NR₆ₐR_{6b}, —NR₆ₐC(O)(CH₂)ₙOH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR_{6b}, and —NR₆ₐ(CH₂)ₙNR_{6b}R_{6c};
R₂ and R₃ each are independently hydrogen, halogen, —CD₃, —CN, —NO₂, —S(O)₂NR₆ₐR_{6b}, —NR₆ₐS(O)₂R_{6b}, —C₁₋₆ haloalkoxy, —COOH, —CHO, —C(O)R₆ₐ, —NR₆ₐC(O)R_{6b}, —(CO)NR₆ₐR_{6b}, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl, wherein —C₁₋₆ haloalkoxy, —CHO, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl is optionally substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙ—O—(CH₂)ₘ—NR₆ₐR_{6b}, —((CH₂)ₙO)ₘ—(CH₂)ₒ—NR₆ₐR_{6b}, —NR₆ₐC(O)(CH₂)ₙCH₂OH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR₆ₐ, and —NR₆ₐ(CH₂)ₙNR_{6b}R_{6c};
R₄ₐ, R_{4b}, R₅ₐ, and R_{5b} each are independently hydrogen, halogen, —OH, —OCH₃, —CH₂CH₃, —CH₃, —CD₃, CN, —CF₃, —CHF₂, —CMeF₂, —CH₂CF₃, —CH₂CHF₂, optionally substituted with C₃₋₅ cycloalkyl, C₁₋₆ alkyl, C₁₋₆ alkylOH, C₁₋₆ alkylNH₂, or C₁₋₆ alkylNHR₆ₐ;
R₅ is hydrogen, halogen, —OH, —NH₂, —NHCH₃, C₁₋₃ alkyl, or C₁₋₃ alkoxy;
R₆ₐ, R_{6b}, and R_{6c} each are independently hydrogen, —CD₃, —S(O)₂R₇ₐ, aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl, wherein aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl is optionally substituted with one or more groups independently selected from the group consisting of —C₀₋₆ alkylNR₇ₐR_{7b}, —NR₇ₐC(O)(CH₂)ₙOH, —NR₇ₐC(O)(CH₂)ₙ—O—(CH₂)ₘ—OH, —NR₇ₐC(O)C₁₋₆ alkylNR_{7b}R_{7c}, —NR₇ₐC(O)C₁₋₆ alkylOR_{7b}, —NR₇ₐC(O)C₁₋₆ alkylSR_{7b}, —C(O)R₇ₐ, —C(O)C₀₋₆ alkylOR₇ₐ, —C(O)C₀₋₆ alkylNR₇ₐR_{7b}, —C(O)NR₇ₐR_{7b}, —C(O)NR₇ₐC₁₋₆ alkylNR_{7b}R_{7c}, —C(O)OC₁₋₆ alkylNR₇ₐR_{7b}, —C(O)OC₁₋₆ alkylOR₇ₐ, —(O(CH₂)ₙ)ₘOR₇ₐ, —O(CH₂)ₙC₁₋₆ cycloalkylNR₇ₐR_{7b}, —(CH₂)ₙO(CH₂)ₘ—NR₇ₐR_{7b}, —((CH₂)ₙO)ₘ(CH₂)ₒ—NR₇ₐR_{7b}, —NR₇ₐC(O)(CH₂), OH, —(O(CH₂)ₙ)ₘSR₇ₐ, —(O(CH₂)ₙ)ₘNR₇ₐR_{7b}, —(NR₇ₐ(CH₂)ₙ)ₘOR_{7b}, —(NR₇ₐ(CH₂)ₙ)ₘSR_{7b}, and —NR₇ₐ(CH₂)ₙNR_{7b}R_{7c};
R₇ₐ, R_{7b}, and R_{7c} each are independently hydrogen, halogen, —C₀₋₃ CN, —C₀₋₃ C(O)NH₂, —C₀₋₃ C(O)NHCH₃, —C₀₋₃ C(O)N(CH₃)₂, —CD₃, benzyl, —C₃₋₆ cycloalkyl, 4-7 membered heterocyclic, C₁₋₆ alkyl, —C₀₋₆ alkylOH, —C₀₋₆ alkylOCH3, —C₀₋₆ alkylNH₂, —C₀₋₆ alkylNHCH3, —C₀₋₆ haloalkylNHCH₃, —C₀₋₆ alkylC(O)OH, —C₀₋₆ alkylC(O)OCH₃, —C₀₋₆ alkylSH, —C₁₋₆ alkoxy, —C₂₋₆ alkenyl, —C₂₋₆ alkynyl, —C₁₋₆ haloalkyl, —C₁₋₁₀ heteroalkyl, —C(O)C₁₋₆ OH, —C(O)C₁₋₆ NH₂, or —C(O)C₁₋₃ NHC₁₋₃ alkyl; and
n, m, and o each are independently an integer of 1, 2, 3, 4, or 5.

2. The compound of claim 1, wherein Ring A is azetidine, pyrrolidine, piperidine, 1,2,3,6-tetrahydropyridine, piperazine, 3,6-diazabicyclo[3.1.1]heptane, piperidin-2-one, 1,4-diazepane, piperazin-2-one, 3-azaspiro[5.5]undecane, 3,9-diazaspiro[5.5]undecane, 2,6-diazaspiro[3.4]octane, or 2,6-diazaspiro[3.3]heptane, each optionally substituted with one or two substituents independently selected from the group consisting of cyclopropyl, -Me, Oxo, and —CF₃.

3. The compound of claim 1, wherein R₁ is hydrogen, 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, or 3-11 membered heterocycle.

4. The compound of claim 3, wherein 2,3-dihydro-1H-indene, 6,7-dihydro-5H-pyrrolo[3,4-b]pyridine, 5,6,7,8-tetrahydro-1,6-naphthyridine, isoindoline, 6,7-dihydro-5H-pyrrolo[3,4-d]pyrimidine, C₃₋₈ cycloalkyl, 5-10 membered aryl, 5-7 membered heteroaryl, 7-11 membered spiro ring, or 3-11 membered heterocycle is substituted with one or more groups independently selected from the group consisting of halogen, —CN, CD₃, —NO₂, —C(O)R₆ₐ, —NR₆ₐR_{6b}, —S(O)₂NR₆ₐR_{6b}, —NR₆ₐS(O)₂R_{6b}, C₁₋₆ haloalkoxy, —COOH, —CHO, furan, benzyloxy, —NR₆ₐC(O)R_{6b}, NR₆ₐC(O)NHR_{6b}, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, or C₁₋₁₅ heteroalkyl, wherein C₁₋₆ haloalkoxy, —COOH, —CHO, furan, benzyloxy, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, and C₁₋₁₅ heteroalkyl.

5. The compound of claim 4, wherein C₁₋₆ haloalkoxy, —COOH, —CHO, furan, benzyloxy, C₃₋₇ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₂₋₆ haloalkenyl, C₂₋₆ haloalkynyl, C₁₋₆ haloalkyl, or C₁₋₁₅ heteroalkyl is substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙO(CH₂)ₘ—NR₆ₐR_{6b}, —((CH₂)ₙO)ₘ—(CH₂)ₒ—NR₆ₐR_{6b}, —NR₆ₐC(O)(CH₂)ₙOH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR_{6b}, and —NR₆ₐ(CH₂)ₙNR_{6b}R_{6c}.

6. The compound of claim 1, wherein R₂ and R₃ each are independently hydrogen, halogen, —CD₃, —CN, —NO₂, —S(O)₂NR₆ₐR_{6b}, —NR₆ₐS(O)₂R_{6b}, —C₁₋₆ haloalkoxy, —COOH, —CHO, —C(O)R₆ₐ, —NR₆ₐC(O)R_{6b}, —(CO)NR₆ₐR_{6b}, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl.

7. The compound of claim 6, wherein C₁₋₆ haloalkoxy, —CHO, C₃₋₈ cycloalkyl, 4-8 membered heterocyclic, C₁₋₆ alkyl, C₀₋₆ alkylOH, C₀₋₆ alkylNH₂, C₀₋₆ alkylC(O)OH, C₀₋₆ alkylSH, C₁₋₆ alkoxy, C₂₋₆ alkenyl, C₂₋₆ alkynyl, C₁₋₆ haloalkyl, or C₁₋₁₀ heteroalkyl is substituted with one or more groups independently selected from the group consisting of —C₀₋₆ alkylNR₆ₐR_{6b}, —NR₆ₐC(O)(CH₂)ₙOH, —NR₆ₐC(O)(CH₂)ₙ—O—(CH₂)ₘ—OH, —NR₆ₐC(O)C₁₋₆ alkylNR_{6b}R_{6c}, —NR₆ₐC(O)C₁₋₆ alkylOR_{6b}, —NR₆ₐC(O)C₁₋₆ alkylSR_{6b}, —C(O)R₆ₐ, —C(O)C₀₋₆ alkylOR₆ₐ, —C(O)C₀₋₆ alkylNR₆ₐR_{6b}, —C(O)NR₆ₐR_{6b}, —C(O)NR₆ₐC₁₋₆ alkylNR_{6b}R_{6c}, —C(O)OC₁₋₆ alkylNR₆ₐR_{6b}, —C(O)OC₁₋₆ alkylOR₆ₐ, —(O(CH₂)ₙ)ₘOR₆ₐ, —O(CH₂)ₙC₁₋₆ cycloalkylNR₆ₐR_{6b}, —(CH₂)ₙ—O—(CH₂)ₘ—NR₆ₐR_{6b}, —((CH₂)ₙO)ₘ—(CH₂)ₒ—NR₆ₐR_{6b}, —NR₆ₐC(O)(CH₂)ₙCH₂OH, —(O(CH₂)ₙ)ₘSR₆ₐ, —(O(CH₂)ₙ)ₘNR₆ₐR_{6b}, —(NR₆ₐ(CH₂)ₙ)ₘOR_{6b}, —(NR₆ₐ(CH₂)ₙ)ₘSR₆ₐ, and —NR₆ₐ(CH₂)ₙNR_{6b}R_{6c}.

8. The compound of claim 1, wherein R₄ₐ, R_{4b}, R₅ₐ, and R_{5b} in L each are independently hydrogen, halogen, —OH, —OCH₃, —CH₂CH₃, —CH₃, —CD₃, CN, —CF₃, —CHF₂, —CMeF₂, —CH₂CF₃, or —CH₂CHF₂, each optionally substituted with one or more groups independently selected from the group consisting of C₃₋₅ cycloalkyl, C₁₋₆ alkyl, C₁₋₆ alkylOH, C₁₋₆ alkylNH₂, and C₁₋₆ alkylNHR₆ₐ.

9. The compound of claim 1, wherein R₅ is hydrogen, halogen, —OH, —NH₂, —NHCH₃, C₁₋₃ alkyl, or C₁₋₃ alkoxy.

10. The compound of claim 1, wherein R₆ₐ, R_{6b}, and R_{6c} each are independently hydrogen, —CD₃, —S(O)₂R₇ₐ, aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl, which is optionally substituted with one or more groups independently selected from the group consisting of —NR₇ₐR_{7b}, —NR₇ₐC(O)(CH₂)ₙOH, —NR₇ₐC(O)(CH₂)ₙ—O—(CH₂)ₙ—OH, —NR₇ₐC(O)—C₁₋₆ alkylNH₂, -alkylNHR₇ₐ, —NR₇ₐC(O)—C₁₋₆ alkylOH, —NR₇ₐC(O)—C₁₋₆-alkylSH, —C(O)O—C₁₋₆ alkylNH₂, -alkylNHR₇ₐ —C(O)O—C₁₋₆-alkylOH, —(CH₂)ₙ—O—(CH₂)ₙ—NR₇ₐR_{7b}, —NR₇ₐC(O)(CH₂)ₙCH₂R_{7b}OH, —C(O)NR₇ₐ(CH)ₙNR₇ₐR_{7b}, —(O(CH₂)ₙ)ₘOH, —(O(CH₂)ₙ)ₘSH, —(O(CH₂)ₙ)ₘNR₇ₐR_{7b}, —(NR₇ₐ(CH₂)ₙ)ₘOH, —(NR₇ₐ(CH₂)ₙ)ₘSH, and —(NR₇ₐ(CH₂)ₙ)ₘNR₇ₐR_{7b}.

11. The compound of claim 10, wherein aryl, 5-7-membered heteroaryl, —C₃₋₆ cycloalkyl, 4-7 membered heterocyclic, —C₁₋₆ alkyl, —C₀₋₆ alkylOH, —C₀₋₆ alkylNH₂, —C₀₋₆ alkylCN, —C₀₋₆ alkylC(O)OH, —C₀₋₆ alkylSH, —C₁₋₆ alkoxy, —C₂₋₆ alkenyl, —C₂₋₆ alkynyl, —C₁₋₁₀ haloalkyl, or C₁₋₁₅ heteroalkyl is substituted with one or more groups independently selected from the group consisting of —C₀₋₆ alkylNR₇ₐR_{7b}, —NR₇ₐC(O)(CH₂)ₙOH, —NR₇ₐC(O)(CH₂)ₙ—O—(CH₂)ₘ—OH, —NR₇ₐC(O)C₁₋₆ alkylNR_{7b}R_{7c}, —NR₇ₐC(O)C₁₋₆ alkylOR_{7b}, —NR₇ₐC(O)C₁₋₆ alkylSR_{7b}, —C(O)R₇ₐ, —C(O)C₀₋₆ alkylOR₇ₐ, —C(O)C₀₋₆ alkylNR₇ₐR_{7b}, —C(O)NR₇ₐR_{7b}, —C(O)NR₇ₐC₁₋₆ alkylNR_{7b}R_{7c}, —C(O)OC₁₋₆ alkylNR₇ₐR_{7b}, —C(O)OC₁₋₆ alkylOR₇ₐ, —(O(CH₂)ₙ)ₘOR₇ₐ, —O(CH₂)ₙC₁₋₆ cycloalkylNR₇ₐR_{7b}, —(CH₂)ₙO(CH₂)ₘ—NR₇ₐR_{7b}, —((CH₂)ₙO)ₘ(CH₂)ₒ—NR₇ₐR_{7b}, —NR₇ₐC(O)(CH₂)ₙOH, —(O(CH₂)ₙ)ₘSR₇ₐ, —(O(CH₂)ₙ)ₘNR₇ₐR_{7b}, —(NR₇ₐ(CH₂)ₙ)ₘOR_{7b}, —(NR₇ₐ(CH₂)ₙ)ₘSR_{7b}, and —NR₇ₐ(CH₂)ₙNR_{7b}R_{7c}.

12. The compound of claim 1, wherein R₇ₐ, R_{7b}, and R_{7c} each are independently hydrogen, halogen, —C₀₋₃ CN, —C₀₋₃ C(O)NH₂, —C₀₋₃ C(O)NHCH₃, —C₀₋₃ C(O)N(CH₃)₂, —CD₃, benzyl, —C₃₋₆ cycloalkyl, 4-7 membered heterocyclic, C₁₋₆ alkyl, —C₀₋₆ alkylOH, —C₀₋₆ alkylOCH3, —C₀₋₆ alkylNH₂, —C₀₋₆ alkylNHCH3, —C₀₋₆ haloalkylNHCH₃, —C₀₋₆ alkylC(O)OH, —C₀₋₆ alkylC(O)OCH₃, —C₀₋₆ alkylSH, —C₁₋₆ alkoxy, —C₂₋₆ alkenyl, —C₂₋₆ alkynyl, —C₁₋₆ haloalkyl, —C₁₋₁₀ heteroalkyl, —C(O)C₁₋₆ OH, —C(O)C₁₋₆ NH₂, or —C(O)C₁₋₃ NHC₁₋₃ alkyl.

13. The compound of claim 1, wherein n, m, and o are independently an integer of 1, 2, or 3.

14. The compound of claim 1, which is selected from the group consisting of:
N-{2-[(S)-2,6-dioxo-3-piperidyl]-1-oxo-5-isoindolinyl}[3-(p-bromophenyl)-1-azetidinyl]acetamide
N-{2-[(S)-2,6-dioxo-3-piperidyl]-1-oxo-5-isoindolinyl}[4-(o-fluorophenyl)-1-piperazinyl]acetamide
N-{2-[(S)-2,6-dioxo-3-piperidyl]-1-oxo-5-isoindolinyl}[4-(o-methoxyphenyl)-1-piperazinyl]acetamide
N-{2-[(S)-2,6-dioxo-3-piperidyl]-1-oxo-5-isoindolinyl}{4-[2-methoxy-4-(2-pyridylamino)phenyl]-1-piperazinyl}acetamide
N-{2-[(S)-2,6-dioxo-3-piperidyl]-1-oxo-5-isoindolinyl}{4-[4-(2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(2-pyridylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}propionamide
N-{2-[(S)-2,6-dioxo-3-piperidyl]-1-oxo-5-isoindolinyl}2-{4-[2-methoxy-4-(2-pyridylamino)phenyl]-1-piperazinyl}propionamide
N-{2-[(S)-2,6-dioxo-3-piperidyl]-1-oxo-5-isoindolinyl}2-{4-[4-(2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[4-(6-chloro-4-pyrimidinylamino)-2-fluorophenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(6-chloro-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(2-pyrimidinylamino)phenyl]-1-piperazinyl}acetamide
N-{2-[(S)-2,6-dioxo-3-piperidyl]-1-oxo-5-isoindolinyl}{4-[4-amino-2-(trifluoromethyl)phenyl]-1-piperazinyl}acetamide
N-{2-[(S)-2,6-dioxo-3-piperidyl]-1-oxo-5-isoindolinyl}{4-[4-(4-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[p-(2-pyridylamino)phenyl]-1-azetidinyl}acetamide
N-{2-[(S)-2,6-dioxo-3-piperidyl]-1-oxo-5-isoindolinyl}2-{3-[p-(2-pyridylamino)phenyl]-1-azetidinyl}propionamide
N-{2-[(S)-2,6-dioxo-3-piperidyl]-1-oxo-5-isoindolinyl}{4-[2-methoxy-4-(2-pyrimidinylamino)phenyl]-1-piperazinyl}acetamide
N-{2-[(S)-2,6-dioxo-3-piperidyl]-1-oxo-5-isoindolinyl}{4-[2-chloro-4-(4-pyrimidinylamino)phenyl]-1-piperazinyl}acetamide
N-{2-[(S)-2,6-dioxo-3-piperidyl]-1-oxo-5-isoindolinyl}{4-[3-methoxy-4-(2-pyrimidinylamino)phenyl]-1-piperazinyl}acetamide
N-{2-[(S)-2,6-dioxo-3-piperidyl]-1-oxo-5-isoindolinyl}{4-[3-methoxy-4-(2-pyridylamino)phenyl]-1-piperazinyl}acetamide
N-{2-[(S)-2,6-dioxo-3-piperidyl]-1-oxo-5-isoindolinyl}{4-[2-fluoro-4-(2-pyridylamino)phenyl]-1-piperazinyl}acetamide
N-{2-[(S)-2,6-dioxo-3-piperidyl]-1-oxo-5-isoindolinyl}{4-[2-fluoro-4-(2-pyrimidinylamino)phenyl]-1-piperazinyl}acetamide
N-{2-[(S)-2,6-dioxo-3-piperidyl]-1-oxo-5-isoindolinyl}{4-[2-fluoro-4-(4-pyrimidinylamino)phenyl]-1-piperazinyl}acetamide
N-{2-[(S)-2,6-dioxo-3-piperidyl]-1-oxo-5-isoindolinyl}{4-[p-(2-pyridylamino)phenyl]-1-piperazinyl}acetamide
N-{2-[(S)-2,6-dioxo-3-piperidyl]-1-oxo-5-isoindolinyl}{4-[p-(2-pyrimidinylamino)phenyl]-1-piperazinyl}acetamide
N-{2-[(S)-2,6-dioxo-3-piperidyl]-1-oxo-5-isoindolinyl}{4-[4-(2-pyridylamino)-2-tolyl]-1-piperazinyl}acetamide
N-{2-[(S)-2,6-dioxo-3-piperidyl]-1-oxo-5-isoindolinyl}{4-[4-(2-pyrimidinylamino)-2-tolyl]-1-piperazinyl}acetamide
N-{2-[(S)-2,6-dioxo-3-piperidyl]-1-oxo-5-isoindolinyl}{4-[4-(6-chloro-4-pyrimidinylamino)-2-tolyl]-1-piperazinyl}acetamide
N-{2-[(S)-2,6-dioxo-3-piperidyl]-1-oxo-5-isoindolinyl}(S)-2-{4-[2-chloro-4-(2-pyridylamino)phenyl]-1-piperazinyl}propionamide
N-{2-[(S)-2,6-dioxo-3-piperidyl]-1-oxo-5-isoindolinyl}(R)-2-{4-[2-chloro-4-(2-pyridylamino)phenyl]-1-piperazinyl}propionamide
N-{2-[(S)-2,6-dioxo-3-piperidyl]-1-oxo-5-isoindolinyl}{4-[4-(4,6-dimethyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}2-{4-[2-chloro-4-(2-pyrimidinylamino)phenyl]-1-piperazinyl}propionamide
N-{2-[(S)-2,6-dioxo-3-piperidyl]-1-oxo-5-isoindolinyl}2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}2-{4-[4-(6-chloro-4-pyrimidinylamino)-2-tolyl]-1-piperazinyl}propionamide
N-{2-[(S)-2,6-dioxo-3-piperidyl]-1-oxo-5-isoindolinyl}{4-[2,6-difluoro-4-(2-pyrimidinylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}butyramide
N-{2-[(S)-2,6-dioxo-3-piperidyl]-1-oxo-5-isoindolinyl}{4-[2-chloro-4-(4,6-dimethyl-2-pyrimidinylamino)phenyl]-1-piperazinyl}acetamide
N-{2-[(S)-2,6-dioxo-3-piperidyl]-1-oxo-5-isoindolinyl}{4-[4-(4,6-dimethyl-2-pyrimidinylamino)-2-methoxyphenyl]-1-piperazinyl}acetamide
N-{2-[(S)-2,6-dioxo-3-piperidyl]-1-oxo-5-isoindolinyl}(R)-2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{4-[2-cyano-4-(4,6-dimethyl-2-pyrimidinylamino)phenyl]-1-piperazinyl}acetamide
N-{2-[(S)-2,6-dioxo-3-piperidyl]-1-oxo-5-isoindolinyl}{4-[4-(4-cyano-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(6-methyl-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}(R)-2-{4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{3-oxo-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{(R)-2-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-cyano-4-(2-pyrimidinylamino)phenyl]-1-piperazinyl}acetamide
N-{2-[(S)-2,6-dioxo-3-piperidyl]-1-oxo-5-isoindolinyl}(S)-2-{4-[2-cyano-4-(2-pyrimidinylamino)phenyl]-1-piperazinyl}propionamide
N-{2-[(S)-2,6-dioxo-3-piperidyl]-1-oxo-5-isoindolinyl}{(S)-2-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{(R)-3-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methoxy-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[4-(4-methoxy-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{(S)-3-methyl-4-[4-(2-pyrimidinylamino)-2-(trifluoromethyl)phenyl]-1-piperazinyl}acetamide
N-{2-[(S)-2,6-dioxo-3-piperidyl]-1-oxo-5-isoindolinyl}[4-(4-amino-2-chlorophenyl)-1-piperazinyl]acetamide
N-{2-[(S)-2,6-dioxo-3-piperidyl]-1-oxo-5-isoindolinyl}{4-[4-(4,6-dimethyl-2-pyrimidinylamino)-2-tolyl]-1-piperazinyl}acetamide
N-{2-[(S)-2,6-dioxo-3-piperidyl]-1-oxo-5-isoindolinyl}{4-[4-(2,6-dimethyl-4-pyrimidinylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[2-methoxy-4-(2-pyrimidinylamino)phenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyrimidinylamino)-2-methoxyphenyl]-1-piperazinyl}propionamide
N-{2-[(S)-2,6-dioxo-3-piperidyl]-1-oxo-5-isoindolinyl}{4-[4-(5-methyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methoxy-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1,4-diazepan-1-yl}acetamide
N-{2-[(S)-2,6-dioxo-3-piperidyl]-1-oxo-5-isoindolinyl}{4-[4-(methylamino)-2-(trifluoromethyl)phenyl]-1-piperazinyl}acetamide
N-{2-[(S)-2,6-dioxo-3-piperidyl]-1-oxo-5-isoindolinyl}{4-[4-(dimethylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-methyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(5-methyl-2-pyrimidinylamino)phenyl]-1-piperazinyl}acetamide
N-{2-[(S)-2,6-dioxo-3-piperidyl]-1-oxo-5-isoindolinyl}{4-[2-chloro-4-(methylamino)phenyl]-1-piperazinyl}acetamide
N-{2-[(S)-2,6-dioxo-3-piperidyl]-1-oxo-5-isoindolinyl}{4-[2-chloro-4-(dimethylamino)phenyl]-1-piperazinyl}acetamide
N-{2-[(S)-2,6-dioxo-3-piperidyl]-1-oxo-5-isoindolinyl}2-{4-[2-chloro-4-(2-pyridylamino)phenyl]-1-piperazinyl}-2-methylpropionamide
N-{2-[(S)-2,6-dioxo-3-piperidyl]-1-oxo-5-isoindolinyl}{3-[2-chloro-4-(2-pyridylamino)phenyl]-1-azetidinyl}acetamide
N-{2-[(S)-2,6-dioxo-3-piperidyl]-1-oxo-5-isoindolinyl}{3-[2-chloro-4-(4-methyl-2-pyridylamino)phenyl]-1-azetidinyl}acetamide
N-{2-[(S)-2,6-dioxo-3-piperidyl]-1-oxo-5-isoindolinyl}(S)-2-{4-[2-chloro-4-(4,6-dimethyl-2-pyrimidinylamino)phenyl]-1-piperazinyl}propionamide
N-{2-[(S)-2,6-dioxo-3-piperidyl]-1-oxo-5-isoindolinyl}{4-[2-chloro-4-(4-methyl-2-pyrimidinylamino)phenyl]-1-piperazinyl}acetamide
N-{2-[(S)-2,6-dioxo-3-piperidyl]-1-oxo-5-isoindolinyl}{4-[2-chloro-4-(2,6-dimethyl-4-pyrimidinylamino)phenyl]-1-piperazinyl}acetamide
N-{2-[(S)-2,6-dioxo-3-piperidyl]-1-oxo-5-isoindolinyl}{3-[4-(4-methyl-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[2-chloro-4-(2-pyrimidinylamino)phenyl]-1-azetidinyl}acetamide
N-{2-[(S)-2,6-dioxo-3-piperidyl]-1-oxo-5-isoindolinyl}{4-[2-chloro-4-(4-cyano-2-pyridylamino)phenyl]-1-piperazinyl}acetamide
N-{2-[(S)-2,6-dioxo-3-piperidyl]-1-oxo-5-isoindolinyl}{3-[4-(4,6-dimethyl-2-pyrimidinylamino)-2-(trifluoromethyl)phenyl]-3,6-diazabicyclo[3.1.1]hept-6-yl}acetamide
N-{2-[(S)-2,6-dioxo-3-piperidyl]-1-oxo-5-isoindolinyl}{(R)-3-methyl-4-[4-nitro-2-(trifluoromethyl)phenyl]-1-piperazinyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-4,7-diaza-7-spiro[2.5]octyl}acetamide
N-{2-[(S)-2,6-dioxo-3-piperidyl]-1-oxo-5-isoindolinyl}{4-[4-(4-methyl-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(4-methyl-2-pyridylamino)phenyl]-1-piperazinyl}acetamide
N-{2-[(S)-2,6-dioxo-3-piperidyl]-1-oxo-5-isoindolinyl}{3-[4-(2-pyrimidinylamino)-2-(trifluoromethyl)phenyl]-3,6-diazabicyclo[3.1.1]hept-6-yl}acetamide
N-{2-[(S)-2,6-dioxo-3-piperidyl]-1-oxo-5-isoindolinyl}{(S)-4-[4-amino-2-(trifluoromethyl)phenyl]-3-methyl-1-piperazinyl}acetamide
N-{2-[(S)-2,6-dioxo-3-piperidyl]-1-oxo-5-isoindolinyl}{(R)-4-[4-(4,6-dimethyl-2-pyrimidinylamino)-2-(trifluoromethyl)phenyl]-3-methyl-1-piperazinyl}acetamide
N-{2-[(S)-2,6-dioxo-3-piperidyl]-1-oxo-5-isoindolinyl}{(R)-4-[4-(4-cyano-2-pyridylamino)-2-(trifluoromethyl)phenyl]-3-methyl-1-piperazinyl}acetamide
N-{2-[(S)-2,6-dioxo-3-piperidyl]-1-oxo-5-isoindolinyl}(R)-2-{4-[2-chloro-4-(4,6-dimethyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-cyano-2-pyridylamino)phenyl]-1-piperazinyl}propionamide
N-{2-[(S)-2,6-dioxo-3-piperidyl]-1-oxo-5-isoindolinyl}(R)-2-{4-[4-(2,6-dimethyl-4-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(5-methyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(4-methyl-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{4-[4-(4-methyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(R)-2-{3-[4-(2-pyrimidinylamino)-2-(trifluoromethyl)phenyl]-1-azetidinyl}propionamide
N-{2-[(S)-2,6-dioxo-3-piperidyl]-1-oxo-5-isoindolinyl}(R)-2-{3-[2-chloro-4-(2-pyrimidinylamino)phenyl]-1-azetidinyl}propionamide
N-{2-[(S)-2,6-dioxo-3-piperidyl]-1-oxo-5-isoindolinyl}{4-[4-(N-methyl-N-2-pyrimidinylamino)-2-chlorophenyl]-1-piperazinyl}acetamide
N-{2-[(S)-2,6-dioxo-3-piperidyl]-1-oxo-5-isoindolinyl}[4-(4-bromo-2-chlorophenyl)-1-piperazinyl]acetamide
N-{2-[(S)-2,6-dioxo-3-piperidyl]-1-oxo-5-isoindolinyl}(R)-2-{(R)-4-[4-(4,6-dimethyl-2-pyrimidinylamino)-2-(trifluoromethyl)phenyl]-3-methyl-1-piperazinyl}propionamide
N-{2-[(S)-2,6-dioxo-3-piperidyl]-1-oxo-5-isoindolinyl}(R)-2-{4-[2-chloro-4-(5-methyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-methyl-2-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(2,6-dimethyl-4-pyrimidinylamino)phenyl]-1-piperazinyl}propionamide
N-{2-[(S)-2,6-dioxo-3-piperidyl]-1-oxo-5-isoindolinyl}(R)-2-{4-[2-chloro-4-(4-methyl-2-pyridylamino)phenyl]-1-piperazinyl}propionamide
N-{2-[(S)-2,6-dioxo-3-piperidyl]-1-oxo-5-isoindolinyl}{4-[2-chloro-4-(2-pyrimidinyloxy)phenyl]-1-piperazinyl}acetamide
N-{2-[(S)-2,6-dioxo-3-piperidyl]-1-oxo-5-isoindolinyl}[4-(2-chloro-4-cyanophenyl)-1-piperazinyl]acetamide
N-{2-[(S)-2,6-dioxo-3-piperidyl]-1-oxo-5-isoindolinyl}(R)-2-{(R)-3-methyl-4-[4-(2-pyrimidinylamino)-2-(trifluoromethyl)phenyl]-1-piperazinyl}propionamide
N-{2-[(S)-2,6-dioxo-3-piperidyl]-1-oxo-5-isoindolinyl}(R)-2-{(R)-3-methyl-4-[4-(2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[4-(4,6-dimethyl-2-pyridylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-chloro-2-pyridylamino)-2-(trifluoromethyl)phenyl]-1-piperazinyl}acetamide
N-{2-[(S)-2,6-dioxo-3-piperidyl]-1-oxo-5-isoindolinyl}(S)-2-{4-[4-(N-methyl-N-2-pyrimidinylamino)-2-chlorophenyl]-1-piperazinyl}propionamide
N-{2-[(S)-2,6-dioxo-3-piperidyl]-1-oxo-5-isoindolinyl}(S)-2-{4-[4-(4,6-dimethyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[4-(4-chloro-2-pyridylamino)-2-(trifluoromethyl)phenyl]-1-piperazinyl}propionamide
N-{2-[(S)-2,6-dioxo-3-piperidyl]-1-oxo-5-isoindolinyl}{4-[2-chloro-4-(6-methyl-4-pyrimidinylamino)phenyl]-1-piperazinyl}acetamide
N-{2-[(S)-2,6-dioxo-3-piperidyl]-1-oxo-5-isoindolinyl}[4-(2,4-dichlorophenyl)-1-piperazinyl]acetamide
N-{2-[(S)-2,6-dioxo-3-piperidyl]-1-oxo-5-isoindolinyl}{4-[2-chloro-4-(6-chloro-4-pyrimidinylamino)phenyl]-1-piperazinyl}acetamide
N-{2-[(S)-2,6-dioxo-3-piperidyl]-1-oxo-5-isoindolinyl}[4-(3,4-dichlorophenyl)-1-piperazinyl]acetamide
N-{2-[(S)-2,6-dioxo-3-piperidyl]-1-oxo-5-isoindolinyl}(R)-2-{4-[4-(2-pyrimidinylamino)-2-(trifluoromethyl)phenyl]-1-piperazinyl}butyramide
N-{2-[(S)-2,6-dioxo-3-piperidyl]-1-oxo-5-isoindolinyl}(S)-2-{3-[4-(2-pyridylamino)-2-(trifluoromethyl)phenyl]-1-azetidinyl}butyramide
N-{2-[(S)-2,6-dioxo-3-piperidyl]-1-oxo-5-isoindolinyl}(R)-2-{(R)-3-methyl-4-[4-(4-methyl-2-pyridylamino)-2-(trifluoromethyl)phenyl]-1-piperazinyl}propionamide
N-{2-[(S)-2,6-dioxo-3-piperidyl]-1-oxo-5-isoindolinyl}(S)-2-{4-[2-chloro-4-(4-methyl-2-pyridylamino)phenyl]-1-piperazinyl}butyramide
N-{2-[(S)-2,6-dioxo-3-piperidyl]-1-oxo-5-isoindolinyl}{3-[4-(N-methyl-N-4-pyrimidinylamino)-2-chlorophenyl]-1-azetidinyl}acetamide
N-{2-[(S)-2,6-dioxo-3-piperidyl]-1-oxo-5-isoindolinyl}(R)-2-{3-[4-(N-methyl-N-4-pyrimidinylamino)-2-chlorophenyl]-1-azetidinyl}propionamide
N-{2-[(S)-2,6-dioxo-3-piperidyl]-1-oxo-5-isoindolinyl}(R)-2-{4-[2-chloro-4-(4-methyl-2-pyridylamino)phenyl]-1-piperazinyl}butyramide
N-{2-[(S)-2,6-dioxo-3-piperidyl]-1-oxo-5-isoindolinyl}{3-[4-(N-methyl-N-2-pyrimidinylamino)-2-chlorophenyl]-1-azetidinyl}acetamide
N-{2-[(S)-2,6-dioxo-3-piperidyl]-1-oxo-5-isoindolinyl}(R)-2-{3-[4-(N-methyl-N-2-pyrimidinylamino)-2-chlorophenyl]-1-azetidinyl}propionamide
N-{2-[(S)-2,6-dioxo-3-piperidyl]-1-oxo-5-isoindolinyl}{4-[4-(1-azetidinyl)-2-(trifluoromethyl)phenyl]-1-piperazinyl}acetamide
N-{2-[(S)-2,6-dioxo-3-piperidyl]-1-oxo-5-isoindolinyl}{4-[2-(trifluoromethyl)-4-cumenyl]-1-piperazinyl}acetamide
N-{2-[(S)-2,6-dioxo-3-piperidyl]-1-oxo-5-isoindolinyl}{3-[4-(N-methyl-N-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}(R)-2-{3-[4-(N-methyl-N-2-pyrimidinylamino)-2-(trifluoromethyl)phenyl]-1-azetidinyl}propionamide
N-{2-[(S)-2,6-dioxo-3-piperidyl]-1-oxo-5-isoindolinyl}{4-[4-(4-isopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}(4-{4-[4-(1-pyrrolidinyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[2-chloro-4-(4-chloro-2-pyridylamino)phenyl]-1-piperazinyl}acetamide
N-{2-[(S)-2,6-dioxo-3-piperidyl]-1-oxo-5-isoindolinyl}{4-[4-(N-methyl-N-4-pyrimidinylamino)-2-chlorophenyl]-1-piperazinyl}acetamide
N-{2-[(S)-2,6-dioxo-3-piperidyl]-1-oxo-5-isoindolinyl}{4-[4-(5-isopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-(4-morpholino-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[4-(N-methyl-N-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}(4-{4-[4-methyl-6-(trifluoromethyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[4-(4-cyclopropyl-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{4-[4-chloro-2-(trifluoromethyl)phenyl]-1-piperazinyl}acetamide
N-{2-[(S)-2,6-dioxo-3-piperidyl]-1-oxo-5-isoindolinyl}(R)-2-{3-[4-(N-methyl-N-4-pyrimidinylamino)-2-(trifluoromethyl)phenyl]-1-azetidinyl}propionamide
N-{2-[(S)-2,6-dioxo-3-piperidyl]-1-oxo-5-isoindolinyl}(4-{4-[4-(tert-butyl)-2-pyrimidinylamino]-2-(trifluoromethyl)phenyl}-1-piperazinyl)acetamide
N-{2-[(S)-2,6-dioxo-3-piperidyl]-1-oxo-5-isoindolinyl}{4-[4-(N-methyl-N-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[2-chloro-4-(4-isopropyl-2-pyrimidinylamino)phenyl]-1-azetidinyl}acetamide
N-{2-[(S)-2,6-dioxo-3-piperidyl]-1-oxo-5-isoindolinyl}(3-{2-chloro-4-[4-(1-pyrrolidinyl)-2-pyrimidinylamino]phenyl}-1-azetidinyl)acetamide
N-{2-[(S)-2,6-dioxo-3-piperidyl]-1-oxo-5-isoindolinyl}{3-[2-chloro-4-(4-morpholino-2-pyrimidinylamino)phenyl]-1-azetidinyl}acetamide
N-{2-[(S)-2,6-dioxo-3-piperidyl]-1-oxo-5-isoindolinyl}(S)-2-{4-[4-(N-methyl-N-2-pyrimidinylamino)-2-(trifluoromethyl)phenyl]-1-piperazinyl}propionamide
N-{2-[(S)-2,6-dioxo-3-piperidyl]-1-oxo-5-isoindolinyl}{4-[4-(4,6-diaza-5-indanylamino)-2-(trifluoromethyl)phenyl]-1-piperazinyl}acetamide
N-{2-[(S)-2,6-dioxo-3-piperidyl]-1-oxo-5-isoindolinyl}{3-[2-chloro-4-(4-methyl-2-pyrimidinylamino)phenyl]-1-azetidinyl}acetamide
N-{2-[(S)-2,6-dioxo-3-piperidyl]-1-oxo-5-isoindolinyl}{3-[2-chloro-4-(4,6-dimethyl-2-pyrimidinylamino)phenyl]-1-azetidinyl}acetamide N-[2-(2,6-dioxo-3-piperidyl)-1,3-dioxo-5-isoindolinyl]{3-[4-(N-methyl-N-2-pyrimidinylamino)-2-(trifluoromethyl)phenyl]-1-azetidinyl}acetamide N-[2-(2,6-dioxo-3-piperidyl)-1,3-dioxo-5-isoindolinyl]{3-[4-(N-methyl-N-2-pyrimidinylamino)-2-chlorophenyl]-1-azetidinyl}acetamide
N-{2-[(S)-2,6-dioxo-3-piperidyl]-1-oxo-5-isoindolinyl}(S)-2-{4-[4-(N-methyl-N-4-pyrimidinylamino)-2-chlorophenyl]-1-piperazinyl}propionamide
N-{2-[(S)-2,6-dioxo-3-piperidyl]-1-oxo-5-isoindolinyl}(S)-2-{4-[4-(4,6-diaza-5-indanylamino)-2-(trifluoromethyl)phenyl]-1-piperazinyl}propionamide
N-{2-[(S)-2,6-dioxo-3-piperidyl]-1-oxo-5-isoindolinyl}(3-{4-[(6-chloro-4-pyrimidinyl)-N-methylamino]-2-(trifluoromethyl)phenyl}-1-azetidinyl)acetamide
N-{2-[(S)-2,6-dioxo-3-piperidyl]-1-oxo-5-isoindolinyl}(R)-2-(3-{4-[(6-chloro-4-pyrimidinyl)-N-methylamino]-2-(trifluoromethyl)phenyl}-1-azetidinyl)propionamide
N-{2-[(S)-2,6-dioxo-3-piperidyl]-1-oxo-5-isoindolinyl}2-{4-[4-(4,6-dimethyl-2-pyrimidinylamino)-2-(trifluoromethyl)phenyl]-1-piperazinyl}-2-methylpropionamide
N-{2-[(S)-2,6-dioxo-3-piperidyl]-1-oxo-5-isoindolinyl}(3-{4-[(4,6-dimethyl-2-pyrimidinyl)-N-methylamino]-2-(trifluoromethyl)phenyl}-1-azetidinyl)acetamide
N-{2-[(S)-2,6-dioxo-3-piperidyl]-1-oxo-5-isoindolinyl}{3-[4-chloro-2-(trifluoromethyl)phenyl]-1-azetidinyl}acetamide
N-{2-[(S)-2,6-dioxo-3-piperidyl]-1-oxo-5-isoindolinyl}2-{3-[2-chloro-4-(4,6-dimethyl-2-pyrimidinylamino)phenyl]-1-azetidinyl}-2-methylpropionamide
N-{2-[(S)-2,6-dioxo-3-piperidyl]-1-oxo-5-isoindolinyl}(4-{2-chloro-4-[(6-chloro-4-pyrimidinyl)-N-methylamino]phenyl}-1-piperazinyl)acetamide
N-{2-[(S)-2,6-dioxo-3-piperidyl]-1-oxo-5-isoindolinyl}(4-{4-[(4,6-dimethyl-2-pyrimidinyl)-N-methylamino]-2-(trifluoromethyl)phenyl}-1-piperazinyl)acetamide
N-{2-[(S)-2,6-dioxo-3-piperidyl]-1-oxo-5-isoindolinyl}(4-{4-[N-methyl(4-methyl-2-pyrimidinyl)amino]-2-(trifluoromethyl)phenyl}-1-piperazinyl)acetamide
N-{2-[(S)-2,6-dioxo-3-piperidyl]-1-oxo-5-isoindolinyl}(S)-2-(4-{4-[(4,6-dimethyl-2-pyrimidinyl)-N-methylamino]-2-(trifluoromethyl)phenyl}-1-piperazinyl)propionamide
N-{2-[(S)-2,6-dioxo-3-piperidyl]-1-oxo-5-isoindolinyl}(S)-2-(4-{4-[N-methyl(4-methyl-2-pyrimidinyl)amino]-2-(trifluoromethyl)phenyl}-1-piperazinyl)propionamide
N-{2-[(S)-2,6-dioxo-3-piperidyl]-1-oxo-5-isoindolinyl}[3-(2,4-dichlorophenyl)-1-azetidinyl]acetamide
N-{2-[(S)-2,6-dioxo-3-piperidyl]-1-oxo-5-isoindolinyl}{3-[2-chloro-4-(trifluoromethyl)phenyl]-1-azetidinyl}acetamide
N-{2-[(S)-2,6-dioxo-3-piperidyl]-1-oxo-5-isoindolinyl}(S)-2-{4-[4-(N-methyl-N-2-pyrimidinylamino)-2-(trifluoromethyl)phenyl]-1-piperazinyl}butyramide
N-{2-[(S)-2,6-dioxo-3-piperidyl]-1-oxo-5-isoindolinyl}(S)-2-(4-{4-[(4,6-dimethyl-2-pyrimidinyl)-N-methylamino]-2-(trifluoromethyl)phenyl}-1-piperazinyl)butyramide
N-{2-[(S)-2,6-dioxo-3-piperidyl]-1-oxo-5-isoindolinyl}{4-[4-(2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}1-{4-[4-(2-pyrimidinylamino)-2-(trifluoromethyl)phenyl]-1-piperazinyl}cyclopropanecarboxamide
2-[3-[4-[(6-chloropyrimidin-4-yl)amino]-2-(trifluoromethyl)phenyl]azetidin-1-yl]-N-[2-[(3S)-2,6-dioxo-3-piperidyl]-1-oxo-isoindolin-5-yl]acetamide
N-{2-[(S)-2,6-dioxo-3-piperidyl]-1-oxo-5-isoindolinyl}{3-[4-(2-methyl-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(4-methyl-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(6-methyl-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(4-chloro-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-methyl-2-pyrimidinylamino)-2-(trifluoromethyl)phenyl]-1-azetidinyl}acetamide
N-{2-[(S)-2,6-dioxo-3-piperidyl]-1-oxo-5-isoindolinyl}1-{4-[4-(4,6-dimethyl-2-pyrimidinylamino)-2-(trifluoromethyl)phenyl]-1-piperazinyl}cyclopropanecarboxamide
N-{2-[(S)-2,6-dioxo-3-piperidyl]-1-oxo-5-isoindolinyl}(R)-2-{3-[4-(2-pyrimidinylamino)-2-(trifluoromethyl)phenyl]-1-azetidinyl}butyramide
N-{2-[(S)-2,6-dioxo-3-piperidyl]-1-oxo-5-isoindolinyl}{3-[4-(4-cyano-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(4,6-dimethyl-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-cyano-2-pyridylamino)-2-(trifluoromethyl)phenyl]-1-azetidinyl}acetamide
N-{2-[(S)-2,6-dioxo-3-piperidyl]-1-oxo-5-isoindolinyl}{3-[4-(5-fluoro-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{4-[2,4-bis(trifluoromethyl)phenyl]-1-piperazinyl}acetamide
N-{2-[(S)-2,6-dioxo-3-piperidyl]-1-oxo-5-isoindolinyl}{3-[4-(6-fluoro-4-pyrimidinylamino)-2-(trifluoromethyl)phenyl]-1-azetidinyl}acetamide
N-{2-[(S)-2,6-dioxo-3-piperidyl]-1-oxo-5-isoindolinyl}{3-[4-(2-furyl)-2-(trifluoromethyl)phenyl]-1-azetidinyl}acetamide
N-{2-[(S)-2,6-dioxo-3-piperidyl]-1-oxo-5-isoindolinyl}{4-[4-(N-ethyl-N-2-pyrimidinylamino)-2-(trifluoromethyl)phenyl]-1-piperazinyl}acetamide
N-{2-[(S)-2,6-dioxo-3-piperidyl]-1-oxo-5-isoindolinyl}{3-[4-(2-pyridyl)-2-(trifluoromethyl)phenyl]-1-azetidinyl}acetamide
N-{2-[(S)-2,6-dioxo-3-piperidyl]-1-oxo-5-isoindolinyl}(3-{4-[N-methyl(4-methyl-2-pyrimidinyl)amino]-2-(trifluoromethyl)phenyl}-1-azetidinyl)acetamide
N-{2-[(S)-2,6-dioxo-3-piperidyl]-1-oxo-5-isoindolinyl}(3-{4-[N-methyl(5-methyl-2-pyrimidinyl)amino]-2-(trifluoromethyl)phenyl}-1-azetidinyl)acetamide
N-{2-[(S)-2,6-dioxo-3-piperidyl]-1-oxo-5-isoindolinyl}{3-[4-(4,6-dimethyl-2-pyrimidinylamino)-2-(trifluoromethyl)phenyl]-1-pyrrolidinyl}acetamide
N-{2-[(S)-2,6-dioxo-3-piperidyl]-1-oxo-5-isoindolinyl}(4-{4-[N-methyl(6-methyl-4-pyrimidinyl)amino]-2-(trifluoromethyl)phenyl}-1-piperazinyl)acetamide
N-{2-[(S)-2,6-dioxo-3-piperidyl]-1-oxo-5-isoindolinyl}(4-{4-[(6-chloro-4-pyrimidinyl)-N-methylamino]-2-(trifluoromethyl)phenyl}-1-piperazinyl)acetamide
N-{2-[(S)-2,6-dioxo-3-piperidyl]-1-oxo-5-isoindolinyl}{3-[4-(2-pyrimidinylamino)-2-(trifluoromethyl)phenyl]-1-pyrrolidinyl}acetamide
N-{2-[(S)-2,6-dioxo-3-piperidyl]-1-oxo-5-isoindolinyl}(3-{4-[N-(²H₃)methyl-N-2-pyrimidinylamino]-2-(trifluoromethyl)phenyl}-1-azetidinyl)acetamide
N-{2-[(S)-2,6-dioxo-3-piperidyl]-1-oxo-5-isoindolinyl}1-{4-[4-(4-methyl-2-pyrimidinylamino)-2-(trifluoromethyl)phenyl]-1-piperazinyl}cyclopropanecarboxamide
N-{2-[(S)-2,6-dioxo-3-piperidyl]-1-oxo-5-isoindolinyl}[3-(4-bromo-2-trifluoromethoxyphenyl)-1-azetidinyl]acetamide
N-{2-[(S)-2,6-dioxo-3-piperidyl]-1-oxo-5-isoindolinyl}{3-[4-(2-pyrimidinylamino)-2-trifluoromethoxyphenyl]-1-azetidinyl}acetamide
N-{2-[(S)-2,6-dioxo-3-piperidyl]-1-oxo-5-isoindolinyl}{3-[4-(N-methyl-N-2-pyrimidinylamino)-2-trifluoromethoxyphenyl]-1-azetidinyl}acetamide
N-{2-[(S)-2,6-dioxo-3-piperidyl]-1-oxo-5-isoindolinyl}{4-[5-bromo-3-(trifluoromethyl)-2-pyridyl]-1-piperazinyl}acetamide
N-{2-[(S)-2,6-dioxo-3-piperidyl]-1-oxo-5-isoindolinyl}{4-[5-(N-methyl-N-2-pyrimidinylamino)-3-(trifluoromethyl)-2-pyridyl]-1-piperazinyl}acetamide
N-{2-[(S)-2,6-dioxo-3-piperidyl]-1-oxo-5-isoindolinyl}(4-{5-[(4-chloro-6-methyl-2-pyrimidinyl)-N-methylamino]-3-(trifluoromethyl)-2-pyridyl}-1-piperazinyl)acetamide
N-{2-[(S)-2,6-dioxo-3-piperidyl]-1-oxo-5-isoindolinyl}{4-[4-(2-pyrimidinylamino)-2-(trifluoromethyl)phenyl]-1,2,3,6-tetrahydro-1-pyridyl}acetamide
N-{2-[(S)-2,6-dioxo-3-piperidyl]-1-oxo-5-isoindolinyl}[3-(2-chloro-4-trifluoromethoxyphenyl)-1-azetidinyl]acetamide
N-{2-[(S)-2,6-dioxo-3-piperidyl]-1-oxo-5-isoindolinyl}{3-[4-fluoro-2-(trifluoromethyl)phenyl]-1-azetidinyl}acetamide
N-{2-[(S)-2,6-dioxo-3-piperidyl]-1-oxo-5-isoindolinyl}[3-(4-chloro-2-trifluoromethoxyphenyl)-1-azetidinyl]acetamide
N-{2-[(S)-2,6-dioxo-3-piperidyl]-1-oxo-5-isoindolinyl}{4-[4-(4-methyl-2-pyrimidinylamino)-2-(trifluoromethyl)phenyl]-1-piperidyl}acetamide
N-{2-[(S)-2,6-dioxo-3-piperidyl]-1-oxo-5-isoindolinyl}{6-[4-(2-pyrimidinylamino)-2-(trifluoromethyl)phenyl]-2,6-diaza-2-spiro[3.3]heptyl}acetamide
N-{2-[(S)-2,6-dioxo-3-piperidyl]-1-oxo-5-isoindolinyl}{4-[4-(2-pyrimidinylamino)-2-(trifluoromethyl)phenyl]-1-piperidyl}acetamide
N-{2-[(S)-2,6-dioxo-3-piperidyl]-1-oxo-5-isoindolinyl}{4-[3,5-bis(trifluoromethyl)-2-pyridyl]-1-piperazinyl}acetamide
N-{2-[(S)-2,6-dioxo-3-piperidyl]-1-oxo-5-isoindolinyl}1-(4-{4-[(4,6-dimethyl-2-pyrimidinyl)-N-(²H₃)methylamino]-2-(trifluoromethyl)phenyl}-1-piperazinyl)cyclopropanecarboxamide
N-{2-[(S)-2,6-dioxo-3-piperidyl]-1-oxo-5-isoindolinyl}[4-(2-chloro-4-trifluoromethoxyphenyl)-1-piperazinyl]acetamide
N-{2-[(S)-2,6-dioxo-3-piperidyl]-1-oxo-5-isoindolinyl}{3-[4-bromo-2-(trifluoromethyl)phenyl]-1-azetidinyl}acetamide
N-[2-(2,6-dioxo-3-piperidyl)-6-fluoro-1-oxo-5-isoindolinyl]{4-[4-(N-methyl-N-2-pyrimidinylamino)-2-(trifluoromethyl)phenyl]-1-piperazinyl}acetamide
N-[2-(2,6-dioxo-3-piperidyl)-6-fluoro-1-oxo-5-isoindolinyl]{4-[2,4-bis(trifluoromethyl)phenyl]-1-piperazinyl}acetamide
N-[2-(2,6-dioxo-3-piperidyl)-6-fluoro-1-oxo-5-isoindolinyl]{3-[2-chloro-4-(trifluoromethyl)phenyl]-1-azetidinyl}acetamide
N-{2-[(S)-2,6-dioxo-3-piperidyl]-1-oxo-5-isoindolinyl}(4-{5-[N-(²H₃)methyl-N-2-pyrimidinylamino]-3-(trifluoromethyl)-2-pyridyl}-1-piperazinyl)acetamide
N-{2-[(S)-2,6-dioxo-3-piperidyl]-1-oxo-5-isoindolinyl}{4-[4-(2-pyridyloxy)-2-(trifluoromethyl)phenyl]-1-piperazinyl}acetamide
N-{2-[(S)-2,6-dioxo-3-piperidyl]-1-oxo-5-isoindolinyl}{4-[4-(4,6-dimethyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{3-methyl-3-[4-(2-pyrimidinylamino)-2-(trifluoromethyl)phenyl]-1-azetidinyl}acetamide
N-{2-[(S)-2,6-dioxo-3-piperidyl]-1-oxo-5-isoindolinyl}{4-[2-chloro-4-(4,6-dimethyl-2-pyrimidinyloxy)phenyl]-1-piperazinyl}acetamide
N-{2-[(S)-2,6-dioxo-3-piperidyl]-1-oxo-5-isoindolinyl}{4-[4-(4-methyl-2-pyrimidinyloxy)-2-(trifluoromethyl)phenyl]-1-piperazinyl}acetamide
N-{2-[(S)-2,6-dioxo-3-piperidyl]-1-oxo-5-isoindolinyl}{4-[2-chloro-4-(4-methyl-2-pyrimidinyloxy)phenyl]-1-piperazinyl}acetamide
N-{2-[(S)-2,6-dioxo-3-piperidyl]-1-oxo-5-isoindolinyl}{4-[2-chloro-4-(6-methyl-4-pyrimidinyloxy)phenyl]-1-piperazinyl}acetamide
N-{2-[(S)-2,6-dioxo-3-piperidyl]-1-oxo-5-isoindolinyl}{4-[4-(6-methyl-4-pyrimidinyloxy)-2-(trifluoromethyl)phenyl]-1-piperazinyl}acetamide
N-{2-[(S)-2,6-dioxo-3-piperidyl]-6-fluoro-1-oxo-5-isoindolinyl}{4-[2,4-bis(trifluoromethyl)phenyl]-1-piperazinyl}acetamide
N-{2-[(S)-2,6-dioxo-3-piperidyl]-6-fluoro-1-oxo-5-isoindolinyl}{4-[2-chloro-4-(2-pyridylamino)phenyl]-1-piperazinyl}acetamide
N-{2-[(S)-2,6-dioxo-3-piperidyl]-6-fluoro-1-oxo-5-isoindolinyl}{4-[2-chloro-4-(4-methyl-2-pyrimidinyloxy)phenyl]-1-piperazinyl}acetamide
N-{2-[(S)-2,6-dioxo-3-piperidyl]-6-fluoro-1-oxo-5-isoindolinyl}{4-[2-chloro-4-(6-methyl-4-pyrimidinyloxy)phenyl]-1-piperazinyl}acetamide
N-{2-[(S)-2,6-dioxo-3-piperidyl]-6-fluoro-1-oxo-5-isoindolinyl}{4-[4-(4-methyl-2-pyrimidinyloxy)-2-(trifluoromethyl)phenyl]-1-piperazinyl}acetamide
N-[2-(2,6-dioxo-3-piperidyl)-4-fluoro-1-oxo-5-isoindolinyl]{4-[2-chloro-4-(2-pyridylamino)phenyl]-1-piperazinyl}acetamide
N-[2-(2,6-dioxo-3-piperidyl)-4-fluoro-1-oxo-5-isoindolinyl]{4-[2,4-bis(trifluoromethyl)phenyl]-1-piperazinyl}acetamide
N-[2-(2,6-dioxo-3-piperidyl)-4-fluoro-1-oxo-5-isoindolinyl]{4-[2-chloro-4-(6-methyl-4-pyrimidinyloxy)phenyl]-1-piperazinyl}acetamide
N-[2-(2,6-dioxo-3-piperidyl)-4-fluoro-1-oxo-5-isoindolinyl]{4-[2-chloro-4-(4-methyl-2-pyrimidinyloxy)phenyl]-1-piperazinyl}acetamide
N-[2-(2,6-dioxo-3-piperidyl)-4-fluoro-1-oxo-5-isoindolinyl]{4-[4-(4-methyl-2-pyrimidinyloxy)-2-(trifluoromethyl)phenyl]-1-piperazinyl}acetamide
N-{2-[(S)-2,6-dioxo-3-piperidyl]-6-fluoro-1-oxo-5-isoindolinyl}{3-[4-(2-pyrimidinyloxy)-2-(trifluoromethyl)phenyl]-1-azetidinyl}acetamide
N-{2-[(S)-2,6-dioxo-3-piperidyl]-6-fluoro-1-oxo-5-isoindolinyl}{3-[4-(6-methyl-4-pyrimidinyloxy)-2-(trifluoromethyl)phenyl]-1-azetidinyl}acetamide
N-{2-[(S)-2,6-dioxo-3-piperidyl]-6-fluoro-1-oxo-5-isoindolinyl}{3-[2-chloro-4-(6-methyl-4-pyrimidinyloxy)phenyl]-1-azetidinyl}acetamide
N-{2-[(S)-2,6-dioxo-3-piperidyl]-6-fluoro-1-oxo-5-isoindolinyl}{3-[2-chloro-4-(2-pyrimidinyloxy)phenyl]-1-azetidinyl}acetamide
N-{2-[(S)-2,6-dioxo-3-piperidyl]-1-oxo-5-isoindolinyl}{3-[2-chloro-4-(methylamino)phenyl]-1-azetidinyl}acetamide
N-{2-[(S)-2,6-dioxo-3-piperidyl]-1-oxo-5-isoindolinyl}{3-[4-(methylamino)-2-(trifluoromethyl)phenyl]-1-azetidinyl}acetamide
N-{2-[(R)-2,6-dioxo-3-piperidyl]-4-fluoro-1-oxo-5-isoindolinyl}{3-[2-chloro-4-(6-methyl-4-pyrimidinyloxy)phenyl]-1-azetidinyl}acetamide
N-{2-[(S)-2,6-dioxo-3-piperidyl]-4-fluoro-1-oxo-5-isoindolinyl}{3-[2-chloro-4-(2-pyrimidinyloxy)phenyl]-1-azetidinyl}acetamide
N-[2-(2,6-dioxo-3-piperidyl)-3-oxo-2,5-diaza-6-indanyl]{4-[2-chloro-4-(2-pyridylamino)phenyl]-1-piperazinyl}acetamide
N-[2-(2,6-dioxo-3-piperidyl)-1-oxo-2,4-diaza-5-indanyl]{4-[2-chloro-4-(2-pyridylamino)phenyl]-1-piperazinyl}acetamide
3-{5-[2-(4-{4-[5-(benzyloxy)-2-pyridylamino]-2-(trifluoromethyl)phenyl}-1-piperazinyl)ethoxy]-1-oxo-2-isoindolinyl}-2,6-piperidinedione
3-[5-(2-{4-[2,4-bis(trifluoromethyl)phenyl]-1-piperidyl}ethoxy)-1-oxo-2-isoindolinyl]-2,6-piperidinedione
3-[5-(2-{3-[2,4-bis(trifluoromethyl)phenyl]-1-azetidinyl}ethoxy)-1-oxo-2-isoindolinyl]-2,6-piperidinedione,
or a pharmaceutically acceptable salt, diastereomer, enantiomer, racemate, hydrate, solvate, or prodrug thereof.

15. A pharmaceutical composition comprising:
(a) a first component selected from the group consisting of the compound, salt, diastereomer, enantiomer, racemate, hydrate, solvate, prodrug of claim 1, or a combination thereof; and
(b) a second component selected from the group consisting of a pharmaceutically acceptable carrier, diluent, excipient, adjuvant, or a combination thereof.

16. A method of treating cancer of a subject, which comprises administering to the subject a therapeutically effective amount of the composition of claim 15, wherein the composition degrades a target protein via cereblon, thereby decreasing GSPT1, MYC, or both.

17. The method of claim 16, wherein the cancer is selected from the group consisting of: adenocarcinoma, adrenocortical cancer, bladder cancer, bone cancer, brain cancer, breast cancer, cancer of the buccal cavity, cervical cancer, colon cancer, colorectal cancer, endometrial or uterine carcinoma, epidermoid carcinoma, esophageal cancer, eye cancer, follicular carcinoma, gallbladder cancer, gastrointestinal cancer, cancer of the genitourinary tract, glioblastoma, hairy cell carcinoma, head and neck cancer, hepatic carcinoma, hepatocellular cancer, Hodgkin's disease, keratoacanthoma, kidney cancer, large cell carcinoma, cancer of the large intestine, laryngeal cancer, liver cancer, adenocarcinoma of the lung, small-cell lung cancer, squamous carcinoma of the lung, non-small cell lung cancer, melanoma, a myeloproliferative disorder, neuroblastoma, ovarian cancer, papillary carcinoma, pancreatic cancer, cancer of the peritoneum, prostate cancer, rectal cancer, salivary gland carcinoma, sarcoma, squamous cell cancer, small cell carcinoma, cancer of the small intestine, stomach cancer, testicular cancer, thyroid cancer, and vulval cancer.
